(19) United States
(12) Patent Application Publication
(10) Pub. No.: US 2024/0360114 A1
(43) Pub. Date: Oct. 31, 2024

(54) FERROPTOSIS MODULATORS, PREPARATIONS, AND USES THEREOF

(71) Applicant: SIRONAX LTD., Grand Cayman (KY)

(72) Inventors: Jianguang HAN, Beijing (CN); Weijie HOU, Beijing (CN); Zhiyuan ZHANG, Beijing (CN); Yanping XU, Beijing (CN); Yimin JIANG, Beijing (CN); Xingyu XU, Beijing (CN)

(21) Appl. No.: 18/293,357

(22) PCT Filed: Aug. 9, 2022
(86) PCT No.: PCT/CN2022/111129

§ 371 Date: Jan. 30, 2024

(30) Foreign Application Priority Data
Aug. 9, 2021 (WO) PCT/CN2021/111481

(51) Int. Cl. C07D 413/04; A61K 31/4427; A61K 31/47; A61K 31/496; A61K 31/517; A61K 31/5377; A61K 31/553; C07C 211/48; C07D 213/74; C07D 215/10; C07D 215/16; C07D 241/04; C07D 265/30; C07D 401/04; C07D 413/14; C07D 487/08; C07D 491/048; C07D 491/107

(52) U.S. Cl. CPC C07D 413/04; A61K 31/4427; A61K 31/47; A61K 31/496; A61K 31/517; A61K 31/5377; A61K 31/553; C07C 211/48; C07D 213/74; C07D 215/10; C07D 215/16; C07D 241/04; C07D 265/30; C07D 401/04; C07D 413/14; C07D 487/08; C07D 491/048; C07D 491/107

(57) ABSTRACT

This disclosure provides compounds of Formula I, compositions comprising the same, and methods of using the same, including use in treating various diseases and conditions, e.g., those involving ferroptosis.

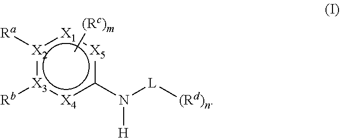

### FIELD OF THE DISCLOSURE

The present disclosure relates to compounds that modulate ferroptosis, compositions comprising the compounds, methods of preparing the compounds, and methods of using the compounds to treat various diseases or conditions, e g, those involving ferroptosis.

### BACKGROUND OF THE DISCLOSURE

Ferroptosis is a type of programmed cell death dependent on iron and characterized by the accumulation of lipid peroxides, and is genetically and biochemically distinct from other forms of regulated cell death such as apoptosis, autophagy, and necrosis. (Dixon et al., Cell 149, 1060-1072 (2012).) Certain mechanisms of ferroptosis and key contributors to ferroptotic cell death is reviewed and illustrated by Conrad et al., Nature Chemical Biology, vol. 15, December 2019, 1137-1147.

Ferroptosis has been implicated in a number of diseases or conditions, including neuropathy, stroke, neurodegenerative disease (e.g., Alzheimer's disease), Parkinson's Disease, amyotrophic lateral sclerosis (AML), multiple sclerosis, Huntington's Disease, dementia with Lewy bodies, Friedreich's ataxia, hair follicle morphogenesis, diabetes, sepsis, transplant rejection, periventricular leukomalacia, ischemia reperfusion injury, blood coagulation, myocardial infarction, and kidney dysfunction such as acute kidney failure. For example, inactivation of the the ferroptosis regulator Gpx4 triggers acute renal failure in mice. (Angeli, et al., Nature Cell Biology, published online Nov. 17, 2014; DOI: 10.1038/ncb3064.) Ferroptosis has been considered to be implicated in several pathophysiological contexts such as tumor suppression, antiviral immunity, neurodegeneration, and ischemia/reperfusion injury (IRI). (Angeli et al., Trends in Pharmacological Sciences, May 2017, Vol. 38, No. 5, 489-498 (2017).)

Certain compounds that modulate ferroptosis are disclosed by Skouta et al., J. Am. Chem. Soc., dx.doi.org/10.1021/ja411006a; and in WO2013152039A1. WO2015084749A1, WO2019154795A1, WO2016075137A1, WO2020185738A1, and PCT/CN2021/078601.

### SUMMARY OF THE DISCLOSURE

One aspect of this disclosure provides a compound selected from compounds of the Formulae disclosed herein, a tautomer thereof, a solvate or stereoisomer of the compound or the tautomer, or a pharmaceutically acceptable salt of the foregoing, which can be employed in the treatment of various diseases or conditions, such as diseases or conditions involving ferroptosis. For example, disclosed herein is a compound of the following structural Formula I:

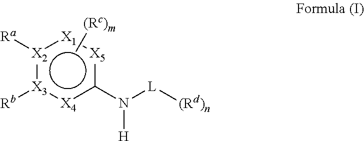

a tautomer thereof, a solvate or stereoisomer of the compound or the tautomer, or a pharmaceutically acceptable salt of the foregoing, wherein:
L is cycloalkyl, heterocyclyl, or C;
X₁, X₂, X₃, X₄, and X₅ are each independently C or N;
R^{a} is absent or selected from H, halogen, CN, optionally substituted heteroatom, optionally substituted alkyl, optionally substituted alkenyl, optionally substituted alkynyl, optionally substituted acyl, optionally substituted cycloalkyl, optionally substituted heterocyclyl, optionally substituted aryl, and optionally substituted heteroaryl;
R^{b} is absent or selected from H, halogen, CN, optionally substituted heteroatom, optionally substituted alkyl, optionally substituted cycloalkyl, and optionally substituted heterocyclyl;
R^{c} and R^{b} may join together to form an optionally substituted 3- to 10-membered carbocyclic, heterocyclic, aromatic, or heteroaromatic ring;
R^{c}, for each occurrence, is independently selected from halogen, optionally substituted heteroatom, and optionally substituted alkyl;
R^{b} and one of R^{c} may join together to form an optionally substituted 3- to 10-membered carbocyclic, heterocyclic, aromatic, or heteroaromatic ring;
R^{d}, for each occurrence, is independently selected from halogen, CN, optionally substituted heteroatom, optionally substituted alkyl, optionally substituted alkenyl, optionally substituted alkynyl, optionally substituted acyl, optionally substituted cycloalkyl, optionally substituted heterocyclyl, optionally substituted aryl, and optionally substituted heteroaryl; and
m and n are each an integer independently selected from 0, 1, 2, and 3.

In one aspect of the disclosure, the compounds of the Formulae disclosed herein are selected from Compounds 1 to 573 shown below, a tautomer thereof, a solvate or stereoisomer of the compound or the tautomer, or a pharmaceutically acceptable salt of the foregoing.

In some embodiments, the disclosure provides pharmaceutical compositions comprising a compound of the Formulae disclosed herein, a tautomer thereof, a solvate or stereoisomer of the compound or the tautomer, or a pharmaceutically acceptable salt of the foregoing, and a pharmaceutically acceptable carrier. In some embodiments, the pharmaceutical compositions may comprise a compound selected from Compounds 1 to 573 shown below, a tautomer thereof, a solvate or stereoisomer of the compound or the tautomer, or a pharmaceutically acceptable salt of the foregoing, and a pharmaceutically acceptable carrier. These compositions may further comprise an additional active pharmaceutical agent.

In one aspect of the disclosure, the compounds of the Formulae disclosed herein are selected from Compounds 574 to 661 shown below, a tautomer thereof, a solvate or stereoisomer of the compound or the tautomer, or a pharmaceutically acceptable salt of the foregoing

In some embodiments, the disclosure provides pharmaceutical compositions comprising a compound of the Formulae disclosed herein, a tautomer thereof, a solvate or stereoisomer of the compound or the tautomer, or a pharmaceutically acceptable salt of the foregoing, and a pharmaceutically acceptable carrier. In some embodiments, the pharmaceutical compositions may comprise a compound selected from Compounds 574 to 661 shown below, a tautomer thereof, a solvate or stereoisomer of the compound or the tautomer, or a pharmaceutically acceptable salt of the foregoing, and a pharmaceutically acceptable carrier. These compositions may further comprise an additional active pharmaceutical agent.

Another aspect of the disclosure provides methods of treating a disease or condition, comprising administering to a subject in need thereof, a therapeutically effective amount of a compound of the Formulae disclosed herein, a tautomer thereof, a solvate or stereoisomer of the compound or the tautomer, or a pharmaceutically acceptable salt of the foregoing, or a pharmaceutical composition comprising any of the foregoing, wherein the disease or condition is selected from neuropathy, stroke, neurodegenerative disease (e.g., Alzheimer's disease), Parkinson's Disease, amyotrophic lateral sclerosis (AML), multiple sclerosis, Huntington's Disease, dementia with Lewy bodies, Friedreich's ataxia, hair follicle morphogenesis, diabetes, sepsis, transplant rejection, periventricular leukomalacia, ischemia reperfusion injury, blood coagulation, myocardial infarction, and kidney dysfunction such as acute kidney failure.

A further aspect of the disclosure provides methods of treating a disease or condition involving ferroptosis, comprising administering to a subject in need thereof, a therapeutically effective amount of a compound of the Formulae disclosed herein, a tautomer thereof, a solvate or stereoisomer of the compound or the tautomer, or a pharmaceutically acceptable salt of the foregoing, or a pharmaceutical composition comprising any of the foregoing.

In some embodiments, the methods of treatment comprise administering to a subject in need thereof, a compound selected from Compounds 1 to 573 shown below, a tautomer thereof, a solvate or stereoisomer of the compound or the tautomer, or a pharmaceutically acceptable salt of the foregoing, or a pharmaceutical composition comprising any of the foregoing.

In some embodiments, the methods of treatment comprise administration of an additional active pharmaceutical agent to the subject in need thereof, either in the same pharmaceutical composition as a compound of the Formulae disclosed herein, a tautomer thereof, a solvate or stereoisomer of the compound or the tautomer, or a pharmaceutically acceptable salt of the foregoing, or in a separate composition. In some embodiments, the methods of treatment comprise administering a compound selected from Compounds 1 to 573 shown below, a tautomer thereof, a solvate or stereoisomer of the compound or the tautomer, or a pharmaceutically acceptable salt of the foregoing with an additional active pharmaceutical agent either in the same pharmaceutical composition or in a separate composition. When administered as a separate composition, the additional therapeutic agent may be administered prior to, at the same time as, or following administration of the compound, tautomer, solvate, stereoisomer, or a pharmaceutically acceptable salt disclosed herein.

Also disclosed herein are methods of modulating, e.g., inhibiting, ferroptosis in a subject in need thereof, comprising contacting the subject with a compound of the Formulae disclosed herein, a tautomer thereof, a solvate or stereoisomer of the compound or the tautomer, or a pharmaceutically acceptable salt of the foregoing, or a pharmaceutical composition comprising any of the foregoing. In some embodiments, the methods of modulating, e.g., inhibiting, ferroptosis in a subject in need thereof comprise contacting the subject with a compound selected from Compounds 1 to 573 shown below, a tautomer thereof, a solvate or stereoisomer of the compound or the tautomer, or a pharmaceutically acceptable salt of the foregoing, or a pharmaceutical composition comprising any of the foregoing.

In some embodiments, the methods of treatment comprise administering to a subject in need thereof, a compound selected from Compounds 574 to 661 shown below, a tautomer thereof, a solvate or stereoisomer of the compound or the tautomer, or a pharmaceutically acceptable salt of the foregoing, or a pharmaceutical composition comprising any of the foregoing.

In some embodiments, the methods of treatment comprise administration of an additional active pharmaceutical agent to the subject in need thereof, either in the same pharmaceutical composition as a compound of the Formulae disclosed herein, a tautomer thereof, a solvate or stereoisomer of the compound or the tautomer, or a pharmaceutically acceptable salt of the foregoing, or in a separate composition. In some embodiments, the methods of treatment comprise administering a compound selected from Compounds 574 to 661 shown below, a tautomer thereof, a solvate or stereoisomer of the compound or the tautomer, or a pharmaceutically acceptable salt of the foregoing with an additional active pharmaceutical agent either in the same pharmaceutical composition or in a separate composition. When administered as a separate composition, the additional therapeutic agent may be administered prior to, at the same time as, or following administration of the compound, tautomer, solvate, stereoisomer, or a pharmaceutically acceptable salt disclosed herein. Also disclosed herein are methods of modulating, e.g., inhibiting, ferroptosis in a subject in need thereof, comprising contacting the subject with a compound of the Formulae disclosed herein, a tautomer thereof, a solvate or stereoisomer of the compound or the tautomer, or a pharmaceutically acceptable salt of the foregoing, or a pharmaceutical composition comprising any of the foregoing. In some embodiments, the methods of modulating, e.g., inhibiting, ferroptosis in a subject in need thereof comprise contacting the subject with a compound selected from Compounds 574 to 661 shown below, a tautomer thereof, a solvate or stereoisomer of the compound or the tautomer, or a pharmaceutically acceptable salt of the foregoing, or a pharmaceutical composition comprising any of the foregoing.

### DETAILED DESCRIPTION OF THE DISCLOSURE

### I. Definitions

The term “a” or “an” when referring to a noun as used herein encompasses the expression “at least one” and therefore encompasses both singular and plural units of the noun. For example, “an additional pharmaceutical agent” means a single or two or more additional pharmaceutical agents.

The term “alkyl” refers to a hydrocarbon group selected from linear and branched saturated hydrocarbon groups, containing 1-20, e.g., 1-18, 1-12, 1-10, 1-8, 1-6, 1-4, or 1-3, carbon atoms. Examples of the alkyl group include methyl, ethyl, 1-propyl or n-propyl (“n-Pr”), 2-propyl or isopropyl (“i-Pr”), 1-butyl or n-butyl (“n-Bu”), 2-methyl-1-propyl or isobutyl (“i-Bu”), 1-methylpropyl or s-butyl (“s-Bu”), and 1,1-dimethylethyl or t-butyl (“t-Bu”). Other examples of an alkyl group include 1-pentyl, 2-pentyl. 3-pentyl, 2-methyl-2-butyl, 3-methyl-2-butyl, 3-methyl-1-butyl, 2-methyl-1-butyl, 1-hexyl, 2-hexyl, 3-hexyl, 2-methyl-2-pentyl, 3-methyl-2-pentyl, 4-methyl-2-pentyl, 3-methyl-3-pentyl, 2-methyl-3-pentyl, 2,3-dimethyl-2-butyl, and 3,3-dimethyl-2-butyl groups. Lower alkyl contains 1-8, preferably 1-6, more preferably 1-4 carbon atoms, and more preferably 1-3 carbon atoms.

The term “alkenyl” refers to a hydrocarbon group selected from linear and branched hydrocarbon groups, comprising at least one C═C double bond and 2-20, e.g., 2-18, 2-12, 2-10, 2-8, 2-6, or 2-4, carbon atoms. Examples of the alkenyl group may be selected from ethenyl or vinyl, prop-1-enyl, prop-2-enyl, 2-methylprop-1-enyl, but-1-enyl, but-2-enyl, but-3-enyl, buta-1,3-dienyl, 2-methylbuta-1,3-diene, hex-1-enyl, hex-2-enyl, hex-3-enyl, hex-4-enyl, and hexa-1,3-dienyl groups. Lower alkenyl contains 2-8, preferably 2-6, and more preferably 2-4 carbon atoms.

The term “alkynyl” refers to a hydrocarbon group selected from linear and branched hydrocarbon groups, comprising at least one C≡C triple bond and 2-20, e.g., 2-18, 2-12, 2-10, 2-8, 2-6, or 2-4, carbon atoms. Examples of the alkynyl group include ethynyl, 1-propynyl, 2-propynyl (propargyl), 1-butynyl, 2-butynyl, and 3-butynyl groups. Lower alkynyl contains 2-8, preferably 2-6, and more preferably 2-4 carbon atoms.

The term “heteroalkyl” refers to an alkyl group, as defined herein, in which one or more of the constituent carbon atoms have been replaced by a heteroatom, e.g., nitrogen, oxygen, or sulfur, e.g., CH₃CH₂OH, CH₃CH₂OC₂H₅, CH₃CH₂SH, CH₃CH₂SC₂H₅, CH₃CH₂NH₂, CH₃CH₂NHC₂H₅, etc. In some embodiments, in addition to the replacement of one or more of the constituent carbon atoms by nitrogen, oxygen, or sulfur, a heteroalkyl group is further optionally substituted as defined herein.

The term “cycloalkyl” refers to a hydrocarbon group selected from saturated and partially unsaturated cyclic hydrocarbon groups, e.g., monocyclic and polycyclic (e.g., bicyclic and tricyclic) groups. For example, the cycloalkyl group may be of 3-12, 3 to 10, or 3-8, or 3-6, or 3-4, or 5-6 carbon atoms. Even further for example, the cycloalkyl group may be a monocyclic group of 3-12, or 3-8, or 3-6, or 3-4, or 5-6 carbon atoms. Examples of the monocyclic cycloalkyl group include cyclopropyl, cyclobutyl, cyclopentyl, 1-cyclopent-1-enyl, 1-cyclopent-2-enyl, 1-cyclopent-3-enyl, cyclohexyl, 1-cyclohex-1-enyl, 1-cyclohex-2-enyl, 1-cyclohex-3-enyl, cyclohexadienyl, cycloheptyl, cyclooctyl, cyclononyl, cyclodecyl, cycloundecyl, and cyclododecyl groups. Examples of the bicyclic cycloalkyl groups include those having 7-12 ring atoms arranged as a bicycle ring selected from [4,4], [4,5], [5,5], [5,6] and [6,6] ring systems, or as a bridged bicyclic ring selected from bicyclo[2.2.1]heptane, bicyclo[2.2.2]octane, and bicyclo[3.2.2]nonane. The ring may be saturated or have at least one double bond (i.e., partially unsaturated), but is not fully conjugated, and is not aromatic, as aromatic is defined herein.

The term “heterocyclic” or “heterocycle” or “heterocyclyl” refers to a ring selected from 4- to 12-membered, e.g., 3- to 6-membered, 3- to 5-membered, 4- to 5-membered, or 5- to 6-membered, monocyclic, bicyclic, and tricyclic, saturated and partially unsaturated rings comprising at least one carbon atom in addition to 1, 2, 3, or 4 heteroatoms, selected from, e.g., oxygen, sulfur, nitrogen, and silicon. “Heterocycle” also refers to a 5- to 7-membered heterocyclic ring comprising at least one heteroatom selected from N, O, and S fused with 5-, 6-, and/or 7-membered cycloalkyl, carbocyclic aromatic, or heteroaromatic ring, provided that the point of attachment is at the heterocyclic ring when the heterocyclic ring is fused with a carbocyclic aromatic or a heteroaromatic ring, and that the point of attachment can be at the cycloalkyl or heterocyclic ring when the heterocyclic ring is fused with cycloalkyl. “Heterocycle” also refers to an aliphatic spirocyclic ring comprising at least one heteroatom selected from N, O, and S, provided that the point of attachment is at the heterocyclic ring. The rings may be saturated or have at least one double bond (i.e., partially unsaturated). A heterocycle may be substituted with oxo. The point of the attachment may be carbon or heteroatom in the heterocyclic ring. A heterocycle is not a heteroaryl as defined herein. Examples of heterocycles include, but are not limited to, (as numbered from the linkage position assigned priority 1) 1-pyrrolidinyl, 2-pyrrolidinyl, 2,4-imidazolidinyl, 2,3-pyrazolidinyl, 1-pipcridinyl, 2-piperidinyl, 3-piperidinyl, 4-pipcridinyl, 2,5-pipcrazinyl, pyranyl, 2-morpholinyl, 3-morpholinyl, oxiranyl, aziridinyl, thiiranyl, azetidinyl, oxetanyl, thietanyl, 1,2-dithietanyl, 1,3-dithietanyl, dihydropyridinyl, tetrahydropyridinyl, thiomorpholinyl, thioxanyl, piperazinyl, homopiperazinyl, homopiperidinyl, azepanyl, oxepanyl, thiepanyl, 1,4-oxathianyl, 1,4-dioxepanyl, 1,4-oxathiepanyl, 1,4-oxaazepanyl, 1,4-dithiepanyl, 1,4-thiazepanyl, 1,4-diazepanyl, 1,4-dithianyl, 1,4-azathianyl, oxazepinyl, diazepinyl, thiazepinyl, dihydrothienyl, dihydropyranyl, dihydrofuranyl, tetrahydrofuranyl, tetrahydrothienyl, tetrahydropyranyl, tetrahydrothiopyranyl, 1-pyrrolinyl, 2-pyrrolinyl, 3-pyrrolinyl, indolinyl, 2H-pyranyl, 4H-pyranyl, 1,4-dioxanyl, 1,3-dioxolanyl, pyrazolinyl, pyrazolidinyl, dithianyl, dithiolanyl, pyrazolidinylimidazolinyl, pyrimidinonyl, 1, 1-dioxo-thiomorpholinyl, 3-azabicyco [3.1.0]hexanyl, 3-azabicyclo[4.1.0]heptanyl and azabicyclo[2.2.2]hexanyl. Substituted heterocycle also includes ring systems substituted with one or more oxo moieties, such as piperidinyl N-oxide, morpholinyl-N-oxide, 1-oxo-1-thiomorpholinyl, and 1, 1-dioxo-1-thiomorpholinyl.

The term “fused ring” herein refers to a polycyclic ring system, e.g., a bicyclic or tricyclic ring system, in which two rings share only two ring atoms and one bond in common. Examples of fused rings may comprise a fused bicyclic cycloalkyl ring such as those having from 7 to 12 ring atoms arranged as a bicyclic ring selected from [4,4], [4,5], [5,5], [5,6] and [6,6] ring systems as mentioned above; a fused bicyclic aryl ring such as 7 to 12 membered bicyclic aryl ring systems as mentioned above, a fused tricyclic aryl ring such as 10 to 15 membered tricyclic aryl ring systems mentioned above; a fused bicyclic heteroaryl ring such as 8- to 12-membered bicyclic heteroaryl rings as mentioned above, a fused tricyclic heteroaryl ring such as 11- to 14-membered tricyclic heteroaryl rings as mentioned above; and a fused bicyclic or tricyclic heterocyclyl ring as mentioned above.

The term “heteroatom” means one or more of oxygen, sulfur, nitrogen, phosphorus, and silicon, including, any oxidized form of nitrogen or sulfur; the quaternized form of any basic nitrogen or a substitutable nitrogen of a heterocyclic ring, for example N (as in 3,4-dihydro-2H-pyrrolyl), NH (as in pyrrolidinyl) or NR″ (wherein R is, e.g., an optionally substituted alkyl group) (as in N-substituted pyrrolidinyl).

The term “unsaturated”, as used herein, means that a moiety has one or more units or degrees of unsaturation. Unsaturation is the state in which not all of the available valence bonds in a compound are satisfied by substituents and thus the compound contains one or more double or triple bonds.

The term “alkoxy” as used herein, refers to an alkyl group, as defined above, wherein one carbon of the alkyl group is replaced by an oxygen atom, provided that the oxygen atom is linked between two carbon atoms.

The term “halogen” includes F, Cl, Br, and I, i.e., fluoro, chloro, bromo, and iodo, respectively.

As used herein, a “CN,” “cyano” or “nitrile” group refers to —C≡N.

As used herein, an “aromatic ring” refers to a carbocyclic or heterocyclic ring that contains conjugated, planar ring systems with delocalized pi electron orbitals comprised of [4n-2] p orbital electrons, wherein n is an integer of 0 to 6. A “non-aromatic” ring refers to a carbocyclic or heterocyclic that does not meet the requirements set forth above for an aromatic ring, and can be either completely or partially saturated. Non-limiting examples of aromatic rings include aryl and heteroaryl rings that are further defined as follows. An “aromatic ring” may be depicted as a cycle with conjugated double bonds, such as

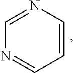

or as a cycle with an inside circle such as

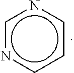

The term “aryl” herein refers to a group selected from: monocyclic carbocyclic aromatic rings, for example, phenyl; bicyclic ring systems such as 7-12 membered bicyclic ring systems wherein at least one ring is carbocyclic and aromatic, selected, for example, from naphthalene, indane, and 1,2,3,4-tetrahydroquinoline; and tricyclic ring systems such as 10-15 membered tricyclic ring systems wherein at least one ring is carbocyclic and aromatic, for example, fluorene.

For example, the aryl group may be a 6-membered carbocyclic aromatic ring fused to a 5- to 7-membered cycloalkyl or heterocyclic ring optionally comprising at least one heteroatom selected from N, O, and S, provided that the point of attachment is at the carbocyclic aromatic ring when the carbocyclic aromatic ring is fused with a heterocyclic ring, and the point of attachment can be at the carbocyclic aromatic ring or at the cycloalkyl group when the carbocyclic aromatic ring is fused with a cycloalkyl group. Bivalent radicals formed from substituted benzene derivatives and having the free valences at ring atoms are named as substituted phenylene radicals. Bivalent radicals derived from univalent polycyclic hydrocarbon radicals whose names end in “-yl” by removal of one hydrogen atom from the carbon atom with the free valence are named by adding “-idene” to the name of the corresponding univalent radical, e.g., a naphthyl group with two points of attachment is termed naphthylidene.

The term “heteroaryl” refers to a group selected from: 5- to 7-membered, e.g., 5- to 6-membered, aromatic, monocyclic rings comprising 1, 2, 3, or 4 heteroatoms selected from N, O, and S, with the remaining ring atoms being carbon; 8- to 12-membered bicyclic rings comprising 1, 2, 3, or 4 heteroatoms, selected from N, O, and S, with the remaining ring atoms being carbon and wherein at least one ring is aromatic and at least one heteroatom is present in the aromatic ring; and 11- to 14-membered tricyclic rings comprising 1, 2, 3, or 4 heteroatoms, selected from N, O, and S, with the remaining ring atoms being carbon and wherein at least one ring is aromatic and at least one heteroatom is present in an aromatic ring.

For example, the heteroaryl group may be a 5- to 7-membered heterocyclic aromatic ring fused to a 5- to 7-membered cycloalkyl ring. For such fused, bicyclic heteroaryl ring systems wherein only one of the rings comprises at least one heteroatom, the point of attachment may be at the heteroaromatic ring or at the cycloalkyl ring.

When the total number of S and O atoms in the heteroaryl group exceeds 1, those heteroatoms are not adjacent to one another. In some embodiments, the total number of S and O atoms in the heteroaryl group is not more than 2. In some embodiments, the total number of S and O atoms in the aromatic heterocycle is not more than 1.

Examples of the heteroaryl group include, but are not limited to, (as numbered from the linkage position assigned priority 1) pyridyl (such as 2-pyridyl, 3-pyridyl, or 4-pyridyl), cinnolinyl, pyrazinyl, 2,4-pyrimidinyl, 3,5-pyrimidinyl, 2,4-imidazolyl, imidazopyridinyl, isoxazolyl, oxazolyl, thiazolyl, isothiazolyl, thiadiazolyl, tetrazolyl, thienyl, triazinyl, benzothienyl, furyl, benzofuryl, benzoimidazolyl, indolyl, isoindolyl, indolinyl, phthalazinyl, pyrazinyl, pyridazinyl, pyrrolyl, triazolyl, quinolinyl, isoquinolinyl, pyrazolyl, pyrrolopyridinyl (such as 1H-pyrrolo[2,3-b]pyridin-5-yl), pyrazolopyridinyl (such as1H-pyrazolo[3,4-b]pyridin-5-yl), benzoxazolyl (such as benzo[d]oxazol-6-yl), pteridinyl, purinyl, 1-oxa-2,3-diazolyl, 1-oxa-2,4-diazolyl, 1-oxa-2,5-diazolyl, 1-oxa-3,4-diazolyl, 1-thia-2,3-diazolyl, 1-thia-2,4-diazolyl, 1-thia-2,5-diazolyl, 1-thia-3,4-diazolyl, furazanyl, benzofurazanyl, benzothiophenyl, benzothiazolyl, benzoxazolyl, quinazolinyl, quinoxalinyl, naphthyridinyl, furopyridinyl, benzothiazolyl (such as benzo[d]thiazol-6-yl), indazolyl (such as 1H-indazol-5-yl) and 5,6,7,8-tetrahydroisoquinolinyl.

The term “acyl” refers to a substituent group where a point of attachment in the substituent group is a carbonyl. Exemplary acyl groups include, but are not limited to, —C(═O) R′, —C(═O) NR′R″, or —C(═O) OR′, wherein R′ and R″ are independently selected from hydrogen, alkyl, alkenyl, alkynyl, heteroalkyl, heteroalkenyl, heteroalkynyl, cycloalkyl, aryl, heterocyclyl, or heteroaryl, any of which may be further substituted by one or more substituents.

Some of the compounds may exist with different points of attachment of hydrogen, referred to as “tautomers.” For example, compounds including carbonyl —CH₂C (O)-groups (keto forms) may undergo tautomerism to form hydroxyl-CH═C (OH)-groups (enol forms). Both keto and enol forms, individually as well as mixtures thereof, are also intended to be included where applicable.

The compounds, tautomers, solvates, or pharmaceutically acceptable salts of the disclosure may contain an asymmetric center and may thus exist as enantiomers. For example, where the compounds possess two or more asymmetric centers, they may additionally exist as diastereoisomers. Enantiomers and diastereoisomers fall within the broader class of stereoisomers. All such possible stereoisomers as substantially pure resolved enantiomers, racemic mixtures thereof, as well as mixtures of diastereoisomers are intended to be included in this disclosure. All stereoisomers of the compounds, tautomers, solvates, and pharmaceutically acceptable salts thereof are intended to be included. Unless specifically mentioned otherwise, reference to one isomer applies to any of the possible isomers. Whenever the isomeric composition is unspecified, all possible isomers are included.

Diastereomeric mixtures can be separated into their individual diastereoisomers on the basis of their physical chemical differences by methods well known to those skilled in the art, such as by chromatography and/or fractional crystallization. Enantiomers can be separated by converting the enantiomeric mixture into a diastereomeric mixture by reaction with an appropriate optically active compound (e.g., chiral auxiliary such as a chiral alcohol or Mosher's acid chloride), separating the diastereoisomers and converting (e.g., hydrolyzing) the individual diastereoisomers to the corresponding pure enantiomers. Enantiomers can also be separated by use of a chiral HPLC column.

A single stereoisomer, e.g., a substantially pure enantiomer, may be obtained by resolution of the racemic mixture using a method such as formation of diastereoisomers using optically active resolving agents. Racemic mixtures of chiral compounds of the disclosure can be separated and isolated by any suitable method, including: (1) formation of ionic, diastereomeric salts with chiral compounds and separation by fractional crystallization or other methods, (2) formation of diastereomeric compounds with chiral derivatizing reagents, separation of the diastereoisomers, and conversion to the pure stereoisomers, and (3) separation of the substantially pure or enriched stereoisomers directly under chiral conditions.

The term “substantially pure” in the context of stereoisomers means that the target stereoisomer contains no more than 35%, such as no more than 30%, further such as no more than 25%, even further such as no more than 20%, by weight of any other stereoisomer(s). In some embodiments, the term “substantially pure” means that the target stereoisomer contains no more than 10%, for example, no more than 5%, such as no more than 1%, by weight of any other stereoisomer(s).

Unless otherwise indicated, structures depicted herein are meant to include all isomeric forms of the structure, e.g., racemic mixtures, cis/trans isomers, geometric (or conformational) isomers, such as (Z) and (E) double bond isomers, and (Z) and (E) conformational isomers. Therefore, geometric and conformational mixtures of the compounds disclosed herein are within the scope of the disclosure. Unless otherwise stated, all tautomeric forms of the compounds of the disclosure are within the scope of the disclosure.

The disclosure provides pharmaceutically acceptable salts of the disclosed compounds, tautomers, solvates, and stereoisomers. A salt of a compound is formed between an acid and a basic group of the compound, such as an amino functional group, or a base and an acidic group of the compound, such as a carboxyl functional group.

The term “pharmaceutically acceptable,” as used herein, refers to a component that is, within the scope of sound medical judgment, suitable for use in contact with the tissues of humans and other mammals without undue toxicity, irritation, allergic response and the like, and are commensurate with a reasonable benefit/risk ratio. A “pharmaceutically acceptable salt” means any non-toxic salt that, upon administration to a recipient, is capable of providing, either directly or indirectly, a compound of this disclosure. “Pharmaceutically acceptable salts” include, but are not limited to salts with inorganic acids, selected, for example, from hydrochlorates, phosphates, diphosphates, hydrobromates, sulfates, sulfinates, and nitrates; as well as salts with organic acids, selected, for example, from malates, maleates, fumarates, tartrates, succinates, citrates, lactates, methanesulfonates, p-toluenesulfonates, 2-hydroxyethylsulfonates, benzoates, salicylates, stearates, alkanoates such as acetate, and salts with HOOC—(CH₂)n-COOH, wherein n is selected from 0 to 4. Similarly, examples of pharmaceutically acceptable cations include, but are not limited to, sodium, potassium, calcium, magnesium, aluminum, lithium, and ammonium. Suitable pharmaceutically acceptable salts are, for example, those disclosed in S. M. Berge, et al. J. Pharmaceutical Sciences, 1977, 66, pp. 1 to 19.

Acids commonly employed to form pharmaceutically acceptable salts include inorganic acids such as hydrogen bisulfide, hydrochloric acid, hydrobromic acid, hydroiodic acid, sulfuric acid and phosphoric acid, as well as organic acids such as para-toluenesulfonic acid, salicylic acid, tartaric acid, bitartaric acid, ascorbic acid, maleic acid, benzenesulfonic acid, fumaric acid, gluconic acid, glucuronic acid, formic acid, glutamic acid, methanesulfonic acid, ethanesulfonic acid, benzenesulfonic acid, lactic acid, oxalic acid, para-bromophenylsulfonic acid, carbonic acid, succinic acid, citric acid, benzoic acid, and acetic acid. Such pharmaceutically acceptable salts thus include sulfate, pyrosulfate, bisulfate, sulfite, bisulfite, phosphate, monohydrogenphosphate, dihydrogenphosphate, metaphosphate, pyrophosphate, chloride, bromide, iodide, acetate, propionate, decanoate (i.e., caprate), caprylate, acrylate, formate, isobutyrate, heptanoate, propiolate, oxalate, malonate, succinate, suberate, sebacate, fumarate, maleate, butyne-1,4-dioate, hexyne-1,6-dioate, benzoate, chlorobenzoate, methylbenzoate, dinitrobenzoate, hydroxybenzoate, methoxybenzoate, phthalate, terephthalate, sulfonate, xylene sulfonate, phenylacetate, phenylpropionate, phenylbutyrate, citrate, lactate, β-hydroxybutyrate, glycolate, tartrate, methanesulfonate, propanesulfonate, naphthalene-1-sulfonate, naphthalene-2-sulfonate, mandelate and other salts. In some embodiments, pharmaceutically acceptable acid addition salts include those formed with mineral acids such as hydrochloric acid and hydrobromic acid, and those formed with organic acids such as maleic acid.

Pharmaceutically acceptable salts derived from appropriate bases include alkali metal, alkaline earth metal, ammonium, and N⁺(C₁₋₄alkyl)₄ salts. This disclosure also envisions the quaternization of any basic nitrogen-containing groups of the compounds disclosed herein. Suitable non-limiting examples of alkali and alkaline earth metal salts include sodium, lithium, potassium, calcium, and magnesium salts. Further non-limiting examples of pharmaceutically acceptable salts include salts of ammonium, quaternary ammonium, and amine cations formed using counterions such as halide, hydroxide, carboxylate, sulfate, phosphate, nitrate, lower alkyl sulfonate and aryl sulfonate. Other suitable, non-limiting examples of pharmaceutically acceptable salts include besylate and glucosamine salts.

If a compound is obtained as an acid addition salt, the free base can be obtained by basifying a solution of the acid addition salt. Conversely, if the product is a free base, an addition salt, such as a pharmaceutically acceptable addition salt, may be produced by dissolving the free base in a suitable organic solvent and treating the solution with an acid, in accordance with conventional procedures for preparing acid addition salts from base compounds. Those skilled in the art will recognize various synthetic methodologies that may be used without undue experimentation to prepare non-toxic pharmaceutically acceptable addition salts.

The compounds, tautomers, solvates, stereoisomers, and pharmaceutically acceptable salts of the disclosure may also contain unnatural proportions of atomic isotopes at one or more of the atoms that constitute such compounds. For example, —CD₃, —CD₂H or —CDH₂ contains one or more deuteriums in place of hydrogen. For example, the compounds may be radiolabeled with radioactive isotopes, such as for example tritium (³H), iodine-125 (¹²⁵I) or carbon-14 (¹⁴C). All isotopic variations of the compounds of the disclosure, whether radioactive or not, are intended to be encompassed within the scope of the disclosure.

As used herein, “optionally substituted” is interchangeable with the phrase “substituted or unsubstituted.” In general, the term “substituted,” refers to the replacement of a hydrogen radical in a given structure with the radical of a specified substituent. Unless otherwise indicated, an “optionally substituted” group may have a substituent at each substitutable position of the group, and when more than one position in any given structure may be substituted with more than one substituent chosen from a specified group, the substituent may be either the same or different at every position. Combinations of substituents envisioned by this disclosure are those that result in the formation of stable or chemically feasible compounds.

In some embodiments, substituents are independently selected from optionally substituted heteroatom and optionally substituted, optionally hetero-, optionally cyclic C₁-C₁₈ hydrocarbyl, particularly wherein the optionally substituted, optionally hetero-, optionally cyclic C₁-C₁₈ hydrocarbyl is optionally-substituted, optionally hetero-, optionally cyclic alkyl, alkenyl or alkynyl, or optionally-substituted, optionally hetero-, aryl; and/or the optionally substituted heteroatom is halogen, optionally substituted hydroxyl (such as alkoxy, aryloxy), optionally substituted acyl (such as formyl, alkanoyl, carbamoyl, carboxyl, amido), optionally substituted amino (such as amino, alkylamino, dialkylamino, amido, sulfamidyl), optionally substituted thiol (such as mercapto, alkylthiol, aryl thiol), optionally substituted sulfinyl or sulfonyl (such as alkylsulfinyl, arylsulfinyl, alkyl sulfonyl, arylsulfonyl), nitro, or cyano.

In some embodiments, substituents are independently selected from: halogen, —R′, —OR′, ═O, —NR′, —N—OR′, —NR′R″, —SR′, —SiR′R″R″, —OC(═O)R′, —C(═O) R′, —CO₂R′, —C(O)NR′R″, —OC(═O) NR′R″, —NR″C(═O)R′, —NR′—C(═O)NR″R″, —NR′—SO₂NR″R″, —NR″CO₂R′, —NH—C(NH₂)—NH, —NR′C(NH₂)═NH, —NH—C(NH₂)═NR′, —S(O) R′, —SO₂R′, —SO₂NR′R″, —NR″SO₂R, —CN, —NO₂, —N₃, —CH(Ph)₂, perfluoro (C₁-C₄) alkoxy, and perfluoro (C₁-C₄) alkyl, in a number ranging from zero to three, with those groups having zero, one, or two substituents being particularly preferred. R′, R″ and R′″ each independently refer to hydrogen, unsubstituted C₁-C₈ alkyl and heteroalkyl, C₁-C₈ alkyl and heteroalkyl substituted with one to three halogens, unsubstituted aryl, aryl substituted with one to three halogens, unsubstituted alkyl, alkoxy, or thioalkoxy groups, or aryl-(C₁-C₄) alkyl groups. When R′ and R″ are attached to the same nitrogen atom, they can be combined with the nitrogen atom to form a 5-, 6- or 7-membered ring. Hence, —NR′R″ includes 1-pyrrolidinyl and 4-morpholinyl. When the aryl group is 1,2,3,4-tetrahydronaphthalenyl, it may be substituted with a substituted or unsubstituted C₃-C₇ spirocycloalkyl group. The C₃-C₇ spirocycloalkyl group may be substituted in the same manner as defined herein for “cycloalkyl.”

In some embodiments, substituents are selected from: halogen, —R′, —OR′, —O, —NR′R″, —SR′, —SiR′R″R″, —OC(═O) R′, —C(═O) R′, —CO₂R′, —C(═O)NR′R″, —OC(═O)NR′R″, —NR″C(═O)R′, —NR″CO₂R′, —NR′—SO₂NR″R″, —S(═O) R′, —SO₂R′, —SO₂NR′R″, —NR″SO₂R, —CN, —NO₂, perfluoro C₁-C₄ alkoxy and perfluoro C₁-C₄ alkyl, where R′ and R″ are as defined above.

In some embodiments, substituents are independently selected from substituted or unsubstituted heteroatom, substituted or unsubstituted, 0-3 heteroatom-containing C₁-C₆ alkyl (e.g., C₁-C₃ alkyl or C₁-C₂ alkyl), substituted or unsubstituted, 0-3 heteroatom-containing C₂-C₆ alkenyl (e.g., C₂-C₄ alkenyl), substituted or unsubstituted, 0-3 heteroatom-containing C₂-C₆ alkynyl (e.g., C₂-C₄ alkynyl), or substituted or unsubstituted, 0-3 heteroatom-containing C₆-C₁₄ aryl (e.g., C₅-C₆ aryl), wherein each heteroatom is independently oxygen, phosphorus, sulfur, or nitrogen.

In some embodiments, substituents are independently selected from aldehyde, aldimine, alkanoyloxy, alkoxy, alkoxycarbonyl, alkyloxy, alkyl, alkenyl, alkynyl, amine, azo, halogen, carbamoyl, carbonyl, carboxamido, carboxyl, cyanyl, ester, haloformyl, hydroperoxyl, hydroxyl, iminc, isocyanide, iscyante, N-tert-butoxycarbonyl, nitrate, nitrile, nitrite, nitro, nitroso, phosphate, phosphono, sulfide, sulfonyl, sulfo, sulfhydryl, thiol, thiocyanyl, trifluoromethyl, and trifluromethyl ether (OCF₃) groups.

Preferred substituents are disclosed herein and exemplified in the tables, structures, examples, and claims, and may be applied across different compounds of this disclosure. For example, substituents of a given compound may be combinatorically used with other compounds.

It may be advantageous to separate reaction products from one another and/or from starting materials. The desired products of each step or series of steps are separated and/or purified (hereinafter separated) to the desired degree of homogeneity by the techniques common in the art. Typically such separations involve multiphase extraction, crystallization from a solvent or solvent mixture, distillation, sublimation, or chromatography. Chromatography can involve any number of methods including, for example, reverse-phase and normal phase; size exclusion; ion exchange; high, medium and low pressure liquid chromatography methods and apparatus; small scale analytical; simulated moving bed (“SMB”) and preparative thin or thick layer chromatography, as well as techniques of small scale thin layer and flash chromatography. One skilled in the art may apply such techniques to achieve a desired separation.

Non-limiting examples of suitable solvents that may be used in this disclosure include water, methanol (MeOH), ethanol (EtOH), dichloromethane or methylene chloride (CH₂Cl₂), toluene, acetonitrile (MeCN), dimethylformamide (DMF), dimethyl sulfoxide (DMSO), methyl acetate (MeOAc), ethyl acetate (EtOAc), heptanes, isopropyl acetate (IPAc), tert-butyl acetate (1-BuOAc), isopropyl alcohol (IPA), tetrahydrofuran (THF), 2-methyl tetrahydrofuran (2-Me THF), methyl ethyl ketone (MEK), tert-butanol, diethyl ether (Et₂O), methyl-tert-butyl ether (MTBE), 1,4-dioxane, and N-methyl pyrrolidone (NMP).

Non-limiting examples of suitable bases that may be used in this disclosure include 1,8-diazabicyclo [5.4.0]undec-7-ene (DBU), potassium tert-butoxide (KOtBu), potassium carbonate (K₂CO₃), N-methylmorpholine (NMM), triethylamine (Et₃N; TEA), diisopropyl-ethyl amine (i-Pr₂EtN; DIPEA), pyridine, potassium hydroxide (KOH), sodium hydroxide (NaOH), lithium hydroxide (LiOH), and sodium methoxide (NaOMe; NaOCH₃).

The term “subject” refers to an animal including a human.

The term “therapeutically effective amount” refers to the amount of a compound that produces a desired effect for which it is administered (e.g., improvement in a disease or condition, lessening the severity of a disease or condition, and/or reducing progression of a disease or condition, e.g., a disease or condition selected from neuropathy, stroke, neurodegenerative disease (e.g., Alzheimer's disease), Parkinson's Disease, amyotrophic lateral sclerosis (AML), multiple sclerosis, Huntington's Disease, dementia with Lewy bodies, Friedreich's ataxia, hair follicle morphogenesis, diabetes, sepsis, transplant rejection, periventricular leukomalacia, ischemia reperfusion injury, blood coagulation, myocardial infarction, and kidney dysfunction such as acute kidney failure. The disease or condition may be involved with dysregulated, e.g., abnormal, ferroptosis. The exact amount of a therapeutically effective amount will depend on the purpose of the treatment and will be ascertainable by one skilled in the art using known techniques (see, e.g., Lloyd (1999), The Art, Science and Technology of Pharmaceutical Compounding).

As used herein, the term “treatment” and its cognates refer to slowing or stopping disease progression. “Treatment” and its cognates as used herein include, but are not limited to the following: complete or partial remission, curing a disease or condition or a symptom thereof, lower risk of a disease or condition, e.g., neuropathy, stroke, neurodegenerative disease (e.g., Alzheimer's disease), Parkinson's Disease, amyotrophic lateral sclerosis (AML), multiple sclerosis, Huntington's Disease, dementia with Lewy bodies, Friedreich's ataxia, hair follicle morphogenesis, diabetes, sepsis, transplant rejection, periventricular leukomalacia, ischemia reperfusion injury, blood coagulation, myocardial infarction, and kidney dysfunction such as acute kidney failure. The disease or condition may be involved with dysregulated, e.g., abnormal, ferroptosis. Improvements in or lessening the severity of any of these symptoms can be assessed according to methods and techniques known in the art. The terms “about” and “approximately,” when used in connection with a number such as a percentage include the number as specified, and a range of the number (e.g., a range of percentages, for example, a range of ±10% with respect to a specific point value) that is recognized by one of ordinary skill in the art.

### II. Compounds and Compositions

In a first embodiment, a compound of this disclosure is a compound of the following structural Formula I:

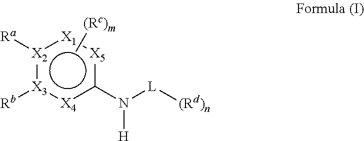

a tautomer thereof, a solvate or stereoisomer of the compound or the tautomer, or a pharmaceutically acceptable salt of the foregoing, wherein:
L is cycloalkyl, heterocyclyl, or C,
X₁, X₂, X₃, X₄, and X₅ are each independently C or N;
R^{a} is absent or selected from H, halogen, CN, optionally substituted heteroatom, optionally substituted alkyl, optionally substituted alkenyl, optionally substituted alkynyl, optionally substituted acyl, optionally substituted cycloalkyl, optionally substituted heterocyclyl, optionally substituted aryl, and optionally substituted heteroaryl;
R^{b} is absent or selected from H, halogen, CN, optionally substituted heteroatom, optionally substituted alkyl, optionally substituted cycloalkyl, and optionally substituted heterocyclyl;
R^{a} and R^{b} may join together to form an optionally substituted 3- to 10-membered carbocyclic, heterocyclic, aromatic, or heteroaromatic ring;
R^{c}, for each occurrence, is independently selected from halogen, optionally substituted heteroatom, and optionally substituted alkyl;
R^{b} and one of R^{c} may join together to form an optionally substituted 3- to 10-membered carbocyclic, heterocyclic, aromatic, or heteroaromatic ring;
R^{d}, for each occurrence, is independently selected from halogen, CN, optionally substituted heteroatom, optionally substituted alkyl, optionally substituted alkenyl, optionally substituted alkynyl, optionally substituted acyl, optionally substituted cycloalkyl, optionally substituted heterocyclyl, optionally substituted aryl, and optionally substituted heteroaryl; and m and n are each an integer independently selected from 0, 1, 2, and 3.

Combinations of substituents as disclosed herein are those that result in the formation of stable or chemically feasible compounds. For abbreviation or according to common practice, certain hydrogen atoms attached to a certain atom (e.g., a carbon atom C or a nitrogen atom N) are not specifically spelled out in a chemical structure, formula, or notation; hydrogen atoms are deemed to be present to the extent the valences of the certain atom (e.g., C or N) are completed.

In a second embodiment, a compound of the disclosure is a compound of the following structural Formula IIa:

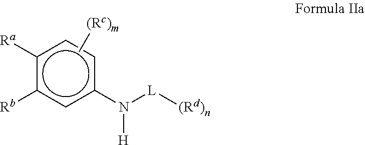

a tautomer thereof, a solvate or stereoisomer of the compound or the tautomer, or a pharmaceutically acceptable salt of the foregoing; and all other variables not specifically defined herein are as defined in any one of the preceding embodiments.

In a third embodiment, a compound of the disclosure is a compound of the following structural Formula IIb:

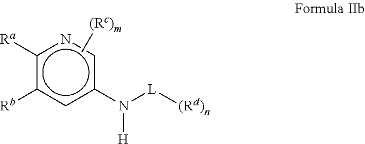

a tautomer thereof, a solvate or stereoisomer of the compound or the tautomer, or a pharmaceutically acceptable salt of the foregoing; and all other variables not specifically defined herein are as defined in any one of the preceding embodiments.

In a fourth embodiment, a compound of the disclosure is a compound of the following structural Formula IIc:

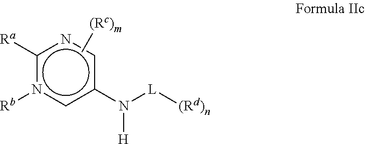

a tautomer thereof, a solvate or stereoisomer of the compound or the tautomer, or a pharmaceutically acceptable salt of the foregoing; and all other variables not specifically defined herein are as defined in any one of the preceding embodiments.

In a fifth embodiment, a compound of the disclosure is a compound of the following structural Formula IId:

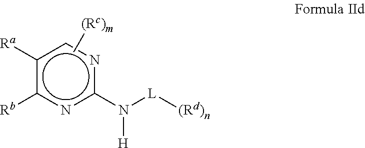

a tautomer thereof, a solvate or stereoisomer of the compound or the tautomer, or a pharmaceutically acceptable salt of the foregoing; and all other variables not specifically defined herein are as defined in any one of the preceding embodiments.

In a sixth embodiment, a compound of the disclosure is a compound of the following structural Formula Ile:

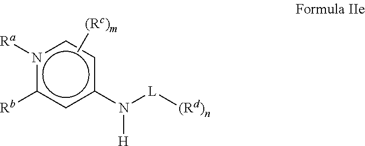

a tautomer thereof, a solvate or stereoisomer of the compound or the tautomer, or a pharmaceutically acceptable salt of the foregoing; and all other variables not specifically defined herein are as defined in any one of the preceding embodiments.

In a seventh embodiment, a compound of the disclosure is a compound of the following structural Formula IIf:

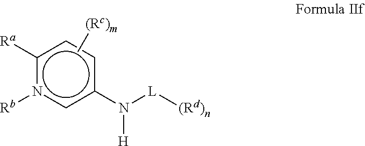

a tautomer thereof, a solvate or stereoisomer of the compound or the tautomer, or a pharmaceutically acceptable salt of the foregoing; and all other variables not specifically defined herein are as defined in any one of the preceding embodiments.

In an eighth embodiment, a compound of the disclosure is a compound of the following structural Formula IIg:

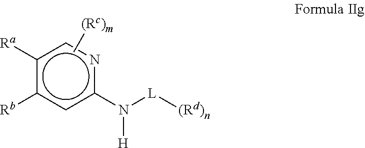

a tautomer thereof, a solvate or stereoisomer of the compound or the tautomer, or a pharmaceutically acceptable salt of the foregoing; and all other variables not specifically defined herein are as defined in any one of the preceding embodiments.

In a ninth embodiment, a compound of the disclosure is a compound of the following structural Formula IIh:

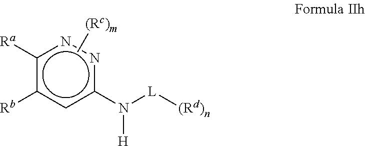

a tautomer thereof, a solvate or stereoisomer of the compound or the tautomer, or a pharmaceutically acceptable salt of the foregoing; and all other variables not specifically defined herein are as defined in any one of the preceding embodiments.

In a tenth embodiment, a compound of the disclosure is a compound of the following structural Formula IIi:

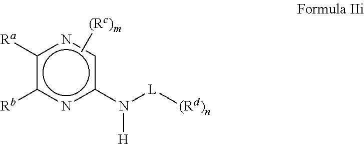

a tautomer thereof, a solvate or stereoisomer of the compound or the tautomer, or a pharmaceutically acceptable salt of the foregoing; and all other variables not specifically defined herein are as defined in any one of the preceding embodiments.

In an eleventh embodiment, a compound of the disclosure is a compound of the following structural Formula IIj:

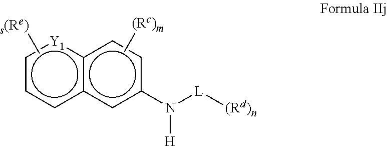

a tautomer thereof, a solvate or stereoisomer of the compound or the tautomer, or a pharmaceutically acceptable salt of the foregoing; and all other variables not specifically defined herein are as defined in any one of the preceding embodiments.

In a twelfth embodiment, a compound of the disclosure is a compound of the following structural Formula IIIa:

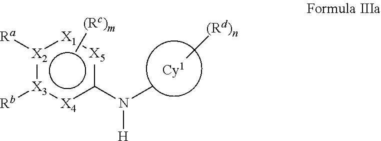

a tautomer thereof, a solvate or stereoisomer of the compound or the tautomer, or a pharmaceutically acceptable salt of the foregoing; wherein Cy¹ is 3- to 10-membered cycloalkyl or 3- to 10-membered heterocyclyl; and all other variables not specifically defined herein are as defined in any one of the preceding embodiments.

In a thirteenth embodiment, a compound of the disclosure is a compound of the following structural Formula IIIb:

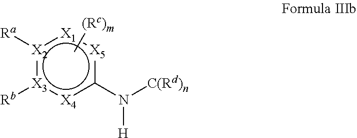

a tautomer thereof, a solvate or stereoisomer of the compound or the tautomer, or a pharmaceutically acceptable salt of the foregoing; and all other variables not specifically defined herein are as defined in any one of the preceding embodiments.

In a fourteenth embodiment, a compound of the disclosure is a compound of the following structural Formula IVa:

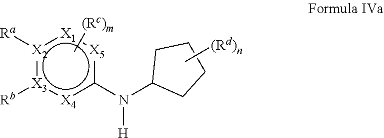

a tautomer thereof, a solvate or stereoisomer of the compound or the tautomer, or a pharmaceutically acceptable salt of the foregoing; and all other variables not specifically defined herein are as defined in any one of the preceding embodiments.

In a fifteenth embodiment, a compound of the disclosure is a compound of the following structural Formula IVb:

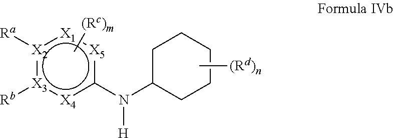

a tautomer thereof, a solvate or stereoisomer of the compound or the tautomer, or a pharmaceutically acceptable salt of the foregoing; and all other variables not specifically defined herein are as defined in any one of the preceding embodiments.

In a sixteenth embodiment, a compound of the disclosure is a compound of the following structural Formula IVc:

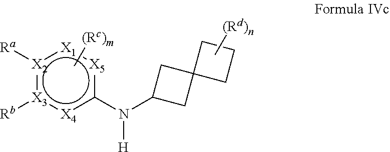

a tautomer thereof, a solvate or stereoisomer of the compound or the tautomer, or a pharmaceutically acceptable salt of the foregoing; and all other variables not specifically defined herein are as defined in any one of the preceding embodiments.

In a seventeenth embodiment, a compound of the disclosure is a compound of the following structural Formula IVd:

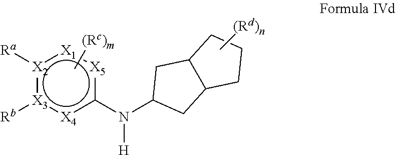

a tautomer thereof, a solvate or stereoisomer of the compound or the tautomer, or a pharmaceutically acceptable salt of the foregoing; and all other variables not specifically defined herein are as defined in any one of the preceding embodiments.

In an eighteenth embodiment, a compound of the disclosure is a compound of the following structural Formula IVe:

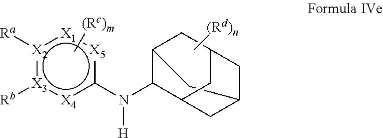

a tautomer thereof, a solvate or stereoisomer of the compound or the tautomer, or a pharmaceutically acceptable salt of the foregoing; and all other variables not specifically defined herein are as defined in any one of the preceding embodiments.

In a nineteenth embodiment, a compound of the disclosure is a compound of the following structural Formula IVf:

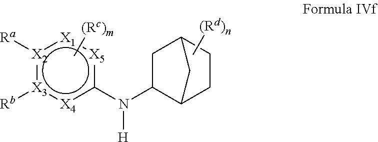

a tautomer thereof, a solvate or stereoisomer of the compound or the tautomer, or a pharmaceutically acceptable salt of the foregoing; and all other variables not specifically defined herein are as defined in any one of the preceding embodiments.

In a twentieth embodiment, a compound of the disclosure is a compound of the following structural Formula Va:

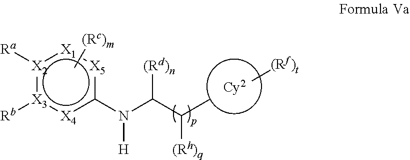

a tautomer thereof, a solvate or stereoisomer of the compound or the tautomer, or a pharmaceutically acceptable salt of the foregoing, wherein Cy² is 3- to 10-membered cycloalkyl or 3- to 10-membered heterocyclyl; R^{f}, for each occurrence, is independently selected from halogen, CN, optionally substituted heteroatom, optionally substituted alkyl, optionally substituted alkenyl, optionally substituted alkynyl, optionally substituted acyl, optionally substituted cycloalkyl, optionally substituted heterocyclyl, optionally substituted aryl, and optionally substituted heteroaryl; R″, for each occurrence, is independently selected from halogen, CN, and optionally substituted C₁ to C₆ alkyl; n is an integer selected from 0, 1, and 2; p is an integer selected from 0, 1, 2, 3, 4, and 5; q is an integer selected from 0, 1, and 2; t is an integer independently selected from 0, 1, 2, and 3; and all other variables not specifically defined herein are as defined in any one of the preceding embodiments.

In a twenty-first embodiment, in a compound, tautomer, a solvate or stereoisomer of the compound or the tautomer, or pharmaceutically acceptable salt of this disclosure, n is 0, q is 0, and p is 0, 1, 2, and 3 each occurrence; and all other variables not specifically defined herein are as defined in any one of the preceding embodiments.

In a twenty-second embodiment, a compound of the disclosure is a compound of the following structural Formula VIa:

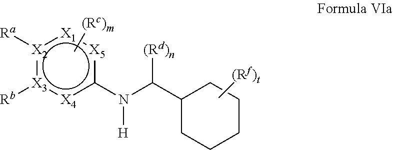

a tautomer thereof, a solvate or stereoisomer of the compound or the tautomer, or a pharmaceutically acceptable salt of the foregoing; and all other variables not specifically defined herein are as defined in any one of the preceding embodiments.

In a twenty-third embodiment, a compound of the disclosure is a compound of the following structural Formulae VIIa-VIIe:

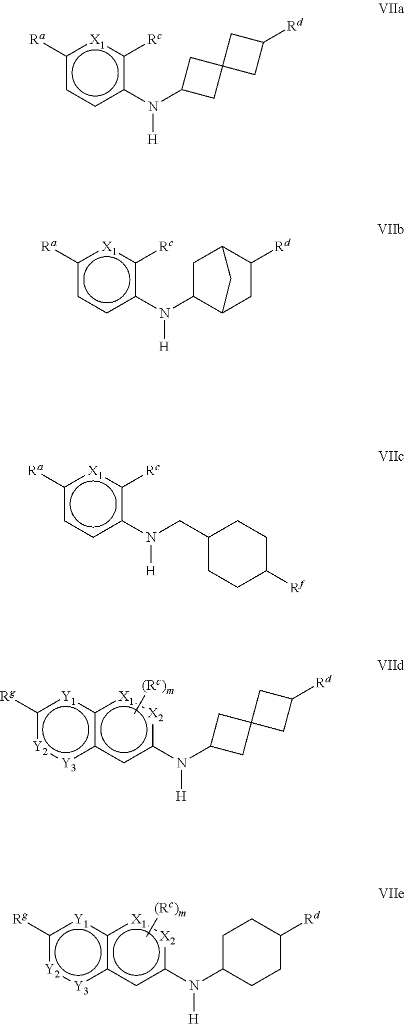

a tautomer thereof, a solvate or stereoisomer of the compound or the tautomer, or a pharmaceutically acceptable salt of the foregoing, wherein:
X₁ and X₂ are independently C or N;
Y₁, Y₂, and Y₃ are independently C or N;
R^{a} is selected from

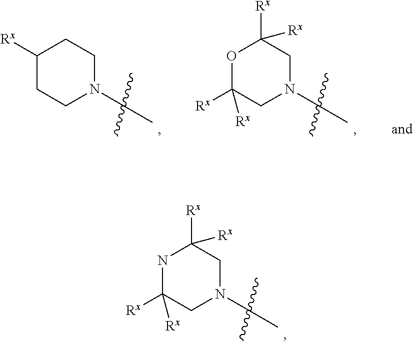

wherein R^{x}, for each occurrence, is independently selected from H, CF₃, —CH₃, and —CH₂CH₃;
R^{c} is selected from halogen, C₁ to C₂ alkyl, and C to C₂ alkoxy;
R^{d} is selected from NH₂ and —C(═O)NH₂;
R^{f} is selected from NH₂ and —C(═O) NH₂; and
m is 0 or 1;
and all other variables not specifically defined herein are as defined in any one of the preceding embodiments.

In a twenty-fourth embodiment, in a compound, tautomer, a solvate or stereoisomer of the compound or the tautomer, or pharmaceutically acceptable salt of this disclosure, R^{e}, for each occurrence, is independently selected from 5- to 6-membered cycloalkyl and 5- to 6-membered heterocyclyl; all other variables not specifically defined herein are as defined in any one of the preceding embodiments.

In a twenty-fifth embodiment, in a compound, tautomer, a solvate or stereoisomer of the compound or the tautomer, or pharmaceutically acceptable salt of this disclosure, R^{e} is selected from

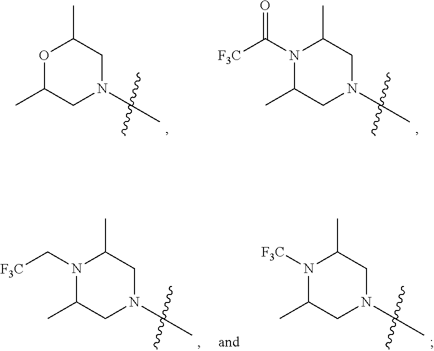

all other variables not specifically defined herein are as defined in any one of the preceding embodiments.

In a twenty-sixth embodiment, in a compound, tautomer, a solvate or stereoisomer of the compound or the tautomer, or pharmaceutically acceptable salt of this disclosure, R^{f}, for each occurrence, is independently selected from halogen, cyano, C₁-C₁₀ alkyl, phenyl, 5- to 7-membered heteroaryl, phenyl, 3- to 10-membered heterocyclyl, C₃-C₁₀ cycloalkyl, —C(═O)R^{s}, C₂-C₁₀ alkenyl, ═O, ═NR^{p}; —C(═O)OR^{s}, —C(═O)NR^{p}R^{q}, —NR^{p}R^{q}, and —NR^{p}C(═O)R^{s}, wherein the C₁-C₁₀ alkyl and C₂-C₁₀ alkenyl of R^{f} are each optionally substituted with 1 to 3 groups selected from 5- to 7-membered heteroaryl, C₃-C₁₀ cycloalkyl, 3- to 10-membered heterocyclyl, —OR^{s}, —C(═O)NR^{p}R^{q}, —NR^{p}C(═O)NR^{q}R^{r}, and —NR^{p}R^{q};
the 3- to 10-membered heterocyclyl and C₃-C₁₀ cycloalkyl of R^{f} and the 3- to 10-membered heterocyclyl and C₃-C₆ cycloalkyl of the C₁-C₁₀ alkyl and C₂-C₁₀ alkenyl of R^{f}, are each optionally substituted with 1 to 3 groups selected from ═O and C₁-C₆ alkyl optionally substituted with 1 to 3 groups selected from halogen and —OR^{s}, the phenyl and 5- to 7-membered heteroaryl of R′ and the 5- to 7-membered heteroaryl of the C₁-C₁₀ alkyl and C₂-C₁₀ alkenyl of R^{f} are each optionally substituted with 1 to 3 groups selected from C₁-C₆ alkyl optionally substituted with 1 to 3 groups selected from halogen and —OR^{s};
R^{p}, R^{q}, and R^{r}, for each occurrence, are independently selected from hydrogen, C₃-C₁₀ cycloalkyl, and C₁-C₁₀ alkyl, wherein the C₃-C₁₀ cycloalkyl and C₁-C₁₀ alkyl of R^{p}, R^{q}, and R^{r} are each optionally substituted with 1 to 3 groups selected from halogen, —OR^{s} and —C(═O)OR^{s};
R^{s}, for each occurrence, is independently selected from H, C₃-C₁₀ cycloalkyl, 3- to 10-membered heterocyclyl, and C₁-C₁₀ alkyl optionally substituted with 1 to 3 groups selected from halogen, C₃-C₁₀ cycloalkyl, 3- to 10-membered heterocyclyl, and C₁-C₆ alkoxy, wherein
the C₃-C₁₀ cycloalkyl and 3- to 10-membered heterocyclyl of R^{s} and the C₃-C₁₀ cycloalkyl and 3- to 10-membered heterocyclyl of the C₁-C₁₀ alkyl of R^{s} are each optionally substituted with 1 to 3 groups of C₁-C₆ alkyl optionally substituted by halogen, and the C₁-C₆ alkoxy of the C₁-C₁₀ alkyl of R^{s} is optionally substituted with 1 to 3 groups of halogen; all other variables not specifically defined herein are as defined in any one of the preceding embodiments.

In a twenty-seventh embodiment, in a compound, tautomer, a solvate or stereoisomer of the compound or the tautomer, or pharmaceutically acceptable salt of this disclosure, R^{f}, for each occurrence, is selected from H, methyl, NH₂, —NH(CH₂)₂OH, —CH₂NH₂,

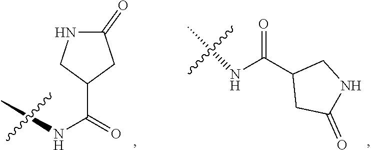

—NHC(═O) NH₂, —NHCH₃, —C(═O)NH₂, and —NHBoc.

In a twenty-eighth embodiment, in a compound, tautomer, a solvate or stereoisomer of the compound or the tautomer, or pharmaceutically acceptable salt of this disclosure, R^{a} is absent or selected from hydrogen, halogen, cyano, C₁-C₁₀ alkyl, C₂-C₁₀ alkenyl, C₂-C₁₀ alkynyl, C₃-C₁₀ cycloalkyl, 3- to 10-membered heterocyclyl, phenyl, 5- to 7-membered heteroaryl, —C(—O)R^{s}, —C(═O)OR^{s}, —NR^{p}R^{q}, —OR^{s}, wherein the C₃-C₁₀ cycloalkyl, 3- to 10-membered heterocyclyl, phenyl, and 5- to 7-membered heteroaryl of R^{a} are each optionally substituted with 1-3 groups selected from halogen, OR^{s}, and C₁-C₁₀ alkyl optionally substituted with 1 to 3 groups of halogen;
the C₁-C₁₀ alkyl, C₂-C₁₀ alkenyl, and C₂-C₁₀ alkynyl of R^{a} are each optionally substituted with 1 to 3 groups selected from halogen, 3- to 10-membered heterocyclyl, and —OR^{s};
R^{p} and R^{q}, for each occurrence, are independently selected from hydrogen, C₃-C₁₀ cycloalkyl, and C₁-C₁₀ alkyl, wherein the C₃-C₁₀ cycloalkyl and C₁-C₁₀ alkyl of R^{p} and R^{q} are each optionally substituted with 1 to 3 groups selected from halogen, —OR^{s} and —C(═O)OR^{s};
R^{s}, for each occurrence, is selected from H, C₃-C₁₀ cycloalkyl, 3- to 10-membered heterocyclyl, 5- to 6-membered aryl, and C₁-C₁₀ alkyl optionally substituted with 1 to 3 groups selected from halogen, C₃-C₁₀ cycloalkyl, 3- to 10-membered heterocyclyl, and C₁-C₆ alkoxy, wherein the C₃-C₁₀ cycloalkyl and 3- to 10-membered heterocyclyl of R^{s} and the C₃-C₁₀ cycloalkyl and 3- to 10-membered heterocyclyl of the C₁-C₁₀ alkyl of R^{s} are each optionally substituted with 1 to 3 groups of C₁-C₆ alkyl optionally substituted with halogen, and the C₁-C₆ alkoxy of the C₁-C₁₀ alkyl of R^{s} is optionally substituted with 1 to 3 groups of halogen; and
all other variables not specifically defined herein are as defined in any one of the preceding embodiments.

In a twenty-ninth embodiment, in a compound, tautomer, a solvate or stereoisomer of the compound or the tautomer, or pharmaceutically acceptable salt of this disclosure, R^{a} is absent or selected from H, F, Cl, Br, methyl,

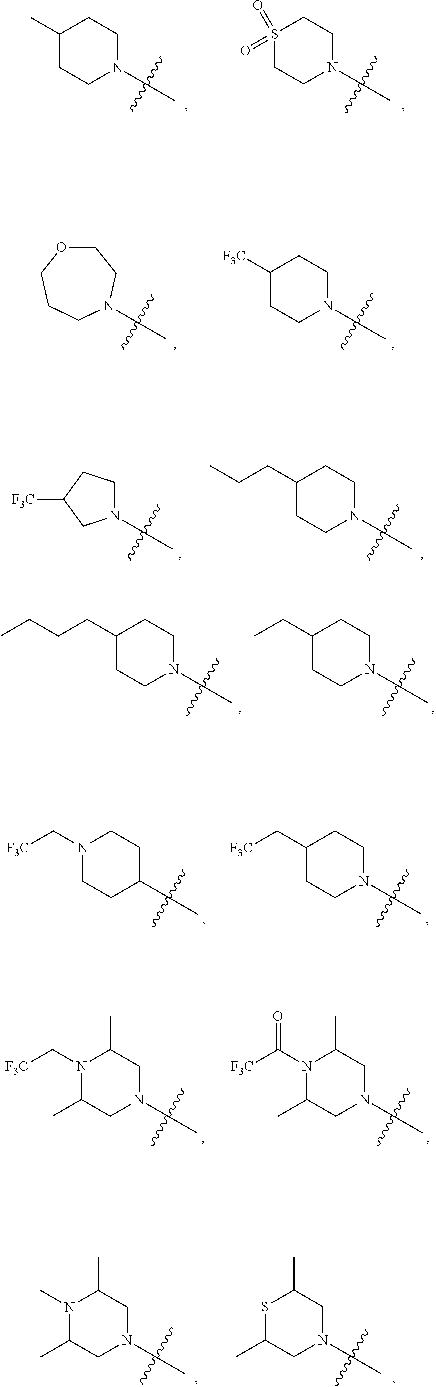

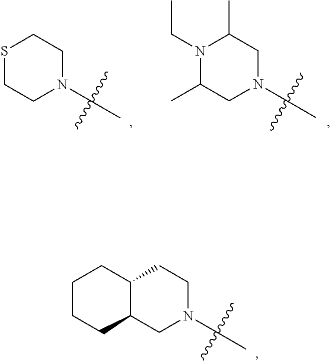 [SMILES: *N1CCSCC1 ; *N1CC(C)N(CC)C(C)C1 ; *N1CC[C@H]2CCCC[C@@H]2C1]

—CH (CH₂CH₃)₂, —CH (CH₃)₂, —OCH(CH₃)₂, —CH₂CH₃, —CH₂CH₂CH₃, —NHCH₂CF₃, —N(CH₃)CH₂CF₃, —O(CH₂)₃CH₃, —O(CH₂),CH₃, —(CH₂)₂CH₃, —(CH₂)₄CH₃, —(CH₂)₃CH₃,

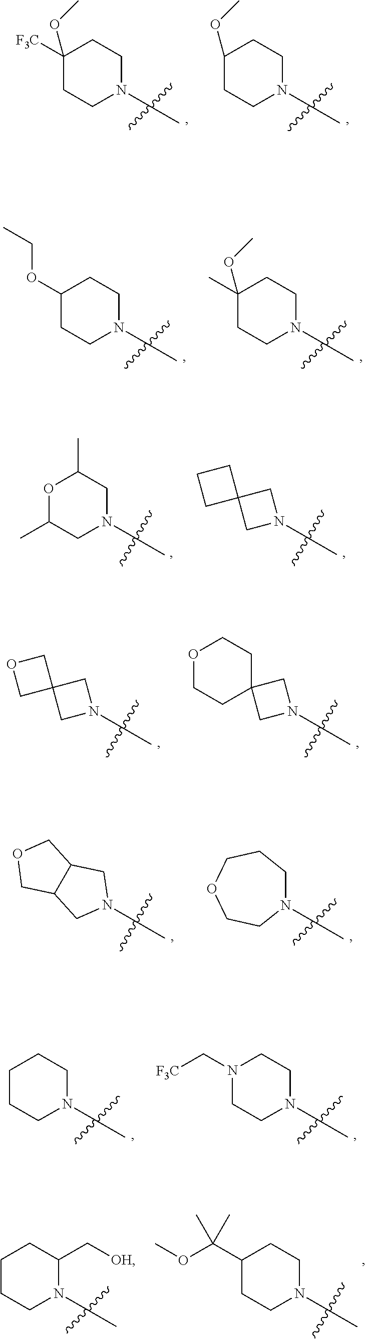

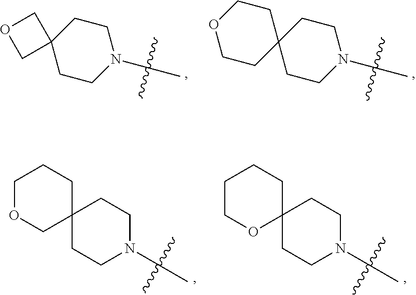 [SMILES: *N1CCC2(CC1)COC2 ; *N1CCC2(CCOCC2)CC1 ; *N1CCC2(CCCOC2)CC1 ; *N1CCC2(CCCCO2)CC1]

—N(CH₂CH₃)(CH₂)₄CH₃, —N(CH₂CH₃)(CH₂)₃OCH₃,

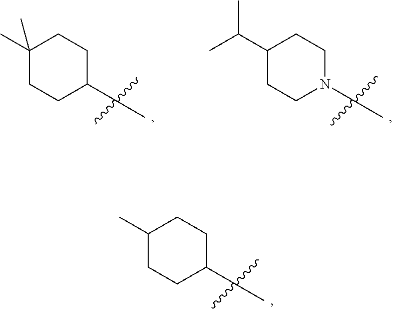

—C(CH₃)₂CH₂CH₃, —C(CH₃)₂CH₂CH₂CH₃, —C(CH₃)₂CH₂OCH₂CH₃, —C(CH₃)₃,

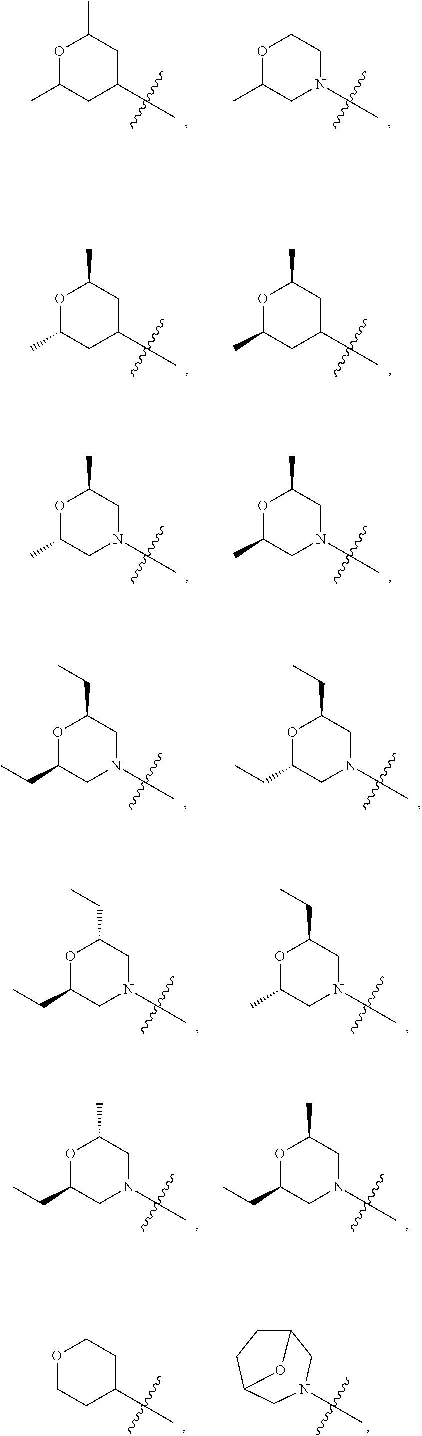

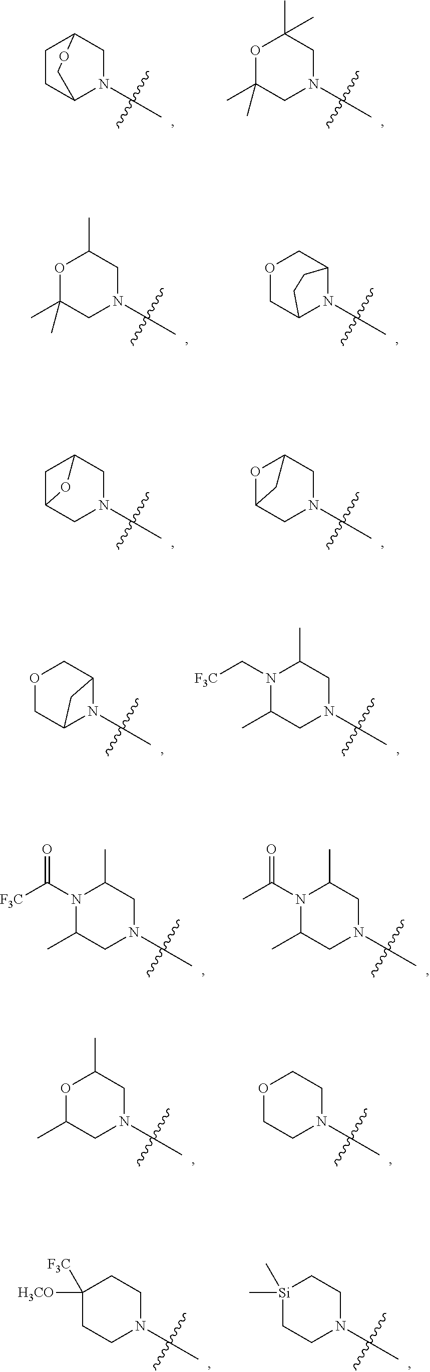

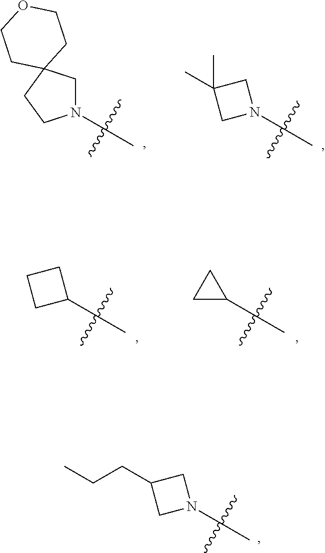

—N(CH₃)₂, —O(CH₂)₂OCH₂CH₃, —OCH₂CH₃, —(CH₂)₂OCH₃, —(CH₂)₃OCH₂CH₃,

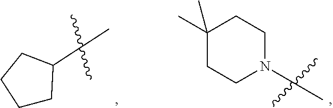

—CF₃, —CH₂CF₃, —N(CH₂CH₃)₂,

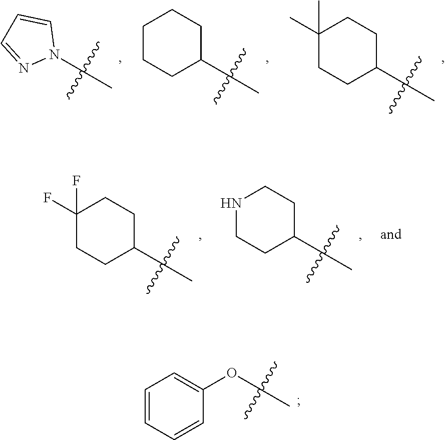

all other variables not specifically defined herein are as defined in any one of the preceding embodiments. In a thirtieth embodiment, in a compound, tautomer, a solvate or stereoisomer of the compound or the tautomer, or pharmaceutically acceptable salt of this disclosure, R^{a} is selected from

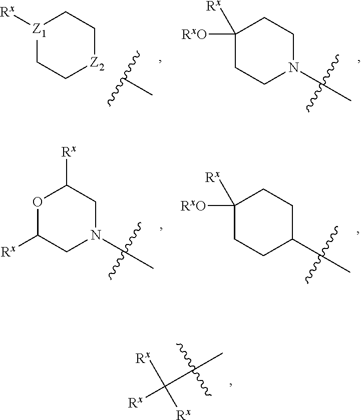

—OR^{x}, and —CH₂R^{x}, wherein Z₁ and Z₂ are independently selected from C and N, R^{x}, for each occurrence, is independently selected from C₁ to C₄ alkyl optionally substituted with 1 to 3 groups of halogen all other variables not specifically defined herein are as defined in any one of the preceding embodiments.

In a thirty-first embodiment, in a compound, tautomer, a solvate or stereoisomer of the compound or the tautomer, or pharmaceutically acceptable salt of this disclosure, R^{b} is absent or selected from H, halogen, C₁ to C₆ alkyl, —NR^{p}R^{q}, CN, C₁-C₆ alkoxy, and 5- to 6-membered heterocyclyl optionally substituted with C₁-C₃ alkyl optionally substituted with 1 to 3 groups selected from halogen, wherein R^{p} and R^{q} are each selected from C₁ to C₆ alkyl, and wherein the C₁-C₆ alkyl and C₁-C₆ alkoxy of R^{b} and the C₁-C₆ alkyl of R^{p} and R^{q} are each optionally substituted with 1 to 3 groups selected from halogen; all other variables not specifically defined herein are as defined in any one of the preceding embodiments.

In a thirty-second embodiment, in a compound, tautomer, a solvate or stereoisomer of the compound or the tautomer, or pharmaceutically acceptable salt of this disclosure, R^{b} is absent or H, methyl, ethyl, —O(CH₂)₄OCH₃, —O(CH₂)₃OCH₃, —O(CH₂)₂OCH₃, —O(CH₂)₂NH₂, —(CH₂)₅CH₃, —O(CH₂)₅CH₃, —(CH₂)₂OCH₃, —O(CH₂)₂OH, F, —OCH₃, —CF₃, Cl, CN, —C(CH₃)₃, and

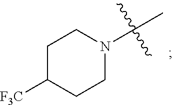 [SMILES: *N1CCC(C(F)(F)F)CC1]

all other variables not specifically defined herein are as defined in any one of the preceding embodiments.

In a thirty-third embodiment, in a compound, tautomer, a solvate or stereoisomer of the compound or the tautomer, or pharmaceutically acceptable salt of this disclosure, R^{b} is absent or selected from halogen, C₁-C₂ alkyl, and C₁-C₂ alkoxy; all other variables not specifically defined herein are as defined in any one of the preceding embodiments.

In a thirty-fourth embodiment, in a compound, tautomer, a solvate or stereoisomer of the compound or the tautomer, or pharmaceutically acceptable salt of this disclosure, R^{c}, for each occurrence, is independently selected from halogen, C₁-C₆ alkyl, —NR^{p}R^{q}, CN, and C₁-C₆ alkoxy, wherein R^{p} and R^{q} are each selected from C₁ to C₆ alkyl, and wherein the C₁-C₆ alkyl and C₁-C₆ alkoxy of R^{b} and the C₁-C₆ alkyl of R^{p} and R^{q} are each optionally substituted with 1 to 3 groups selected from halogen; all other variables not specifically defined herein are as defined in any one of the preceding embodiments.

In a thirty-fifth embodiment, in a compound, tautomer, a solvate or stereoisomer of the compound or the tautomer, or pharmaceutically acceptable salt of this disclosure, R^{c} is selected from methyl, ethyl, —O(CH₂)₄OCH₃, —O(CH₂)₃OCH₃, —O(CH₂)₂OCH₃, —O(CH₂)₂NH₂, —(CH₂)₅CH₃, —O(CH₂)₅CH₃, —(CH₂)₂OCH₃, —O(CH₂)₂OH, F, —OCH₃, —CF₃, Cl, CN,

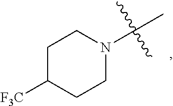 [SMILES: *N1CCC(C(F)(F)F)CC1]

and —C(CH₃)₃; all other variables not specifically defined herein are as defined in any one of the preceding embodiments.

In a thirty-sixth embodiment, in a compound, tautomer, a solvate or stereoisomer of the compound or the tautomer, or pharmaceutically acceptable salt of this disclosure, R^{c} is selected from halogen, C₁-C₂ alkyl, and C₁-C₂ alkoxy; all other variables not specifically defined herein are as defined in any one of the preceding embodiments.

In a thirty-seventh embodiment, in a compound, tautomer, a solvate or stereoisomer of the compound or the tautomer, or pharmaceutically acceptable salt of this disclosure, R^{d}, for each occurrence, is absent or independently selected from halogen, cyano, C₁-C₁₀ alkyl, phenyl, 5- to 7-membered heteroaryl, phenyl, 3- to 10-membered heterocyclyl, C₃-C₁₀ cycloalkyl, —C(═O) R^{s}, C₂-C₁₀ alkenyl, ═O, ═NR^{p}; —C(═O) OR^{s}, —C(═O) NR^{p}R^{q}, —NR^{p}R^{q}, and —NR^{p}C(—O)R^{s}, wherein
the C₁-C₁₀ alkyl and C₂-C₁₀ alkenyl of R^{d} are each optionally substituted with 1 to 3 groups selected from 5- to 7-membered heteroaryl, C₃-C₁₀ cycloalkyl, 3- to 10-membered heterocyclyl, —OR^{s}, —C(═O)NR^{p}R^{q}, —NR^{p}C(═O)NR^{q}R^{r}, and —NR^{p}R^{q};
the 3- to 10-membered heterocyclyl and C₃-C₁₀ cycloalkyl of R^{d} and the 3- to 10-membered heterocyclyl and C₃-C₆ cycloalkyl of the C₁-C₁₀ alkyl and C₂-C₁₀ alkenyl of R^{d}, are each optionally substituted with 1 to 3 groups selected from ═O and C₁-C₆ alkyl optionally substituted with 1 to 3 groups selected from halogen and —OR^{s};
the phenyl and 5- to 7-membered heteroaryl of R^{d} and the 5- to 7-membered heteroaryl of the C₁-C₁₀ alkyl and C₂-C₁₀ alkenyl of R^{d} are each optionally substituted with 1 to 3 groups selected from C₁-C₆ alkyl optionally substituted with 1 to 3 groups selected from halogen and —OR^{s};
R^{p}, R^{q}, and R^{r}, for each occurrence, are independently selected from hydrogen, C₃-C₁₀ cycloalkyl, and C₁-C₁₀ alkyl, wherein the C₃-C₁₀ cycloalkyl and C₁-C₁₀ alkyl of R^{p}, R^{q}, and R^{r} are each optionally substituted with 1 to 3 groups selected from halogen, —OR^{s} and —C(═O)OR^{s};
R^{s}, for each occurrence, is independently selected from H, C₃-C₁₀ cycloalkyl, 3- to 10-membered heterocyclyl, and C₁-C₁₀ alkyl optionally substituted with 1 to 3 groups selected from halogen, C₃-C₁₀ cycloalkyl, 3- to 10-membered heterocyclyl, and C₁-C₆ alkoxy, wherein
the C₃-C₁₀ cycloalkyl and 3- to 10-membered heterocyclyl of R^{s} and the C₃-C₁₀ cycloalkyl and 3- to 10-membered heterocyclyl of the C₁-C₁₀ alkyl of R^{s} are each optionally substituted with 1 to 3 groups of C₁-C₆ alkyl optionally substituted by halogen, and the C₁-C₆ alkoxy of the C₁-C₁₀ alkyl of R^{s} is optionally substituted with 1 to 3 groups of halogen; all other variables not specifically defined herein are as defined in any one of the preceding embodiments.

In a thirty-eighth embodiment, in a compound, tautomer, a solvate or stereoisomer of the compound or the tautomer, or pharmaceutically acceptable salt of this disclosure, R^{d} is absent or selected from

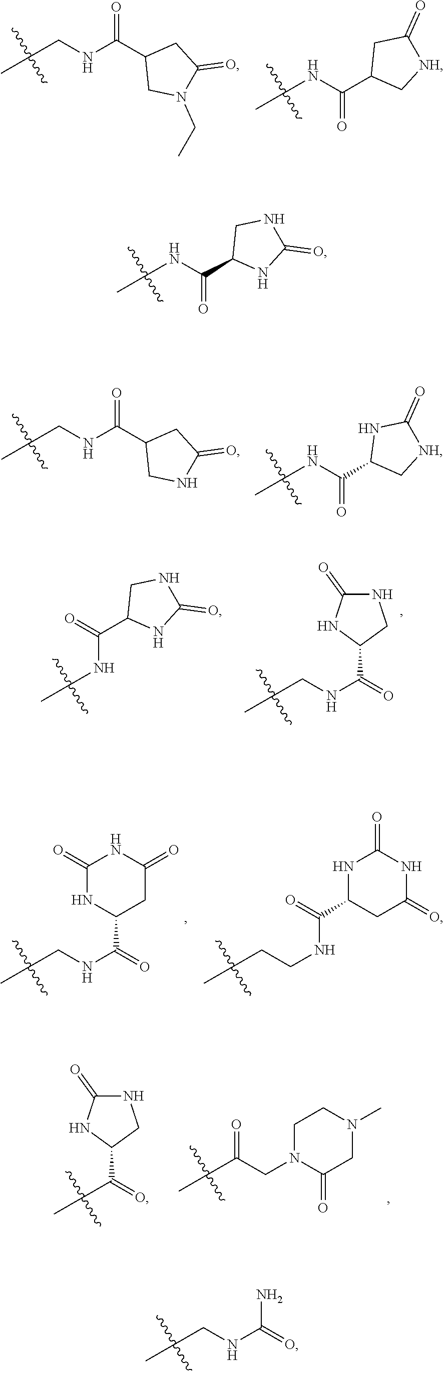

F, methyl, ethyl, propyl, butyl, —(CH₂)₃NCH₃, —CH₂N(CH₃)₂, —CH₂N(CH₃)₃, —(CH₂)₃N(CH₃)₂, —(CH₂)₃NH₂, —(CH₂)₅NH₂, —(CH₂)₄NH₂, —CH₂CH₂NH₂, —CH₂NH₂, —CH₂NHCH₃, —CHCH₃NH₂, —C(CH₃)₂NH₂, —CH₂NH(CH₂)₂OH, —(CH₂)₂NHC(═O)NH₂, —CH₂OCH₂CH₂NH₂, —CH₂CH₂OH, —CH₂OH, —CH₂OCH₃, —CH₂CH₂CH₂OCH₃, —CH₂OCH₂CH₂OCH₃, —CH₂OCH₂CH₂OH, —C(═O)N(CH₃)₂, —C(═O)NHCH₃, —C(═O)NHNH₂, —C(═O)NHOH, —CH₂OH, OH, —O(CH₂)₂N(CH₃)₂, ═O, —C(═O)OCH₃, —C(═O)OH, —C(═O)NH₂, —NH₂, —NHC(═O)CH₃, —NHC(═O)NH₂, —NH(CH₂)₂OH, —NH (CH₂)₃OH, —NH(CH₂)₄OH, —NH(CH₂)₃NH₂, —NH(CH₂)₂NH₂, —NHCH₂CH₂N(CH₃)₂, —NHCH₂CF₃, —NHCH₂CH₂F, —NHCH₂CHF₂, —NHCH₂CH₃, —NHCH₂CH₂OCH₃, —N(CH₃)₂, —NHCH₃, —NHOH, —NHSO₂NH₂, —SO₂NH₂,

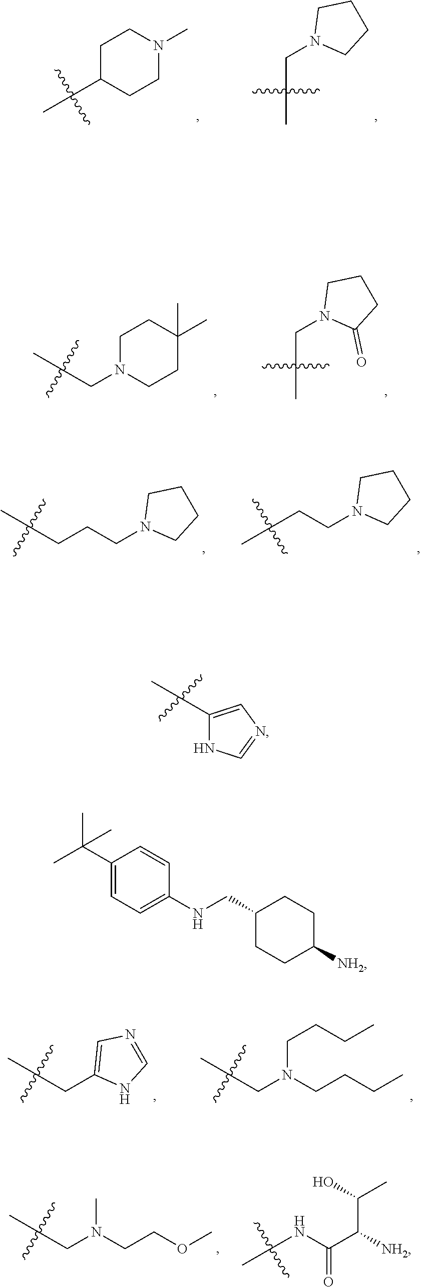

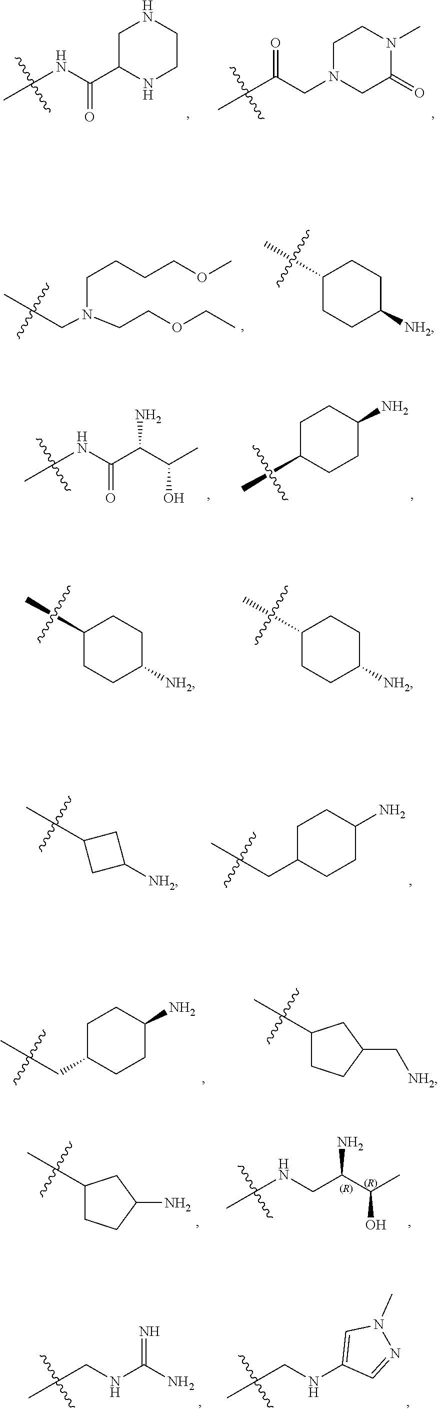

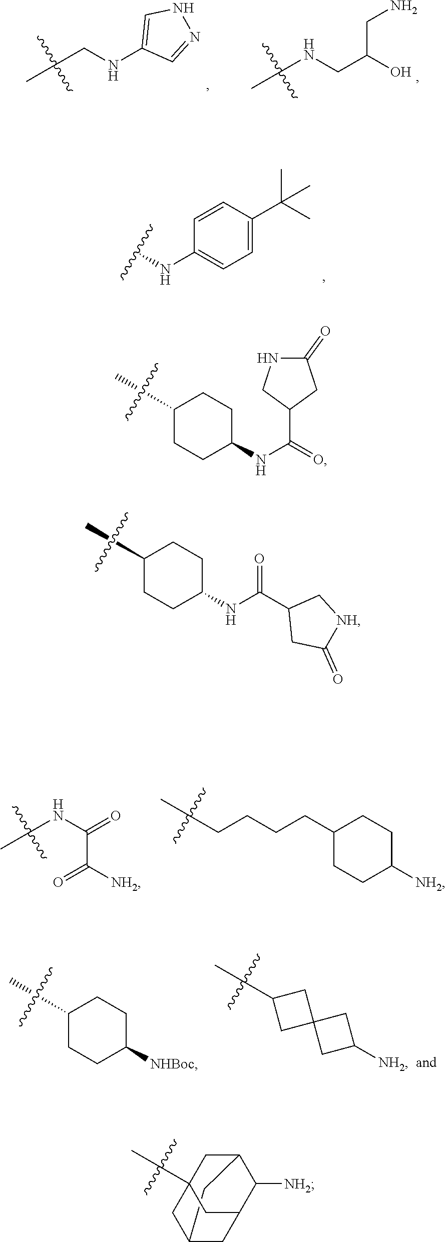

all other variables not specifically defined herein are as defined in any one of the preceding embodiments.

In a thirty-ninth embodiment, in a compound, tautomer, a solvate or stereoisomer of the compound or the tautomer, or pharmaceutically acceptable salt of this disclosure, R^{d} is selected from —C(═O)NR^{p}R^{q}, and C₁-C₆ alkyl, wherein the C₁-C₆ alkyl is optionally substituted with 1 to 3 groups selected from C₁-C₃ alkoxy, —C(═O)NR^{p}R^{q}, and —NR^{p}R^{q}, wherein R^{p} and R^{q} are each selected from H and C₁-C₃ alkyl; all other variables not specifically defined herein are as defined in any one of the preceding embodiments.

In a fortieth embodiment, in a compound, tautomer, a solvate or stereoisomer of the compound or the tautomer, or pharmaceutically acceptable salt of this disclosure, R^{a} and R^{b} join to form a 5- to 6-membered carbocyclic, heterocyclic, aromatic, or heteroaromatic ring optionally substituted with 1 to 4 groups selected from optionally substituted C₁-C₆ alkyl; all other variables not specifically defined herein are as defined in any one of the preceding embodiments.

In a forty-first embodiment, in a compound, tautomer, a solvate or stereoisomer of the compound or the tautomer, or pharmaceutically acceptable salt of this disclosure, R^{a} and R^{b} join to form a structure selected from:

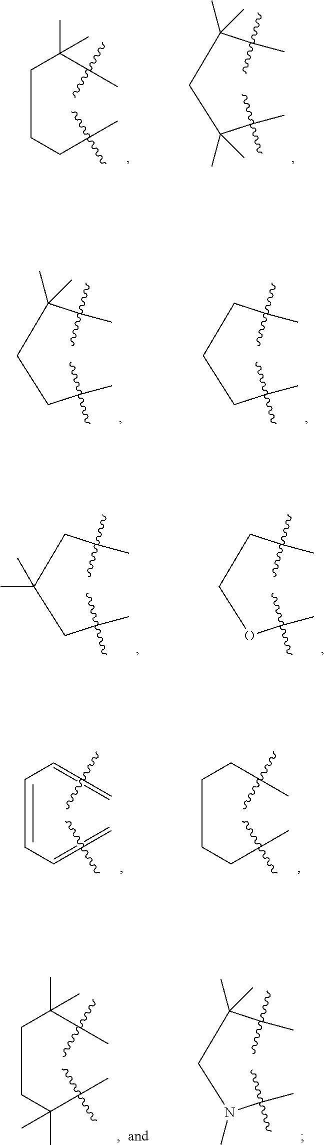

all other variables not specifically defined herein are as defined in any one of the preceding embodiments.

In a forty-second embodiment, in a compound, tautomer, a solvate or stereoisomer of the compound or the tautomer, or pharmaceutically acceptable salt of this disclosure, R^{b} and R^{c} join to form a 5- to 6-membered carbocyclic, heterocyclic, aromatic, or heteroaromatic ring optionally substituted with 1 to 4 groups selected from optionally substituted C₁-C₆ alkyl; all other variables not specifically defined herein are as defined in any one of the preceding embodiments.

In a forty-third embodiment, in a compound, tautomer, a solvate or stereoisomer of the compound or the tautomer, or pharmaceutically acceptable salt of this disclosure, R^{b} and R^{c} join to form a structure selected from:

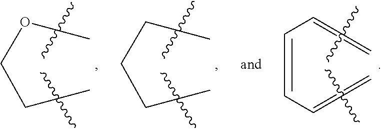 [SMILES: *CCO* ; *CCC* ; *=C/C=C\C=*]

In certain embodiments, the at least one compound of the disclosure is selected from Compounds 1 to 573 depicted in Table 1, a tautomer thereof, a solvate or stereoisomer of the compound depicted in Table 1 or the tautomer, or a pharmaceutically acceptable salt of the foregoing.

In certain embodiments, the at least one compound of the disclosure is selected from Compounds 574 to 661 depicted in Table 1, a tautomer thereof, a solvate or stereoisomer of the compound depicted in Table 1 or the tautomer, or a pharmaceutically acceptable salt of the foregoing.

Certain compounds in this disclosure were prepared and obtained as a mixture of stereoisomers. For example, Compound 124 was prepared and obtained as a mixture of two stereoisomers of the compound in a 1 to 0.3 ratio (described as a “1:0.3 mixture” in the following paragraph), wherein the ratio of the stereoisomers was determined by NMR. In this disclosure, when a compound is denoted with a compound number with an affix, e.g., “A/B” or “A/B/C/D”, it means that stereoisomers, e.g., two or four stereoisomers, denoted as A and B, respectively, or denoted as A, B, C, and D, respectively, of the compound were prepared, isolated, and tested. For example,

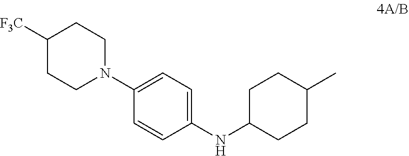 [SMILES: CC1CCC(Nc2ccc(N3CCC(C(F)(F)F)CC3)cc2)CC1]

means that two stereoisomers of compound 4 (4A and 4B) were prepared and isolated (see the paragraph in Example 1) and tested (see the paragraphs in Example 2).

TABLE 1
Compounds 1 to 573 and Compounds 574-661

| | |
|---|---|
| 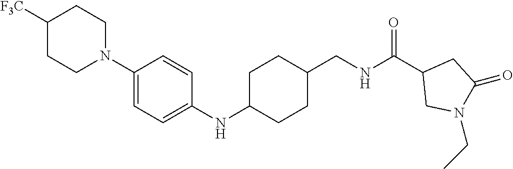 [SMILES: CCN1CC(C(=O)NCC2CCC(Nc3ccc(N4CCC(C(F)(F)F)CC4)cc3)CC2)CC1=O] | 1 |
| 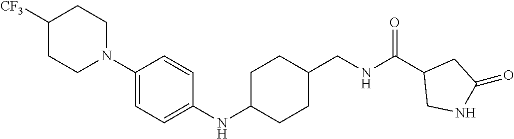 [SMILES: O=C1CC(C(=O)NCC2CCC(Nc3ccc(N4CCC(C(F)(F)F)CC4)cc3)CC2)CN1] | 2 |
| 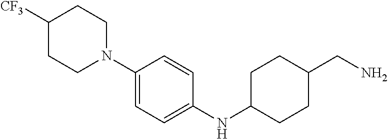 [SMILES: NCC1CCC(Nc2ccc(N3CCC(C(F)(F)F)CC3)cc2)CC1] | 3 |
| 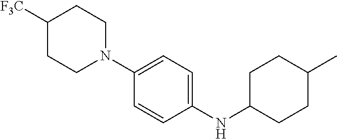 [SMILES: CC1CCC(Nc2ccc(N3CCC(C(F)(F)F)CC3)cc2)CC1] | 4A/B |
| 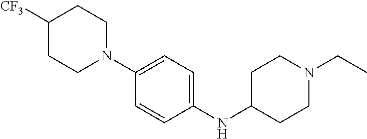 [SMILES: CCN1CCC(Nc2ccc(N3CCC(C(F)(F)F)CC3)cc2)CC1] | 5 |
| 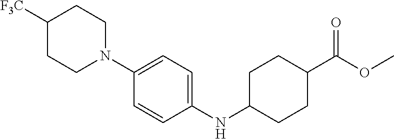 [SMILES: COC(=O)C1CCC(Nc2ccc(N3CCC(C(F)(F)F)CC3)cc2)CC1] | 6A/B |
| 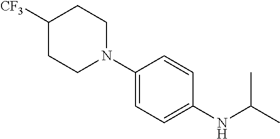 [SMILES: CC(C)Nc1ccc(N2CCC(C(F)(F)F)CC2)cc1] | 7 |
| 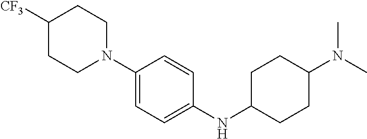 [SMILES: CN(C)C1CCC(Nc2ccc(N3CCC(C(F)(F)F)CC3)cc2)CC1] | 8 |
| 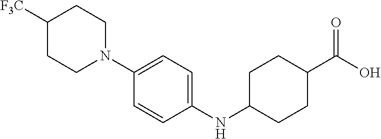 [SMILES: O=C(O)C1CCC(Nc2ccc(N3CCC(C(F)(F)F)CC3)cc2)CC1] | 9 |
| 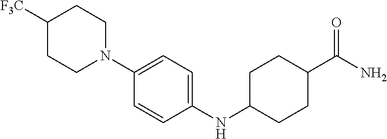 [SMILES: NC(=O)C1CCC(Nc2ccc(N3CCC(C(F)(F)F)CC3)cc2)CC1] | 10 |
| 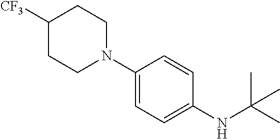 [SMILES: CC(C)(C)Nc1ccc(N2CCC(C(F)(F)F)CC2)cc1] | 11 |
| 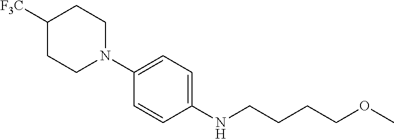 [SMILES: COCCCCNc1ccc(N2CCC(C(F)(F)F)CC2)cc1] | 12 |
| 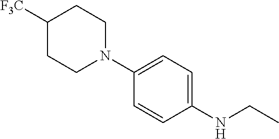 [SMILES: CCNc1ccc(N2CCC(C(F)(F)F)CC2)cc1] | 13 |
| 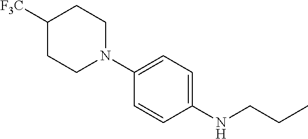 [SMILES: CCCNc1ccc(N2CCC(C(F)(F)F)CC2)cc1] | 14 |
| 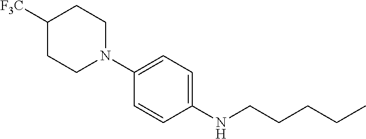 [SMILES: CCCCCNc1ccc(N2CCC(C(F)(F)F)CC2)cc1] | 15 |
| 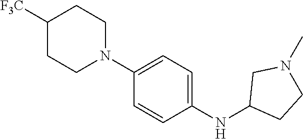 [SMILES: CN1CCC(Nc2ccc(N3CCC(C(F)(F)F)CC3)cc2)C1] | 16 |
| 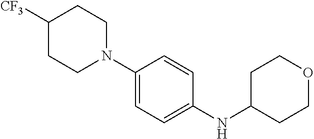 [SMILES: FC(F)(F)C1CCN(c2ccc(NC3CCOCC3)cc2)CC1] | 17 |
| 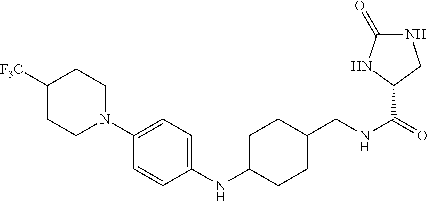 [SMILES: O=C1NC[C@H](C(=O)NCC2CCC(Nc3ccc(N4CCC(C(F)(F)F)CC4)cc3)CC2)N1] | 18 |
| 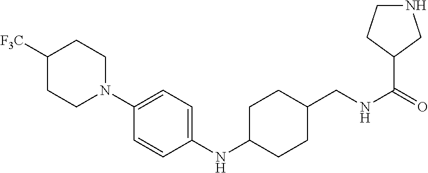 [SMILES: O=C(NCC1CCC(Nc2ccc(N3CCC(C(F)(F)F)CC3)cc2)CC1)C1CCNC1] | 19 |
| 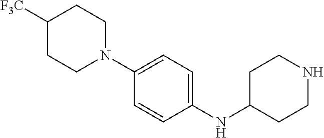 [SMILES: FC(F)(F)C1CCN(c2ccc(NC3CCNCC3)cc2)CC1] | 20 |
| 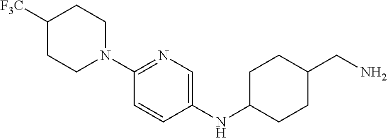 [SMILES: NCC1CCC(Nc2ccc(N3CCC(C(F)(F)F)CC3)nc2)CC1] | 21 |
| 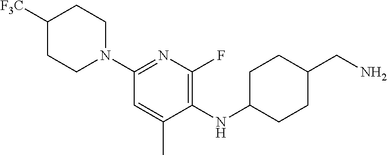 [SMILES: Cc1cc(N2CCC(C(F)(F)F)CC2)nc(F)c1NC1CCC(CN)CC1] | 22 |
| 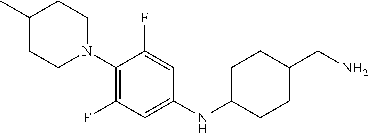 [SMILES: CC1CCN(c2c(F)cc(NC3CCC(CN)CC3)cc2F)CC1] | 23A/B |
| 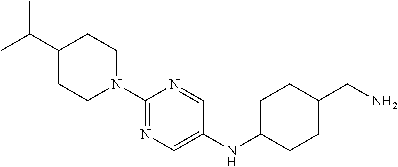 [SMILES: CC(C)C1CCN(c2ncc(NC3CCC(CN)CC3)cn2)CC1] | 24A/B |
| 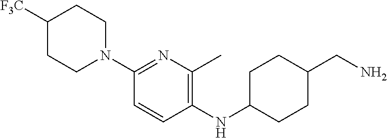 [SMILES: Cc1nc(N2CCC(C(F)(F)F)CC2)ccc1NC1CCC(CN)CC1] | 25 |
| 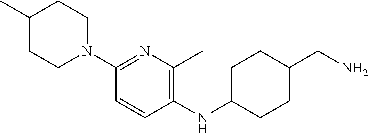 [SMILES: Cc1nc(N2CCC(C)CC2)ccc1NC1CCC(CN)CC1] | 26 |
| 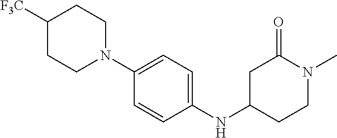 [SMILES: CN1CCC(Nc2ccc(N3CCC(C(F)(F)F)CC3)cc2)CC1=O] | 27 |
| 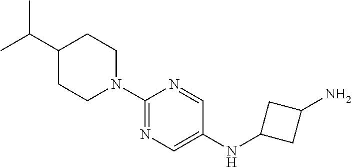 [SMILES: CC(C)C1CCN(c2ncc(NC3CC(N)C3)cn2)CC1] | 28 |
| 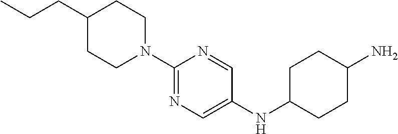 [SMILES: CCCC1CCN(c2ncc(NC3CCC(N)CC3)cn2)CC1] | 29 |
| 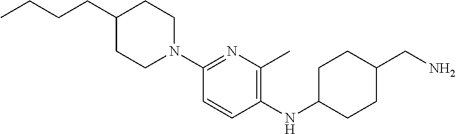 [SMILES: CCCCC1CCN(c2ccc(NC3CCC(CN)CC3)c(C)n2)CC1] | 30 |
| 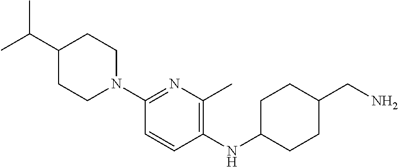 [SMILES: Cc1nc(N2CCC(C(C)C)CC2)ccc1NC1CCC(CN)CC1] | 31 |
| 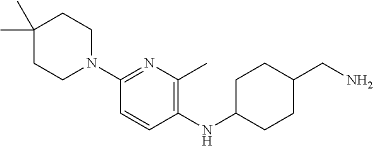 [SMILES: Cc1nc(N2CCC(C)(C)CC2)ccc1NC1CCC(CN)CC1] | 32A/B |
| 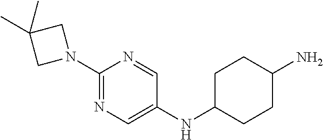 [SMILES: CC1(C)CN(c2ncc(NC3CCC(N)CC3)cn2)C1] | 33A/B |
| 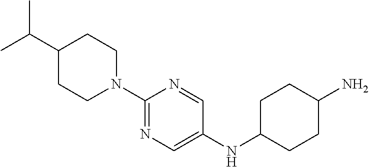 [SMILES: CC(C)C1CCN(c2ncc(NC3CCC(N)CC3)cn2)CC1] | 34A/B |
| 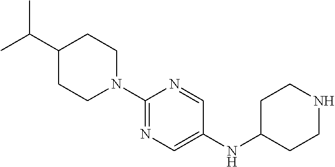 [SMILES: CC(C)C1CCN(c2ncc(NC3CCNCC3)cn2)CC1] | 35 |
| 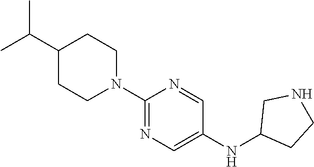 [SMILES: CC(C)C1CCN(c2ncc(NC3CCNC3)cn2)CC1] | 36 |
| 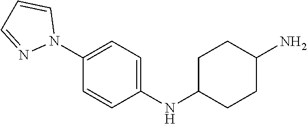 [SMILES: NC1CCC(Nc2ccc(-n3cccn3)cc2)CC1] | 37A/B |
| 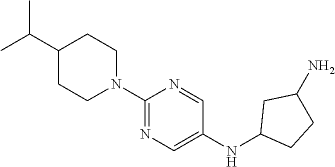 [SMILES: CC(C)C1CCN(c2ncc(NC3CCC(N)C3)cn2)CC1] | 38 |
| 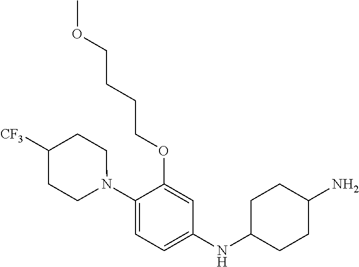 [SMILES: COCCCCOc1cc(NC2CCC(N)CC2)ccc1N1CCC(C(F)(F)F)CC1] | 39 |
| 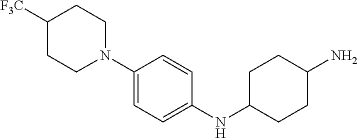 [SMILES: NC1CCC(Nc2ccc(N3CCC(C(F)(F)F)CC3)cc2)CC1] | 40 |
| 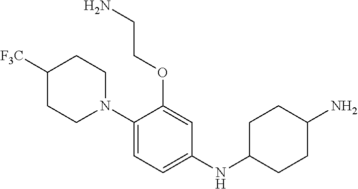 [SMILES: NCCOc1cc(NC2CCC(N)CC2)ccc1N1CCC(C(F)(F)F)CC1] | 41A/B |
| 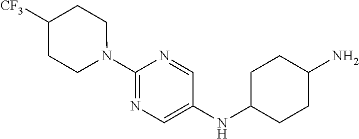 [SMILES: NC1CCC(Nc2cnc(N3CCC(C(F)(F)F)CC3)nc2)CC1] | 42 |
| 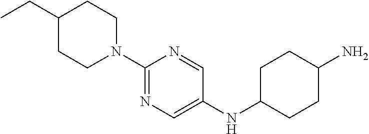 [SMILES: CCC1CCN(c2ncc(NC3CCC(N)CC3)cn2)CC1] | 43 |
| 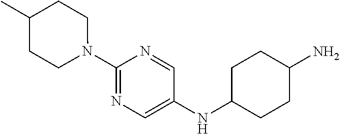 [SMILES: CC1CCN(c2ncc(NC3CCC(N)CC3)cn2)CC1] | 44 |
| 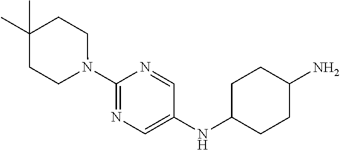 [SMILES: CC1(C)CCN(c2ncc(NC3CCC(N)CC3)cn2)CC1] | 45 |
| 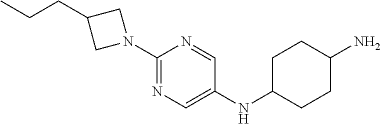 [SMILES: CCCC1CN(c2ncc(NC3CCC(N)CC3)cn2)C1] | 46 |
| 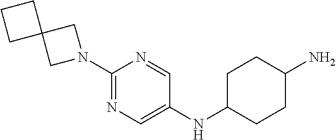 [SMILES: NC1CCC(Nc2cnc(N3CC4(CCC4)C3)nc2)CC1] | 47 |
| 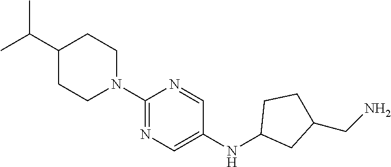 [SMILES: CC(C)C1CCN(c2ncc(NC3CCC(CN)C3)cn2)CC1] | 48 |
| 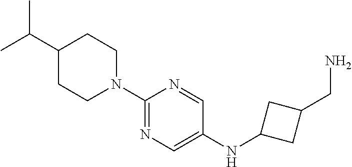 [SMILES: CC(C)C1CCN(c2ncc(NC3CC(CN)C3)cn2)CC1] | 49 |
| 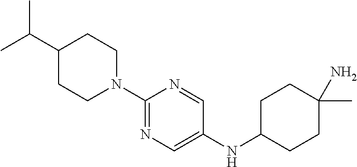 [SMILES: CC(C)C1CCN(c2ncc(NC3CCC(C)(N)CC3)cn2)CC1] | 50 |
| 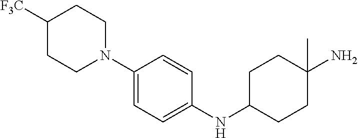 [SMILES: CC1(N)CCC(Nc2ccc(N3CCC(C(F)(F)F)CC3)cc2)CC1] | 51 |
| 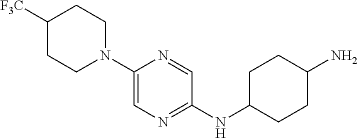 [SMILES: NC1CCC(Nc2cnc(N3CCC(C(F)(F)F)CC3)cn2)CC1] | 52 |
| 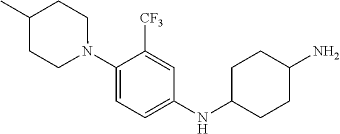 [SMILES: CC1CCN(c2ccc(NC3CCC(N)CC3)cc2C(F)(F)F)CC1] | 53A/B |
| 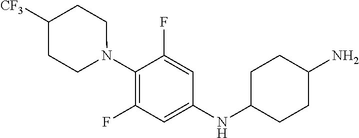 [SMILES: NC1CCC(Nc2cc(F)c(N3CCC(C(F)(F)F)CC3)c(F)c2)CC1] | 54A/B |
| 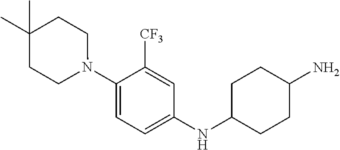 [SMILES: CC1(C)CCN(c2ccc(NC3CCC(N)CC3)cc2C(F)(F)F)CC1] | 55 |
| 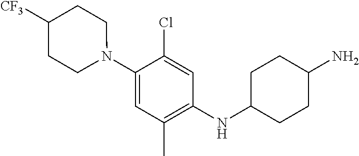 [SMILES: Cc1cc(N2CCC(C(F)(F)F)CC2)c(Cl)cc1NC1CCC(N)CC1] | 56 |
| 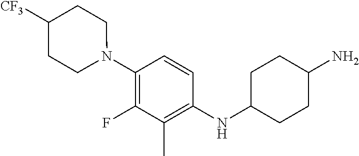 [SMILES: Cc1c(NC2CCC(N)CC2)ccc(N2CCC(C(F)(F)F)CC2)c1F] | 57 |
| 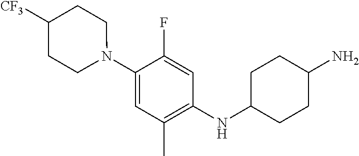 [SMILES: Cc1cc(N2CCC(C(F)(F)F)CC2)c(F)cc1NC1CCC(N)CC1] | 58 |
| 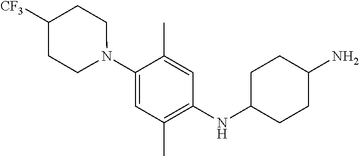 [SMILES: Cc1cc(N2CCC(C(F)(F)F)CC2)c(C)cc1NC1CCC(N)CC1] | 59 |
| 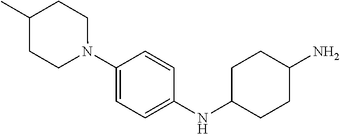 [SMILES: CC1CCN(c2ccc(NC3CCC(N)CC3)cc2)CC1] | 60 |
| 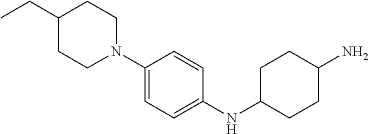 [SMILES: CCC1CCN(c2ccc(NC3CCC(N)CC3)cc2)CC1] | 61 |
| 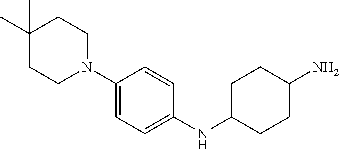 [SMILES: CC1(C)CCN(c2ccc(NC3CCC(N)CC3)cc2)CC1] | 62 |
| 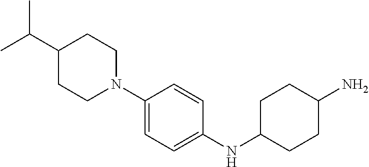 [SMILES: CC(C)C1CCN(c2ccc(NC3CCC(N)CC3)cc2)CC1] | 63 |
| 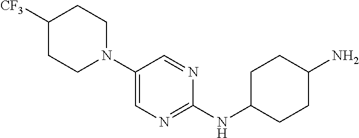 [SMILES: NC1CCC(Nc2ncc(N3CCC(C(F)(F)F)CC3)cn2)CC1] | 64A/B |
| 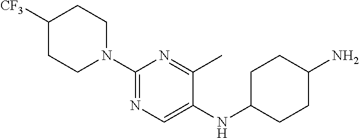 [SMILES: Cc1nc(N2CCC(C(F)(F)F)CC2)ncc1NC1CCC(N)CC1] | 65A/B |
| 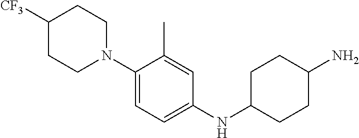 [SMILES: Cc1cc(NC2CCC(N)CC2)ccc1N1CCC(C(F)(F)F)CC1] | 66 |
| 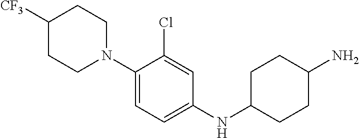 [SMILES: NC1CCC(Nc2ccc(N3CCC(C(F)(F)F)CC3)c(Cl)c2)CC1] | 67 |
| 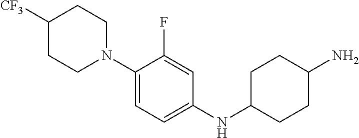 [SMILES: NC1CCC(Nc2ccc(N3CCC(C(F)(F)F)CC3)c(F)c2)CC1] | 68 |
| 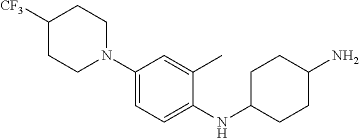 [SMILES: Cc1cc(N2CCC(C(F)(F)F)CC2)ccc1NC1CCC(N)CC1] | 69 |
| 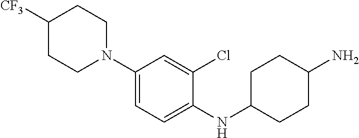 [SMILES: NC1CCC(Nc2ccc(N3CCC(C(F)(F)F)CC3)cc2Cl)CC1] | 70 |
| 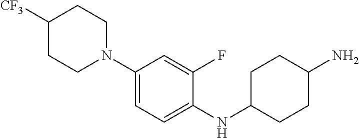 [SMILES: NC1CCC(Nc2ccc(N3CCC(C(F)(F)F)CC3)cc2F)CC1] | 71 |
| 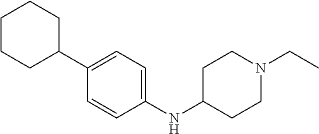 [SMILES: CCN1CCC(Nc2ccc(C3CCCCC3)cc2)CC1] | 72 |
| 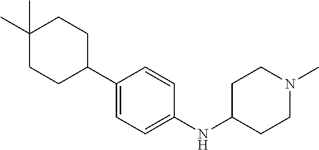 | 73 |
| 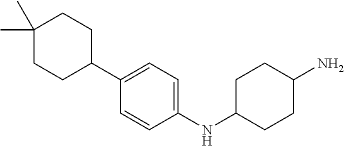 [SMILES: CC1(C)CCC(c2ccc(NC3CCC(N)CC3)cc2)CC1] | 74 |
| 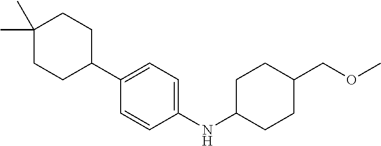 | 75 |
| 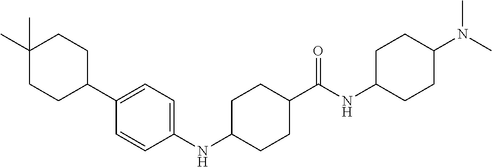 | 76A/B |
| 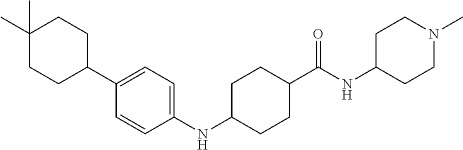 | 77 |
| 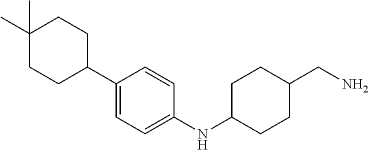 [SMILES: CC1(C)CCC(c2ccc(NC3CCC(CN)CC3)cc2)CC1] | 78 |
| 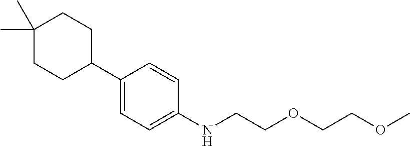 | 79 |
| 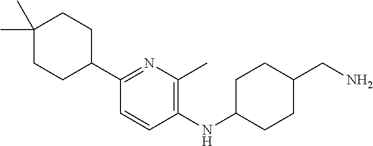 [SMILES: Cc1nc(C2CCC(C)(C)CC2)ccc1NC1CCC(CN)CC1] | 80 |
| 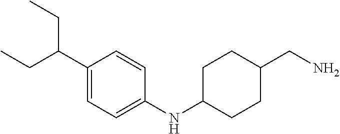 [SMILES: CCC(CC)c1ccc(NC2CCC(CN)CC2)cc1] | 81A/B |
| 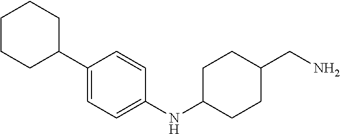 [SMILES: NCC1CCC(Nc2ccc(C3CCCCC3)cc2)CC1] | 82A/B |
| 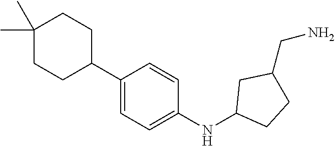 [SMILES: CC1(C)CCC(c2ccc(NC3CCC(CN)C3)cc2)CC1] | 83 |
| 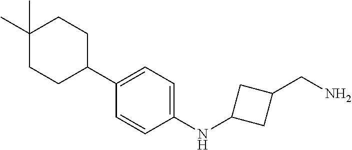 [SMILES: CC1(C)CCC(c2ccc(NC3CC(CN)C3)cc2)CC1] | 84 |
| 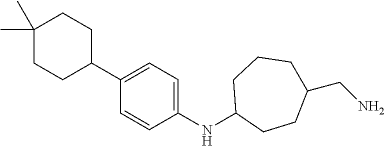 [SMILES: CC1(C)CCC(c2ccc(NC3CCCC(CN)CC3)cc2)CC1] | 85 |
| 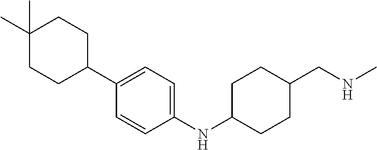 | 86 |
| 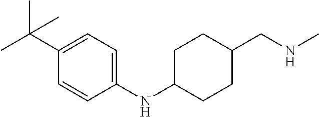 [SMILES: CNCC1CCC(Nc2ccc(C(C)(C)C)cc2)CC1] | 87 |
| 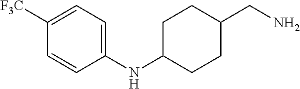 [SMILES: NCC1CCC(Nc2ccc(C(F)(F)F)cc2)CC1] | 88 |
| 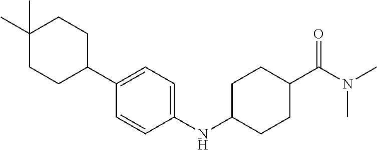 | 89 |
| 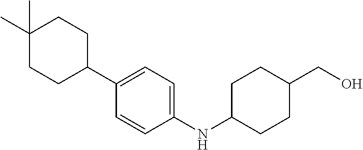 [SMILES: CC1(C)CCC(c2ccc(NC3CCC(CO)CC3)cc2)CC1] | 90 |
| 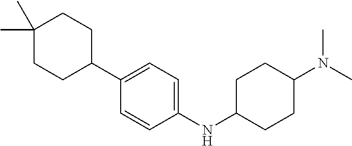 | 91A/B |
| 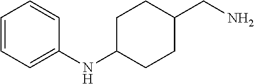 [SMILES: NCC1CCC(Nc2ccccc2)CC1] | 92 |
| 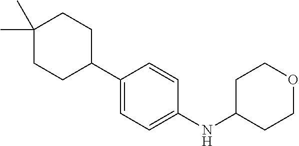 [SMILES: CC1(C)CCC(c2ccc(NC3CCOCC3)cc2)CC1] | 93 |
| 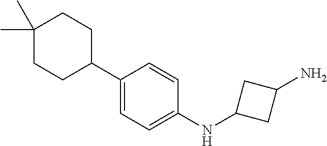 [SMILES: CC1(C)CCC(c2ccc(NC3CC(N)C3)cc2)CC1] | 94 |
| 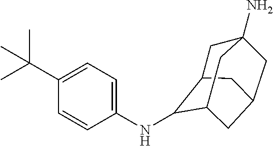 [SMILES: CC(C)(C)c1ccc(NC2C3CC4CC2CC(N)(C4)C3)cc1] | 95 |
| 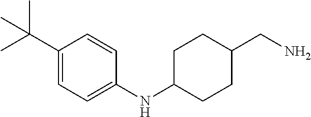 [SMILES: CC(C)(C)c1ccc(NC2CCC(CN)CC2)cc1] | 96 |
| 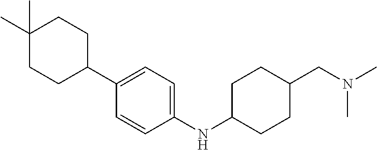 | 97A/B |
| 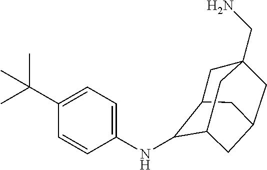 [SMILES: CC(C)(C)c1ccc(NC2C3CC4CC2CC(CN)(C4)C3)cc1] | 98 |
| 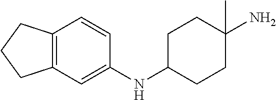 [SMILES: CC1(N)CCC(Nc2ccc3c(c2)CCC3)CC1] | 99 |
| 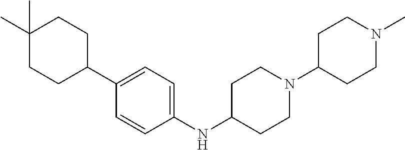 | 100 |
| 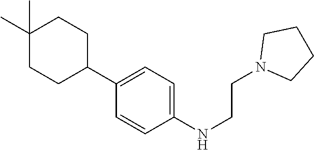 [SMILES: CC1(C)CCC(c2ccc(NCCN3CCCC3)cc2)CC1] | 101 |
| 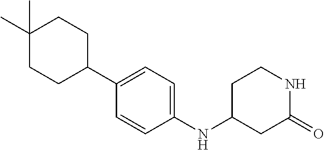 [SMILES: CC1(C)CCC(c2ccc(NC3CCNC(=O)C3)cc2)CC1] | 102 |
| 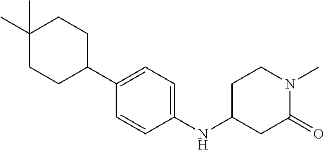 | 103 |
| 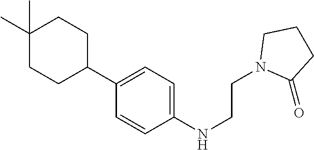 [SMILES: CC1(C)CCC(c2ccc(NCCN3CCCC3=O)cc2)CC1] | 104 |
| 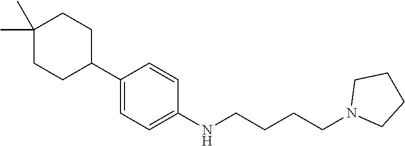 [SMILES: CC1(C)CCC(c2ccc(NCCCCN3CCCC3)cc2)CC1] | 105 |
| 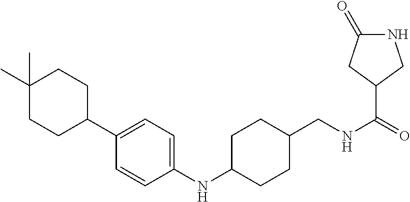 [SMILES: CC1(C)CCC(c2ccc(NC3CCC(CNC(=O)C4CNC(=O)C4)CC3)cc2)CC1] | 106A/B |
| 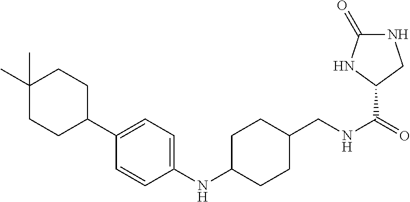 [SMILES: CC1(C)CCC(c2ccc(NC3CCC(CNC(=O)[C@H]4CNC(=O)N4)CC3)cc2)CC1] | 107A/B |
| 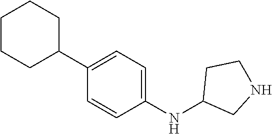 [SMILES: c1cc(C2CCCCC2)ccc1NC1CCNC1] | 108 |
| 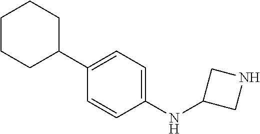 [SMILES: c1cc(C2CCCCC2)ccc1NC1CNC1] | 109 |
| 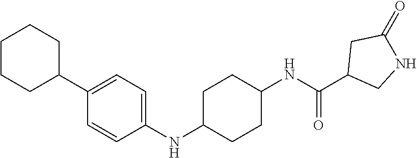 [SMILES: O=C1CC(C(=O)NC2CCC(Nc3ccc(C4CCCCC4)cc3)CC2)CN1] | 110 |
| 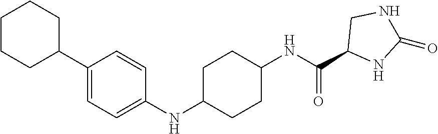 [SMILES: O=C1NC[C@H](C(=O)NC2CCC(Nc3ccc(C4CCCCC4)cc3)CC2)N1] | 111 |
| 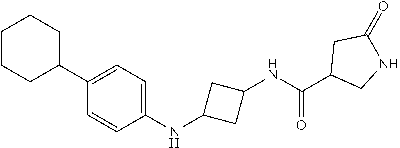 [SMILES: O=C1CC(C(=O)NC2CC(Nc3ccc(C4CCCCC4)cc3)C2)CN1] | 112 |
| 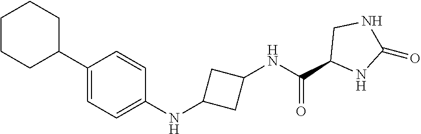 [SMILES: O=C1NC[C@H](C(=O)NC2CC(Nc3ccc(C4CCCCC4)cc3)C2)N1] | 113 |
| 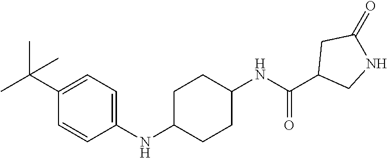 [SMILES: CC(C)(C)c1ccc(NC2CCC(NC(=O)C3CNC(=O)C3)CC2)cc1] | 114A/B |
| 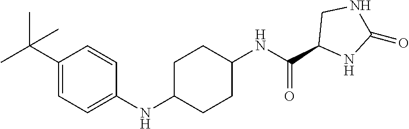 [SMILES: CC(C)(C)c1ccc(NC2CCC(NC(=O)[C@H]3CNC(=O)N3)CC2)cc1] | 115A/B |
| 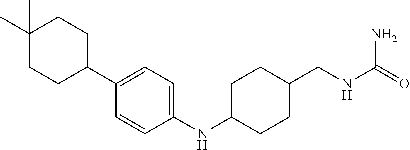 [SMILES: CC1(C)CCC(c2ccc(NC3CCC(CNC(N)=O)CC3)cc2)CC1] | 116 |
| 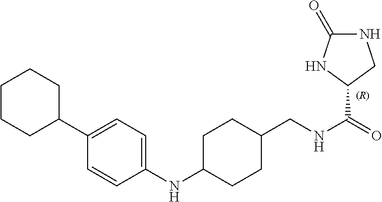 | 117A/B |
| 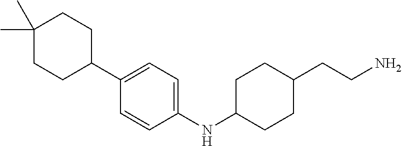 [SMILES: CC1(C)CCC(c2ccc(NC3CCC(CCN)CC3)cc2)CC1] | 118 |
| 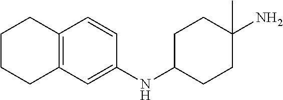 [SMILES: CC1(N)CCC(Nc2ccc3c(c2)CCCC3)CC1] | 119 |
| 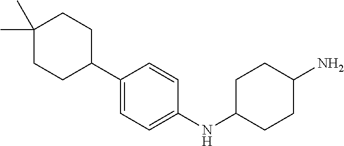 [SMILES: CC1(C)CCC(c2ccc(NC3CCC(N)CC3)cc2)CC1] | 120A/B |
| 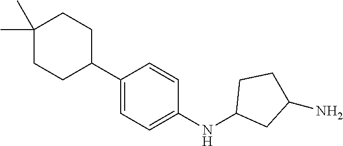 [SMILES: CC1(C)CCC(c2ccc(NC3CCC(N)C3)cc2)CC1] | 121A/B |
| 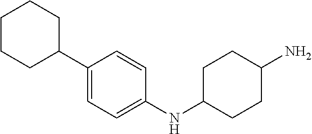 [SMILES: NC1CCC(Nc2ccc(C3CCCCC3)cc2)CC1] | 122 |
| 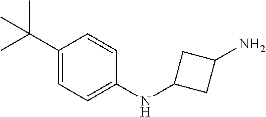 [SMILES: CC(C)(C)c1ccc(NC2CC(N)C2)cc1] | 123 |
| 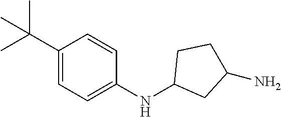 [SMILES: CC(C)(C)c1ccc(NC2CCC(N)C2)cc1] | 124 |
| 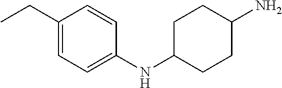 [SMILES: CCc1ccc(NC2CCC(N)CC2)cc1] | 125 |
| 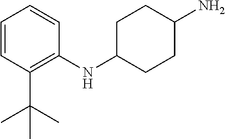 [SMILES: CC(C)(C)c1ccccc1NC1CCC(N)CC1] | 126 |
| 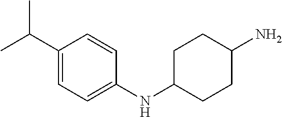 [SMILES: CC(C)c1ccc(NC2CCC(N)CC2)cc1] | 127 |
| 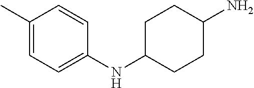 | 128 |
| 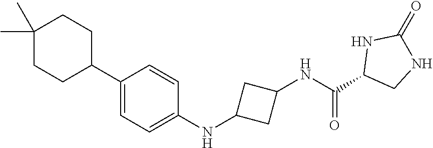 [SMILES: CC1(C)CCC(c2ccc(NC3CC(NC(=O)[C@H]4CNC(=O)N4)C3)cc2)CC1] | 129 |
| 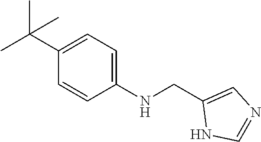 [SMILES: CC(C)(C)c1ccc(NCc2cnc[nH]2)cc1] | 130 |
| 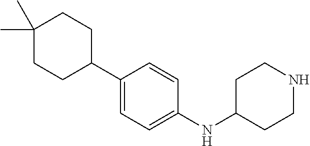 [SMILES: CC1(C)CCC(c2ccc(NC3CCNCC3)cc2)CC1] | 131 |
| 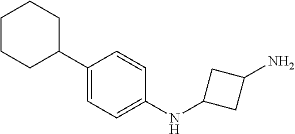 [SMILES: NC1CC(Nc2ccc(C3CCCCC3)cc2)C1] | 132 |
| 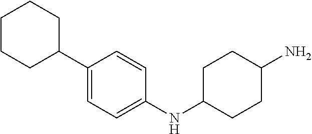 [SMILES: NC1CCC(Nc2ccc(C3CCCCC3)cc2)CC1] | 133 |
| 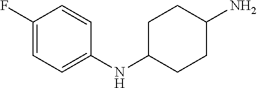 [SMILES: NC1CCC(Nc2ccc(F)cc2)CC1] | 134 |
| 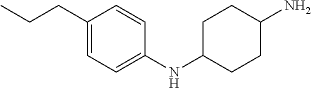 [SMILES: CCCc1ccc(NC2CCC(N)CC2)cc1] | 135A/B |
| 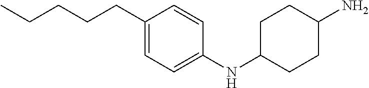 [SMILES: CCCCCc1ccc(NC2CCC(N)CC2)cc1] | 136 |
| 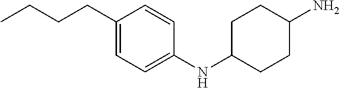 [SMILES: CCCCc1ccc(NC2CCC(N)CC2)cc1] | 137 |
| 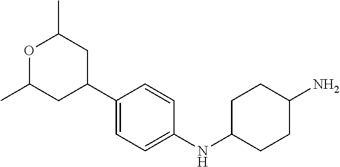 [SMILES: CC1CC(c2ccc(NC3CCC(N)CC3)cc2)CC(C)O1] | 138 |
| 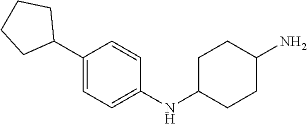 [SMILES: NC1CCC(Nc2ccc(C3CCCC3)cc2)CC1] | 139A/B |
| 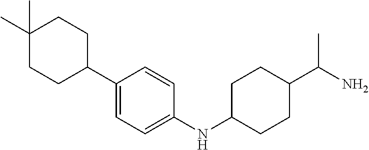 [SMILES: CC(N)C1CCC(Nc2ccc(C3CCC(C)(C)CC3)cc2)CC1] | 140 |
| 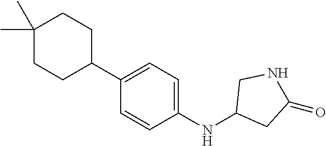 [SMILES: CC1(C)CCC(c2ccc(NC3CNC(=O)C3)cc2)CC1] | 141 |
| 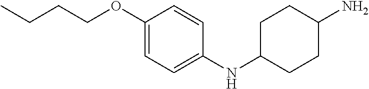 [SMILES: CCCCOc1ccc(NC2CCC(N)CC2)cc1] | 142A/B |
| 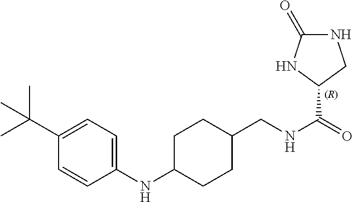 | 143A/B |
| 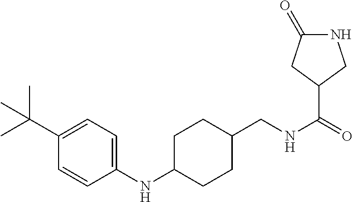 [SMILES: CC(C)(C)c1ccc(NC2CCC(CNC(=O)C3CNC(=O)C3)CC2)cc1] | 144A/B |
| 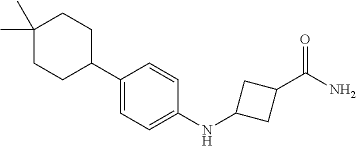 [SMILES: CC1(C)CCC(c2ccc(NC3CC(C(N)=O)C3)cc2)CC1] | 145 |
| 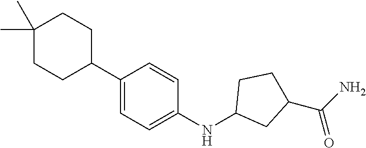 [SMILES: CC1(C)CCC(c2ccc(NC3CCC(C(N)=O)C3)cc2)CC1] | 146A/B |
| 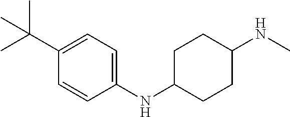 [SMILES: CNC1CCC(Nc2ccc(C(C)(C)C)cc2)CC1] | 147 |
| 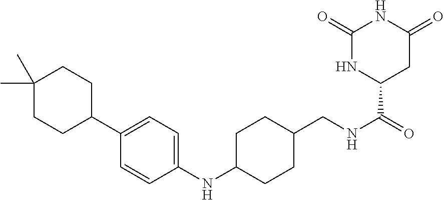 [SMILES: CC1(C)CCC(c2ccc(NC3CCC(CNC(=O)[C@H]4CC(=O)NC(=O)N4)CC3)cc2)CC1] | 148A/B |
| 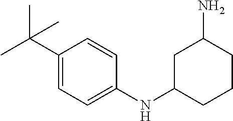 [SMILES: CC(C)(C)c1ccc(NC2CCCC(N)C2)cc1] | 149A/B |
| 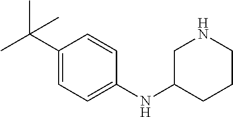 [SMILES: CC(C)(C)c1ccc(NC2CCCNC2)cc1] | 150 |
| 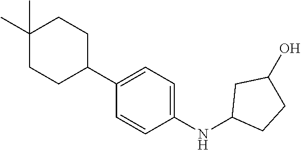 [SMILES: CC1(C)CCC(c2ccc(NC3CCC(O)C3)cc2)CC1] | 151 |
| 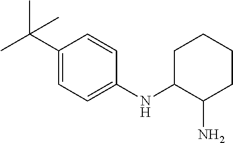 [SMILES: CC(C)(C)c1ccc(NC2CCCCC2N)cc1] | 152 |
| 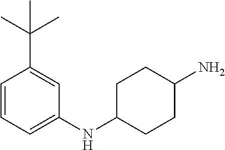 [SMILES: CC(C)(C)c1cccc(NC2CCC(N)CC2)c1] | 153A/B |
| 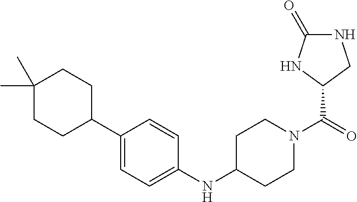 [SMILES: CC1(C)CCC(c2ccc(NC3CCN(C(=O)[C@H]4CNC(=O)N4)CC3)cc2)CC1] | 154 |
| 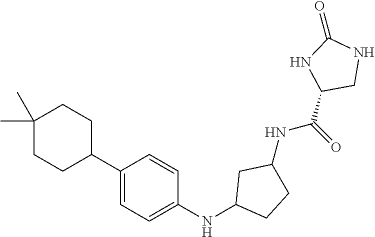 [SMILES: CC1(C)CCC(c2ccc(NC3CCC(NC(=O)[C@H]4CNC(=O)N4)C3)cc2)CC1] | 155 |
| 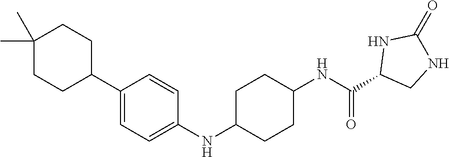 [SMILES: CC1(C)CCC(c2ccc(NC3CCC(NC(=O)[C@H]4CNC(=O)N4)CC3)cc2)CC1] | 156A/B |
| 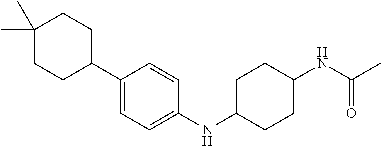 [SMILES: CC(=O)NC1CCC(Nc2ccc(C3CCC(C)(C)CC3)cc2)CC1] | 157A/B |
| 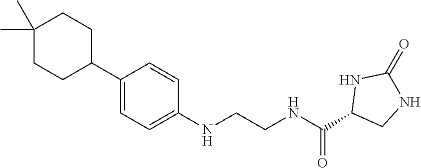 [SMILES: CC1(C)CCC(c2ccc(NCCNC(=O)[C@H]3CNC(=O)N3)cc2)CC1] | 158 |
| 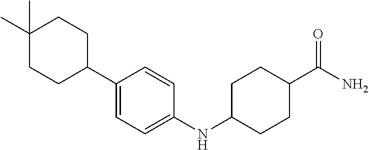 [SMILES: CC1(C)CCC(c2ccc(NC3CCC(C(N)=O)CC3)cc2)CC1] | 159A/B |
| 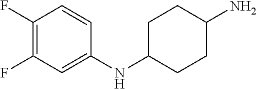 [SMILES: NC1CCC(Nc2ccc(F)c(F)c2)CC1] | 160 |
| 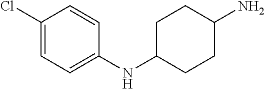 [SMILES: NC1CCC(Nc2ccc(Cl)cc2)CC1] | 161 |
| 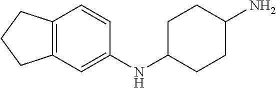 [SMILES: NC1CCC(Nc2ccc3c(c2)CCC3)CC1] | 162 |
| 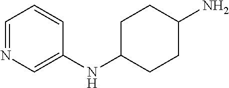 [SMILES: NC1CCC(Nc2cccnc2)CC1] | 163 |
| 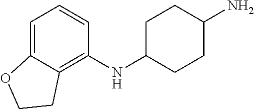 [SMILES: NC1CCC(Nc2cccc3c2CCO3)CC1] | 164 |
| 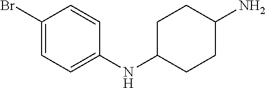 [SMILES: NC1CCC(Nc2ccc(Br)cc2)CC1] | 165 |
| 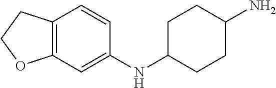 [SMILES: NC1CCC(Nc2ccc3c(c2)OCC3)CC1] | 166 |
| 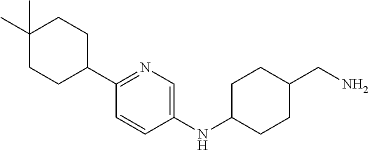 [SMILES: CC1(C)CCC(c2ccc(NC3CCC(CN)CC3)cn2)CC1] | 167 |
| 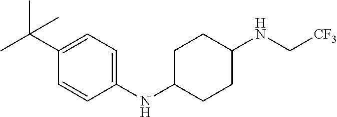 [SMILES: CC(C)(C)c1ccc(NC2CCC(NCC(F)(F)F)CC2)cc1] | 168A/B |
| 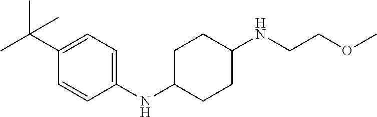 [SMILES: COCCNC1CCC(Nc2ccc(C(C)(C)C)cc2)CC1] | 169 |
| 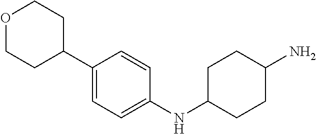 [SMILES: NC1CCC(Nc2ccc(C3CCOCC3)cc2)CC1] | 170 |
| 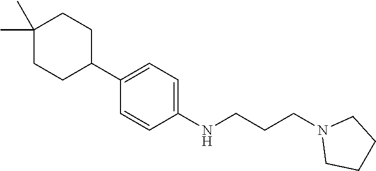 [SMILES: CC1(C)CCC(c2ccc(NCCCN3CCCC3)cc2)CC1] | 171 |
| 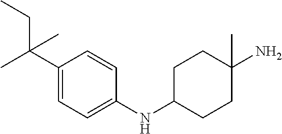 [SMILES: CCC(C)(C)c1ccc(NC2CCC(C)(N)CC2)cc1] | 172 |
| 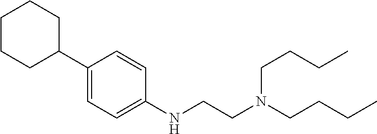 [SMILES: CCCCN(CCCC)CCNc1ccc(C2CCCCC2)cc1] | 173 |
| 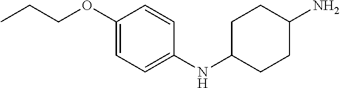 [SMILES: CCCOc1ccc(NC2CCC(N)CC2)cc1] | 174 |
| 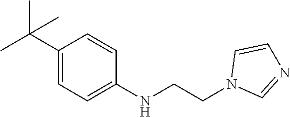 [SMILES: CC(C)(C)c1ccc(NCCn2ccnc2)cc1] | 175 |
| 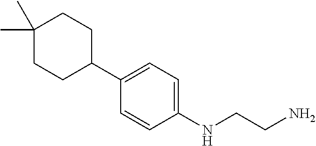 [SMILES: CC1(C)CCC(c2ccc(NCCN)cc2)CC1] | 176 |
| 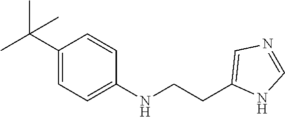 [SMILES: CC(C)(C)c1ccc(NCCc2cnc[nH]2)cc1] | 177 |
| 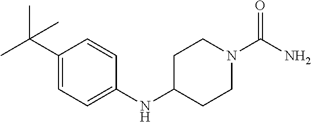 [SMILES: CC(C)(C)c1ccc(NC2CCN(C(N)=O)CC2)cc1] | 178 |
| 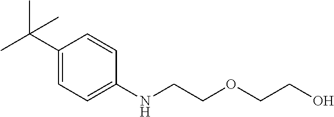 [SMILES: CC(C)(C)c1ccc(NCCOCCO)cc1] | 179 |
| 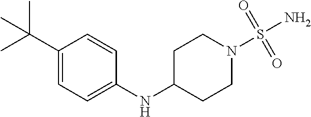 [SMILES: CC(C)(C)c1ccc(NC2CCN(S(N)(=O)=O)CC2)cc1] | 180 |
| 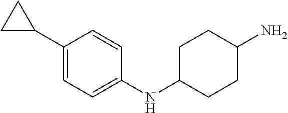 [SMILES: NC1CCC(Nc2ccc(C3CC3)cc2)CC1] | 181 |
| 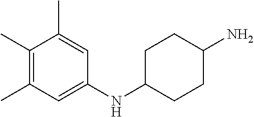 [SMILES: Cc1cc(NC2CCC(N)CC2)cc(C)c1C] | 182A/B |
| 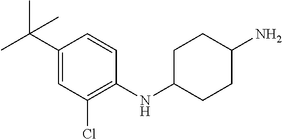 [SMILES: CC(C)(C)c1ccc(NC2CCC(N)CC2)c(Cl)c1] | 183A/B |
| 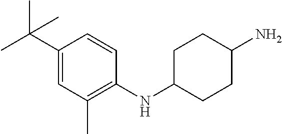 [SMILES: Cc1cc(C(C)(C)C)ccc1NC1CCC(N)CC1] | 184 |
| 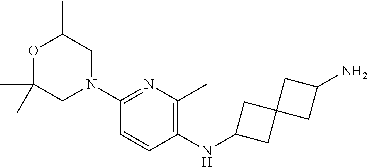 [SMILES: Cc1nc(N2CC(C)OC(C)(C)C2)ccc1NC1CC2(CC(N)C2)C1] | 185 |
| 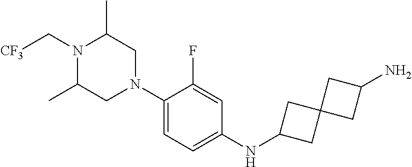 [SMILES: CC1CN(c2ccc(NC3CC4(CC(N)C4)C3)cc2F)CC(C)N1CC(F)(F)F] | 186 |
| 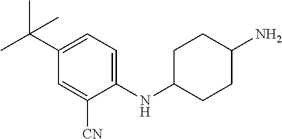 [SMILES: CC(C)(C)c1ccc(NC2CCC(N)CC2)c(C#N)c1] | 187 |
| 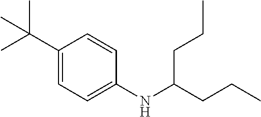 [SMILES: CCCC(CCC)Nc1ccc(C(C)(C)C)cc1] | 188 |
| 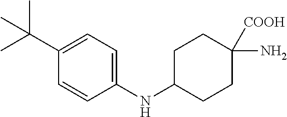 [SMILES: CC(C)(C)c1ccc(NC2CCC(N)(C(=O)O)CC2)cc1] | 189 |
| 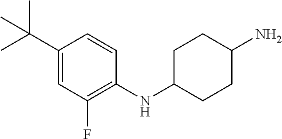 [SMILES: CC(C)(C)c1ccc(NC2CCC(N)CC2)c(F)c1] | 190 |
| 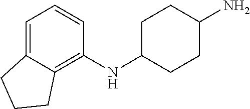 [SMILES: NC1CCC(Nc2cccc3c2CCC3)CC1] | 191A/B |
| 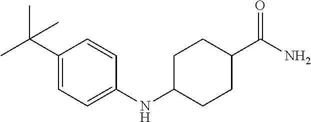 [SMILES: CC(C)(C)c1ccc(NC2CCC(C(N)=O)CC2)cc1] | 192 |
| 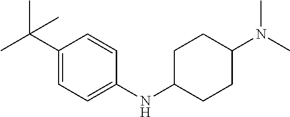 [SMILES: CN(C)C1CCC(Nc2ccc(C(C)(C)C)cc2)CC1] | 193A/B |
| 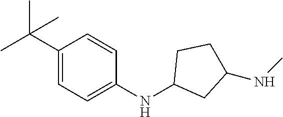 [SMILES: CNC1CCC(Nc2ccc(C(C)(C)C)cc2)C1] | 194 |
| 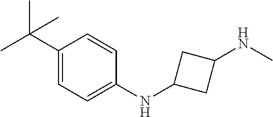 [SMILES: CNC1CC(Nc2ccc(C(C)(C)C)cc2)C1] | 195 |
| 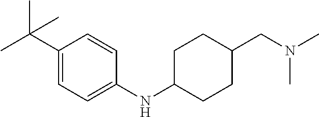 [SMILES: CN(C)CC1CCC(Nc2ccc(C(C)(C)C)cc2)CC1] | 196 |
| 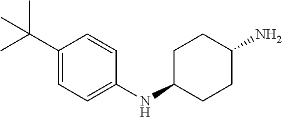 [SMILES: CC(C)(C)c1ccc(N[C@H]2CC[C@H](N)CC2)cc1] | 197 |
| 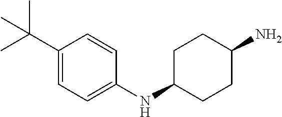 [SMILES: CC(C)(C)c1ccc(N[C@H]2CC[C@@H](N)CC2)cc1] | 198 |
| 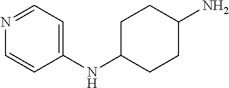 [SMILES: NC1CCC(Nc2ccncc2)CC1] | 199 |
| 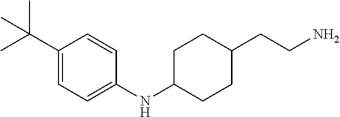 [SMILES: CC(C)(C)c1ccc(NC2CCC(CCN)CC2)cc1] | 200A/B |
| 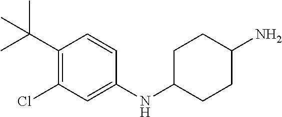 [SMILES: CC(C)(C)c1ccc(NC2CCC(N)CC2)cc1Cl] | 201 |
| 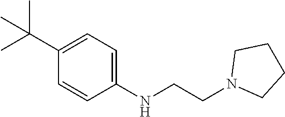 [SMILES: CC(C)(C)c1ccc(NCCN2CCCC2)cc1] | 202 |
| 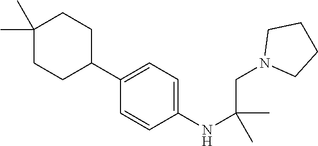 [SMILES: CC1(C)CCC(c2ccc(NC(C)(C)CN3CCCC3)cc2)CC1] | 203 |
| 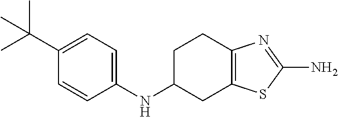 [SMILES: CC(C)(C)c1ccc(NC2CCc3nc(N)sc3C2)cc1] | 204 |
| 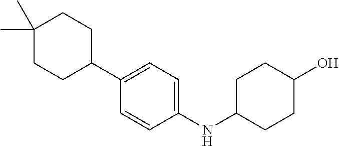 [SMILES: CC1(C)CCC(c2ccc(NC3CCC(O)CC3)cc2)CC1] | 205A/B |
| 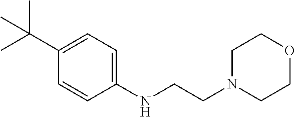 [SMILES: CC(C)(C)c1ccc(NCCN2CCOCC2)cc1] | 206 |
| 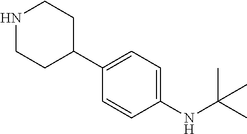 [SMILES: CC(C)(C)Nc1ccc(C2CCNCC2)cc1] | 207 |
| 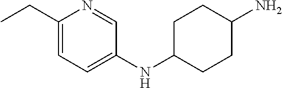 [SMILES: CCc1ccc(NC2CCC(N)CC2)cn1] | 208 |
| 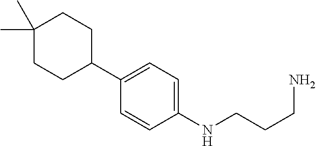 [SMILES: CC1(C)CCC(c2ccc(NCCCN)cc2)CC1] | 209 |
| 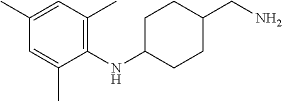 [SMILES: Cc1cc(C)c(NC2CCC(CN)CC2)c(C)c1] | 210 |
| 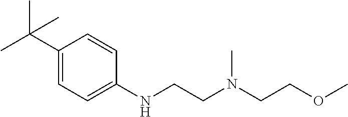 [SMILES: COCCN(C)CCNc1ccc(C(C)(C)C)cc1] | 211 |
| 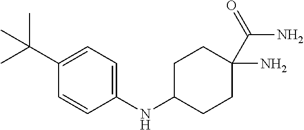 [SMILES: CC(C)(C)c1ccc(NC2CCC(N)(C(N)=O)CC2)cc1] | 212 |
| 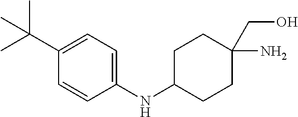 [SMILES: CC(C)(C)c1ccc(NC2CCC(N)(CO)CC2)cc1] | 213A/B |
| 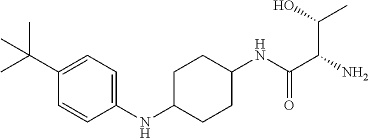 [SMILES: C[C@@H](O)[C@H](N)C(=O)NC1CCC(Nc2ccc(C(C)(C)C)cc2)CC1] | 214 |
| 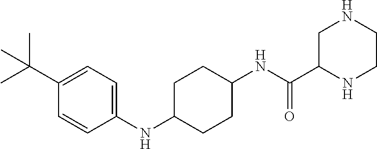 [SMILES: CC(C)(C)c1ccc(NC2CCC(NC(=O)C3CNCCN3)CC2)cc1] | 215 |
| 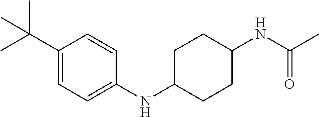 [SMILES: CC(=O)NC1CCC(Nc2ccc(C(C)(C)C)cc2)CC1] | 216A/B |
| 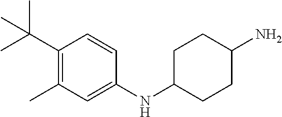 [SMILES: Cc1cc(NC2CCC(N)CC2)ccc1C(C)(C)C] | 217 |
| 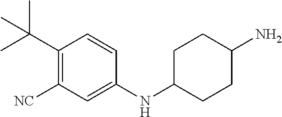 [SMILES: CC(C)(C)c1ccc(NC2CCC(N)CC2)cc1C#N] | 218 |
| 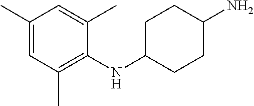 [SMILES: Cc1cc(C)c(NC2CCC(N)CC2)c(C)c1] | 219 |
| 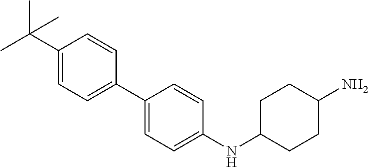 [SMILES: CC(C)(C)c1ccc(-c2ccc(NC3CCC(N)CC3)cc2)cc1] | 220 |
| 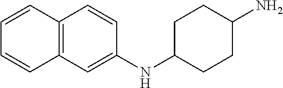 [SMILES: NC1CCC(Nc2ccc3ccccc3c2)CC1] | 221 |
| 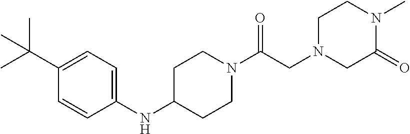 [SMILES: CN1CCN(CC(=O)N2CCC(Nc3ccc(C(C)(C)C)cc3)CC2)CC1=O] | 222 |
| 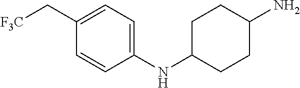 [SMILES: NC1CCC(Nc2ccc(CC(F)(F)F)cc2)CC1] | 223 |
| 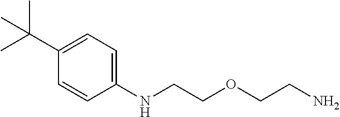 [SMILES: CC(C)(C)c1ccc(NCCOCCN)cc1] | 224 |
| 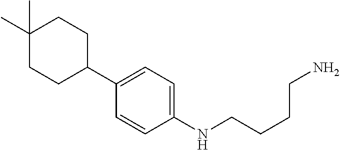 [SMILES: CC1(C)CCC(c2ccc(NCCCCN)cc2)CC1] | 225 |
| 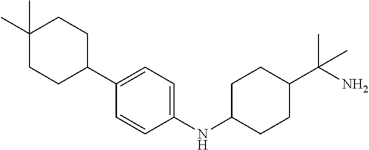 [SMILES: CC1(C)CCC(c2ccc(NC3CCC(C(C)(C)N)CC3)cc2)CC1] | 226 |
| 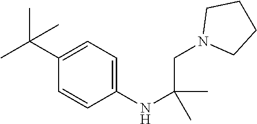 [SMILES: CC(C)(CN1CCCC1)Nc1ccc(C(C)(C)C)cc1] | 227 |
| 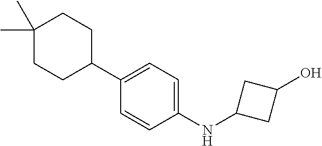 [SMILES: CC1(C)CCC(c2ccc(NC3CC(O)C3)cc2)CC1] | 228A/B |
| 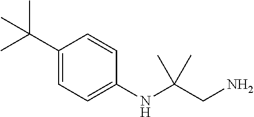 [SMILES: CC(C)(CN)Nc1ccc(C(C)(C)C)cc1] | 229 |
| 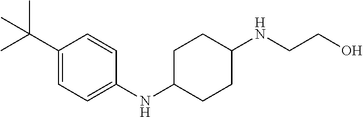 [SMILES: CC(C)(C)c1ccc(NC2CCC(NCCO)CC2)cc1] | 230 |
| 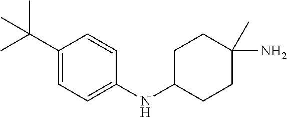 [SMILES: CC1(N)CCC(Nc2ccc(C(C)(C)C)cc2)CC1] | 231A/B |
| 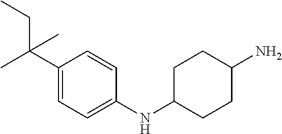 [SMILES: CCC(C)(C)c1ccc(NC2CCC(N)CC2)cc1] | 232A/B |
| 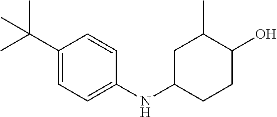 [SMILES: CC1CC(Nc2ccc(C(C)(C)C)cc2)CCC1O] | 233A/B |
| 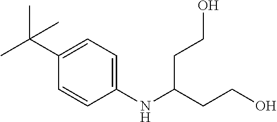 [SMILES: CC(C)(C)c1ccc(NC(CCO)CCO)cc1] | 234 |
| 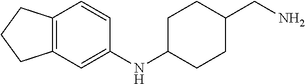 [SMILES: NCC1CCC(Nc2ccc3c(c2)CCC3)CC1] | 235 |
| 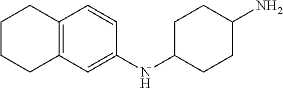 [SMILES: NC1CCC(Nc2ccc3c(c2)CCCC3)CC1] | 236 |
| 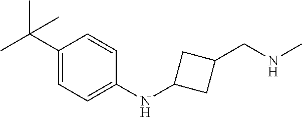 [SMILES: CNCC1CC(Nc2ccc(C(C)(C)C)cc2)C1] | 237 |
| 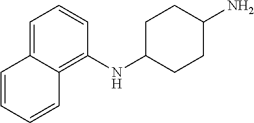 [SMILES: NC1CCC(Nc2cccc3ccccc23)CC1] | 238 |
| 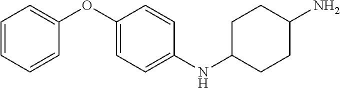 [SMILES: NC1CCC(Nc2ccc(Oc3ccccc3)cc2)CC1] | 239 |
| 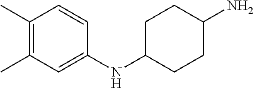 [SMILES: Cc1ccc(NC2CCC(N)CC2)cc1C] | 240 |
| 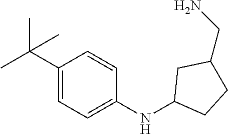 [SMILES: CC(C)(C)c1ccc(NC2CCC(CN)C2)cc1] | 241 |
| 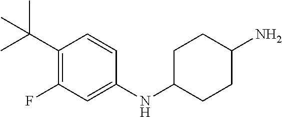 [SMILES: CC(C)(C)c1ccc(NC2CCC(N)CC2)cc1F] | 242A/B |
| 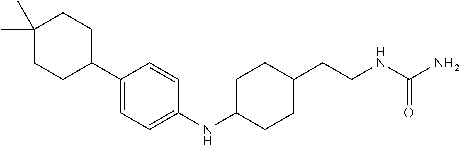 [SMILES: CC1(C)CCC(c2ccc(NC3CCC(CCNC(N)=O)CC3)cc2)CC1] | 243 |
| 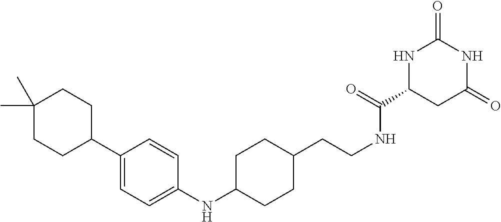 [SMILES: CC1(C)CCC(c2ccc(NC3CCC(CCNC(=O)[C@H]4CC(=O)NC(=O)N4)CC3)cc2)CC1] | 244A/B |
| 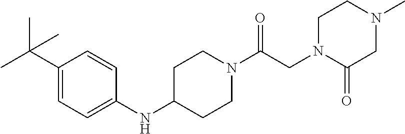 [SMILES: CN1CCN(CC(=O)N2CCC(Nc3ccc(C(C)(C)C)cc3)CC2)C(=O)C1] | 245 |
| 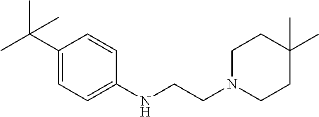 [SMILES: CC1(C)CCN(CCNc2ccc(C(C)(C)C)cc2)CC1] | 246 |
| 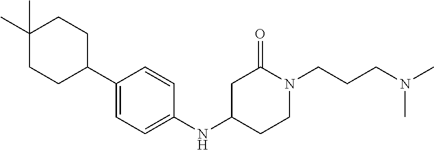 | 247 |
| 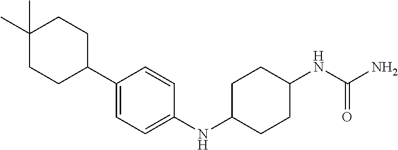 [SMILES: CC1(C)CCC(c2ccc(NC3CCC(NC(N)=O)CC3)cc2)CC1] | 248 |
| 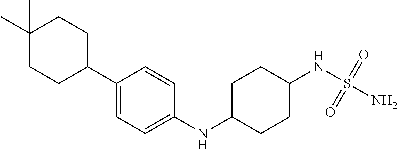 [SMILES: CC1(C)CCC(c2ccc(NC3CCC(NS(N)(=O)=O)CC3)cc2)CC1] | 249 |
| 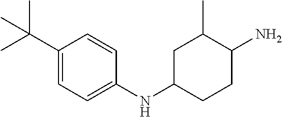 [SMILES: CC1CC(Nc2ccc(C(C)(C)C)cc2)CCC1N] | 250A/B |
| 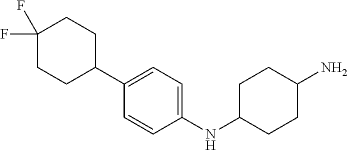 [SMILES: NC1CCC(Nc2ccc(C3CCC(F)(F)CC3)cc2)CC1] | 251A/B |
| 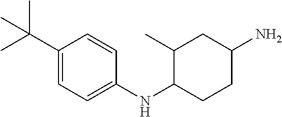 [SMILES: CC1CC(N)CCC1Nc1ccc(C(C)(C)C)cc1] | 252A/B |
| 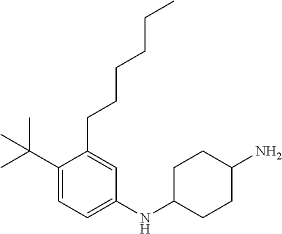 [SMILES: CCCCCCc1cc(NC2CCC(N)CC2)ccc1C(C)(C)C] | 253A/B |
| 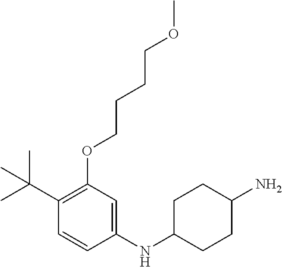 [SMILES: COCCCCOc1cc(NC2CCC(N)CC2)ccc1C(C)(C)C] | 254 |
| 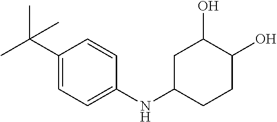 [SMILES: CC(C)(C)c1ccc(NC2CCC(O)C(O)C2)cc1] | 255 |
| 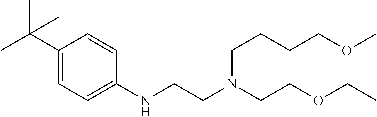 [SMILES: CCOCCN(CCCCOC)CCNc1ccc(C(C)(C)C)cc1] | 256 |
| 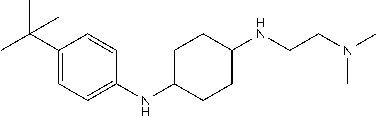 [SMILES: CN(C)CCNC1CCC(Nc2ccc(C(C)(C)C)cc2)CC1] | 257 |
| 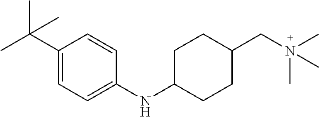 | 258 |
| 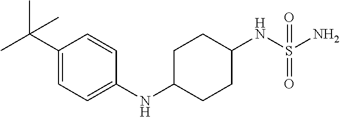 [SMILES: CC(C)(C)c1ccc(NC2CCC(NS(N)(=O)=O)CC2)cc1] | 259A/B |
| 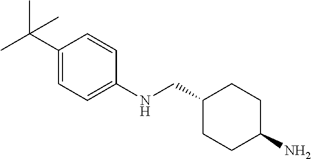 [SMILES: CC(C)(C)c1ccc(NC[C@H]2CC[C@H](N)CC2)cc1] | 260 |
| 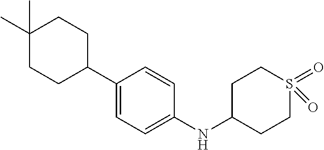 [SMILES: CC1(C)CCC(c2ccc(NC3CCS(=O)(=O)CC3)cc2)CC1] | 261 |
| 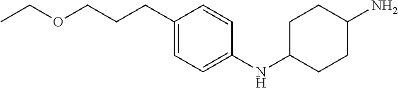 [SMILES: CCOCCCc1ccc(NC2CCC(N)CC2)cc1] | 262 |
| 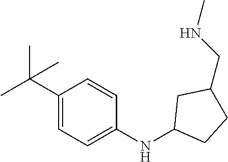 [SMILES: CNCC1CCC(Nc2ccc(C(C)(C)C)cc2)C1] | 263 |
| 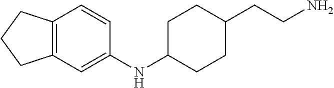 [SMILES: NCCC1CCC(Nc2ccc3c(c2)CCC3)CC1] | 264 |
| 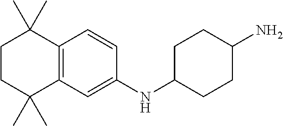 [SMILES: CC1(C)CCC(C)(C)c2cc(NC3CCC(N)CC3)ccc21] | 265 |
| 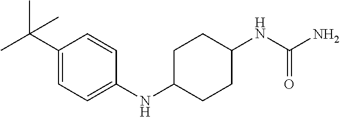 [SMILES: CC(C)(C)c1ccc(NC2CCC(NC(N)=O)CC2)cc1] | 266A/B |
| 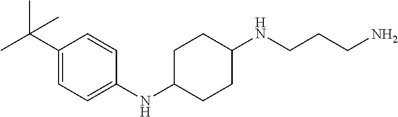 [SMILES: CC(C)(C)c1ccc(NC2CCC(NCCCN)CC2)cc1] | 267 |
| 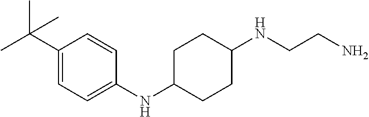 [SMILES: CC(C)(C)c1ccc(NC2CCC(NCCN)CC2)cc1] | 268 |
| 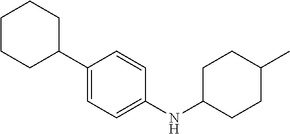 [SMILES: CC1CCC(Nc2ccc(C3CCCCC3)cc2)CC1] | 269 |
| 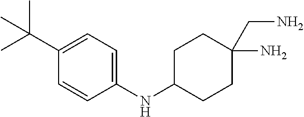 [SMILES: CC(C)(C)c1ccc(NC2CCC(N)(CN)CC2)cc1] | 270 |
| 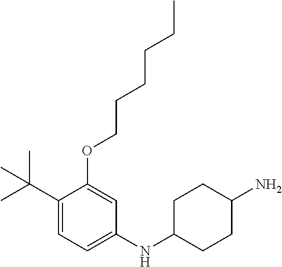 [SMILES: CCCCCCOc1cc(NC2CCC(N)CC2)ccc1C(C)(C)C] | 271 |
| 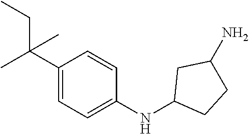 [SMILES: CCC(C)(C)c1ccc(NC2CCC(N)C2)cc1] | 272 |
| 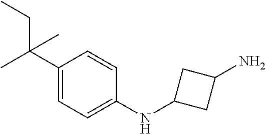 [SMILES: CCC(C)(C)c1ccc(NC2CC(N)C2)cc1] | 273 |
| 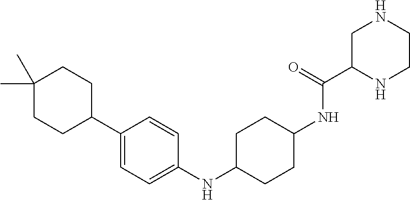 [SMILES: CC1(C)CCC(c2ccc(NC3CCC(NC(=O)C4CNCCN4)CC3)cc2)CC1] | 274 |
| 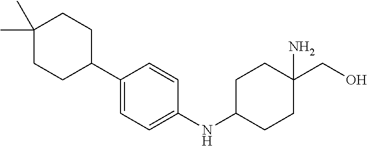 [SMILES: CC1(C)CCC(c2ccc(NC3CCC(N)(CO)CC3)cc2)CC1] | 275A/B |
| 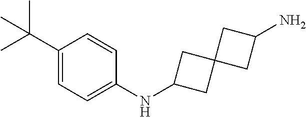 [SMILES: CC(C)(C)c1ccc(NC2CC3(CC(N)C3)C2)cc1] | 276 |
| 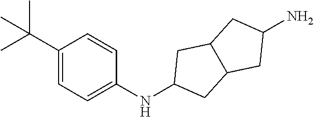 [SMILES: CC(C)(C)c1ccc(NC2CC3CC(N)CC3C2)cc1] | 277 |
| 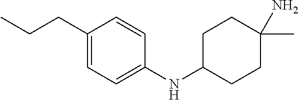 [SMILES: CCCc1ccc(NC2CCC(C)(N)CC2)cc1] | 278 |
| 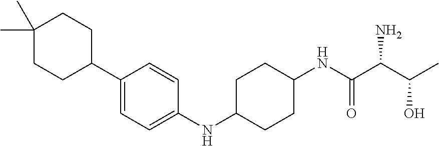 [SMILES: C[C@H](O)[C@@H](N)C(=O)NC1CCC(Nc2ccc(C3CCC(C)(C)CC3)cc2)CC1] | 279 |
| 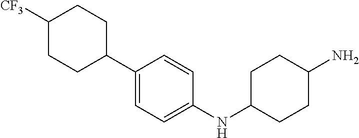 [SMILES: NC1CCC(Nc2ccc(C3CCC(C(F)(F)F)CC3)cc2)CC1] | 280 |
| 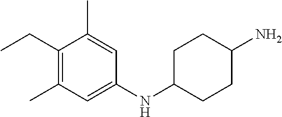 [SMILES: CCc1c(C)cc(NC2CCC(N)CC2)cc1C] | 281 |
| 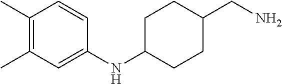 [SMILES: Cc1ccc(NC2CCC(CN)CC2)cc1C] | 282 |
| 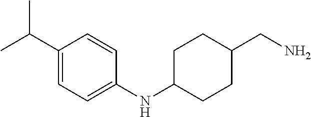 [SMILES: CC(C)c1ccc(NC2CCC(CN)CC2)cc1] | 283 |
| 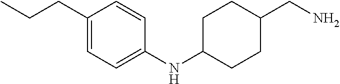 [SMILES: CCCc1ccc(NC2CCC(CN)CC2)cc1] | 284 |
| 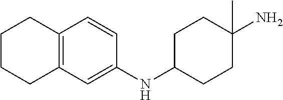 [SMILES: CC1(N)CCC(Nc2ccc3c(c2)CCCC3)CC1] | 285 |
| 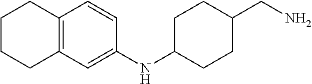 [SMILES: NCC1CCC(Nc2ccc3c(c2)CCCC3)CC1] | 286 |
| 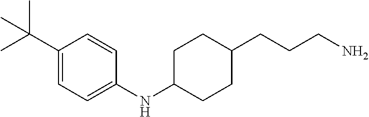 [SMILES: CC(C)(C)c1ccc(NC2CCC(CCCN)CC2)cc1] | 287 |
| 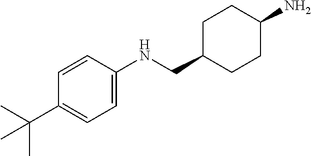 [SMILES: CC(C)(C)c1ccc(NC[C@H]2CC[C@@H](N)CC2)cc1] | 288 |
| 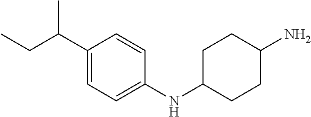 [SMILES: CCC(C)c1ccc(NC2CCC(N)CC2)cc1] | 289A/B |
| 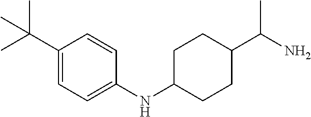 [SMILES: CC(N)C1CCC(Nc2ccc(C(C)(C)C)cc2)CC1] | 290 |
| 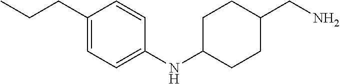 [SMILES: CCCc1ccc(NC2CCC(CN)CC2)cc1] | 291 |
| 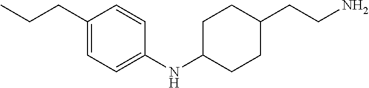 [SMILES: CCCc1ccc(NC2CCC(CCN)CC2)cc1] | 292 |
| 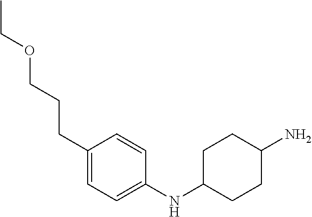 [SMILES: CCOCCCc1ccc(NC2CCC(N)CC2)cc1] | 293 |
| 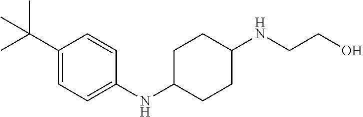 [SMILES: CC(C)(C)c1ccc(NC2CCC(NCCO)CC2)cc1] | 294A/B |
| 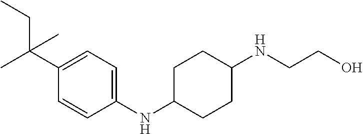 [SMILES: CCC(C)(C)c1ccc(NC2CCC(NCCO)CC2)cc1] | 295A/B |
| 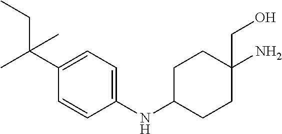 [SMILES: CCC(C)(C)c1ccc(NC2CCC(N)(CO)CC2)cc1] | 296A/B |
| 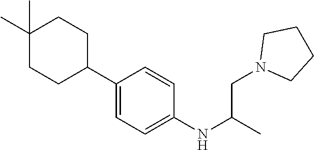 [SMILES: CC(CN1CCCC1)Nc1ccc(C2CCC(C)(C)CC2)cc1] | 297 |
| 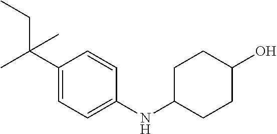 [SMILES: CCC(C)(C)c1ccc(NC2CCC(O)CC2)cc1] | 298 |
| 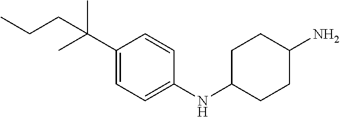 [SMILES: CCCC(C)(C)c1ccc(NC2CCC(N)CC2)cc1] | 299A/B |
| 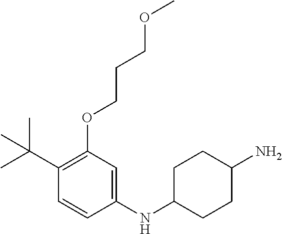 [SMILES: COCCCOc1cc(NC2CCC(N)CC2)ccc1C(C)(C)C] | 300A/B |
| 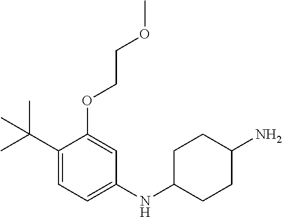 [SMILES: COCCOc1cc(NC2CCC(N)CC2)ccc1C(C)(C)C] | 301A/B |
| 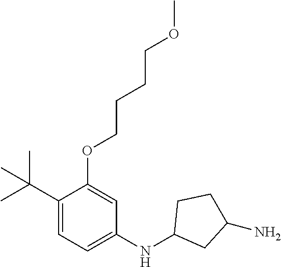 [SMILES: COCCCCOc1cc(NC2CCC(N)C2)ccc1C(C)(C)C] | 302 |
| 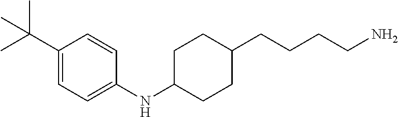 [SMILES: CC(C)(C)c1ccc(NC2CCC(CCCCN)CC2)cc1] | 303 |
| 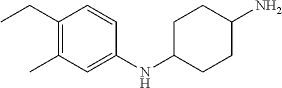 [SMILES: CCc1ccc(NC2CCC(N)CC2)cc1C] | 304 |
| 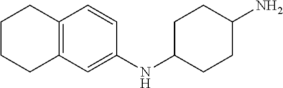 [SMILES: NC1CCC(Nc2ccc3c(c2)CCCC3)CC1] | 305A/B |
| 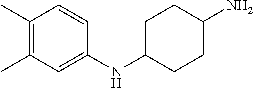 [SMILES: Cc1ccc(NC2CCC(N)CC2)cc1C] | 306A/B |
| 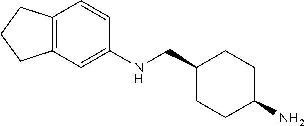 [SMILES: N[C@H]1CC[C@@H](CNc2ccc3c(c2)CCC3)CC1] | 307 |
| 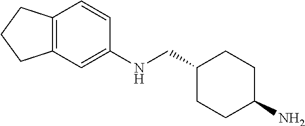 [SMILES: N[C@H]1CC[C@H](CNc2ccc3c(c2)CCC3)CC1] | 308 |
| 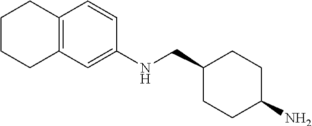 [SMILES: N[C@H]1CC[C@@H](CNc2ccc3c(c2)CCCC3)CC1] | 309 |
| 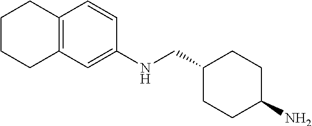 [SMILES: N[C@H]1CC[C@H](CNc2ccc3c(c2)CCCC3)CC1] | 310 |
| 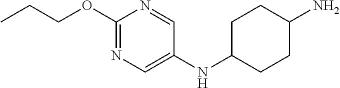 [SMILES: CCCOc1ncc(NC2CCC(N)CC2)cn1] | 311 |
| 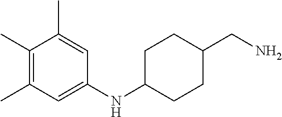 [SMILES: Cc1cc(NC2CCC(CN)CC2)cc(C)c1C] | 312 |
| 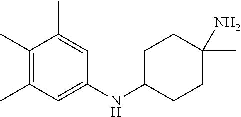 | 313A/B |
| 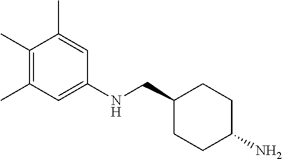 [SMILES: Cc1cc(NC[C@H]2CC[C@H](N)CC2)cc(C)c1C] | 314 |
| 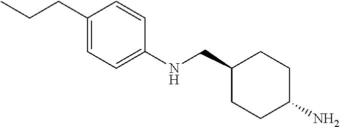 [SMILES: CCCc1ccc(NC[C@H]2CC[C@H](N)CC2)cc1] | 315 |
| 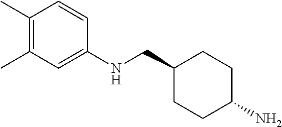 [SMILES: Cc1ccc(NC[C@H]2CC[C@H](N)CC2)cc1C] | 316 |
| 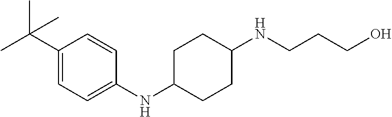 [SMILES: CC(C)(C)c1ccc(NC2CCC(NCCCO)CC2)cc1] | 317 |
| 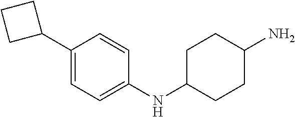 [SMILES: NC1CCC(Nc2ccc(C3CCC3)cc2)CC1] | 318A/B |
| 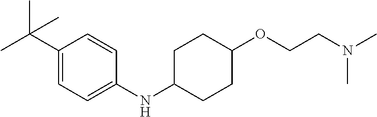 [SMILES: CN(C)CCOC1CCC(Nc2ccc(C(C)(C)C)cc2)CC1] | 319A/B |
| 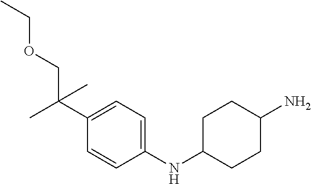 [SMILES: CCOCC(C)(C)c1ccc(NC2CCC(N)CC2)cc1] | 320A/B |
| 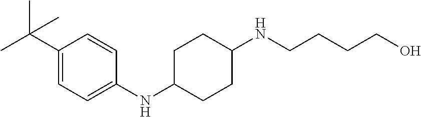 [SMILES: CC(C)(C)c1ccc(NC2CCC(NCCCCO)CC2)cc1] | 321 |
| 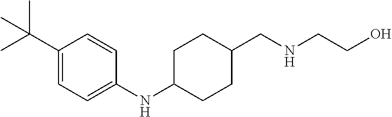 [SMILES: CC(C)(C)c1ccc(NC2CCC(CNCCO)CC2)cc1] | 322 |
| 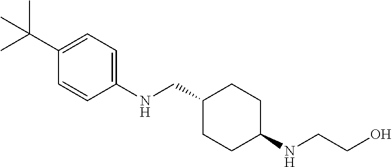 [SMILES: CC(C)(C)c1ccc(NC[C@H]2CC[C@H](NCCO)CC2)cc1] | 323 |
| 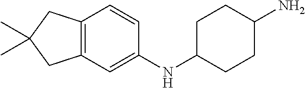 [SMILES: CC1(C)Cc2ccc(NC3CCC(N)CC3)cc2C1] | 324 |
| 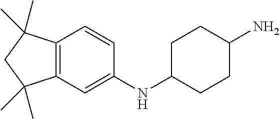 [SMILES: CC1(C)CC(C)(C)c2cc(NC3CCC(N)CC3)ccc21] | 325A/B |
| 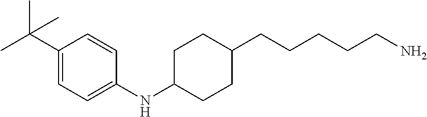 [SMILES: CC(C)(C)c1ccc(NC2CCC(CCCCCN)CC2)cc1] | 326 |
| 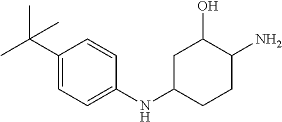 [SMILES: CC(C)(C)c1ccc(NC2CCC(N)C(O)C2)cc1] | 327 |
| 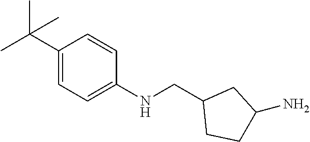 [SMILES: CC(C)(C)c1ccc(NCC2CCC(N)C2)cc1] | 328 |
| 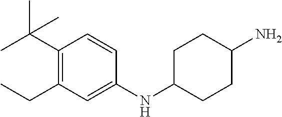 [SMILES: CCc1cc(NC2CCC(N)CC2)ccc1C(C)(C)C] | 329 |
| 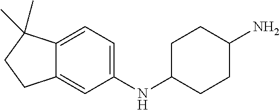 [SMILES: CC1(C)CCc2cc(NC3CCC(N)CC3)ccc21] | 330A/B |
| 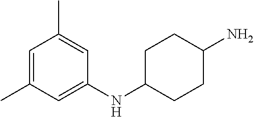 [SMILES: Cc1cc(C)cc(NC2CCC(N)CC2)c1] | 331 |
| 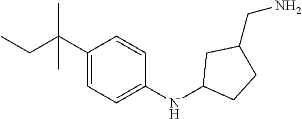 [SMILES: CCC(C)(C)c1ccc(NC2CCC(CN)C2)cc1] | 332 |
| 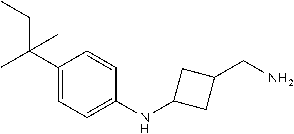 [SMILES: CCC(C)(C)c1ccc(NC2CC(CN)C2)cc1] | 333 |
| 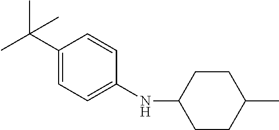 [SMILES: CC1CCC(Nc2ccc(C(C)(C)C)cc2)CC1] | 334 |
| 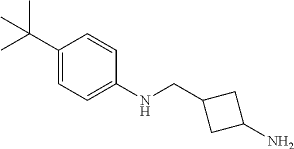 [SMILES: CC(C)(C)c1ccc(NCC2CC(N)C2)cc1] | 335A/B |
| 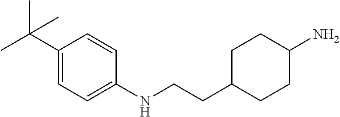 [SMILES: CC(C)(C)c1ccc(NCCC2CCC(N)CC2)cc1] | 336A/B |
| 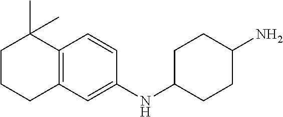 [SMILES: CC1(C)CCCc2cc(NC3CCC(N)CC3)ccc21] | 337A/B |
| 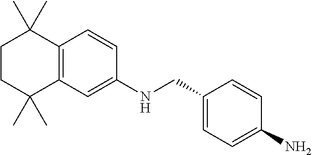 | 338 |
| 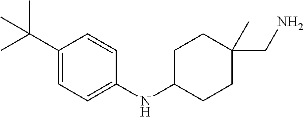 [SMILES: CC1(CN)CCC(Nc2ccc(C(C)(C)C)cc2)CC1] | 339 |
| 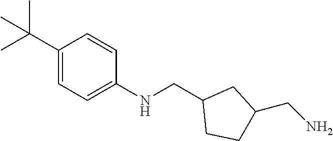 [SMILES: CC(C)(C)c1ccc(NCC2CCC(CN)C2)cc1] | 340 |
| 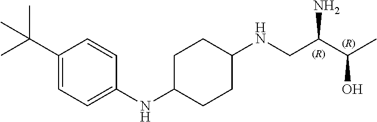 | 341 |
| 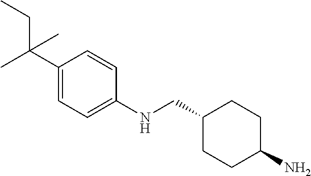 [SMILES: CCC(C)(C)c1ccc(NC[C@H]2CC[C@H](N)CC2)cc1] | 342 |
| 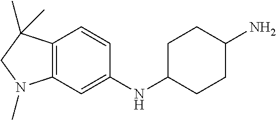 [SMILES: CN1CC(C)(C)c2ccc(NC3CCC(N)CC3)cc21] | 343 |
| 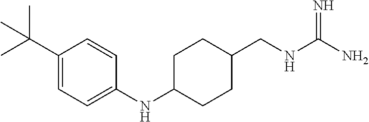 [SMILES: CC(C)(C)c1ccc(NC2CCC(CNC(=N)N)CC2)cc1] | 344 |
| 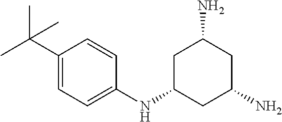 [SMILES: CC(C)(C)c1ccc(N[C@@H]2C[C@H](N)C[C@H](N)C2)cc1] | 345 |
| 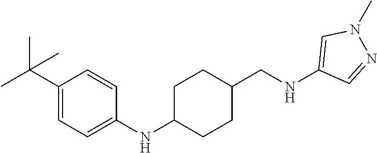 [SMILES: Cn1cc(NCC2CCC(Nc3ccc(C(C)(C)C)cc3)CC2)cn1] | 346 |
| 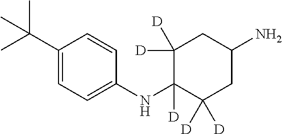 [SMILES: [2H]C1([2H])CC(N)CC([2H])([2H])C1([2H])Nc1ccc(C(C)(C)C)cc1] | 347 |
| 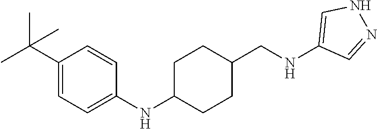 [SMILES: CC(C)(C)c1ccc(NC2CCC(CNc3cn[nH]c3)CC2)cc1] | 348 |
| 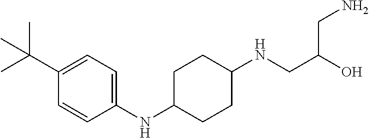 [SMILES: CC(C)(C)c1ccc(NC2CCC(NCC(O)CN)CC2)cc1] | 349A/B |
| 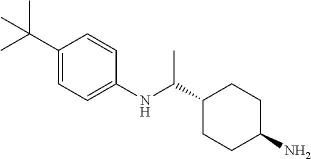 [SMILES: CC(Nc1ccc(C(C)(C)C)cc1)[C@H]1CC[C@H](N)CC1] | 350 |
| 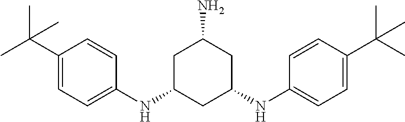 [SMILES: CC(C)(C)c1ccc(N[C@@H]2C[C@H](N)C[C@H](Nc3ccc(C(C)(C)C)cc3)C2)cc1] | 351 |
| 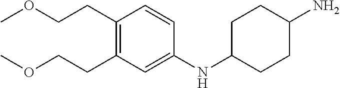 [SMILES: COCCc1ccc(NC2CCC(N)CC2)cc1CCOC] | 352 |
| 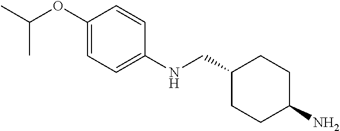 [SMILES: CC(C)Oc1ccc(NC[C@H]2CC[C@H](N)CC2)cc1] | 353 |
| 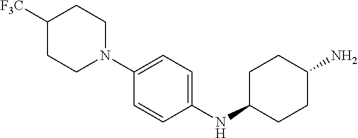 [SMILES: N[C@H]1CC[C@H](Nc2ccc(N3CCC(C(F)(F)F)CC3)cc2)CC1] | 354 |
| 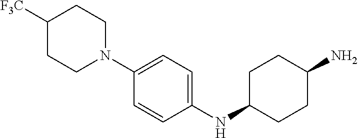 [SMILES: N[C@H]1CC[C@@H](Nc2ccc(N3CCC(C(F)(F)F)CC3)cc2)CC1] | 355 |
| 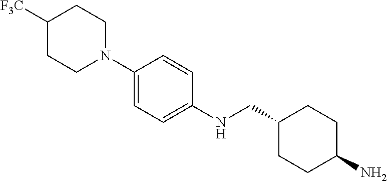 [SMILES: N[C@H]1CC[C@H](CNc2ccc(N3CCC(C(F)(F)F)CC3)cc2)CC1] | 356 |
| 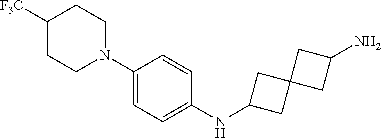 [SMILES: NC1CC2(C1)CC(Nc1ccc(N3CCC(C(F)(F)F)CC3)cc1)C2] | 357 |
| 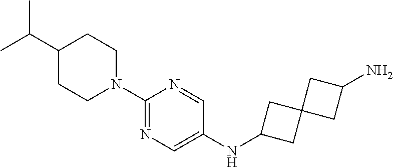 [SMILES: CC(C)C1CCN(c2ncc(NC3CC4(CC(N)C4)C3)cn2)CC1] | 358 |
| 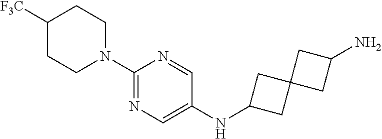 [SMILES: NC1CC2(C1)CC(Nc1cnc(N3CCC(C(F)(F)F)CC3)nc1)C2] | 359 |
| 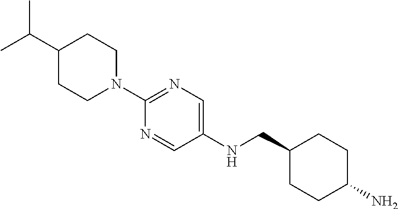 [SMILES: CC(C)C1CCN(c2ncc(NC[C@H]3CC[C@H](N)CC3)cn2)CC1] | 360 |
| 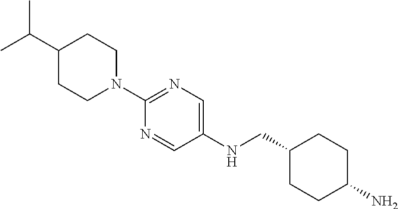 [SMILES: CC(C)C1CCN(c2ncc(NC[C@H]3CC[C@@H](N)CC3)cn2)CC1] | 361 |
| 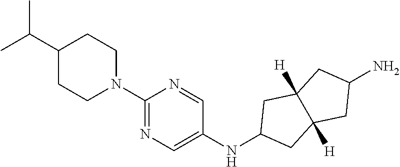 [SMILES: CC(C)C1CCN(c2ncc(NC3C[C@H]4CC(N)C[C@H]4C3)cn2)CC1] | 362 |
| 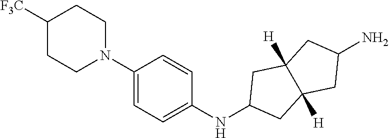 [SMILES: NC1C[C@@H]2CC(Nc3ccc(N4CCC(C(F)(F)F)CC4)cc3)C[C@@H]2C1] | 363 |
| 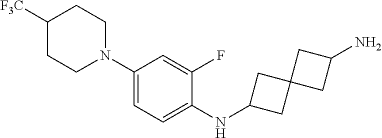 [SMILES: NC1CC2(C1)CC(Nc1ccc(N3CCC(C(F)(F)F)CC3)cc1F)C2] | 364 |
| 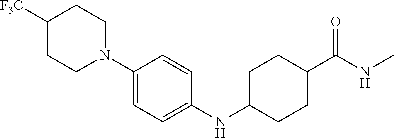 [SMILES: CNC(=O)C1CCC(Nc2ccc(N3CCC(C(F)(F)F)CC3)cc2)CC1] | 365 |
| 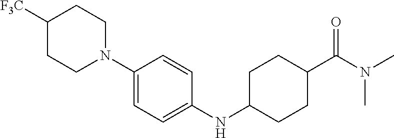 [SMILES: CN(C)C(=O)C1CCC(Nc2ccc(N3CCC(C(F)(F)F)CC3)cc2)CC1] | 366 |
| 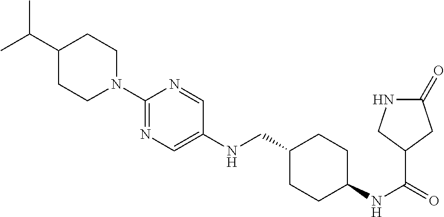 [SMILES: CC(C)C1CCN(c2ncc(NC[C@H]3CC[C@H](NC(=O)C4CNC(=O)C4)CC3)cn2)CC1] | 367 |
| 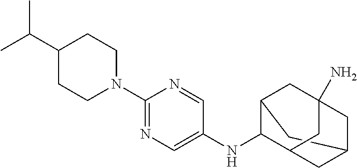 [SMILES: CC(C)C1CCN(c2ncc(NC3C4CC5CC3CC(N)(C5)C4)cn2)CC1] | 368 |
| 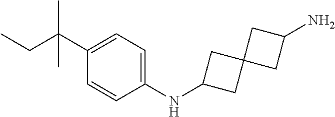 [SMILES: CCC(C)(C)c1ccc(NC2CC3(CC(N)C3)C2)cc1] | 369 |
| 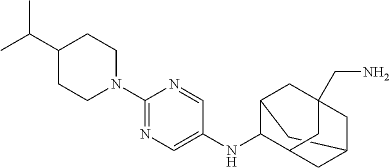 [SMILES: CC(C)C1CCN(c2ncc(NC3C4CC5CC3CC(CN)(C5)C4)cn2)CC1] | 370 |
| 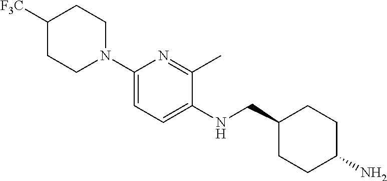 [SMILES: Cc1nc(N2CCC(C(F)(F)F)CC2)ccc1NC[C@H]1CC[C@H](N)CC1] | 371 |
| 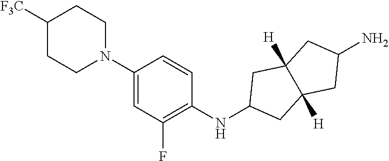 [SMILES: NC1C[C@@H]2CC(Nc3ccc(N4CCC(C(F)(F)F)CC4)cc3F)C[C@@H]2C1] | 372 |
| 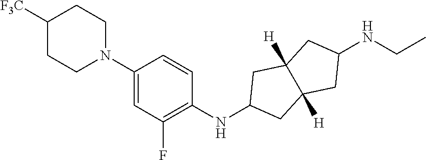 [SMILES: CCNC1C[C@@H]2CC(Nc3ccc(N4CCC(C(F)(F)F)CC4)cc3F)C[C@@H]2C1] | 373 |
| 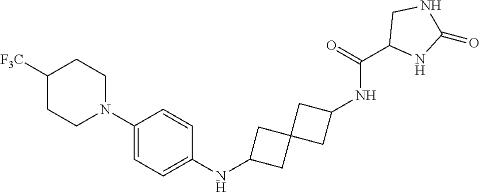 [SMILES: O=C1NCC(C(=O)NC2CC3(C2)CC(Nc2ccc(N4CCC(C(F)(F)F)CC4)cc2)C3)N1] | 374 |
| 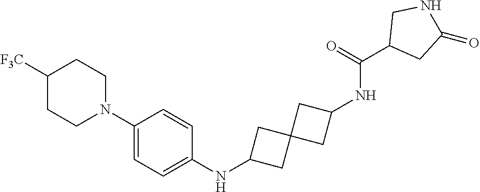 [SMILES: O=C1CC(C(=O)NC2CC3(C2)CC(Nc2ccc(N4CCC(C(F)(F)F)CC4)cc2)C3)CN1] | 375 |
| 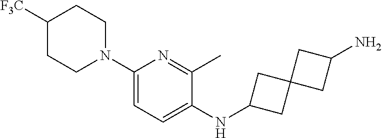 [SMILES: Cc1nc(N2CCC(C(F)(F)F)CC2)ccc1NC1CC2(CC(N)C2)C1] | 376 |
| 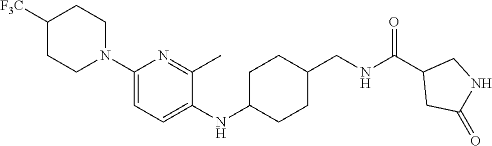 [SMILES: Cc1nc(N2CCC(C(F)(F)F)CC2)ccc1NC1CCC(CNC(=O)C2CNC(=O)C2)CC1] | 377 |
| 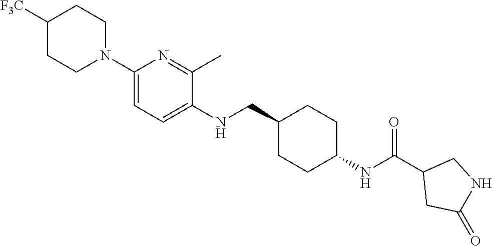 [SMILES: Cc1nc(N2CCC(C(F)(F)F)CC2)ccc1NC[C@H]1CC[C@H](NC(=O)C2CNC(=O)C2)CC1] | 378 |
| 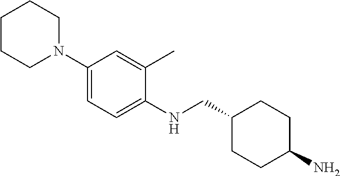 [SMILES: Cc1cc(N2CCCCC2)ccc1NC[C@H]1CC[C@H](N)CC1] | 379 |
| 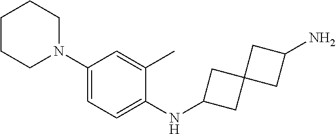 [SMILES: Cc1cc(N2CCCCC2)ccc1NC1CC2(CC(N)C2)C1] | 380 |
| 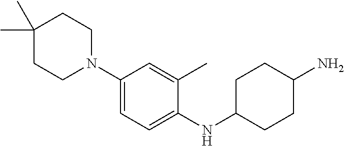 [SMILES: Cc1cc(N2CCC(C)(C)CC2)ccc1NC1CCC(N)CC1] | 381 |
| 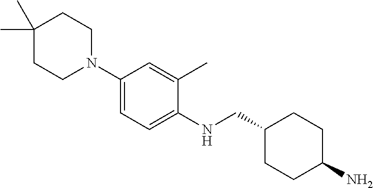 [SMILES: Cc1cc(N2CCC(C)(C)CC2)ccc1NC[C@H]1CC[C@H](N)CC1] | 382 |
| 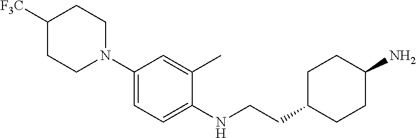 [SMILES: Cc1cc(N2CCC(C(F)(F)F)CC2)ccc1NCC[C@H]1CC[C@H](N)CC1] | 383 |
| 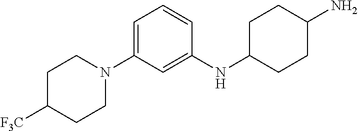 [SMILES: NC1CCC(Nc2cccc(N3CCC(C(F)(F)F)CC3)c2)CC1] | 384 |
| 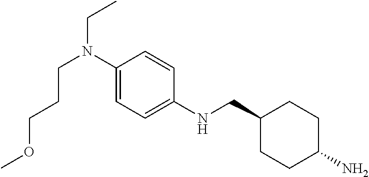 [SMILES: CCN(CCCOC)c1ccc(NC[C@H]2CC[C@H](N)CC2)cc1] | 385 |
| 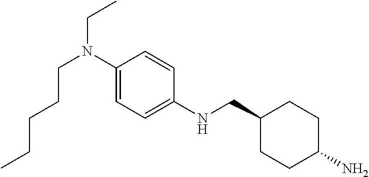 [SMILES: CCCCCN(CC)c1ccc(NC[C@H]2CC[C@H](N)CC2)cc1] | 386 |
| 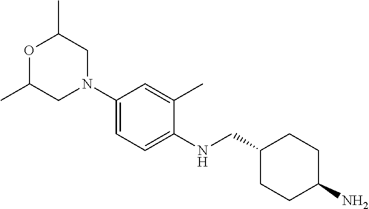 [SMILES: Cc1cc(N2CC(C)OC(C)C2)ccc1NC[C@H]1CC[C@H](N)CC1] | 387 |
| 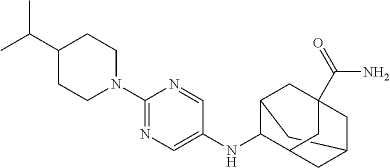 [SMILES: CC(C)C1CCN(c2ncc(NC3C4CC5CC3CC(C(N)=O)(C5)C4)cn2)CC1] | 388 |
| 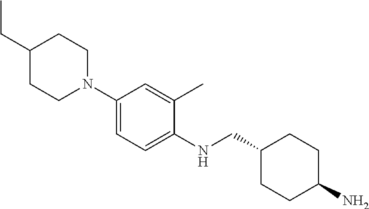 [SMILES: CCC1CCN(c2ccc(NC[C@H]3CC[C@H](N)CC3)c(C)c2)CC1] | 389 |
| 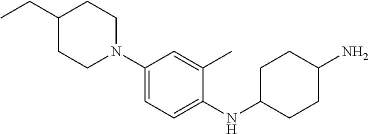 [SMILES: CCC1CCN(c2ccc(NC3CCC(N)CC3)c(C)c2)CC1] | 390 |
| 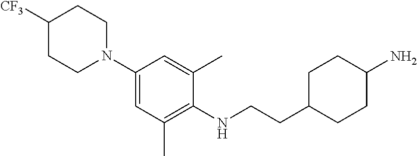 [SMILES: Cc1cc(N2CCC(C(F)(F)F)CC2)cc(C)c1NCCC1CCC(N)CC1] | 391 |
| 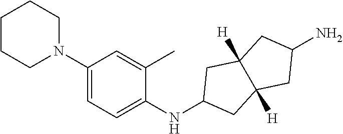 [SMILES: Cc1cc(N2CCCCC2)ccc1NC1C[C@H]2CC(N)C[C@H]2C1] | 392 |
| 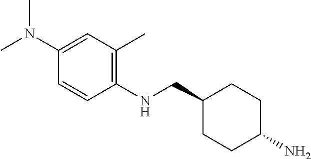 [SMILES: Cc1cc(N(C)C)ccc1NC[C@H]1CC[C@H](N)CC1] | 393 |
| 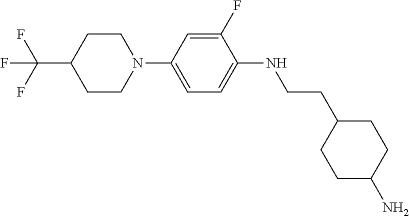 [SMILES: NC1CCC(CCNc2ccc(N3CCC(C(F)(F)F)CC3)cc2F)CC1] | 394 |
| 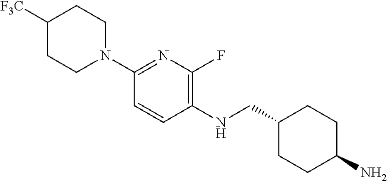 [SMILES: N[C@H]1CC[C@H](CNc2ccc(N3CCC(C(F)(F)F)CC3)nc2F)CC1] | 395 |
| 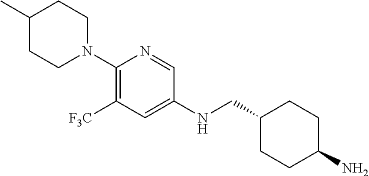 [SMILES: CC1CCN(c2ncc(NC[C@H]3CC[C@H](N)CC3)cc2C(F)(F)F)CC1] | 396 |
| 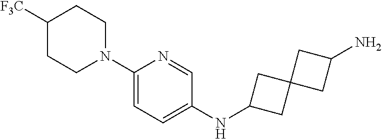 [SMILES: NC1CC2(C1)CC(Nc1ccc(N3CCC(C(F)(F)F)CC3)nc1)C2] | 397 |
| 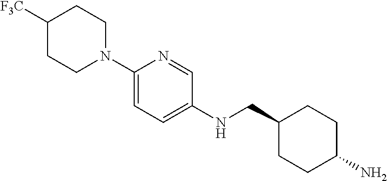 [SMILES: N[C@H]1CC[C@H](CNc2ccc(N3CCC(C(F)(F)F)CC3)nc2)CC1] | 398 |
| 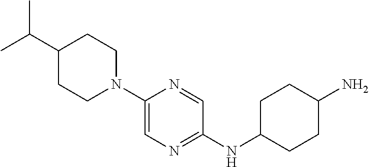 [SMILES: CC(C)C1CCN(c2cnc(NC3CCC(N)CC3)cn2)CC1] | 399 |
| 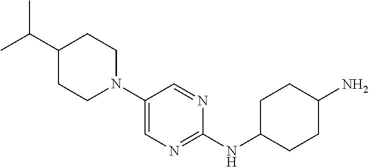 [SMILES: CC(C)C1CCN(c2cnc(NC3CCC(N)CC3)nc2)CC1] | 400 |
| 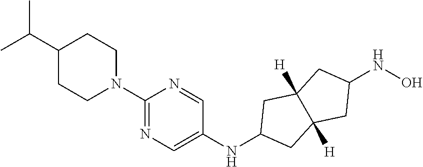 [SMILES: CC(C)C1CCN(c2ncc(NC3C[C@H]4CC(NO)C[C@H]4C3)cn2)CC1] | 401 |
| 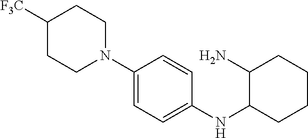 [SMILES: NC1CCCCC1Nc1ccc(N2CCC(C(F)(F)F)CC2)cc1] | 402 |
| 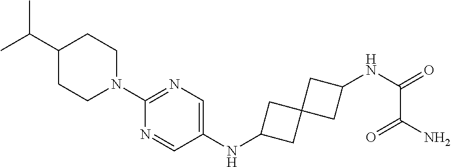 [SMILES: CC(C)C1CCN(c2ncc(NC3CC4(CC(NC(=O)C(N)=O)C4)C3)cn2)CC1] | 403 |
| 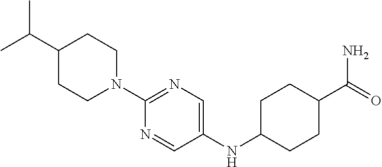 [SMILES: CC(C)C1CCN(c2ncc(NC3CCC(C(N)=O)CC3)cn2)CC1] | 404 |
| 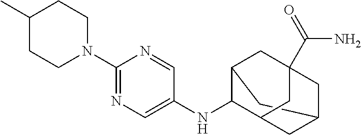 [SMILES: CC1CCN(c2ncc(NC3C4CC5CC3CC(C(N)=O)(C5)C4)cn2)CC1] | 405 |
| 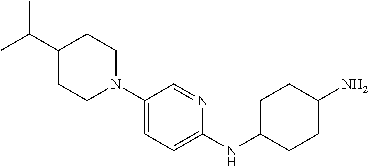 [SMILES: CC(C)C1CCN(c2ccc(NC3CCC(N)CC3)nc2)CC1] | 406 |
| 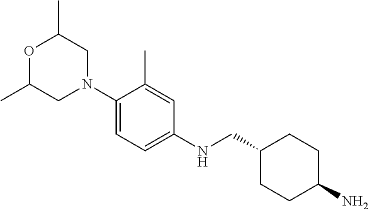 [SMILES: Cc1cc(NC[C@H]2CC[C@H](N)CC2)ccc1N1CC(C)OC(C)C1] | 407 |
| 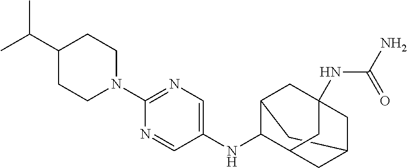 [SMILES: CC(C)C1CCN(c2ncc(NC3C4CC5CC3CC(NC(N)=O)(C5)C4)cn2)CC1] | 408 |
| 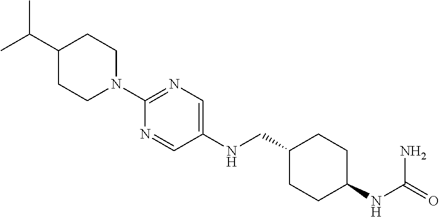 [SMILES: CC(C)C1CCN(c2ncc(NC[C@H]3CC[C@H](NC(N)=O)CC3)cn2)CC1] | 409 |
| 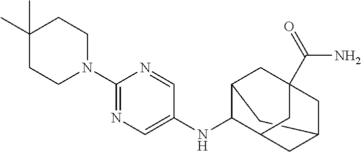 [SMILES: CC1(C)CCN(c2ncc(NC3C4CC5CC3CC(C(N)=O)(C5)C4)cn2)CC1] | 410 |
| 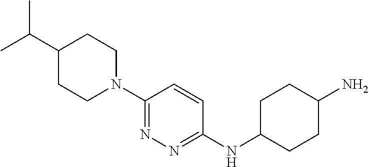 [SMILES: CC(C)C1CCN(c2ccc(NC3CCC(N)CC3)nn2)CC1] | 411 |
| 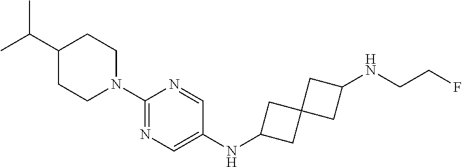 [SMILES: CC(C)C1CCN(c2ncc(NC3CC4(CC(NCCF)C4)C3)cn2)CC1] | 412 |
| 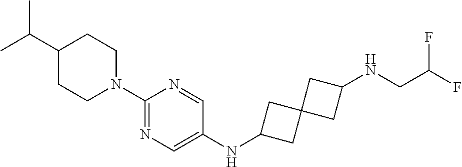 [SMILES: CC(C)C1CCN(c2ncc(NC3CC4(CC(NCC(F)F)C4)C3)cn2)CC1] | 413 |
| 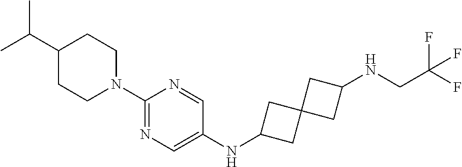 [SMILES: CC(C)C1CCN(c2ncc(NC3CC4(CC(NCC(F)(F)F)C4)C3)cn2)CC1] | 414 |
| 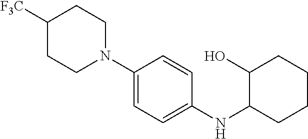 [SMILES: OC1CCCCC1Nc1ccc(N2CCC(C(F)(F)F)CC2)cc1] | 415 |
| 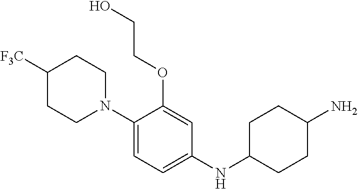 [SMILES: NC1CCC(Nc2ccc(N3CCC(C(F)(F)F)CC3)c(OCCO)c2)CC1] | 416 |
| 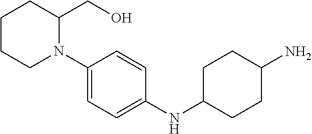 [SMILES: NC1CCC(Nc2ccc(N3CCCCC3CO)cc2)CC1] | 417A/B |
| 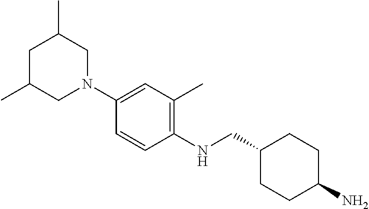 [SMILES: Cc1cc(N2CC(C)CC(C)C2)ccc1NC[C@H]1CC[C@H](N)CC1] | 418 |
| 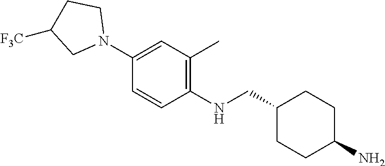 [SMILES: Cc1cc(N2CCC(C(F)(F)F)C2)ccc1NC[C@H]1CC[C@H](N)CC1] | 419 |
| 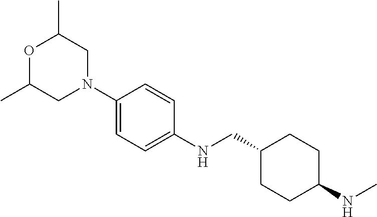 | 420 |
| 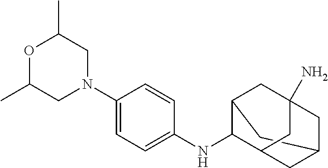 [SMILES: CC1CN(c2ccc(NC3C4CC5CC3CC(N)(C5)C4)cc2)CC(C)O1] | 421A/B |
| 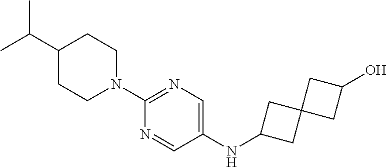 [SMILES: CC(C)C1CCN(c2ncc(NC3CC4(CC(O)C4)C3)cn2)CC1] | 422 |
| 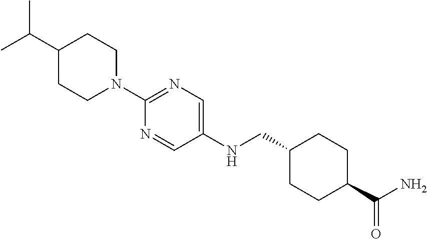 [SMILES: CC(C)C1CCN(c2ncc(NC[C@H]3CC[C@H](C(N)=O)CC3)cn2)CC1] | 423 |
| 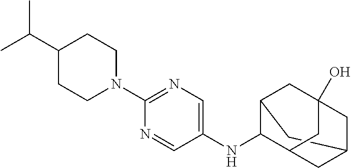 [SMILES: CC(C)C1CCN(c2ncc(NC3C4CC5CC3CC(O)(C5)C4)cn2)CC1] | 424 |
| 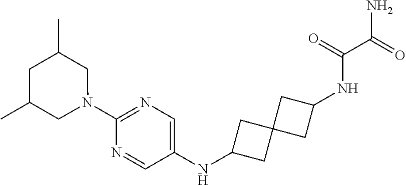 [SMILES: CC1CC(C)CN(c2ncc(NC3CC4(CC(NC(=O)C(N)=O)C4)C3)cn2)C1] | 425 |
| 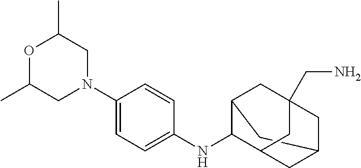 [SMILES: CC1CN(c2ccc(NC3C4CC5CC3CC(CN)(C5)C4)cc2)CC(C)O1] | 426 |
| 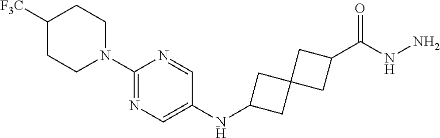 [SMILES: NNC(=O)C1CC2(CC(Nc3cnc(N4CCC(C(F)(F)F)CC4)nc3)C2)C1] | 427 |
| 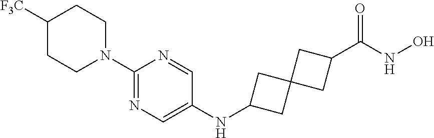 [SMILES: O=C(NO)C1CC2(CC(Nc3cnc(N4CCC(C(F)(F)F)CC4)nc3)C2)C1] | 428 |
| 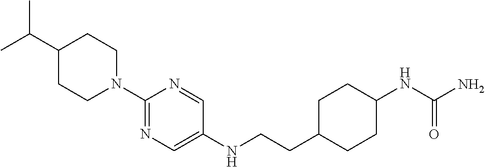 [SMILES: CC(C)C1CCN(c2ncc(NCCC3CCC(NC(N)=O)CC3)cn2)CC1] | 429 |
| 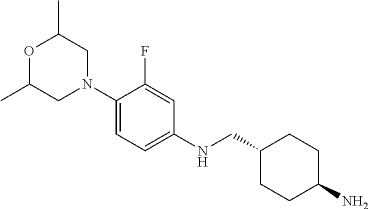 [SMILES: CC1CN(c2ccc(NC[C@H]3CC[C@H](N)CC3)cc2F)CC(C)O1] | 430 |
| 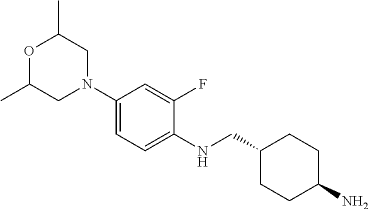 [SMILES: CC1CN(c2ccc(NC[C@H]3CC[C@H](N)CC3)c(F)c2)CC(C)O1] | 431 |
| 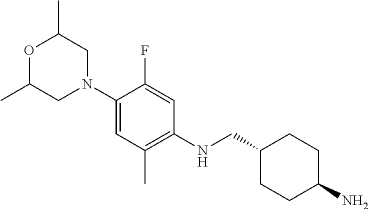 [SMILES: Cc1cc(N2CC(C)OC(C)C2)c(F)cc1NC[C@H]1CC[C@H](N)CC1] | 432 |
| 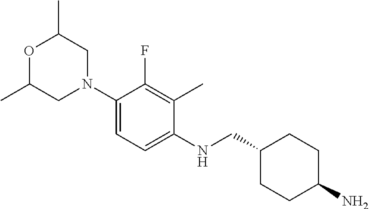 [SMILES: Cc1c(NC[C@H]2CC[C@H](N)CC2)ccc(N2CC(C)OC(C)C2)c1F] | 433 |
| 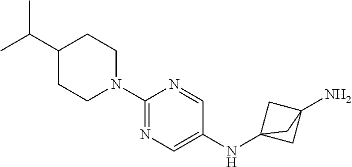 [SMILES: CC(C)C1CCN(c2ncc(NC34CC(N)(C3)C4)cn2)CC1] | 434 |
| 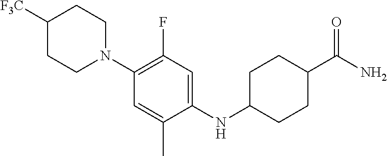 [SMILES: Cc1cc(N2CCC(C(F)(F)F)CC2)c(F)cc1NC1CCC(C(N)=O)CC1] | 435 |
| 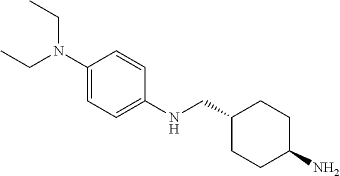 [SMILES: CCN(CC)c1ccc(NC[C@H]2CC[C@H](N)CC2)cc1] | 436 |
| 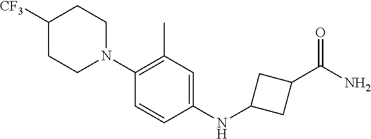 [SMILES: Cc1cc(NC2CC(C(N)=O)C2)ccc1N1CCC(C(F)(F)F)CC1] | 437 |
| 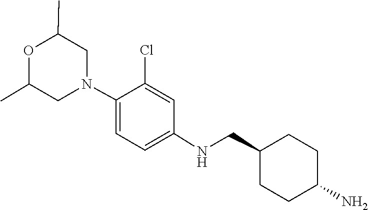 [SMILES: CC1CN(c2ccc(NC[C@H]3CC[C@H](N)CC3)cc2Cl)CC(C)O1] | 438 |
| 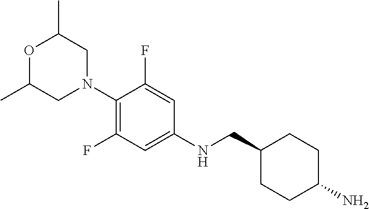 [SMILES: CC1CN(c2c(F)cc(NC[C@H]3CC[C@H](N)CC3)cc2F)CC(C)O1] | 439 |
| 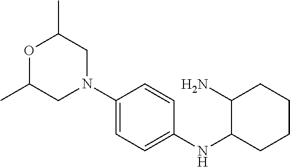 [SMILES: CC1CN(c2ccc(NC3CCCCC3N)cc2)CC(C)O1] | 440 |
| 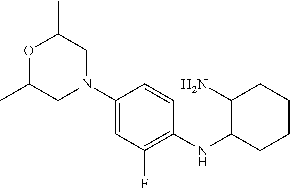 [SMILES: CC1CN(c2ccc(NC3CCCCC3N)c(F)c2)CC(C)O1] | 441 |
| 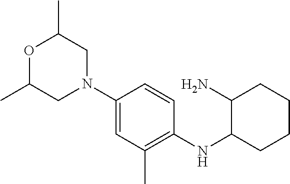 [SMILES: Cc1cc(N2CC(C)OC(C)C2)ccc1NC1CCCCC1N] | 442 |
| 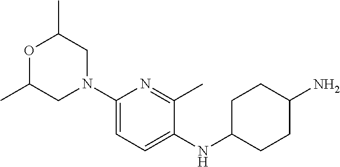 [SMILES: Cc1nc(N2CC(C)OC(C)C2)ccc1NC1CCC(N)CC1] | 443 |
| 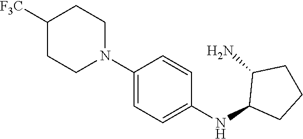 [SMILES: N[C@@H]1CCC[C@H]1Nc1ccc(N2CCC(C(F)(F)F)CC2)cc1] | 444 |
| 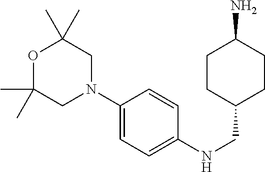 [SMILES: CC1(C)CN(c2ccc(NC[C@H]3CC[C@H](N)CC3)cc2)CC(C)(C)O1] | 445 |
| 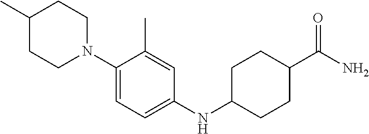 [SMILES: Cc1cc(NC2CCC(C(N)=O)CC2)ccc1N1CCC(C)CC1] | 446A/B |
| 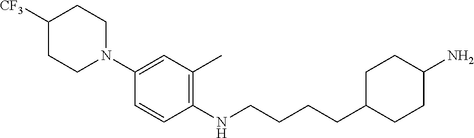 [SMILES: Cc1cc(N2CCC(C(F)(F)F)CC2)ccc1NCCCCC1CCC(N)CC1] | 447 |
| 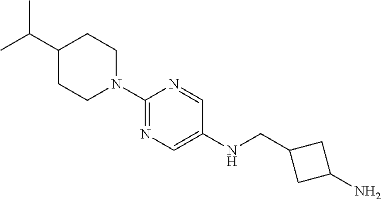 [SMILES: CC(C)C1CCN(c2ncc(NCC3CC(N)C3)cn2)CC1] | 448 |
| 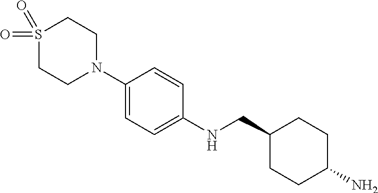 [SMILES: N[C@H]1CC[C@H](CNc2ccc(N3CCS(=O)(=O)CC3)cc2)CC1] | 449 |
| 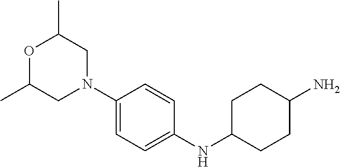 [SMILES: CC1CN(c2ccc(NC3CCC(N)CC3)cc2)CC(C)O1] | 450 |
| 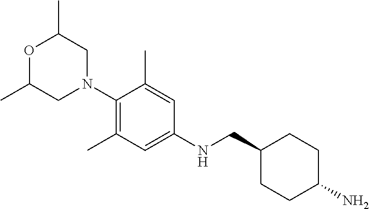 [SMILES: Cc1cc(NC[C@H]2CC[C@H](N)CC2)cc(C)c1N1CC(C)OC(C)C1] | 451 |
| 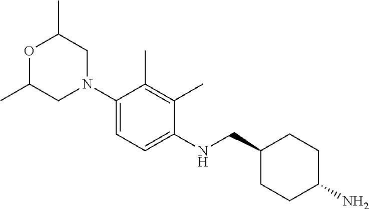 [SMILES: Cc1c(NC[C@H]2CC[C@H](N)CC2)ccc(N2CC(C)OC(C)C2)c1C] | 452 |
| 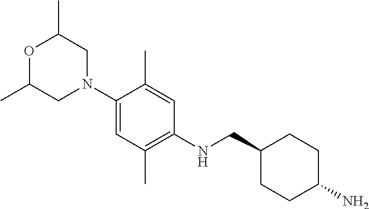 [SMILES: Cc1cc(N2CC(C)OC(C)C2)c(C)cc1NC[C@H]1CC[C@H](N)CC1] | 453 |
| 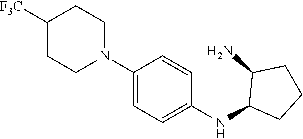 [SMILES: N[C@H]1CCC[C@H]1Nc1ccc(N2CCC(C(F)(F)F)CC2)cc1] | 454 |
| 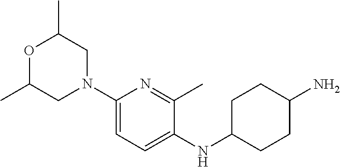 [SMILES: Cc1nc(N2CC(C)OC(C)C2)ccc1NC1CCC(N)CC1] | 455 |
| 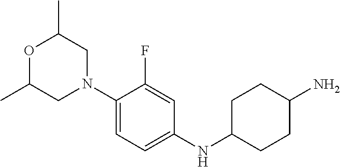 [SMILES: CC1CN(c2ccc(NC3CCC(N)CC3)cc2F)CC(C)O1] | 456 |
| 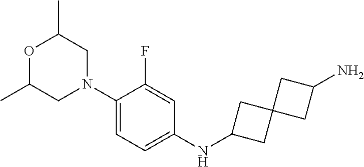 [SMILES: CC1CN(c2ccc(NC3CC4(CC(N)C4)C3)cc2F)CC(C)O1] | 457 |
| 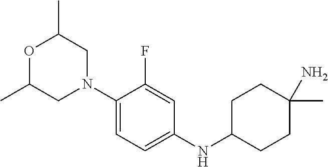 | 458 |
| 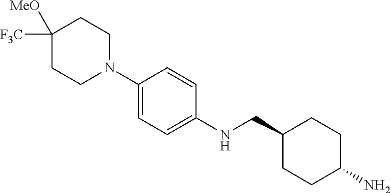 [SMILES: COC1(C(F)(F)F)CCN(c2ccc(NC[C@H]3CC[C@H](N)CC3)cc2)CC1] | 459 |
| 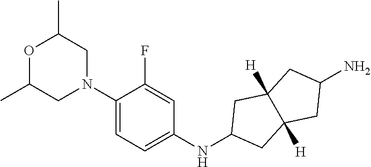 [SMILES: CC1CN(c2ccc(NC3C[C@H]4CC(N)C[C@H]4C3)cc2F)CC(C)O1] | 460 |
| 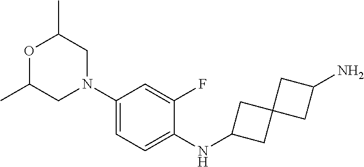 [SMILES: CC1CN(c2ccc(NC3CC4(CC(N)C4)C3)c(F)c2)CC(C)O1] | 461 |
| 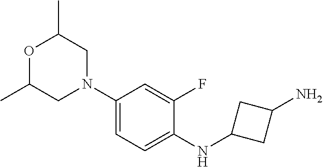 [SMILES: CC1CN(c2ccc(NC3CC(N)C3)c(F)c2)CC(C)O1] | 462 |
| 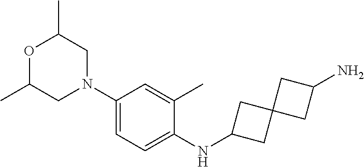 [SMILES: Cc1cc(N2CC(C)OC(C)C2)ccc1NC1CC2(CC(N)C2)C1] | 463 |
| 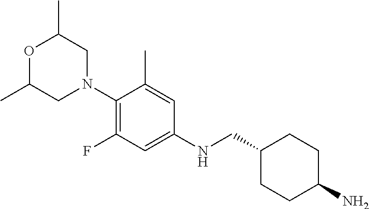 [SMILES: Cc1cc(NC[C@H]2CC[C@H](N)CC2)cc(F)c1N1CC(C)OC(C)C1] | 464 |
| 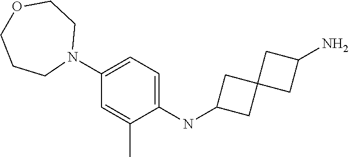 | 465 |
| 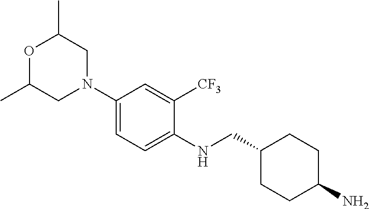 [SMILES: CC1CN(c2ccc(NC[C@H]3CC[C@H](N)CC3)c(C(F)(F)F)c2)CC(C)O1] | 466 |
| 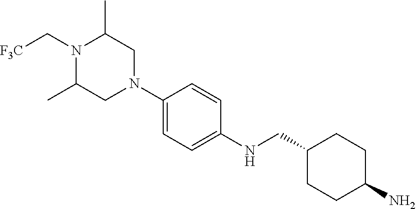 [SMILES: CC1CN(c2ccc(NC[C@H]3CC[C@H](N)CC3)cc2)CC(C)N1CC(F)(F)F] | 467 |
| 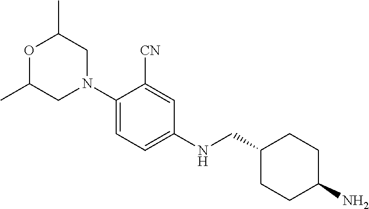 [SMILES: CC1CN(c2ccc(NC[C@H]3CC[C@H](N)CC3)cc2C#N)CC(C)O1] | 468 |
| 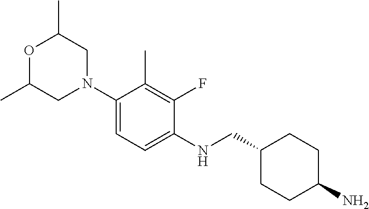 [SMILES: Cc1c(N2CC(C)OC(C)C2)ccc(NC[C@H]2CC[C@H](N)CC2)c1F] | 469 |
| 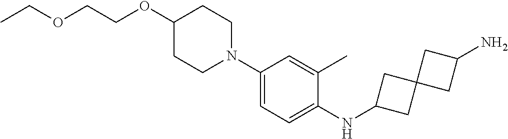 [SMILES: CCOCCOC1CCN(c2ccc(NC3CC4(CC(N)C4)C3)c(C)c2)CC1] | 470 |
| 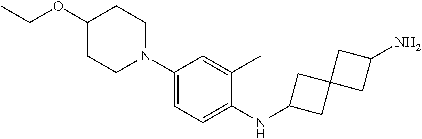 [SMILES: CCOC1CCN(c2ccc(NC3CC4(CC(N)C4)C3)c(C)c2)CC1] | 471 |
| 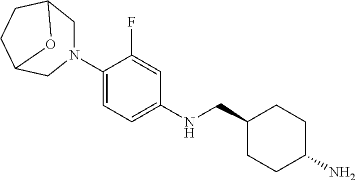 [SMILES: N[C@H]1CC[C@H](CNc2ccc(N3CC4CCC(C3)O4)c(F)c2)CC1] | 472 |
| 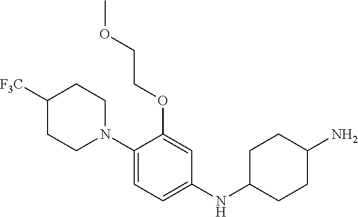 [SMILES: COCCOc1cc(NC2CCC(N)CC2)ccc1N1CCC(C(F)(F)F)CC1] | 473 |
| 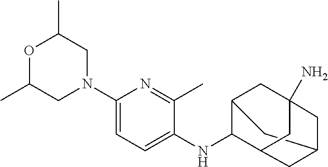 [SMILES: Cc1nc(N2CC(C)OC(C)C2)ccc1NC1C2CC3CC1CC(N)(C3)C2] | 474 |
| 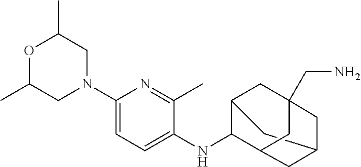 [SMILES: Cc1nc(N2CC(C)OC(C)C2)ccc1NC1C2CC3CC1CC(CN)(C3)C2] | 475A/B |
| 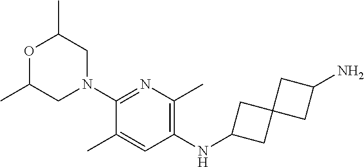 [SMILES: Cc1cc(NC2CC3(CC(N)C3)C2)c(C)nc1N1CC(C)OC(C)C1] | 476 |
| 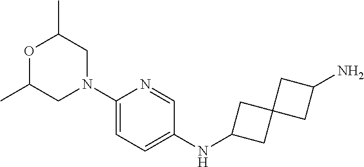 [SMILES: CC1CN(c2ccc(NC3CC4(CC(N)C4)C3)cn2)CC(C)O1] | 477 |
| 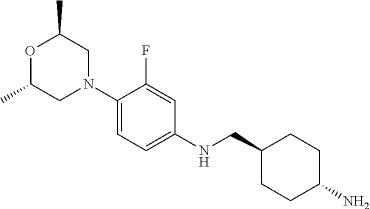 [SMILES: C[C@H]1CN(c2ccc(NC[C@H]3CC[C@H](N)CC3)cc2F)C[C@H](C)O1] | 478 |
| 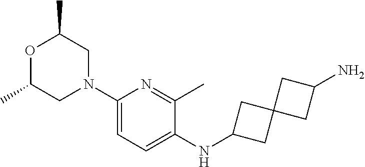 [SMILES: Cc1nc(N2C[C@H](C)O[C@@H](C)C2)ccc1NC1CC2(CC(N)C2)C1] | 479 |
| 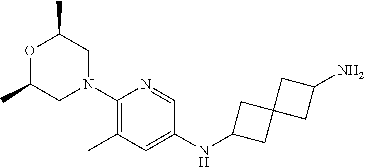 [SMILES: Cc1cc(NC2CC3(CC(N)C3)C2)cnc1N1C[C@@H](C)O[C@@H](C)C1] | 480 |
| 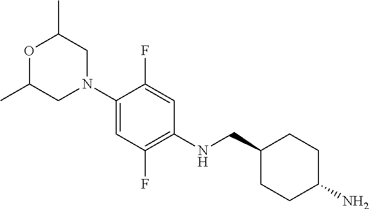 [SMILES: CC1CN(c2cc(F)c(NC[C@H]3CC[C@H](N)CC3)cc2F)CC(C)O1] | 481 |
| 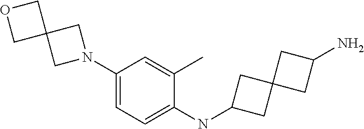 | 482 |
| 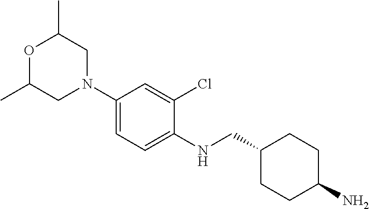 [SMILES: CC1CN(c2ccc(NC[C@H]3CC[C@H](N)CC3)c(Cl)c2)CC(C)O1] | 483 |
| 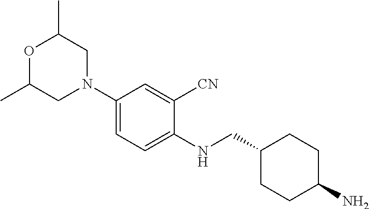 [SMILES: CC1CN(c2ccc(NC[C@H]3CC[C@H](N)CC3)c(C#N)c2)CC(C)O1] | 484 |
| 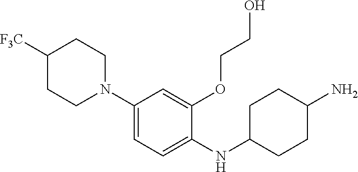 [SMILES: NC1CCC(Nc2ccc(N3CCC(C(F)(F)F)CC3)cc2OCCO)CC1] | 485 |
| 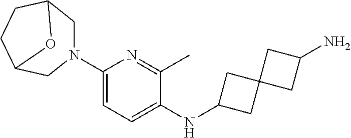 [SMILES: Cc1nc(N2CC3CCC(C2)O3)ccc1NC1CC2(CC(N)C2)C1] | 486 |
| 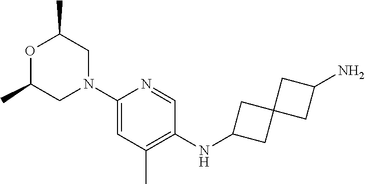 [SMILES: Cc1cc(N2C[C@@H](C)O[C@@H](C)C2)ncc1NC1CC2(CC(N)C2)C1] | 487 |
| 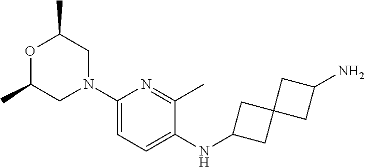 [SMILES: Cc1nc(N2C[C@@H](C)O[C@@H](C)C2)ccc1NC1CC2(CC(N)C2)C1] | 488A/B |
| 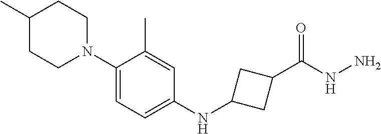 [SMILES: Cc1cc(NC2CC(C(=O)NN)C2)ccc1N1CCC(C)CC1] | 489 |
| 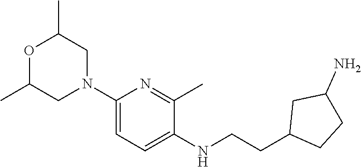 [SMILES: Cc1nc(N2CC(C)OC(C)C2)ccc1NCCC1CCC(N)C1] | 490 |
| 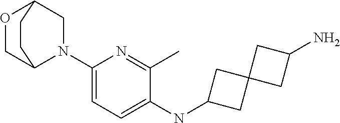 | 491 |
| 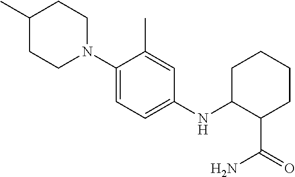 [SMILES: Cc1cc(NC2CCCCC2C(N)=O)ccc1N1CCC(C)CC1] | 492 |
| 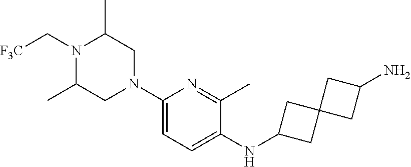 [SMILES: Cc1nc(N2CC(C)N(CC(F)(F)F)C(C)C2)ccc1NC1CC2(CC(N)C2)C1] | 493 |
| 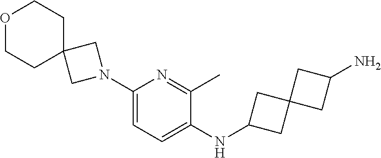 [SMILES: Cc1nc(N2CC3(CCOCC3)C2)ccc1NC1CC2(CC(N)C2)C1] | 494 |
| 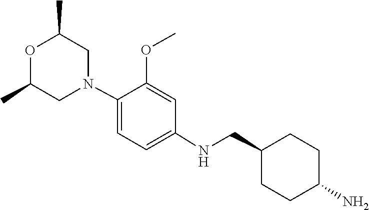 [SMILES: COc1cc(NC[C@H]2CC[C@H](N)CC2)ccc1N1C[C@@H](C)O[C@@H](C)C1] | 495 |
| 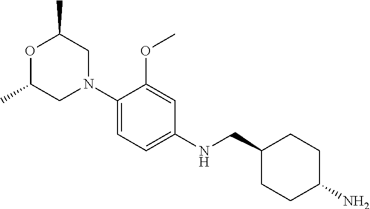 [SMILES: COc1cc(NC[C@H]2CC[C@H](N)CC2)ccc1N1C[C@H](C)O[C@@H](C)C1] | 496 |
| 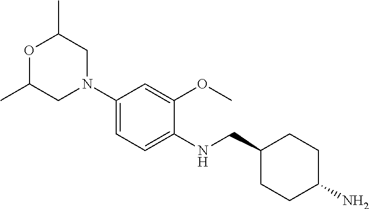 [SMILES: COc1cc(N2CC(C)OC(C)C2)ccc1NC[C@H]1CC[C@H](N)CC1] | 497 |
| 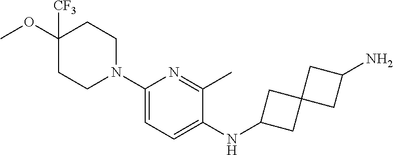 [SMILES: COC1(C(F)(F)F)CCN(c2ccc(NC3CC4(CC(N)C4)C3)c(C)n2)CC1] | 498 |
| 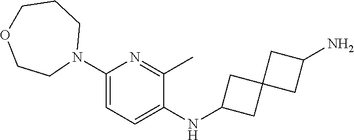 [SMILES: Cc1nc(N2CCCOCC2)ccc1NC1CC2(CC(N)C2)C1] | 499 |
| 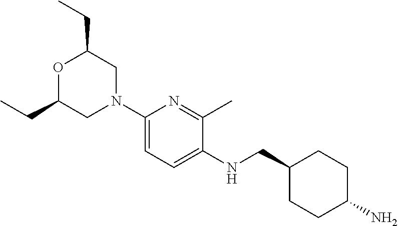 [SMILES: CC[C@@H]1CN(c2ccc(NC[C@H]3CC[C@H](N)CC3)c(C)n2)C[C@H](CC)O1] | 500 |
| 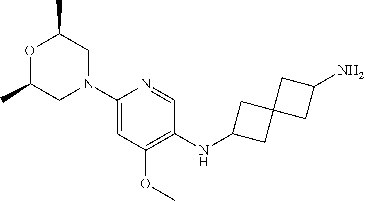 [SMILES: COc1cc(N2C[C@@H](C)O[C@@H](C)C2)ncc1NC1CC2(CC(N)C2)C1] | 501 |
| 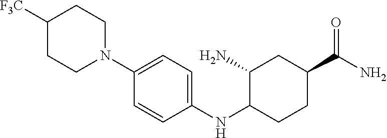 [SMILES: NC(=O)[C@H]1CCC(Nc2ccc(N3CCC(C(F)(F)F)CC3)cc2)[C@H](N)C1] | 502 |
| 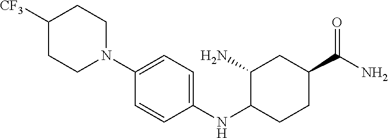 [SMILES: NC(=O)[C@H]1CCC(Nc2ccc(N3CCC(C(F)(F)F)CC3)cc2)[C@H](N)C1] | 503 |
| 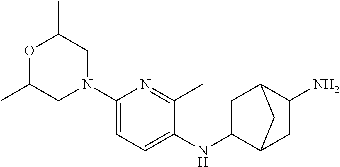 [SMILES: Cc1nc(N2CC(C)OC(C)C2)ccc1NC1CC2CC1CC2N] | 504 |
| 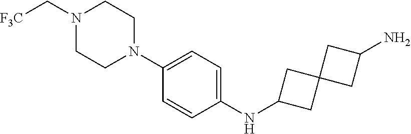 [SMILES: NC1CC2(C1)CC(Nc1ccc(N3CCN(CC(F)(F)F)CC3)cc1)C2] | 505 |
| 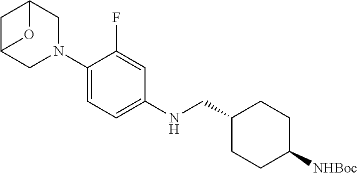 [SMILES: CC(C)(C)OC(=O)N[C@H]1CC[C@H](CNc2ccc(N3CC4CC(C3)O4)c(F)c2)CC1] | 506 |
| 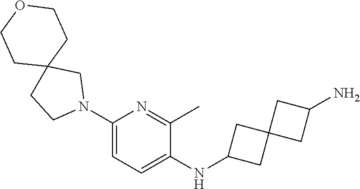 [SMILES: Cc1nc(N2CCC3(CCOCC3)C2)ccc1NC1CC2(CC(N)C2)C1] | 507 |
| 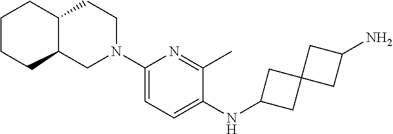 [SMILES: Cc1nc(N2CC[C@H]3CCCC[C@@H]3C2)ccc1NC1CC2(CC(N)C2)C1] | 508 |
| 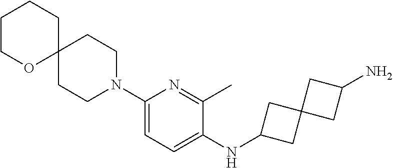 [SMILES: Cc1nc(N2CCC3(CCCCO3)CC2)ccc1NC1CC2(CC(N)C2)C1] | 509 |
| 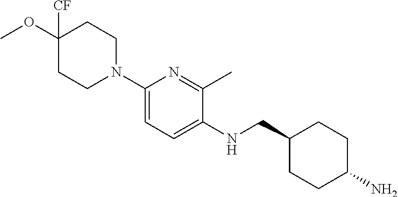 | 510 |
| 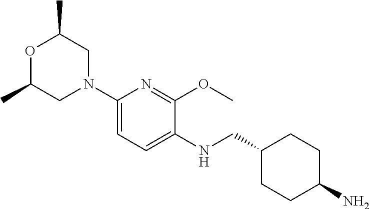 [SMILES: COc1nc(N2C[C@@H](C)O[C@@H](C)C2)ccc1NC[C@H]1CC[C@H](N)CC1] | 511 |
| 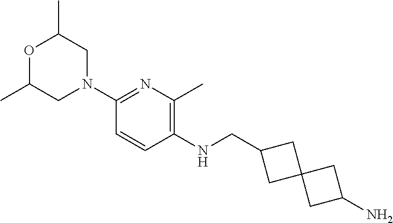 [SMILES: Cc1nc(N2CC(C)OC(C)C2)ccc1NCC1CC2(CC(N)C2)C1] | 512 |
| 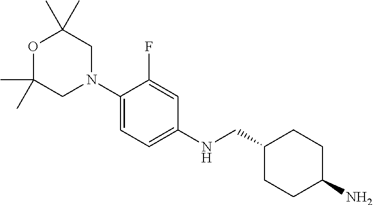 [SMILES: CC1(C)CN(c2ccc(NC[C@H]3CC[C@H](N)CC3)cc2F)CC(C)(C)O1] | 513 |
| 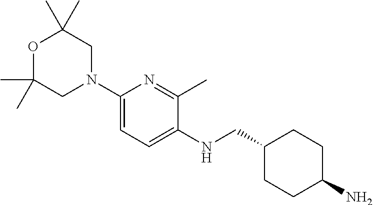 [SMILES: Cc1nc(N2CC(C)(C)OC(C)(C)C2)ccc1NC[C@H]1CC[C@H](N)CC1] | 514 |
| 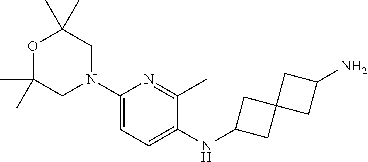 [SMILES: Cc1nc(N2CC(C)(C)OC(C)(C)C2)ccc1NC1CC2(CC(N)C2)C1] | 515 |
| 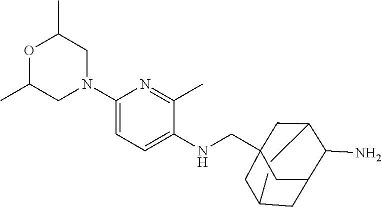 [SMILES: Cc1nc(N2CC(C)OC(C)C2)ccc1NCC12CC(C)CC(C1)C(N)C(C)C2] | 516A/B |
| 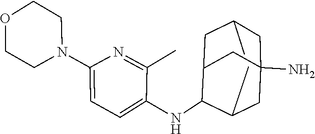 [SMILES: Cc1nc(N2CCOCC2)ccc1NC1C(C)CC2(N)CC(C)CC1C2] | 517 |
| 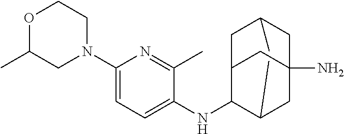 [SMILES: Cc1nc(N2CCOC(C)C2)ccc1NC1C(C)CC2(N)CC(C)CC1C2] | 518 |
| 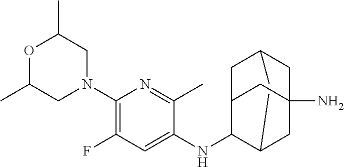 [SMILES: Cc1nc(N2CC(C)OC(C)C2)c(F)cc1NC1C(C)CC2(N)CC(C)CC1C2] | 519 |
| 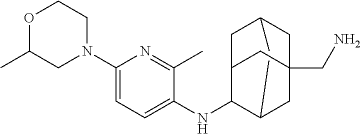 [SMILES: Cc1nc(N2CCOC(C)C2)ccc1NC1C(C)CC2(CN)CC(C)CC1C2] | 520 |
| 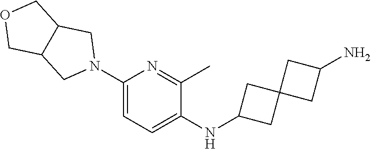 [SMILES: Cc1nc(N2CC3COCC3C2)ccc1NC1CC2(CC(N)C2)C1] | 521 |
| 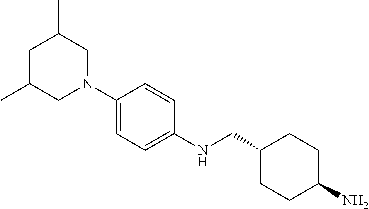 [SMILES: CC1CC(C)CN(c2ccc(NC[C@H]3CC[C@H](N)CC3)cc2)C1] | 522 |
| 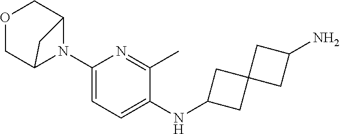 [SMILES: Cc1nc(N2C3COCC2C3)ccc1NC1CC2(CC(N)C2)C1] | 523 |
| 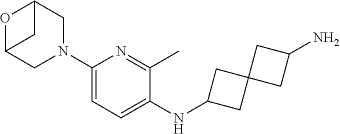 [SMILES: Cc1nc(N2CC3CC(C2)O3)ccc1NC1CC2(CC(N)C2)C1] | 524 |
| 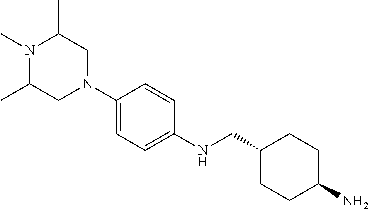 [SMILES: CC1CN(c2ccc(NC[C@H]3CC[C@H](N)CC3)cc2)CC(C)N1C] | 525 |
| 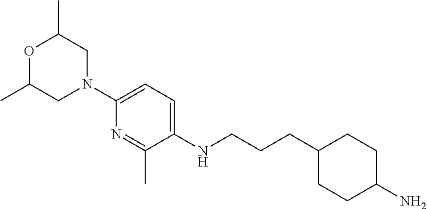 [SMILES: Cc1nc(N2CC(C)OC(C)C2)ccc1NCCCC1CCC(N)CC1] | 526 |
| 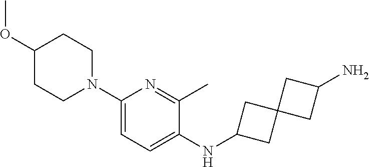 [SMILES: COC1CCN(c2ccc(NC3CC4(CC(N)C4)C3)c(C)n2)CC1] | 527 |
| 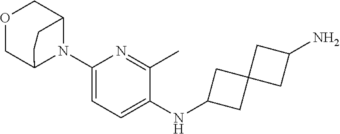 [SMILES: Cc1nc(N2C3CCC2COC3)ccc1NC1CC2(CC(N)C2)C1] | 528 |
| 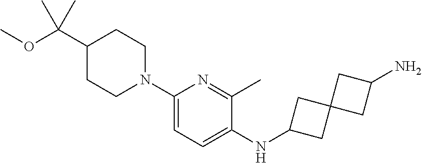 [SMILES: COC(C)(C)C1CCN(c2ccc(NC3CC4(CC(N)C4)C3)c(C)n2)CC1] | 529 |
| 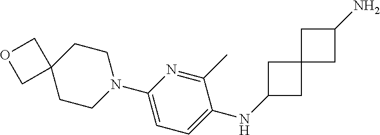 [SMILES: Cc1nc(N2CCC3(CC2)COC3)ccc1NC1CC2(CC(N)C2)C1] | 530 |
| 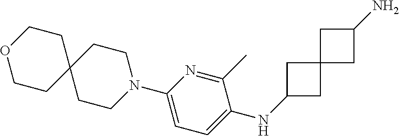 [SMILES: Cc1nc(N2CCC3(CCOCC3)CC2)ccc1NC1CC2(CC(N)C2)C1] | 531 |
| 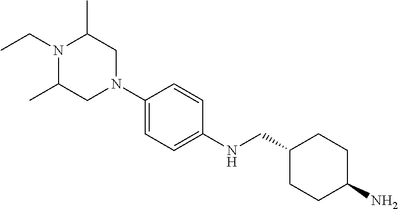 [SMILES: CCN1C(C)CN(c2ccc(NC[C@H]3CC[C@H](N)CC3)cc2)CC1C] | 532 |
| 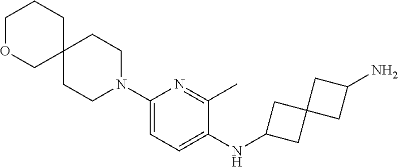 [SMILES: Cc1nc(N2CCC3(CCCOC3)CC2)ccc1NC1CC2(CC(N)C2)C1] | 533 |
| 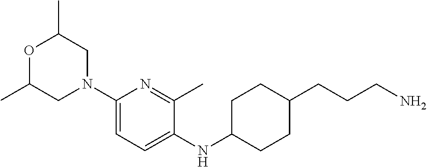 [SMILES: Cc1nc(N2CC(C)OC(C)C2)ccc1NC1CCC(CCCN)CC1] | 534 |
| 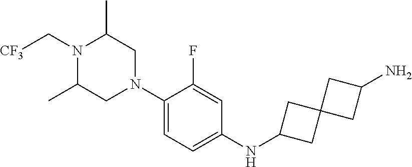 [SMILES: CC1CN(c2ccc(NC3CC4(CC(N)C4)C3)cc2F)CC(C)N1CC(F)(F)F] | 535 |
| 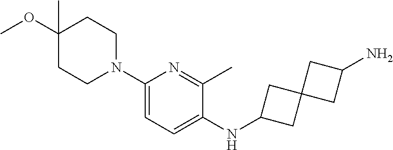 [SMILES: COC1(C)CCN(c2ccc(NC3CC4(CC(N)C4)C3)c(C)n2)CC1] | 536 |
| 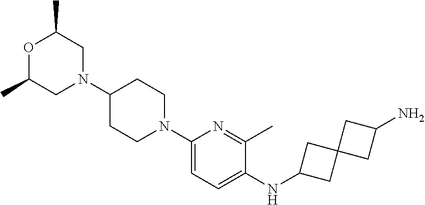 [SMILES: Cc1nc(N2CCC(N3C[C@@H](C)O[C@@H](C)C3)CC2)ccc1NC1CC2(CC(N)C2)C1] | 537 |
| 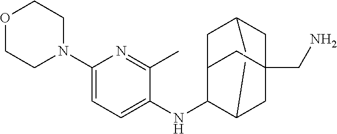 [SMILES: Cc1nc(N2CCOCC2)ccc1NC1C(C)CC2(CN)CC(C)CC1C2] | 538A/B |
| 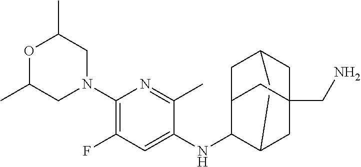 [SMILES: Cc1nc(N2CC(C)OC(C)C2)c(F)cc1NC1C(C)CC2(CN)CC(C)CC1C2] | 539A/B |
| 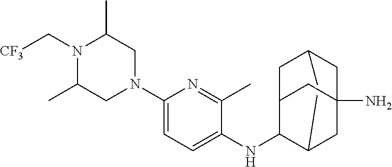 [SMILES: Cc1nc(N2CC(C)N(CC(F)(F)F)C(C)C2)ccc1NC1C(C)CC2(N)CC(C)CC1C2] | 540 |
| 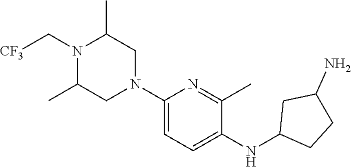 [SMILES: Cc1nc(N2CC(C)N(CC(F)(F)F)C(C)C2)ccc1NC1CCC(N)C1] | 541 |
| 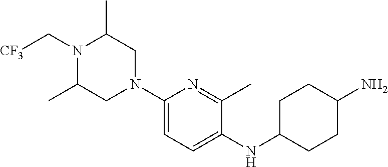 [SMILES: Cc1nc(N2CC(C)N(CC(F)(F)F)C(C)C2)ccc1NC1CCC(N)CC1] | 542 |
| 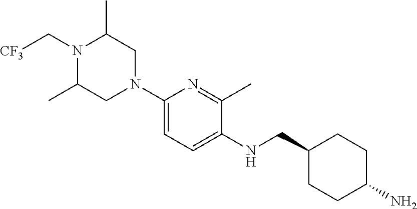 [SMILES: Cc1nc(N2CC(C)N(CC(F)(F)F)C(C)C2)ccc1NC[C@H]1CC[C@H](N)CC1] | 543 |
| 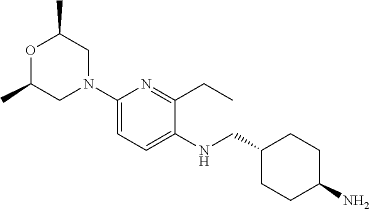 [SMILES: CCc1nc(N2C[C@@H](C)O[C@@H](C)C2)ccc1NC[C@H]1CC[C@H](N)CC1] | 544 |
| 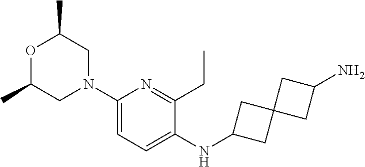 [SMILES: CCc1nc(N2C[C@@H](C)O[C@@H](C)C2)ccc1NC1CC2(CC(N)C2)C1] | 545 |
| 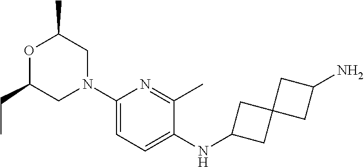 [SMILES: CC[C@@H]1CN(c2ccc(NC3CC4(CC(N)C4)C3)c(C)n2)C[C@H](C)O1] | 546 |
| 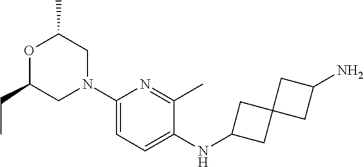 [SMILES: CC[C@@H]1CN(c2ccc(NC3CC4(CC(N)C4)C3)c(C)n2)C[C@@H](C)O1] | 547 |
| 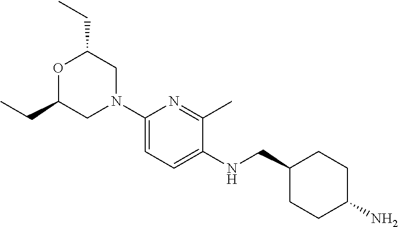 [SMILES: CC[C@@H]1CN(c2ccc(NC[C@H]3CC[C@H](N)CC3)c(C)n2)C[C@@H](CC)O1] | 548 |
| 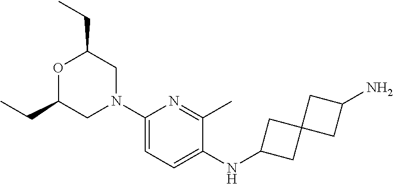 [SMILES: CC[C@@H]1CN(c2ccc(NC3CC4(CC(N)C4)C3)c(C)n2)C[C@H](CC)O1] | 549 |
| 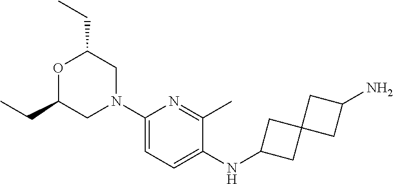 [SMILES: CC[C@@H]1CN(c2ccc(NC3CC4(CC(N)C4)C3)c(C)n2)C[C@@H](CC)O1] | 550 |
| 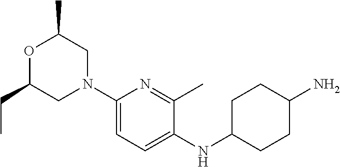 [SMILES: CC[C@@H]1CN(c2ccc(NC3CCC(N)CC3)c(C)n2)C[C@H](C)O1] | 551 |
| 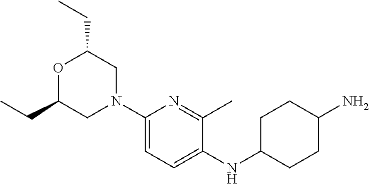 [SMILES: CC[C@@H]1CN(c2ccc(NC3CCC(N)CC3)c(C)n2)C[C@@H](CC)O1] | 552 |
| 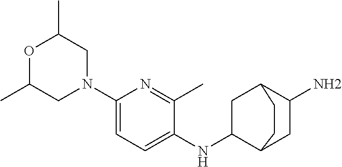 [SMILES: Cc1nc(N2CC(C)OC(C)C2)ccc1NC1CC2CCC1CC2N] | 553 |
| 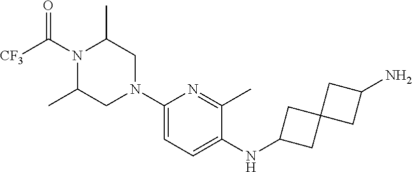 [SMILES: Cc1nc(N2CC(C)N(C(=O)C(F)(F)F)C(C)C2)ccc1NC1CC2(CC(N)C2)C1] | 554 |
| 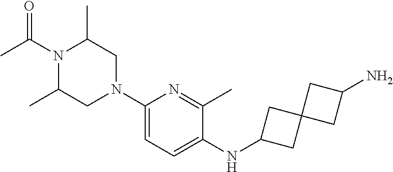 [SMILES: CC(=O)N1C(C)CN(c2ccc(NC3CC4(CC(N)C4)C3)c(C)n2)CC1C] | 555 |
| 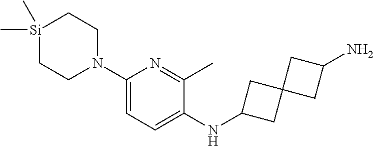 [SMILES: Cc1nc(N2CC[Si](C)(C)CC2)ccc1NC1CC2(CC(N)C2)C1] | 556 |
| 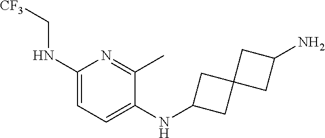 [SMILES: Cc1nc(NCC(F)(F)F)ccc1NC1CC2(CC(N)C2)C1] | 557 |
| 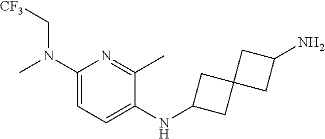 [SMILES: Cc1nc(N(C)CC(F)(F)F)ccc1NC1CC2(CC(N)C2)C1] | 558 |
| 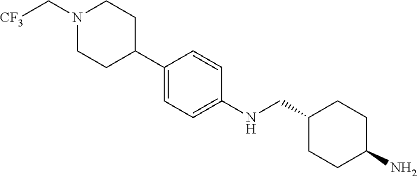 [SMILES: N[C@H]1CC[C@H](CNc2ccc(C3CCN(CC(F)(F)F)CC3)cc2)CC1] | 559 |
| 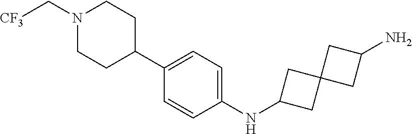 [SMILES: NC1CC2(C1)CC(Nc1ccc(C3CCN(CC(F)(F)F)CC3)cc1)C2] | 560 |
| 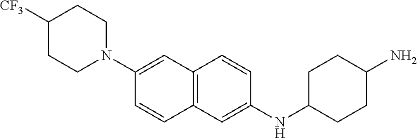 [SMILES: NC1CCC(Nc2ccc3cc(N4CCC(C(F)(F)F)CC4)ccc3c2)CC1] | 561 |
| 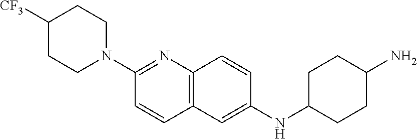 [SMILES: NC1CCC(Nc2ccc3nc(N4CCC(C(F)(F)F)CC4)ccc3c2)CC1] | 562 |
| 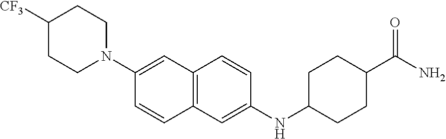 [SMILES: NC(=O)C1CCC(Nc2ccc3cc(N4CCC(C(F)(F)F)CC4)ccc3c2)CC1] | 563 |
| 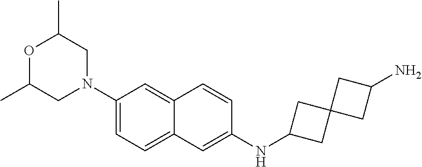 [SMILES: CC1CN(c2ccc3cc(NC4CC5(CC(N)C5)C4)ccc3c2)CC(C)O1] | 564 |
| 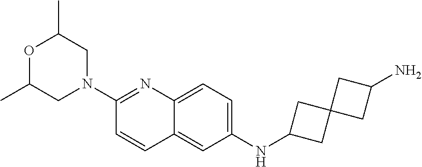 [SMILES: CC1CN(c2ccc3cc(NC4CC5(CC(N)C5)C4)ccc3n2)CC(C)O1] | 565 |
| 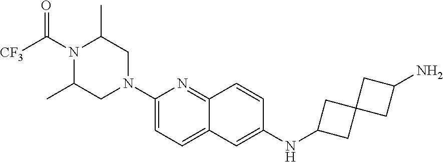 [SMILES: CC1CN(c2ccc3cc(NC4CC5(CC(N)C5)C4)ccc3n2)CC(C)N1C(=O)C(F)(F)F] | 566 |
| 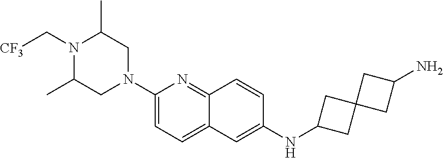 [SMILES: CC1CN(c2ccc3cc(NC4CC5(CC(N)C5)C4)ccc3n2)CC(C)N1CC(F)(F)F] | 567 |
| 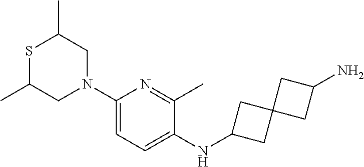 [SMILES: Cc1nc(N2CC(C)SC(C)C2)ccc1NC1CC2(CC(N)C2)C1] | 568 |
| 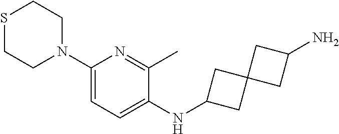 [SMILES: Cc1nc(N2CCSCC2)ccc1NC1CC2(CC(N)C2)C1] | 569 |
| 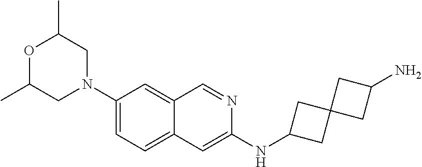 [SMILES: CC1CN(c2ccc3cc(NC4CC5(CC(N)C5)C4)ncc3c2)CC(C)O1] | 570 |
| 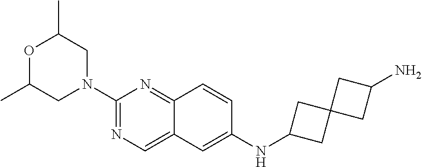 [SMILES: CC1CN(c2ncc3cc(NC4CC5(CC(N)C5)C4)ccc3n2)CC(C)O1] | 571 |
| 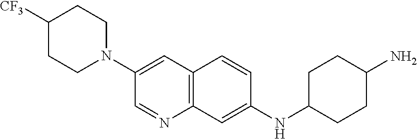 [SMILES: NC1CCC(Nc2ccc3cc(N4CCC(C(F)(F)F)CC4)cnc3c2)CC1] | 572 |
| 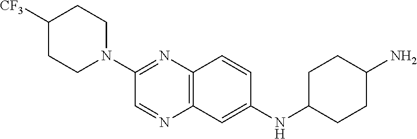 [SMILES: NC1CCC(Nc2ccc3nc(N4CCC(C(F)(F)F)CC4)cnc3c2)CC1] | 573 |
| 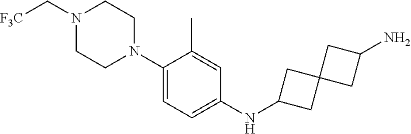 [SMILES: Cc1cc(NC2CC3(CC(N)C3)C2)ccc1N1CCN(CC(F)(F)F)CC1] | 574 |
| 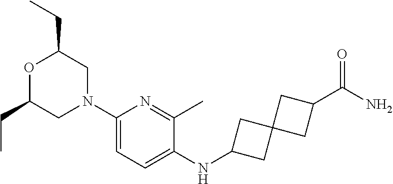 [SMILES: CC[C@@H]1CN(c2ccc(NC3CC4(C3)CC(C(N)=O)C4)c(C)n2)C[C@H](CC)O1] | 575 |
| 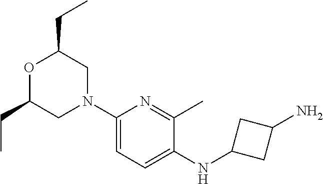 [SMILES: CC[C@@H]1CN(c2ccc(NC3CC(N)C3)c(C)n2)C[C@H](CC)O1] | 576 |
| 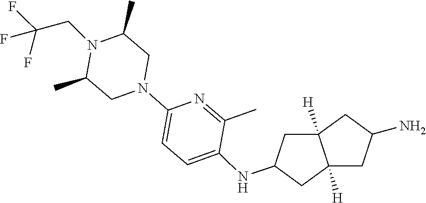 [SMILES: Cc1nc(N2C[C@@H](C)N(CC(F)(F)F)[C@@H](C)C2)ccc1NC1C[C@H]2CC(N)C[C@H]2C1] | 577 |
| 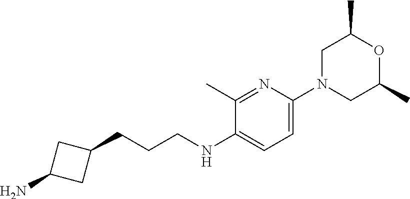 [SMILES: Cc1nc(N2C[C@@H](C)O[C@@H](C)C2)ccc1NCCC[C@H]1C[C@@H](N)C1] | 578 |
| 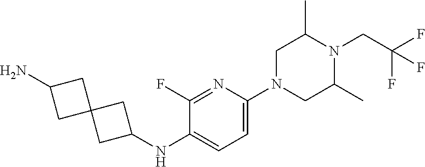 [SMILES: CC1CN(c2ccc(NC3CC4(CC(N)C4)C3)c(F)n2)CC(C)N1CC(F)(F)F] | 579 |
| 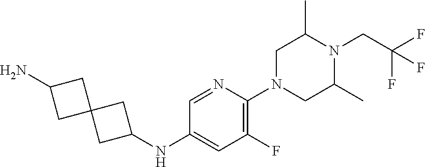 [SMILES: CC1CN(c2ncc(NC3CC4(CC(N)C4)C3)cc2F)CC(C)N1CC(F)(F)F] | 580 |
| 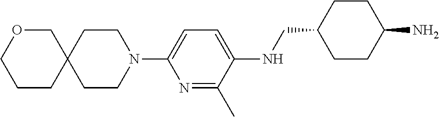 [SMILES: Cc1nc(N2CCC3(CCCOC3)CC2)ccc1NC[C@H]1CC[C@H](N)CC1] | 581 |
| 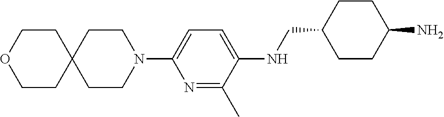 [SMILES: Cc1nc(N2CCC3(CCOCC3)CC2)ccc1NC[C@H]1CC[C@H](N)CC1] | 582 |
| 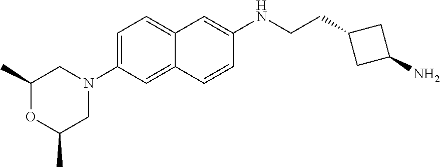 [SMILES: C[C@@H]1CN(c2ccc3cc(NCC[C@H]4C[C@H](N)C4)ccc3c2)C[C@H](C)O1] | 583 |
| 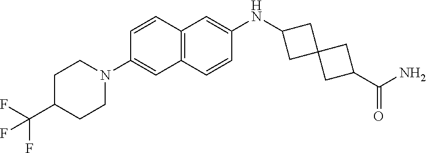 [SMILES: NC(=O)C1CC2(CC(Nc3ccc4cc(N5CCC(C(F)(F)F)CC5)ccc4c3)C2)C1] | 584 |
| 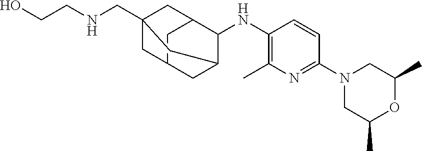 [SMILES: Cc1nc(N2C[C@@H](C)O[C@@H](C)C2)ccc1NC1C2CC3CC1CC(CNCCO)(C3)C2] | 585 |
| 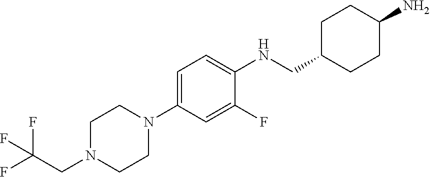 [SMILES: N[C@H]1CC[C@H](CNc2ccc(N3CCN(CC(F)(F)F)CC3)cc2F)CC1] | 586 |
| 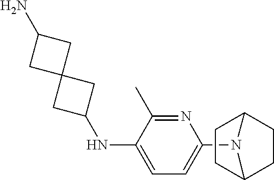 [SMILES: Cc1nc(N2C3CCC2CC3)ccc1NC1CC2(CC(N)C2)C1] | 587 |
| 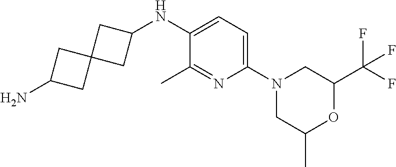 [SMILES: Cc1nc(N2CC(C)OC(C(F)(F)F)C2)ccc1NC1CC2(CC(N)C2)C1] | 588 |
| 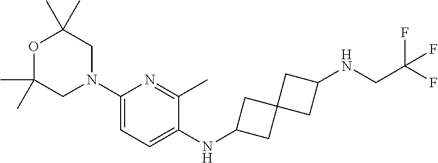 [SMILES: Cc1nc(N2CC(C)(C)OC(C)(C)C2)ccc1NC1CC2(CC(NCC(F)(F)F)C2)C1] | 589 |
| 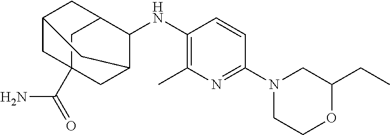 [SMILES: CCC1CN(c2ccc(NC3C4CC5CC3CC(C(N)=O)(C5)C4)c(C)n2)CCO1] | 590 |
| 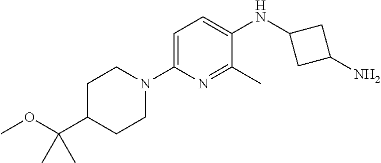 [SMILES: COC(C)(C)C1CCN(c2ccc(NC3CC(N)C3)c(C)n2)CC1] | 591 |
| 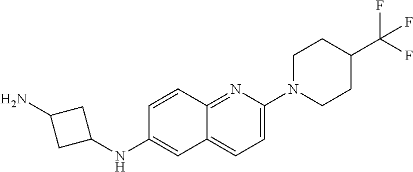 [SMILES: NC1CC(Nc2ccc3nc(N4CCC(C(F)(F)F)CC4)ccc3c2)C1] | 592 |
| 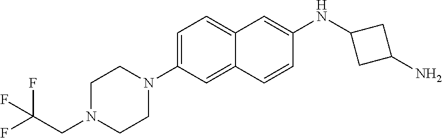 [SMILES: NC1CC(Nc2ccc3cc(N4CCN(CC(F)(F)F)CC4)ccc3c2)C1] | 593 |
| 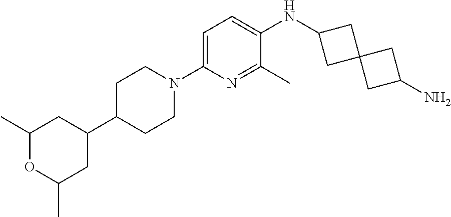 [SMILES: Cc1nc(N2CCC(C3CC(C)OC(C)C3)CC2)ccc1NC1CC2(CC(N)C2)C1] | 594 |
| 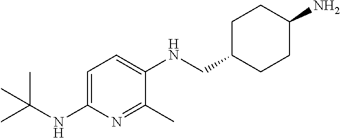 [SMILES: Cc1nc(NC(C)(C)C)ccc1NC[C@H]1CC[C@H](N)CC1] | 595 |
| 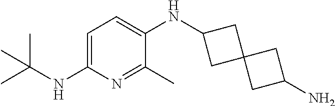 [SMILES: Cc1nc(NC(C)(C)C)ccc1NC1CC2(CC(N)C2)C1] | 596 |
| 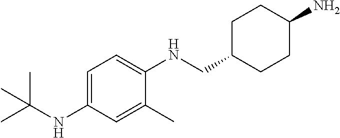 [SMILES: Cc1cc(NC(C)(C)C)ccc1NC[C@H]1CC[C@H](N)CC1] | 597 |
| 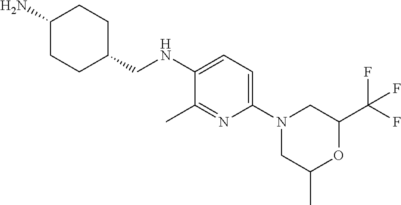 [SMILES: Cc1nc(N2CC(C)OC(C(F)(F)F)C2)ccc1NC[C@H]1CC[C@@H](N)CC1] | 598 |
| 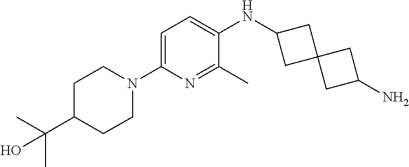 [SMILES: Cc1nc(N2CCC(C(C)(C)O)CC2)ccc1NC1CC2(CC(N)C2)C1] | 599 |
| 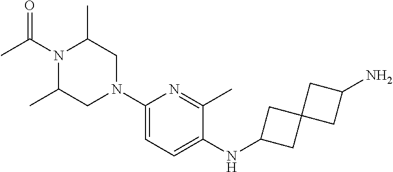 [SMILES: CC(=O)N1C(C)CN(c2ccc(NC3CC4(CC(N)C4)C3)c(C)n2)CC1C] | 600 |
| 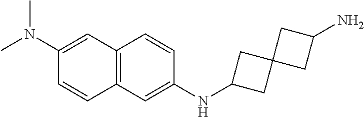 [SMILES: CN(C)c1ccc2cc(NC3CC4(CC(N)C4)C3)ccc2c1] | 601 |
| 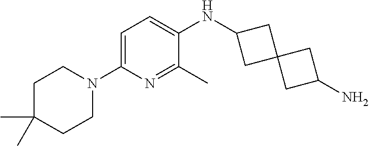 [SMILES: Cc1nc(N2CCC(C)(C)CC2)ccc1NC1CC2(CC(N)C2)C1] | 602 |
| 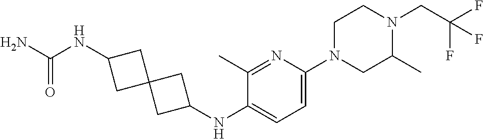 [SMILES: Cc1nc(N2CCN(CC(F)(F)F)C(C)C2)ccc1NC1CC2(CC(NC(N)=O)C2)C1] | 603 |
| 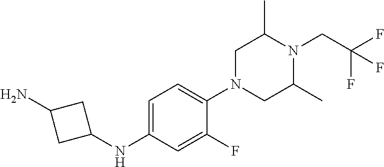 [SMILES: CC1CN(c2ccc(NC3CC(N)C3)cc2F)CC(C)N1CC(F)(F)F] | 604 |
| 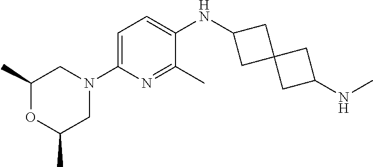 | 605 |
| 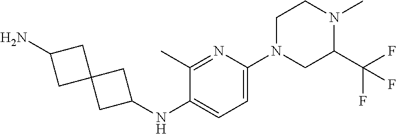 [SMILES: Cc1nc(N2CCN(C)C(C(F)(F)F)C2)ccc1NC1CC2(CC(N)C2)C1] | 606 |
| 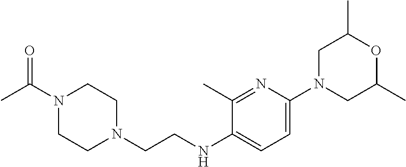 [SMILES: CC(=O)N1CCN(CCNc2ccc(N3CC(C)OC(C)C3)nc2C)CC1] | 607 |
| 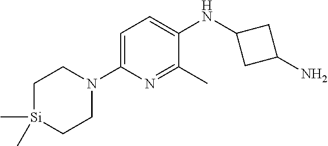 [SMILES: Cc1nc(N2CC[Si](C)(C)CC2)ccc1NC1CC(N)C1] | 608 |
| 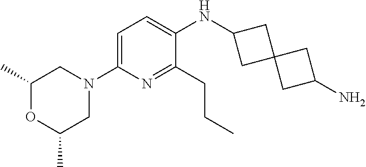 [SMILES: CCCc1nc(N2C[C@@H](C)O[C@@H](C)C2)ccc1NC1CC2(CC(N)C2)C1] | 609 |
| 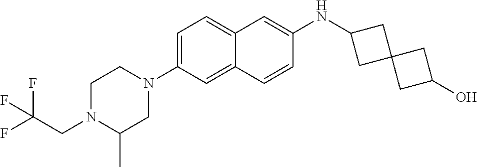 [SMILES: CC1CN(c2ccc3cc(NC4CC5(CC(O)C5)C4)ccc3c2)CCN1CC(F)(F)F] | 610 |
| 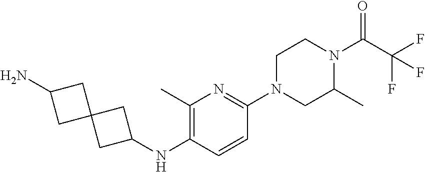 [SMILES: Cc1nc(N2CCN(C(=O)C(F)(F)F)C(C)C2)ccc1NC1CC2(CC(N)C2)C1] | 611 |
| 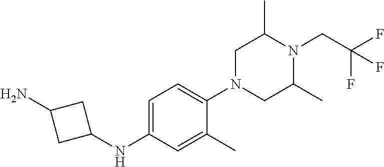 [SMILES: Cc1cc(NC2CC(N)C2)ccc1N1CC(C)N(CC(F)(F)F)C(C)C1] | 612 |
| 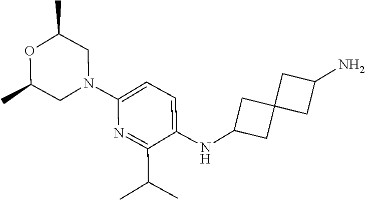 [SMILES: CC(C)c1nc(N2C[C@@H](C)O[C@@H](C)C2)ccc1NC1CC2(CC(N)C2)C1] | 613 |
| 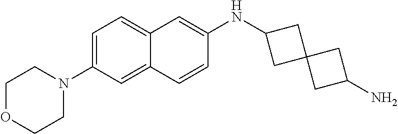 [SMILES: NC1CC2(C1)CC(Nc1ccc3cc(N4CCOCC4)ccc3c1)C2] | 614 |
| 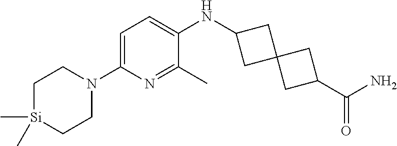 [SMILES: Cc1nc(N2CC[Si](C)(C)CC2)ccc1NC1CC2(C1)CC(C(N)=O)C2] | 615 |
| 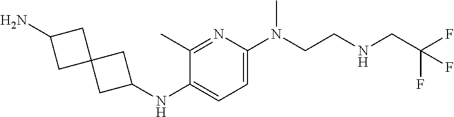 [SMILES: Cc1nc(N(C)CCNCC(F)(F)F)ccc1NC1CC2(CC(N)C2)C1] | 616 |
| 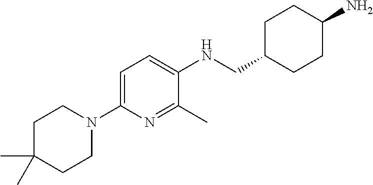 [SMILES: Cc1nc(N2CCC(C)(C)CC2)ccc1NC[C@H]1CC[C@H](N)CC1] | 617 |
| 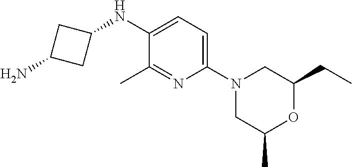 [SMILES: CC[C@@H]1CN(c2ccc(N[C@H]3C[C@@H](N)C3)c(C)n2)C[C@H](C)O1] | 618 |
| 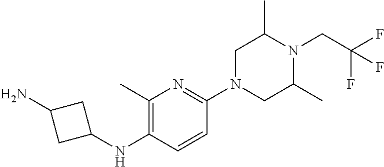 [SMILES: Cc1nc(N2CC(C)N(CC(F)(F)F)C(C)C2)ccc1NC1CC(N)C1] | 619 |
| 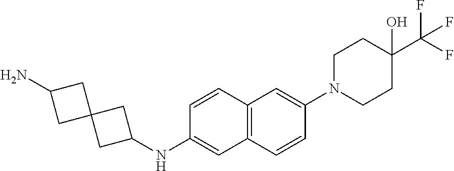 [SMILES: NC1CC2(C1)CC(Nc1ccc3cc(N4CCC(O)(C(F)(F)F)CC4)ccc3c1)C2] | 620 |
| 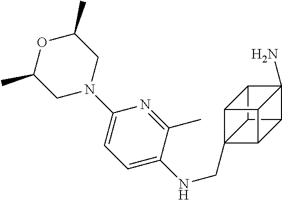 [SMILES: Cc1nc(N2C[C@@H](C)O[C@@H](C)C2)ccc1NCC12C3C4C1C1C2C3C41N] | 621 |
| 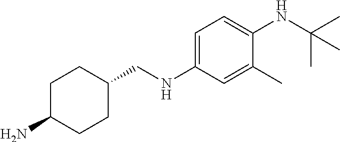 [SMILES: Cc1cc(NC[C@H]2CC[C@H](N)CC2)ccc1NC(C)(C)C] | 622 |
| 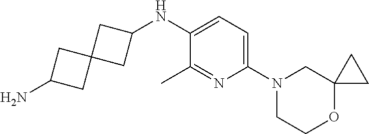 [SMILES: Cc1nc(N2CCOC3(CC3)C2)ccc1NC1CC2(CC(N)C2)C1] | 623 |
| 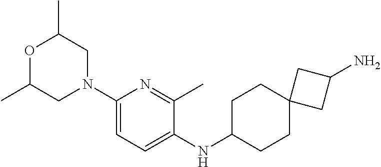 [SMILES: Cc1nc(N2CC(C)OC(C)C2)ccc1NC1CCC2(CC1)CC(N)C2] | 624 |
| 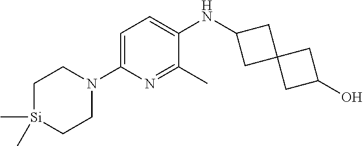 [SMILES: Cc1nc(N2CC[Si](C)(C)CC2)ccc1NC1CC2(CC(O)C2)C1] | 625 |
| 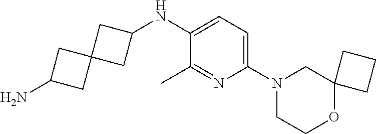 [SMILES: Cc1nc(N2CCOC3(CCC3)C2)ccc1NC1CC2(CC(N)C2)C1] | 626 |
| 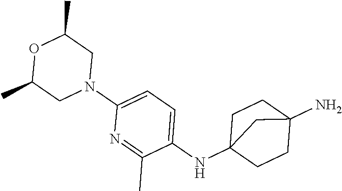 [SMILES: Cc1nc(N2C[C@@H](C)O[C@@H](C)C2)ccc1NC12CCC(N)(CC1)C2] | 627 |
| 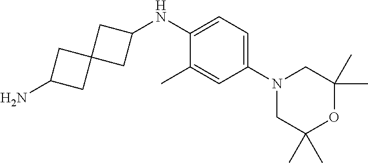 [SMILES: Cc1cc(N2CC(C)(C)OC(C)(C)C2)ccc1NC1CC2(CC(N)C2)C1] | 628 |
| 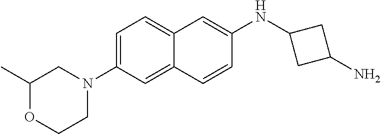 [SMILES: CC1CN(c2ccc3cc(NC4CC(N)C4)ccc3c2)CCO1] | 629 |
| 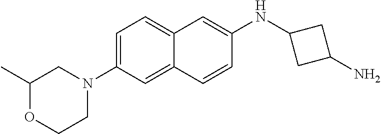 [SMILES: CC1CN(c2ccc3cc(NC4CC(N)C4)ccc3c2)CCO1] | 630 |
| 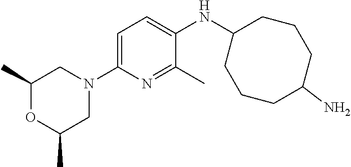 [SMILES: Cc1nc(N2C[C@@H](C)O[C@@H](C)C2)ccc1NC1CCCC(N)CCC1] | 631 |
| 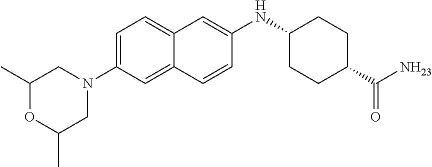 | 632 |
| 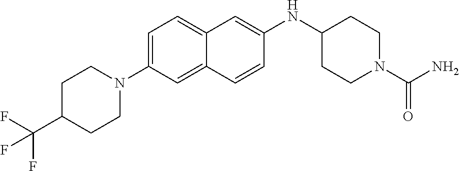 [SMILES: NC(=O)N1CCC(Nc2ccc3cc(N4CCC(C(F)(F)F)CC4)ccc3c2)CC1] | 633 |
| 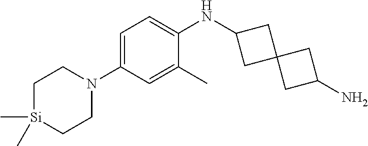 [SMILES: Cc1cc(N2CC[Si](C)(C)CC2)ccc1NC1CC2(CC(N)C2)C1] | 634 |
| 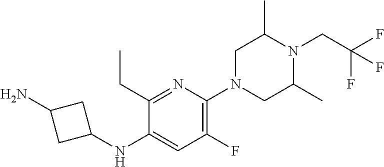 [SMILES: CCc1nc(N2CC(C)N(CC(F)(F)F)C(C)C2)c(F)cc1NC1CC(N)C1] | 635 |
| 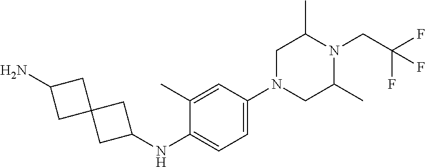 [SMILES: Cc1cc(N2CC(C)N(CC(F)(F)F)C(C)C2)ccc1NC1CC2(CC(N)C2)C1] | 636 |
| 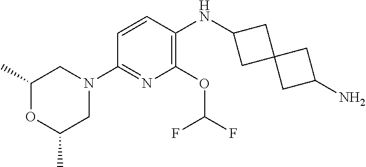 [SMILES: C[C@@H]1CN(c2ccc(NC3CC4(CC(N)C4)C3)c(OC(F)F)n2)C[C@H](C)O1] | 637 |
| 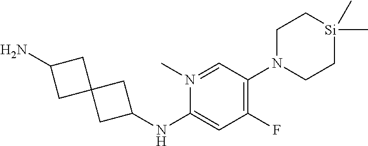 | 638 |
| 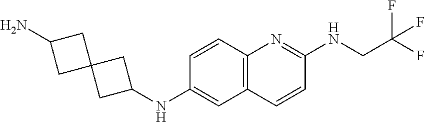 [SMILES: NC1CC2(C1)CC(Nc1ccc3nc(NCC(F)(F)F)ccc3c1)C2] | 639 |
| 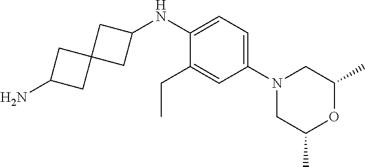 [SMILES: CCc1cc(N2C[C@@H](C)O[C@@H](C)C2)ccc1NC1CC2(CC(N)C2)C1] | 640 |
| 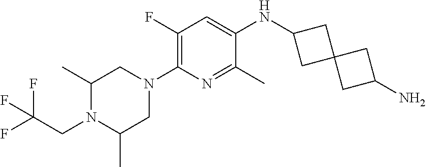 [SMILES: Cc1nc(N2CC(C)N(CC(F)(F)F)C(C)C2)c(F)cc1NC1CC2(CC(N)C2)C1] | 641 |
| 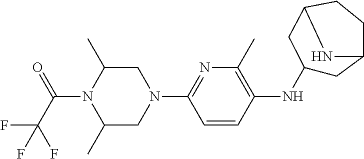 [SMILES: Cc1nc(N2CC(C)N(C(=O)C(F)(F)F)C(C)C2)ccc1NC1CC2CCC(C1)N2] | 642 |
| 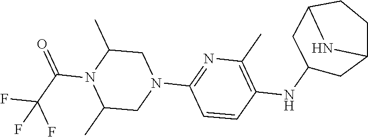 [SMILES: Cc1nc(N2CC(C)N(C(=O)C(F)(F)F)C(C)C2)ccc1NC1CC2CCC(C1)N2] | 643 |
| 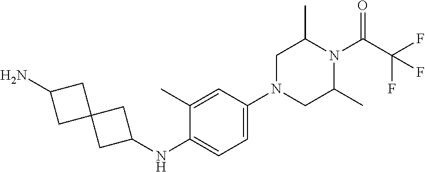 [SMILES: Cc1cc(N2CC(C)N(C(=O)C(F)(F)F)C(C)C2)ccc1NC1CC2(CC(N)C2)C1] | 644 |
| 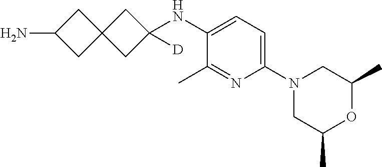 [SMILES: [2H]C1(Nc2ccc(N3C[C@@H](C)O[C@@H](C)C3)nc2C)CC2(CC(N)C2)C1] | 645 |
| 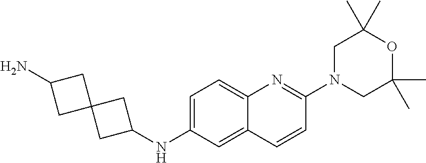 [SMILES: CC1(C)CN(c2ccc3cc(NC4CC5(CC(N)C5)C4)ccc3n2)CC(C)(C)O1] | 646 |
| 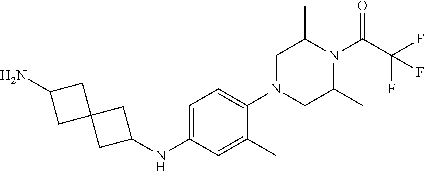 [SMILES: Cc1cc(NC2CC3(CC(N)C3)C2)ccc1N1CC(C)N(C(=O)C(F)(F)F)C(C)C1] | 647 |
| 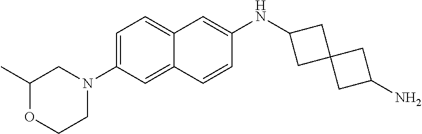 [SMILES: CC1CN(c2ccc3cc(NC4CC5(CC(N)C5)C4)ccc3c2)CCO1] | 648 |
| 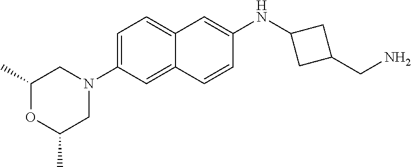 [SMILES: C[C@@H]1CN(c2ccc3cc(NC4CC(CN)C4)ccc3c2)C[C@H](C)O1] | 649 |
| 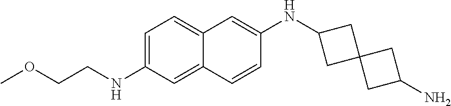 [SMILES: COCCNc1ccc2cc(NC3CC4(CC(N)C4)C3)ccc2c1] | 650 |
| 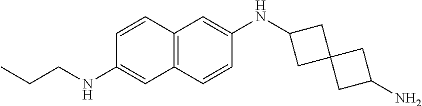 [SMILES: CCCNc1ccc2cc(NC3CC4(CC(N)C4)C3)ccc2c1] | 651 |
| 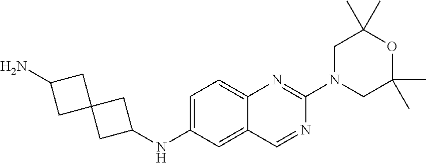 [SMILES: CC1(C)CN(c2ncc3cc(NC4CC5(CC(N)C5)C4)ccc3n2)CC(C)(C)O1] | 652 |
| 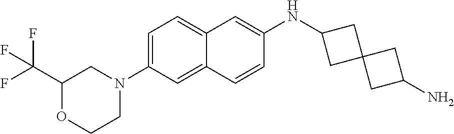 [SMILES: NC1CC2(C1)CC(Nc1ccc3cc(N4CCOC(C(F)(F)F)C4)ccc3c1)C2] | 653 |
| 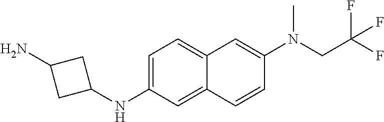 [SMILES: CN(CC(F)(F)F)c1ccc2cc(NC3CC(N)C3)ccc2c1] | 654 |
| 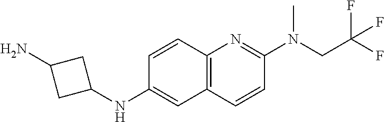 [SMILES: CN(CC(F)(F)F)c1ccc2cc(NC3CC(N)C3)ccc2n1] | 655 |
| 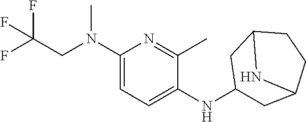 [SMILES: Cc1nc(N(C)CC(F)(F)F)ccc1NC1CC2CCC(C1)N2] | 656 |
| 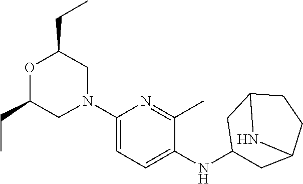 [SMILES: CC[C@@H]1CN(c2ccc(NC3CC4CCC(C3)N4)c(C)n2)C[C@H](CC)O1] | 657 |
| 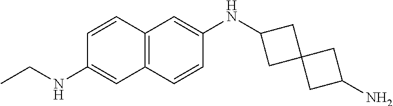 [SMILES: CCNc1ccc2cc(NC3CC4(CC(N)C4)C3)ccc2c1] | 658 |
| 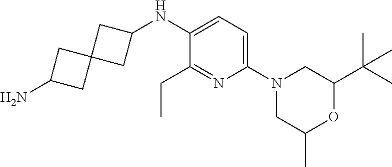 [SMILES: CCc1nc(N2CC(C)OC(C(C)(C)C)C2)ccc1NC1CC2(CC(N)C2)C1] | 659 |
| 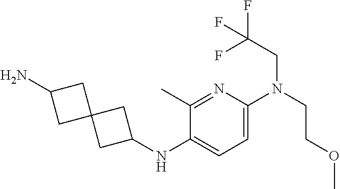 [SMILES: COCCN(CC(F)(F)F)c1ccc(NC2CC3(CC(N)C3)C2)c(C)n1] | 660 |
| 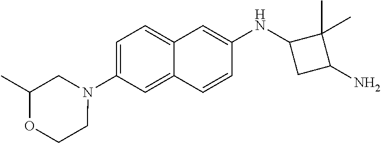 [SMILES: CC1CN(c2ccc3cc(NC4CC(N)C4(C)C)ccc3c2)CCO1] | 661 |

Another aspect of the disclosure provides a pharmaceutical composition comprising at least one compound selected from a compound of the Formulae disclosed herein, Compounds 1 to 573, a tautomer thereof, a solvate or stereoisomer of the compound or the tautomer, or a pharmaceutically acceptable salt of the foregoing, and at least one pharmaceutically acceptable carrier.

Another aspect of the disclosure provides a pharmaceutical composition comprising at least one compound selected from a compound of the Formulae disclosed herein, Compounds 574 to 661, a tautomer thereof, a solvate or stereoisomer of the compound or the tautomer, or a pharmaceutically acceptable salt of the foregoing, and at least one pharmaceutically acceptable carrier.

In some embodiments, the pharmaceutically acceptable carrier is selected from pharmaceutically acceptable vehicles and pharmaceutically acceptable adjuvants. In some embodiments, the pharmaceutically acceptable carrier is chosen from pharmaceutically acceptable fillers, disintegrants, surfactants, binders, and lubricants.

It will also be appreciated that a pharmaceutical composition of this disclosure can be employed in combination therapies; that is, the pharmaceutical compositions described herein can further include an additional active pharmaceutical agent. Alternatively, a pharmaceutical composition comprising a compound selected from a compound of the Formulae disclosed herein, Compounds 1 to 573, a tautomer thereof, a solvate or stereoisomer of the compound or the tautomer, or a pharmaceutically acceptable salt of the foregoing can be administered as a separate composition concurrently with, prior to, or subsequent to, a composition comprising an additional active pharmaceutical agent. It will also be appreciated that a pharmaceutical composition of this disclosure can be employed in combination therapies; that is, the pharmaceutical compositions described herein can further include an additional active pharmaceutical agent. Alternatively, a pharmaceutical composition comprising a compound selected from a compound of the Formulae disclosed herein, Compounds 574 to 661, a tautomer thereof, a solvate or stereoisomer of the compound or the tautomer, or a pharmaceutically acceptable salt of the foregoing can be administered as a separate composition concurrently with, prior to, or subsequent to, a composition comprising an additional active pharmaceutical agent.

In some embodiments, the pharmaceutically acceptable carrier may be chosen from adjuvants and vehicles. The pharmaceutically acceptable carrier, as used herein, can be chosen, for example, from any and all solvents, diluents, other liquid vehicles, dispersion aids, suspension aids, surface active agents, isotonic agents, thickening agents, emulsifying agents, preservatives, solid binders, and lubricants, which are suited to the particular dosage form desired. Remington: The Science and Practice of Pharmacy, 21st edition, 2005, ed. D. B. Troy, Lippincott Williams & Wilkins, Philadelphia, and Encyclopedia of Pharmaceutical Technology, eds. J. Swarbrick and J. C. Boylan, 1988 to 1999, Marcel Dekker, New York discloses various carriers used in formulating pharmaceutical compositions and known techniques for the preparation thereof. Except insofar as any conventional carrier is incompatible with the compounds of this disclosure, such as by producing any undesirable biological effect or otherwise interacting in a deleterious manner with any other component(s) of the pharmaceutical composition, its use is contemplated to be within the scope of this disclosure. Non-limiting examples of suitable pharmaceutically acceptable carriers include ion exchangers, alumina, aluminum stearate, lecithin, serum proteins (such as human serum albumin), buffer substances (such as phosphates, glycine, sorbic acid, and potassium sorbate), partial glyceride mixtures of saturated vegetable fatty acids, water, salts, and electrolytes (such as protamine sulfate, disodium hydrogen phosphate, potassium hydrogen phosphate, sodium chloride, and zinc salts), colloidal silica, magnesium trisilicate, polyvinyl pyrrolidone, polyacrylates, waxes, polyethylene-polyoxypropylene-block polymers, wool fat, sugars (such as lactose, glucose and sucrose), starches (such as corn starch and potato starch), cellulose and its derivatives (such as sodium carboxymethyl cellulose, ethyl cellulose and cellulose acetate), powdered tragacanth, malt, gelatin, talc, excipients (such as cocoa butter and suppository waxes), oils (such as peanut oil, cottonseed oil, safflower oil, sesame oil, olive oil, corn oil and soybean oil), glycols (such as propylene glycol and polyethylene glycol), esters (such as ethyl oleate and ethyl laurate), agar, buffering agents (such as magnesium hydroxide and aluminum hydroxide), alginic acid, pyrogen-free water, isotonic saline, Ringer's solution, ethyl alcohol, phosphate buffer solutions, non-toxic compatible lubricants (such as sodium lauryl sulfate and magnesium stearate), coloring agents, releasing agents, coating agents, sweetening agents, flavoring agents, perfuming agents, preservatives, and antioxidants.

A compound selected from a compound of the Formulae disclosed herein, Compounds 1 to 573, a tautomer thereof, a solvate or stereoisomer of the compound or the tautomer, or a pharmaceutically acceptable salt of the foregoing, or a pharmaceutical composition disclosed herein can be administered orally in solid dosage forms, such as capsules, tablets, troches, dragées, granules and powders, or in liquid dosage forms, such as elixirs, syrups, emulsions, dispersions, and suspensions. The compound, tautomer, solvate, stereoisomer, or pharmaceutically acceptable salt described herein can also be administered parenterally, in sterile liquid dosage forms, such as dispersions, suspensions or solutions. Other dosages forms that can also be used to administer the compound, tautomer, solvate, stereoisomer, or pharmaceutically acceptable salt described herein as an ointment, cream, drops, transdermal patch or powder for topical administration, as an ophthalmic solution or suspension formation, e.g., eye drops, for ocular administration, as an aerosol spray or powder composition for inhalation or intranasal administration, or as a cream, ointment, spray or suppository for rectal or vaginal administration.

A compound selected from a compound of the Formulae disclosed herein, Compounds 574 to 661, a tautomer thereof, a solvate or stereoisomer of the compound or the tautomer, or a pharmaceutically acceptable salt of the foregoing, or a pharmaceutical composition disclosed herein can be administered orally in solid dosage forms, such as capsules, tablets, troches, dragées, granules and powders, or in liquid dosage forms, such as elixirs, syrups, emulsions, dispersions, and suspensions. The compound, tautomer, solvate, stereoisomer, or pharmaceutically acceptable salt described herein can also be administered parenterally, in sterile liquid dosage forms, such as dispersions, suspensions or solutions. Other dosages forms that can also be used to administer the compound, tautomer, solvate, stereoisomer, or pharmaceutically acceptable salt described herein as an ointment, cream, drops, transdermal patch or powder for topical administration, as an ophthalmic solution or suspension formation, e.g., eye drops, for ocular administration, as an aerosol spray or powder composition for inhalation or intranasal administration, or as a cream, ointment, spray or suppository for rectal or vaginal administration.

Gelatin capsules containing a compound, a tautomer thereof, a solvate or stereoisomer of the compound or the tautomer, and/or a pharmaceutically acceptable salt of the foregoing disclosed herein and powdered carriers, such as lactose, starch, cellulose derivatives, magnesium stearate, stearic acid, and the like, can also be used. Similar diluents can be used to make compressed tablets. Both tablets and capsules can be manufactured as sustained release products to provide for continuous release of medication over a period of time. Compressed tablets can be sugar coated or film coated to mask any unpleasant taste and protect the tablet from the atmosphere, or enteric coated for selective disintegration in the gastrointestinal tract. Liquid dosage forms for oral administration can further comprise at least one agent selected from coloring and flavoring agents to increase patient acceptance.

In general, water, a suitable oil, saline, aqueous dextrose (glucose), and related sugar solutions and glycols such as propylene glycol or polyethylene glycols can be examples of suitable carriers for parenteral solutions. Solutions for parenteral administration may comprise a water soluble salt of the at least one compound describe herein, at least one suitable stabilizing agent, and if necessary, at least one buffer substance. Antioxidizing agents such as sodium bisulfite, sodium sulfite, or ascorbic acid, either alone or combined, can be examples of suitable stabilizing agents. Citric acid and its salts and sodium EDTA can also be used as examples of suitable stabilizing agents. In addition, parenteral solutions can further comprise at least one preservative, selected, for example, from benzalkonium chloride, methyl- and propylparaben, and chlorobutanol

A pharmaceutically acceptable carrier is, for example, selected from carriers that are compatible with active ingredients of the composition (and in some embodiments, capable of stabilizing the active ingredients) and not deleterious to the subject to be treated. For example, solubilizing agents, such as cyclodextrins (which can form specific, more soluble complexes with the at least one compound and/or at least one pharmaceutically acceptable salt disclosed herein), can be utilized as pharmaceutical excipients for delivery of the active ingredients. Examples of other carriers include colloidal silicon dioxide, magnesium stearate, cellulose, sodium lauryl sulfate, and pigments such as D&C Yellow #10. Suitable pharmaceutically acceptable carriers are described in Remington's Pharmaceutical Sciences, A. Osol.

For administration by inhalation, the compound, tautomer, solvate, stereoisomer, or pharmaceutically acceptable salt described herein may be conveniently delivered in the form of an aerosol spray presentation from pressurized packs or nebulisers. The compound, tautomer, solvate, stereoisomer, or pharmaceutically acceptable salt described herein may also be delivered as powders, which may be formulated, and the powder composition may be inhaled with the aid of an insufflation powder inhaler device. One exemplary delivery system for inhalation can be metered dose inhalation (MDI) aerosol, which may be formulated as a suspension or solution of a compound, tautomer, solvate, stereoisomer, or pharmaceutically acceptable salt described herein in at least one suitable propellant, selected, for example, from fluorocarbons and hydrocarbons.

For ocular administration, an ophthalmic preparation may be formulated with an appropriate weight percentage of a solution or suspension of the compound, tautomer, solvate, stereoisomer, or pharmaceutically acceptable salt described herein in an appropriate ophthalmic vehicle, such that the compound, tautomer, solvate, stereoisomer, or pharmaceutically acceptable salt described herein is maintained in contact with the ocular surface for a sufficient time period to allow the compound to penetrate the corneal and internal regions of the eye.

Useful pharmaceutical dosage-forms for administration of the compound, tautomer, solvate, stereoisomer, or pharmaceutically acceptable salt described herein include, but are not limited to, hard and soft gelatin capsules, tablets, parenteral injectables, and oral suspensions. In some embodiments, the pharmaceutical compositions disclosed herein may be in the form of controlled release or sustained release compositions as known in the art.

The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient. Typical unit dosage forms include prefilled, premeasured ampules or syringes of the liquid compositions or pills, tablets, capsules, lozenges or the like in the case of solid compositions. In such compositions, the active material is usually a component ranging from about 0.1 to about 50% by weight or preferably from about 1 to about 40% by weight with the remainder being various vehicles or carriers and processing aids helpful for forming the desired dosing form. Unit dosage formulations are preferably about of 5, 10, 25, 50, 100, 250, 500, or 1,000 mg per unit. In a particular embodiment, unit dosage forms are packaged in a multipack adapted for sequential use, such as blisterpack comprising sheets of at least 6, 9 or 12 unit dosage forms.

In some embodiments, unit capsules can be prepared by filling standard two-piece hard gelatin capsules each with, for example, 100 milligrams of the compound, tautomer, solvate, stereoisomer, or pharmaceutically acceptable salt described herein in powder, 150 milligrams of lactose, 50 milligrams of cellulose, and 6 milligrams magnesium stearate.

In some embodiments, a mixture of the compound, tautomer, solvate, stereoisomer, or pharmaceutically acceptable salt described herein and a digestible oil such as soybean oil, cottonseed oil or olive oil can be prepared and injected by means of a positive displacement pump into gelatin to form soft gelatin capsules containing 100 milligrams of the active ingredient. The capsules are washed and dried.

In some embodiments, tablets can be prepared by conventional procedures so that the dosage unit comprises, for example, 100 milligrams of the compound, stereoisomers thereof, or pharmaceutically acceptable salts thereof, 0.2 milligrams of colloidal silicon dioxide, 5 milligrams of magnesium stearate, 275 milligrams of microcrystalline cellulose, 11 milligrams of starch and 98.8 milligrams of lactose. Appropriate coatings may be applied to increase palatability or delay absorption.

In some embodiments, a parenteral composition suitable for administration by injection can be prepared by stirring 1.5% by weight of the compound and/or at least an enantiomer, a diastereoisomer, or pharmaceutically acceptable salt thereof disclosed herein in 10% by volume propylene glycol. The solution is made to the expected volume with water for injection and sterilized.

In some embodiment, an aqueous suspension can be prepared for oral administration. For example, each 5 milliliters of an aqueous suspension comprising 100 milligrams of finely divided compound, stereoisomers thereof, or pharmaceutically acceptable salts thereof, 100 milligrams of sodium carboxymethyl cellulose, 5 milligrams of sodium benzoate, 1.0 grams of sorbitol solution, U.S.P., and 0.025 milliliters of vanillin can be used.

The same dosage forms can generally be used when the compound, tautomer, solvate, stereoisomer, or pharmaceutically acceptable salt described herein is administered stepwise or in conjunction with at least one other therapeutic agent. When drugs are administered in physical combination, the dosage form and administration route should be selected depending on the compatibility of the combined drugs. Thus, the term coadministration is understood to include the administration of at least two agents concomitantly or sequentially, or alternatively as a fixed dose combination of the at least two active components.

The compound, tautomer, solvate, stereoisomer, or pharmaceutically acceptable salt disclosed herein can be administered as the sole active ingredient or in combination with at least one second active ingredient.

The compound, tautomer, solvate, or stereoisomer described herein may be used per se, or in the form of their pharmaceutically acceptable salts, such as hydrochlorides, hydrobromides, acetates, sulfates, citrates, carbonates, trifluoroacetates and the like. When the compound, tautomer, solvate, stereoisomer, or pharmaceutically acceptable salt described herein contain relatively acidic functionalities, salts can be obtained by addition of the desired base, either neat or in a suitable inert solvent. Examples of pharmaceutically acceptable base addition salts include sodium, potassium, calcium, ammonium, organic amino, or magnesium salts, or the like. When the compound, tautomer, solvate, or stereoisomer described herein contain relatively basic functionalities, salts can be obtained by addition of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galacturonic acids and the like (see, for example, Berge et al., “Pharmaceutical Salts,” Journal of Pharmaceutical Science, 1977, 66, 1-19).

Neutral forms of the pharmaceutically acceptable salt described herein may be regenerated by contacting the salt with a base or acid, and isolating the parent compound in the conventional manner.

This disclosure provides prodrugs. Prodrugs of the compound, tautomer, solvate, stereoisomer, or pharmaceutically acceptable salt described herein that readily undergo chemical changes under physiological conditions to provide the compound, tautomer, solvate, stereoisomer, or pharmaceutically acceptable salt of the present disclosure. Additionally, prodrugs can be converted to the compound, tautomer, solvate, stereoisomer, or a pharmaceutically acceptable salt of the present disclosure by chemical or biochemical methods in an ex vivo environment. For example, prodrugs can be slowly converted to the compound, tautomer, solvate, stereoisomer, or pharmaceutically acceptable salt of the present disclosure when placed in a transdermal patch reservoir with a suitable enzyme or chemical reagent. Prodrugs are often useful because, in some situations, they may be easier to administer than the parent drug. They may, for instance, be more bioavailable by oral administration than the parent drug. The prodrug may also have improved solubility in pharmacological compositions over the parent drug. A wide variety of prodrug derivatives are known in the art, such as those that rely on hydrolytic cleavage or oxidative activation of the prodrug. An example, without limitation, of a prodrug would be a compound of the present disclosure which is administered as an ester (the “prodrug”), but then is metabolically hydrolyzed to the carboxylic acid, i.e., the active entity.

Certain compound, tautomer, stereoisomer, or pharmaceutically acceptable salt of the disclosure can exist in unsolvated forms as well as solvated forms, including hydrate forms. Certain compound, tautomer, solvate, stereoisomer, or pharmaceutically acceptable salt of the disclosure may exist in multiple crystalline or amorphous forms.

Certain compound, tautomer, solvate, or pharmaceutically acceptable salt in this disclosure possesses asymmetric carbon atoms (optical centers) or double bonds; the racemates, enantiomers, diastereoisomers, geometric isomers and individual isomers are all intended to be encompassed within the scope of the present disclosure.

### III. Methods of Treatment and Uses

Another aspect of the disclosure provides a method of treating a disease or condition, comprising administering to a subject in need thereof, a therapeutically effective amount of a compound of Formulae disclosed herein, a tautomer thereof, a solvate or stereoisomer of the compound or the tautomer, or a pharmaceutically acceptable salt of the foregoing, or a pharmaceutical composition comprising any of the foregoing, wherein the disease or condition is selected from neuropathy, stroke, neurodegenerative disease (e.g., Alzheimer's disease), Parkinson's Disease, amyotrophic lateral sclerosis (AML), multiple sclerosis, Huntington's Disease, dementia with Lewy bodies, Friedreich's ataxia, hair follicle morphogenesis, diabetes, sepsis, transplant rejection, periventricular leukomalacia, ischemia reperfusion injury, blood coagulation, myocardial infarction, and kidney dysfunction such as acute kidney failure. In some embodiments, the disease or condition is involved with dysregulated ferroptosis, e.g., abnormal ferroptosis

In another aspect, disclosed herein is a compound, tautomer, solvate, stereoisomer, or pharmaceutically acceptable salt as described herein, including a compound of the Formulae disclosed herein, Compounds 1 to 573, a tautomer thereof, a solvate or stereoisomer of the compound or the tautomer, or a pharmaceutically acceptable salt of the foregoing, or a pharmaceutical composition thereof, for use as a medicament.

In another aspect, disclosed herein is a compound, tautomer, solvate, stereoisomer, or pharmaceutically acceptable salt as described herein, including a compound of the Formulae disclosed herein, Compounds 574 to 661, a tautomer thereof, a solvate or stereoisomer of the compound or the tautomer, or a pharmaceutically acceptable salt of the foregoing, or a pharmaceutical composition thereof, for use as a medicament.

In another aspect, disclosed herein is use of a compound, tautomer, solvate, stereoisomer, or pharmaceutically acceptable salt as described herein, including a compound of the Formulae disclosed herein, Compounds 1 to 573, a tautomer thereof, a solvate or stereoisomer of the compound or the tautomer, or a pharmaceutically acceptable salt of the foregoing, or a pharmaceutical composition thereof, for the manufacture of a medicament for treating a disease or condition selected from neuropathy, stroke, neurodegenerative disease (e.g., Alzheimer's disease), Parkinson's Disease, amyotrophic lateral sclerosis (AML), multiple sclerosis, Huntington's Disease, dementia with Lewy bodies, Friedreich's ataxia, hair follicle morphogenesis, diabetes, sepsis, transplant rejection, periventricular leukomalacia, ischemia reperfusion injury, blood coagulation, myocardial infarction, and kidney dysfunction such as acute kidney failure. In some embodiments, the disease or condition is involved with dysregulated ferroptosis, e.g., abnormal ferroptosis. In another aspect, disclosed herein is use of a compound, tautomer, solvate, stereoisomer, or pharmaceutically acceptable salt as described herein, including a compound of the Formulae disclosed herein, Compounds 574 to 661, a tautomer thereof, a solvate or stereoisomer of the compound or the tautomer, or a pharmaceutically acceptable salt of the foregoing, or a pharmaceutical composition thereof, for the manufacture of a medicament for treating a disease or condition selected from neuropathy, stroke, neurodegenerative disease (e.g., Alzheimer's disease), Parkinson's Disease, amyotrophic lateral sclerosis (AML), multiple sclerosis, Huntington's Disease, dementia with Lewy bodies, Friedreich's ataxia, hair follicle morphogenesis, diabetes, sepsis, transplant rejection, periventricular leukomalacia, ischemia reperfusion injury, blood coagulation, myocardial infarction, and kidney dysfunction such as acute kidney failure. In some embodiments, the disease or condition is involved with dysregulated ferroptosis, e.g., abnormal ferroptosis.

In a further aspect of this disclosure, a compound, tautomer, solvate, stereoisomer, or pharmaceutically acceptable salt as described herein, including a compound of the Formulae disclosed herein, Compounds 1 to 573, a tautomer thereof, a solvate or stereoisomer of the compound or the tautomer, or a pharmaceutically acceptable salt of the foregoing, or a pharmaceutical composition thereof, is for use in treating a disease or condition selected from neuropathy, stroke, neurodegenerative disease (e.g., Alzheimer's disease), Parkinson's Disease, amyotrophic lateral sclerosis (AML), multiple sclerosis, Huntington's Disease, dementia with Lewy bodies, Friedreich's ataxia, hair follicle morphogenesis, diabetes, sepsis, transplant rejection, periventricular leukomalacia, ischemia reperfusion injury, blood coagulation, myocardial infarction, and kidney dysfunction such as acute kidney failure. In some embodiments, the disease or condition is involved with dysregulated ferroptosis, e.g., abnormal ferroptosis

In a further aspect of this disclosure, a compound, tautomer, solvate, stereoisomer, or pharmaceutically acceptable salt as described herein, including a compound of the Formulae disclosed herein, Compounds 574 to 661, a tautomer thereof, a solvate or stereoisomer of the compound or the tautomer, or a pharmaceutically acceptable salt of the foregoing, or a pharmaceutical composition thereof, is for use in treating a disease or condition selected from neuropathy, stroke, neurodegenerative disease (e.g., Alzheimer's disease), Parkinson's Disease, amyotrophic lateral sclerosis (AML), multiple sclerosis, Huntington's Disease, dementia with Lewy bodies, Friedreich's ataxia, hair follicle morphogenesis, diabetes, sepsis, transplant rejection, periventricular leukomalacia, ischemia reperfusion injury, blood coagulation, myocardial infarction, and kidney dysfunction such as acute kidney failure. In some embodiments, the disease or condition is involved with dysregulated ferroptosis, e.g., abnormal ferroptosis

Another aspect of the disclosure provides a method of modulating, e.g., inhibiting, ferroptosis in a subject in need thereof, comprising administering to the subject, a therapeutically effective amount of a compound of Formulae disclosed herein, a tautomer thereof, a solvate or stereoisomer of the compound or the tautomer, or a pharmaceutically acceptable salt of the foregoing, or a pharmaceutical composition comprising any of the foregoing.

In another aspect, disclosed herein is use of a compound, tautomer, solvate, stereoisomer, or pharmaceutically acceptable salt as described herein, including a compound of the Formulae disclosed herein, Compounds 1 to 573, a tautomer thereof, a solvate or stereoisomer of the compound or the tautomer, or a pharmaceutically acceptable salt of the foregoing, or a pharmaceutical composition thereof, for modulating, e.g., inhibiting, ferroptosis in a subject in need thereof.

In another aspect, disclosed herein is use of a compound, tautomer, solvate, stereoisomer, or pharmaceutically acceptable salt as described herein, including a compound of the Formulae disclosed herein, Compounds 574 to 661, a tautomer thereof, a solvate or stereoisomer of the compound or the tautomer, or a pharmaceutically acceptable salt of the foregoing, or a pharmaceutical composition thereof, for modulating, e.g., inhibiting, ferroptosis in a subject in need thereof.

In another aspect of this disclosure, a compound, tautomer, a solvate or stereoisomer of the compound or the tautomer, or pharmaceutically acceptable salt as described herein, including a compound of the Formulae disclosed herein, Compounds 1 to 573, a tautomer thereof, a solvate or stereoisomer of the compound or the tautomer, or a pharmaceutically acceptable salt of the foregoing, or a pharmaceutical composition thereof, is for use in modulating, e.g., inhibiting, ferroptosis in a subject in need thereof by contacting the subject with the compound, tautomer, a solvate or stereoisomer of the compound or the tautomer, pharmaceutically acceptable salt, or pharmaceutical composition.

In another aspect of this disclosure, a compound, tautomer, a solvate or stereoisomer of the compound or the tautomer, or pharmaceutically acceptable salt as described herein, including a compound of the Formulae disclosed herein, Compounds 574 to 661, a tautomer thereof, a solvate or stereoisomer of the compound or the tautomer, or a pharmaceutically acceptable salt of the foregoing, or a pharmaceutical composition thereof, is for use in modulating, e.g., inhibiting, ferroptosis in a subject in need thereof by contacting the subject with the compound, tautomer, a solvate or stereoisomer of the compound or the tautomer, pharmaceutically acceptable salt, or pharmaceutical composition.

A compound of the Formulae disclosed herein, Compounds 1 to 573, a tautomer thereof, a solvate or stereoisomer of the compound or the tautomer, or a pharmaceutically acceptable salt of the foregoing, or a pharmaceutical composition thereof may be administered once daily, twice daily, or three times daily, for example, for the treatment of a disease or condition, selected from neuropathy, stroke, neurodegenerative disease (e.g., Alzheimer's disease), Parkinson's Disease, amyotrophic lateral sclerosis (AML), multiple sclerosis, Huntington's Disease, dementia with Lewy bodies, Friedreich's ataxia, hair follicle morphogenesis, diabetes, sepsis, transplant rejection, periventricular leukomalacia, ischemia reperfusion injury, blood coagulation, myocardial infarction, and kidney dysfunction such as acute kidney failure. In some embodiments, the disease or condition is involved with dysregulated ferroptosis, e.g., abnormal ferroptosis.

A compound of the Formulae disclosed herein, Compounds 574 to 661, a tautomer thereof, a solvate or stereoisomer of the compound or the tautomer, or a pharmaceutically acceptable salt of the foregoing, or a pharmaceutical composition thereof may be administered once daily, twice daily, or three times daily, for example, for the treatment of a disease or condition, selected from neuropathy, stroke, neurodegenerative disease (e.g., Alzheimer's disease), Parkinson's Disease, amyotrophic lateral sclerosis (AML), multiple sclerosis, Huntington's Disease, dementia with Lewy bodies, Friedreich's ataxia, hair follicle morphogenesis, diabetes, sepsis, transplant rejection, periventricular leukomalacia, ischemia reperfusion injury, blood coagulation, myocardial infarction, and kidney dysfunction such as acute kidney failure. In some embodiments, the disease or condition is involved with dysregulated ferroptosis, e.g., abnormal ferroptosis.

A compound of the Formulae disclosed herein, Compounds 1 to 573, a tautomer thereof, a solvate or stereoisomer of the compound or the tautomer, or a pharmaceutically acceptable salt of the foregoing, or a pharmaceutical composition thereof may be administered, for example, various manners, such as orally, topically, rectally, parenterally, by inhalation spray, or via an implanted reservoir, although the most suitable route in any given case will depend on the particular host, and nature and severity of the conditions for which the active ingredient is being administered. The term “parenteral” as used herein includes subcutaneous, intracutaneous, intravenous, intramuscular, intraarticular, intraarterial, intrasynovial, intrasternal, intrathecal, intralesional and intracranial injection or infusion techniques. The compositions disclosed herein may be conveniently presented in unit dosage form and prepared by any of the methods well known in the art. Parenteral administration can be by continuous infusion over a selected period of time. Other forms of administration contemplated in this disclosure are as described in International Patent Application Nos. WO 2013/075083, WO 2013/075084, WO 2013/078320, WO 2013/120104, WO 2014/124418, WO 2014/151142, and WO 2015/023915.

A compound of the Formulae disclosed herein, Compounds 574 to 661, a tautomer thereof, a solvate or stereoisomer of the compound or the tautomer, or a pharmaceutically acceptable salt of the foregoing, or a pharmaceutical composition thereof may be administered, for example, various manners, such as orally, topically, rectally, parenterally, by inhalation spray, or via an implanted reservoir, although the most suitable route in any given case will depend on the particular host, and nature and severity of the conditions for which the active ingredient is being administered. The term “parenteral” as used herein includes subcutaneous, intracutaneous, intravenous, intramuscular, intraarticular, intraarterial, intrasynovial, intrasternal, intrathecal, intralesional and intracranial injection or infusion techniques. The compositions disclosed herein may be conveniently presented in unit dosage form and prepared by any of the methods well known in the art. Parenteral administration can be by continuous infusion over a selected period of time. Other forms of administration contemplated in this disclosure are as described in International Patent Application Nos. WO 2013/075083, WO 2013/075084, WO 2013/078320, WO 2013/120104, WO 2014/124418, WO 2014/151142, and WO 2015/023915.

The contacting is generally effected by administering to the subject an effective amount of one or more compounds, tautomers, solvates, stereoisomers, and pharmaceutically acceptable salt disclosed herein. Generally, administration is adjusted to achieve a therapeutic dosage of about 0.1 to 50, preferably 0.5 to 10, more preferably 1 to 10 mg/kg, though optimal dosages are compound specific, and generally empirically determined for each compound.

The dosage administered will be dependent on factors, such as the age, health and weight of the recipient, the extent of disease, type of concurrent treatment, if any, frequency of treatment, and the nature of the effect desired. In general, a daily dosage of the active ingredient can vary, for example, from 0.1 to 2000 milligrams per day. For example, 10-500 milligrams once or multiple times per day may be effective to obtain the desired results.

In some embodiments, 2 mg to 1500 mg or 5 mg to 1000 mg of a compound of the Formulae disclosed herein, Compounds 1 to 573, a tautomer thereof, a solvate or stereoisomer of the compound or the tautomer, or a pharmaceutically acceptable salt of the foregoing, or a pharmaceutical composition thereof are administered once daily, twice daily, or three times daily. The compound, tautomer, solvate, stereoisomer, or pharmaceutically acceptable salt described herein is administered for morning/daytime dosing, with off period at night.

In some embodiments, 2 mg to 1500 mg or 5 mg to 1000 mg of a compound of the Formulae disclosed herein, Compounds 574 to 661, a tautomer thereof, a solvate or stereoisomer of the compound or the tautomer, or a pharmaceutically acceptable salt of the foregoing, or a pharmaceutical composition thereof are administered once daily, twice daily, or three times daily. The compound, tautomer, solvate, stereoisomer, or pharmaceutically acceptable salt described herein is administered for morning/daytime dosing, with off period at night.

### EXAMPLES

In order that the disclosure described herein may be more fully understood, the following examples are disclosed herein. It should be understood that these examples are for illustrative purposes only and are not to be construed as limiting this disclosure in any way.

### Example 1. Synthesis of Exemplary Compounds

The compounds of the disclosure, selected from a compound of the Formulae depicted herein, a tautomer thereof, a solvate or stereoisomer of the compound or the tautomer, or a pharmaceutically acceptable salt of the foregoing, can be made according to standard chemical practices or as illustrated herein, including the following synthetic schemes for Compounds 1 to 661 as representative examples of Formula I.

### Compound 1

### 1-ethyl-5-oxo-N-((4-((4-(4-(trifluoromethyl)piperidin-1-yl)phenyl)amino)cyclohexyl)methyl) pyr rolidine-3-carboxamide

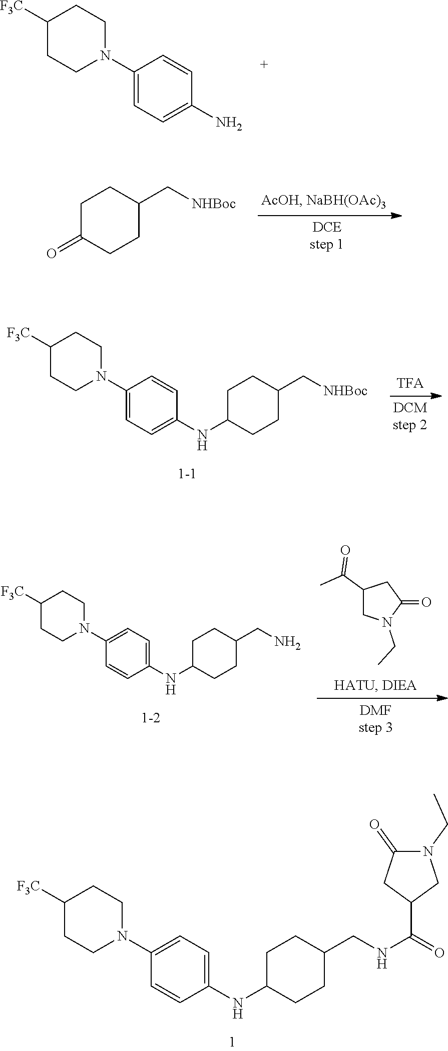 [SMILES: Nc1ccc(N2CCC(C(F)(F)F)CC2)cc1 ; CC(C)(C)OC(=O)NCC1CCC(=O)CC1 ; CC(C)(C)OC(=O)NCC1CCC(Nc2ccc(N3CCC(C(F)(F)F)CC3)cc2)CC1 ; NCC1CCC(Nc2ccc(N3CCC(C(F)(F)F)CC3)cc2)CC1 ; CCN1CC(C(C)=O)CC1=O ; CCN1CC(C(=O)NCC2CCC(Nc3ccc(N4CCC(C(F)(F)F)CC4)cc3)CC2)CC1=O]

Step 1. Preparation of tert-butyl ((4-((4-(4-(trifluoromethyl)piperidin-1-yl)phenyl)amino)cyclohexyl)methyl)carbamate(1-1) A solution of 4-(4-(trifluoromethyl)piperidin-1-yl)aniline (100 mg, 0.41 mmol), tert-butyl ((4-oxocyclohexyl)methyl)carbamate (140 mg, 0.62 mmol) and AcOH (0.1 mL) in DCE (10 mL) was stirred at room temperature for 30 min. NaBH (OAc) 3 (174 mg, 0.82 mmol) was added, and the mixture was stirred at room temperature for 2 h. The mixture was extracted by DCM (25 mL×3). The combined organic layers were washed with brine (15 mL×3), dried over Na₂SO₄ and concentrated to give the crude product, which was purified by TLC (MeOH/DCM=1/20) to give the desired product (130 mg, 69.9%) as a purple solid. Mass (m/z): 456.3 [M+H]⁺.

Step 2. Preparation of N-(4-(aminomethyl)cyclohexyl)-4-(4-(trifluoromethyl)piperidin-1-yl)aniline (1-2) To a solution of 1-1 (130 mg, 0.29 mmol) in DCM (10 mL) was added TFA (1 mL). The solution was stirred at room temperature for 1 h. The mixture was basified by 1M NaOH to pH=9, concentrated, and then extracted by DCM (25 mL×3). The combined organic layers were washed with brine (15 mL×3), dried over Na₂SO₄ and concentrated, which was purified by TLC (MeOH/DCM=1/5) to give the desired product (79.2 mg, 78.2%) as a purple solid. Mass (m/z): 356.3 [M+H]⁺.

Step 3. Preparation of 1-ethyl-5-oxo-N-((4-((4-(4-(trifluoromethyl)piperidin-1-yl)phenyl)amino)cyclohexyl)methyl) p yrrolidine-3-carboxamide (1)

To a solution of 1-2 (50 mg, 0.141 mmol), 1-ethyl-5-oxopyrrolidine-3-carboxylic acid (28 mg, 0.183 mmol), HATU (33 mg, 0.183 mmol) and DIEA (23 mg, 0183 mmol) in DMF (5 mL) was stirred at room temperature for 2 h. The mixture was extracted by EA (25 mL×3). The combined organic layers were washed with brine (15 mL×3), dried over Na₂SO₄ and concentrated to give the crude product, which was purified by TLC (MeOH/DCM=1/10) to give the desired product (23.7 mg, 34.0%) as a purple solid. ¹H NMR (400 MHZ, DMSO-d₆) δ 8.05 (t, J=5.6 Hz, 1H), 6.74 (d, J=8.4 Hz, 2H), 6.47 (d, J=8.4 Hz, 2H), 4.87 (s, 1H), 3.50 (t, J=9.2 Hz, 1H), 3.41 (d, J=14.4 Hz, 2H), 3.33-3.30 (m, 1H), 3.22-3.15 (m, 2H), 3.13-3.06 (m, 1H), 3.04 (d, J=12.0 Hz, 1H), 2.94 (t, J=6.4 Hz, 2H), 2.55 (s, 1H), 2.38 (dd, J=8.8, 3.0 Hz, 3H), 1.96 (d, J=10.4 Hz, 2H), 1.85 (d, J=12.4 Hz, 2H), 1.71 (d, J=10.8 Hz, 2H), 1.57 (tt, J=12.8, 6.4 Hz, 2H), 1.37 (s, 1H), 1.06-0.93 (m, 7H). Mass (m/z): 495.3 [M+H]⁺.

### Compound 2

### 5-oxo-N-((4-((4-(4-(trifluoromethyl)piperidin-1-yl)phenyl)amino)cyclohexyl)methyl)pyrrolidine-3-carboxamide

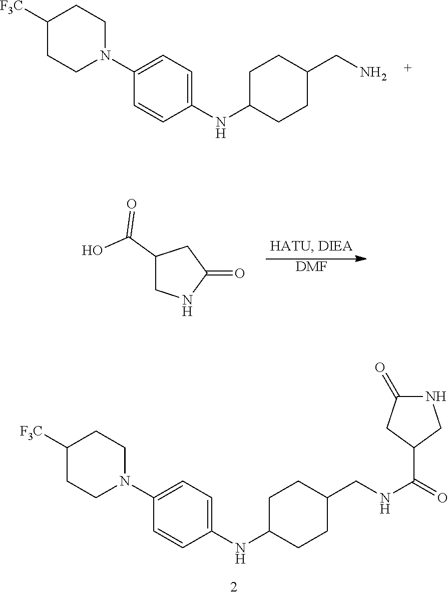

The title compound 2 (23.7 mg) was prepared in a total yield of 34.0% as a purple solid from trifluoromethyl)piperidin-1-yl)aniline (50 mg, 0.141 mmol), 5-oxopyrrolidine-3-carboxylic acid (24 mg, 0.183 mmol), HATU (33 mg, 0.183 mmol) and DIEA (23 mg, 0183 mmol) in DMF (5 mL) according to the procedure for 1. ¹H NMR (400 MHZ, DMSO-d₆) δ 7.95 (s, 1H), 7.50 (s, 1H), 6.86-6.38 (m, 4H), 3.34 (s, 2H), 3.16-2.94 (m, 4H), 2.85 (t, J=6.4 Hz, 2H), 2.33 (s, 1H), 2.20 (dd, J=8.4, 1.6 Hz, 2H), 1.88 (d, J=11.4 Hz, 2H), 1.79 (d, J=12.4 Hz, 2H), 1.64 (d, J=12.0 Hz, 2H), 1.49 (s, 2H), 1.30 (d, J=7.6 Hz, 1H), 1.24-1.13 (m, 2H), 1.02 (s, 1H), 0.91 (t, J=12.0 Hz, 2H). Mass (m/z): 466.55 [M+H]⁺.

### Compound 3

### N-(4-(aminomethyl)cyclohexyl)-4-(4-(trifluoromethyl)piperidin-1-yl)aniline

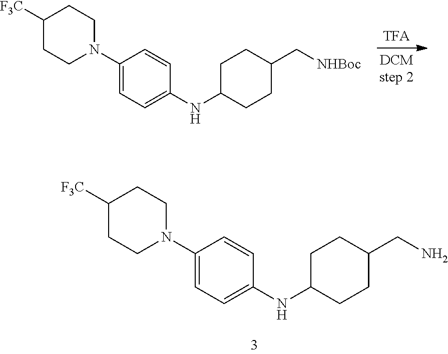 [SMILES: CC(C)(C)OC(=O)NCC1CCC(Nc2ccc(N3CCC(C(F)(F)F)CC3)cc2)CC1 ; NCC1CCC(Nc2ccc(N3CCC(C(F)(F)F)CC3)cc2)CC1]

To a solution of tert-butyl ((4-((4-(4-(trifluoromethyl)piperidin-1-yl)phenyl)amino)cyclohexyl)methyl)carbamate(1-1; 130 mg, 0.29 mmol) in DCM (10 mL) was added TFA (1 mL). The solution was stirred at room temperature for 1 h. The mixture was basified by 1M NaOH to pH=9 and concentrated, then extracted by DCM (25 mL×3). The combined organic layers were washed with brine (15 mL×3), dried over Na₂SO₄ and concentrated, which was purified by TLC (MeOH/DCM=1/5) to give the desired product as a purple solid (79.2 mg, 78.2%) with 1:2 mixture by 1H NMR. ¹H NMR (400 MHZ, DMSO-d₆) δ 7.91 (s, 2H), 6.75 (dd, J=9.2, 2.4 Hz, 2H), 6.57-6.51 (m, 1.4H), 6.50-6.45 (m, 0.7H), 4.95 (d, J=29.2 Hz, 1H), 3.44 (t, J=3.2 Hz, 4H), 2.68 (d, J=6.8 Hz, 1.4H), 2.63 (d, J=6.8 Hz, 0.7H), 2.55 (d, J=2.4 Hz, 2H), 2.42-2.30 (m, 1H), 1.98 (d, J=8.8 Hz, 1H), 1.89-1.81 (m, 2H), 1.71-1.36 (m, 9H). Mass (m/z): 356.3 [M+H]⁺.

### Compound 4

### N-(4-methylcyclohexyl)-4-(4-(trifluoromethyl)piperidin-1-yl)aniline

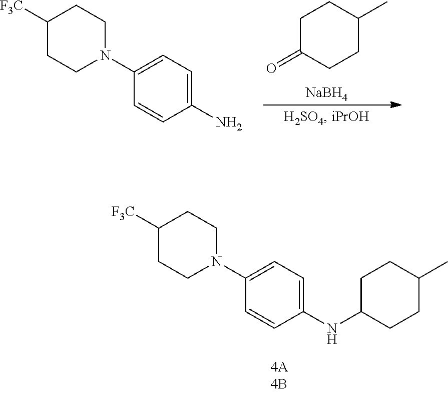

To a solution of 4-(4-(trifluoromethyl)piperidin-1-yl)aniline (100 mg, 0.4 mmol) and 4-methylcyclohexan-1-one (55 mg, 0.49 mmol) in isopropanol (5 mL) was added 3 drops of sulfuric acid dropwise at 0° C. The mixture was stirred for 30 min at room temperature before NaBH₄ (31 mg, 0.8 mmol) was added slowly at 0° C. The resulting mixture was stirred overnight. The reaction was quenched with saturated NaHCO₃ aqueous solution and extracted with dichloromethane (5 mL) 3 times. The organic layers were combined and washed with water, sat. NaHCO₃, and brine respectively, dried over MgSO₄, filtered, and concentrated under reduced pressure. The residue was purified by silica gel column chromatography (petroleum ether/AcOEt, 4/1) and then purified by Prep-HPLC (Column: X Select-CSH-Prep 5 μm OBD, 19*150 mm; ACN/water (0.5% TFA)=5%-5%-95%-95%-5%, 0 min-1 min-10 min-11 min-15.0 min) to give 33.4 mg of 4A (Rt=6.05 min) as a white powder in a yield of 23.9% and 20.4 mg of 4B (Rt=6.16 min) as a white powder in a yield of 14.6%. 4A: ¹H NMR (400 MHz, DMSO-d₆) δ 6.73 (d, J=8.9 Hz, 2H), 6.52 (d, J=8.9 Hz, 2H), 4.94 (s, 1H), 3.41 (d, J=12.0 Hz, 2H), 2.54 (d, J=2.6 Hz, 2H), 2.35 (m, 1H), 1.89-1.79 (m, 2H), 1.54 (m, 7H), 1.42 (m, 2H), 1.38-1.26 (m, 2H), 1.23 (s, 1H), 0.89 (d, J=6.6 Hz, 3H). Mass (m/z): 341.3 [M+H]⁺. 4B: ¹H NMR (400 MHZ, DMSO-d₆) δ 6.73 (d, J=8.4 Hz, 2H), 6.47 (d, J=8.4 Hz, 2H), 4.85 (s, 1H), 3.41 (d, J=11.9 Hz, 2H), 3.00 (s, 1H), 2.43-2.22 (m, 1H), 1.89 (dd, J=31.5, 12.0 Hz, 4H), 1.67 (d, J=12.0 Hz, 2H), 1.56 (qd, J=12.4, 3.9 Hz, 2H), 1.40-1.25 (m, 1H), 1.14-0.93 (m, 4H), 0.87 (d, J=6.5 Hz, 3H). Mass (m/z): 341.3 [M+H]⁺.

### Compound 5

### 1-ethyl-N-(4-(4-(trifluoromethyl)piperidin-1-yl)phenyl)piperidin-4-amine

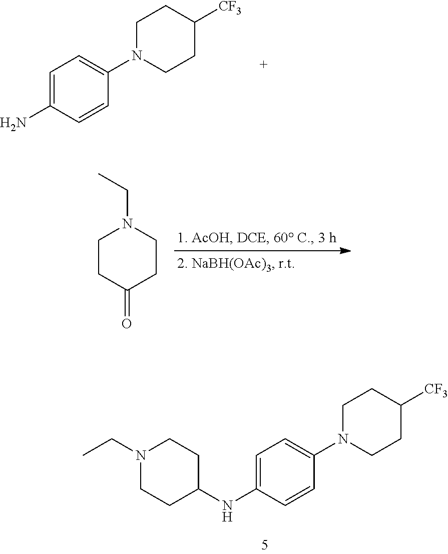

To a solution of 4-(4-(trifluoromethyl)piperidin-1-yl)aniline (150 mg, 0.61 mmol) in DCE (5 ml) was added 1-ethylpiperidin-4-one (94 mg, 0.73 mmol) and acetic acid (3.7 mg, 0.061 mmol). The resulting mixture was stirred at 60° C. for 3 h, and then cooled to room temperature. Sodium triacetoxyborohydride (388 mg, 1.83 mmol) was added and the reaction was stirred for 3 h at R.T. The reaction was quenched with water (10 mL), and extracted by EA (10 mL) for 3 times. The combined organic phase was dried over sodium sulfate, and removed under vacuum.

The residue was purified by prep-HPLC (column-Xbridge-C18 150×21.2 mm, 5 um; Mobile phase: ACN-H₂O (0.1% FA), 40%-60%) to afford the desired product (35 mg, 16.2%) as a white solid. ¹H NMR (300 MHz, CDCl₃) δ 6.84 (d, J=9.0 Hz, 2H), 6.59 (d, J=9.0 Hz, 2H), 3.52 (d, J=12.1 Hz, 2H), 3.35 (s, 2H), 2.93 (d, J=9.0 Hz, 2H), 2.70 (d, J=10.8 Hz, 2H), 2.59 (t, J=11.8 Hz, 2H), 2.26 (s, 2H), 2.06-1.89 (m, 5H), 1.86-1.68 (m, 3H), 1.40 (t, J=9.0 Hz, 3H). Mass (m/z): 356.2 [M+H]⁺.

### Compound 6

### methyl 4-((4-(4-(trifluoromethyl)piperidin-1-yl)phenyl)amino)cyclohexane-1-carboxylate

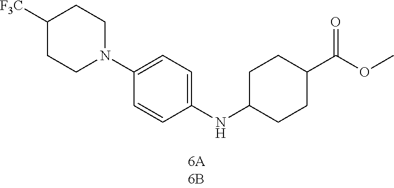 [SMILES: COC(=O)C1CCC(Nc2ccc(N3CCC(C(F)(F)F)CC3)cc2)CC1]

The title compound 6A (Rt=5.37 min; 89.3 mg, 28.4%) as a rosy brown solid and 6B (Rt=5.31 min; 93.6 mg, 29.7%) as a rosy brown solid were prepared from 4-(4-(trifluoromethyl)piperidin-1-yl)aniline (200 mg, 0.82 mmol), methyl 4-oxocyclohexane-1-carboxylate (192 mg, 1.23 mmol) and NaBH (OAc) 3 (347 mg, 1.64 mmol) according to the procedure for 4, which were purified by Prep-HPLC (Column: X Select-CSH-Prep 5 μm OBD, 19*150 mm; ACN/water (0.5% TFA)=5%-5%-95%-95%-5%, 0 min-1 min-10 min-11 min-15.0 min). 6A: ¹H NMR (400 MHZ, DMSO-d₆) δ 6.74 (d, J=8.9 Hz, 2H), 6.51 (d, J=8.9 Hz, 2H), 5.01 (d, J=7.8 Hz, 1H), 3.60 (s, 3H), 3.41 (d, J=12.1 Hz, 2H), 3.29 (d, J=8.1 Hz, 1H), 2.56-2.51 (m, 2H), 2.42-2.28 (m, 1H), 1.93-1.81 (m, 4H), 1.53-1.72 (m, 6H), 1.52-1.39 (m, 3H). Mass (m/z): 385.4 [M+H]⁺. 6B: ¹H NMR (400 MHZ, DMSO-d₆) δ 6.74 (d, J=8.5 Hz, 2H), 6.49 (d, J=8.8 Hz, 2H), 4.90 (s, 1H), 3.59 (s, 3H), 3.42 (dd, J=14.7, 5.8 Hz, 2H), 3.07 (s, 1H), 2.55 (s, 2H), 2.46-2.35 (m, 3H), 2.31-2.19 (m, 4H), 2.19-2.06 (m, 3H), 1.56 (qd, J=12.3, 3.9 Hz, 2H), 1.45 (qd, J=13.1, 3.1 Hz, 2H). Mass (m/z): 385.4 [M+H]⁺.

### Compound 7

### N-isopropyl-4-(4-(trifluoromethyl)piperidin-1-yl)aniline

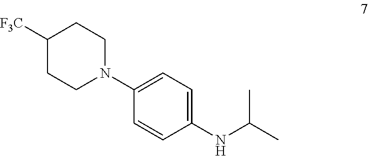 [SMILES: CC(C)Nc1ccc(N2CCC(C(F)(F)F)CC2)cc1]

The title compound 7 (12.0 mg) was prepared in a total yield of 12.6% as a yellow solid from 4-(4-(trifluoromethyl)piperidin-1-yl)aniline (81 mg, 0.3 mmol), acetone (0.2 mL), a drop of con. H₂SO₄, NaBH (AcO)₃ (76 mg, 0.36 mmol) and THF (10 mL) according to the procedure for 5. ¹H NMR (400 MHZ, DMSO-d₆) δ 6.78 (d, J=8.2 Hz, 2H), 6.54 (d, J=7.6 Hz, 2H), 3.52-3.41 (m, 3H), 2.62-2.52 (m, 2H), 2.43-2.35 (m, 1H), 1.86 (d, J=12.5 Hz, 2H), 1.61-1.50 (m, 2H), 1.09 (d, J=6.2 Hz, 6H). Mass (m/z): 287.2 [M+H]⁺.

### Compound 8

### N1,N1-dimethyl-N4-(4-(4-(trifluoromethyl)piperidin-1-yl)phenyl)cyclohexane-1,4-diamine

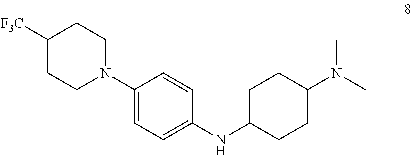 [SMILES: CN(C)C1CCC(Nc2ccc(N3CCC(C(F)(F)F)CC3)cc2)CC1]

The title compound 8 (53 mg) was prepared in a total yield of 19.4% as brown oil from 4-(4-(trifluoromethyl)piperidin-1-yl)aniline (180 mg, 0.74 mmol), 4-(dimethylamino)cyclohexan-1-one (125 mg, 0.89 mmol), acetic acid (3.7 mg, 0.061 mmol), sodium triacetoxyborohydride (469 mg, 2.22 mmol) and DCE (5 mL) according to the procedure for 5. ¹H NMR (300 MHz, CDCl₃) δ=6.84 (d, J=8.7 Hz, 2H), 6.64-6.50 (m, 2H), 3.51 (d, J=10.8 Hz, 2H), 3.13 (t, J=11.0 Hz, 1H), 2.58 (t, J=12.0 Hz, 2H), 2.30 (d, J=4.2 Hz, 6H), 2.24-2.14 (m, 2H), 1.95 (m, 4H), 1.89-1.73 (m, 4H), 1.62 (m, 3H), 1.45-1.28 (m, 1H). Mass (m/z): 185.7 [M+2H]²⁺/2.

### Compound 9

### 4-((4-(4-(trifluoromethyl)piperidin-1-yl)phenyl)amino)cyclohexane-1-carboxylic acid

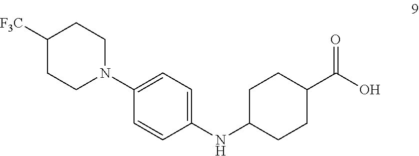 [SMILES: O=C(O)C1CCC(Nc2ccc(N3CCC(C(F)(F)F)CC3)cc2)CC1]

The title compound 9 (127 mg) was prepared in a yield of 41.8% as a rosy brown solid with 1:1 mixture by ¹H NMR from 4-(4-(trifluoromethyl)piperidin-1-yl)aniline (200 mg, 0.82 mmol), 4-oxocyclohexane-1-carboxylic acid (140 mg, 0.98 mmol) and NaBH (OAc); (347 mg, 1.64 mmol) according to the procedure for 4. ¹H NMR (400 MHZ, DMSO-d₆) δ 6.74 (d, J=8.1 Hz, 2H), 6.50 (s, 2H), 3.41 (d, J=11.7 Hz, 2H), 3.25 (s, 1H), 3.05 (s, 1H), 2.42-2.27 (m, 2H), 2.15 (tt, J=11.9, 3.5 Hz, 1H), 1.91 (dt, J=35.0, 14.3 Hz, 5H), 1.69-1.49 (m, 4H), 1.41 (qd, J=13.2, 3.2 Hz, 2H), 1.09 (q, J=12.0 Hz, 1H). Mass (m/z): 371.5 [M+H]⁺.

### Compound 10

### 4-((4-(4-(trifluoromethyl)piperidin-1-yl)phenyl)amino)cyclohexane-1-carboxamide

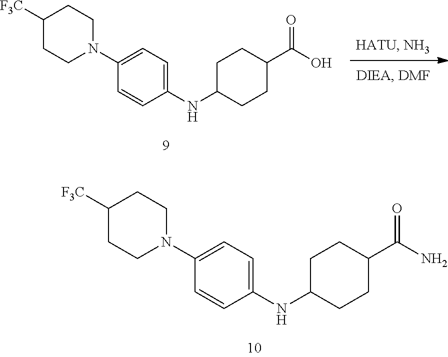

To a solution of 4-((4-(4-(trifluoromethyl)piperidin-1-yl)phenyl)amino)cyclohexane-1-carboxylic acid (70 mg, 0.19 mmol) and HATU (86 mg, 0.23 mmol) in super dry N,N-dimethylformamide (5 mL) was added N-ethyl-N-isopropylpropan-2-amine (94 mL, 0.57 mmol). The mixture was stirred for 30 mins and ammonium hydroxide (0.5 mL) was added dropwise at 0° C. slowly. The resulting solution was stirred overnight at room temperature. The reaction mixture was added into water (15 mL) drop by drop with stirring. The precipitate was filtered, and the filter cake was wash with water 3 times and dry in vacuo. The residue was purified by prep-TLC to give desired product 10 (28.3 mg) as pale peach powder with 1:1 mixture by ¹H NMR in a yield of 40.5%. ¹H NMR (400 MHZ, DMSO-d₆) δ 7.19 (s, 1H), 6.74 (d, J=8.3 Hz, 2H), 6.67 (s, 1H), 6.49 (d, J=8.2 Hz, 2H), 4.89 (s, 1H), 3.42 (d, J=11.8 Hz, 2H), 3.04 (s, 1H), 2.54 (d, J=11.2 Hz, 2H), 2.41-2.28 (m, 1H), 2.10-1.92 (m, 3H), 1.85 (d, J=12.7 Hz, 2H), 1.76 (d, J=13.1 Hz, 2H), 1.56 (td, J=13.9, 10.1 Hz, 2H), 1.50-1.36 (m, 2H), 1.06 (q, J=12.3 Hz, 2H). Mass (m/z): 370.3 [M+H]⁺.

### Compound 11

### N-(tert-butyl)-4-(4-(trifluoromethyl)piperidin-1-yl)aniline

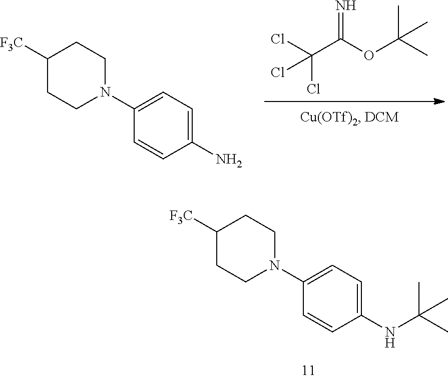 [SMILES: Nc1ccc(N2CCC(C(F)(F)F)CC2)cc1 ; CC(C)(C)OC(=N)C(Cl)(Cl)Cl ; CC(C)(C)Nc1ccc(N2CCC(C(F)(F)F)CC2)cc1]

To a solution of 4-(4-(trifluoromethyl)piperidin-1-yl)aniline (122 mg, 0.5 mmol) in dry DCM (20 mL) was added Cu(OTf)₂ (9.0 mg, 25 umol) and tert-butyl 2,2,2-trichloroacetimidate (272.5 mg, 1.25 mmol). Then the reaction was stirred at RT for 2 hr under argon. The reaction was washed with water (20 mL×3), dried over Na₂SO₄ and concentrated. The residue was purified by prep-TLC (EA/PE=1/2) to afford the desired product 11 (8.6 mg, 5.7%) as a yellow solid. ¹H NMR (400 MHZ, DMSO-d₆) δ 10.23 (s, 2H), 7.25-7.17 (m, 2H), 7.11-7.03 (m, 2H), 3.88 (d, J=12.6 Hz, 2H), 2.79 (t, J=11.6 Hz, 2H), 2.59-2.53 (m, 1H), 1.89 (d, J=12.0 Hz, 2H), 1.57-1.44 (m, 2H), 1.27 (s, 9H). Mass (m/z): 301.2 [M+H]⁺.

### Compound 12

### N-(4-methoxybutyl)-4-(4-(trifluoromethyl)piperidin-1-yl)aniline

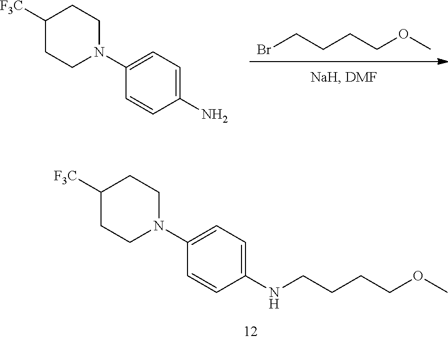

To a solution of 4-(4-(trifluoromethyl)piperidin-1-yl)aniline (100 mg, 0.4 mmol) in super dry N,N-dimethylformamide (5 mL), was added NaH (19.7 mg, 0.5 mmol) at 0° C. The mixture was stirred for 30 mins and 1-bromo-4-methoxybutane (68 mg, 0.4 mmol) was added. The resulting solution was stirred for overnight at 100° C. The reaction was diluted with water (20 mL) at 0° C. and extracted with ethyl acetate (5 mL) 3 times. The organic layers were combined and washed with water, sat. NH₄Cl (aq), brine respectively, dried over MgSO₄, filtered, and concentrated under reduced pressure. The residue was purified by prep-HPLC to give 28 mg of desired product 12 as pale rosy brown oil in a yield of 20.7%. ¹H NMR (400 MHZ, DMSO-d₆) δ 6.75 (d, J=8.8 Hz, 2H), 6.47 (d, J=8.8 Hz, 2H), 5.09 (s, 1H), 3.41 (d, J=12.0 Hz, 2H), 3.31 (d, J=6.0 Hz, 2H), 3.21 (s, 3H), 2.92 (s, 2H), 2.53 (dd, J=11.0, 2.0 Hz, 2H), 2.43-2.28 (m, 1H), 1.91-1.77 (m, 2H), 1.46-1.65 (m, 6H). Mass (m/z): 331.3 [M+H]⁺.

### Compound 13

### N-ethyl-4-(4-(trifluoromethyl)piperidin-1-yl)aniline

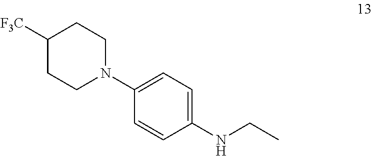 [SMILES: CCNc1ccc(N2CCC(C(F)(F)F)CC2)cc1]

The title compound 13 (32 mg) was prepared in a yield of 28.7% as a soil powder from 4-(4-(trifluoromethyl)piperidin-1-yl)aniline (100 mg, 0.4 mmol), iodoethane (32 mg, 0.4 mmol) and potassium carbonate (57 mg, 0.4 mmol) according to the procedure for 12. ¹H NMR (400 MHz, DMSO-d₆) δ 6.75 (d, J=8.4 Hz, 2H), 6.47 (d, J=8.3 Hz, 2H), 5.05 (s, 1H), 3.41 (d, J=11.9 Hz, 2H), 2.94 (q, J=7.1 Hz, 2H), 2.54 (s, 2H), 2.40-2.26 (m, 1H), 1.84 (d, J=12.4 Hz, 2H), 1.55 (qd, J=12.3, 4.1 Hz, 2H), 1.12 (t, J=7.1 Hz, 3H). Mass (m/z): 273.5 [M+H]⁺.

### Compound 14

### N-propyl-4-(4-(trifluoromethyl)piperidin-1-yl)aniline

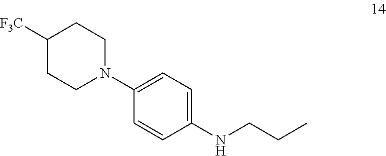 [SMILES: CCCNc1ccc(N2CCC(C(F)(F)F)CC2)cc1]

The title compound 14 (34.4 mg) was prepared in a yield of 58.7% as an off-white powder from 4-(4-(trifluoromethyl)piperidin-1-yl)aniline (50 mg, 0.2 mmol), 1-iodopropane (35 mg, 0.2 mmol) and potassium carbonate (42 mg, 0.3 mmol) according to the procedure for 12. ¹H NMR (400 MHZ, DMSO-d₆) δ 6.75 (d, J=8.8 Hz, 2H), 6.48 (d, J=8.8 Hz, 2H), 5.12 (s, 1H), 3.42 (d, J=12.1 Hz, 2H), 2.89 (t, J=7.1 Hz, 2H), 2.55 (s, 2H), 2.29-2.42 (m, 1H), 1.85 (d, J=12.1 Hz, 2H), 1.64-1.45 (m, 4H), 0.92 (t, J=7.4 Hz, 3H). Mass (m/z): 287.3 [M+H]⁺.

### Compound 15

### N-pentyl-4-(4-(trifluoromethyl)piperidin-1-yl)aniline

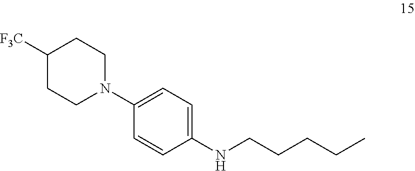 [SMILES: CCCCCNc1ccc(N2CCC(C(F)(F)F)CC2)cc1]

The title compound 15 (34.4 mg) was prepared in a yield of 51.4% as an off white powder from 4-(4-(trifluoromethyl)piperidin-1-yl)aniline (50 mg, 0.2 mmol), 1-iodopentane (40 mg, 0.2 mmol) and potassium carbonate (42 mg, 0.3 mmol) according to the procedure for 12. ¹H NMR (400 MHz, DMSO-d₆) δ 6.75 (d, J=8.8 Hz, 2H), 6.47 (d, J=8.8 Hz, 2H), 5.08 (s, 1H), 3.49-3.38 (m, 2H), 2.91 (t, J=7.1 Hz, 2H), 2.56-2.52 (m, 2H), 2.36 (dt, J=8.8, 4.4 Hz, 1H), 1.85 (d, J=12.2 Hz, 2H), 1.64-1.43 (m, 4H), 1.32 (tq, J=6.0, 2.8 Hz, 4H), 0.94-0.82 (m, 3H). Mass (m/z): 315.3 [M+H]⁺.

### Compound 16

### 1-methyl-N-(4-(4-(trifluoromethyl)piperidin-1-yl)phenyl)pyrrolidin-3-amine

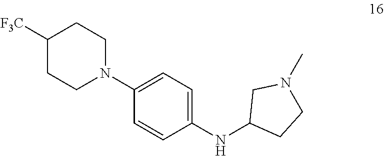 [SMILES: CN1CCC(Nc2ccc(N3CCC(C(F)(F)F)CC3)cc2)C1]

The title compound 16 (31.1 mg) was prepared in a yield of 23.2% as an off-white powder with 1:1 mixture by ¹H NMR from 4-(4-(trifluoromethyl)piperidin-1-yl)aniline (100 mg, 0.4 mmol), 1-methylpyrrolidin-3-one (48 mg, 0.49 mmol) and NaBH (OAc) 3 (173 mg, 0.82 mmol) according to the procedure for 4. ¹H NMR (400 MHZ, DMSO-d₆) δ 6.80 (d, J=8.9 Hz, 2H), 6.51 (d, J=8.9 Hz, 2H), 5.50 (s, 1H), 4.04 (s, 1H), 3.46 (d, J=12.2 Hz, 3H), 3.07 (d, J=46.4 Hz, 2H), 2.74 (s, 3H), 2.53 (d, J=2.4 Hz, 2H), 2.45-2.25 (m, 2H), 1.84 (d, J=3.7 Hz, 3H), 1.46-1.61 (m, 2H), 1.23 (d, J=3.3 Hz, 2H). Mass (m/z): 328.2 [M+H]⁺.

### Compound 17

### N-(4-(4-(trifluoromethyl)piperidin-1-yl)phenyl)tetrahydro-2H-pyran-4-amine

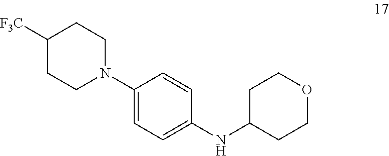 [SMILES: FC(F)(F)C1CCN(c2ccc(NC3CCOCC3)cc2)CC1]

The title compound 17 (23.9 mg) was prepared in a total yield of 35.8% as a purple solid with 1:2 mixture by ¹H NMR from 4-(4-(trifluoromethyl)cyclohexyl)aniline (50 mg, 0.205 mmol), tetrahydro-4H-pyran-4-one (25 mg, 0.246 mmol), AcOH (0.1 mL), NaBH (OAc) 3 (87 mg, 0.41 mmol) and DCE (10 mL) according to the procedure for 1-1. ¹H NMR (400 MHZ, DMSO-d₆) δ 7.29-6.83 (m, 4H), 3.94-3.83 (m, 2H), 3.79-3.66 (m, 2H), 3.53 (s, 1H), 3.30 (s, 2H), 3.06 (s, 1H), 2.62 (s, 1H), 1.96 (s, 2H), 1.80 (d, J=12.4 Hz, 2H), 1.74-1.38 (d, m, 4H). Mass (m/z): 329.3 [M+H]⁺.

### Compound 18

### (R)-2-oxo-N-((4-((4-(4-(trifluoromethyl)piperidin-1-yl)phenyl)amino)cyclohexyl)methyl)imidaz olidine-4-carboxamide

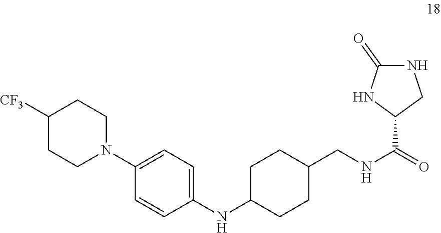 [SMILES: O=C1NC[C@H](C(=O)NCC2CCC(Nc3ccc(N4CCC(C(F)(F)F)CC4)cc3)CC2)N1]

The title compound 18 (6.1 mg) was prepared in a total yield of 27% as a rosy brown solid from N-(4-(aminomethyl)cyclohexyl)-4-(4-(trifluoromethyl)piperidin-1-yl)aniline (17 mg, 0.05 mmol) according to the procedure for compound 1. ¹H NMR (400 MHZ, DMSO-d₆) δ 9.14 (s, 1H), 7.90 (s, 1H), 6.79 (d, J=8.4 Hz, 2H), 6.53 (d, J=8.4 Hz, 2H), 6.30 (s, 1H), 5.32 (s, 1H), 3.68-3.46 (m, 3H), 3.25-2.99 (m, 3H), 2.95-2.87 (m, 2H), 2.64-2.55 (m, 2H), 2.21 (m, 1H), 2.05-1.87 (m, 4H), 1.75-1.50 (m, 4H), 1.45-1.31 (m, 3H), 1.15-0.95 (m, 2H). Mass (m/z): 468.3 [M+H]⁺.

### Compound 19

### N-((4-((4-(4-(trifluoromethyl)piperidin-1-yl)phenyl)amino)cyclohexyl)methyl)pyrrolidine-3-car boxamide

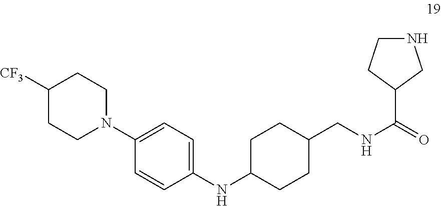 [SMILES: O=C(NCC1CCC(Nc2ccc(N3CCC(C(F)(F)F)CC3)cc2)CC1)C1CCNC1]

The title compound 19 (9.2 mg) was prepared in a total yield of 42% as a rosy brown solid from N-(4-(aminomethyl)cyclohexyl)-4-(4-(trifluoromethyl)piperidin-1-yl)aniline (17 mg, 0.05 mmol) according to the procedure for compound 1. ¹H NMR (400 MHZ, DMSO-d₆) δ 9.63 (s, 1H), 8.33 (s, 1H), 6.73 (d, J=8.4 Hz, 2H), 6.46 (d, J=8.4 Hz, 2H), 5.29 (s, 1H), 3.34-3.23 (m, 3H), 3.17-3.01 (m, 5H), 2.95-2.85 (m, 2H), 2.28-2.13 (m, 2H), 2.11 (m, 1H), 2.00-1.81 (m, 6H), 1.75-1.65 (m, 2H), 1.62-1.37 (m, 3H), 1.36-1.17 (m, 2H), 1.08-0.94 (m, 2H). Mass (m/z): 453.2 [M+H]⁺.

### Compound 20

### N-(4-(4-(trifluoromethyl)piperidin-1-yl)phenyl)piperidin-4-amine

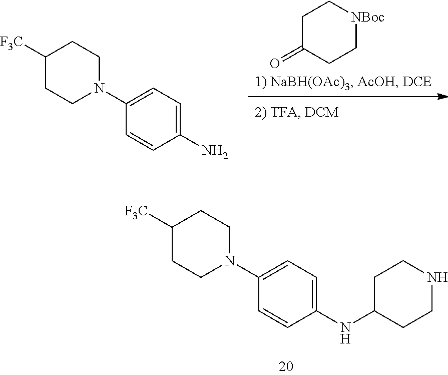 [SMILES: Nc1ccc(N2CCC(C(F)(F)F)CC2)cc1 ; CC(C)(C)OC(=O)N1CCC(=O)CC1 ; FC(F)(F)C1CCN(c2ccc(NC3CCNCC3)cc2)CC1]

To a solution of 4-(4-(trifluoromethyl)piperidin-1-yl)aniline (100 mg, 0.4 mmol) and tert-butyl 4-oxopiperidine-1-carboxylate (122.4 mg, 0.61 mmol) in 1,2-dichloroethane (5 mL) was added acetic acid (49 mg, 0.82 mmol). The mixture was stirred for 30 mins at room temperature before NaBH (OAc); (173 mg, 0.8 mmol) was added at 0° C. slowly. The resulting mixture was stirred for overnight. Dichloromethane (5 mL) was added and 2,2,2-trifluoroacetic acid (1 mL) was added. The resulting mixture was stirred for further 2 h. The reaction was quenched with saturated NaHCO₃ aqueous solution and extracted with dichloromethane (5 mL) for 3 times. The organic layers were combined and washed with water, sat. NaHCO₃, brine respectively, dried over MgSO₄, filtered, and concentrated under reduced pressure. The residue was purified by silica gel column chromatography (petroleum ether/AcOEt, 1/1) to give 37.2 mg of desired product 20 as a pale rosy brown powder in a yield of 28.3%. ¹H NMR (400 MHZ, DMSO-d₆) δ 9.08 (d, J=52.9 Hz, 2H), 7.59 (s, 1H), 6.75 (d, J=59.6 Hz, 3H), 3.46 (d, J=23.1 Hz, 3H), 3.27 (d, J=13.1 Hz, 2H), 2.95 (d, J=11.0 Hz, 2H), 2.01 (d, J=13.6 Hz, 3H), 1.88 (s, 2H), 1.60 (s, 4H), 1.24 (s, 2H). Mass (m/z): 328.6 [M+H]⁺

### Compound 21

### N-(4-(aminomethyl)cyclohexyl)-6-(4-(trifluoromethyl)piperidin-1-yl)pyridin-3-amine

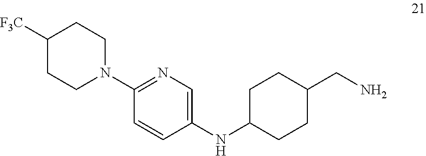 [SMILES: NCC1CCC(Nc2ccc(N3CCC(C(F)(F)F)CC3)nc2)CC1]

The title compound 21 (46.3 mg) was prepared in a yield of 32.87% as a pale yellow powder with 1:1 mixture by ¹H NMR from 6-(4-(trifluoromethyl)piperidin-1-yl)pyridin-3-amine (100 mg, 0.4 mmol), tert-butyl ((4-oxocyclohexyl)methyl)carbamate (111 mg, 0.49 mmol) and NaBH (OAc) 3 (173 mg, 0.8 mmol) according to the procedure for 20. ¹H NMR (400 MHZ, DMSO-d₆) δ 8.25 (s, 3H), 7.67 (s, 1H), 7.09 (s, 1H), 6.77 (d, J=9.0 Hz, 1H), 4.12 (d, J=12.7 Hz, 2H), 3.06 (s, 1H), 2.78-2.58 (m, 4H), 2.05-1.91 (m, 1H), 1.83 (d, J=12.5 Hz, 3H), 1.74 (s, 1H), 1.62 (s, 1H), 1.57-1.37 (m, 5H), 1.05 (td, J=25.1, 23.5, 10.3 Hz, 2H). Mass (m/z): 357.5 [M+H]⁺.

### Compound 22

### N-(4-(aminomethyl)cyclohexyl)-2-fluoro-4-methyl-6-(4-(trifluoromethyl)piperidin-1-yl)pyridin-3-amine

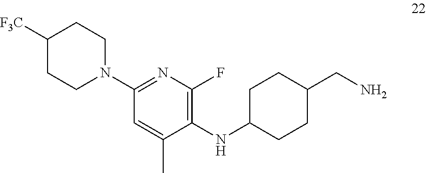 [SMILES: Cc1cc(N2CCC(C(F)(F)F)CC2)nc(F)c1NC1CCC(CN)CC1]

The title compound 22 (30.2 mg) was prepared in a yield of 42.56% as a pale rosy brown powder with 1:1 mixture by ¹H NMR from 2-fluoro-4-methyl-6-(4-(trifluoromethyl)piperidin-1-yl)pyridin-3-amine (50 mg, 0.18 mmol), tert-butyl ((4-oxocyclohexyl)methyl)carbamate (61 mg, 0.27 mmol) and NaBH (OAc); (76 mg, 0.36 mmol) according to the procedure for 20. ¹H NMR (400 MHZ, DMSO-d₆) δ 8.05 (s, 3H), 6.61 (s, 1H), 4.31-4.03 (m, 2H), 3.09 (s, 1H), 2.76 (d, J=12.9 Hz, 2H), 2.61 (s, 1H), 2.29 (s, 3H), 2.07-1.94 (m, 1H), 1.82 (t, J=16.8 Hz, 4H), 1.52 (s, 4H), 1.39 (qd, J=12.5, 4.0 Hz, 2H), 0.95 (q, J=10.3, 8.0 Hz, 1H). Mass (m/z): 389.4 [M+H]⁺.

### Compound 23

### N-(4-(aminomethyl)cyclohexyl)-3,5-difluoro-4-(4-methylcyclohexyl)aniline

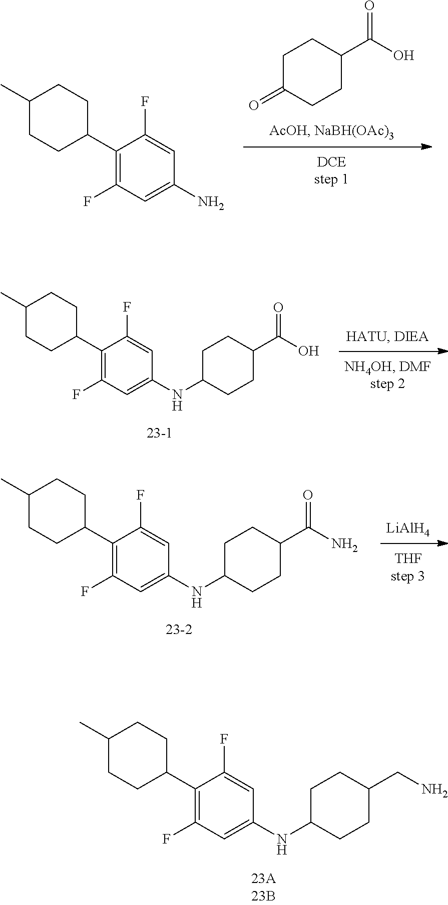

Step 1. Preparation of 4-((3,5-difluoro-4-(4-methylcyclohexyl)phenyl)amino)cyclohexane-1-carboxylic acid (23-1) The title compound 23-1 (605 mg) was prepared in a total yield of 77.7% as a brown solid from 3,5-difluoro-4-(4-methylcyclohexyl)aniline (500 mg, 2.212 mmol), 4-oxocyclohexane-1-carboxylic acid (377 mg, 2.655 mmol), AcOH (0.1 mL), NaBH (OAc) 3 (938 mg, 4.424 mmol) and DCE (15 mL) according to the procedure for 1-1. Mass (m/z): 353.3 [M+H]⁺.

Step 2. Preparation of 4-((3,5-difluoro-4-(4-methylcyclohexyl)phenyl)amino)cyclohexane-1-carboxamide (23-2) The title compound 23-2 (453 mg) was prepared in a total yield of 75.1% as a yellow solid from 4-((3,5-difluoro-4-(4-methylcyclohexyl)phenyl)amino)cyclohexane-1-carboxylic acid (605 mg, 1.72 mmol), NH₄OH (3 mL), HATU (770 mg, 2.23 mmol), DIEA (660 mg, 5.16 mmol) and DCE (15 mL) according to the procedure for 1. Mass (m/z): 352.3 [M+H]⁺.

Step 3. Preparation of N-(4-(aminomethyl)cyclohexyl)-3,5-difluoro-4-(4-methylcyclohexyl)aniline (23) To a solution of 4-((3,5-difluoro-4-(4-methylcyclohexyl)phenyl)amino)cyclohexane-1-carboxamide (50 mg, 0.142 mmol) in THF (5 mL), LiAlH₄ (21 mg, 0.57 mmol) was added at 0° C.. The mixture was stirred at room temperature for 16 h. The mixture was extracted by EA (25 mL×3). The combined organic layers were washed with brine (15 mL×3), dried over Na₂SO₄ and concentrated to give the crude product, which was purified by prep HPLC (XSelect-CSH-Prep 5 μm OBD, 19*150 mm column; ACN/water (0.5% TFA)=15%-25%-95%-95%-10%, 0 min-10 min-10.5 min-11.5 min-13 min) to give the desired product 23A (Rt=8.9 min) as a brown solid (2.4 mg, 5.0%) and 23B (Rt=9.8 min) as a brown solid (2.5 mg, 5.0%). 23A: 1H NMR (400 MHZ, DMSO-d₆) δ 6.15 (d, J=12.4 Hz, 2H), 3.08 (s, 1H), 2.93-2.87 (m, 3H), 2.72-2.63 (m, 2H), 1.97 (q, J=6.0, 4.0 Hz, 2H), 1.78 (d, J=7.2 Hz, 2H), 1.64-1.56 (m, 2H), 1.50 (s, 1H), 1.45-1.35 (m, 1H), 1.23 (q, J=7.2, 6.4 Hz, 3H), 1.12-0.99 (m, 4H), 0.93 (d, J=6.4 Hz, 3H). Mass (m/z): 338.3 [M+H]⁺. 23B: ¹H NMR (400 MHZ, DMSO-d₆) δ 6.16 (d, J=12.4 Hz, 2H), 3.36 (d, J=4.8 Hz, 1H), 2.93-2.80 (m, 4H), 2.68 (q, J=6.0 Hz, 2H), 1.65-1.46 (m, 9H), 1.41-1.28 (m, 3H), 1.19 (q, J=7.2, 5.6 Hz, 4H), 0.89 (d, J=6.4 Hz, 3H). Mass (m/z): 338.3 [M+H]⁺.

### Compound 24

### N-(4-(aminomethyl)cyclohexyl)-2-(4-isopropylpiperidin-1-yl)pyrimidin-5-amine

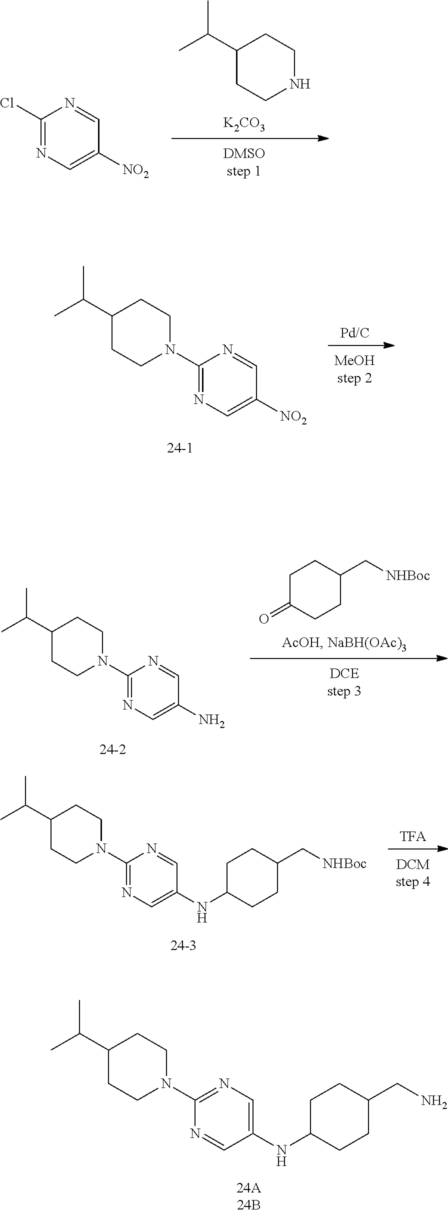

Step 1. Preparation of 2-(4-isopropylpiperidin-1-yl)-5-nitropyrimidine (24-1)

To a solution of 4-isopropylpiperidine (500 mg, 3.94 mmol), 2-chloro-5-nitropyrimidine (688 mg, 4.33 mmol), K₂CO₃ (815 mg, 5.91 mmol) in DMSO (20 mL) was stirred at 100° C. for 16 h. The mixture was extracted by EA (25 mL×3). The combined organic layers were washed with brine (15 mL×3), dried over Na₂SO₄ and concentrated to give the crude product, which was purified by TLC (EA: PE=1:5) to give the desired product as a yellow solid (752 mg, 76.3%). Mass (m/z): 251.3 [M+H]⁺.

Step 2. Preparation of 2-(4-isopropylpiperidin-1-yl)pyrimidin-5-amine (24-2) A solution of 2-(4-isopropylpiperidin-1-yl)-5-nitropyrimidine (752 mg, 3.42 mmol) and Pd/C (70 mg) in MeOH (20 mL) was stirred at rt for 2 h under H₂. The reaction was filtered and the filtrate was concentrated to give the desired product as a black solid (587 mg, 88%). Mass (m/z): 221.3 [M+H]⁺.

Step 3. Preparation of tert-butyl ((4-((2-(4-isopropylpiperidin-1-yl)pyrimidin-5-yl)amino)cyclohexyl)methyl)carbamate (24-3) The title compound 24-3 (32 mg) was prepared in a total yield of 31% as a yellow oil from 2-(4-isopropylpiperidin-1-yl)pyrimidin-5-amine (50 mg, 0.241 mmol), tert-butyl ((4-oxocyclohexyl)methyl)carbamate (66 mg, 0.289 mmol), AcOH (0.1 mL), NaBH (OAc) 3 (102 mg, 0.428 mmol) and DCE (10 mL) according to the procedure for 1-1. Mass (m/z): 432.3 [M+H]⁺.

Step 4. Preparation of N-(4-(aminomethyl)cyclohexyl)-2-(4-isopropylpiperidin-1-yl)pyrimidin-5-amine (24) To a solution of 24-3 (32 mg, 0.074 mmol) in DCM (10 mL) was added TFA (1 mL). The solution was stirred at room temperature for 1 h. The mixture was basified by 1M NaOH to pH=9 and concentrated, and then extracted by DCM (25 mL×3). The combined organic layers were washed with brine (15 mL×3), dried over Na₂SO₄ and concentrated, which was purified by Prep-HPLC (XSelect-CSH-Prep 5 μm OBD, 19*150 mm column; solvent system (ACN/water (0.5% TFA)=15%-24%-95%-95%-15%, 0-12 min-12.5 min-13.5 min-15 min) to give the desired product 24A (Rt=9.6 min) as a yellow solid (3.1 mg, 12.6%) and 24B (Rt=11.4 min) as yellow oil (5.1 mg, 6.3%). 24A: ¹H NMR (400 MHZ, DMSO-d₆) δ 7.65 (s, 2H), 4.48 (d, J=13.2 Hz, 1H), 2.63 (td, J=10.4, 8.4, 6.0 Hz, 3H), 1.96 (dd, J=14.4, 7.2 Hz, 3H), 1.69 (dd, J=50.8, 12.4 Hz, 3H), 1.51-1.32 (m, 2H), 1.20 (d, J=3.2 Hz, 9H), 1.10-0.95 (m, 4H), 0.84-0.80 (m, 6H). Mass (m/z): 332.3 [M+H]⁺. 24B: ¹H NMR (400 MHZ, DMSO-d₆) δ 7.69 (s, 2H), 4.48 (d, J=12.8 Hz, 1H), 3.36 (s, 4H), 2.75-2.58 (m, 4H), 1.69-1.33 (m, 11H), 1.20 (d, J=3.6 Hz, 4H), 1.10-0.98 (m, 2H), 0.83 (dd, J=6.8, 2.8 Hz, 6H). Mass (m/z): 332.3 [M+H]⁺.

### Compound 25

### N-(4-(aminomethyl)cyclohexyl)-2-methyl-6-(4-(trifluoromethyl)piperidin-1-yl)pyridin-3-amine

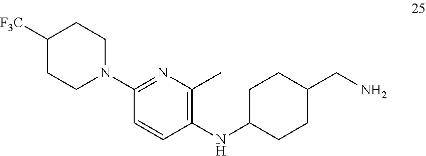 [SMILES: Cc1nc(N2CCC(C(F)(F)F)CC2)ccc1NC1CCC(CN)CC1]

The title compound 25 (7.9 mg) was prepared in a yield of 11.8% as a yellow solid with 1:1 mixture by ¹H NMR from 2-methyl-6-(4-(trifluoromethyl)piperidin-1-yl)pyridin-3-amine (50 mg, 0.19 mmol), tert-butyl ((4-oxocyclohexyl)methyl)carbamate (63 mg, 0.27 mmol) and NaBH (OAc) 3 (79 mg, 0.36 mmol) according to the procedure for 20. ¹H NMR (400 MHZ, DMSO-d₆) δ 8.09 (s, 3H), 6.91 (d, J=7.8 Hz, 1H), 6.56 (d, J=8.9 Hz, 1H), 4.12 (d, J=12.8 Hz, 2H), 3.04 (s, 1H), 2.75 (s, 1H), 2.70-2.55 (m, 3H), 2.25 (s, 1H), 2.21 (s, 2H), 2.05-1.91 (m, 2H), 1.83 (d, J=12.5 Hz, 3H), 1.55 (d, J=17.8 Hz, 3H), 1.44 (td, J=12.8, 12.4, 8.1 Hz, 3H), 1.05 (t, J=12.6 Hz, 2H), 0.88-0.81 (m, 1H). Mass (m/z): 371.5 [M+H]⁺.

### Compound 26

### N-(4-(aminomethyl)cyclohexyl)-2-methyl-6-(4-methylpiperidin-1-yl)pyridin-3-amine

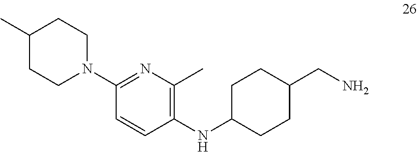 [SMILES: Cc1nc(N2CCC(C)CC2)ccc1NC1CCC(CN)CC1]

The title compound 26 (19.1 mg) was prepared in a yield of 24.8% as a pale yellow powder with 1:1 mixture by ¹H NMR from 2-methyl-6-(4-methylpiperidin-1-yl)pyridin-3-amine (50 mg, 0.24 mmol), tert-butyl ((4-oxocyclohexyl)methyl)carbamate (83 mg, 0.37 mmol) and NaBH (OAc) 3 (103 mg, 0.48 mmol) according to the procedure for 20. ¹H NMR (400 MHZ, DMSO-d₆) δ 11.07 (s, 1H), 8.16 (s, 3H), 6.68 (s, 1H), 4.21 (t, J=6.5 Hz, 1H), 2.68 (d, J=60.5 Hz, 4H), 2.37 (s, 3H), 2.04-1.91 (m, 1H), 1.82 (s, 2H), 1.73-1.42 (m, 9H), 1.06 (d, J=6.6 Hz, 3H), 0.91 (d, J=6.4 Hz, 4H). Mass (m/z): 317.4 [M+H]⁺. Compound 27

### 1-methyl-4-((4-(4-(trifluoromethyl)piperidin-1-yl)phenyl)amino)piperidin-2-one

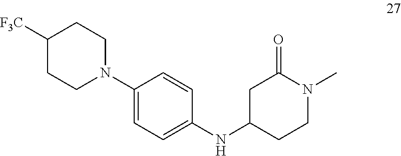 [SMILES: CN1CCC(Nc2ccc(N3CCC(C(F)(F)F)CC3)cc2)CC1=O]

The title compound 27 (89 mg) was prepared in a yield of 64.2% as a white solid from 1-methylpiperidine-2,4-dione (50 mg, 0.39 mmol), MeOH (5 mL) was added 4-(4-(trifluoromethyl)piperidin-1-yl)aniline (115 mg, 0.47 mmol), acetic acid (2.34 mg, 0.039 mmol) and sodium cyanoborohydride (73 mg, 1.17 mmol) according to the procedure for 5. ¹H NMR (300 MHz, CDCl₃) δ 6.86 (d, J=8.7 Hz, 2H), 6.59 (d, J=8.7 Hz, 2H), 3.75-3.72 (m, 1H), 3.53 (d, J=11.7 Hz, 2H), 3.39-3.32 (m, 2H), 2.97 (s, 3H), 2.85 (dd, J=18.0, 5.1, 1H), 2.59 (t, J=8.7 Hz, 2H), 2.32-2.23 (m, 1H), 2.21-2.14 (m, 1H), 2.12-2.08 (m, 1H), 1.96 (d, J=12.6 Hz, 2H), 1.86-1.71 (m, 3H). Mass (m/z): 356.2 [M+H]⁺.

### Compound 28

### N1-(2-(4-isopropylpiperidin-1-yl)pyrimidin-5-yl)cyclobutane-1,3-diamine

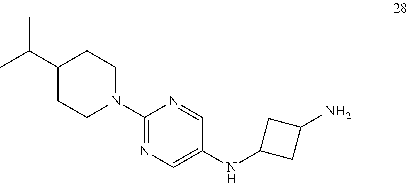 [SMILES: CC(C)C1CCN(c2ncc(NC3CC(N)C3)cn2)CC1]

The title compound 28 (22.3 mg) was prepared in a yield of 57.1% as a white solid with 1:4 mixture by ¹H NMR from tert-butyl (3-((2-(4-isopropylpiperidin-1-yl)pyrimidin-5-yl)amino)cyclobutyl)carbamate (53 mg, 0.14 mmol), DCM (1 mL) and TFA (1 mL) according to the procedure for 24. ¹H NMR (400 MHZ, DMSO-d₆) δ 7.77 (s, 0.4 H), 7.72 (s, 1.6 H), 5.33 (d, J=6.4 Hz, 0.8 H), 5.19 (d, J=7.2 Hz, 0.2 H), 4.45 (dt, J=12.6, 2.6 Hz, 2H), 3.80-3.70 (m, 1H), 3.56-3.49 (m, 1H), 3.32-3.20 (m, 2H), 2.62-2.55 (m, 2H), 2.50-2.48 (m, 0.8H), 2.08-1.95 (m, 3.2H), 1.66-1.56 (m, 2H), 1.52-1.42 (m, 0.2H), 1.39-1.30 (m, 0.8H), 1.21-1.12 (m, 1H), 1.08-0.96 (m, 2H), 0.82 (d, J=6.8 Hz, 6H). Mass (m/z): 290.3 [M+H]⁺.

### Compound 29

### N1-(2-(4-propylpiperidin-1-yl)pyrimidin-5-yl)cyclohexane-1,4-diamine

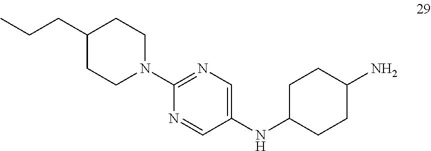 [SMILES: CCCC1CCN(c2ncc(NC3CCC(N)CC3)cn2)CC1]

The title compound 29 (32.2 mg) was prepared in a total yield of 32.3% as a yellow solid with 1:2 mixture by ¹H NMR from tert-butyl ((4-((4-(4-(trifluoromethyl)piperidin-1-yl)phenyl)amino)cyclohexyl)methyl)carbamate(130 mg, 0.312 mmol), TFA (1 mL) and DCM (10 mL) according to the procedure for 24. ¹H NMR (400 MHz, DMSO-d₆) δ 8.24 (s, 2H), 7.89 (d, J=14.4 Hz, 2H), 4.86 (dd, J=20.4, 7.2 Hz, 1H), 4.38 (dd, J=13.6, 3.2 Hz, 2H), 3.08-2.95 (m, 1H), 2.64 (tt, J=12.8, 2.4 Hz, 2H), 1.95 (tt, J=8.6, 3.2 Hz, 2H), 1.78-1.51 (m, 5H), 1.40 (ddd, J=15.2, 12.0, 6.8 Hz, 2H), 1.31-1.23 (m, 2H), 1.19-1.07 (m, 3H), 0.97 (qd, J=12.4, 4.0 Hz, 2H), 0.83 (t, J=7.2 Hz, 3H). Mass (m/z): 318.3 [M+H]⁺.

### Compound 30

### N-(4-(aminomethyl)cyclohexyl)-6-(4-butylpiperidin-1-yl)-2-methylpyridin-3-amine

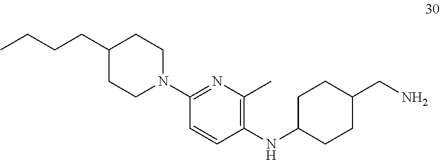 [SMILES: CCCCC1CCN(c2ccc(NC3CCC(CN)CC3)c(C)n2)CC1]

The title compound 30 (7.2 mg) was prepared in a yield of 10.1% as a pale yellow powder with 1:1 mixture by ¹H NMR from 6-(4-butylpiperidin-1-yl)-2-methylpyridin-3-amine (50 mg, 0.20 mmol), tert-butyl ((4-oxocyclohexyl)methyl)carbamate (73 mg, 0.32 mmol) and according to the procedure for 20. ¹H NMR (400 MHZ, DMSO-d₆) δ 7.73 (s, 3H), 7.43 (s, 1H), 2.79 (t, J=6.5 Hz, 2H), 2.67 (d, J=12.5 Hz, 1H), 2.39 (d, J=9.0 Hz, 3H), 1.98 (td, J=18.0, 16.5, 9.6 Hz, 2H), 1.85-1.73 (m, 4H), 1.66-1.40 (m, 8H), 1.28 (dd, J=7.8, 3.3 Hz, 6H), 0.86 (dt, J=10.8, 6.8 Hz, 4H). Mass (m/z): 331.3 [M+H]⁺.

### Compound 31

### N-(4-(aminomethyl)cyclohexyl)-6-(4-isopropylpiperidin-1-yl)-2-methylpyridin-3-amine

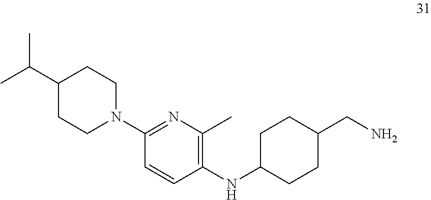 [SMILES: Cc1nc(N2CCC(C(C)C)CC2)ccc1NC1CCC(CN)CC1]

The title compound 31 (16.0 mg) was prepared in a yield of 20.6% as a pale yellow powder with 1:1 mixture by ¹H NMR from 6-(4-isopropylpiperidin-1-yl)-2-methylpyridin-3-amine (50 mg, 0.21 mmol), tert-butyl ((4-oxocyclohexyl)methyl)carbamate (73 mg, 0.32 mmol) and according to the procedure for 20. ¹H NMR (400 MHZ, DMSO-d₆) δ 8.26 (s, 3H), 6.96 (s, 1H), 6.59 (s, 1H), 4.07 (s, 2H), 2.75 (s, 1H), 2.61 (s, 2H), 2.28 (s, 3H), 1.96 (s, 1H), 1.83 (s, 2H), 1.77-1.35 (m, 9H), 1.20 (d, J=26.6 Hz, 4H), 1.02 (d, J=11.7 Hz, 1H), 0.88 (s, 3H), 0.86 (s, 3H). Mass (m/z): 345.6 [M+H]⁺.

### Compound 32

### N-(4-(aminomethyl)cyclohexyl)-6-(4,4-dimethylpiperidin-1-yl)-2-methylpyridin-3-amine

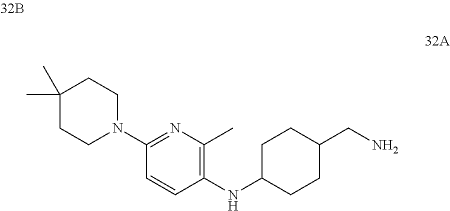 [SMILES: Cc1nc(N2CCC(C)(C)CC2)ccc1NC1CCC(CN)CC1]

The title compound 32 was prepared from 6-(4,4-dimethylpiperidin-1-yl)-2-methylpyridin-3-amine (50 mg, 0.23 mmol), tert-butyl ((4-oxocyclohexyl)methyl)carbamate (77 mg, 0.34 mmol) and according to the procedure for 20. The mixture was purified by preparative HPLC (Column: X Select-CSH-Prep 5 μm OBD, 19*150 mm; ACN/water (0.5% TFA)=5%-5%-95%-95%-5%, 0 min-1 min-10.0 min-11.0 min-15.0 min) afford compound 32A (Rt=3.69 min) as a yellow solid and compound 32B (Rt=3.79 min) as a yellow solid. 32A (18.2 mg, 23.9%): ¹H NMR (400 MHZ, DMSO-d₆) δ 8.15 (s, 3H), 6.99 (s, 1H), 6.68 (s, 1H), 3.37-3.41 (m, 3H), 2.75 (s, 1H), 2.61 (s, 1H), 2.32 (s, 3H), 1.82 (s, 2H), 1.55 (d, J=25.7 Hz, 6H), 1.35 (s, 4H), 1.22 (s, 1H), 1.00 (d, J=11.0 Hz, 1H), 0.94 (s, 6H). Mass (m/z): 331.5 [M+H]. 32B (8.8 mg, 11.6%): ¹H NMR (400 MHZ, DMSO-d₆) δ 8.04 (s, 3H), 7.22 (s, 1H), 6.66 (s, 1H), 3.52 (d, J=17.5 Hz, 2H), 2.75 (s, 1H), 2.63 (s, 1H), 2.27 (s, 2H), 1.99 (q, J=7.0 Hz, 3H), 1.80 (s, 2H), 1.48 (d, J=31.8 Hz, 7H), 1.35 (s, 4H), 0.94 (s, 6H), 0.88-0.81 (m, 1H). Mass (m/z): 331.5 [M+H]⁺.

Compound 33

### N1-(2-(3,3-dimethylazetidin-1-yl)pyrimidin-5-yl)cyclohexane-1,4-diamine

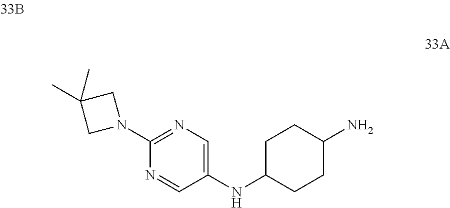 [SMILES: CC1(C)CN(c2ncc(NC3CCC(N)CC3)cn2)C1]

The title product 33A (Rt=6.8 min; 13.5 mg, 16.8%) as a yellow solid and 33B (Rt=8.0 min; 8.3 mg, 10.3%) as yellow solid prepared from a were tert-butyl (4-((2-(3,3-dimethylazetidin-1-yl)pyrimidin-5-yl)amino)cyclohexyl)carbamate (110 mg, 0.293 mmol), DCM (10 mL) and TFA (1 mL) according to the procedure for 24, which was purified by Prep-HPLC (XSelect-CSH-Prep 5 μm OBD, 19*150 mm column; solvent system (ACN/water (0.5% TFA)=15%-25%-95%-95%-15%, 0-13 min-13.5 min-14.5 min-16 min). 33A: ¹H NMR (400 MHZ, DMSO-d₆) δ 7.92-7.89 (m, 2H), 3.67-3.63 (m, 2H), 3.59 (d, J=7.6 Hz, 3H), 2.94 (dd, J=8.4, 2.4 Hz, 2H), 1.97-1.89 (m, 4H), 1.32 (td, J=8.4, 3.2 Hz, 3H), 1.25-1.21 (m, 6H), 1.19 (dd, J=9.2, 2.4 Hz, 3H). Mass (m/z): 276.3 [M+H]⁺. 33B: ¹H NMR (400 MHZ, DMSO-d₆) δ 7.92 (s, 2H), 3.64 (d, J=5.2 Hz, 3H), 3.22 (d, J=7.6 Hz, 2H), 1.73 (t, J=3.6 Hz, 8H), 1.26 (d, J=6.0 Hz, 6H), 1.22 (d, J=10.4 Hz, 3H). Mass (m/z): 276.3 [M+H]⁺.

### Compound 34

### N1-(2-(4-isopropylpiperidin-1-yl)pyrimidin-5-yl)cyclohexane-1,4-diamine

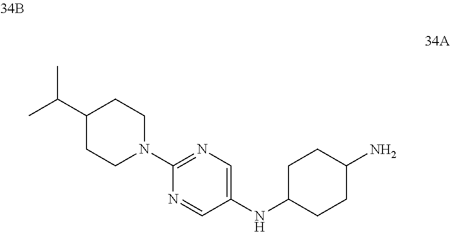 [SMILES: CC(C)C1CCN(c2ncc(NC3CCC(N)CC3)cn2)CC1]

The title product 34A (Rt=5.8 min) as a white solid (18.8 mg, 19.2%) and 34B (Rt=6.6 min) as a yellow solid (16.8 mg, 17.2%) were prepared from tert-butyl (4-((2-(4-isopropylpiperidin-1-yl)pyrimidin-5-yl)amino)cyclohexyl)carbamatee (130 mg, 0.312 mmol), DCM (10 mL) and TFA (1 mL) according to the procedure for 24 which was purified by Prep-HPLC (solvent system (ACN/water (0.5% TFA)=10%-45%-95%-95%-10%, 0-7 min-7.5 min-8.5 min-10 min). 34A: ¹H NMR (400 MHz, DMSO-d₆) δ 8.02 (s, 1H), 7.87 (s, 1H), 7.80 (d, J=4.8 Hz, 2H), 4.50 (d, J=12.8 Hz, 2H), 3.07 (d, J=11.2 Hz, 1H), 2.95 (s, 1H), 2.71-2.61 (m, 2H), 1.99-1.86 (m, 4H), 1.64 (d, J=12.4 Hz, 2H), 1.35 (dp, J=22.0, 7.6, 7.2 Hz, 3H), 1.20 (q, J=12.8, 10.0 Hz, 3H), 1.04 (qd, J=12.4, 4.0 Hz, 2H), 0.82 (d, J=6.8 Hz, 6H). Mass (m/z): 318.3 [M+H]⁺. 34B: ¹H NMR (400 MHZ, DMSO-d₆) δ 7.94 (s, 1H), 7.89 (s, 1H), 7.78 (s, 2H), 4.49-4.44 (m, 2H), 3.15 (d, J=39.6 Hz, 1H), 2.64 (td, J=12.8, 2.8 Hz, 2H), 1.74-1.54 (m, 10H), 1.37 (dq, J=13.2, 6.8 Hz, 1H), 1.23-1.17 (m, 2H), 1.04 (qd, J=12.4, 4.0 Hz, 2H), 0.85-0.80 (m, 6H). Mass (m/z): 318.3 [M+H]⁺.

### Compound 35

### 2-(4-isopropylpiperidin-1-yl)-N-(piperidin-4-yl)pyrimidin-5-amine

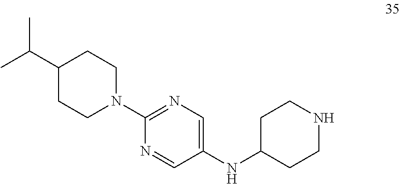 [SMILES: CC(C)C1CCN(c2ncc(NC3CCNCC3)cn2)CC1]

The title compound 35 (39.8 mg) was prepared in a total yield of 38.1% as yellow oil with 1:2 mixture 1H by NMR from tert-butyl 4-((2-(4-isopropylpiperidin-1-yl)pyrimidin-5-yl)amino)piperidine-1-carboxylate (140 mg, 0.347 mmol), TFA (1 mL), and DCM (10 mL) according to the procedure for 24. ¹H NMR (400 MHz, DMSO-d₆) δ 7.96 (s, 2H), 4.47 (dq, J=12.4, 2.8 Hz, 2H), 3.44-3.36 (m, 1H), 3.28 (t, J=15.2 Hz, 3H), 2.90 (p, J=15.2, 13.2 Hz, 3H), 2.64 (td, J=12.8, 2.8 Hz, 2H), 2.01-1.94 (m, 2H), 1.67-1.59 (m, 3H), 1.47 (dt, J=11.2, 3.2 Hz, 1H), 1.35 (dd, J=13.2, 6.4 Hz, 1H), 1.20 (d, J=4.0 Hz, 1H), 1.05 (tt, J=12.4, 6.4 Hz, 2H), 0.82 (d, J=6.8 Hz, 6H). Mass (m/z): 304.3 [M+H]⁺.

### Compound 36

### 2-(4-isopropylpiperidin-1-yl)-N-(pyrrolidin-3-yl)pyrimidin-5-amine

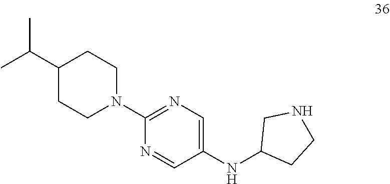 [SMILES: CC(C)C1CCN(c2ncc(NC3CCNC3)cn2)CC1]

The title compound 36 (35.0 mg) was prepared in a total yield of 36.5% as a yellow oil from tert-butyl 4-((2-(4-isopropylpiperidin-1-yl)pyrimidin-5-yl)amino)piperidine-1-carboxylate (130 mg, 0.334 mmol), TFA (1 mL), and DCM (10 mL) according to the procedure for 24. ¹H NMR (400 MHZ, DMSO-d₆) δ 7.91 (s, 2H), 4.47 (dt, J=14.0, 3.2 Hz, 2H), 3.97 (td, J=6.4, 3.2 Hz, 1H), 3.87 (s, 1H), 3.35 (dd, J=12.0, 6.0 Hz, 1H), 3.25-3.15 (m, 2H), 3.00 (dq, J=9.6, 4.8 Hz, 1H), 2.64 (td, J=12.8, 2.8 Hz, 2H), 2.23-2.14 (m, 1H), 1.66-1.58 (m, 3H), 1.37 (dq, J=13.2, 6.4 Hz, 2H), 1.04 (qd, J=12.4, 4.0 Hz, 3H), 0.83 (s, 3H), 0.81 (s, 3H). Mass (m/z): 290.3 [M+H]⁺.

### Compound 37

### N1-(4-(1H-pyrazol-1-yl)phenyl)cyclohexane-1,4-diamine

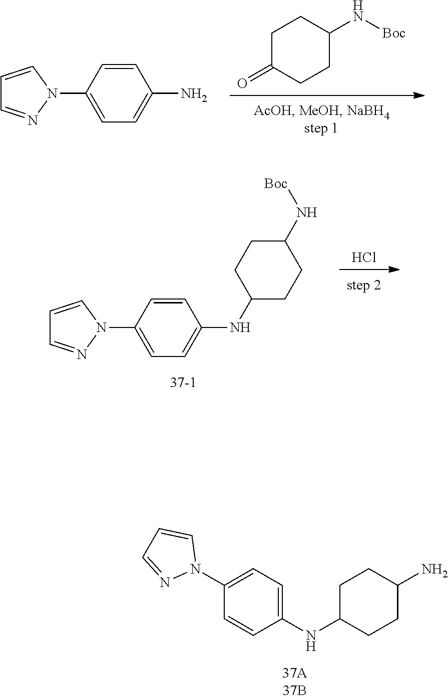 [SMILES: Nc1ccc(-n2cccn2)cc1 ; CC(C)(C)OC(=O)NC1CCC(=O)CC1 ; CC(C)(C)OC(=O)NC1CCC(Nc2ccc(-n3cccn3)cc2)CC1 ; Cl ; NC1CCC(Nc2ccc(-n3cccn3)cc2)CC1]

Step 1. Preparation of tert-butyl (4-((4-(1H-pyrazol-1-yl)phenyl)amino)cyclohexyl)carbamate (37-1)

To a solution of 4-(1H-pyrazol-1-yl)aniline (150 mg, 0.94 mmol) in MeOH (10 mL) were added a drop of AcOH and tert-butyl (4-oxocyclohexyl)carbamate (201.9 mg, 0.94 mmol), and the mixture was stirred at 50° C. for 1 h. Then NaBH₄ (71.24 mg, 1.88 mmol) was added after cooling to 25° C. Then the mixture was stirred at 25° C. for 16 hrs. LCMS showed the reaction was completed. The reaction was quenched with water (10 mL), extracted with EA (10 mL*3). The combined organic layers were washed with brine (20 mL), dried over sodium sulfate, filtered and concentrated. The residue was purified by combi-flash with EA/PE (1:3) to afford to give 37-1 (0.2 g, 59.3% yield) as a yellow solid. MS (m/z) 356.8 [M+H]⁺.

Step 2. Preparation of N-(4-(tert-butyl)phenyl)-N-(2,2,2-trifluoroethyl)cyclohexane-1,4-diamine (37) compound 37-1 (200 mg, 0.56 mmol) and HCl in 1,4-dioxane (10 mL, 4N) were placed in a flask stirred at 25° C. for 16 hrs. Excess 1,4-dioxane was distilled under vacuum and the residue was purified by prep-HPLC (column-Xbridge-C18 150×19 mm, 5 μm; Mobile phase: ACN-H₂O (0.1% FA), 2%-20%) to afford 37A (31.2 mg) as a white solid and 37B (21.9 mg) as a white solid. 37A: ¹H NMR (400 MHZ, DMSO-d₆) δ 8.42 (s, 1H), 8.20 (d, J=2.3 Hz, 1H), 7.61 (d, J=1.5 Hz, 1H), 7.47 (d, J=8.9 Hz, 2H), 6.64 (d, J=8.9 Hz, 2H), 6.45-6.42 (m, 1H), 5.61 (d, J=7.8 Hz, 1H), 3.17 (s, 1H), 2.89 (s, 1H), 1.97 (dd, J=34.9, 11.9 Hz, 4H), 1.39 (d, J=13.0 Hz, 2H), 1.23-1.14 (m, 2H). MS (m/z) 257.2 [M+H]⁺. HPLC: Rt: 2.643 min (Column: XBRIDGE 2.1*50 mm, 3.5 μm; Mobile Phase: H₂O (0.05% TFA)/ACN (0.05% TFA), ACN from 0% to 60% over 7 minutes, 7-8 min, ACN from 60% to 100%; 0.8 mL/min). 37B: 1H NMR (400 MHZ, CD₃OD) δ 8.55 (s, 1H), 7.96 (d, J=2.2 Hz, 1H), 7.63 (d, J=1.6 Hz, 1H), 7.40 (d, J=8.9 Hz, 2H), 6.73 (d, J=8.9 Hz, 2H), 6.45 (t, J=2.1 Hz, 1H), 3.60 (s, 1H), 3.23 (s, 1H), 1.97-1.74 (m, 8H). MS (m/z) 257.2 [M+H]⁺. HPLC: Rt: 3.112 min (Column: XBRIDGE 2.1*50 mm, 3.5 μm; Mobile Phase: H₂O (0.05% TFA) ACN (0.05% TFA), ACN from 0% to 60% over 7 minutes, 7-8 min, ACN from 60% to 100%; 0.8 mL/min).

### Compound 38

### N1-(2-(4-isopropylpiperidin-1-yl)pyrimidin-5-yl)cyclopentane-1,3-diamine

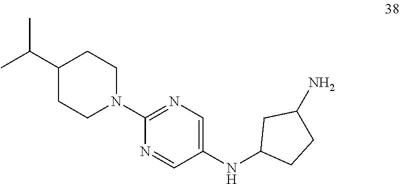 [SMILES: CC(C)C1CCN(c2ncc(NC3CCC(N)C3)cn2)CC1]

The title compound 38 (5.9 mg) was prepared in a total yield of 6.7% as a yellow solid from tert-butyl (3-((2-(4-isopropylpiperidin-1-yl)pyrimidin-5-yl)amino)cyclopentyl)carbamate (120 mg, 0.298 mmol), TFA (1 mL), and DCM (10 mL) according to the procedure for 24. ¹H NMR (400 MHz, DMSO-d₆) δ 7.86 (d, J=6.0 Hz, 2H), 4.46 (d, J=12.8 Hz, 1H), 3.81 (d, J=20.4 Hz, 2H), 3.61 (t, J=6.8 Hz, 2H), 2.62-2.60 (m, 1H), 2.11-2.02 (m, 1H), 1.99-1.88 (m, 3H), 1.62 (d, J=15.2 Hz, 3H), 1.57-1.48 (m, 1H), 1.36 (dt, J=11.6, 5.8 Hz, 2H), 1.04 (qd, J=12.4, 4.4 Hz, 2H), 0.83 (s, 3H), 0.81 (d, J=2.0 Hz, 3H). Mass (m/z): 304.3 [M+H]⁺.

### Compound 39

### N1-(3-(4-methoxybutoxy)-4-(4-(trifluoromethyl)piperidin-1-yl)phenyl)cyclohexane-1,4-diamine

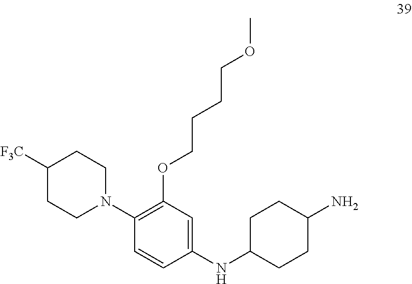 [SMILES: COCCCCOc1cc(NC2CCC(N)CC2)ccc1N1CCC(C(F)(F)F)CC1]

The title compound 39 (69.1 mg) was prepared in a total yield of 70.4% as a purple solid with 1:2 mixture by ¹H NMR from tert-butyl (4-((3-(4-methoxybutoxy)-4-(4-(trifluoromethyl)piperidin-1-yl)phenyl)amino)cyclohexyl) carba mate (120 mg, 0.221 mmol), TFA (1 mL), and DCM (10 mL) according to the procedure for 24. 1¹H NMR (400 MHZ, DMSO-d₆) δ 8.26 (s, 2H), 8.17 (s, 1H), 6.62 (d, J=8.4 Hz, 1H), 6.23 (d, J=24.8 Hz, 1H), 6.04 (s, 1H), 3.83 (d, J=7.6 Hz, 2H), 3.37-3.34 (m, 2H), 3.19 (s, 5H), 2.96 (d, J=51.2 Hz, 2H), 2.31 (s, 1H), 1.95 (d, J=11.6 Hz, 3H), 1.84-1.60 (m, 10H), 1.57-1.40 (m, 4H), 1.11 (d, J=12.0 Hz, 1H). Mass (m/z): 444.3 [M+H]⁺.

### Compound 40

### N1-(4-(4-(trifluoromethyl)piperidin-1-yl)phenyl)cyclohexane-1,4-diamine

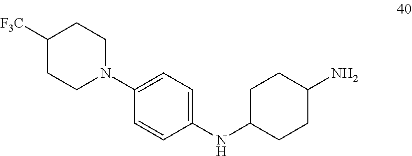 [SMILES: NC1CCC(Nc2ccc(N3CCC(C(F)(F)F)CC3)cc2)CC1]

The title compound 40 (56.2 mg) was prepared in a total yield of 48.9% as a purple solid with 1:2 mixture by ¹H NMR from N1-(4-(4-(trifluoromethyl)piperidin-1-yl)phenyl)cyclohexane-1,4-diamine (150 mg, 0.34 mmol), TFA (1 mL), and DCM (10 mL) according to the procedure for 24. ¹H NMR (400 MHZ, DMSO-d₆) δ 8.07 (s, 3H), 6.75-6.68 (m, 2H), 6.52-6.43 (m, 2H), 4.92 (d, J=7.2 Hz, 1H), 3.41-3.29 (m, 4H), 3.01 (s, 1H), 2.94-2.82 (m, 1H), 2.32 (d, J=9.2 Hz, 1H), 1.94 (d, J=12.0 Hz, 2H), 1.81 (d, J=12.0 Hz, 2H), 1.75-1.65 (m, 2H), 1.52 (qd, J=12.4, 4.1 Hz, 3H), 1.45-1.36 (m, 1H), 1.15-1.06 (m, 1H). Mass (m/z): 342.3 [M+H]⁺.

### Compound 41

### N1-(3-(2-aminoethoxy)-4-(4-(trifluoromethyl)piperidin-1-yl)phenyl)cyclohexane-1,4-diamine

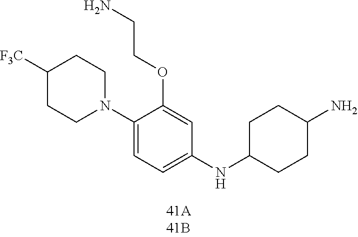 [SMILES: NCCOc1cc(NC2CCC(N)CC2)ccc1N1CCC(C(F)(F)F)CC1]

The title compound 41A, 41B was prepared from tert-butyl (2-(5-amino-2-(4-(trifluoromethyl)piperidin-1-yl)phenoxy)ethyl)carbamate (100 mg, 0.25 mmol), tert-butyl (4-oxocyclohexyl)carbamate (79 mg, 0.37 mmol) according to the procedure for compound 20. The residue was purified by preparative HPLC ((Column: X Select-CSH-Prep 5 μm OBD, 19*150 mm; ACN/water (0.5% TFA)=0%-30%-95%-95%-10%, 0 min-7 min-7.5 min-8.5 min-10.0 min) to afford compound 41A (Rt=4.39 min) in 5.6% yield as a white solid and 41B (Rt=4.82 min) in 6.24% yield as a white solid. 41A: ¹H NMR (400 MHz, DMSO-d₆) δ 8.01 (s, 3H), 7.86 (s, 3H), 6.56 (s, 1H), 6.17 (d, J=8.8 Hz, 1H), 3.13 (s, 2H), 2.01-1.85 (m, 8H), 1.87-1.46 (m, 9H), 1.45 (s, 2H), 0.85 (t, J=6.5 Hz, 2H). Mass (m/z): 401.4 [M+H]⁺. 41B: ¹H NMR (400 MHZ, DMSO-d₆) δ 8.05 (s, 3H), 7.80 (s, 3H), 6.37 (s, 1H), 6.28 (d, J=8.8 Hz, 1H), 4.17 (s, 1H), 3.13 (s, 2H), 2.07-1.88 (m, 6H), 1.79-1.56 (m, 11H), 1.45 (s, 1H), 0.85 (t, J=6.5 Hz, 2H). Mass (m/z): 401.4 [M+H]⁺.

### Compound 42

### N1-(2-(4-(trifluoromethyl)piperidin-1-yl)pyrimidin-5-yl)cyclohexane-1,4-diamine

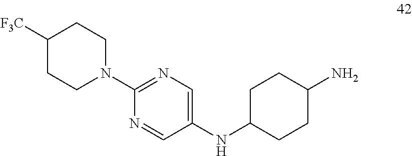 [SMILES: NC1CCC(Nc2cnc(N3CCC(C(F)(F)F)CC3)nc2)CC1]

The title compound 42 (102.7 mg) was prepared in a total yield of 87.9% as a yellow solid with 2:3 mixture by ¹H NMR from tert-butyl (4-((2-(4-(trifluoromethyl)piperidin-1-yl)pyrimidin-5-yl)amino)cyclohexyl)carbamate (150 mg, 0.39 mmol), TFA (1 mL), and DCM (10 mL) according to the procedure for 24. ¹H NMR (400 MHz, DMSO-d₆) δ 8.27 (s, 2H), 7.92 (d, J=13.6 Hz, 2H), 5.04 (d, J=6.0 Hz, 0.4H), 4.98 (d, J=8.4 Hz, 0.6H), 4.49 (d, J=13.2 Hz, 2H), 3.02 (dt, J=9.6, 4.8 Hz, 1H), 2.89 (tt, J=12.0, 3.6 Hz, 1H), 2.72 (td, J=12.8, 2.4 Hz, 2H), 1.95 (dt, J=12.4, 4.9 Hz, 2H), 1.80-1.74 (m, 2H), 1.70 (q, J=6.4, 5.2 Hz, 2H), 1.59-1.49 (m, 1H), 1.41 (dd, J=12.8, 3.2 Hz, 1H), 1.29 (tt, J=12.4, 6.4 Hz, 2H), 1.09 (qd, J=12.8, 11.6, 4.8 Hz, 1H). Mass (m/z): 344.3 [M+H]⁺.

### Compound 43

### N1-(2-(4-ethylpiperidin-1-yl)pyrimidin-5-yl)cyclohexane-1,4-diamine

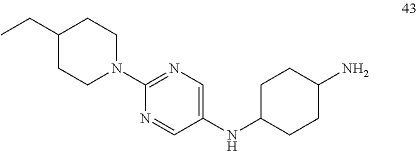 [SMILES: CCC1CCN(c2ncc(NC3CCC(N)CC3)cn2)CC1]

The title compound 43 (67.5 mg) was prepared in a total yield of 71.8% as a yellow solid with 1:1 mixture 1H by NMR from tert-butyl (4-((2-(4-(trifluoromethyl)piperidin-1-yl)pyrimidin-5-yl)amino)cyclohexyl)carbamate (150 mg, 0.39 mmol), TFA (1 mL), and DCM (10 mL) according to the procedure for 24. ¹H NMR (400 MHz, DMSO-d₆) δ 8.29 (s, 2H), 7.89 (d, J=14.4 Hz, 2H), 4.91 (d, J=6.0 Hz, 0.5H), 4.86 (d, J=8.4 Hz, 0.5H), 4.45-4.31 (m, 2H), 3.32 (d, J=21.6 Hz, 2H), 3.07-2.95 (m, 1H), 2.88 (dq, J=11.6, 6.0, 4.0 Hz, 1H), 2.63 (tt, J=12.4, 2.4 Hz, 2H), 1.94 (tt, J=9.6, 3.6 Hz, 2H), 1.73-1.59 (m, 4H), 1.57-1.48 (m, 1H), 1.46-1.36 (m, 1H), 1.28 (dp, J=11.2, 4.0 Hz, 1H), 1.18 (dd, J=8.4, 5.6 Hz, 2H), 0.96 (qd, J=12.4, 4.0 Hz, 2H), 0.82 (t, J=7.6 Hz, 3H). Mass (m/z): 304.3 [M+H]⁺.

### Compound 44

### N1-(2-(4-methylpiperidin-1-yl)pyrimidin-5-yl)cyclohexane-1,4-diamine

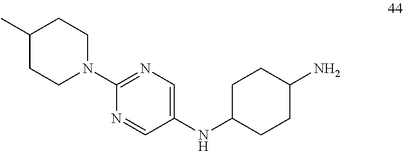 [SMILES: CC1CCN(c2ncc(NC3CCC(N)CC3)cn2)CC1]

The title compound 44 (17.0 mg) was prepared in a total yield of 26.2% as a yellow solid with 2:3 mixture ¹H NMR by from tert-butyl (4-((2-(4-(trifluoromethyl)piperidin-1-yl)pyrimidin-5-yl)amino)cyclohexyl)carbamate (87 mg, 0.224 mmol), TFA (1 mL), and DCM (10 mL) according to the procedure for 24. ¹H NMR (400 MHz, DMSO-d₆) δ 7.88 (d, J=14.0 Hz, 2H), 5.04 (d, J=6.0 Hz, 0.4H), 4.98 (d, J=8.4 Hz, 0.6H), 4.41-4.28 (m, 2H), 3.01 (dd, J=10.4, 5.6 Hz, 1H), 2.86 (t, J=12.0 Hz, 1H), 2.65 (td, J=12.4, 2.4 Hz, 2H), 1.93 (d, J=11.2 Hz, 2H), 1.69 (d, J=11.2 Hz, 2H), 1.55 (td, J=13.2, 3.6 Hz, 3H), 1.41-1.34 (m, 1H), 1.10 (t, J=11.6 Hz, 1H), 1.03-0.93 (m, 2H), 0.86 (d, J=6.4 Hz, 3H). Mass (m/z): 290.3 [M+H]⁺.

### Compound 45

### N1-(2-(4,4-dimethylpiperidin-1-yl)pyrimidin-5-yl)cyclohexane-1,4-diamine

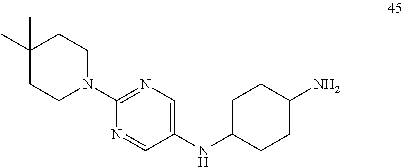 [SMILES: CC1(C)CCN(c2ncc(NC3CCC(N)CC3)cn2)CC1]

The title compound 45 (54.7 mg) was prepared in a total yield of 66.7% as a yellow solid with 1:1 mixture by ¹H NMR from tert-butyl (4-((2-(4,4-dimethylpiperidin-1-yl)pyrimidin-5-yl)amino)cyclohexyl)carbamate (109 mg, 0.270 mmol), TFA (1 mL), and DCM (10 mL) according to the procedure for 24. ¹H NMR (400 MHZ, DMSO-d₆) δ 7.94 (d, J=15.2 Hz, 2H), 4.92 (dd, J=19.2, 6.8 Hz, 1H), 3.55 (td, J=5.6, 2.0 Hz, 4H), 3.06 (d, J=8.8 Hz, 2H), 2.92 (td, J=11.6, 6.0 Hz, 1H), 2.03-1.96 (m, 2H), 1.83-1.65 (m, 3H), 1.58 (d, J=13.6 Hz, 1H), 1.49-1.42 (m, 1H), 1.32-1.26 (m, 4H), 1.19-1.08 (m, 1H), 0.94 (s, 6H). Mass (m/z): 304.3 [M+H]⁺.

### Compound 46

### N1-(2-(3-propylazetidin-1-yl)pyrimidin-5-yl)cyclohexane-1,4-diamine

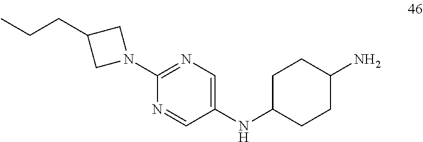 [SMILES: CCCC1CN(c2ncc(NC3CCC(N)CC3)cn2)C1]

The title compound 46 (4.3 mg) was prepared in a total yield of 11.1% as a yellow solid with 1:2 mixture by ¹H NMR from tert-butyl (4-((2-(3-propylazetidin-1-yl)pyrimidin-5-yl)amino)cyclohexyl)carbamate (52 mg, 0.134 mmol), TFA (1 mL), and DCM (10 mL) according to the procedure for 24. ¹H NMR (400 MHZ, DMSO-d₆) δ 7.91 (d, J=12.4 Hz, 2H), 4.97 (d, J=22.4 Hz, 1H), 3.96 (t, J=8.0 Hz, 2H), 3.49 (dd, J=8.0, 5.6 Hz, 2H), 3.04 (s, 1H), 2.93 (s, 1H), 1.99 (s, 2H), 1.73 (d, J=9.6 Hz, 2H), 1.57-1.52 (m, 2H), 1.47-1.40 (m, 2H), 1.32-1.19 (m, 5H), 1.16-1.10 (m, 1H), 0.88 (t, J=7.2 Hz, 3H). Mass (m/z): 290.3 [M+H]⁺.

### Compound 47

### N1-(2-(2-azaspiro[3.3]heptan-2-yl)pyrimidin-5-yl)cyclohexane-1,4-diamine

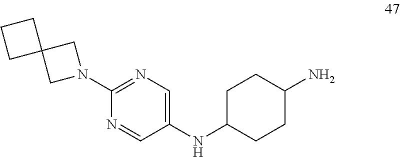 [SMILES: NC1CCC(Nc2cnc(N3CC4(CCC4)C3)nc2)CC1]

The title compound 47 (26.1 mg) was prepared in a total yield of 35.6% as a yellow solid with 1:2 mixture by NMR 1H from tert-butyl (4-((2-(2-azaspiro[3.3]heptan-2-yl)pyrimidin-5-yl)amino)cyclohexyl)carbamate (98 mg, 0.253 mmol), TFA (1 mL), and DCM (10 mL) according to the procedure for 24. ¹H NMR (400 MHZ, DMSO-d₆) δ 7.91 (d, J=13.2 Hz, 2H), 5.04 (d, J=6.0 Hz, 0.3H), 4.98 (d, J=8.4 Hz, 0.7H), 3.84 (s, 3H), 2.96 (dd, J=33.6, 21.6 Hz, 2H), 2.13 (t, J=7.6 Hz, 4H), 2.03-1.93 (m, 3H), 1.84-1.71 (m, 4H), 1.57-1.38 (m, 2H), 1.16-1.10 (m, 1H). Mass (m/z): 288.3 [M+H]⁺.

### Compound 48

### N-(3-(aminomethyl)cyclopentyl)-2-(4-isopropylpiperidin-1-yl)pyrimidin-5-amine

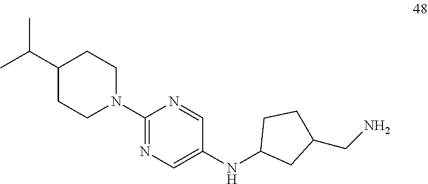 [SMILES: CC(C)C1CCN(c2ncc(NC3CCC(CN)C3)cn2)CC1]

The title compound 48 (6.1 mg) was prepared in a yield of 8.4% as a white powder with 1:1 mixture by ¹H NMR from 2-(4-isopropylpiperidin-1-yl)pyrimidin-5-amine (50 mg, 0.23 mmol), tert-butyl ((3-oxocyclopentyl)methyl)carbamate (73 mg, 0.34 mmol) and according to the procedure for 20. ¹H NMR (400 MHZ, DMSO-d₆) δ 7.99 (s, 3H), 7.86 (d, J=2.8 Hz, 2H), 4.51 (q, J=16.0, 14.9 Hz, 2H), 2.66 (d, J=23.1 Hz, 2H), 1.64 (d, J=12.9 Hz, 4H), 1.40 (q, J=6.6 Hz, 3H), 1.34-1.15 (m, 5H), 1.09 (td, J=12.5, 4.0 Hz, 3H), 0.86 (d, J=2.8 Hz, 4H), 0.84 (s, 3H). Mass (m/z): 317.3 [M+H]⁺.

### Compound 49

### N-(3-(aminomethyl)cyclobutyl)-2-(4-isopropylpiperidin-1-yl)pyrimidin-5-amine

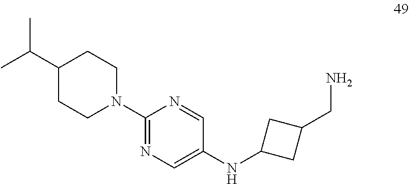 [SMILES: CC(C)C1CCN(c2ncc(NC3CC(CN)C3)cn2)CC1]

The title compound 49 (26.6 mg) was prepared in a three-step overall yield of 9.6% as a white powder with 1:1 mixture by ¹H NMR from 2-(4-isopropylpiperidin-1-yl)pyrimidin-5-amine (200 mg, 0.91 mmol), 3-oxocyclobutane-1-carboxylic acid (155 mg, 1.36 mmol) according to the procedure for 23. ¹H NMR (400 MHz, DMSO-d₆) δ 11.07 (s, 1H), 8.16 (s, 3H), 6.68 (s, 1H), 4.21 (t, J=6.5 Hz, 2H), 2.68 (d, J=60.5 Hz, 3H), 2.37 (s, 3H), 2.03-1.89 (m, 1H), 1.82 (s, 2H), 1.73-1.39 (m, 7H), 1.22 (d, J=3.1 Hz, 3H), 1.06 (d, J=6.6 Hz, 2H), 0.91 (d, J=6.4 Hz, 3H). Mass (m/z): 304.6 [M+H]⁺.

### Compound 50

### N1-(2-(4-isopropylpiperidin-1-yl)pyrimidin-5-yl)-4-methylcyclohexane-1,4-diamine

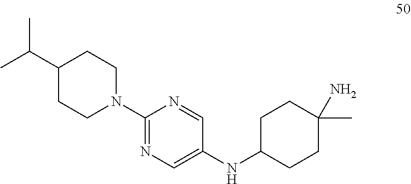 [SMILES: CC(C)C1CCN(c2ncc(NC3CCC(C)(N)CC3)cn2)CC1]

The title compound 50 (56.3 mg) was prepared in a yield of 53.4% as a pale yellow powder with 1:1 mixture by ¹H NMR from 2-(4-isopropylpiperidin-1-yl)pyrimidin-5-amine (70 mg, 0.34 mmol), tert-butyl (1-methyl-4-oxocyclohexyl)carbamate (110 mg, 0.52 mmol) and according to the procedure for 20. ¹H NMR (400 MHZ, DMSO-d₆) δ 8.10 (d, J=18.4 Hz, 3H), 7.92 (d, J=11.0 Hz, 2H), 4.82 (dd, J=36.7, 7.5 Hz, 1H), 4.56-4.42 (m, 2H), 3.13 (d, J=43.4 Hz, 1H), 2.62 (td, J=12.8, 2.5 Hz, 2H), 1.97-1.81 (m, 2H), 1.74 (d, J=13.4 Hz, 2H), 1.69-1.57 (m, 4H), 1.53 (h, J=7.5, 6.3 Hz, 1H), 1.40 (h, J=6.7 Hz, 1H), 1.27 (d, J=7.0 Hz, 4H), 1.07 (qd, J=12.3, 4.2 Hz, 2H), 0.85 (d, J=6.7 Hz, 6H). Mass (m/z): 332.4 [M+H]⁺.

### Compound 50

### 1-methyl-N4-(4-(4-(trifluoromethyl)piperidin-1-yl)phenyl)cyclohexane-1,4-diamine

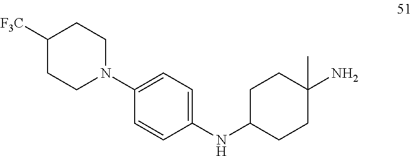 [SMILES: CC1(N)CCC(Nc2ccc(N3CCC(C(F)(F)F)CC3)cc2)CC1]

The title compound 51 (47.2 mg) was prepared in a total yield of 75.8% as a purple solid from tert-butyl (1-methyl-4-((4-(4-(trifluoromethyl)piperidin-1-yl)phenyl)amino)cyclohexyl)carbamate (79 mg, 0.174 mmol), TFA (1 mL), and DCM (10 mL) according to the procedure for 24. ¹H NMR (400 MHz, DMSO-d₆) δ 8.33 (d, J=36.4 Hz, 3H), 6.78 (s, 2H), 6.68-6.39 (m, 2H), 3.16 (d, J=55.2 Hz, 1H), 2.38 (s, 1H), 2.05-1.43 (m, 11H), 1.30 (d, J=7.5 Hz, 3H). Mass (m/z): 356.3 [M+H]⁺.

### Compound 52

### N1-(5-(4-(trifluoromethyl)piperidin-1-yl)pyrazin-2-yl)cyclohexane-1,4-diamine

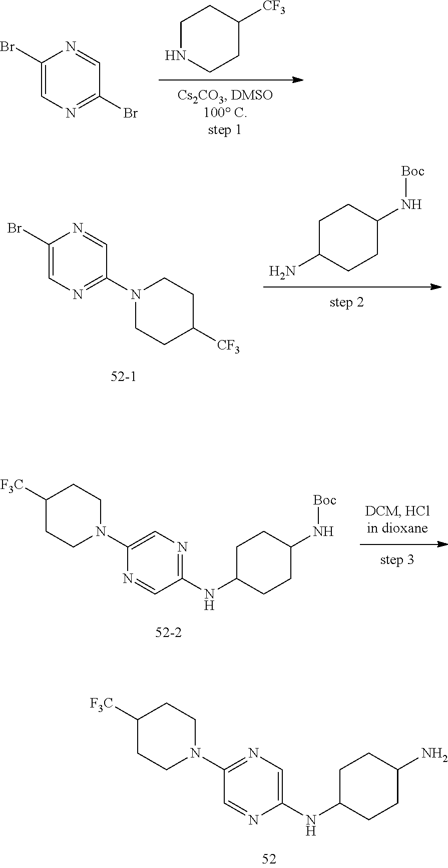 [SMILES: Brc1cnc(Br)cn1 ; FC(F)(F)C1CCNCC1 ; FC(F)(F)C1CCN(c2cnc(Br)cn2)CC1 ; CC(C)(C)OC(=O)NC1CCC(N)CC1 ; CC(C)(C)OC(=O)NC1CCC(Nc2cnc(N3CCC(C(F)(F)F)CC3)cn2)CC1 ; NC1CCC(Nc2cnc(N3CCC(C(F)(F)F)CC3)cn2)CC1]

Step 1. Preparation of 2-bromo-5-(4-(trifluoromethyl)piperidin-1-yl)pyrazine (52-1)

To a solution of 2,5-dibromopyrazine (3 g, 12.6 mmol) in DMSO (30 mL) was added 4-(trifluoromethyl)piperidine (1.93 g, 12.6 mmol) and Cs₂CO₃ (6.16 g, 18.9 mmol). Then the mixture was stirred at 100° C. for 2 hrs. LCMS showed the reaction was completed. The mixture was added into H₂O and filtered to give compound 52-1 (3.5 g, 89.7% yield) as a yellow solid. MS (m/z): 310.0 [M+H]⁺.

Step 2. Preparation of tert-butyl (4-((5-(4-(trifluoromethyl)piperidin-1-yl)pyrazin-2-yl)amino)cyclohexyl)carbamate (52-2)

To a solution of 2-bromo-5-(4-(trifluoromethyl)piperidin-1-yl)pyrazine (0.2 g, 0.64 mmol), tert-butyl (4-aminocyclohexyl)carbamate (139 mg, 0.64 mmol), Cs₂CO₃ (420 mg, 1.29 mmol) and Ruphos (60 mg, 0.13 mmol) in dioxane (10 mL) was added Pd₂(dba)₃ (59 mg, 0.064 mmol) at 25° C. under N₂. Then the mixture was stirred at 90° C. overnight. LCMS showed the reaction was completed. The reaction was filtered and concentrated. The residue was purified by combi-flash with EA/PE (1:2) to afford 52-2 (0.1 g, 34.8% yield) as a yellow solid. MS (m/z) 443.7 [M+H]⁺.

Step 3. Preparation of N1-(5-(4-(trifluoromethyl)piperidin-1-yl)pyrazin-2-yl) cycl ohexane-1,4-diamine (52)

To a solution of compound 3 (100 mg, 0.22 mmol) in THF (2 mL) was added HCl in dioxane (2 mL). Then the mixture was stirred at 25° C. for 1 h. LCMS showed the reaction was completed. The reaction was concentrated. The residue was purified by prep-HPLC to give 52 (6 mg, 7.7% yield) as a yellow solid. ¹H NMR (300 MHz, CD₃OD) δ 7.64 (d, J=1.3 Hz, 1H), 7.62 (s, 1H), 3.96 (d, J=12.6 Hz, 2H), 3.80 (s, 1H), 3.22-3.15 (m, 1H), 2.67 (t, J=12.6 Hz, 2H), 2.36-2.21 (m, 1H), 1.94-1.54 (m, 12H). MS (m/z) 344.2 [M+H]⁺.

### Compound 53

### N1-(4-(4-methylpiperidin-1-yl)-3-(trifluoromethyl)phenyl)cyclohexane-1,4-diamine

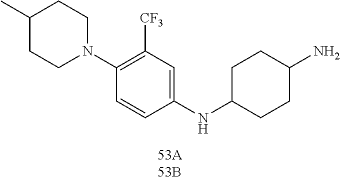 [SMILES: CC1CCN(c2ccc(NC3CCC(N)CC3)cc2C(F)(F)F)CC1]

The desired product 53A (Rt=10.9 min) as a yellow solid (15.1 mg, 16.1%) and 53B (Rt=14.4 min) as yellow oil (14.3 mg, 15.5%) were prepared from tert-butyl (4-((4-(4-methylpiperidin-1-yl)-3-(trifluoromethyl)phenyl)amino)cyclohexyl)carbamate (121 mg, 0.266 mmol), DCM (10 mL) and TFA (1 mL) according to the procedure for 24, which were purified by Prep-HPLC (Column: X Select-CSH-Prep 5 μm OBD, 19*150 mm; solvent system (ACN/water (0.5% TFA)=10%-90%-95%-95%-10%, 0-12 min-12.5 min-13.5 min-15 min). 53A: ¹H NMR (400 MHZ, DMSO-d₆) δ 7.20 (d, J=8.4 Hz, 1H), 6.70 (d, J=10.0 Hz, 2H), 5.63 (d, J=8.0 Hz, 1H), 3.10-3.00 (m, 1H), 2.71 (dt, J=11.6, 3.3 Hz, 2H), 2.58 (td, J=11.6, 2.4 Hz, 2H), 1.91-1.81 (m, 2H), 1.77-1.66 (m, 2H), 1.58 (dd, J=12.8, 3.5 Hz, 2H), 1.43-1.34 (m, 1H), 1.23-1.13 (m, 3H), 1.08 (t, J=9.2 Hz, 3H), 0.89 (d, J=6.4 Hz, 3H). Mass (m/z): 356.3 [M+H]⁺. 53B: ¹H NMR (400 MHZ, DMSO-d₆) δ 7.20 (d, J=8.4 Hz, 1H), 6.70 (d, J=10.0 Hz, 2H), 5.63 (d, J=8.0 Hz, 1H), 3.10-3.00 (m, 1H), 2.71 (dt, J=11.6, 3.3 Hz, 2H), 2.58 (td, J=11.6, 2.4 Hz, 2H), 1.91-1.81 (m, 2H), 1.77-1.66 (m, 2H), 1.58 (dd, J=12.8, 3.5 Hz, 2H), 1.43-1.34 (m, 1H), 1.23-1.13 (m, 3H), 1.08 (t, J=9.2 Hz, 3H), 0.89 (d, J=6.4 Hz, 3H). Mass (m/z): 356.3 [M+H]⁺.

### Compound 54

### N1-(3,5-difluoro-4-(4-(trifluoromethyl)piperidin-1-yl)phenyl)cyclohexane-1,4-diamine

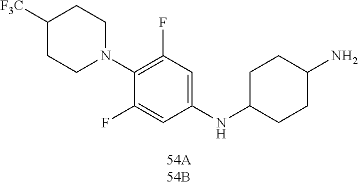 [SMILES: NC1CCC(Nc2cc(F)c(N3CCC(C(F)(F)F)CC3)c(F)c2)CC1]

The desired product 54A (Rt=12.2 min) as a white solid (44.4 mg, 37.0%) and 54B (Rt=12.9 min) as a yellow solid (42.0 mg, 34.8%) were prepared from tert-butyl (4-((3,5-difluoro-4-(4-(trifluoromethyl)piperidin-1-yl)phenyl)amino)cyclohexyl)carbamate (150 mg, 0.314 mmol), DCM (10 mL), and TFA (1 mL) according to the procedure for 24, which were purified by Prep-HPLC (Column: X Select-CSH-Prep 5 μm OBD, 19*150 mm; solvent system (ACN/water (0.5% TFA)=10%-85%-95%-95%-10%, 0-14 min-14.5 min-15.5 min-17 min). 54A: ¹H NMR (400 MHZ, DMSO-d₆) δ 7.80 (d, J=5.2 Hz, 3H), 6.17 (d, J=12.0 Hz, 2H), 3.05 (dq, J=11.2, 6.0, 3.6 Hz, 1H), 3.00-2.89 (m, 5H), 2.33 (dq, J=8.8, 5.2, 4.4 Hz, 1H), 1.99-1.84 (m, 4H), 1.82-1.73 (m, 2H), 1.54-1.33 (m, 4H), 1.11 (dt, J=13.2, 10.4 Hz, 2H). Mass (m/z): 378.3 [M+H]⁺. 54B: ¹H NMR (400 MHZ, DMSO-d₆) δ 7.80 (d, J=5.2 Hz, 3H), 6.17 (d, J=12.0 Hz, 2H), 3.05 (dq, J=11.2, 6.0, 3.6 Hz, 1H), 3.00-2.89 (m, 5H), 2.33 (dq, J=8.8, 5.2, 4.4 Hz, 1H), 1.99-1.84 (m, 4H), 1.82-1.73 (m, 2H), 1.54-1.33 (m, 4H), 1.11 (dt, J=13.2, 10.4 Hz, 2H). Mass (m/z): 378.3 [M+H]⁺.

### Compound 55

### N1-(4-(4,4-dimethylpiperidin-1-yl)-3-(trifluoromethyl)phenyl)cyclohexane-1,4-diamine

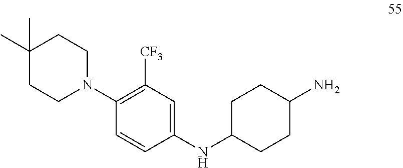 [SMILES: CC1(C)CCN(c2ccc(NC3CCC(N)CC3)cc2C(F)(F)F)CC1]

The title compound 55 (38.9 mg) was prepared in a total yield of 53.4% as a brown solid with 1:2 mixture by NMR from tert-butyl 1H (4-((4-(4,4-dimethylpiperidin-1-yl)-3-(trifluoromethyl)phenyl)amino)cyclohexyl)carbamate (93 mg, 0.198 mmol), TFA (1 mL), and DCM (10 mL) according to the procedure for 24. ¹H NMR (400 MHz, DMSO-d₆) δ 7.30 (dd, J=9.2, 6.8 Hz, 1H), 6.85-6.69 (m, 2H), 5.79 (dd, J=12.8, 6.8 Hz, 1H), 3.40-3.32 (m, 2H), 3.06 (ddd, J=19.6, 9.6, 4.4 Hz, 1H), 2.91 (tt, J=11.6, 3.6 Hz, 1H), 2.68-2.57 (m, 4H), 1.99-1.91 (m, 2H), 1.71 (dd, J=8.0, 4.0 Hz, 2H), 1.60-1.51 (m, 1H), 1.49-1.41 (m, 1H), 1.34 (t, J=5.6 Hz, 4H), 1.16-1.10 (m, 1H), 0.92 (s, 6H). Mass (m/z): 370.3 [M+H]⁺.

### Compound 56

### N1-(5-chloro-2-methyl-4-(4-(trifluoromethyl)piperidin-1-yl)phenyl)cyclohexane-1,4-diamine

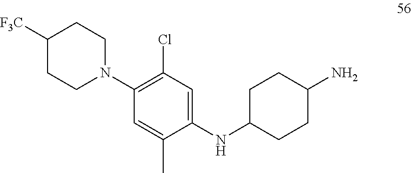 [SMILES: Cc1cc(N2CCC(C(F)(F)F)CC2)c(Cl)cc1NC1CCC(N)CC1]

The title compound 56 (13.9 mg) was prepared in a total yield of 20% as a brown solid with 1:2 mixture by ¹H NMR from tert-butyl (4-((4-(4,4-dimethylpiperidin-1-yl)-3-(trifluoromethyl)phenyl)amino)cyclohexyl)carbamate (88 mg, 0.18 mmol), TFA (1 mL), and DCM (10 mL) according to the procedure for 24. ¹H NMR (400 MHZ, DMSO-d₆) δ 6.82 (d, J=14.8 Hz, 1H), 6.53 (d, J=4.0 Hz, 1H), 4.34 (d, J=8.0 Hz, 1H), 3.08 (d, J=12.0 Hz, 3H), 2.61-2.52 (m, 3H), 2.09 (s, 1H), 2.00 (s, 2H), 1.98-1.91 (m, 3H), 1.87-1.76 (m, 3H), 1.69 (d, J=6.0 Hz, 1H), 1.56 (td, J=12.4, 4.0 Hz, 3H), 1.47-1.33 (m, 2H), 1.24 (d, J=13.2 Hz, 1H). Mass (m/z): 390.3 [M+H]⁺.

### Compound 57

### N1-(3-fluoro-2-methyl-4-(4-(trifluoromethyl)piperidin-1-yl)phenyl)cyclohexane-1,4-diamine

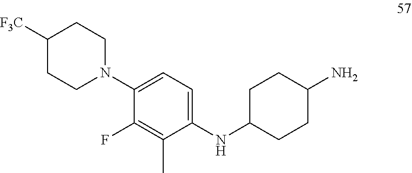 [SMILES: Cc1c(NC2CCC(N)CC2)ccc(N2CCC(C(F)(F)F)CC2)c1F]

The title compound 57 (48.7 mg) was prepared in a total yield of 52.8% as a purple solid with 1:2 mixture by ¹H NMR from tert-butyl (4-((3-fluoro-2-methyl-4-(4-(trifluoromethyl)piperidin-1-yl)phenyl)amino)cyclohexyl)carbama the (117 mg, 0.247 mmol), TFA (1 mL), and DCM (10 mL) according to the procedure for 24. ¹H NMR (400 MHZ, DMSO-d₆) δ 6.70 (td, J=9.2, 5.6 Hz, 1H), 6.29 (dd, J=8.8, 3.6 Hz, 1H), 4.21 (dd, J=108.4, 7.2 Hz, 1H), 3.21-2.79 (m, 4H), 2.59-2.51 (m, 2H), 2.34 (d, J=11.2 Hz, 1H), 2.01 (d, J=2.0 Hz, 1H), 1.97-1.95 (m, 1H), 1.92 (d, J=2.0 Hz, 2H), 1.82 (d, J=14.4 Hz, 3H), 1.71 (dq, J=12.8, 8.8, 8.0 Hz, 2H), 1.55 (ddt, J=15.2, 12.4, 6.4 Hz, 3H), 1.49-1.41 (m, 1H), 1.30-1.16 (m, 2H). Mass (m/z): 3 74.3 [M+H]⁺.

### Compound 58

### N1-(5-fluoro-2-methyl-4-(4-(trifluoromethyl)piperidin-1-yl)phenyl)cyclohexane-1,4-diamine

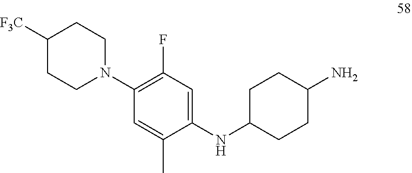 [SMILES: Cc1cc(N2CCC(C(F)(F)F)CC2)c(F)cc1NC1CCC(N)CC1]

The title compound 58 (44.1 mg) was prepared in a total yield of 60.7% as a brown solid with 1:2 mixture by ¹H NMR from tert-butyl (4-((5-fluoro-2-methyl-4-(4-(trifluoromethyl)piperidin-1-yl)phenyl)amino)cyclohexyl)carbama the (92 mg, 0.194 mmol), TFA (1 mL), and DCM (10 mL) according to the procedure for 24. H NMR (400 MHz, DMSO-d₆) δ 6.70 (dd, J=14.8, 9.6 Hz, 1H), 6.34 (dd, J=15.2, 6.4 Hz, 1H), 4.15 (dd, J=104.4, 7.4 Hz, 1H), 3.48-3.30 (m, 2H), 3.20-3.03 (m, 3H), 2.87 (tt, J=11.6, 3.6 Hz, 1H), 2.57 (tt, J=12.0, 3.2 Hz, 2H), 2.37-2.26 (m, 1H), 2.06 (s, 1H), 1.96 (d, J=8.0 Hz, 4H), 1.85-1.68 (m, 4H), 1.61-1.42 (m, 4H), 1.32-1.15 (m, 2H). Mass (m/z): 374.3 [M+H]⁺.

### Compound 59

### N1-(2,5-dimethyl-4-(4-(trifluoromethyl)piperidin-1-yl)phenyl)cyclohexane-1,4-diamine

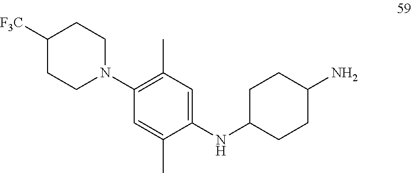 [SMILES: Cc1cc(N2CCC(C(F)(F)F)CC2)c(C)cc1NC1CCC(N)CC1]

The title compound 59 (57.4 mg) was prepared in a total yield of 53.2% as a yellow solid with 1:2 mixture ¹H NMR by from tert-butyl (4-((2,5-dimethyl-4-(4-(trifluoromethyl)piperidin-1-yl)phenyl)amino)cyclohexyl)carbamate (137 mg, 0.292 mmol), TFA (1 mL), and DCM (10 mL) according to the procedure for 24. ¹H NMR (400 MHZ, DMSO-d₆) δ 6.67 (d, J=11.6 Hz, 1H), 6.36 (s, 1H), 3.87 (d, J=75.6 Hz, 1H), 3.07 (d, J=17.6 Hz, 1H), 2.89 (d, J=10.4 Hz, 3H), 2.58-2.50 (m, 2H), 2.34-2.26 (m, 1H), 2.10 (d, J=3.2 Hz, 4H), 2.06 (s, 2H), 1.96 (d, J=7.2 Hz, 4H), 1.84-1.77 (m, 3H), 1.71 (dd, J=7.6, 4.0 Hz, 2H), 1.52 (qd, J=12.5, 10.4, 3.6 Hz, 4H), 1.23 (d, J=12.0 Hz, 1H). Mass (m/z): 370.3 [M+H]⁺.

### Compound 60

### N1-(4-(4-methylpiperidin-1-yl)phenyl)cyclohexane-1,4-diamine

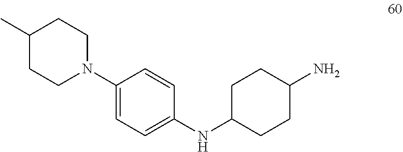 [SMILES: CC1CCN(c2ccc(NC3CCC(N)CC3)cc2)CC1]

The title compound 60 (51.9 mg) was prepared in a yield of 53.7% as a brown solid with 1:1 mixture by ¹H NMR from 4-(4-methylpiperidin-1-yl)aniline (63.9 mg, 0.31 mmol), tert-butyl (4-oxocyclohexyl)carbamate (100 mg, 0.47 mmol) and according to the procedure for 20. ¹H NMR (400 MHZ, DMSO-d₆) δ 8.29 (d, J=33.4 Hz, 3H), 7.82-7.23 (m, 1H), 6.64 (s, 2H), 3.38 (s, 3H), 3.16 (d, J=4.7 Hz, 1H), 3.08 (s, 1H), 2.94 (s, 1H), 2.00 (s, 2H), 1.75 (s, 7H), 1.47 (d, J=12.3 Hz, 2H), 1.23 (s, 1H), 0.95 (d, J=4.6 Hz, 3H). Mass (m/z): 288.5 [M+H]⁺.

### Compound 61

### N1-(4-(4-ethylpiperidin-1-yl)phenyl)cyclohexane-1,4-diamine

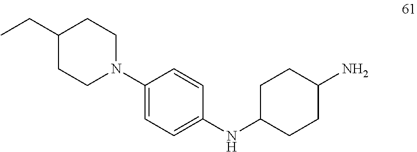 [SMILES: CCC1CCN(c2ccc(NC3CCC(N)CC3)cc2)CC1]

The title compound 61 (38.5 mg) was prepared in a yield of 40.8% as a brown solid with 1:1 mixture by ¹H NMR from 4-(4-ethylpiperidin-1-yl)aniline (63.9 mg, 0.31 mmol), tert-butyl (4-oxocyclohexyl)carbamate (100 mg, 0.47 mmol) and according to the procedure for 20. ¹H NMR (400 MHZ, DMSO-d₆) δ 8.19 (d, J=31.7 Hz, 3H), 7.35 (d, J=94.4 Hz, 1H), 6.56 (s, 3H), 3.33 (s, 4H), 3.12 (d, J=5.0 Hz, 1H), 3.04 (s, 1H), 2.90 (s, 1H), 1.95 (s, 2H), 1.71 (s, 5H), 1.55 (s, 1H), 1.49-1.35 (m, 2H), 1.31-1.02 (m, 4H), 0.85 (t, J=7.2 Hz, 3H). MS (m/z): 302.5 [M+H]⁺.

### Compound 62

### N1-(4-(4,4-dimethylpiperidin-1-yl)phenyl)cyclohexane-1,4-diamine

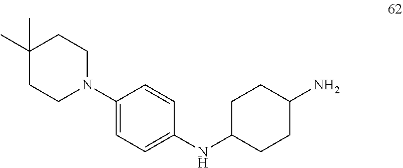 [SMILES: CC1(C)CCN(c2ccc(NC3CCC(N)CC3)cc2)CC1]

The title compound 62 (31.5 mg) was prepared in a yield of 35.8% as a brown solid with 1:1 mixture by ¹H NMR from 4-(4,4-dimethylpiperidin-1-yl)aniline (59 mg, 0.31 mmol), tert-butyl (4-oxocyclohexyl)carbamate (100 mg, 0.47 mmol) and according to the procedure for 20. ¹H NMR (400 MHZ, DMSO-d₆) δ 8.20 (d, J=30.2 Hz, 3H), 6.78 (s, 1H), 6.56 (s, 2H), 3.17 (d, J=5.1 Hz, 1H), 2.95 (s, 4H), 1.99 (s, 2H), 1.74 (s, 3H), 1.65-1.33 (m, 6H), 1.24 (s, 2H), 0.96 (s, 6H). Mass (m/z): 302.6 [M+H]⁺.

### Compound 63

### N1-(4-(4-isopropylpiperidin-1-yl)phenyl)cyclohexane-1,4-diamine

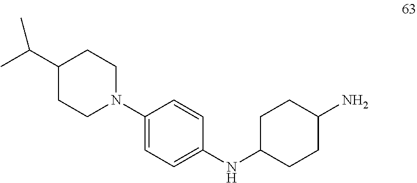 [SMILES: CC(C)C1CCN(c2ccc(NC3CCC(N)CC3)cc2)CC1]

The title compound 63 (40.5 mg) was prepared in a yield of 40.4% as a brown solid with 1:1 mixture by ¹H NMR from 4-(4-isopropylpiperidin-1-yl)aniline (68 mg, 0.31 mmol), tert-butyl (4-oxocyclohexyl)carbamate (100 mg, 0.47 mmol) and according to the procedure for 20. ¹H NMR (400 MHZ, DMSO-d₆) δ 8.17-7.95 (m, 3H), 7.28 (s, 2H), 6.68 (s, 2H), 3.14 (s, 1H), 2.99 (s, 1H), 2.23-2.13 (m, 1H), 1.98 (s, 2H), 1.78 (dd, J=27.0, 10.1 Hz, 7H), 1.62 (s, 1H), 1.46 (s, 3H), 1.23 (s, 2H), 0.89 (d, J=6.5 Hz, 6H). Mass (m/z): 316.6 [M+H]⁺.

### Compound 64

### tert-butyl (4-((2R,6S)-2,6-dimethyltetrahydro-2H-pyran-4-yl)phenyl)carbamate

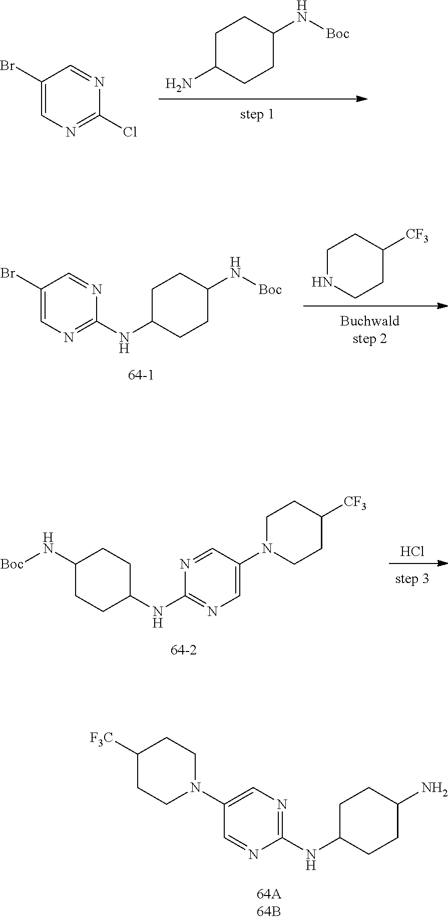

Step 1. Preparation of tert-butyl (4-((5-bromopyrimidin-2-yl)amino)cyclohexyl)carbamate (64-1)

To a solution of 5-bromo-2-chloropyrimidine (0.5 g, 2.62 mmol) in DMF (10 mL) was added tert-butyl (4-aminocyclohexyl)carbamate (0.67 g, 3.14 mmol) and Cs₂CO₃ (2.56 g, 7.86 mmol) the mixture was stirred at 90° C. overnight. Diluting with water (30 mL), the reaction was extracted by EA (20 mL) for 3 times. The organic phase was washed 3 times by NaCl aq (20 mL), dried over sodium sulfate, concentrated under vacuum and the residue was purified by flash chromatography to afford the desired product (0.4 g, 41.2%) as yellow oil. Mass (m/z): 371.2 [M+H]⁺.

Step 2. Preparation of tert-butyl (4-((5-(4-(trifluoromethyl)piperidin-1-yl)pyrimidin-2-yl)amino)cyclohexyl)carbamate (64-2)

To a solution of tert-butyl (4-((5-bromopyrimidin-2-yl)amino)cyclohexyl)carbamate (0.2 g, 0.5 mmol) in dioxane (5 mL) was added 4-(trifluoromethyl)piperidine (320 mg, 2 mmol), Cs₂CO₃ (0.5 g, 1.53 mmol), Ruphos (70 mg, 0.1 mmol) and Pd2 (dba) 3 (265 mg, 0.23 mmol). Then the mixture was stirred at 100° C. for 12 h. Quenched with water (10 mL), the reaction was extracted by EA (10 mL) for 3 times, dried over sodium sulfate, concentrated under vacuum and the residue was purified by flash chromatography to afford the desired product (0.16 g, 50.2%) as colorless oil. Mass (m/z): 248.2 [M+H]⁺.

Step 3. Preparation of tert-butyl (4-((2R,6S)-2,6-dimethyltetrahydro-2H-pyran-4-yl)phenyl)carbamate (64)

To a solution of tert-butyl (4-((2R,6S)-2,6-dimethyl-3,6-dihydro-2H-pyran-4-yl)phenyl)carbamate (0.16 g, 0.36 mmol) in THF (5 ml) was added HCl in dioxane (5 mL) and the mixture was stirred for 2 h. Quenched with NaHCO₃ (10 mL), the reaction was extracted by EA (10 mL) for 3 times, dried over sodium sulfate, concentrated under vacuum and the residue was purified by prep-HPLC (column-Xbridge-C18 150×21.2 mm, 5 μm; Mobile phase: ACN-H₂O (0.1% FA), 40%-60%) to afford the desired product 64A (14.5 mg) as a white solid and 64B (20.2 mg) as a white solid. 64A: ¹H NMR (400 MHZ, CD₃OD) δ 8.54 (s, 1H), 8.10 (s, 2H), 3.70-3.66 (m, 1H), 3.44 (d, J=12.0 Hz, 2H), 3.18-3.03 (m, 1H), 2.68 (m, 2H), 2.34-2.18 (m, 1H), 2.19-2.02 (m, 4H), 1.97 (d, J=12.8 Hz, 2H), 1.71 (m, 2H), 1.52 (m, 2H), 1.44-1.29 (m, 2H). Mass (m/z): 344.2 [M+H]⁺. HPLC: Rt: 4.333 min (Column: XBRIDGE 2.1*50 mm, 3.5 μm; Mobile Phase: H₂O (0.05% TFA) ACN (0.05% TFA), ACN from 0% to 60% over 7 minutes, 7-8 min, ACN from 60% to 100%; 0.8 mL/min). 64B: ¹H NMR (400 MHZ, CD₃OD) δ=8.53 (s, 1H), 8.13 (s, 2H), 3.95 (s, 1H), 3.46 (d, J=12.0 Hz, 2H), 3.27-3.19 (m, 1H), 2.74-2.63 (m, 2H), 2.34-2.21 (m, 1H), 1.99-1.86 (m, 6H), 1.81-1.66 (m, 6H). Mass (m/z): 344.2 [M+H]⁺. HPLC: Rt: 4.488 min (Column: XBRIDGE 2.1*50 mm, 3.5 μm; Mobile Phase: H₂O (0.05% TFA) ACN (0.05% TFA), ACN from 0% to 60% over 7 minutes, 7-8 min, ACN from 60% to 100%; 0.8 mL/min).

### Compound 65

### N1-(4-methyl-2-(4-(trifluoromethyl)piperidin-1-yl)pyrimidin-5-yl)cyclohexane-1,4-diamine

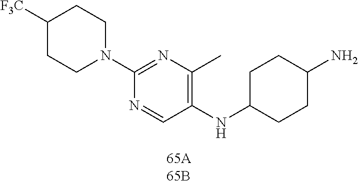 [SMILES: Cc1nc(N2CCC(C(F)(F)F)CC2)ncc1NC1CCC(N)CC1]

The title compounds 65A (6.2 mg, 13%) as a white solid and 65B (11.5 mg, 28%) as a white solid were prepared from To a solution of tert-butyl (4-((4-methyl-2-(4-(trifluoromethyl)piperidin-1-yl)pyrimidin-5-yl)amino)cyclohexyl)carbamat e (53 mg, 0.12 mmol), HCl in dioxane (4N; 3 mL) and CH₂Cl₂ (3 mL) according to the procedure for 64. 65A: ¹H NMR (400 MHZ, CD₃OD) δ 7.77 (s, 1H), 4.67 (d, J=13.3 Hz, 2H), 2.94-2.88 (m, 1H), 2.80 (td, J=13.1, 2.4 Hz, 2H), 2.51-2.34 (m, 1H), 2.34 (s, 3H), 1.87 (d, J=1.4 Hz, 2H), 1.79-1.68 (m, 6H), 1.65-1.56 (m, 2H), 1.53-1.46 (m, 3H). Mass (m/z): 358.2 [M+H]⁺. HPLC: Rt: 3.792 min (Column: XBRIDGE 2.1*50 mm, 3.5 μm; Mobile Phase: H₂O (0.05% TFA)/ACN (0.05% TFA), ACN from 0% to 60% over 7 minutes, 7-8 min, ACN from 60% to 100%; 0.8 mL/min). 65B: ¹H NMR (400 MHZ, CD₃OD) δ 7.78 (s, 1H), 4.73-4.61 (m, 2H), 3.12-3.00 (m, 1H), 2.86-2.77 (m, 2H), 2.76-2.67 (m, 1H), 2.48-2.34 (m, 1H), 2.29 (s, 3H), 2.12-1.99 (m, 2H), 1.99-1.83 (m, 4H), 1.53-1.46 (m, 2H), 1.36-1.25 (m, 4H). Mass (m/z): 358.2 [M+H]⁺. HPLC: Rt: 3.855 min (Column: XBRIDGE 2.1*50 mm, 3.5 μm; Mobile Phase: H₂O (0.05% TFA)/ACN (0.05% TFA), ACN from 0% to 60% over 7 minutes, 7-8 min, ACN from 60% to 100%; 0.8 mL/min).

### Compound 66

### N1-(3-methyl-4-(4-(trifluoromethyl)piperidin-1-yl)phenyl)cyclohexane-1,4-diamine

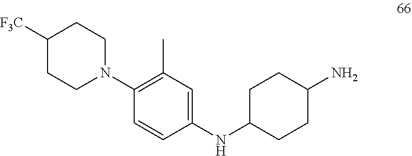 [SMILES: Cc1cc(NC2CCC(N)CC2)ccc1N1CCC(C(F)(F)F)CC1]

The title compound 66 (53.1 mg) was prepared in a total yield of 53.2% as a purple solid with 1:2 mixture by ¹H NMR from tert-butyl (4-((3-methyl-4-(4-(trifluoromethyl)piperidin-1-yl)phenyl)amino)cyclohexyl)carbamate (128 mg, 0.281 mmol), TFA (1 mL), and DCM (10 mL) according to the procedure for 24. ¹H NMR (400 MHZ, DMSO-d₆) δ 6.77 (t, J=7.6 Hz, 1H), 6.54-6.28 (m, 2H), 3.08-2.97 (m, 1H), 2.93-2.86 (m, 2H), 2.56-2.48 (m, 2H), 2.34-2.25 (m, 1H), 2.08 (dd, J=8.4, 2.3 Hz, 3H), 1.96 (dt, J=13.2, 7.2 Hz, 3H), 1.85-1.65 (m, 6H), 1.61-1.35 (m, 5H). Mass (m/z): 356.3 [M+H]⁺.

### Compound 67

### N1-(3-chloro-4-(4-(trifluoromethyl)piperidin-1-yl)phenyl)cyclohexane-1,4-diamine

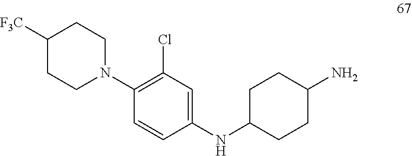 [SMILES: NC1CCC(Nc2ccc(N3CCC(C(F)(F)F)CC3)c(Cl)c2)CC1]

The title compound 67 (33.1 mg) was prepared in a total yield of 52.4% as a purple solid with 1:2 mixture by ¹H NMR from tert-butyl (4-((3-chloro-4-(4-(trifluoromethyl)piperidin-1-yl)phenyl)amino)cyclohexyl)carbamate (80 mg, 0.168 mmol), TFA (1 mL), and DCM (10 mL) according to the procedure for 24. ¹H NMR (400 MHZ, DMSO-d₆) δ 6.88 (dd, J=8.8, 6.4 Hz, 1H), 6.62 (dd, J=21.2, 2.4 Hz, 1H), 6.48 (ddd, J=14.8, 8.7, 2.7 Hz, 1H), 5.50 (d, J=6.4 Hz, 1H), 3.32 (t, J=4.4 Hz, 1H), 3.12-2.98 (m, 3H), 2.96-2.81 (m, 1H), 2.53 (tt, J=11.6, 2.4 Hz, 2H), 2.38-2.27 (m, 1H), 1.92 (dt, J=17.2, 13.6 Hz, 3H), 1.84-1.65 (m, 5H), 1.59-1.40 (m, 4H). Mass (m/z): 376.3 [M+H]⁺.

### Compound 68

### N1-(3-fluoro-4-(4-(trifluoromethyl)piperidin-1-yl)phenyl)cyclohexane-1,4-diamine

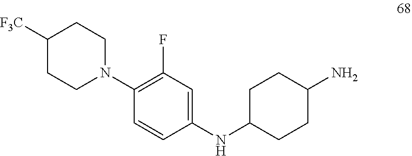 [SMILES: NC1CCC(Nc2ccc(N3CCC(C(F)(F)F)CC3)c(F)c2)CC1]

The title compound 68 (84.4 mg) was prepared in a total yield of 78.8% as a purple solid with 1:2 mixture by ¹H NMR from tert-butyl (4-((3-fluoro-4-(4-(trifluoromethyl)piperidin-1-yl)phenyl)amino)cyclohexyl)carbamate (137 mg, 0.299 mmol), TFA (1 mL), and DCM (10 mL) according to the procedure for 24. ¹H NMR (400 MHZ, DMSO-d₆) δ 6.85-6.72 (m, 1H), 6.44-6.23 (m, 2H), 5.46 (s, 1H), 3.35-3.27 (m, 1H), 3.12 (d, J=11.4 Hz, 2H), 3.02 (s, 1H), 2.90 (s, 1H), 2.61-2.51 (m, 2H), 2.39-2.26 (m, 1H), 2.02-1.89 (m, 3H), 1.85-1.65 (m, 5H), 1.63-1.50 (m, 3H), 1.50-1.33 (m, 2H). Mass (m/z): 360.3 [M+H]⁺.

### Compound 69

### N1-(2-methyl-4-(4-(trifluoromethyl)piperidin-1-yl)phenyl)cyclohexane-1,4-diamine

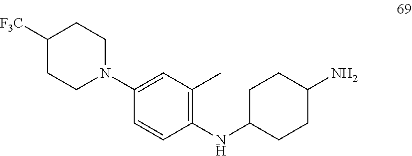 [SMILES: Cc1cc(N2CCC(C(F)(F)F)CC2)ccc1NC1CCC(N)CC1]

The title compound 69 (84.4 mg) was prepared in a total yield of 81.9% as a purple solid with 1:2 mixture 1H by NMR from tert-butyl (4-((2-methyl-4-(4-(trifluoromethyl)piperidin-1-yl)phenyl)amino)cyclohexyl)carbamate (132 mg, 0.290 mmol), TFA (1 mL), and DCM (10 mL) according to the procedure for 24. ¹H NMR (400 MHZ, DMSO-d₆) δ 6.83-6.56 (m, 2H), 6.46 (s, 1H), 3.99-3.67 (m, 1H), 3.45 (d, J=29.6 Hz, 3H), 3.05 (s, 1H), 2.89 (s, 1H), 2.34 (s, 1H), 2.15-1.89 (m, 6H), 1.88-1.65 (m, 6H), 1.63-1.35 (m, 5H). Mass (m/z): 356.3 [M+H]⁺.

### Compound 70

### N1-(2-chloro-4-(4-(trifluoromethyl)piperidin-1-yl)phenyl)cyclohexane-1,4-diamine

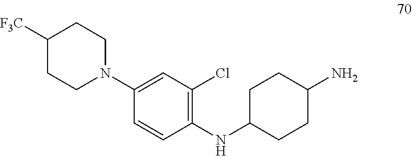 [SMILES: NC1CCC(Nc2ccc(N3CCC(C(F)(F)F)CC3)cc2Cl)CC1]

The title compound 70 (101.0 mg) was prepared in a total yield of 83.5% as a purple solid with 1:2 mixture by ¹H NMR from tert-butyl (4-((3-chloro-4-(4-(trifluoromethyl)piperidin-1-yl)phenyl)amino)cyclohexyl)carbamate (153 mg, 0.322 mmol), TFA (1 mL), and DCM (10 mL) according to the procedure for 24. ¹H NMR (400 MHZ, DMSO-d₆) δ 6.88 (dd, J=13.2, 2.7 Hz, 1H), 6.79 (ddd, J=9.6, 6.8, 2.8 Hz, 1H), 6.68 (dd, J=9.2, 4.8 Hz, 1H), 4.17 (dd, J=40.4, 8.0 Hz, 1H), 3.49-3.43 (m, 2H), 3.18-3.05 (m, 1H), 2.88 (s, 1H), 2.53 (ddd, J=12.8, 3.6, 2.0 Hz, 2H), 2.39-2.30 (m, 1H), 2.04-1.88 (m, 3H), 1.85-1.60 (m, 6H), 1.59-1.38 (m, 4H). Mass (m/z): 376.3 [M+H]⁺.

### Compound 71

### N1-(2-fluoro-4-(4-(trifluoromethyl)piperidin-1-yl)phenyl)cyclohexane-1,4-diamine

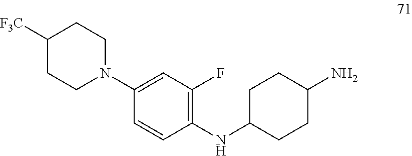 [SMILES: NC1CCC(Nc2ccc(N3CCC(C(F)(F)F)CC3)cc2F)CC1]

The title compound 71 (64.2 mg) was prepared in a total yield of 76.2% as a purple solid with 1:2 mixture by ¹H NMR from tert-butyl (4-((3-fluoro-4-(4-(trifluoromethyl)piperidin-1-yl)phenyl)amino)cyclohexyl)carbamate (107 mg, 0.233 mmol), TFA (1 mL), and DCM (10 mL) according to the procedure for 24. ¹H NMR (400 MHz, DMSO-d₆) δ 6.67 (td, J=28.8, 16.4 Hz, 3H), 4.57-4.11 (m, 1H), 3.48 (t, J=9.6 Hz, 2H), 3.07 (s, 1H), 2.87 (s, 1H), 2.59-2.48 (m, 2H), 2.37 (dt, J=22.4, 7.6 Hz, 1H), 2.02-1.86 (m, 3H), 1.85-1.65 (m, 4H), 1.60-1.36 (m, 4H), 1.20 (q, J=12.4 Hz, 2H). Mass (m/z): 360.3 [M+H]⁺.

### Compound 72

### N-(4-cyclohexylphenyl)-1-ethylpiperidin-4-amine

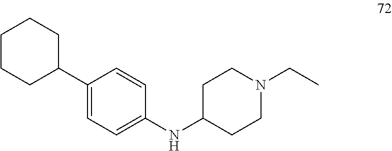 [SMILES: CCN1CCC(Nc2ccc(C3CCCCC3)cc2)CC1]

The title compound 72 (115 mg, 70.5%) was obtained as brown oil from 4-cyclohexylaniline (100 mg, 0.57 mmol), DCE (5 mL), 1-ethylpiperidin-4-one (87 mg, 0.69 mmol), acetic acid (3.42 mg, 0.057 mmol), and sodium triacetoxyborohydride (363 mg, 1.71 mmol) according to the procedure for 5. ¹H NMR (300 MHz, DMSO-d₆) δ=6.87 (d, J=8.4 Hz, 2H), 6.46 (d, J=8.4 Hz, 2H), 5.15 (s, 1H), 2.80 (d, J=11.4 Hz, 2H), 2.52-2.47 (m, 2H), 2.33-2.23 (m, 2H), 1.97 (t, J=11.4 Hz, 2H), 1.83 (s, 1H), 1.70 (d, J=11.4 Hz, 5H), 1.27 (m, 7H), 0.97 (t, J=7.2 Hz, 3H). Mass (m/z): 287.3 [M+H]⁺.

### Compound 73

### N-(4-(4,4-dimethylpiperidin-1-yl)phenyl)-1-methylpiperidin-4-amine

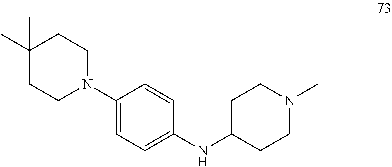

The title compound 73 (6.1 mg) was prepared in a total yield of 8.1% as a pink solid with 1:2 mixture by ¹H NMR from 4-(4,4-dimethylcyclohexyl)aniline (50 mg, 0.246 mmol), 1-methylpiperidin-4-one (33 mg, 0.295 mmol), AcOH (0.1 mL), NaBH(OAc)₃ (104 mg, 0.493 mmol) and DCE (10 mL) according to the procedure for 5. ¹H NMR (400 MHZ, Chloroform-d) δ 6.96-6.90 (m, 2H), 6.54-6.47 (m, 2H), 5.44-5.27 (m, 1H), 3.12 (d, J=11.6 Hz, 3H), 2.68 (s, 2H), 2.22 (tt, J=10.0, 5.2 Hz, 1H), 1.98 (dt, J=14.4, 4.0 Hz, 2H), 1.64-1.37 (m, 9H), 1.31-1.21 (m, 3H), 0.93 (d, J=8.4 Hz, 6H). Mass (m/z): 301.3 [M+H]⁺.

### Compound 74

### N1-(4-(4,4-dimethylcyclohexyl)phenyl)cyclohexane-1,4-diamine

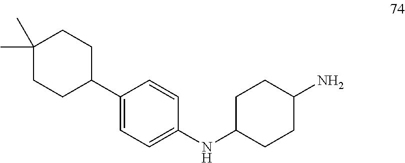 [SMILES: CC1(C)CCC(c2ccc(NC3CCC(N)CC3)cc2)CC1]

The title compound 74 (130 mg, 62.8%) as a white solid was prepared from tert-butyl (4-((4-(4,4-dimethylcyclohexyl)phenyl)amino)cyclohexyl)carbamate (180 mg, 0.45 mmol) and HCl in 1,4-dioxane (10 mL, 4 N) according to the procedure for 37. ¹H NMR (400 MHZ, DMSO-d₆) δ 8.10 (brs, 2H), 7.36 (brs, 3H), 3.80-3.57 (m, 3H), 3.49-3.25 (m, 1H), 2.96 (br, 1H), 2.44-2.37 (m, 1H), 2.04-1.63 (m, 4H), 1.61-1.46 (m, 4H), 1.31 (m, 6H), 0.91 (d, J=12.0 Hz, 6H). MS (m/z) 301.3.

### Compound 75

### N-(4-cyclohexylphenyl)-1-ethylpiperidin-4-amine

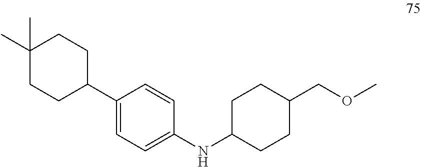

The title compound 75 (23 mg, 14.2%) was prepared as yellow oil from 4-(methoxymethyl)cyclohexan-1-one (84 mg, 0.59 mmol), DCE (5 mL), 4-(4,4-dimethylcyclohexyl)aniline (100 mg, 0.49 mmol) and acetic acid (2.9 mg, 0.059 mmol) according to the procedure for 5. ¹H NMR (300 MHz, CDCl₃) δ 7.07-6.98 (m, 2H), 6.59-6.50 (m, 2H), 3.35 (d, J=2.0 Hz, 3H), 3.24 (dd, J=12.3 Hz, 9.9 Hz, 2H), 2.33-2.25 (m, 1H), 1.87-1.84 (d, J=6.9 Hz, 1H), 1.74-1.54 (m, 8H), 1.47 (d, J=14.4 Hz, 2H), 1.38-1.24 (m, 4H), 1.09 (d, J=9.9 Hz, 2H), 0.96-0.94 (m, 6H). Mass (m/z): 330.3 [M+H]⁺.

### Compound 76

### (4-(dimethylamino)cyclohexyl)-4-((4-(4,4-dimethylcyclohexyl)phenyl)amino)cyclohexane-1-carboxamide

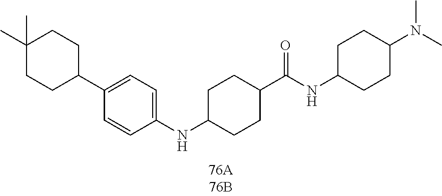

The desired products 76A (Rt=4.89 min; 4.2 mg, 20%) as a white solid and 76B (Rt=4.78 min; 4.0 mg, 19%) as a white solid were prepared from 4-((4-(4,4-dimethylcyclohexyl)phenyl)amino)cyclohexane-1-carboxylic acid (15 mg, 0.046 mmol), N1,N1-dimethylcyclohexane-1,4-diamine (7.1 mg, 0.05 mmol), DIEA (16.5 mg, 0.05 mmol) in DMF (1 mL) and DMT-MM (14.8 mg, 0.05 mmol) according to the procedure for 1, which were purified by preparative HPLC (Column: X Select-CSH-Prep 5 μm OBD, 19*150 mm; ACN/water (5% % TFA)=10%-30%-95%-95%-10%, 0-7 min-7.5 min-8.5 min-10.0 min). 76A: ¹H NMR (400 MHZ, DMSO-d₆) δ 8.06 (d, J=7.6 Hz, 1H), 7.77 (d, J=7.6 Hz, 1H), 6.93 (m, 2H), 6.49 (m, 2H), 3.93-3.76 (m, 1H), 3.63-3.54 (m, 1H), 3.18-3.04 (m, 2H), 2.68 (d, J=4.8 Hz, 6H), 2.23 (m, 1H), 2.03-1.63 (m, 10H), 1.58-1.36 (m, 8H), 1.35-1.18 (m, 4H), 1.08 (m, 2H), 0.93 (d, J=8.4 Hz, 6H). Mass (m/z): 454.3 [M+H]⁺. 76B: ¹H NMR (400 MHZ, DMSO-d₆) δ 8.05 (d, J=7.6 Hz, 1H), 7.70 (d, J=7.6 Hz, 1H), 6.93 (m, 2H), 6.48 (m, 2H), 3.82 (m, 1H), 3.71 (s, 1H), 3.55-3.42 (m, 1H), 3.13 (m, 1H), 2.67 (d, J=4.8 Hz, 6H), 2.22 (m, 1H), 2.05-1.65 (m, 10H), 1.91-1.63 (m, 8H), 1.59-1.01 (m, 6H), 0.93 (d, J=8.4 Hz, 6H). Mass (m/z): 454.3 [M+H]⁺.

### Compound 77

### 4-((4-(4,4-dimethylcyclohexyl)phenyl)amino)-N-(1-methylpiperidin-4-yl)cyclohexane-1-carbox amide

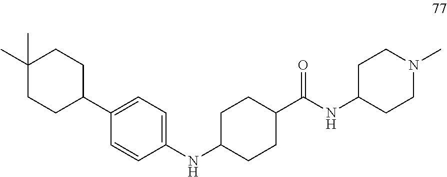

The title compound 77 (7.2 mg) was prepared in a total yield of 37% as a yellow solid with 1:2 mixture ¹H NMR by from 4-((4-(4,4-dimethylcyclohexyl)phenyl)amino)cyclohexane-1-carboxylic acid (15 mg, 0.046 mmol), 1-methylpiperidin-4-amine (5.7 mg, 0.05 mmol), DIEA (16.5 mg, 0.05 mmol) and DMT-MM (14.8 mg, 0.05 mmol) according to the procedure for 1. ¹H NMR (400 MHZ, DMSO-d₆) δ 8.11 (d, J=8.0 Hz, 1H), 7.96 (d, J=8.0 Hz, 1H), 6.93 (m, 2H), 6.51 (m, 2H), 3.93 (m, 1H), 3.82 (m, 1H), 3.72 (m, 1H), 3.65-3.55 (m, 1H), 3.31-2.91 (m, 3H), 2.69 (s, 1H), 2.69 (s, 2H), 2.28-2.13 (m, 1H), 2.13-1.63 (m, 8H), 1.62-1.37 (m, 6H), 1.36-1.17 (m, 4H), 1.14-1.05 (m, 2H), 0.94 (s, 3H), 0.92 (s, 3H). Mass (m/z): 426.2 [M+H]⁺.

### Compound 78

### N-(4-(aminomethyl)cyclohexyl)-4-(4,4-dimethylcyclohexyl)aniline

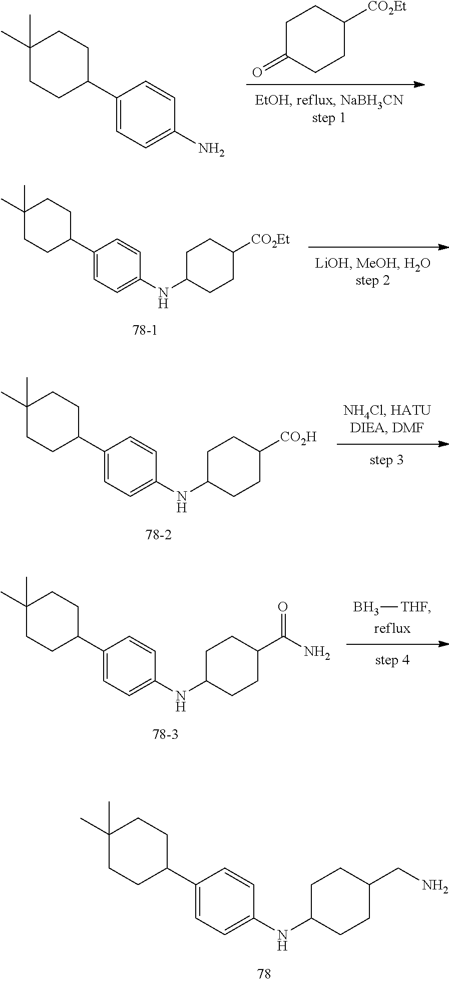

Step 1. Preparation of ethyl 4-((4-(4,4-dimethylcyclohexyl)phenyl)amino)cyclohex ane-1-carboxylate (78-1)

A mixture of 4-(4,4-dimethylcyclohexyl)aniline (0.4 g, 1.97 mmol) and ethyl 4-oxocyclohexane-1-carboxylate (0.335 g, 1.97 mmol) in EtOH was stirred at 90° C. for 1 h. Then the mixture was added NaBH₃CN (0.37 g, 5.91 mmol). The mixture was stirred at 25° C. for 4 h. The mixture was quenched with H₂O and extracted with EA. The organic phase was concentrated and purified by a silica gel column chromatography, eluted with PE/EA=2:1 to give 78-1 as yellow oil (0.36 g, 51%). Mass (m/z): 358.3 [M+H]⁺.

Step 2. Preparation of 4-((4-(4,4-dimethylcyclohexyl)phenyl)amino)cyclohexane-1-carboxylic acid (78-2)

To a solution of 78-1 (0.36 g, 1 mmol) in MeOH (10 mL) was added LiOH H₂O (0.13 g, 3 mmol) at 25° C. The reaction was stirred at 25° C. for 6 hrs. The pH of the mixture was adjusted to 3 with 1 N HCl, extracted with EA. The organic phase was concentrated to give 78-2 as yellow oil (0.28 g, 85.1%). Mass (m/z): 329.9 [M+H]⁺.

Step 3. Preparation of 4-((4-(4,4-dimethylcyclohexyl)phenyl)amino)cyclohexane-1-carboxamide (78-3)

To a solution of 78-2 (0.18 g, 0.55 mmol) and HATU (0.21 g, 0.55 mmol) in DMF (5 mL) was added NH₄Cl (87 mg, 1.65 mmol) and DIEA (0.21 g, 1.65 mmol) at 25° C. The reaction was stirred at 25° C. for 16 hrs. The mixture was diluted with EA and washed with water. The organic phase was concentrated and purified by a silica gel column chromatography, eluted with PE/EA=1:1 to give 78-3 as a yellow solid (0.11 g, 61%). Mass (m/z): 328.9 [M+H]⁺.

Step 4. Preparation of N-(4-(aminomethyl)cyclohexyl)-4-(4,4-dimethylcyclohex yl)aniline (78) To a solution of 78-3 (0.15 g, 0.46 mmol) in THF (1 mL) was added BH₃-THF (10 mL). The reaction was stirred at 70° C. for 16 h. The mixture was quenched by MeOH and water, concentrated and extracted with EA. The organic phase was concentrated and purified by prep-TLC, eluted with DCM: MeOH=9:1 (0.5% NH₃. H₂O) to give 78 (82 mg, 57.3%) as a yellow solid. ¹H NMR (300 MHz, CD₃OD) δ 6.99 (d, J=8.4 Hz, 2H), 6.69-6.53 (m, 2H), 3.56 (d, J=5.7 Hz, 2H), 2.90-2.87 (m, 1H), 2.29-2.11 (m, 1H), 1.77-1.13 (m, 17H), 0.95 (d, J=12 Hz, 6H). Mass (m/z): 315.0 [M+H]⁺.

### Compound 79

### 4-(4,4-dimethylcyclohexyl)-N-(2-(2-methoxyethoxy)ethyl)aniline

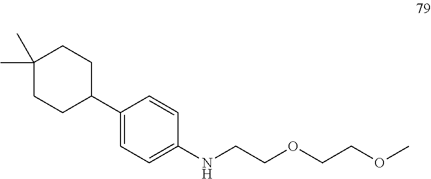

The title compound 79 (34.4 mg) was prepared in a yield of 41.9% as a pale yellow powder from 4-(4,4-dimethylcyclohexyl)aniline (50 mg, 0.25 mmol), 1-iodo-2-(2-methoxyethoxy) ethane (56 mg, 0.25 mmol) and potassium carbonate (41 mg, 0.3 mmol) according to the procedure for 12. ¹H NMR (400 MHZ, DMSO-d₆) δ 7.19 (s, 2H), 7.00 (s, 2H), 3.65-3.39 (m, 5H), 3.24 (s, 3H), 2.35 (s, 1H), 1.98 (dt, J=13.0, 7.0 Hz, 1H), 1.60-1.50 (m, 4H), 1.43 (d, J=12.9 Hz, 2H), 1.30 (t, J=8.8 Hz, 2H), 1.23 (s, 2H), 0.95 (s, 3H), 0.92 (s, 3H). Mass (m/z): 306.4 [M+H]⁺.

### Compound 80

### N-(4-(aminomethyl)cyclohexyl)-6-(4,4-dimethylcyclohexyl)-2-methylpyridin-3-amine

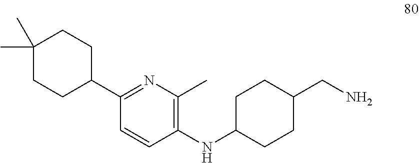 [SMILES: Cc1nc(C2CCC(C)(C)CC2)ccc1NC1CCC(CN)CC1]

The title compound 80 (3.0 mg) was prepared in a total yield of 3.0% as a yellow solid with 1:1 mixture by ¹H NMR. from tert-butyl ((4-((6-(4,4-dimethylcyclohexyl)-2-methylpyridin-3-yl)amino)cyclohexyl)methyl)carbamate (16.0 mg, 37.2 μmol) and TFA (3 mL) according to the procedure for 24. ¹H NMR (400 MHZ, DMSO-d₆) δ 7.91 (s, 3H), 7.20 (s, 2H), 2.81-2.76 (m, 1H), 2.52 (s, 3H), 2.46-2.38 (m, 2H), 1.99-1.92 (m, 1H), 1.86-1.73 (m, 2H), 1.67-1.51 (m, 9H), 1.47-1.41 (m, 1H), 1.33-1.25 (m, 3H), 1.13-1.05 (m, 1H), 0.95 (d, J=9.4 Hz, 6H). Mass (m/z): 330.4 [M+H]⁺.

### Compound 81

### N-(4-(aminomethyl)cyclohexyl)-4-(pentan-3-yl)aniline

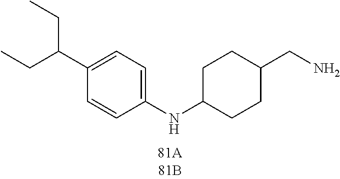 [SMILES: CCC(CC)c1ccc(NC2CCC(CN)CC2)cc1]

The desired product 81A (Rt=9.0 min) as a white solid (8.2 mg, 11.4%) and 81B (Rt=10.4 min) as a white solid (34.1 mg, 48.8%) were prepared from tert-butyl ((4-((4-(pentan-3-yl)phenyl)amino)cyclohexyl)methyl)carbamate(96 mg, 0.277 mmol), DCM (10 mL) and TFA (1 mL) according to the procedure for 24, which were purified by prep HPLC (solvent system (Column: X Select-CSH-Prep 5 μm OBD, 19*150 mm; ACN/water (0.5% TFA)=15%-25%-95%-95%-15%, 0-10 min-10.5 min-11.5 min-13 min). 81A: ¹H NMR (400 MHZ, DMSO-d₆) δ 7.81 (s, 2H), 7.21-6.93 (m, 4H), 2.69-2.58 (m, 2H), 2.25 (s, 1H), 1.90 (d, J=12.4 Hz, 2H), 1.79 (d, J=12.8 Hz, 2H), 1.67-1.36 (m, 5H), 1.26 (s, 2H), 0.98 (q, J=12.4 Hz, 2H), 0.66 (t, J=7.2 Hz, 6H). Mass (m/z): 275.3 [M+H]⁺. 81B: ¹H NMR (400 MHz, DMSO-d₆) δ 7.86 (s, 2H), 7.21-6.93 (m, 4H), 3.44 (td, J=7.2, 3.6 Hz, 1H), 2.75 (p, J=6.0 Hz, 2H), 2.25 (dp, J=9.2, 5.2 Hz, 1H), 1.75 (s, 1H), 1.66-1.36 (m, 12H), 0.66 (t, J=7.2 Hz, 6H). Mass (m/z): 275.3 [M+H]⁺.

### Compound 82

### N-(4-(aminomethyl)cyclohexyl)-4-cyclohexylaniline

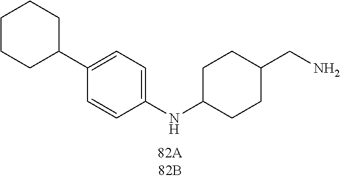 [SMILES: NCC1CCC(Nc2ccc(C3CCCCC3)cc2)CC1]

The desired products 82A (Rt=8.3 min) as yellow oil (13.4 mg, 10.7%) and 82B (Rt=10.7 min) as a white solid (57.6 mg, 46.0%) was prepared from tert-butyl ((4-((4-cyclohexylphenyl)amino)cyclohexyl)methyl)carbamate (169 mg, 0.438 mmol), DCM (10 mL) and TFA (1 mL) according to the procedure for 24, which were purified by prep HPLC (solvent system (ACN/water (0.5% TFA)=15%-25%-95%-95%-15%, 0-13 min-13.5 min-14.5 min-16 min). 82A: ¹H NMR (400 MHZ, DMSO-d₆) δ 7.90-7.67 (m, 3H), 7.16 (s, 2H), 6.99 (s, 2H), 3.19 (s, 1H), 2.63 (p, J=6.0 Hz, 2H), 2.41 (s, 1H), 1.91 (dd, J=13.2, 4.0 Hz, 2H), 1.80-1.69 (m, 6H), 1.69-1.62 (m, 1H), 1.49 (ddh, J=12.0, 7.9, 4.4 Hz, 1H), 1.32 (dd, J=12.0, 9.2 Hz, 4H), 1.27-1.18 (m, 3H), 0.97 (qd, J=13.2, 3.2 Hz, 2H). Mass (m/z): 287.3 [M+H]⁺. 82B: ¹H NMR (400 MHZ, DMSO-d₆) δ 7.81 (s, 3H), 7.14 (s, 2H), 6.96 (s, 2H), 3.42 (dq, J=7.2, 4.0 Hz, 1H), 2.74 (q, J=5.6, 5.2 Hz, 2H), 2.41 (d, J=11.6 Hz, 1H), 1.79-1.44 (m, 15H), 1.31 (t, J=10.4 Hz, 4H). Mass (m/z): 287.3 [M+H]⁺.

### Compound 83

### N-(3-(aminomethyl)cyclopentyl)-4-(4,4-dimethylcyclohexyl)aniline

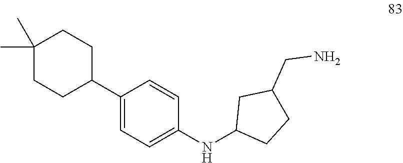 [SMILES: CC1(C)CCC(c2ccc(NC3CCC(CN)C3)cc2)CC1]

The title compound 83 (18.1 mg) was prepared in a yield of 24.5% as a pale yellow powder with 1:1 mixture by ¹H NMR from 4-(4,4-dimethylcyclohexyl)aniline (50 mg, 0.25 mmol), tert-butyl ((3-oxocyclopentyl)methyl)carbamate (79 mg, 0.37 mmol) and NaBH (OAc); (104 mg, 0.49 mmol) according to the procedure for 20. ¹H NMR (400 MHZ, DMSO-d₆) δ 7.91 (s, 2H), 6.95 (d, J=7.9 Hz, 2H), 6.49 (s, 2H), 5.54-5.27 (m, 1H), 3.78-3.64 (m, 1H), 2.77 (dd, J=12.5, 6.3 Hz, 2H), 2.25 (dq, J=12.7, 7.1 Hz, 2H), 2.08-1.96 (m, 1H), 1.89 (ddd, J=14.4, 9.2, 5.6 Hz, 1H), 1.83-1.72 (m, 1H), 1.53 (dd, J=10.4, 3.4 Hz, 4H), 1.50-1.38 (m, 4H), 1.30 (dd, J=12.1, 5.2 Hz, 2H), 1.11 (q, J=8.5 Hz, 1H), 0.95 (s, 3H), 0.93 (s, 3H). Mass (m/z): 301.6 [M+H]⁺.

### Compound 84

### N-(3-(aminomethyl)cyclobutyl)-4-(4,4-dimethylcyclohexyl)aniline

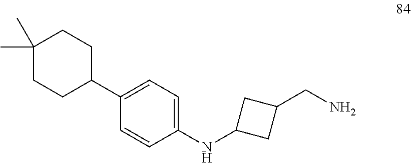 [SMILES: CC1(C)CCC(c2ccc(NC3CC(CN)C3)cc2)CC1]

The title compound 84 (94 mg, 78.2%) was prepared as an off-white solid with 1:0.43 mixture by ¹H NMR from 3-((4-(4,4-dimethylcyclohexyl)phenyl)amino)cyclobutane-1-carboxamide, THF, and AlLiH₄ according to the procedure for 23. ¹H NMR (400 MHZ, DMSO-d₆) δ 7.89 (s, 4H), 6.94 (d, J=8.3 Hz, 3H), 6.45-6.36 (m, 3H), 5.68 (dd, J=6.6, 0.0 Hz, 1H), 5.59 (d, J=7.1 Hz, 0.43H), 3.89 (h, J=7.0 Hz, 1H), 3.67 (q, J=7.4 Hz, 0.43H), 2.96 (d, J=7.9 Hz, 2H), 2.82 (d, J=7.3 Hz, 1H), 2.49-2.38 (m, 3H), 2.21 (td, J=11.9, 5.5 Hz, 4H), 2.09-1.85 (m, 3H), 1.58-1.46 (m, 7H), 1.42 (d, J=13.8 Hz, 4H), 1.33-1.20 (m, 6H), 0.93 (d, J=8.3 Hz, 9H). Mass (m/z): 287.3 [M+H]⁺.

### Compound 85

### 4-(aminomethyl)-N-(4-(4,4-dimethylcyclohexyl)phenyl)cycloheptan-1-amine

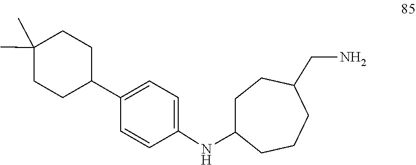 [SMILES: CC1(C)CCC(c2ccc(NC3CCCC(CN)CC3)cc2)CC1]

The title compound 85 (15.8 mg) was prepared in a three-step overall yield of 9.78% as a white powder with 1:1 mixture by ¹H NMR from 4-(4,4-dimethylcyclohexyl)aniline (100 mg, 0.49 mmol), 4-oxocycloheptane-1-carboxylic acid (92 mg, 0.59 mmol) according to the procedure for 23. ¹H NMR (400 MHZ, DMSO-d₆) δ 7.73 (s, 3H), 7.20 (s, 2H), 6.94 (s, 1H), 2.70-2.58 (m, 2H), 2.35 (s, 1H), 2.02-1.90 (m, 1H), 1.83 (s, 1H), 1.74 (d, J=14.5 Hz, 3H), 1.63-1.48 (m, 7H), 1.47-1.37 (m, 3H), 1.29 (td, J=12.6, 6.3 Hz, 3H), 1.10 (t, J=13.6 Hz, 1H), 0.95 (s, 3H), 0.93 (s, 3H). Mass (m/z): 329.6 [M+H]⁺.

### Compound 86

### 4-(4,4-dimethylcyclohexyl)-N-(4-((methylamino)methyl)cyclohexyl)aniline

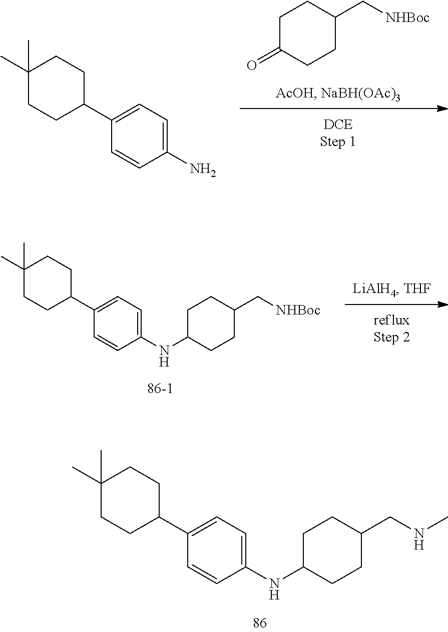

Step 1. Preparation of tert-butyl ((4-((4-(4,4-dimethylcyclohexyl)phenyl)amino)cyclohexyl)methyl)carbamate(86-1)

A mixture of 4-(4,4-dimethylcyclohexyl)aniline (101.5 mg, 0.5 mmol), tert-butyl ((4-oxocyclohexyl)methyl)carbamate (136 mg, 0.6 mmol) and acetic acid (0.029 ml, 0.5 mmol) in DCE (5 mL) was stirred for 1 h at room temperature. Afterwards 211 mg (1 mmol) of sodium triacetoxyborohydride were added and the mixture was stirred overnight. To the mixture, saturated NaHCO₃aq. was added and the mixture was extracted with ethyl acetate. The combined organic layers were dried over MgSO₄ and evaporated. The mixture was purified by prep-TLC to afford the intermediate (176 mg, 85%) as a white solid. Mass (m/z): 415.2 [M+H]⁺.

Step 2. Preparation of 4-(4,4-dimethylcyclohexyl)-N-(4-((methylamino)methyl)cyclohexyl)aniline (86)

tert-butyl ((4-((4-(4,4-dimethylcyclohexyl)phenyl)amino)cyclohexyl)methyl)carbamate(41 mg, 0.1 mmol) in THF (1 mL) was slowly added dropwise to a suspension of LiAlH₄ (14.8 mg, 0.4 mmol) in THF (1 mL). The mixture was heated to reflux and stirred overnight. To the mixture, saturated NaHCO₃aq. was added and the mixture was extracted with ethyl acetate. The combined organic layers were dried over MgSO₄ and evaporated. The mixture was purified by prep-TLC to afford the title compounds (16.2 mg, 49%) as a rosy brown solid with 1:2 mixture by ¹H NMR. ¹H NMR (400 MHZ, DMSO-d₆) δ 8.92 (s, 2H), 6.94 (d, J=8.8 Hz, 2H), 6.54 (d, J=8.8 Hz, 2H), 3.41 (s, 1H), 3.36 (s, 3H), 2.78 (m, 1.3H), 2.72 (m, 0.6H), 2.20 (m, 1H), 1.97 (m, 0.6H), 1.81 (m, 1.3H), 1.68-1.35 (m, 12H), 1.26 (m, 2H), 1.07 (m, 1H), 0.93 (s, 3H), 0.91 (s, 3H). Mass (m/z): 329.3 [M+H]⁺.

### Compound 87

### 4-(tert-butyl)-N-(4-((methylamino)methyl)cyclohexyl)aniline

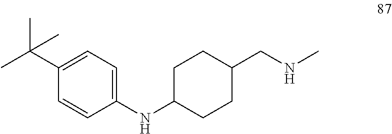 [SMILES: CNCC1CCC(Nc2ccc(C(C)(C)C)cc2)CC1]

The title compound 87 (16.4 mg) was prepared in a total yield of 60% as a yellow solid with 1:3 mixture by ¹H NMR from tert-butyl ((4-((4-(tert-butyl)phenyl)amino)cyclohexyl)methyl)carbamate(36 mg, 0.1 mmol), and LiAlH₄ (14.8 mg, 0.4 mmol) according to the procedure for 86. ¹H NMR (400 MHz, Methanol-d₄) δ 7.35-7.15 (m, 2H), 6.87-6.66 (m, 2H), 3.55 (m, 0.7H), 3.22 (m, 0.3H), 2.96 (d, J=6.8 Hz, 1.5H), 2.89 (d, J=6.4 Hz, 0.5H), 2.71 (s, 3H), 2.16-1.45 (m, 8H), 1.25 (s, 9H). Mass (m/z): 275.4 [M+H]⁺.

### Compound 88

### N-(4-(aminomethyl)cyclohexyl)-4-(trifluoromethyl)aniline

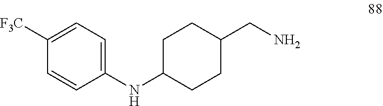 [SMILES: NCC1CCC(Nc2ccc(C(F)(F)F)cc2)CC1]

The desired product as a white solid (35 mg, 65%) with 1:2 mixture by ¹H NMR was prepared from tert-butyl ((4-((4-(trifluoromethyl)phenyl)amino)cyclohexyl)methyl)carbamate(74 mg, 0.2 mmol) and TFA (0.15 mL, 2 mmol) according to the procedure for 24. ¹H NMR (400 MHZ, DMSO-d₆) δ 7.82 (s, 3H), 7.34-7.29 (m, 2H), 6.69-6.58 (m, 2H), 3.50 (m, 0.7H), 3.16 (m, 0.3H), 2.68 (d, J=6.0 Hz, 1.4H), 2.64 (d, J=6.8 Hz, 0.7H), 1.96 (m, 1H), 1.83-1.07 (m, 8H). Mass (m/z): 273.3 [M+H]⁺.

### Compound 89

### 4-((4-(4,4-dimethylcyclohexyl)phenyl)amino)-N,N-dimethylcyclohexane-1-carboxamide

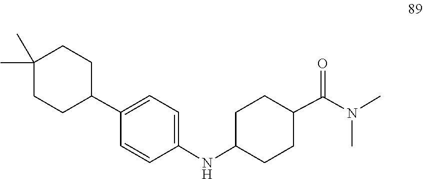

The title compound 89 (5.4 mg) was prepared in a total yield of 14% as a white solid with 1:3 mixture by ¹H NMR from 4-((4-(4,4-dimethylcyclohexyl)phenyl)amino)cyclohexane-1-carboxylic acid (33 mg, 0.1 mmol), dimethylamine (0.45 mg, 0.15 mmol), DIEA (0.045 mL, 0.26 mmol) and DMT-MM (76 mg, 0.26 mmol) according to the procedure for 1. ¹H NMR (400 MHZ, DMSO-d₆) δ 6.92 (d, J=8.8 Hz, 2H), 6.52 (d, J=8.8 Hz, 0.7H), 6.48 (d, J=8.8 Hz, 1.3H), 3.44 (m, 1H), 3.09 (m, 1H), 3.01 (s, 3H), 2.80 (s, 3H), 1.99 (m, 1H), 1.72-1.36 (m, 12H), 1.33-1.12 (m, 4H), 0.94 (s, 3H), 0.92 (s, 3H). Mass (m/z): 357.2 [M+H]⁺.

### Compound 90

### N-hydroxy-2-(4-methylpiperazin-1-yl)-N-(4-((4-(1-methylpiperidin-4-yl)phenyl)amino)benzyl)a cetamide

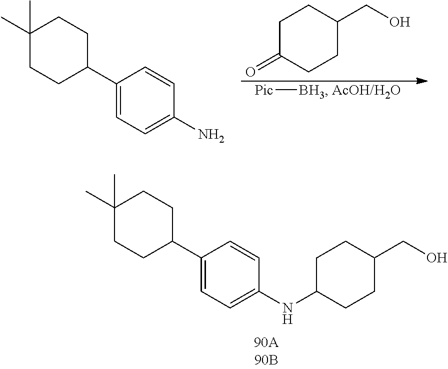

A solution of 4-(4,4-dimethylcyclohexyl)aniline (100 mg, 0.49 mmol), 4-(hydroxymethyl) cyclohexan-1-one (94.55 mg, 0.7377 mmol) and Pic-BH₃ (52-picoline-borane; 3 mg, 0.49 mmol), in AcOH/H₂O (10/1) (22 mL) was stirred 2 hrs at 25° C. After filtration, the solvent was removed under vacuum and the residue was purified by prep-HPLC (column-Xbridge-C18 150×21.2 mm, 5 μm; Mobile phase: ACN-H₂O (0.1% FA), 40%-60%) to afford 90A (94 mg) as a white solid and 90B (86 mg) as a white solid. 90A: ¹H NMR (300 MHZ, DMSO-d₆) δ 6.92 (d, J=8.4 Hz, 2H), 6.47 (d, J=8.4 Hz, 2H), 5.11 (s, 1H), 4.42 (s, 1H), 3.23 (d, J=5.3 Hz, 2H), 3.06 (s, 1H), 2.21 (s, 2H), 1.98 (d, J=10.6 Hz, 1H), 1.76 (d, J=11.6 Hz, 2H), 1.54-1.31 (m, 9H), 1.11-0.95 (m, 4H), 0.93 (d, J=8.4 Hz, 6H). Mass (m/z): 316.3 [M+H]⁺. HPLC: Rt=5.824 min (column-Xbridge 5u C18 150×19 mm; Flow Rate: 20 mL/min. Mobile phase: ACN-H₂O (0.1% FA), 35%-60%). 90B: MS (m/z): 316.3 [M+H]⁺. ¹H NMR (400 MHZ, DMSO-d₆) δ 6.87 (d, J=8.4 Hz, 2H), 6.46 (d, J=8.4 Hz, 2H), 5.16-5.04 (m, 1H), 4.38-4.30 (m, 1H), 3.42-3.34 (m, 1H), 3.23 (d, J=5.2 Hz, 2H), 2.22-2.07 (m, 1H), 1.60-1.17 (m, 18H), 0.89 (d, J=8.4 Hz, 6H). MS (m/z): 316.3 [M+H]⁺. HPLC Rt=7.933 min (column-Xbridge 5u C18 150×19 mm; Flow Rate: 20 mL/min. Mobile phase: ACN-H₂O (0.1% FA), 35%-60%).

### Compound 91

### N1-(4-(4,4-dimethylcyclohexyl)phenyl)-N4,N4-dimethylcyclohexane-1,4-diamine

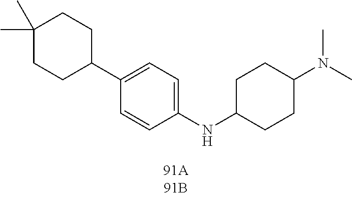

The title compound 91A, 91B was prepared according to the procedure for compound 1-1. The crude residue was purified by preparative TLC (H₂O/MeOH/DCM=0.1/1/5) to afford compound 91A (R_{f} value=0.36) in 19.2% yield as a white solid and 91B (R_{f} value=0.30) in 14.8% yield as a white solid. 91A: ¹H NMR (400 MHz, DMSO-d₆) δ 10.10 (s, 1H), 6.94-6.87 (m, 2H91), 6.51 (d, J=8.6 Hz, 2H), 3.55-3.46 (m, 1H), 3.17-3.06 (m, 1H), 2.65 (s, 6H), 2.23-2.14 (m, 1H), 1.88-1.81 (m, 2H), 1.77-1.66 (m, 4H), 1.57-1.46 (m, 6H), 1.41-1.35 (m, 2H), 1.27-1.19 (m, 2H), 0.89 (d, J=8.6 Hz, 6H). Mass (m/z): 329.3 [M+H]⁺. 91B: ¹H NMR (400 MHz, DMSO-d₆) δ 6.90 (d, J=8.0 Hz, 2H), 6.44 (d, J=8.0 Hz, 2H), 5.19 (s, 1H), 3.10 (t, J=11.2 Hz, 2H), 2.65 (t, J=4.8 Hz, 6H), 2.22-2.12 (m, 1H), 2.04-1.97 (m, 4H), 1.57-1.36 (m, 8H), 1.26-1.08 (m, 4H), 0.89 (d, J=8.4 Hz, 6H). Mass (m/z): 329.3 [M+H]⁺

### Compound 92

### N-(4-(aminomethyl)cyclohexyl)aniline

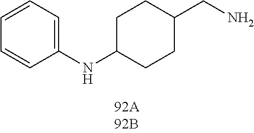 [SMILES: NCC1CCC(Nc2ccccc2)CC1]

The desired products 92A (Rt=7.1 min) as yellow oil (122.2 mg, 67.4%) and 92B (Rt=7.9 min) as a white solid (45.5 mg, 25.1%) were prepared from tert-butyl ((4-(phenylamino)cyclohexyl)methyl)carbamate (270 mg, 0.888 mmol), DCM (10 mL) and TFA (1 mL) according to the procedure for 24, which were purified by Prep-HPLC (Column: X Select-CSH-Prep 5 μm OBD, 19*150 mm; solvent system (ACN/water (0.5% TFA)=0-90%-50%-50%-100%, 0-10 min-10.5 min-11.5 min-13 min). 92A: ¹H NMR (400 MHZ, DMSO-d₆) δ 7.91 (s, 3H), 7.21 (t, J=7.6 Hz, 2H), 6.91 (d, J=8.0 Hz, 2H), 6.84 (t, J=7.6 Hz, 1H), 3.45 (tt, J=6.8, 3.7 Hz, 1H), 2.74 (p, J=6.0 Hz, 2H), 1.73 (h, J=6.0 Hz, 1H), 1.67-1.43 (m, 8H). Mass (m/z): 205.3 [M+H]⁺. 92B: ¹H NMR (400 MHZ, DMSO-d₆) δ 7.82 (s, 3H), 7.21 (t, J=7.7 Hz, 2H), 6.96-6.79 (m, 3H), 3.21 (tt, J=11.3, 3.8 Hz, 1H), 2.64 (p, J=5.8 Hz, 2H), 1.99-1.88 (m, 2H), 1.84-1.72 (m, 2H), 1.50 (ttt, J=10.5, 6.9, 3.4 Hz, 1H), 1.28-1.12 (m, 2H), 1.00 (qd, J=13.2, 3.2 Hz, 2H). Mass (m/z): 205.3 [M+H]⁺.

### Compound 93

### N-(4-(4,4-dimethylcyclohexyl)phenyl)tetrahydro-2H-pyran-4-amine

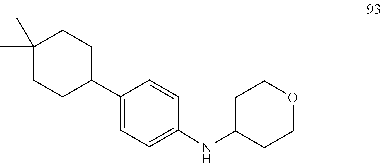 [SMILES: CC1(C)CCC(c2ccc(NC3CCOCC3)cc2)CC1]

The title compound 93 (28.5 mg) was prepared in a yield of 67.2% as a white powder from 4-(4,4-dimethylcyclohexyl)aniline (30 mg, 0.14 mmol), tetrahydro-4H-pyran-4-one (29 mg, 0.29 mmol) and according to the procedure for 4. ¹H NMR (400 MHZ, DMSO-d₆) δ 6.92 (d, J=8.3 Hz, 2H), 6.51 (d, J=8.1 Hz, 2H), 5.23 (d, J=8.2 Hz, 1H), 3.95-3.75 (m, 2H), 3.41-3.37 (m, 2H), 2.20 (td, J=10.3, 5.3 Hz, 1H), 1.93-1.78 (m, 2H), 1.52 (dd, J=9.4, 4.4 Hz, 3H), 1.48-1.36 (m, 3H), 1.35-1.21 (m, 5H), 0.94 (s, 3H), 0.92 (s, 3H). Mass (m/z): 289.6 [M+H]⁺.

### Compound 94

### N1-(4-(4,4-dimethylcyclohexyl)phenyl)cyclobutane-1,3-diamine

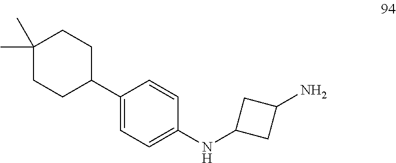 [SMILES: CC1(C)CCC(c2ccc(NC3CC(N)C3)cc2)CC1]

The title compound 94 (46.3 mg) was prepared in a yield of 88.6% as a pale yellow powder with 1:0.1 mixture by ¹H NMR from 4-(4,4-dimethylcyclohexyl)aniline (39 mg, 0.19 mmol), and tert-butyl (3-oxocyclobutyl)carbamate (53 mg, 0.29 mmol) according to the procedure for 20. ¹H NMR (400 MHZ, DMSO-d₆) δ 8.36 (s, 3H), 7.00-6.88 (m, 2H), 6.45-6.41 (m, 0.2H), 6.41-6.34 (m, 2H), 5.81 (d, J=6.2 Hz, 1H), 5.73 (d, J=6.4 Hz, 0.1H), 4.05 (q, J=7.0 Hz, 1H), 3.72 (tt, J=8.1, 5.2 Hz, 1H), 2.44 (tt, J=7.7, 5.2 Hz, 2H), 2.29-2.09 (m, 3H), 1.51 (dp, J=11.7, 3.8 Hz, 4H), 1.41 (dt, J=14.5, 3.1 Hz, 2H), 1.34-1.18 (m, 3H), 0.93 (s, 3H), 0.91 (s, 3H). Mass (m/z): 273.4 [M+H]⁺.

### Compound 95

### N⁴-(4-(tert-butyl)phenyl)adamantane-1,4-diamine

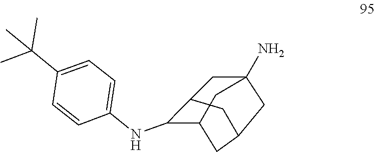 [SMILES: CC(C)(C)c1ccc(NC2C3CC4CC2CC(N)(C4)C3)cc1]

The title compound 95 (25.6 mg) was prepared in a total yield of 36.6% as a white solid according to the procedure for 20. ¹H NMR (400 MHZ, DMSO-d₆) δ 8.02 (s, 2H), 7.94 (s, 1H), 7.12-7.04 (m, 2H), 6.55 (dd, J=20.0, 8.4 Hz, 2H), 5.40-5.27 (m, 1H), 2.23-2.06 (m, 2H), 2.05-1.75 (m, 7H), 1.72-1.63 (m, 1H), 1.63-1.51 (m, 1H), 1.39-1.29 (m, 2H), 1.20 (s, 9H). Mass (m/z): 299.3 [M+H]⁺.

### Compound 96

### N-(4-(aminomethyl)cyclohexyl)-4-(tert-butyl)aniline

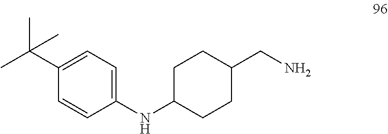 [SMILES: CC(C)(C)c1ccc(NC2CCC(CN)CC2)cc1]

The title compound 96 (4.8 mg) was prepared in a total yield of 19% as a white solid with 1:2 mixture by ¹H NMR from tert-butyl ((4-((4-(tert-butyl)phenyl)amino)cyclohexyl)methyl)carbamate(36 mg, 0.1 mmol), and TFA (0.075 mL, 1 mmol) according to the procedure for 24. ¹H NMR (400 MHz, DMSO-d₆) δ 7.93 (s, 3H), 7.07 (d, J=8.0 Hz, 2H), 6.47 (d, J=8.0 Hz, 2H), 3.40 (m, 0.7H), 3.06 (m, 0.3H), 2.63 (m, 2H), 1.95 (m, 0.6H), 1.77 (m, 0.3H), 1.68-1.32 (m, 8H), 1.17 (s, 9H). Mass (m/z): 261.2 [M+H]⁺.

### Compound 97

### N-(4-((dimethylamino)methyl)cyclohexyl)-4-(4,4-dimethylcyclohexyl)aniline

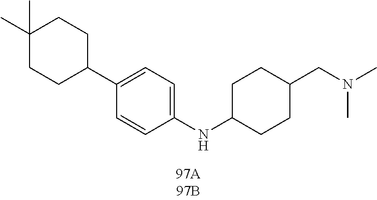

The title compounds 97A (Rt=5.48 min; 4.8 mg, 28%) and 97B (Rt=6.22 min; 2.0 mg, 12%) were prepared from 4-((4-(4,4-dimethylcyclohexyl)phenyl)amino)-N,N-dimethylcyclohexane-1-carboxamide (36 mg, 0.1 mmol), and LiAlH₄ (14.8 mg, 0.4 mmol) according to the procedure for 23, which were purified by preparative HPLC (Column: X Select-CSH-Prep 5 μm OBD, 19*150 mm; ACN/water (5% % TFA=20%-65%-95%-95%-10%, 0-13 min-13.5 min-14.5 min-16.0 min). 97A: ¹H NMR (400 MHZ, DMSO-d₆) δ 6.92 (d, J=8.4 Hz, 2H), 6.46 (d, J=8.4 Hz, 2H), 3.06 (m, 1H), 2.88 (m, 2H), 2.69 (s, 6H), 2.20 (m, 1H), 1.98-1.82 (m, 5H), 1.54-1.38 (m, 8H), 1.14-1.02 (m, 4H), 0.90 (s, 3H), 0.88 (s, 3H). Mass (m/z): 343.4 [M+H]. 97B: ¹H NMR (400 MHz, DMSO-d₆) δ 6.92 (d, J=8.4 Hz, 2H), 6.50 (d, J=8.4 Hz, 2H), 2.73 (m, 1H), 2.67 (m, 2H), 2.51 (s, 6H), 2.20 (m, 1H), 1.94-1.03 (m, 4H), 1.65-1.35 (m, 13H), 0.94 (s, 3H), 0.92 (s, 3H). Mass (m/z): 343.4 [M+H]⁺.

### Compound 98

### 5-(aminomethyl)-N-(4-(tert-butyl)phenyl)adamantan-2-amine

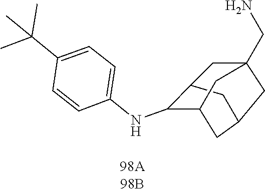 [SMILES: CC(C)(C)c1ccc(NC2C3CC4CC2CC(CN)(C4)C3)cc1]

The title compound 98A and 98B were prepared according to the procedure for compound 20. The product was purified by preparative HPLC (Column: X Select-CSH-Prep 5 μm OBD, 19*150 mm; ACN/water (0.5% TFA)=0%-0%-30%-95%-95%-0%, 0 min-2 min-9 min-9.5 min-10.5 min-12.0) to afford compound 98A (Rt=9.03 min) in 35.6% yield as a white solid and 98B (Rt=7.56 min) in 31.2% yield as a white solid. 98A ¹H NMR (400 MHZ, Methanol-d₄) δ 7.17-7.11 (m, 2H), 6.66-6.60 (m, 2H), 3.49-3.42 (m, 1H), 2.33 (s, 2H), 2.08-2.01 (m, 4H), 1.97-1.90 (m, 1H), 1.64-1.60 (m, 4H), 1.55-1.51 (m, 2H), 1.51-1.44 (m, 1H), 1.25 (s, 9H). Mass (m/z): 313.3 [M+H]⁺. 98B ¹H NMR (400 MHZ, Methanol-d₄) δ 7.17-7.11 (m, 2H), 6.67-6.60 (m, 2H), 3.51-3.46 (m, 1H), 2.42 (s, 2H), 2.13-2.07 (m, 2H), 2.03-1.97 (m, 1H), 1.88-1.78 (m, 5H), 1.54-1.47 (m, 2H), 1.35-1.27 (m, 2H), 1.25 (s, 9H). Mass (m/z): 313.3 [M+H]⁺.

### Compound 99

### N1-(2,3-dihydro-1H-inden-5-yl)-4-methylcyclohexane-1,4-diamine

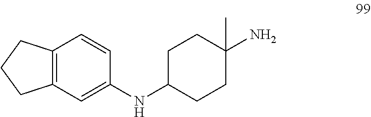 [SMILES: CC1(N)CCC(Nc2ccc3c(c2)CCC3)CC1]

The title compound 99 (102.5 mg) was prepared in a total yield of 93.8% as a white solid from tert-butyl (4-((2,3-dihydro-1H-inden-5-yl)amino)-1-methylcyclohexyl)carbamate (168 mg, 0.488 mmol), TFA (1 mL), and DCM (10 mL) according to the procedure for 24. ¹H NMR (400 MHZ, DMSO-d₆) δ 8.29 (d, J=36.0 Hz, 3H), 6.90 (d, J=8.0 Hz, 1H), 6.53-6.45 (m, 1H), 6.44-6.34 (m, 1H), 5.34-4.83 (m, 1H), 3.30-3.08 (m, 1H), 2.70 (dt, J=15.2, 7.2 Hz, 4H), 1.99-1.87 (m, 4H), 1.80-1.48 (m, 6H), 1.30 (d, J=8.0 Hz, 3H). Mass (m/z): 245.3 [M+H]⁺.

### Compound 100

### N-(4-(4,4-dimethylcyclohexyl)phenyl)-1′-methyl-[1,4′-bipiperidin]-4-amine

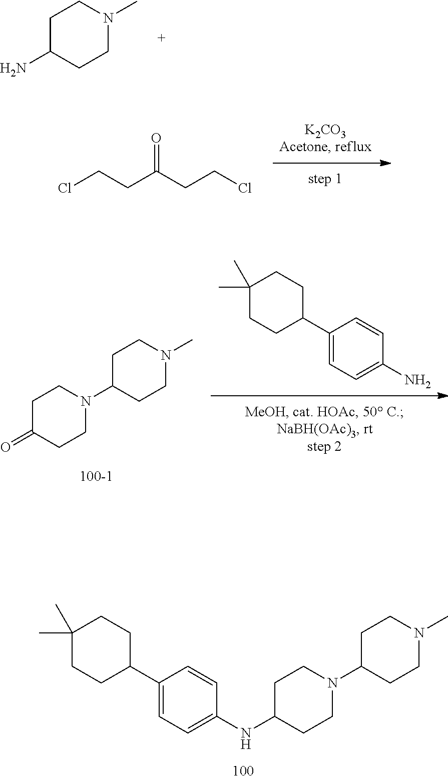

Step 1. Preparation of 1′-methyl-[1,4′-bipiperidin]-4-one (100-1) To a solution of 1-methylpiperidin-4-amine (1 g, 8.8 mmol) in acetone (20 mL) was added K₂CO₃ (2.7 g, 19.3 mmol) and 1,5-dichloropentan-3-one (1.5 g, 9.7 mmol) at 25° C. Then the mixture was stirred at 70° C. for 3 hrs. LCMS showed the reaction was completed. The mixture was filtered and concentrated to give compound 2 (0.5 g, 27% yield) as yellow oil. MS (m/z): 197.1 [M+H]⁺.

Step 2. Preparation of N-(4-(4,4-dimethylcyclohexyl)phenyl)-1′-methyl-[1,4′-bipi peridin]-4-amine (100)

To a solution of compound 100-1 (0.5 g, 2.55 mmol) in MeOH (20 mL) and a drop of AcOH was added 4-(4,4-dimethylcyclohexyl)aniline (570 mg, 2.8 mmol) at 25° C. Then the mixture was stirred at 50° C. for 1 h. Then the mixture was added NaBH₃CN (480 mg, 7.6 mmol) after cooling to 25° C. Then the mixture was stirred at 25° C. for 2 hrs. LCMS showed the reaction was completed. The reaction was quenched with water (10 mL), extracted with EA (10 mL*3). The combined organic layers were washed with brine (20 mL), dried over sodium sulfate, filtered and concentrated. The residue was purified by combi-flash with EA/PE (1:0) to afford to give compound 5 (10 mg, 10.2% yield) as a yellow solid. ¹H NMR (300 MHZ, CD₃OD) δ 6.88 (d, J=8.1 Hz, 2H), 6.52 (d, J=8.1 Hz, 2H), 3.25 (s, 3H), 2.97-2.85 (m, 4H), 2.33 (t, J=11.5 Hz, 3H), 2.06-1.81 (m, 7H), 1.55-1.45 (m, 7H), 1.44-1.30 (m, 6H), 0.86 (d, J=12.1 Hz, 6H). MS (m/z) 384.0 [M+H]⁺.

### Compound 101

### 4-(4,4-dimethylcyclohexyl)-N-(2-(pyrrolidin-1-yl)ethyl)aniline

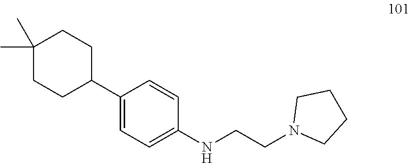 [SMILES: CC1(C)CCC(c2ccc(NCCN3CCCC3)cc2)CC1]

The title compound 101 (8 mg, 9.0%) was prepared from 2-(pyrrolidin-1-yl) acetaldehyde (40 mg, 0.35 mmol), 4-(4,4-dimethylcyclohexyl)aniline (60 mg, 0.30 mmol), MeOH (10 mL), 1 drop of AcOH, and NaBH₃CN (14.2 mg, 0.89 mmol) according to the procedure for 5. H NMR (400 MHZ, CDCl₃) δ 8.56 (s, 1H), 7.05 (d, J=8.4 Hz, 2H), 6.57 (d, J=8.4 Hz, 2H), 3.52 (t, J=5.6 Hz, 2H), 3.19 (t, J=5.6 Hz, 6H), 2.32-2.26 (m, 1H), 2.02 (t, J=6.6 Hz, 4H), 1.69-1.42 (m, 6H), 1.30 (td, J=13.1, 4.1 Hz, 2H), 0.96 (t, J=7.9 Hz, 6H). Mass (m/z): 301.0 [M+H]⁺.

### Compound 102

### 4-((4-(4,4-dimethylcyclohexyl)phenyl)amino)piperidin-2-one

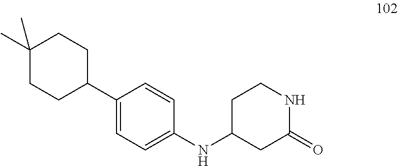 [SMILES: CC1(C)CCC(c2ccc(NC3CCNC(=O)C3)cc2)CC1]

The title product 102 (42 mg, 56%) as a yellow solid was prepared from 4-(4,4-dimethylcyclohexyl)aniline (50 mg, 0.25 mmol), MeOH (5 mL), piperidine-2,4-dione (30 mg, 0.27 mmol), acetic acid (1.5 mg, 0.025 mmol) and sodium cyanoborohydride (46 mg, 0.75 mmol) according to the procedure for 5. ¹H NMR (300 MHz, CDCl₃) δ 7.08 (d, J=8.4 Hz, 2H), 6.62 (d, J=8.4 Hz, 2H), 6.05 (s, 1H), 3.85-3.76 (m, 1H), 3.49-3.31 (m, 2H), 2.83 (dd, J=17.4 Hz, 4.5 Hz, 1H), 2.36-2.27 (m, 2H), 2.20-2.16 (m, 1H), 1.85-1.73 (m, 2H), 1.67-1.63 (m, 1H), 1.58-1.54 (m, 1H), 1.50-1.45 (m, 2H), 1.36-1.25 (m, 2H), 0.95 (d, J=5.1 Hz, 6H). Mass (m/z): 301.2 [M+H]⁺.

### Compound 103

### 4-((4-(4,4-dimethylcyclohexyl)phenyl)amino)-1-methylpiperidin-2-one

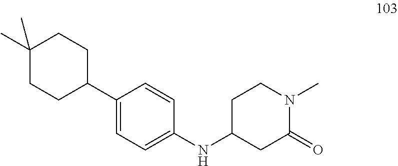

The desired product (50 mg, 63.7%) as a white solid was obtained from 4-(4,4-dimethylcyclohexyl)aniline (50 mg, 0.25 mmol), MeOH (5 mL), 1-methylpiperidine-2,4-dione (35 mg, 0.27 mmol), acetic acid (1.5 mg, 0.025 mmol) and sodium cyanoborohydride (46 mg, 0.75 mmol) according to the procedure for 5. ¹H NMR (400 MHz, CDCl₃) δ 7.06 (d, J=8.4 Hz, 2H), 6.58 (d, J=8.4 Hz, 2H), 3.79-3.75 (m, 1H), 3.40-3.33 (m, 2H), 2.97 (s, 3H), 2.84 (dd, J=17.2 Hz, 4.4 Hz, 1H), 2.33-2.27 (m, 2H), 2.22-2.19 (m, 1H), 1.84-1.80 (m, 2H), 1.66-1.60 (m, 1H), 1.57-1.54 (m, 1H), 1.49-1.44 (m, 2H), 1.31 (td, J=8.8 Hz, 4.0 Hz, 2H), 0.95 (d, J=7.2 Hz, 6H). Mass (m/z): 315.2 [M+H]⁺.

### Compound 104

### N-hydroxy-2-(4-methylpiperazin-1-yl)-N-(4-((4-(1-methylpiperidin-4-yl)phenyl)amino)benzyl)a cetamide

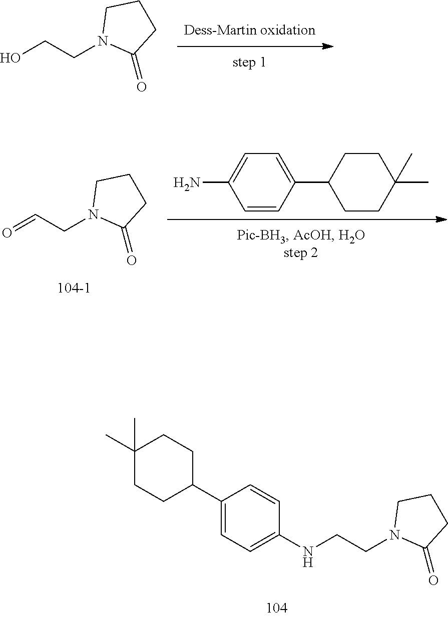 [SMILES: O=C1CCCN1CCO ; O=CCN1CCCC1=O ; CC1(C)CCC(c2ccc(N)cc2)CC1 ; CC1(C)CCC(c2ccc(NCCN3CCCC3=O)cc2)CC1]

Step 1. Preparation of 2-(2-oxopyrrolidin-1-yl) acetaldehyde (104-1)

A mixture of 1-(2-hydroxyethyl) pyrrolidin-2-one (2 g, 15.4 mmol) and Dess-Martin reagent (13.06 g, 30.8 mmol in DCM (50 mL) was stirred for 10 mins at rt. The reaction mixture was filtered and evaporated, and the residue was purified by silica gel column (PE/EA=1:1) to afford the product as yellow oil. (1.54 g, 78.2%).

Step 2. Preparation of 1-(2-((4-(4,4-dimethylcyclohexyl)phenyl)amino)ethyl) pyrrolidin-2-one (104)

A mixture solution of 2-(2-oxopyrrolidin-1-yl) acetaldehyde (50 mg, 0.35 mmol), 4-(4,4-dimethylcyclohexyl)aniline (60 mg, 0.3 mmol) and Pic-BH₃ (56 mg, 0.88 mmol) in AcOH/H₂O=1/9 (5 mL) was stirred 3 hours at rt. The reaction mixture was adjusted to pH 8 with aqueous saturated NaHCO₃, exacted with EA (10 mL), dried over Na₂SO₄, filtered and concentrated under vacuum. The residue was purified by prep-HPLC to afford the desired product (10 mg, 6.7%) as a white solid). ¹H NMR (400 MHZ, CDCl₃) δ 7.07 (d, J=8.4 Hz, 2H), 6.68 (d, J=8.4 Hz, 2H), 3.58 (t, J=5.7 Hz, 2H), 3.44 (t, J=7.1 Hz, 2H), 3.35-3.27 (m, 2H), 2.39 (t, J=8.1 Hz, 2H), 2.36-2.26 (m, 1H), 2.01 (dt, J=15.5, 7.6 Hz, 2H), 1.69-1.43 (m, 7H), 1.36-1.23 (m, 3H), 0.95 (d, J=7.1 Hz, 6H). Mass (m/z): 314.7 [M+H]⁺.

### Compound 105

### 4-(4,4-dimethylcyclohexyl)-N-(4-(pyrrolidin-1-yl)butyl)aniline

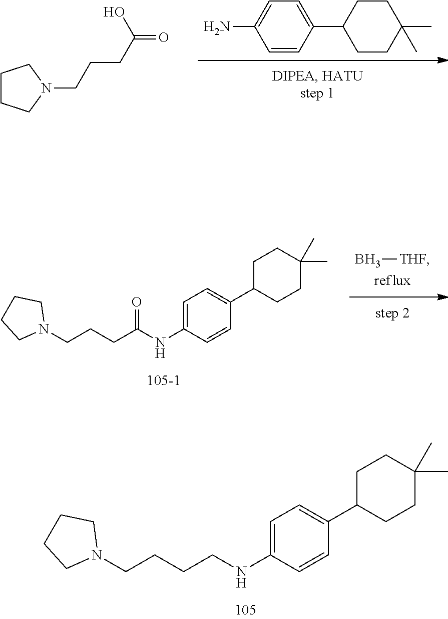

Step 1. Preparation of N-(4-(4,4-dimethylcyclohexyl)phenyl)-4-(pyrrolidin-1-yl) butanamide (105-1)

To a solution of 4-(pyrrolidin-1-yl) butanoic acid (46 mg, 0.29 mmol) in DCM (5 ml) was added 4-(4,4-dimethylcyclohexyl)aniline (50 mg, 0.25 mmol), DIEA (95 mg, 0.75 mmol) and HATU (140 mg, 0.375 mmol). Then the mixture was stirred at r.t. for 3 h. The reaction was extracted by EA (10 mL) for 3 times. The combined organic layers were dried over sodium sulfate and concentrated under vacuum. The residue was purified by flash to afford the desired product (35 mg, 40.9%) as yellow oil. Mass (m/z): 330.3 [M+H]⁺.

Step 2. Preparation of 4-(4,4-dimethylcyclohexyl)-N-(4-(pyrrolidin-1-yl)butyl)aniline (105)

To a solution of N-(4-(4,4-dimethylcyclohexyl)phenyl)-4-(pyrrolidin-1-yl) butanamide (35 mg, 0.10 mmol) in THF (2 mL) was added BH₃-THF (15 mL) and the mixture was stirred at 70° C. for 2 h. The reaction was quenched with water (10 mL), and extracted by EA (10 mL) for 3 times. The combined organic phase layers were dried over sodium sulfate and concentrated under vacuum. The residue was purified by prep-HPLC (column-Xbridge-C18 150×21.2 mm, 5 μm; Mobile phase: ACN-H₂O (0.1% FA), 40%-60%) to afford the desired product (13 mg, 41.2%) as a black solid. ¹H NMR (400 MHZ, CDCl₃) δ 7.04 (d, J=8.4 Hz, 2H), 6.57 (d, J=8.4 Hz, 2H), 3.69 (t, J=5.6 Hz, 1H), 3.15 (t, J=6.8 Hz, 2H), 2.91-2.82 (m, 2H), 2.30 (m, 1H), 2.05-1.96 (m, 4H), 1.91-1.83 (m, 1H), 1.73-1.66 (m, 2H), 1.56 (dd, J=12.4 Hz, 3.2 Hz, 3H), 1.46 (d, J=12.6, 2H), 1.32 (dd, J=13.1 Hz, 4.0 Hz, 3H), 0.95 (d, J=7.4 Hz, 6H). Mass (m/z): 329.3 [M+H]⁺.

### Compound 106

### N-(4-((4-(4,4-dimethylcyclohexyl)phenyl)amino)cyclohexyl)methyl)-5-oxopyrrolidine-3-carbo xamide

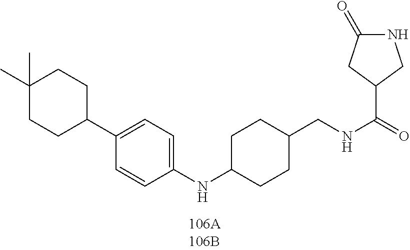 [SMILES: CC1(C)CCC(c2ccc(NC3CCC(CNC(=O)C4CNC(=O)C4)CC3)cc2)CC1]

The title compounds 106A (Rt=5.28 min; 6.8 mg) in a total yield of 27% as a white solid and 106B (Rt=6.21 min; 5.4 mg) in a total yield of 21% as a white solid were prepared from N-(4-(aminomethyl)cyclohexyl)-4-(4,4-dimethylcyclohexyl)aniline (19 mg, 0.06 mmol) according to the procedure for 1, which were purified by preparative HPLC (Column: X Select-CSH-Prep 5 μm OBD, 19*150 mm; ACN/water (5% % TFA)=30%-45%-75%-95%-10%, 0-7 min-7.5 min-8.5 min-10.0 min). 106A: ¹H NMR (400 MHZ, DMSO-d₆) δ 7.97 (t, J=5.6 Hz, 1H), 7.57 (s, 1H), 6.92 (d, J=8.4 Hz, 2H), 6.47 (d, J=8.4 Hz, 2H), 5.14 (m, 1H), 3.38 (m, 2H), 3.23-3.08 (m, 2H), 3.00 (t, J=6.4 Hz, 2H), 2.31-2.17 (m, 3H), 1.96 (m, 1H), 1.70 (m, 1H), 1.65-1.26 (m, 15H), 0.94 (s, 3H), 0.92 (s, 3H). Mass (m/z): 426.3 [M+H]⁺. 106B: ¹H NMR (400 MHZ, DMSO-d₆) δ 7.97 (t, J=5.6 Hz, 1H), 7.57 (s, 1H), 6.92 (d, J=8.4 Hz, 2H), 6.51 (d, J=8.4 Hz, 2H), 5.17 (m, 1H), 3.41 (m, 2H), 3.21-3.06 (m, 2H), 2.93 (t, J=6.4 Hz, 2H), 2.33-2.17 (m, 3H), 2.06-1.31 (m, 17H), 0.94 (s, 3H), 0.92 (s, 3H). Mass (m/z): 426.3 [M+H]⁺.

### Compound 107

### (S)—N-((4-((4-(4,4-dimethylcyclohexyl)phenyl)amino)cyclohexyl)methyl)-2-oxoimidazolidine-4-carboxamide

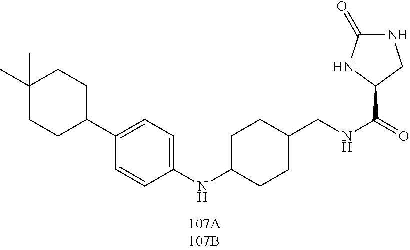 [SMILES: CC1(C)CCC(c2ccc(NC3CCC(CNC(=O)[C@@H]4CNC(=O)N4)CC3)cc2)CC1]

The title compound 107A (Rt=4.98 min; 3.7 mg) in a total yield of 15% as a white solid compound 107B (Rt=5.76 min; 5.4 mg) in a total yield of 22% as a white solid were prepared from N-(4-(aminomethyl)cyclohexyl)-4-(4,4-dimethylcyclohexyl)aniline (19 mg, 0.06 mmol) according to the procedure for 1 which were purified by preparative HPLC (Column: X Select-CSH-Prep 5 μm OBD, 19*150 mm; ACN/water (5% % TFA)=30%-45%-95%-95%-10%, 0-7 min-7.5 min-8.5 min-10.0 min). 107A: ¹H NMR (400 MHZ, DMSO-d₆) δ 7.86 (t, J=5.6 Hz, 1H), 6.91 (d, J=8.0 Hz, 2H), 6.46 (d, J=8.0 Hz, 2H), 6.30 (s, 1H), 5.10 (m, 1H), 4.05 (m, 1H), 3.52 (m, 1H), 3.18 (m, 1H), 3.07-2.91 (m, 2H), 2.21 (m, 1H), 1.70 (m, 1H), 1.64-1.24 (m, 16H), 0.94 (s, 3H), 0.91 (s, 3H). Mass (m/z): 427.2 [M+H]⁺. 107B: ¹H NMR (400 MHZ, DMSO-d₆) δ 7.88 (t, J=5.6 Hz, 1H), 6.91 (d, J=8.0 Hz, 2H), 6.51 (d, J=8.0 Hz, 2H), 6.29 (s, 1H), 5.20 (m, 1H), 4.06 (m, 1H), 3.51 (m, 1H), 3.14 (m, 1H), 3.09-2.93 (m, 2H), 2.21 (m, 1H), 1.70 (m, 1H), 1.67-1.23 (m, 16H), 0.94 (s, 3H), 0.91 (s, 3H). Mass (m/z): 427.2 [M+H]⁺.

### Compound 108

### N-(4-cyclohexylphenyl)pyrrolidin-3-amine

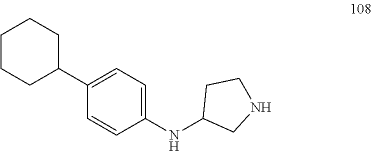 [SMILES: c1cc(C2CCCCC2)ccc1NC1CCNC1]

The title compound SIR2-1443 (20 mg) was prepared in a total yield of 83% as a white solid from tert-butyl 3-((4-cyclohexylphenyl)amino)pyrrolidine-1-carboxylate (34.1 mg, 0.1 mmol) according to the procedure for 24. ¹H NMR (400 MHZ, DMSO-d₆) δ 6.96 (d, J=8.4 Hz, 2H), 6.52 (d, J=8.4 Hz, 2H), 5.74 (s, 1H), 4.03 (m, 1H), 3.30-3.13 (m, 4H), 3.00 (m, 1H), 2.32 (m, 1H), 2.22-2.05 (m, 1H), 1.94-1.57 (m, 6H), 1.31 (m, 4H). Mass (m/z): 245.2 [M+H]⁺.

### Compound 109

### N-(4-cyclohexylphenyl)azetidin-3-amine

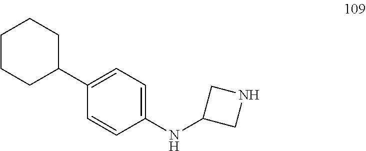 [SMILES: c1cc(C2CCCCC2)ccc1NC1CNC1]

The title compound 109 (18 mg) was prepared in a total yield of 78% as a white solid from tert-butyl 3-((4-cyclohexylphenyl)amino)azetidine-1-carboxylate (33.0 mg, 0.1 mmol) according to the procedure for 24. ¹H NMR (400 MHZ, DMSO-d₆) δ 9.04 (s, 1H), 6.96 (d, J=8.4 Hz, 2H), 6.44 (d, J=8.4 Hz, 2H), 6.21 (s, 1H), 4.28 (m, 1H), 4.20 (m, 2H), 3.77 (m, 2H), 2.32 (m, 1H), 1.80-1,63 (m, 4H), 1.36-1.17 (m, 6H). Mass (m/z): 231.3 [M+H]⁺.

### Compound 110

### N-(4-((4-cyclohexylphenyl)amino)cyclohexyl)-5-oxopyrrolidine-3-carboxamide

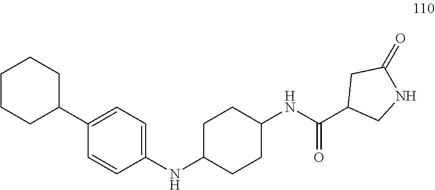 [SMILES: O=C1CC(C(=O)NC2CCC(Nc3ccc(C4CCCCC4)cc3)CC2)CN1]

The title compound 110 (12.0 mg) was prepared in a total yield of 31% as a white solid with 2:3 mixture by ¹H NMR from N1-(4-cyclohexylphenyl)cyclohexane-1,4-diamine (27.2 mg, 0.1 mmol) according to the procedure for 1. ¹H NMR (400 MHZ, DMSO-d₆) δ 7.91 (s, 0.6H), 7.84 (s, 0.4H), 7.56 (d, J=8.4 Hz, 1.2H), 6.90 (d, J=8.4 Hz, 0.8H), 6.50 (d, J=8.4 Hz, 1.2H), 6.50 (d, J=8.4 Hz, 0.8H), 5.16 (s, 0.6H), 5.10 (s, 0.4H), 3.66 (m, 1H), 3.55-3.37 (m, 2H), 3.22-3.06 (m, 2H), 2.34-2.21 (m, 3H), 1.95-1.66 (m, 10H), 1.37-1.05 (m, 8H). Mass (m/z): 384.3 [M+H]⁺.

### Compound 111

### (R)—N-(4-((4-cyclohexylphenyl)amino)cyclohexyl)-2-oxoimidazolidine-4-carboxamide

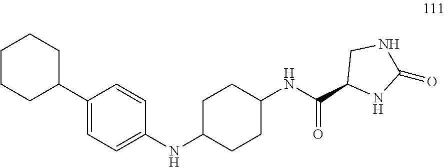 [SMILES: O=C1NC[C@H](C(=O)NC2CCC(Nc3ccc(C4CCCCC4)cc3)CC2)N1]

The title compound 111 (13.0 mg) was prepared in a total yield of 34% as a white solid with 1:1 mixture by ¹H NMR from N1-(4-cyclohexylphenyl)cyclohexane-1,4-diamine (27.2 mg, 0.1 mmol) according to the procedure for 1. ¹H NMR (400 MHZ, DMSO-d₆) δ 7.72 (s, 0.5H), 7.72 (s, 0.5H), 6.91 (d, J=8.4 Hz, 1H), 6.88 (d, J=8.4 Hz, 1H), 6.51 (m, 2H), 6.29 (s, 1H), 5.16 (s, 0.5H), 5.11 (s, 0.5H), 4.15-3.97 (m, 1H), 3.75-3.45 (m, 2H), 3.22-3.04 (m, 2H), 2.29 (m, 1H), 2.04-1.50 (m, 10H), 1.43-1.04 (m, 8H). Mass (m/z): 385.2 [M+H]⁺.

### Compound 112

### N-(3-((4-cyclohexylphenyl)amino)cyclobutyl)-5-oxopyrrolidine-3-carboxamide

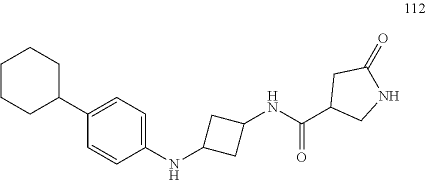 [SMILES: O=C1CC(C(=O)NC2CC(Nc3ccc(C4CCCCC4)cc3)C2)CN1]

The title compound 112 (11.6 mg) was prepared in a total yield of 33% as a white solid with 1:4 mixture by ¹H NMR from N1-(4-cyclohexylphenyl)cyclobutane-1,3-diamine (24.4 mg, 0.1 mmol) according to the procedure for 1. ¹H NMR (400 MHZ, DMSO-d₆) δ 8.37 (s, 0.8H), 8.25 (s, 0.2H), 7.58 (s, 1H), 6.92 (d, J=8.4 Hz, 1.6H), 6.89 (d, J=8.4 Hz, 0.4H), 6.42 (d, J=8.4 Hz, 0.4H), 6.38 (d, J=8.4 Hz, 1.6H), 5.76 (s, 1H), 4.29 (m, 1H), 3.92 (m, 0.2H), 3.82 (m, 0.8H), 3.41 (m, 1H), 3.26-3.04 (m, 2H), 2.35-2.17 (m, 5H), 2.11 (m, 2H), 1.78-1.64 (m, 4H), 1.37-1.11 (m, 6H). Mass (m/z): 356.2 [M+H]⁺.

### Compound 113

### (R)—N-(3-((4-cyclohexylphenyl)amino)cyclobutyl)-2-oxoimidazolidine-4-carboxamide

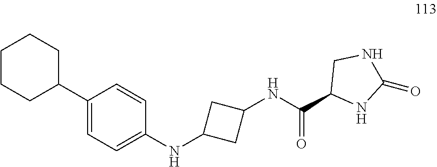 [SMILES: O=C1NC[C@H](C(=O)NC2CC(Nc3ccc(C4CCCCC4)cc3)C2)N1]

The title compound 113 (7.2 mg) was prepared in a total yield of 21% as a white solid with 1:4 mixture by ¹H NMR from N1-(4-cyclohexylphenyl)cyclobutane-1,3-diamine (24.4 mg, 0.1 mmol) according to the procedure for 1. ¹H NMR (400 MHZ, DMSO-d₆) δ 8.28 (s, 1H), 8.28 (s, 0.8H), 8.14 (s, 0.2H), 6.95 (d, J=8.4 Hz, 1.6H), 6.89 (d, J=8.4 Hz, 0.4H), 6.49 (s, 1H), 6.45 (d, J=8.4 Hz, 0.4H), 6.38 (d, J=8.4 Hz, 1.6H), 6.33 (s, 1H), 5.75 (m, 0.8H), 5.58 (m, 0.2H), 4.32 (m, 1H), 4.06 (m, 0.8H), 3.96 (m, 0.2H), 3.82 (m, 1H), 3.52 (m, 1H), 3.27-3.09 (m, 1H), 2.36-2.22 (m, 3H), 2.15-2.08 (m, 2H), 1.87-1.58 (m, 4H), 1.45-1.05 (m, 6H). Mass (m/z): 357.4 [M+H]⁺.

### Compound 114

### N-(4-((4-(tert-butyl)phenyl)amino)cyclohexyl)-5-oxopyrrolidine-3-carboxamide

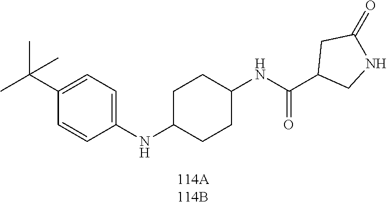 [SMILES: CC(C)(C)c1ccc(NC2CCC(NC(=O)C3CNC(=O)C3)CC2)cc1]

The title compounds 114A (Rt=6.00 min; 10.2 mg) in a total yield of 29% as a white solid and 114B (Rt=8.03 min; 10.8 mg) was prepared in a total yield of 30% as a white solid were prepared from N1-(4-(tert-butyl)phenyl)cyclohexane-1,4-diamine (49.2 mg, 0.2 mmol) according to the procedure for 1 which were purified by preparative HPLC (Column: X Select-CSH-Prep 5 μm OBD, 19*150 mm; ACN/water (5% % TFA)=20%-23%-95%-95%-0%, 0-11 min-11.5 min-12.5 min-14.0 min). 114A: ¹H NMR (400 MHZ, DMSO-d₆) δ 7.87 (d, J=7.2 Hz, 1H), 7.56 (s, 1H), 7.07 (d, J=8.8 Hz, 2H), 6.51 (d, J=8.8 Hz, 2H), 5.18 (s, 1H), 3.66 (s, 1H), 3.38 (m, 1H), 3.29 (m, 1H), 3.18 (m, 2H), 2.26 (m, 2H), 1.67-1.53 (m, 8H), 1.20 (s, 9H). Mass (m/z): 358.3 [M+H]⁺. 114B: ¹H NMR (400 MHZ, DMSO-d₆) δ 7.93 (d, J=7.6 Hz, 1H), 7.57 (s, 1H), 7.06 (d, J=8.4 Hz, 2H), 6.48 (d, J=8.4 Hz, 2H), 5.12 (s, 1H), 3.51 (m, 1H), 3.37 (m, 1H), 3.24-3.14 (m, 1H), 3.14-3.04 (m, 2H), 2.26 (m, 2H), 2.04-1.89 (m, 2H), 1.85-1.73 (m, 2H), 1.35-1.22 (m, 4H), 1.20 (s, 9H). Mass (m/z): 358.3 [M+H]⁺.

### Compound 115

### (R)—N-(4-((4-(ter-butyl)phenyl)amino)cyclohexyl)-2-oxoimidazolidine-4-carboxamide

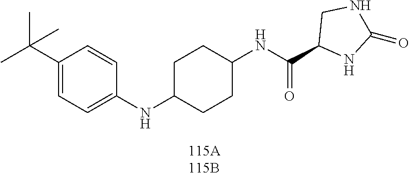 [SMILES: CC(C)(C)c1ccc(NC2CCC(NC(=O)[C@H]3CNC(=O)N3)CC2)cc1]

The title compound 115A (Rt=5.88 min; 7.0 mg) in a total yield of 20% as a white solid and 115B (Rt=7.52 min; 10.8 mg) in a total yield of 30% as a white solid were prepared from N1-(4-(tert-butyl)phenyl)cyclohexane-1,4-diamine (49.2 mg, 0.2 mmol) according to the procedure for 1, which were purified by preparative HPLC (Column: X Select-CSH-Prep 5 μm OBD, 19*150 mm; ACN/water (5% % TFA)=18%-18%-95%-95%-10%, 0-11 min-11.5 min-12.5 min-10.0 min). 115A: ¹H NMR (400 MHZ, DMSO-d₆) δ 7.76 (d, J=7.6 Hz, 1H), 7.07 (d, J=8.4 Hz, 1H), 6.54-6.45 (m, 3H), 6.29 (s, 1H), 5.16 (s, 1H), 4.03 (m, 1H), 3.51 (m, 2H), 3.19 (m, 1H), 3.15-3.03 (m, 1H), 2.04-1.89 (m, 2H), 1.84-1.75 (m, 2H), 1.43-1.26 (m, 4H), 1.20 (s, 9H). Mass (m/z): 359.2 [M+H]⁺. 115A: ¹H NMR (400 MHZ, DMSO-d₆) δ 7.61 (d, J=7.2 Hz, 1H), 7.08 (d, J=8.4 Hz, 2H), 6.59-6.51 (m, 3H), 6.30 (s, 1H), 5.22 (s, 1H), 4.08 (m, 1H), 3.69 (m, 1H), 3.51 (m, 1H), 3.32 (m, 1H), 3.20 (m, 1H), 1.75-1.47 (m, 8H), 1.20 (s, 9H). Mass (m/z): 359.2 [M+H]⁺.

### Compound 116

### 1-((4-((4-(4,4-dimethylcyclohexyl)phenyl)amino)cyclohexyl)methyl)urea

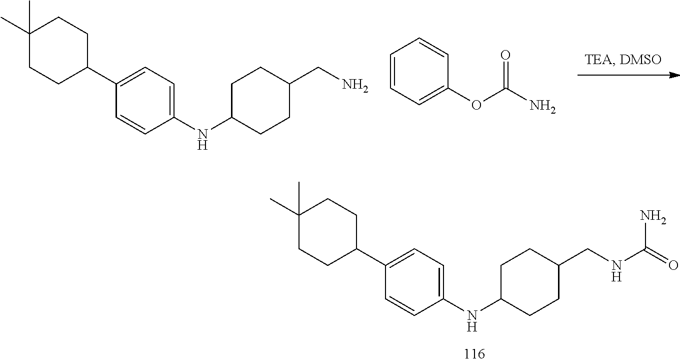

The N-(4-(aminomethyl)cyclohexyl)-4-(4,4-dimethylcyclohexyl)aniline (31.4 mg, 0.1 mmol) and triethylamine (0.028 mL, 0.2 mmol) were both dissolved in 2 mL of DMSO. Then phenylcarbamate (16.4 mg, 0.12 mmol) was added. The resulted solution was stirred overnight. Water was added to the reaction mixture and the mixture was extracted with ethyl acetate. The combined organic layers were dried over MgSO₄ and evaporated. The mixture was purified by prep-TLC to afford the desired product as a white solid (16.8 mg, 48%) with 1:4 mixture by H NMR. ¹H NMR (400 MHZ, DMSO-d₆) 6.95-6.86 (m, 2H), 6.50 (d, J=8.4 Hz, 1.2H), 6.46 (d, J=8.4 Hz, 0.8 H), 5.96 (m, 1H), 5.34 (s, 2H), 5.16 (m, 0.6H), 5.09 (m, 0.4H), 3.12-2.98 (m, 1H), 2.88 (m, 1.6H), 2.82 (m, 0.8H), 2.20 (m, 1H), 1.98 (m, 1H), 1.75-1.25 (m, 16H), 0.94 (s, 3H), 0.92 (s, 3H). Mass (m/z): 358.4 [M+H]⁺.

### Compound 117

### (R)—N-((4-((4-cyclohexylphenyl)amino)cyclohexyl)methyl)-2-oxoimidazolidine-4-carboxamide

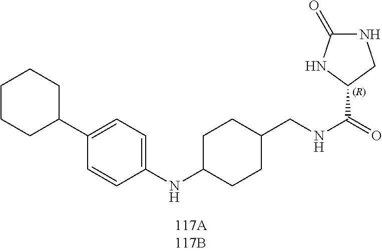

The title products 117A as a white solid (10.9 mg, 18.6%) and 117B as a white solid (10.9 mg, 13.7%) were prepared from N-(4-(aminomethyl)cyclohexyl)-4-cyclohexylaniline (57.2 mg, 0.2 mmol), (R)-2-oxoimidazolidine-4-carboxylic acid (33.8 mg, 0.26 mmol), DMF (2.0 mL), DIEA (77.4 mg, 0.6 mmol) and HATU (83.6 mg, 0.22 mmol) according to the procedure for 1. 1¹H NMR (400 MHZ, DMSO-d₆) δ 10.10 (s, 1H), 6.94-6.87 (m, 2H), 6.51 (d, J=8.6 Hz, 2H), 3.55-3.46 (m, 1H), 3.17-3.06 (m, 1H), 2.65 (s, 6H), 2.23-2.14 (m, 1H), 1.88-1.81 (m, 2H), 1.77-1.66 (m, 4H), 1.57-1.46 (m, 6H), 1.41-1.35 (m, 2H), 1.27-1.19 (m, 2H), 0.89 (d, J=8.6 Hz, 6H). Mass (m/z): 329.3 [M+H]⁺. Rt=4.778 mins (Agilent, poroshell 120, SB—C18 2.7 μm, 4.6x50 mm, ACN/Water (0.1% FA)=5%-5%-95%-95%-95%-5%, 0-0.5 min-10 min-10.5 min-12.0 min). ¹H NMR (400 MHZ, DMSO-d₆) δ 6.90 (d, J=8.0 Hz, 2H), 6.44 (d, J=8.0 Hz, 2H), 5.19 (s, 1H), 3.10 (t, J=11.2 Hz, 2H), 2.65 (t, J=4.8 Hz, 6H), 2.22-2.12 (m, 1H), 2.04-1.97 (m, 4H), 1.57-1.36 (m, 8H), 1.26-1.08 (m, 4H), 0.89 (d, J=8.4 Hz, 6H). Mass (m/z): 329.3 [M+H]⁺. Rt=4.456 mins (Agilent, poroshell 120, SB—C18 2.7 μm, 4.6x50 mm, ACN/Water (0.1% FA)=5%-5%-95%-95%-95%-5%, 0-0.5 min-10 min-10.5 min-12.0 min).

### Compound 118

### N-(4-(2-aminoethyl)cyclohexyl)-4-(4, 4-dimethylcyclohexyl)aniline

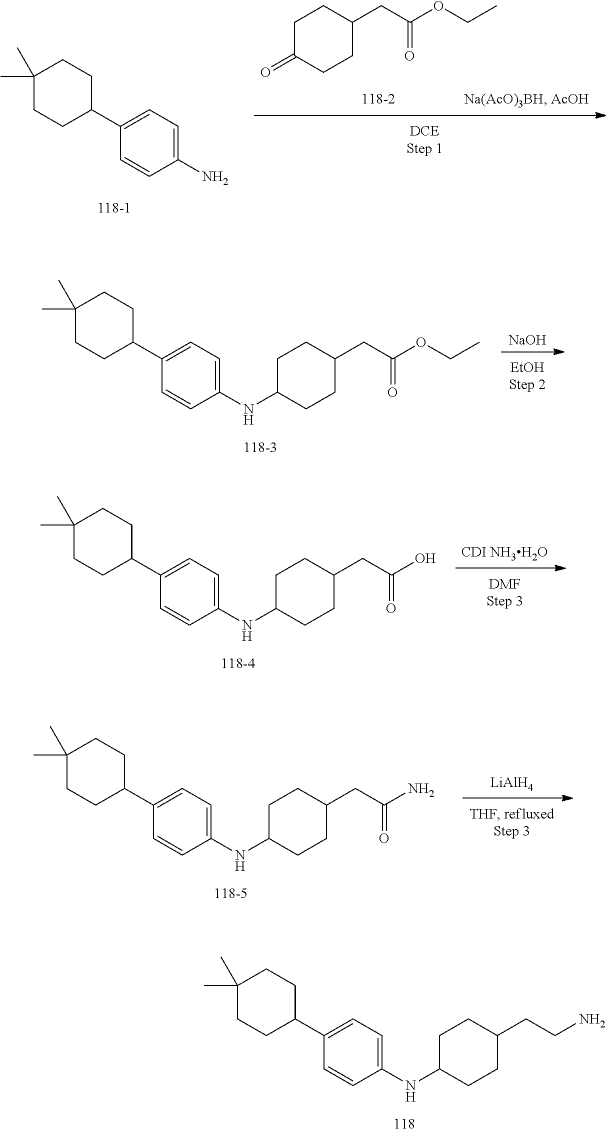

Step 1. Preparation of ethyl 2-(4-((4-(4,4-dimethylcyclohexyl)phenyl)amino)cyclohexyl) acetate (118-3)

The title compound 118-3 (231 mg) was prepared in a total yield of 62.3% as a yellow solid from 4-(4,4-dimethylcyclohexyl)aniline (203 mg, 1.0 mmol), ethyl 2-(4-((4-(4,4-dimethylcyclohexyl)phenyl)amino)cyclohexyl) acetate (368 mg, 2.0 mmol) and NaBH (OAc) 3 (424 mg, 2.0 mmol) according to the procedure for 1-1. Mass (m/z): 372.3 [M+H]⁺.

Step 2. Preparation of 2-(4-((4-(4,4-dimethylcyclohexyl)phenyl)amino)cyclohexyl) acetic acid (118-4)

To a solution of ethyl 2-(4-((4-(4,4-dimethylcyclohexyl)phenyl)amino)cyclohexyl) acetate (170 mg, 0.5 mmol) in EtOH (10 mL) was added NaOH (100 mg. 2.5 mmol). The mixture was stirred for 3 hours at 65° C., cooled to room temperature, and acidified with aqueous 2N HCl to adjust the pH to 3. The mixture was extracted with DCM (3×50 mL). The combined organic layers were washed with water, dried with Na₂SO₄, filtered, and concentrated to give a desired product yellow solid (157 mg, 100%). Mass (m/z): 342.3 [M−H]⁺.

Step 3. Preparation of 2-(4-((4-(4,4-dimethylcyclohexyl)phenyl)amino)cyclohexyl)acetamide (118-5)

To a solution of 2-(4-((4-(4,4-dimethylcyclohexyl)phenyl)amino)cyclohexyl) acetic acid (150 mg, 0.44 mmol) in DMF (2.0 mL) was added CDI (78 mg, 0.48 mmol). The mixture was stirred for 3 hours at 100° C., cooled to 0° C., NH₃·H₂O (0.1 mmol) was added. The mixture was stirred for 30 mins at rt. 5 mL of water was added, the mixture was extracted with DCM (3×5 mL). The combined organic layers were washed with water, dried with Na₂SO₄, filtered, and concentrated. The residue was purified by prep-TLC (EA/PE=1/2) to give a desired product yellow solid (60 mg, 40%). Mass (m/z): 343.3 [M+H]⁺.

Step 4. Preparation of N-(4-(2-aminoethyl)cyclohexyl)-4-(4,4-dimethylcyclohexyl)aniline (118)

A solution of 2-(4-((4-(4,4-dimethylcyclohexyl)phenyl)amino)cyclohexyl)acetamide (50 mg, 0.15 mol) in THF (10 mL) was added LiAlH₄ (24 g, 0.6 mol) at 0° C. and the mixture was refluxed for 2 h. After cooling to 0° C., water (24 uL), 10% NaOH (48 uL) and water 972 mL) were added, and the mixture was stirred for 3 min at room temperature. The solid was filtered and the filtered cake was washed with THF (10 mL*2); then the combined filtrates were dried over Na₂SO₄ and concentrated. The residue was purified by prep-TLC (MeOH/DCM=1/5) to give the desired product (16.2 mg, 34.0%) as a white solid with 7:3 mixture by ¹H NMR. 1H NMR (400 MHZ, DMSO-d₆) δ 7.84 (s, 3H), 6.89 (d, J=7.8 Hz, 2H), 6.49 (s, 2H), 3.40-3.35 (m, 0.7H), 3.09-3.00 (m, 0.3H) 2.81-2.70 (m, 2H), 2.23-2.15 (m, 1H), 1.97-1.88 (m, 1H), 1.73-1.65 (m, 1H), 1.57-1.27 (m, 16H), 0.89 (d, J=8.5 Hz, 6H). Mass (m/z): 329.3 [M+H]⁺.

### Compound 119

### 1-methyl-N4-(5,6,7,8-tetrahydronaphthalen-2-yl)cyclohexane-1,4-diamine

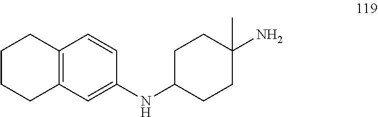 [SMILES: CC1(N)CCC(Nc2ccc3c(c2)CCCC3)CC1]

The title compound 119 (86.3 mg) was prepared in a total yield of 97.4% as a white solid from tert-butyl (1-methyl-4-((5,6,7,8-tetrahydronaphthalen-2-yl)amino)cyclohexyl)carbamate (123 mg, 0.344 mmol), TFA (1 mL), and DCM (10 mL) according to the procedure for 24. ¹H NMR (400 MHZ, DMSO-d₆) δ 8.29 (d, J=40.0 Hz, 3H), 6.77 (dd, J=14.8, 7.2 Hz, 1H), 6.55-6.26 (m, 2H), 4.98 (s, 1H), 3.25 (dt, J=8.0, 4.0 Hz, 1H), 2.58 (dd, J=16.0, 5.6 Hz, 4H), 2.01-1.84 (m, 2H), 1.80-1.71 (m, 2H), 1.70-1.64 (m, 5H), 1.52 (ddd, J=13.6, 10.0, 4.0 Hz, 2H), 1.29 (d, J=8.8 Hz, 3H). Mass (m/z): 259.3 [M+H]⁺.

### Compound 120

### N1-(4-(4,4-dimethylcyclohexyl)phenyl)cyclohexane-1,4-diamine

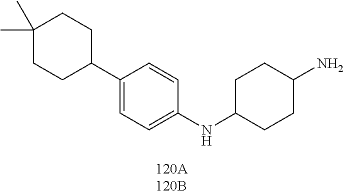 [SMILES: CC1(C)CCC(c2ccc(NC3CCC(N)CC3)cc2)CC1]

The title compound 120A (Rt=5.22 min; 18.3 mg) was prepared in a yield of 24.7% as a pale yellow powder compound 120B (Rt=5.87 min; 5.4 mg) was prepared in a yield of 7.31% as a pale yellow powder from 4-(4,4-dimethylcyclohexyl)aniline (50 mg, 0.24 mmol), tert-butyl (3-oxocyclobutyl)carbamate (79 mg, 0.37 mmol) and according to the procedure for 20, which were prepared by Prep-HPLC (Column: X Select-CSH-Prep 5 μm OBD, 19*150 mm; ACN/water (0.5% TFA)=5%-5%-95%-95%-5%, 0 min-1 min-10 min-11 min-15.0 min). 120A: ¹H NMR (400 MHZ, DMSO-d₆) δ 7.85 (s, 3H), 7.12 (s, 1H), 6.84 (s, 2H), 6.65 (s, 1H), 3.21 (s, 1H), 2.07-1.89 (m, 4H), 1.38 (qd, J=12.0, 11.3, 6.1 Hz, 2H), 1.31-1.16 (m, 11H). Mass (m/z): 301.5 [M+H]⁺. 120B: ¹H NMR (400 MHZ, DMSO-d₆) δ 7.83 (s, 2H), 7.13-6.93 (m, 1H), 6.60 (d, J=84.7 Hz, 3H), 3.47 (s, 1H), 3.14 (s, 1H), 1.88-1.56 (m, 7H), 1.24 (d, J=1.2 Hz, 9H). Mass (m/z): 301.5 [M+H]⁺.

### Compound 121

### N1-(4-(4,4-dimethylcyclohexyl)phenyl)cyclopentane-1,3-diamine

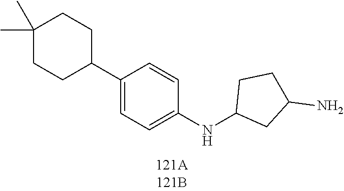 [SMILES: CC1(C)CCC(c2ccc(NC3CCC(N)C3)cc2)CC1]

The title compound 121A (Rt=5.76 min; 11.0 mg) was prepared in a yield of 7.8% as a white powder and compound 121B (Rt=5.91 mi; 6.8 mg) was prepared in a yield of 4.8% as a white powder from 4-(4,4-dimethylcyclohexyl)aniline (100 mg, 0.49 mmol), tert-butyl (3-oxocyclopentyl)carbamate (146 mg, 0.74 mmol) and according to the procedure for 20 which were prepared by Prep-HPLC (Column: X Select-CSH-Prep 5 μm OBD, 19*150 mm; ACN/water (0.5% TFA)=5%-5%-95%-95%-5%, 0 min-1 min-10 min-11 min-15.0 min). 121A: ¹H NMR (400 MHZ, DMSO-d₆) δ 7.81 (s, 3H), 7.01 (d, J=8.2 Hz, 2H), 6.63 (d, J=40.9 Hz, 2H), 3.91-3.75 (m, 1H), 3.64 (s, 1H), 2.26 (q, J=5.7 Hz, 1H), 2.16-2.05 (m, 2H), 1.99 (p, J=6.9, 6.5 Hz, 1H), 1.87 (q, J=6.0, 5.2 Hz, 2H), 1.57-1.48 (m, 5H), 1.43 (d, J=13.1 Hz, 2H), 1.30 (d, J=13.8 Hz, 2H), 0.95 (s, 3H), 0.93 (s, 3H). Mass (m/z): 287.3 [M+H]⁺. 121B: ¹H NMR (400 MHZ, DMSO-d₆) δ 7.84 (s, 3H), 7.00 (d, J=9.2 Hz, 2H), 6.56 (s, 2H), 3.71 (d, J=7.1 Hz, 1H), 3.54 (s, 1H), 2.20-2.32 (m, 1H), 1.98 (dt, J=15.4, 7.8 Hz, 3H), 1.75-1.59 (m, 2H), 1.54 (d, J=8.4 Hz, 4H), 1.43 (d, J=13.1 Hz, 3H), 1.34-1.28 (m, 2H), 0.95 (s, 3H), 0.93 (s, 3H). Mass (m/z): 287.3 [M+H]⁺.

### Compound 122

### N1-(4-cyclohexylphenyl)cyclohexane-1,4-diamine

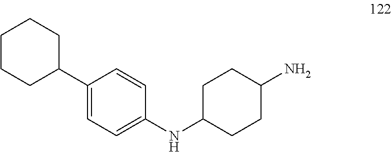 [SMILES: NC1CCC(Nc2ccc(C3CCCCC3)cc2)CC1]

The title compound 122 (2.2 mg) was prepared in a yield of 2.8% as a rosy brown solid from 4-cyclohexylaniline (50 mg, 0.27 mmol), tert-butyl (3-oxocyclobutyl)carbamate (91 mg, 0.43 mmol) and according to the procedure for 20. ¹H NMR (400 MHZ, Methanol-d₄) δ 7.20 (d, J=8.3 Hz, 2H), 6.99 (d, J=8.1 Hz, 2H), 3.58 (s, 1H), 2.48 (s, 1H), 2.02 (q, J=6.6 Hz, 1H), 1.95-1.77 (m, 12H), 1.74 (d, J=13.0 Hz, 1H), 1.51-1.36 (m, 4H), 1.31 (s, 1H). Mass (m/z): 273.3 [M+H]⁺.

### Compound 123

### N1-(4-(tert-butyl)phenyl)cyclobutane-1,3-diamine

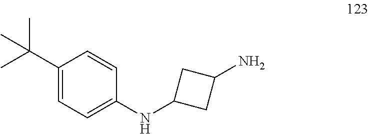 [SMILES: CC(C)(C)c1ccc(NC2CC(N)C2)cc1]

The title compound 123 (48.7 mg) was prepared in a yield of 33.2% as a white powder with 4:1 mixture by ¹H NMR from 4-(tert-butyl)aniline (100 mg, 0.67 mmol), tert-butyl (3-oxocyclobutyl)carbamate (186 mg, 1.1 mmol) and according to the procedure for 20. ¹H NMR (400 MHZ, DMSO-d₆) δ 8.43 (s, 3H), 7.12-7.08 (m, 2H), 6.43-6.37 (m, 2H), 5.83 (d, J=6.3 Hz, 1H), 4.07 (h, J=6.6 Hz, 1H), 3.72 (td, J=8.2, 4.2 Hz, 1H), 2.45 (td, J=7.7, 3.8 Hz, 2H), 2.15 (ddd, J=13.2, 8.1, 5.0 Hz, 2H), 1.20 (d, J=0.9 Hz, 9H). Mass (m/z): 219.2 [M+H]⁺.

### Compound 124

### N1-(4-(tert-butyl)phenyl)cyclopentane-1,3-diamine

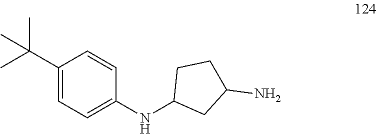 [SMILES: CC(C)(C)c1ccc(NC2CCC(N)C2)cc1]

The title compound 124 (40.0 mg) was prepared in a yield of 25.6% as a white powder with 1:0.3 mixture by ¹H NMR from 4-(tert-butyl)aniline (100 mg, 0.67 mmol), tert-butyl (3-oxocyclopentyl)carbamate (200 mg, 1.01 mmol) and according to the procedure for 20. H NMR (400 MHZ, DMSO-d₆) δ 8.15 (s, 4H), 7.11-7.03 (m, 3H), 6.49 (dd, J=8.7, 6.8 Hz, 3H), 5.65 (s, 0.3H), 5.45 (d, J=7.0 Hz, 1H), 3.88 (h, J=6.2 Hz, 1H), 3.71 (s, 0.3H), 3.62-3.54 (m, 1H), 3.48 (p, J=7.1 Hz, 0.3H), 2.38 (dt, J=14.2, 7.3 Hz, 0.3H), 2.09 (tdd, J=13.0, 10.0, 6.1 Hz, 2H), 2.01-1.87 (m, 2H), 1.86-1.70 (m, 2H), 1.68-1.54 (m, 1H), 1.52-1.41 (m, 2H), 1.26-1.21 (m, 2H), 1.20 (s, 12H). Mass (m/z): 233.3 [M+H]⁺.

### Compound 125

### N1-(4-ethylphenyl)cyclohexane-1,4-diamine

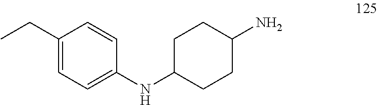 [SMILES: CCc1ccc(NC2CCC(N)CC2)cc1]

The title compound 125 (36.0 mg) was prepared in a yield of 19.9% as a white powder with 2:1 mixture by ¹H NMR from 4-ethylaniline (100 mg, 0.82 mmol), tert-butyl (4-oxocyclohexyl)carbamate (263 mg, 1.24 mmol) and according to the procedure for 20. H NMR (400 MHZ, DMSO-d₆) δ 8.01 (s, 3H), 6.90 (dd, J=8.4, 6.3 Hz, 2H), 6.56-6.45 (m, 2H), 5.21-5.11 (m, 1H), 3.09 (s, 1H), 2.97 (s, 0.46H), 2.43 (qd, J=7.6, 2.6 Hz, 2H), 1.99 (t, J=13.8 Hz, 2H), 1.84-1.66 (m, 3H), 1.61 (d, J=11.3 Hz, 1H), 1.44 (q, J=12.8, 12.2 Hz, 1H), 1.22-1.12 (m, 1H), 1.12-1.07 (m, 3H). Mass (m/z): 219.4 [M+H]⁺.

### Compound 126

### N1-(2-(tert-butyl)phenyl)cyclohexane-1,4-diamine (126)

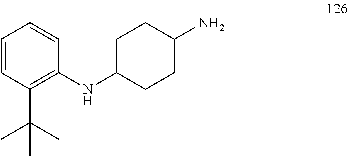 [SMILES: CC(C)(C)c1ccccc1NC1CCC(N)CC1]

The title compound 126 (54.8 mg) was prepared in a yield of 33.1% as a white powder with 8:1 mixture by ¹H NMR from 2-(tert-butyl)aniline (93 mg, 0.63 mmol), tert-butyl (4-oxocyclohexyl)carbamate (200 mg, 0.94 mmol) and according to the procedure for 20. ¹H NMR (400 MHZ, DMSO-d₆) δ 8.20 (s, 3H), 7.11 (dd, J=7.8, 1.6 Hz, 1H), 7.02 (ddd, J=8.4, 7.2, 1.5 Hz, 1H), 6.71-6.65 (m, 1H), 6.55 (tt, J=7.6, 1.7 Hz, 1H), 3.82 (d, J=8.0 Hz, 1H), 3.33-3.21 (m, 1H), 3.05-2.92 (m, 1H), 2.13-1.96 (m, 4H), 1.82 (d, J=9.2 Hz, 1H), 1.50 (qd, J=12.7, 3.2 Hz, 2H), 1.39 (s, 1H), 1.34 (s, 8H). Mass (m/z): 247.3 [M+H]⁺.

### Compound 127

### N1-(4-isopropylphenyl)cyclohexane-1,4-diamine

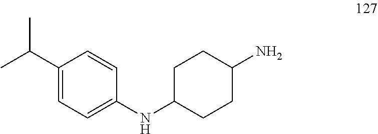 [SMILES: CC(C)c1ccc(NC2CCC(N)CC2)cc1]

The title compound 127 (56.4 mg) was prepared in a yield of 39.0% as a white powder with 2:1 mixture by ¹H NMR from 4-isopropylaniline (85 mg, 0.63 mmol), tert-butyl (4-oxocyclohexyl)carbamate (200 mg, 0.94 mmol) and according to the procedure for 20. ¹H NMR (400 MHZ, DMSO-d₆) δ 8.21 (d, J=32.0 Hz, 3H), 6.97-6.89 (m, 2H), 6.52 (dd, J=13.3, 8.3 Hz, 2H), 5.19 (s, 1H), 3.08 (s, 1H), 2.95 (s, 1H), 2.72 (ddd, J=13.8, 6.9, 2.1 Hz, 1H), 2.05-1.93 (m, 2H), 1.75 (tt, J=10.4, 5.2 Hz, 3H), 1.66-1.55 (m, 1H), 1.53-1.39 (m, 1H), 1.18 (t, J=10.9 Hz, 1H), 1.14 (d, J=1.3 Hz, 3H), 1.12 (d, J=1.3 Hz, 3H). Mass (m/z): 233.3 [M+H]⁺.

### Compound 128

### N1-(p-tolyl)cyclohexane-1,4-diamine

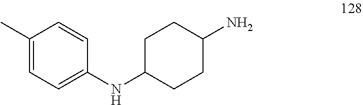

The title compound 128 (27.7 mg) was prepared in a yield of 23.0% as a white powder with 3:2 mixture by ¹H NMR from p-toluidine (63 mg, 0.59 mmol), tert-butyl (4-oxocyclohexyl)carbamate (188 mg, 0.89 mmol) and according to the procedure for 20. ¹H NMR (400 MHZ, DMSO-d₆) δ 8.31-8.21 (m, 2H), 8.17 (s, 1H), 6.87 (dd, J=8.1, 4.8 Hz, 2H), 6.56-6.43 (m, 2H), 5.15 (s, 1H), 3.08 (s, 1H), 2.95 (s, 1H), 2.13 (d, J=2.1 Hz, 3H), 2.00 (dd, J=13.0, 4.1 Hz, 3H), 1.75 (tt, J=12.3, 10.0, 4.3 Hz, 2H), 1.60 (d, J=12.8 Hz, 1H), 1.54-1.39 (m, 1H), 1.15 (q, J=12.0 Hz, 1H). Mass (m/z): 205.2 [M+H]⁺.

### Compound 129

### (R)—N-(3-((4-(4,4-dimethylcyclohexyl)phenyl)amino)cyclobutyl)-2-oxoimidazolidine-4-carboxa mide

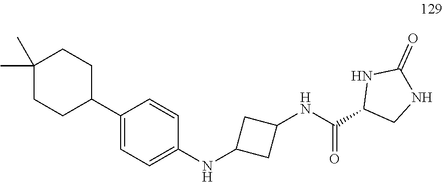 [SMILES: CC1(C)CCC(c2ccc(NC3CC(NC(=O)[C@H]4CNC(=O)N4)C3)cc2)CC1]

The title compound 129 (7.1 mg) was prepared in a yield of 16.77% as a white powder with 1:0.3 mixture by ¹H NMR from N1-(4-(4,4-dimethylcyclohexyl)phenyl)cyclobutane-1,3-diamine (30 mg, 0.11 mmol), (R)-2-oxoimidazolidine-4-carboxylic acid (19 mg, 0.14 mmol) and according to the procedure for 10. ¹H NMR (400 MHZ, DMSO-d₆) δ 8.32 (d, J=7.1 Hz, 1H), 6.99-6.90 (m, 2H), 6.51 (s, 1H), 6.43-6.37 (m, 2H), 6.30 (s, 1H), 5.77 (d, J=5.9 Hz, 1H), 4.33 (q, J=7.0 Hz, 1H), 4.05 (ddd, J=9.7, 6.1, 1.6 Hz, 1H), 3.84 (s, 1H), 2.25 (ddd, J=20.3, 11.4, 5.3 Hz, 2H), 2.12 (td, J=8.2, 4.1 Hz, 2H), 2.00 (p, J=6.9, 6.5 Hz, 2H), 1.59-1.50 (m, 4H), 1.50-1.38 (m, 4H), 1.13 (dd, J=6.9, 1.4 Hz, 1H), 0.95 (s, 3H), 0.93 (s, 3H). Mass (m/z): 385.3 [M+H]⁺.

### Compound 130

### N-((1H-imidazol-5-yl)methyl)-4-(tert-butyl)aniline

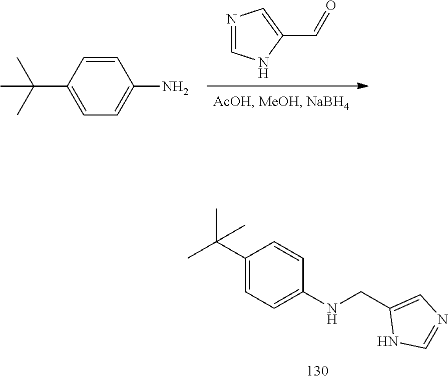 [SMILES: CC(C)(C)c1ccc(N)cc1 ; O=Cc1cnc[nH]1 ; CC(C)(C)c1ccc(NCc2cnc[nH]2)cc1]

To a solution of 4-(tert-butyl)aniline (150 mg, 1.0 mmol) in MeOH (10 mL) and a drop of AcOH was added 1H-imidazole-5-carbaldehyde (201.9 mg, 1.0 mmol) and the mixture was stirred at 50° C. for 1 h. Then the mixture was added NaBH₄ (76 mg, 2 mmol) after cooling to 25° C. Then the mixture was stirred at 25° C. for 16 hrs. LCMS showed the reaction was completed. The reaction was quenched with water (10 mL), extracted with EA (10 mL*3). The combined organic layers were washed with brine (20 mL), dried over sodium sulfate, filtered and concentrated. The residue was purified by combi-flash with EA/PE (1:3) to afford to give 130 (0.12 g, 52.0% yield) as a yellow solid. ¹H NMR (400 MHZ, DMSO-d₆) δ 7.52 (d, J=1.2 Hz, 1H), 7.05-7.02 (m, 2H), 6.87 (d, J=0.7 Hz, 1H), 6.54-6.51 (m, 2H), 5.54 (d, J=1.2 Hz, 1H), 4.05 (s, 2H), 1.16 (s, 9H). MS (m/z) 230.2 [M+H]⁺.

### Compound 131

### N-(4-(4,4-dimethylcyclohexyl)phenyl)piperidin-4-amine

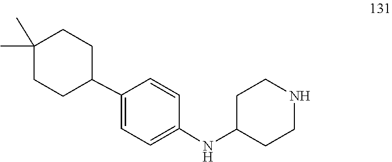 [SMILES: CC1(C)CCC(c2ccc(NC3CCNCC3)cc2)CC1]

The title compound 131 (14.7 mg) was prepared in a yield of 15.4% as a white powder from 4-(4,4-dimethylcyclohexyl)aniline (50 mg, 0.25 mmol), tert-butyl 4-oxopiperidine-1-carboxylate (74 mg, 0.37 mmol) and according to the procedure for 20. ¹H NMR (400 MHZ, DMSO-d₆) δ 8.57 (s, 1H), 8.39 (s, 1H), 7.00 (d, J=8.1 Hz, 2H), 6.62 (d, J=8.0 Hz, 2H), 3.49 (p, J=5.8 Hz, 1H), 3.30 (s, 2H), 2.97 (q, J=11.2 Hz, 2H), 2.25 (tt, J=10.5, 5.2 Hz, 1H), 2.07-1.94 (m, 2H), 1.53 (dt, J=10.8, 3.4 Hz, 5H), 1.48-1.38 (m, 3H), 1.31-1.23 (m, 2H), 0.93 (s, 3H), 0.91 (s, 3H). Mass (m/z): 287.5 [M+H]⁺.

### Compound 132

### N1-(4-cyclohexylphenyl)cyclobutane-1,3-diamine

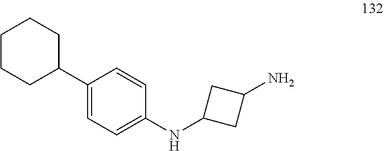 [SMILES: NC1CC(Nc2ccc(C3CCCCC3)cc2)C1]

The title compound 132 (20.3 mg) was prepared in a yield of 14.5% as a white powder with 9:1 mixture by ¹H NMR from 4-cyclohexylaniline (100 mg, 0.57 mmol), tert-butyl (3-oxocyclobutyl)carbamate (158 mg, 0.86 mmol) and according to the procedure for 20. H NMR (400 MHZ, DMSO-d₆) δ 8.38 (s, 2H), 6.97-6.87 (m, 2H), 6.41-6.36 (m, 2H), 5.81 (d, J=6.2 Hz, 1H), 4.05 (q, J=5.9 Hz, 1H), 3.72 (ddd, J=13.3, 8.2, 5.2 Hz, 1H), 2.44 (tt, J=7.7, 5.1 Hz, 2H), 2.30 (s, 1H), 2.14 (ddd, J=13.0, 8.0, 4.9 Hz, 2H), 1.80-1.63 (m, 5H), 1.29 (d, J=11.7 Hz, 4H), 1.26-1.10 (m, 1H). Mass (m/z): 245.3 [M+H]⁺.

### Compound 133

### N1-(4-cyclohexylphenyl)cyclohexane-1,4-diamine

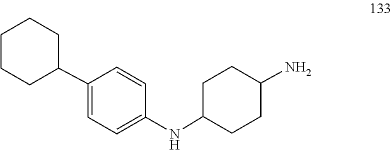 [SMILES: NC1CCC(Nc2ccc(C3CCCCC3)cc2)CC1]

The title compound 133 (5.0 mg) was prepared in a yield of 6.1% as a white powder from 4-cyclohexylaniline (50 mg, 0.27 mmol), tert-butyl (3-oxocyclobutyl)carbamate (91 mg, 0.43 mmol) and according to the procedure for 20. ¹H NMR (400 MHZ, DMSO-d₆) δ 7.78 (s, 3H), 7.02 (s, 2H), 6.67 (s, 1H), 3.15 (s, 1H), 2.99 (s, 1H), 2.35 (s, 1H), 2.07-1.86 (m, 6H), 1.83-1.62 (m, 6H), 1.39-1.28 (m, 6H). Mass (m/z): 273.3 [M+H]⁺.

### Compound 134

### N1-(4-fluorophenyl)cyclohexane-1,4-diamine

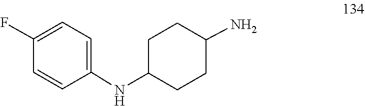 [SMILES: NC1CCC(Nc2ccc(F)cc2)CC1]

The title compound 134 (56.2 mg) was prepared in a total yield of 40.1% as a purple solid with 1:2 mixture by ¹H NMR from tert-butyl (4-((4-fluorophenyl)amino)cyclohexyl)carbamate (207 mg, 0.672 mmol), TFA (1 mL), and DCM (10 mL) according to the procedure for 24. ¹H NMR (400 MHZ, DMSO-d₆) δ 7.93 (s, 3H), 6.98 (t, J=8.9 Hz, 2H), 6.75 (dd, J=8.8, 4.6 Hz, 2H), 3.44-3.36 (m, 1H), 3.13 (dp, J=11.5, 6.1, 5.5 Hz, 1H), 1.78-1.55 (m, 8H). Mass (m/z): 209.3 [M+H]⁺.

### Compound 135

### N1-(4-propylphenyl)cyclohexane-1,4-diamine

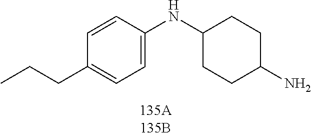 [SMILES: CCCc1ccc(NC2CCC(N)CC2)cc1]

The title compound 135A (35.5 mg) as a white solid and 135B (42.8 mg) as a white solid were prepared from tert-butyl (4-((4-cyclopentylphenyl)amino)cyclohexyl)carbamate (170 mg, 5.12 mmol) and HCl in 1,4-dioxane (10 mL, 4 N) according to the literature for 24. 135A: 1H NMR (400 MHZ, CD₃OD) δ 7.41-7.10 (m, 4H), 3.47 (brs, 1H), 3.14 (brs, 1H), 2.65-2.55 (m, 2H), 2.13 (d, J=9.8 Hz, 4H), 1.70-1.45 (m, 6H), 0.94 (t, J=7.2 Hz, 3H). MS (m/z): 233 [M+H]⁺. HPLC: Rt=3.384 min (Column: XBRIDGE 3.5 μm 2.1*50 mm, Mobile phase: H₂O (0.05% TFA)-ACN (0.05% TFA), ACN from 0 to 60% over 7 minutes, Flow rate: 0.8 mL/min). 135B: ¹H NMR (400 MHZ, CD₃OD) δ 7.44-7.30 (m, 4H), 3.66 (br, 1H), 3.46 (br, 1H), 2.64 (dd, J=15.5, 8.2 Hz, 3H), 2.14-1.81 (m, 6H), 1.76-1.55 (m, 3H), 0.94 (td, J=7.3, 5.2 Hz, 4H). MS (m/z): 233 [M+H]⁺. HPLC: Rt=3.580 min (Column: XBRIDGE 3.5 μm 2.1*50 mm, Mobile phase: H₂O (0.05% TFA)-ACN (0.05% TFA), ACN from 0 to 60% over 7 minutes, Flow rate: 0.8 mL/min).

### Compound 136

### N1-(4-pentylphenyl)cyclohexane-1,4-diamine

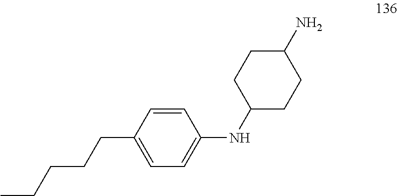 [SMILES: CCCCCc1ccc(NC2CCC(N)CC2)cc1]

The title compound 136 (55 mg, 95.4%) was prepared as a white solid from tert-butyl (4-((4-pentylphenyl)amino)cyclohexyl)carbamate (79 mg, 0.22 mmol) and HCl in 1,4-dioxane (10 mL, 4 N) according to the procedure for 24. ¹H NMR (400 MHz, CD₃OD) δ 7.44-7.12 (m, 4H), 3.69-3.37 (m, 2H), 3.20-3.08 (m, 1H), 2.70-2.57 (m, 2H), 2.15 (br, 2H), 1.91 (brs, 4H), 1.57 (br, 4H), 1.41-1.24 (br, 4H), 0.90 (t, J=6.9 Hz, 3H). MS (m/z) 261.3 [M+H]⁺.

### Compound 137

### N1-(4-butylphenyl)cyclohexane-1,4-diamine

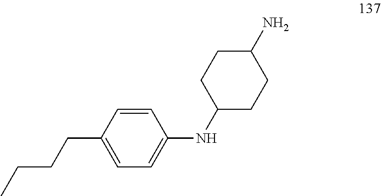 [SMILES: CCCCc1ccc(NC2CCC(N)CC2)cc1]

The title compound 137 (78 mg,79.2%) was prepared as a white solid from tert-butyl (4-((4-butylphenyl)amino)cyclohexyl)carbamate (90 mg, 0.26 mmol) and HCl in 1,4-Dioxane (10 mL, 4 N) according to the procedure for 37. ¹H NMR (400 MHZ, CDCl₃) δ 6.98 (d, J=8.4 Hz, 2H), 6.55 (d, J=8.4 Hz, 2H), 3.26 (br, 1H), 3.08 (br, 1H), 2.55-2.40 (m, 2H), 2.30-2.08 (m, 4H), 1.65-1.50 (m, 4H), 1.35-1.30 (m, 2H), 1.29-1.24 (m, 2H), 1.24-1.08 (m, 2H), 0.91 (t, J=7.2 Hz, 3H). MS (m/z): 247 [M+H]⁺.

### Compound 138

### N1-(4-(2,6-dimethyltetrahydro-2H-pyran-4-yl)phenyl)cyclohexane-1,4-diamine

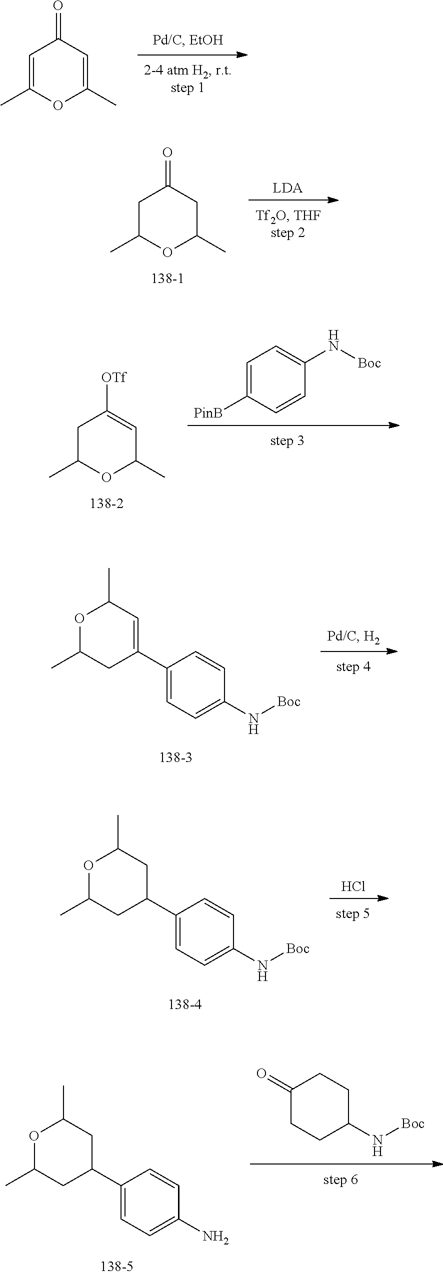

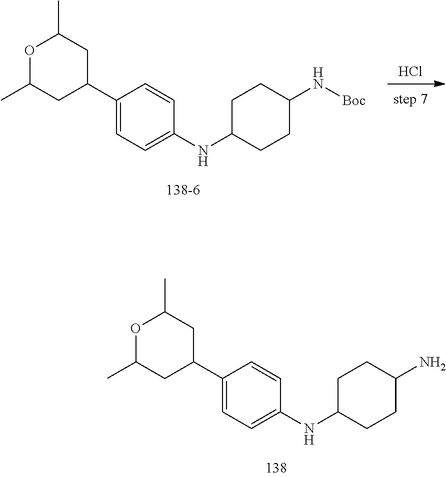 [SMILES: CC1CC(c2ccc(NC3CCC(NC(=O)OC(C)(C)C)CC3)cc2)CC(C)O1 ; Cl ; CC1CC(c2ccc(NC3CCC(N)CC3)cc2)CC(C)O1]

Step 1. Preparation of 2,6-dimethyltetrahydro-4H-pyran-4-one (138-1)

To a solution of 2,6-dimethyl-4H-pyran-4-one (5 g, 40.3 mmol) in etOH (200 mL) was added Palladium (1 g). Then the mixture was stirred under H₂ in 3 bar at 25° C. for 8 h. After filtrating the Palladium, the organic phase was removed under vacuum and the residue was purified by flash chromatography to afford the desired product (1.4 g, 63.7%) as colorless oil. ¹H NMR (400 MHZ, DMSO-d₆) δ 3.76-3.64 (m, 2H), 2.22-2.18 (m, 4H), 1.20 (d, J=8 Hz, 6H).

Step 2. Preparation of 2,6-dimethyl-3,6-dihydro-2H-pyran-4-yl trifluoromethanesulfonate (138-2)

To a solution of 2,6-dimethyltetrahydro-4H-pyran-4-one (0.7 g, 5.47 mmol) in THF (20 mL) was added LDA (10 mL, 6.56 mmol) and the mixture was stirred for 30 min at −78° C. under N₂. Then trifluoroacetic anhydride (5.2 g 6.56 mmol) was dripped into the mixture and stirred at 25° C. overnight. The reaction was quenched with water (30 mL), and extracted by EA (10 mL) for 3 times. The combined organic phase was washed 3 times by NaOH aq (10 mL) and dried over sodium sulfate, removed under vacuum to obtain the crude product (0.6 g, 42.1%) as yellow oil. ¹H NMR (400 MHZ, DMSO-d₆) δ 6.81-6.76 (m, 1H), 3.81-3.71 (m, 2H), 3.34 (s, 2H), 2.50 (s, 6H).

Step of tert-butyl 3. Preparation (4-(2,6-dimethyl-3,6-dihydro-2H-pyran-4-yl)phenyl)carbamate (138-2)

To a solution of 2,6-dimethyl-3,6-dihydro-2H-pyran-4-yl trifluoromethanesulfonate (0.6 g, 2.3 mmol) in dioxane: H₂O=4:1 (20 mL) was added 4-(N-Boc-amino)phenylboronic acid pinacol ester (733 mg, 2.3 mmol), potassium carbonate (952 mg, 6.9 mmol) and tetrakis(triphenylphosphine) palladium (265 mg, 0.23 mmol). Then the mixture was stirred at 90° C. for 12 h. The reaction was quenched with water (30 mL), and extracted by EA (10 mL) for 3 times. The combined organic phase was dried over sodium sulfate, concentrated under vacuum and the residue was purified by flash chromatography to afford the desired product (0.35 g, 50.2%) as yellow oil. Mass (m/z): 248.2 [M+H]⁺.

Step 4. Preparation of tert-butyl (4-(2,6-dimethyltetrahydro-2H-pyran-4-yl)phenyl)carbamate (138-3)

To a solution of tert-butyl (4-((2R,6S)-2,6-dimethyl-3,6-dihydro-2H-pyran-4-yl)phenyl)carbamate (0.35 g, 1.16 mmol) in MeOH (10 mL) was added Palladium (80 mg). Then the mixture was stirred at 25° C. for 3 h under N₂. After filtrating the Palladium, the organic phase was removed under vacuum and the residue was purified by flash chromatography to afford the desired product as yellow oil (0.33 g, 98.3%). Mass (m/z): 250.2 [M+H]⁺.

Step 5. Preparation of 4-(2,6-dimethyltetrahydro-2H-pyran-4-yl)aniline (138-4)

To a solution of tert-butyl (4-(2,6-dimethyltetrahydro-2H-pyran-4-yl)phenyl)carbamate (0.33 g, 1.08 mmol) in THF (5 mL) was added HCl in dioxane (5 mL) and the mixture was stirred for 2 h. The reaction was quenched with NaHCO₃ (10 mL), extracted by EA (10 mL) for 3 times and dried over sodium sulfate. After filtration, the organic phase was removed under vacuum and the residue was purified by flash chromatography to afford the desired product (0.16 g, 72.3%) s white solid. ¹H NMR (400 MHZ, CDCl₃) δ 7.00 (d, J-8 Hz, 2H), 6.65 (d, J-8 Hz, 2H), 3.58 (m, 3H), 2.69 (m, 1H), 1.78-1.74 (m, 2H), 1.35-1.26 (m, 2H), 1.24 (d, J=4 Hz, 6H). Mass m/z): 220.2 [M+H]

Step 6. Preparation of tert-butyl (4-((4-(2,6-dimethyltetrahydro-2H-pyran-4-yl)phenyl)amino) cyclohexyl)carbamate (138-5)

To a solution of 4-(2,6-dimethyltetrahydro-2H-pyran-4-yl)aniline (60 mg, 0.29 mmol) in MeOH (5 mL was added tert-butyl (4-oxocyclohexyl)carbamate (62.3 mg, 0.29 mmol) and acetic acid (1.74 mg, 0.029 mmol). Then the mixture was stirred at 60° C. for 3 h. After the reaction was cooled to R.T., sodium cyanoborohydride (54.8 mg, 0.87 mmol) was added. The reaction was stirred for 3 h at R.T. The reaction was quenched with water (10 mL), extracted by EA (10 mL) for 3 times and dried over sodium sulfate. After filtration, the organic phase was removed under vacuum and the residue was purified by flash chromatography to afford the desired product (70 mg, 58.6%) as white solid. Mass (m/z): 402.8 [M+H]⁺.

Step 7. Preparation of N1-(4-(2,6-dimethyltetrahydro-2H-pyran-4-yl)phenyl) cyclohexane-1,4-diamine (138)

To a solution of 4-(2,6-dimethyltetrahydro-2H-pyran-4-yl)aniline (70 mg, 0.17 mmol) in THF (5 mL) was added HCl in dioxane (4N; 5 mL) and the mixture was stirred for 2 h. The reaction was quenched with NaHCO; (10 mL), extracted by EA (10 mL) for 3 times and dried over sodium sulfate. After filtration, the organic phase was removed under vacuum and the residue was purified by prep-HPLC (column-Xbridge-C18 150×21.2 mm, 5 μm; Mobile phase: ACN-H₂O (0.1% FA), 40%-60%) to afford the desired product (32 mg, 62.3%) as a white solid. H NMR (400 MHZ, CDCl₃) δ 7.00 (d, J=8.4 Hz, 2H), 6.57 (d, J=8.4 Hz, 2H), 3.57 (m, 3H), 3.19 (s, 1H), 2.66 (m, 1H), 1.95-1.58 (m, 10H), 1.37-1.13 (m, 9H). Mass (m/z): 303.3 [M+H]⁺.

### Compound 139

### N1-(4-cyclopentylphenyl)cyclohexane-1,4-diamine

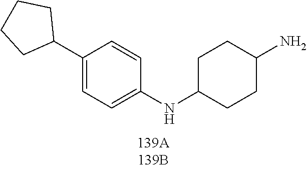 [SMILES: NC1CCC(Nc2ccc(C3CCCC3)cc2)CC1]

The title compounds 139A (46.5 mg) as a white solid and 139B (32.9 mg) as a white solid were prepared from tert-butyl (4-((4-cyclopentylphenyl)amino)cyclohexyl)carbamate (120 mg, 0.33 mmol) and HCl in 1,4-dioxane (10 mL, 4 N) according to the procedure for 24. 139A: 1H NMR (400 MHZ, CD₃OD) δ 7.37 (br, 2H), 7.20 (br, 2H), 3.58 (br, 1H), 3.39 (br, 1H), 3.19-2.97 (m, 1H), 2.13 (br, 2H), 2.00-1.34 (m, 14H). MS (m/z) 259 [M+H]⁺. HPLC: Rt=3.787 min (Column: XBRIDGE 3.5 μm 2.1*50 mm, Mobile phase: H₂O (0.05% TFA)-ACN (0.05% TFA), ACN from 0 to 60% over 7 minutes, Flow rate: 0.8 mL/min). 139B: ¹H NMR (400 MHZ, CD₃OD) δ 7.37 (br, 2H), 7.20 (br, 2H), 3.58 (br, 1H), 3.39 (br, 1H), 3.19-2.97 (m, 1H), 2.13 (br, 2H), 2.00-1.34 (m, 14H). MS (m/z) 259 [M+H]⁺. HPLC: Rt=3.991 min (Column: XBRIDGE 3.5 μm 2.1*50 mm, Mobile phase: H₂O (0.05% TFA)-ACN (0.05% TFA), ACN from 0 to 60% over 7 minutes, Flow rate: 0.8 mL/min).

### Compound 140

### N-(4-(1-aminoethyl)cyclohexyl)-4-(4, 4-dimethylcyclohexyl)aniline

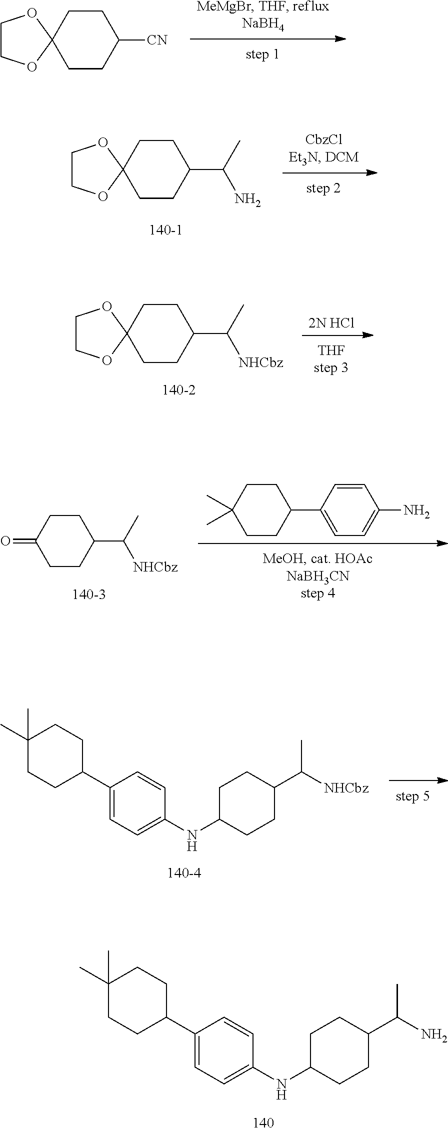

Step 1. Preparation of 1-(1,4-dioxaspiro[4.5]decan-8-yl) ethan-1-amine (140-1)

To a solution of 1,4-dioxaspiro[4.5]decane-8-carbonitrile (1 g, 6 mmol) in THF (10 mL) was added MeMgBr (2 mL, 6 mmol) at 0° C. under N₂. Then the mixture was stirred at 70° C. for 2 hrs. Then the mixture was added NaBH₄ (0.68 g, 18 mmol) after cooling to 25° C. Then the mixture was stirred at 25° C. for 2 hrs. LCMS showed the reaction was completed. The reaction was quenched with water (20 mL), extracted with EA (20 mL*3). The combined organic layers were washed with brine (40 mL), dried over sodium sulfate, filtered and concentrated. The residue was purified by combi-flash with EA/PE (1:0) to give compound 140-1 (0.2 g, 18.2% yield) as a yellow solid. MS (m/z) 186.2 [M+H]⁺.

Step 2. Preparation of benzyl (1-(1,4-dioxaspiro[4.5]decan-8-yl)ethyl)carbamate (140-2)

To a solution of compound 140-1 (0.2 g, 1.1 mmol) in DCM (10 mL) was added TEA (218.5 mg, 2.2 mmol) and CbzCl (202.6 mg, 1.2 mmol). Then the mixture was stirred at 25° C. for 2 hrs. LCMS showed the reaction was completed. The mixture was added into H₂O, extracted with EA (20 mL*3). The combined organic layers were washed with brine (40 mL), dried over sodium sulfate, filtered and concentrated. The residue was purified by combi-flash with EA/PE (1:3) to give compound 140-2 (0.2 g, 58% yield) as yellow oil. MS (m/z) 342.2 [M+Na]⁺.

Step 3. Preparation of benzyl (1-(4-oxocyclohexyl)ethyl)carbamate (140-3)

To a solution of 140-2 (0.2 g, 0.62 mmol) in THF (2 mL) was added 2N HCl (2 mL) at 25° C. Then the mixture was stirred at 25° C. for 10 h. LCMS showed the reaction was completed. The reaction was concentrated. The residue was purified by combi-flash with EA/PE (1:2) to afford compound 4 (0.105 g, 61% yield) as yellow oil. MS (m/z) 275.8 [M+H]⁺.

Step 4. Preparation of benzyl (1-(4-((4-(4,4-dimethylcyclohexyl)phenyl)amino)cyclohexyl)ethyl)carbamate (140-4)

To a solution of 140-3 (105 mg, 0.36 mmol) in MeOH (5 mL) and a drop of AcOH was added 4-(4,4-dimethylcyclohexyl)aniline (74 mg, 0.36 mmol) and the mixture was stirred at 50° C. for 1 h. Then the mixture was added NaBH₃CN (68.5 mg, 1.1 mmol) after cooling to 25° C. Then the mixture was stirred at 25° C. for 2 hrs. LCMS showed the reaction was completed The reaction was quenched with water (10 mL), extracted with FA (10 mL*3). The combined organic layers were washed with brine (20 mL), dried over sodium sulfate, filtered and concentrated. The residue was purified by combi-flash with EA/PE (1:3) to give compound 5 (0.1 g, 59.5% yield) as a yellow solid. MS (m/z) 462.8 [M+H]⁺.

Step 5. Preparation of N-(4-(1-aminoethyl)cyclohexyl)-4-(4,4-dimethylcyclohexyl)aniline (140)

To a solution of compound 140-4 (0.1 g, 0.21 mmol) in DCM (5 mL) was added Et₃SiH (75.4 mg, 0.65 mmol), TEA (44 mg, 0.43 mmol) and Pd (OAc) 2 (5 mg, 0.021 mmol) under N₂ at 25° C. Then the mixture was stirred at 25° C. for 2 h. LCMS showed the reaction was completed.

The reaction was quenched by water (20 mL) slowly, extracted with EA (20 mL*3). The combined organic layers were washed with brine (40 mL), dried over sodium sulfate, filtered and concentrated. The residue was purified by combi-flash with DCM/MeOH (10:1) to afford compound 140 (10 mg, 13% yield) as a yellow solid. ¹H NMR (300 MHz, CD₃OD) δ 6.96-6.88 (m, 2H), 6.61-6.54 (m, 2H), 3.58-3.50 (m, 1H), 3.15-3.01 (m, 1H), 2.29-1.87 (m, 2H), 1.66-1.39 (m, 16H), 1.20 (m, 3H), 0.91 (d, J=12 Hz, 6H). MS (m/z) 329.3 [M−H].

### Compound 141

### 4-(+ (4,4-dimethylcyclohexyl)phenyl)amino)pyrrolidin-2-one (1+1)

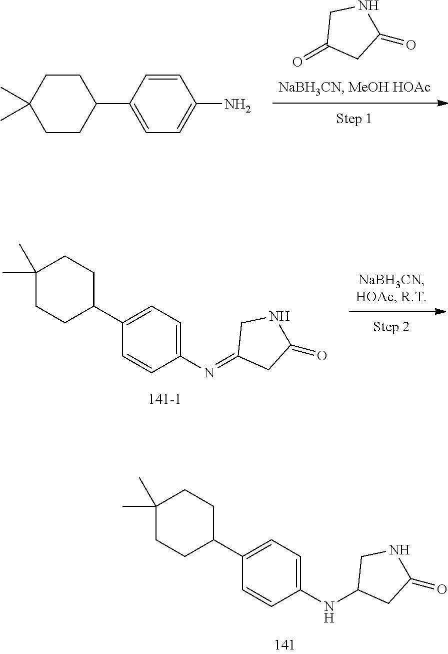

Step 1. Preparation of (Z)-4-((4-(4,4-dimethylcyclohexyl)phenyl)imino)pyrrolidin-2-one (141-1)

A mixture of 4-(4,4-dimethylcyclohexyl)aniline (100 mg, 0.49 mmol), pyrrolidine-2,4-dione (63.4 mg, 0.64 mmol) and NaBH₃CN (61.8 mg, 0.98 mmol) in MeOH (10 mL) and HOAc (1 drop) was stirred overnight at 50° C. After cooling, excess MeOH was removed under vacuum, the residual oil was extracted three times with ethyl acetate (20 mL) and water (10 mL). Organic layers were combined, solvent was removed under vacuum and the crude was purified through silica gel chromatography (PE:EA=10:1) to give the imine intermediate (98 mg, 55.7%) as oil. Mass (m/z): 285.2 [M+H]⁺.

Step 2. Preparation of 4-((4-(4,4-dimethylcyclohexyl)phenyl)amino)pyrrolidin-2-one (141)

NaBH₃CN (15.5 mg, 0.25 mmol) was added to a solution of (Z)-4-((4-(4,4-dimethylcyclohexyl)phenyl)imino)pyrrolidin-2-one (70 mg, 0.25 mmol) in HOAc (10 mL). The mixture was stirred at 25° C. for overnight. After the reaction was completed, the reaction solution was washed with water, solvent was removed under vacuum and the residue was purified by prep-HPLC (column-Xbridge-C18 150×21.2 mm, 5 μm; Mobile phase: ACN-H₂O (0.1% FA), 40%-70%) to afford the desired product 141 (8 mg) as a white solid. ¹H NMR (400 MHZ, DMSO-d₆) δ 8.19 (s, 1H), 7.13 (t, 4H), 7.03 (t, 4H), 3.94 (s, 2H), 3.81 (s, 2H), 3.55 (s, 2H), 2.88 (s, 3H), 2.37-2.30 (m, 1H), 1.63-1.51 (m, 4H), 1.45 (d, 2H), 1.35-1.26 (m, 2H), 0.95 (d, 6H). Mass (m/z): 287.3 [M+H]⁺.

### Compound 142

### N1-(4-butoxyphenyl)cyclohexane-1,4-diamine

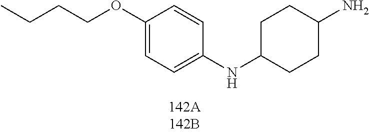 [SMILES: CCCCOc1ccc(NC2CCC(N)CC2)cc1]

The title compounds 142A (17.1 mg) as a white solid and 142B (20.7 mg) as a white solid were prepared from tert-butyl (4-((4-butoxyphenyl)amino)cyclohexyl)carbamate (200 mg, 0.55 mmol) and HCl in 1,4-dioxane (10 mL, 4N) according to the procedure for 24. 142A: ¹H NMR (400 MHZ, DMSO-d₆) δ 8.42 (s, 1H), 6.68 (d, J=8.9 Hz, 2H), 6.50 (d, J=8.9 Hz, 2H), 3.81 (t, J=6.5 Hz, 2H), 3.01 (d, J=11.2 Hz, 1H), 2.87 (d, J=11.1 Hz, 1H), 1.94 (dd, J=23.9, 11.9 Hz, 4H), 1.66-1.58 (m, 2H), 1.48-1.31 (m, 4H), 1.12 (d, J=12.7 Hz, 2H), 0.91 (t, J=7.4 Hz, 3H). MS (m/z) 263.2 [M+H]⁺. HPLC: Rt: 3.789 min (Column: XBRIDGE 2.1*50 mm, 3.5 um; Mobile Phase: H₂O (0.05% TFA)/ACN (0.05% TFA), ACN from 0% to 60% over 7 minutes, 7-8 min, ACN from 60% to 100%; 0.8 mL/min). 142B: ¹H NMR (400 MHZ, DMSO-d₆)) δ 8.42 (s, 1H), 6.69 (d, J=8.9 Hz, 2H), 6.54 (d, J=8.7 Hz, 2H), 3.81 (t, J=6.5 Hz, 2H), 3.34 (s, 1H), 3.00 (s, 1H), 1.74-1.53 (m, 11H), 1.40 (dd, J=15.0, 7.4 Hz, 2H), 0.91 (t, J=7.4 Hz, 3H). MS (m/z) 263.2 [M+H]⁺. HPLC: Rt: 3.887 min (Column: XBRIDGE 2.1*50 mm, 3.5 μm; Mobile Phase: H₂O (0.05% TFA)/ACN (0.05% TFA), ACN from 0% to 60% over 7 minutes, 7-8 min, ACN from 60% to 100%; 0.8 mL/min).

### Compound 143

### (R)—N-((4-((4-(tert-butyl)phenyl)amino)cyclohexyl)methyl)-2-oxoimidazolidine-4-carboxamide

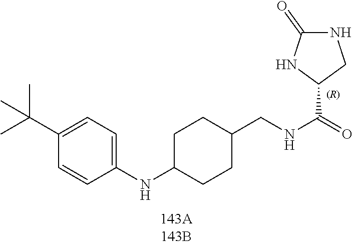

The title compound 143A, 143B was prepared according to the procedure for compound 1. The crude residue was purified by preparative TLC (MeOH: Dichloromethane=1:10) to afford 10.4 mg of compound 143A in 18.6% yield as a light yellow solid and 5.1 mg of 143B in 5.0% yield as a light yellow solid. 143A: ¹H NMR (301 MHZ, DMSO-d₆) δ 7.94 (t, J=5.6 Hz, 1H), 7.46-6.94 (m, 5H), 6.53 (s, 1H), 6.34 (s, 1H), 4.08-4.03 (m, 1H), 3.54 (t, J=9.3 Hz, 1H), 3.47-3.40 (m, 1H), 3.22-3.03 (m, 3H), 1.72-1.39 (m, 8H), 1.26 (s, 6H). Mass (m/z): 373.3 [M+H]⁺. HPLC: Rt-0.334 mins (Agilent, poroshell 120, SB—C18 2.7 μm, 4.6×50 mm, ACN/Water (0.1% FA)=5%-5%-95%-95%-95%-5%, 0-0.5 min-10 min-10.5 min-12.0 min). 143B: ¹H NMR (301 MHz, DMSO-d₆) δ 7.89 (t, J=5.9 Hz, 1H), 7.45-6.94 (m, 5H), 6.51 (s, 1H), 6.33 (s, 1H), 4.04 (dd, J=9.7, 6.3 Hz, 1H), 3.53 (t, J=9.3 Hz, 1H), 3.30-3.14 (m, 2H), 2.94 (t, J=6.3 Hz, 2H), 1.92 (d, J=12.0 Hz, 2H), 1.73 (d, J=12.7 Hz, 2H), 1.50-1.32 (m, 2H), 1.26 (s, 6H), 0.95 (q, J=12.7 Hz, 2H). Mass (m/z): 373.3 [M+H]⁺. HPLC: Rt-0.238 mins (Agilent, poroshell 120, SB—C18 2.7 μm, 4.6×50 mm, ACN/Water (0.1% FA)=5%-5%-95%-95%-95%-5%, 0-0.5 min-10 min-10.5 min-12.0 min).

### Compound 144

### N-((4-((4-(tert-butyl)phenyl)amino)cyclohexyl)methyl)-5-oxopyrrolidine-3-carboxamide

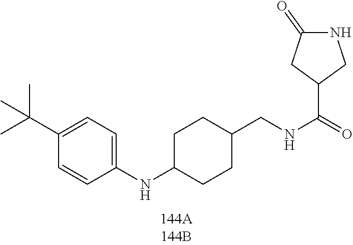 [SMILES: CC(C)(C)c1ccc(NC2CCC(CNC(=O)C3CNC(=O)C3)CC2)cc1]

The title compound 144A and 144B were prepared according to the procedure for compound 1. The crude residue was purified by preparative TLC (MeOH: Dichloromethane=1:10) to afford 3.7 mg of compound 144A in 6.6% yield as a light yellow solid and 4.6 mg of 144B in 8.3% yield as a light yellow solid. 144A: ¹H NMR (301 MHz, DMSO-d₆) δ 8.01 (t, J=5.7 Hz, 1H), 7.59 (s, 1H), 7.35 (s, 2H), 7.14-6.86 (m, 3H), 3.50-3.33 (m, 3H), 3.23-3.03 (m, 4H), 2.28 (d, J=8.3 Hz, 2H), 1.66-1.54 (m, 4H), 1.50-1.39 (m, 4H), 1.25 (s, 9H). Mass (m/z): 373.3 [M+H]⁺. HPLC: Rt-0.372 mins (Agilent, poroshell 120, SB—C18 2.7 μm, 4.6×50 mm, ACN/Water (0.1% FA)=5%-5%-95%-95%-95%-5%, 0-0.5 min-10 min-10.5 min-12.0 min). 144B: ¹H NMR (301 MHz, DMSO-d₆) δ 7.98 (t, J=5.7 Hz, 1H), 7.58 (s, 1H), 7.44-7.30 (m, 2H), 7.12-6.83 (m, 3H), 3.38 (t, J=8.8 Hz, 1H), 3.27-3.08 (m, 4H), 2.92 (t, J=6.3 Hz, 2H), 2.26 (d, J=8.4 Hz, 2H), 1.92 (d, J=12.0 Hz, 2H), 1.73 (d, J=12.7 Hz, 2H), 1.40-1.15 (m, 11H), 1.00-0.90 (m, 2H). Mass (m/z): 373.3 [M+H]⁺. HPLC: Rt-0.173 mins (Agilent, poroshell 120, SB—C18 2.7 4.6x50 μm, mm, ACN/Water (0.1% FA)=5%-5%-95%-95%-95%-5%, 0-0.5 min-10 min-10.5 min-12.0 min).

### Compound 145

### 3-((4-(4,4-dimethylcyclohexyl)phenyl)amino)cyclobutane-1-carboxamide

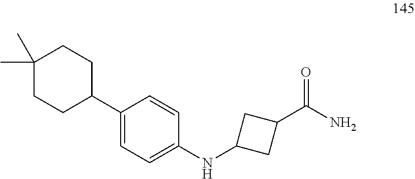 [SMILES: CC1(C)CCC(c2ccc(NC3CC(C(N)=O)C3)cc2)CC1]

The title compound 145 (8.7 mg) was prepared in a total yield of 15.8% as a white solid with 7:3 mixture by ¹H NMR from 3-((4-(4,4-dimethylcyclohexyl)phenyl)amino)cyclobutane-1-carboxylic acid (55 mg, 0.18 mmol), CDI (59 mg, 0.36 mmol) and NH₃ H₂O (0.5 mL) according to the procedure for 118-5. 1¹H NMR (301 MHZ, DMSO-d₆) δ 7.24-7.19 (m, 1H), 6.97-6.90 (m, 2H), 6.78-6.72 (m, 1H), 6.43 (d, J=8.4 Hz, 0.8 H), 6.38 (d, J=8.4 Hz, 1.2H), 5.66 (d, J=6.4 Hz, 0.6 H), 5.63 (d, J=6.4 Hz, 0.4H), 3.89-3.82 (m, 0.6H), 3.69-3.63 (m, 0.4H), 2.97-2.84 (m, 1H), 2.48-2.34 (m, 4H), 2.03-1.89 (m, 3H), 1.56-1.37 (m, 6H), 0.93 (d, J=6.4 Hz, 6H). Mass (m/z): 301.3 [M+H]⁺.

### Compound 146

### 3-((4-(4,4-dimethylcyclohexyl)phenyl)amino)cyclopentane-1-carboxamide

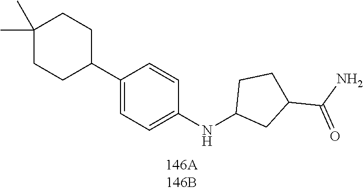 [SMILES: CC1(C)CCC(c2ccc(NC3CCC(C(N)=O)C3)cc2)CC1]

The title compound 146A and 146B were prepared according to the procedure for compound 118-5. The crude residue was purified by preparative TLC (MeOH: Dichloromethane=1:10) to afford compound 146A in 61.4% yield as a light yellow solid and 146B in 47.3% yield as a light yellow solid. 146A: ¹H NMR (400 MHZ, DMSO-d₆) δ 7.21 (s, 1H), 6.90 (d, J=8.4 Hz, 2H), 6.67 (s, 1H), 6.43 (d, J=7.6 Hz, 2H), 5.30 (s, 1H), 3.73-3.62 (m, 1H), 2.69 (p, J=8.1 Hz, 1H), 2.21-2.13 (m, 1H), 1.96-1.82 (m, 3H), 1.65-1.57 (m, 2H), 1.52-1.35 (m, 8H), 1.28-1.21 (m, 2H), 0.90 (d, J=8.8 Hz, 6H). Mass (m/z): 315.3 [M+H]⁺. HPLC: Rt=1.198 mins (Agilent, poroshell 120, SB—C_{18 2.7} μm, 4.6x50 mm, ACN/Water (0.1% FA)=5%-5%-95%-95%-95%-5%, 0-0.5 min-10 min-10.5 min-12.0 min). 146B: ¹H NMR (400 MHz, DMSO-d₆) δ 7.21 (s, 1H), 6.90 (d, J=8.0 Hz, 2H), 6.67 (s, 1H), 6.43 (d, J=8.0 Hz, 2H), 5.30 (s, 1H), 3.72-3.65 (m, 1H), 2.69 (p, J=8.0 Hz, 1H),2.10-2.20 (m, 2H), 2.01-1.82 (m, 3H), 1.66-1.57 (m, 2H), 1.49-1.35 (m, 7H), 1.27-1.20 (m, 2H), 0.90 (d, J=8.8 Hz, 6H). Mass (m/z): 315.3 [M+H]⁺. HPLC: Rt=1.807 mins (Agilent, poroshell 120, SB—C18 2.7 μm, 4.6×50 mm, ACN/Water (0.1% FA)=5%-5%-95%-95%-95%-5%, 0-0.5 min-10 min-10.5 min-12.0 min).

### Compound 147

### N1-(4-(tert-butyl)phenyl)-N4-methylcyclohexane-1,4-diamine

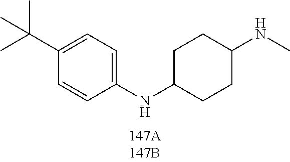 [SMILES: CNC1CCC(Nc2ccc(C(C)(C)C)cc2)CC1]

The title compound 147A and 147B were prepared according to the procedure for compound 118. The crude residue was purified by preparative TLC (MeOH: Dichloromethane=1:5) to afford compound 147A in 27.5% yield as a light yellow solid and 147B in 7.7% yield as a light yellow solid. 147A: ¹H NMR (400 MHZ, DMSO-d₆) δ 8.70 (s, 2H), 7.05 (d, J=8.6 Hz, 3H), 6.50 (d, J=8.4 Hz, 2H), 5.19 (d, J=5.6 Hz, 1H), 3.45-3.36 (m, 1H), 3.00-2.93 (m, 1H), 2.46 (s, 3H), 1.82-1.70 (m, 6H), 1.59-1.49 (m, 2H), 1.17 (s, 9H). Mass (m/z): 261.3 [M+H]⁺. HPLC: Rt=0.423 mins (Agilent, poroshell 120, SB—C_{18 2.7} μm, 4.6x50 mm, ACN/Water (0.1% FA)=5%-5%-95%-95%-95%-5%, 0-0.5 min-10 min-10.5 min-12.0 min). 147B: 1H NMR (400 MHz, DMSO-d₆) δ 7.11-7.00 (m, 2H), 6.50-6.41 (m, 2H), 5.10 (d, J=8.2 Hz, 1H), 3.44-3.27 (m, 2H), 3.12-3.03 (m, 2H), 6.50-6.41 (m, 2H), 2.30-2.18 (m, 4H), 1.97-1.84 (m, 4H), 1.20 (s, 9H), 1.13-0.98 (m, 4H). Mass (m/z): 261.3 [M+H]⁺. HPLC: Rt=0.318 mins (Agilent, poroshell 120, SB—C18 2.7 μm, 4.6×50 mm, ACN/Water (0.1% FA)=5%-5%-95%-95%-95%-5%, 0-0.5 min-10 min-10.5 min-12.0 min).

### Compound 148

### (R)—N-((4-((4-(4,4-dimethylcyclohexyl)phenyl)amino)cyclohexyl)methyl)-2,6-dioxohexahydropy rimidine-4-carboxamide

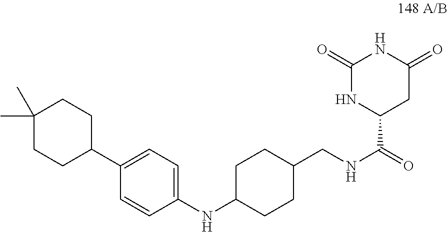 [SMILES: CC1(C)CCC(c2ccc(NC3CCC(CNC(=O)[C@H]4CC(=O)NC(=O)N4)CC3)cc2)CC1]

The title compound 148 was prepared from N1-(4-(tert-butyl)phenyl)cyclohexane-1,4-diamine (18.6 mg, 0.06 mmol) according to the procedure for compound 1. The mixture was purified by preparative HPLC (Column: X Select-CSH-Prep 5 μm OBD, 19*150 mm; ACN/water (0.5% TFA)=20%-45%-95%-95%-10%, 0 min-12 min-12.5 min-13.5 min-15 min) to afford compound 148A (Rt=9.79 min) as a sandy brown solid and compound 148B (Rt=10.42 min) as a sandy brown solid. 148A (7.0 mg, 26%): ¹H NMR (400 MHZ, DMSO-d₆) δ 9.98 (s, 1H), 8.00 (s, 1H), 7.54 (s, 1H), 7.29-7.03 (m, 4H), 6.95 (s, 1H), 3.92 (m, 1H), 3.17 (m, 1H), 2.88 (m, 2H), 2.66-2.54 (m, 2H), 2.29 (m, 1H), 1.97-1.85 (m, 3H), 1.68 (m, 2H), 1.56-1.22 (m, 12H), 0.92 (s, 3H), 0.89 (s, 3H). Mass (m/z): 455.2 [M+H]⁺. 148B (14.1 mg, 53%): ¹H NMR (400 MHZ, DMSO-d₆) δ 9.97 (s, 1H), 8.01 (s, 1H), 7.55 (s, 1H), 7.29-6.84 (m, 5H), 3.93 (m, 1H), 3.01 (m, 2H), 2.80 (m, 1H), 2.54 (m, 1H), 2.42 (m, 1H), 2.29 (m, 1H), 1.63-1.46 (m, 8H), 1.44-1.33 (m, 6H), 1.31-1.23 (m, 3H), 0.92 (s, 3H), 0.89 (s, 3H). Mass (m/z): 455.2 [M+H]⁺.

### Compound 149

### N1-(4-(tert-butyl)phenyl)cyclohexane-1,3-diamine

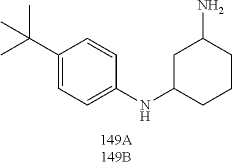 [SMILES: CC(C)(C)c1ccc(NC2CCCC(N)C2)cc1]

The title compound 149 was prepared from tert-butyl (3-((4-(tert-butyl)phenyl)amino)cyclohexyl)carbamate (520 mg, 1.5 mmol) according to the procedure for compound 88. The mixture was purified by preparative HPLC (Column: X Select-CSH-Prep 5 μm OBD, 19*150 mm; ACN/water (0.5% TFA)=10%-32%-95%-95%-10%, 0 min-10 min-10.5 min-11.5 min-13 min) to afford compound 149A (Rt=6.88 min) as a white solid and compound 149B (Rt=9.25 min) as a white solid. 149A (79.8 mg, 21%): ¹H NMR (400 MHZ, DMSO-d₆) δ 7.33 (d, J=8.4 Hz, 2H), 6.94 (d, J=8.4 Hz, 2H), 3.39 (m, 1H), 3.09 (m, 1H), 2.25-1.75 (m, 4H), 1.44-1.28 (m, 4H), 1.24 (s, 9H). Mass (m/z): 247.2 [M+H]⁺. 149B (188.5 mg, 51%): ¹H NMR (400 MHZ, DMSO-d₆) δ 7.28 (d, J=8.4 Hz, 2H), 6.89 (d, J=8.4 Hz, 2H), 3.77 (m, 1H), 3.46 (m, 1H), 2.13-1.32 (m, 8H), 1.23 (s, 9H). Mass (m/z): 247.2 [M+H]⁺.

### Compound 150

### N-(4-(tert-butyl)phenyl)piperidin-3-amine

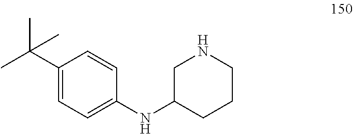 [SMILES: CC(C)(C)c1ccc(NC2CCCNC2)cc1]

The title compound 150 (151.2 mg) was prepared from tert-butyl 3-((4-(tert-butyl)phenyl)amino)piperidine-1-carboxylate (498 mg, 1.5 mmol) according to the procedure for compound 88. ¹H NMR (400 MHZ, DMSO-d₆) δ 7.09 (d, J=8.4 Hz, 2H), 6.95 (s, 1H), 6.58 (d, J=8.4 Hz, 2H), 5.52 (s, 1H), 3.59 (s, 1H), 3.29-3.06 (m, 2H), 2.74-2.56 (m, 2H), 1.98-1.38 (m, 4H), 1.20 (s, 9H). Mass (m/z): 233.4 [M+H]⁺.

### Compound 151

### 3-(+-(+++-dimethylcyclohexyl)phenyl)amino)cyclopentan-1-ol

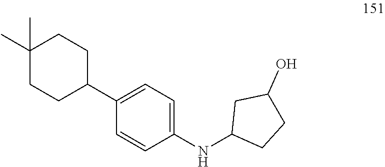 [SMILES: CC1(C)CCC(c2ccc(NC3CCC(O)C3)cc2)CC1]

The title compound 151 (32.1 mg) was prepared in a total yield of 76% as a chocolate solid from 4-(4,4-dimethylcyclohexyl)aniline (31 mg, 0.15 mmol) and 3-hydroxycyclopentan-1-one (20 mg, 0.2 mmol) according to the procedure for compound 4. ¹H NMR (400 MHZ, DMSO-d₆) δ 6.93 (d, J=8.4 Hz, 2H), 6.47 (d, J=8.4 Hz, 2H), 5.40 (s, 1H), 4.49 (s, 1H), 4.19 (m, 1H), 3.79 (m, 1H), 2.20 (m, 1H), 2.12-1.83 (m, 3H), 1.55-1.27 (m, 11H), 0.93 (s, 3H), 0.91 (s, 3H). Mass (m/z): 288.4 [M+H]⁺.

### Compound 152

### N1-(4-(tert-butyl)phenyl)cyclohexane-1,2-diamine

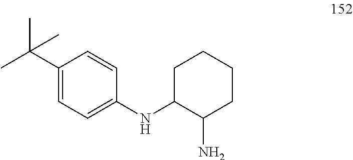 [SMILES: CC(C)(C)c1ccc(NC2CCCCC2N)cc1]

The title compound 152 (271.5 mg) was prepared in a total yield of 67% as a yellow solid from 4-(tert-butyl)aniline (223 mg, 1.5 mmol) and tert-butyl (2-oxocyclohexyl)carbamate (426 mg, 2.0 mmol) according to the procedure for compound 24. ¹H NMR (400 MHZ, DMSO-d₆) δ 7.11 (d, J=8.4 Hz, 2H), 6.67 (d, J=8.4 Hz, 2H), 3.32 (m, 1H), 2.95 (m, 1H), 2.26-1.92 (m, 2H), 1.83-1.31 (m, 4H), 1.21 (s, 9H), 1.18-1.07 (m, 2H). Mass (m/z): 247.3 [M+H]⁺.

### Compound 153

### N1-(3-(tert-butyl)phenyl)cyclohexane-1,4-diamine

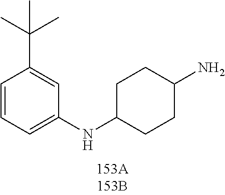 [SMILES: CC(C)(C)c1cccc(NC2CCC(N)CC2)c1]

The title compound 153A (Rt=3.65 min; 45.3 mg) was prepared in a yield of 27.4% as a white powder and compound 153B (Rt=4.28 min; 36.7 mg) was prepared in a yield of 22.2% as a white powder 3-(tert-butyl)aniline (100 mg, 0.67 mmol), tert-butyl (4-oxocyclohexyl)carbamate (214 mg, 1.01 mmol) and according to the procedure for 20 which were prepared by Prep-HPLC (Column: X Select-CSH-Prep 5 μm OBD, 19*150 mm; ACN/water (0.5% TFA)=5%-5%-95%-95%-5%, 0 min-1 min-10 min-11 min-15.0 min). 153A: ¹H NMR (400 MHZ, DMSO-d₆) δ 7.85 (s, 3H), 7.12 (s, 1H), 6.84 (s, 2H), 6.65 (s, 1H), 3.21 (s, 1H), 3.06-2.93 (m, 1H), 2.07-1.89 (m, 4H), 1.38 (qd, J=12.0, 11.3, 6.1 Hz, 2H), 1.25 (d, J=6.3 Hz, 2H), 1.24-1.20 (m, 9H). 153: 1¹H NMR (400 MHZ, DMSO-d₆) δ 7.83 (s, 2H), 7.13-6.93 (m, 1H), 6.60 (d, J=84.7 Hz, 3H), 3.47 (s, 1H), 3.14 (s, 1H), 1.88-1.56 (m, 8H), 1.24 (d, J=1.2 Hz, 9H). Mass (m/z): 247.6 [M+H]⁺.

### Compound 154

### (R)-4-(4-((4-(4,4-dimethylcyclohexyl)phenyl)amino)piperidine-1-carbonyl) imidazolidin-2-one

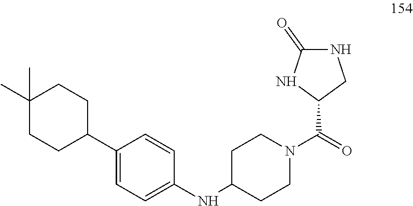 [SMILES: CC1(C)CCC(c2ccc(NC3CCN(C(=O)[C@H]4CNC(=O)N4)CC3)cc2)CC1]

The title compound 154 (21.2 mg) was prepared in a yield of 71.2% as a white powder from N-(4-(4,4-dimethylcyclohexyl)phenyl)piperidin-4-amine (21.4 mg, 0.07 mmol), (R)-2-oxoimidazolidine-4-carboxylic acid (11 mg, 0.08 mmol) and according to the procedure for 1. ¹H NMR (400 MHZ, DMSO-d₆) δ 7.22 (s, 1H), 6.94 (d, J=8.1 Hz, 2H), 6.67 (s, 1H), 6.52 (d, J=8.0 Hz, 2H), 6.26 (d, J=6.6 Hz, 1H), 4.55-4.60 (m, 1H), 4.18 (s, 1H), 2.40-2.45 (m, 2H), 1.90 (s, 2H), 1.52 (s, 4H), 1.49-1.37 (m, 9H), 1.08-1.13 (m, 1H), 0.94 (s, 3H), 0.92 (s, 3H). Mass (m/z): 399.3 [M+H]⁺.

### Compound 155

### N1-(4-(4,4-dimethylcyclohexyl)phenyl)cyclopentane-1,3-diamine

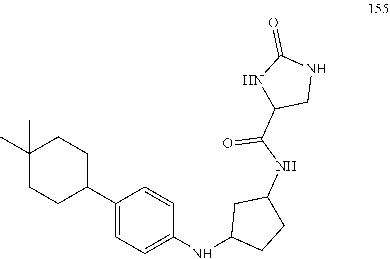 [SMILES: CC1(C)CCC(c2ccc(NC3CCC(NC(=O)C4CNC(=O)N4)C3)cc2)CC1]

The title compound 155 (76.4 mg) was prepared in a yield of 33.0% as a white powder with 2:1 mixture by ¹H NMR from N1-(4-(4,4-dimethylcyclohexyl)phenyl)cyclopentane-1,3-diamine (166 mg, 0.58 mmol), (R)-2-oxoimidazolidine-4-carboxylic acid (83 mg, 0.64 mmol) and according to the procedure for 1. ¹H NMR (400 MHZ, DMSO-d₆) δ 7.93 (s, 1H), 7.20 (d, J=63.2 Hz, 4H), 6.37 (d, J=61.1 Hz, 2H), 4.28-4.21 (m, 1H), 4.00 (dd, J=9.8, 6.2 Hz, 2H), 3.50 (td, J=9.0, 6.7 Hz, 1H), 3.24-3.11 (m, 1H), 2.38 (s, 1H), 2.09-1.97 (m, 2H), 1.87 (d, J=8.7 Hz, 1H), 1.76 (dt, J=13.8, 7.3 Hz, 1H), 1.57 (d, J=11.0 Hz, 5H), 1.43 (d, J=12.5 Hz, 3H), 1.31 (d, J=14.5 Hz, 1H), 0.95 (s, 3H), 0.92 (s, 3H). Mass (m/z): 399.4 [M+H]⁺.

### Compound 156

### (R)—N-(4-((4-(4,4-dimethylcyclohexyl) phenyl)amino)cyclohexyl)-2-oxoimidazolidine-4-carboxamide

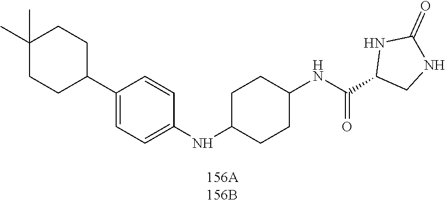 [SMILES: CC1(C)CCC(c2ccc(NC3CCC(NC(=O)[C@H]4CNC(=O)N4)CC3)cc2)CC1]

The title compound 156A (Rt-6.19 min; 4.5 mg) was prepared in a yield of 5.7% as a white powder and compound 156B (Rt=7.19 min; 10 mg) was prepared in a yield of 12.6% as a white powder from N1-(4-(4,4-dimethylcyclohexyl)phenyl)cyclohexane-1,4-diamine (57 mg, 0.19 mmol), (R)-2-oxoimidazolidine-4-carboxylic acid (27 mg, 0.21 mmol) according to the procedure for 1 which were prepared by Prep-HPLC (Column: X Select-CSH-Prep 5 μm OBD, 19*150 mm; ACN/water (0.5% TFA)=25%-43%-95%-95%-10%, 0 min-10.0 min-10.5 min-11.5 min-13.0 min). 156A: ¹H NMR (400 MHZ, DMSO-d₆) δ 7.77 (d, J=7.8 Hz, 1H), 7.23 (s, 2H), 7.14-6.91 (s, 1H), 6.48 (s, 1H), 6.30 (s, 1H), 4.03 (dd, J=9.7, 6.0 Hz, 2H), 3.55-3.48 (m, 2H), 3.17 (dd, J=8.9, 6.1 Hz, 1H), 2.00 (q, J=6.8, 6.2 Hz, 1H), 1.93 (d, J=10.8 Hz, 2H), 1.82 (d, J=10.8 Hz, 2H), 1.64-1.51 (m, 4H), 1.45 (d, J=12.3 Hz, 3H), 1.32 (t, J=9.0 Hz, 4H), 0.97 (s, 3H), 0.94 (s, 3H). Mass (m/z): 413.4 [M+H]⁺. 156B: ¹H NMR (400 MHZ, DMSO-d₆) δ 7.64-7.50 (m, 1H), 7.21 (s, 2H), 7.00 (s, 1H), 6.51 (s, 1H), 6.31 (s, 1H), 4.09 (dd, J=9.7, 5.9 Hz, 1H), 3.72 (s, 1H), 3.52 (t, J=9.3 Hz, 1H), 3.39 (d, J=8.5 Hz, 1H), 3.20 (dd, J=8.9, 5.9 Hz, 1H), 1.98 (p, J=7.0, 6.5 Hz, 1H), 1.74 (s, 2H), 1.69-1.60 (m, 4H), 1.60-1.48 (m, 6H), 1.43 (d, J=12.8 Hz, 2H), 1.31 (d, J=13.7 Hz, 2H), 0.95 (s, 3H), 0.92 (s, 3H). Mass (m/z): 413.4 [M+H]⁺.

### Compound 157

### N-(4-((4-(4,4-dimethylcyclohexyl)phenyl)amino)cyclohexyl)acetamide

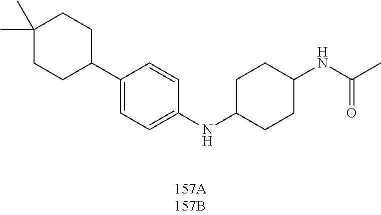 [SMILES: CC(=O)NC1CCC(Nc2ccc(C3CCC(C)(C)CC3)cc2)CC1]

The title compound 157A (Rt=6.02 min; 18.7 mg) was prepared in a yield of 16.4% as a white powder and compound 157B (Rt=6.34 min; 25.4 mg) was prepared in a yield of 22.8% as a white powder from N1-(4-(4,4-dimethylcyclohexyl)phenyl)cyclohexane-1,4-diamine (100 mg, 0.33 mmol), acetic acid (30 mg, 0.50 mmol) and according to the procedure for 1 which were prepared by Prep-HPLC (Column: X Select-CSH-Prep 5 μm OBD, 19*150 mm; ACN/water (0.5% TFA)=5%-5%-95%-95%-5%, 0 min-1 min-10 min-11 min-15.0 min). 175A: ¹H NMR (400 MHZ, DMSO-d₆) δ 7.77 (d, J=7.6 Hz, 1H), 7.29 (s, 2H), 7.13 (s, 2H), 3.53-3.39 (m, 1H), 2.40 (s, 1H), 1.99 (q, J=6.9, 6.4 Hz, 1H), 1.95-1.77 (m, 4H), 1.75 (s, 3H), 1.66-1.52 (m, 4H), 1.49-1.40 (m, 3H), 1.39-1.26 (m, 4H), 1.17 (d, J=12.7 Hz, 1H), 0.95 (s, 3H), 0.92 (s, 3H). Mass (m/z): 343.5 [M+H]⁺. 175B: ¹H NMR (400 MHz, DMSO-d₆) δ 7.74 (d, J=5.9 Hz, 1H), 7.31 (s, 2H), 7.14 (s, 2H), 3.69 (q, J=4.7 Hz, 1H), 3.40 (d, J=6.8 Hz, 1H), 2.41 (s, 1H), 1.83 (s, 3H), 1.76 (q, J=7.3, 5.8 Hz, 2H), 1.67 (q, J=5.7 Hz, 4H), 1.63-1.56 (m, 4H), 1.56-1.41 (m, 4H), 1.37-1.27 (m, 2H), 0.97 (s, 3H), 0.94 (s, 3H). Mass (m/z): 343.5 [M+H]⁺.

### Compound 158

### (R)—N-(2-((4-(4,4-dimethylcyclohexyl)phenyl)amino)ethyl)-2-oxoimidazolidine-4-carboxamide

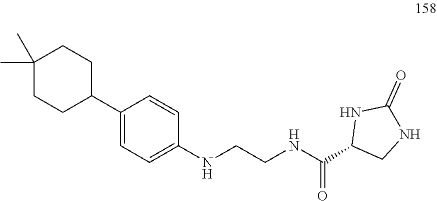 [SMILES: CC1(C)CCC(c2ccc(NCCNC(=O)[C@H]3CNC(=O)N3)cc2)CC1]

The title compound 158 (7.6 mg) was prepared in a yield of 12.7% as a white powder from N1-(4-(4,4-dimethylcyclohexyl)phenyl)ethane-1,2-diamine (41 mg, 0.17 mmol), (R)-2-oxoimidazolidine-4-carboxylic acid (32 mg, 0.25 mmol) according to the procedure for 1. 1¹H NMR (400 MHZ, DMSO-d₆) δ 8.04 (t, J=5.8 Hz, 1H), 6.98-6.93 (m, 2H), 6.55-6.48 (m, 3H), 6.37-6.30 (m, 1H), 4.06 (ddd, J=9.8, 6.4, 1.9 Hz, 1H), 3.55 (ddd, J=9.9, 8.9, 1.1 Hz, 1H), 3.28-3.23 (m, 2H), 3.19 (ddd, J=8.9, 6.4, 1.3 Hz, 1H), 3.06 (q, J=6.5 Hz, 2H), 2.23 (tt, J=10.3, 5.1 Hz, 1H), 1.52 (td, J=12.0, 11.1, 3.3 Hz, 4H), 1.43 (d, J=13.1 Hz, 3H), 1.30 (dd, J=12.2, 5.3 Hz, 1H), 0.95 (s, 3H), 0.93 (s, 3H). Mass (m/z): 359.3 [M+H]⁺.

### Compound 159

### 4-((4-(4,4-dimethylcyclohexyl)phenyl)amino)cyclohexane-1-carboxamide

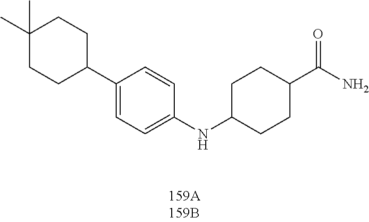 [SMILES: CC1(C)CCC(c2ccc(NC3CCC(C(N)=O)CC3)cc2)CC1]

The title compound 159 was prepared from 4-(4,4-dimethylcyclohexyl)aniline (100 mg, 0.493 mmol), 4-oxocyclohexane-1-carboxamide (104 mg, 0.739 mmol), AcOH (0.1 mL), NaBH (OAc) 3 (209 mg, 0.986 mmol) and DCE (10 mL) according to the procedure for 1-1, which was purified by prep HPLC (Column: X Select-CSH-Prep 5 μm OBD, 19*150 mm; ACN/water (0.5% TFA)=25%-50%-95%-95%-10%, 0 min-10 min-10.5 min-11.5 min-13 min) to give the desired product 159A (Rt=7.1 min) as a white solid (20.5 mg, 12.7%) and 159B (Rt=8.8 min) as a white solid (32.4 mg, 20.0%). 159A: ¹H NMR (400 MHZ, DMSO-d₆) δ 7.23 (s, 1H), 6.90 (d, J=8.0 Hz, 2H), 6.55 (s, 1H), 6.23 (d, J=7.2 Hz, 2H), 5.22 (s, 1H), 3.72-3.62 (m, 1H), 2.71 (p, J=8.0 Hz, 1H), 2.26-2.13 (m, 1H), 1.96-1.82 (m, 3H), 1.65-1.57 (m, 2H), 1.51-1.33 (m, 10H), 1.25-1.21 (m, 2H), 0.93 (d, J=8.0 Hz, 6H). Mass (m/z): 328.3 [M+H]⁺. 159B: ¹H NMR (400 MHZ, DMSO-d₆) δ 7.21 (s, 1H), 6.90 (d, J=8.0 Hz, 2H), 6.67 (s, 1H), 6.43 (d, J=8.0 Hz, 2H), 5.28 (s, 1H), 3.72-3.65 (m, 1H), 2.67 (p, J=8.0 Hz, 1H), 1.65-1.56 (m, 2H), 2.01-1.82 (m, 3H), 1.66-1.57 (m, 2H), 1.48-1.34 (m, 9H), 1.27-1.20 (m, 2H), 0.90 (d, J=8.8 Hz, 6H). Mass (m/z): 328.3 [M+H]⁺.

### Compound 160

### N1-(3, 4-difluorophenyl)cyclohexane-1,4-diamine

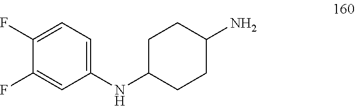 [SMILES: NC1CCC(Nc2ccc(F)c(F)c2)CC1]

The title compound 160 (22.9 mg) was prepared in a total yield of 47.2% as a white solid with 1:2 mixture by ¹H NMR from tert-butyl (4-((3,4-difluorophenyl)amino)cyclohexyl)carbamate (73 mg, 0.215 mmol), TFA (1 mL), and DCM (10 mL) according to the procedure for 24. ¹H NMR (400 MHZ, DMSO-d₆) δ 7.19-6.97 (m, 1H), 6.56 (dddd, J=13.6, 10.0, 6.8, 2.8 Hz, 1H), 6.45-6.26 (m, 1H), 5.75 (dd, J=14.8, 6.8 Hz, 1H), 3.15-2.89 (m, 2H), 1.99 (dt, J=12.8, 4.8 Hz, 2H), 1.75 (tt, J=8.4, 4.0 Hz, 3H), 1.60 (dq, J=15.2, 6.4, 5.6 Hz, 1H), 1.48 (qd, J=13.6, 12.8, 3.6 Hz, 1H), 1.25-1.11 (m, 2H). Mass (m/z): 227.3 [M+H]⁺.

### Compound 161

### N1-(4-chlorophenyl)cyclohexane-1,4-diamine

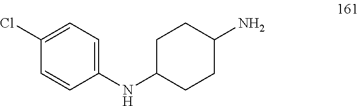 [SMILES: NC1CCC(Nc2ccc(Cl)cc2)CC1]

The title compound 161 (28.6 mg) was prepared in a total yield of 48.5% as a white solid with 1:2 mixture by ¹H NMR from tert-butyl (4-((4-chlorophenyl)amino)cyclohexyl)carbamate (85 mg, 0.262 mmol), TFA (1 mL), and DCM (10 mL) according to the procedure for 24. H NMR (400 MHZ, DMSO-d₆) δ 7.19-7.00 (m, 2H), 6.62 (dd, J=9.5, 3.0 Hz, 2H), 3.38 (t, J=4.0 Hz, 3H), 3.17-3.00 (m, 3H), 2.01 (q, J=9.9, 9.3 Hz, 2H), 1.76 (q, J=5.2, 3.9 Hz, 3H), 1.65-1.55 (m, 1H), 1.53-1.41 (m, 1H). Mass (m/z): 225.3 [M+H]⁺.

### Compound 162

### N1-(2,3-dihydro-1H-inden-5-yl)cyclohexane-1,4-diamine

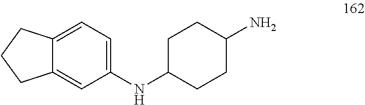 [SMILES: NC1CCC(Nc2ccc3c(c2)CCC3)CC1]

The title compound 162 (32.7 mg) was prepared in a total yield of 53.2% as a white solid with 1:2 mixture by ¹H NMR from tert-butyl (4-((2,3-dihydro-1H-inden-5-yl)amino)cyclohexyl)carbamate (89 mg, 0.27 mmol), TFA (1 mL), and DCM (10 mL) according to the procedure for 24. ¹H NMR (400 MHZ, DMSO-d₆) δ 8.25 (d, J=34.0 Hz, 3H), 6.91 (d, J=8.0 Hz, 1H), 6.50 (s, 1H), 6.41 (d, J=8.4 Hz, 1H), 5.11 (s, 1H), 3.15-2.86 (m, 2H), 2.71 (dt, J=15.2, 7.2 Hz, 4H), 2.00 (dd, J=13.2, 4.0 Hz, 2H), 1.92 (q, J=7.2 Hz, 2H), 1.76 (tt, J=14.0, 8.0 Hz, 3H), 1.59 (td, J=10.0, 4.8 Hz, 1H), 1.52 1.39 (m, 1H). Mass (m/z): 231.3 [M+H]⁺.

### Compound 163

### N1-(pyridin-3-yl)cyclohexane-1,4-diamine

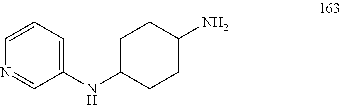 [SMILES: NC1CCC(Nc2cccnc2)CC1]

The title compound 163 (13.6 mg) was prepared in a total yield of 37.8% as a yellow solid with 1:2 mixture by ¹H NMR from tert-butyl (4-(pyridin-3-ylamino)cyclohexyl)carbamate (55 mg, 0.189 mmol), TFA (1 mL), and DCM (10 mL) according to the procedure for 24. ¹H NMR (400 MHz, DMSO-d₆) δ 7.96 (dd, J=24.4, 2.8 Hz, 1H), 7.69 (ddd, J=8.0, 4.4, 1.2 Hz, 1H), 7.05 (dt, J=8.4, 4.8 Hz, 1H), 6.94 (dddd, J=8.4, 6.0, 2.8, 1.2 Hz, 1H), 5.84 (t, J=6.4 Hz, 1H), 3.17-3.02 (m, 1H), 2.90 (d, J=13.2 Hz, 1H), 1.97 (ddd, J=12.4, 7.2, 3.2 Hz, 2H), 1.73 (dq, J=10.0, 5.2 Hz, 3H), 1.60 (ddd, J=13.6, 8.0, 4.0 Hz, 1H), 1.50-1.37 (m, 1H), 1.24-1.09 (m, 2H). Mass (m/z): 192.3 [M+H]⁺.

### Compound 164

### N1-(2,3-dihydrobenzofuran-4-yl)cyclohexane-1,4-diamine

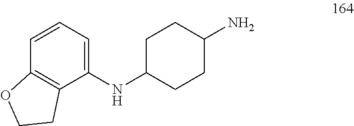 [SMILES: NC1CCC(Nc2cccc3c2CCO3)CC1]

The title compound 164 (64.8 mg) was prepared in a total yield of 89% as a white solid with 1:2 mixture by ¹H NMR from tert-butyl (4-((2,3-dihydrobenzofuran-4-yl)amino)cyclohexyl)carbamate (105 mg, 0.316 mmol), TFA (1 mL), and DCM (10 mL) according to the procedure for 24. ¹H NMR (400 MHZ, DMSO-d₆) δ 6.81 (q, J=7.6 Hz, 1H), 6.07 (d, J=8.4 Hz, 1H), 5.98 (dd, J=16.4, 7.6 Hz, 1H), 4.84-4.37 (m, 3H), 3.18-3.05 (m, 1H), 2.93 (dt, J=41.2, 8.8 Hz, 3H), 2.02-1.90 (m, 2H), 1.84-1.74 (m, 2H), 1.73-1.65 (m, 1H), 1.63-1.54 (m, 1H), 1.50-1.37 (m, 1H), 1.25 (td, J=12.8, 11.2, 4.0 Hz, 1H). Mass (m/z): 233.3 [M+H]⁺.

### Compound 165

### N1-(4-bromophenyl)cyclohexane-1,4-diamine

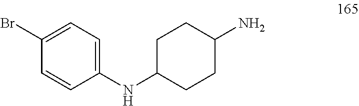 [SMILES: NC1CCC(Nc2ccc(Br)cc2)CC1]

The title compound 165 (106.7 mg) was prepared in a total yield of 83.1% as a white solid with 1:2 mixture by ¹H NMR from tert-butyl (4-((4-bromophenyl)amino)cyclohexyl)carbamate (176 mg, 0.477 mmol), TFA (1 mL), and DCM (10 mL) according to the procedure for 24. ¹H NMR (400 MHZ, DMSO-d₆) δ 7.13 (t, J=8.4 Hz, 2H), 6.56-6.46 (m, 2H), 5.76-5.64 (m, 1H), 3.09-2.85 (m, 2H), 2.00-1.92 (m, 2H), 1.81-1.64 (m, 3H), 1.62-1.51 (m, 1H), 1.50-1.38 (m, 1H), 1.18-1.13 (m, 1H). Mass (m/z): 270.3 [M+H]⁺.

### Compound 166

### N1-(2,3-dihydrobenzofuran-6-yl)cyclohexane-1,4-diamine

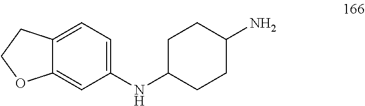 [SMILES: NC1CCC(Nc2ccc3c(c2)OCC3)CC1]

The title compound 166 (78.2 mg) was prepared in a total yield of 69.8% as a yellow solid with 1:2 mixture by ¹H NMR from tert-butyl (4-((2,3-dihydrobenzofuran-6-yl)amino)cyclohexyl)carbamate (160 mg, 0.482 mmol), TFA (1 mL), and DCM (10 mL) according to the procedure for 24. ¹H NMR (400 MHZ, DMSO-d₆) δ 6.83 (dd, J=8.0, 5.6 Hz, 1H), 6.12-5.91 (m, 2H), 5.23 (d, J=7.6 Hz, 1H), 4.37 (td, J=8.4, 1.2 Hz, 2H), 3.06-2.85 (m, 4H), 1.98-1.92 (m, 2H), 1.75-1.67 (m, 3H), 1.58-1.50 (m, 1H), 1.48-1.37 (m, 1H), 1.15-1.03 (m, 1H). Mass (m/z): 233.3 [M+H]⁺.

### Compound 167

### N-(4-(aminomethyl)cyclohexyl)-6-(4,4-dimethylcyclohexyl)pyridin-3-amine

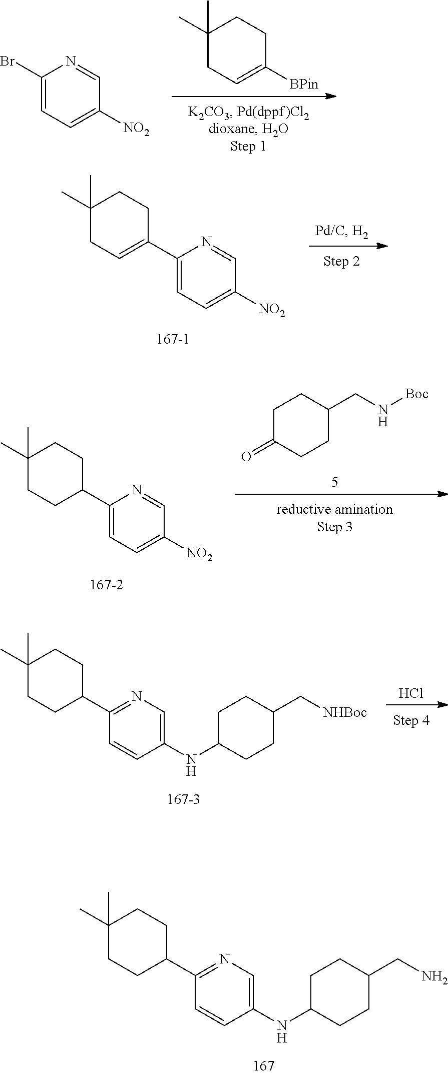

Step 1. Preparation of 2-(4,4-dimethylcyclohex-1-en-1-yl)-5-nitropyridine (167-1)

To a solution of 2-bromo-5-nitropyridine (500 mg, 2.46 mmol), 2-(4,4-dimethylcyclohex-1-en-1-yl)-4,4,5,5-tetramethyl-1,3,2-dioxaborolane (459 mg, 3.69 mmol), K₂CO₃ (1019 mg, 7.39 mmol) in 1,4-dioxane (10 mL) and water (1 mL) was added Pd (dppf) Cl₂ (285 mg, 0.25 mmol). The mixture was stirred with refluxing overnight under N₂ atmosphere. After cooling to ambient temperature, the mixture was concentrated under reduced pressure. The residue was purified by silica gel column to provide compound 167-1 (500 mg, 87.3% yield) as a yellow solid. MS (m/z) 233.2 [M+H]⁺.

Step 2. Preparation of 6-(4,4-dimethylcyclohexyl)pyridin-3-amine (167-2)

Pd/C (200 mg, 10%) was added to a solution of 2-(4,4-dimethylcyclohex-1-en-1-yl)-5-nitropyridine (500 mg, 2.15 mmol) in MeOH (10 mL), and the mixture was allowed to react under H₂ for 16 h. Then it was filtered and solvent was removed under vacuum to give compound 167-2 (390 mg, 88.6% yield) as a yellow solid. MS (m/z) 205.1 [M+H]⁺.

Step 3. Preparation of tert-butyl ((4-((6-(4,4-dimethylcyclohexyl)pyridin-3-yl)amino)cyclohexyl)methyl)carbamate (167-3)

To a solution of compound 4 (200 mg, 0.98 mmol) in MeOH (10 mL) and a drop of AcOH was added compound 167-2 (223 mg, 0.98 mmol). Sodium cyanoborohydride (123 mg, 1.96 mmol) was added and the mixture was stirred at 50° C. for 16 h. LCMS showed the reaction was completed. The reaction was quenched with water (10 mL), and extracted with EA (10 mL*3). The combined organic layers were washed with brine (20 mL), dried over sodium sulfate, filtered and concentrated. The residue was purified by combi-flash with EA/PE (1:3) to afford compound 167-3 (0.4 g, 98.3% yield) as a yellow solid. MS (m/z) 415.9 [M+H]⁺.

Step 4. Preparation of N-(4-(aminomethyl)cyclohexyl)-6-(4,4-dimethylcyclohexyl)pyridin-3-amine (167)

compound 167-3 (200 mg, 0.48 mmol) and HCl in 1,4-dioxane (10 mL, 4N) were placed in a flask stirred at 25° C. for 16 hrs. Excess 1,4-dioxane was distilled under vacuum and the residue was purified by prep-HPLC (column-Xbridge-C18 150×19 mm, 5 μm; Mobile phase: ACN-H₂O (0.1% FA), 2%-20%) to afford 167 (45 mg 29.6%) as a white solid. ¹H NMR (400 MHz, CD₃OD) δ 7.86-7.60 (m, 3H), 3.35 (s, 1H), 2.85 (dd, J=15.4, 7.2 Hz, 2H), 2.78-2.66 (m, 1H), 2.16-2.11 (m, 1H), 1.94-1.90 (m, 1H), 1.83-1.65 (m, 7H), 1.59-1.20 (m, 8H), 1.01 (d, J=16.8 Hz, 6H). MS (m/z) 316.3 [M+H]⁺.

### Compound 168

### N-(4-(tert-butyl)phenyl)-N-(2,2,2-trifluoroethyl)cyclohexane-1,4-diamine

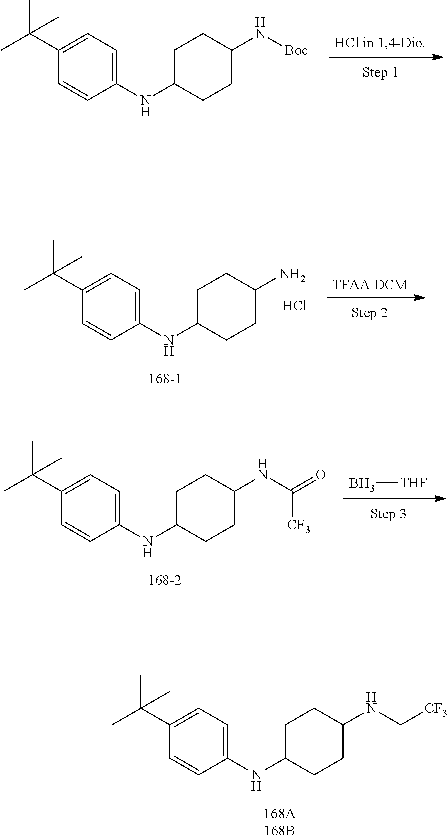

Step 1. Preparation of N-(4-(tert-butyl)phenyl)cyclohexane-1,4-diamine hydrochloride (168-1)

tert-Butyl (4-((4-(tert-butyl)phenyl)amino)cyclohexyl)carbamate (1.2 g, 3.5 mmol) and HCl in 1,4-dioxane (20 mL, 4 N) were placed in a flask and stirred at 25° C. for 18 hrs. The solution was concentrated under vacuum to afford 1 g of N-(4-(tert-butyl)phenyl)cyclohexane-1,4-diamine hydrochloride (yield: 90%). Mass (m/z): 247.3 [M+H]⁺.

Step 2. Preparation of N-(4-((4-(tert-butyl)phenyl)amino)cyclohexyl)-2,2,2-trifluoroacetamide (168-2)

A 100-mL round-bottom flask was charged with 168-1 (400 mg, 1.62 mmol), TFAA (680 mg, 3.2 mg) and DCM (20 mL). The reaction was stirred for 2 hours at 25° C. The solid were filtered and filtrate was removed under vacuum. The residue was purified through flash column chromatography to provide 200 mg of 168-2 (yield: 36.1%) as an off-white solid. Mass (m/z): 342.8 [M+H]⁺.

Step 2. Preparation of N-(4-(tert-butyl)phenyl)-N-(2,2,2-trifluoroethyl)cyclohexane-1,4-diamine (168)

A solution of 168-2 (200 mg, 0.58 mmol), BH₃-THF (10 ml) in THF (5 ml) was stirred at 70° C. for 18 hours. The solids were filtered and solvent was removed under vacuum. The residue was purified by prep-HPLC (column-Xbridge-C18 150×19 mm, 5 μm; Mobile phase: ACN-H₂O (0.1% FA), 25%-40%) to afford 168A (15.4 mg) as a white solid and 168B (8.3 mg) as a white solid. 168A: ¹H NMR (400 MHZ, CD₃OD) δ 7.15-7.11 (m, 2H), 6.62-6.58 (m, 2H), 3.45-3.37 (m, 1H), 3.20 (q, J=9.8 Hz, 2H), 2.69 (td, J=7.4, 3.6 Hz, 1H), 1.76-1.68 (m, 2H), 1.68-1.60 (m, 4H), 1.59-1.50 (m, 2H), 1.23 (s, 9H). Mass (m/z): 329.3 [M+H]⁺. HPLC: Rt=4.699 min (Column: XBRIDGE 3.5 μm 2.1*50 mm, Mobile phase: H₂O (0.05% TFA)-ACN (0.05% TFA), ACN from 0 to 60% over 7 minutes, Flow rate: 0.8 mL/min). 168B: ¹H NMR (400 MHZ, CD₃OD) δ 7.13 (d, J=8.6 Hz, 2H), 6.59 (d, J=8.6 Hz, 2H), 3.23-3.18 (m, 2H), 3.17 (d, J=3.8 Hz, 1H), 2.52 (dt, J=9.6, 4.8 Hz, 1H), 2.10-1.93 (m, 4H), 1.23 (s, 9H), 1.21-1.10 (m, 4H). Mass (m/z): 329.3 [M+H]⁺. HPLC: Rt=5.096 min (Column: XBRIDGE 3.5 μm 2.1*50 mm, Mobile phase: H₂O (0.05% TFA)-ACN (0.05% TFA), ACN from 0 to 60% over 7 minutes, Flow rate: 0.8 mL/min).

### Compound 169

### 1-N-(4-(tert-butyl)phenyl)-N4-(2-methoxyethyl)cyclohexane-1,4-diamine (169)

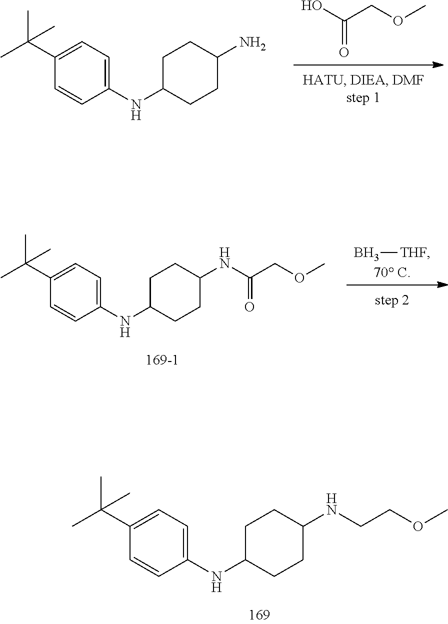

Step 1. Preparation of 1-N-(4-((4-(tert-butyl)phenyl)amino)cyclohexyl)-2-methoxy acetamide (169-1)

A mixture of 1-N-(4-(tert-butyl)phenyl)cyclohexane-1,4-diamine (300 mg, 1.06 mmol), 2-methoxyacetic acid (114 mg, 1.27 mmol), HATU (605 mg, 1.6 mmol), DIEA (685 mg, 5.3 mmol) in DMF (10 mL) was stirred overnight at 25° C. It was quenched by H₂O (30 mL) and extracted with DCM (3×30 mL). The organic layers were combined, washed with brine NaCl (2×30 mL) and dried with MgSO₄, filtered and concentrated. The crude product was applied onto a silica gel column (4 g) eluted with PE: EA (5:1) to give product (100 mg, yield: 90%) as a yellow solid. Mass (m/z): 318.9 [M+H]⁺.

Step 2. Preparation of 1-N-(4-(tert-butyl)phenyl)-N4-(2-methoxyethyl)cyclohexane-1,4-diamine (169)

To a solution of 169-1 (100 mg, 0.31 mmol) in THF (5 mL) was added BH₃-THF (15 mL). Then the mixture was stirred 16 hours at 70° C. The reaction was concentrated under vacuum. The residue was purified by prep-TLC to afford the desired product (10.8 mg) as a white solid. ¹HNMR (400 MHZ, DMSO-d₆) δ 7.04 (d, J=8.4 Hz, 2H), 6.49 (d, J=8.4 Hz, 2H), 3.49 (t, J=5.2 Hz, 2H), 3.32 (d, J=6.6 Hz, 2H), 3.25 (s, 3H), 2.93-2.86 (m, 2H), 1.75-1.45 (m, 8H), 1.16 (d, J=6.8 Hz, 9H). Mass (m/z): 452.3 [M+H]⁺.

### Compound 170

### 1-N-(4-(tetrahydro-2H-pyran-+-yl)phenyl)cyclohexane-1,4-diamine

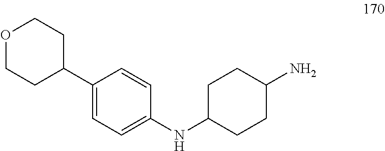 [SMILES: NC1CCC(Nc2ccc(C3CCOCC3)cc2)CC1]

The desired product (52.8 mg) as a white solid was prepared from tert-butyl (4-((4-(tetrahydro-2H-pyran-4-yl)phenyl)amino)cyclohexyl)carbamate (300 mg, 0.8 mmol), 1,4-dioxane (5 mL) and HCl/1,4-dioxane (5 mL; 4N) according to the procedure for 37. ¹H NMR (400 MHZ, DMSO-d₆) δ 6.94 (d, J=8.5 Hz, 2H), 6.55 (d, J=8.4 Hz, 2H), 5.32 (br, 1H), 3.91 (dd, J=10.4, 3.1 Hz, 2H), 3.38 (d, J=3.1 Hz, 2H), 3.00 (br, 1H), 2.52 (br, 1H), 1.76-1.52 (m, 12H). Mass (m/z): 275.3 [M+H]⁺.

### Compound 171

### 4-(4,4-dimethylcyclohexyl)-N-(3-(pyrrolidin-1-yl)propyl)aniline

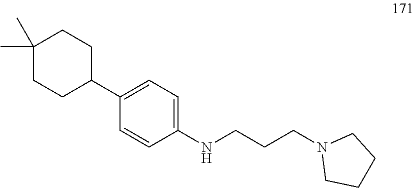 [SMILES: CC1(C)CCC(c2ccc(NCCCN3CCCC3)cc2)CC1]

The desired product 171 (45 mg) as a white solid was prepared from N-(4-(4,4-dimethylcyclohexyl)phenyl)-3-(pyrrolidin-1-yl) propanamide (80 mg, 0.24 mmol), THF (1 mL) and BH₃-THF (30 mL) according to the procedure for 169. ¹H NMR (400 MHZ, DMSO-d₆) δ 7.03 (d, 2H), 6.61 (d, 2H), 3.52 (br, 2H), 3.19 (t, 2H), 3.10 (t, 2H), 3.00 (br, 2H), 2.29-2.24 (m, 1H), 2.05-1.85 (m, 6H), 1.55-1.23 (m, 8H), 0.95 (d, 6H). Mass (m/z): 315.3 [M+H]⁺.

### Compound 172

### 1-methyl-N4-(4-(tert-pentyl)phenyl)cyclohexane-1,4-diamine

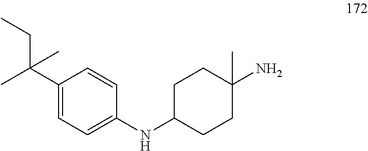 [SMILES: CCC(C)(C)c1ccc(NC2CCC(C)(N)CC2)cc1]

The title compound 172 (96.9 mg) was prepared in a total yield of 91.8% as a white solid from tert-butyl (1-methyl-4-((4-(tert-pentyl)phenyl)amino)cyclohexyl)carbamate (144 mg, 0.385 mmol), TFA (1 mL), and DCM (10 mL) according to the procedure for 24. ¹H NMR (400 MHZ, DMSO-d₆) δ 8.30 (d, J=36.0 Hz, 3H), 7.02 (d, J=8.4 Hz, 2H), 6.56 (d, J=8.4 Hz, 2H), 5.08 (s, 1H), 3.14 (s, 1H), 1.96 (d, J=14.8 Hz, 2H), 1.81-1.61 (m, 4H), 1.52 (q, J=7.6 Hz, 3H), 1.30 (d, J=8.0 Hz, 3H), 1.16 (d, J=1.2 Hz, 6H), 0.61 (t, J=7.2 Hz, 3H). Mass (m/z): 275.3 [M+H]⁺.

### Compound 173

### N1,N1-dibutyl-N2-(4-cyclohexylphenyl)ethane-1,2-diamine

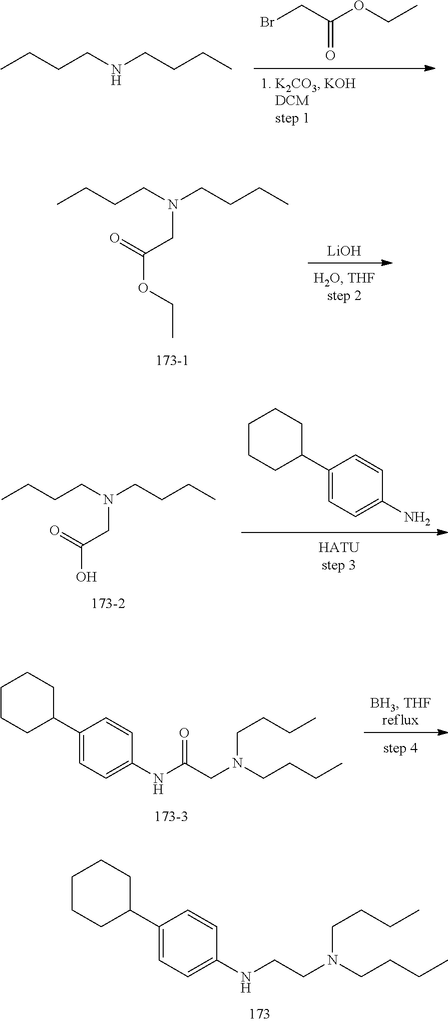

Step 1. Preparation of ethyl dibutylglycinate (173-1)

A mixture of dibutylamine (1 g, 7.74 mmol), ethyl 2-bromoacetate (1.55 g, 9.28 mmol), KOH (434 mg, 7.74 mmol), K₂CO₃ (1.07 g, 7.74 mmol) in DCM (40 mL) was stirred overnight at 40° C. After cooling to rt. 40 mL of water was added. The solid was purified by silica gel chromatography. Target product (1.58 g, 95%) was obtained as colorless oil. Mass (m/z): 216.2 [M+H]⁺.

Step 2. Preparation of dibutylglycine (173-2)

A mixture of ethyl dibutylglycinate (1 g, 4.64 mmol), LiOH (334 mg, 14 mmol) in H₂O (10 mL) and THF (10 mL) was stirred overnight at room temperature. The reaction was concentrated under vacuum. The solid (840 mg, 97%) was used in the next step directly. Mass (m/z): 188.2 [M+H]⁺.

Step 3. Preparation of N-(4-cyclohexylphenyl)-2-(dibutylamino) acetamide (173-3)

A mixture of dibutylglycine (840 mg, 4.49 mmol), 4-cyclohexylaniline (943 mg, 5.38 mmol), HATU (2.05 g, 5.38 mmol), DIPEA (870 mg, 6.37 mmol) in DMF (20 mL) was stirred overnight at room temperature. Then 40 mL of water was added. The solid was purified by silica gel chromatography. Target product (1.6 g, 75%) was obtained as a yellow solid. Mass (m/z): 345.2 [M+H]⁺.

Step 4. Preparation of N1,N1-dibutyl-N2-(4-cyclohexylphenyl)ethane-1,2-diamine (173)

N-(4-cyclohexylphenyl)-2-(dibutylamino) acetamide (200 mg, 0.58 mmol) was added to BH₃-THF (20 mL) and the mixture was stirred overnight at 70° C. The reaction was concentrated under vacuum. The residue was purified by prep-TLC to afford the desired product as a white solid. (20 mg, 10%). ¹H NMR (400 MHZ, CD₃OD) δ 7.03 (d, J=8.5 Hz, 2H), 6.65 (d, J=8.6 Hz, 2H), 3.48 (d, J=6.0 Hz, 2H), 3.35 (d, J=5.9 Hz, 2H), 3.22-3.13 (m, 4H), 2.44-2.33 (m, 1H), 1.86-1.72 (m, 4H), 1.72-1.61 (m, 4H), 1.49-1.33 (m, 8H), 1.33-1.26 (m, 2H), 0.98 (t, J=7.3 Hz, 6H). Mass (m/z): 331.2 [M+H]⁺.

### Compound 174

### N1-(4-propoxyphenyl)cyclohexane-1,4-diamine

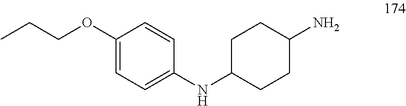 [SMILES: CCCOc1ccc(NC2CCC(N)CC2)cc1]

The desired product 174 as white solid (82.2 mg, 18.8%) was prepared from tert-butyl (4-((4-propoxyphenyl)amino)cyclohexyl)carbamate (600 mg, 11.73 mmol), 1,4-dioxane (10 mL) and 1,4-dioxane/HCl (10 mL) according to the procedure for 37. ¹H NMR (400 MHz, DMSO-d₆) δ 8.49 (s, 1H), 6.69 (d, 2H), 6.57 (d, 2H), 3.77 (t, 2H), 3.37 (br, 1H), 3.02 (br, 1H), 1.83-1.54 (m, 8H), 0.94 (t, 3H). Mass (m/z): 249.3 [M+H]⁺.

### Compound 175

### N-(2-(1H-imidazol-1-yl)ethyl)-4-(tert-butyl)aniline

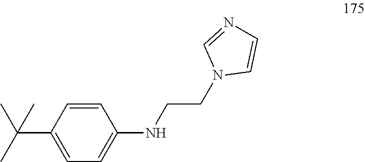 [SMILES: CC(C)(C)c1ccc(NCCn2ccnc2)cc1]

The desired product 175 as white solid (34.8 mg, 11.7%) was prepared from N-(4-(tert-butyl)phenyl)-2-(1H-imidazol-1-yl) acetamide (312 mg, 1.21 mmol), THF (1 mL) and BH₃-THF (30 mL) according to the procedure for 169. ¹H NMR (400 MHz, DMSO-d₆) δ 9.06 (s, 1H), 7.78 (s, 1H), 7.68 (s, 1H), 7.11 (d, 2H), 6.54 (d, 2H), 4.33 (br, 2H), 3.48 (br, 2H), 1.21 (s, 9H). Mass (m/z): 244.3 [M+H]⁺.

### Compound 176

### N1-(4-(4,4-dimethylcyclohexyl)phenyl)ethane-1,2-diamine

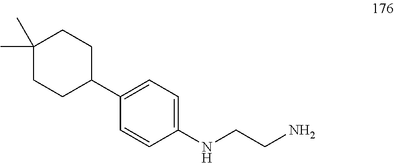 [SMILES: CC1(C)CCC(c2ccc(NCCN)cc2)CC1]

The desired product 176 as a white solid (37.4 mg, 95.3%) was prepared from tert-butyl (2-((4-(4,4-dimethylcyclohexyl)phenyl)amino)ethyl)carbamate (55 mg, 0.16 mmol), 1,4-dioxane (10 mL) and 1,4-dioxane/HCl (10 mL) according to the procedure for 37. ¹H NMR (400 MHZ, DMSO-d₆) δ 6.96 (d, 2H), 6.51 (d, 2H), 3.19 (br, 2H), 2.88 (br, 2H), 2.28-2.20 (m, 1H), 1.54-1.24 (m, 8H), 0.93 (d, 6H). Mass (m/z): 247.3 [M+H]⁺.

### Compound 177

### N-(2-(1H-imidazol-5-yl)ethyl)-4-(tert-butyl)aniline

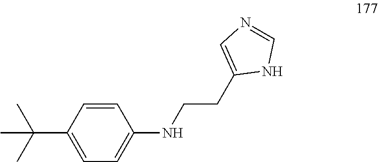 [SMILES: CC(C)(C)c1ccc(NCCc2cnc[nH]2)cc1]

The desired product (65.1 mg, 22.9%) as brown oil was prepared from N-(4-(tert-butyl)phenyl)-2-(1H-imidazol-5-yl) acetamide (300 mg, 1.17 mmol), THF (1 mL) and BH₃-THF (30 mL) according to the procedure for 169. ¹H NMR (400 MHZ, DMSO-d₆) δ 9.06 (s, 1H), 7.78 (s, 1H), 7.68 (s, 1H), 7.11 (d, J=8.6 Hz, 2H), 6.53 (d, J=8.6 Hz, 2H), 4.32 (t, J=5.7 Hz, 2H), 3.47 (t, J=5.7 Hz, 2H), 1.21 (s, 9H). Mass (m/z): 244.3 [M+H]⁺.

### Compound 178

### 4-((4-(tert-butyl)phenyl)amino)piperidine-1-carboxamide

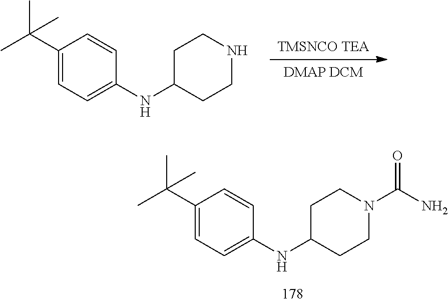

A mixture solution of N-(4-(tert-butyl)phenyl)piperidin-4-amine (100 mg, 0.43 mmol), TMSNCO (50 mg, 0.43 mmol), TEA (87 mg, 0.86 mmol) and DMAP (11 mg, 0.086 mmol) in DCM (5 mL) was stirred at R.T. for 18 hours. The solid was filtered and solvent was removed under vacuum. The residue was purified by prep-HPLC (column-Xbridge-C18 150×19 mm, 5 μm; Mobile phase: ACN-H₂O (0.1% FA), 25%-40%) to afford 178 (38.2 mg) as a white solid. ¹H NMR (400 MHZ, CD₃OD) δ 7.19-7.13 (m, 2H), 6.68-6.61 (m, 2H), 3.95 (s, 2H), 3.45 (tt, J=10.2, 4.0 Hz, 1H), 2.97 (d, J=2.2 Hz, 2H), 2.04-1.94 (m, 2H), 1.42-1.29 (m, 2H), 1.25 (s, 9H). Mass (m/z): 276.2 [M+H]⁺.

### Compound 179

### 2-(2-((4-(tert-butyl)phenyl)amino) ethoxy) ethan-1-ol

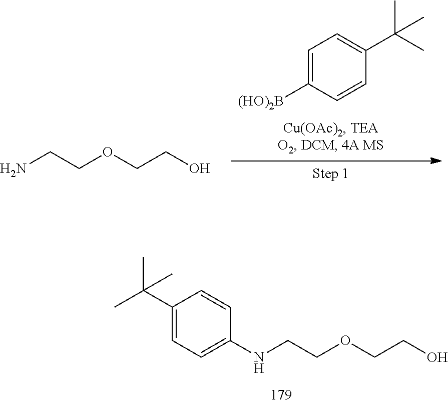

A 100-mL round-bottom flask was charged with 2-(2-aminoethoxy) ethan-1-ol (400 mg, 3.8 mmol, 1.00 eq), (4-(tert-butyl)phenyl) boronic acid (677 mg, 3.8 mmol, 1.00 eq), Cu (OAc) 2 (1382 mg, 7.6 mmol, 2.00 eq) and TEA (1924 mg, 19 mmol, 5.00 eq) and 4 Å MS (1 g). The reaction was stirred at R.T. under O₂ atmosphere for 18 hours. The solid was filtered and solvent was removed under vacuum. The residue was purified by prep-HPLC (column-Xbridge-C_{18 150×19} mm, 5 μm; Mobile phase: ACN-H₂O (0.1% FA), 25%-40%) to afford 179 (40.1 mg) as a white solid. ¹H NMR (400 MHZ, CD₃OD) δ 7.64-7.59 (m, 2H), 7.44-7.39 (m, 2H), 3.76 (dd, 3.9 Hz, 2H), 3.71-3.67 (m, 2H), 3.64-3.60 (m, 2H), 3.58 (d, 2H), 1.34 (d, 9H). Mass (m/z): 238.2 [M+H]⁺.

### Compound 180

### 4-((4-(tert-butyl)phenyl)amino)piperidine-1-sulfonamide

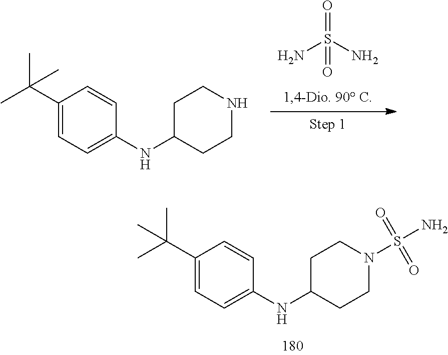

A 10-mL round-bottom flask was charged with N-(4-(tert-butyl)phenyl)piperidin-4-amine (100 mg, 0.43 mmol), 1,4-Dioxane (5 mL) and sulfuric diamide (50 mg, 0.52 mmol). The reaction was stirred for 18 hours at 90° C. The reaction mixture was filtered and the filtrate was concentrated. The residue was purified by prep-HPLC (column-Xbridge-C18 150×19 mm, 5 μm; Mobile phase: ACN-H₂O (0.1% FA), 25%-40%) to afford 180 (21.5 mg) as a white solid. ¹H NMR (400 MHZ, CD₃OD) δ 7.19-7.14 (m, 2H), 6.63 (d, 2H), 3.60 (d, 2H), 3.37-3.32 (m, 1H), 2.78 (td, 2H), 2.08 (d, 2H), 1.55-1.45 (m, 2H), 1.25 (s, 9H). Mass (m/z): 311.9 [M+H]⁺.

### Compound 181

### N1-(4-cyclopropylphenyl)cyclohexane-1,4-diamine

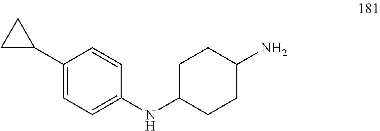 [SMILES: NC1CCC(Nc2ccc(C3CC3)cc2)CC1]

The title compound 181 (47 mg, 55.8%) as a white solid was prepared from tert-butyl (4-((4-cyclopropylphenyl)amino)cyclohexyl)carbamate (130 mg) and HCl in 1,4-dioxane (10 mL, 4 N) according to the procedure for 37. ¹H NMR (400 MHZ, CD₃OD) δ 7.24-6.98 (m, 4H), 3.15-3.07 (m, 2H), 2.20-2.00 (m, 4H), 1.97-1.81 (m, 1H), 1.58-1.35 (m, 4H), 1.02-0.88 (m, 2H), 0.71-0.56 (m, 2H). MS (m/z): 231.2 [M+H]⁺.

### Compound 182

### N1-(3,4,5-trimethylphenyl)cyclohexane-1,4-diamine

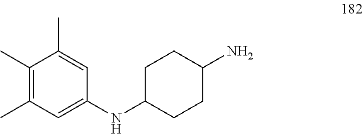 [SMILES: Cc1cc(NC2CCC(N)CC2)cc(C)c1C]

Step 2. Preparation of N1-(3,4,5-trimethylphenyl)cyclohexane-1,4-diamine (182)

The title compounds 182A (47.4 mg) as a white solid and 182B (40.3 mg) as a white solid were prepared from tert-butyl (4-((3,4,5-trimethylphenyl)amino)cyclohexyl)carbamate (110 mg, 0.33 mmol) and HCl in 1,4-dioxane (10 mL, 4 N) according to the procedure for 37. 182A: ¹H NMR (400 MHZ, CD₃OD) § 6.87 (s, 2H), 3.57 (br, 1H), 3.35 (br, 1H) 2.29 (s, 6H), 2.16 (s, 3H), 1.86 (br, 8H). Mass (m/z): 233.3 [M+H]⁺. HPLC: Rt: 3.206 min (Column: XBRIDGE 2.1*50 mm, 3.5 μm; Mobile Phase: H₂O (0.05% TFA)/ACN (0.05% TFA), ACN from 0% to 60% over 7 minutes, 7-8 min, ACN from 60% to 100%; 0.8 mL/min). 182B: ¹H NMR (400 MHz, CD₃OD) δ 6.93 (s, 2H), 3.56 (s, 1H), 3.28 (s, 1H), 2.20 (s, 6H), 2.11 (s, 3H), 1.49 (br, 8H). Mass (m/z): 233.3 [M+H]⁺. HPLC: Rt: 3.685 min (Column: XBRIDGE 2.1*50 mm, 3.5 μm; Mobile Phase: H₂O (0.05% TFA)/ACN (0.05% TFA), ACN from 0% to 60% over 7 minutes, 7-8 min, ACN from 60% to 100%; 0.8 mL/min).

### Compound 183

### N1-(4-(tert-butyl)-2-chlorophenyl)cyclohexane-1,4-diamine

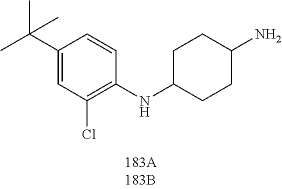 [SMILES: CC(C)(C)c1ccc(NC2CCC(N)CC2)c(Cl)c1]

The title compound 183 was prepared from commercial 4-(tert-butyl)-2-chloroaniline (18.3 mg, 0.1 mmol) according to the procedure for compound 24. The mixture was purified by preparative HPLC (Column: X Select-CSH-Prep 5 μm OBD, 19*150 mm; ACN/water (0.5% TFA)=5%-305%-95%-95%-10%, 0 min-10 min-10.5 min-11.5 min-13 min) to afford compound 183A (Rt=7.49 min) as a yellow solid and compound 184B (Rt=7.26 min) as a yellow solid. 183A (5.4 mg, 19%): ¹H NMR (400 MHZ, DMSO-d₆) 8.05 (s, 2H), 7.26-7.09 (m, 2H), 6.72 (d, J=8.8 Hz, 1H), 4.40 (d, J=7.6 Hz, 1H), 3.59 (s, 1H), 3.25 (s, 1H), 1.82-1.62 (m, 8H), 1.21 (s, 9H). Mass (m/z): 281.4 [M+H]⁺. 183B (18.4 mg, 66%): ¹H NMR (400 MHz, DMSO-d₆) 8.06 (s, 2H), 7.28-7.11 (m, 2H), 6.74 (d, J=8.8 Hz, 1H), 4.55 (d, J=7.6 Hz, 1H), 3.42 (s, 1H), 2.98 (s, 1H), 2.05-1.94 (m, 4H), 1.53-1.31 (m, 4H), 1.21 (s, 9H). Mass (m/z): 281.4 [M+H]⁺.

### Compound 184

### N1-(4-(tert-butyl)-2-methylphenyl)cyclohexane-1,4-diamine

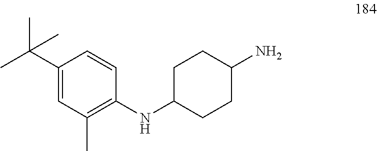 [SMILES: Cc1cc(C(C)(C)C)ccc1NC1CCC(N)CC1]

The title compound 184 (15.6 mg) was prepared in a total yield of 43% as a yellow solid with 1:2 mixture by ¹H NMR from 4-(tert-butyl)-2-methylaniline (16 mg, 0.1 mmol) according to the procedure for compound 24. ¹H NMR (400 MHZ, DMSO-d₆) δ 8.20 (s, 3H), 7.01 (s, 1H), 6.99-6.96 (m, 1H), 6.52-6.45 (m, 1H), 3.53 (s, 1H), 3.20-3.05 (m, 0.6 H), 3.02-2.84 (m, 0.3H), 2.14 (s, 2H), 2.05 (s, 1H), 2.00 (m, 2H), 1.87-1.41 (m, 6H), 1.20 (s, 9H). Mass (m/z): 261.4 [M+H]⁺.

### Compound 185

### N2-(2-methyl-6-(2,2,6-trimethylmorpholino)pyridin-3-yl)spiro[3.3]heptane-2,6-diamine

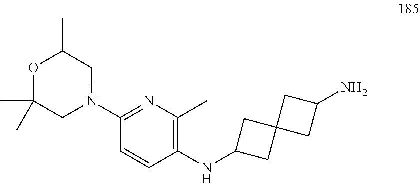 [SMILES: Cc1nc(N2CC(C)OC(C)(C)C2)ccc1NC1CC2(CC(N)C2)C1]

The title compound 185 (23.3 mg) was prepared in a total yield of 67.5% as a yellow solid according to the procedure for compound 24. ¹H NM R (400 MHZ, DMSO-d₆) δ 6.78 (d, J=8.8 Hz, 1H), 6.53 (d, J=8.8 Hz, 1H), 4.02-3.80 (m, 2H), 3.77-3.63 (m, 2H), 3.53 (m, 1H), 2.43-2.31 (m, 2H), 2.27-2.14 (m, 5H), 2.10 (m, 1H), 2.01-1.87 (m, 2H), 1.22 (s, 3H), 1.16 (s, 3H), 1.07 (d, J=6.0 Hz, 3H). Mass (m/z): 345.2 [M+H]⁺

### Compound 186

### N2-(4-(3,5-dimethyl-4-(2,2,2-trifluoroethyl) piperazin-1-yl)-3-fluorophenyl) spiro-[3.3]heptane-2,6-diamine

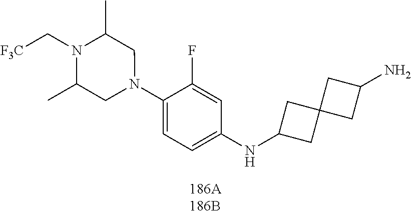 [SMILES: CC1CN(c2ccc(NC3CC4(CC(N)C4)C3)cc2F)CC(C)N1CC(F)(F)F]

The title compounds 186A (5.4 mg) as a white solid and 186B (5.8 mg) as a white solid were prepared according to the procedure for 24. 186A: ¹HNMR (400 MHZ, DMSO-d₆) δ 6.81 (t, J=8.8 Hz, 1H), 6.31-6.26 (m, 2H), 3.76-3.59 (m, 2H), 3.34 (q, J=9.8 Hz, 2H), 3.03 (dd, J=8.6, 2.2 Hz, 2H), 2.93 (d, J=6.2 Hz, 2H), 2.54-2.30 (m, 6H), 2.20-2.09 (m, 2H), 1.94-1.86 (m, 2H), 1.11 (d, J=6.2 Hz, 6H). Mass (m/z): 415.3 [M+H]⁺. HPLC: Rt: 3.919 min (Column: XBRIDGE 2.1*50 mm, 3.5 μm; Mobile Phase: H₂O (0.05% TFA), ACN (0.05% TFA), ACN from 0% to 60% over 7 minutes, 7-8 min, ACN from 60% to 100%; 0.8 mL/min). 186B: ¹H NMR (400 MHZ, DMSO-d₆) δ 6.82 (d, J=8.6 Hz, 1H), 6.32-6.21 (m, 2H), 3.68 (d, J=32.8 Hz, 2H), 3.34 (d, J=10.0 Hz, 2H), 3.09-2.94 (m, 4H), 2.61-2.48 (m, 2H), 2.40 (t, J=10.8 Hz, 4H), 2.14 (s, 2H), 1.96-1.78 (m, 2H), 1.11 (d, J=6.2 Hz, 6H). Mass (m/z): 415.3 [M+H]⁺. HPLC: Rt: 3.921 min (Column: XBRIDGE 2.1*50 mm, 3.5 μm; Mobile Phase: H₂O (0.05% TFA), ACN (0.05% TFA), ACN from 0% to 60% over 7 minutes, 7-8 min, ACN from 60% to 100%; 0.8 mL/min).

### Compound 187

### 2-((4-aminocyclohexyl)amino)-5-(tert-butyl)benzonitrile

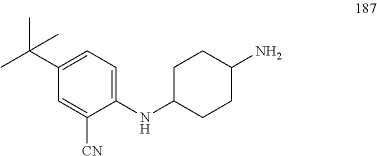 [SMILES: CC(C)(C)c1ccc(NC2CCC(N)CC2)c(C#N)c1]

The title compound 187 (21.9 mg) was prepared in a total yield of 78% as a yellow solid with 1:2 mixture by ¹H NMR from 2-amino-5-(tert-butyl)benzonitrile (17.4 mg, 0.1 mmol) according to the procedure for compound 88. ¹H NMR (400 MHZ, DMSO-d₆) δ 8.16 (s, 3H), 7.28-7.11 (m, 2H), 6.74 (m, 1H), 3.42 (s, 1H), 3.21-3.04 (m, 0.6 H), 3.00-2.88 (m, 0.3H), 2.98 (m, 1H), 2.10-1.93 (m, 4H), 1.56-1.33 (m, 4H), 1.22 (s, 9H). Mass (m/z): 272.3 [M+H]⁺

### Compound 188

### 4-(tert-butyl)-N-(heptan-4-yl)aniline

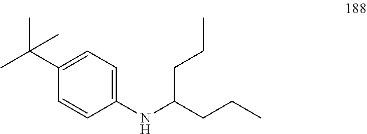 [SMILES: CCCC(CCC)Nc1ccc(C(C)(C)C)cc1]

The title compound 188 (16.9 mg) was prepared in a total yield of 68% as a yellow oil from 4-(tert-butyl)aniline (14.9 mg, 0.1 mmol) according to the procedure for compound 4. ¹H NMR (400 MHz, DMSO-d₆) δ 7.11 (d, J=8.4 Hz, 2H), 6.97 (s, 1H), 6.54 (d, J=8.4 Hz, 2H), 3.11 (m, 1H), 1.54-1.31 (m, 8H), 1.21 (s, 9H), 0.88 (m, 6H). Mass (m/z): 248.3 [M+H]⁺.

### Compound 189

### 1-amino-4-((4-(tert-butyl)phenyl)amino)cyclohexane-1-carboxylic acid

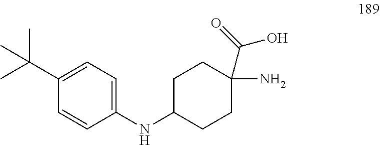 [SMILES: CC(C)(C)c1ccc(NC2CCC(N)(C(=O)O)CC2)cc1]

The title compound 189 (9.1 mg) was prepared in a total yield of 31% as a white solid with 1:1 mixture by ¹H NMR from 4-(tert-butyl)aniline (14.9 mg, 0.1 mmol) according to the procedure for compound 24. ¹H NMR (400 MHZ, DMSO-d₆) δ 7.56 (s, 3H), 7.07 (d, J=8.8 Hz, 1H), 7.04 (d, J=8.8 Hz, 1H), 6.51 (d, J=8.8 Hz, 2H), 3.14 (m, 1H), 2.14-1.81 (m, 3H), 1.80-1.60 (m, 3H), 1.47-1.29 (m, 2H), 1.20 (s, 9H). Mass (m/z): 291.3 [M+H]⁺.

### Compound 190

### N1-(4-(tert-butyl)-2-fluorophenyl)cyclohexane-1,4-diamine

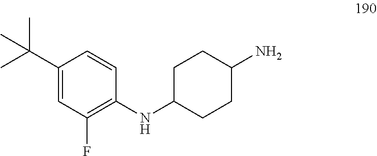 [SMILES: CC(C)(C)c1ccc(NC2CCC(N)CC2)c(F)c1]

The title compound 190 (26.4 mg) was prepared in a total yield of 67% as a white solid with 1:2 mixture by ¹H NMR from 4-(tert-butyl)-2-fluoroaniline (25.1 mg, 0.15 mmol) according to the procedure for compound 88. ¹H NMR (400 MHZ, DMSO-d₆) δ 8.28 (s, 2H), 7.06-6.96 (m, 2H), 6.68 (m, 1H), 4.81 (s, 0.6H), 4.53 (s, 0.3H), 3.13 (m, 1.3H), 2.97-2.85 (m, 0.7H), 1.98 (m, 3H), 1.87-1.56 (m, 3H), 1.47-1.23 (m, 2H), 1.19 (s, 9H). Mass (m/z): 265.2 [M+H]⁺.

### Compound 191

### N1-(2,3-dihydro-1H-inden-4-yl)cyclohexane-1,4-diamine

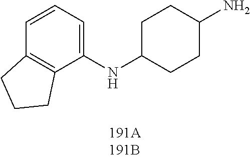 [SMILES: NC1CCC(Nc2cccc3c2CCC3)CC1]

The title compound 191 was prepared tert-butyl from (4-((2,3-dihydro-1H-inden-4-yl)amino)cyclohexyl)carbamate (217 mg, 0.658 mmol), TFA (1 mL), and DCM (10 mL) according to the procedure for 24, which was purified by prep HPLC (solvent system (Column: X Select-CSH-Prep 5 μm OBD, 19*150 mm; ACN/water (0.5% TFA)=15%-25%-95%-95%-10%, 0 min-10 min-10.5 min-11.5 min-13 min) to give the desired products 191A (Rt=8.0 min) as a white solid (47.4 mg, 31.4%) and 191B (Rt=9.2 min) as a white solid (38.2 mg, 25.3%). 191A: ¹H NMR (400 MHZ, DMSO-d₆) δ 6.96 (s, 1H), 6.56 (d, J=44.0 Hz, 2H), 3.19 (s, 1H), 2.95 (s, 1H), 2.77 (t, J=7.6 Hz, 2H), 2.66 (s, 2H), 1.95 (td, J=15.2, 13.2, 5.6 Hz, 6H), 1.45-1.18 (m, 5H). Mass (m/z): 231.3 [M+H]⁺. 191B: ¹H NMR (400 MHz, DMSO-d₆) δ 6.94 (t, J=8.8 Hz, 1H), 6.54 (d, J=57.2 Hz, 2H), 3.50 (d, J=8.8 Hz, 1H), 3.13 (s, 1H), 2.88-2.62 (m, 4H), 1.96 (p, J=7.2 Hz, 2H), 1.83-1.72 (m, 2H), 1.65 (q, J=14.4, 11.6 Hz, 6H). Mass (m/z): 231.3 [M+H]⁺.

### Compound 192

### 4-((4-(tert-butyl)phenyl)amino)cyclohexane-1-carboxamide

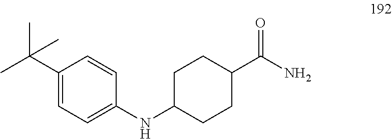 [SMILES: CC(C)(C)c1ccc(NC2CCC(C(N)=O)CC2)cc1]

The title compound 192 (100.3 mg) was prepared in a total yield of 54.5% as a white solid with 1:2 mixture by ¹H NMR with from 4-(tert-butyl)aniline (100 mg, 0.671 mmol), 4-oxocyclohexane-1-carboxamide (142 mg, 1.006 mmol), AcOH (0.1 mL), NaBH (OAc) 3 (286 mg, 1.344 mmol) and DCE (10 mL) according to the procedure for 1-1. ¹H NMR (400 MHZ, DMSO-d₆) δ 7.18 (d, J=12.0 Hz, 1H), 7.03 (dd, J=8.8, 2.8 Hz, 2H), 6.66 (d, J=8.4 Hz, 1H), 6.49 (t, J=9.6 Hz, 2H), 3.36 (s, 1H), 2.17-1.99 (m, 1H), 1.94 (dd, J=12.0, 4.0 Hz, 1H), 1.75 (ddd, J=14.8, 11.6, 5.2 Hz, 2H), 1.63 (d, J=13.2 Hz, 1H), 1.56-1.36 (m, 3H), 1.23 (s, 3H), 1.16 (s, 9H), 1.07 (t, J=12.0 Hz, 1H). Mass (m/z): 275.3 [M+H]⁺.

### Compound 193

### N1-(4-(tert-butyl)phenyl)-N4,N4-dimethylcyclohexane-1,4-diamine

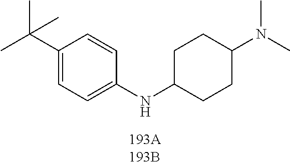 [SMILES: CN(C)C1CCC(Nc2ccc(C(C)(C)C)cc2)CC1]

The title compound 193 was prepared from 4-(tert-butyl)aniline (50 mg, 0.336 mmol), 4-(dimethylamino)cyclohexan-1-one (71 mg, 0.503 mmol), AcOH (0.1 mL), NaBH (OAc) 3 (143 mg, 0.672 mmol) and DCE (10 mL) according to the procedure for 1-1, which was purified by prep HPLC (Column: X Select-CSH-Prep 5 μm OBD, 19*150 mm; ACN/water (0.5% TFA)=15%-25%-75%-85%-11%, 0 min-10 min-10.5 min-11.5 min-13 min), to give the desired product 193A (Rt=7.5 min) as a white solid (28.7 mg, 31.5%) and 193B (Rt=8.8 min) as a white solid (20.2 mg, 21.7%). 193A: ¹H NMR (400 MHz, DMSO-d₆) δ 7.28 (d, J=8.0 Hz, 2H), 6.85 (s, 2H), 3.33-3.08 (m, 2H), 2.73 (d, J=4.8 Hz, 6H), 2.04 (q, J=10.8, 8.8 Hz, 4H), 1.51 (dd, J=13.6, 10.4 Hz, 2H), 1.31 (dd, J=18.8, 7.6 Hz, 2H), 1.24 (s, 9H). Mass (m/z): 275.3 [M+H]⁺. 193B: ¹H NMR (400 MHZ, DMSO-d₆) δ 7.36 (d, J=8.0 Hz, 2H), 7.01 (s, 2H), 3.31 (d, J=22.8 Hz, 1H), 3.24-3.12 (m, 1H), 2.73 (d, J=4.8 Hz, 6H), 2.04 (d, J=11.2 Hz, 4H), 1.61-1.46 (m, 2H), 1.42-1.31 (m, 2H), 1.26 (s, 9H). Mass (m/z): 275.3 [M+H]⁺.

### Compound 194

### N1-(4-(tert-butyl)phenyl)-N3-methylcyclopentane-1,3-diamine

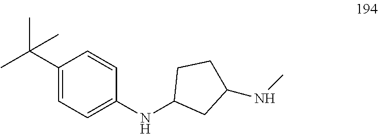 [SMILES: CNC1CCC(Nc2ccc(C(C)(C)C)cc2)C1]

The title compound 194 (8.0 mg) was prepared in a total yield of 13.0% as a brown solid from tert-butyl (3-((4-(tert-butyl)phenyl)amino)cyclopentyl)carbamate (83 mg, 0.25 mmol), LiAlH₄ (37 mg, 1.00 mmol), and THF (5 mL) according to the procedure for 23. ¹H NMR (400 MHZ, DMSO-d₆) δ 7.07-7.04 (m, 2H), 6.51-6.46 (m, 2H), 3.90 (s, 1H), 3.46 (q, J=7.2 Hz, 1H), 2.43 (s, 3H), 2.15-2.03 (m, 3H), 1.88-1.66 (m, 2H), 1.52-1.42 (m, 1H), 1.19 (s, 9H). Mass (m/z): 247.3 [M+H]⁺.

### Compound 195

### N1-(4-(tert-butyl)phenyl)cyclobutane-1,3-diamine

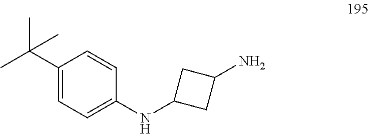 [SMILES: CC(C)(C)c1ccc(NC2CC(N)C2)cc1]

The title compound 195 (12.3 mg) was prepared in a total yield of 15.3% as a yellow oil from tert-butyl (3-((4-(tert-butyl)phenyl)amino)cyclobutyl)carbamate (110 mg, 0.346 mmol), LiAlH₄ (51 mg, 1.384 mmol) and THF (5 mL) according to the procedure for 23. ¹H NMR (400 MHz, DMSO-d₆) δ 6.20 (dd, J=8.8, 2.8 Hz, 2H), 5.60-5.50 (m, 2H), 3.21 (q, J=7.2 Hz, 1H), 2.81-2.67 (m, 1H), 1.78 (dtd, J=9.6, 7.2, 2.8 Hz, 1H), 1.67 (dq, J=6.8, 2.4 Hz, 1H), 1.63 (h, J=2.0 Hz, 2H), 1.53 (d, J=7.8 Hz, 3H), 1.33-1.12 (m, 2H), 0.31 (s, 9H). Mass (m/z): 233.3 [M+H]⁺.

### Compound 196

### 4-(tert-butyl)-N-(4-((dimethylamino)methyl)cyclohexyl)aniline

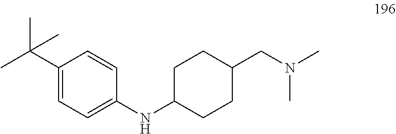 [SMILES: CN(C)CC1CCC(Nc2ccc(C(C)(C)C)cc2)CC1]

The title compound 196 (62.2 mg) was prepared in a total yield of 43.5% as a brown oil from 4-((4-(tert-butyl)phenyl)amino)-N,N-dimethylcyclohexane-1-carboxamide (150 mg, 0.497 mmol), LiAlH₄ (74 mg, 1.987 mmol) and THF (10 mL) according to the procedure for 23. H NMR (400 MHZ, DMSO-d₆) δ 7.07-7.01 (m, 2H), 6.55-6.44 (m, 2H), 5.19 (s, 1H), 3.45-3.33 (m, 2H), 3.05 (s, 1H), 2.61 (d, J=27.2 Hz, 2H), 2.48 (s, 6H), 1.96 (dd, J=13.2, 3.6 Hz, 1H), 1.89-1.82 (m, 1H), 1.64-1.49 (m, 3H), 1.19 (s, 9H). Mass (m/z): 289.3 [M+H]⁺.

### Compound 197

### (1r,4r)-N1-(4-(tert-butyl)phenyl)cyclohexane-1,4-diamine

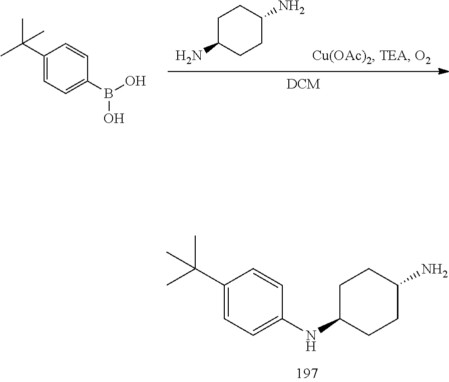

A suspension of 4-(tert-butyl)aniline (178.1 mg, 1.0 mmol), Cu (OAc) 2 (185.4 mg, 1.5 mmol), TEA (0.28 mL, 2.0 mmol) and powdered 4 Å MS (0.75 g) in CH₂Cl₂ (10.0 mL) was stirred for 5 min at room temperature. To this mixture was added (1r,4r)-cyclohexane-1,4-diamine (22.2 mg, 0.2 mmol). The reaction mixture stirred at room temperature under O₂ for 24 h. The reaction mixture was then filtered through a plug of Celite, the resulting solution was extracted with 3×10 mL of ethyl acetate. The organic layers were combined, washed with water (30 mL), dried and concentrated under vacuum. The residue was purified by prep-TLC (MeOH/DCM=1:5) to afford the desired product as a white solid. (70.4 mg, 28.5%). ¹H NMR (400 MHZ, DMSO-d₆) δ 7.28 (d, J=8.4 Hz, 2H), 6.88 (d, J=8.4 Hz, 2H), 3.22 (m, 1H), 2.99 (m, 1H), 2.04-1.92 (m, 4H), 1.45-1.27 (m, 4H), 1.23 (s, 9H). Mass (m/z): 247.2 [M+H]⁺.

### Compound 198

### (1s,4s)-N1-(4-(tert-butyl)phenyl)cyclohexane-1,4-diamine

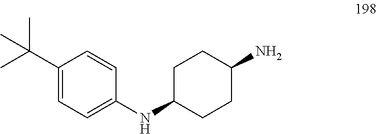 [SMILES: CC(C)(C)c1ccc(N[C@H]2CC[C@@H](N)CC2)cc1]

The title compound 198 (76.1 mg) was prepared in a total yield of 30.8% as a white solid from 4-(tert-butyl)aniline (178.1 mg, 1.0 mmol), (1s,4s)-cyclohexane-1,4-diamine (171 mg, 1.5 mmol) according to the procedure for compound 197. ¹H NMR (400 MHZ, DMSO-d₆) δ7.19 (d, J=8.4 Hz, 2H), 6.71 (d, J=8.4 Hz, 2H), 3.43 (m, 1H), 3.15 (m, 1H), 1.86-1.54 (m, 8H), 1.22 (s, 9H). Mass (m/z): 247.2 [M+H]⁺.

### Compound 199

### N1-(pyridin-4-yl)cyclohexane-1,4-diamine

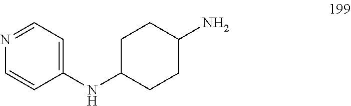 [SMILES: NC1CCC(Nc2ccncc2)CC1]

The title compound 199 (6.1 mg) was prepared in a yield of 1.97% as a white powder with 1:1 mixture by ¹H NMR from pyridin-4-amine (100 mg, 1.06 mmol), tert-butyl (4-oxocyclohexyl)carbamate (339 mg, 1.59 mmol) and according to the procedure for 20. ¹H NMR (400 MHZ, DMSO-d₆) δ 8.15-8.08 (m, 2H), 8.03 (s, 3H), 6.83-6.71 (m, 2H), 3.76 (s, 1H), 2.97 (s, 1H), 2.26 (q, J=2.6, 2.0 Hz, 1H), 2.23 (t, J=2.7 Hz, 1H), 2.14 (dh, J=12.9, 3.3 Hz, 2H), 1.74 (ddd, J=12.7, 11.0, 4.5 Hz, 2H), 1.50-1.37 (m, 2H). Mass (m/z): 192.4 [M+H].

### Compound 200

### N-(4-(2-aminoethyl)cyclohexyl)-4-(tert-butyl)aniline

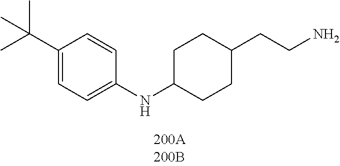 [SMILES: CC(C)(C)c1ccc(NC2CCC(CCN)CC2)cc1]

The title compound 200A (Rt=7.31 min; 53.2 mg) was prepared in a three-step overall yield of 9.8% as a white powder and compound 200B (Rt=8.47 min; 50.2 mg) was prepared in a three-step overall yield of 9.1% as a white powder from 4-(tert-butyl)aniline (300 mg, 2.01 mmol), ethyl 2-(4-oxocyclohexyl) acetate (555 mg, 3.02 mmol) according to the procedure for 84 which were prepared by Prep-HPLC (Column: X Select-CSH-Prep 5 μm OBD, 19*150 mm; ACN/water (0.5% TFA)=5%-33%-95%-95%-5%, 0 min-10.0 min-10.5 min-11.5 min-13.0 min). 200A: ¹H NMR (400 MHZ, DMSO-d₆) δ 7.63 (s, 3H), 7.01-7.32 (br, 2H), 6.41-6.82 (br, 2H), 3.46 (s, 1H), 2.82 (s, 2H), 1.61 (s, 4H), 1.49 (d, J=10.8 Hz, 7H), 1.23 (s, 9H). Mass (m/z): 275.6 [M+H]⁺. 200B: ¹H NMR (400 MHZ, DMSO-d₆) δ 7.59 (s, 3H), 6.94-7.38 (br, 2H), 6.43-6.72 (br, 2H), 2.78 (s, 2H), 1.88-1.97 (m, 2H), 1.73 (d, J=12.4 Hz, 2H), 1.46-1.36 (m, 2H), 1.36-1.23 (br, 2H), 1.22 (s, 9H), 0.98 (d, J=12.8 Hz, 2H). Mass (m/z): 275.6 [M+H]⁺.

### Compound 201

### N1-(4-(tert-butyl)-3-chlorophenyl)cyclohexane-1,4-diamine

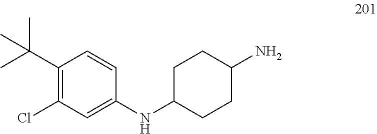 [SMILES: CC(C)(C)c1ccc(NC2CCC(N)CC2)cc1Cl]

The title compound 201 (68.1 mg) was prepared in a yield of 44.54% as a white powder with 5:4 mixture by ¹H NMR from 4-(tert-butyl)-3-chloroaniline (100 mg, 0.54 mmol), tert-butyl (4-oxocyclohexyl)carbamate (174 mg, 0.82 mmol) and according to the procedure for 20. ¹H NMR (400 MHZ, DMSO-d₆) δ 8.18 (s, 2H), 7.13 (dd, J=8.7, 5.7 Hz, 1H), 6.61 (dd, J=20.6, 2.5 Hz, 1H), 6.48 (ddd, J=11.5, 8.7, 2.6 Hz, 1H), 5.64 (dd, J=15.8, 6.9 Hz, 1H), 3.08 (q, J=6.9 Hz, 1H), 3.01-2.89 (m, 1H), 2.04-1.93 (m, 2H), 1.80-1.69 (m, 3H), 1.61 (d, J=8.6 Hz, 1H), 1.54-1.41 (m, 1H), 1.36 (d, J=1.5 Hz, 9H), 1.21-1.10 (m, 1H). Mass (m/z): 281.5 [M+H]⁺.

### Compound 202

### 2-(pyrrolidin-1-yl) acetaldehyde--4-(tert-butyl)-N-(2-(pyrrolidin-1-yl)ethyl)aniline-4-(tert-butyl)aniline

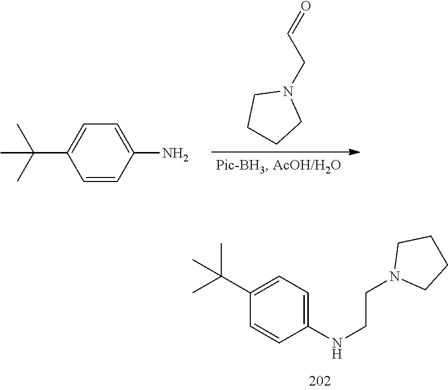

The compound 202 (32 mg, 19%) as a yellow solid was prepared from 4-(tert-butyl)aniline (100 mg, 0.67 mmol), AcOH/H₂O (10:1; 11 mL), 2-(pyrrolidin-1-yl) acetaldehyde (76 mg, 0.67 mmol) and Pic-BH₃ (108 mg, 1.0 mmol) according to the procedure for 90. ¹H NMR (400 MHz, CD₃OD) δ 7.20 (d, J=8.4 Hz, 2H), 6.64 (d, J=8.4 Hz, 2H), 3.47 (t, J=6.0 Hz, 2H), 3.37 (t, J=6.0 Hz, 2H), 3.32 (s, 2H), 3.28 (dt, J=3.2, 1.6 Hz, 2H), 2.06 (br, 4H), 1.26-1.21 (m, 10H). Mass (m/z): 274.2 [M+H]⁺.

### Compound 203

### 1-N-(4-(tert-butyl)phenyl)-N4-(2-methoxyethyl)cyclohexane-1,4-diamine

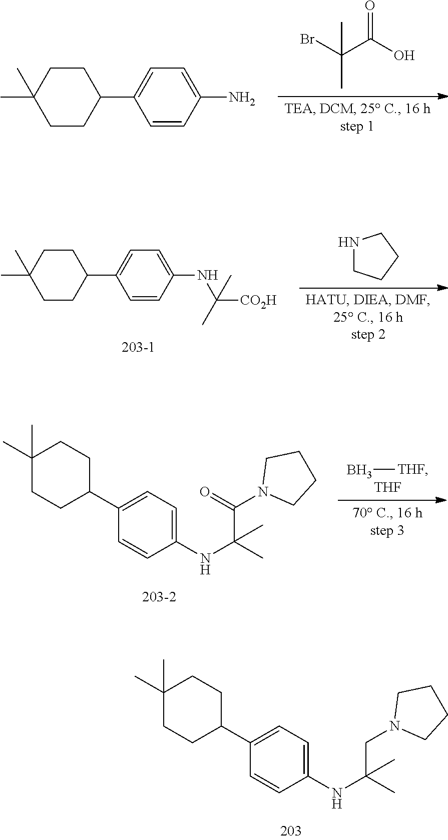

Step 1. 2-((4-(4,4-dimethylcyclohexyl)phenyl)amino)-2-methylpropanoic acid (203-1)

A mixture of 4-(4,4-dimethylcyclohexyl)aniline (100 mg, 0.49 mmol), 2-methoxy-acetic acid (123 mg, 0.73 mmol), TEA (100 mg, 0.98 mmol) in DCM (5 mL) was stirred overnight at 25° C. It was quenched by H₂O (30 mL) and extracted with DCM (3×30 mL). The organic layers were combined, washed with brine NaCl (2×30 mL) and dried with MgSO₄, filtered and concentrated to afford target product as yellow oil. (200 mg, yield: 80%). Mass (m/z): 289.9 [M+H]⁺.

Step 2. 2-((4-(4,4-dimethylcyclohexyl)phenyl)amino)-2-methyl-1-(pyrrolidin-1-yl) propan-1-one (203-2)

To a solution of 203-1 (100 mg, 0.34 mmol), pyrrolidine (29.49 mg, 0.41 mmol) in DMF (5 mL) was added HATU (197 mg, 0.52 mmol) and DIEA (134 mg, 1.03 mmol). Then the mixture was stirred overnight at 25° C. It was quenched by H₂O (30 mL) and extracted with EA (3×30 mL). The organic layers were combined, washed with brine NaCl (2×30 mL) and dried with MgSO₄, filtered and concentrated. The crude product was applied onto a silica gel column (4 g) eluted with PE: EA (10:1) to give product (40 mg, yield: 27%). Mass (m/z): 342.9 [M+H]⁺.

Step 3. Preparation of 4-(4,4-dimethylcyclohexyl)-N-(2-methyl-1-(pyrrolidin-1-yl) propan-2-yl)aniline (203)

To a solution of 203-2 (50 mg, 0.15 mmol), 2-(4-methylpiperazin-1-yl) acetic acid (66 mg, 0.42 mmol) in THF (5 mL) was added BH₃-THF (10 mL). Then the mixture was stirred overnight at rt. The reaction was concentrated under vacuum. The residue was purified by prep-TLC to afford the desired product (15.5 mg) as a white solid. ¹H NMR (400 MHZ, CD₃OD) δ 7.09 (d, J=8.4 Hz, 2H), 6.83 (d, J=8.4 Hz, 2H), 3.67 (br, 2H), 3.49 (br, 2H), 3.30 (br, 2H), 2.38-2.30 (m, 1H), 2.07 (br, 4H), 1.65-1.57 (m, 4H), 1.49 (d, J=11.2 Hz, 2H), 1.39-1.31 (m, 8H), 0.97 (d, J=15.8 Hz, 6H). Mass (m/z): 329.0 [M+H]⁺.

### Compound 204

### 4-N-(4-(tert-butyl)phenyl)-4,5,6,7-tetrahydrobenzo[d]thiazole-2,6-diamine

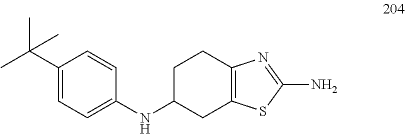 [SMILES: CC(C)(C)c1ccc(NC2CCc3nc(N)sc3C2)cc1]

The title compound 204 (31.6 mg) as a white solid was prepared from 4-(tert-butyl)aniline (90 mg, 0.6 mmol), 2-amino-4,7-dihydrobenzo[d]thiazol-6 (5H)-one (101 mg, 0.6 mmol), Pic-BH₃ (98 mg, 0.9 mmol), H₂O (9 mL) and HOAc (1 mL) according to the procedure for 90. ¹H NMR (400 MHZ, CD₃OD) δ 7.20-7.14 (m, 2H), 6.69-6.64 (m, 2H), 3.77 (dddd, J=10.6, 8.0, 5.0, 2.8 Hz, 1H), 2.92 (dd, J=15.8, 4.4 Hz, 1H), 2.65-2.55 (m, 2H), 2.46-2.39 (m, 1H), 2.18-2.10 (m, 1H), 1.82-1.72 (m, 1H), 1.26 (s, 9H). Mass (m/z): 302.2 [M+H]⁺.

### Compound 205

### 4-((4-(4,4-dimethylcyclohexyl)phenyl)amino)cyclohexan-1-ol

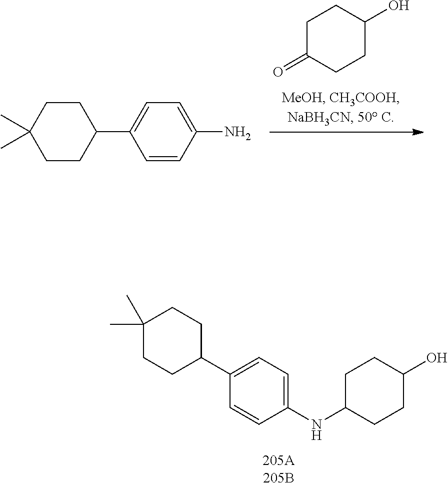 [SMILES: O=C1CCC(O)CC1 ; CC1(C)CCC(c2ccc(N)cc2)CC1 ; CC1(C)CCC(c2ccc(NC3CCC(O)CC3)cc2)CC1]

A mixture of 4-(4,4-dimethylcyclohexyl)aniline (80 mg, 0.39 mmol), 4-hydroxycyclohexan-1-one (53.9 mg, 0.47 mmol) and NaBH₃CN (49.5 mg, 0.79 mmol) in MeOH (10 mL) and CH₃COOH (1 drop) was stirred overnight at 50° C. After cooling, excess MeOH was distilled under vacuum and the residual oil was distilled under vacuum. Then the reaction solution was extracted three times with ethyl acetate (20 mL) and water (20 mL). Then solvent was removed under vacuum and the residue was purified by prep-HPLC (column-Xbridge-C18 150×21.2 mm, 5 μm; Mobile phase: ACN-H₂O (0.1% FA), 32%-37%) to afford the desired products 205A: (30.3 mg, 24.6%) as a white solid and 205B (38.1 mg, 30.9%) as a white solid. 205A: ¹H NMR (400 MHZ, DMSO-d₆) δ 6.93 (d, 2H), 6.50 (d, 2H), 4.54 (br, 1H), 3.39 (br, 1H), 3.08 (br, 1H), 2.26-2.16 (m, 1H), 1.86 (dd, 4H), 1.56-1.46 (m, 4H), 1.42 (d, 2H), 1.31-1.20 (m, 4H), 1.18-1.07 (m, 2H), 0.93 (d, 6H). Mass (m/z): 301.9 [M+H]⁺. HPLC: Rt: 5.674 min (Column: XBRIDGE 2.1*50 mm, 3.5 μm; Mobile Phase: H₂O (0.05% TFA)/ACN (0.05% TFA), ACN from 0% to 60% over 7 minutes, 7-8 min, ACN from 60% to 100%; 0.8 mL/min).

205B: ¹H NMR (400 MHZ, DMSO-d₆) δ 6.90 (d, 2H), 6.49 (d, 2H), 5.16 (br, 1H), 4.37 (br, 1H), 3.68 (br, 1H), 3.18 (br, 1H), 2.20 (br, 1H), 1.58-1.10 (m, 14H), 0.93 (d, 6H). Mass (m/z): 301.9 [M+H]⁺. HPLC: Rt: 5.919 min (Column: XBRIDGE 2.1*50 mm, 3.5 μm; Mobile Phase: H₂O (0.05% TFA)/ACN (0.05% TFA), ACN from 0% to 60% over 7 minutes, 7-8 min, ACN from 60% to 100%; 0.8 mL/min).

### Compound 206

### U4-(tert-butyl)-N-(2-morpholinoethyl)aniline

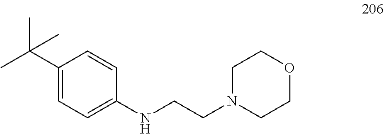 [SMILES: CC(C)(C)c1ccc(NCCN2CCOCC2)cc1]

The desired product 206 (10.1 mg, 7.1%) as a white oil was prepared from 4-tert-butylaniline (80 mg, 0.54 mmol), 2-morpholinoacetaldehyde (83.1 mg, 0.64 mmol), borane-2-picoline complex (86.0 mg, 0.80 mmol), CH₃COOH (1 mL) and H₂O (9 mL) according to the procedure for 90. ¹H NMR (400 MHZ, CD₃OD) δ 7.19-7.14 (m, 2H), 6.64-6.57 (m, 2H), 3.73-3.67 (m, 4H), 3.21 (t, J=6.6 Hz, 2H), 2.59 (t, J=6.6 Hz, 2H), 2.50 (m, 4H), 1.25 (s, 9H). Mass (m/z): 263.0 [M+H]⁺.

### Compound 207

### N-(tert-butyl)-4-(piperidin-4-yl)aniline

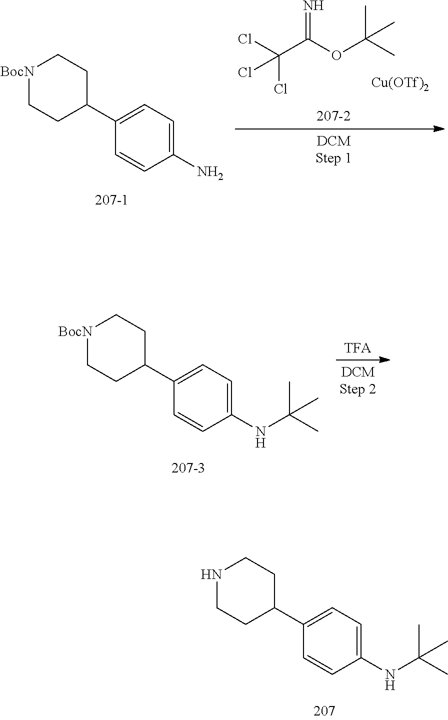 [SMILES: CC(C)(C)OC(=O)N1CCC(c2ccc(N)cc2)CC1 ; CC(C)(C)OC(=N)C(Cl)(Cl)Cl ; CC(C)(C)Nc1ccc(C2CCN(C(=O)OC(C)(C)C)CC2)cc1 ; "Cu(OTf)2" ; CC(C)(C)Nc1ccc(C2CCNCC2)cc1]

Step 1. Preparation of tert-butyl 4-(4-(tert-butylamino)phenyl) piperazine-1-carboxylate (207-3) The title compound 207-3 (33.0 mg) was prepared in a total yield of 10.0% as a white solid from tert-butyl 4-(4-aminophenyl)piperidine-1-carboxylate (276 mg, 1.0 mmol), tert-butyl 2,2,2-trichloroacetimidate (545 mg, 2.5 mmol) and Cu (OTf) 2 (18.1 mg, 50 μmol) according to the procedure for 11. Mass (m/z): 333.3 [M+H]⁺.

Step 2. Preparation of N-(tert-butyl)-4-(piperidin-4-yl)aniline (207) The title compound 207 (6.8 mg) was prepared in a total yield of 30.6% as a yellow solid from tert-butyl 4-(4-(tert-butylamino)phenyl) piperazine-1-carboxylate (33 mg, 0.1 mmol) and TFA (1.0 mL) according to the procedure for 24. ¹H NMR (400 MHZ, DMSO-d₆) δ 10.54 (s, 1H), 8.48 (d, J=83.7 Hz, 1H), 7.35 (s, 4H), 3.36 (d, J=12.5 Hz, 2H), 3.04-2.79 (m, 3H), 1.92 (d, J=13.7 Hz, 2H), 1.73 (qd, J=13.0, 4.0 Hz, 2H), 1.25 (s, 9H). Mass (m/z): 233.3 [M+H]⁺.

### Compound 208

### N1-(6-ethylpyridin-3-yl)cyclohexane-1,4-diamine

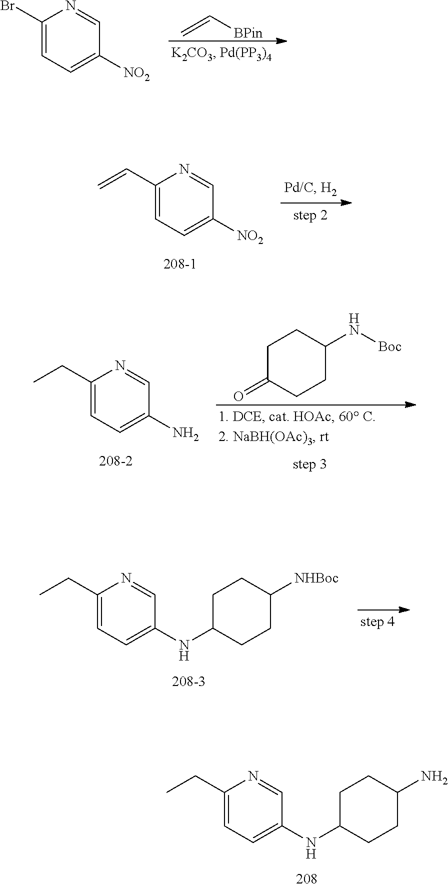

Step 1. Preparation of 5-nitro-2-vinylpyridine (208-1)

To a solution of 2-bromo-5-nitropyridine (500 mg, 2.46 mmol), 4,4,5,5-tetramethyl-2-vinyl-1,3,2-dioxaborolane (569 mg, 3.69 mmol), K₂CO₃ (1019 mg, 7.39 mmol) in 1,4-dioxane (20 mL) and water (2 mL) was added Pd (PPh₃)₄ (285 mg, 0.24 mmol). The mixture was stirred with refluxing overnight under N₂ atmosphere. After cooling to ambient temperature, concentrated under reduced pressure. The residue was purified by silica gel column to provide compound 208-1 (370 mg, 100% yield) as a yellow solid. MS (m/z): 150.9 [M+H]⁺.

Step 2. Preparation of 6-ethylpyridin-3-amine (208-2)

Pd/C (35 mg, 10%) was added to a solution of 2-(4,4-dimethylcyclohex-1-en-1-yl)-5-nitropyridine (350 mg, 2.33 mmol) in MeOH (10 mL), and the mixture was allowed to react under H₂ for 16 h. Then the reaction was filtered and solvent was removed under vacuum to give compound 208-2 (260 mg, 91.2% yield) as a yellow solid. MS (m/z): 123.0 [M+H]⁺.

Step 3. Preparation of tert-butyl (4-((6-ethylpyridin-3-yl)amino)cyclohexyl)carbamate (208-3)

To a solution of compound 208-2 (260 mg, 2.13 mmol) in MeOH (10 mL) and a drop of AcOH was added tert-butyl (4-oxocyclohexyl)carbamate (501 mg, 2.34 mmol). Then the mixture was added NaBH₄ (160 mg, 4.26 mmol) and the mixture was stirred at 25° C. for 16 h. LCMS showed the reaction was completed. The reaction was quenched with water (10 mL), extracted with EA (10 mL*3). The combined organic layers were washed with brine (20 mL), dried over sodium sulfate, filtered and concentrated. The residue was purified by combi-flash with EA/PE (1:3) to afford to give compound 208-3 (0.4 g, 58.8% yield) as a yellow solid. MS (m/z): 319.9 [M+H]⁺.

Step 4. Preparation of N1-(6-ethylpyridin-3-yl)cyclohexane-1,4-diamine (208)

Compound 208-3 (200 mg, 0.63 mmol) and HCl in 1,4-dioxane (10 mL, 4N) were placed in a flask stirred at 25° C. for 16 hrs. Excess 1,4-dioxane was distilled under vacuum and the residue was purified by prep-HPLC (column-Xbridge-C18 150×19 mm, 5 μm; Mobile phase: ACN-H₂O (0.1% FA), 20%-40%) to afford 208 (7.7 mg 5.61%) as a white solid. ¹H NMR (400 MHz, DMSO-d₆) δ 7.92 (d, J=2.5 Hz, 1H), 7.83 (s, 2H), 7.53 (s, 2H), 6.58 (s, 1H), 3.30-3.26 (m, 1H), 3.04 (s, 1H), 2.77 (dd, J=15.2, 7.3 Hz, 2H), 2.52 (d, J=1.9 Hz, 2H), 2.10-1.86 (m, 4H), 1.44 (d, J=11.8 Hz, 2H), 1.21 (t, J=7.6 Hz, 3H). MS (m/z) 220.2 [M+H]⁺.

### Compound 209

### 1-N-(4-(4,4-dimethylcyclohexyl)phenyl)propane-1,3-diamine

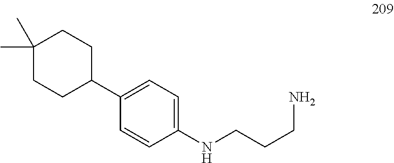 [SMILES: CC1(C)CCC(c2ccc(NCCCN)cc2)CC1]

The title compound 209 (19.8 mg) as a white solid was prepared from tert-butyl (3-((4-(4,4-dimethylcyclohexyl)phenyl)amino)propyl)carbamate (50 mg, 0.14 mmol), 1,4-dioxane (5 mL) and HCl/1,4-dioxane (5 mL) according to the procedure for 24. ¹H NMR (400 MHZ, CD₃OD) δ 7.17 (d, J=8.2 Hz, 2H), 6.89 (d, J=8.4 Hz, 2H), 3.06-3.02 (m, 2H), 2.41-2.34 (m, 1H), 2.01-1.96 (m, 2H), 1.65-1.35 (m, 10H), 0.99 (s, 3H), 0.95 (s, 3H). Mass (m/z): 260.9 [M+H]⁺.

### Compound 210

### N-[4-(aminomethyl)cyclohexyl]-2, 4,6-trimethylaniline

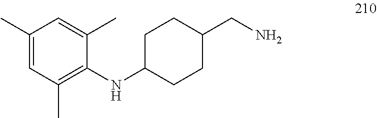 [SMILES: Cc1cc(C)c(NC2CCC(CN)CC2)c(C)c1]

The title compound 210 (16.2 mg) as a yellow oil was prepared from tert-butyl N-({4-[(2,4,6-trimethylphenyl)amino]cyclohexyl}methyl)carbamate (160 mg, 0.46 mmol) and HCl in MeOH (10 mL, 4N) according to the procedure for 37. ¹H NMR (300 MHZ, CD₃OD) δ 6.72 (s, 2H), 3.11 (br, 1H), 2.64 (br, 2H), 2.13 (s, 6H), 2.12 (br, 3H), 1.55 (br, 9H). MS (m/z) 247.0 [M+H]⁺.

### Compound 211

### N1-(4-(tert-butyl)phenyl)-N2-(2-methoxyethyl)-N2-methylethane-1,2-diamine

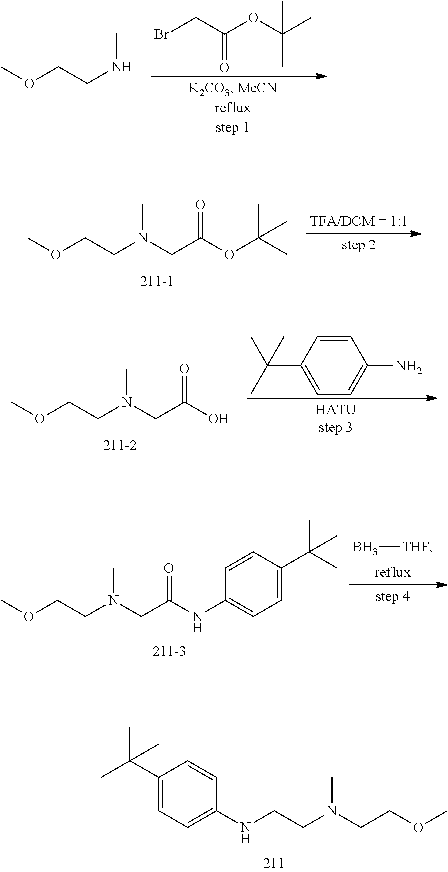

Step 1. Preparation of tert-butyl N-(2-methoxyethyl)-N-methylglycinate (211-1)

A mixture of 2-methoxy-N-methylethan-1-amine (800 mg, 9.00 mmol), tert-butyl 2-bromoacetate (2.45 mg, 12.6 mmol), K₂CO₃ (2.48 g, 18 mmol) in MeCN (50 mL) was stirred overnight at 80° C. After cooling to rt., 40 mL of water was added. The solid was purified by silica gel chromatography. Target product was obtained as colorless oil. Mass (m/z): 204.0 [M+H]⁺.

Step 2. Preparation of N-(2-methoxyethyl)-N-methylglycine (211-2)

A mixture of tert-butyl N-(2-methoxyethyl)-N-methylglycinate (572 mg, 2.81 mmol) in TFA (20 mL) and DCM (2 mL) was stirred overnight at room temperature. The reaction was concentrated under vacuum. The solid was used in the next step directly. Mass (m/z): 148.2 [M+H]⁺.

Step 3. Preparation of N-(4-(tert-butyl)phenyl)-2-((2-methoxyethyl)(methyl)amino) acetamide (211-3)

A mixture of N-(2-methoxyethyl)-N-methylglycine (402 mg, 273 mmol), 4-(tert-butyl)aniline (489 mg, 3.28 mmol), HATU (1.25 g, 3.28 mmol), DIPEA (530 mg, 4.1 mmol) in DMF (20 mL) was stirred overnight at room temperature. Then 40 ml of water was added. The solid was purified by silica gel chromatography. Target product (543 mg, 71%) was obtained as a yellow solid. Mass (m/z): 279.2 [M+H]⁺.

Step 4. Preparation of N1-(4-(tert-butyl)phenyl)-N2-(2-methoxyethyl)-N2-methylethane-1,2-diamine (211)

N-(4-(tert-butyl)phenyl)-2-((2-methoxyethyl)(methyl)amino) acetamide (200 mg, 0.72 mmol) was added to BH₃-THF (20 mL) and the mixture was stirred overnight at 70° C. The reaction was concentrated under vacuum. The residue was purified by prep-TLC to afford the desired product (11 mg, 6%) as a white solid. ¹H NMR (400 MHZ, CD₃OD) δ 7.21 (d, J=8.8 Hz, 2H), 6.66 (d, J=8.7 Hz, 2H), 3.71-3.63 (m, 2H), 3.47 (t, J=6.1 Hz, 2H), 3.33 (s, 3H), 3.28 (d, J=4.9 Hz, 4H), 2.85 (s, 3H), 1.26 (s, 9H). Mass (m/z): 365.2 [M+H]⁺.

### Compound 212

### 1-amino-4-((4-(tert-butyl)phenyl)amino)cyclohexane-1-carboxamide

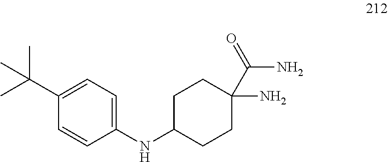 [SMILES: CC(C)(C)c1ccc(NC2CCC(N)(C(N)=O)CC2)cc1]

The title compound 212 (8.0 mg) was prepared in a total yield of 57% as a white solid with 1:1 mixture by ¹H NMR from 1-amino-4-((4-(tert-butyl)phenyl)amino)cyclohexane-1-carboxylic acid (14.5 mg, 0.05 mmol) according to the procedure for compound 24. ¹H NMR (400 MHZ, DMSO-d₆) δ 8.20 (s, 3H), 7.95 (s, 1H), 7.67 (s, 1H), 7.11 (d, J=8.4 Hz, 2H), 6.52 (d, J=8.4 Hz, 2H), 3.28 (m, 0.5H), 2.67 (m, 0.5H), 2.23-2.15 (m, 2H), 2.03-1.86 (m, 4H), 1.63-1.45 (m, 2H), 1.21 (s, 9H). Mass (m/z): 290.1 [M+H]⁺.

### Compound 213

### (1-amino-4-((4-(tert-butyl)phenyl)amino)cyclohexyl)methanol

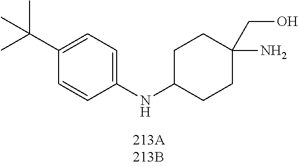 [SMILES: CC(C)(C)c1ccc(NC2CCC(N)(CO)CC2)cc1]

The from title compound 213 1-amino-4-((4-(tert-butyl)phenyl)amino)cyclohexane-1-carboxylic acid (14.5 mg, 0.05 mmol) according to the procedure for compound 86. The mixture was purified by preparative HPLC (Column: X Select-CSH-Prep 5 μm OBD, 19*150 mm; ACN/water (0.5% TFA)=5%-30%-95%-95%-5%, 0 min-10 min-10.5 min-11.5 min-13 min) to afford compound 213A (Rt=3.16 min) as a white solid and compound 213B (Rt=3.57 min) as a white solid. 213A (4.6 mg, 17%): ¹H NMR (400 MHZ, DMSO-d₆) δ 7.85 (s, 2H), 7.36 (s, 1H), 7.17 (d, J=8.4 Hz, 2H), 6.61 (d, J=8.4 Hz, 2H), 3.43 (s, 2H), 2.64 (m, 1H), 1.95-1.51 (m, 4H), 1.22 (s, 9H). Mass (m/z): 277.3 [M+H]⁺. 213B (6.8 mg, 25%): ¹H NMR (400 MHZ, DMSO-d₆) δ 7.83 (s, 2H), 7.34 (s, 1H), 7.08 (d, J=8.4 Hz, 2H), 6.50 (d, J=8.4 Hz, 2H), 3.55 (s, 2H), 2.66 (m, 1H), 2.05-1.87 (m, 4H), 1.63-1.41 (m, 4H), 1.22 (s, 9H). Mass (m/z): 277.3 [M+H]⁺.

### Compound 214

### (2S, 3R)-2-amino-N-(4-((4-(tert-butyl)phenyl)amino)cyclohexyl)-3-hydroxybutanamide

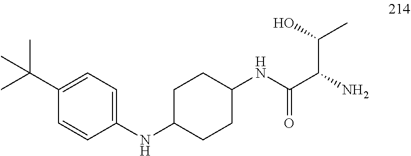 [SMILES: C[C@@H](O)[C@H](N)C(=O)NC1CCC(Nc2ccc(C(C)(C)C)cc2)CC1]

The title compound 214 (11.6 mg) was prepared in a total yield of 68% as a white solid with 1:1 mixture NMR by 1H from tert-butyl ((2S,3R)-1-((4-((4-(tert-butyl)phenyl)amino)cyclohexyl)amino)-3-hydroxy-1-oxobutan-2-yl)ca rbamate (22.4 mg, 0.05 mmol) according to the procedure for compound 1. ¹H NMR (400 MHz, DMSO-d₆) δ 8.18 (s, 1H), 7.05 (d, J=8.4 Hz, 1H), 7.02 (d, J=8.4 Hz, 1H), 6.65 (s, 3H), 6.49 (d, J=8.4 Hz, 1H), 6.45 (d, J=8.4 Hz, 1H), 5.19 (s, 2H), 4.06-3.43 (m, 2H), 3.37 (s, 1H), 3.27 (s, 1H), 1.94 (m, 1H), 1.78 (m, 1H), 1.58 (m, 4H), 1.31 (m, 2H), 1.16 (s, 9H), 1.08 (d, J=3.2 Hz, 1.5H), 1.06 (d, J=3.2 Hz, 1.5H). Mass (m/z): 348.2 [M+H]⁺.

### Compound 215

### N-(4-((4-(tert-butyl)phenyl)amino)cyclohexyl) piperazine-2-carboxamide

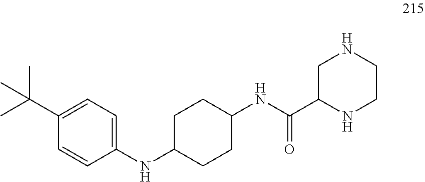 [SMILES: CC(C)(C)c1ccc(NC2CCC(NC(=O)C3CNCCN3)CC2)cc1]

The title compound 215 (7.8 mg) was prepared in a total yield of 43% as a white solid with 1:1 mixture by ¹H NMR from tert-butyl di-tert-butyl 2-((4-((4-(tert-butyl)phenyl)amino)cyclohexyl)carbamoyl) piperazine-1,4-dicarboxylate (27.9 mg, 0.05 mmol) according to the procedure for compound 1. ¹H NMR (400 MHZ, DMSO-d₆) δ 7.55 (s, 1H), 7.06 (d, J=8.4 Hz, 1H), 7.04 (d, J=8.4 Hz, 1H), 6.51 (d, J=8.4 Hz, 1H), 6.47 (d, J=8.4 Hz, 1H), 5.16 (s, 1H), 3.68 (m, 0.5H), 3.50 (m, 0.5H), 3.31 (s, 1H), 3.10 (m, 2H), 2.92-2.53 (m, 4H), 2.46 (s, 1H), 1.95 (m, 1H), 1.76 (m, 1H), 1.65-1.50 (m, 4H), 1.27 (m, 2H), 1.19 (s, 9H). Mass (m/z): 359.3 [M+H]⁺.

### Compound 216

### N-(4-((4-(tert-butyl)phenyl)amino)cyclohexyl)acetamide

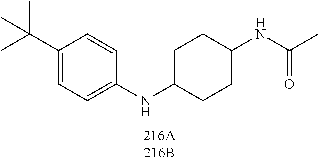 [SMILES: CC(=O)NC1CCC(Nc2ccc(C(C)(C)C)cc2)CC1]

The title compound 216A (Rt=4.57 min; 3 mg) was prepared in a yield of 7.0% as a white powder compound 216B (Rt=4.72 min; 18.2 mg) was prepared in a yield of 18.2% as a white powder from N1-(4-(tert-butyl)phenyl)cyclohexane-1,4-diamine (100 mg, 0.41 mmol), acetic acid (49 mg, 0.81 mmol) according to the procedure for 1 which were prepared by Prep-HPLC (Column: X Select-CSH-Prep 5 μm OBD, 19*150 mm; ACN/water (0.5% TFA)=5%-5%-95%-95%-5%, 0 min-1 min-10 min-11 min-15.0 min). 216A: ¹H NMR (400 MHZ, DMSO-d₆) δ 7.80 (d, J=7.6 Hz, 1H), 7.40 (s, 2H), 7.08 (s, 2H), 3.56-3.42 (m, 1H), 3.31 (s, 1H), 1.95-1.80 (m, 4H), 1.77 (s, 3H), 1.40-1.32 (m, 2H), 1.27 (s, 9H), 1.22-1.16 (m, 2H). Mass (m/z): 289.2 [M+H]⁺. 216B: ¹H NMR (400 MHZ, DMSO-d₆) δ 7.73 (d, J=6.1 Hz, 1H), 7.39 (s, 2H), 7.07 (s, 2H), 3.67 (d, J=5.4 Hz, 1H), 3.38 (dd, J=9.2, 3.7 Hz, 1H), 1.81 (s, 3H), 1.73 (t, J=9.6 Hz, 2H), 1.65 (q, J=6.4, 5.5 Hz, 4H), 1.54-1.42 (m, 2H), 1.25 (s, 9H). Mass (m/z): 289.2 [M+H]⁺.

### Compound 217

### N1-(4-(tert-butyl)-3-methylphenyl)cyclohexane-1,4-diamine

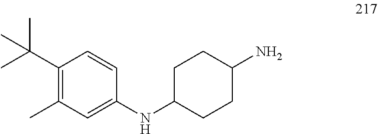 [SMILES: Cc1cc(NC2CCC(N)CC2)ccc1C(C)(C)C]

The title compound 217 (47.9 mg) was prepared in a yield of 30.0% as a white powder with 1:1 mixture by ¹H NMR from 4-(tert-butyl)-3-methylaniline (110 mg, 0.67 mmol), tert-butyl (4-oxocyclohexyl)carbamate (215 mg, 1.01 mmol) and according to the procedure for 20. ¹H NMR (400 MHZ, DMSO-d₆) δ 7.79 (s, 3H), 7.08 (d, J=8.5 Hz, 1H), 6.50 (s, 2H), 3.41 (s, 1H), 3.13 (s, 1H), 2.38 (s, 3H), 1.82-1.65 (m, 6H), 1.62 (d, J=10.1 Hz, 2H), 1.29 (s, 9H). Mass (m/z): 261.3 [M+H]⁺.

### Compound 218

### 5-((4-aminocyclohexyl)amino)-2-(tert-butyl)benzonitrile

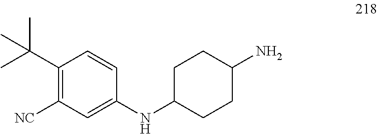 [SMILES: CC(C)(C)c1ccc(NC2CCC(N)CC2)cc1C#N]

The title compound 218 (96.2 mg) was prepared in a yield of 61.7% as a white powder with 1:0.7 mixture by ¹H NMR from 5-amino-2-(tert-butyl)benzonitrile (100 mg, 0.57 mmol), tert-butyl (4-oxocyclohexyl)carbamate (183 mg, 0.86 mmol) and according to the procedure for 20. ¹H NMR (400 MHZ, DMSO-d₆) δ 8.23 (s, 5H), 7.21 (t, J=8.5 Hz, 2H), 6.94 (d, J=2.7 Hz, 1H), 6.91 (d, J=2.6 Hz, 1H), 6.83 (dd, J=8.9, 2.7 Hz, 1H), 6.79 (dd, J=8.8, 2.7 Hz, 1H), 5.92 (d, J=5.9 Hz, 1H), 5.87 (d, J=8.0 Hz, 1H), 3.42 (s, 1H), 3.20-3.01 (m, 2H), 2.94 (ddt, J=11.7, 7.5, 3.6 Hz, 1H), 2.03-1.92 (m, 4H), 1.74 (dq, J=8.8, 5.7 Hz, 5H), 1.67-1.56 (m, 2H), 1.54-1.42 (m, 2H), 1.37 (d, J=1.6 Hz, 16H), 1.27-1.08 (m, 4H). Mass (m/z): 272.7 [M+H]⁺.

### Compound 219

### N1-mesitylcyclohexane-1,4-diamine

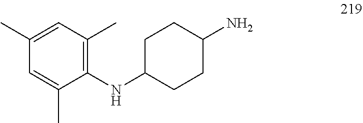 [SMILES: Cc1cc(C)c(NC2CCC(N)CC2)c(C)c1]

The title compound 219 (23.7 mg) was prepared in a total yield of 35.7% as a white solid with 1:2 mixture by ¹H NMR from tert-butyl (4-(mesitylamino)cyclohexyl)carbamate (95 mg, 0.286 mmol), TFA (1 mL), and DCM (10 mL) according to the procedure for 24. ¹H NMR (400 MHZ, DMSO-d₆) δ 6.69 (s, 2H), 3.04 (d, J=39.6 Hz, 1H), 2.91-2.59 (m, 1H), 2.16-2.08 (m, 9H), 1.95-1.89 (m, 1H), 1.77 (d, J=9.2 Hz, 2H), 1.64 (dt, J=10.0, 6.4 Hz, 2H), 1.49 (td, J=9.6, 4.4 Hz, 1H), 1.25 (dt, J=23.2, 11.2 Hz, 2H). Mass (m/z): 233.3 [M+H]⁺.

### Compound 220

### N1-(4′-(tert-butyl)-[1,l′-biphenyl]-4-yl)cyclohexane-1,4-diamine

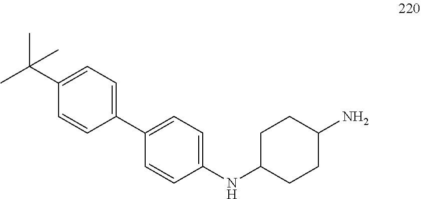 [SMILES: CC(C)(C)c1ccc(-c2ccc(NC3CCC(N)CC3)cc2)cc1]

The title compound 220 (72.6 mg) was prepared in a total yield of 35.7% as a white solid with 1:2 mixture by ¹H NMR from tert-butyl (4-(mesitylamino)cyclohexyl)carbamate (149 mg, 0.353 mmol), TFA (1 mL), and DCM (10 mL) according to the procedure for 24. ¹H NMR (400 MHZ, DMSO-d₆) δ 7.40 (dq, J=8.8, 2.4 Hz, 2H), 7.35-7.31 (m, 4H), 6.68-6.57 (m, 2H), 5.60 (s, 1H), 3.46-2.84 (m, 3H), 2.06-1.68 (m, 5H), 1.64-1.40 (m, 2H), 1.24 (s, 9H). Mass (m/z): 323.3 [M+H]⁺.

### Compound 221

### N1-(naphthalen-2-yl)cyclohexane-1,4-diamine

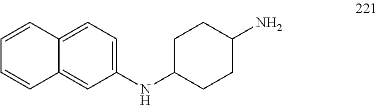 [SMILES: NC1CCC(Nc2ccc3ccccc3c2)CC1]

The title compound 221 (87.2 mg) was prepared in a total yield of 70.7% as a white solid with 1:2 mixture by ¹H NMR from tert-butyl (4-(naphthalen-2-ylamino)cyclohexyl)carbamate (174 mg, 0.512 mmol), TFA (1 mL), and DCM (10 mL) according to the procedure for 24. ¹H NMR (400 MHZ, DMSO-d₆) δ 7.61-7.47 (m, 3H), 7.24 (dddd, J=8.0, 6.8, 3.2, 1.2 Hz, 1H), 7.09-6.86 (m, 2H), 6.68 (dd, J=12.4, 2.4 Hz, 1H), 5.80-5.74 (m, 1H), 3.56-3.17 (m, 3H), 3.12-2.92 (m, 1H), 2.12-2.03 (m, 1H), 2.03-1.95 (m, 1H), 1.85 (dd, J=12.4, 4.0 Hz, 1H), 1.74 (q, J=5.6 Hz, 2H), 1.69-1.59 (m, 1H), 1.56-1.43 (m, 1H). Mass (m/z): 241.3 [M+H]⁺.

### Compound 222

### 4-(2-{4-[(4-tert-butylphenyl)amino]piperidin-1-yl}-2-oxoethyl)-1-methylpiperazin-2-one

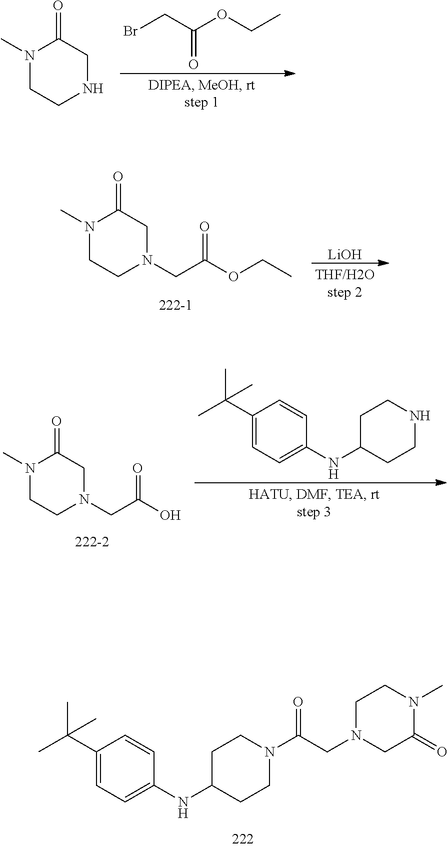

Step 1. Preparation of ethyl 2-(4-methyl-3-oxopiperazin-1-yl) acetate (222-1)

To a solution of 1-methylpiperazin-2-one (400 mg, 3.50 mmol), ethyl 2-bromoacetate (878 mg, 5.25 mmol) and N,N-diisopropylethylamine (906 mg, 7.00 mmol) in MeOH (30 mL) was stirred overnight at 25° C. LCMS showed the reaction was completed. The reaction was quenched with water (50 mL), extracted with EA (50 mL*3). The combined organic layers were washed with brine (20 mL), dried over sodium sulfate, filtered and concentrated. The residue was purified by combi-flash with EA/PE (1:3) to afford to give compound 222-1 (550 mg, 76.8% yield) as a yellow solid. MS (m/z): 200.9 [M+H]⁺.

Step 2. Preparation of (4-methyl-3-oxopiperazin-1-yl) acetic acid (222-2)

A solution of product 222-1 (200 mg, 0.99 mmol) and LiOH (101 mg, 19.9 mmol) in THE/H₂O (1:1) (30 mL) was stirred overnight at 25° C. LCMS showed the reaction was completed. The reaction was concentrated and freeze-drying to afford compound 222-2 (50 mg, 85.47% yield) as yellow oil. Mass (m/z): 173.2 [M+H]⁺.

Step 3. Preparation of 4-(2-{4-[(4-tert-butylphenyl)amino]piperidin-1-yl}-2-oxoethyl)-1-methylpiperazin-2-one (222)

A solution of compound 222-2 (100 mg, 0.58 mmol), N-(4-tert-butylphenyl)piperidin-4-amine (135 mg, 0.58 mmol), HATU (331 mg, 0.87 mmol) and triethylamine (117 mg, 1.16 mmol) in DMF (30 mL) was stirred overnight at 25° C. LCMS showed the reaction was completed. The reaction was quenched with water (50 mL), extracted with EA (50 mL*3). The combined organic layers were washed with brine (40 mL), dried over sodium sulfate, filtered and concentrated. The residue was purified by combi-flash with EA/PE (2:3) to afford to give compound 222 (50 mg, 21.1%) as a white solid. ¹H NMR (400 MHZ, CD₃OD) δ 7.58 (d, J=8.4 Hz, 2H), 7.30 (d, J=8.4 Hz, 2H), 4.59 (br, 1h), 4.12 (br, 1H), 3.84 (s, 3H), 3.78-3.62 (m, 2H), 3.49 (s, 3H), 3.31-3.13 (m, 2H), 3.02 (s, 3H), 2.06 (d, J=11.6 Hz, 2H), 1.86-1.47 (m, 3H), 1.31 (s, 12H). Mass (m/z): 386.9 [M+H]⁺.

### Compound 223

### N1-(4-(2,2,2-trifluoroethyl)phenyl)cyclohexane-1,4-diamine

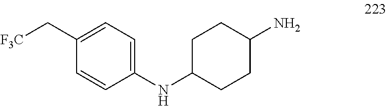 [SMILES: NC1CCC(Nc2ccc(CC(F)(F)F)cc2)CC1]

The desired product 223 (50.8 mg, 24.5%) as white solid was prepared from tert-butyl (4-((4-(2,2,2-trifluoroethyl)phenyl)amino)cyclohexyl)carbamate (278 mg, 0.74 mmol), 1,4-dioxane (10 mL) and 1,4-dioxane/HCl (10 mL) according to the procedure for 37. ¹H NMR (400 MHZ, DMSO-d₆) δ 7.92 (s, 3H), 7.05 (d, 2H), 6.62 (d, 2H), 3.44-3.36 (m, 3H), 3.13 (br, 1H), 1.89-1.39 (m, 8H). Mass (m/z): 273.1 [M+H]⁺.

### Compound 224

### N-(2-(2-aminoethoxy)ethyl)-4-(tert-butyl)aniline

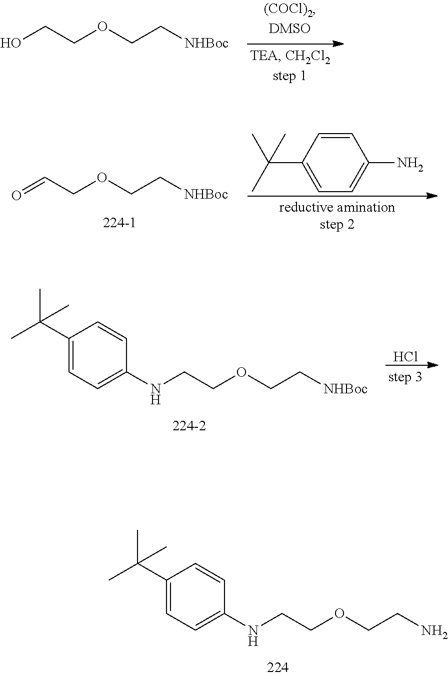 [SMILES: CC(C)(C)OC(=O)NCCOCCO ; CC(C)(C)OC(=O)NCCOCC=O ; CC(C)(C)c1ccc(N)cc1 ; CC(C)(C)OC(=O)NCCOCCNc1ccc(C(C)(C)C)cc1 ; Cl ; CC(C)(C)c1ccc(NCCOCCN)cc1]

Step 1. Preparation of tert-butyl (2-(2-oxoethoxy)ethyl)carbamate (224-1)

A mixture of (COCI) 2 (2.13 g, 16.8 mmol) in CH₂Cl₂ (15 mL) was added dropwise DMSO (2.85 g, 36.5 mmol) in CH₂Cl₂ (15 mL) at −78° C. under nitrogen. After the addition, the solution was stirred for min and a solution of 30 tert-butyl (2-(2-hydroxyethoxy)ethyl)carbamate (1.5 g, 7.31 mmol) in CH₂Cl₂ (15 mL) was then added. After the addition of TEA (6.66 g, 65.8 mmol), the reaction was stirred for 30 min at −78° C. and then was stirred for overnight at room temperature. 100 ml of water was added. The organic phases were combined, dried over Na₂SO₄, filtered and concentrated to afford the product. Target product (1.3 g, 88%) was obtained as a yellow solid. Mass (m/z): 225.9 [M+H]⁺.

Step 2. Preparation of tert-butyl (2-(2-((4-(tert-butyl)phenyl)amino) ethoxy)ethyl)carbamate (224-2)

A mixture of tert-butyl (2-(2-oxoethoxy)ethyl)carbamate (100 mg, 0.49 mmol), 4-(tert-butyl)aniline (73 mg, 0.49 mmol), Pic-BH₃ (63 mg, 0.59 mmol) in H₂O (9 mL) and CH₃COOH (1 mL) was stirred 5 h at room temperature. Then the reaction was quenched with aqueous NaHCO₃. The organic phases were combined, dried over Na₂SO₄, filtered and concentrated. The target product (122 mg, 74%) was obtained by silica gel chromatography. Mass (m/z): 336.9 [M+H]⁺.

Step 3. Preparation of N-(2-(2-aminoethoxy)ethyl)-4-(tert-butyl)aniline (224)

To a solution of tert-butyl (2-(2-((4-(tert-butyl)phenyl)amino) ethoxy)ethyl)carbamate (122 mg, 0.36 mmol) in HCl in dioxane (3 mL) and in CH₂Cl₂ (3 mL). Then the mixture was stirred for overnight at rt. The reaction was concentrated under vacuum. The residue was purified by prep-TLC to afford the desired product (23 mg, 27%) as a white solid. ¹H NMR (400 MHZ, CD₃OD) δ 7.19 (d, J=8.7 Hz, 2H), 6.68 (d, J=8.7 Hz, 2H), 3.69 (dt, J=12.8, 5.2 Hz, 4H), 3.30-3.27 (m, 2H), 3.14-3.04 (m, 2H), 1.26 (s, 9H). Mass (m/z): 236.9 [M+H]⁺.

### Compound 225

### N1-(4-(4,4-dimethylcyclohexyl)phenyl)propane-1,3-diamine

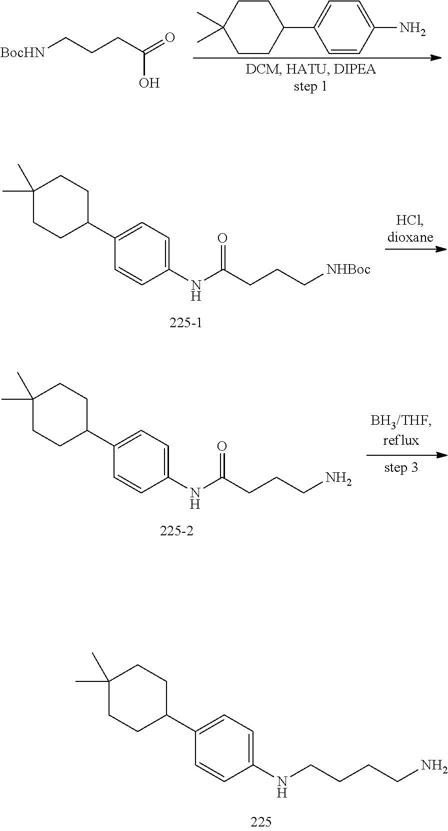

Step 1. Preparation of tert-butyl (3-((4-(4,4-dimethylcyclohexyl)phenyl)amino)-3-oxopropyl)carbamate (225-1)

A mixture of 4-((tert-butoxycarbonyl)amino) butanoic acid (600 mg, 2.95 mmol), 4-(4,4-dimethylcyclohexyl)aniline (500 mg, 2.45 mmol), HATU (1.12 g, 2.95 mmol), and DIPEA (955 mg, 7.4 mmol in DCM (20 mL) was stirred for 2 hours at rt. The reaction was washed with water, dried over Na₂SO₄ and concentrated under vacuum. The residue was purified by prep-TLC to afford the product as light yellow solid (500 mg, 44.7%). Mass (m/z): 288.7 [M-Boc+H]⁺.

Step 2. Preparation of 3-amino-N-(4-(4,4-dimethylcyclohexyl)phenyl)propanamide (225-2)

To a solution of tert-butyl (3-((4-(4,4-dimethylcyclohexyl)phenyl)amino)-3-oxopropyl)carbamate (300 mg, 0.772 mmol) in dioxane/HCl (20 mL, 4M) stirred at rt for 2 hours. The reaction was concentrated under vacuum. The residue was adjusted to pH 8 with aqueous Na₂CO₃ (sat.), exacted with EA (20 mL), dried over Na₂SO₄, filtered and purified by prep-TLC to afford the product as a yellow solid (80 mg, 34.1%). Mass (m/z): 288.7 [M+H]⁺.

Step 3. Preparation of N1-(4-(4,4-dimethylcyclohexyl)phenyl) butane-1,4-diamine (225)

To a solution of 3-amino-N-(4-(4,4-dimethylcyclohexyl)phenyl)propanamide (80 mg, 0.28 mmol) in BH₃-THF (20 mL, 1M) was ad stirred 16 hours at 80° C. The reaction was concentrated under vacuum, washed with water, exacted with EA, dried over Na₂SO₄. The residue was purified by prep-HPLC to afford the desired product as a white solid. (17.8 mg, 23.3%). ¹H NMR (400 MHZ, CDCl₃) δ 7.04 (d, J=8.3 Hz, 2H), 6.56 (d, J=8.3 Hz, 2H), 3.11 (t, J=6.6 Hz, 2H), 2.77 (t, J=6.7 Hz, 2H), 2.35-2.25 (m, 1H), 1.70-1.46 (m, 11H), 1.38-1.19 (m, 3H), 0.95 (d, J=7.3 Hz, 6H). Mass (m/z): 274.7 [M+H]⁺.

### Compound 226

### N-(4-(2-aminopropan-2-yl)cyclohexyl)-4-(4, 4-dimethylcyclohexyl)aniline

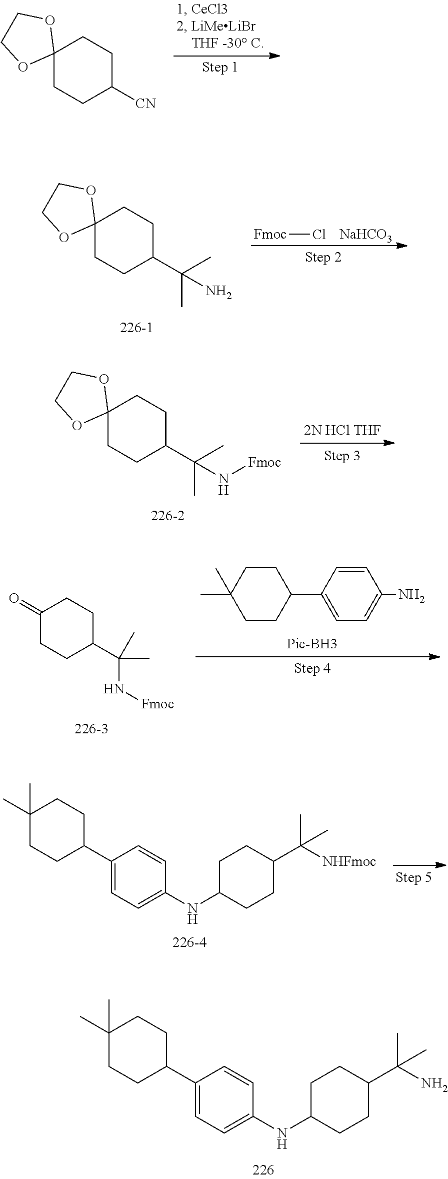

Step 1. Preparation of 2-(1,4-dioxaspiro[4.5]decan-8-yl) propan-2-amine (226-1)

To a solution of 1,4-dioxaspiro[4.5]decane-8-carbonitrile (500 mg, 2.87 mmol), CeCl₃ (1474 mg, 5.74 mmol) and LiMe-LiBr (3 mL, 3 N in THF) in THF (10 mL). The reaction was stirred at −20° C. for 18 hours under N₂ atmosphere. Ethyl acetate was added to the reaction mixture and filtered through Celite, and the filtrate was washed with water and saturated brine. The organic layer was dried over magnesium sulfate and concentrated under reduced pressure. The residue was purified by silica gel column to provide 215 mg (yield: 37.5%) of 226-1 as a yellow oil. Mass (m/z): 200.2 [M+H]⁺.

Step 2. Preparation of (9H-fluoren-9-yl)methyl (2-(1,4-dioxaspiro[4.5]decan-8-yl) propan-2-yl)carbamate (226-2)

To a solution of 226-1 (100 mg, 0.5 mmol) and NaHCO₃ (84 mg, 1 mmol) in H₂O (10 mL) was added Fmoc-Cl (143 mg, 0.55 mmol). The mixture was stirred at 25° C. overnight. Ethyl acetate was added to the reaction mixture and the mixture was filtered through celite. The filtrate was washed with water and saturated brine. The organic layer was dried over magnesium sulfate and concentrated under reduced pressure. The residue was purified by silica gel column to provide 230 mg of 226-3 (yield: 90%) as a yellow solid. Mass (m/z): 444.2 [M+Na]⁺.

Step 3. Preparation of (9H-fluoren-9-yl)methyl (2-(4-oxocyclohexyl) propan-2-yl)carbamate (226-3)

226-2 (700 mg, 1.67 mmol) and HCl in THF (10 mL, 2 N) were placed in a flask stirred at 25° C. for 18 hrs. Excess THF was distilled under vacuum and the residue was purified by silica gel column to provide 400 mg of 226-4 (yield: 63.4%) as a yellow solid. Mass (m/z): 378.0 [M+H]⁺.

Step 4. Preparation of (9H-fluoren-9-yl)methyl (2-(4-((4-(4,4-dimethylcyclohexyl)phenyl)amino)cyclohexyl) propan-2-yl)carbamate (226-4) To a solution of 226-3 (212 mg, 0.6 mmol), 4-(4,4-dimethylcyclohexyl)aniline (113 mg, 0.6 mmol) and Pic-BH₃ (98 mg, 0.9 mmol) in H₂O (9 ml) and HOAc (1 mL). The reaction was stirred at R.T. for 18 hours. The solid was filtered and solvent was removed under vacuum. The residue was purified by silica gel column to provide 250 mg of 226 (yield: 74.0%) as a yellow solid. Mass (m/z): 565.3 [M+H]⁺.

Step 5. Preparation of N-(4-(2-aminopropan-2-yl)cyclohexyl)-4-(4,4-dimethylcyclohexyl)aniline (226)

To a solution of 226-4 (190 mg, 0.34 mmol) and Piperidine (1 mL) in MeOH (5 mL). The reaction was stirred at R.T. for 18 hours. The solid was filtered and solvent was removed under vacuum. The residue was purified by prep-HPLC (column-Xbridge-C18 150×19 mm, 5 μm; Mobile phase: ACN-H₂O (0.1% FA), 40%-60%) to afford 226 (15.4 mg) as a white solid. Mass (m/z): 343.3 [M+H]⁺. ¹H NMR (400 MHZ, CD₃OD) δ 6.98 (d, J=8.4 Hz, 2H), 6.62 (d, J=8.4 Hz, 2H), 3.64 (s, 1H), 2.31-2.23 (m, 1H), 2.04 (d, J=12.8 Hz, 2H), 1.66-1.42 (m, 13H), 1.39-1.33 (m, 2H), 1.31 (s, 6H), 0.96 (d, J=16.0 Hz, 6H).

### Compound 227

### 4-(tert-butyl)-N-(2-methyl-1-(pyrrolidin-1-yl) propan-2-yl)aniline

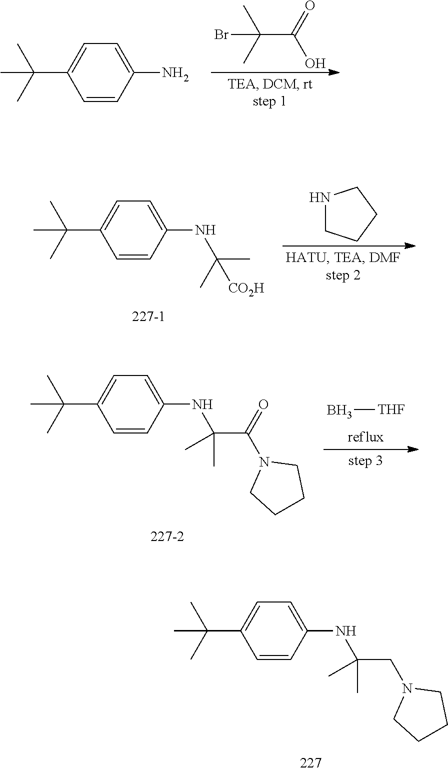

Step 1. Preparation of 2-((4-(tert-butyl)phenyl)amino)-2-methylpropanoic acid (227-1)

A solution of 4-(tert-butyl)aniline (500 mg, 3.35 mmol), 2-bromo-2-methylpropanoic acid (671 mg, 4.02 mmol) and TEA (677 mg, 6.7 mmol) in DCM (30 mL) was stirred overnight at 25° C. LCMS showed the reaction was completed. The reaction was quenched with water (30 mL), extracted with DCM (30 mL*3). The combined organic layers were washed with brine (50 mL), dried over sodium sulfate, filtered and concentrated. The residue was purified by combi-flash with EA/PE (1:1) to afford to give compound 3 (700 mg, 79.9% yield) as a yellow solid. Mass (m/z): 235.9 [M+H]⁺.

2. Preparation of Step 2-((4-(tert-butyl)phenyl)amino)-2-methyl-1-(pyrrolidin-1-yl) propan-1-one (227-2)

A solution of 227-1 (700 mg, 2.97 mmol), pyrrolidine (211 mg, 2.97 mmol), HATU (262 mg, 5.95 mmol) and TEA (600 mg, 5.95 mmol) in DMF (30 mL) was stirred overnight at 25° C. LCMS showed the reaction was completed. The reaction was quenched with water (50 mL), extracted with EA (50 mL*3). The combined organic layers were washed with brine (50 mL), dried over sodium sulfate, filtered and concentrated. The residue was purified by combi-flash with EA/PE (4:1) to afford to give compound 5 (800 mg, 81.6% yield) as yellow oil. Mass (m/z): 289.3 [M+H]⁺.

Step 3. Preparation of 4-(tert-butyl)-N-(2-methyl-1-(pyrrolidin-1-yl) propan-2-yl)aniline (227) A solution of 227-2 (100 mg) in BH₃-THF (20 mL) was stirred 2 h at reflux, quenched with MeOH, concentrated under vacuum and the residue was purified by prep-HPLC (column-Xbridge-C18 150×19 mm, 5 μm; Mobile phase: ACN-H₂O (0.1% FA), 20%-40%) to afford 227-2 (42 mg) as a white solid. ¹H NMR (400 MHZ, CD₃OD) δ 7.30 (br, 2H), 6.83 (br, 2H), 3.94-3.44 (m, 4H), 3.30 (br, 2H), 2.08 (br, 4H), 1.40 (br, 6H), 1.28 (s, 9H). MS (m/z) 275 [M+H]⁺.

### Compound 228

### 3-(+-(4,4-dimethylcyclohexyl)phenyl)amino)cyclobutan-1-ol

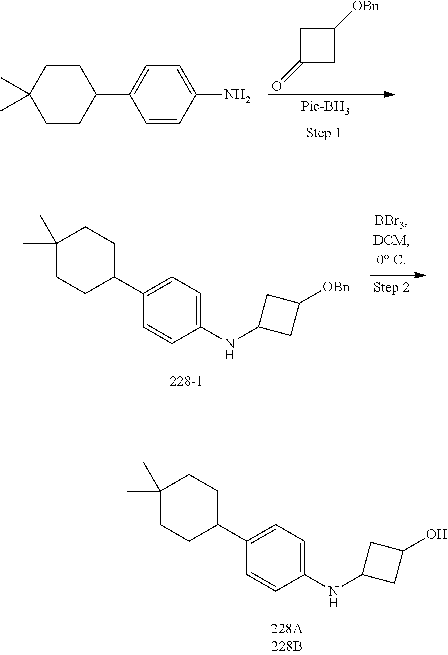 [SMILES: CC1(C)CCC(c2ccc(N)cc2)CC1 ; O=C1CC(OCc2ccccc2)C1 ; CC1(C)CCC(c2ccc(NC3CC(OCc4ccccc4)C3)cc2)CC1 ; CC1(C)CCC(c2ccc(NC3CC(O)C3)cc2)CC1]

Step 1. Preparation of N-(3-(benzyloxy) cyclobutyl)-4-(4,4-dimethylcyclohexyl)aniline (228-1) To a solution of 4-(4,4-dimethylcyclohexyl)aniline (200 mg, 1 mmol), 3-(benzyloxy) cyclobutan-1-one (176 mg, 1 mmol), Pic-BH₃ (87 mg, 0.86 mmol) in H₂O (9 mL) and HOAc (1 mL). The reaction was stirred at R.T. for 18 hours. The reaction mixture was filtered and the filtrate was purified by silica gel column to provide 300 mg of 228-1 (yield: 82.4%) as a yellow solid. Mass (m/z): 364.3 [M+H]⁺.

Step 2. Preparation of 3-((4-(4,4-dimethylcyclohexyl)phenyl)amino)cyclobutan-1-ol (228) To a solution of 228-1 (300 mg, 0.83 mmol) in DCM (10 mL) was added BBr3 (1 mL) at −10° C. and stirred at −10° C. for 18 hours. The reaction mixture was concentrated and the residue was purified by prep-HPLC (column-Xbridge-C18 150×19 mm, 5 μm; Mobile phase: ACN-H₂O (0.1% FA), 25%-40%) to afford 228A (32.8 mg) as a white solid & 228B (31.7 mg) as a white solid. 228A: ¹H NMR (400 MHZ, CD₃OD) δ 6.97 (d, J=8.4 Hz, 2H), 6.50 (d, J=8.4 Hz, 2H), 4.42 (d, J=5.4 Hz, 1H), 3.93 (t, J=4.2 Hz, 1H), 2.32-2.28 (m, 1H), 2.27-2.21 (m, 2H), 2.23-2.14 (m, 2H), 1.69-1.54 (m, 4H), 1.47 (d, J=11.4 Hz, 2H), 1.39-1.26 (m, 2H), 0.96 (d, J=16.4 Hz, 6H). Mass (m/z): 274.3 [M+H]⁺. HPLC: Rt=5.555 min (Column: XBRIDGE 3.5 μm, 2.1*50 mm, Mobile phase: H₂O (0.05% TFA)-ACN (0.05% TFA), ACN from 0 to 60% over 7 minutes, 7-8 min, ACN from 60% to 100%, Flow rate: 0.8 mL/min). 228B: ¹H NMR (400 MHZ, CD₃OD) δ 6.97 (d, J=8.4 Hz, 2H), 6.50 (d, J=8.4 Hz, 2H), 4.43 (t, J=5.4 Hz, 1H), 3.93 (t, J=4.2 Hz, 1H), 2.34-2.13 (m, 5H), 1.66-1.55 (m, 4H), 1.47 (d, J=11.8 Hz, 2H), 1.36-1.23 (m, 2H), 0.96 (d, J=16.4 Hz, 6H). HPLC: Rt=5.570 min (Column: XBRIDGE 3.5 μm, 2.1*50 mm, Mobile phase: H₂O (0.05% TFA)-ACN (0.05% TFA), ACN from 0 to 60% over 7 minutes, 7-8 min, ACN from 60% to 100%, Flow rate: 0.8 mL/min).

### Compound 229

### N-(4-(tert-butyl)phenyl)-2-methylpropane-1,2-diamine

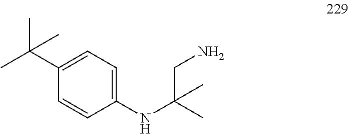 [SMILES: CC(C)(CN)Nc1ccc(C(C)(C)C)cc1]

The title compound 229 (34.7 mg) as a white solid was prepared from 2-((4-(tert-butyl)phenyl)amino)-2-methylpropanamide (150 mg, 0.64 mmol), BH₃-THF (10 mL) and THF (5 mL) according to the procedure for 105. ¹H NMR (400 MHZ, CD₃OD) δ 7.23 (d, 2H), 6.80 (d, 2H), 3.07 (s, 2H), 1.28 (d, 15H). Mass (m/z): 221.0 [M+H]⁺.

### Compound 230

### 2-((4-((4-(tert-butyl)phenyl)amino)cyclohexyl)amino)ethan-1-ol

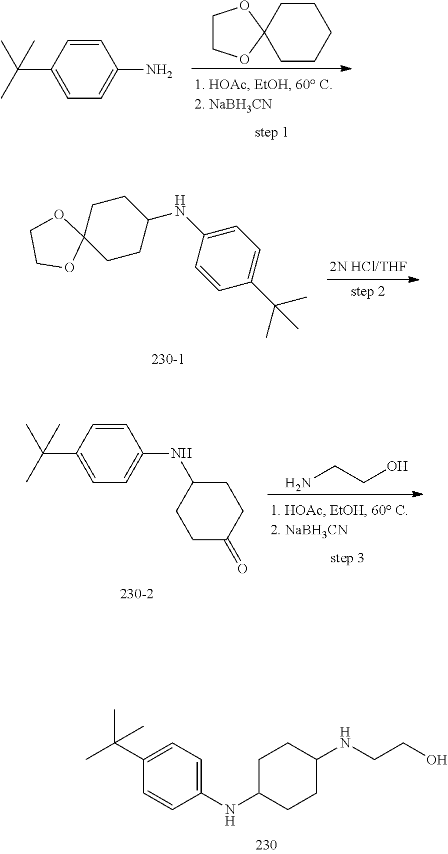

Step 1. Preparation of N-(4-(tert-butyl)phenyl)-1,4-dioxaspiro[4.5]decan-8-amine (230-1) To a solution of 4-(tert-butyl)aniline (1 g, 6.71 mmol) in MeOH (10 mL) was added 1,4-dioxaspiro[4.5]decan-8-one (1.04 g, 6.71 mmol) and the mixture was stirred at 60° C. for 1 h. Then Sodium cyanoborohydride (1.27 g, 20.13 mmol) was added to the mixture. The reaction was stirred for 3 h at R.T. Quenched with water (50 mL), the reaction was extracted by EA (10 mL) for 3 times. The combined organic layers were dried over sodium sulfate, concentrated under vacuum and the residue was purified by flash chromatography to afford the desired product (1.6 g, 82.6%) as a white solid. Mass (m/z): 289.9 [M+H]⁺.

Step 2. Preparation of 4-((4-(tert-butyl)phenyl)amino)cyclohexan-1-one (230-2)

To a solution of N-(4-(tert-butyl)phenyl)-1,4-dioxaspiro[4.5]decan-8-amine (1.6 g, 5.54 mmol) in THF (5 mL) was added HCl in THF (2 N, 5 mL) and the mixture was stirred for 2 h. Quenched with NaHCO₃ (10 mL), the reaction was extracted by EA (10 mL) for 3 times. The combined organic layers were dried over sodium sulfate, concentrated under vacuum to afford the product (0.9 g, 66.2%) as a white solid. Mass (m/z): 246.2 [M+H]⁺.

Step 3. Preparation of 2-((4-((4-(tert-butyl)phenyl)amino)cyclohexyl)amino)ethan-1-ol (230) The desired product 230 (113 mg, 95%) as a yellow solid was prepared from 4-((4-(tert-butyl)phenyl)amino)cyclohexan-1-one (100 mg, 0.41 mmol), MeOH (5 mL), 2-aminoethan-1-ol (25 mg, 0.41 mmol), acetic acid (2.46 mg, 0.041 mmol) and sodium cyanoborohydride (77.49 mg, 12.3 mmol) according to the procedure for 5. ¹H NMR (400 MHz, DMSO-d₆) δ 7.06 (d, J=8.8 Hz, 2H), 6.49 (dd, J=13.6 Hz, 8.4 Hz, 2H), 5.13 (s, 1H), 3.49 (s, 3H), 3.20 (m, 1H), 2.67 (m, 2H), 2.51 (s, 1H), 1.95 (t, J=14.0 Hz, 2H), 1.69-1.49 (m, 3H), 1.20 (s, 12H). Mass (m/z): 290.9 [M+H]⁺.

### Compound 231

### N1-(4-(tert-butyl)phenyl)-4-methylcyclohexane-1,4-diamine

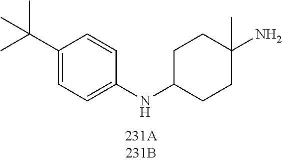 [SMILES: CC1(N)CCC(Nc2ccc(C(C)(C)C)cc2)CC1]

The title compound 231A and 231B were prepared according to the procedure for compound 24. The crude residue was purified by preparative HPLC (Column: X Select-CSH-Prep 5 μm OBD, 19*150 mm; ACN/water (0.5% TFA)=10%-30%-95%-95%-0, 0 min-7 min-7.5 min-8.5 min-10 min) to afford compound 231A (Rt=6.49 min) in 5.7% yield as a white solid and 231B (Rt=5.60 min) in 8.7% yield as a white solid. 231A: ¹H NMR (400 MHZ, DMSO-d₆) δ 7.90 (s, 2H), 7.06 (d, J=7.4 Hz, 2H), 6.61-6.36 (m, 2H), 5.06 (s, 1H), 3.26-3.18 (m, 1H), 1.86-1.71 (m, 4H), 1.57-1.49 (m, 4H), 1.23 (s, 3H), 1.17 (s, 9H). Mass (m/z): 261.3 [M+H]⁺. 231B: ¹H NMR (400 MHZ, DMSO-d₆) δ 7.95-7.72 (m, 3H), 7.28-7.03 (m, 2H), 6.90-6.61 (m, 2H), 3.26-3.11 (m, 1H), 1.87-1.78 (m, 2H), 1.75-1.66 (m, 2H), 1.60-1.50 (m, 2H), 1.45-1.33 (m, 2H), 1.24 (s, 3H), 1.20 (s, 9H). Mass (m/z): 261.3 [M+H]⁺.

### Compound 232

### N1-(4-(tert-pentyl)phenyl)cyclohexane-1,4-diamine

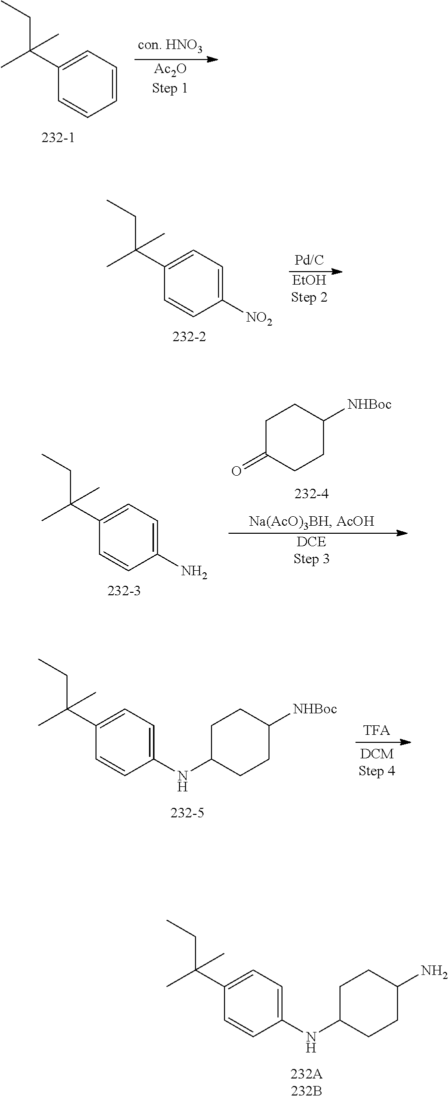

Step 1. Preparation of 1-nitro-4-(tert-pentyl) benzene (232-2)

To a stirred solution of tert-pentylbenzene (388 mg, 2.0 mol) in acetic anhydride (5.0 mL) at −5° C. was added con. HNO₃ (0.3 mL) slowly. The reaction was maintained at 0° C. for 1 hour and then room temperature for 18 hours. Water (10 mL) was added. The reaction was extracted with EtOAc (3×10 mL), dried over Na₂SO₄ and concentrated in vacuum. The residue was purified by prep-TLC (DCM/PE=1/10) to afford the title compound (230 mg, 60%).

Step 2. Preparation of 4-(tert-pentyl)aniline (232-3)

To a solution of 1-nitro-4-(tert-pentyl) benzene (230 mg, 1.2 mmol) in EtOH (10 mL) was added 10% Pd/C (12.6 mg, 12 μmol). Then the reaction was stirred overnight at room temperature (RT) under an atmosphere of hydrogen. Pd/C was filtrated out. The filtrate was concentrated under vacuum. The residue was purified by prep-TLC (EA/PE=1/3) to afford the desired product as a yellow solid (1.8 g, 81.8%). Mass (m/z): 164.3 [M+H]⁺.

Step 3. Preparation of tert-butyl (4-((4-(tert-pentyl)phenyl)amino)cyclohexyl)carbamate (232-5)

The title compound 232-5 (120 mg) was prepared in a total yield of 50.0% as a crude yellow solid from 4-(tert-pentyl)aniline (110 mg, 0.67 mmol), tert-butyl (4-oxocyclohexyl)carbamate (285 mg, 1.34 mmol) and Na(AcO)3BH (283 mg, 1.34 mmol) according to the procedure for 24-1. Mass (m/z): 361.3 [M+H]⁺.

Step 4. N1-(4-(tert-pentyl)phenyl)cyclohexane-1,4-diamine (232)

The title compound 232A, 232B was prepared according to the procedure for compound 28. The crude residue was purified by preparative HPLC (Column: X Select-CSH-Prep 5 μm OBD, 19*150 mm; ACN/water (0.5% TFA)=10%-33%-95%-95%-10%, 0 min-7 min-7.5 min-8.5 min-10 min) to afford compound 232A (Rt=6.23 min) in 14.9% yield as a white solid and 232B (Rt=5.05 min) in 34.7% yield as a white solid. 231A: ¹H NMR (400 MHZ, DMSO-d₆) δ 7.74 (s, 3H), 7.04 (d, J=8.0 Hz, 2H), 6.59 (s, 2H), 3.41-3.32 (m, 1H), 3.16-3.03 (m, 1H), 1.78-1.57 (m, 8H), 1.50 (q, J=7.4 Hz, 2H), 1.13 (s, 6H), 0.57 (t, J=7.4 Hz, 3H). Mass (m/z): 261.3 [M+H]⁺. 231B: ¹H NMR (400 MHZ, DMSO-d₆) δ 7.79 (s, 3H), 7.32-7.04 (m, 2H), 6.92-6.50 (m, 2H), 3.28-3.09 (m, 1H), 3.03-2.90 (m, 1H), 1.93 (t, J=10.8 Hz, 4H), 1.52 (q, J=7.6 Hz, 2H), 1.40-1.12 (m, 10H), 0.57 (t, J=7.4 Hz, 3H). Mass (m/z): 261.3 [M+H]⁺.

### Compound 233

### 4-((4-(tert-butyl)phenyl)amino)-3-methylcyclohexan-1-ol

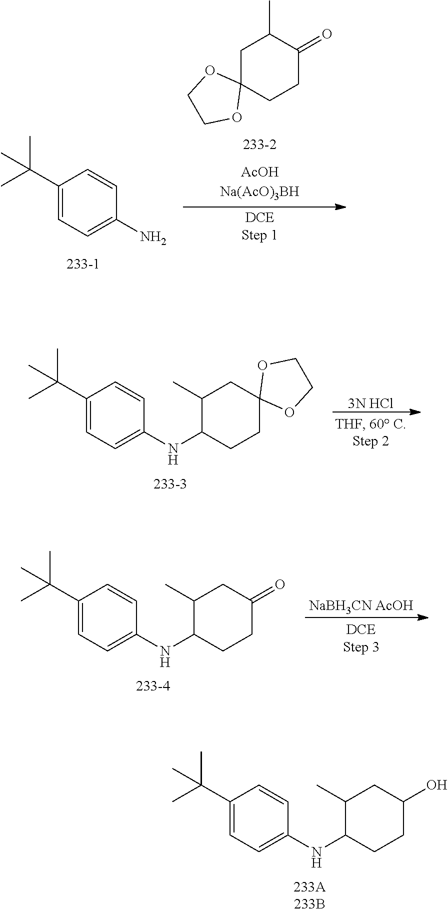

Step 1. Preparation of N-(4-(tert-butyl)phenyl)-7-methyl-1,4-dioxaspiro[4.5]decan-8-amine (233-3)

The title compound 233-3 (350 mg) was prepared in a total yield of 92.3% as a yellow solid from 4-(tert-pentyl)aniline (1.86 mg, 1.25 mmol), 7-methyl-1,4-dioxaspiro[4.5]decan-8-one (319 mg, 1.88 mmol) and Na(AcO)₃BH (399 mg, 1.88 mmol) according to the procedure for 1-1. Mass (m/z): 304.3 [M+H]⁺.

Step 2. Preparation of 4-((4-(tert-butyl)phenyl)amino)-3-methylcyclohexan-1-one (233-4)

A solution of N-(4-(tert-butyl)phenyl)-7-methyl-1,4-dioxaspiro[4.5]decan-8-amine (350 mg, 1.16 mmol) in 10 mL tetrahydrofuran and 10 mL of 1 M hydrochloric acid was heated to 60° C. for 30 min. After cooling to 0° C., the PH of the solution was adjusted to 8-9 with sodium carbonate solution. Then the mixture was extracted by DCM (20 mL×3). The combined organic layers were washed with water (20 mL), dried over Na₂SO₄ and concentrated to afford the desired product as a yellow solid (280 mg, 93.6%). Mass (m/z): 260.3 [M+H]⁺.

Step 3. Preparation of 4-((4-(tert-butyl)phenyl)amino)-3-methylcyclohexan-1-ol (233)

To a solution of 4-((4-(tert-butyl)phenyl)amino)-3-methylcyclohexan-1-one (130 mg, 0.6 mmol) in DCE (10 mL) was added NaBH₃CN (62 g, 1 mmol) and 5 drops of AcOH. The mixture was stirred at rt for 3 h. Then the reaction was washed with water (10 mL), dried over Na₂SO₄, filtered and concentrated in vacuum. The residue was purified by prep-TLC (MeOH/DCM=1/10) to afford compound 233A in 15.9% yield as a light yellow solid and 233B in 11.4% yield as a light yellow solid. 233A: ¹H NMR (400 MHZ, DMSO-d₆) δ 7.23-7.03 (m, 2H), 6.96-6.61 (m, 2H), 5.31 (s, 2H), 3.50-3.36 (m, 2H), 1.81-1.68 (m, 2H), 1.56-1.28 (m, 5H), 1.19 (s, 10H), 0.93 (d, J=6.9 Hz, 3H). Mass (m/z): 262.3 [M+H]⁺. HPLC: Rt=6.485 mins (Agilent, poroshell 120, SB—C_{18 2.7} μm, 4.6×50 mm, ACN/Water (0.1% FA)=5%-5%-95%-95%-95%-5%, 0-0.5 min-10 min-10.5 min-12.0 min). 233B: ¹H NMR (400 MHZ, DMSO-d₆) δ 7.87-6.30 (m, 5H), 3.41-3.32 (m, 1H), 2.90 (qd, J=13.4, 12.2, 6.8 Hz, 1H), 2.03-1.43 (m, 5H), 1.20 (s, 9H), 1.15-0.93 (m, 7H). Mass (m/z): 262.3 [M+H]⁺. HPLC: Rt=5.229 mins (Agilent, poroshell 120, SB—C18 2.7 μm, 4.6×50 mm, ACN/Water (0.1% FA)=5%-5%-95%-95%-95%-5%, 0-0.5 min-10 min-10.5 min-12.0 min).

### Compound 234

### 3-((4-(tert-butyl)phenyl)amino) pentane-1,5-diol

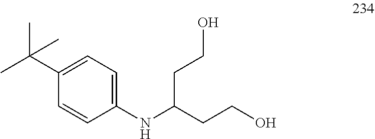 [SMILES: CC(C)(C)c1ccc(NC(CCO)CCO)cc1]

The title compound 234 (72.7 mg) was prepared in a total yield of 58% as a white solid from 4-(tert-butyl)aniline (75 mg, 0.5 mmol) according to the procedure for compound 4. ¹H NMR (400 MHZ, DMSO-d₆) δ 7.05 (d, J=8.4 Hz, 2H), 6.51 (d, J=8.4 Hz, 2H), 5.07 (s, 1H), 4.39 (s, 2H), 3.46 (t, J=68.4 Hz, 4H), 3.35 (m, 1H), 1.59 (m, 4H), 1.20 (s, 9H). Mass (m/z): 252.3 [M+H]⁺.

### Compound 235

### N-(4-(aminomethyl)cyclohexyl)-2,3-dihydro-1H-inden-5-amine

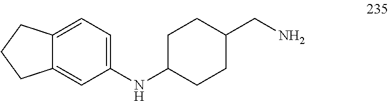 [SMILES: NCC1CCC(Nc2ccc3c(c2)CCC3)CC1]

The title compound 235 (131.4 mg) was prepared in a total yield of 90.3% as a white solid with 1:2 mixture by NMR from tert-butyl (4-((2,3-dihydro-1H-inden-5-yl)amino)cyclohexyl)carbamate (205 mg, 0.596 mmol), TFA (1 mL), and DCM (10 mL) according to the procedure for 24. ¹H NMR (400 MHZ, DMSO-d₆) δ 6.84 (dd, J=8.0, 3.2 Hz, 1H), 6.47-6.25 (m, 2H), 5.06 (d, J=24.0 Hz, 1H), 3.35 (d, J=27.2 Hz, 1H), 2.73-2.53 (m, 6H), 1.98-1.73 (m, 3H), 1.61-1.35 (m, 6H), 1.01 (q, J=9.6 Hz, 1H). Mass (m/z): 245.3 [M+H]⁺.

### Compound 236

### N1-(5,6,7,8-tetrahydronaphthalen-2-yl)cyclohexane-1,4-diamine

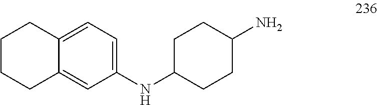 [SMILES: NC1CCC(Nc2ccc3c(c2)CCCC3)CC1]

The title compound 236 (93.7 mg) was prepared in a total yield of 73.4% as a white solid with 1:2 mixture from tert-butyl by H NMR (4-((5,6,7,8-tetrahydronaphthalen-2-yl)amino)cyclohexyl)carbamate (180 mg, 0.523 mmol), TFA (1 mL), and DCM (10 mL) according to the procedure for 24. ¹H NMR (400 MHZ, DMSO-d₆) δ 6.74 (d, J=8.0 Hz, 1H), 6.46-6.20 (m, 2H), 5.03 (s, 1H), 3.00 (d, J=54.4 Hz, 2H), 2.57 (d, J=18.4 Hz, 4H), 1.99 (d, J=11.6 Hz, 2H), 1.75 (d, J=15.2 Hz, 3H), 1.66 (p, J=3.2 Hz, 4H), 1.59 (d, J=12.4 Hz, 1H), 1.46 (q, J=12.4 Hz, 1H), 1.18 (t, J=18.8 Hz, 1H). Mass (m/z): 245.3 [M+H]⁺.

### Compound 237

### 4-(tert-butyl)-N-(3-((methylamino)methyl)cyclobutyl)aniline

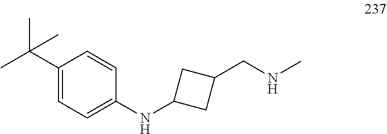 [SMILES: CNCC1CC(Nc2ccc(C(C)(C)C)cc2)C1]

The title compound 237 (17.9 mg) was prepared in a total yield of 25.3% as an orange solid from 3-((4-(tert-butyl)phenyl)amino)-N-methylcyclobutane-1-carboxamide (75 mg, 0.288 mmol), LiAlH₄ (43 mg, 1.152 mmol) and THF (10 mL) according to the procedure for 23. ¹H NMR (400 MHZ, DMSO-d₆) δ 7.73 (s, 1H), 7.09-6.96 (m, 2H), 6.46-6.30 (m, 2H), 5.65 (dd, J=13.6, 7.2 Hz, 1H), 3.74 (dq, J=84.4, 8.0, 7.6 Hz, 1H), 2.69-2.56 (m, 1H), 2.53 (dd, J=10.8, 4.4 Hz, 3H), 2.42-2.32 (m, 2H), 1.98-1.83 (m, 2H), 1.16 (s, 9H). Mass (m/z): 247.3 [M+H]⁺.

### Compound 238

### N1-(naphthalen-1-yl)cyclohexane-1,4-diamine

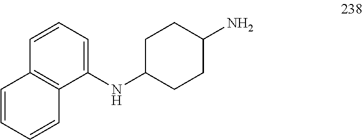 [SMILES: NC1CCC(Nc2cccc3ccccc23)CC1]

The title compound 238 (119.1 mg) was prepared in a total yield of 85% as a pink solid with 1:2 mixture by ¹H NMR from tert-butyl (4-(naphthalen-1-ylamino)cyclohexyl)carbamate (198 mg, 0.582 mmol), TFA (1 mL), and DCM (10 mL) according to the procedure for 24. ¹H NMR (400 MHZ, DMSO-d₆) δ 8.38 (s, 1H), 8.32-8.24 (m, 1H), 8.19 (dd, J=20.8, 8.0 Hz, 1H), 7.70 (td, J=8.4, 1.6 Hz, 1H), 7.42-7.30 (m, 2H), 7.23 (td, J=8.0, 5.6 Hz, 1H), 7.06 (dd, J=17.2, 8.0 Hz, 1H), 6.52 (t, J=7.2 Hz, 1H), 5.89-5.39 (m, 1H), 3.17-2.88 (m, 1H), 2.13-2.01 (m, 2H), 1.95 (dq, J=8.8, 4.4 Hz, 1H), 1.84 (t, J=10.0 Hz, 1H), 1.78-1.72 (m, 1H), 1.70-1.61 (m, 1H), 1.59-1.46 (m, 1H), 1.44-1.31 (m, 1H). Mass (m/z): 241.3 [M+H]⁺.

### Compound 239

### N1-(4-phenoxyphenyl)cyclohexane-1,4-diamine

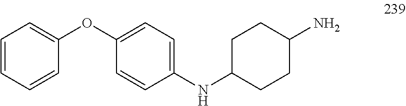 [SMILES: NC1CCC(Nc2ccc(Oc3ccccc3)cc2)CC1]

The title compound 239 (93.2 mg) was prepared in a total yield of 70.6% as a white solid with 1:2 mixture by ¹H NMR from tert-butyl (4-((4-phenoxyphenyl)amino)cyclohexyl)carbamate (179 mg, 0.469 mmol), TFA (1 mL), and DCM (10 mL) according to the procedure for 24. ¹H NMR (400 MHZ, DMSO-d₆) δ 7.29 (tt, J=7.2, 2.0 Hz, 2H), 7.04-6.94 (m, 1H), 6.89-6.77 (m, 4H), 6.71-6.55 (m, 2H), 5.32 (d, J=5.0 Hz, 1H), 3.09 (s, 1H), 2.95 (s, 1H), 2.01 (d, J=11.2 Hz, 3H), 1.78 (qd, J=12.0, 10.8, 7.2 Hz, 3H), 1.61 (dt, J=13.6, 7.2 Hz, 1H), 1.54-1.40 (m, 1H). Mass (m/z): 283.3 [M+H]⁺.

### Compound 240

### N1-(3,4-dimethylphenyl)cyclohexane-1,4-diamine

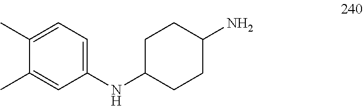 [SMILES: Cc1ccc(NC2CCC(N)CC2)cc1C]

The title compound 240 (80.2 mg) was prepared in a total yield of 65.6% as a white solid with 1:2 mixture by ¹H NMR from tert-butyl (4-((3,4-dimethylphenyl)amino)cyclohexyl)carbamate (178 mg, 0.560 mmol), TFA (1 mL), and DCM (10 mL) according to the procedure for 24. ¹H NMR (400 MHZ, DMSO-d₆) δ 6.76 (dd, J=8.0, 5.2 Hz, 1H), 6.36 (dd, J=18.0, 2.4 Hz, 1H), 6.27 (ddd, J=15.6, 8.0, 2.4 Hz, 1H), 4.98 (d, J=7.6 Hz, 1H), 3.37-2.75 (m, 2H), 2.05 (d, J=2.0 Hz, 3H), 2.00 (d, J=2.4 Hz, 3H), 1.97-1.86 (m, 2H), 1.73-1.47 (m, 4H), 1.36 (qd, J=13.6, 12.4, 3.6 Hz, 1H), 1.15-1.02 (m, 1H). Mass (m/z): 219.3 [M+H]⁺.

### Compound 241

### N-(3-(aminomethyl)cyclopentyl)-4-(tert-butyl)aniline

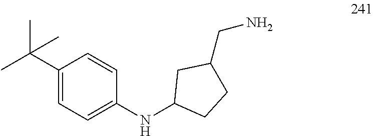 [SMILES: CC(C)(C)c1ccc(NC2CCC(CN)C2)cc1]

The title compound 241 (4.2 mg) was prepared in a total yield of 5.4% as a pink solid from 3-((4-(tert-butyl)phenyl)amino)cyclopentane-1-carboxamide (83 mg, 0.319 mmol), LiAlH₄ (47 mg, 1.276 mmol) and THF (10 mL) according to the procedure for 23. ¹H NMR (400 MHZ, DMSO-d₆) δ 7.06 (d, J=8.0 Hz, 2H), 6.50 (s, 2H), 3.67 (dt, J=12.8, 6.3 Hz, 1H), 2.71 (d, J=16.8 Hz, 2H), 2.29-1.91 (m, 3H), 1.84 (ddd, J=14.0 9.2, 5.6 Hz, 1H), 1.77-1.66 (m, 1H), 1.46-1.35 (m, 2H), 1.17 (s, 9H). Mass (m/z): 247.3 [M+H]⁺.

### Compound 242

### N1-(4-(tert-butyl)-3-fluorophenyl)cyclohexane-1,4-diamine 2, 2,2-trifluoroacetate

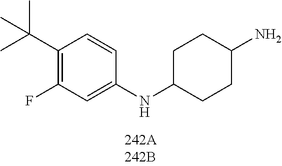 [SMILES: CC(C)(C)c1ccc(NC2CCC(N)CC2)cc1F]

The title compound 242A and 242B were prepared according to the procedure for compound 20. The residue was purified by preparative HPLC (Column: X Select-CSH-Prep 5 μm OBD, 19*150 mm; ACN/water (0.5% TFA)=25%-30%-95%-95%-10%, 0 min-10 min-10.5 min-11.5 min-13 min) to afford compound 242A (Rt=8.13 min) in 19.0% yield as a white solid and 242B (Rt=8.63 min) in 18.1% yield as a white solid. 242A: ¹H NMR (400 MHZ, DMSO-d₆) δ 7.80 (s, 3H), 6.97 (dd, J=10.2, 8.2 Hz, 1H), 6.37-6.21 (m, 2H), 3.09 (d, J=22.1 Hz, 1H), 2.99 (d, J=5.1 Hz, 1H), 2.06-1.87 (m, 4H), 1.51-1.33 (m, 2H), 1.24 (d, J=0.8 Hz, 9H), 1.15 (q, J=11.4 Hz, 2H). Mass (m/z): 265.3 [M+H]⁺. 242B: ¹H NMR (400 MHZ, DMSO-d₆) δ 7.80 (s, 3H), 7.09 (dt, J=8.7, 1.1 Hz, 1H), 6.56 (q, J=2.5 Hz, 1H), 6.44 (dt, J=8.7, 2.2 Hz, 1H), 3.07 (dd, J=14.5, 7.3 Hz, 1H), 2.97 (d, J=4.8 Hz, 1H), 2.00-1.84 (m, 4H), 1.44-1.34 (m, 2H), 1.32 (s, 9H), 1.13 (q, J=11.6 Hz, 2H). Mass (m/z): 265.3 [M+H]⁺.

### Compound 243

### 1-(2-(4-((4-(4,4-dimethylcyclohexyl)phenyl)amino)cyclohexylethyl) urea

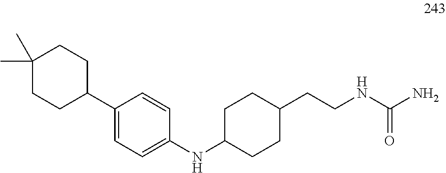 [SMILES: CC1(C)CCC(c2ccc(NC3CCC(CCNC(N)=O)CC3)cc2)CC1]

The desired product 243 (10.1 mg) as a white powder with 1:1 mixture by ¹H NMR in a yield of 24.8% prepared from was of N-(4-(2-aminoethyl)cyclohexyl)-4-(4,4-dimethylcyclohexyl)aniline (36 mg, 0.11 mmol), DMSO (5 mL), phenyl carbamate (18 mg, 0.13 mmol) according to the procedure for 116. ¹H NMR (400 MHZ, DMSO-d₆) δ 6.91 (d, J=8.0 Hz, 2H), 6.49 (dd, J=19.6, 7.9 Hz, 2H), 5.86 (s, 1H), 5.34 (s, 2H), 5.13 (d, J=27.3 Hz, 1H), 2.97 (d, J=6.7 Hz, 2H), 2.20 (d, J=6.4 Hz, 1H), 1.97 (t, J=14.1 Hz, 2H), 1.73 (d, J=12.5 Hz, 1H), 1.60-1.49 (m, 6H), 1.49-1.36 (m, 6H), 1.30 (dd, J=14.1, 8.1 Hz, 5H), 1.03 (dt, J=21.9, 11.7 Hz, 2H), 0.94 (s, 3H), 0.92 (s, 3H). Mass (m/z): 372.6 [M+H]⁺.

### Compound 244

### (R)—N-(2-(4-((4-(4,4-dimethylcyclohexyl)phenyl)amino)cyclohexyl)ethyl)-2,6-dioxohexahydro pyrimidine-4-carboxamide

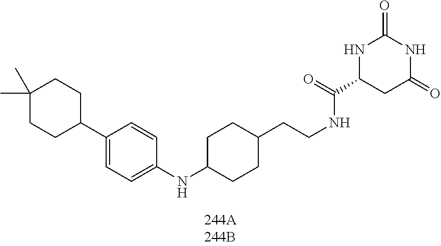 [SMILES: CC1(C)CCC(c2ccc(NC3CCC(CCNC(=O)[C@H]4CC(=O)NC(=O)N4)CC3)cc2)CC1]

The title compound 244 (244A and 244B) was prepared in a yield of 18.6% as a white powder from N-(4-(2-aminoethyl)cyclohexyl)-4-(4,4-dimethylcyclohexyl)aniline (50 mg, 0.15 mmol), (R)-2,6-dioxohexahydropyrimidine-4-carboxylic acid (29 mg, 0.18 mmol) according to the procedure for 1. The mixture was purified by preparative HPLC (Column: X Select-CSH-Prep 5 μm OBD, 19*150 mm; ACN/water (0.5% TFA)=5%-5%-95%-95%-5%, 0 min-1 min-10 min-11 min-15.0 min) afford compound 244A (Rt=6.11 min) as a white solid and compound 244B (Rt=5.90 min) as a white solid. ¹H NMR (400 MHZ, DMSO-d₆) δ 10.02 (d, J=1.9 Hz, 1H), 8.02 (t, J=5.6 Hz, 1H), 7.64-7.51 (m, 1H), 6.93 (d, J=8.0 Hz, 2H), 6.52 (d, J=8.1 Hz, 2H), 5.18 (s, 1H), 3.93 (dt, J=7.1, 3.5 Hz, 1H), 3.39 (s, 1H), 3.15-3.03 (m, 2H), 2.83 (dd, J=16.6, 7.2 Hz, 1H), 2.43 (dd, J=16.2, 3.2 Hz, 1H), 2.21 (dq, J=10.5, 5.8, 5.4 Hz, 1H), 1.53 (dt, J=8.0, 3.3 Hz, 7H), 1.49-1.42 (m, 4H), 1.42-1.34 (m, 5H), 1.29 (dd, J=12.3, 5.2 Hz, 3H), 0.95 (s, 3H), 0.93 (s, 3H). Mass (m/z): 469.3 [M+H]⁺.

### Compound 245

### 1-(2-(4-((4-(tert-butyl)phenyl)amino)piperidin-1-yl)-2-oxoethyl)-4-methylpiper-azin-2-one

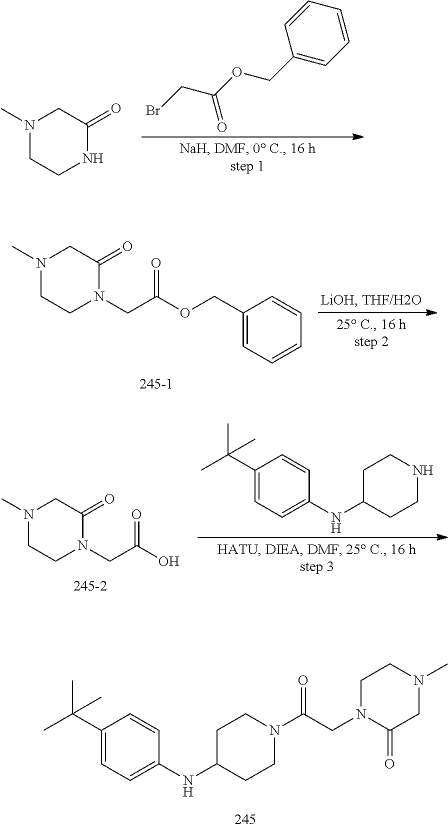

Step 1. benzyl 2-(4-methyl-2-oxopiperazin-1-yl) acetate (245-1)

A mixture of 4-methylpiperazin-2-one (500 mg, 4.38 mmol), NaH (210.26 mg, 5.25 mmol) in THF (5 mL) was stirred 1 hours at 0° C. Then it was added benzyl 2-bromoacetate (1.1 g, 4.81 mmol). It was quenched by H₂O (30 mL) and extracted with DCM (3×30 mL). The organic layers were combined, washed with brine NaCl (2×30 mL) and dried with MgSO₄, filtered and concentrated to give product (800 mg, yield: 55.7%) as yellow oil. Mass (m/z): 262.9 [M+H]⁺.

Step 2. 2-(4-methyl-2-oxopiperazin-1-yl) acetic acid (245-2)

To a solution of 245-1 (300 mg, 1.14 mmol), LiOH (273.89 mg, 11.43 mmol) in THF/H₂O=1:1 (5 mL). Then the mixture was stirred overnight at 25° C. It was quenched by H₂O (300 mL) and extracted with EA (3×300 mL). The organic layers were combined, washed with brine NaCl (2×300 mL) and dried with MgSO₄, filtered and concentrated to afford the target product (100 mg, yield: 40.6%) as yellow oil.

Step 3. Preparation of 1-(2-(4-((4-(tert-butyl)phenyl)amino)piperidin-1-yl)-2-oxoethyl)-4-methylpiperazin-2-one (245)

To a solution of 245-2 (80 mg, 0.46 mmol), N-(4-(tert-butyl)phenyl)piperidin-4-amine (107.96 mg, 0.46 mmol) in DMF (5 ml) was added HATU (88.33 mg, 0.23 mmol), and DIEA (600.45 mg, 4.64 mmol). Then the mixture was stirred overnight at rt. The reaction was concentrated under vacuum. The residue was purified by prep-TLC to afford the desired product 245 (19.8 mg) as a white solid. ¹H NMR (400 MHZ, CD₃OD) δ 7.16 (d, J=9.2 Hz, 2H), 6.63 (dd, J=9.2 Hz, 2H), 4.34 (d, J=16.0 Hz, 2H), 4.20 (d, J=16.4 Hz, 1H), 3.85 (d, J=14.0 Hz, 1H), 3.59-3.47 (m, 1H), 3.41 (t, J=5.4 Hz, 2H), 3.18 (s, 2H), 2.90 (t, J=12.4 Hz, 1H), 2.79 (s, 2H), 2.38 (d, J=1.6 Hz, 3H), 2.03 (dd, J=19.4, 14.8 Hz, 2H), 1.45-1.26 (m, 2H), 1.23 (s, 9H). Mass (m/z): 315.2 [M+H]⁺.

### Compound 246

### 4-(tert-butyl)-N-(2-(4,4-dimethylpiperidin-1-yl)ethyl)aniline

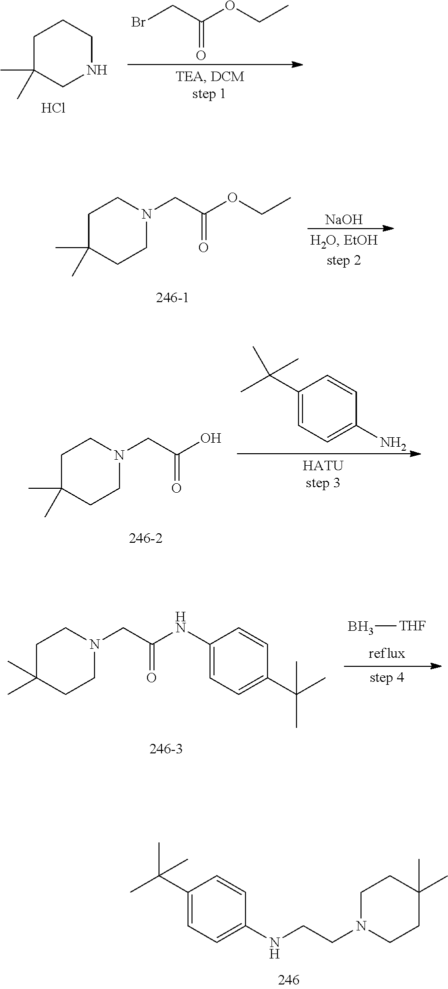

Step 1. ethyl 2-(4,4-dimethylpiperidin-1-yl) acetate (246-1)

A mixture of 4,4-dimethylpiperidine (500 mg, 4.41 mmol) and TEA (1.78 g, 17.66 mmol) in DCM (10 mL) was stirred overnight at 25° C. Then it was added ethyl 2-bromoacetate (811.39 mg, 4.85 mmol). It was quenched by H₂O (300 mL) and extracted with DCM (3×300 mL). The organic layers were combined, washed with brine NaCl (2×300 mL) and dried with MgSO₄, filtered and concentrated to give the target product (600 mg, 61.3%) as a yellow oil.

Step 2. 2-(4,4-dimethylpiperidin-1-yl) acetic acid (246-2)

To a solution of 246-1 (600 mg, 3.01 mmol), NaOH (361.26 mg, 9.03 mmol) in H₂O/EtOH=2:1 (15 ml). Then the mixture was stirred overnight at 50° C. It was quenched by H₂O (300 mL) and extracted with EA (3×300 mL). The organic layers were combined, washed with brine NaCl (2×300 mL) and dried with MgSO₄, filtered and concentrated to give the target product (300 mg, 52.3%) as yellow oil.

Step 3. N-(4-(tert-butyl)phenyl)-2-(4,4-dimethylpiperidin-1-yl) acetamide (246-3)

To a solution of 246-2 (150 mg, 0.87 mmol), and 4-(tert-butyl)aniline (130.73 mg, 0.87 mmol) in DMF (5 mL) was added HATU (499.62 mg, 1.31 mmol), TEA (433.21 mg, 4.38 mmol). Then the mixture was stirred overnight at rt. It was quenched by H₂O (100 mL) and extracted with EA (3×100 mL). The organic layers were combined, washed with brine NaCl (2×100 mL) and dried with MgSO₄, filtered and concentrated. The crude product was applied onto a silica gel column (12 g) eluted with PE: EA (5:1) to give the product (300 mg, 90.5%) as a yellow solid. Mass (m/z): 302.9 [M+H]⁺.

Step 4. Preparation of 4-(tert-butyl)-N-(2-(4,4-dimethylpiperidin-1-yl)ethyl)aniline (246) To a solution of 246-3 (160 mg, 0.53 mmol) in THF (5 mL) was added BH₃-THF (5 mL). Then the mixture was stirred overnight at 25° C. The reaction was concentrated under vacuum. The residue was purified by prep-TLC to afford the desired product 246 (39.5 mg) as a white solid. ¹H NMR (400 MHZ, CD₃OD) δ 8.40 (s, 1H), 7.17 (t, J=5.8 Hz, 2H), 6.62 (d, J=8.8 Hz, 2H), 3.49 (t, J=5.6 Hz, 2H), 3.29 (dd, J=4.8, 3.2 Hz, 4H), 3.28 (s, 2H), 1.64 (d, J=5.0 Hz, 4H), 1.27-1.18 (m, 9H), 1.03 (d, J=7.0 Hz, 6H). Mass (m/z): 289.0 [M+H]⁺.

### Compound 247

### 1-(3-(dimethylamino) propyl)-4-((4-(4,4-dimethylcyclohexyl)phenyl)amino)piperidin-2-one

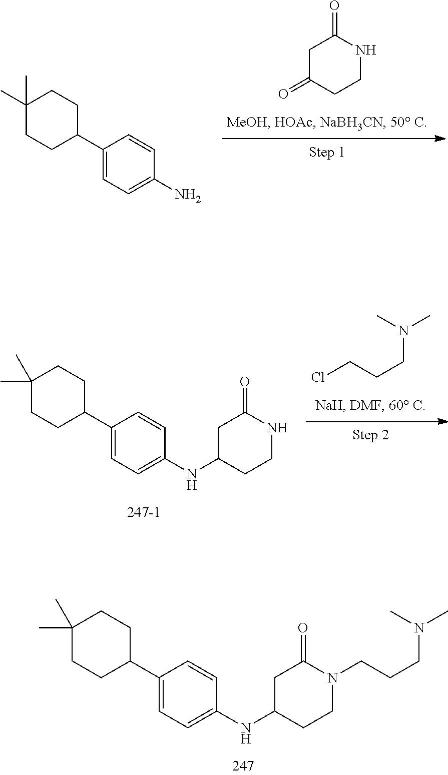

Step 1. Preparation of 4-((4-(4,4-dimethylcyclohexyl)phenyl)amino)piperidin-2-one (247-1) A mixture of 4-(4,4-dimethylcyclohexyl)aniline (200 mg, 0.98 mmol), 122-piperidine-2,4-dione (133.5 mg, 1.18 mmol) and NaBH₃CN (123.6 mg, 1.97 mmol) in MeOH (10 mL) and AcOH (1 drop) was stirred overnight at 50° C. After cooling, the reaction solution was washed with water, and excess MeOH was distilled under vacuum and the residual oil was distilled under vacuum. Then the reaction solution was extracted three times with ethyl acetate (30 mL) and water (30 mL). The organic layers were combined, the solvent was removed under vacuum and the crude was purified through silica gel chromatography (PE: EA=5:1) to give the desired product as oil (220 mg, 44.7%). Mass (m/z): 300.9 [M+H]⁺.

Step 2. Preparation of 1-(3-(dimethylamino) propyl)-4-((4-(4,4-dimethylcyclohexyl)phenyl)amino)piperidin-2-one (247)

The mixture of 4-((4-(4,4-dimethylcyclohexyl)phenyl)amino)piperidin-2-one (132 mg, 0.44 mmol), 3-chloro-N,N-dimethylpropan-1-amine (80.2 mg, 0.66 mmol) and NaH (21.1 mg, 0.88 mmol) in DMF (3 mL) was stirred for 16 hours at 60° C. After reaction was completed, solution was quenched with water and removed under vacuum and the residue was purified by prep-HPLC (column-Xbridge-C18 150×21.2 mm, 5 μm; Mobile phase: ACN-H₂O (0.1% FA), 40%-60%) to afford the desired product 247 (22.2 mg, 12.7%) as brown oil. ¹H NMR (400 MHz, DMSO-d₆) δ 6.95 (d, J=8.4 Hz, 2H), 6.52 (d, J=8.4 Hz, 2H), 5.39 (br, 1H), 3.65 (br, 1H), 3.34-3.24 (m, 4H), 2.58-2.54 (m, 1H), 2.36-2.24 (m, 3H), 2.20 (s, 6H), 2.15-2.03 (m, 2H), 1.67-1.23 (m, 11H), 0.93 (d, J=8.6 Hz, 6H). Mass (m/z): 386.3 [M+H]⁺.

### Compound 248

### 1-(4-((4-(4,4-dimethylcyclohexyl)phenyl)amino)cyclohexyl)urea

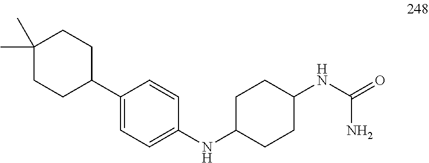 [SMILES: CC1(C)CCC(c2ccc(NC3CCC(NC(N)=O)CC3)cc2)CC1]

The desired product 248 (15.7 mg, 12.0%) as a white solid was prepared from N1-(4-(4,4-dimethylcyclohexyl)phenyl)cyclohexane-1,4-diamine (105 mg, 0.34 mmol), TMSNCO (40.25 mg, 0.35 mmol), TEA (70.7 mg, 0.70 mmol), DMAP (8.5 mg, 0.07 mmol) and DCM (10 mL) according to the procedure for 178. ¹H NMR (400 MHZ, DMSO-d₆) δ 6.92 (d, J=8.3 Hz, 2H), 6.50 (d, J=8.3 Hz, 2H), 5.96 (br, 1H), 5.37 (br, 2H), 5.16 (br, 1H), 3.25 (br, 1H), 2.20 (br, 1H), 1.64-1.23 (m, 17H), 0.93 (d, J=8.4 Hz, 6H). Mass (m/z): 343.9 [M+H]⁺.

### Compound 249

### N-(4-{[4-(4,4-dimethylcyclohexyl)phenyl]amino}cyclohexyl)aminosulfonamide

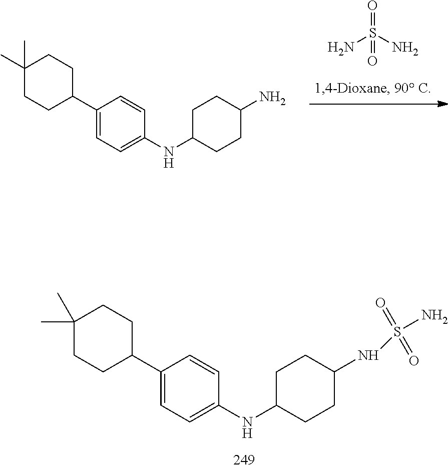 [SMILES: CC1(C)CCC(c2ccc(NC3CCC(N)CC3)cc2)CC1 ; NS(N)(=O)=O ; CC1(C)CCC(c2ccc(NC3CCC(NS(N)(=O)=O)CC3)cc2)CC1]

To a solution of 1-N-[4-(4,4-dimethylcyclohexyl)phenyl]cyclohexane-1,4-diamine (95 mg, 0.32 mmol), and sulfamoylamine (36.5 mg, 0.38 mmol) in 1,4-dioxane (10 mL) was stirred overnight at 90° C. under N₂. After cooling to rt., the reaction was concentrated under vacuum and the residue was purified by prep-HPLC (column-Xbridge-C18 150×21.2 mm, 5 μm; Mobile phase: ACN-H₂O (0.1% FA), 30%-50%) to afford the desired product 249 (13.2 mg, 10.5%) as a white solid. ¹H NMR (400 MHZ, DMSO-d₆) δ 6.92 (d, J=8.3 Hz, 2H), 6.49 (d, J=8.4 Hz, 2H), 6.45 (s, 2H), 6.39 (d, J=5.7 Hz, 1H), 5.09 (br, 1H), 3.25 (br, 2H), 2.28-2.16 (m, 1H), 1.67-1.24 (m, 16H), 0.93 (d, J=8.6 Hz, 6H). Mass (m/z): 380.3 [M+H]⁺.

### Compound 250

### N1-(4-(tert-butyl)phenyl)-3-methylcyclohexane-1,4-diamine

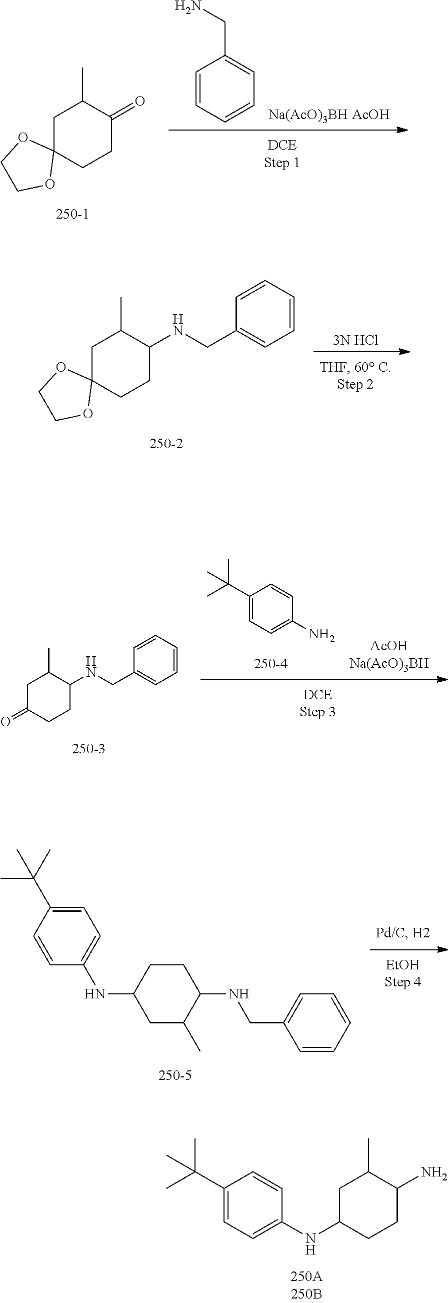

Step 1. Preparation of N-benzyl-7-methyl-1,4-dioxaspiro[4.5]decan-8-amine (250-2)

The title compound 250-2 (420 mg) was prepared in a total yield of 53.6% as a light-yellow oil from 7-methyl-1,4-dioxaspiro[4.5]decan-8-one (510 mg, 3.0 mmol) phenylmethanamine (321 mg, 3.0 mmol) and Na(AcO)₃BH (954 mg, 4.5 mmol) according to the procedure for 24-1. Mass (m/z): 262.2 [M+H]⁺.

Step 2. Preparation of 4-(benzylamino)-3-methylcyclohexan-1-one (250-3)

The title compound 250-3 (185 mg) was prepared in a total yield of 74.0% as a light-yellow oil from 7-methyl-1,4-dioxaspiro[4.5]decan-8-one (300 mg, 1.15 mmol) and 3N HCl (1.0 mL) according to the procedure for 233-4. Mass (m/z): 217.3 [M+H]⁺.

Step 3. Preparation of N1-benzyl-N4-(4-(tert-butyl)phenyl)-2-methylcyclohexane-1,4-diamine (250-5)

The title compound 250-5 (120 mg) was prepared in a total yield of 50.0% as a light-yellow oil from 4-(benzylamino)-3-methylcyclohexan-1-one (150 mg, 0.69 mmol), 4-(tert-butyl)aniline (103 mg, 0.69 mmol) and Na(AcO)3BH (293 mg, 1.38 mmol) according to the procedure for 24-1. Mass (m/z): 351.3 [M+H]⁺.

Step 4. Preparation of N1-(4-(tert-butyl)phenyl)-3-methylcyclohexane-1,4-diamine (250)

The title compound 250A and 250B were prepared according to the procedure for compound 232-3. The crude residue was purified by preparative HPLC (Column: X Select-CSH-Prep 5 μm OBD, 19*150 mm; ACN/water (0.5% TFA)=10%-27%-95%-95%-10%, 0 min-12 min-12.5 min-13.5 min-15 min) to afford compound 250A (Rt=10.17 min) in 4.0% yield as a white solid and 250B (Rt=5.05 min) in 15.3% yield as a white solid. 250A: ¹H NMR (400 MHz, Methanol-d₄) δ 7.15-7.10 (m, 2H), 6.61-6.57 (m, 2H), 3.62-3.58 (m, H), 2.47 (td, J=10.0, 4.0 Hz, 1H), 1.92-1.81 (m, 2H), 1.73-1.55 (m, 3H), 1.37-1.29 (m, 2H), 1.23 (s, 9H), 0.98 (d, J=6.4 Hz, 3H). Mass (m/z): 261.3 [M+H]⁺. 250B: ¹H NMR (400 MHZ, Methanol-d₄) δ 7.16-7.10 (m, 2H), 6.61-6.57 (m, 2H), 3.25-3.18 (m, 1H), 2.95-2.89 (m, 1H), 1.86-1.62 (m, 5H), 1.32-1.20 (m, 11H), 0.94 (d, J=6.8 Hz, 3H). Mass (m/z): 261.3 [M+H]⁺.

### Compound 251

### N1-(4-(4.4-difluorocyclohexyl)phenyl)cyclohexane-1,4-diamine

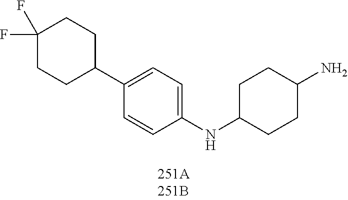 [SMILES: NC1CCC(Nc2ccc(C3CCC(F)(F)CC3)cc2)CC1]

The title compound 251A and 251B were prepared according to the procedure for compound 24. The crude residue was purified by preparative HPLC (Column: X Select-CSH-Prep 5 μm OBD, 19*150 mm; ACN/water (0.5% TFA)=10%-30%-95%-95%-10%, 0 min-10 min-10.5 min-11.5 min-13 min) to afford compound 251A (Rt=8.29 min) in 17.7% yield as a white solid and 251B (Rt=6.36 min) in 8.8% yield as a white solid. 251A: ¹H NMR (400 MHZ, DMSO-d₆) δ 6.87 (d, J=8.6 Hz, 2H), 6.47 (d, J=8.6 Hz, 2H), 5.16 (d, J=7.6 Hz, 1H), 3.26-3.16 (m, 1H), 2.80-2.71 (m, 1H), 2.04-1.72 (m, 7H), 1.62-1.42 (m, 10H). Mass (m/z): 309.3 [M+H]⁺. 251B: ¹H NMR (400 MHZ, DMSO-d₆) δ 6.87 (d, J=8.5 Hz, 2H), 6.43 (d, J=8.6 Hz, 2H), 5.11 (d, J=8.2 Hz, 1H), 3.07-2.94 (m, 1H), 2.48-2.49 (m, 1H), 2.07-1.67 (m, 11H), 1.57-1.48 (m, 3H), 1.11-0.99 (m, 4H). Mass (m/z): 309.3 [M+H]⁺.

### Compound 252

### N1-(4-(tert-butyl)phenyl)-2-methylcyclohexane-1,4-diamine

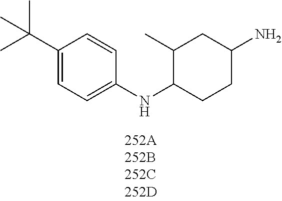 [SMILES: CC1CC(N)CCC1Nc1ccc(C(C)(C)C)cc1]

The title compound 252A, 252B, 252C, 252D were prepared according to the procedure for compound 250. The crude residue was purified by preparative HPLC (Column: X Select-CSH-Prep 5 μm OBD, 19*150 mm; ACN/water (0.5% TFA)=10%-43%-95%-95%-10%, 0 min-10 min-10.5 min-11.5 min-13 min) to afford compound 252A (Rt=9.55 min) in 1.1% yield as a white solid, 252B (Rt=7.47 min) in 2.8% yield as a white solid, 252C (Rt=6.45 min) in 2.7% yield as a white solid and 252D (Rt=5.88 min) in 7.3% yield as a white solid. 252A: ¹H NMR (400 MHZ, Methanol-d₄) δ 7.12-7.07 (m, 2H), 6.61-6.54 (m, 2H), 3.51-3.45 (m, 1H), 2.77-2.63 (m, 1H), 1.98-1.91 (m, 1H), 1.86-1.74 (m, 1H), 1.67-1.56 (m, 2H), 1.47-1.36 (m, 1H), 1.33-1.26 (m, 2H), 1.23 (s, 9H), 0.97 (d, J=7.0 Hz, 3H). Mass (m/z): 261.3 [M+H]⁺. 252B: ¹H NMR (400 MHZ, Methanol-d₄) δ 7.16-7.05 (m, 2H), 6.57-6.46 (m, 2H), 3.14-2.92 (m, 2H), 1.89-1.77 (m, 2H), 1.75-1.54 (m, 3H), 1.52-1.39 (m, 3H), 1.23 (s, 9H), 1.01 (d, J=6.8 Hz, 3H). Mass (m/z): 261.3 [M+H]⁺. 252C: ¹H NMR (400 MHZ, Methanol-d₄) δ 7.10-7.06 (m, 2H), 6.53-6.48 (m, 2H), 2.81-2.67 (m, 2H), 2.11-1.81 (m, 4H), 1.51-1.41 (m, 1H), 1.22 (s, 9H), 1.14-1.02 (m, 2H), 1.00 (d, J=6.4 Hz, 3H). Mass (m/z): 261.3 [M+H]⁺. 252D: ¹H NMR (400 MHZ, Methanol-d₄) δ 7.14-7.09 (m, 2H), 6.62-6.55 (m, 2H), 3.44-3.37 (m, 1H), 2.94-2.84 (m, 1H), 2.32-2.24 (m, 1H), 1.91-1.84 (m, 1H), 1.79-1.66 (m, 2H), 1.59-1.51 (m, 1H), 1.45-1.35 (m, 1H), 1.30-1.19 (m, 10H), 0.88 (d, J=7.2 Hz, 3H). Mass (m/z): 261.3 [M+H]⁺.

### Compound 253

### N1-(4-(tert-butyl)-3-hexylphenyl)cyclohexane-1,4-diamine

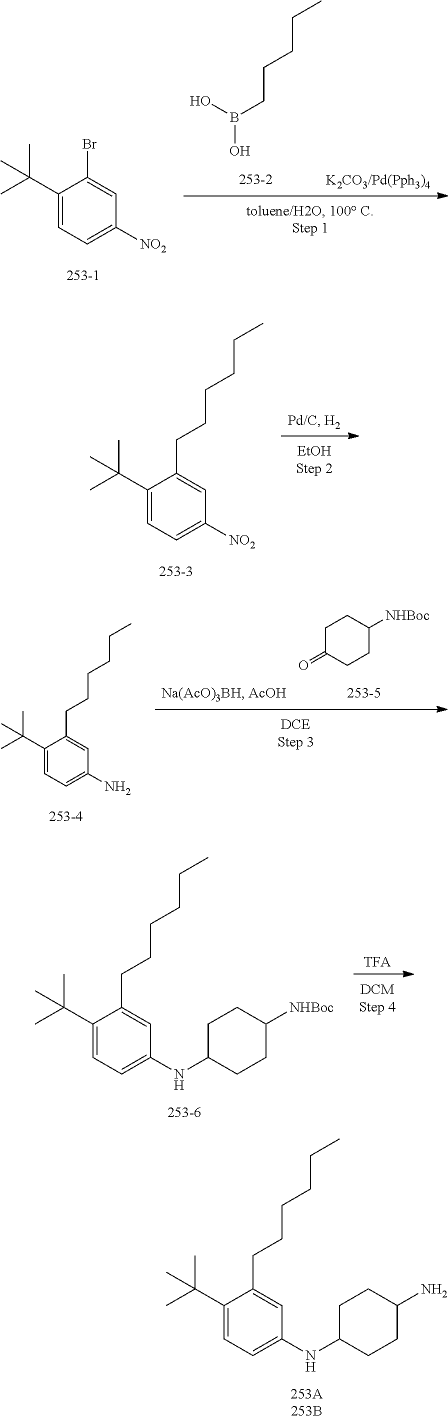

Step 1. Preparation of 1-(tert-butyl)-2-hexyl-4-nitrobenzene (253-3)

The title compound 253-3 (150 mg) was prepared in a total yield of 46.2% as a light-yellow oil from 2-bromo-1-(tert-butyl)-4-nitrobenzene (193 mg, 0.74 mmol), pentylboronic acid (593 mg, 4.5 mmol), K₂CO₃ (153 mg, 1.11 mmol) and Pd(PPh₃)₄ (177 mg, 0.15 mmol) according to the procedure for 208-1.

Step 2. Preparation of 4-(tert-butyl)-3-hexylaniline (253-4)

The title compound 253-4 (50 mg) was prepared in a total yield of 63.1% as a yellow solid from 1-(tert-butyl)-2-hexyl-4-nitrobenzene (150 mg, 0.57 mmol) and 10% Pd/C (3.6 mg, 3.4 μmol) according to the procedure for 208-2. Mass (m/z): 234.3 [M+H]⁺.

Step 3. Preparation of tert-butyl (4-((4-(tert-butyl)-3-hexylphenyl)amino)cyclohexyl)carbamate (253-6)

The title compound 253-6 (35 mg) was prepared in a total yield of 38.8% as a yellow solid from 4-(tert-butyl)-3-hexylaniline (50 mg, 0.21 mmol), tert-butyl (4-oxocyclohexyl)carbamate (89 mg, 0.42 mmol) and Na(AcO)₃BH (89 mg, 0.42 mmol) according to the procedure for 24-1. Mass (m/z): 431.4 [M+H]⁺.

Step 4. Preparation of N1-(4-(tert-butyl)-3-hexylphenyl)cyclohexane-1,4-diamine (253)

The title compound 253A and 253B were prepared according to the procedure for compound 24. The crude residue was purified by preparative TLC (H₂O/MeOH/DCM=0.1/1/5) to afford compound 253A (Rf value=0.40) in 57.4% yield as a white solid and 253B (Rf value=0.36) in 15.3% yield as a white solid. 253A: ¹H NMR (400 MHZ, Methanol-d₄) δ 7.08 (d, J=8.8 Hz, 1H), 6.49 (d, J=2.8 Hz, 1H), 6.39 (dd, J=8.6, 2.8 Hz, 1H), 3.56-3.49 (m, 1H), 3.24-3.15 (m, 1H), 2.74-2.67 (m, 2H), 1.88-1.71 (m, 8H), 1.61-1.52 (m, 2H), 1.46-1.36 (m, 2H), 1.36-1.31 (m, 13H), 0.94-0.86 (m, 3H). Mass (m/z): 331.3 [M+H]⁺. 253B: ¹H NMR (400 MHz, Methanol-d₄) δ 7.08 (d, J=8.6 Hz, 1H), 6.49 (d, J=2.8 Hz, 1H), 6.40 (dd, J=8.6, 2.7 Hz, 1H), 3.24-3.17 (m, 1H), 3.11-3.04 (m, 1H), 2.74-2.67 (m, 2H), 2.17-2.10 (m, 2H), 2.09-1.99 (m, 2H), 1.57-1.26 (m, 21H), 0.93-0.85 (m, 3H). Mass (m/z): 331.3 [M+H]⁺.

### Compound 254

### N1-(4-(tert-butyl)-3-(4-methoxybutoxy) phenyl)cyclohexane-1,4-diamine

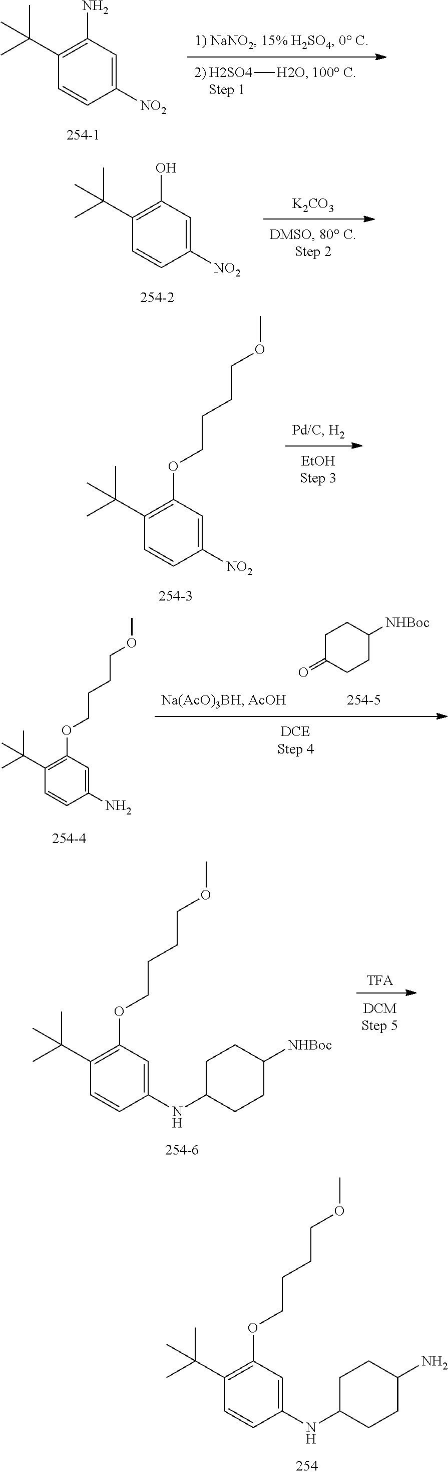

Step 1. Preparation of 2-(tert-butyl)-5-nitrophenol (254-2)

To a mixture of 2-tert-butyl-5-nitroaniline (582 g, 3.0 mmol) in 10 mL of 15% H₂SO₄ was added dropwise a solution of NaNO₂ (217 mg, 3.15 mmol) in water (3 mL) at 0° C. The resulting mixture was stirred at 0-5° C. for 20 min. Then the solution was added dropwise to a solution of 5 mL of H₂SO₄—H₂O (V/V=1/2) stirred at 100° C. The resulting mixture was stirred at 100° C. for 20 min. After cooling to rt, and extracted by DCM (20 mL×3), the combined organic layers were washed with water (20 mL), dried over Na₂SO₄ and concentrated. The residue was purified by prep-TLC (DCM/PE=1/3) to afford the title compound as a yellow oil (300 mg, 51.5%). Mass (m/z): 194.0 [M−H]⁺.

Step 2. Preparation of 1-(tert-butyl)-2-(4-methoxybutoxy)-4-nitrobenzene (254-3)

To a mixture of 2-(tert-butyl)-5-nitrophenol (150 mg, 0.77 mmol), KI (12.8 mg, 77 μmmol) and K₂CO₃ (212 mg, 1.54 mmol) in DMSO (2.0 mL) was added 1-bromo-4-methoxybutane (190 mg, 1.15 mmol). Then the mixture was stirred overnight at 80° C. After cooling to rt, 5 mL was added, and extracted by DCM (10 mL×3). The combined organic layers were washed with water (10 mL×3), dried over Na₂SO₄ and concentrated to afford the title compound as a crude yellow oil (216 mg, 100%).

Step 3. Preparation of 4-(tert-butyl)-3-(4-methoxybutoxy) aniline (254-4)

The title compound 254-4 (193 mg) was prepared in a total yield of 100% as a yellow solid from 1-(tert-butyl)-2-(4-methoxybutoxy)-4-nitrobenzene (216 mg, 0.77 mmol) and 10% Pd/C (81.6 mg, 77 μmol) according to the procedure for 208-2. Mass (m/z): 252.4 [M+H]⁺.

Step 4. Preparation of N1-(4-(tert-butyl)-3-(4-methoxybutoxy) phenyl)cyclohexane-1,4-diamine (254-6)

The title compound 254-6 (62 mg) was prepared in a total yield of 18.0% as a yellow solid from 4-(tert-butyl)-3-(4-methoxybutoxy) aniline (193 mg, 0.77 mmol), tert-butyl (4-oxocyclohexyl)carbamate (328 mg, 1.54 mmol) and Na(AcO)3BH (326 mg, 1.54 mmol) according to the procedure for 24-1. Mass (m/z): 449.4 [M+H]⁺.

Step 5. Preparation of N1-(4-(tert-butyl)-3-(4-methoxybutoxy) phenyl)cyclohexane-1,4-diamine (254)

The title compound 254 (32.2 mg) was prepared in a total yield of 66.1% as a white solid with 1:1 mixture by ¹H NMR from N1-(4-(tert-butyl)-3-(4-methoxybutoxy) phenyl)cyclohexane-1,4-diamine (62 mg, 0.14 mmol) and TFA (2.0 mL) according to the procedure for 24. ¹H NMR (400 MHZ, Methanol-d₄) δ 6.99 (dd, J=10.4, 8.4 Hz, 1H), 6.30 (t, J=2.6 Hz, 1H), 6.20 (dd, J=8.4, 2.3 Hz, 0.5H), 6.15 (dd, J=8.4, 2.3 Hz, 0.5H), 3.93 (t, J=6.0 Hz, 2H), 3.56-3.49 (m, 0.5H), 3.46 (t, J=6.2 Hz, 2H), 3.33 (s, 3H), 3.26-3.19 (m, 1H), 3.12-3.04 (m, 0.5H), 2.19-2.11 (m, 1H), 2.10-2.04 (m, 0.5H), 1.95-1.65 (m, 8H), 1.56-1.45 (m, 1H), 1.37-1.12 (m, 11H). Mass (m/z): 349.3 [M+H]⁺.

### Compound 255

### 4-((4-(tert-butyl)phenyl)amino)cyclohexane-1,2-diol

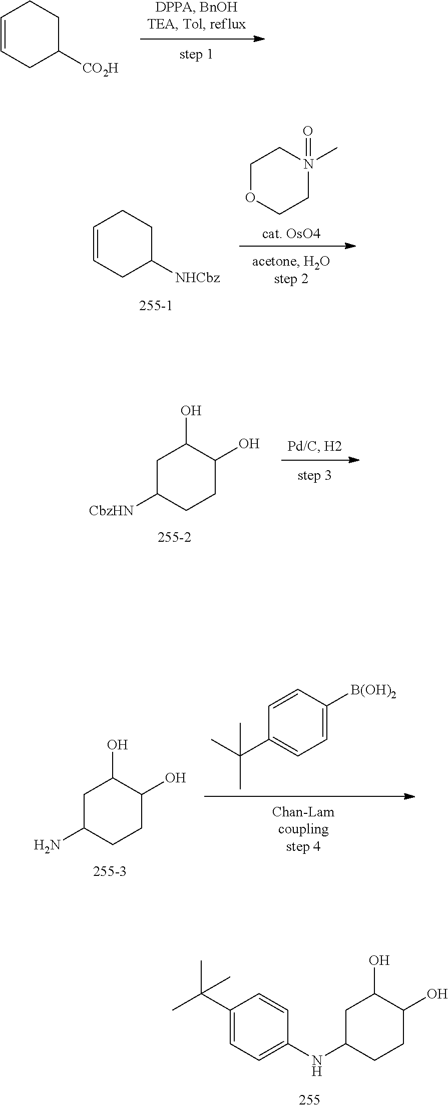

Step 1. Preparation of benzyl cyclohex-3-en-1-ylcarbamate (255-1)

A mixture of cyclohex-3-ene-1-carboxylic acid (1 g, 7.93 mmol), TEA (882 mg, 8.72 mmol) and DPPA (2.18 g, 7.93 mmol) in toluene (40 mL) was refluxed for 2 hours. After BnOH (857 mg, 7.93 mmol), the mixture was refluxed for 10 hours. The mixture was evaporated in vacuo and the obtained residue was diluted with ethyl acetate. The organic layer was washed successively with 1N hydrochloric acid, an aqueous saturated sodium hydrogen carbonate solution and brine. The organic phases were combined, dried over Na₂SO₄, filtered and concentrated to afford the product (1.56 g, 85%) as a yellow solid. Mass (m/z): 232.1 [M+H]⁺.

Step 2. Preparation of benzyl (3,4-dihydroxycyclohexyl)carbamate (255-2)

To a solution of benzyl (3,4-dihydroxycyclohexyl)carbamate (1.56 g, 6.74 mmol) in a mixture of tetrahydrofuran (40 mL) and water (5 mL) was added 4-methylmorpholine 4-oxide (1.19 g, 10.1 mmol) and OsO₄ (500 mg, 1.97 mmol). The mixture was stirred at ambient temperature for 30 minutes. The resulting mixture was quenched with Na₂S₂O₃ aqueous and the obtained residue was diluted with ethyl acetate. The organic phases were combined, dried over Na₂SO₄, filtered and concentrated to afford the product (1.1 g, 62%) as a white solid. Mass (m/z): 266.1 [M+H]⁺.

Step 3. Preparation of 4-aminocyclohexane-1,2-diol (255-3)

A mixture of benzyl (3,4-dihydroxycyclohexyl)carbamate (700 mg, 2.64 mmol) and Pd/C (281 mg, 0.26 mmol) in ethanol (10 mL) was stirred at ambient temperature under hydrogen atmosphere for overnight. The catalyst was removed by filtration, and the filtrate was evaporated in vacuo. The target product (288 mg, 83%) was obtained as a white solid. Mass (m/z): 132.1 [M+H]⁺.

Step 4. Preparation of 4-((4-(tert-butyl)phenyl)amino)cyclohexane-1,2-diol (255)

To a solution of 4-aminocyclohexane-1,2-diol (288 mg, 2.20 mmol), (4-(tert-butyl)phenyl) boronic acid (391 mg, 2.20 mmol) and TEA (1.11 g, 11 mmol) in CH₂Cl₂ (20 ml) was added Cu(OAc)₂ (877 mg, 4.39 mmol). Then the mixture was stirred for overnight at rt. The reaction was filtered through celite and the filtrate was evaporated in vacuo. The residue was purified by prep-TLC to afford the desired product 255 (11.4 mg, 4%) as a yellow solid. ¹H NMR (400 MHZ, CD₃OD) δ 7.15 (d, J=8.8 Hz, 2H), 7.15 (d, J=8.8 Hz, 2H), 4.01-3.94 (m, 1H), 3.70-3.56 (m, 2H), 2.21-2.12 (m, 1H), 2.06-1.97 (m, 1H), 1.86-1.75 (m, 1H), 1.75-1.65 (m, 1H), 1.44-1.35 (m, 1H), 1.26 (s, 9H). Mass (m/z): 263.9 [M+H]⁺.

### Compound 256

### N1-(4-(tert-butyl)phenyl)-N2-(2-ethoxyethyl)-N2-(4-methoxybutyl) ethane-1,2-diamine

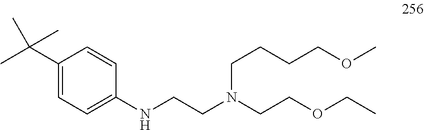 [SMILES: CCOCCN(CCCCOC)CCNc1ccc(C(C)(C)C)cc1]

The title compound 256 (44 mg, 33%) as a white solid was prepared from N-(4-(tert-butyl)phenyl)-2-((2-ethoxyethyl)(4-methoxybutyl)amino)acetamide (139 mg, 0.38 mmol), and BH₃-THF (20 mL) according to the procedure for 105. ¹H NMR (400 MHZ, CD₃OD) δ 7.22 (d, J=8.7 Hz, 2H), 6.67 (dd, J=8.6, 2.0 Hz, 2H), 3.70 (s, 2H), 3.48 (q, J=7.0 Hz, 4H), 3.39 (dt, J=4.3, 3.7 Hz, 6H), 3.32 (s, 3H), 3.27-3.18 (m, 2H), 1.85-1.74 (m, 2H), 1.64-1.56 (m, 3H), 1.28-1.25 (m, 9H), 1.15 (t, J=7.0 Hz, 3H). Mass (m/z): 350.9 [M+H]⁺.

### Compound 257

### N-(4-cyclohexylphenyl)-1-ethylpiperidin-4-amine

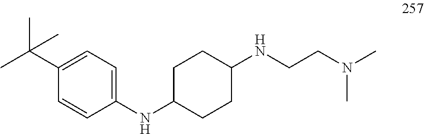 [SMILES: CN(C)CCNC1CCC(Nc2ccc(C(C)(C)C)cc2)CC1]

The title compound 257 (17.2 mg, 13.2%) as a yellow solid was prepared from 4-((4-(tert-butyl)phenyl)amino)cyclohexan-1-one (100 mg, 0.41 mmol), MeOH (5 ml), N1, N1-dimethylethane-1,2-diamine (36 mg, 0.41 mmol) and acetic acid (2.46 mg, 0.041 mmol) according to the procedure for 5. ¹H NMR (400 MHZ, CDCl₃) δ 8.49 (s, 1H), 7.17 (d, J=8.0 Hz, 2H), 6.52 (d, J=8.0 Hz, 2H), 3.26-2.95 (m, 5H), 2.52 (s, 6H), 2.22 (m, 4H), 1.62 (s, 2H), 1.25 (s, 9H), 1.21-1.04 (m, 2H). Mass (m/z): 318.3 [M+H]⁺.

### Compound 258

### 1-(4-((4-(tert-butyl)phenyl)amino)cyclohexyl)-N,N,N-trimethylmethanaminium

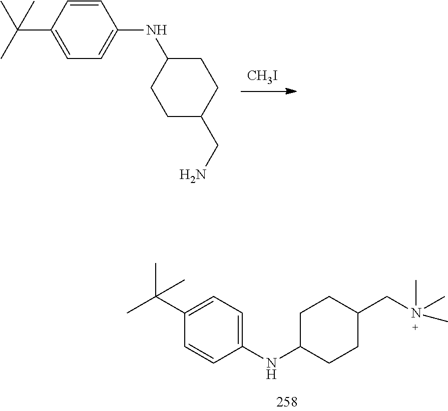

To a solution of N-(4-(aminomethyl)cyclohexyl)-4-(tert-butyl)aniline (120 mg, 0.46 mmol) in MeOH (5 mL) was added K₂CO₃ (180 mg, 1.30 mmol) and iodomethane (327 mg, 2.3 mmol). Then the mixture was stirred at 25° C. for 12 h. Quenched with water (20 mL), the reaction was extracted by EA (10 mL) for 3 times. The combined organic layers were dried over sodium sulfate, concentrated under vacuum and the residue was purified by prep-HPLC (column-Xbridge-C_{18 150×21.2} mm, 5 μm; Mobile phase: ACN-H₂O (0.1% FA), 40%-60%) to afford the desired product 258 (10.1 mg, 7.2%) as a white solid. ¹H NMR (400 MHZ, DMSO-d₆) δ 8.38 (s, 1H), 7.07 (d, J=8.4 Hz, 2H), 6.53 (d, J=8.4 Hz, 2H), 3.37 (s, 1H), 3.24 (s, 2H), 3.07 (s, 9H), 2.07 (s, 1H), 1.67 (d, J=12 Hz, 2H), 1.55 (s, 6H), 1.20 (s, 9H). Mass (m/z): 303.3 [M+H]⁺.

### Compound 259

### N-(4-{[4-(4,4-dimethylcyclohexyl)phenyl]amino}cyclohexyl)aminosulfonamide

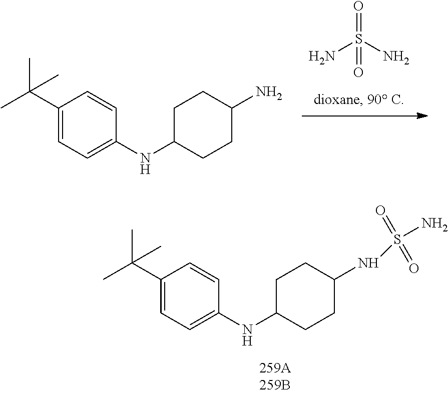 [SMILES: CC(C)(C)c1ccc(NC2CCC(N)CC2)cc1 ; NS(N)(=O)=O ; CC(C)(C)c1ccc(NC2CCC(NS(N)(=O)=O)CC2)cc1]

A solution of N1-(4-(tert-butyl)phenyl)cyclohexane-1,4-diamine (70 mg, 0.28 mmol), and sulfamoylamine (32.8 mg, 0.34 mmol) in 1,4-dioxane (10 mL) was stirred overnight at 90° C. under N₂. After cooling to rt., the reaction was concentrated under vacuum and the residue was purified by prep-HPLC (column-Xbridge-C18 150×21.2 mm, 5 μm; Mobile phase: ACN-H₂O (0.1% FA), 20%-40%) to afford the desired product 295A (19.3 mg, 20.6%) as a white solid and 295B (11.0 mg, 11.7%) as a white solid. 259A: ¹H NMR (400 MHZ, DMSO-d₆) δ 7.06 (d, J=8.6 Hz, 2H), 6.51 (d, J=8.6 Hz, 2H), 6.45 (s, 2H), 6.38 (d, J=5.8 Hz, 1H), 5.11 (br, 1H), 3.57 (br, 1H), 2.54 (br, 1H), 1.76-1.56 (m, 8H), 1.20 (s, 9H). Mass (m/z): 326.2 [M+H]⁺. HPLC: Rt=3.384 min (Column: XBRIDGE 3.5 μm, 2.1*50 mm, Mobile phase: H₂O (0.05% TFA)-ACN (0.05% TFA), ACN from 0 to 60% over 7 minutes, 7-8 min, ACN from 60% to 100%, Flow rate: 0.8 mL/min). 259B: ¹H NMR (400 MHZ, DMSO-d₆) δ 7.40 (br, 2H), 7.17 (br, 2H), 6.57 (d, J=6.7 Hz, 1H), 6.50 (br, 2H), 3.25 (br, 1H), 3.03 (br, 1H), 1.96 (t, J=15.2 Hz, 4H), 1.42 (br, 2H), 1.26 (s, 9H), 1.21-1.18 (m, 2H). Mass (m/z): 326.2 [M+H]⁺. HPLC: Rt=3.273 min (Column: XBRIDGE 3.5 μm, 2.1*50 mm, Mobile phase: H₂O (0.05% TFA)-ACN (0.05% TFA), ACN from 0 to 60% over 7 minutes, 7-8 min, ACN from 60% to 100%, Flow rate: 0.8 mL/min).

### Compound 260

### N-(((1r,4r)-4-aminocyclohexyl)methyl)-4-(tert-butyl)aniline

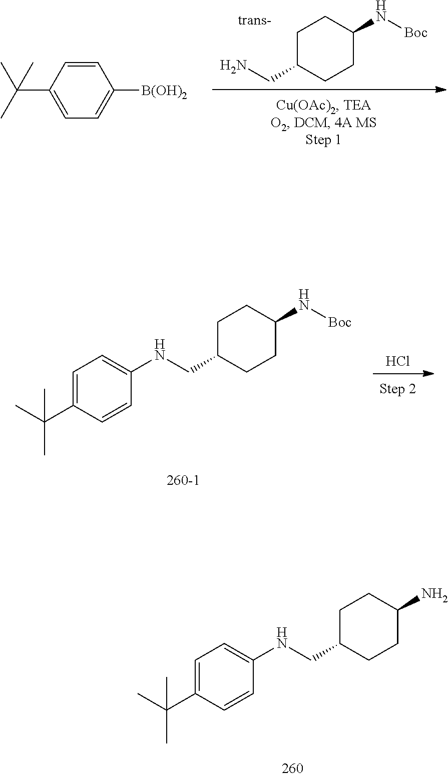 [SMILES: CC(C)(C)c1ccc(B(O)O)cc1 ; CC(C)(C)OC(=O)N[C@H]1CC[C@H](CN)CC1 ; CC(C)(C)OC(=O)N[C@H]1CC[C@H](CNc2ccc(C(C)(C)C)cc2)CC1 ; Cl ; CC(C)(C)c1ccc(NC[C@H]2CC[C@H](N)CC2)cc1]

Step 1. Preparation of tert-butyl ((1r,4r)-4-(((4-(tert-butyl)phenyl)amino)methyl)cyclohexyl)carbamate (260-1)

A 100-mL round-bottom flask was charged with tert-butyl ((1r,4r)-4-(aminomethyl)cyclohexyl)carbamate (232 mg, 1.01 mmol, 1.1 eq), (4-(tert-butyl)phenyl) boronic acid (150 mg, 0.84 mmol, 1.00 eq), Cu (OAc) 2 (306 mg, 1.7 mmol, and 2.00 eq) and TEA (426 mg, 4.2 mmol, 5.00 eq) and 4 Å MS (1 g). The reaction was stirred at R.T. under O₂ atmosphere for 18 hours. The solids were filtered and solvent was removed under vacuum. The residue was purified by Flash Chromatography to give 260-1 (0.2 g, 65.6% yield) as a yellow solid. MS (m/z) 361.3 [M+H]⁺.

Step 2. Preparation of N-(((1r,4r)-4-aminocyclohexyl)methyl)-4-(tert-butyl)aniline (260)

260-1 (200 mg, 0.28 mmol) and HCl in 1,4-dioxane (10 mL, 4N) were placed in a flask stirred at 25° C. for 16 hrs. Excess 1,4-dioxane was distilled under vacuum and the residue was purified by Flash Chromatography to afford 260 (68.2 mg 47.3%) as a white solid. ¹H NMR (400 MHZ, DMSO-d₆) δ 7.06 (d, J=8.6 Hz, 2H), 6.46 (d, J=8.6 Hz, 2H), 5.36 (t, J=5.5 Hz, 1H), 2.79 (t, J=6.2 Hz, 2H), 1.76 (s, 4H), 1.42 (s, 1H), 1.20 (s, 9H), 0.94 (d, J=8.4 Hz, 4H). MS (m/z) 261.3 [M+H]⁺.

### Compound 261

### 4-((4-(4,4-dimethylcyclohexyl)phenyl)amino)tetrahydro-2H-thiopyran 1,1-dioxide

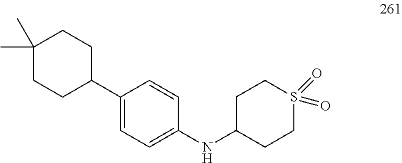 [SMILES: CC1(C)CCC(c2ccc(NC3CCS(=O)(=O)CC3)cc2)CC1]

The desired product 261 (5.4 mg, 4.5%) as a white solid was prepared from 4-(4,4-dimethylcyclohexyl)aniline (70 mg, 0.34 mmol), tetrahydro-4H-thiopyran-4-one 1,1-dioxide (61.2 mg, 0.41 mmol), borane-2-picoline complex (55.2 mg, 0.52 mmol), CH₃COOH (1 mL) and H₂O (9 mL) according to the procedure for 90. ¹H NMR (400 MHZ, DMSO-d₆) δ 6.96 (d, J=8.3 Hz, 2H), 6.53 (d, J=8.4 Hz, 2H), 5.46 (d, J=8.7 Hz, 1H), 3.60 (d, J=8.7 Hz, 1H), 3.23-3.10 (m, 4H), 2.23-2.13 (m, 3H), 1.92-1.83 (m, 2H), 1.59-1.37 (m, 6H), 1.31-1.23 (m, 2H), 0.93 (d, J=8.8 Hz, 6H). Mass (m/z): 336.3 [M+H]⁺.

### Compound 262

### (1s,4s)-N1-(4-(3-ethoxypropyl)phenyl)cyclohexane-1,4-diamine

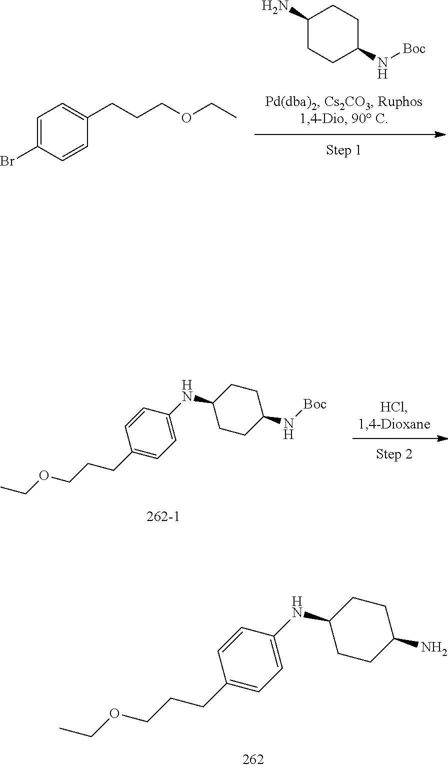 [SMILES: CC(C)(C)OC(=O)N[C@H]1CC[C@@H](N)CC1 ; CCOCCCc1ccc(Br)cc1 ; CCOCCCc1ccc(N[C@H]2CC[C@@H](NC(=O)OC(C)(C)C)CC2)cc1 ; "HCl,1,4-Dioxane" ; CCOCCCc1ccc(N[C@H]2CC[C@@H](N)CC2)cc1]

Step 1. of tert-butyl Preparation ((1s,4s)-4-((4-(3-ethoxypropyl)phenyl)amino)cyclohexyl)carbamate (262-1)

A mixture of 1-bromo-4-(3-ethoxypropyl) benzene (200 mg, 0.82 mmol), tert-butyl ((1s,4s)-4-aminocyclohexyl)carbamate (352.6 mg, 1.65 mmol), Pd₂(dba)₃ (75.3 mg, 0.08 mmol), Ruphos (76.8 mg, 0.16 mmol), Cs₂CO₃ (536.0 mg, 1.65 mmol) in 1,4-dioxane (10 mL) was stirred overnight at 90° C. under N₂ atmosphere. After cooling to rt, the reaction solution was washed with water, and the reaction solution was extracted three times with ethyl acetate (20 mL) and water (20 mL). Organic layers were combined, the solvent was removed under vacuum and the crude was purified through silica gel chromatography (PE:EA=5:1) to give the desired product (300 mg, 58.1%) as an oil. Mass (m/z): 377.3 [M+H]⁺.

Step 2. Preparation of (1s,4s)-N1-(4-(3-ethoxypropyl)phenyl)cyclohexane-1,4-diamine (262)

The mixture of tert-butyl ((1s,4s)-4-((4-(3-ethoxypropyl)phenyl)amino)cyclohexyl)carbamate (300 mg, 0.79 mmol) in 1,4-dioxane (10 mL) and 1,4-dioxane/HCl (10 mL) was stirred for 16 hour at 25° C. After reaction was completed, solvent was removed under vacuum and the residue was purified by prep-HPLC (column-Xbridge-C18 150×21.2 mm, 5 μm; Mobile phase: ACN-H₂O (0.1% FA), 5%-20%) to afford the desired product 262 as a white solid. (23.6 mg, 10.7%). ¹H NMR (400 MHZ, DMSO-d₆) δ 6.88 (d, J=8.3 Hz, 2H), 6.54 (d, J=8.3 Hz, 2H), 5.36 (br, 1H), 3.41-3.35 (m, 3H), 3.31 (t, J=6.5 Hz, 2H), 3.02 (br, 1H), 2.43 (t, J=7.6 Hz, 2H), 1.93-1.53 (m, 10H), 1.10 (t, J=7.0 Hz, 3H). Mass (m/z): 277.3 [M+H]⁺.

### Compound 263

### 4-(tert-butyl)-N-(3-((methylamino)methyl)cyclopentyl)aniline

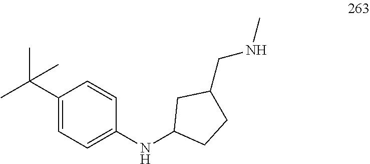 [SMILES: CNCC1CCC(Nc2ccc(C(C)(C)C)cc2)C1]

The title compound 263 (18.8 mg) was prepared in a total yield of 22.0% as a yellow solid from 3-((4-(tert-butyl)phenyl)amino)-N-methylcyclopentane-1-carboxamide (90 mg, 0.328 mmol), LiAlH₄ (49 mg, 1.314 mmol) and THF (10 mL) according to the procedure for 23. ¹H NMR (400 MHZ, DMSO-d₆) δ 7.09-6.96 (m, 2H), 6.45 (d, J=8.2 Hz, 2H), 5.63-5.22 (m, 1H), 3.64 (s, 1H), 2.82 (s, 2H), 2.43 (s, 3H), 2.26-2.19 (m, 2H), 1.89-1.69 (m, 3H), 1.47-1.43 (m, 1H), 1.16 (s, 9H). Mass (m/z): 261.3 [M+H]⁺.

### Compound 264

### N-(4-(2-aminoethyl)cyclohexyl)-2,3-dihydro-1H-inden-5-amine

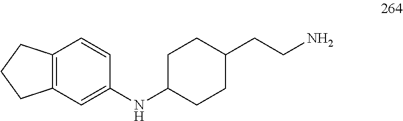 [SMILES: NCCC1CCC(Nc2ccc3c(c2)CCC3)CC1]

The title compound 264 (47.1 mg) was prepared in a total yield of 34.2% as a brown solid from 2-(4-((2,3-dihydro-1H-inden-5-yl)amino)cyclohexyl)acetamide (145 mg, 0.533 mmol), LiAlH₄ (79 mg, 2.132 mmol) and THF (10 mL) according to the procedure for 23. ¹H NMR (400 MHZ, DMSO-d₆) δ 6.90 (dd, J=8.0, 4.2 Hz, 1H), 6.55-6.38 (m, 2H), 5.65-4.98 (m, 1H), 2.71 (dq, J=14.8, 8.8, 7.2 Hz, 6H), 1.93 (p, J=7.2 Hz, 3H), 1.76-1.33 (m, 11H). Mass (m/z): 259.3 [M+H]⁺.

### Compound 265

### N1-(5,5,8,8-tetramethyl-5,6,7,8-tetrahydronaphthalen-2-yl)cyclohexane-1,4-diamine

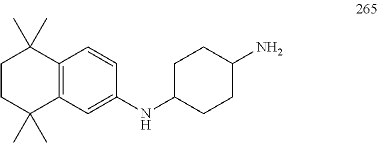 [SMILES: CC1(C)CCC(C)(C)c2cc(NC3CCC(N)CC3)ccc21]

The title compound 265 (21.8 mg) was prepared in a yield of 29.5% as a white powder with 1:0.41 mixture by ¹H NMR from 5,5,8,8-tetramethyl-5,6,7,8-tetrahydronaphthalen-2-amine (50 mg, 0.25 mmol), tert-butyl (4-oxocyclohexyl)carbamate (78 mg, 0.37 mmol) and according to the procedure for 20. ¹H NMR (400 MHZ, DMSO-d₆) δ 8.01 (s, 4H), 7.00 (dd, J=8.5, 4.7 Hz, 1H), 6.51 (d, J=2.5 Hz, 0.40H), 6.45 (d, J=2.5 Hz, 1H), 6.37 (td, J=8.6, 8.0, 2.4 Hz, 1H), 5.14 (d, J=5.4 Hz, 0.41H), 5.08 (d, J=8.0 Hz, 1H), 3.40 (s, 0.41H), 3.17 (d, J=4.9 Hz, 1H), 3.08 (d, J=8.3 Hz, 1H), 2.98 (tt, J=11.5, 3.7 Hz, 1H), 1.99 (td, J=10.4, 5.3 Hz, 4H), 1.85-1.67 (m, 2H), 1.58 (s, 6H), 1.44 (qd, J=13.5, 12.4, 3.8 Hz, 2H), 1.21-1.13 (m, 18H). Mass (m/z): 301.6 [M+H]⁺.

### Compound 266

### 1-(4-((4-(tert-butyl)phenyl)amino)cyclohexyl)urea

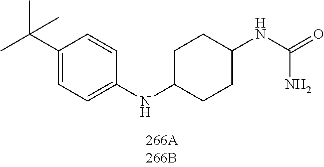 [SMILES: CC(C)(C)c1ccc(NC2CCC(NC(N)=O)CC2)cc1]

The title compounds 266A (26.94 mg) as white solid and 266B (25.46 mg) as a white solid were prepared from 1-N-(4-(tert-butyl)phenyl)cyclohexane-1,4-diamine (100 mg, 0.4 mmol), DCM (7 mL), TMSNCO (46.76 mg, 0.4 mmol), TEA (104.92 mg, 0.81 mmol) and DMAP (9.92 mg, 0.08 mmol) according to the procedure for 178. 266A: ¹HNMR (400 MHZ, CD₃OD) δ 7.15-7.12 (m, 2H), 6.62-6.59 (m, 2H), 3.50-3.37 (m, 1H), 3.19-3.13 (m, 1H), 2.05 (dd, J=7.0, 3.6 Hz, 2H), 1.98-1.92 (m, 2H), 1.25-1.21 (m, 13H). Mass (m/z): 289.9 [M+H]⁺.

HPLC: Rt=4.262 min (Column: XBRIDGE 3.5 μm, 2.1*50 mm, Mobile phase: H₂O (0.05% TFA)-ACN (0.05% TFA), ACN from 0 to 60% over 7 minutes, 7-8 min, ACN from 60% to 100%, Flow rate: 0.8 mL/min). 266B: ¹HNMR (400 MHZ, CD₃OD) δ 7.15-7.12 (m, 2H), 6.62-6.58 (m, 2H), 3.64 (s, 1H), 3.36 (tt, J=7.0, 3.6 Hz, 1H), 1.80-1.72 (m, 2H), 1.65 (dd, J=10.8, 5.4 Hz, 4H), 1.61-1.53 (m, 2H), 1.23 (s, 9H). Mass (m/z): 289.9 [M+H]⁺. HPLC: Rt=4.328 min (Column: XBRIDGE 3.5 μm, 2.1*50 mm, Mobile phase: H₂O (0.05% TFA)-ACN (0.05% TFA), ACN from 0 to 60% over 7 minutes, 7-8 min, ACN from 60% to 100%, Flow rate: 0.8 mL/min).

### Compound 267

### 1-N-(3-aminopropyl)-4-N-(4-(tert-butyl)phenyl)cyclohexane-1,4-diamine

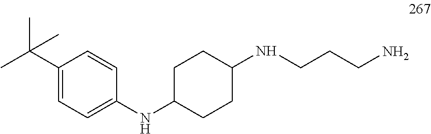 [SMILES: CC(C)(C)c1ccc(NC2CCC(NCCCN)CC2)cc1]

The desired product 267 (24.8 mg) as a white solid was prepared from tert-butyl (3-((4-((4-(tert-butyl)phenyl)amino)cyclohexyl)amino)propyl) carba-mate (100 mg, 0.25 mmol), 1,4-dioxane (5 mL) and HCl/1,4-Dioxane (10 mL) according to the procedure for 224. ¹HNMR (400 MHZ, CD₃OD) δ 7.24-7.17 (m, 2H), 6.76 (t, J=9.2 Hz, 2H), 3.46-3.38 (m, 1H), 3.24-3.19 (m, 01H), 3.17-3.13 (m, 1H), 3.03 (t, J=3.6 Hz, 0.5H), 2.68-2.62 (m, 2H), 2.59 (dd, J=9.6, 5.6 Hz, 0.5H), 1.94-1.88 (m, 1H), 1.82-1.70 (m, 3H), 1.67-1.59 (m, 3H), 1.59-1.26 (m, 3H), 1.25 (s, 9H). Mass (m/z): 304.0 [M+H]⁺.

### Compound 268

### 1-N-(2-aminoethyl)-N4-(4-(tert-butyl)phenyl)cyclohexane-1,4-diamine

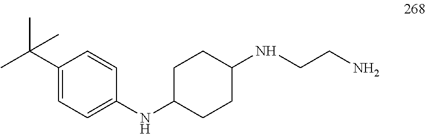 [SMILES: CC(C)(C)c1ccc(NC2CCC(NCCN)CC2)cc1]

The title compound 268 (6 mg) as a white solid was prepared from tert-butyl (2-((4-((4-(tert-butyl)phenyl)amino)cyclohexyl)amino)ethyl) carba-mate (100 mg, 0.25 mmol), 1,4-dioxane (5 mL) and HCl/1,4-dioxane (10 mL) according to the procedure for 224. ¹HNMR (400 MHZ, CD₃OD) δ 7.15 (d, J=8.8 Hz, 2H), 6.62 (d, J=8.8 Hz, 2H), 3.23 (dd, J=6.2, 2.4 Hz, 4H), 3.07 (m, 1H), 2.17 (d, J=11.0 Hz, 4H), 1.56-1.45 (m, 2H), 1.40-1.18 (m, 12H). Mass (m/z): 290.0 [M+H]⁺.

### Compound 269

### 4-cyclohexyl-N-(4-methylcyclohexyl)aniline

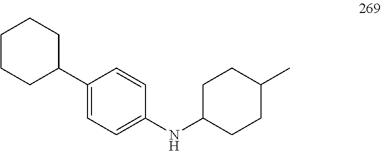 [SMILES: CC1CCC(Nc2ccc(C3CCCCC3)cc2)CC1]

The title compound 269 (10.2 mg) was prepared in a total yield of 74% as a white solid with 1:4 mixture by ¹H NMR from 4-cyclohexylaniline (9 mg, 0.05 mmol) according to the procedure for compound 4. ¹H NMR (400 MHZ, DMSO-d₆) δ 6.89 (d, J=8.4 Hz, 2H), 6.51 (d, J=8.4 Hz, 1.6H), 6.46 (d, J=8.4 Hz, 0.4H), 5.26 (br s, 1H), 3.17 (m, 0.8H), 3.04 (m, 0.2H), 2.28 (m, 1H), 1.80-1.11 (m, 19H), 0.89 (m, 3H). Mass (m/z): 272.1 [M+H]⁺.

### Compound 270

### 1-(aminomethyl)-N4-(4-(tert-butyl)phenyl)cyclohexane-1,4-diamine

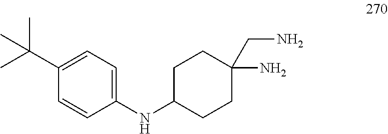 [SMILES: CC(C)(C)c1ccc(NC2CCC(N)(CN)CC2)cc1]

The title compound 270 (6.4 mg) was prepared in a total yield of 46% as a light yellow solid with 1:1 mixture by ¹H NMR from 1-amino-4-((4-(tert-butyl)phenyl)amino)cyclohexane-1-carboxamide (15 mg, 0.05 mmol) according to the procedure for compound 86. ¹H NMR (400 MHZ, DMSO-d₆) δ 8.91 (br s, 4H), 7.12 (d, J=8.4 Hz, 2H), 6.64 (d, J=8.4 Hz, 2H), 5.31 (br s, 1H), 3.27 (s, 2H), 3.20 (m, 0.5H), 3.04 (m, 0.5H), 2.11-1.38 (m, 8H), 1.21 (s, 9H). Mass (m/z): 276.1 [M+H]⁺.

### Compound 271

### N1-(4-(tert-butyl)-3-(hexyloxy) phenyl)cyclohexane-1,4-diamine

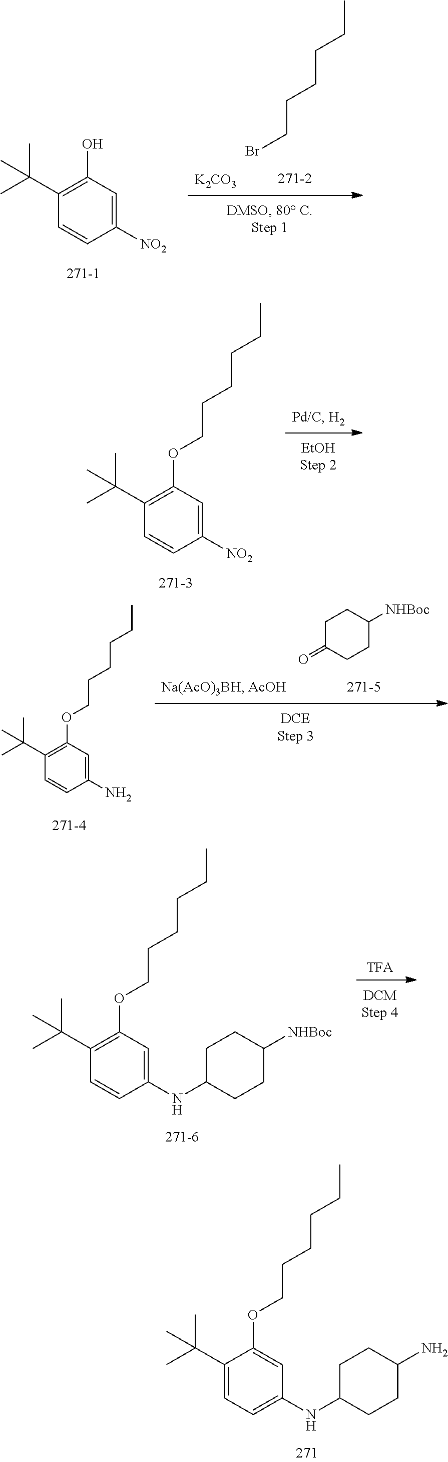

Step 1. Preparation of 1-(tert-butyl)-2-(hexyloxy)-4-nitrobenzene (271-3)

The title compound 271-3 (215 mg) was prepared in a total yield of 100% as a yellow solid from 2-(tert-butyl)-5-nitrophenol (150 mg, 0.77 mmol), 1-bromohexane (165 mg, 1.14 mmol) and K₂CO₃ (212 mg, 1.54 mmol) according to the procedure for 254-3.

Step 2. Preparation of 4-(tert-butyl)-3-(hexyloxy) aniline (271-4)

The title compound 271-4 (60 mg) was prepared in a total yield of 31.3% as a yellow solid from 1-(tert-butyl)-2-(hexyloxy)-4-nitrobenzene (215 mg, 0.77 mmol) and 10% Pd/C (81.6 mg, 77 μmol) according to the procedure for 208-2. Mass (m/z): 250.4 [M+H]⁺.

Step 3. tert-butyl (4-((4-(tert-butyl)-3-(hexyloxy) phenyl)amino)cyclohexyl)carbamate (271-6) The title compound 271-6 (43 mg) was prepared in a total yield of 40.2% as a yellow solid from 4-(tert-butyl)-3-(hexyloxy) aniline (60 mg, 0.24 mmol), tert-butyl (4-oxocyclohexyl)carbamate (102 mg, 0.48 mmol) and Na(AcO)₃BH (102 mg, 0.48 mmol) according to the procedure for 24-1. Mass (m/z): 447.4 [M+H]⁺.

Step 4. N1-(4-(tert-butyl)-3-(hexyloxy) phenyl)cyclohexane-1,4-diamine (271)

The title compound 271 (18.0 mg) was prepared in a total yield of 54.2% as a white solid 1:1 mixture by ¹H NMR from tert-butyl (4-((4-(tert-butyl)-3-(hexyloxy) phenyl)amino)cyclohexyl)carbamate (43 mg, 96 μmol) and TFA (2.0 mL) according to the procedure for 24. ¹H NMR (400 MHZ, Methanol-d₄) δ 7.12-7.01 (m, 0.5H), 6.98 (d, J=8.4 Hz, 0.5H), 6.40-6.10 (m, 2H), 4.06-3.84 (m, 4H), 3.61-3.50 (m, 0.5H), 3.25-3.17 (m, 1H), 3.12-3.02 (m, 0.5H), 2.15 (d, J=12.8 Hz, 1H), 2.06 (d, J=12.6 Hz, 1H), 1.91-1.70 (m, 6H), 1.58-1.44 (m, 4H), 1.38-1.34 (m, 4H), 1.30 (d, J=3.6 Hz, 9H), 0.96-0.85 (m, 3H). Mass (m/z): 347.4 [M+H]⁺.

### Compound 272

N¹-(4-(tert-pentyl)phenyl)cyclopentane-1,3-diamine

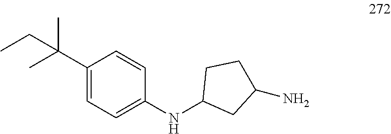 [SMILES: CCC(C)(C)c1ccc(NC2CCC(N)C2)cc1]

The title compound 272 (10.3 mg) was prepared in a total yield of 57.8% as a white solid 1:1 mixture by ¹H NMR from tert-butyl (3-((4-(tert-pentyl)phenyl)amino)cyclopentyl)carbamate (25 mg, 70 μmol) and TFA (2.0 mL) according to the procedure for 24. ¹H NMR (400 MHZ, Methanol-d₄) δ 8.53-8.39 (m, 2H), 8.06-7.95 (m, 2H), 5.38 (p, J=6.0 Hz, 0.5H), 5.24 (p, J=6.5 Hz, 0.5H), 5.11 (p, J=7.2 Hz, 0.5H), 5.05-4.96 (m, 0.5H), 3.98-3.31 (m, 4H), 3.26-2.88 (m, 4H), 2.58 (s, 6H), 2.02 (td, J=7.4, 1.0 Hz, 3H). Mass (m/z): 247.3 [M+H]⁺.

### Compound 273

### N1-(4-(tert-pentyl)phenyl)cyclobutane-1,3-diamine

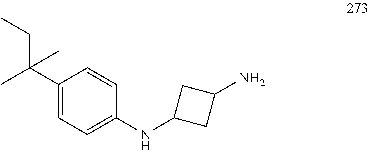 [SMILES: CCC(C)(C)c1ccc(NC2CC(N)C2)cc1]

The title compound 273 (5.0 mg) was prepared in a total yield of 28.4% as a white solid with 3 2 mixture by ¹H NMR from tert-butyl: (3-((4-(tert-pentyl)phenyl)amino)cyclobutyl)carbamate (25 mg, 80 μmol) and TFA (2.0 mL) according to the procedure for 24. ¹H NMR (400 MHZ, Methanol-d₄) δ 7.13-7.02 (m, 2H), 6.57-6.48 (m, 2H), 4.16-4.04 (m, 0.6H), 3.93-3.83 (m, 0.6H), 3.78-3.67 (m, 0.4H), 3.60-3.49 (m, 0.4 H), 2.95-2.78 (m, 0.4H), 2.57-2.39 (m, 1.6H), 2.39-2.27 (m, 1.6H), 2.03-1.85 (m, 0.4H), 1.67-1.45 (m, 2H), 0.62 (t, J=7.4 Hz, 3H). Mass (m/z): 233.3 [M+H]⁺.

### Compound 274

### N-(4-((4-(4,4-dimethylcyclohexyl)phenyl)amino)cyclohexyl) piperazine-2-carboxamide

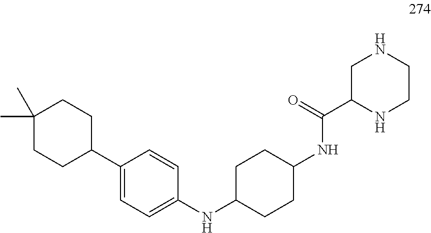 [SMILES: CC1(C)CCC(c2ccc(NC3CCC(NC(=O)C4CNCCN4)CC3)cc2)CC1]

The title compound 274 (28.1 mg) was prepared in a yield of 40.93% as a white powder with 1:1 mixture by ¹H NMR from N1-(4-(4,4-dimethylcyclohexyl)phenyl)cyclohexane-1,4-diamine (50 mg, 0.17 mmol), 1,4-bis(tert-butoxycarbonyl) piperazine-2-carboxylic acid (82 mg, 0.25 mmol) according to the procedure for 1. ¹H NMR (400 MHZ, DMSO-d₆) δ 7.47 (dd, J=24.8, 7.9 Hz, 1H), 6.92 (dd, J=8.4, 3.7 Hz, 2H), 6.48 (dd, J=16.2, 8.1 Hz, 2H), 5.14 (dd, J=25.0, 7.8 Hz, 1H), 3.69 (s, 1H), 3.51 (s, 1H), 3.15-3.00 (m, 2H), 2.87 (d, J=11.9 Hz, 1H), 2.79-2.62 (m, 2H), 2.20 (s, 1H), 1.95 (d, J=12.8 Hz, 1H), 1.82-1.71 (m, 1H), 1.57 (d, J=37.1 Hz, 8H), 1.41 (d, J=13.3 Hz, 3H), 1.28 (q, J=12.8, 11.7 Hz, 4H), 1.15 (d, J=12.4 Hz, 1H), 0.94 (s, 3H), 0.92 (s, 3H). Mass (m/z): 413.5 [M+H]⁺.

### Compound 275

### 1-amino-4-((4-(4,4-dimethylcyclohexyl)phenyl)amino)cyclohexyl)methanol

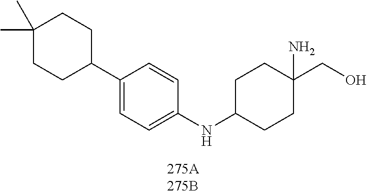 [SMILES: CC1(C)CCC(c2ccc(NC3CCC(N)(CO)CC3)cc2)CC1]

The title compound 275A and 275B were prepared according to the procedure for compound 20. The residue was purified by preparative HPLC (Column: X Select-CSH-Prep 5 μm OBD, 19*150 mm; ACN/water (0.5% TFA)=0%-40%-95%-95%-10%, 0 min-10 min-10.5 min-11.5 min-13.0 min) to afford compound 275A (Rt=5.25 min) in 3.34% yield as a white solid and 275B (Rt=5.53 min) in 13.5% yield as a white solid. 275A: ¹H NMR (400 MHZ, DMSO-d₆) δ 6.91 (d, J=8.2 Hz, 2H), 6.47 (d, J=8.4 Hz, 2H), 5.12 (d, J=8.0 Hz, 1H), 4.67 (s, 1H), 3.19 (s, 1H), 2.21 (dt, J=11.0, 5.4 Hz, 1H), 2.00 (q, J=7.0, 6.3 Hz, 1H), 1.79 (s, 2H), 1.63 (d, J=10.6 Hz, 2H), 1.50 (dd, J=12.9, 9.3 Hz, 4H), 1.42 (d, J=14.0 Hz, 3H), 1.33-1.26 (m, 5H), 0.94 (s, 3H), 0.92 (s, 3H), 0.85 (t, J=6.7 Hz, 1H). Mass (m/z): 331.5 [M+H]⁺. 275B: ¹H NMR (400 MHZ, DMSO-d₆) δ 6.91 (d, J=7.9 Hz, 2H), 6.46 (d, J=8.1 Hz, 2H), 5.07 (s, 1H), 4.58 (s, 1H), 3.11 (s, 2H), 3.03 (s, 1H), 2.20 (s, 1H), 2.01 (s, 2H), 1.69 (s, 3H), 1.51 (s, 6H), 1.35 (s, 3H), 0.94 (s, 3H), 0.92 (s, 3H), 0.85 (s, 1H). Mass (m/z): 331.5 [M+H]⁺.

### Compound 276

### N2-(4-(tert-butyl)phenyl)spiro[3.3]heptane-2, 6-diamine

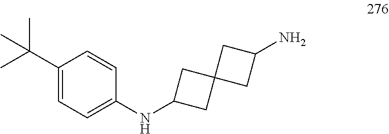 [SMILES: CC(C)(C)c1ccc(NC2CC3(CC(N)C3)C2)cc1]

The title compound 276 (45.2 mg) was prepared in a yield of 34.8% as a white powder with 1:1 mixture by ¹H NMR from 4-(tert-butyl)aniline (75 mg, 0.50 mmol), tert-butyl (6-oxospiro [3.3]heptan-2-yl)carbamate (170 mg, 0.75 mmol) and according to the procedure for 20. ¹H NMR (400 MHZ, DMSO-d₆) δ 8.11 (s, 2H), 7.11-7.00 (m, 2H), 6.45-6.35 (m, 2H), 5.59 (s, 1H), 3.66 (t, J=7.6 Hz, 1H), 3.54 (s, 1H), 2.43-2.29 (m, 2H), 2.22-2.06 (m, 3H), 1.82 (td, J=11.1, 7.7 Hz, 2H), 1.23 (s, 1H), 1.19 (s, 9H). Mass (m/z): 259.2 [M+H]⁺.

### Compound 277

### N2-(4-(tert-butyl)phenyl) octahydropentalene-2,5-diamine

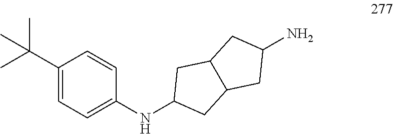 [SMILES: CC(C)(C)c1ccc(NC2CC3CC(N)CC3C2)cc1]

The title compound 277 (24.0 mg) was prepared in a four-step total yield of 13.1% as a white solid with 1:1 mixture by ¹H NMR from 4-(tert-butyl)aniline (100 mg, 0.67 mmol), (3as, 6as)-tetrahydropentalene-2,5 (1H,3H)-dione (278 mg, 2.0 mmol) and NaBH (OAc) 3 (284 mg, 1.34 mmol) according to the procedure for 20. ¹H NMR (400 MHZ, DMSO-d₆) δ 8.06 (d, J=18.4 Hz, 3H), 7.07 (d, J=8.1 Hz, 2H), 6.53 (s, 2H), 5.42 (s, 1H), 3.66 (d, J=6.6 Hz, 1H), 3.51 (s, 1H), 2.36 (q, J=8.2, 7.5 Hz, 1H), 2.29-2.14 (m, 3H), 1.74 (dd, J=12.8, 7.1 Hz, 1H), 1.46-1.33 (m, 2H), 1.19 (s, 9H). Mass (m/z): 273.1 [M+H]⁺.

### Compound 278

### 1-methyl-N4-(4-propylphenyl)cyclohexane-1,4-diamine

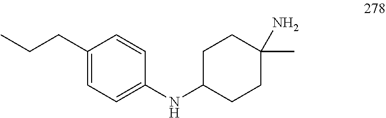 [SMILES: CCCc1ccc(NC2CCC(C)(N)CC2)cc1]

The title compound 278 (60.6 mg) was prepared in a yield of 66.5% as a white powder with 1:1 mixture by ¹H NMR from 4-propylaniline (50 mg, 0.37 mmol), tert-butyl (1-methyl-4-oxocyclohexyl)carbamate (100 mg, 0.44 mmol) and according to the procedure for 20. ¹H NMR (400 MHZ, DMSO-d₆) δ 8.06 (d, J=26.1 Hz, 3H), 6.87 (d, J=7.9 Hz, 2H), 6.51 (d, J=8.3 Hz, 2H), 5.12 (d, J=50.3 Hz, 1H), 3.12 (s, 1H), 2.37 (t, J=7.5 Hz, 2H), 1.88 (td, J=11.0, 9.5, 5.7 Hz, 2H), 1.82-1.70 (m, 2H), 1.65 (td, J=12.6, 3.5 Hz, 1H), 1.55 (d, J=9.9 Hz, 2H), 1.49 (q, J=7.4 Hz, 2H), 1.27 (d, J=7.5 Hz, 4H), 0.85 (t, J=7.3 Hz, 3H). Mass (m/z): 247.3 [M+H]⁺.

### Compound 279

### (2R, 3S)-2-amino-N-(4-((4-(4,4-dimethylcyclohexyl)phenyl)amino)cyclohexyl)-3-hydroxybutana mide

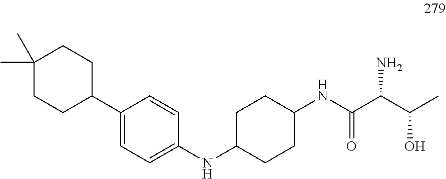 [SMILES: C[C@H](O)[C@@H](N)C(=O)NC1CCC(Nc2ccc(C3CCC(C)(C)CC3)cc2)CC1]

The title compound 279 (9.1 mg) was prepared in a yield of 36.37% as a white powder with 1:1 mixture by ¹H NMR from N1-(4-(4,4-dimethylcyclohexyl)phenyl)cyclohexane-1,4-diamine (100 mg, 0.33 mmol), (tert-butoxycarbonyl)-D-threonine (109 mg, 0.50 mmol) according to the procedure for 1. ¹H NMR (400 MHZ, DMSO-d₆) δ 8.26 (s, 3H), 6.94 (s, 2H), 6.60 (s, 2H), 4.07 (s, 2H), 2.75 (s, 1H), 2.61 (s, 2H), 2.28 (s, 3H), 1.96 (s, 1H), 1.83 (s, 2H), 1.77-1.35 (m, 8H), 1.20 (d, J=26.6 Hz, 4H), 1.02 (d, J=11.7 Hz, 1H), 0.88 (s, 3H), 0.86 (s, 3H). Mass (m/z): 402.3 [M+H]

### Compound 280

### N1-(4-(4-(trifluoromethyl)cyclohexyl)phenyl)cyclohexane-1,4-diamine

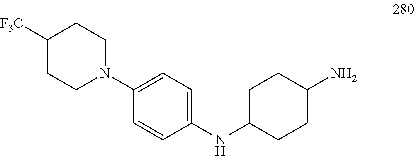 [SMILES: NC1CCC(Nc2ccc(N3CCC(C(F)(F)F)CC3)cc2)CC1]

The title compound 280 (53.4 mg) was prepared in a total yield of 91.4% as a white solid with 1:2 mixture by NMR from tert-butyl (4-((4-(4-(trifluoromethyl)cyclohexyl)phenyl)amino)cyclohexyl)carbamate (75 mg, 0.17 mmol), TFA (1 mL), and DCM (10 mL) according to the procedure for 24. ¹H NMR (400 MHZ, DMSO-d₆) δ 7.00-6.87 (m, 2H), 6.63-6.43 (m, 2H), 5.29 (d, J=26.8 Hz, 1H), 3.00 (d, J=55.2 Hz, 2H), 2.59 (s, 1H), 2.44 (t, J=6.8 Hz, 1H), 2.06-1.90 (m, 3H), 1.73 (dq, J=23.2, 13.6, 9.6 Hz, 10H), 1.51-1.35 (m, 2H), 1.20-1.10 (m, 1H). Mass (m/z): 341.3 [M+H]⁺.

### Compound 281

### N1-(4-ethyl-3,5-dimethylphenyl)cyclohexane-1,4-diamine

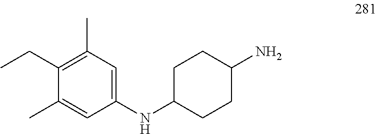 [SMILES: CCc1c(C)cc(NC2CCC(N)CC2)cc1C]

The title compound 281 (66.8 mg) was prepared in a total yield of 91.2% as a yellow solid with mixture by ¹H NMR from tert-butyl 1:2 (4-((4-ethyl-3,5-dimethylphenyl)amino)cyclohexyl)carbamate (103 mg, 0.298 mmol), TFA (1 mL), and DCM (10 mL) according to the procedure for 24. ¹H NMR (400 MHZ, DMSO-d₆) δ 6.24 (d, J=16.8 Hz, 2H), 3.13-2.88 (m, 2H), 2.44 (q, J=7.6 Hz, 2H), 2.15 (s, 6H), 2.12 (d, J=1.8 Hz, 1H), 1.98 (d, J=11.2 Hz, 2H), 1.76 (d, J=14.8 Hz, 3H), 1.63-1.53 (m, 1H), 1.49 (dd, J=12.8, 9.2 Hz, 1H), 0.98 (t, J=7.2 Hz, 3H). Mass (m/z): 247.3 [M+H]⁺.

### Compound 282

### N-(4-(aminomethyl)cyclohexyl)-3,4-dimethylaniline

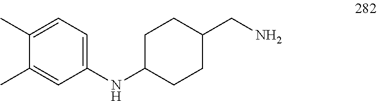 [SMILES: Cc1ccc(NC2CCC(CN)CC2)cc1C]

The title compound 282 (64.1 mg) was prepared in a total yield of 93.4% as a yellow solid with 1:2 mixture by ¹H NMR from tert-butyl ((4-((3,4-dimethylphenyl)amino)cyclohexyl)methyl)carbamate (98 mg, 0.295 mmol), TFA (1 mL), and DCM (10 mL) according to the procedure for 24. ¹H NMR (400 MHZ, DMSO-d₆) δ 6.79 (dd, J=8.4, 2.8 Hz, 1H), 6.50-6.24 (m, 2H), 5.05 (dd, J=30.4, 8.0 Hz, 1H), 3.48-2.99 (m, 1H), 2.64 (dd, J=23.6, 6.8 Hz, 2H), 2.09 (d, J=2.0 Hz, 3H), 2.04 (s, 3H), 2.00-1.92 (m, 1H), 1.83 (dd, J=8.8, 4.8 Hz, 1H), 1.70 (d, J=9.6 Hz, 1H), 1.64-1.58 (m, 1H), 1.49 (pd, J=14.0, 10.8, 4.0 Hz, 4H). Mass (m/z): 233.3 [M+H]⁺.

### Compound 283

### N-(4-(aminomethyl)cyclohexyl)-4-isopropylaniline

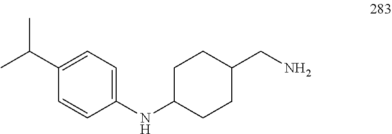 [SMILES: CC(C)c1ccc(NC2CCC(CN)CC2)cc1]

The title compound 283 (52.7 mg) was prepared in a total yield of 70.2% as a yellow solid with 1:2 mixture by ¹H NMR from tert-butyl ((4-((4-isopropylphenyl)amino)cyclohexyl)methyl)carbamate (105 mg, 0.303 mmol), TFA (1 mL), and DCM (10 mL) according to the procedure for 24. ¹H NMR (400 MHZ, DMSO-d₆) δ 7.02-6.88 (m, 2H), 6.60 (d, J=9.7 Hz, 2H), 3.51-3.04 (m, 2H), 2.75-2.59 (m, 3H), 2.03-1.42 (m, 8H), 1.13 (d, J=6.8 Hz, 6H). Mass (m/z): 247.3 [M+H]⁺.

### Compound 284

### N-(4-(aminomethyl)cyclohexyl)-4-isopropylaniline

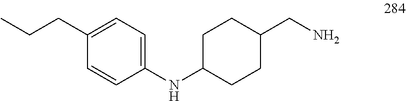 [SMILES: CCCc1ccc(NC2CCC(CN)CC2)cc1]

The title compound 284 (20.7 mg) was prepared in a total yield of 40.9% as a yellow solid with 1:2 mixture by ¹H NMR from tert-butyl ((4-((4-propylphenyl)amino)cyclohexyl)methyl)carbamate (85 mg, 0.246 mmol), TFA (1 mL), and DCM (10 mL) according to the procedure for 24. ¹H NMR (400 MHz, DMSO-d₆) δ 6.86 (dd, J=8.4, 2.8 Hz, 2H), 6.50 (dd, J=27.6, 8.0 Hz, 2H), 5.19 (dd, J=26.4, 8.0 Hz, 1H), 3.43 (s, 1H), 3.07 (s, 1H), 2.66 (dd, J=22.0, 6.8 Hz, 2H), 2.36 (t, J=7.6 Hz, 2H), 2.02-1.77 (m, 2H), 1.53 (tdt, J=25.2, 15.6, 8.8 Hz, 8H), 0.86 (t, J=7.2 Hz, 3H). Mass (m/z): 247.3 [M+H]⁺.

### Compound 285

### 1-methyl-N4-(5,6,7,8-tetrahydronaphthalen-2-yl)cyclohexane-1,4-diamine

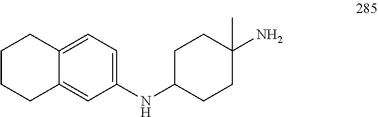 [SMILES: CC1(N)CCC(Nc2ccc3c(c2)CCCC3)CC1]

The title compound 285 (66.8 mg) was prepared in a total yield of 91% as a white solid with 1:2 mixture by ¹H NMR from tert-butyl (1-methyl-4-((5,6,7,8-tetrahydronaphthalen-2-yl)amino)cyclohexyl)carbamate (101 mg, 0.282 mmol), TFA (1 mL), and DCM (10 mL) according to the procedure for 24. ¹H NMR (400 MHZ, DMSO-d₆) § 6.74 (dd, J=8.4, 3.2 Hz, 1H), 6.47-6.22 (m, 2H), 3.31-3.04 (m, 1H), 2.62-2.53 (m, 4H), 2.00-1.83 (m, 2H), 1.81-1.46 (m, 10H), 1.30 (d, J=7.2 Hz, 3H). Mass (m/z): 259.3 [M+H]⁺.

### Compound 286

### N-(4-(aminomethyl)cyclohexyl)-5,6,7,8-tetrahydronaphthalen-2-amine

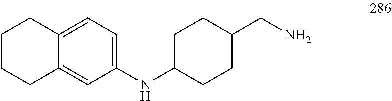 [SMILES: NCC1CCC(Nc2ccc3c(c2)CCCC3)CC1]

The title compound 286 (33.5 mg) was prepared in a total yield of 50% as a yellow solid with 1:2 mixture 1H by NMR from tert-butyl ((4-((5,6,7,8-tetrahydronaphthalen-2-yl)amino)cyclohexyl)methyl)carbamate (93 mg, 0.26 mmol), TFA (1 mL), and DCM (10 mL) according to the procedure for 24. ¹H NMR (400 MHZ, DMSO-d₆) δ 6.72 (dd, J=8.4, 3.2 Hz, 1H), 6.45-6.14 (m, 2H), 5.03 (dd, J=27.2, 8.4 Hz, 1H), 3.51-2.98 (m, 1H), 2.64 (d, J=6.8 Hz, 1H), 2.61-2.51 (m, 5H), 1.97 (d, J=9.6 Hz, 1H), 1.80 (dd, J=13.2, 8.0 Hz, 1H), 1.66 (p, J=2.8 Hz, 5H), 1.56-1.35 (m, 4H), 1.04 (q, J=11.2 Hz, 1H). Mass (m/z): 259.3 [M+H]⁺.

### Compound 287

### N-(4-(3-aminopropyl)cyclohexyl)-4-(tert-butyl)aniline

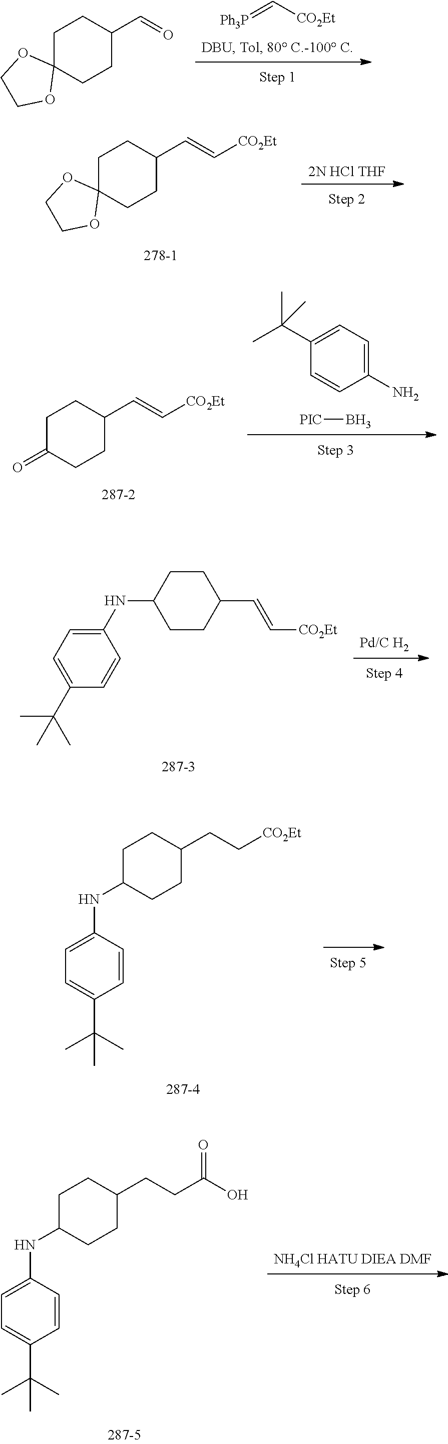

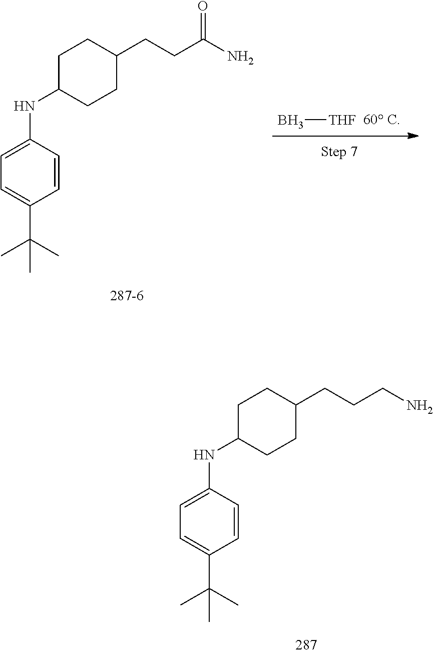

Step 1. Preparation of ethyl (E)-3-(1,4-dioxaspiro[4.5]decan-8-yl)acrylate (287-1)

A solution of 1,4-dioxaspiro[4.5]decane-8-carbaldehyde (1 g, 5.8 mmol), DBU (1.07 g, 7.1 mmol) and ethyl 2-(triphenyl-15-phosphaneylidene) acetate (3 g, 8.8 mmol) in toluene (20 mL) was stirred at 100° C. under N₂ atmosphere for 18 hours. The solid was filtered and solvent was removed under vacuum. The residue was purified by silica gel column to provide 1 g of 287-1 (yield: 71.5%) as a yellow solid. Mass (m/z): 241.2 [M+H]⁺.

Step 2. Preparation of ethyl (E)-3-(4-oxocyclohexyl)acrylate (287-2)

287-1 (1 g, 4.1 mmol) and HCl in THF (20 mL, 2 N) were placed in a flask and stirred at 250C for 18 hrs. Excess THF was removed under vacuum and the residue was purified by silica gel column to provide 500 mg of 287-2 (yield: 61.9%) as a yellow oil. Mass (m/z): 197.2 [M+H]⁺.

Step 3. Preparation of ethyl (E)-3-(4-((4-(tert-butyl)phenyl)amino)cyclohexyl)acrylate (287-3)

To a solution of 287-2 (200 mg, 1 mmol), 4-(tert-butyl)aniline (150 mg, 1 mmol) and NaBH₃CN (120 mg, 2 mmol) in MeOH (10 mL). The reaction was stirred at R.T. for 18 hours.

The solid was filtered and solvent was removed under vacuum. The residue was purified by silica gel column to provide 220 mg of 287-3 (yield: 66.7%) as a yellow solid. Mass (m/z): 330.3 [M+H]⁺.

Step 4. Preparation of ethyl 3-(4-((4-(tert-butyl)phenyl)amino)cyclohexyl) propanoate (287-4)

A solution of 287-3 (220 mg, 0.67 mmol), Pd/C (20 mg) in MeOH (10 mL) was stirred at 25° C. under H₂ atmosphere for 18 hours. The solid was filtered and solvent was removed under vacuum. The residue was purified by silica gel column to provide 150 mg of 287-4 (yield: 67.4%) as a yellow solid. Mass (m/z): 332.3 [M+H]⁺.

Step 5. Preparation of 3-(4-((4-(tert-butyl)phenyl)amino)cyclohexyl) propanoic acid (287-5)

To a solution of 287-4 (150 mg, 0.45 mmol) in H₂O (5 mL) and THF (5 mL) was added NaOH (90 mg, 2.25 mmol). The mixture was stirred at 25° C. overnight. Ethyl acetate was added to the reaction mixture and the mixture was filtered through Celite. The filtrate was washed with water and saturated brine. The organic layer was dried over magnesium sulfate and concentrated under reduced pressure. The residue was purified by silica gel column to provide 100 mg of 287-5 (yield: 73.1%) as yellow oil. Mass (m/z): 304.2 [M+H]⁺.

Step 6. Preparation of 3-(4-((4-(tert-butyl)phenyl)amino)cyclohexyl) propenamide (287-6)

A solution of 287-5 (150 mg, 0.5 mmol), NH₄Cl (80 mg, 1.5 mmol), HATU (281 mg, 0.75 mmol) and DIEA (191 mg, 1.5 mmol) in DMF (10 mL) was stirred at R.T. for 18 hours. The solid was filtered and solvent was removed under vacuum. The residue was purified by silica gel column to provide 200 mg of 287-6 (yield: 90.1%) as a yellow solid. Mass (m/z): 302.9 [M+H]⁺.

Step 7. Preparation of N-(4-(3-aminopropyl)cyclohexyl)-4-(tert-butyl)aniline (287)

A solution of 287-6 (60 mg, 0.2 mmol), BH₃-THF (10 mL) in THF (5 mL) was stirred at 70° C. for 18 hours. The solid was filtered and solvent was removed under vacuum. The residue was purified by prep-HPLC (column-Xbridge-C18 150×19 mm, 5 μm; Mobile phase: ACN-H₂O (0.1% FA), 25%-40%) to afford 287 (7.0 mg) as a white solid. ¹H NMR (400 MHZ, CD₃OD) δ 7.14 (d, J-8.6 Hz, 2H), 6.62 (d, J-8.6 Hz, 2H), 3.49-3.43 (m, 1H), 2.64 (t, J-7.4 Hz, 2H), 1.79-1.61 (m, 4H), 1.58-1.47 (m, 4H), 1.40 (s, 3H), 1.31 (m, 2H), 1.25 (s, 9H). Mass (m/z): 289.0 [M+H]⁺.

### Compound 288

### N-(((1s, 4s)-4-aminocyclohexyl)methyl)-4-(tert-butyl)aniline

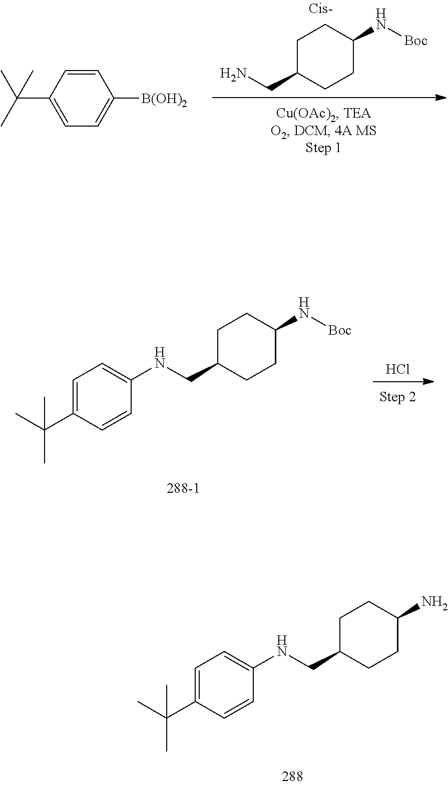 [SMILES: CC(C)(C)c1ccc(B(O)O)cc1 ; CC(C)(C)OC(=O)N[C@H]1CC[C@@H](CN)CC1 ; CC(C)(C)OC(=O)N[C@H]1CC[C@@H](CNc2ccc(C(C)(C)C)cc2)CC1 ; Cl ; CC(C)(C)c1ccc(NC[C@H]2CC[C@@H](N)CC2)cc1]

Step 1. Preparation of tert-butyl ((1s,4s)-4-(((4-(tert-butyl)phenyl)amino)methyl)cyclohexyl)carbamate (288-1)

A 100-mL round-bottom flask was charged with tert-butyl ((1s,4s)-4-(aminomethyl)cyclohexyl)carbamate (155 mg, 0.67 mmol, 1.1 eq), (4-(tert-butyl)phenyl) boronic acid (100 mg, 0.56 mmol, 1.00 eq), Cu (OAc) 2 (204 mg, 1.1 mmol, and 2.00 eq) and TEA (284 mg, 2.8 mmol, 5.00 eq) and 4 Å MS (1 g). The reaction was stirred at R.T. under O₂ atmosphere for 18 hours. The solids were filtered and solvent was removed under vacuum. The residue was purified by Flash Chromatography to give 288-1 (0.1 g, 49.2% yield) as a yellow solid. MS (m/z) 361.3 [M+H]⁺.

Step 2. Preparation of N-(((1s,4s)-4-aminocyclohexyl)methyl)-4-(tert-butyl)aniline (288)

288-1 (100 mg, 0.28 mmol) and HCl in 1,4-dioxane (10 mL, 4N) were placed in a flask stirred at 25° C. for 16 hrs. Excess 1,4-dioxane was distilled under vacuum and the residue was purified by Flash Chromatography to afford 288 (59.1 mg 82.0%) as a white solid. ¹H NMR (400 MHZ, CD₃OD) δ 7.55-7.52 (m, 2H), 7.30-7.26 (m, 2H), 3.35-3.30 (m, 3H), 1.97 (dd, J=7.1, 3.6 Hz, 1H), 1.85-1.71 (m, 6H), 1.65-1.57 (m, 2H), 1.31 (s, 9H). MS (m/z) 261.2 [M+H]⁺.

### Compound 289

### 1-N-(4-(sec-butyl)phenyl)cyclohexane-1,4-diamine

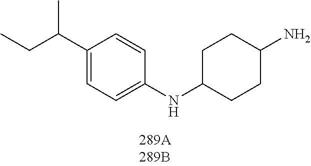 [SMILES: CCC(C)c1ccc(NC2CCC(N)CC2)cc1]

The title compounds 289A (64.1 mg) as a white solid and 289B (51.4 mg) as a white solid were prepared from tert-butyl (4-((4-(sec-butyl)phenyl)amino)cyclohexyl)carbamate (100 mg, 0.28 mmol), 1,4-dioxane (5 mL) and HCl/1,4-dioxane (10 mL) according to the procedure for 37. 289A: ¹HNMR (400 MHZ, CD₃OD) δ 6.93 (d, J=8.4 Hz, 2H), 6.61 (d, J=8.6 Hz, 2H), 3.22-3.11 (m, 1H), 2.70-2.60 (m, 1H), 2.43 (dd, J=14.2, 7.0 Hz, 1H), 2.06 (d, J=11.8 Hz, 2H), 1.91 (d, J=11.8 Hz, 2H), 1.59-1.47 (m, 2H), 1.31-1.21 (m, 3H), 1.16 (d, J=7.0 Hz, 4H), 0.78 (t, J=7.4 Hz, 3H). Mass (m/z): 247.2 [M+H]⁺. HPLC: Rt=3.770 min (Column: XBRIDGE 3.5 μm, 2.1*50 mm, Mobile phase: H₂O (0.05% TFA)-ACN (0.05% TFA), ACN from 0 to 60% over 7 minutes, 7-8 min, ACN from 60% to 100%, Flow rate: 0.8 mL/min). 289B: ¹HNMR (400 MHZ, CD₃OD) δ 6.93 (d, J=8.4 Hz, 2H), 6.63 (d, J=8.6 Hz, 2H), 3.44 (s, 1H), 2.83 (d, J=8.2 Hz, 1H), 2.46-2.41 (m, 1H), 1.77-1.49 (m, 10H), 1.17 (d, J=6.8 Hz, 3H), 0.79 (t, J=7.4 Hz, 3H). Mass (m/z): 247.2 [M+H]⁺. HPLC: Rt=3.962 min (Column: XBRIDGE 3.5 μm, 2.1*50 mm, Mobile phase: H₂O (0.05% TFA)-ACN (0.05% TFA), ACN from 0 to 60% over 7 minutes, 7-8 min, ACN from 60% to 100%, Flow rate: 0.8 mL/min).

### Compound 290

### N-(4-(1-aminoethyl)cyclohexyl)-4-(tert-butyl)aniline

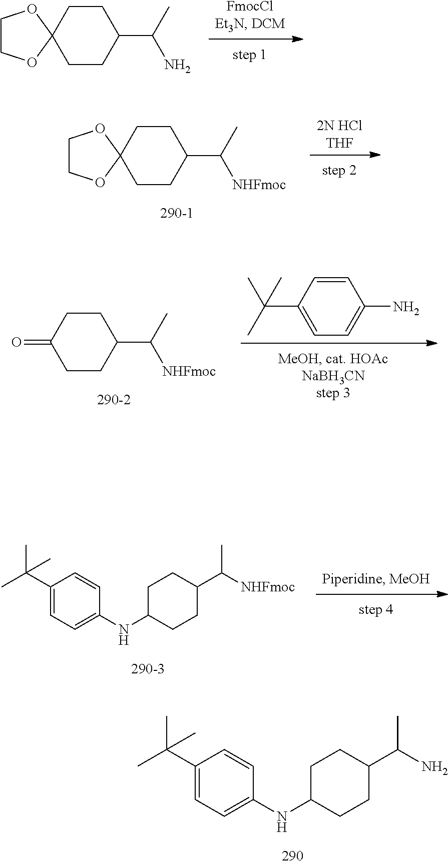

Step 1. Preparation of (9H-fluoren-9-yl)methyl (1-(1,4-dioxaspiro[4.5]decan-8-yl)ethyl)carbamate (290-1) To a solution of 1-(1,4-dioxaspiro[4.5]decan-8-yl) ethan-1-amine (0.5 g, 2.7 mmol) in DCM (10 mL) was added DIEA (697 mg, 5.4 mmol) and FmocCl (600 mg, 2.7 mmol). Then the mixture was stirred at 25° C. for 2 hrs. LCMS showed the reaction was completed. The mixture was added into H₂O, extracted with EA (20 mL*3). The combined organic layers were washed with brine (40 mL), dried over sodium sulfate, filtered and concentrated. The residue was purified by combi-flash with EA/PE (1:1) to give 290-1 (0.5 g, 45.4% yield) as a yellow oil. MS (m/z) 407.8 [M+H]⁺.

Step 2. Preparation of (9H-fluoren-9-yl)methyl (1-(4-oxocyclohexyl)ethyl)carbamate (290-2)

To a solution of 290-1 (0.25 g, 0.61 mmol) in THF (4 mL) was added 2N HCl (4 mL) at 25° C. Then the mixture was stirred at 25° C. for 10 h. LCMS showed the reaction was completed. The reaction was concentrated. The residue was purified by combi-flash with EA/PE (1:2) to afford 290-2 (0.17 g, 76.2% yield) as yellow oil. MS (m/z) 364.2 [M+H]⁺.

Step 3. Preparation of (9H-fluoren-9-yl)methyl (1-(4-((4-(tert-butyl)phenyl)am ino) cyclohexyl)ethyl)carbamate (290-3)

To a solution of 290-2 (100 mg, 0.27 mmol) in MeOH (5 mL) and a drop of AcOH was added 4-(tert-butyl)aniline (41 mg, 0.27 mmol) and the mixture was stirred at 50° C. for 1 h. Then the mixture was added NaBH₃CN (51 mg, 0.81 mmol) after cooling to 25° C. Then the mixture was stirred at 25° C. for 2 hrs. LCMS showed the reaction was completed. The reaction was quenched with water (10 mL), extracted with EA (10 mL*3). The combined organic layers were washed with brine (20 mL), dried over sodium sulfate, filtered and concentrated. The residue was purified by combi-flash with EA/PE (1:2) to give 290-3 (0.1 g, 73.5% yield) as a yellow solid. MS (m/z) 496.8 [M+H]⁺.

Step 4. Preparation of N-(4-(1-aminoethyl)cyclohexyl)-4-(tert-butyl)aniline (290)

To a solution of 290-3 (0.1 g, 0.20 mmol) in MeOH (2 mL) was added piperidine (2 mL) at 25° C. Then the mixture was stirred at 25° C. for 4 h. LCMS showed the reaction was completed.

The reaction was concentrated. The residue was purified by prep-HPLC to afford 290 (10 mg, 13% yield) as a yellow solid. ¹H NMR (300 MHz, CD₃OD) δ 7.63 (d, J=7.2 Hz, 2H), 7.46-7.36 (m, 2H), 3.53-3.38 (m, 1H), 3.20-3.10 (m, 1H), 2.10 (d, J=11.2 Hz, 1H), 2.00-1.42 (m, 8H), 1.35 (s, 9H), 1.27-1.22 (m, 3H). MS (m/z) 275.3 [M+H]⁺.

### Compound 291

### N-(4-(aminomethyl)cyclohexyl)-4-propylaniline

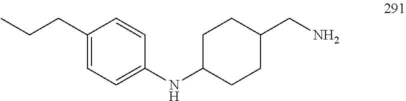 [SMILES: CCCc1ccc(NC2CCC(CN)CC2)cc1]

The title compound 291 (30.6 mg, 28.8%) as a white solid was prepared from tert-butyl ((4-((4-propylphenyl)amino)cyclohexyl)methyl)carbamate (150 mg, 0.43 mmol), 1,4-dioxane (10 mL) and 1,4-dioxane/HCl (10 mL) according to the procedure for 224. ¹H NMR (400 MHZ, DMSO-d₆) δ 6.86 (d, J=7.6 Hz, 2H), 6.46 (d, J=7.7 Hz, 2H), 5.13 (br, 1H), 3.07 (br, 1H), 2.63 (br, 2H), 2.36 (t, J=7.2 Hz, 2H), 1.97 (br, 2H), 1.81 (br, 2H), 1.50-1.45 (m, 3H), 1.10-0.95 (m, 4H), 0.85 (t, J=7.0 Hz, 3H). Mass (m/z): 247.3 [M+H]⁺.

### Compound 292

### N-(4-(2-aminoethyl)cyclohexyl)-4-propylaniline

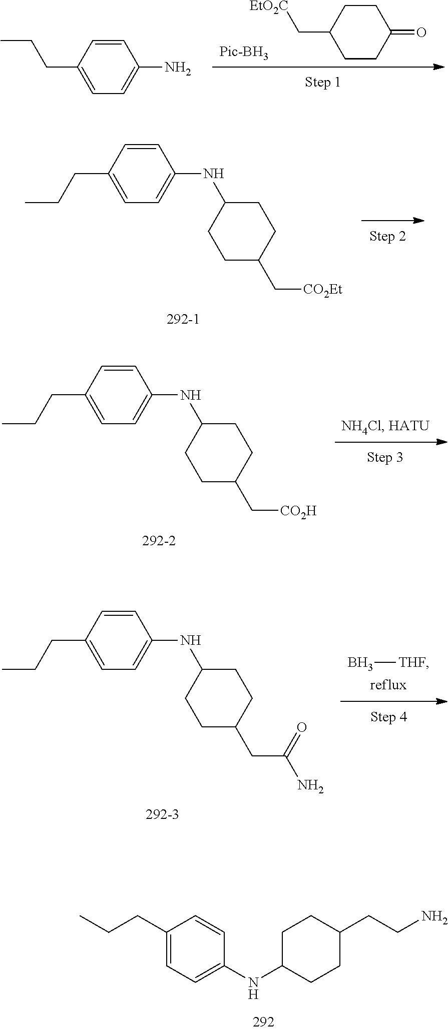

Step 1. Preparation of ethyl 2-(4-((4-propylphenyl)amino)cyclohexyl) acetate (292-1)

A solution of 4-propylaniline (500 mg, 3.7 mmol), ethyl 2-(4-oxocyclohexyl) acetate (681 mg, 3.7 mmol) and Pic-BH₃ (474 mg, 4.44 mmol) in H₂O (9 mL) and HOAc (1 mL) was stirred at R.T. for 18 hours. The solid was filtered and solvent was removed under vacuum. The residue was purified by silica gel column to provide 600 mg of 292-1 (yield: 53.3%) as a yellow solid. Mass (m/z): 304.3 [M+H]⁺.

Step 2. Preparation of 2-(4-((4-propylphenyl)amino)cyclohexyl) acetic acid (292-2)

To a solution of 292-1 (600 mg, 2 mmol) in H₂O (5 mL) and THF (5 mL) was added LiOH (780 mg, 20 mmol). The mixture was stirred at 25° C. overnight. Ethyl acetate was added to the reaction mixture and the mixture was filtered through Celite. The filtrate was washed with water and saturated brine. The organic layer was dried over magnesium sulfate and concentrated under reduced pressure. The residue was purified by silica gel column to provide 200 mg of 292-2 (yield: 36.2%) as a yellow oil. Mass (m/z): 276.2 [M+H]⁺.

Step 3. Preparation of 2-(4-((4-propylphenyl)amino)cyclohexyl)acetamide (292-3)

A solution of 292-2 (300 mg, 1.1 mmol), NH₄Cl (70 mg, 1.3 mmol), HATU (621 mg, 1.6 mmol) and DIEA (281 mg, 2.2 mmol) in DMF (10 mL) was stirred at R.T. for 18 hours. The solid was filtered and solvent was removed under vacuum. The residue was purified by silica gel column to provide 200 mg of 292-3 (yield: 66.1%) as a yellow solid. Mass (m/z): 275.2 [M+H]⁺.

Step 4. Preparation of N-(4-(2-aminoethyl)cyclohexyl)-4-propylaniline (292)

A solution of 292-3 (200 mg, 0.73 mmol), BH₃-THF (10 mL) in THF (5 mL) was stirred at 70° C. for 18 hours. The solid was filtered and solvent was removed under vacuum. The residue was purified by prep-HPLC (column-Xbridge-C18 150×19 mm, 5 μm; Mobile phase: ACN-H₂O (0.1% FA), 25%-40%) to afford 292 (13.4 mg) as a white solid. Mass (m/z): 261.3 [M+H]⁺. ¹H NMR (400 MHZ, DMSO-d₆) δ 6.81 (d, J=8.4 Hz, 2H), 6.42 (d, J=8.4 Hz, 2H), 3.09-2.97 (m, 1H), 2.80-2.68 (m, 2H), 2.36-2.28 (m, 2H), 1.92 (d, J=11.6 Hz, 2H), 1.68 (d, J=12.0 Hz, 2H), 1.51-1.36 (m, 4H), 1.34-1.20 (m, 1H), 1.11-0.89 (m, 4H), 0.81 (t, J=7.4 Hz, 3H).

### Compound 293

### (1r,4r)-N1-(4-(3-ethoxypropyl)phenyl)cyclohexane-1,4-diamine

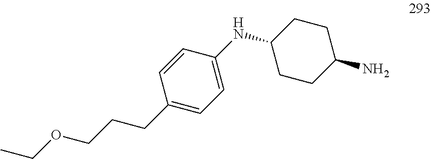 [SMILES: CCOCCCc1ccc(N[C@H]2CC[C@H](N)CC2)cc1]

The mixture of tert-butyl ((1r,4r)-4-((4-(3-ethoxypropyl)phenyl)amino)cyclohexyl)carbamate (300 mg, 0.79 mmol) in 1,4-dioxane (10 mL) and 1,4-dioxane/HCl (10 mL) was stirred for 16 hour at 25° C. After reaction was completed, solvent was removed under vacuum, and the residue was purified by prep-HPLC (column-Xbridge-C18 150×21.2 mm, 5 μm; Mobile phase: ACN-H₂O (0.1% FA), 2%-10%) to afford the desired product as a white solid. (30.5 mg, 13.7%). ¹H NMR (400 MHZ, DMSO-d₆) δ 6.87 (d, J=8.2 Hz, 2H), 6.48 (d, J=8.3 Hz, 2H), 3.41-3.35 (m, 2H), 3.31 (t, J=6.5 Hz, 2H), 3.08 (br, 1H), 2.90 (br, 1H), 2.45-2.41 (m, 2H), 2.02-1.91 (m, 4H), 1.73-1.64 (m, 2H), 1.42-1.36 (m, 2H), 1.24-1.12 (m, 2H), 1.09 (d, J=7.0 Hz, 3H). Mass (m/z): 277.3 [M+H]⁺.

### Compound 294

### 2-((4-((4-(tert-butyl)phenyl)amino)cyclohexyl)amino)ethan-1-ol

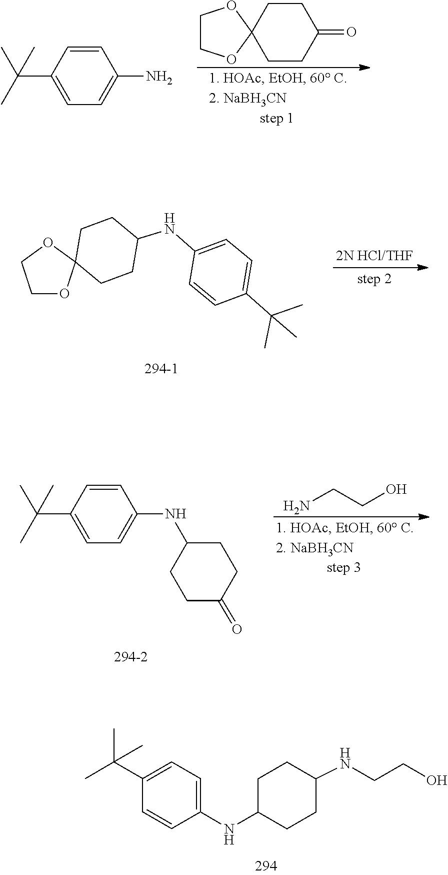

Step 1. Preparation of N-(4-(tert-butyl)phenyl)-1,4-dioxaspiro[4.5]decan-8-amine (294-1) To a solution of 4-(tert-butyl)aniline (1 g, 6.71 mmol) in MeOH (10 mL) was added 1,4-dioxaspiro[4.5]decan-8-one (1.04 g, 6.71 mmol) and the mixture was stirred at 60° C. for 1 h. Then sodium cyanoborohydride (1.27 g, 20.13 mmol) was added to the mixture. The reaction was stirred for 3 h at R.T. Quenched with water (50 mL), the reaction was extracted by EA (10 mL) for 3 times. The organic phase was dried over sodium sulfate. removed under vacuum and the residue was purified by flash chromatography to afford the desired product (1.6 g, 82.6%) as a white solid. Mass (m/z): 289.9 [M+H]⁺.

Step 2. Preparation of 4-((4-(tert-butyl)phenyl)amino)cyclohexan-1-one (294-2)

To a solution of N-(4-(tert-butyl)phenyl)-1,4-dioxaspiro[4.5]decan-8-amine (1.6 g, 5.54 mmol) in THF (5 mL) was added HCl in THF (2 N, 5 mL) and the mixture was stirred for 2 h. Quenched with NaHCO₃ (10 mL), the reaction was extracted by EA (10 mL) for 3 times. The organic phase was dried over sodium sulfate, removed under vacuum and the product (0.9 g, 66.2%) as a white solid. Mass (m/z): 246.2 [M+H]⁺.

Step 3. Preparation of 2-((4-((4-(tert-butyl)phenyl)amino)cyclohexyl)amino)ethan-1-ol (294)

To a solution of 4-((4-(tert-butyl)phenyl)amino)cyclohexan-1-one (100 mg, 0.41 mmol) in MeOH (5 mL) was added 2-aminoethan-1-ol (25 mg, 0.41 mmol) and acetic acid (2.46 mg, 0.041 mmol). Then the mixture was stirred at 60° C. for 1 h. After reaction was cooled to R.T., Sodium cyanoborohydride (77.49 mg, 12.3 mmol) was added. The reaction was stirred for 3 h at R.T. Quenched with water (10 mL), the reaction was extracted by EA (10 mL) for 3 times. The organic phase was dried over sodium sulfate, removed under vacuum and the residue was purified by prep-HPLC (column-Xbridge-C_{18 150×21.2} mm, 5 μm; Mobile phase: ACN-H₂O (0.1% FA), 40%-60%) to afford the desired product (mixture; 113 mg, 95%) as yellow solid. ¹H NMR (400 MHZ, DMSO-d₆) δ 7.06 (d, J=8.8 Hz, 2H), 6.49 (dd, J=13.6 Hz, 8.4 Hz, 2H), 5.13 (s, 1H), 3.49 (s, 3H), 3.20 (m, 1H), 2.67 (m, 2H), 2.51 (s, 1H), 1.95 (t, J=14.0 Hz, 2H), 1.69-1.49 (m, 3H), 1.20 (s, 12H). Mass (m/z): 290.9 [M+H]⁺. HPLC: Rt: 3.505 min, 3.849 min (Column: XBRIDGE 2.1*50 mm, 3.5 μm; Mobile Phase: H₂O (0.05% TFA)/ACN (0.05% TFA), ACN from 0% to 60% over 7 minutes, 7-8 min, ACN from 60% to 100%; 0.8 mL/min).

### Compound 295

### 2-((4-((4-(tert-pentyl)phenyl)amino)cyclohexyl)amino)ethan-1-ol

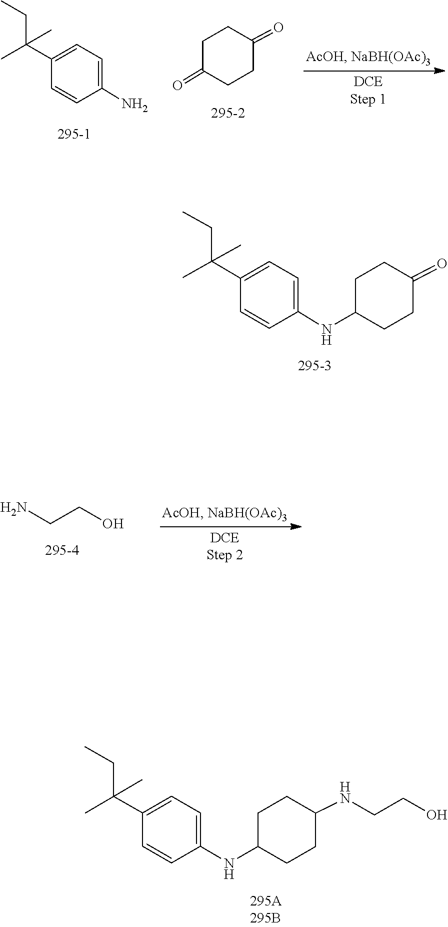

Step 1. Preparation of 4-((4-(tert-pentyl)phenyl)amino)cyclohexan-1-one (295-3)

The compound 295-3 (99 mg) was prepared in a total yield of 77% as a white solid from 4-(tert-pentyl)aniline (26 mg, 0.1 mmol) and cyclohexane-1,4-dione (66 mg, 0.6 mmol) according to the procedure for compound 4. Mass (m/z): 260.2 [M+H]⁺.

Step 2. Preparation of 2-((4-((4-(tert-pentyl)phenyl)amino)cyclohexyl)amino)ethan-1-ol (295)

A mixture of 4-((4-(tert-pentyl)phenyl)amino)cyclohexan-1-one (26 mg, 0.1 mmol), 2-aminoethan-1-ol (4.4 mg, 0.12 mmol) and acetic acid (0.06 ml, 0.1 mmol) in DCE (5 mL) was stirred for 1 h at room temperature. Afterwards sodium triacetoxyborohydride (42 mg, 0.2 mmol) was added and the mixture was stirred overnight. To the mixture, saturated NaHCO₃aq. was added and the mixture was extracted with ethyl acetate. The combined organic layers were dried over MgSO₄ and evaporated. The mixture was purified by preparative HPLC (Column: X Select-CSH-Prep 5 μm OBD, 19*150 mm; ACN/water (0.5% TFA)=5%-40%-95%-95%-5%, 0 min-10 min-10.5 min-11.5 min-13 min) to afford compound 295A (Rt=6.58 min) as a white solid and compound 295B (Rt=7.83 min) as a white solid. 295A (7.1 mg, 24%): ¹H NMR (400 MHZ, DMSO-d₆) δ 8.79 (s, 1H), 7.03 (d, J=8.4 Hz, 2H), 6.55 (d, J=8.4 Hz, 2H), 5.29 (s, 1H), 5.18 (s, 1H), 3.68 (m, 2H), 3.45 (m, 1H), 3.09 (m, 1H), 2.99 (m, 2H), 1.90-1.75 (m, 6H), 1.63-1.48 (m, 4H), 1.16 (s, 6H), 0.60 (t, J=7.2 Hz, 3H). Mass (m/z): 305.1 [M+H]⁺. 295B (10.2 mg, 34%): ¹H NMR (400 MHZ, DMSO-d₆) δ 8.96 (s, 1H), 7.03 (d, J=8.4 Hz, 2H), 6.61 (d, J=8.4 Hz, 2H), 5.28 (s, 1H), 5.18 (s, 1H), 3.68 (m, 2H), 3.44 (m, 1H), 3.09 (m, 1H), 2.97 (m, 2H), 2.17-1.73 (m, 4H), 1.58-1.41 (m, 4H), 1.22 (m, 2H), 1.15 (s, 6H), 0.59 (t, J=7.2 Hz, 3H). Mass (m/z): 305.1 [M+H]⁺.

### Compound 296

### (1-amino-4-((4-(tert-pentyl)phenyl)amino)cyclohexyl)methanol

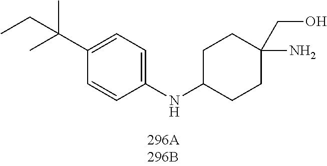 [SMILES: CCC(C)(C)c1ccc(NC2CCC(N)(CO)CC2)cc1]

The title compound 296 was prepared from 1-amino-4-((4-(tert-pentyl)phenyl)amino)cyclohexane-1-carboxylic acid (31 mg, 0.1 mmol) according to the procedure for compound 4. The mixture was purified by preparative HPLC (ACN/water (5% oTFA=May 30, 1995-95-5, 0-10-10.5-11.5-13.0 min) to afford compound 296A (Rt=4.33 min) as a rosy brown solid and compound 296B (Rt=3.95 min) as a rosy brown solid. 296A (15.6 mg, 54%): ¹H NMR (400 MHZ, DMSO-d₆) δ 7.93 (s, 2H), 7.02 (d, J=8.4 Hz, 2H), 6.51 (d, J=8.4 Hz, 2H), 5.47 (s, 1H), 5.07 (s, 1H), 3.46 (s, 2H), 3.23 (m, 1H), 1.95-1.68 (m, 4H), 1.65-1.40 (m, 6H), 1.16 (s, 6H), 0.61 (t, J=7.2 Hz, 3H). Mass (m/z): 291.2 [M+H]⁺. 296B (10.2 mg, 35%): ¹H NMR (400 MHZ, DMSO-d₆) δ 7.94 (s, 2H), 7.01 (d, J=8.4 Hz, 2H), 6.51 (d, J=8.4 Hz, 2H), 5.48 (s, 1H), 5.27 (s, 1H), 3.46 (s, 2H), 3.15 (m, 1H), 1.95-1.80 (m, 4H), 1.65-1.48 (m, 4H), 1.35-1.23 (m, 3H), 1.15 (s, 6H), 0.60 (t, J=7.2 Hz, 3H). Mass (m/z): 291.2 [M+H]⁺.

### Compound 297

### 4-(4,4-dimethylcyclohexyl)-N-(1-(pyrrolidin-1-yl) propan-2-yl)aniline

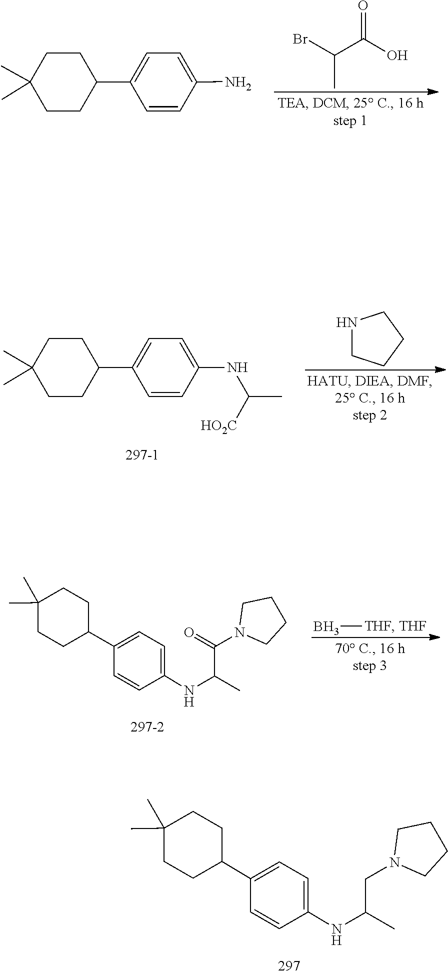

Step 1. (4-(4,4-dimethylcyclohexyl)phenyl) alanine (297-1)

A mixture of 4-(4,4-dimethylcyclohexyl)aniline (100 mg, 0.49 mmol), 2-bromopro-panoic acid (112.85 mg, 0.74 mmol), TEA (99.53 mg, 0.98 mmol) in DCM (5 mL) was stirred overnight at 25° C. It was quenched by H₂O (30 mL) and extracted with DCM (3×30 mL). The organic layers were combined, washed with brine NaCl (2×30 mL) and dried with MgSO₄, filtered and concentrated to afford target product (100 mg, 59%) as yellow oil. Mass (m/z): 276.0 [M+H]⁺.

Step 2. 2-((4-(4,4-dimethylcyclohexyl)phenyl)amino)-1-(pyrrolidin-1-yl) propan-1-one (297-2)

To a solution of 297-1 (100 mg, 0.36 mmol), pyrrolidine (51.65 mg, 0.72 mmol) in DMF (7 ml) was added HATU (207.1 mg, 0.54 mmol) and DIEA (93.86 mg, 0.72 mmol). Then the mixture was stirred overnight at 25° C. It was quenched by H₂O (30 mL) and extracted with EA (3×30 mL). The organic layers were combined, washed with brine NaCl (2×30 mL) and dried with MgSO₄, filtered and concentrated. The crude product was applied onto a silica gel column (4 g) eluted with PE: EA (10:1) to give product (50 mg, 13.4%). Mass (m/z): 329.3 [M+H]⁺.

Step 3. Preparation of 4-(4,4-dimethylcyclohexyl)-N-(1-(pyrrolidin-1-yl) propan-2-yl)aniline (297)

To a solution of 297-2 (50 mg, 0.15 mmol), 2-(4-methylpiperazin-1-yl) acetic acid (66 mg, 0.42 mmol) in THF (5 mL) was added BH₃-THF (10 mL). Then the mixture was stirred overnight at rt. The reaction was concentrated under vacuum. The residue was purified by prep-TLC to afford the desired product 297 (19.8 mg) as a white solid. ¹H NMR (400 MHZ, CD₃OD) δ 7.06-6.96 (m, 2H), 6.72-6.59 (m, 2H), 3.90-3.79 (m, 1H), 3.27-3.17 (m, 3H), 3.10 (d, J=6.6 Hz, 2H), 2.34-2.21 (m, 1H), 2.01 (d, J=6.6 Hz, 4H), 1.67-1.30 (m, 9H), 1.15 (d, J=6.4 Hz, 3H), 0.96 (s, 3H), 0.92 (s, 3H). Mass (m/z): 315.2 [M+H]⁺.

### Compound 298

### 4-((4-(tert-pentyl)phenyl)amino)cyclohexan-1-ol

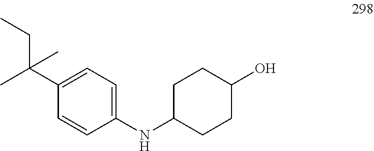 [SMILES: CCC(C)(C)c1ccc(NC2CCC(O)CC2)cc1]

The title compound 298 (47.8 mg) was prepared in a total yield of 61.0% as a light-yellow solid from 4-(tert-pentyl)aniline (49.2 mg, 0.3 mmol), 4-hydroxycyclohexan-1-one (68.4 mg, 0.6 mmol) and Na(AcO)₃BH (127 mg, 0.6 mmol) according to the procedure for 4. ¹H NMR (400 MHZ, DMSO-d₆) δ 7.06-6.80 (m, 2H), 6.59-6.27 (m, 2H), 5.12 (d, J=8.0 Hz, 0.5H), 5.04 (d, J=8.0 Hz, 0.5H), 4.50 (d, J=4.4 Hz, 0.5H), 4.34 (d, J=3.0 Hz, 0.5H), 3.71-3.59 (m, 0.5H), 3.36 (s, 0.5H), 3.21-3.10 (m, 0.5H), 3.02 (s, 0.5H), 1.87 (d, J=12.8 Hz, 0.5H), 1.78 (d, J=12.4 Hz, 0.5H), 1.62-1.41 (m, 10H), 1.12 (s, 6H), 0.57 (td, J=7.4, 2.0 Hz, 3H). Mass (m/z): 262.3 [M+H]⁺.

### Compound 299

### N1-(4-(tert-pentyl)phenyl)cyclohexane-1,4-diamine

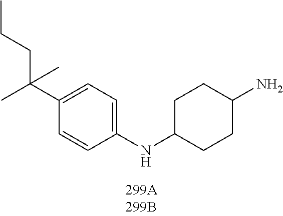 [SMILES: CCCC(C)(C)c1ccc(NC2CCC(N)CC2)cc1]

The title compound 299A and 299B were prepared according to the procedure for compound 232. The crude residue was purified by preparative TLC (H₂O/MeOH/DCM=0.1/1/5) to afford compound 299A in 30.9% yield as a white solid and 299B in 25.3% yield as a white solid. 299A: ¹H NMR (400 MHZ, DMSO-d₆) δ 7.61 (s, 2H), 7.05-6.98 (m, 2H), 6.56-6.48 (m, 2H), 5.20 (d, J=5.6 Hz, 1H), 3.43-3.36 (m, 1H), 3.07 (p, J=6.4 Hz, 1H), 1.85-1.56 (m, 6H), 1.51-1.36 (m, 2H), 1.17 (s, 6H), 1.07-0.95 (m, 2H), 0.77 (t, J=7.3 Hz, 3H). Mass (m/z): 275.3 [M+H]⁺. HPLC: Rt=1.816 mins (Agilent, poroshell 120, SB—C18 2.7 μm, 4.6×50 mm, ACN/Water (0.1% FA)=5%-5%-95%-95%-95%-5%, 0-0.5 min-10 min-10.5 min-12.0 min). 299B: ¹H NMR (400 MHZ, DMSO-d₆) δ 7.04-6.96 (m, 2H), 6.67-6.45 (m, 4H), 5.15 (d, J=8.0 Hz, 1H), 3.13-3.01 (m, 1H), 2.90-2.79 (m, 1H), 2.07-1.84 (m, 4H), 1.51-1.41 (m, 2H), 1.38-1.28 (m, 2H), 1.21-1.11 (m, 7H), 1.02-0.96 (m, 1H), 0.77 (t, J=7.2 Hz, 3H). Mass (m/z): 275.3 [M+H]⁺. HPLC: Rt=1.561 mins (Agilent, poroshell 120, SB—C18 2.7 μm, 4.6×50 mm, ACN/Water (0.1% FA)=5%-5%-95%-95%-95%-5%, 0-0.5 min-10 min-10.5 min-12.0 min).

### Compound 300

### N1-(4-(tert-butyl)-3-(3-methoxypropoxy) phenyl)cyclohexane-1,4-diamine

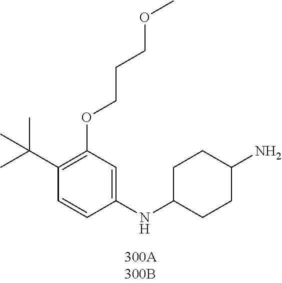 [SMILES: COCCCOc1cc(NC2CCC(N)CC2)ccc1C(C)(C)C]

The title compound 300A and 300B were prepared according to the procedure for compound 254. The crude residue was purified by preparative TLC (H₂O/MeOH/DCM=0.1/1/5) to afford compound 300A in 33.4% yield as a white solid and 300B in 25.3% yield as a white solid. 300A: ¹H NMR (400 MHZ, Methanol-d₄) δ 6.98 (d, J=8.4 Hz, 1H), 6.30 (d, J=2.3 Hz, 1H), 6.16 (dd, J=8.4, 2.3 Hz, 1H), 3.99 (t, J=6.2 Hz, 2H), 3.60 (t, J=6.2 Hz, 2H), 3.55-3.50 (m, 1H), 3.33 (s, 3H), 3.23-3.17 (m, 1H), 2.04 (q, J=6.2 Hz, 2H), 1.88-1.73 (m, 8H), 1.30-1.29 (m, 9H). Mass (m/z): 335.3 [M+H]⁺. HPLC: Rt=4.800 mins (Agilent, poroshell 120, SB—C18 2.7 μm, 4.6×50 mm, ACN/Water (0.1% FA)=5%-5%-95%-95%-95%-5%, 0-0.5 min-10 min-10.5 min-12.0 min).

300B: ¹H NMR (400 MHZ, Methanol-d₄) δ 6.99 (d, J=8.4 Hz, 1H), 6.30 (d, J=2.3 Hz, 1H), 6.18 (dd, J=8.4, 2.3 Hz, 1H), 3.99 (t, J=6.2 Hz, 2H), 3.60 (t, J=6.2 Hz, 2H), 3.33 (s, 3H), 3.28-3.17 (m, 4H), 3.16-3.02 (m, 1H), 2.18-2.12 (m, 2H), 2.10-2.02 (m, 4H), 1.49 (td, J=12.5, 3.3 Hz, 2H), 1.30 (s, 9H), 1.28-1.21 (m, 2H). Mass (m/z): 335.3 [M+H]⁺. HPLC: Rt=4.284 mins (Agilent, poroshell 120, SB—C18 2.7 μm, 4.6×50 mm, ACN/Water (0.1% FA)=5%-5%-95%-95%-95%-5%, 0-0.5 min-10 min-10.5 min-12.0 min).

### Compound 301

### N1-(4-(tert-butyl)-3-(2-methoxyethoxy) phenyl)cyclohexane-1,4-diamine

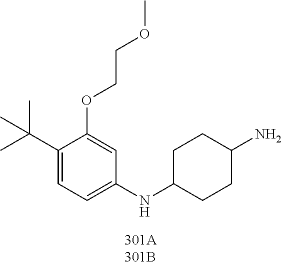 [SMILES: COCCOc1cc(NC2CCC(N)CC2)ccc1C(C)(C)C]

The title compound 301A and 301B were prepared according to the procedure for compound 254. The crude residue was purified by preparative TLC (H₂O/MeOH/DCM=0.1/1/5) to afford compound 301A in 31.2% yield as a white solid and 301B in 21.3% yield as a white solid. 301A: ¹H NMR (400 MHZ, Methanol-d₄) δ 8.07 (d, J=8.4 Hz, 1H), 7.38 (s, 1H), 7.25 (d, J=8.4 Hz, 1H), 5.15-5.08 (m, 2H), 4.88-4.81 (m, 2H), 4.70-4.54 (m, 1H), 4.49 (s, 3H), 4.34-4.25 (m, 1H), 2.97-2.77 (m, 8H), 2.38 (s, 9H). Mass (m/z): 321.3 [M+H]⁺. HPLC: Rt=4.678 mins (Agilent, poroshell 120, SB—C18 2.7 μm, 4.6×50 mm, ACN/Water (0.1% FA)=5%-5%-95%-95%-95%-5%, 0-0.5 min-10 min-10.5 min-12.0 min). 301B: ¹H NMR (400 MHZ, Methanol-d₄) δ 6.98 (d, J=8.4 Hz, 1H), 6.27 (d, J=2.3 Hz, 1H), 6.17 (dd, J=8.4, 2.3 Hz, 1H), 4.06-3.99 (m, 2H), 3.79-3.71 (m, 2H), 3.40 (s, 3H), 3.23-3.10 (m, 1H), 3.10-2.98 (m, 1H), 2.19-2.01 (m, 4H), 1.56-1.44 (m, 2H), 1.30 (s, 9H), 1.28-1.19 (m, 2H). Mass (m/z): 321.3 [M+H]⁺. HPLC: Rt=4.231 mins (Agilent, poroshell 120, SB—C18 2.7 μm, 4.6×50 mm, ACN/Water (0.1% FA)=5%-5%-95%-95%-95%-5%, 0-0.5 min-10 min-10.5 min-12.0 min).

### Compound 302

### N1-(4-(tert-butyl)-3-(4-methoxybutoxy) phenyl)cyclopentane-1,3-diamine

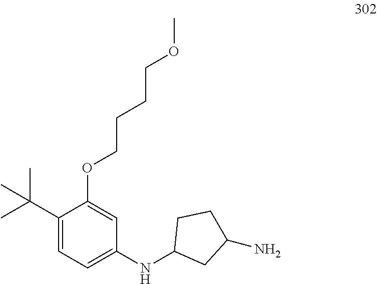 [SMILES: COCCCCOc1cc(NC2CCC(N)C2)ccc1C(C)(C)C]

The title compound 302 (12.5 mg) was prepared in a total yield of 28.2% as a yellow solid with 7:3 mixture by ¹H NMR from tert-butyl (3-((4-(tert-butyl)-3-(4-methoxybutoxy) phenyl)amino)cyclopentyl)carbamate (58 mg, 0.13 mmol) and TFA (2.0 mL) according to the procedure for 254. ¹H NMR (400 MHZ, Methanol-d₄) δ 7.00 (dd, J=8.4, 4.1 Hz, 1H), 6.29 (d, J=11.8 Hz, 1H), 6.18 (t, J=10.5 Hz, 1H), 4.02-3.97 (m, 0.7H), 3.97-3.92 (m, 2H), 3.87-3.83 (m, 2H), 3.76-3.68 (m, 0.7H), 3.64-3.59 (m, 0.3H), 3.46 (t, J=6.2 Hz, 2H), 3.33 (s, 3H), 2.55-1.77 (m, 10H), 1.69-1.57 (m, 2H), 1.30 (s, 9H). Mass (m/z): 335.3 [M+H]⁺.

### Compound 303

### N-(4-(4-aminobutyl)cyclohexyl)-4-(tert-butyl)aniline

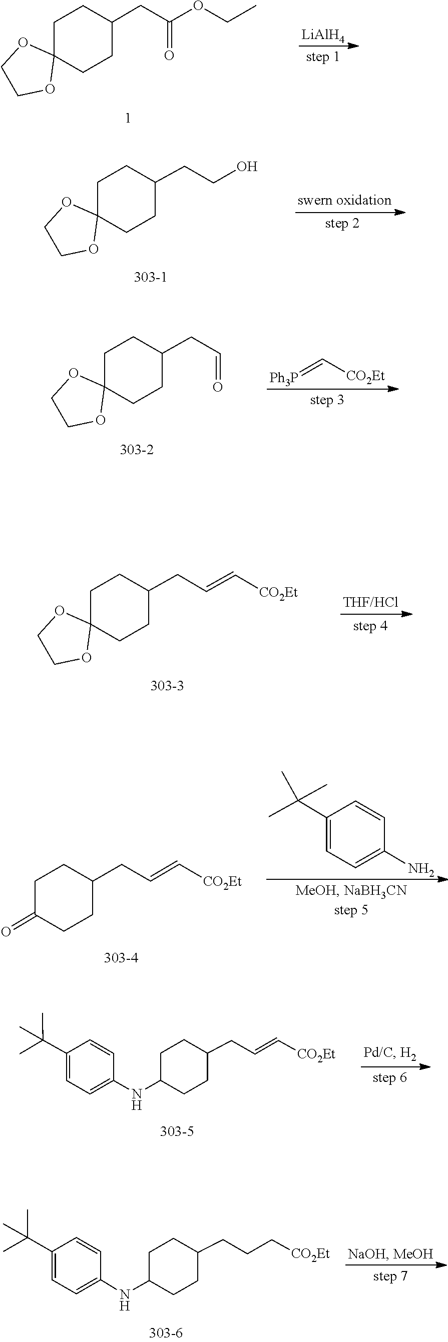

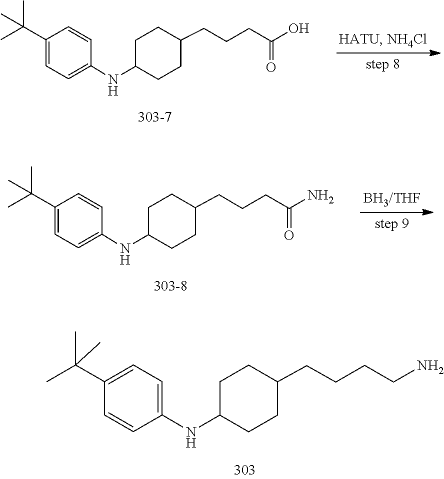 [SMILES: CC(C)(C)c1ccc(NC2CCC(CCCC(=O)O)CC2)cc1 ; CC(C)(C)c1ccc(NC2CCC(CCCC(N)=O)CC2)cc1 ; CC(C)(C)c1ccc(NC2CCC(CCCCN)CC2)cc1]

Step 1. Preparation of 2-(1,4-dioxaspiro[4.5]decan-8-yl) ethan-1-ol (303-1)

To a solution of ethyl 2-(1,4-dioxaspiro[4.5]decan-8-yl) acetate (2 g, 8.76 mmol) in THF (30 mL) stirred 0° C. was added LiAlH₄ (998 mg, 26.28 mmol) in 3 min. 1 mL of H₂O was added into the mixture after stirred at 25° C. for 16 hrs. The mixture was filtered, diluted with H₂O, extracted with EA, dried over Na₂SO₄, filtered and evaporated to give the product (1.5 g, 92%). ¹H NMR (400 MHZ, CDCl₃) δ 3.94 (s, 4H), 3.70 (t, J=6.6 Hz, 2H), 1.75 (d, J=9.8 Hz, 4H), 1.62-1.40 (m, 7H), 1.27 (dd, J=22.2, 11.3 Hz, 2H).

Step 2. Preparation of 2-(1,4-dioxaspiro[4.5]decan-8-yl) acetaldehyde (303-2)

To a solution of oxalyl dichloride (1.23 g, 9.66 mmol) in THF (20 mL) was cooled to −78° C. was added DMSO (1.05 g, 13.42 mmol) and stirred at −78° C. for 30 min. Then a solution of 2-(1,4-dioxaspiro[4.5]decan-8-yl) ethan-1-ol (1.5 g, 8.05 mmol) was added into the mixture dropwise. After 1.5h stirring, triethylamine (6.52 g, 64.43 mmol) was added dropwise. Then the reaction was allowed to warm to rt and stirred for 12 h. The mixture was filtered, evaporated and the residue was applied on silica gel column with EA/PE (0:1-1:5) to afford the product (930 mg, 94%). ¹H NMR (301 MHZ, CDCl₃) δ 9.77 (s, 1H), 3.94 (s, 4H), 2.36 (d, J=6.6 Hz, 2H), 1.94 (s, 1H), 1.75 (d, J=11.1 Hz, 4H), 1.58 (t, J=12.2 Hz, 2H), 1.43-1.18 (m, 2H).

Step 3. Preparation of ethyl (E)-4-(1,4-dioxaspiro[4.5]decan-8-yl) but-2-enoate (303-3)

To a solution of 2-(1,4-dioxaspiro[4.5]decan-8-yl) acetaldehyde (800 mg, 4.34 mmol) in Tol (20 mL) stirred at 80° C. was added DBU (4.54 g, 13.03 mmol) and ethyl 2-(triphenyl-15-phosphanylidene) acetate (1.98 g, 13.03 mmol). The reaction mixture was stirred at 80° C. for 16 h. The residue was applied on to silica gel column with EA: PE (0:1-1:5) to afford the product as yellow oil (800 mg, 72.4%). Mass (m/z): 254.7 [M+H]

Step 4. Preparation of ethyl (E)-4-(4-oxocyclohexyl)but-2-enoate (303-4)

To a solution of (E)-4-(1,4-dioxaspiro[4.5]decan-8-yl) but-2-enoate (800 mg, 3.15 mmol) in THF (20 mL) was added 2N HCl (10 mL). Then the mixture was stirred 16 hours at rt. The reaction was concentrated under vacuum. The residue was applied on to silica gel column with EA: PE (0:1-1:5) to afford the desired product as a colorless oil. (615 mg, 92.98%). ¹H NMR (300 MHz, CDCl₃) δ 7.03-6.87 (m, 1H), 5.85 (d, J=15.5 Hz, 1H), 4.14 (dq, J=21.0, 6.9 Hz, 2H), 2.49-2.29 (m, 4H), 2.23 (t, J=7.0 Hz, 2H), 2.14-2.00 (m, 2H), 2.00-1.81 (m, 1H), 1.53-1.36 (m, 2H), 1.29 (t, J=7.1 Hz, 3H).

Step 5. Preparation of ethyl (E)-4-(4-((4-(tert-butyl)phenyl)amino)cyclohexyl)but-2-enoate (303-5)

To a solution of ethyl (E)-4-(4-oxocyclohexyl)but-2-cnoatc (615 mg, 2.92 mmol) and 4-(tert-butyl)aniline (524 mg, 3.51 mmol) in THF (20 mL) was stirred at 50° C. for 2 hours. Then the mixture was cooled to rt and NaBH₃CN (551 mg, 8.77 mmol) was added. The mixture was stirred 16 hours at rt. The reaction was concentrated under vacuum. The residue was applied on to silica gel column with EA: PE (0:1-1:5) to afford the desired product as colorless oil (530 mg, 52.7%). Mass (m/z): 343.7 [M+H]⁺.

Step 6. Preparation of ethyl 4-(4-((4-(tert-butyl)phenyl)amino)cyclohexyl)butanoate (303-6)

To a solution of ethyl (E)-4-(4-((4-(tert-butyl)phenyl)amino)cyclohexyl)but-2-enoate (530 mg, 1.54 mmol) in THF (20 ml) was added Pd/C (50 mg). Then the mixture was stirred 16 hours at rt under H₂ atmosphere. The crude mixture was filtered, concentrated under vacuum to afford the desired product as a colorless oil. (520 mg, 97.5%). Mass (m/z): 345.7 [M−H]⁺.

Step 7. Preparation of 4-(4-((4-(tert-butyl)phenyl)amino)cyclohexyl)butanoic acid (303-7)

To a solution of ethyl 4-(4-((4-(tert-butyl)phenyl)amino)cyclohexyl)butanoate (520 mg, 1.50 mmol) in McOH (15 mL) was added 4 N NaOH (1.1 mL). Then the mixture was stirred 16 hours at 50° C. The reaction was adjusted to pH 4 with 2 N HCl, exacted with EA (30 mL×3), dried over Na₂SO₄, filtered, concentrated under vacuum. The residue was used directly as a colorless oil (455 mg, 95.2%).

Step 8. Preparation of 4-(4-((4-(tert-butyl)phenyl)amino)cyclohexyl)butanamide (303-8)

To a solution of 4-(4-((4-(tert-butyl)phenyl)amino)cyclohexyl)butanoic acid (120 mg, 0.378 mmol), NH₄Cl (61 mg, 1.134 mmol), HATU (287 mg, 0.756 mmol) and TEA (115 mg, 1.134 mmol) in DCM (10 mL) stirred 2 hours at rt. The reaction was washed with water, exacted with EA (20 mL×3), dried over Na₂SO₄, filtered and concentrated under vacuum. The residue was applied on to silica gel column with EA: PE (0:1-2:1) to afford the desired product (100 mg, 83.5%) as a white solid. Mass (m/z): 316.7 [M+H]⁺.

Step 9. Preparation of ethyl N-(4-(4-aminobutyl)cyclohexyl)-4-(tert-butyl)aniline (303)

4-(4-((4-(tert-Butyl)phenyl)amino)cyclohexyl)butanamide (100 mg, 0.32 mmol) in BH₃-THF (30 mL) was stirred at 50° C. for 16 hours. The mixture was quenched with methanol, evaporated and purified by prep-HPLC to afford the product (29.2 mg, 30.2%) as a white solid. ¹H NMR (400 MHZ, Methanol-d₄) δ 7.16-7.11 (m, 2H), 6.63-6.58 (m, 2H), 3.17-3.07 (m, 1H), 2.93-2.84 (m, 2H), 1.71-1.51 (m, 6H), 1.44-1 32 (m, 6H), 1.23 (s, 9H), 1.16-0.99 (m, 3H). Mass (m/z): 303.2 [M+H]⁺.

### Compound 304

### N1-(4-ethyl-3-methylphenyl)cyclohexane-1,4-diamine

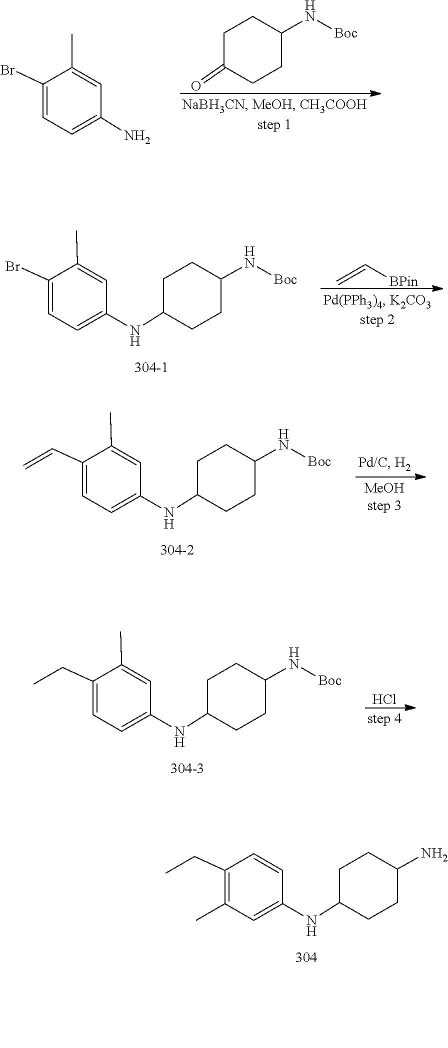

Step 1. Preparation of tert-butyl (4-((6-ethylpyridin-3-yl)amino)cyclohexyl)carbamate (304-1) To a solution of 4-bromo-3-methylaniline (1 g, 5.4 mmol) in MeOH (10 mL) and a drop of AcOH was added tert-butyl (4-oxocyclohexyl)carbamate (1.4 g, 6.5 mmol). Then the mixture was added NaBH₃CN (680 mg, 10.8 mmol) and the mixture was stirred at 25° C. for 16 h. LCMS showed the reaction was completed. The reaction was quenched with water (10 mL), extracted with EA (10 mL*3). The combined organic layers were washed with brine (20 mL), dried over sodium sulfate, filtered and concentrated. The residue was purified by combi-flash with EA/PE (1:3) to afford to give 304-1 (1.5 g, 72.2% yield) as a yellow solid. MS (m/z) 383.1 [M+H]⁺.

Step 2. Preparation of tert-butyl (4-((3-methyl-4-vinylphenyl)amino)cyclohexyl)carbamate (304-2)

To a solution of 304-1 (400 mg, 1.04 mmol), 4,4,5,5-tetramethyl-2-vinyl-1,3,2-dioxaborolane (240 mg, 1.56 mmol), K₂CO₃ (430 mg, 3.12 mmol) in 1,4-dioxane (20 mL) and water (2 mL) was added Pd(PPh₃)₄ (120 mg, 0.10 mmol). The mixture was stirred with refluxing overnight under N₂ atmosphere. After cooling to ambient temperature, concentrated under reduced pressure. The residue was purified by silica gel column to provide 304-2 (240 mg, 69.5% yield) as a yellow solid. MS (m/z) 330.9 [M+H]⁺.

Step 3. Preparation of tert-butyl (4-((4-ethyl-3-methylphenyl)amino)cyclohexyl)carbamate (304-3)

Pd/C to a solution (50 mg, 10%) was added of 2-(4,4-dimethylcyclohex-1-en-1-yl)-5-nitropyridine (240 mg, 0.72 mmol) in MeOH (10 mL), the mixture was allowed to react under H₂ for 16 h, then filtered and solvent was removed under vacuum to give 304-3 (140 mg, 57.9% yield) as a yellow solid. MS (m/z) 332.9 [M+H]⁺.

Step 4. Preparation of N1-(4-ethyl-3-methylphenyl)cyclohexane-1,4-diamine (304)

304-3 (140 mg, 0.42 mmol) and HCl in 1,4-dioxane (10 mL, 4N) were placed in a flask stirred at 25° C. for 16 hrs. Excess 1,4-dioxane. was distilled under vacuum and the residue was purified by prep-HPLC (column-Xbridge-C_{18 150×19} mm, 5 μm; Mobile phase: ACN-H₂O (0.1% FA), 20%-40%) to afford 304 (36 mg 36.9%) as a white solid. ¹H NMR (400 MHz, CD₃OD) δ 7.28 (dd, J=23.3, 8.0 Hz, 1H), 7.14 (d, J=9.3 Hz, 1H), 7.07 (d, J=9.2 Hz, 1H), 3.65-3.37 (m, 1H), 3.24-3.06 (m, 1H), 2.65 (dd, J=9.6, 7.6 Hz, 2H), 2.34 (d, J=9.4 Hz, 3H), 2.14-2.07 (m, 2H), 1.89 (dd, J=10.6, 6.6 Hz, 4H), 1.58-1.44 (m, 2H), 1.18 (td, J=7.6, 3.7 Hz, 3H). MS (m/z) 233.2 [M+H]⁺.

### Compound 305

### N1-(5,6,7,8-tetrahydronaphthalen-2-yl)cyclohexane-1,4-diamine

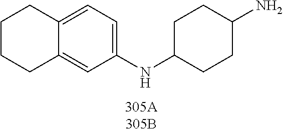 [SMILES: NC1CCC(Nc2ccc3c(c2)CCCC3)CC1]

The title compound 305 was prepared from tert-butyl (4-((5,6,7,8-tetrahydronaphthalen-2-yl)amino)cyclohexyl)carbamate (88 mg, 0.256 mmol), TFA (1 mL), and DCM (10 mL) according to the procedure for 24, which was purified by prep HPLC (Column: X Select-CSH-Prep 5 μm OBD, 19*150 mm; ACN/water (0.5% TFA)=10%-25%-85%-95%-15%, 0 min-10 min-10.5 min-11.5 min-13 min) to give the desired product 305A (Rt=5.9 min) as a white solid (2.7 mg, 4.3%) and 305B (Rt=7.0 min) as yellow oil (8.9 mg, 14.2%). 305A: ¹H NMR (400 MHZ, DMSO-d₆) δ 6.74 (d, J=8.0 Hz, 1H), 6.46-6.20 (m, 2H), 5.03 (s, 1H), 3.00 (d, J=54.8 Hz, 2H), 2.57 (d, J=18.8 Hz, 4H), 1.99 (d, J=11.6 Hz, 2H), 1.75 (d, J=15.2 Hz, 3H), 1.66 (p, J=3.2 Hz, 4H), 1.59 (d, J=12.4 Hz, 1H), 1.46 (q, J=12.0 Hz, 1H), 1.18 (t, J=20.0 Hz, 1H). Mass (m/z): 245.3 [M+H]⁺. 305B: ¹H NMR (400 MHZ, DMSO-d₆) δ 6.74 (d, J=8.0 Hz, 1H), 6.46-6.20 (m, 2H), 5.03 (s, 1H), 3.00 (d, J=54.8 Hz, 2H), 2.57 (d, J=18.8 Hz, 4H), 1.99 (d, J=11.6 Hz, 2H), 1.75 (d, J=15.2 Hz, 3H), 1.66 (p, J=3.2 Hz, 4H), 1.59 (d, J=12.4 Hz, 1H), 1.46 (q, J=12.0 Hz, 1H), 1.18 (t, J=20.0 Hz, 1H). Mass (m/z): 245.3 [M+H]⁺.

### Compound 306

### N1-(3,4-dimethylphenyl)cyclohexane-1,4-diamine

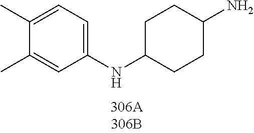 [SMILES: Cc1ccc(NC2CCC(N)CC2)cc1C]

The title compound 306 was prepared from tert-butyl (4-((3,4-dimethylphenyl)amino)cyclohexyl)carbamate (128 mg, 0.402 mmol), TFA (1 mL), and DCM (10 mL) according to the procedure for 24, which was purified by prep HPLC (solvent system (Column: X Select-CSH-Prep 5 μm OBD, 19*150 mm; ACN/water (0.5% TFA)=15%-25%-95%-95%-15%, 0 min-9 min-9.5 min-10.5 min-13 min) to give the desired product 306A (Rt=6.6 min) as a white solid (4.8 mg, 5.4%) and 306B (Rt=7.5 min) as a white solid (5.5 mg, 6.3%). 306A: ¹H NMR (400 MHZ, DMSO-d₆) δ 6.76 (dd, J=8.0, 5.2 Hz, 1H), 6.36 (dd, J=18.4, 2.4 Hz, 1H), 6.27 (ddd, J=15.6, 8.0, 2.4 Hz, 1H), 4.98 (d, J=7.6 Hz, 1H), 3.37-2.75 (m, 2H), 2.05 (d, J=2.4 Hz, 3H), 2.00 (d, J=2.4 Hz, 3H), 1.97-1.86 (m, 2H), 1.73-1.47 (m, 4H), 1.36 (qd, J=13.6, 12.4, 3.6 Hz, 1H), 1.15-1.02 (m, 1H). Mass (m/z): 219.3 [M+H]⁺. 306B: ¹H NMR (400 MHZ, DMSO-d₆) δ 6.76 (dd, J=8.0, 5.2 Hz, 1H), 6.36 (dd, J=18.4, 2.4 Hz, 1H), 6.27 (ddd, J=15.6, 8.0, 2.4 Hz, 1H), 4.98 (d, J=7.6 Hz, 1H), 3.37-2.75 (m, 2H), 2.05 (d, J=2.4 Hz, 3H), 2.00 (d, J=2.4 Hz, 3H), 1.97-1.86 (m, 2H), 1.73-1.47 (m, 4H), 1.36 (qd, J=13.6, 12.4, 3.6 Hz, 1H), 1.15-1.02 (m, 1H). Mass (m/z): 219.3 [M+H]⁺.

### Compound 307

### N-(((1s,4s)-4-aminocyclohexyl)methyl)-2,3-dihydro-1H-inden-5-amine

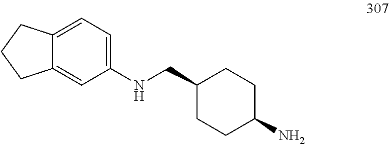 [SMILES: N[C@H]1CC[C@@H](CNc2ccc3c(c2)CCC3)CC1]

The title compound 307 (48.2 mg) was prepared in a total yield of 74.7% as a yellow solid from tert-butyl ((1s,4s)-4-((E)-((2,3-dihydro-1H-inden-5-yl) imino) methyl)cyclohexyl)carbamate (91 mg, 0.264 mmol), TFA (1 mL), and DCM (10 mL) according to the procedure for 24. ¹H NMR (400 MHz, DMSO-d₆) δ 6.88 (d, J=8.0 Hz, 1H), 6.49-6.29 (m, 2H), 5.43 (dt, J=21.6, 5.6 Hz, 1H), 3.15 (tt, J=7.2, 3.2 Hz, 1H), 2.86 (dt, J=46.0, 5.6 Hz, 2H), 2.70 (dt, J=14.8, 7.2 Hz, 4H), 2.03-1.84 (m, 3H), 1.78-1.44 (m, 7H), 1.41-1.26 (m, 1H). Mass (m/z): 245.3 [M+H]⁺.

### Compound 308

### N-(((1r,4r)-4-aminocyclohexyl)methyl)-2,3-dihydro-1H-inden-5-amine (308)

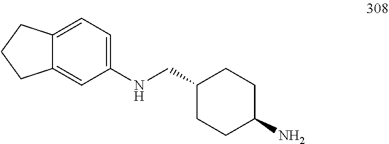 [SMILES: N[C@H]1CC[C@H](CNc2ccc3c(c2)CCC3)CC1]

The title compound 308 (43.9 mg) was prepared in a total yield of 54.9% as a yellow solid from tert-butyl ((1r,4r)-4-((E)-((2,3-dihydro-1H-inden-5-yl) imino) methyl)cyclohexyl)carbamatetert-butyl (113 mg, 0.328 mmol), TFA (1 mL), and DCM (10 mL) according to the procedure for 24. ¹H NMR (400 MHZ, DMSO-d₆) δ 6.89 (d, J=8.0 Hz, 1H), 6.42 (d, J=2.4 Hz, 1H), 6.33 (dd, J=8.0, 2.0 Hz, 1H), 5.39 (q, J=6.0 Hz, 1H), 2.91 (td, J=12.0, 10.0, 6.0 Hz, 1H), 2.81 (t, J=5.6 Hz, 2H), 2.70 (dt, J=14.8, 7.4 Hz, 4H), 2.02-1.90 (m, 4H), 1.90-1.81 (m, 2H), 1.72-1.40 (m, 2H), 1.36-1.19 (m, 2H), 0.98 (qd, J=13.2, 3.1 Hz, 2H). Mass (m/z): 245.3 [M+H]⁺.

### Compound 309

### N-(((1r,4r)-4-aminocyclohexyl)methyl)-5,6,7,8-tetrahydronaphthalen-2-amine

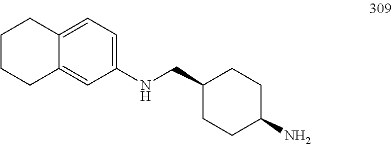 [SMILES: N[C@H]1CC[C@@H](CNc2ccc3c(c2)CCCC3)CC1]

The title compound 309 (15.1 mg) was prepared in a total yield of 27.8% as a yellow solid from tert-butyl tert-butyl ((1s,4s)-4-((E)-((5,6,7,8-tetrahydronaphthalen-2-yl) imino) methyl)cyclohexyl)carbamate (75 mg, 0.209 mmol), TFA (1 mL), and DCM (10 mL) according to the procedure for 24. ¹H NMR (400 MHZ, DMSO-d₆) δ 6.72 (d, J=8.2 Hz, 1H), 6.38-6.27 (m, 1H), 6.22 (dd, J=8.2, 2.4 Hz, 1H), 5.32 (dt, J=17.5, 5.8 Hz, 1H), 3.14 (tt, J=7.2, 3.3 Hz, 1H), 2.84 (dt, J=44.0, 6.1 Hz, 2H), 2.57 (d, J=5.1 Hz, 4H), 1.96 (td, J=12.3, 11.4, 5.7 Hz, 1H), 1.84 (d, J=13.4 Hz, 1H), 1.75-1.54 (m, 9H), 1.51 (q, J=4.4, 4.0 Hz, 1H), 1.30 (tt, J=12.7, 6.7 Hz, 1H), 1.10-0.89 (m, 1H). Mass (m/z): 259.3 [M+H]⁺.

### Compound 310

### N-(((1r,4r)-4-aminocyclohexyl)methyl)-5,6,7,8-tetrahydronaphthalen-2-amine

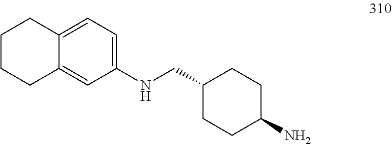 [SMILES: N[C@H]1CC[C@H](CNc2ccc3c(c2)CCCC3)CC1]

The title compound 310 (37 mg) was prepared in a total yield of 27.8% as a yellow solid from tert-butyl tert-butyl ((1s,4s)-4-((E)-((5,6,7,8-tetrahydronaphthalen-2-yl) imino) methyl)cyclohexyl)carbamate (82 mg, 0.229 mmol), TFA (1 mL), and DCM (10 mL) according to the procedure for 24. ¹H NMR (400 MHZ, DMSO-d₆) δ 6.70 (d, J=8.4 Hz, 1H), 6.31 (dd, J=8.4, 2.4 Hz, 1H), 6.20 (d, J=2.4 Hz, 1H), 5.30 (s, 1H), 2.87 (ddt, J=12.0, 7.6, 4.0 Hz, 1H), 2.77 (d, J=6.4 Hz, 2H), 2.56 (dd, J=14.0, 8.8 Hz, 4H), 1.98 (dd, J=12.8, 4.0 Hz, 2H), 1.89-1.77 (m, 2H), 1.64 (h, J=4.0, 3.6 Hz, 4H), 1.52-1.40 (m, 1H), 1.32 (qd, J=12.4, 3.2 Hz, 2H), 0.96 (qd, J=13.2, 3.1 Hz, 2H). Mass (m/z): 259.3 [M+H]⁺.

### Compound 311

### N1-(4-propoxyphenyl)cyclohexane-1,4-diamine

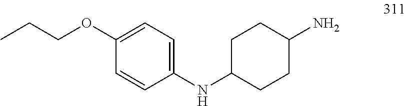 [SMILES: CCCOc1ccc(NC2CCC(N)CC2)cc1]

The title compound 311 (89.7 mg) was prepared in a total yield of 65.2% as a yellow oil with 1:2 mixture by ¹H NMR from tert-butyl (4-((4-propoxyphenyl)amino)cyclohexyl)carbamate (193 mg, 0.554 mmol), TFA (1 mL), and DCM (10 mL) according to the procedure for 24. ¹H NMR (400 MHZ, DMSO-d₆) δ 7.96 (d, J=12.4 Hz, 2H), 5.43 (dd, J=48.0, 7.6 Hz, 1H), 4.05 (td, J=6.8, 1.6 Hz, 2H), 2.43 (d, J=5.6 Hz, 2H), 2.20 (d, J=3.6 Hz, 2H), 1.72 (dd, J=12.8, 4.3 Hz, 4H), 1.63 (d, J=6.4 Hz, 4H), 0.89 (t, J=7.6 Hz, 3H). Mass (m/z): 249.3 [M+H]⁺.

### Compound 312

### N-(4-(aminomethyl)cyclohexyl)-3,4,5-trimethylaniline

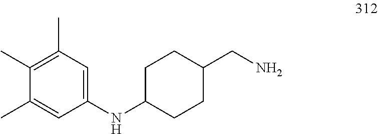 [SMILES: Cc1cc(NC2CCC(CN)CC2)cc(C)c1C]

The title compound 312 (59.6 mg) was prepared in a total yield of 61.2% as a yellow solid with 1:2 mixture by ¹H NMR from tert-butyl ((4-((3,4,5-trimethylphenyl)amino)cyclohexyl)methyl)carbamate (137 mg, 0.369 mmol), TFA (1 mL), and DCM (10 mL) according to the procedure for 24. ¹H NMR (400 MHZ, DMSO-d₆) δ 6.21 (d, J=28.0 Hz, 2H), 4.86 (d, J=30.8 Hz, 1H), 3.16-2.67 (m, 1H), 2.54 (dd, J=26.4, 6.8 Hz, 2H), 2.06 (d, J=2.0 Hz, 6H), 1.91 (d, J=1.2 Hz, 3H), 1.83-1.66 (m, 1H), 1.56 (dq, J=9.2, 4.2 Hz, 2H), 1.51-1.31 (m, 4H). Mass (m/z): 247.3 [M+H]⁺.

### Compound 313

### 1-methyl-N4-(3,4,5-trimethylphenyl)cyclohexane-1,4-diamine

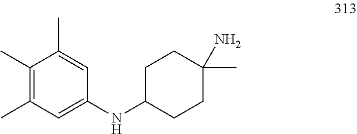

The title compound 313A (13.6 mg) was prepared in a yield of 14.9% as a brown solid by 1H NMR compound 313B (14.9 mg) was prepared in a yield of 16.35% as a brown solid from 3,4,5-trimethylaniline (50 mg, 0.37 mmol) and tert-butyl (1-methyl-4-oxocyclohexyl)carbamate (50 mg, 0.22 mmol) according to the procedure for 20. 313A: ¹H NMR (400 MHZ, DMSO-d₆) δ 8.05 (d, J=31.5 Hz, 2H), 6.26 (d, J=11.9 Hz, 2H), 4.84 (d, J=69.4 Hz, 1H), 3.19 (d, J=42.5 Hz, 1H), 2.11 (s, 6H), 1.96 (s, 3H), 1.92-1.83 (m, 2H), 1.80-1.70 (m, 2H), 1.70-1.61 (m, 1H), 1.55 (t, J=8.4 Hz, 2H), 1.31 (s, 1H), 1.28 (d, J=6.6 Hz, 3H). Mass (m/z): 247.5 [M+H]⁺. 313B: ¹H NMR (400 MHZ, DMSO-d₆) δ 8.08 (s, 2H), 6.25 (s, 1H), 6.22 (s, 1H), 5.02-4.61 (m, 1H), 3.17 (d, J=45.2 Hz, 1H), 2.09 (s, 6H), 1.94 (d, J=2.1 Hz, 3H), 1.89-1.81 (m, 2H), 1.71 (s, 2H), 1.64 (s, 1H), 1.53 (d, J=9.7 Hz, 1H), 1.31-1.20 (m, 5H). Mass (m/z): 247.5 [M+H]⁺.

### Compound 314

### N-(((1r,4r)-4-aminocyclohexyl)methyl)-3,4,5-trimethylaniline

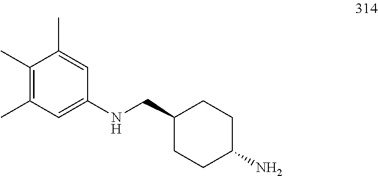 [SMILES: Cc1cc(NC[C@H]2CC[C@H](N)CC2)cc(C)c1C]

The title compound 314 (34.2 mg) was prepared in a yield of 47.3% as a white powder from 3,4,5-trimethylaniline (40 mg, 0.29 mmol) and tert-butyl ((1r,4r)-4-formylcyclohexyl)carbamate (80 mg, 0.35 mmol) according to the procedure for 20. 1¹H NMR (400 MHZ, DMSO-d₆) δ 8.23-7.95 (m, 2H), 6.22 (d, J=8.3 Hz, 2H), 5.18 (s, 1H), 2.90 (d, J=7.4 Hz, 1H), 2.83-2.74 (m, 2H), 2.10 (s, 6H), 1.95 (s, 3H), 1.84 (dd, J=13.9, 3.6 Hz, 2H), 1.64 (d, J=6.9 Hz, 1H), 1.57-1.38 (m, 2H), 1.39-1.19 (m, 2H), 1.06-0.91 (m, 2H). Mass (m/z): 247.2 [M+H]⁺.

### Compound 315

### N-(((1r,4r)-4-aminocyclohexyl)methyl)-4-propylaniline

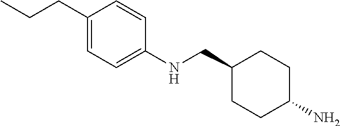 [SMILES: CCCc1ccc(NC[C@H]2CC[C@H](N)CC2)cc1]

The title compound 315 (12.3 mg) was prepared in a yield of 17.02% as a white powder from 4-propylaniline (40 mg, 0.29 mmol) and tert-butyl ((1r,4r)-4-formylcyclohexyl)carbamate (80 mg, 0.35 mmol) according to the procedure for 20. ¹H NMR (400 MHZ, DMSO-d₆) δ 8.01 (d, J=19.6 Hz, 2H), 6.87 (dd, J=8.5, 2.1 Hz, 2H), 6.47 (dd, J=8.9, 7.2 Hz, 2H), 5.43 (s, 1H), 2.93 (q, J=7.8, 6.7 Hz, 1H), 2.81 (t, J=6.2 Hz, 1H), 2.37 (t, J=7.5 Hz, 2H), 2.00-1.81 (m, 3H), 1.66 (s, 1H), 1.61-1.37 (m, 4H), 1.29 (dd, J=26.4, 13.7 Hz, 2H), 1.09-0.95 (m, 2H), 0.86 (t, J=7.3 Hz, 3H). Mass (m/z): 247.6 [M+H]⁺.

### Compound 316

### N-(((1r,4r)-4-aminocyclohexyl)methyl)-3,4-dimethylaniline

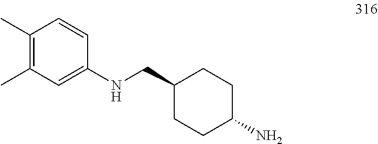 [SMILES: Cc1ccc(NC[C@H]2CC[C@H](N)CC2)cc1C]

The title compound 316 (34.9 mg) was prepared in a yield of 52.0% as a white powder from 3,4-dimethylaniline (35 mg, 0.29 mmol) and tert-butyl ((1r,4r)-4-formylcyclohexyl)carbamate (80 mg, 0.35 mmol) according to the procedure for 20. ¹H NMR (400 MHZ, DMSO-d₆) δ 7.91 (s, 2H), 6.81 (d, J=8.2 Hz, 1H), 6.38 (s, 1H), 6.29 (d, J=8.1 Hz, 1H), 5.29 (s, 1H), 2.81 (d, J=6.6 Hz, 2H), 2.10 (s, 3H), 2.05 (s, 3H), 2.00-1.90 (m, 2H), 1.90-1.81 (m, 2H), 1.64 (t, J=6.2 Hz, 1H), 1.54 (q, J=5.7 Hz, 1H), 1.33-1.23 (m, 2H), 1.06-0.93 (m, 2H). Mass (m/z): 233.2 [M+H]⁺.

### Compound 317

### 3-((4-((4-(tert-butyl)phenyl)amino)cyclohexyl)amino)propan-1-ol

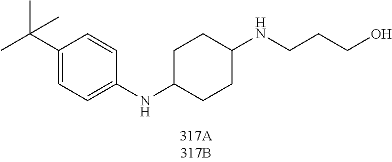 [SMILES: CC(C)(C)c1ccc(NC2CCC(NCCCO)CC2)cc1]

The title compound 317A (21.1 mg) was prepared in a yield of 34.0% as a white powder and compound 317B (22.3 mg) was prepared in a yield of 35.9% as a white powder from 4-((4-(tert-butyl)phenyl)amino)cyclohexan-1-one (50 mg, 0.20 mmol), methyl 3-aminopropan-1-ol (46 mg, 0.61 mmol) and NaBH (OAc) 3 (86 mg, 0.40 mmol) according to the procedure for 20. 317A: ¹H NMR (400 MHZ, DMSO-d₆) δ 8.61 (s, 1H), 7.13-6.99 (m, 2H), 6.59-6.46 (m, 2H), 5.22 (d, J=5.6 Hz, 1H), 4.76 (s, 1H), 3.48 (t, J=6.0 Hz, 2H), 3.46-3.41 (m, 1H), 3.07 (s, 1H), 2.95 (s, 2H), 1.88-1.67 (m, 8H), 1.57 (ddd, J=13.9, 9.5, 3.6 Hz, 2H), 1.20 (s, 9H). Mass (m/z): 305.6 [M+H]⁺. HPLC: Rt=2.070 mins (Agilent, poroshell 120, SB—C18 2.7 μm, 4.6×50 mm, ACN/Water (0.1%)=5%-5%-95%-95%-95%-5%, 0-0.5 min-10 min-10.5 min-12.0 min). 317B: ¹H NMR (400 MHZ, DMSO-d₆) δ 7.12-7.02 (m, 2H), 6.55-6.46 (m, 2H), 5.20 (d, J=8.1 Hz, 1H), 3.49 (t, J=6.0 Hz, 2H), 3.16-3.05 (m, 1H), 2.96 (q, J=7.9 Hz, 3H), 2.15-1.98 (m, 4H), 1.85-1.74 (m, 2H), 1.48 (q, J=12.2, 11.7 Hz, 2H), 1.21 (s, 9H), 1.19-1.12 (m, 2H). Mass (m/z): 305.6 [M+H]⁺. HPLC: Rt=1.944 mins (Agilent, poroshell 120, SB—C_{18 2.7} μm, 4.6×50 mm, ACN/Water (0.1%)-5%-5%-95%-95%-95%-5%, 0-0.5 min-10 min-10.5 min-12.0 min).

### Compound 318

### 1-N-(4-cyclobutylphenyl)cyclohexane-1,4-diamine

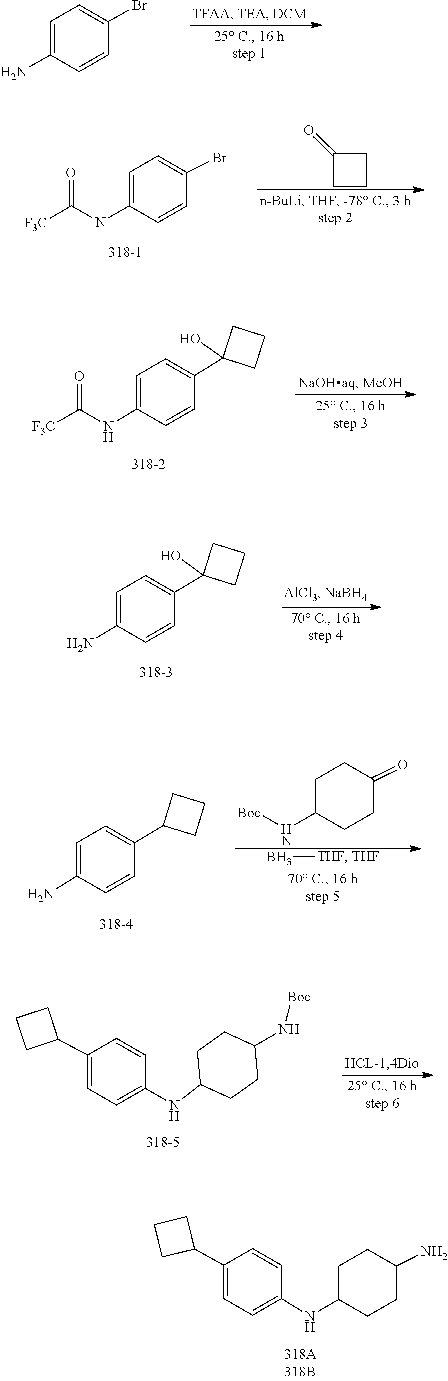

Step 1. N-(4-bromophenyl)-2,2,2-trifluoroacetamide (318-1)

A mixture of 4-bromoaniline (1 g, 5.8 mmol), TFAA (2.35 ml), TEA (1.23 ml) in DCM (10 mL) was stirred overnight at 25° C. The pH of the water phase was adjusted to 8 by 1N NaOH. It was quenched by H₂O (300 mL) and extracted with EA (3×300 mL), dried over Na₂SO₄, concentrated to afford target product (600 mg, yield: 30.7%) as a yellow solid.

Step 2. 2,2,2-trifluoro-N-(4-(1-hydroxycyclobutyl)phenyl) acetamide (318-2)

To a solution of 318-1 (600 mg, 2.23 mmol), cyclobutanone (156.3 mg, 2.23 mmol) in THE (10 ml) was added n-BuLi (5.3 mL) at −78° C. Then the mixture was stirred overnight at rt. The reaction was quenched by adding aq. NH₄Cl. It was quenched by H₂O (100 mL) and extracted with DCM (3×100 mL). The organic layers were combined, washed with brine NaCl (2×100 mL) and dried with MgSO₄, filtered and concentrated. The crude product was applied onto a silica gel column (12 g) eluted with PE: EA (5:1) to give product (400 mg, yield: 90%) as a yellow solid. Mass (m/z): 241.9/262.9 [M+H]⁺.

Step 3. 1-(4-aminophenyl)cyclobutan-1-ol (318-3)

To a solution of 318-2 (400 mg, 1.53 mmol) in MeOH (5 ml) was added 1N NaOH (10 mL) at° C. Then the mixture was stirred overnight at rt. The reaction was concentrated under vacuum. It was quenched by H₂O (100 mL) and extracted with DCM (3×100 mL), dried with MgSO₄, filtered and concentrated to afford target product (200 mg, yield: 63.8%) as a yellow solid. Mass (m/z): 164.2 [M+H]⁺.

Step 4. 4-cyclobutylaniline (318-4)

To a solution of 318-3 (200 mg, 1.22 mmol) in THF (5 mL) was added NaBH₄ (254.96 mg, 6.74 mmol) at 0° C. Then AlCl₃ (490.18 mg, 3.67 mmol) was added in portions. Then the mixture was stirred overnight at rt. The reaction was concentrated under vacuum. It was quenched by H₂O (100 mL) and extracted with EA (3×100 mL). The crude product was applied onto a silica gel column (12 g) eluted with PE: EA (5:1) to give the product (200 mg, yield: 63.8%) as a yellow solid. Mass (m/z): 148.2 [M+H]⁺.

Step 5. tert-butyl (4-((4-cyclobutylphenyl)amino)cyclohexyl)carbamate (318-5)

To a solution of 318-4 (80 mg, 0.31 mmol), Pic-BH₃ (87.22 mg, 0.81 mmol) in AcOH/H₂O=1:10 (5.5 mL) was stirred overnight at 25° C. The pH of the water phase was adjusted to 8 by aq. NaHCO₃. It was quenched by H₂O (30 mL) and extracted with EA (3×30 mL), dried with MgSO₄, filtered and concentrated to afford the target product (100 mg, yield: 17.2%) as a yellow solid. Mass (m/z): 345.3 [M+H]⁺.

Step 6. Preparation of N1-(4-cyclobutylphenyl)cyclohexane-1,4-diamine (318)

To a solution of 318-5 (200 mg, 0.57 mmol) in 1,4-dioxane (5 ml) was added HCl/1,4-dioxane (10 mL). Then the mixture was stirred overnight at 25° C. The reaction was concentrated under vacuum. The residue was purified by prep-TLC to afford the desired products 318A (5.4 mg) as a white solid and 318B (17.8 mg) as a white solid. 318A: ¹H NMR (400 MHZ, CD₃OD) δ 7.01-6.94 (m, 2H), 6.64-6.52 (m, 2H), 3.47-3.31 (m, 2H), 3.27-3.15 (m, 0.5H), 3.10-3.02 (m, 0.5H), 2.29-2.20 (m, 2H), 2.17-2.10 (m, 2H), 2.09-1.96 (m, 5H), 1.83-1.75 (m, 1H), 1.49 (qd, J=13.0, 3.6 Hz, 2H), 1.34-1.17 (m, 2H). Mass (m/z): 452.3 [M+H]⁺. HPLC: Rt=3.582 min (Column: XBRIDGE 3.5 μm, 2.1*50 mm, Mobile phase: H₂O (0.05% TFA)-ACN (0.05% TFA), ACN from 0 to 60% over 7 minutes, 7-8 min, ACN from 60% to 100%, Flow rate: 0.8 mL/min). 318B: ¹H NMR (400 MHZ, CD₃OD) δ 7.06-6.91 (m, 2H), 6.67-6.52 (m, 2H), 3.52 (s, 1H), 3.44-3.32 (m, 1H), 3.25-3.09 (m, 1H), 2.29-2.16 (m, 2H), 2.10-1.91 (m, 3H), 1.90-1.58 (m, 9H). Mass (m/z): 452.3 [M+H]⁺. HPLC: Rt=3.757 min (Column: XBRIDGE 3.5 μm, 2.1*50 mm, Mobile phase: H₂O (0.05% TFA)-ACN (0.05% TFA), ACN from 0 to 60% over 7 minutes, 7-8 min, ACN from 60% to 100%, Flow rate: 0.8 mL/min).

### Compound 319

### 4-(tert-butyl)-N-(4-(2-(dimethylamino) ethoxy) cyclohexyl)aniline

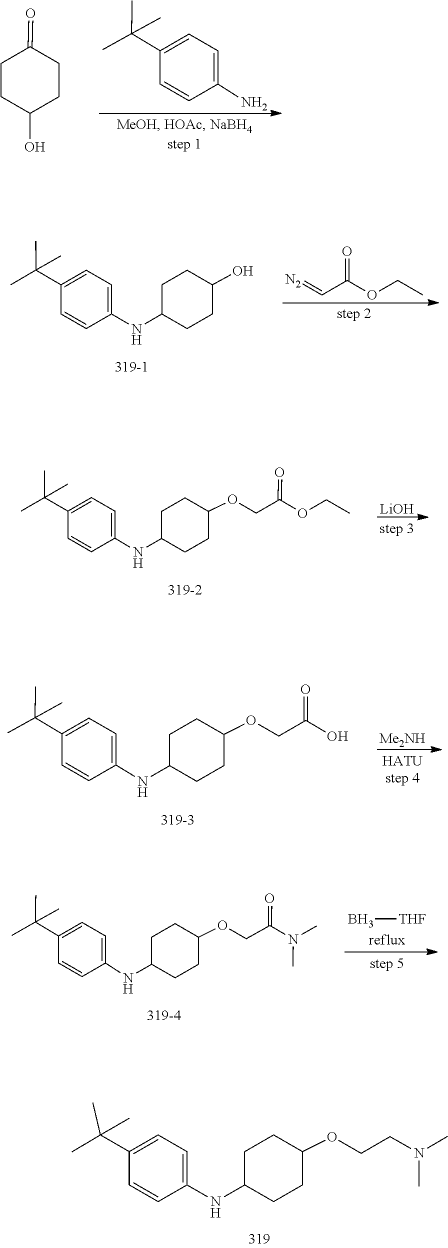

Step 1. Preparation of 4-((4-(tert-butyl)phenyl)amino)cyclohexan-1-ol (319-1)

To a solution of 4-hydroxycyclohexan-1-one (2000 mg, 17.52 mmol) in MeOH (50 mL) and a drop of AcOH was added 4-(tert-butyl)aniline (3138 mg, 21.03 mmol) and the mixture was stirred at 50° C. for 1 h. Then the mixture was added NaBH₄ (1325 mg, 35.04 mmol) after cooling to 25° C. Then the mixture was stirred at 25° C. for 16 hrs. LCMS showed the reaction was completed. The reaction was quenched with water (10 mL), extracted with EA (10 mL*3). The combined organic layers were washed with brine (20 mL), dried over sodium sulfate, filtered and concentrated. The residue was purified by combi-flash with EA/PE (1:3) to afford to give 319-1 (1500 mg, 34.6% yield) as a yellow solid. MS (m/z) 247.9 [M+H]⁺.

Step 2. ethyl 2-((4-((4-(tert-butyl)phenyl)amino)cyclohexyl) oxy) acetate (319-2)

Ethyl 2-diazoacetate (276.7 mg, 2.43 mmol), Rhodium (II) acetate dimer (89.34 mg, 0.20 mmol) was added to a solution of 4-[(4-tert-butylphenyl)amino]cyclohexan-1-ol (500 mg, 2.02 mmol) in DCM (10 mL), the mixture was removed under vacuum. The residue was purified through flash column chromatography to give 319-2 (400 mg, 59.3% yield) as a yellow solid. MS (m/z): 333.8 [M+H]⁺.

Step 3. Preparation of 2-((4-((4-(tert-butyl)phenyl)amino)cyclohexyl) oxy) acetic acid (319-3)

To a solution of 319-2 (400 mg, 1.20 mmol) in THF (4 mL) and water (4 mL) was added LiOH (172.3 mg, 7.2 mmol) and the mixture was stirred at 25° C. for 2 h. the solvent was removed under vacuum. Dissolved in water (20 mL) and adjusted pH=2 with 1NHCl, extracted with EA (20 mL*3). The combined organic layers were washed with brine (20 mL), dried over sodium sulfate, filtered and concentrated to give 319-3 (200 mg, 54.6% yield) as a yellow solid. MS (m/z) 306.2 [M+H]⁺.

Step of 4. Preparation

2-((4-((4-(tert-butyl)phenyl)amino)cyclohexyl) oxy)-N,N-dimethylacetamide (319-4) To a solution of 2-((4-((4-(tert-butyl)phenyl)amino)cyclohexyl) oxy) acetic acid (200 mg, 0.66 mmol) in DMF (5 mL) was added 2N Me₂NH (0.7 mL, 1.31 mmol), triethylamine (9198.8 mg, 1.96 mmol) and HATU (373.5 mg, 0.98 mmol). Then the mixture was stirred at rt. for 2 h. The reaction was extracted by EA (10 mL) for 3 times. The organic layers were dried over sodium sulfate, concentrated under vacuum and the residue was purified by flash to afford the desired product (100 mg, 45.9%) as yellow oil. MS (m/z) 332.9 [M+H]⁺.

Step 5. Preparation of 4-(tert-butyl)-N-(4-(2-(dimethylamino) ethoxy) cyclohexyl)aniline (319)

To a solution of 2-((4-((4-(tert-butyl)phenyl)amino)cyclohexyl) oxy)-N,N-dimethylacetamide (70 mg, 0.21 mmol) in THF (2 mL) was added LiAlH₄ (16.8 mg, 0.42 mmol) and the mixture was stirred at 50° C. for 16 h. The reaction was quenched with water (1 mL), 1N NaOH (1 mL) and water (3 mL). The residue was filtered and the filtrate was removed under vacuum and the residue was purified by prep-HPLC (column-Xbridge-C18 150×21.2 mm, 5 μm; Mobile phase: ACN-H₂O (0.1% FA), 40%-60%) to afford 319A (10.4 mg) as a white solid and 319B (15.5 mg) as a white solid. 319A: ¹H NMR (400 MHz, CD₃OD) δ 7.61 (d, J=8.4 Hz, 2H), 7.35 (d, J=8.5 Hz, 2H), 3.83-3.79 (m, 2H), 3.46 (d, J=3.8 Hz, 1H), 2.91 (s, 6H), 2.68 (s, 4H), 2.20 (d, J=10.2 Hz, 2H), 2.09 (s, 1H), 1.55 (d, J=12.5 Hz, 2H), 1.36 (s, 9H). MS (m/z) 319.3 [M+H]⁺. HPLC: Rt: 3.843 min (Column: XBRIDGE 2.1*50 mm, 3.5 μm; Mobile Phase: H₂O (0.05% TFA)/ACN (0.05% TFA), ACN from 0% to 60% over 7 minutes, 7-8 min, ACN from 60% to 100%; 0.8 mL/min). 319B: ¹H NMR (400 MHZ, CD₃OD) δ 7.51 (d, J-8.6 Hz, 2H), 7.27 (d, J=8.6 Hz, 2H), 3.68-3.64 (m, 2H), 3.58 (s, 1H), 3.43-3.36 (m, 1H), 3.30-3.26 (m, 2H), 2.85 (s, 6H), 1.99 (d, J=15.0 Hz, 2H), 1.78-1.66 (m, 4H), 1.46 (d, J=16.7 Hz, 2H), 1.25 (s, 9H). MS (m/z) 319.3 [M−H]⁺ HPLC: Rt: 3.977 min (Column: XBRIDGE 2.1*50 mm, 3.5 μm; Mobile Phase: H₂O (0.05% TFA)/ACN (0.05% TFA), ACN from 0% to 60% over 7 minutes, 7-8 min, ACN from 60% to 100%; 0.8 mL/min).

### Compound 320

### N1-(4-(1-ethoxy-2-methylpropan-2-yl)phenyl)cyclohexane-1,4-diamine

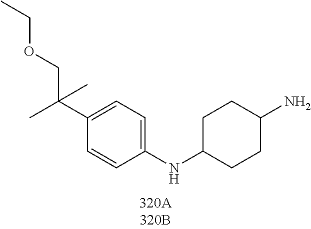 [SMILES: CCOCC(C)(C)c1ccc(NC2CCC(N)CC2)cc1]

The title compound 320 was prepared from tert-butyl (4-((4-(1-ethoxy-2-methylpropan-2-yl)phenyl)amino)cyclohexyl)carbamate (120 mg. 0.3 mmol) according to the procedure for compound 24. The mixture was purified by preparative HPLC (Column: X Select-CSH-Prep 5 μm OBD, 19*150 mm; ACN/water (0.5% TFA)=15%-33%-95%-95%-10%, 0 min-10 min-10.5 min-11.5 min-13.0 min) to afford compound 320A (Rt=7.46 min) as a white solid and compound 320B (Rt=9.14 min) as a white solid. 320A (33.1 mg, 38%): ¹H NMR (400 MHZ, DMSO-d₆) δ 8.20 (s, 2H), 7.06 (d, J=8.4 Hz, 2H), 6.53 (d, J=8.4 Hz, 2H), 5.25 (s, 1H), 3.33 (m, 2H), 3.27 (s, 2H), 3.07 (m, 2H), 1.84-1.68 (m, 6H), 1.65-1.50 (m, 2H), 1.17 (s, 6H), 1.06 (t, J=7.2 Hz, 3H). Mass (m/z): 291.3 [M+H]⁺. 320B (34.1 mg, 39%): ¹H NMR (400 MHZ, DMSO-d₆) δ 8.34 (s, 2H), 7.05 (d, J=8.4 Hz, 2H), 6.52 (d, J=8.4 Hz, 2H), 5.29 (s, 1H), 3.34 (m, 2H), 3.27 (s, 2H), 3.07 (m, 1H), 2.93 (m, 1H), 2.10-1.88 (m, 4H), 1.55-1.39 (m, 2H), 1.21 (m, 2H), 1.16 (s, 6H), 1.05 (t, J=7.2 Hz, 3H). Mass (m/z): 291.3 [M+H]⁺.

### Compound 321

### 4-((4-((4-(tert-butyl)phenyl)amino)cyclohexyl)amino) butan-1-ol

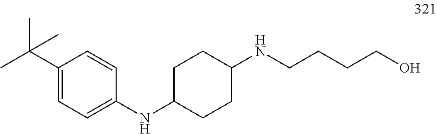 [SMILES: CC(C)(C)c1ccc(NC2CCC(NCCCCO)CC2)cc1]

The title compound 321 (60.9 mg) was prepared in a yield of 93.8% as a white powder with 1:1 mixture by ¹H NMR from 4-((4-(tert-butyl)phenyl)amino)cyclohexan-1-one (50 mg, 0.20 mmol), methyl 4-aminobutan-1-ol (54 mg, 0.61 mmol) and NaBH (OAc) 3 (86 mg, 0.41 mmol) according to the procedure for 20. ¹H NMR (400 MHZ, DMSO-d₆) δ 8.81 (s, 1H), 8.67 (s, 1H), 7.08 (dd, J=8.5, 5.9 Hz, 2H), 6.52 (dd, J=15.8, 8.5 Hz, 2H), 5.21 (s, 1H), 4.60 (q, J=5.2, 3.9 Hz, 1H), 3.42 (q, J=5.8 Hz, 2H), 2.98 (s, 2H), 2.90 (s, 2H), 2.15-1.97 (m, 3H), 1.87-1.73 (m, 2H), 1.68 (dq, J=15.5, 7.9, 7.5 Hz, 2H), 1.56 (t, J=13.6 Hz, 1H), 1.51-1.39 (m, 3H), 1.21 (d, J=1.5 Hz, 11H). Mass (m/z): 319.2 [M+H]⁺.

### Compound 322

### 2-(((4-((4-(tert-butyl)phenyl)amino)cyclohexyl)methyl)amino)ethan-1-ol

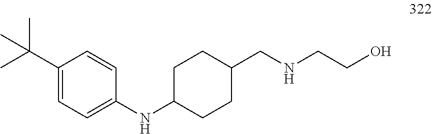 [SMILES: CC(C)(C)c1ccc(NC2CCC(CNCCO)CC2)cc1]

The title compound 322 (18 mg) was prepared in a yield of 30.79% as a white powder with 1:1 mixture by ¹H NMR from N-(4-(aminomethyl)cyclohexyl)-4-(tert-butyl)aniline (50 mg, 0.19 mmol), 2-bromoethan-1-ol (24 mg, 0.19 mmol) and potassium carbonate (40 mg, 0.29 mmol) according to the procedure for 12. ¹H NMR (400 MHZ, DMSO-d₆) δ 8.73 (s, 1H), 8.00 (s, 2H), 7.41 (s, 3H), 3.68 (dt, J=11.4, 5.4 Hz, 1H), 3.45 (s, 1H), 3.27 (s, 1H), 2.91 (s, 1H), 2.77 (s, 1H), 2.63 (t, J=6.3 Hz, 1H), 1.97 (d, J=11.9 Hz, 1H), 1.83 (d, J=14.4 Hz, 1H), 1.77-1.45 (m, 6H), 1.26 (d, J=3.6 Hz, 9H), 0.98 (d, J=12.5 Hz, 1H). Mass (m/z): 305.2 [M+H]⁺.

### Compound 323

### 2-(((1r,4r)-4-(((4-(tert-butyl)phenyl)amino)methyl)cyclohexyl)amino)ethan-1-ol

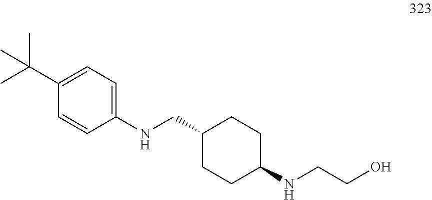 [SMILES: CC(C)(C)c1ccc(NC[C@H]2CC[C@H](NCCO)CC2)cc1]

The title compound 323 (25.9 mg) was prepared in a yield of 44.31% as a white powder from N-(((1r,4r)-4-aminocyclohexyl)methyl)-4-(tert-butyl)aniline (50 mg, 0.19 mmol), 2-bromoethan-1-ol (24 mg, 0.19 mmol) and potassium carbonate (40 mg, 0.29 mmol) according to the procedure for 12. ¹H NMR (400 MHZ, DMSO-d₆) δ 8.74 (s, 1H), 8.01 (s, 1H), 7.08 (d, J=8.2 Hz, 2H), 6.49 (s, 2H), 5.52-5.15 (m, 2H), 3.67 (s, 1H), 2.98 (s, 2H), 2.82 (d, J=6.4 Hz, 2H), 2.09 (d, J=12.0 Hz, 1H), 2.01-1.83 (m, 3H), 1.47 (s, 1H), 1.40-1.22 (m, 4H), 1.20 (s, 9H), 1.00 (q, J=12.4 Hz, 2H). Mass (m/z): 305.2 [M+H]⁺.

### Compound 324

### N-(2,2-dimethyl-2,3-dihydro-1H-inden-5-yl)cyclohexane-1,4-diamine

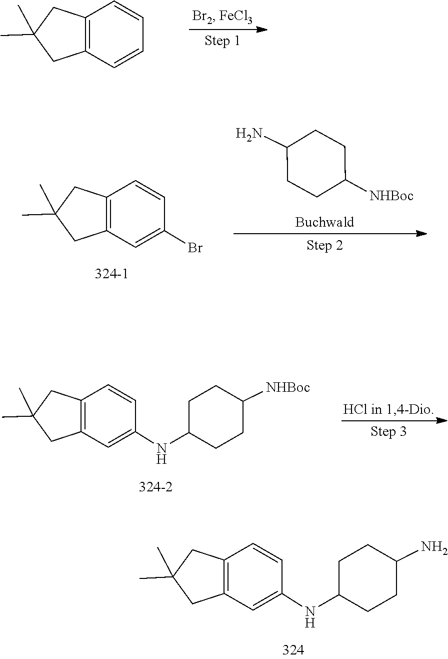 [SMILES: CC1(C)Cc2ccccc2C1 ; CC1(C)Cc2ccc(Br)cc2C1 ; CC(C)(C)OC(=O)NC1CCC(N)CC1 ; CC1(C)Cc2ccc(NC3CCC(NC(=O)OC(C)(C)C)CC3)cc2C1 ; CC1(C)Cc2ccc(NC3CCC(N)CC3)cc2C1]

Step 1. Preparation of 5-bromo-2,2-dimethyl-2,3-dihydro-1H-indene (324-1)

A solution of 2,2-dimethyl-2,3-dihydro-1H-indene (500 mg, 2.87 mmol), FeCl₃ (3.4 mg, 0.021 mmol) and Br₂ (460 mg, 2.87 mmol) in DCM (10 mL) was stirred at 0° C. for 18 hours. The mixture was concentrated under vacuum to afforded 300 mg (crude) of 324-1 (yield: 46.7%) as yellow oil.

Step 2. Preparation of tert-butyl (4-((2,2-dimethyl-2,3-dihydro-1H-inden-5-yl)amino)cyclohexyl)carbamate (324-2)

To a solution of 324-1 (300 mg, 1.33 mmol), tert-butyl (4-aminocyclohexyl)carbamate (314 mg, 1.33 mmol), Cs₂CO₃ (868 mg, 2.66 mmol) and Ruphos (124 mg, 0.26 mmol) in 1,4-dioxane (15 mL) was added Pd₂ (dba); (122 mg, 0.13 mmol). The mixture was stirred with refluxing overnight under N₂ atmosphere. After cooling to ambient temperature, ethyl acetate was added to the reaction mixture and the mixture was filtered through Celite. The filtrate was washed with water and saturated brine. The organic layer was dried over magnesium sulfate and concentrated under reduced pressure. The residue was purified by silica gel column to provide 100 mg of 324-2 (yield: 20.7%) as a yellow solid. Mass (m/z): 364.3 [M+H]⁺.

Step 3. Preparation of N-(2,2-dimethyl-2,3-dihydro-1H-inden-5-yl)cyclohexane-1,4-diamine (324)

324-2 (100 mg, 0.28 mmol) and HCl in 1,4-dioxane (10 mL, 4 N) were placed in a flask and stirred at 25° C. for 18 hrs. Excess 1,4-Dio. was removed under vacuum and the residue was purified by prep-HPLC (column-Xbridge-C18 150×19 mm, 5 μm; Mobile phase: ACN-H₂O (0.1% FA), 25%-40%) to afford 324 (11.4 mg) as a white solid. ¹H NMR (400 MHZ, DMSO-d₆) δ 6.83 (d, J=8.0 Hz, 1H), 6.38 (s, 1H), 6.31 (d, J=8.0 Hz, 1H), 5.06 (s, 1H), 3.06 (s, 1H), 2.87 (s, 1H), 2.53 (s, 4H), 1.95 (dd, J=22.8, 12.8 Hz, 4H), 1.36 (dd, J=22.0, 12.2 Hz, 2H), 1.22-1.09 (m, 2H), 1.07 (s, 6H). Mass (m/z): 259.3 [M+H]⁺.

### Compound 325

### N1-(1,1,3,3-tetramethyl-2,3-dihydro-1H-inden-5-yl)cyclohexane-1,4-diamine

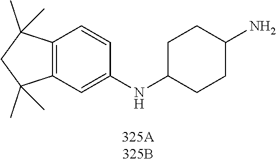 [SMILES: CC1(C)CC(C)(C)c2cc(NC3CCC(N)CC3)ccc21]

The title compounds 325A (45.7 mg) as a white solid and 325B (54.8 mg) as a white solid were prepared from tert-butyl (4-((1,1,3,3-tetramethyl-2,3-dihydro-1H-inden-5-yl)amino) cyclohexyl)carbamate (400 mg, 0.25 mmol), 1,4-dioxane (5 mL) and HCl/1,4-dioxane (10 mL) according to the procedure for 324. 325A: ¹NMR (400 MHZ, CD₃OD) δ 6.85 (d, J=8.0 Hz, 1H), 6.50 (dd, J=8.2, 2.2 Hz, 1H), 6.40 (d, J=2.2 Hz, 1H), 3.53 (s, 1H), 3.24-3.14 (m, 1H), 1.88-1.66 (m, 10H), 1.22 (d, J=7.4 Hz, 12H). Mass (m/z): 452.3 [M+H]⁺. HPLC: Rt=4.180 min (Column: XBRIDGE 3.5 μm, 2.1*50 mm, Mobile phase: H₂O (0.05% TFA)-ACN (0.05% TFA), ACN from 0 to 60% over 7 minutes, 7-8 min, ACN from 60% to 100%, Flow rate: 0.8 mL/min). 325B: ¹H NMR (400 MHZ, CD₃OD) δ 6.85 (d, J=8.0 Hz, 1H), 6.50 (dd, J=8.2, 2.2 Hz, 1H), 6.40 (d, J=2.2 Hz, 1H), 3.53 (s, 1H), 3.24-3.14 (m, 1H), 1.88-1.66 (m, 10H), 1.22 (d, J=7.4 Hz, 12H). Mass (m/z): 452.3 [M+H]⁺. HPLC: Rt=4.480 min (Column: XBRIDGE 3.5 μm, 2.1*50 mm, Mobile phase: H₂O (0.05% TFA)-ACN (0.05% TFA), ACN from 0 to 60% over 7 minutes, 7-8 min, ACN from 60% to 100%, Flow rate: 0.8 mL/min).

### Compound 326

### N-(4-(5-aminopentyl)cyclohexyl)-4-(tert-butyl)aniline

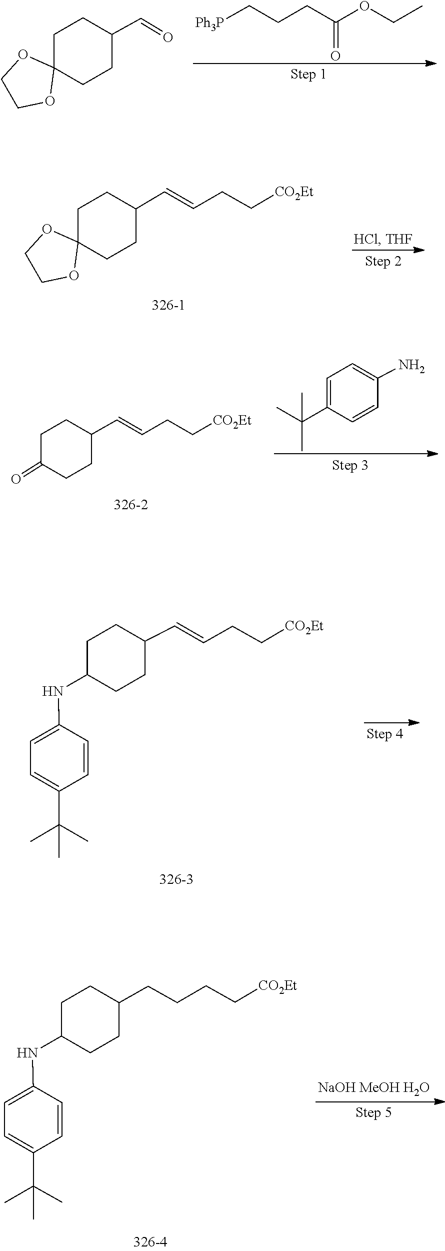

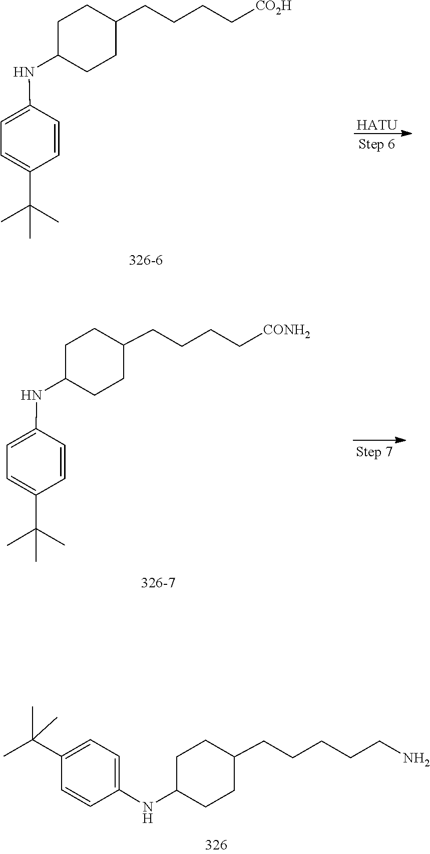 [SMILES: CC(C)(C)c1ccc(NC2CCC(CCCCC(=O)O)CC2)cc1 ; "HATU" ; CC(C)(C)c1ccc(NC2CCC(CCCCC(N)=O)CC2)cc1 ; CC(C)(C)c1ccc(NC2CCC(CCCCCN)CC2)cc1]

Step 1. Preparation of ethyl (E)-5-(1,4-dioxaspiro[4.5]decan-8-yl)pent-4-enoate (326-1) A solution of 1,4-dioxaspiro[4.5]decane-8-carbaldehyde (1000 mg, 5.9 mmol), NaH (940 mg, 23.6 mmol) and (4-ethoxy-4-oxobutyl) triphenylphosphonium bromide (5380 mg, 11.8 mmol) in THF (10 mL) was stirred at 0° C. under N₂ atmosphere for 18 hours. The solid was filtered and solvent was removed under vacuum. The residue was purified by silica gel column to provide 1 g of 326-1 (yield: 63.4%) as a yellow solid.

Step 2. Preparation of ethyl (E)-5-(4-oxocyclohexyl) pent-4-enoate (326-2)

326-1 (370 mg, 1.4 mmol) and HCl in THF (20 ml., 2 N) were placed in a flask and stirred at 25° C. for 18 hrs. Excess THF was removed under vacuum and the residue was purified by silica gel column to provide 320 mg of 326-2 (yield: 86.4%) as yellow oil.

Step 3. Preparation of ethyl (E)-5-(4-((4-(tert-butyl)phenyl)amino)cyclohexyl) pent-4-enoate (326-3)

A solution of 326-2 (320 mg, 1.42 mmol), 4-(tert-butyl)aniline (214 mg, 1.42 mmol) and NaBH₃CN (179 mg, 2.84 mmol) in MeOH (10 mL) was stirred at R.T. for 18 hours. The solid was filtered and solvent was removed under vacuum. The residue was purified by silica gel column to provide 225 mg of 326-3 (yield: 44.3%) as a yellow solid. Mass (m/z): 358.2 [M+H]

Step 4. Preparation of ethyl 5-(4-((4-(tert-butyl)phenyl)amino)cyclohexyl) pentanoate (326-4)

A solution of 326-3 (225 mg, 0.63 mmol), Pd/C (20 mg) in MeOH (10 mL) was stirred at 25° C. under H₂ atmosphere for 18 hours. The solid was filtered and solvent was removed under vacuum. The residue was purified by silica gel column to provide 120 mg of 326-4 (yield: 52.9%) as a yellow solid. Mass (m/z): 360.3 [M−H].

Step 5. Preparation of 5-(4-((4-(tert-butyl)phenyl)amino)cyclohexyl) pentanoic acid (326-5)

To a solution of 326-4 (120 mg, 0.33 mmol) in H₂O (5 mL) and THF (5 ml) was added NaOH (66 mg, 1.65 mmol). The mixture was stirred at 25° C. overnight. Ethyl acetate was added to the reaction mixture and the mixture was filtered through celite. The filtrate was washed with water and saturated brine. The organic layer was dried over magnesium sulfate and concentrated under reduced pressure. The residue was purified by silica gel column to provide 60 mg of 326-5 (yield: 56.3%) as a yellow oil. Mass (m/z): 332.3 [M+1]⁺.

Step 6. Preparation of 5-(4-((4-(tert-butyl)phenyl)amino)cyclohexyl) pentanamide (326-6)

A solution of 326-5 (87 mg, 0.26 mmol), NH₄Cl (56 mg, 1 mmol), HATU (149 mg, 0.39 mmol) and DIEA (169 mg, 1.3 mmol) in DMF (10 mL) was stirred at R.T. for 18 hours. The solid was filtered and solvent was removed under vacuum. The residue was purified by silica gel column to provide 40 mg of 326-6 (yield: 46.4%) as a yellow solid. Mass (m/z): 331.3 [M+H]⁺.

Step 7. Preparation of N-(4-(5-aminopentyl)cyclohexyl)-4-(tert-butyl)aniline (326)

A solution of 326-6 (40 mg, 0.12 mmol), BH₃-THF (10 mL) in THF (5 mL) was stirred at 70° C. for 18 hours. The solid was filtered and solvent was removed under vacuum. The residue was purified by prep-HPLC (column-Xbridge-C18 150×19 mm, 5 μm; Mobile phase: ACN-H₂O (0.1% FA), 25%-40%) to afford 326 (5.3 mg) as a white solid. ¹H NMR (400 MHZ, DMSO-d₆) δ 7.05 (d, J=8.8 Hz, 2H), 6.46 (d, J=8.8 Hz, 2H), 5.09 (s, 1H), 3.05 (s, 2H), 2.70 (t, J-7.2 Hz, 2H), 1.96 (d, J=12.0 Hz, 2H), 1.73 (d, J=12.0 Hz, 2H), 1.49 (s, 2H), 1.25 (d, J=15.6 Hz, 6H), 1.16 (d, J=30.8 Hz, 9H), 1.01 (dd, J=24.8, 11.9 Hz, 5H). Mass (m/z): 317.3 [M+H]⁺.

### Compound 327

### 2-amino-5-((4-(tert-butyl)phenyl)amino)cyclohexan-1-ol

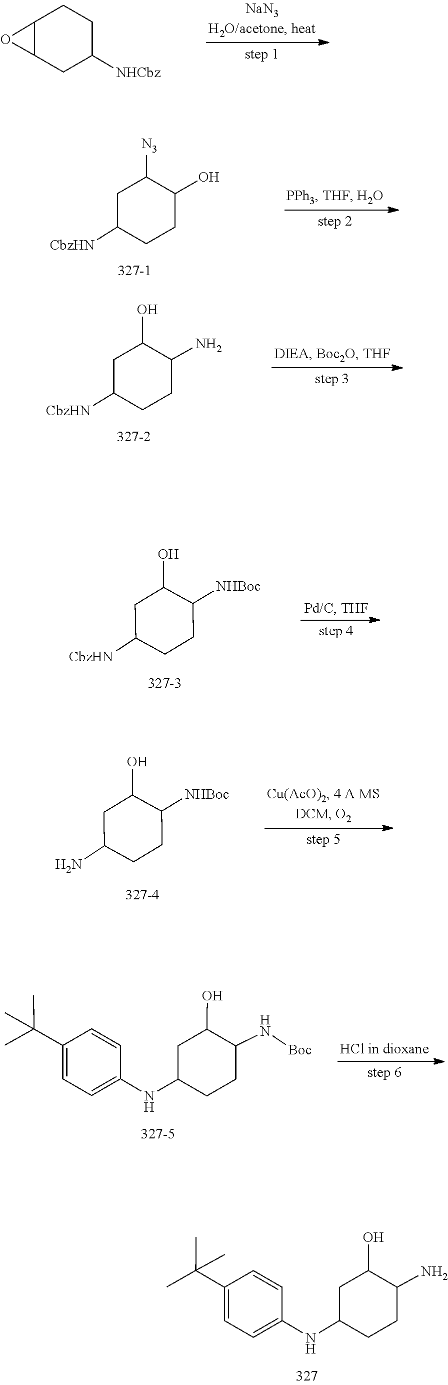

Step 1. Preparation of benzyl (3-azido-4-hydroxycyclohexyl)carbamate (327-1)

To a solution of benzyl (7-oxabicyclo[4.1.0]heptan-3-yl)carbamate (0.9 g, 3.6 mmol) in Acetone (10 mL) and H₂O (1 mL) was added NaN₃ (0.26 g, 4 mmol) at 25° C. Then the mixture was stirred at 70° C. for 4 hrs. LCMS showed the reaction was completed. The reaction was quenched with water (20 mL), extracted with EA (20 mL*3). The combined organic layers were washed with brine (40 mL), dried over sodium sulfate, filtered and concentrated to give 327-1 (0.7 g, 66.3% yield) as yellow oil. MS (m/z): 313.1 [M+Na]⁺.

Step 2. Preparation of benzyl (4-amino-3-hydroxycyclohexyl)carbamate (327-2)

To a solution of 327-1 (0.7 g, 2.4 mmol) in THF (10 mL) and H₂O (2 mL) was added PPh₃ (500 mg, 1.90 mmol) under N₂ at 25° C. Then the mixture was stirred at 25° C. overnight. LCMS showed the reaction was completed. The mixture was added into H₂O and wash with EA. The water phase was concentrated to give 327-2 (700 mg, 100% yield) as a yellow solid. MS (m/z): 265.2 [M+H]⁺.

Step 3. Preparation of benzyltert-butyl (2-hydroxycyclohexane-1,4-diyl) dicarbamate (327-3)

To a solution of 327-2 (0.7 g, 2.6 mmol) in THF (10 mL) was added BoczO (630 mg, 2.9 mmol) and DIEA (513 mg, 4 mmol) at 25° C. Then the mixture was stirred at 25° C. for 4 h. LCMS showed the reaction was completed. The reaction was quenched by water (20 mL) slowly, extracted with EA (20 mL*3). The combined organic layers were washed with brine (40 mL), dried over sodium sulfate, filtered and concentrated. The residue was purified by combi-flash with EA/PE (1:2) to afford 327-3 (0.5 g, 60% yield) as a white solid. MS (m/z): 387.2 [M+Na]⁺.

Step 4. Preparation of tert-butyl (4-amino-2-hydroxycyclohexyl)carbamate (327-4)

To a solution of 327-3 (550 mg, 1.51 mmol) in THF (10 mL) was added wet Pd/C (80 mg) and the mixture was stirred at 25° C. for 8 hrs. LCMS showed the reaction was completed. The reaction was filtered and concentrated to give 327-4 (120 mg, 34.6% yield) as a yellow solid. MS (m/z): 230.2 [M+H]⁺.

Step 5. Preparation of tert-butyl (4-((4-(tert-butyl)phenyl)amino)-2-hydroxycyclo hexyl)carbamate (327-5)

To a solution of 327-4 (0.12 g, 0.52 mmol) in DCM (10 mL) was added (4-(tert-butyl)phenyl) boronic acid (92.8 mg, 0.52 mmol), Cu (AcO) 2 (10 mg, 0.052 mmol) and 4 A MS (100 mg) under O₂ at 25° C. Then the mixture was stirred at 25° C. overnight. LCMS showed the reaction was completed. The reaction was quenched by water (20 mL) slowly, extracted with EA (20 mL*3). The combined organic layers were washed with brine (40 mL), dried over sodium sulfate, filtered and concentrated. The residue was purified by combi-flash with EA/PE (1:1) to afford 327-5 (0.11 g, 58.5% yield) as a yellow solid. MS (m/z): 362.9 [M+H]⁺.

Step 6. Preparation of 2-amino-5-((4-(tert-butyl)phenyl)amino)cyclohexan-1-ol (327)

To a solution of 327-5 (110 mg, 0.30 mmol) in DCM (4 mL) was added HCl in dioxane (4 mL), and the mixture was stirred at 25° C. for 2 hrs. LCMS showed the reaction was completed. The residue was concentrated. The residue was purified by Prep-HPLC to give 327 (15 mg, 18.8% yield) as a yellow solid. ¹H NMR (400 MHZ, CD₃OD) δ 7.60 (d, J=7.6 Hz, 2H), 7.43 (d, J=7.6 Hz, 2H), 3.97-3.85 (m, 1H), 3.75-3.65 (m, 1H), 3.52-3.42 (m, 1H), 2.42-2.25 (m, 1H), 2.09-1.75 (m, 5H), 1.32 (s, 9H). MS (m/z): 263.0 [M+H]⁺.

### Compound 328

### N-((3-aminocyclopentyl)methyl)-4-(tert-butyl)aniline

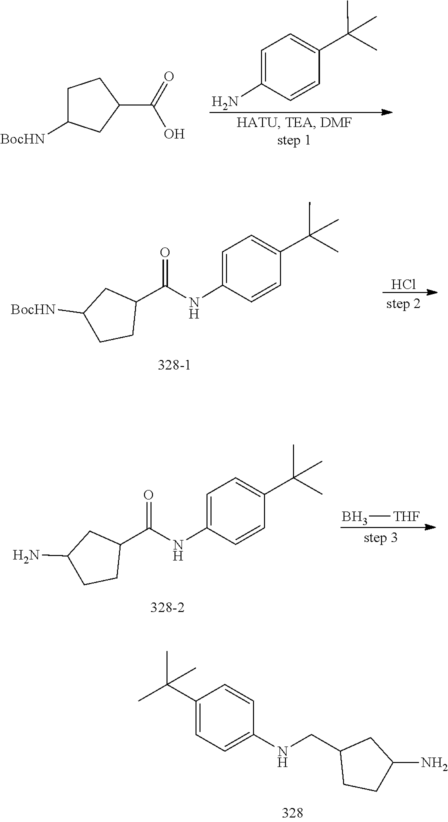

Step 1. Preparation of tert-butyl (3-((4-(tert-butyl)phenyl) carbamoyl)cyclopentyl)carbamate (328-1)

A mixture of 3-((tert-butoxycarbonyl)amino)cyclopentane-1-carboxylic acid (300 mg, 1.31 mmol), 4-(tert-butyl)aniline (234 mg, 1.57 mmol), HATU (597 mg, 1.57 mmol), DIPEA (254 mg, 1.96 mmol) in DMF (5 mL) was stirred overnight at room temperature. Then 40 mL of water was added. The solid was purified by silica gel chromatography. Target product (430 mg, 91%) was obtained as a yellow solid. Mass (m/z): 383.3 [M+H]⁺.

Step 2. Preparation of 3-amino-N-(4-(tert-butyl)phenyl)cyclopentane-1-carboxamide (328-2)

To a solution of tert-butyl (3-((4-(tert-butyl)phenyl) carbamoyl)cyclopentyl)carbamate (430 mg, 1.92 mmol) in HCl in dioxane (5 ml) and in CH₂Cl₂ (5 mL). Then the mixture was stirred for overnight at rt. The reaction was concentrated under vacuum. The residue was purified by prep-TLC to afford the desired product (305 mg, 97%) as a white solid. Mass (m/z): 261.3 [M+H]⁺.

Step 3. Preparation of N-((3-aminocyclopentyl)methyl)-4-(tert-butyl)aniline (328)

To a solution of 3-amino-N-(4-(tert-butyl)phenyl)cyclopentane-1-carboxamide (305 mg, 1.17 mmol) in BH₃-THF (20 mL). Then the mixture was stirred overnight at 70° C. The reaction was concentrated under vacuum. The residue was purified by prep-HPLC to afford the desired product (45.2 mg, 16%) as a white solid. ¹H NMR (400 MHZ, CD₃OD) δ 7.15 (d, J=8.8 Hz, 2H), 6.60 (d, J=8.7 Hz, 2H), 3.49-3 36 (m, 1H), 3.07 (d, J=6.6 Hz, 1H), 2 34-2.16 (m, 2H), 2.08-1.80 (m, 2H), 1.61-1.46 (m, 2H), 1.34-1.29 (m, 1H), 1.25 (s, 9H). Mass (m/z): 247.3 [M+H]⁺.

### Compound 329

### 1-N-(4-(tert-butyl)-3-ethylphenyl)cyclohexane-1,4-diamine

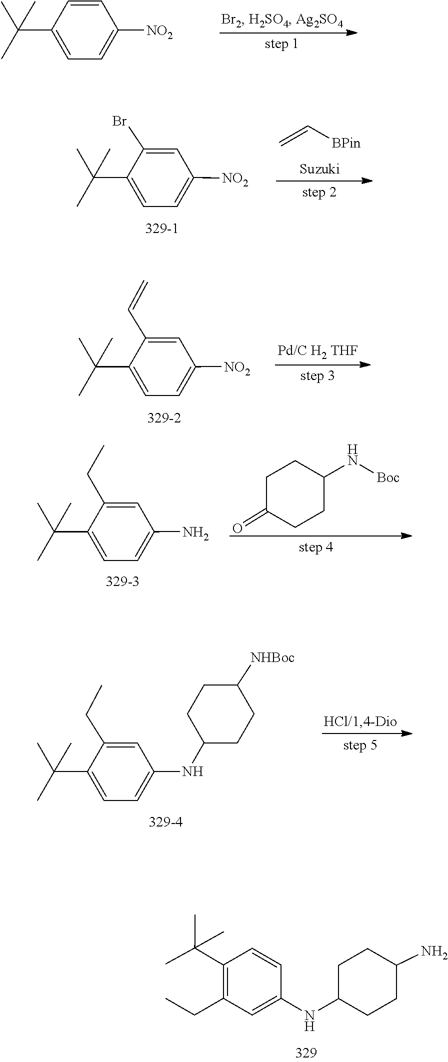

Step 1. Preparation of 2-bromo-1-(tert-butyl)-4-nitrobenzene (329-1)

A mixture of 1-(tert-butyl)-4-nitrobenzene (1 g, 5.6 mmol), Ag₂SO₄ (2.97 g, 9.5 mmol) in H₂SO₄ (10 mL) was added Br₂ (0.89 g, 5.6 mmol) and stirred at rt for 16 hours. Pour the mixture into a diluted NaHSO₃ solution and extract with ethyl acetate (3×100 mL). It was quenched by H₂O (300 mL) and extracted with EA (3×300 mL), dried with MgSO₄, filtered and concentrated to afford target product (600 mg, yield: 33.3%) as a black solid.

Step 2. Preparation of 1-(tert-butyl)-4-nitro-2-vinylbenzene (329-2)

To a solution of 329-1 (600 mg, 2.32 mmol) in 1,4-dioxane (10 mL) was added Pd(PPh₃)₄ mmol), mmol) (268.62 mg, 0.23 K₂CO₃ (962.38 mg, 6.97 and 4,4,5,5-tetramethyl-2-vinyl-1,3,2-dioxaborolane (999.75 mg, 5.814 mmol). Then the mixture was stirred overnight at 90° C. It was quenched by H₂O (100 mL) and extracted with EA (3×100 mL). The crude product was applied onto a silica gel column (12 g) eluted with PE: EA (10:1) to give product (300 mg, yield: 50.3%) as a yellow solid.

Step 3. Preparation of 4-(tert-butyl)-3-ethylaniline (329-3)

To a solution of 329-2 (300 mg, 1.46 mmol) in THF (10 mL) was added Pd/C (15.55 mg, 0.14 mmol). Then the mixture was stirred overnight at rt. It was quenched by H₂O (100 mL) and extracted with EA (3×100 mL). The organic layers were combined, washed with brine NaCl (2×100 mL) and dried with MgSO₄, filtered and concentrated to afford target product (150 mg, yield: 46.3%) was obtained as a yellow solid. Mass (m/z): 178.2 [M+H]⁺.

Step 4. Preparation of 4-cyclobutylaniline (329-4)

A mixture of 1-N-(4-(tert-butyl)phenyl)cyclohexane-1,4-diamine (150 mg, 0.84 mmol), tert-butyl (3-oxopropyl)carbamate (181.3 mg, 0.84 mmol), Pic-BH₃ (135.75 mg, 1.27 mmol) in AcOH/H₂O=1:10 (5.5 mL) was stirred overnight at 25° C. The pH of the water phase was adjusted to 8 by aq. NaHCO₃. It was quenched by H₂O (30 mL) and extracted with EA (3×30 mL), dried with MgSO₄, filtered and concentrated to afford the target product (200 mg, yield: 37.8%) as a yellow solid. Mass (m/z): 375.3 [M+H]⁺.

Step 5. Preparation of 1-N-(4-(tert-butyl)-3-ethylphenyl)cyclohexane-1,4-diamine (329)

To a solution of 329-4 (200 mg, 0.53 mmol) in 1,4-dioxane (5 mL) was added 4 N HCl in 1,4-dioxane (10 mL). Then the mixture was stirred overnight at 25° C. The reaction was concentrated under vacuum. The residue was purified by prep-TLC to afford the desired product 329 (22.8 mg) as a white solid. ¹H NMR (400 MHZ, CD₃OD) δ 7.07 (d, J=8.6 Hz, 1H), 6.50 (d, J=2.8 Hz, 1H), 6.38 (dd, J=8.6, 2.8 Hz, 1H), 3.52 (s, 1H), 3.25-3.12 (m, 1H), 2.77 (q, J=7.6 Hz, 2H), 1.91-1.70 (m, 8H), 1.32 (s, 9H), 1.20 (t, J=7.4 Hz, 3H). Mass (m/z): 275.0 [M+H]⁺.

### Compound 330

### N1-(1,1-dimethyl-2,3-dihydro-1H-inden-5-yl)cyclohexane-1,4-diamine

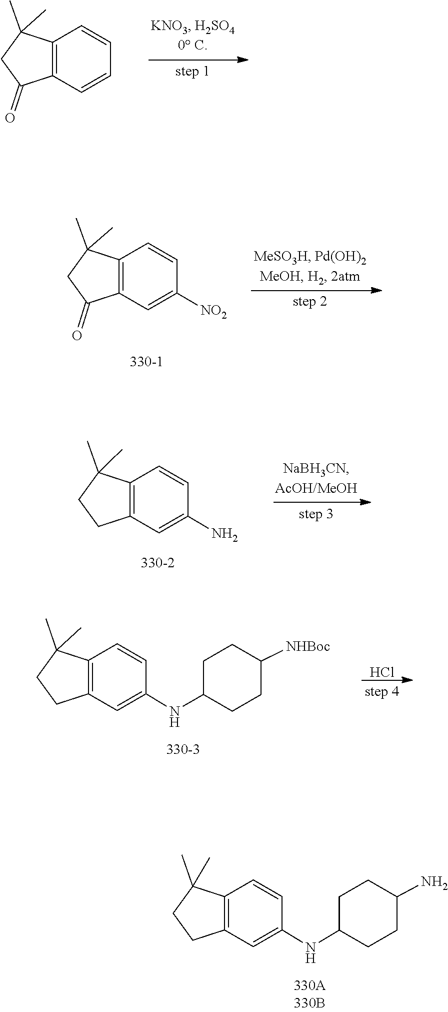 [SMILES: CC1(C)CC(=O)c2ccccc21 ; CC1(C)CC(=O)c2cc([N+](=O)[O-])ccc21 ; CC1(C)CCc2cc(N)ccc21 ; CC(C)(C)OC(=O)NC1CCC(Nc2ccc3c(c2)CCC3(C)C)CC1 ; Cl ; CC1(C)CCc2cc(NC3CCC(N)CC3)ccc21]

Step 1. Preparation of 3,3-dimethyl-6-nitro-2,3-dihydro-1H-inden-1-one (330-1)

To a solution of 3,3-dimethyl-2,3-dihydro-1H-inden-1-one (2 g, 12.5 mmol) in H₂SO₄ (10 mL) was added KNO₃ (1.39 g, 13.7 mmol) at 0° C. Then the mixture was stirred at 0° C. for 2 hrs. LCMS showed the reaction was completed. The reaction was quenched with ice-water (20 mL). The mixture was filtered to give 330-1 (1.6 g, 62.5% yield) as a yellow solid. MS (m/z): 206.1 [M+H]⁺.

Step 2. Preparation of 1,1-dimethyl-2,3-dihydro-1H-inden-5-amine (330-2)

To a solution of 3,3-dimethyl-6-nitro-2,3-dihydro-1H-inden-1-one (0.2 g, 0.97 mmol) in MeOH (10 mL) was added MeSO₃H (93 mg, 0.97 mmol) and Pd(OH)₂/C (50 mg) under H₂ at 25° C. Then the mixture was stirred at 25° C. and 2 atm overnight. LCMS showed the reaction was completed. The mixture was filtered and concentrated to give 330-2 (0.15 g, 95.5% yield) as a brown oil. MS (m/z) 162.2 [M+H]⁺.

Step 3. Preparation of tert-butyl (4-((1,1-dimethyl-2,3-dihydro-1H-inden-5-yl)amino)cyclohexyl)carbamate (330-3)

To a solution of 1,1-dimethyl-2,3-dihydro-1H-inden-5-amine (0.15 g, 0.93 mmol) in MeOH (10 mL) and AcOH (0.1 mL) was added tert-butyl (4-oxocyclohexyl)carbamate (218 mg, 1.02 mmol) at 25° C. Then the mixture was stirred at 50° C. for 2 h. Then the mixture was added NaBH₃CN (175 mg, 2.79 mmol) after cooling to 25° C. The mixture was stirred at 25° C. for 6 h. LCMS showed the reaction was completed. The reaction was quenched by ice water (20 mL) slowly, extracted with EA (20 mL*3). The combined organic layers were washed with brine (40 mL), dried over sodium sulfate, filtered and concentrated. The residue was purified by combi-flash with EA/PE (1:1) to afford 330-3 (0.14 g, 42% yield) as a yellow solid. MS (m/z) 359.2 [M+H]⁺.

Step 4. Preparation of N1-(1,1-dimethyl-2,3-dihydro-1H-inden-5-yl)cyclohexane-1,4-diamine (330)

To of tert-butyl a solution (4-((1, 1-dimethyl-2,3-dihydro-1H-inden-5-yl)amino)cyclohexyl)carbamate (140 mg. 0.39 mmol) in DCM (5 mL) was added HCl in dioxane (5 mL), and the mixture was stirred at 25° C. for 2 hrs. LCMS showed the reaction was completed. The residue was concentrated. The residue was purified by Prep-HPLC to give 330A (16 mg, 15.8% yield) as a white solid and 330B (21.2 mg, 21% yield) as a white solid. 330A: ¹H NMR (300 MHZ, CD₃OD) δ 7.30 (d, J=8.0 Hz, 1H), 7.27-7.16 (m, 2H), 3.55-3.40 (m, 1H), 3.20-3.05 (m, 1H), 2.92 (t, J=7.2 Hz, 2H), 2.20-2.05 (m, 4H), 1.95 (t, J=7.2 Hz, 2H), 1.65-1.37 (m, 4H), 1.23 (s, 6H). MS (m/z): 259.3 [M+H]⁺. HPLC: Rt: 3.807 min (Column: XBRIDGE 2.1*50 mm, 3.5 μm; Mobile Phase: H₂O (0.05% TFA)/ACN (0.05% TFA), ACN from 0% to 60% over 7 minutes, 7-8 min, ACN from 60% to 100%; 0.8 mL/min). 330B: ¹H NMR (300 MHZ, CD₃OD) δ 7.39-7.25 (m, 3H), 3.70-3.50 (m, 1H), 3.45-3.35 (m, 1H), 2.92 (t, J=7.2 Hz, 2H), 2.10-1.80 (m, 10H), 1.23 (s, 6H). MS (m/z): 259.3 [M+H]⁺. HPLC: Rt: 3.915 min (Column: XBRIDGE 2.1*50 mm, 3.5 μm; Mobile Phase: H₂O (0.05% TFA)/ACN (0.05% TFA), ACN from 0% to 60% over 7 minutes, 7-8 min, ACN from 60% to 100%; 0.8 mL/min).

### Compound 331

### N1-(3,5-dimethylphenyl)cyclohexane-1,4-diamine

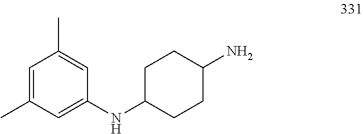 [SMILES: Cc1cc(C)cc(NC2CCC(N)CC2)c1]

The title compound 331 (81.1 mg) was prepared in a yield of 90.0% as a white powder from 3,5-dimethylaniline (50 mg, 0.41 mmol), tert-butyl (4-oxocyclohexyl)carbamate (132 mg, 0.61 mmol) according to the procedure for 20. ¹H NMR (400 MHZ, DMSO-d₆) δ 7.96 (d, J=22.4 Hz, 2H), 6.20 (s, 1H), 6.19-6.11 (m, 2H), 5.18 (s, 1H), 3.40 (s, 1H), 3.08 (d, J=11.5 Hz, 1H), 2.97 (s, 1H), 2.12 (d, J=2.0 Hz, 6H), 2.05-1.91 (m, 2H), 1.81-1.65 (m, 2H), 1.51-1.35 (m, 1H), 1.21-1.08 (m, 1H). Mass (m/z): 219.4 [M+H]⁺.

### Compound 332

### 3-((4-(tert-pentyl)phenyl)amino)cyclopentane-1-carboxamide

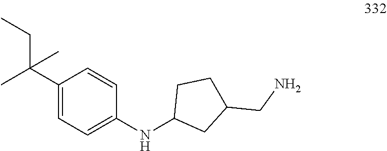 [SMILES: CCC(C)(C)c1ccc(NC2CCC(CN)C2)cc1]

The title compound 332 (15.6 mg) was prepared in a total yield of 20.0% as a white solid with 3:2 mixture by ¹H NMR from 3-((4-(tert-pentyl)phenyl)amino)cyclopentane-1-carboxamide (82 mg, 0.3 mmol) and LiAlH₄ (34 mg, 0.9 mmol) according to the procedure for 118. ¹H NMR (400 MHZ, Methanol-d₄) δ 7.10-7.03 (m, 2H), 6.62-6.56 (m, 2H), 3.88-3.76 (m, 1H), 2.76 (dd, J=12.7, 7.3 Hz, 2H), 2.34-1.75 (m, 4H), 1.65-1.33 (m, 4H), 1.19 (s, 6H), 0.63 (t, J=7.4 Hz, 3H). Mass (m/z): 276.3 [M+H]⁺.

### Compound 333

### N-(3-(aminomethyl)cyclobutyl)-4-(tert-pentyl)aniline

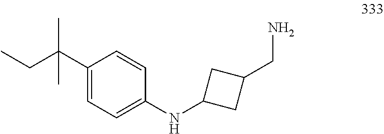 [SMILES: CCC(C)(C)c1ccc(NC2CC(CN)C2)cc1]

The title compound 333 (30.5 mg) was prepared in a total yield of 38.8% as a white solid with 3:2 mixture by ¹H NMR from 3-((4-(tert-pentyl)phenyl)amino)cyclobutane-1-carboxamide (83 mg, 0.32 mmol) and LiAlH₄ (36 mg, 0.96 mmol) according to the procedure for 118. ¹H NMR (301 MHz, Methanol-d₄) δ 7.07-7.00 (m, 2H), 6.46-6.58 (m, 2H), 4.00-3.86 (m, 0.4H), 3.84-3.68 (m, 0.6H), 95 (d, J=7.2 Hz, 0.8H), 2.84 (d, J=7.2 Hz, 1.2H), 2.60-2.47 (m, 2H), 2.28-2.13 (m, 1.2 H), 2.09-1.97 (m, 0.8H), 1.60-1.47 (m, 2H), 0.59 (t, J=7.4 Hz, 3H). Mass (m/z): 247.3 [M+H]⁺.

### Compound 334

### 4-(tert-butyl)-N-(4-methylcyclohexyl)aniline

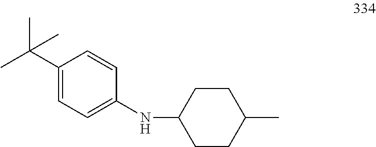 [SMILES: CC1CCC(Nc2ccc(C(C)(C)C)cc2)CC1]

The title compound 334 (565 mg, 86.1%) as a yellow oil was prepared from 4-(tert-butyl)aniline (400 mg, 2.68 mmol), 4-methylcyclohexan-1-one (361 mg, 3.22 mmol), DCM (5 mL), and sodium triacetoxyborohydride (1.14 g, 5.36 mmol) according to the procedure for 1-1. ¹H NMR (400 MHz, DMSO-d₆) δ 7.10-7.00 (m, 2H), 6.54-6.44 (m, 2H), 5.12 (dd, J=35.9, 8.0 Hz, 1H), 3.37 (s, 1H), 1.93 (d, J=11.6 Hz, 1H), 1.69-1.28 (m, 7H), 1.20 (d, J=1.3 Hz, 9H), 1.05 (t, J=9.2 Hz, 1H), 0.89 (dd, J=8.2, 6.5 Hz, 3H). Mass (m/z): 246.2 [M+H]⁺

### Compound 335

### N-((3-aminocyclobutyl)methyl)-4-(tert-butyl)aniline

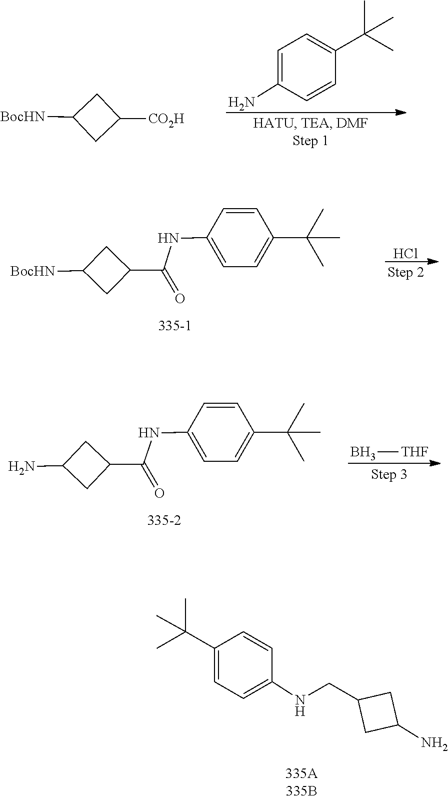

Step 1. Preparation of tert-butyl (3-((4-(tert-butyl)phenyl) carbamoyl)cyclobutyl)carbamate (335-1)

A mixture of 3-((tert-butoxycarbonyl)amino)cyclopentane-1-carboxylic acid (300 mg, 1.31 mmol), 4-(tert-butyl)aniline (250 mg, 1.67 mmol), HATU (636 mg, 1.67 mmol), DIPEA (270 mg, 2.09 mmol) in DMF (5 mL) was stirred overnight at room temperature. Then 40 mL of water was added. The solid was purified by silica gel chromatography. The target product (451 mg, 93%) was obtained as a yellow solid. Mass (m/z): 346.9 [M+H]⁺.

Step 2. Preparation of 3-amino-N-(4-(tert-butyl)phenyl)cyclobutane-1-carboxamide (335-2)

To a solution of tert-butyl (3-((4-(tert-butyl)phenyl) carbamoyl)cyclobutyl)carbamate (451 mg, 1.30 mmol) in CH₂Cl₂ (5 mL) was added HCl in dioxane (4N; 5 mL). Then the mixture was stirred for overnight at rt. The reaction was concentrated under vacuum. The residue was purified by prep-TLC to afford the desired product (303 mg, 92%) as a white solid. Mass (m/z): 247.2 [M+H]⁺.

Step 3. Preparation of N-((3-aminocyclobutyl)methyl)-4-(tert-butyl)aniline (335)

To a solution of 3-amino-N-(4-(tert-butyl)phenyl)cyclobutane-1-carboxamide (303 mg, 1.23 mmol) in BH₃-THF (20 mL). Then the mixture was stirred overnight at 70° C. The reaction was concentrated under vacuum. The residue was purified by prep-HPLC to afford the desired product 335A (27.2 mg, 7%) as a white solid and 335B (9.8 mg, 4%) as a white solid. 335A:

¹H NMR (400 MHZ, CD₃OD) δ 7.15 (d, J=8.7 Hz, 2H), 6.59 (d, J=8.7 Hz, 2H), 3.43-3.37 (m, 1H), 3.09 (d, J=6.5 Hz, 2H), 2.47-2.40 (m, 2H), 2.30-2.21 (m, 1H), 1.69-1.60 (m, 2H), 1.25 (s, 9H). Mass (m/z): 233.3 [M+H]⁺. HPLC: Rt: 3.621 min (Column: XBRIDGE 2.1*50 mm, 3.5 μm; Mobile Phase: H₂O (0.05% TFA)/ACN (0.05% TFA), ACN from 0% to 60% over 7 minutes, 7-8 min, ACN from 60% to 100%; 0.8 mL/min). 335B: ¹H NMR (400 MHz, CD₃OD) δ 7.15 (d, J=8.7 Hz, 2H), 6.60 (d, J=8.7 Hz, 2H), 3.81-3.66 (m, 1H), 3.17 (d, J=7.6 Hz, 2H), 2.67-2.60 (m, 1H), 2.27-2.03 (m, 4H), 1.25 (s, 9H). Mass (m/z): 233.3 [M+H]⁺. HPLC: Rt: 3.714 min (Column: XBRIDGE 2.1*50 mm, 3.5 μm; Mobile Phase: H₂O (0.05% TFA)/ACN (0.05% TFA), ACN from 0% to 60% over 7 minutes, 7-8 min, ACN from 60% to 100%; 0.8 mL/min).

### Compound 336

### N-(2-(4-aminocyclohexyl)ethyl)-4-(tert-butyl)aniline

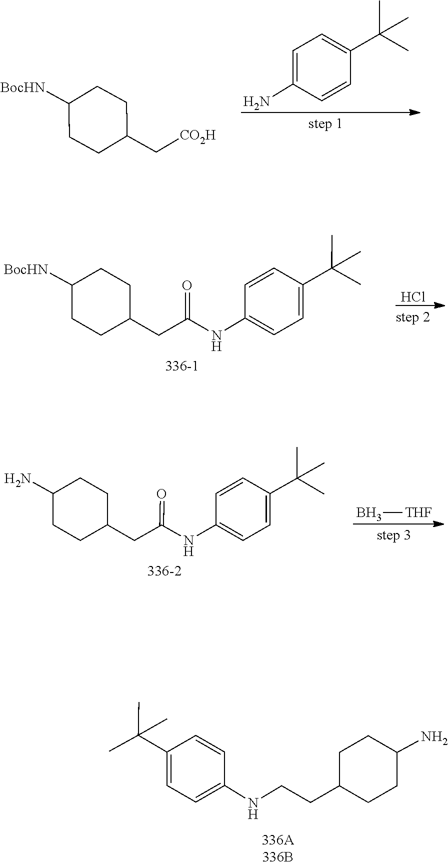

Step 1. Preparation of tert-butyl (4-(2-((4-(tert-butyl)phenyl)amino)-2-oxoethyl)cyclohexyl)carbamate (336-1)

To a solution of 2-(4-((tert-butoxycarbonyl)amino)cyclohexyl) acetic acid (0.2 g, 0.78 mmol) in DMF (10 mL) was added 4-(tert-butyl)aniline (0.14 g, 0.94 mmol), HATU (0.44 mg, 1.17 mmol), DIEA (0.3 g, 2.34 mmol) the mixture was stirred at 25° C. for 2 h. Diluting with water (30 mL), the reaction was extracted by EA (20 mL) for 3 times. The organic phase was washed 3 times by NaCl aq. (20 mL), dried over sodium sulfate, concentrated under vacuum and the residue was purified by flash chromatography to afford the desired product (0.15 g, 49.5%) as colorless oil. Mass (m/z): 333.2 [M+H]⁺.

Step 2. Preparation of 2-(4-aminocyclohexyl)-N-(4-(tert-butyl)phenyl) acetamide (336-2)

To a solution of tert-butyl (4-(2-((4-(tert-butyl)phenyl)amino)-2-oxoethyl)cyclohexyl)carbamate (0.15 g, 0.39 mmol) in THF (5 mL) was added HCl in dioxane (5 mL) and the mixture was stirred for 2 h. Quenched with NaHCO₃ (10 mL), the reaction was extracted by EA (10 mL) for 3 times. The combined organic layers were dried over sodium sulfate, concentrated under vacuum and the residue was purified by flash chromatography to afford the desired product (0.1 g, 88.9%) as colorless oil. Mass (m/z): 289.2 [M+H]⁺.

Step 3. Preparation of N-(2-(4-aminocyclohexyl)ethyl)-4-(tert-butyl)aniline (336)

To a solution of 2-(4-aminocyclohexyl)-N-(4-(tert-butyl)phenyl) acetamide (0.2 g, 0.5 mmol) in THF (2 mL) was added BH₃-THF (15 mL) and the mixture was stirred at 70° C. for 2 h. Quenched with water (10 mL), the reaction was extracted by EA (10 mL) for 3 times. The combined organic layers were dried over sodium sulfate, concentrated under vacuum and the residue was purified by prep-HPLC (column-Xbridge-C18 150×21.2 mm, 5 μm; Mobile phase: ACN-H₂O (0.1% FA), 40%-60%) to afford the desired product 336A (11.1 mg) as a black solid and 336B (3.4 mg) as a black solid. 336A: ¹H NMR (400 MHZ, CDCl₃) δ 7.19 (d, J=8.4, 2H), 6.54 (d, J=8.4, 2H), 3.09 (t, J=6.8, 2H), 2.98 (s, 1H), 2.08 (s, 2H), 1.86 (d, J=11.6, 2H), 1.51-1.42 (m, 5H), 1.27 (s, 9H), 1.01 (d, J=12.0, 2H). Mass (m/z): 274.9 [M+H]⁺. HPLC: Rt=3.801 min (Column: XBRIDGE 3.5 μm, 2.1*50 mm, Mobile phase: H₂O (0.05% TFA)-ACN (0.05% TFA), ACN from 0 to 60% over 7 minutes, 7-8 min, ACN from 60% to 100%, Flow rate: 0.8 mL/min). 336B: ¹H NMR (400 MHZ, CDCl₃) δ 7.23 (d, J=8.4, 2H), 6.72 (d, J=8.4, 2H), 3.42 (s, 1H), 3.12 (s, 2H), 1.92 (s, 2H), 1.72 (s, 4H), 1.60 (s, 5H), 1.27 (s, 9H). Mass (m/z): 274.9 [M+H]⁺. HPLC: Rt=4.110 min (Column: XBRIDGE 3.5 μm, 2.1*50 mm, Mobile phase: H₂O (0.05% TFA)-ACN (0.05% TFA), ACN from 0 to 60% over 7 minutes, 7-8 min, ACN from 60% to 100%, Flow rate: 0.8 mL/min).

### Compound 337

### 1-N-(5,5-dimethyl-5,6,7,8-tetrahydronaphthalen-2-yl)cyclohexane-1,4-diamine

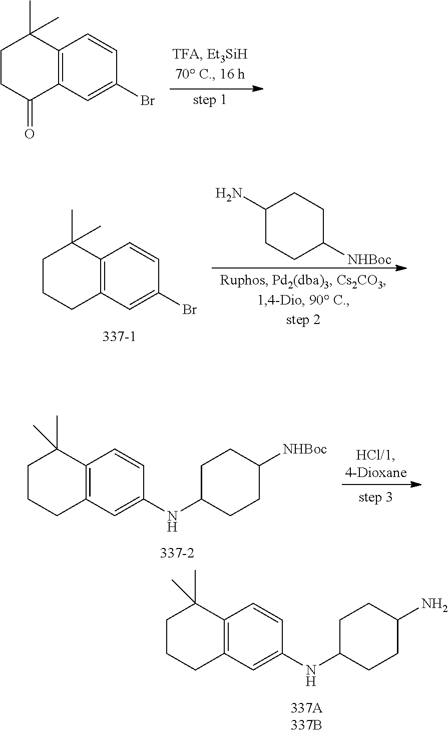 [SMILES: CC1(C)CCC(=O)c2cc(Br)ccc21 ; CC1(C)CCCc2cc(Br)ccc21 ; CC(C)(C)OC(=O)NC1CCC(N)CC1 ; CC(C)(C)OC(=O)NC1CCC(Nc2ccc3c(c2)CCCC3(C)C)CC1 ; CC1(C)CCCc2cc(NC3CCC(N)CC3)ccc21]

Step 1. 6-bromo-1,1-dimethyl-1,2,3,4-tetrahydronaphthalene (337-1)

A mixture of 7-bromo-4,4-dimethyl-3,4-dihydronaphthalen-1 (2H)-one (500 mg, 1.97 mmol) in TFA (7 mL) was added Et₃SiH (2.3 g, 19.8 mmol). It was quenched by H₂O (30 mL) and extracted with DCM (3×30 mL). The organic layers were combined, washed with brine NaCl (2×30 mL) and dried with MgSO₄, filtered and concentrated to afford the target product (500 mg, yield: 95.2%) as a yellow oil.

Step 2. tert-butyl (4-((5,5-dimethyl-5,6,7,8-tetrahydronaphthalen-2-yl)amino)cyclo-hexyl)carbamate (337-2)

To a solution of 337-1 (300 mg, 1.25 mmol) in 1,4-dioxane (15 mL) was added tert-butyl (4-aminocyclohexyl)carbamate (268.82 mg, 1.25 mmol), Pd₂(dba)₃ (144.87 mg, 0.13 mmol), Ruphos (117 mg, 0.25 mmol) and Cs₂CO₃ (817.42 mg, 2.5 mmol). Then the mixture was stirred 16 hours at 90° C. It was quenched by H₂O (300 mL) and extracted with EA (3×300 mL).

The organic layers were combined, washed with brine NaCl (2×300 mL) and dried with MgSO₄, filtered and concentrated. The crude product was applied onto a silica gel column (24 g) eluted with PE: EA (10:1) to give product (200 mg, yield: 38.5%) as a white solid. Mass (m/z): 373.3 [M+H]⁺.

Step 3. Preparation of 1-N-(5,5-dimethyl-5,6,7,8-tetrahydronaphthalen-2-yl)cyclo-hexane-1,4-diamine (337)

To a solution of 337-2 (200 mg, 0.54 mmol) in 1,4-dioxane (5 mL) was added 4 N HCl in 1,4-dioxane (10 mL). Then the mixture was stirred overnight at 25° C. The reaction was concentrated under vacuum. The residue was purified by prep-TLC to afford the desired products 337A (56.1 mg) as a white solid and 337B (24.7 mg) as a white solid. 337A: ¹H NMR (400 MHZ, CD₃OD) δ 7.47 (dd, J=8.4, 5.2 Hz, 1H), 7.07 (d, J=8.4 Hz, 1H), 6.97 (d, J=9.8 Hz, 1H), 3.44 (m, 1H), 3.11 (m, 1H), 2.92-2.70 (m, 2H), 2.11 (d, J=10.0 Hz, 4H), 1.87-1.76 (m, 2H), 1.71-1.64 (m, 2H), 1.58-1.41 (m, 4H), 1.27-1.24 (m, 6H). Mass (m/z): 373.0 [M+H]⁺. HPLC: Rt=4.141 min (Column: XBRIDGE 3.5 μm, 2.1*50 mm, Mobile phase: H₂O (0.05% TFA)-ACN (0.05% TFA), ACN from 0 to 60% over 7 minutes, 7-8 min, ACN from 60% to 100%, Flow rate: 0.8 mL/min). 337B: ¹H NMR (400 MHZ, CD₃OD) δ 7.47 (d, J=8.4 Hz, 1H), 7.17-7.08 (m, 1H), 7.02 (s, 1H), 3.59 (s, 1H), 3.39 (s, 1H), 2.77 (t, J=6.2 Hz, 2H), 2.03-1.77 (m, 10H), 1.72-1.59 (m, 2H), 1.25 (d, J=14.8 Hz, 6H). Mass (m/z): 373.0 [M+H]⁺. HPLC: Rt=4.266 min (Column: XBRIDGE 3.5 μm, 2.1*50 mm, Mobile phase: H₂O (0.05% TFA)-ACN (0.05% TFA), ACN from 0 to 60% over 7 minutes, 7-8 min, ACN from 60% to 100%, Flow rate: 0.8 mL/min).

### Compound 338

### N-(((1r,4r)-4-aminocyclohexyl)methyl)-5,5,8,8-tetramethyl-5,6,7,8-tetrahydronaphthalen-2-amine

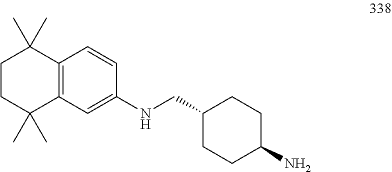 [SMILES: CC1(C)CCC(C)(C)c2cc(NC[C@H]3CC[C@H](N)CC3)ccc21]

The title compound 338 (25.7 mg, 21.1%) as yellow oil was prepared from tert-butyl ((1r,4r)-4-(((5,5,8,8-tetramethyl-5,6,7,8-tetrahydronaphthalen-2-yl)amino)methyl)cyclohexyl) c arbamate, DCM (6 mL) and 2,2,2-trifluoroacetic acid (3 mL) according to the procedure for 24. 1¹H NMR (400 MHZ, DMSO-d₆) δ 7.79-7.59 (m, 3H), 6.96 (d, J=8.5 Hz, 1H), 6.46-6.26 (m, 2H), 2.91 (d, J=5.0 Hz, 1H), 2.78 (d, J=6.5 Hz, 2H), 1.97-1.77 (m, 4H), 1.54 (d, J=2.1 Hz, 4H), 1.42 (s, 1H), 1.30-1.17 (m, 2H), 1.15 (s, 6H), 1.12 (s, 6H), 1.04-0.90 (m, 2H). Mass (m/z): 315.3 [M+H]⁺.

### Compound 339

### N-(4-(aminomethyl)-4-methylcyclohexyl)-4-(tert-butyl)aniline

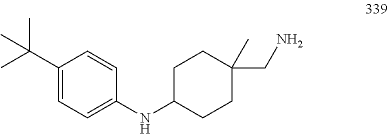 [SMILES: CC1(CN)CCC(Nc2ccc(C(C)(C)C)cc2)CC1]

The title compound 339 (57.1 mg) was prepared in a two-step overall yield of 44.9% as a white powder from 4-(tert-butyl)aniline (70 mg, 0.47 mmol) and 1-methyl-4-oxocyclohexane-1-carbonitrile (77 mg, 0.56 mmol) according to the procedure for 84. ¹H NMR (300 MHz, DMSO-d₆) δ 7.81 (s, 3H), 7.06 (d, J=8.0 Hz, 2H), 6.49 (s, 2H), 3.17 (d, J=12.1 Hz, 1H), 2.75 (d, J=5.7 Hz, 2H), 1.84-1.53 (m, 5H), 1.43 (s, 1H), 1.27 (s, 2H), 1.19 (s, 9H), 0.96 (d, J=5.1 Hz, 3H). Mass (m/z): 275.3 [M+H]⁺.

### Compound 340

### N-((3-(aminomethyl)cyclopentyl)methyl)-4-(tert-butyl)aniline

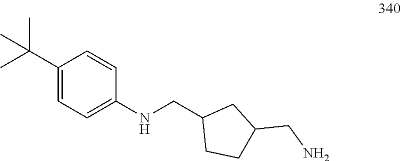 [SMILES: CC(C)(C)c1ccc(NCC2CCC(CN)C2)cc1]

The title compound 340 (57.1 mg) was prepared in a two-step overall yield of 44.9% as a white powder from 4-(tert-butyl)aniline (100 mg, 0.67 mmol) and cyclopentane-1,3-dicarboxylic acid (318 mg, 2.01 mmol) according to the procedure for 84. ¹H NMR (400 MHz, DMSO-d₆) δ 7.98 (s, 2H), 7.13-7.00 (m, 2H), 6.54-6.41 (m, 2H), 5.36 (s, 1H), 2.89 (dd, J=7.0, 4.6 Hz, 2H), 2.74 (d, J=7.2 Hz, 2H), 2.24-2.04 (m, 2H), 2.00 (dt, J=13.8, 7.0 Hz, 1H), 1.82-1.63 (m, 2H), 1.41-1.27 (m, 2H), 1.19 (s, 9H), 0.89 (dt, J=12.5, 9.6 Hz, 1H). Mass (m/z): 261.4 [M+H]⁺.

### Compound 341

### (2R, 3R)-3-amino-4-((4-((4-(tert-butyl)phenyl)amino)cyclohexyl)amino) butan-2-ol

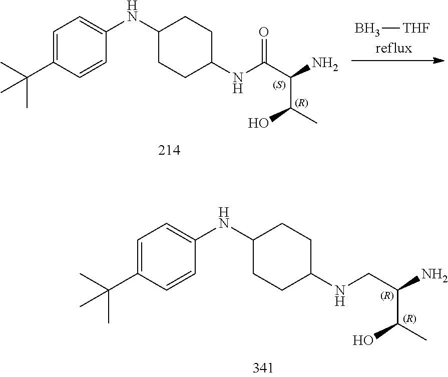

To a solution of (2S,3R)-2-amino-N-(4-((4-(tert-butyl)phenyl)amino)cyclohexyl)-3-hydroxybutanamide (230 mg, 0.66 mmol) in THF (5 mL) was added BH₃-THF (10 mL). The reaction was stirred at 70° C. for 18 hours. The solids were filtered and solvent was removed under vacuum. The residue was purified by prep-HPLC (column-Xbridge-C18 150×19 mm, 5 μm; Mobile phase: ACN-H₂O (0.1% FA), 25%-40%) to afford 341 (80.2 mg) as a white solid. ¹H NMR (400 MHZ, DMSO-d₆) δ 8.29 (s, 2H), 7.06 (d, J=8.6 Hz, 2H), 6.48 (d, J=8.6 Hz, 2H), 3.73-3.63 (m, 1H), 3.09 (t, J=10.4 Hz, 1H), 2.83 (t, J=9.6 Hz, 2H), 2.63 (m, 1H), 1.96 (s, 4H), 1.28 (d, J=9.4 Hz, 1H), 1.20 (s, 9H), 1.14 (dd, J=18.6, 6.8 Hz, 6H). Mass (m/z): 334.3 [M+H]⁺.

### Compound 342

### N-(((1r,4r)-4-aminocyclohexyl)methyl)-4-(tert-pentyl)aniline

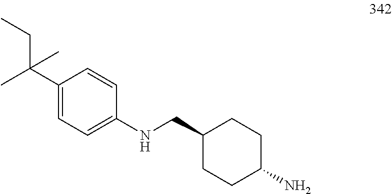 [SMILES: CCC(C)(C)c1ccc(NC[C@H]2CC[C@H](N)CC2)cc1]

The title compound 342 (7.5 mg) was prepared in a total yield of 14.2% as a white solid from tert-butyl ((1r,4r)-4-(((4-(tert-pentyl)phenyl)amino)methyl)cyclohexyl)carbamate (72 mg, 0.193 mmol), TFA (1 mL) and DCM (10 mL) according to the procedure for 24. ¹H NMR (400 MHz, DMSO-d₆) δ 7.84 (s, 3H), 7.00 (d, J=8.4 Hz, 2H), 6.48 (d, J=8.4 Hz, 2H), 5.44 (q, J=8.8, 5.6 Hz, 1H), 2.94-2.74 (m, 3H), 2.02-1.79 (m, 4H), 1.52 (q, J=7.2 Hz, 3H), 1.30 (qd, J=12.8, 3.6 Hz, 2H), 1.16 (s, 5H), 1.07-0.91 (m, 2H), 0.60 (t, J=7.2 Hz, 3H). Mass (m/z): 275.3 [M+H]⁺.

### Compound 343

### N1-(1,3,3-trimethylindolin-6-yl)cyclohexane-1,4-diamine

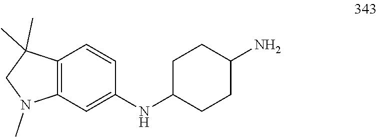 [SMILES: CN1CC(C)(C)c2ccc(NC3CCC(N)CC3)cc21]

The title compound 343 (21.7 mg) as a yellow solid was prepared from tert-butyl (4-((1,3,3-trimethylindolin-6-yl)amino)cyclohexyl)carbamate (200 mg, 0.53 mmol), 1,4-dioxane (5 mL) and HCl/1,4-dioxane (5 mL) according to the procedure for 37. ¹H NMR (400 MHZ, CD₃OD) δ 6.68-6.62 (m, 1H), 6.02-5.93 (m, 1H), 5.85 (dd, J=13.8, 1.8 Hz, 1H), 3.34 (s, 1H), 3.21 (dt, J=3.2, 1.6 Hz, 2H), 2.75-2.66 (m, 1H), 2.62-2.52 (m, 3H), 1.59 (tdd, J=11.6, 10.4, 4.8 Hz, 6H), 1.42 (dt, J=10.8, 7.0 Hz, 2H), 1.12 (s, 6H). Mass (m/z): 274.3 [M+H]⁺.

### Compound 344

### 1-((4-((4-(tert-butyl)phenyl)amino)cyclohexyl)methyl)guanidine

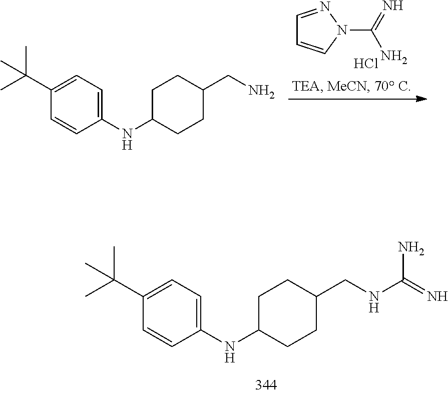 [SMILES: CC(C)(C)c1ccc(NC2CCC(CN)CC2)cc1 ; N=C(N)n1cccn1 ; Cl ; CC(C)(C)c1ccc(NC2CCC(CNC(=N)N)CC2)cc1]

A 10-mL round-bottom flask was charged with N-(4-(aminomethyl)cyclohexyl)-4-(tert-butyl)aniline (120 mg, mmol), 0.46 1H-pyrazole-1-carboximidamide hydrochloride (76 mg, 0.69 mmol), TEA (93 mg, 0.92 mmol) and 1,4-dioxane (5 mL). The reaction was stirred for 18 hours at 70° C. The reaction mixture was filtered and the filtrate was concentrated. The residue was purified by prep-HPLC (column-Xbridge-C18 150×19 mm, 5 μm; Mobile phase: ACN-H₂O (0.1% FA), 25%-40%) to afford 344 (18.3 mg) as a white solid. ¹H NMR (400 MHZ, DMSO-d₆) δ 8.76 (s, 1H), 8.40 (s, 1H), 7.73 (s, 2H), 7.07 (d, J=8.8 Hz, 2H), 6.53 (d, J=8.8 Hz, 2H), 3.45 (s, 1H), 2.98 (t, J=5.8 Hz, 2H), 1.68-1.39 (m, 9H), 1.20 (s, 9H). Mass (m/z): 303.3 [M+H]⁺.

Compound 345

### (1s,3R,5S)-N1-(4-(tert-butyl)phenyl)cyclohexane-1,3,5-triamine

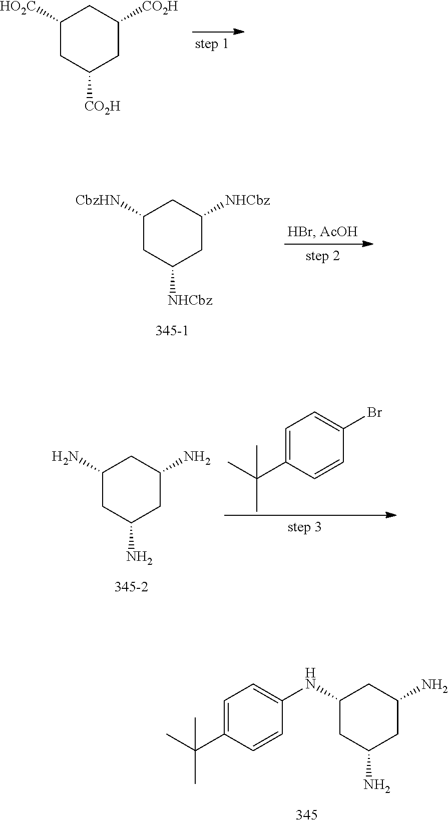

Step 1. Preparation of (1s,3s,5s)-cyclohexane-1,3,5-triamine (345-1)

Add cis-1,3,5-cyclohexane tricarboxylic acid (1.0 g, 4.6 mmol), triethylamine (1.4 g, 13.8 mmol), and diphenyl phosphoryl azide (3.8 g, 13.8 mmol) in toluene (30 mL), then stirred at room temperature for 30 minutes. The mixture was stirred at 80° C. for 90 minutes, then add benzyl alcohol (1.99 g, 18.4 mmol) to the mixture, reflux the solution for 24 hours. Cool the solution to ambient temperature. Collect the white precipitate by filtration, wash the precipitate with cold toluene to give the product (1.7 g, 69.5%). Mass (m/z): 532.3 [M+H]⁺.

Step 2. Preparation of (1s,3s,5s)-cyclohexane-1,3,5-triamine (345-2)

Dissolve tris-benzyl carbamate (1.4 g, 2.63 mmol) in 33% HBr/glacial acetic acid (5 mL). Allow the mixture to stand for 90 minutes under stirring. Add diethyl ether (100 mL) to the mixture, filter off the product, wash the product with diethyl ether to give the product (300 mg, 88.2%). Mass (m/z): 130.1 [M+H]⁺.

Step 3. Preparation of (1s,3R,5S)-N1-(4-(tert-butyl)phenyl)cyclohexane-1,3,5-triamine (345)

Pd₂(dba)₃ (43 mg, 0.047 mmol), Cs₂CO₃ (611 mg, 1.88 mmol), and RuPhos (22 mg, 0.047 mmol) were added to a solution of cyclohexane-1,3,5-triamine trihydrobromide (174 mg, 0.47 mmol), 1-bromo-4-tert-butylbenzene (50 mg, 0.23 mmol) in toluene (10 mL), and the mixture was heated to 110° C. for 16 hrs, then cooled to rt, the solvent was removed in vacuo. The residue was purified by prep-HPLC (column-Gemini-C18 150×21.2 mm, 5 μm; Mobile phase: ACN-H₂O (0.1% FA), 5%-20%) to afford the desired product 345 as a yellow solid (12.2 mg, 19.9%). ¹H NMR (400 MHZ, DMSO-d₆) δ 7.16 (d, J=8.5 Hz, 2H), 6.63 (d, J=8.3 Hz, 2H), 3.47 (s, 1H), 3.17 (s, 1H), 2.54 (s, 2H), 2.22 (d, J=10.8 Hz, 3H), 1.45-1.37 (m, 1H), 1.22 (s, 9H), 1.19 (s, 1H). Mass (m/z): 262.3 [M+H]⁺.

### Compound 346

N-((4-((4-(tert-butyl)phenyl)amino)cyclohexyl)methyl)-1-methyl-1H-pyrazol-4-amine

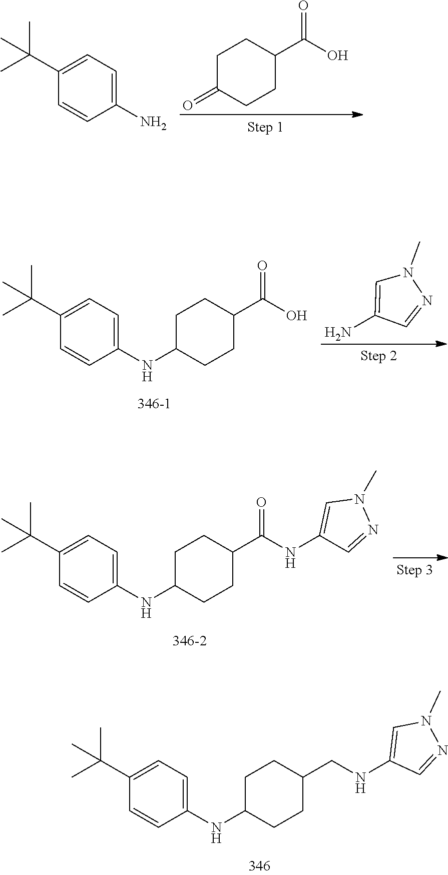

Step 1. Preparation of 4-((4-(tert-butyl)phenyl)amino)cyclohexane-1-carboxylic acid (346-1)

A mixture of 4-(tert-butyl)aniline (1 g, 0.0067 mmol), 4-oxocyclohexane-1-carboxylic acid (1.14 g, 0.0080 mmol) and NaBH₃CN (0.84 g, 0.0134 mmol) in MeOH (10 mL) and HOAc (1 drop) was stirred overnight at 50° C. After cooling, the excess MeOH was removed under vacuum and the residual was diluted with water (20 mL), extracted three times with ethyl acetate (20 mL). Organic layers were combined, solvent was removed under vacuum and the crude was purified through silica gel chromatography (PE:EA=3:1) to give the desired product as an oil (1.5 g, 49.2%). Mass (m/z): 276.3 [M+H]⁺.

Step 2. Preparation of 4-((4-(tert-butyl)phenyl)amino)-N-(1-methyl-1H-pyrazol-4-yl)cyclohexane-1-carboxamide (346-2)

A mixture of 4-((4-(tert-butyl)phenyl)amino)cyclohexane-1-carboxylic acid (100 mg, 0.3631 mmol), 1-methyl-1H-pyrazol-4-amine (42.3 mg, 0.4357 mmol), HATU (207.1 mg, 0.5447 mmol) and DIEA (140.8 mg, 1.0893 mmol) in DMF (10 mL) was stirred overnight at rt. Then the reaction solution was diluted with water (30 mL), extracted three times with ethyl acetate (20 mL). Organic layers were combined, solvent was removed under vacuum and the crude was purified through silica gel chromatography (PE/EA=2:1) to give the desired product as an oil (200 mg, 77.7%). Mass (m/z): 355.5 [M+H]⁺.

Step 3. Preparation of N-((4-((4-(tert-butyl)phenyl)amino)cyclohexyl)methyl)-1-methyl-1H-pyrazol-4-amine (346)

To a solution of N-((4-((4-(tert-butyl)phenyl)amino)cyclohexyl)methyl)-1-methyl-1H-pyrazol-4-amine (200 mg, 0.5642 mmol) in THF (1 mL) was added BH₃-THF (30 mL). Then the mixture was stirred overnight at 70° C. After the reaction was completed, solvent was removed under vacuum and the residue was purified by prep-HPLC (column-Xbridge-C18 150×21.2 mm, 5 μm; Mobile phase: ACN-H₂O (0.1% FA), 2%-20%) to afford the desired product 346 (6 mg, 3%) as a white solid. ¹H NMR (400 MHZ, CD₃OD) δ 7.15 (dd, J=11.9, 9.2 Hz, 4H), 6.65 (d, J=8.6 Hz, 2H), 3.79 (s, 3H), 3.53 (s, 1H), 2.89 (d, J=6.8 Hz, 2H), 1.74-1.44 (m, 9H), 1.28 (s, 9H). Mass (m/z): 341.2 [M+H]⁺.

### Compound 347

### N1-(4-(tert-butyl)phenyl)cyclohexane-1,2,2,6,6-d5-1,4-diamine

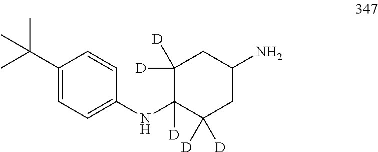 [SMILES: [2H]C1([2H])CC(N)CC([2H])([2H])C1([2H])Nc1ccc(C(C)(C)C)cc1]

The title compound 347 (105.7 mg) was prepared in a total yield of 83.9% as a white solid from tert-butyl (4-((4-(tert-butyl)phenyl)amino)cyclohexyl-3,3,4,5,5-d5) carbamate (176 mg, 0.5 mmol), and TFA according to the procedure for compound 24. ¹H NMR (400 MHZ, Methanol-d₄) δ 7.16 (d, J=8.4 Hz, 2H), 6.61 (d, J=8.4 Hz, 2H), 3.19-2.95 (m, 1H), 2.07-1.98 (m, 1H), 1.85-1.67 (m, 2H), 1.52-1.40 (m, 1H), 1.25 (s, 9H). Mass (m/z): 252.3 [M+H]⁺.

### Compound 348

### N-((4-((4-(tert-butyl)phenyl)amino)cyclohexyl)methyl)-1H-pyrazol-4-amine

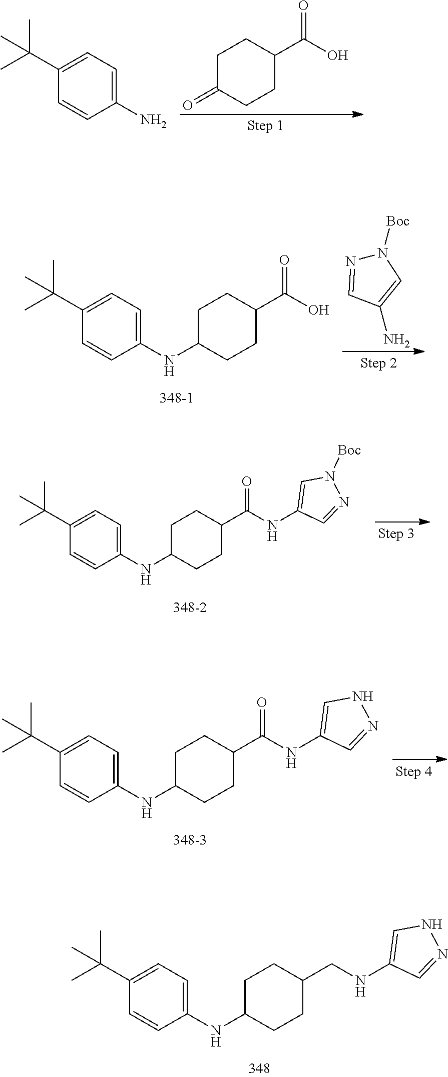

Step 1. Preparation of 4-((4-(tert-butyl)phenyl)amino)cyclohexane-1-carboxylic acid (348-1) A mixture of 4-(tert-butyl)aniline (1 g, 0.0067 mmol), 4-oxocyclohexane-1-carboxylic acid (1.14 g, 0.0080 mmol) and NaBH₃CN (0.84 g, 0.0134 mmol) in MeOH (10 mL) and HOAc (1 drop) was stirred overnight at 50° C. After cooling, the excess MeOH was removed under vacuum and the residue was diluted with water (20 mL), extracted three times with ethyl acetate (20 mL). Organic layers were combined, solvent was removed under vacuum and the crude was purified through silica gel chromatography (PE:EA=3:1) to give the desired product as oil (1.5 g, 49.2%). Mass (m/z): 276.3 [M+H]⁺.

Step 2. tert-butyl 4-(4-((4-(tert-butyl)phenyl)amino)cyclohexane-1-carboxamido)-1H-pyrazole-1-carboxylate (348-2)

A mixture of 4-((4-(tert-butyl)phenyl)amino)cyclohexane-1-carboxylic acid (300 mg, 1.0894 mmol), tert-butyl 4-amino-1H-pyrazole-1-carboxylate (301.03 mg, 1.6341 mmol), EDCI (313.26 mg, 1.6341 mmol), DIEA (281.59 mg, 2.1788 mmol) and HOBT (220.80 mg, 1.6341 mmol) in DMF (10 mL) was stirred overnight at rt. Then the reaction solution was diluted with water (30 mL), extracted three times with ethyl acetate (20 mL). Organic layers were combined, solvent was removed under vacuum and the crude was purified through silica gel chromatography (PE/EA=5:1) to give the desired product as oil (376 mg, 46.4%). Mass (m/z): 441.5 [M+H]⁺.

Step of 3. Preparation 4-((4-(tert-butyl)phenyl)amino)-N-(1H-pyrazol-4-yl)cyclohexane-1-carboxamide (348-3)

The mixture of 4-(4-((4-(tert-butyl)phenyl)amino)cyclohexane-1-carboxamido)-1H-pyrazole-1-carboxylate (376 mg, 0.8515 mmol) in 1,4-dioxane (10 mL) and 1,4-dioxane/HCl (10 mL) was stirred for 16 hours at 25° C. After the reaction was completed, the solvent was removed under vacuum to give the desired product as oil (200 mg, 38.6%). Mass (m/z): 340.9 [M+H]⁺.

Step 4. Preparation of N-((4-((4-(tert-butyl)phenyl)amino)cyclohexyl)methyl)-1H-pyrazol-4-amine (348)

To a solution of N-((4-((4-(tert-butyl)phenyl)amino)cyclohexyl)methyl)-1-methyl-1H-pyrazol-4-amine (200 mg, 0.5874 mmol) in THF (1 mL) was added BH₃-THF (30 mL). Then the mixture was stirred overnight at 25° C. After the reaction was completed, solvent was removed under vacuum and the residue was purified by prep-HPLC (column-Xbridge-C18 150×19 mm, 5 μm; Mobile phase: ACN-H₂O (0.05% NH₃), 55%-60%) to afford the desired product 348 (9.7 mg, 5.0%) as a purple solid. ¹H NMR (400 MHZ, DMSO-d₆) δ 12.00 (s, 1H), 7.06 (d, J=8.6 Hz, 2H), 7.01 (s, 2H), 6.47 (d, J=8.6 Hz, 2H), 5.10 (d, J=7.8 Hz, 1H), 4.26 (brs, 1H), 3.08 (brs, 1H), 2.69 (d, J=6.5 Hz, 2H), 1.98 (m, 2H), 1.84 (m, 2H), 1.47 (brs, 1H), 1.20 (s, 9H), 1.05 (q, J=10.9 Hz, 4H). Mass (m/z): 327.5 [M+H]⁺.

### Compound 349

### 1-amino-3-((4-((4-(tert-butyl)phenyl)amino)cyclohexyl)amino)propan-2-ol

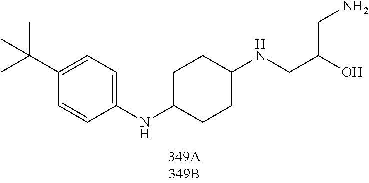 [SMILES: CC(C)(C)c1ccc(NC2CCC(NCC(O)CN)CC2)cc1]

The title compound 349A (68.7 mg, 29.8%) as a yellow solid and compound 349B (62 mg, 27.4%) as a yellow solid were prepared from tert-butyl (3-((4-((4-(tert-butyl)phenyl)amino)cyclohexyl)amino)-2-hydroxypropyl)carbamate (300 mg, 0.68 mmol), and 4M HCl/dioxane (20 mL) according to the procedure for 37. 349A: ¹H NMR (400 MHZ, CD₃OD) δ 7.37 (d, J=8.7 Hz, 2H), 6.96 (d, J=8.6 Hz, 2H), 4.14 (tt, J=9.3, 3.3 Hz, 0H), 3.41-3.32 (m, 0H), 3.23-3.00 (m, 2H), 2.94 (d, J=8.6 Hz, 1H), 2.18 (s, 2H), 1.65-1.34 (m, 2H), 1.28 (s, 5H). Mass (m/z): 320.8 [M+H]⁺. HPLC: Rt: 2.996 min (Column: XBRIDGE 2.1*50 mm, 3.5 μm; Mobile Phase: H₂O (0.05% TFA), ACN (0.05% TFA), ACN from 0% to 60% over 7 minutes, 7-8 min, ACN from 60% to 100%; 0.8 mL/min). 349B: H NMR (400 MHZ, CD₃OD) δ 7.14 (d, J=8.7 Hz, 2H), 6.60 (d, J=8.7 Hz, 2H), 4.19-4.11 (m, 1H), 3.63-3.56 (m, 0H), 3.23-2.99 (m, 2H), 2.92 (dd, J=13.0, 8.6 Hz, 1H), 2.02-1.67 (m, 8H), 1.23 (d, J=1.7 Hz, 9H). Mass (m/z): 320.8 [M+H]⁺. HPLC: Rt: 3.380 min (Column: XBRIDGE 2.1*50 mm, 3.5 μm; Mobile Phase: H₂O (0.05% TFA) ACN (0.05% TFA), ACN from 0% to 60% over 7 minutes, 7-8 min, ACN from 60% to 100%; 0.8 mL/min).

### Compound 350

### N—((S)-1-((1r,4S)-4-aminocyclohexyl)ethyl)-4-(tert-butyl)aniline

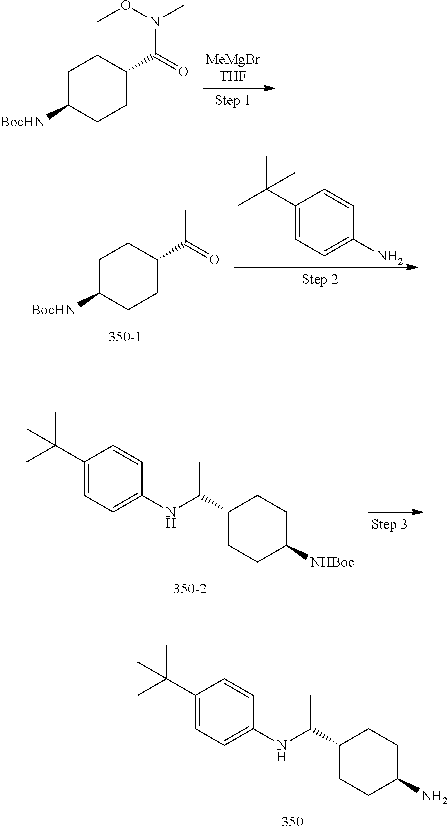 [SMILES: CON(C)C(=O)[C@H]1CC[C@H](NC(=O)OC(C)(C)C)CC1 ; CC(=O)[C@H]1CC[C@H](NC(=O)OC(C)(C)C)CC1 ; CC(C)(C)c1ccc(N)cc1 ; CC(Nc1ccc(C(C)(C)C)cc1)[C@H]1CC[C@H](NC(=O)OC(C)(C)C)CC1 ; CC(Nc1ccc(C(C)(C)C)cc1)[C@H]1CC[C@H](N)CC1]

Step 1. Preparation of tert-butyl ((1r,4r)-4-acetylcyclohexyl)carbamate (350-1)

A mixture of tert-butyl ((1r,4r)-4-(methoxy(methyl) carbamoyl)cyclohexyl)carbamate (500 mg, 1.746 mmol), and CH₃MgBr (1.46 mL, 4.365 mmol) in THF (10 mL) was stirred overnight at 0° C. under N₂. After cooling, diluted, extracted three times with ethyl acetate (20 mL). The organic layers were combined, solvent was removed under vacuum and the crude was purified through silica gel chromatography (PE:EA=2:1) to give the desired product as white solid (350 mg, 46.5%). Mass (m/z): 242.17 [M+H]⁺.

Step 2. Preparation of tert-butyl ((1S,4r)-4-((S)-1-((4-(tert-butyl)phenyl)amino)ethyl)cyclohexyl)carbamate (350-2)

A mixture of 4-(tert-butyl)aniline (200 mg, 1.3402 mmol), tert-butyl ((1r,4r)-4-acetylcyclohexyl)carbamate (485.4 mg, 2.0103 mmol) and NaBH (OAc) 3 (852.13 mg, 4.0206 mmol) in DCM (20 mL) was stirred overnight at 25° C. After cooling, the excess DCM was removed under vacuum and the residue was extracted three times with DCM (20 mL) and water (20 mL). Organic layer was combined, solvent was removed under vacuum and the crude was purified through silica gel chromatography (PE:EA=7:1) to give the desired product (150 mg, 14.9%) as oil. Mass (m/z): 375.3 [M+H]⁺.

Step 3. Preparation of N—((S)-1-((1r,4S)-4-aminocyclohexyl)ethyl)-4-(tert-butyl)aniline (350)

The crude product was purified by prep-HPLC (column-Gemini-C18 150×21.2 mm, 5 μm; Mobile phase: ACN-H₂O (0.1% FA), 10%-30%) to afford the desired product 350 (30.1 mg, 26.2%) as a white solid. ¹H NMR (400 MHz, DMSO-d₆) δ 7.05 (d, J=8.7 Hz, 2H), 6.46 (d, J=8.7 Hz, 2H), 5.10 (s, 1H), 3.18 (m, 1H), 2.81 (m, 1H), 2.00-1.69 (m, 4H), 1.39-1.22 (m, 2H), 1.20 (s, 10H), 1.13-1.03 (m, 2H), 1.01 (d, J=6.5 Hz, 3H). Mass (m/z): 275.3 [M+H]⁺.

### Compound 351

### (1R, 3S,5r)-N1,N3-bis(4-(tert-butyl)phenyl)cyclohexane-1,3,5-triamine

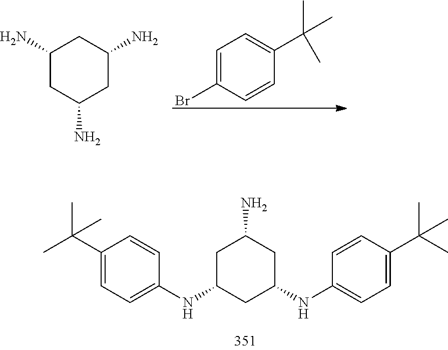 [SMILES: N[C@H]1C[C@@H](N)C[C@@H](N)C1 ; CC(C)(C)c1ccc(Br)cc1 ; CC(C)(C)c1ccc(N[C@@H]2C[C@H](N)C[C@H](Nc3ccc(C(C)(C)C)cc3)C2)cc1]

Pd₂(dba)₃ (49 mg, 0.054 mmol), Cs₂CO₃ (701 mg, 2.15 mmol), and RuPhos (25 mg, 0.054 mmol) were added to a solution of cyclohexane-1,3,5-triamine trihydrobromide, and 1-bromo-4-tert-butylbenzene (100 mg, 0.27 mmol) in toluene (10 mL), and the mixture was heated to 110° C. for 16 hrs, then cooled to room, the solvent was removed in vacuo, the residue was purified by prep-HPLC (column-Gemini-C18 150×21.2 mm, 5 μm; Mobile phase: ACN-H₂O (0.1% FA), 5%-20%) to afford the desired product as yellow solid (8 mg, 7.5%). ¹H NMR (400 MHZ, CD₃OD) δ 7.23 (d, J=8.4 Hz, 4H), 6.73 (d, J=7.6 Hz, 4H), 3.58 (brs, 2H), 3.37 (brs, 2H), 2.68 (s, 1H), 2.45 (d, J=11.5 Hz, 4H), 1.28 (s, 16H). Mass (m/z): 394.4 [M+H]⁺.

### Compound 352

### N-(4-(4-aminocyclohexyl)phenyl)-3,4-bis(2-methoxyethyl)aniline

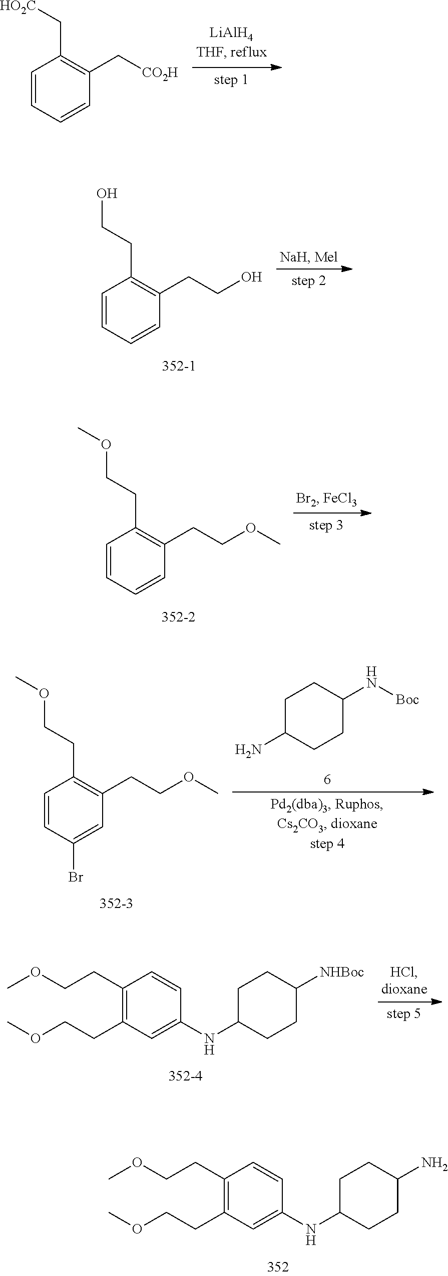

Step 1. Preparation of 2,2′-(1,2-phenylene) bis(ethan-1-ol) (352-1) A mixture of 2,2′-(1,2-phenylene) diacetic acid (5 g, 25.7 mmol) in THF (80 mL) was added LiAlH₄ (3.9 g, 10.28 mmol) at 25° C. The reaction was stirred at 70° C. for 16 h. The reaction was quenched with water. The mixture was diluted with EA (20 mL) and washed with water (20 mL×3). The organic phase was concentrated to give 352-1 as a pale solid (4 g, 84%). Mass (m/z): 167.2 [M+H]⁺.

Step 2. Preparation of 1,2-bis(2-methoxyethyl) benzene (352-2)

To a solution of 352-1 (4 g, 24.1 mmol) in THF (100 mL) was added NaH (1.21 g, 50.6 mmol) at 0° C. The reaction was stirred at 0° C. for 0.5 h. Then the reaction was added MeI (6.84 g, 48.2 mmol). The reaction was stirred at 25° C. for 2 h. The reaction was quenched with water. The mixture was diluted with EA (20 mL) and washed with water (20 mL×3). The organic phase was concentrated and purification by a silica gel column chromatography (PE:EA=4:1) to give 352-2 as a pale solid (3 g, 58%). Mass (m/z): 195.2 [M+H]⁺.

Step 3. Preparation of 4-bromo-1,2-bis(2-methoxyethyl) benzene (352-3)

To a solution of 353-2 (2 g, 10.3 mmol) in DCE (20 mL) was added FeCl₃ (5.01 g, 30.9 mmol) and Br₂ (3.29 g, 20.6 mmol) at 25° C. The reaction was stirred at 70° C. for 16 h. The reaction was quenched with water. The mixture was diluted with EA (20 mL), washed with water (20 mL×3) and washed with aq. Na₂S₂SO₃. The organic phase was concentrated and purification by a silica gel column chromatography (PE:EA=10:1) to give 353-3 as a white solid (0.5 g, 15.5%). Mass (m/z): 273.1 [M+H]⁺.

Step 4. Preparation of tert-butyl (4-((3,4-bis(2-methoxyethyl)phenyl)amino) cyclohexyl)carbamate (352-4)

A mixture of 353-3 (0.1 g, 0.4 mmol), tert-butyl (4-aminocyclohexyl)carbamate (85.6 mg, 0.4 mmol), Ruphos (0.04 g, 0.08 mmol), Cs₂CO₃ (260 mg, 0.8 mmol) and Pd₂(dba) 3 (36.6 mg, 0.04 mmol) in dioxane (2 mL) was stirred under nitrogen at 90° C. for 16 hrs. The mixture was filtered, concentrated and purification by Prep-TLC (PE:EA=1:1) to give 352-4 as a yellow solid (0.01 g, 5.6%). Mass (m/z): 406.8 [M+H]⁺.

Step 5. Preparation of N1-(3,4-bis(2-methoxyethyl)phenyl)cyclohexane-1,4-diamine (352)

To a solution of 352-4 (0.01 g, 0.0245 mmol) in DCM (5 mL) was added HCl in dioxane (1 mL). The reaction was stirred at 25° C. for 2 h. The mixture was concentrated and purification by Prep-TLC (DCM: MeOH-10:1) to give 352 (5 mg, 60%) as a yellow solid. ¹H NMR (300 MHz, CD₃OD) δ 7.25 (d, J-9 Hz, 1H), 7.12-6.98 (m, 1H), 6.96-6.76 (m, 1H), 3.75-3.30 (m, 8H), 3.29-2.66 (m, 6H), 2.12 (s, 2H), 1.85-1.49 (m, 8H). Mass (m/z): 307.3 [M+H]⁺.

### Compound 353

### N-(((1r,4r)-4-aminocyclohexyl)methyl)-4-isopropoxyaniline

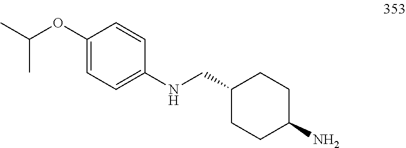 [SMILES: CC(C)Oc1ccc(NC[C@H]2CC[C@H](N)CC2)cc1]

The title compound 353 (26.1 mg, 17.0%) as white solid was prepared from tert-butyl ((11,4r)-4-(((4-isopropoxyphenyl)amino)methyl)cyclohexyl)carbamate (201 mg, 0.5777 mmol), DCM (10 mL) and TFA (1 mL) according to the procedure for 24. ¹H NMR (400 MHZ, CD₃OD) δ 6.76-6.70 (m, 2H), 6.62-6.56 (m, 2H), 4.35 (dt, J=12.1, 6.1 Hz, 1H), 2.87 (d, J=6.7 Hz, 2H), 2.56 (tt, J=10.9, 3.5 Hz, 1H), 1.88 (d, J=10.1 Hz, 4H), 1.58-1.47 (m, 1H), 1.24 (d, J=6.1 Hz, 6H), 1.15-0.98 (m, 4H). Mass (m/z): 263.20 [M+H]⁺.

### Compound 354

### (1r,4r)-N1-(4-(4-(trifluoromethyl)piperidin-1-yl)phenyl)cyclohexane-1,4-diamine

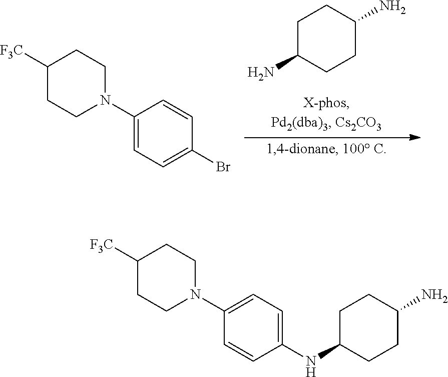 [SMILES: FC(F)(F)C1CCN(c2ccc(Br)cc2)CC1 ; N[C@H]1CC[C@H](N)CC1 ; N[C@H]1CC[C@H](Nc2ccc(N3CCC(C(F)(F)F)CC3)cc2)CC1]

A mixture of 1-(4-bromophenyl)-4-(trifluoromethyl)piperidine (46.1 mg, 0.15 mmol), (1r,4r)-cyclohexane-1,4-diamine (22.2 mg, 0.2 mmol), Pd₂(dba)₃ (4.6 mg, 5 μmol), X-phos (12 mg, 25 μmol), and Cs₂CO₃ (65.6 mg, 0.2 mmol) in 1,4-dioxane (3.0 mL) was stirred overnight at 100° C. under nitrogen atmosphere. After cooling to rt. 5 mL of water was added. The resulting solution was extracted with 3×10 mL of ethyl acetate. The organic layers were combined, washed with water (30 mL), dried and concentrated under vacuum. The residue was purified by prep-TLC (MeOH/DCM=1:5) to afford the desired product (16.1 mg, 31.4%) as a rosy brown solid. ¹H NMR (400 MHZ, DMSO-d₆) δ 6.81 (d, J=8.4 Hz, 2H), 6.61 (d, J=8.4 Hz, 2H), 3.55-3.42 (m, 2H), 3.07 (m, 1H), 2.94 (m, 1H), 2.68-2.55 (m, 2H), 2.16-1.96 (m, 4H), 1.93-1.77 (m, 4H), 1.58 (m, 1H), 1.54-1.36 (m, 2H), 1.27-1.11 (m, s 2H). Mass (m/z): 342.3 [M+H]⁺.

### Compound 355

### (1s,4s)-N1-(4-(4-(trifluoromethyl)piperidin-1-yl)phenyl)cyclohexane-1,4-diamine

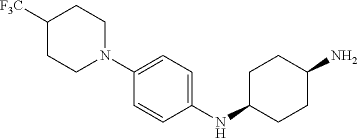 [SMILES: N[C@H]1CC[C@@H](Nc2ccc(N3CCC(C(F)(F)F)CC3)cc2)CC1]

The title compound 355 (26.1 mg) was prepared in a total yield of 50.8% as a rosy brown solid from 1-(4-bromophenyl)-4-(trifluoromethyl)piperidine (46.1 mg, 0.15 mmol), (1s,4s)-cyclohexane-1,4-diamine (22.2 mg, 0.2 mmol) according to the procedure for compound 354. ¹H NMR (400 MHz, DMSO-d₆) δ 6.76 (d, J=8.4 Hz, 2H), 6.55 (d, J=8.4 Hz, 2H), 3.50-3.37 (m, 2H), 3.33 (m, 1H), 3.06 (m, 1H), 2.62-2.53 (m, 2H), 1.92-1.83 (m, 2H), 1.80-1.67 (m, 6H), 1.64-1.48 (m, 5H). Mass (m/z): 342.3 [M+H]⁺.

### Compound 356

### N-(((1r,4r)-4-aminocyclohexyl)methyl)-4-(4-(trifluoromethyl)piperidin-1-yl)aniline

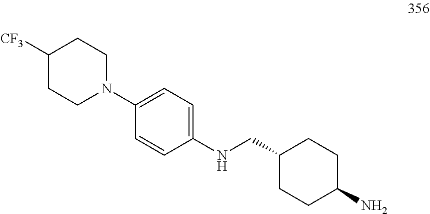 [SMILES: N[C@H]1CC[C@H](CNc2ccc(N3CCC(C(F)(F)F)CC3)cc2)CC1]

The desired product (29.4 mg, 47.7%) as a purple solid was prepared from tert-butyl ((1r,4r)-4-(((4-(4-(trifluoromethyl)piperidin-1-yl)phenyl)amino)methyl)cyclohexyl)carbamate (79 mg, 0.174 mmol), DCM (10 mL) and TFA (1 mL) according to the procedure for 24. ¹H NMR (400 MHZ, DMSO-d₆) δ 8.26 (s, 3H), 6.79 (s, 2H), 6.53 (s, 2H), 3.42 (s, 2H), 2.84 (d, J=33.6 Hz, 3H), 2.05-1.79 (m, 7H), 1.51 (d, J=38.4 Hz, 4H), 1.32 (d, J=13.6 Hz, 2H), 0.99 (s, 3H). Mass (m/z): 356.3 [M+H]⁺.

### Compound 357

### N2-(4-(4-(trifluoromethyl)piperidin-1-yl)phenyl)spiro[3.3]heptane-2, 6-diamine

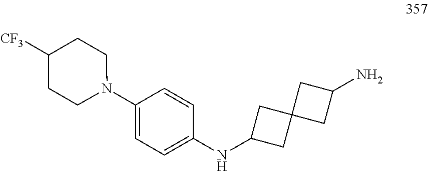 [SMILES: NC1CC2(C1)CC(Nc1ccc(N3CCC(C(F)(F)F)CC3)cc1)C2]

The title compound 357 (47.0 mg) was prepared in a yield of 55.9% as a white powder with 1:0. 2 mixture by ¹H NMR from 4-(4-(trifluoromethyl)piperidin-1-yl)aniline (54.2 mg, 0.22 mmol) and tert-butyl (6-oxospiro [3.3]heptan-2-yl)carbamate (60 mg, 0.27 mmol) according to the procedure for 20. ¹H NMR (400 MHZ, DMSO-d₆) δ 8.21 (s, 2H), 6.74 (d, J=8.7 Hz, 2H), 6.49-6.32 (m, 2H), 5.37 (d, J=6.8 Hz, 1H), 3.63 (q, J=7.2 Hz, 1H), 3.54 (p, J=8.1 Hz, 1H), 3.42 (d, J=11.9 Hz, 2H), 2.55 (d, J=2.5 Hz, 3H), 2.42-2.27 (m, 3H), 2.25-2.09 (m, 3H), 1.91-1.70 (m, 4H), 1.56 (qd, J=12.5, 4.0 Hz, 2H). Mass (m/z): 354.3 [M+H]⁺.

### Compound 358

### N2-(2-(4-isopropylpiperidin-1-yl)pyrimidin-5-yl)spiro[3.3]heptane-2,6-diamine

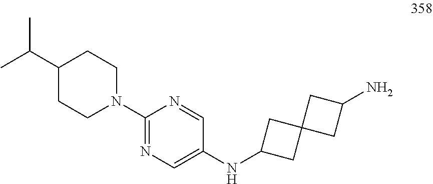 [SMILES: CC(C)C1CCN(c2ncc(NC3CC4(CC(N)C4)C3)cn2)CC1]

The title compound 358 (55.2 mg) was prepared in a yield of 75.4% as a white powder with 1:0. 2 mixture by ¹H NMR from 2-(4-isopropylpiperidin-1-yl)pyrimidin-5-amine (49.3 mg, 0.22 mmol) and tert-butyl (6-oxospiro [3.3]heptan-2-yl)carbamate (60 mg, 0.27 mmol) according to the procedure for 20. ¹H NMR (400 MHZ, DMSO-d₆) δ 8.11 (s, 3H), 7.79 (s, 2H), 5.29 (d, J=7.4 Hz, 1H), 4.47 (dt, J=12.7, 2.8 Hz, 2H), 3.63 (p, J=7.4 Hz, 1H), 3.52 (p, J=8.1 Hz, 1H), 2.61 (td, J=12.6, 2.5 Hz, 2H), 2.41-2.28 (m, 2H), 2.15 (ddt, J=11.5, 8.4, 4.5 Hz, 3H), 1.79 (dt, J=11.3, 8.2 Hz, 2H), 1.63 (dd, J=12.9, 3.2 Hz, 2H), 1.38 (hept, J=6.6 Hz, 1H), 1.26-1.14 (m, 1H), 1.06 (qd, J=12.4, 4.2 Hz, 2H), 0.84 (d, J=6.8 Hz, 6H). Mass (m/z): 330.3 [M+H]⁺.

### Compound 359

### N2-(2-(4-(trifluoromethyl)piperidin-1-yl)pyrimidin-5-yl)spiro[3.3]heptane-2,6-diamine

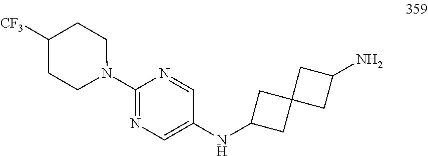 [SMILES: NC1CC2(C1)CC(Nc1cnc(N3CCC(C(F)(F)F)CC3)nc1)C2]

The title compound 359 (17.2 mg) was prepared in a yield of 21.8% as a white powder with 1:0. 2 mixture by ¹H NMR from 2-(4-(trifluoromethyl)piperidin-1-yl)pyrimidin-5-amine (55.1 mg, 0.22 mmol) and tert-butyl (6-oxospiro [3.3]heptan-2-yl)carbamate (60 mg, 0.27 mmol) according to the procedure for 20. ¹H NMR (400 MHZ, DMSO-d₆) δ 8.24 (s, 3H), 7.82 (s, 2H), 5.41 (d, J=7.2 Hz, 1H), 4.65-4.44 (m, 2H), 3.64 (h, J=7.4 Hz, 1H), 3.53 (p, J=8.1 Hz, 1H), 2.76 (td, J=12.9, 2.5 Hz, 2H), 2.56 (td, J=8.7, 3.8 Hz, 1H), 2.42-2.28 (m, 2H), 2.26-2.12 (m, 3H), 1.81 (td, J=11.5, 9.6, 5.9 Hz, 4H), 1.34 (qd, J=12.6, 4.2 Hz, 2H). Mass (m/z): 356.2 [M+H]⁺.

### Compound 360

### N-(((1r,4r)-4-aminocyclohexyl)methyl)-2-(4-isopropylpiperidin-1-yl)pyrimidin-5-amine

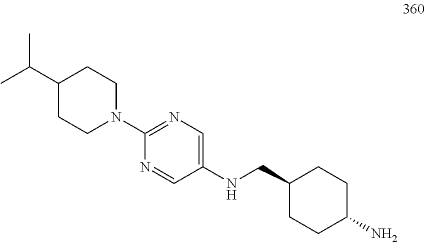 [SMILES: CC(C)C1CCN(c2ncc(NC[C@H]3CC[C@H](N)CC3)cn2)CC1]

The desired product 360 as a white solid (38.8 mg, 42.3%) was prepared from tert-butyl ((1r,4r)-4-(((2-(4-isopropylpiperidin-1-yl)pyrimidin-5-yl)amino)methyl)cyclohexyl)carbamate (crude), DCM (6 mL) and 2,2,2-trifluoroacetic acid (3 mL) according to the procedure for 24. ¹H NMR (400 MHZ, DMSO-d₆) δ 7.86 (s, 2H), 5.05 (t, J=6.0 Hz, 1H), 4.47 (d, J=12.9 Hz, 2H), 2.78 (t, J=6.4 Hz, 2H), 2.70-2.54 (m, 4H), 1.77 (d, J=9.8 Hz, 4H), 1.64 (d, J=12.8 Hz, 2H), 1.40 (dd, J=13.0, 6.6 Hz, 2H), 1.26-0.91 (m, 7H), 0.85 (d, J=6.8 Hz, 6H). Mass (m/z): 332.3 [M+H]⁺

### Compound 361

### N-(((1s,4s)-4-aminocyclohexyl)methyl)-2-(4-isopropylpiperidin-1-yl)pyrimidin-5-amine

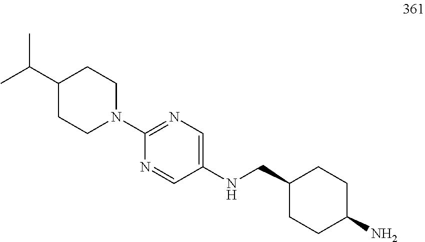 [SMILES: CC(C)C1CCN(c2ncc(NC[C@H]3CC[C@@H](N)CC3)cn2)CC1]

The desired product 361 (10.8 mg, 11.9%) as a yellow solid was prepared from tert-butyl ((1s,4s)-4-(((2-(4-isopropylpiperidin-1-yl)pyrimidin-5-yl)amino)methyl)cyclohexyl)carbamate according to the procedure for 24. ¹H NMR (400 MHZ, DMSO-d₆) δ 7.99 (s, 1H), 7.87 (d, J=4.9 Hz, 1H), 5.07 (t, J=5.9 Hz, 1H), 4.57-4.41 (m, 2H), 2.97-2.79 (m, 2H), 2.70-2.56 (m, 2H), 2.41-2.34 (m, 1H), 1.82-1.56 (m, 3H), 1.43 (dt, J=13.2, 10.2 Hz, 6H), 1.17 (d, J=32.5 Hz, 2H), 1.16-0.97 (m, 2H), 0.85 (d, J=6.8 Hz, 6H). Mass (m/z): 332.3 [M+H]⁺

### Compound 362

### (3aR, 6aS)-N2-(2-(4-isopropylpiperidin-1-yl)pyrimidin-5-yl) octahydropentalene-2,5-diamine

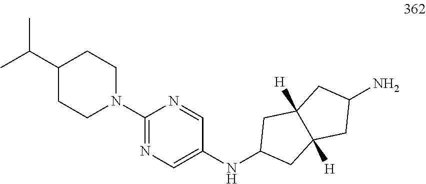 [SMILES: CC(C)C1CCN(c2ncc(NC3C[C@H]4CC(N)C[C@H]4C3)cn2)CC1]

The title compound 362 (16.7 mg) was prepared in a three-step overall yield of 21.8% as a white powder with 1:0.8 mixture by ¹H NMR from 2-(4-isopropylpiperidin-1-yl)pyrimidin-5-amine (106 mg, 0.48 mmol) and (3as,6as)-tetrahydropentalene-2,5 (1H,3H)-dione (200 mg, 1.45 mmol) according to the procedure for 20. ¹H NMR (400 MHZ, DMSO-d₆) δ 8.10 (s, 3H), 7.91 (s, 2H), 5.12 (d, J=5.9 Hz, 1H), 4.48 (d, J=12.8 Hz, 2H), 2.62 (t, J=12.2 Hz, 1H), 2.38 (s, 4H), 2.21 (td, J=15.6, 14.1, 6.9 Hz, 5H), 1.64 (d, J=12.7 Hz, 2H), 1.40 (dq, J=13.7, 8.2, 6.6 Hz, 4H), 1.09 (td, J=12.2, 4.0 Hz, 2H), 0.85 (d, J=6.7 Hz, 6H). Mass (m/z): 344.4 [M+H]⁺.

### Compound 363

### (3aR, 6aS)-N2-(4-(4-(trifluoromethyl)piperidin-1-yl)phenyl) octahydropentalene-2,5-diamine

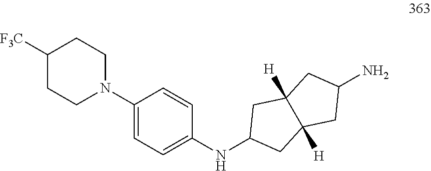 [SMILES: NC1C[C@@H]2CC(Nc3ccc(N4CCC(C(F)(F)F)CC4)cc3)C[C@@H]2C1]

The title compound 363 (30.8 mg) was prepared in a three-step overall yield of 17.5% as a white from powder with 1:0.8 mixture by ¹H NMR 4-(4-(trifluoromethyl)piperidin-1-yl)aniline (118 mg, 0.48 mmol) and (3as,6as)-tetrahydropentalene-2,5 (1H,3H)-dione (200 mg, 1.45 mmol) according to the procedure for 20. ¹H NMR (400 MHZ, DMSO-d₆) δ 8.08 (s, 3H), 6.76 (dd, J=8.7, 6.5 Hz, 2H), 6.50 (dd, J=8.6, 6.2 Hz, 2H), 5.17 (d, J=40.9 Hz, 1H), 3.84-3.50 (m, 1H), 3.42 (d, J=12.3 Hz, 2H), 2.38 (s, 2H), 2.22 (td, J=13.8, 12.2, 6.4 Hz, 3H), 1.86 (d, J=11.9 Hz, 2H), 1.74 (tt, J=16.3, 7.1 Hz, 1H), 1.63-1.50 (m, 2H), 1.50-1.34 (m, 2H), 1.35-1.14 (m, 3H). Mass (m/z): 368.7 [M+H]⁺.

### Compound 364

### N2-(2-fluoro-4-(4-(trifluoromethyl)piperidin-1-yl)phenyl)spiro[3.3]heptane-2,6-diamine

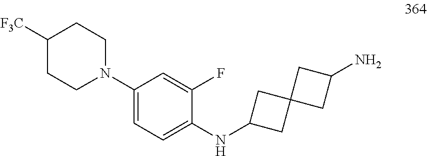 [SMILES: NC1CC2(C1)CC(Nc1ccc(N3CCC(C(F)(F)F)CC3)cc1F)C2]

The title compound 364 (14.5 mg) was prepared in a yield of 11.5% as a white powder with 1:0. 2 mixture by ¹H NMR from 2-fluoro-4-(4-(trifluoromethyl)piperidin-1-yl)aniline (70 mg, 0.27 mmol) and tert-butyl (6-oxospiro [3.3]heptan-2-yl)carbamate (90 mg, 0.40 mmol) according to the procedure for 20. ¹H NMR (400 MHZ, DMSO-d₆) δ 8.15 (s, 3H), 6.73 (dd, J=14.5, 2.6 Hz, 1H), 6.58 (dd, J=8.6, 2.6 Hz, 1H), 6.54-6.42 (m, 1H), 5.01 (d, J=7.1 Hz, 1H), 3.67 (h, J=7.3 Hz, 1H), 3.50 (d, J=12.3 Hz, 3H), 2.56 (dd, J=12.2, 2.5 Hz, 3H), 2.42-2.26 (m, 3H), 2.24-2.07 (m, 3H), 1.96-1.79 (m, 4H), 1.53 (qd, J=12.5, 4.0 Hz, 2H). Mass (m/z): 472.4 [M+H]⁺.

### Compound 365

### N-methyl-4-((4-(4-(trifluoromethyl)piperidin-1-yl)phenyl)amino)cyclohexane-1-carboxamide

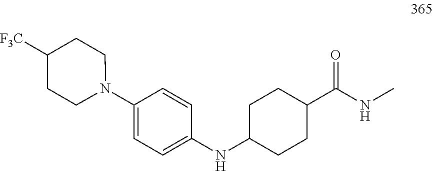 [SMILES: CNC(=O)C1CCC(Nc2ccc(N3CCC(C(F)(F)F)CC3)cc2)CC1]

The title compound 365 (10.1 mg) was prepared in a yield of 9.7% as a white powder from 4-((4-(4-(trifluoromethyl)piperidin-1-yl)phenyl)amino)cyclohexane-1-carboxylic acid (100 mg, 0.27 mmol) and methanamine hydrochloride (73 mg, 1.08 mmol) according to the procedure for 10. ¹H NMR (400 MHZ, DMSO-d₆) δ 7.66 (d, J=4.7 Hz, 1H), 6.79-6.69 (m, 2H), 6.50-6.44 (m, 2H), 4.87 (d, J=8.5 Hz, 1H), 3.41 (d, J=12.0 Hz, 2H), 3.04 (d, J=7.8 Hz, 1H), 2.55 (t, J=4.3 Hz, 5H), 2.42-2.34 (m, 1H), 2.09-1.93 (m, 3H), 1.85 (d, J=12.5 Hz, 2H), 1.78-1.67 (m, 2H), 1.57 (td, J=12.5, 4.0 Hz, 2H), 1.45 (qd, J=13.5, 3.7 Hz, 3H), 1.11-0.97 (m, 2H). Mass (m/z): 384.3 [M+H]⁺.

### Compound 366

### N,N-dimethyl-4-((4-(4-(trifluoromethyl)piperidin-1-yl)phenyl)amino)cyclohexane-1-carboxamide

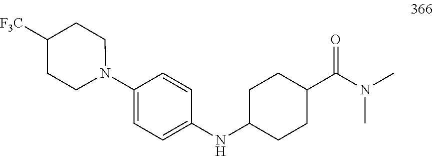 [SMILES: CN(C)C(=O)C1CCC(Nc2ccc(N3CCC(C(F)(F)F)CC3)cc2)CC1]

The title compound 366 (31.9 mg) was prepared in a yield of 29.8% as a white powder from 4-((4-(4-(trifluoromethyl)piperidin-1-yl)phenyl)amino)cyclohexane-1-carboxylic acid (100 mg, 0.27 mmol) and dimethylamine (36 mg, 0.80 mmol) according to the procedure for 10. ¹H NMR (400 MHZ, DMSO-d₆) δ 6.74 (d, J=8.2 Hz, 2H), 6.51 (s, 2H), 4.89 (s, 1H), 3.42 (d, J=11.6 Hz, 2H), 3.06 (s, 1H), 3.00 (s, 3H), 2.79 (s, 3H), 2.04-1.92 (m, 3H), 1.85 (d, J=12.5 Hz, 2H), 1.68 (d, J=13.1 Hz, 2H), 1.56 (d, J=12.4 Hz, 3H), 1.44 (q, J=13.3 Hz, 3H), 1.13 (q, J=12.4 Hz, 3H). Mass (m/z): 398.4 [M+H]⁺.

### Compound 367

### N-((1r,4r)-4-(((2-(4-isopropylpiperidin-1-yl)pyrimidin-5-yl)amino)methyl)cyclohexyl)-5-oxopy rrolidine-3-carboxamide

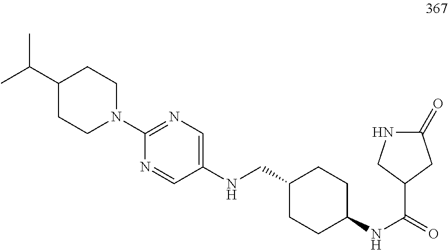 [SMILES: CC(C)C1CCN(c2ncc(NC[C@H]3CC[C@H](NC(=O)C4CNC(=O)C4)CC3)cn2)CC1]

The desired product 367 (32.7 mg, 49.0%) as yellow solid was prepared from N-(((1r,4r)-4-aminocyclohexyl)methyl)-2-(4-isopropylpiperidin-1-yl)pyrimidin-5-amine (50 mg, 0.151 mmol), 5-oxopyrrolidine-3-carboxylic acid (25 mg, 0.196 mmol), HATU (75 mg, 0.196 mmol), DIEA (58 mg, 0.453 mmol) and DMF (5 mL) according to the procedure for 1. ¹H NMR (400 MHZ, DMSO-d₆) δ 8.18 (dt, J=12.8, 5.6 Hz, 1H), 7.94 (s, 1H), 7.88 (s, 1H), 7.59 (s, 1H), 4.91 (dd, J=71.2, 8.4 Hz, 1H), 4.58-4.41 (m, 2H), 3.44-3.38 (m, 2H), 3.25-3.16 (m, 2H), 2.96 (dt, J=30.0, 6.4 Hz, 3H), 2.61 (td, J=12.5, 2.4 Hz, 2H), 2.27 (dd, J=8.4, 4.0 Hz, 2H), 1.94 (d, J=11.2 Hz, 1H), 1.74-1.55 (m, 4H), 1.54-1.45 (m, 2H), 1.44-1.36 (m, 3H), 1.14-0.96 (m, 4H), 0.85 (d, J=6.8 Hz, 6H). Mass (m/z): 443.3 [M+H]⁺.

### Compound 368

### N4-(2-(4-isopropylpiperidin-1-yl)pyrimidin-5-yl)adamantane-1,4-diamine

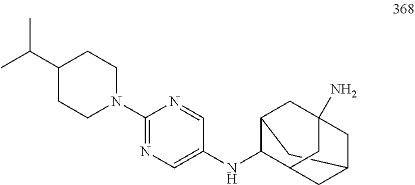 [SMILES: CC(C)C1CCN(c2ncc(NC3C4CC5CC3CC(N)(C5)C4)cn2)CC1]

The title compound 368 (4.6 mg) was prepared in a yield of 2.16% as a white powder with 1:0. 3 mixture by ¹H NMR from 2-(4-isopropylpiperidin-1-yl)pyrimidin-5-amine (100 mg, 0.45 mmol) and tert-butyl (4-oxoadamantan-1-yl)carbamate (180 mg, 0.34 mmol) according to the procedure for 20. ¹H NMR (301 MHz, Methanol-d₄) δ 7.97 (s, 1H), 7.95 (s, 1H), 6.80 (s, 3H), 4.99 (d, J=6.9 Hz, 2H), 2.73 (s, 2H), 2.60 (s, 3H), 2.28-2.10 (m, 5H), 1.98 (d, J=28.3 Hz, 5H), 1.73 (d, J=12.5 Hz, 4H), 1.60 (s, 3H), 0.90 (d, J=3.0 Hz, 6H). Mass (m/z): 470.4 [M+H]⁺.

### Compound 369

### N2-(4-(tert-pentyl)phenyl)spiro[3.3]heptane-2,6-diamine

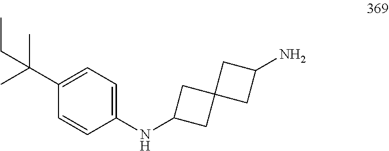 [SMILES: CCC(C)(C)c1ccc(NC2CC3(CC(N)C3)C2)cc1]

The title compound 369 (44.3 mg) was prepared in a yield of 38.5% as a white powder from 4-(tert-pentyl)aniline (100 mg, 0.43 mmol) and tert-butyl (6-oxospiro [3.3]heptan-2-yl)carbamate (116 mg, 0.50 mmol) according to the procedure for 20. 1¹H NMR (400 MHZ, DMSO-d₆) δ 7.03-6.95 (m, 2H), 6.44-6.33 (m, 2H), 5.50 (d, J=6.7 Hz, 1H), 3.63 (h, J=7.3 Hz, 1H), 3.22-3.10 (m, 1H), 2.39 (ddd, J=11.3, 7.1, 4.7 Hz, 1H), 2.35-2.21 (m, 2H), 2.11 (ddd, J=11.8, 7.1, 5.2 Hz, 1H), 1.75 (td, J=11.0, 7.8 Hz, 2H), 1.65 (ddd, J=18.9, 10.8, 8.4 Hz, 2H), 1.51 (q, J=7.4 Hz, 2H), 1.14 (s, 6H), 0.59 (t, J=7.3 Hz, 3H). Mass (m/z): 273.1 [M+H]⁺.

### Compound 370

### N-(5-(aminomethyl) adamantan-2-yl)-2-(4-isopropylpiperidin-1-yl)pyrimidin-5-amine

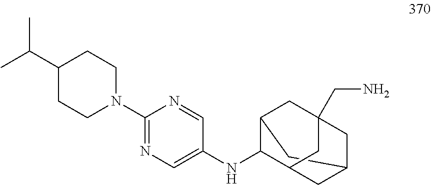 [SMILES: CC(C)C1CCN(c2ncc(NC3C4CC5CC3CC(CN)(C5)C4)cn2)CC1]

The title compound 370 (8.6 mg) was prepared in a two-step overall yield of 6.8% as a white powder from 2-(4-isopropylpiperidin-1-yl)pyrimidin-5-amine (200 mg, 0.90 mmol) and 4-oxoadamantane-1-carboxylic acid (260 mg, 1.36 mmol) according to the procedure for 84. 1¹H NMR (400 MHZ, DMSO-d₆) δ 7.93 (d, J=2.3 Hz, 1H), 7.19 (s, 1H), 6.65 (s, 1H), 5.31 (t, J=4.8 Hz, 1H), 4.46 (d, J=12.2 Hz, 1H), 1.99 (q, J=6.8, 6.2 Hz, 7H), 1.66 (s, 5H), 1.42 (d, J=18.3 Hz, 6H), 1.06-1.01 (m, 3H), 0.84 (dd, J=6.9, 3.8 Hz, 8H). Mass (m/z): 399.8 [M+H]⁺.

### Compound 371

### N-(((1r,4r)-4-aminocyclohexyl)methyl)-2-methyl-6-(4-(trifluoromethyl)piperidin-1-yl)pyridin-3-amine

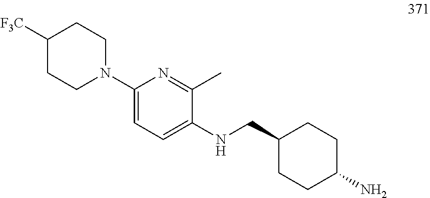 [SMILES: Cc1nc(N2CCC(C(F)(F)F)CC2)ccc1NC[C@H]1CC[C@H](N)CC1]

The title compound 371 (23.6 mg) was prepared in a yield of 23.6% as a white powder from 2-methyl-6-(4-(trifluoromethyl)piperidin-1-yl)pyridin-3-amine (70 mg, 0.27 mmol) and tert-butyl ((1r,4r)-4-formylcyclohexyl)carbamate (114 mg, 0.54 mmol) according to the procedure for 20. ¹H NMR (400 MHZ, DMSO-d₆) δ 7.46 (s, 1H), 7.33 (s, 1H), 7.20 (s, 1H), 6.90 (s, 1H), 6.61 (s, 1H), 4.10 (d, J=12.7 Hz, 2H), 3.03-2.77 (m, 3H), 2.64 (s, 2H), 2.25 (s, 3H), 2.05-1.92 (m, 2H), 1.86 (d, J=15.5 Hz, 4H), 1.48 (s, 3H), 1.36-1.26 (m, 2H), 1.10-0.91 (m, 2H). Mass (m/z): 371.3 [M+H]⁺.

### Compound 372

### (3aR, 6aS)-N2-(2-fluoro-4-(4-(trifluoromethyl)piperidin-1-yl)phenyl)octahydropentalene-2,5-di amine

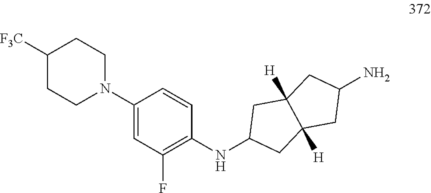 [SMILES: NC1C[C@@H]2CC(Nc3ccc(N4CCC(C(F)(F)F)CC4)cc3F)C[C@@H]2C1]

The title compound 372 (11.2 mg) was prepared in a three-step overall yield of 8.1% as a white powder with 1:0.8 mixture by ¹H NMR from 2-fluoro-4-(4-(trifluoromethyl)piperidin-1-yl)aniline (150 mg, 0.57 mmol) and (3as,6as)-tetrahydropentalene-2,5 (1H,3H)-dione (237 mg, 1.72 mmol) according to the procedure for 20. ¹H NMR (400 MHZ, Methanol-d₄) δ 6.87-6.63 (m, 3H), 3.81 (d, J=11.7 Hz, 1H), 3.52 (ddt, J=15.2, 6.6, 3.6 Hz, 2H), 2.75-2.50 (m, 4H), 2.46-2.32 (m, 3H), 2.25 (dtt, J=17.2, 8.7, 4.5 Hz, 1H), 2.03-1.87 (m, 3H), 1.70 (qd, J=12.6, 4.1 Hz, 3H), 1.48 (td, J=11.8, 7.9 Hz, 1H), 1.39-1.26 (m, 2H). Mass (m/z): 386.2 [M+H]⁺.

### Compound 373

### (3aR,6aS)-N2-ethyl-N5-(2-fluoro-4-(4-(trifluoromethyl)piperidin-1-yl)phenyl)octahydropentale ne-2,5-diamine

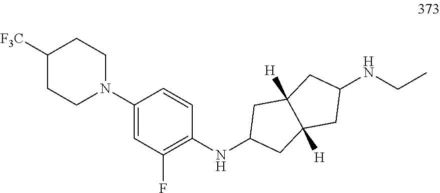 [SMILES: CCNC1C[C@@H]2CC(Nc3ccc(N4CCC(C(F)(F)F)CC4)cc3F)C[C@@H]2C1]

The title compound 373 (7.8 mg) was prepared in a three-step overall yield of 6.5% as a white powder with 1:0.8 mixture by ¹H NMR from 2-fluoro-4-(4-(trifluoromethyl)piperidin-1-yl)aniline (150 mg, 0.57 mmol) and (3as, 6as)-tetrahydropentalene-2,5 (1H,3H)-dione (237 mg, 1.72 mmol) according to the procedure for 20. ¹H NMR (400 MHZ, DMSO-d₆) δ 7.90 (s, 1H), 6.84-6.49 (m, 3H), 3.76 (d, J=12.5 Hz, 1H), 3.50 (t, J=9.9 Hz, 2H), 2.90 (d, J=7.3 Hz, 1H), 2.70-2.62 (m, 3H), 2.26 (d, J=8.5 Hz, 2H), 1.99 (q, J=6.9, 6.4 Hz, 2H), 1.88 (d, J=17.5 Hz, 2H), 1.64-1.38 (m, 4H), 1.23-1.13 (m, 4H), 0.95 (s, 1H), 0.87-0.80 (m, 1H). Mass (m/z): 414.5 [M+H].

### Compound 374

### 2-oxo-N-(6-((4-(4-(trifluoromethyl)piperidin-1-yl)phenyl)amino)spiro[3.3]heptan-2-yl)imidazo lidine-4-carboxamide

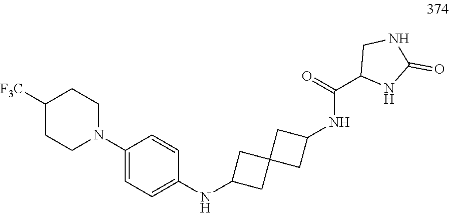 [SMILES: O=C1NCC(C(=O)NC2CC3(C2)CC(Nc2ccc(N4CCC(C(F)(F)F)CC4)cc2)C3)N1]

The title compound 374 (33.4 mg) was prepared in a yield of 50.7% as a white powder from N2-(4-(4-(trifluoromethyl)piperidin-1-yl)phenyl)spiro[3.3]heptane-2,6-diamine (50 mg, 0.14 mmol) and 2-oxoimidazolidine-4-carboxylic acid (22 mg, 0.17 mmol) according to the procedure for 1. ¹H NMR (400 MHZ, DMSO-d₆) δ 8.05 (d, J=7.7 Hz, 1H), 6.78-6.70 (m, 2H), 6.41 (dd, J=8.8, 6.7 Hz, 3H), 6.27 (s, 1H), 5.31 (d, J=6.8 Hz, 1H), 4.10 (h, J=8.0 Hz, 1H), 3.98 (ddd, J=9.7, 6.1, 1.6 Hz, 1H), 3.62 (h, J=7.4 Hz, 1H), 3.49 (t, J=9.3 Hz, 1H), 3.41 (d, J=12.1 Hz, 2H), 3.16 (ddd, J=9.0, 6.1, 1.3 Hz, 1H), 2.40-2.25 (m, 4H), 2.16 (q, J=5.5 Hz, 1H), 1.96 (ddd, J=19.9, 12.8, 9.1 Hz, 3H), 1.90-1.71 (m, 5H), 1.55 (qd, J=12.4, 4.1 Hz, 2H). Mass (m/z): 466.5 [M+H]⁺.

### Compound 375

### 5-oxo-N-(6-(4-(4-(trifluoromethyl)piperidin-1-yl)phenyl)amino)spiro[3.3]heptan-2-yl)pyrrolid ine-3-carboxamide

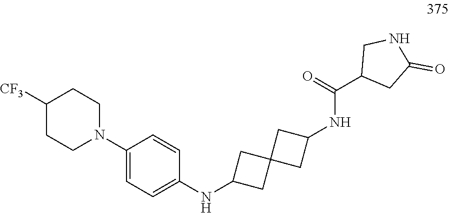 [SMILES: O=C1CC(C(=O)NC2CC3(C2)CC(Nc2ccc(N4CCC(C(F)(F)F)CC4)cc2)C3)CN1]

The title compound 375 (45.1 mg) was prepared in a yield of 68.6% as a white powder from N2-(4-(4-(trifluoromethyl)piperidin-1-yl)phenyl)spiro[3.3]heptane-2,6-diamine (50 mg, 0.14 mmol) and 5-oxopyrrolidine-3-carboxylic acid (22 mg, 0.17 mmol) according to the procedure for 1. ¹H NMR (400 MHZ, DMSO-d₆) δ 8.15 (d, J=7.5 Hz, 1H), 7.54 (s, 1H), 6.80-6.69 (m, 2H), 6.46-6.34 (m, 2H), 5.31 (d, J=6.8 Hz, 1H), 4.06 (h, J=8.0 Hz, 1H), 3.62 (h, J=7.4 Hz, 1H), 3.40 (d, J=12.4 Hz, 2H), 3.36 (dd, J=8.9, 1.9 Hz, 1H), 3.16 (ddd, J=9.6, 6.5, 3.5 Hz, 1H), 3.04 (qd, J=8.6, 6.5 Hz, 1H), 2.54 (d, J=2.5 Hz, 2H), 2.39-2.27 (m, 3H), 2.27-2.20 (m, 2H), 2.16 (dt, J=11.9, 6.2 Hz, 1H), 2.03-1.69 (m, 7H), 1.55 (qd, J=12.5, 4.1 Hz, 2H). Mass (m/z): 465.7 [M+H]⁺.

### Compound 376

### N2-(2-methyl-6-(4-(trifluoromethyl)piperidin-1-yl)pyridin-3-yl) spiro[3.3]heptane-2,6-diamine

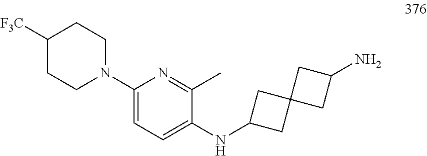 [SMILES: Cc1nc(N2CCC(C(F)(F)F)CC2)ccc1NC1CC2(CC(N)C2)C1]

The title compound 376 (19.3 mg) was prepared in a yield of 19.4% as a white powder from 2-methyl-6-(4-(trifluoromethyl)piperidin-1-yl)pyridin-3-amine (70 mg, 0.27 mmol) and tert-butyl (6-oxospiro [3.3]heptan-2-yl)carbamate (79 mg, 0.35 mmol) according to the procedure for 20. ¹H NMR (400 MHZ, Methanol-d₄) δ 6.91 (d, J=9.5 Hz, 1H), 6.62 (s, 1H), 4.04 (s, 2H), 3.69 (p, J=8.1 Hz, 2H), 2.83-2.52 (m, 4H), 2.47 (dt, J=11.7, 6.0 Hz, 1H), 2.41-2.10 (m, 7H), 2.10-1.79 (m, 4H), 1.72-1.55 (m, 2H). Mass (m/z): 369.7 [M+H]⁺.

### Compound 377

### N-((4-((2-methyl-6-(4-(trifluoromethyl)piperidin-1-yl)pyridin-3-yl)amino)cyclohexyl)methyl)-5-oxopyrrolidine-3-carboxamide

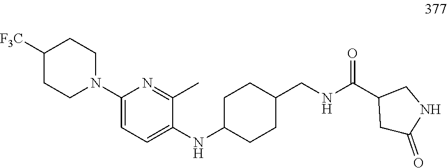 [SMILES: Cc1nc(N2CCC(C(F)(F)F)CC2)ccc1NC1CCC(CNC(=O)C2CNC(=O)C2)CC1]

The title compound 377 (10.6 mg) was prepared in a yield of 16.3% as a white powder from N-(4-(aminomethyl)cyclohexyl)-2-methyl-6-(4-(trifluoromethyl)piperidin-1-yl)pyridin-3-amin e (50 mg, 0.13 mmol) and 5-oxopyrrolidine-3-carboxylic acid (21 mg, 0.16 mmol) according to the procedure for 10. ¹H NMR (400 MHz, Methanol-d₄) δ 7.05 (s, 1H), 4.06 (s, 2H), 3.58 (dd, J=9.9, 8.8 Hz, 2H), 3.46 (dd, J=9.9, 6.4 Hz, 2H), 3.35 (s, 1H), 3.18 (d, J=7.1 Hz, 2H), 3.08 (dd, J=6.8, 1.2 Hz, 1H), 2.60-2.43 (m, 4H), 1.93 (s, 3H), 1.80-1.51 (m, 6H), 1.45-1.35 (m, 2H), 1.16-1.01 (m, 1H). Mass (m/z): 482.4 [M+H]⁺.

### Compound 378

### N-((1r,4r)-4-(((2-methyl-6-(4-(trifluoromethyl)piperidin-1-yl)pyridin-3-yl)amino)methyl)cycloh exyl)-5-oxopyrrolidine-3-carboxamide

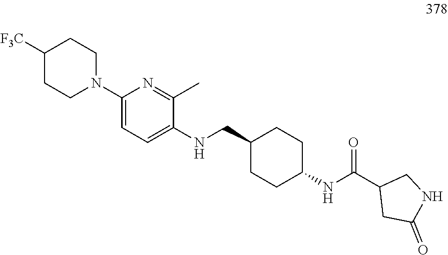 [SMILES: Cc1nc(N2CCC(C(F)(F)F)CC2)ccc1NC[C@H]1CC[C@H](NC(=O)C2CNC(=O)C2)CC1]

The title compound 378 (12.0 mg) was prepared in a yield of 16.3% as a white powder from N-(((1r,4r)-4-aminocyclohexyl)methyl)-2-methyl-6-(4-(trifluoromethyl)piperidin-1-yl)pyridin-3-amine (40 mg, 0.11 mmol) and 5-oxopyrrolidine-3-carboxylic acid (17 mg, 0.13 mmol) according to the procedure for 10. ¹H NMR (400 MHZ, DMSO-d₆) δ 7.86 (d, J=7.9 Hz, 1H), 7.56 (s, 1H), 6.83 (d, J=8.8 Hz, 1H), 6.55 (d, J=8.7 Hz, 1H), 4.41 (t, J=6.0 Hz, 1H), 4.11 (d, J=12.7 Hz, 2H), 3.50 (tt, J=7.4, 3.8 Hz, 1H), 3.41-3.34 (m, 1H), 3.19 (dd, J=9.3, 6.4 Hz, 1H), 3.12-3.00 (m, 1H), 2.84 (t, J=6.3 Hz, 2H), 2.60 (td, J=12.5, 2.5 Hz, 3H), 2.38-2.23 (m, 3H), 2.22 (s, 3H), 1.88-1.74 (m, 6H), 1.46 (qd, J=12.5, 4.4 Hz, 3H), 1.20-1.05 (m, 2H), 1.05-0.92 (m, 2H). Mass (m/z): 482.2 [M+H]⁺.

### Compound 379

### N-(((1r,4r)-4-aminocyclohexyl)methyl)-2-methyl-4-(piperidin-1-yl)aniline

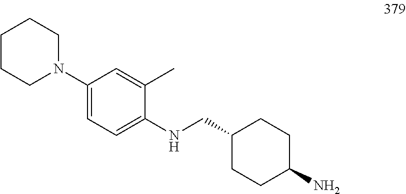 [SMILES: Cc1cc(N2CCCCC2)ccc1NC[C@H]1CC[C@H](N)CC1]

The title compound 379 (21 mg) was prepared in a yield of 49.5% as a white powder from 2-methyl-4-(piperidin-1-yl)aniline (40 mg, 0.23 mmol) and tert-butyl ((1r,4r)-4-formylcyclohexyl)carbamate (54 mg. 0.33 mmol) according to the procedure for 20. 1¹H NMR (400 MHZ, DMSO-d₆) δ 8.01 (s, 3H), 6.67 (s, 2H), 6.38 (s, 1H), 4.37 (s, 1H), 2.88 (d, J=25.2 Hz, 5H), 2.04 (s, 3H), 1.96 (t, J=8.3 Hz, 2H), 1.85 (d, J=12.8 Hz, 2H), 1.54 (d, J=58.9 Hz, 7H), 1.35-1.20 (m, 3H), 0.98 (q, J=12.7 Hz, 2H). Mass (m/z): 302.6 [M+H]⁺.

### Compound 380

### N2-(2-methyl-4-(piperidin-1-yl)phenyl)spiro[3.3]heptane-2,6-diamine

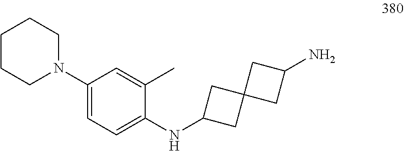 [SMILES: Cc1cc(N2CCCCC2)ccc1NC1CC2(CC(N)C2)C1]

The title compound 380 (26 mg) was prepared in a yield of 59.2% as a white powder from 2-methyl-4-(piperidin-1-yl)aniline (40 mmol) and mg, 0.2 tert-butyl (6-oxospiro [3.3]heptan-2-yl)carbamate (51 mg, 0.32 mmol) according to the procedure for 20. ¹H NMR (400 MHZ, DMSO-d₆) δ 8.30-8.05 (m, 3H), 7.51-7.07 (m, 1H), 6.38 (s, 1H), 3.71 (s, 1H), 3.55 (s, 1H), 3.24-2.93 (m, 2H), 2.39 (dd, J=12.4, 6.7 Hz, 2H), 2.17 (q, J=10.4, 9.6 Hz, 3H), 2.06 (s, 3H), 2.02-1.87 (m, 3H), 1.74 (s, 3H), 1.48 (d, J=30.5 Hz, 2H), 1.32-1.17 (m, 3H). Mass (m/z): 300.6 [M+H]⁺.

### Compound 381

### N1-(4-(4,4-dimethylpiperidin-1-yl)-2-methylphenyl)cyclohexane-1,4-diamine

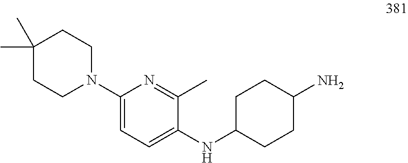 [SMILES: Cc1nc(N2CCC(C)(C)CC2)ccc1NC1CCC(N)CC1]

The title compound 381 (22 mg) was prepared in a yield of 43.2% as a white powder from 4-(4,4-dimethylpiperidin-1-yl)-2-methylaniline (40 mg, 0.18 mmol) and tert-butyl (4-oxocyclohexyl)carbamate (45 mg, 0.24 mmol) according to the procedure for 20. ¹H NMR (400 MHZ, DMSO-d₆) δ 7.96 (s, 3H), 6.55 (s, 2H), 3.04 (d, J=63.1 Hz, 5H), 2.23-1.88 (m, 6H), 1.75 (d, J=41.8 Hz, 4H), 1.45 (s, 5H), 0.96 (d, J=4.2 Hz, 6H). Mass (m/z): 316.4 [M+H]⁺.

### Compound 382

### N-(((1r,4r)-4-aminocyclohexyl)methyl)-4-(4,4-dimethylpiperidin-1-yl)-2-methylaniline

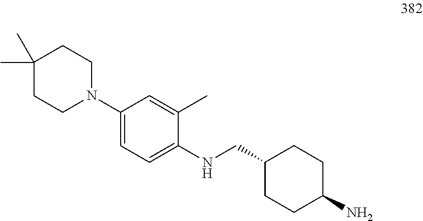 [SMILES: Cc1cc(N2CCC(C)(C)CC2)ccc1NC[C@H]1CC[C@H](N)CC1]

The title compound 382 (38 mg) was prepared in a yield of 64.2% as a white powder from 4-(4,4-dimethylpiperidin-1-yl)-2-methylaniline (55 mg, 0.23 mmol) and tert-butyl ((1r,4r)-4-formylcyclohexyl)carbamate (64 mg. 0.31 mmol) according to the procedure for 20. 1¹H NMR (400 MHZ, DMSO-d₆) δ 7.96 (s, 3H), 7.38-7.02 (m, 1H), 6.61 (d, J=48.8 Hz, 2H), 3.04 (d, J=63.1 Hz, 5H), 2.04 (dd, J=43.5, 19.9 Hz, 6H), 1.75 (d, J=41.8 Hz, 4H), 1.56-1.35 (m, 5H), 1.28 (s, 1H), 0.96 (d, J=4.2 Hz, 6H). Mass (m/z): 330.5 [M+H]⁺.

### Compound 383

### N-(2-((1r,4r)-4-aminocyclohexyl)ethyl)-2-methyl-4-(4-(trifluoromethyl)piperidin-1-yl)aniline

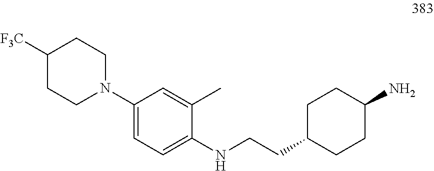 [SMILES: Cc1cc(N2CCC(C(F)(F)F)CC2)ccc1NCC[C@H]1CC[C@H](N)CC1]

The title compound 383 (18 mg) was prepared in a yield of 23.5% as a white powder from 2-methyl-4-(4-(trifluoromethyl)piperidin-1-yl)aniline (70 mg, 0.32 mmol) and tert-butyl ((1r,4r)-4-(2-oxoethyl)cyclohexyl)carbamate (95 mg, 0.46 mmol) according to the procedure for 20. ¹H NMR (400 MHZ, DMSO-d₆) δ 7.88 (s, 3H), 6.70 (s, 1H), 6.65 (d, J=8.8 Hz, 1H), 6.42 (d, J=8.6 Hz, 1H), 4.27 (s, 1H), 3.46 (t, J=12.0 Hz, 2H), 3.01 (s, 2H), 2.93 (s, 1H), 2.37 (d, J=10.7 Hz, 1H), 2.04 (s, 3H), 1.93 (d, J=12.3 Hz, 3H), 1.83 (t, J=16.9 Hz, 4H), 1.56 (d, J=12.1 Hz, 2H), 1.47 (d, J=7.0 Hz, 2H), 1.36-1.25 (m, 3H), 1.07-0.92 (m, 2H). Mass (m/z): 384.3 [M+H]⁺.

### Compound 384

### N1-(3-(4-(trifluoromethyl)piperidin-1-yl)phenyl)cyclohexane-1,4-diamine

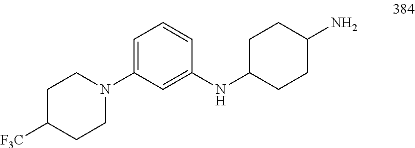 [SMILES: NC1CCC(Nc2cccc(N3CCC(C(F)(F)F)CC3)c2)CC1]

The title compound 384 (64 mg, 55.8%) as a yellow solid was prepared from tert-butyl (4-((3-(4-(trifluoromethyl)piperidin-1-yl)phenyl)amino)cyclohexyl)carbamate according to the procedure for 24. ¹H NMR (400 MHZ, DMSO-d₆) δ 7.90 (d, J=12.0, 1H), 7.13-7.02 (m, 1H), 6.59-6.32 (m, 2H), 3.81-3.62 (m, 2H), 3.46 (s, 1H), 3.25 (s, 1H), 3.19-3.08 (m, 1H), 2.95 (d, J=4.0, 2H), 2.63-2.54 (m, 2H), 2.05-1.85 (m, 4H), 1.85-1.51 (m, 6H), 1.33 (m, 2H). Mass (m/z): 341.9 [M+H]⁺.

### Compound 385

### N1-(((1r,4r)-4-aminocyclohexyl)methyl)-N4-ethyl-N4-(3-methoxypropyl) benzene-1,4-diamine

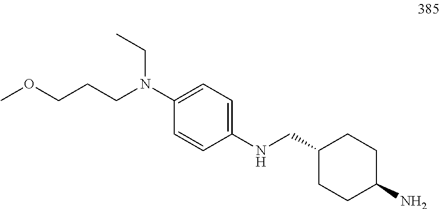 [SMILES: CCN(CCCOC)c1ccc(NC[C@H]2CC[C@H](N)CC2)cc1]

The title compound 385 (24.8 mg) was prepared in a total yield of 47.9% as a purple solid from tert-butyl ((1r,4r)-4-(((4-(ethyl (3-methoxypropyl)amino)phenyl)amino)methyl)cyclohexyl)carbamate (68 mg, 0.162 mmol), TFA (1 mL) and DCM (10 mL) according to the procedure for 24. ¹H NMR (400 MHZ, DMSO-d₆) δ 6.88-6.39 (m, 4H), 3.23-3.20 (m, 3H), 3.18 (d, J=2.4 Hz, 2H), 2.82 (q, J=7.2 Hz, 3H), 2.00-1.90 (m, 3H), 1.86-1.77 (m, 3H), 1.69-1.59 (m, 4H), 1.30 (t, J=12.0 Hz, 2H), 1.16 (t, J=7.2 Hz, 2H), 1.08-0.93 (m, 7H). Mass (m/z): 320.3 [M+H]⁺.

### Compound 386

### N1-(((1r,4r)-4-aminocyclohexyl)methyl)-N4-ethyl-N4-pentylbenzene-1,4-diamine

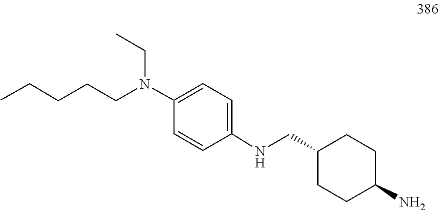 [SMILES: CCCCCN(CC)c1ccc(NC[C@H]2CC[C@H](N)CC2)cc1]

The title compound 386 (41.8 mg) was prepared in a total yield of 71.5% as a purple solid from tert-butyl ((1r,4r)-4-(((4-(ethyl (pentyl)amino)phenyl)amino)methyl)cyclohexyl)carbamate (77 mg, 0.185 mmol), TFA (1 mL), and DCM (10 mL) according to the procedure for 1-2. ¹H NMR (400 MHZ, DMSO-d₆) δ 6.70-6.47 (m, 4H), 3.25-3.17 (m, 3H), 2.92-2.73 (m, 4H), 1.94 (d, J=12.4 Hz, 3H), 1.83 (d, J=12.8 Hz, 2H), 1.49-1.38 (m, 4H), 1.28-1.25 (m, 3H), 0.97-0.91 (m, 4H), 0.86-0.75 (m, 8H). Mass (m/z): 318.3 [M+H]⁺.

### Compound 387

### N-(((1r,4r)-4-aminocyclohexyl)methyl)-4-(2,6-dimethylmorpholino)-2-methylaniline

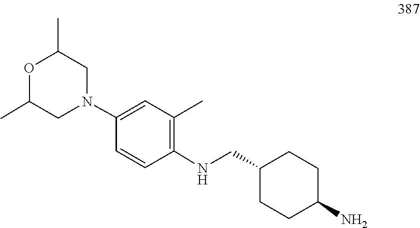 [SMILES: Cc1cc(N2CC(C)OC(C)C2)ccc1NC[C@H]1CC[C@H](N)CC1]

The title compound 387 (38.5 mg) was prepared in a total yield of 57.0% as a purple solid from tert-butyl ((1r,4r)-4-(((4-(2,6-dimethylmorpholino)-2-methylphenyl)amino)methyl)cyclohexyl)carbamate (88 mg, 0.204 mmol), TFA (1 mL), and DCM (10 mL) according to the procedure for 24. ¹H NMR (400 MHZ, DMSO-d₆) δ 8.23 (s, 3H), 6.63 (d, J=2.8 Hz, 1H), 6.57 (dd, J=8.8, 2.8 Hz, 1H), 6.35 (d, J=8.8 Hz, 1H), 4.33 (s, 1H), 3.62 (dqd, J=12.4, 6.0, 2.0 Hz, 2H), 3.23 (dt, J=10.8, 2.0 Hz, 2H), 2.85 (s, 1H), 2.83-2.77 (m, 2H), 2.09-2.04 (m, 2H), 2.01 (s, 3H), 1.96 (dt, J=13.2, 3.2 Hz, 2H), 1.82 (dd, J=13.6, 3.6 Hz, 2H), 1.55-1.43 (m, 1H), 1.30 (qd, J=12.4, 3.2 Hz, 2H), 1.07 (d, J=6.0 Hz, 6H), 1.00-0.88 (m, 2H). Mass (m/z): 332.3 [M+H]⁺.

### Compound 388

### 4-((2-(4-isopropylpiperidin-1-yl)pyrimidin-5-yl)amino)adamantane-1-carboxamide

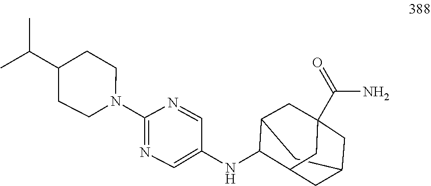 [SMILES: CC(C)C1CCN(c2ncc(NC3C4CC5CC3CC(C(N)=O)(C5)C4)cn2)CC1]

The title compound 388 (34 mg) was prepared in a yield of 47.8% as a white powder from 2-(4-isopropylpiperidin-1-yl)pyrimidin-5-amine (70 mg, 0.27 mmol) and 4-oxoadamantane-1-carboxamide (73 mg, 0.34 mmol) according to the procedure for 20. H NMR (400 MHZ, DMSO-d₆) δ 7.94 (d, J=4.0 Hz, 2H), 6.97 (s, 1H), 6.72 (s, 1H), 5.00 (dd, J=8.2, 4.6 Hz, 1H), 4.47 (dt, J=12.9, 2.8 Hz, 2H), 2.61 (td, J=12.8, 2.4 Hz, 2H), 2.03-1.89 (m, 4H), 1.89-1.79 (m, 4H), 1.72 (dd, J=12.1, 4.0 Hz, 3H), 1.67-1.60 (m, 2H), 1.43-1.33 (m, 3H), 1.18 (ddd, J=12.1, 6.0, 3.0 Hz, 1H), 1.07 (qd, J=12.3, 4.0 Hz, 2H), 0.85 (d, J=6.7 Hz, 6H). Mass (m/z): 398.4 [M+H]⁺.

### Compound 389

### N-(((1r,4r)-4-aminocyclohexyl)methyl)-4-(4-ethylpiperidin-1-yl)-2-methylaniline

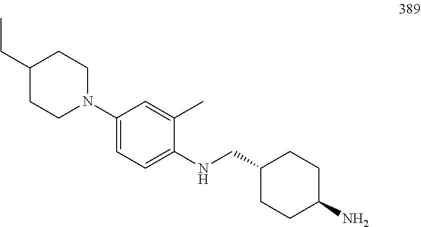 [SMILES: CCC1CCN(c2ccc(NC[C@H]3CC[C@H](N)CC3)c(C)c2)CC1]

The title compound 389 (18 mg) was prepared in a yield of 28.4% as a pale rosy powder from 4-(4-ethylpiperidin-1-yl)-2-methylaniline (70 mg, 0.23 mmol) and tert-butyl ((1r,4r)-4-formylcyclohexyl)carbamate (83 mg, 0.32 mmol) according to the procedure for 20. 1¹H NMR (400 MHZ, DMSO-d₆) δ 7.98 (s, 3H), 6.47 (s, 1H), 2.92 (s, 4H), 2.08 (s, 3H), 1.99 (d, J=22.8 Hz, 3H), 1.85 (s, 4H), 1.55 (s, 2H), 1.29 (q, J=14.1, 13.6 Hz, 5H), 1.00 (d, J=13.2 Hz, 2H), 0.90 (t, J=7.3 Hz, 4H). Mass (m/z): 330.3 [M+H]⁺.

### Compound 390

### N1-(4-(4-ethylpiperidin-1-yl)-2-methylphenyl)cyclohexane-1,4-diamine

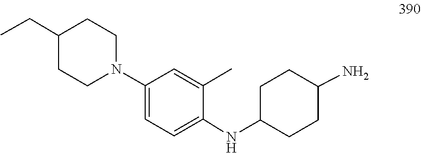 [SMILES: CCC1CCN(c2ccc(NC3CCC(N)CC3)c(C)c2)CC1]

The title compound 390 (29 mg) was prepared in a yield of 54.6% as a pale rosy powder from 4-(4-ethylpiperidin-1-yl)-2-methylaniline 0 mg, 0.23 mmol) and tert-butyl (4-oxocyclohexyl)carbamate (75 mg, 0.32 mmol) according to the procedure for 20. ¹H NMR (400 MHZ, DMSO-d₆) δ 8.04 (s, 3H), 6.65 (s, 2H), 6.48 (s, 1H), 3.70 (s, 1H), 3.48 (s, 1H), 3.36 (d, J=11.3 Hz, 2H), 3.13 (d, J=22.8 Hz, 1H), 2.95 (s, 1H), 2.11 (s, 1H), 2.01 (d, J=19.5 Hz, 3H), 1.75 (d, J=32.9 Hz, 6H), 1.44 (q, J=12.1 Hz, 1H), 1.25 (dd, J=12.3, 5.4 Hz, 6H), 0.88 (t, J=7.2 Hz, 3H). Mass (m/z): 316.4 [M+H]⁺.

### Compound 391

### N-(2-((1r,4r)-4-aminocyclohexyl)ethyl)-2,6-dimethyl-4-(4-(trifluoromethyl)piperidin-1-yl) aniline

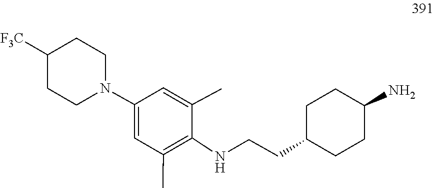 [SMILES: Cc1cc(N2CCC(C(F)(F)F)CC2)cc(C)c1NCC[C@H]1CC[C@H](N)CC1]

The title compound 391 (11 mg) was prepared in a yield of 17.5% as a pale yellow powder from 2,6-dimethyl-4-(4-(trifluoromethyl)piperidin-1-yl)aniline (100 mg, 0.35 mmol) and tert-butyl ((1r,4r)-4-(2-oxoethyl)cyclohexyl)carbamate (83 mg, 0.43 mmol) according to the procedure for 20. ¹H NMR (400 MHZ, DMSO-d₆) δ 7.74 (s, 3H), 6.56 (s, 2H), 4.09 (d, J=5.2 Hz, 1H), 3.58 (d, J=12.4 Hz, 2H), 3.16 (d, J=5.2 Hz, 2H), 2.75 (d, J=6.5 Hz, 2H), 2.16 (s, 6H), 1.95-1.80 (m, 5H), 1.75 (d, J=12.9 Hz, 2H), 1.52 (d, J=9.1 Hz, 3H), 1.37 (s, 2H), 0.96 (d, J=12.6 Hz, 2H). Mass (m/z): 398.5 [M+H]⁺.

### Compound 392

### (3aR,6aS)-N2-(2-methyl-4-(piperidin-1-yl)phenyl) octahydropentalene-2,5-diamine

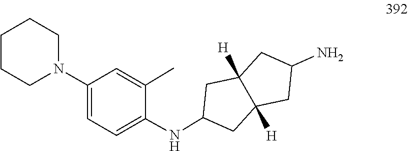 [SMILES: Cc1cc(N2CCCCC2)ccc1NC1C[C@H]2CC(N)C[C@H]2C1]

The title compound 392 (32 mg) was prepared in a three-step total yield of 15.3% as a white solid with 1:1 mixture by ¹H NMR from 2-methyl-4-(piperidin-1-yl)aniline (100 mg, 0.63 mmol) and (3as,6as)-tetrahydropentalene-2,5 (1H,3H)-dione (278 mg, 2.0 mmol) according to the procedure for 20. ¹H NMR (400 MHZ, DMSO-d₆) δ 8.39 (s, 3H), 7.65-7.21 (m, 3H), 6.62 (s, 1H), 3.83 (d, J=68.0 Hz, 2H), 2.39 (s, 2H), 2.31-2.16 (m, 3H), 2.11 (s, 4H), 1.77 (s, 4H), 1.58 (s, 2H), 1.46 (q, J=11.9, 11.1 Hz, 3H), 1.36 (s, 2H). Mass (m/z): 314.6 [M+H]⁺.

### Compound 393

### N¹-(((1r,4r)-4-aminocyclohexyl)methyl)-N4,N4,2-trimethylbenzene-1,4-diamine

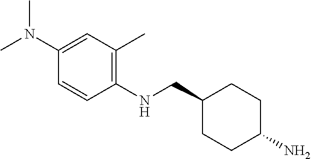 [SMILES: Cc1cc(N(C)C)ccc1NC[C@H]1CC[C@H](N)CC1]

The title compound 393 (15.2 mg) was prepared in a total yield of 50.2% as a Purple solid according to the procedure for compound 24. ¹H NMR (400 MHZ, DMSO-d₆) δ 7.78 (s, 3H), 7.27-7.12 (m, 2H), 6.64-6.48 (m, 1H), 3.60-3.50 (m, 2H), 3.38 (s, 6H), 2.96-2.93 (m, 1H), 2.12 (s, 3H), 1.99-1.85 (m, 4H), 1.63-1.51 (m, 1H), 1.32-1.19 (m, 4H). Mass (m/z): 262.2 [M+H]⁺.

### Compound 394

### N-(2-((1r, 4r)-4-aminocyclohexyl)ethyl)-2-fluoro-4-(4-(trifluoromethyl)piperidin-1-yl)aniline

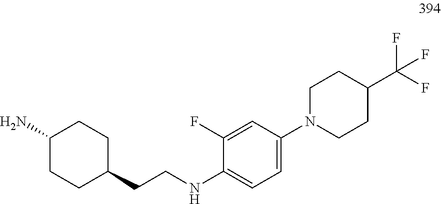 [SMILES: N[C@H]1CC[C@H](CCNc2ccc(N3CCC(C(F)(F)F)CC3)cc2F)CC1]

The desired product 394 as a white solid (19.7 mg, 26.6%) was prepared from tert-butyl ((11,4r)-4-(2-((2-fluoro-4-(4-(trifluoromethyl)piperidin-1-yl)phenyl)amino)ethyl)cyclohexyl)ca rbamate according to the procedure for 24. ¹H NMR (400 MHZ, DMSO-d₆) δ 7.81 (s, 3H), 6.75-6.66 (m, 1H), 6.61-6.50 (m, 2H), 4.73 (s, 1H), 3.54-3.42 (m, 2H), 2.98 (q, J=6.8 Hz, 2H), 2.94-2.79 (m, 1H), 2.41-2.32 (m, 1H), 1.94-1.71 (m, 6H), 1.51 (qd, J=12.5, 4.1 Hz, 2H), 1.40 (q, J=7.0 Hz, 2H), 1.24 (q, J=13.0, 11.9 Hz, 4H), 1.01-0.86 (m, 2H). Mass (m/z): 388.2 [M+H]⁺.

### Compound 395

### N-(((1r, 4r)-4-aminocyclohexyl)methyl)-2-fluoro-6-(4-(trifluoromethyl)piperidin-1-yl)pyridin-3-amine

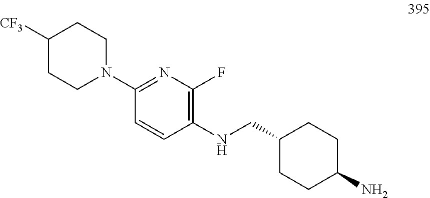 [SMILES: N[C@H]1CC[C@H](CNc2ccc(N3CCC(C(F)(F)F)CC3)nc2F)CC1]

The title compound 395 (9.5 mg, 8.5%) as a yellow solid was prepared from tert-butyl ((1r,4r)-4-(((2-fluoro-6-(4-(trifluoromethyl)piperidin-1-yl)pyridin-3-yl)amino)methyl)cyclohex yl)carbamate (140 mg, 0.3 mmol), DCM (5 mL) and TFA (1 mL) according to the procedure for 24. ¹H NMR (400 MHZ, CD₃OD) δ 7.17-7.01 (m, 1H), 6.50 (d, J=7.6, 1H), 4.04-4.01 (m, 2H), 2.99-2.89 (m, 3H), 2.68-2.61 (m, 1H), 2.29-2.63 (m, 1H), 1.98-1.81 (m, 6H), 1.52-1.44 (m, 3H), 1.34-1.19 (m, 2H), 1.09-0.99 (m, 2H). Mass (m/z): 375.2 [M+H]⁺.

### Compound 396

### N-(((1r,4r)-4-aminocyclohexyl)methyl)-6-(4-methylpiperidin-1-yl)-5-(trifluoromethyl)pyridin-3-amine

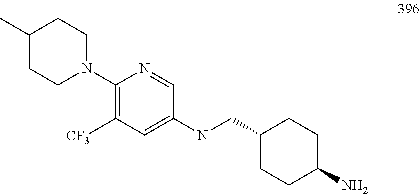

The title compound 396.1 mg, 17.8%) To a solution of tert-butyl ((1r,4r)-4-(((6-(4-methylpiperidin-1-yl)-5-(trifluoromethyl)pyridin-3-yl)amino)methyl)cyclohe xyl)carbamate (200 mg, 0.52 mmol), 1,4-dioxane (2 mL) and HCl/1,4-Dioxane (5 mL) according to the procedure for 37. ¹H NMR (400 MHz, DMSO-d₆) δ 7.95 (d, J=2.8 Hz, 1H), 7.14 (d, J=2.8 Hz, 1H), 6.09 (t, J=5.6 Hz, 1H), 2.91-2.86 (m, 4H), 2.75 (m, 3H), 1.86 (br, 4H), 1.64 (d, J=10.4 Hz, 2H), 1.49-1.41 (m, 2H), 1.26-1.16 (m, 4H), 1.06-0.98 (m, 2H), 0.94 (d, J=6.4 Hz, 3H). Mass (m/z): 371.3 [M+H]⁺.

### Compound 397

### N2-(6-(4-(trifluoromethyl)piperidin-1-yl)pyridin-3-yl)spiro[3.3]heptane-2,6-diamine

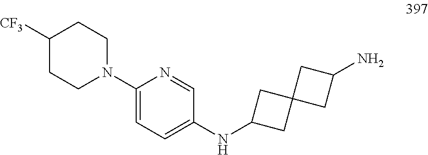 [SMILES: NC1CC2(C1)CC(Nc1ccc(N3CCC(C(F)(F)F)CC3)nc1)C2]

The title compound 397 (42.9 mg) was prepared in a yield of 47.4% as a white powder from 6-(4-(trifluoromethyl)piperidin-1-yl)pyridin-3-amine (70 mg, 0.22 mmol) and tert-butyl (6-oxospiro [3.3]heptan-2-yl)carbamate (77 mg, 0.31 mmol) according to the procedure for 20. ¹H NMR (400 MHZ, DMSO-d₆) δ 8.30-8.14 (m, 3H), 7.46 (d, J=2.9 Hz, 1H), 6.97 (d, J=9.6 Hz, 1H), 6.79 (d, J=9.0 Hz, 1H), 4.11 (d, J=12.8 Hz, 2H), 3.66 (p, J=7.4 Hz, 1H), 3.54 (s, 1H), 3.34 (s, 1H), 2.79-2.65 (m, 2H), 2.47 (dd, J=5.5, 3.2 Hz, 1H), 2.42-2.29 (m, 2H), 2.24-2.13 (m, 3H), 1.83 (q, J=8.2, 7.8 Hz, 4H), 1.45 (qd, J=12.5, 4.1 Hz, 2H). Mass (m/z): 355.2 [M+H]⁺.

### Compound 398

### N-(((1r,4r)-4-aminocyclohexyl)methyl)-6-(4-(trifluoromethyl)piperidin-1-yl)pyridin-3-amine

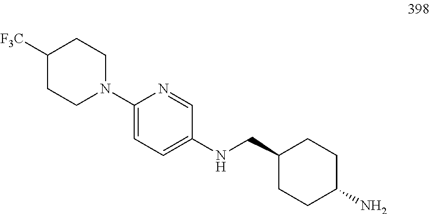 [SMILES: N[C@H]1CC[C@H](CNc2ccc(N3CCC(C(F)(F)F)CC3)nc2)CC1]

The title compound 398 (50 mg) was prepared in a yield of 56.8% as a white powder from 6-(4-(trifluoromethyl)piperidin-1-yl)pyridin-3-amine (80 mg, 0.29 mmol) and tert-butyl ((1r,4r)-4-formylcyclohexyl)carbamate (85 mg, 0.37 mmol) according to the procedure for 20. ¹H NMR (400 MHZ, DMSO-d₆) δ 7.98-7.84 (m, 3H), 7.52 (s, 1H), 7.02 (s, 1H), 6.79 (d, J=9.0 Hz, 1H), 5.31 (s, 1H), 4.08 (d, J=12.8 Hz, 2H), 2.92 (d, J=4.9 Hz, 1H), 2.80 (d, J=6.6 Hz, 2H), 2.69 (t, J=12.1 Hz, 2H), 1.99-1.89 (m, 2H), 1.89-1.76 (m, 4H), 1.44 (qd, J=12.6, 4.2 Hz, 3H), 1.27 (dt, J=14.9, 11.2 Hz, 2H), 1.09-0.90 (m, 2H). Mass (m/z): 357.5 [M+H]⁺.

### Compound 399

### N1-(5-(4-isopropylpiperidin-1-yl)pyrazin-2-yl)cyclohexane-1,4-diamine

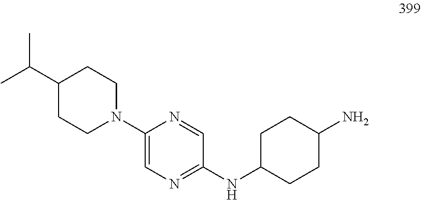 [SMILES: CC(C)C1CCN(c2cnc(NC3CCC(N)CC3)cn2)CC1]

The title compound 399 (29.0 mg) was prepared in a total yield of 29.3% as a yellow solid from tert-butyl (4-((5-(4-isopropylpiperidin-1-yl)pyrazin-2-yl)amino)cyclohexyl)carbamate (130 mg, 0.312 mmol), TFA (1 mL), and DCM (10 mL) according to the procedure for 1-2. H NMR (400 MHZ, DMSO-d₆) δ 7.70-7.54 (m, 2H), 6.11 (dd, J=6.8, 3.2 Hz, 1H), 3.89 (d, J=12.0 Hz, 2H), 2.89 (2, J=13.0 Hz, 1H), 2.50 (d, J=1.6 Hz, 2H), 1.95 (t, J=12.0 Hz, 4H), 1.70 (d, J=6.4 Hz, 2H), 1.64 (d, J=12.0 Hz, 3H), 1.55 (s, 1H), 1.46-1.35 (m, 4H), 1.16-1.11 (m, 3H), 0.83 (s, 3H), 0.82 (s, 3H). Mass (m/z): 318.3 [M+H]⁺.

### Compound 400

### N1-(5-(4-isopropylpiperidin-1-yl)pyrimidin-2-yl)cyclohexane-1,4-diamine

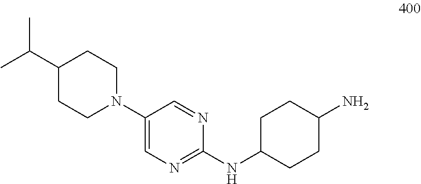 [SMILES: CC(C)C1CCN(c2cnc(NC3CCC(N)CC3)nc2)CC1]

The title compound 400 (11.2 mg) was prepared in a total yield of 30.7% as a white solid from tert-butyl (4-((5-(4-isopropylpiperidin-1-yl)pyrimidin-2-yl)amino)cyclohexyl)carbamate (48 mg, 0.115 mmol), TFA (1 mL), and DCM (10 mL) according to the procedure for 24. H NMR (400 MHZ, DMSO-d₆) δ 8.18 (s, 2H), 8.04 (d, J=8.4 Hz, 2H), 3.36 (d, J=9.6 Hz, 2H), 2.50 (s, 2H), 2.41 (s, 2H), 1.90 (d, J=18.0 Hz, 3H), 1.71-1.49 (m, 4H), 1.40 (dd, J=13.2, 7.2 Hz, 3H), 1.26 (td, J=12.4, 3.6 Hz, 4H), 1.03 (s, 1H), 0.84 (d, J=6.8 Hz, 6H). Mass (m/z): 318.3 [M+H]⁺.

### Compound 401

### N-((3aR, 6aS)-5-(hydroxyamino) octahydropentalen-2-yl)-2-(4-isopropylpiperidin-1-yl) pyrimidin-5-amine

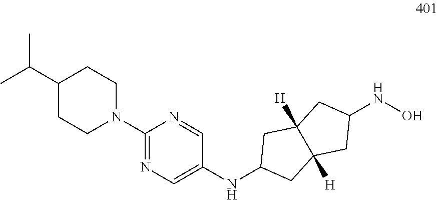 [SMILES: CC(C)C1CCN(c2ncc(NC3C[C@H]4CC(NO)C[C@H]4C3)cn2)CC1]

The title compound 401 (6.9 mg) was prepared in a total yield of 38.3% as a yellow solid according to the procedure for compound 1. ¹H NMR (400 MHZ, Methanol-d₄) δ 7.93 (s, 2H), 4.55-4.46 (m, 2H), 2.78-2.67 (m, 2H), 2.62 (m, 1H), 2.52 (m, 1H), 2.36-2.13 (m, 3H), 1.85 (m, 1H), 1.77-1.68 (m, 3H), 1.61 (m, 1H), 1.48-1.31 (m, 4H), 1.27-1.13 (m, 4H), 0.91 (d, J=6.4 Hz, 6H). Mass (m/z): 360.3 [M+H]⁺.

### Compound 402

### N1-(4-(4-(trifluoromethyl)piperidin-1-yl)phenyl)cyclohexane-1,2-diamine

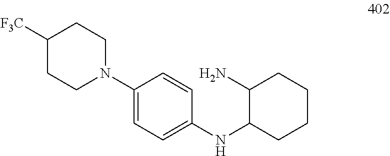 [SMILES: NC1CCCCC1Nc1ccc(N2CCC(C(F)(F)F)CC2)cc1]

The title compound 402 (24.2 mg) was prepared in a total yield of 70.7% as a rosy brown solid according to the procedure for compound 24. ¹H NMR (400 MHZ, Methanol-d₄) δ 6.91 (d, J=8.8 Hz, 2H), 6.74 (d, J=8.4 Hz, 2H), 3.57-3.46 (m, 2H), 3.21 (m, 1H), 2.95 (m, 1H), 2.68-2.57 (m, 2H), 2.25 (m, 1H), 2.17-2.06 (m, 2H), 1.98-1.91 (m, 2H), 1.87-1.63 (m, 4H), 1.56-1.24 (m, 4H). Mass (m/z): 342.2 [M+H]⁺.

### Compound 403

### N1-(6-((2-(4-isopropylpiperidin-1-yl)pyrimidin-5-yl)amino) spiro[3.3]heptan-2-yl)oxalamide

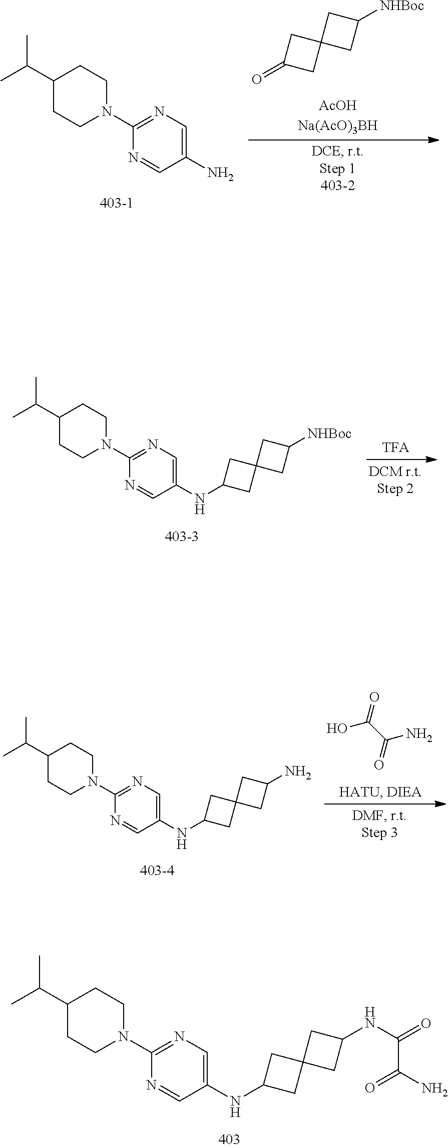 [SMILES: CC(C)C1CCN(c2ncc(N)cn2)CC1 ; CC(C)(C)OC(=O)NC1CC2(CC(=O)C2)C1 ; CC(C)C1CCN(c2ncc(NC3CC4(CC(NC(=O)OC(C)(C)C)C4)C3)cn2)CC1 ; CC(C)C1CCN(c2ncc(NC3CC4(CC(N)C4)C3)cn2)CC1 ; NC(=O)C(=O)O ; CC(C)C1CCN(c2ncc(NC3CC4(CC(NC(=O)C(N)=O)C4)C3)cn2)CC1]

Step 1. Preparation of tert-butyl (6-((2-(4-isopropylpiperidin-1-yl)pyrimidin-5-yl)amino)spiro[3.3]heptan-2-yl)carbamate (403-3)

To a solution of 2-(4-isopropylpiperidin-1-yl)pyrimidin-5-amine (220 mg, 1.0 mmol), tert-butyl (6-oxospiro[3.3]heptan-2-yl)carbamate (225 mg, 1.0 mmol) in DCE (15 mL) was added a drop of AcOH. Then the mixture stirred for 30 mins at r.t. Na(AcO)₃BH (424 mg, 2.0 mmol) was added. The reaction was stirred overnight at rt. The reaction mixture was washed with water (20 mL×3), dried over Na₂SO₄ and concentrated. The residue was purified by prep-TLC (EA/PE=1/2) to afford the desired product (235 mg, 54.8%) as a yellow solid. Mass (m/z): 430.3 [M+H]⁺.

Step 2. Preparation of N²-(2-(4-isopropylpiperidin-1-yl)pyrimidin-5-yl)spiro[3.3]heptane-2,6-diamine (403-4)

To a solution of tert-butyl (6-((2-(4-isopropylpiperidin-1-yl)pyrimidin-5-yl)amino)spiro[3.3]heptan-2-yl)carbamate (235 mg, 0.58 mmol) in DCM (3.0 mL) was added TFA (3.0 mL). Then the solution was stirred for 30 mins at r.t. and concentrated. 5 mL of water was added. The pH of the filtration was adjusted to 8-9 with sodium carbonate solution. Then the mixture was extracted by DCM (5 mL×3). The combined organic layers were washed with water (10 mL), dried over Na₂SO₄ and concentrated. The residue was purified by prep-TLC (MeOH/DCM=1/5) to afford the desired product (176 mg, 92.2%) as a yellow solid. Mass (m/z): 330.2 [M+H]⁺.

Step 3. Preparation of N1-(6-((2-(4-isopropylpiperidin-1-yl)pyrimidin-5-yl)amino)spiro[3.3]heptan-2-yl)oxalamide (403)

To a solution of 2-amino-2-oxoacetic acid (8.9 mg, 0.1 mmol) and HATU (38.0 mg, 0.1 mmol) in DMF (1.0 ml) was added DIEA (38.7 mg, 0.3 mmol). Followed by the addition of N2-(2-(4-isopropylpiperidin-1-yl)pyrimidin-5-yl)spiro[3.3]heptane-2,6-diamine (33.0 mg, 0.1 mmol) then the reaction mixture was stirred for 10 hours at r.t. 5.0 ml of water was added. Then the mixture was extracted by DCM (5.0 mL×3). The combined organic layers were washed with water (5.0 mL×3), dried over Na₂SO₄ and concentrated under vacuum. The residue was purified by prep-TLC (MeOH/DCM=1/10) to give the desired products 403 (10.9 mg, 18.6%) as a yellow solid. ¹H NMR (400 MHZ, DMSO-d₆) δ 8.86 (d, J=8.3 Hz, 1H), 8.01 (s, 1H), 7.79 (s, 2H), 7.74 (s, 1H), 5.24 (d, J=7.4 Hz, 1H), 4.51-4.45 (m, 2H), 4.16-4.05 (m, 1H), 3.66-3.59 (m, 1H), 2.66-2.59 (m, 2H), 2.35-2.10 (m, 6H), 1.84-1.72 (m, 2H), 1.67-1.60 (m, 2H), 1.46-1.36 (m, 1H), 1.22-1.17 (m, 1H), 1.12-1.01 (m, 2H), 0.85 (d, J-6.8 Hz, 6H). Mass (m/z): 401.2 [M+H]⁺.

### Compound 404

### 4-((2-(4-isopropylpiperidin-1-yl)pyrimidin-5-yl)amino)cyclohexane-1-carboxamide

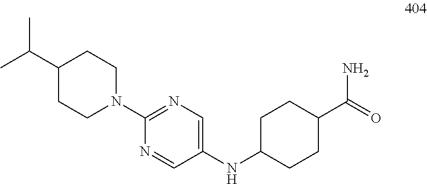 [SMILES: CC(C)C1CCN(c2ncc(NC3CCC(C(N)=O)CC3)cn2)CC1]

The title compound 404 (32.1 mg) was prepared in a yield of 34.7% as a white powder from 2-(4-isopropylpiperidin-1-yl)pyrimidin-5-amine (70 mg, 0.23 mmol) and 4-oxocyclohexane-1-carboxamide (73 mg, 0.35 mmol) according to the procedure for 20. ¹H NMR (400 MHZ, DMSO-d₆) δ 7.92 (s, 1H), 7.89 (s, 1H), 7.17 (d, J=13.3 Hz, 1H), 6.68 (d, J=11.6 Hz, 1H), 4.93 (d, J=8.0 Hz, 1H), 4.53-4.42 (m, 2H), 2.62 (td, J=12.5, 2.4 Hz, 2H), 2.17 (dq, J=9.3, 5.2, 4.5 Hz, 1H), 2.00-1.94 (m, 1H), 1.77 (dd, J=12.5, 9.3 Hz, 2H), 1.64 (d, J=12.6 Hz, 3H), 1.59-1.44 (m, 3H), 1.40 (dt, J=13.3, 6.6 Hz, 1H), 1.25-1.17 (m, 1H), 1.15-1.02 (m, 3H), 0.86 (d, J=6.8 Hz, 6H). Mass (m/z): 346.5 [M+H]⁺.

### Compound 405

### 4-((2-(4-methylpiperidin-1-yl)pyrimidin-5-yl)amino)adamantane-1-carboxamide

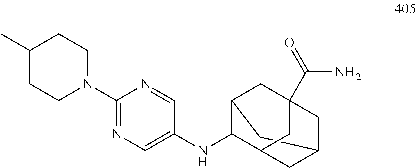 [SMILES: CC1CCN(c2ncc(NC3C4CC5CC3CC(C(N)=O)(C5)C4)cn2)CC1]

The title compound 405 (6.5 mg) was prepared in a yield of 13.5% as a white powder from 2-(4-methylpiperidin-1-yl)pyrimidin-5-amine (40 mg, 0.17 mmol) and 4-oxoadamantane-1-carboxamide (46 mg, 0.24 mmol) according to the procedure for 20. ¹H NMR (400 MHZ, DMSO-d₆) δ 7.91 (d, J=4.0 Hz, 2H), 6.93 (s, 1H), 6.68 (s, 1H), 4.97 (d, J=8.0 Hz, 1H), 4.36 (d, J=12.9 Hz, 2H), 2.73-2.61 (m, 3H), 1.94 (dd, J=26.9, 14.2 Hz, 5H), 1.82 (d, J=13.7 Hz, 4H), 1.71 (s, 2H), 1.57 (d, J=13.2 Hz, 2H), 1.34 (d, J=12.3 Hz, 2H), 1.04-0.93 (m, 2H), 0.87 (d, J=6.4 Hz, 3H). Mass (m/z): 370.3 [M+H]⁺.

### Compound 406

### N1-(5-(4-isopropylpiperidin-1-yl)pyridin-2-yl)cyclohexane-1,4-diamine

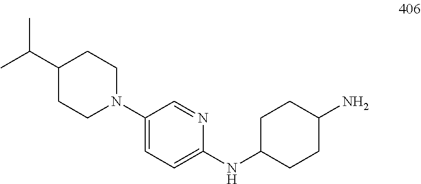 [SMILES: CC(C)C1CCN(c2ccc(NC3CCC(N)CC3)nc2)CC1]

The title compound 406 (21.1 mg, 18.5%) as a light brown solid was prepared from tert-butyl (4-((5-(4-isopropylpiperidin-1-yl)pyridin-2-yl)amino)cyclohexyl)carbamate (150 mg, 0.36 mmol), DCM (2 mL) and TFA (1 mL) according to the procedure for 24. ¹H NMR (400 MHZ, DMSO-d₆) δ 7.59 (t, J=2.8 Hz, 1H), 7.10 (dt, J=9.0, 3.0 Hz, 1H), 6.44-6.29 (m, 1H), 5.73 (dd, J=17.8, 7.6 Hz, 1H), 3.74-3.30 (m, 1H), 2.43-2.35 (m, 2H), 1.91-1.56 (m, 7H), 1.54-1.34 (m, 3H), 1.32-1.17 (m, 3H), 1.17-0.97 (m, 4H), 0.84 (d, J=6.8 Hz, 6H). MS (m/z): 317.3 [M+H]⁺.

### Compound 407

### N-(((1r,4r)-4-aminocyclohexyl)methyl)-4-(2,6-dimethylmorpholino)-2-methylaniline

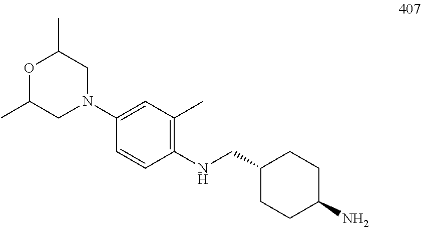 [SMILES: Cc1cc(N2CC(C)OC(C)C2)ccc1NC[C@H]1CC[C@H](N)CC1]

The title compound 407 (61.2 mg) was prepared in a total yield of 65.3% as a white solid from tert-butyl ((1r,4r)-4-(((4-(2,6-dimethylmorpholino)-2-methylphenyl)amino)methyl)cyclohexyl)carbamate (122 mg, 0.283 mmol), TFA (1 mL), and DCM (10 mL) according to the procedure for 24. ¹H NMR (400 MHZ, DMSO-d₆) δ 8.30-8.15 (m, 3H), 6.74 (d, J=8.4 Hz, 1H), 6.36 (s, 2H), 3.70-3.59 (m, 2H), 2.85 (s, 1H), 2.72 (dd, J=22.0, 8.8 Hz, 4H), 2.19 (t, J=10.8 Hz, 2H), 2.11 (s, 3H), 1.98-1.90 (m, 2H), 1.81 (d, J=12.8 Hz, 2H), 1.41 (s, 1H), 1.35-1.23 (m, 2H), 1.04 (d, J=6.4 Hz, 6H), 1.00-0.88 (m, 2H). Mass (m/z): 332.3 [M+H]⁺.

### Compound 408

### 1-(4-((2-(4-isopropylpiperidin-1-yl)pyrimidin-5-yl)amino) adamantan-1-yl)urea

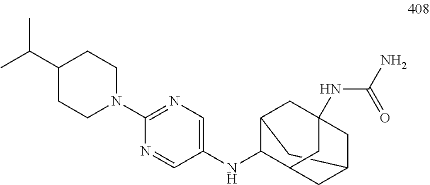 [SMILES: CC(C)C1CCN(c2ncc(NC3C4CC5CC3CC(NC(N)=O)(C5)C4)cn2)CC1]

The title compound 408 (6.5 mg) was prepared in a yield of 13.5% as a white powder from N⁴-(2-(4-isopropylpiperidin-1-yl)pyrimidin-5-yl)adamantane-1,4-diamine (46 mg, 0.18 mmol) and phenyl carbamate (24 mg, 0.17 mmol) according to the procedure for 243. ¹H NMR (400 MHz, DMSO-d₆) δ 7.94 (d, J=4.3 Hz, 2H), 5.89 (s, 1H), 5.72 (s, 1H), 4.48 (d, J=12.9 Hz, 2H), 2.62 (td, J=12.8, 2.2 Hz, 2H), 2.40 (d, J=3.8 Hz, 3H), 2.20-2.09 (m, 6H), 2.11-2.04 (m, 4H), 1.68-1.59 (m, 3H), 1.47-1.31 (m, 2H), 1.08 (qd, J=12.2, 4.1 Hz, 2H), 0.86 (d, J=6.7 Hz, 6H). Mass (m/z): 413.5 [M+H]⁺.

Compound 409

### 1-((1r,4r)-4-(((2-(4-isopropylpiperidin-1-yl)pyrimidin-5-yl)amino)methyl)cyclohexyl)urea

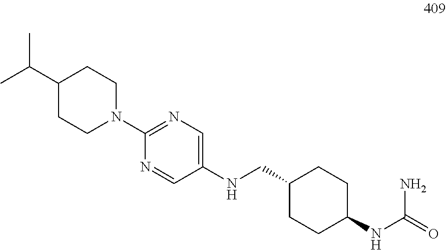 [SMILES: CC(C)C1CCN(c2ncc(NC[C@H]3CC[C@H](NC(N)=O)CC3)cn2)CC1]

The title compound 409 (6.9 mg) was prepared in a yield of 14.2% as a white powder from N-(((1r,4r)-4-aminocyclohexyl)methyl)-2-(4-isopropylpiperidin-1-yl)pyrimidin-5-amine (51 mg, 0.19 mmol) and phenyl carbamate (25 mg, 0.17 mmol) according to the procedure for 243. 1¹H NMR (400 MHZ, DMSO-d₆) δ 7.86 (s, 2H), 5.76 (d, J=8.0 Hz, 1H), 5.06 (t, J=6.1 Hz, 1H), 4.46 (d, J=13.2 Hz, 2H), 3.23 (s, 1H), 2.78 (t, J=6.3 Hz, 2H), 2.61 (t, J=12.1 Hz, 2H), 1.78 (d, J=11.6 Hz, 4H), 1.63 (d, J=11.9 Hz, 2H), 1.39 (dd, J=13.0, 6.7 Hz, 2H), 1.16 (s, 1H), 1.08 (td, J=12.3, 4.0 Hz, 2H), 0.99 (q, J=11.8, 11.0 Hz, 4H), 0.84 (d, J=6.8 Hz, 6H). Mass (m/z): 375.4 [M+H]⁺

### Compound 410

### 4-((2-(4,4-dimethylpiperidin-1-yl)pyrimidin-5-yl)amino)adamantane-1-carboxamide

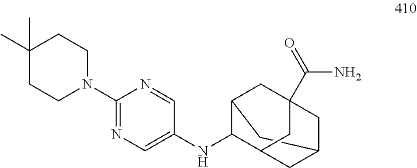 [SMILES: CC1(C)CCN(c2ncc(NC3C4CC5CC3CC(C(N)=O)(C5)C4)cn2)CC1]

The title compound 410 (8.2 mg) was prepared in a yield of 12.09% as a white powder from 2-(4,4-dimethylpiperidin-1-yl)pyrimidin-5-amine (68 mg, 0.18 and mmol) 4-oxoadamantane-1-carboxamide (41 mg, 0.25 mmol) according to the procedure for 20. ¹H NMR (400 MHZ, DMSO-d₆) δ 7.95 (d, J=4.1 Hz, 2H), 6.98 (s, 1H), 6.73 (s, 1H), 5.01 (d, J=7.8 Hz, 1H), 3.60-3.50 (m, 4H), 2.89 (s, 2H), 2.73 (s, 1H), 2.03-1.91 (m, 2H), 1.89-1.79 (m, 3H), 1.75 (d, J=2.9 Hz, 3H), 1.38 (d, J=12.4 Hz, 2H), 1.33-1.25 (m, 4H), 0.94 (s, 6H). Mass (m/z): 384.2 [M+H]⁺.

### Compound 411

### N1-(6-(4-isopropylpiperidin-1-yl)pyridazin-3-yl)cyclohexane-1,4-diamine

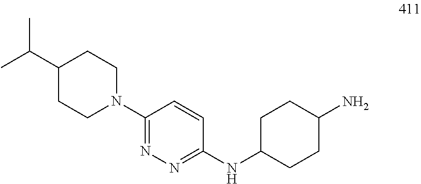 [SMILES: CC(C)C1CCN(c2ccc(NC3CCC(N)CC3)nn2)CC1]

The title compound 411 (8.4 mg, 17.5%) as an off-white solid was prepared from tert-butyl (4-((6-(4-isopropylpiperidin-1-yl)pyridazin-3-yl)amino)cyclohexyl)carbamate (60 mg, 0.14 mmol), DCM (2 mL) and TFA (1 mL) according to the procedure for 24. ¹H NMR (400 MHZ, DMSO-d₆) δ 7.08 (dd, J=9.7, 2.3 Hz, 1H), 6.72 (dd, J=39.5, 9.6 Hz, 1H), 5.93 (dd, J=12.3, 7.2 Hz, 1H), 4.00 (d, J=12.3 Hz, 2H), 3.90-3.51 (m, 1H), 3.04-2.72 (m, 1H), 2.64-2.57 (m, 2H), 2.00-1.96 (m, 1H), 1.80-1.76 (m, 1H), 1.75-1.61 (m, 3H), 1.61-1.48 (m, 1H), 1.46-1.37 (m, 2H), 1.28-1.05 (m, 6H), 0.87 (d, J=6.8 Hz, 6H). MS (m/z): 318.3 [M+H]⁺.

### Compound 412

### N2-(2-fluoroethyl)-N6-(2-(4-isopropylpiperidin-1-yl)pyrimidin-5-yl)spiro[3.3]heptane-2,6-diamine

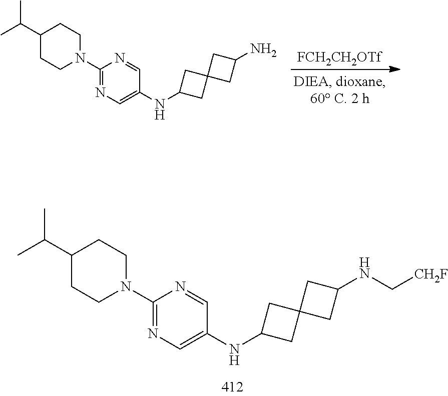

TEA (92 mg, 0.91 mmol), 2-fluoroethyl trifluoromethanesulfonate (119 mg, 0.61 mmol) was added a to solution of 2-N-[2-(4-isopropylpiperidin-1-yl)pyrimidin-5-yl]spiro[3.3]heptane-2,6-diamine hydrochloride (100 mg, 0.31 mmol) in dioxane (20 mL), the mixture was heated to 60° C. for 2 hrs, then the mixture was removed in vacuo. The residue was purified by prep-HPLC (column-Gemini-C18 150×21.2 mm, 5 μm; Mobile phase: ACN-H₂O (0.1% FA), 5%-20%) to afford the desired product 412 (6.7 mg, 5.8%) as yellow solid. ¹H NMR (400 MHZ, CD₃OD) δ 7.81 (s, 2H), 4.73-4.70 (m, 1H), 4.62-4.57 (m, 1H), 4.47 (d, J=13.2 Hz, 2H), 3.69 (dt, J=11.8, 7.8 Hz, 2H), 3.22 (s, 1H), 3.18-3.14 (m, 1H), 2.71 (dd, J=18.0, 7.2 Hz, 2H), 2.60-2.51 (m, 2H), 2.45-2.33 (m, 2H), 2.23-2.14 (m, 2H), 1.97-1.89 (m, 2H), 1.70 (d, J=11.9 Hz, 2H), 1.45-1.37 (m, 1H), 1.26-1.11 (m, 3H), 0.88 (d, J=6.8 Hz, 6H). MS (m/z): 376.3 [M+H]⁺

### Compound 413

### N2-(2,2-difluoroethyl)-N6-(2-(4-isopropylpiperidin-1-yl)pyrimidin-5-yl)spiro[3.3]heptane-2,6-diamine

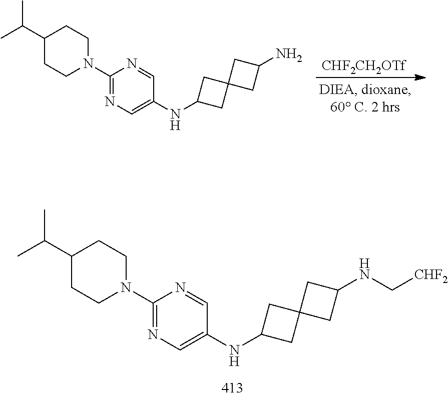 [SMILES: CC(C)C1CCN(c2ncc(NC3CC4(CC(N)C4)C3)cn2)CC1 ; CC(C)C1CCN(c2ncc(NC3CC4(CC(NCC(F)F)C4)C3)cn2)CC1 ; "CHF2CH2OTf"]

DIEA (118 mg, 0.91 mmol), 2,2-Difluoroethyl trifluoromethanesulfonate (130 mg, 0.61 mmol), was added to a solution of 2-N-[2-(4-isopropylpiperidin-1-yl)pyrimidin-5-yl]spiro[3.3]heptane-2,6-diamine hydrochloride (100 mg, 0.31 mmol) in MeOH (20 mL), the mixture was stirred at rt for 2 hrs, then the mixture was removed in vacuo. The residue was purified by prep-HPLC (column-Gemini-C18 150×21.2 mm, 5 μm; Mobile phase: ACN-H₂O (0.1% FA), 5%-20%) to afford the desired product as yellow solid (8.7 mg, 7.2%). ¹H NMR (400 MHZ, CD₃OD) δ 7.81 (s, 2H), 6.13 (dd, J=55.6, 52.9 Hz, 1H), 4.47 (d, J=13.2 Hz, 2H), 3.73-3.55 (m, 2H), 3.28-3.19 (m, 2H), 2.71 (dd, J=18.0, 7.2 Hz, 2H), 2.57-2.47 (m, 2H), 2.43-2.30 (m, 2H), 2.17-2.08 (m, 2H), 1.91 (dt, J=11.2, 8.3 Hz, 2H), 1.70 (d, J=11.8 Hz, 2H), 1.44-1.37 (m, 1H), 1.18 (m, 3H), 0.88 (d, J=6.8 Hz, 6H). MS (m/z): 394.2 [M+H]⁺

### Compound 414

### N2-(2-(4-isopropylpiperidin-1-yl)pyrimidin-5-yl)-N6-(2,2, 2-trifluoroethyl)spiro[3.3]heptane-2, 6-diamine

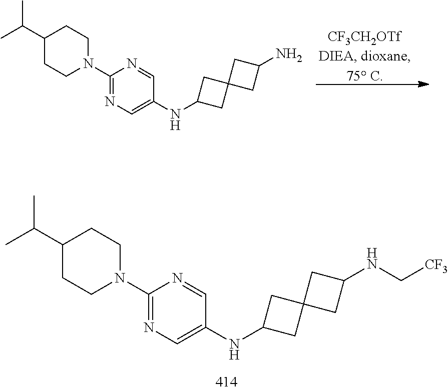 [SMILES: CC(C)C1CCN(c2ncc(NC3CC4(CC(N)C4)C3)cn2)CC1 ; "CF3CH2OTfDIEA, dioxane, 75° C." ; CC(C)C1CCN(c2ncc(NC3CC4(CC(NCC(F)(F)F)C4)C3)cn2)CC1]

DIEA (106 mg, 0.82 mmol), 2,2,2-trifluoroethyl triflate (127 mg, 0.55 mmol), was added to a solution of 2-N-[2-(4-isopropylpiperidin-1-yl)pyrimidin-5-yl]spiro[3.3]heptane-2,6-diamine hydrochloride (100 mg, 0.27 mmol), in dioxane (20 mL), and the mixture was stirred at rt for 2 hrs, and then the mixture was removed in vacuo. The residue was purified by prep-HPLC (column-Gemini-C18 150×21.2 mm, 5 μm; Mobile phase: ACN-H₂O (0.1% FA), 5%-20%) to afford the desired product (3.3 mg, 2.9%) as yellow solid. ¹H NMR (400 MHZ, CD₃OD) δ 7.81 (d, J=0.7 Hz, 2H), 4.49-4.44 (m, 2H), 3.66 (t, J=7.5 Hz, 1H), 3.21 (d, J=7.5 Hz, 1H), 3.09 (d, J=9.9 Hz, 2H), 2.74-2.67 (m, 2H), 2.48 (ddd, J=11.3, 6.3, 2.8 Hz, 1H), 2.41-2.29 (m, 2H), 2.22-2.16 (m, 1H), 1.87-1.78 (m, 4H), 1.73-1.68 (m, 2H), 1.45-1.38 (m, 1H), 1.19 (m, 3H), 0.88 (d, J=6.8 Hz, 6H). MS (m/z): 412.3 [M+H]⁺.

### Compound 415

### 2-((4-(4-(trifluoromethyl)piperidin-1-yl)phenyl)amino)cyclohexan-1-ol

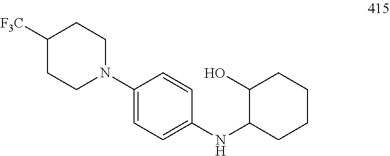 [SMILES: OC1CCCCC1Nc1ccc(N2CCC(C(F)(F)F)CC2)cc1]

The title compound 415 (20.6 mg) was prepared in a total yield of 60.1% as a white solid according to the procedure for compound 4. ¹H NMR (400 MHZ, DMSO-d₆) δ 6.74 (d, J=8.8 Hz, 2H), 6.52 (d, J=8.0 Hz, 2H), 4.48 (m, 1H), 3.47-3.36 (m, 2H), 3.23 (m, 1H), 2.42-2.30 (m, 2H), 1.96 (m, 1H), 1.90-1.81 (m, 2H), 1.75-1.43 (m, 6H), 1.35-1.23 (m, 4H). Mass (m/z): 343.2 [M+H]⁺.

### Compound 416

### (5-((4-aminocyclohexyl)amino)-2-(4-(trifluoromethyl)piperidin-1-yl) phenoxy) ethan-1-ol

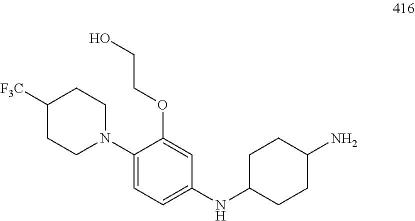 [SMILES: NC1CCC(Nc2ccc(N3CCC(C(F)(F)F)CC3)c(OCCO)c2)CC1]

The title compound 416 (43.7 mg) was prepared in a total yield of 72.5% as a white solid according to the procedure for compound 24. ¹H NMR (400 MHZ, DMSO-d₆) δ 6.66 (s, 1H), 6.23 (d, J=8.8 Hz, 1H), 6.09 (d, J=8.4 Hz, 1H), 3.90 (m, 2H), 3.67 (m, 2H), 3.30-3.21 (m, 2H), 3.06 (m, 1H), 2.96 (m, 1H), 2.42-2.31 (m, 2H), 2.05-1.90 (m, 2H), 1.87-1.34 (m, 10H), 1.26-1.09 (m, 1H). Mass (m/z): 402.2 [M+H]⁺.

### Compound 417

### (1-(4-((4-aminocyclohexyl)amino)phenyl)piperidin-2-yl)methanol

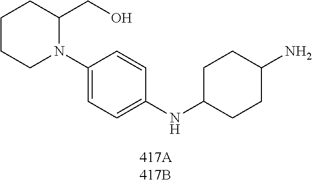 [SMILES: NC1CCC(Nc2ccc(N3CCCCC3CO)cc2)CC1]

The title compound 417 was prepared according to the procedure for compound 24. The mixture was purified by prep HPLC (XSelect-CSH-Prep 5 μm OBD, 19*150 mm column; ACN/water (0.5% TFA)=10%-35%-95%-95%-0%, 0 min-10 min-10.5 min-11.5 min-13 min) to afford compound 417A (Rt=8.52 min) in 37.8% yield as a gray solid and 417B (Rt=9.64 min) in 12.2% yield as a gray solid. 417A: ¹H NMR (400 MHZ, Methanol-d₄) δ 7.01 (d, J=8.8 Hz, 2H), 6.74 (d, J=8.8 Hz, 2H), 3.61-3.40 (m, 2H), 3.22-2.93 (m, 3H), 2.85 (m, 1H), 2.76 (m, 1H), 2.05-1.80 (m, 4H), 1.63-1.54 (m, 2H), 1.50-1.20 (m, 6H), 1.18-1.03 (m, 2H). Mass (m/z): 304.2 [M+H]⁺. 417B: ¹H NMR (400 MHZ, Methanol-d₄) δ 7.02 (d, J=8.8 Hz, 2H), 6.74 (d, J=8.8 Hz, 2H), 3.50-3.35 (m, 2H), 3.21-2.95 (m, 3H), 2.84 (m, 1H), 2.76 (m, 1H), 2.05-1.91 (m, 3H), 1.85-1.63 (m, 4H), 1.60-1.53 (m, 3H), 1.23-1.05 (m, 4H). Mass (m/z): 304.2 [M+H]⁺.

### Compound 418

### N-(((1r,4r)-4-aminocyclohexyl)methyl)-2-fluoro-6-(4-(trifluoromethyl)piperidin-1-yl)pyridin-3-amine

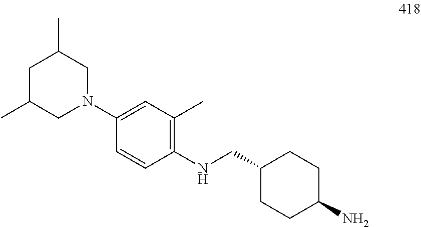 [SMILES: Cc1cc(N2CC(C)CC(C)C2)ccc1NC[C@H]1CC[C@H](N)CC1]

The title compound 418 (14.1 mg, 22%) as a yellow solid was prepared from ((1r,4r)-4-(((4-(3,5-dimethylpiperidin-1-yl)-2-methylphenyl)amino)methyl)cyclohexyl)carbamate (100 mg, 0.23 mmol), DCM (5 mL) and TFA (1 mL) according to the procedure for 24. ¹H NMR (400 MHZ, DMSO-d₆) δ=6.65 (s, 2H), 6.38 (s, 1H), 4.25 (s, 1H), 3.30 (s, 9H), 2.83 (s, 2H), 2.04-1.97 (m, 3H), 1.80-1.71 (m, 4H), 1.48 (s, 1H), 1.24 (s, 2H), 1.08 (s, 1H), 0.87 (s, 6H). Mass (m/z): 329.9 [M+H]⁺.

### Compound 419

### N-(((1r,4r)-4-aminocyclohexyl)methyl)-2-methyl-4-(3-(trifluoromethyl) pyrrolidin-1-yl)aniline

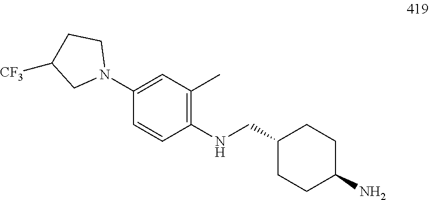 [SMILES: Cc1cc(N2CCC(C(F)(F)F)C2)ccc1NC[C@H]1CC[C@H](N)CC1]

The title compound 419 (45.1 mg, 35.8%) as a purple solid was prepared from tert-butyl ((11,4r)-4-(((2-methyl-4-(3-(trifluoromethyl) pyrrolidin-1-yl)phenyl)amino)methyl)cyclohexyl)carbamate (160 mg, 0.35 mmol), DCM (10 mL) and TFA (1 mL) according to the procedure for 24. ¹H NMR (400 MHZ, DMSO-d₆) δ 6.40 (m, 3H), 3.43-3.28 (m, 2H), 3.23-3.11 (m, 3H), 2.91 (m, 1H), 2.83 (d, J=6.6 Hz, 2H), 2.25-2.18 (m, 1H), 2.06 (s, 3H), 2.02-1.84 (m, 5H), 1.52 (s, 1H), 1.30-1.20 (m, 2H), 1.00 (m, 2H). Mass (m/z): 356.22 [M+H]⁺.

### Compound 420

### 4-(2,6-dimethylmorpholino)-N-(((1r,4r)-4-(methylamino)cyclohexyl)methyl)aniline

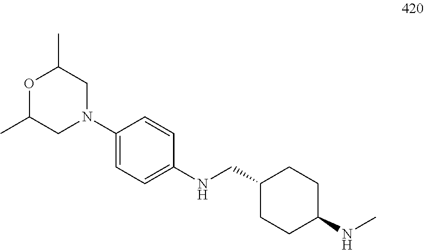

The title compound 420 (4.2 mg, 5%) as a white solid was prepared from tert-butyl ((1r,4r)-4-(((4-(2,6-dimethylmorpholino)phenyl)amino)methyl)cyclohexyl)carbamate (100 mg, 0.24 mmol), THF (3 mL) and LAH (0.3 mL, 0.71 mmol) according to the procedure for 23. H NMR (400 MHZ, DMSO-d₆) δ 6.73-6.64 (m, 2H), 6.48-6.39 (m, 2H), 5.06 (s, 1H), 3.69-3.57 (m, 2H), 3.25-3.17 (m, 2H), 2.74 (t, J=5.4 Hz, 2H), 2.27 (s, 3H), 2.12-2.02 (m, 2H), 1.91-1.83 (m, 2H), 1.82-1.75 (m, 2H), 1.43-1.39 (m, 1H), 1.22-1.18 (m, 1H), 1.08 (d, J=6.3 Hz, 6H), 1.01-0.78 (m, 4H). MS (m/z): 332.3 [M+H]⁺.

### Compound 421

### N⁴-(4-(2,6-dimethylmorpholino)phenyl)adamantane-1,4-diamine

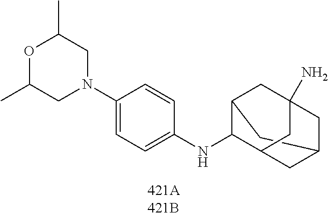 [SMILES: CC1CN(c2ccc(NC3C4CC5CC3CC(N)(C5)C4)cc2)CC(C)O1]

The title compound 421A (Rt=7.5 min) (17 mg, 21.7%) as a white solid and compound 421B (Rt=8.76 min) (11.4 mg, 14.6%) as a white solid were prepared from tert-butyl (4-((4-(2,6-dimethylmorpholino)phenyl)amino) adamantan-1-yl)carbamate (100 mg, 0.22 mmol), DCM (3 mL), and TFA (1 mL) according to the procedure for 24, which were purified by Prep-HPLC (XSelect-CSH-Prep 5 μm OBD, 19*150 mm column; ACN/water (0.5% TFA)=0%-30%-95%-95%-0%, 0 min-10 min-10.5 min-11.5 min-13 min). 421A: ¹H NMR (400 MHz, DMSO-d₆) δ 6.72-6.64 (m, 2H), 6.55-6.47 (m, 2H), 4.94 (d, J=7.5 Hz, 1H), 3.69-3.57 (m, 2H), 3.27-3.19 (m, 3H), 2.12-2.02 (m, 2H), 1.93-1.85 (m, 5H), 1.74-1.45 (m, 7H), 1.27-1.18 (m, 3H), 1.08 (d, J=6.2 Hz, 6H). MS (m/z): 356.3 [M+H]⁺. 421B: ¹H NMR (400 MHZ, DMSO-d₆) δ 6.72-6.64 (m, 2H), 6.56-6.47 (m, 2H), 4.88 (d, J=7.3 Hz, 1H), 3.69-3.57 (m, 2H), 3.25-3.15 (m, 3H), 2.12-2.02 (m, 2H), 2.00-1.89 (m, 3H), 1.77 (d, J=12.2 Hz, 2H), 1.68-1.51 (m, 5H), 1.46-1.40 (m, 2H), 1.23-1.16 (m, 3H), 1.08 (d, J=6.3 Hz, 6H). MS (m/z): 356.3 [M+H]⁺.

### Compound 422

### 6-((2-(4-isopropylpiperidin-1-yl)pyrimidin-5-yl)amino)spiro[3.3]heptan-2-ol

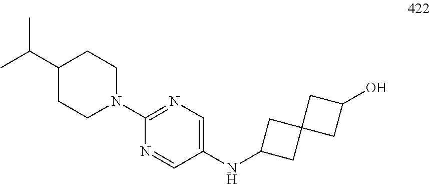 [SMILES: CC(C)C1CCN(c2ncc(NC3CC4(CC(O)C4)C3)cn2)CC1]

The title compound 422 (32.3 mg) was prepared in a yield of 43.07% as a white powder from 2-(4-isopropylpiperidin-1-yl)pyrimidin-5-amine (50 mg, 0.23 and mmol) 6-hydroxyspiro [3.3]heptan-2-one (34 mg, 0.27 mmol) according to the procedure for 20. ¹H NMR (400 MHZ, DMSO-d₆) δ 7.77 (s, 2H), 5.22 (d, J=7.5 Hz, 1H), 4.88 (d, J=6.4 Hz, 1H), 4.47 (dt, J=12.4, 3.0 Hz, 2H), 3.99-3.90 (m, 1H), 3.61 (q, J=7.5 Hz, 1H), 2.61 (td, J=12.6, 2.5 Hz, 2H), 2.53 (dd, J=7.7, 1.9 Hz, 1H), 2.40-2.31 (m, 2H), 2.27 (ddd, J=11.5, 7.1, 4.7 Hz, 1H), 2.15 (dt, J=11.7, 6.2 Hz, 1H), 1.85-1.78 (m, 2H), 1.74 (ddd, J=11.2, 7.9, 3.3 Hz, 2H), 1.67-1.60 (m, 2H), 1.44-1.33 (m, 1H), 1.20 (ddt, J=14.7, 6.1, 3.0 Hz, 1H), 1.06 (qd, J=12.3, 4.2 Hz, 2H), 0.85 (d, J=6.8 Hz, 6H). Mass (m/z): 331.4 [M+H]⁺.

### Compound 423

### (1r,4r)-4-(((2-(4-isopropylpiperidin-1-yl)pyrimidin-5-yl)amino)methyl)cyclohexane-1-carboxamide

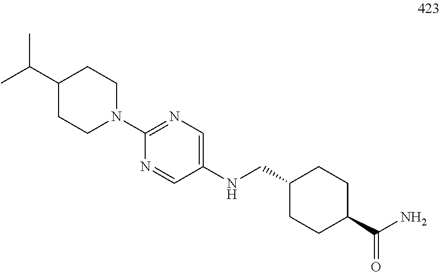 [SMILES: CC(C)C1CCN(c2ncc(NC[C@H]3CC[C@H](C(N)=O)CC3)cn2)CC1]

The title compound 423 (8.5 mg) was prepared in a yield of 17.0% as a white powder from (1r,4r)-4-(((2-(4-isopropylpiperidin-1-yl)pyrimidin-5-yl)amino)methyl)cyclohexane-1-carboxylic acid (50 mg, 0.14 mmol) and ammonia (0.500 mL) according to the procedure for 10. ¹H NMR (400 MHZ, DMSO-d₆) δ 7.87 (s, 1H), 7.16 (s, 1H), 6.64 (s, 1H), 4.54 (dd, J=53.1, 13.4 Hz, 2H), 2.79 (t, J=6.4 Hz, 1H), 2.66-2.57 (m, 2H), 2.01 (t, J=6.1 Hz, 1H), 1.74 (ddd, J=46.9, 30.3, 12.6 Hz, 8H), 1.41 (dt, J=13.4, 6.6 Hz, 3H), 1.24 (s, 7H), 1.16-1.00 (m, 1H), 0.96-0.81 (m, 6H). Mass (m/z): 360.5 [M+H]⁺.

### Compound 424

### 4-((2-(4-isopropylpiperidin-1-yl)pyrimidin-5-yl)amino) adamantan-1-ol

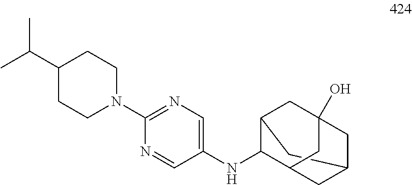 [SMILES: CC(C)C1CCN(c2ncc(NC3C4CC5CC3CC(O)(C5)C4)cn2)CC1]

The title compound 424 (28.6 mg) was prepared in a yield of 34.01% as a white powder from 2-(4-isopropylpiperidin-1-yl)pyrimidin-5-amine (50) mg, 0.23 mmol) and 5-hydroxyadamantan-2-one (45 mg, 0.27 mmol) according to the procedure for 20. ¹H NMR (400 MHZ, DMSO-d₆) δ 7.90 (d, J=1.9 Hz, 2H), 4.92 (dd, J=12.4, 7.7 Hz, 1H), 4.44 (dt, J=12.7, 2.7 Hz, 2H), 4.33 (d, J=18.2 Hz, 1H), 2.58 (td, J=12.6, 2.5 Hz, 2H), 1.98 (d, J=30.4 Hz, 3H), 1.86 (d, J=12.7 Hz, 2H), 1.69 (d, J=11.6 Hz, 1H), 1.65-1.49 (m, 7H), 1.40-1.33 (m, 1H), 1.33-1.13 (m, 2H), 1.04 (qd, J=12.2, 4.1 Hz, 2H), 0.82 (d, J=6.8 Hz, 6H). Mass (m/z): 371.4 [M+H]⁺.

### Compound 425

### N1-(6-((2-(3,5-dimethylpiperidin-1-yl)pyrimidin-5-yl)amino)spiro[3.3]heptan-2-yl)oxalamide

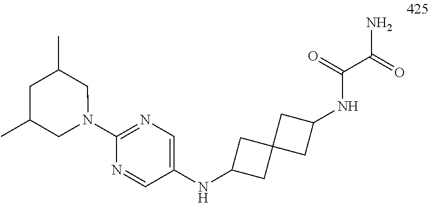 [SMILES: CC1CC(C)CN(c2ncc(NC3CC4(CC(NC(=O)C(N)=O)C4)C3)cn2)C1]

The title compound 425 (4.8 mg) was prepared in a yield of 21.7% as a white powder from N2-(2-(3,5-dimethylpiperidin-1-yl)pyrimidin-5-yl)spiro[3.3]heptane-2,6-diamine (18 mg, 0.057 mmol) and 2-amino-2-oxoacetic acid (6 mg, 0.068 mmol) according to the procedure for 10. ¹H NMR (400 MHZ, DMSO-d₆) δ 8.85 (d, J=8.3 Hz, 1H), 8.01 (s, 1H), 7.78 (s, 1H), 7.73 (s, 1H), 4.42 (d, J=13.8 Hz, 2H), 3.68-3.54 (m, 3H), 3.17-3.08 (m, 3H), 2.25-2.09 (m, 2H), 1.73 (d, J=10.5 Hz, 2H), 1.19 (d, J=6.7 Hz, 6H), 0.85 (dd, J=6.7, 3.5 Hz, 6H). Mass (m/z): 387.4 [M+H]⁺.

### Compound 426

### 5-(aminomethyl)-N-(4-(2,6-dimethylmorpholino)phenyl)adamantan-2-amine

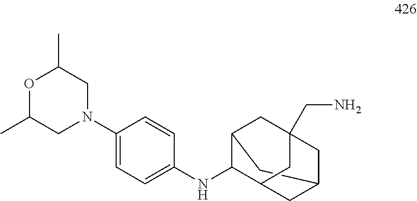 [SMILES: CC1CN(c2ccc(NC3C4CC5CC3CC(CN)(C5)C4)cc2)CC(C)O1]

The title compound 426 (28.2 mg) was prepared in a two-step overall yield of 26.7% as a white powder from 4-(2,6-dimethylmorpholino) aniline (70 mg, 0.34 mmol) and 4-oxoadamantane-1-carboxylic acid (99 mg, 0.51 mmol) according to the procedure for 84. ¹H NMR (400 MHZ, DMSO-d₆) δ 7.91 (s, 2H), 6.75 (s, 2H), 6.58 (s, 2H), 5.08 (s, 1H), 3.68 (s, 2H), 2.12 (s, 2H), 1.98 (s, 4H), 1.91 (d, J=6.6 Hz, 2H), 1.72 (d, J=17.9 Hz, 2H), 1.63 (s, 3H), 1.53 (s, 2H), 1.49 (s, 1H), 1.35 (d, J=12.2 Hz, 1H), 1.25 (d, J=7.8 Hz, 1H), 1.12 (d, J=6.0 Hz, 6H). Mass (m/z): 389.3 [M+H]⁺.

### Compound 427

### 6-((2-(4-(trifluoromethyl)piperidin-1-yl)pyrimidin-5-yl)amino)spiro[3.3]heptane-2-carbohydrazide

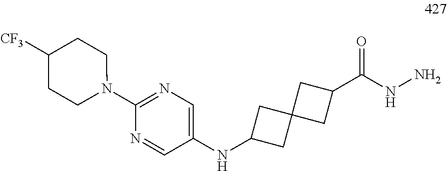 [SMILES: NNC(=O)C1CC2(CC(Nc3cnc(N4CCC(C(F)(F)F)CC4)nc3)C2)C1]

The title compound 427 (5.1 mg) was prepared in a total yield of 16.4% as a yellow solid from 6-((2-(4-(trifluoromethyl)piperidin-1-yl)pyrimidin-5-yl)amino)spiro[3.3]heptane-2-carboxylic acid (30 mg, 0.078 mmol), hydrazinium hydroxide solution (23 mg, 0.468 mmol), HATU (45 mg, 0.117 mmol) and DIEA (30 mg, 0.234 mmol) and DMF (5 mL) according to the procedure for 1. ¹H NMR (400 MHZ, DMSO-d₆) δ 9.01 (s, 1H), 8.92 (s, 1H), 7.78 (s, 2H), 4.54-4.11 (m, 7H), 3.63-3.51 (m, 2H), 2.80 (t, J=8.4 Hz, 1H), 2.72 (td, J=12.8, 2.5 Hz, 2H), 2.25 (td, J=6.8, 3.4 Hz, 1H), 2.18-2.03 (m, 3H), 1.93-1.87 (m, 1H), 1.77 (dd, J=12.4, 3.8 Hz, 3H), 1.71 (s, 3H), 1.67 (dd, J=11.2, 7.8 Hz, 1H), 1.28 (tt, J=12.0, 5.2 Hz, 8H), 0.86-0.78 (m, 1H). Mass (m/z): 399.3 [M+H]⁺.

### Compound 428

### N-hydroxy-6-((2-(4-(trifluoromethyl)piperidin-1-yl)pyrimidin-5-yl)amino)spiro[3.3]heptane-2-carboxamide

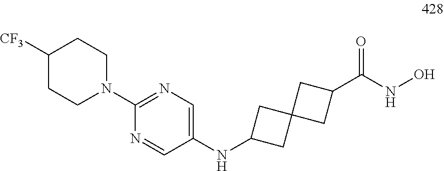 [SMILES: O=C(NO)C1CC2(CC(Nc3cnc(N4CCC(C(F)(F)F)CC4)nc3)C2)C1]

The title compound 428 (7.0 mg) was prepared in a total yield of 22.4% as a yellow solid from 6-((2-(4-(trifluoromethyl)piperidin-1-yl)pyrimidin-5-yl)amino)spiro[3.3]heptane-2-carboxylic acid (30 mg, 0.078 mmol), hydroxylamine hydrochloride (11 mg, 0.156 mmol), HATU (45 mg, 0.117 mmol) and DIEA (30 mg, 0.234 mmol) and DMF (5 mL) according to the procedure for 1. ¹H NMR (400 MHz, DMSO-d₆) δ 10.37 (s, 1H), 8.65 (d, J=1.6 Hz, 1H), 7.78 (s, 2H), 5.36 (d, J=7.2 Hz, 1H), 4.49 (d, J=13.2 Hz, 2H), 3.57 (q, J=7.2 Hz, 1H), 2.78-2.68 (m, 3H), 2.26 (q, J=5.6, 5.2 Hz, 1H), 2.11 (ddd, J=22.0, 11.2, 8.4 Hz, 3H), 1.89 (ddd, J=12.0, 8.0, 4.0 Hz, 1H), 1.77 (dd, J=12.0, 4.0 Hz, 3H), 1.67 (dd, J=11.2, 8.0 Hz, 1H), 1.30 (qd, J=12.4, 4.0 Hz, 4H). Mass (m/z): 400.3 [M+H]⁺.

### Compound 429

### 1-((1r,4r)-4-(2-((2-(4-isopropylpiperidin-1-yl)pyrimidin-5-yl)amino)ethyl)cyclohexyl)urea

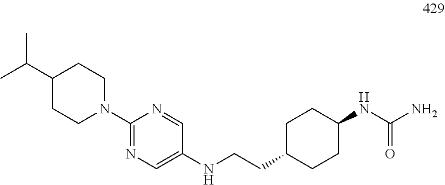 [SMILES: CC(C)C1CCN(c2ncc(NCC[C@H]3CC[C@H](NC(N)=O)CC3)cn2)CC1]

The title compound 429 (4.4 mg, 3.7%) was prepared from of N-(2-((1r,4r)-4-aminocyclohexyl)ethyl)-2-(4-isopropylpiperidin-1-yl)pyrimidin-5-amine (100 mg, 0.2894 mmol), DCM (10 mL), TMSNCO (33.34 mg, 0.2894 mmol), TEA (87.85 mg, 0.8682 mmol) and DMAP (7.07 mg, 0.0579 mmol) according to the procedure for 178. ¹H NMR (400 MHZ, DMSO-d₆) δ 7.86 (s, 2H), 5.78 (d, J=8.0 Hz, 1H), 5.28 (s, 2H), 4.94 (s, 1H), 4.48 (d, J=13.0 Hz, 2H), 3.25-3.19 (m, 1H), 2.94 (t, J=6.9 Hz, 2H), 2.67-2.58 (m, 2H), 1.83-1.61 (m, 6H), 1.46-1.36 (m, 3H), 1.13 (m, 8H), 0.85 (d, J=6.8 Hz, 6H). Mass (m/z): 389.30 [M+H]⁺.

### Compound 430

### N-(((1r,4r)-4-aminocyclohexyl)methyl)-4-(2,6-dimethylmorpholino)-3-fluoroaniline

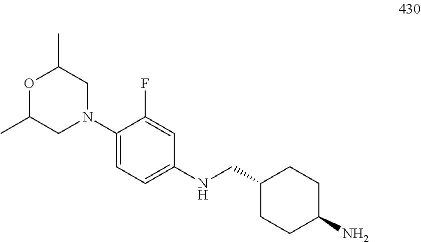 [SMILES: CC1CN(c2ccc(NC[C@H]3CC[C@H](N)CC3)cc2F)CC(C)O1]

The title compound 430 (10 mg, 12.9%) as a white solid was prepared from tert-butyl ((1r,4r)-4-(((4-(2,6-dimethylmorpholino)-3-fluorophenyl)amino)methyl)cyclohexyl)carbamate (100 mg, 0.23 mmol), DCM (3 mL) and TFA (1 mL) according to the procedure for 24. ¹H NMR (400 MHZ, DMSO-d₆) δ 6.79 (dd, J=10.0, 8.6 Hz, 1H), 6.33 (dd, J=15.0, 2.5 Hz, 1H), 6.28 (dd, J=8.6, 2.7 Hz, 1H), 5.56 (t, J=5.8 Hz, 1H), 3.75-3.63 (m, 2H), 3.00-2.92 (m, 2H), 2.77 (t, J=6.2 Hz, 2H), 2.53-2.52 (m, 1H), 2.29-2.20 (m, 2H), 1.80-1.73 (m, 4H), 1.42-1.37 (m, 1H), 1.08 (d, J=6.2 Hz, 6H), 1.03-0.86 (m, 4H). MS (m/z): 336.3 [M+H]⁺.

### Compound 431

### N-(((1r,4r)-4-aminocyclohexyl)methyl)-4-(2,6-dimethylmorpholino)-2-fluoroaniline

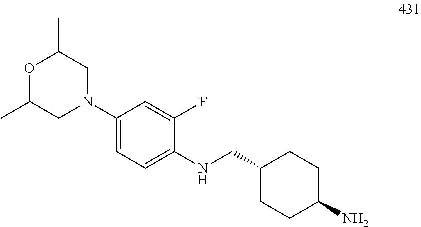 [SMILES: CC1CN(c2ccc(NC[C@H]3CC[C@H](N)CC3)c(F)c2)CC(C)O1]

The title compound 431 (20.9 mg, 27.1%) as a white solid was prepared tert-butyl ((1r,4r)-4-(((4-(2,6-dimethylmorpholino)-2-fluorophenyl)amino)methyl)cyclohexyl)carbamate (100 mg, 0.23 mmol), DCM (3 mL) and TFA (1 mL) according to the procedure for 24. ¹H NMR (400 MHZ, DMSO-d₆) δ 6.77-6.68 (m, 1H), 6.63-6.52 (m, 2H), 4.82-4.75 (m, 1H), 3.72-3.60 (m, 2H), 3.37-3.29 (m, 2H), 2.83 (t, J=6.4 Hz, 2H), 2.29-2.25 (m, 1H), 2.16-2.06 (m, 2H), 1.79-1.72 (m, 4H), 1.53-1.37 (m, 1H), 1.12 (d, J=6.3 Hz, 6H), 1.04-0.84 (m, 4H). MS (m/z): 336.3 [M+H]⁺.

### Compound 432

### N-(((1r,4r)-4-aminocyclohexyl)methyl)-4-(2,6-dimethylmorpholino)-5-fluoro-2-methylaniline

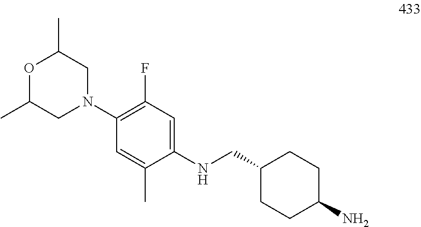 [SMILES: Cc1cc(N2CC(C)OC(C)C2)c(F)cc1NC[C@H]1CC[C@H](N)CC1]

The title compound 432 (41.2 mg) was prepared in a total yield of 66.1% as a pink solid from tert-butyl ((1r,4r)-4-(((4-(2,6-dimethylmorpholino)-5-fluoro-2-methylphenyl)amino)methyl)cyclohexyl)carbamate (80 mg, 0.178 mmol), TFA (1 mL), and DCM (10 mL) according to the procedure for 24. ¹H NMR (400 MHZ, DMSO-d₆) δ 8.40-8.22 (m, 3H), 6.71 (d, J=10.0 Hz, 1H), 6.28 (d, J=15.2 Hz, 1H), 4.83 (s, 1H), 3.76-3.63 (m, 2H), 2.98 (d, J=11.2 Hz, 2H), 2.87 (t, J=12.0 Hz, 3H), 2.27 (t, J=10.8 Hz, 2H), 2.06-1.93 (m, 5H), 1.89-1.80 (m, 2H), 1.52 (s, 1H), 1.34 (qd, J=12.4, 3.2 Hz, 2H), 1.09 (d, J=6.4 Hz, 6H), 1.03-0.92 (m, 2H). Mass (m/z): 350.3 [M+H]⁺.

### Compound 433

### N-(((1r,4r)-4-aminocyclohexyl)methyl)-4-(2,6-dimethylmorpholino)-3-fluoro-2-methylaniline

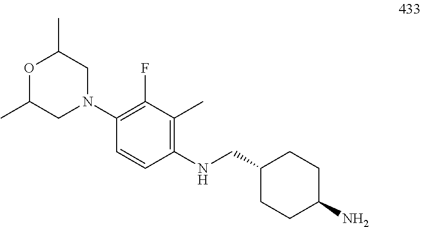 [SMILES: Cc1c(NC[C@H]2CC[C@H](N)CC2)ccc(N2CC(C)OC(C)C2)c1F]

The title compound 433 (23.5 mg) was prepared in a total yield of 43.1% as a green solid from tert-butyl ((1r,4r)-4-(((4-(2,6-dimethylmorpholino)-3-fluoro-2-methylphenyl)amino)methyl)cyclohexyl)carbamate (70 mg, 0.156 mmol), TFA (1 mL), and DCM (10 mL) according to the procedure for 24. ¹H NMR (400 MHz, DMSO-d₆) δ 8.32 (s, 3H), 6.70 (t, J=9.2 Hz, 1H), 6.23 (d, J=8.8 Hz, 1H), 4.84 (d, J=5.6 Hz, 1H), 3.77-3.64 (m, 2H), 3.03-2.95 (m, 2H), 2.86 (t, J=5.6 Hz, 3H), 2.23 (t, J=10.8 Hz, 2H), 2.05-1.93 (m, 5H), 1.90-1.80 (m, 2H), 1.53 (d, J=9.2 Hz, 1H), 1.35 (qd, J=12.4, 3.6 Hz, 2H), 1.09 (d, J=6.4 Hz, 6H), 1.04-0.91 (m, 2H). Mass (m/z): 350.3 [M+H]⁺.

### Compound 434

### N1-(2-(4-isopropylpiperidin-1-yl)pyrimidin-5-yl)bicyclo[1.1.1]pentane-1,3-diamine

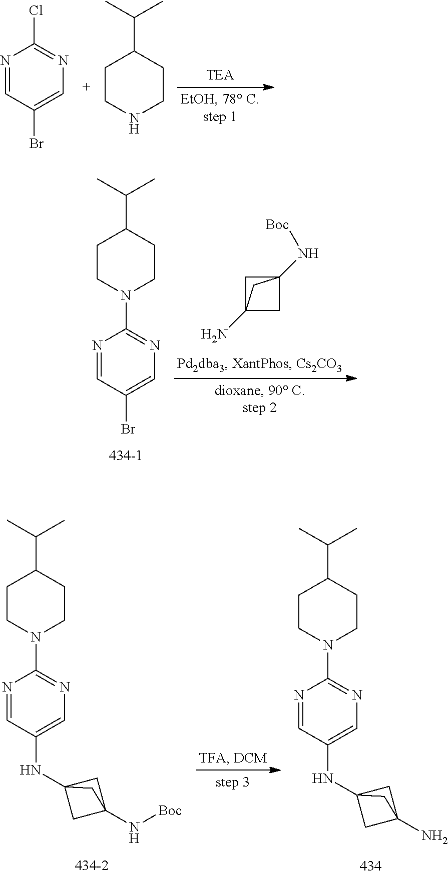

Step 1. Preparation of 5-bromo-2-(4-isopropylpiperidin-1-yl)pyrimidine (434-1) A mixture of 5-bromo-2-chloropyrimidine (200 mg, 1.04 mmol), trimethylamine (202 mg, 2.00 mmol) and 4-isopropylpiperidine (127 mg, 1.00 mmol) in ethanol (2 mL) was stirred under nitrogen at 78° C. overnight. The resulting mixture was concentrated under vacuum and purified by a fast silica gel column chromatography (petroleum ether/ethyl acetate 5/1) to give the desired product 5-bromo-2-(4-isopropylpiperidin-1-yl)pyrimidine (125 mg, 44.2%) as a white solid. MS (m/z): 284, 286 [M+H]⁺.

Step 2. Preparation of tert-butyl (3-((2-(4-isopropylpiperidin-1-yl)pyrimidin-5-yl)amino)bicyclo[1.1.1]pentan-1-yl)carbamate (434-2)

A mixture of 5-bromo-2-(4-isopropylpiperidin-1-yl)pyrimidine (110 mg, 0.41 mmol), tert-butyl (3-aminobicyclo[1.1.1]pentan-1-yl)carbamate (100 mg, 0.51 mmol), Cs₂CO₃ (200 mg, 0.62 mmol), Pd₂dba₃ (18 mg, 0.02 mmol) and XantPhos (40 mg, 0.07 mmol) in dioxane (3 mL) was bubbled with nitrogen for 3 min and then stirred overnight at 90° C. Additional tert-butyl (3-aminobicyclo[1.1.1]pentan-1-yl)carbamate (30 mg), Cs₂CO₃ (50 mg), Pd₂dba₃ (9 mg) and XantPhos (20 mg) were added and the mixture was stirred for 16 h. Starting materials still remained a little and the reaction was cooled and diluted with water. The resulting mixture was extracted with ethyl acetate twice and the combined organic layers were washed with brine, dried over sodium sulfate and concentrated under vacuum. The residue was purified by Prep-TLC (DCM/MeOH 20/1) to afford tert-butyl (3-((2-(4-isopropylpiperidin-1-yl)pyrimidin-5-yl)amino) bicyclo[1.1.1]pentan-1-yl)carbamate (46 mg, 28%) as a yellow solid. MS (m/z): 402 [M−H]⁺.

Step 3 Preparation of N1-(2-(4-isopropylpiperidin-1-yl)pyrimidin-5-yl)bicyclo[1.1.1]pentane-1,3-diamine (434) To a solution of tert-butyl (3-((2-(4-isopropylpiperidin-1-yl)pyrimidin-5-yl)amino) bicyclo[1.1.1]pentan-1-yl)carbamate (45 mg, 0.11 mmol) in dichloromethane (1 mL) was added trifluoroacetic acid (0.2 mL) and stirred overnight at room temperature. The resulting mixture was diluted with ethyl acetate and washed with sodium bicarbonate solution and brine. The organic layer was dried over sodium sulfate and concentrated under vacuum. The residue was purified by Prep-TLC (dichloromethane/methanol 15/1) to afford the title compound

N1-(2-(4-isopropylpiperidin-1-yl)pyrimidin-5-yl)bicyclo[1.1.1]pentane-1,3-diamine 434 (1.5 mg, 4.5%) as colorless oil. ¹H NMR (400 MHZ, Chloroform-d) δ 8.12 (s, 2H), 4.69 (d, J=13.1 Hz, 2H), 2.82-2.68 (m, 2H), 2.59 (s, 2H), 2.42 (s, 2H), 1.76-1.65 (m, 2H), 1.39 (dt, J=13.3, 6.7 Hz, 1H), 1.29-1.05 (m, 9H), 0.83 (d, J=6.7 Hz, 6H). MS (m/z): 302 [M+H]⁺.

### Compound 435

### 4-((5-fluoro-2-methyl-4-(4-(trifluoromethyl)piperidin-1-yl)phenyl)amino)cyclohexane-1-carboxamide

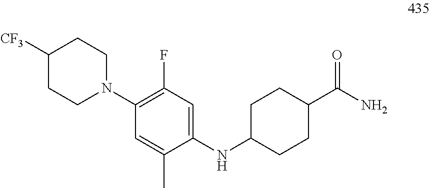 [SMILES: Cc1cc(N2CCC(C(F)(F)F)CC2)c(F)cc1NC1CCC(C(N)=O)CC1]

The title compound 435 (37.9 mg, 26.1%) was prepared from 5-fluoro-2-methyl-4-(4-(trifluoromethyl)piperidin-1-yl)aniline (100 mg, 0.36 mmol), 4-oxocyclohexane-1-carboxamide (50 mg, 0.36 mmol), NaBH(OAc)₃ (76.3 mg, 0.36 mmol), and DCE (10 mL) according to the procedure for 4. ¹H NMR (400 MHZ, DMSO-d₆) δ 7.15 (s, 1H), 6.71 (d, J=10.0 Hz, 2H), 6.30 (d, J=15.0 Hz, 1H), 4.11 (d, J=6.4 Hz, 1H), 3.33 (d, J=3.0 Hz, 1H), 3.13 (d, J=11.6 Hz, 2H), 2.58 (dd, J=11.8, 10.2 Hz, 2H), 2.39-2.28 (m, 1H), 2.18 (tt, J=7.8, 4.0 Hz, 1H), 2.00 (s, 3H), 1.82 (d, J=12.0 Hz, 2H), 1.74-1.54 (m, 10H). Mass (m/z): 401.8 [M+H]⁺.

### Compound 436

### N-(((1r, 4r)-4-aminocyclohexyl)methyl)-2-fluoro-6-(4-(trifluoromethyl)piperidin-1-yl)pyridin-3-amine

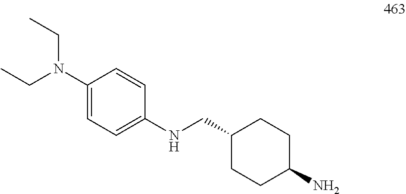 [SMILES: CCN(CC)c1ccc(NC[C@H]2CC[C@H](N)CC2)cc1]

The title compound 463 (14.1 mg, 22%) as a yellow solid was prepared from tert-butyl ((1r,4r)-4-(((4-(diethylamino)phenyl)amino)methyl)cyclohexyl)carbamate (190 mg, 0.5 mmol), DCM (5 mL) and TFA (1 mL) according to the procedure for 24. ¹H NMR (400 MHz, DMSO-d₆) δ=6.59 (d, J=8.8, 2H), 6.46 (d, J=8.8, 2H), 4.90 (s, 1H), 3.08 (q, J=7.2, 4H), 2.75 (d, J=4.4, 2H), 2.47-2.43 (m, 1H), 1.86-1.66 (m, 4H), 1.49-1.34 (m, 2H), 1.00-0.88 (m, 9H). Mass (m/z): 276.3 [M+H]⁺.

### Compound 437

### 3-((3-methyl-4-(4-(trifluoromethyl)piperidin-1-yl)phenyl)amino)cyclobutane-1-carboxamide

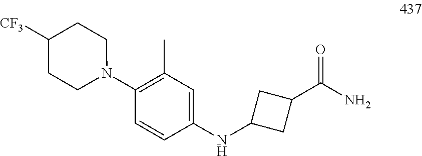 [SMILES: Cc1cc(NC2CC(C(N)=O)C2)ccc1N1CCC(C(F)(F)F)CC1]

The title compound 437 (27.5 mg, 27.5%) was prepared as a yellow solid from 3-({3-methyl-4-[4-(trifluoromethyl)piperidin-1-yl]phenyl}amino)cyclobutane-1-carboxylic acid (100 mg, 0.28 mmol), ammonium chloride (30 mg, 0.56 mmol), triethylamine (85 mg, 0.84 mmol), and HATU (160 mg, 0.42 mmol) according to the procedure for 1. ¹H NMR (400 MHz, DMSO-d₆) δ 7.17 (s, 1H), 6.77 (d, J=8.5 Hz, 1H), 6.71 (s, 1H), 6.27 (d, J=2.6 Hz, 1H), 6.21 (dd, J=8.5, 2.7 Hz, 1H), 5.48 (d, J=6.7 Hz, 1H), 3.81 (dd, J=13.9, 6.8 Hz, 1H), 2.89 (m, 3H), 2.51 (t, J=3.3 Hz, 1H), 2.42-2.36 (m, 3H), 2.31-2.28 (m, 1H), 2.10 (s, 3H), 1.95-1.89 (m, 2H), 1.82 (d, J=12.3 Hz, 2H), 1.54 (dd, J=12.3, 3.7 Hz, 2H). Mass (m/z): 356.3 [M+H]⁺.

### Compound 438

### N-(((1r,4r)-4-aminocyclohexyl)methyl)-3-chloro-4-(2,6-dimethylmorpholino)aniline

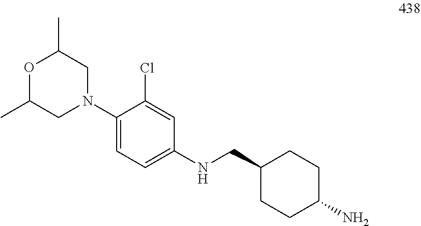 [SMILES: CC1CN(c2ccc(NC[C@H]3CC[C@H](N)CC3)cc2Cl)CC(C)O1]

The title compound 438 (41.0 mg) was prepared in a yield of 40.07% as a white powder from 3-chloro-4-(2,6-dimethylmorpholino) aniline (70 mg, 0.29 mmol) and tert-butyl ((1r,4r)-4-formylcyclohexyl)carbamate (99 mg, 0.44 mmol) according to the procedure for 20. 1¹H NMR (400 MHZ, DMSO-d₆) δ 8.04-7.84 (m, 3H), 6.92 (d, J=8.7 Hz, 1H), 6.60 (d, J=2.6 Hz, 1H), 6.48 (dd, J=8.7, 2.6 Hz, 1H), 5.70 (s, 1H), 3.69 (dqd, J=12.6, 6.2, 2.0 Hz, 2H), 3.00-2.88 (m, 3H), 2.81 (d, J=6.5 Hz, 2H), 2.26 (dd, J=11.4, 9.9 Hz, 2H), 1.98-1.80 (m, 4H), 1.44 (s, 1H), 1.31-1.20 (m, 2H), 1.09 (d, J=6.3 Hz, 6H), 1.00 (q, J=11.8 Hz, 2H). Mass (m/z): 352.5 [M+H]⁺.

### Compound 439

### N-(((1r,4r)-4-aminocyclohexyl)methyl)-4-(2,6-dimethylmorpholino)-3,5-difluoroaniline

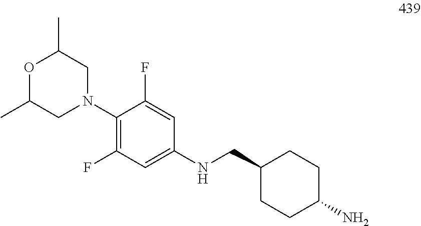 [SMILES: CC1CN(c2c(F)cc(NC[C@H]3CC[C@H](N)CC3)cc2F)CC(C)O1]

The title compound 439 (46.2 mg) was prepared in a yield of 31.67% as a white powder from 4-(2,6-dimethylmorpholino)-3,5-difluoroaniline (100 mg, 0.41 mmol) and tert-butyl ((1r,4r)-4-formylcyclohexyl)carbamate (175 mg, 0.83 mmol) according to the procedure for 20. 1¹H NMR (400 MHZ, DMSO-d₆) δ 7.31 (s, 2H), 6.22-5.98 (m, 3H), 3.61 (dtd, J=8.6, 6.1, 2.3 Hz, 2H), 2.96-2.86 (m, 1H), 2.84-2.72 (m, 4H), 2.68-2.61 (m, 2H), 2.01-1.77 (m, 4H), 1.42 (d, J=3.5 Hz, 1H), 1.36-1.22 (m, 2H), 1.04 (d, J=6.2 Hz, 6H), 1.01-0.91 (m, 2H). Mass (m/z): 354.5 [M+H]⁺.

### Compound 440

### N1-(4-(2,6-dimethylmorpholino)phenyl)cyclohexane-1,2-diamine

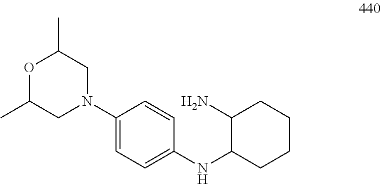 [SMILES: CC1CN(c2ccc(NC3CCCCC3N)cc2)CC(C)O1]

The title compound 440 (20.9 mg) was prepared in a yield of 20.9% as a white powder from 4-(2,6-dimethylmorpholino) aniline (64 mg, 0.31 mmol) and tert-butyl (2-oxocyclohexyl)carbamate (100 mg, 0.47 mmol) according to the procedure for 20. ¹H NMR (400 MHZ, DMSO-d₆) δ 7.92 (s, 2H), 6.82-6.75 (m, 2H), 6.67-6.57 (m, 2H), 4.90 (s, 1H), 3.67 (ddt, J=13.2, 6.9, 3.4 Hz, 2H), 3.33-3.28 (m, 2H), 3.15 (s, 1H), 2.88 (d, J=9.4 Hz, 1H), 2.12 (ddd, J=11.9, 10.3, 1.7 Hz, 2H), 2.00 (dd, J=34.1, 12.8 Hz, 2H), 1.68 (d, J=23.8 Hz, 2H), 1.41 (q, J=12.3, 11.8 Hz, 1H), 1.25 (q, J=11.1, 10.7 Hz, 2H), 1.12 (d, J=6.2 Hz, 6H), 1.10-1.00 (m, 1H). Mass (m/z): 304.5 [M+H]⁺.

### Compound 441

### N1-(4-(2,6-dimethylmorpholino)-2-fluorophenyl)cyclohexane-1,2-diamine

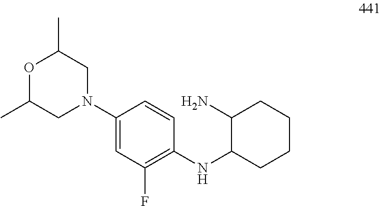 [SMILES: CC1CN(c2ccc(NC3CCCCC3N)c(F)c2)CC(C)O1]

The title compound 441 (10.1 mg) was prepared in a yield of 10.6% as a white powder from 4-(2,6-dimethylmorpholino)-2-fluoroaniline (71 mg, 0.31 mmol) and tert-butyl (2-oxocyclohexyl)carbamate (100 mg, 0.47 mmol) according to the procedure for 20. ¹H NMR (400 MHZ, DMSO-d₆) δ 7.89 (s, 2H), 6.84-6.71 (m, 2H), 6.63 (d, J=9.2 Hz, 1H), 4.67 (d, J=10.4 Hz, 1H), 3.72-3.59 (m, 2H), 3.39 (d, J=11.4 Hz, 2H), 3.18 (s, 1H), 3.05 (s, 1H), 2.13 (t, J=10.8 Hz, 2H), 2.04 (d, J=12.6 Hz, 1H), 1.90 (d, J=7.0 Hz, 1H), 1.68 (d, J=29.5 Hz, 2H), 1.40 (d, J=12.5 Hz, 1H), 1.28-1.17 (m, 2H), 1.13 (d, J=6.3 Hz, 6H). Mass (m/z): 322.3 [M+H]⁺.

### Compound 442

### N1-(4-(2,6-dimethylmorpholino)-2-methylphenyl)cyclohexane-1,2-diamine

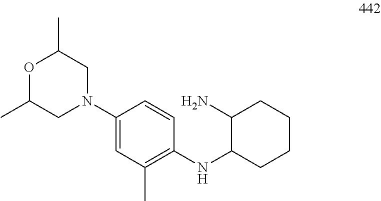 [SMILES: Cc1cc(N2CC(C)OC(C)C2)ccc1NC1CCCCC1N]

The title compound 442 (33.3 mg) was prepared in a yield of 34.5% as a white powder from 4-(2,6-dimethylmorpholino)-2-methylaniline (69 mg, 0.31 mmol) and tert-butyl (2-oxocyclohexyl)carbamate (100 mg, 0.47 mmol) according to the procedure for 20. ¹H NMR (400 MHZ, DMSO-d₆) δ 7.88 (s, 2H), 6.79-6.50 (m, 3H), 4.14 (s, 1H), 3.67 (s, 2H), 3.15 (d, J=37.6 Hz, 2H), 2.17 (s, 1H), 2.12 (s, 6H), 1.96 (d, J=12.6 Hz, 1H), 1.80-1.59 (m, 2H), 1.55-1.32 (m, 2H), 1.23 (s, 2H), 1.12 (d, J=6.2 Hz, 6H). Mass (m/z): 318.4 [M+H]⁺.

### Compound 443

### N1-(6-(2,6-dimethylmorpholino)-2-methylpyridin-3-yl)cyclohexane-1,4-diamine

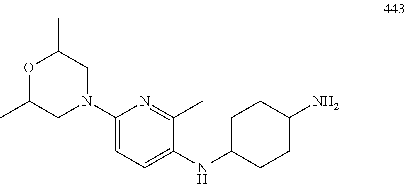 [SMILES: Cc1nc(N2CC(C)OC(C)C2)ccc1NC1CCC(N)CC1]

The title compound 443 (17.7 mg) was prepared in a yield of 24.6% as a white powder from 6-(2,6-dimethylmorpholino)-2-methylpyridin-3-amine (50 mg, 0.22 mmol) and tert-butyl (4-oxocyclohexyl)carbamate (72 mg, 0.34 mmol) according to the procedure for 20. ¹H NMR (400 MHZ, DMSO-d₆) δ 8.16 (d, J=21.0 Hz, 3H), 6.99 (s, 1H), 6.59 (s, 1H), 3.88 (d, J=11.7 Hz, 2H), 3.60 (d, J=8.6 Hz, 2H), 3.45 (s, 1H), 3.10 (s, 1H), 2.94 (s, 1H), 2.38-2.12 (m, 5H), 2.05-1.91 (m, 2H), 1.85-1.67 (m, 3H), 1.60 (s, 1H), 1.43 (d, J=12.2 Hz, 1H), 1.14 (d, J=6.2 Hz, 6H). Mass (m/z): 319.3 [M+H]⁺.

### Compound 444

### (1R, 2R)—N1-(4-(4-(trifluoromethyl)piperidin-1-yl)phenyl)cyclopentane-1,2-diamine

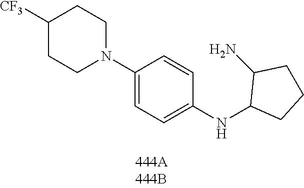 [SMILES: NC1CCCC1Nc1ccc(N2CCC(C(F)(F)F)CC2)cc1]

The title compound 444A (12 mg) was prepared in a yield of 17.9% as a white powder and compound 444B (23 mg) was prepared in a yield of 36% as a white powder from 4-(4-(trifluoromethyl)piperidin-1-yl)aniline (50 mg, 0.20 mmol) and tert-butyl (2-oxocyclopentyl)carbamate (61 mg, 0.30 mmol) according to the procedure for 20. 444A: ¹H NMR (400 MHZ, DMSO-d₆) δ 6.14 (d, J=8.3 Hz, 2H), 5.91 (d, J=8.1 Hz, 2H), 2.98 (dq, J=11.1, 6.3, 5.6 Hz, 2H), 2.69 (d, J=12.1 Hz, 2H), 1.85 (t, J=12.1 Hz, 2H), 1.52-1.37 (m, 2H), 1.37-1.28 (m, 1H), 1.22-1.12 (m, 2H), 1.05 (q, J=8.1 Hz, 2H), 0.99-0.82 (m, 4H). Mass (m/z): 328.3 [M+H]⁺. 444B: ¹H NMR (400 MHZ, DMSO-d₆) δ 6.12 (d, J=8.4 Hz, 2H), 5.86 (d, J=8.2 Hz, 2H), 2.93 (t, J=7.1 Hz, 1H), 2.68 (d, J=11.9 Hz, 2H), 2.58 (d, J=7.2 Hz, 1H), 1.85 (t, J=12.1 Hz, 2H), 1.41 (tt, J=13.9, 7.1 Hz, 3H), 1.16 (d, J=13.0 Hz, 2H), 1.04 (q, J=7.4 Hz, 2H), 0.91 (dp, J=21.7, 8.2 Hz, 3H), 0.69 (dd, J=13.7, 7.3 Hz, 1H). Mass (m/z): 328.3 [M+H]⁺.

### Compound 445

### N-(((1r,4r)-4-aminocyclohexyl)methyl)-4-(2,2,6,6-tetramethylmorpholino)aniline

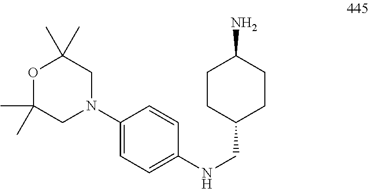 [SMILES: CC1(C)CN(c2ccc(NC[C@H]3CC[C@H](N)CC3)cc2)CC(C)(C)O1]

The title compound 445 (31.5 mg) was prepared in a total yield of 60.9% as a dark blue solid according to the procedure for compound 354. ¹H NMR (400 MHZ, Pyridine-d₅) δ 7.04-6.96 (m, 2H), 6.92-6.84 (m, 2H), 5.19 (s, 3H), 3.44-3.35 (m, 1H), 2.99-2.95 (m, 2H), 2.52-2.42 (m, 2H), 1.95-1.88 (m, 2H), 1.85-1.74 (m, 2H), 1.66-1.57 (m, 1H), 1.43-1.15 (m, 16H), 1.07-0.95 (m, 2H). Mass (m/z): 346.3 [M+H]⁺.

### Compound 446

### 4-((4-(4-methylpiperidin-1-yl)phenyl)amino)cyclohexane-1-carboxamide

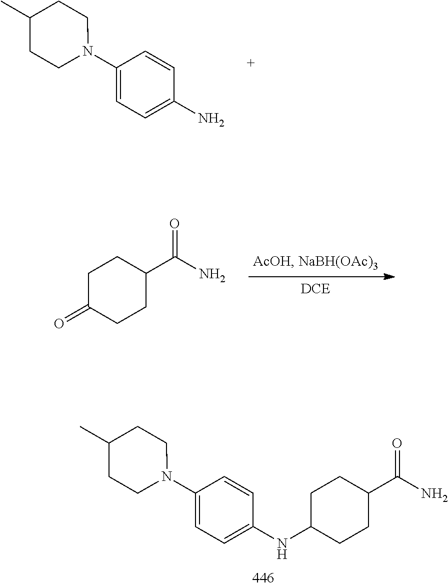 [SMILES: CC1CCN(c2ccc(N)cc2)CC1 ; NC(=O)C1CCC(=O)CC1 ; CC1CCN(c2ccc(NC3CCC(C(N)=O)CC3)cc2)CC1]

A solution of 4-(4-methylpiperidin-1-yl)aniline (50 mg, 0.240 mmol), 4-oxocyclohexane-1-carboxamide (51 mg, 0.360 mmol) and AcOH (0.1 mL) in DCE (10 mL) was stirred at r.t. for 30 min before NaBH (OAc) 3 (130 mg, 0.614 mmol) was added. The mixture was stirred at room temperature for 2 h. The mixture was extracted by DCM (25 mL×3). The combined organic layers were washed with brine (15 mL×3), dried over Na₂SO₄ and concentrated to give the crude product, which was purified by prep HPLC (XSelect-CSH-Prep 5 μm OBD, 19*150 mm column; ACN/water (0.5% NH₃H₂O)=50%-77%-95%-95%-0%, 0 min-8 min-8.5 min-9.5 min-11 min), to give the desired product 446A (Rt=6.1 min) as white solid (24.6 mg, 31.1%) and 446B (Rt=7.1 min) as white solid (27.1 mg, 33.3%). 446A H NMR (400 MHZ, DMSO-d₆) δ 7.23-7.17 (m, 1H), 6.77 (d, J=8.4 Hz, 1H), 6.69-6.63 (m, 1H), 6.38 (d, J=2.8 Hz, 1H), 6.32 (dd, J=8.4, 2.8 Hz, 1H), 4.89 (d, J=8.4 Hz, 1H), 3.04 (tdt, J=11.2, 7.2, 3.6 Hz, 1H), 2.83 (dt, J=12.0, 3.2 Hz, 2H), 2.46 (dd, J=11.6, 2.4 Hz, 2H), 2.11 (s, 3H), 2.01 (ddq, J=27.2, 12.0, 3.6 Hz, 3H), 1.76 (dt, J=14.4, 3.6 Hz, 2H), 1.69-1.60 (m, 2H), 1.43 (qd, J=13.0, 3.6 Hz, 3H), 1.25 (qd, J=12.0, 3.6 Hz, 2H), 1.13-0.99 (m, 2H), 0.94 (d, J=6.4 Hz, 3H). 446B ¹H NMR (400 MHZ, DMSO-d₆) δ 7.16 (s, 1H), 6.78 (d, J=8.4 Hz, 1H), 6.69 (s, 1H), 6.43 (d, J=2.8 Hz, 1H), 6.36 (dd, J=8.4, 2.8 Hz, 1H), 5.00 (d, J=7.6 Hz, 1H), 2.83 (dt, J=12.0, 3.6 Hz, 2H), 2.46 (dd, J=11.6, 2.4 Hz, 2H), 2.12 (s, 4H), 1.83-1.71 (m, 2H), 1.70-1.60 (m, 4H), 1.59-1.35 (m, 5H), 1.25 (qd, J=11.6, 3.6 Hz, 2H), 0.94 (d, J=6.4 Hz, 3H). Mass (m/z): 330.3 [M+H]⁺.

### Compound 447

### N-(4-((1r,4s)-4-aminocyclohexyl)butyl)-2-methyl-4-(4-(trifluoromethyl)piperidin-1-yl)aniline

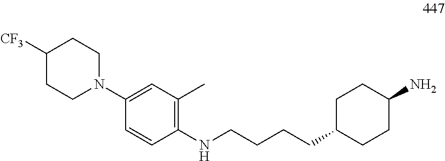 [SMILES: Cc1cc(N2CCC(C(F)(F)F)CC2)ccc1NCCCC[C@H]1CC[C@H](N)CC1]

The title compound 447 (17.4 mg, 23.2%) as a white solid was prepared from 4-((1r,4s)-4-aminocyclohexyl)-N-(2-methyl-4-(4-(trifluoromethyl)piperidin-1-yl)phenyl)butana mide (100 mg, 0.18 mmol) in BH₃-THF (20 mL) according to the procedure for 105. ¹H NMR (400 MHZ, CD₃OD) δ 6.80 (s, 2H), 6.58 (d, J=8.7 Hz, 1H), 3.47 (d, J=11.8 Hz, 2H), 3.10 (dd, J=14.9, 7.8 Hz, 2H), 2.86-2.77 (m, 1H), 2.60 (t, J=11.2 Hz, 2H), 2.31-2.22 (m, 1H), 2.12 (s, 3H), 1.95 (d, J=11.9 Hz, 4H), 1.84 (d, J=12.1 Hz, 2H), 1.78-1.68 (m, 2H), 1.61 (dt, J=15.0, 7.5 Hz, 2H), 1.41 (d, J=15.5 Hz, 2H), 1.32-1.19 (m, 6H), 1.07-0.95 (m, 2H). Mass (m/z): 412.3 [M+H]⁺.

### Compound 448

### N-(((1r,3r)-3-aminocyclobutyl)methyl)-2-(4-isopropylpiperidin-1-yl)pyrimidin-5-amine

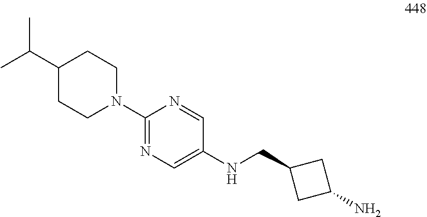 [SMILES: CC(C)C1CCN(c2ncc(NC[C@H]3C[C@H](N)C3)cn2)CC1]

The title compound 448 (28.2 mg) as a yellow solid was prepared from tert-butyl ((1r,3r)-3-(((2-(4-isopropylpiperidin-1-yl)pyrimidin-5-yl)amino)methyl)cyclobutyl)carbamate (100 mg, 0.24 mmol), 1,4-dioxane (3 mL) and HCl/1,4-dioxane (1 mL) according to the procedure for 37. ¹H NMR (400 MHZ, CD₃OD) δ 7.89 (s, 2H), 4.47 (d, J=13.2 Hz, 2H), 3.83 (d, J=7.4 Hz, 1H), 3.12 (d, J=7.4 Hz, 2H), 2.70 (s, 2H), 2.25 (d, J=5.6 Hz, 4H), 1.70 (d, J=11.6 Hz, 2H), 1.41 (qd, J=13.2, 6.6 Hz, 1H), 1.22-1.13 (m, 4H), 0.88 (d, J=6.8 Hz, 6H). Mass (m/z): 304.3 [M+H]⁺.

### Compound 449

### 4-(4-(((4-aminocyclohexyl)methyl)amino)phenyl) thiomorpholine 1,1-dioxide

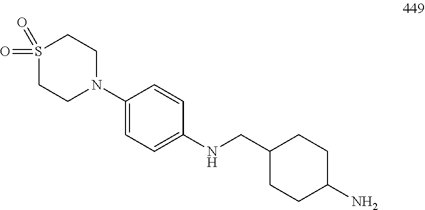 [SMILES: NC1CCC(CNc2ccc(N3CCS(=O)(=O)CC3)cc2)CC1]

The title compound 449 (122 mg, 66.7%) as a yellow solid was prepared from tert-butyl (4-(((4-(1,1-dioxidothiomorpholino phenyl)amino)methyl)cyclohexyl)carbamate (200 mg, 0.46 mmol), DCM (5 mL) and TFA (1 mL) according to the procedure for 24. ¹H NMR (400 MHZ, CDCl₃) δ 6.83 (d, J=8.4, 2H), 6.55 (d, J=8.4, 2H), 3.58 (s, 4H), 3.11 (s, 4H), 2.92 (d, J=6.4, 2H), 2.62 (s, 1H), 1.90-1.83 (m, 4H), 1.51 (s, 1H), 1.38 (s, 1H), 1.20-0.94 (m, 4H). Mass (m/z): 337.9 [M+H]⁺.

### Compound 450

### N1-(4-(2,6-dimethylmorpholino)phenyl)cyclohexane-1,4-diamine

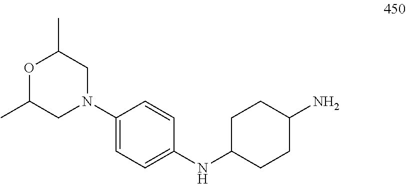 [SMILES: CC1CN(c2ccc(NC3CCC(N)CC3)cc2)CC(C)O1]

The desired product 450 as a yellow solid (21.5 mg, 24.4%) was prepared from tert-butyl (4-((4-(2,6-dimethylmorpholino)phenyl)amino)cyclohexyl)carbamate according to the procedure for 24. ¹H NMR (400 MHZ, DMSO-d₆) δ 7.93 (s, 1H), 7.07 (d, J=8.5 Hz, 2H), 6.94 (t, J=10.7 Hz, 2H), 3.5-3.2 (m, 6H), 1.6-1.5 (m, 2H), 1.20 (d, J=4.9 Hz, 6H), 0.71-0.64 (m, 2H). Mass (m/z): 304.2 [M+H]⁺

### Compound 451

### (((1r,4r)-4-aminocyclohexyl)methyl)-4-(2,6-dimethylmorpholino)-3,5-dimethylaniline

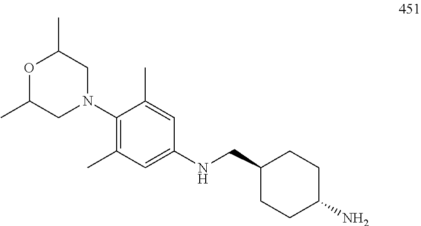 [SMILES: Cc1cc(NC[C@H]2CC[C@H](N)CC2)cc(C)c1N1CC(C)OC(C)C1]

The title compound 451 (14.2 mg) was prepared in a total yield of 41.0% as a white solid according to the procedure for compound 24. ¹H NMR (400 MHz, DMSO-d₆) δ 6.20 (d, J=2.8 Hz, 1H), 6.08 (d, J=2.8 Hz, 1H), 3.73-3.58 (m, 2H), 2.90-2.72 (m, 5H), 2.65-2.57 (m, 2H), 2.14 (s, 3H), 2.12 (s, 3H), 2.02-1.75 (m, 4H), 1.64-1.42 (m, 2H), 1.36-1.23 (m, 3H), 1.05 (d, J=6.4 Hz, 6H), 1.00-0.87 (m, 2H). Mass (m/z): 346.3 [M+H]⁺

### Compound 452

### N-(((1r,4r)-4-aminocyclohexyl)methyl)-4-(2,6-dimethylmorpholino)-2,3-dimethylaniline

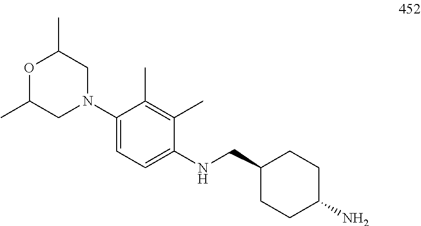 [SMILES: Cc1c(NC[C@H]2CC[C@H](N)CC2)ccc(N2CC(C)OC(C)C2)c1C]

The title compound 452 (22.5 mg) was prepared in a total yield of 65.2% as a white solid according to the procedure for compound 24. ¹H NMR (400 MHZ, DMSO-d₆) δ 6.21 (d, J=2.8 Hz, 1H), 6.09 (d, J=2.8 Hz, 1H), 3.72-3.61 (m, 2H), 2.95-2.74 (m, 5H), 2.63-2.56 (m, 2H), 2.14 (s, 3H), 2.13 (s, 3H), 2.02-1.92 (m, 2H), 1.88-1.80 (m, 2H), 1.36-1.22 (m, 3H), 1.06 (d, J=6.4 Hz, 6H), 1.02-0.88 (m, 2H). Mass (m/z): 346.3 [M+H]⁺

### Compound 453

### N-(((1r,4r)-4-aminocyclohexyl)methyl)-4-(2,6-dimethylmorpholino)-2,5-dimethylaniline

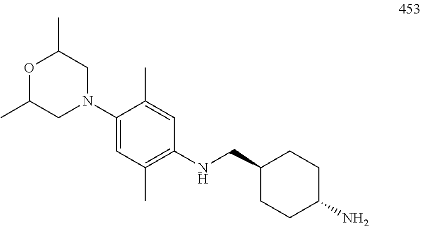 [SMILES: Cc1cc(N2CC(C)OC(C)C2)c(C)cc1NC[C@H]1CC[C@H](N)CC1]

The title compound 453 (18.1 mg) was prepared in a total yield of 52.7% as a white solid according to the procedure for compound 24. ¹H NMR (400 MHZ, DMSO-d₆) δ 6.69 (s, 1H), 6.30 (s, 1H), 3.76-3.62 (m, 2H), 3.01-2.83 (m, 3H), 2.78-2.67 (m, 2H), 2.35-2.21 (m, 2H), 2.17 (s, 3H), 2.02 (s, 3H), 2.00-1.91 (m, 2H), 1.91-1.82 (m, 2H), 1.39-1.17 (m, 3H), 1.08 (d, J=6.4 Hz, 6H), 1.01-0.89 (m, 2H). Mass (m/z): 346.3 [M+H]⁺.

### Compound 454

### (1R, 2S)-N1-(4-(4-(trifluoromethyl)piperidin-1-yl)phenyl)cyclopentane-1.2-diamine

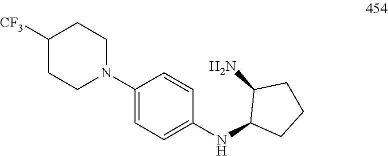 [SMILES: N[C@H]1CCC[C@H]1Nc1ccc(N2CCC(C(F)(F)F)CC2)cc1]

The title compound 454 (23 mg) was prepared in a yield of 36% as a white powder from 4-(4-(trifluoromethyl)piperidin-1-yl)aniline (50 mg, 0.20 mmol) and tert-butyl (2-oxocyclopentyl)carbamate (61 mg, 0.30 mmol) according to the procedure for 24. ¹H NMR (400 MHZ, DMSO-d₆) δ 6.12 (d, J=8.4 Hz, 2H), 5.86 (d, J=8.2 Hz, 2H), 2.93 (t, J=7.1 Hz, 1H), 2.68 (d, J=11.9 Hz, 2H), 2.58 (d, J=7.2 Hz, 1H), 1.85 (t, J=12.1 Hz, 2H), 1.41 (tt, J=13.9, 7.1 Hz, 3H), 1.16 (d, J=13.0 Hz, 2H), 1.04 (q, J=7.4 Hz, 2H), 0.91 (dp, J=21.7, 8.2 Hz, 3H), 0.69 (dd, J=13.7, 7.3 Hz, 1H). Mass (m/z): 328.3 [M+H]⁺.

### Compound 455

### (1s,4s)-N1-(6-(2,6-dimethylmorpholino)-2-methylpyridin-3-yl)cyclohexane-1,4-diamine

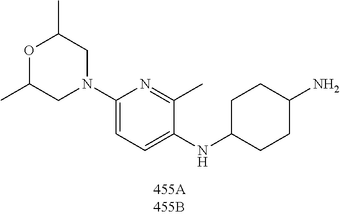 [SMILES: Cc1nc(N2CC(C)OC(C)C2)ccc1NC1CCC(N)CC1]

The title compound 455A (4.6 mg) was prepared in a yield of 6.5% as a white powder and compound 455B (1.4 mg) was prepared in a yield of 1.7% as a white powder from 6-(2,6-dimethylmorpholino)-2-methylpyridin-3-amine (Rt-7.62 min; 50 mg, 0.22 mmol) and tert-butyl (4-oxocyclohexyl)carbamate (Rt=8.07; 72 mg, 0.34 mmol) according to the procedure for 20, which were prepared by Prep-HPLC (Column: X Select-CSH-Prep 5 μm OBD, 19*150 mm; ACN/water (0.5% TFA)=0%-20%-95%-95%-10%, 0 min-10 min-10.5 min-11.5 min-13.0 min). 455A: ¹H NMR (400 MHZ, DMSO-d₆) δ 6.89 (d, J=8.9 Hz, 1H), 6.50 (d, J=8.7 Hz, 1H), 3.88 (d, J=8.3 Hz, 1H), 3.85-3.79 (m, 2H), 3.60 (ddd, J=10.4, 6.2, 2.4 Hz, 2H), 2.98 (s, 1H), 2.18 (s, 2H), 2.14 (dd, J=12.3, 10.4 Hz, 2H), 1.82 (dd, J=46.2, 11.3 Hz, 4H), 1.25-1.15 (m, 4H), 1.13 (d, J=6.2 Hz, 6H). Mass (m/z): 319.3 [M+H]⁺. 455B: ¹H NMR (400 MHZ, DMSO-d₆) δ 6.85 (d, J=8.9 Hz, 1H), 6.57 (d, J=8.7 Hz, 1H), 3.85 (d, J=8.3 Hz, 1H), 3.85-3.75 (m, 2H), 3.61 (ddd, J=10.4, 6.3, 2.2 Hz, 2H), 2.96 (s, 1H), 2.18 (s, 2H), 2.14 (dd, J=12.3, 10.4 Hz, 2H), 1.82 (dd, J=46.2, 11.3 Hz, 4H), 1.23-1.11 (m, 4H), 0.94 (d, J=6.5 Hz, 6H). Mass (m/z): 319.3 [M+H]⁺.

### Compound 456

### N1-(4-(2,6-dimethylmorpholino)-3-fluorophenyl)cyclohexane-1,4-diamine

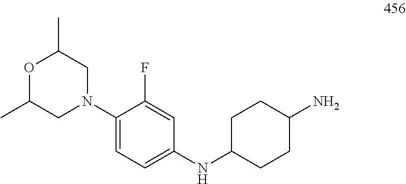 [SMILES: CC1CN(c2ccc(NC3CCC(N)CC3)cc2F)CC(C)O1]

The title compound 456 (24.3 mg) was prepared in a yield of 33.9% as a white powder from 4-(2,6-dimethylmorpholino)-3-fluoroaniline (50 mg, 0.22 mmol) and tert-butyl (4-oxocyclohexyl)carbamate (71 mg, 0.33 mmol) according to the procedure for 20. ¹H NMR (400 MHZ, DMSO-d₆) δ 8.18 (s, 2H), 6.80 (s, 1H), 6.48-6.27 (m, 2H), 5.43 (s, 1H), 3.69 (ddd, J=8.6, 6.4, 2.1 Hz, 2H), 3.07 (s, 1H), 2.97 (d, J=11.1 Hz, 3H), 2.25 (t, J=10.7 Hz, 2H), 1.98 (d, J=11.4 Hz, 4H), 1.73 (s, 1H), 1.59 (s, 1H), 1.45 (q, J=12.0 Hz, 2H), 1.09 (d, J=6.2 Hz, 6H). Mass (m/z): 322.5 [M+H]⁺.

### Compound 457

### N2-(4-(2,6-dimethylmorpholino)-3-fluorophenyl)spiro[3.3]heptane-2,6-diamine

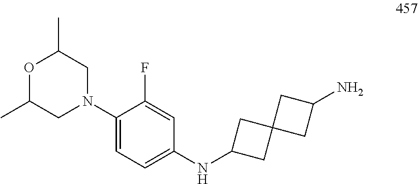 [SMILES: CC1CN(c2ccc(NC3CC4(CC(N)C4)C3)cc2F)CC(C)O1]

The title compound 457 (22.8 mg) was prepared in a yield of 30.7% as a white powder from 4-(2,6-dimethylmorpholino)-3-fluoroaniline (50 mg, 0.22 mmol) and tert-butyl (6-oxospiro [3.3]heptan-2-yl)carbamate (75 mg, 0.34 mmol) according to the procedure for 20. ¹H NMR (400 MHZ, DMSO-d₆) δ 8.19 (s, 2H), 6.80 (t, J=9.2 Hz, 1H), 6.33-6.17 (m, 2H), 5.83 (s, 1H), 3.67 (ddd, J=20.7, 11.2, 6.1 Hz, 3H), 3.54 (d, J=7.0 Hz, 1H), 2.97 (d, J=11.1 Hz, 2H), 2.43-2.29 (m, 3H), 2.25 (t, J=10.7 Hz, 2H), 2.21-2.10 (m, 3H), 1.86-1.73 (m, 2H), 1.08 (d, J=6.2 Hz, 6H). Mass (m/z): 334.6 [M+H]⁺.

### Compound 458

### N1-(4-(2,6-dimethylmorpholino)-3-fluorophenyl)-4-methylcyclohexane-1,4-diamine

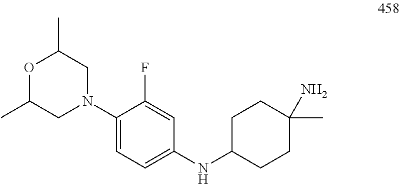

The title compound 458 (51.7 mg) was prepared in a yield of 69.1% as a white powder from 4-(2,6-dimethylmorpholino)-3-fluoroaniline (50 mg, 0.22 mmol) and tert-butyl (1-methyl-4-oxocyclohexyl)carbamate (61 mg, 0.27 mmol) according to the procedure for 20. ¹H NMR (400 MHZ, DMSO-d₆) δ 8.25 (d, J=32.3 Hz, 2H), 6.80 (t, J=9.3 Hz, 1H), 6.50-6.25 (m, 2H), 5.39 (d, J=50.6 Hz, 1H), 3.69 (ddt, J=13.9, 7.5, 3.8 Hz, 2H), 3.15 (d, J=42.1 Hz, 1H), 2.97 (d, J=11.0 Hz, 2H), 2.25 (t, J=10.7 Hz, 2H), 1.98-1.83 (m, 2H), 1.71 (p, J=12.0, 11.1 Hz, 3H), 1.63-1.47 (m, 2H), 1.28 (d, J=6.8 Hz, 4H), 1.08 (d, J=6.2 Hz, 6H). Mass (m/z): 336.3 [M+H]⁺.

### Compound 459

### N-(((1r,4r)-4-aminocyclohexyl)methyl)-4-(4-methoxy-4-(trifluoromethyl)piperidin-1-yl)aniline

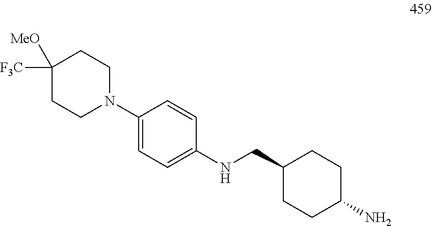 [SMILES: COC1(C(F)(F)F)CCN(c2ccc(NC[C@H]3CC[C@H](N)CC3)cc2)CC1]

The title compound 459 (42.3 mg) was prepared in a yield of 60.2% as a white powder from 4-(4-methoxy-4-(trifluoromethyl)piperidin-1-yl)aniline (50 mg, 0.18 mmol) and tert-butyl ((1r,4r)-4-formylcyclohexyl)carbamate (62 mg, 0.27 mmol) according to the procedure for 20. 1¹H NMR (400 MHZ, DMSO-d₆) δ 7.99 (s, 2H), 6.77 (d, J=8.4 Hz, 2H), 6.47 (d, J=8.4 Hz, 2H), 5.21 (s, 1H), 3.37 (d, J=1.5 Hz, 3H), 3.25 (d, J=12.0 Hz, 2H), 2.91 (s, 1H), 2.78 (s, 2H), 2.71-2.63 (m, 2H), 1.96 (d, J=12.7 Hz, 4H), 1.82 (q, J=12.0, 10.9 Hz, 4H), 1.44 (s, 1H), 1.27 (q, J=13.2, 12.8 Hz, 2H), 0.98 (q, J=12.5 Hz, 2H). Mass (m/z): 386.8 [M+H]⁺.

### Compound 460

### (3aR, 6aS)-N2-(4-(2,6-dimethylmorpholino)-3-fluorophenyl) octahydropentalene-2,5-diamine

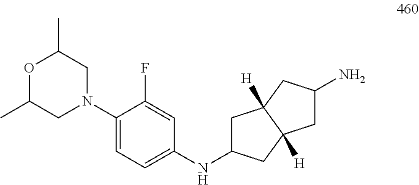 [SMILES: CC1CN(c2ccc(NC3C[C@H]4CC(N)C[C@H]4C3)cc2F)CC(C)O1]

The title compound 460 (19.7 mg) was prepared in a three-step total yield of 24.5% as a white solid with 1:0.1 mixture by ¹H NMR from 4-(2,6-dimethylmorpholino)-3-fluoroaniline (100 mg, 0.45 mmol) and (3as,6as)-tetrahydropentalene-2,5 (1H,3H)-dione (198 mg, 0.86 mmol) according to the procedure for 20. ¹H NMR (400 MHZ, Methanol-d₄) δ 7.53-7.38 (m, 2H), 6.85 (t, J=9.2 Hz, 1H), 6.41 (d, J=15.0 Hz, 2H), 4.11 (s, 1H), 3.81 (p, J=6.9, 5.5 Hz, 2H), 3.70 (dt, J=16.0, 6.4 Hz, 1H), 3.49 (tt, J=11.2, 6.5 Hz, 1H), 3.06 (d, J=11.3 Hz, 2H), 2.67 (d, J=10.3 Hz, 1H), 2.61-2.50 (m, 2H), 2.43-2.28 (m, 5H), 1.98-1.73 (m, 2H), 1.46 (td, J=11.9, 8.2 Hz, 1H), 1.17 (d, J=6.3 Hz, 6H). Mass (m/z): 347.3 [M+H]⁺.

### Compound 461

### N2-(4-(2,6-dimethylmorpholino)-2-fluorophenyl)spiro[3.3]heptane-2,6-diamine

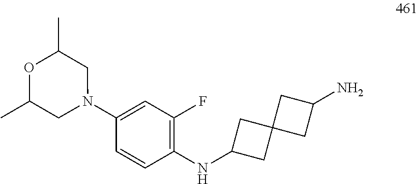 [SMILES: CC1CN(c2ccc(NC3CC4(CC(N)C4)C3)c(F)c2)CC(C)O1]

The title compound 461 (7.2 mg) was prepared in a total yield of 19.1% as a purple solid from tert-butyl (6-((4-(2,6-dimethylmorpholino)-2-fluorophenyl)amino)spiro[3.3]heptan-2-yl)carbamate (49 mg, 0.113 mmol), TFA (1 mL), and DCM (10 mL) according to the procedure for 24. ¹H NMR (400 MHZ, DMSO-d₆) δ 8.38 (s, 2H), 6.72 (dd, J=14.8, 2.4 Hz, 1H), 6.59-6.45 (m, 2H), 4.98 (d, J=7.2 Hz, 1H), 3.73-3.60 (m, 3H), 3.51 (q, J=8.0 Hz, 1H), 3.34 (dt, J=10.8, 2.0 Hz, 2H), 2.46 (td, J=6.8, 3.2 Hz, 1H), 2.34 (dddd, J=22.8, 11.6, 7.2, 3.6 Hz, 2H), 2.26-2.15 (m, 3H), 2.11 (dd, J=11.6, 10.0 Hz, 2H), 1.91 (dt, J=11.2, 7.2 Hz, 2H), 1.12 (d, J=6.4 Hz, 6H). Mass (m/z): 334.3 [M+H]⁺.

### Compound 462

### N1-(4-(2,6-dimethylmorpholino)-2-fluorophenyl)cyclobutane-1,3-diamine

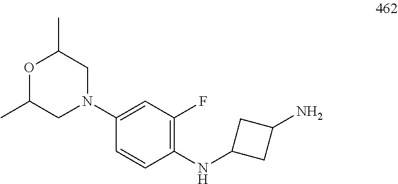 [SMILES: CC1CN(c2ccc(NC3CC(N)C3)c(F)c2)CC(C)O1]

The title compound 462 (7.8 mg) was prepared in a total yield of 20.9% as a green solid from tert-butyl (3-((4-(2,6-dimethylmorpholino)-2-fluorophenyl)amino)cyclobutyl)carbamate (50 mg, 0.127 mmol), TFA (1 mL), and DCM (10 mL) according to the procedure for 24. H NMR (400 MHZ, DMSO-d₆) δ 10.96 (s, 1H), 9.31-9.15 (m, 2H), 8.19-8.01 (m, 1H), 7.15 (t, J=9.2 Hz, 1H), 7.01-6.91 (m, 1H), 6.86 (dd, J=8.8, 2.8 Hz, 1H), 5.23 (dd, J=12.0, 1.6 Hz, 1H), 3.73-3.60 (m, 4H), 2.46 (s, 3H), 2.31 (dd, J=12.0, 10.0 Hz, 2H), 1.16 (d, J=6.0 Hz, 6H). Mass (m/z): 294.3 [M+H]⁺.

### Compound 463

### N2-(4-(2,6-dimethylmorpholino)-2-methylphenyl)spiro[3.3]heptane-2,6-diamine

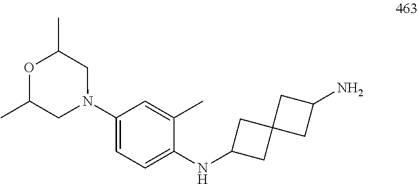 [SMILES: Cc1cc(N2CC(C)OC(C)C2)ccc1NC1CC2(CC(N)C2)C1]

The title compound 463 (33.8 mg) was prepared in a total yield of 50.0% as a purple solid from tert-butyl (6-((4-(2,6-dimethylmorpholino)-2-methylphenyl)amino)spiro[3.3]heptan-2-yl)carbamate (88 mg, 0.205 mmol), TFA (1 mL) and DCM (10 mL) according to the procedure for 24. ¹H NMR (400 MHZ, DMSO-d₆) δ 8.36 (s, 3H), 6.62 (d, J=2.8 Hz, 1H), 6.54 (dd, J=8.8, 2.8 Hz, 1H), 6.26 (d, J=8.8 Hz, 1H), 4.41 (d, J=6.8 Hz, 1H), 3.62 (dqd, J=10.4, 6.0, 2.4 Hz, 3H), 3.51-3.44 (m, 1H), 3.23 (dt, J=10.4, 2.0 Hz, 2H), 2.32 (dtd, J=16.8, 7.2, 4.0 Hz, 2H), 2.19 (dd, J=11.2, 8.4 Hz, 1H), 2.13 (dd, J=8.0, 4.0 Hz, 2H), 2.09-2.02 (m, 2H), 2.00 (s, 3H), 1.86 (ddd, J=11.2, 8.0, 5.6 Hz, 2H), 1.07 (d, J=6.0 Hz, 6H). Mass (m/z): 330.3 [M+H]⁺.

### Compound 464

### N-(((1r,4r)-4-aminocyclohexyl)methyl)-4-(2,6-dimethylmorpholino)-3-fluoro-5-methylaniline

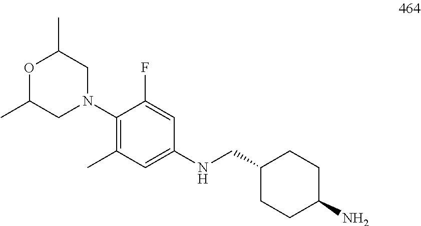 [SMILES: Cc1cc(NC[C@H]2CC[C@H](N)CC2)cc(F)c1N1CC(C)OC(C)C1]

The title compound 464 (15.2 mg) was prepared in a total yield of 7.1% as a light-yellow solid according to the procedure for compound 354. ¹H NMR (400 MHZ, Methanol-d₄) δ 6.26-6.21 (m, 1H), 6.15-6.07 (m, 1H), 3.79-3.71 (m, 2H), 3.08-3.00 (m, 1H), 2.93-2.87 (m, 2H), 2.82-2.75 (m, 2H), 2.69-2.64 (m, 2H), 2.21 (s, 3H), 2.10-1.94 (m, 4H), 1.65-1.57 (m, 1H), 1.43-1.33 (m, 2H), 1.18-1.02 (m, 8H). Mass (m/z): 350.2 [M+H]⁺.

### Compound 465

### N2-(2-methyl-4-(1,4-oxazepan-4-yl)phenyl)spiro[3.3]heptane-2,6-diamine

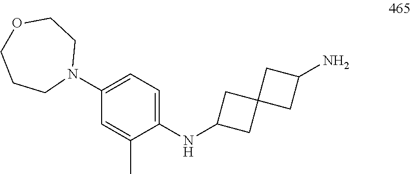 [SMILES: Cc1cc(N2CCCOCC2)ccc1NC1CC2(CC(N)C2)C1]

The desired product 465 (45 mg, 31.4%) as oil was prepared from tert-butyl (6-((2-methyl-4-(1,4-oxazepan-4-yl)phenyl)amino)spiro[3.3]heptan-2-yl)carbamate (180 mg, 0.4321 mmol), 1,4-dioxane (10 mL) and HCl/1,4-dioxane (10 mL) according to the procedure for 37. ¹H NMR (400 MHZ, DMSO-d₆) § 6.49 (s, 1H), 6.42 (d, J=8.4 Hz, 1H), 6.28 (d, J=8.6 Hz, 1H), 4.14 (brs, 1H), 3.72-3.65 (m, 2H), 3.59 (s, 1H), 3.54 (t, J=5.5 Hz, 2H), 3.38-3.24 (m, 2H), 3.19-3.13 (m, 1H), 2.43-2.37 (m, 1H), 2.33-2.23 (m, 2H), 2.14-2.09 (m, 1H), 2.03 (s, 3H), 1.83 (m, 5H), 1.69-1.59 (m, 2H). Mass (m/z): 316.23 [M+H]⁺.

### Compound 466

### N-(((1r,4r)-4-aminocyclohexyl)methyl)-4-(2,6-dimethylmorpholino)-2-(trifluoromethyl)aniline

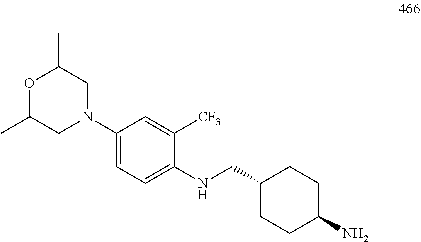 [SMILES: CC1CN(c2ccc(NC[C@H]3CC[C@H](N)CC3)c(C(F)(F)F)c2)CC(C)O1]

The title compound 466 (16.2 mg, 16.7%) as a yellow solid was prepared from tert-butyl ((1r,4r)-4-(((4-(2,6-dimethylmorpholino)-2-(trifluoromethyl)phenyl)amino)methyl)cyclohexyl) carbamate (120 mg, 0.2461 mmol), DCM (10 mL) and TFA (1 mL) according to the procedure for 24. ¹H NMR (400 MHZ, DMSO-d₆) δ 7.09 (d, J=9.1 Hz, 1H), 6.95 (d, J=2.5 Hz, 1H), 6.75 (d, J=9.0 Hz, 1H), 3.73-3.62 (m, 3H), 3.36 (brs, 7H), 2.96 (d, J=6.2 Hz, 2H), 2.85 (s, 1H), 2.14 (s, 1H), 1.89 (s, 2H), 1.78 (d, J=11.0 Hz, 2H), 1.13 (d, J=6.2 Hz, 6H). Mass (m/z): 386.23 [M+H]⁺.

### Compound 467

### N-(((1r,4r)-4-aminocyclohexyl)methyl)-4-(3,5-dimethyl-4-(2,2,2-trifluoroethyl) piperazin-1-yl)aniline

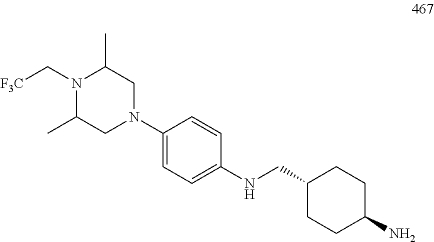 [SMILES: CC1CN(c2ccc(NC[C@H]3CC[C@H](N)CC3)cc2)CC(C)N1CC(F)(F)F]

The title compound 467 as a yellow solid (10 mg, 20%) was prepared from tert-butyl ((1,4r)-4-(((4-(3,5-dimethyl-4-(2,2,2-trifluoroethyl) piperazin-1-yl)phenyl)amino)methyl)cyclo hexyl)carbamate (0.06 g, 0.3 mmol), DCM (5 mL) and TFA (1 mL) according to the procedure for 24. ¹H NMR (400 MHZ, CD₃OD) δ 7.28 (d, J=9.0 Hz, 2H), 6.96 (d, J=9.0 Hz, 2H), 3.54-3.52 (m, 4H), 3.26 (s, 2H), 3.16-2.91 (m, 5H), 2.04-2.02 (m, 4H), 1.69 (s, 1H), 1.40-1.38 (m, 2H), 1.32-1.04 (m, 8H). Mass (m/z): 398.8 [M+H]⁺.

### Compound 468

### 5-((((1r,4r)-4-aminocyclohexyl)methyl)amino)-2-(2,6-dimethylmorpholino)-benzonitrile

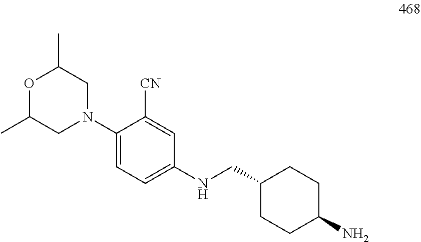 [SMILES: CC1CN(c2ccc(NC[C@H]3CC[C@H](N)CC3)cc2C#N)CC(C)O1]

The title compound 468 (84 mg) as a white solid was prepared from tert-butyl ((1r,4r)-4-(((3-cyano-4-(2,6-dimethylmorpholino)phenyl)amino)-methyl)cyclohexyl)carbamate (200 mg, 0.45 mmol), DCM (10 mL) and TFA (1 mL) according to the procedure for 24. ¹H NMR (400 MHZ, CD₃OD) δ 6.99 (d, J=8.8 Hz, 1H), 6.84 (dd, J=8.8, 2.8 Hz, 1H), 6.79 (d, J=2.8 Hz, 1H), 3.89-3.77 (m, 2H), 3.11 (d, J=10.8 Hz, 2H), 3.05 (td, J=8.0, 4.0 Hz, 1H), 2.94 (d, J=6.6 Hz, 2H), 2.46-2.37 (m, 2H), 2.02 (dd, J=31.0, 11.8 Hz, 4H), 1.65-1.53 (m, 1H), 1.40 (m, 2H), 1.18 (d, J=6.2 Hz, 6H), 1.10 (dd, J=19.2, 8.8 Hz, 2H). Mass (m/z): 343.3 [M+H]⁺.

### Compound 469

### N-(((1r,4r)-4-aminocyclohexyl)methyl)-4-(2,6-dimethylmorpholino)-2-fluoro-3-methylaniline

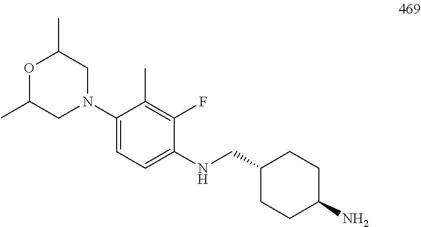 [SMILES: Cc1c(N2CC(C)OC(C)C2)ccc(NC[C@H]2CC[C@H](N)CC2)c1F]

The title compound 469 (141 mg) as a white solid was prepared from tert-butyl ((1r,4r)-4-(((4-(2,6-dimethylmorpholino)-2-fluoro-3-methylphenyl)-amino)methyl)cyclohexyl) carbamate (200 mg, 0.44 mmol), DCM (10 mL), and TFA (1 mL) according to the procedure for 24. ¹H NMR (400 MHZ, CD₃OD) δ 6.70 (dd, J=8.6, 1.2 Hz, 1H), 6.52 (t, J=9.2 Hz, 1H), 3.81 (brs, 2H), 3.05 (ddd, J=15.8, 7.8, 3.8 Hz, 1H), 3.00 (t, J=6.6 Hz, 2H), 2.81 (d, J=10.8 Hz, 2H), 2.39-2.26 (m, 2H), 2.17 (d, J=2.8 Hz, 3H), 2.01 (dd, J=31.0, 11.6 Hz, 4H), 1.67-1.53 (m, 1H), 1.37 (qd, J=12.6, 3.0 Hz, 2H), 1.16 (d, J=6.2 Hz, 6H), 1.14-1.03 (m, 2H). Mass (m/z): 350.3 [M+H]⁺.

### Compound 470

### N2-(4-(4-(2-ethoxyethoxy) piperidin-1-yl)-2-methylphenyl)spiro[3.3]heptane-2,6-diamine

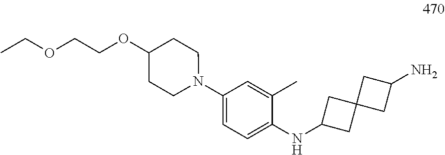 [SMILES: CCOCCOC1CCN(c2ccc(NC3CC4(CC(N)C4)C3)c(C)c2)CC1]

The title compound 470 (40 mg) as a yellow solid (40 mg, 33.5%) was prepared from tert-butyl (6-((4-(4-(2-ethoxyethoxy) piperidin-1-yl)-2-methylphenyl)amino)spiro[3.3]heptan-2-yl)carba mate (150 mg, 0.24 mmol), 1,4-dioxane (3 mL) and HCl/1,4-dioxane (1 mL) according to the procedure for 24. ¹H NMR (400 MHZ, DMSO-d₆) δ 6.66 (d, J=2.5 Hz, 1H), 6.59 (dd, J=8.5, 2.6 Hz, 1H), 6.27 (d, J=8.6 Hz, 1H), 4.36 (d, J=6.0 Hz, 1H), 3.66-3.59 (m, 1H), 3.55-3.51 (m, 2H), 3.47 (dd, J=4.4, 2.3 Hz, 2H), 3.44-3.41 (m, 2H), 3.20 (s, 3H), 2.65-2.57 (m, 2H), 2.45-2.38 (m, 1H), 2.30 (dt, J=11.5, 4.5 Hz, 2H), 2.15-2.09 (m, 1H), 2.02 (s, 3H), 1.93-1.81 (m, 4H), 1.68 (dd, J=14.3, 6.0 Hz, 2H), 1.55-1.47 (m, 2H), 1.10 (t, J=7.0 Hz, 3H). Mass (m/z): 388.3 [M+H]⁺.

### Compound 471

### N2-(4-(4-ethoxypiperidin-1-yl)-2-methylphenyl)spiro[3.3]heptane-2,6-diamine

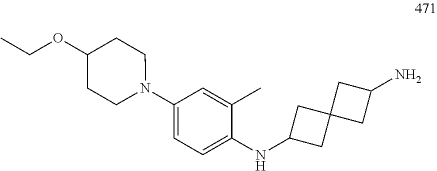 [SMILES: CCOC1CCN(c2ccc(NC3CC4(CC(N)C4)C3)c(C)c2)CC1]

The title compound 471 (47.7 mg, 19%) as a white solid was prepared from tert-butyl (6-((4-(4-ethoxypiperidin-1-yl)-2-methylphenyl)amino)spiro[3.3]heptan-2-yl)carbamate (319 mg, 0.72 mmol), HCl in dioxane (5 mL) and DCM (5 mL) according to the procedure for 37. 1¹H NMR (400 MHZ, CD₃OD) δ 6.83-6.73 (m, 2H), 6.47 (d, J=8.5 Hz, 1H), 3.81-3.73 (m, 1H), 3.56 (q, J=7.0 Hz, 2H), 3.49-3.40 (m, 1H), 3.29-3.22 (m, 2H), 2.78-2.69 (m, 2H), 2.57-2.50 (m, 1H), 2.48-2.41 (m, 1H), 2.40-2.32 (m, 1H), 2.28-2.21 (m, 1H), 2.11 (s, 3H), 2.06-1.96 (m, 2H), 1.94-1.76 (m, 4H), 1.74-1.62 (m, 2H), 1.19 (t, J=7.0 Hz, 3H). Mass (m/z): 344.3 [M+H]⁺.

### Compound 472

### N-(((1r,4r)-4-aminocyclohexyl)methyl)-4-(8-oxa-3-azabicyclo[3.2.1]octan-3-yl)-3-fluoroaniline

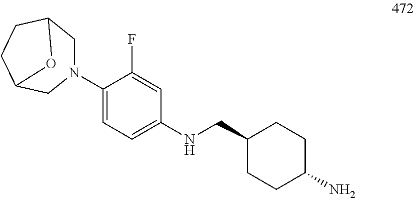 [SMILES: N[C@H]1CC[C@H](CNc2ccc(N3CC4CCC(C3)O4)c(F)c2)CC1]

The title compound 472 (19.5 mg) was prepared in a total yield of 58.4% as a gray solid according to the procedure for compound 24. ¹H NMR (400 MHZ, DMSO-d₆) δ 6.75 (m, 1H), 6.45-6.20 (m, 2H), 4.32-4.26 (m, 2H), 2.91 (m, 1H), 2.85-2.71 (m, 6H), 2.05-1.93 (m, 4H), 1.89-1.70 (m, 4H), 1.44-1.24 (m, 3H), 1.05-0.92 (m, 2H). Mass (m/z): 334.2 [M+H]⁺.

### Compound 473

### N1-(3-(2-methoxyethoxy)-4-(4-(trifluoromethyl)piperidin-1-yl)phenyl)cyclohexane-1,4-diamine

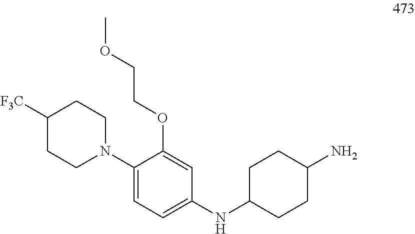 [SMILES: COCCOc1cc(NC2CCC(N)CC2)ccc1N1CCC(C(F)(F)F)CC1]

The title compound 473 (18.1 mg) was prepared in a total yield of 43.5% as a white solid according to the procedure for compound 24. ¹H NMR (400 MHZ, DMSO-d₆) δ 6.66 (s, 1H), 6.35-6.05 (m, 2H), 4.10-3.95 (m, 2H), 3.72-3.57 (m, 2H), 3.28 (s, 3H), 3.30-3.22 (m, 2H), 3.07 (m, 1H), 2.95 (m, 1H), 2.47-2.38 (m, 2H), 2.33 (m, 1H), 2.06-1.93 (m, 2H), 1.89-1.65 (m, 5H), 1.62-1.36 (m, 4H), 1.14 (m, 1H). Mass (m/z): 416.3 [M+H]⁺

### Compound 474

### N⁴-(6-(2.6-dimethylmorpholino)-2-methylpyridin-3-yl)adamantane-1,4-diamine

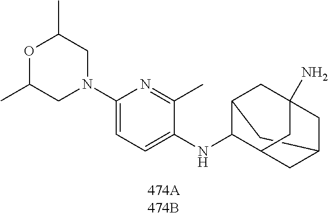 [SMILES: Cc1nc(N2CC(C)OC(C)C2)ccc1NC1C2CC3CC1CC(N)(C3)C2]

The title compound 474A and 474B were prepared according to the procedure for compound 24. The residue was purified by preparative HPLC (XSelect-CSH-Prep 5 μm OBD, 19*150 mm column; ACN/water (0.5% TFA)=0%-0%-30%-95%-95%-0%, 0 min-2 min-9 min-9.5 min-10.5 min-12.0 min) to afford compound 474A (Rt=7.35 min) in a 22.6% yield as a white solid and 474B (Rt=7.86 min) in a 14.0. % yield as a white solid. 474A: ¹H NMR (400 MHZ, DMSO-d₆) δ 6.84 (d, J=8.9 Hz, 1H), 6.48 (d, J=8.8 Hz, 1H), 3.83-3.78 (m, 3H), 3.61-3.54 (m, 2H), 3.41 (br, 3H), 3.31-3.28 (m, 1H), 2.24 (s, 3H), 2.12 (dd, J=12.4, 10.4 Hz, 2H), 1.97-1.85 (m, 5H), 1.69-1.62 (m, 2H), 1.61-1.53 (m, 4H), 1.31-1.25 (m, 2H), 1.10 (d, J=6.3 Hz, 6H). Mass (m/z): 374.2 [M+H]⁺. 474B: ¹H NMR (400 MHZ, DMSO-d₆) δ 7.51 (br, 3H), 6.90 (d, J=8.8 Hz, 1H), 6.52 (d, J=8.8 Hz, 1H), 3.91-3.81 (m, 3H), 3.65-3.57 (m, 2H), 3.30 (d, J=3.1 Hz, 1H), 2.31 (s, 3H), 2.21-2.12 (m, 4H), 2.11-2.04 (m, 3H), 1.80-1.63 (m, 6H), 1.54-1.46 (m, 2H), 1.14 (d, J=6.2 Hz, 6H). Mass (m/z): 374.2 [M+H]⁺.

### Compound 475

### N-(5-(aminomethyl) adamantan-2-yl)-6-(2,6-dimethylmorpholino)-2-methylpyridin-3-amine

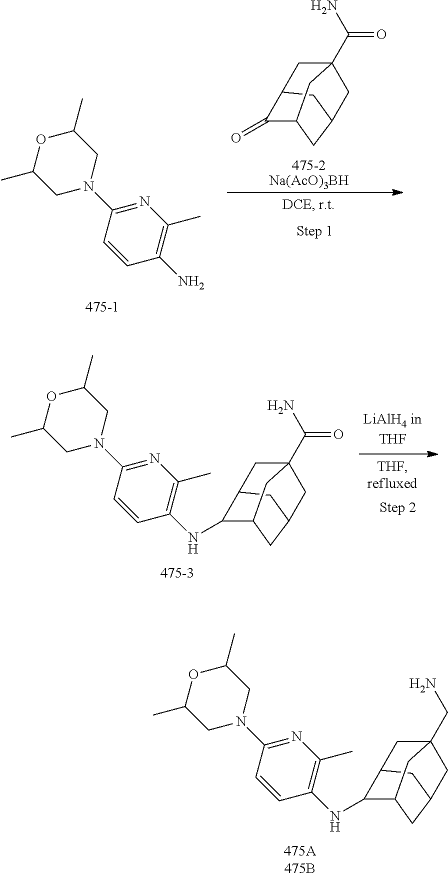

Step 1. Preparation of 4-((6-(2,6-dimethylmorpholino)-2-methylpyridin-3-yl)amino)adamantane-1-carboxamide (475-3)

The title compound 475-3 (16.0 mg) was prepared in a total yield of 68.7% as a yellow solid from 6-(2,6-dimethylmorpholino)-2-methylpyridin-3-amine (150 mg, 0.68 mmol), 4-oxoadamantane-1-carboxamide (131 mg, 0.68 mmol) and Na(AcO)₃BH (288 mg, 1.36 mmol) in DCE (5.0 mL) according to the procedure for 403-3. Mass (m/z): 399.2 [M+H]⁺.

Step 2. Preparation of N-(5-(aminomethyl) adamantan-2-yl)-6-(2,6-dimethylmorpholino)-2-methylpyridin-3-amine (475)

A solution of 4-((6-(2,6-dimethylmorpholino)-2-methylpyridin-3-yl)amino)adamantane-1-carboxamide (186 mg, 0.47 mol) in THF (10 mL) was added 0.39 mL of a solution of LiAlH₄ (35.7 mg, 0.6 mol) in THF at 0° C., and the mixture was refluxed for 2 h. After cooling to 0° C., water (35.7 uL), 10% NaOH (71.4 uL) and water 107.1 uL) were added, and the mixture was stirred for 3 min at room temperature. The solid was filtered and the filtered gray cake was washed with THF (10 mL*2); then the combined filtrates were dried over Na₂SO₄ and concentrated. The residue was purified by preparative HPLC (XSelect-CSH-Prep 5 μm OBD, 19*150 mm column; ACN/water (0.5% TFA)=0%-0%-40%-95%-95%-0%, 0 min-2 min-9 min-9.5 min-10.5 min-12 min) to afford compound 475A (Rt=7.89 min) in 14.5% yield as a white solid and 475B (Rt=8.56 min) in 6.3% yield as a white solid. 475A: ¹H NMR (400 MHZ, DMSO-d₆) δ 7.84 (s, 3H), 6.87 (d, J=8.6 Hz, 1H), 6.54 (d, J=8.8 Hz, 1H), 3.98-3.79 (m, 3H), 3.67-3.55 (m, 2H), 3.35-3.30 (m, 2H), 2.30 (s, 3H), 2.22-2.13 (m, 2H), 2.04-1.90 (m, 5H), 1.64-1.58 (m, 2H), 1.55-1.50 (m, 2H), 1.40-1.33 (m, 2H), 1.14 (d, J=6.2 Hz, 6H). Mass (m/z): 385.2 [M+H]⁺. 475B: ¹H NMR (400 MHZ, DMSO-d₆) δ 7.43 (s, 3H), 6.91 (d, J=8.8 Hz, 1H), 6.52 (d, J=8.7 Hz, 1H), 3.88-3.82 (m, 2H), 3.81-3.76 (m, 1H), 3.66-3.57 (m, 2H), 3.40-3.35 (m, 2H), 2.29 (s, 3H), 2.20-2.13 (m, 2H), 2.02-1.95 (m, 3H), 1.81-1.73 (m, 4H), 1.71-1.66 (m, 2H), 1.51-1.47 (m, 2H), 1.29-1.22 (m, 2H), 1.14 (d, J=6.2 Hz, 6H). Mass (m/z): 385.2 [M+H]⁺.

### Compound 476

### N2-(6-(2,6-dimethylmorpholino)-2,5-dimethylpyridin-3-yl)spiro[3.3]heptane-2, 6-diamine

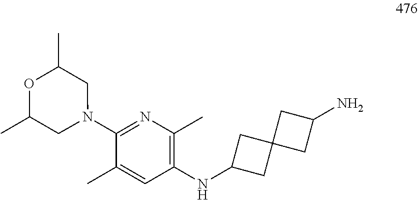 [SMILES: Cc1cc(NC2CC3(CC(N)C3)C2)c(C)nc1N1CC(C)OC(C)C1]

The title compound 476 (18.0 mg) was prepared in a total yield of 60.9% as a dark blue solid according to the procedure for compound 24. ¹H NMR (400 MHZ, Deuterium Oxide) δ 6.87 (s, 1H), 3.83-3.73 (m, 2H), 3.69-3.59 (m, 2H), 2.98 (d, J=12.2 Hz, 2H), 2.51-2.39 (m, 4H), 2.37-2.30 (m, 1H), 2.26-2.18 (m, 1H), 2.14-2.03 (m, 8H), 1.88-1.79 (m, 2H), 1.06 (d, J=6.3 Hz, 6H). Mass (m/z): 345.2 [M+H]⁺.

### Compound 477

### N2-(6-(2,6-dimethylmorpholino)pyridin-3-yl)spiro[3.3]heptane-2,6-diamine

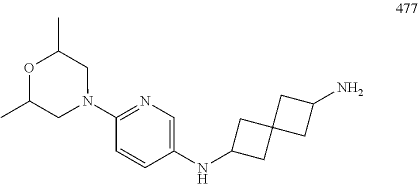 [SMILES: CC1CN(c2ccc(NC3CC4(CC(N)C4)C3)cn2)CC(C)O1]

The title compound 477 (55.2 mg) was prepared in a total yield of 53.1% as a green solid from tert-butyl (6-((6-(2,6-dimethylmorpholino)pyridin-3-yl)amino)spiro[3.3]heptan-2-yl)carbamate (125 mg, 0.300 mmol), TFA (1 mL), and DCM (10 mL) according to the procedure for 24. H NMR (400 MHZ, DMSO-d₆) δ 8.43 (s, 3H), 7.49 (d, J=2.8 Hz, 1H), 6.92 (dd, J=8.8, 2.8 Hz, 1H), 6.71 (d, J=9.2 Hz, 1H), 3.89-3.80 (m, 2H), 3.70-3.57 (m, 3H), 3.51 (d, J=8.4 Hz, 2H), 2.47 (td, J=6.8, 3.6 Hz, 1H), 2.35 (tdt, J=11.6, 6.8, 4.0 Hz, 2H), 2.27-2.15 (m, 5H), 1.82 (dt, J=11.2, 8.0 Hz, 2H), 1.13 (d, J=6.0 Hz, 6H). Mass (m/z): 317.3 [M+H]⁺.

### Compound 478

### N-(((1r,4S)-4-aminocyclohexyl)methyl)-4-((2S,6S)-2,6-dimethylmorpholino)-3-fluoroaniline

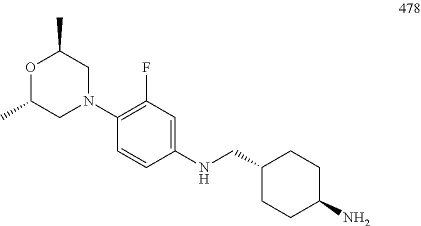 [SMILES: C[C@H]1CN(c2ccc(NC[C@H]3CC[C@H](N)CC3)cc2F)C[C@H](C)O1]

The title compound 478 (41.9 mg) was prepared in a total yield of 39.6% as a white solid from tert-butyl ((1S,4r)-4-(((4-((2S,6S)-2,6-dimethylmorpholino)-3-fluorophenyl)amino)methyl)cyclohexyl)carbamate (138 mg, 0.317 mmol), TFA (1 mL), and DCM (10 mL) according to the procedure for 24. ¹H NMR (400 MHz, DMSO-d₆) δ 8.31 (s, 3H), 6.77 (dd, J=10.0, 8.8 Hz, 1H), 6.39-6.26 (m, 2H), 5.68 (t, J=6.0 Hz, 1H), 3.99 (pd, J=6.4, 3.2 Hz, 2H), 2.93-2.75 (m, 5H), 2.58-2.52 (m, 2H), 2.05-1.94 (m, 2H), 1.90-1.78 (m, 2H), 1.51-1.27 (m, 3H), 1.20 (d, J=6.4 Hz, 6H), 0.98 (tdd, J=15.2, 11.2, 4.8 Hz, 2H). Mass (m/z): 336.3 [M+H]⁺.

### Compound 479

### N2-(6-((2S,6S)-2,6-dimethylmorpholino)-2-methylpyridin-3-yl)spiro[3.3]heptane-2,6-diamine

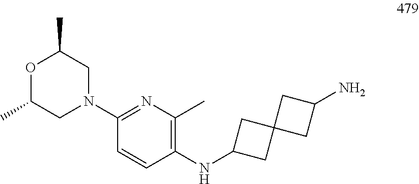 [SMILES: Cc1nc(N2C[C@H](C)O[C@@H](C)C2)ccc1NC1CC2(CC(N)C2)C1]

The title compound 479 (51.5 mg) was prepared in a total yield of 61.6% as a yellow solid from tert-butyl (6-((6-((2S,6S)-2,6-dimethylmorpholino)-2-methylpyridin-3-yl)amino)spiro[3.3]heptan-2-yl)carbamate (109 mg, 0.253 mmol), TFA (1 mL), and DCM (10 mL) according to the procedure for 24. ¹H NMR (400 MHZ, DMSO-d₆) δ 8.41 (s, 3H), 6.76 (s, 1H), 6.50 (d, J=8.4 Hz, 1H), 4.60 (s, 1H), 3.99 (tt, J=10.0, 4.8 Hz, 2H), 3.71-3.59 (m, 1H), 3.53 (s, 1H), 3.30 (dd, J=12.4, 3.2 Hz, 2H), 2.92 (dd, J=12.0, 6.0 Hz, 2H), 2.47 (t, J=5.6 Hz, 1H), 2.36 (dddd, J=20.0, 11.6, 7.2, 4.0 Hz, 2H), 2.26-2.15 (m, 6H), 1.92 (p, J=6.4 Hz, 2H), 1.17 (d, J=6.4 Hz, 6H). Mass (m/z): 331.3 [M+H]⁺.

### Compound 480

### N2-(6-((25,6S)-2,6-dimethylmorpholino)-5-methylpyridin-3-yl)spiro[3.3]heptane-2,6-diamine

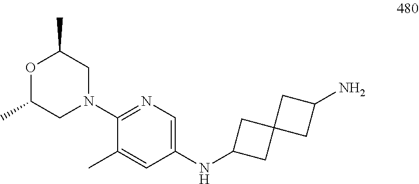 [SMILES: Cc1cc(NC2CC3(CC(N)C3)C2)cnc1N1C[C@H](C)O[C@@H](C)C1]

The title compound 480 (47.4 mg) was prepared in a total yield of 55.6% as a yellow solid from tert-butyl (6-((6-((2S,6S)-2,6-dimethylmorpholino)-5-methylpyridin-3-yl)amino)spiro[3.3]heptan-2-yl)carbamate (111 mg, 0.258 mmol), TFA (1 mL), and DCM (10 mL) according to the procedure for 24. ¹H NMR (400 MHZ, DMSO-d₆) δ 8.35 (s, 3H), 7.42 (d, J=2.8 Hz, 1H), 6.75 (d, J=2.8 Hz, 1H), 5.71 (s, 1H), 3.73-3.64 (m, 3H), 3.00-2.89 (m, 2H), 2.51-2.46 (m, 1H), 2.37 (qd, J=11.6, 6.4 Hz, 4H), 2.24-2.16 (m, 3H), 2.15 (s, 3H), 1.82 (dt, J=11.2, 8.4 Hz, 2H), 1.08 (d, J=6.0 Hz, 6H). Mass (m/z): 331.3 [M+H]⁺.

### Compound 481

### N-(((1r,4r)-4-aminocyclohexyl)methyl)-4-(2,6-dimethylmorpholino)-2,5-difluoroaniline

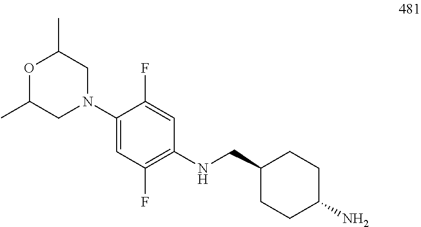 [SMILES: CC1CN(c2cc(F)c(NC[C@H]3CC[C@H](N)CC3)cc2F)CC(C)O1]

The title compound 481 (4 mg) as a white solid was prepared according to the procedure for 24. 1¹H NMR (400 MHz, DMSO-d₆) δ 6.79 (dd, J=13.3, 8.0 Hz, 1H), 6.51 (dd, J=14.2, 8.0 Hz, 1H), 5.35-5.24 (m, 1H), 3.69 (ddt, J=12.7, 6.5, 3.2 Hz, 2H), 3.05-2.95 (m, 2H), 2.84 (t, J=6.4 Hz, 2H), 2.25 (t, J=10.8 Hz, 2H), 1.99 (p, J=7.0, 6.5 Hz, 1H), 1.76 (dd, J=22.6, 10.2 Hz, 4H), 1.45 (d, J=12.5 Hz, 1H), 1.31-1.18 (m, 4H), 1.09 (d, J=6.2 Hz, 6H). Mass (m/z): 354.2 [M+H]⁺.

### Compound 482

### N2-(2-methyl-4-(2-oxa-6-azaspiro[3.3]heptan-6-yl)phenyl)spiro[3.3]heptane-2,6-diamine

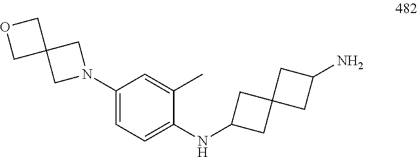 [SMILES: Cc1cc(N2CC3(COC3)C2)ccc1NC1CC2(CC(N)C2)C1]

The title compound 482 (24.1 mg, 16.8%) as oil was prepared from tert-butyl (6-((2-methyl-4-(2-oxa-6-azaspiro[3.3]heptan-6-yl)phenyl)amino)spiro[3.3]heptan-2-yl)carbamate (160 mg, 0.39 mmol), DCM (10 mL) and TFA (1 mL) according to the procedure for 24. 1¹H NMR (400 MHZ, CD₃OD) § 6.46 (m, 1H), 6.33 (m, 2H), 4.79 (s, 4H), 3.88 (brs, 4H), 3.74 (brs, 1H), 3.42 (t, J=7.6 Hz, 1H), 2.57-2.50 (m, 1H), 2.51-2.44 (m, 1H), 2.38 (m, 1H), 2.30-2.22 (m, 1H), 2.10 (s, 3H), 1.92 (m, 4H). Mass (m/z): 314.22 [M+H]⁺.

### Compound 483

### N-(((1r,4r)-4-aminocyclohexyl)methyl)-2-chloro-4-(2,6-dimethylmorpholino) aniline

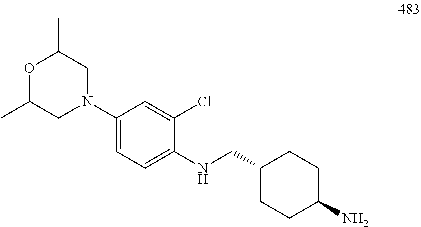 [SMILES: CC1CN(c2ccc(NC[C@H]3CC[C@H](N)CC3)c(Cl)c2)CC(C)O1]

The title compound 483 (29.8 mg, yield: 75.9%) as black oil was prepared from tert-butyl ((1r,4r)-4-(((2-chloro-4-(2,6-dimethylmorpholino)phenyl)amino)methyl)cyclohexyl)carbamate (90 mg, 0.2 mmol), DCM (20 mL) and TFA (4 mL) according to the procedure for 24. ¹H NMR (400 MHZ, CD₃OD) δ 7.18 (dd, J=9.2, 2.9 Hz, 1H), 6.96 (d, J=2.8 Hz, 1H), 6.73 (d, J=9.2 Hz, 1H), 3.77 (ddd, J=10.2, 6.3, 2.2 Hz, 2H), 3.30-3.24 (m, 2H), 3.04 (d, J=6.8 Hz, 2H), 2.64 (s, 1H), 2.28-2.19 (m, 2H), 1.96-1.83 (m, 4H), 1.64-1.50 (m, 1H), 1.24-0.99 (m, 10H). Mass (m/z): 351.8 [M+H]⁺.

### Compound 484

### 2-((((1r,4r)-4-aminocyclohexyl)methyl)amino)-5-(2,6-dimethylmorpholino) benzonitrile

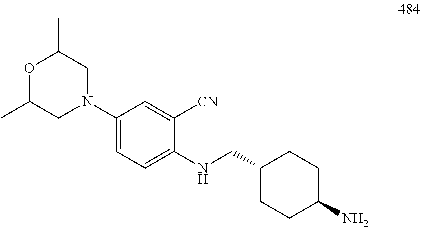 [SMILES: CC1CN(c2ccc(NC[C@H]3CC[C@H](N)CC3)c(C#N)c2)CC(C)O1]

The title compound 484 (29 mg, yield: 37.2%) as a blue solid was prepared from tert-butyl ((1r,4r)-4-(((2-cyano-4-(2,6-dimethylmorpholino)phenyl)amino)methyl)cyclohexyl)carbamate (100 mg, 0.21 mmol), DCM (20 mL) and TFA (4 mL) according to the procedure for 24. H NMR (400 MHZ, CD₃OD) δ 6.92 (d, J=2.7 Hz, 1H), 6.84 (dd, J=8.9, 2.7 Hz, 1H), 6.67 (d, J=8.9 Hz, 1H), 3.77 (ddd, J=10.2, 6.3, 2.2 Hz, 2H), 3.27 (d, J=10.6 Hz, 2H), 3.00 (d, J=6.8 Hz, 2H), 2.67 (tt, J=11.2, 3.8 Hz, 1H), 2.29-2.19 (m, 2H), 1.99-1.83 (m, 4H), 1.56 (ddd, J=11.1, 7.7, 4.0 Hz, 1H), 1.16 (dd, J=32.3, 17.3 Hz, 10H). Mass (m/z): 342.9 [M+H]⁺.

### Compound 485

### 2-(2-((4-aminocyclohexyl)amino)-5-(4-(trifluoromethyl)piperidin-1-yl) phenoxy) ethan-1-ol

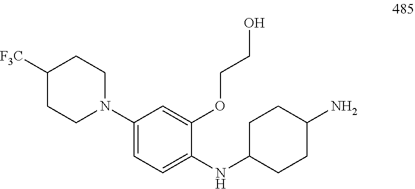 [SMILES: NC1CCC(Nc2ccc(N3CCC(C(F)(F)F)CC3)cc2OCCO)CC1]

The desired product as a yellow solid (4.7 mg, 5.8%) was prepared from tert-butyl (4-((2-(2-hydroxyethoxy)-4-(4-(trifluoromethyl)piperidin-1-yl)phenyl)amino)cyclohexyl)carbamate (100 mg, 0.20 mmol), 1,4-dioxane (3 mL) and HCl/1,4-Dioxane (1 mL) according to the procedure for 37. ¹H NMR (400 MHZ, CD₃OD) δ 8.54 (s, 2H), 6.64 (d, J=8.3 Hz, 2H), 6.57 (s, 1H), 4.06 (s, 2H), 3.92-3.88 (m, 2H), 3.50 (dd, J=13.8, 6.7 Hz, 3H), 3.34 (s, 1H), 2.97 (s, 1H), 2.65 (s, 3H), 2.24 (s, 1H), 1.96 (d, J=11.9 Hz, 2H), 1.72 (m, 10H). Mass (m/z): 402.2 [M+H]⁺.

### Compound 486

### N2-(6-(8-oxa-3-azabicyclo[3.2.1]octan-3-yl)-2-methylpyridin-3-yl)spiro[3.3]heptane-2,6-diamine

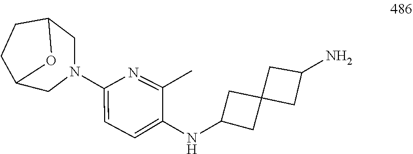 [SMILES: Cc1nc(N2CC3CCC(C2)O3)ccc1NC1CC2(CC(N)C2)C1]

The title compound 486 (50.2 mg) was prepared in a total yield of 76.3% as a purple solid according to the procedure for compound 24. ¹H NMR (400 MHZ, DMSO-d₆) δ 6.72 (d, J=8.8 Hz, 1H), 6.37 (d, J=8.8 Hz, 1H), 4.41-4.30 (m, 2H), 3.69-3.46 (m, 3H), 3.16 (m, 1H), 2.78-2.67 (m, 2H), 2.43-2.30 (m, 2H), 2.25-2.06 (m, 7H), 1.96-1.84 (m, 2H), 1.80-1.68 (m, 4H). Mass (m/z): 329.2 [M+H]⁺.

### Compound 487

### N2-(6-((2S,6R)-2,6-dimethylmorpholino)-4-methylpyridin-3-yl)spiro[3.3]heptane-2,6-diamine

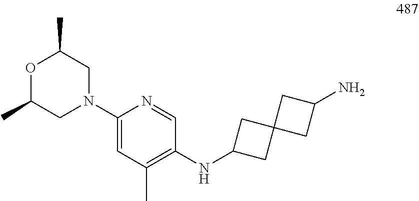 [SMILES: Cc1cc(N2C[C@@H](C)O[C@@H](C)C2)ncc1NC1CC2(CC(N)C2)C1]

The title compound 487 (52.1 mg) was prepared in a total yield of 52.4% as a white solid according to the procedure for compound 24. ¹H NMR (400 MHZ, DMSO-d₆) δ 7.26 (s, 1H), 6.69 (s, 1H), 3.95-3.82 (m, 2H), 3.76-3.47 (m, 5H), 3.16 (m, 1H), 2.44-2.30 (m, 2H), 2.27-2.12 (m, 4H), 2.09 (s, 3H), 1.97-1.86 (m, 2H), 1.12 (d, J=6.4 Hz, 6H). Mass (m/z): 331.2 [M+H]⁺.

### Compound 488

### N2-(6-(2,6-dimethylmorpholino)-2-methylpyridin-3-yl)spiro[3.3]heptane-2,6-diamine

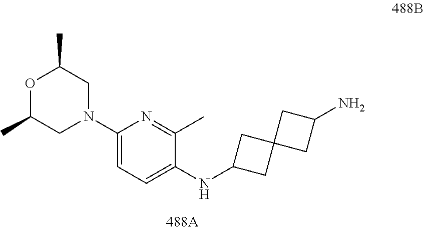 [SMILES: Cc1nc(N2C[C@@H](C)O[C@@H](C)C2)ccc1NC1CC2(CC(N)C2)C1]

The title compound 488 was prepared according to the procedure for compound 24. The mixture was purified by CHIRALPAK IG-3, 4.6*50 mm, 3 μm (solvent system (MTBE (3% 0 isopropylamine): EtOH=80:20) to afford compound 488A (Rt=1.614 min) in 13.6% yield as a light yellow solid and 488B (Rt=2.177 min) in 14.5% yield as a light yellow solid. 488A: ¹H NMR (400 MHZ, Methanol-d₄) δ 6.86 (d, J=8.4 Hz, 1H), 6.52 (d, J=8.4 Hz, 1H), 3.85-3.64 (m, 5H), 3.27 (m, 1H), 2.56-2.39 (m, 2H), 2.35 (m, 1H), 2.31-2.22 (m, 6H), 1.96-1.75 (m, 4H), 1.21 (d, J=6.0 Hz, 6H). Mass (m/z): 331.2 [M+H]⁺. 488B: ¹H NMR (400 MHZ, Methanol-d₄) δ 6.87 (d, J=8.8 Hz, 1H), 6.53 (d, J=8.8 Hz, 1H), 3.85-3.64 (m, 5H), 3.27 (m, 1H), 2.62-2.40 (m, 2H), 2.38-2.32 (m, 2H), 2.28 (s, 3H), 2.26-2.19 (m, 2H), 1.97-1.72 (m, 4H), 1.21 (d, J=6.0 Hz, 6H). Mass (m/z): 331.2 [M+H]⁺.

### Compound 489

### 3-(3-methyl-4-(4-methylpiperidin-1-yl)phenyl)amino)cyclobutane-1-carbohydrazide

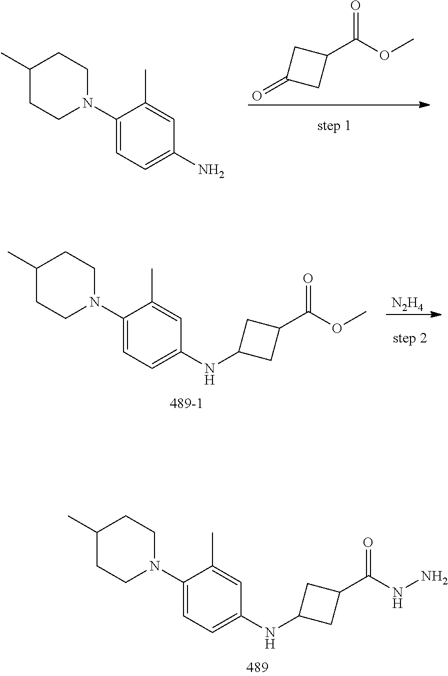

Step 1. Preparation of methyl 3-((3-methyl-4-(4-methylpiperidin-1-yl)phenyl)amino)cyclobutane-1-carboxylate (489-1)

To a solution of 3-methyl-4-(4-methylpiperidin-1-yl)aniline (0.2 g, 0.98 mmol) in DCE (5 ml) was added methyl 3-oxocyclobutane-1-carboxylate (125 mg, 0.98 mmol) and NaBH (OAc) 3 (623 mg, 2.94 mmol). The reaction was stirred for 3 h at R.T. Quenched with water (10 mL), the reaction was extracted by EA (10 mL) for 3 times. The combined organic layers were dried over sodium sulfate, concentrated and purified by flash, eluted with PE/EA=10:1 to 1:1 to give the desired product as yellow oil (0.22 g, 60%). Mass (m/z): 316.9 [M+H]⁺.

Step 2 Preparation of 3-((3-methyl-4-(4-methylpiperidin-1-yl)phenyl)amino)cyclobutane-1-carbohydrazide (489)

To a solution of methyl 3-((3-methyl-4-(4-methylpiperidin-1-yl)phenyl)amino)cyclobutane-1-carboxylate (220 mg, 0.7 mmol) in EtOH (10 mL) was added N₂H₄ (50 mg) and the mixture was stirred at 80° C. for 2 h. Quenched with water (30 mL), the reaction was extracted by EA (10 mL) for 3 times. The combined organic layers were dried over sodium sulfate, removed under vacuum and the residue was purified by preparative HPLC (column-Gemini-C18 150×21.2 mm, 5 μm; Mobile phase: ACN-H₂O (0.1% FA), 10%-40%) to afford compound 489 (83 mg, 42.9%) as a yellow solid. Mass (m/z): 316.8 [M+H]⁺. ¹H NMR (400 MHZ, DMSO-d₆) δ 6.79 (dd, J=8.4, 3.6, 1H), 6.35-6.22 (m, 2H), 5.55-5.50 (m, 1H), 3.91-3.68 (m, 1H), 3.64 (s, 1H), 3.59 (s, 2H), 2.84-2.81 (m, 2H), 2.55-2.52 (m, 1H), 2.47-2.44 (m, 1H), 2.12-2.04 (m, 4H), 1.98-1.91 (m, 1H), 1.65-1.63 (m, 2H), 1.42-1.37 (m, 1H), 1.29-1.20 (m, 2H), 0.94 (d, J=6.4, 3H). Mass (m/z): 317.3 [M+H]⁺.

### Compound 490

### N-(2-(3-aminocyclopentyl)ethyl)-6-(2,6-dimethylmorpholino)-2-methylpyridin-3-amine

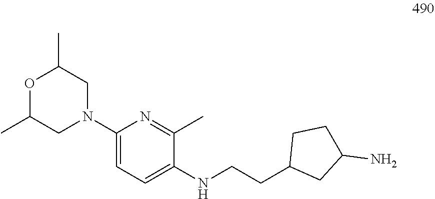 [SMILES: Cc1nc(N2CC(C)OC(C)C2)ccc1NCCC1CCC(N)C1]

The title compound 490 (40.4 mg, 9.4% yield) as a yellow solid was prepared from N-(4-aminophenyl)-2,2,2-trifluoro-N-methylacetamide (0.45 g, 1.3 mmol), BH₃-THF (10 mL) and THF (5 mL) according to the procedure for 150. ¹H NMR (400 MHZ, DMSO-d₆) δ=6.83 (d, J=8.8, 1H), 6.51 (d, J=8.8, 1H), 4.30 (s, 1H), 3.83 (d, J=11.2, 2H), 3.63-3.60 (m, 2H), 3.29-3.14 (m, 1H), 2.94 (t, J=7.2, 2H), 2.21 (s, 3H), 2.09-1.94 (m, 1H), 1.89-1.79 (m, 2H), 1.78-1.65 (m, 1H), 1.65-1.40 (m, 3H), 1.39-1.17 (m, 2H), 1.14 (d, J=6.4, 7H). Mass (m/z): 332.9 [M+H]⁺.

### Compound 491

### N2-(6-(2-oxa-5-azabicyclo[2.2.2]octan-5-yl)-2-methylpyridin-3-yl)spiro[3.3]-heptane-2,6-diamine

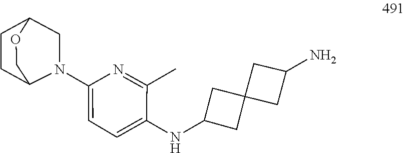 [SMILES: Cc1nc(N2CC3CCC2CO3)ccc1NC1CC2(CC(N)C2)C1]

The title compound (35 mg) as a white solid was prepared from tert-butyl (6-((6-(2-oxa-5-azabicyclo[2.2.2]octan-5-yl)-2-methylpyridin-3-yl)amino)spiro[3.3]heptan-2-yl)carbamate (400 mg, 0.84 mmol), DCM (10 mL) and TFA (1 mL) according to the procedure for 24. ¹HNMR (400 MHZ, DMSO-d₆) δ 6.71 (d, J=8.8 Hz, 1H), 6.18 (d, J=8.8 Hz, 1H), 4.37 (s, 1H), 3.90-3.88 (m, 1H), 3.86 (t, J=5.6 Hz, 2H), 3.79-3.75 (m, 0.4H), 3.53 (br, 2H), 3.31 (d, J=10.4 Hz, 1H), 3.15 (m, 1H), 2.44-2.32 (m, 1H), 2.31-2.25 (m, 1H), 2.24-2.17 (m, 1H), 2.16-2.12 (m, 3H), 2.12-2.05 (m, 1H), 2.00-1.91 (m, 1H), 1.89-1.70 (m, 5H), 1.68-1.57 (m, 2H). Mass (m/z): 329.3 [M+H]⁺.

### Compound 492

### 2-((3-methyl-4-(4-methylpiperidin-1-yl)phenyl)amino)cyclohexane-1-carboxamide

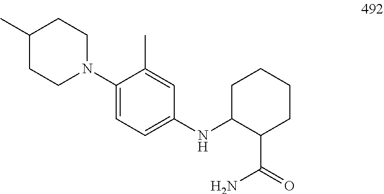 [SMILES: Cc1cc(NC2CCCCC2C(N)=O)ccc1N1CCC(C)CC1]

The title compound (141 mg) as a white solid was prepared from 2-((3-methyl-4-(4-methylpiperidin-1-yl)phenyl)amino)cyclohexane-1-carbox-ylic acid (100 mg, 0.3 mmol), DMF (15 mL), NH₄Cl (19.42 mg, 0.36 mmol), HATU (172.59 mg, 0.45 mmol) and DIEA (117.32 mg, 0.9 mmol) according to the procedure for 1. ¹H NMR (400 MHZ, DMSO-d₆) δ 7.18 (s, 1H), 6.74 (d, J=8.6 Hz, 1H), 6.71 (s, 1H), 6.41 (d, J=2.6 Hz, 1H), 6.35 (dd, J=8.4, 2.7 Hz, 1H), 3.63 (d, J=3.2 Hz, 1H), 2.79 (d, J=11.6 Hz, 2H), 2.46-2.39 (m, 3H), 2.08 (s, 3H), 1.79 (br, 2H), 1.64-1.21 (m, 12H), 0.90 (d, J=6.6 Hz, 3H). Mass (m/z): 330.3 [M+H]⁺.

### Compound 493

### N2-(6-(3,5-dimethyl-4-(2,2,2-trifluoroethyl) piperazin-1-yl)-2-methylpyridin-3-yl)spiro[3.3]heptane-2,6-diamine

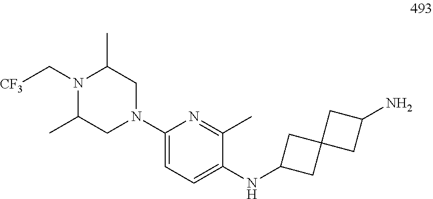 [SMILES: Cc1nc(N2CC(C)N(CC(F)(F)F)C(C)C2)ccc1NC1CC2(CC(N)C2)C1]

The title compound 493 (40 mg, 25%) as a yellow oil was prepared from tert-butyl (6-((6-(3,5-dimethyl-4-(2,2,2-trifluoroethyl) piperazin-1-yl)-2-methylpyridin-3-yl)amino)spiro[3.3]heptan-2-yl)carbamate (0.2 g, 0.4 mmol), DCM (5 mL) and TFA (1 mL) according to the procedure for 24. ¹H NMR (400 MHZ, CD₃OD) δ 6.84 (d, J=8.6 Hz, 1H), 6.52 (d, J=8.6 Hz, 1H), 3.74-3.68 (m, 3H), 3.41-3.30 (m, 3H), 2.87-2.83 (m, 2H), 2.57-2.32 (m, 5H), 2.28-2.22 (m, 4H), 1.94-1.83 (m, 4H), 1.14 (d, J=6.2 Hz, 6H). Mass (m/z): 411.8 [M+H]⁺.

### Compound 494

N2-(2-methyl-6-(7-oxa-2-azaspiro[3.5]nonan-2-yl)pyridin-3-yl)spiro[3.3]heptane-2,6-diamine

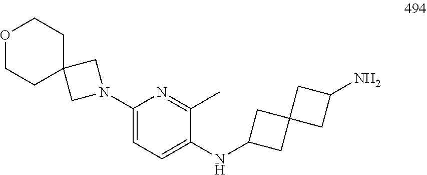 [SMILES: Cc1nc(N2CC3(CCOCC3)C2)ccc1NC1CC2(CC(N)C2)C1]

The title compound 494 (4.1 mg, 3.1%) as a yellow solid was prepared from (4-((2-(2-hydroxyethoxy)-4-(4-(trifluoromethyl)piperidin-1-yl)phenyl)amino)cyclohexyl)carbamate (170 mg, 0.38 mmol), 1,4-dioxane (3 mL) and HCl/1,4-dioxane (1 mL) according to the procedure for 37. ¹H NMR (400 MHZ, CD₃OD) δ 7.15 (d, J=8.4 Hz, 1H), 6.41 (d, J=8.3 Hz, 1H), 3.69 (m, 5H), 2.64-2.00 (m, 7H), 1.86-1.79 (m, 4H), 1.70-1.60 (m, 4H). Mass (m/z): 342.8 [M+H]⁺.

### Compound 495

### N-(((1r,4R)-4-aminocyclohexyl)methyl)-4-((2S,6R)-2,6-dimethylmorpholino)-3-methoxyaniline

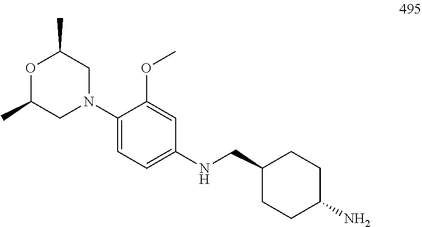 [SMILES: COc1cc(NC[C@H]2CC[C@H](N)CC2)ccc1N1C[C@@H](C)O[C@@H](C)C1]

The title compound 495 (57.2 mg) was prepared in a yield of 55.6% as a white powder from 4-(2,6-dimethylmorpholino)-3-methoxyaniline (70 mg, 0.30 mmol) and tert-butyl ((1r,4r)-4-formylcyclohexyl)carbamate (101 mg, 0.44 mmol) according to the procedure for 20. 1¹H NMR (400 MHZ, DMSO-d₆) δ 8.13 (s, 2H), 6.65 (s, 1H), 6.25 (s, 1H), 6.05 (s, 1H), 5.34 (s, 1H), 3.71 (s, 3H), 2.96 (d, J=39.4 Hz, 3H), 2.81 (s, 2H), 2.17 (s, 2H), 2.02-1.93 (m, 2H), 1.86 (d, J=13.0 Hz, 2H), 1.46 (s, 1H), 1.31 (td, J=13.4, 10.1 Hz, 3H), 1.08 (d, J=6.3 Hz, 6H), 1.01 (d, J=12.8 Hz, 2H). Mass (m/z): 348.3 [M+H]⁺.

### Compound 496

### N-(((1r,4R)-4-aminocyclohexyl)methyl)-4-((2S,6R)-2,6-dimethylmorpholino)-3-methoxyaniline

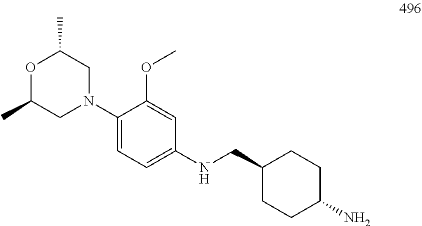 [SMILES: COc1cc(NC[C@H]2CC[C@H](N)CC2)ccc1N1C[C@@H](C)O[C@H](C)C1]

The title compound 496 (13.7 mg) was prepared in a yield of 14.1% as a white powder from 4-(2,6-dimethylmorpholino)-3-methoxyaniline (70 mg, 0.30 mmol) and tert-butyl ((1r,4r)-4-formylcyclohexyl)carbamate (101 mg, 0.44 mmol) according to the procedure for 20. ¹H NMR (400 MHZ, DMSO-d₆) δ 8.05 (s, 2H), 6.64 (s, 1H), 6.24 (s, 1H), 6.04 (s, 1H), 3.70 (s, 5H), 2.95 (d, J=39.1 Hz, 3H), 2.81 (s, 2H), 2.16 (s, 1H), 1.95 (d, J=12.0 Hz, 2H), 1.85 (d, J=12.9 Hz, 2H), 1.45 (s, 1H), 1.35-1.21 (m, 3H), 1.07 (d, J=6.3 Hz, 6H), 1.03-0.93 (m, 2H). Mass (m/z): 348.3 [M+H]⁺.

### Compound 497

### N-(((1r,4r)-4-aminocyclohexyl)methyl)-4-(2,6-dimethylmorpholino)-2-methoxyaniline

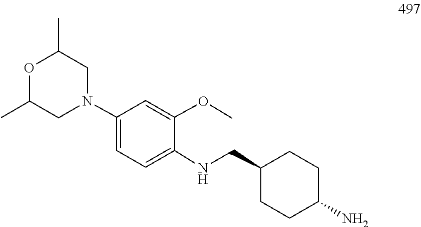 [SMILES: COc1cc(N2CC(C)OC(C)C2)ccc1NC[C@H]1CC[C@H](N)CC1]

The title compound 497 (46.7 mg) was prepared in a yield of 45.4% as a white powder from 4-(2,6-dimethylmorpholino)-2-methoxyaniline (70 mg, 0.30 mmol) and tert-butyl ((1r,4r)-4-formylcyclohexyl)carbamate (101 mg, 0.44 mmol) according to the procedure for 20. 1¹H NMR (400 MHZ, DMSO-d₆) δ 8.04 (s, 3H), 6.55 (s, 1H), 6.37 (d, J=22.1 Hz, 2H), 4.36 (s, 1H), 3.78 (d, J=6.0 Hz, 3H), 3.68 (s, 2H), 2.88 (d, J=24.0 Hz, 3H), 2.14 (s, 2H), 1.95 (s, 2H), 1.82 (d, J=12.7 Hz, 2H), 1.50 (s, 1H), 1.28 (q, J=13.3, 12.7 Hz, 3H), 1.19-1.08 (m, 6H), 0.99 (s, 2H). Mass (m/z): 348.7 [M+H]⁺.

### Compound 498

### N2-(6-(4-methoxy-4-(trifluoromethyl)piperidin-1-yl)-2-methylpyridin-3-yl)spiro[3.3]heptane-2, 6-diamine

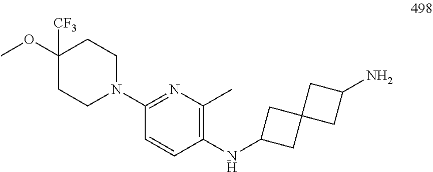 [SMILES: COC1(C(F)(F)F)CCN(c2ccc(NC3CC4(CC(N)C4)C3)c(C)n2)CC1]

The title compound 498 (6.0 mg) was prepared in a total yield of 11.0% as a white solid from tert-butyl (6-((6-(4-methoxy-4-(trifluoromethyl)piperidin-1-yl)-2-methylpyridin-3-yl)amino)spiro[3.3]heptan-2-yl)carbamate (68 mg, 0.137 mmol), TFA (1 mL), and DCM (10 mL) according to the procedure for 24. ¹H NMR (400 MHZ, DMSO-d₆) δ 6.72 (dd, J=8.8, 4.0 Hz, 1H), 6.55 (d, J=8.8 Hz, 1H), 4.52 (d, J=7.2 Hz, 1H), 3.94 (d, J=12.8 Hz, 2H), 3.84 (q, J=8.0 Hz, 1H), 3.61 (q, J=7.4 Hz, 1H), 3.38 (s, 3H), 2.74 (td, J=12.8, 2.4 Hz, 2H), 2.46-2.40 (m, 1H), 2.27 (td, J=10.4, 6.0 Hz, 2H), 2.20 (s, 3H), 2.10 (dd, J=11.2, 6.0 Hz, 1H), 1.97-1.64 (m, 8H). Mass (m/z): 399.3 [M+H]⁺.

### Compound 499

### N2-(2-methyl-6-(1,4-oxazepan-4-yl)pyridin-3-yl)spiro[3.3]heptane-2,6-diamine

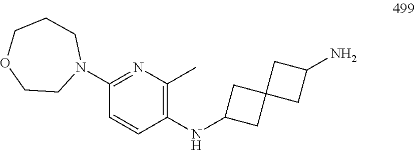 [SMILES: Cc1nc(N2CCCOCC2)ccc1NC1CC2(CC(N)C2)C1]

The title compound 499 (41.1 mg) was prepared in a total yield of 31.2% as a yellow solid from tert-butyl (6-((2-methyl-6-(1,4-oxazepan-4-yl)pyridin-3-yl)amino)spiro[3.3]heptan-2-yl)carbamate (158 mg, 0.380 mmol), TFA (1 mL), and DCM (10 mL) according to the procedure for 24. ¹H NMR (400 MHZ, DMSO-d₆) δ 8.44 (s, 3H), 6.86 (s, 1H), 6.40 (d, J=8.4 Hz, 1H), 3.70-3.58 (m, 8H), 3.55 (t, J=5.6 Hz, 3H), 2.49-2.42 (m, 1H), 2.35 (dddd, J=19.6, 11.2, 7.2, 2.8 Hz, 3H), 2.25 (d, J=8.0 Hz, 4H), 2.21-2.14 (m, 3H), 1.96 (s, 2H), 1.84 (p, J=5.6 Hz, 2H). Mass (m/z): 317.3 [M+H]⁺.

### Compound 500

### N-(((1r,4R)-4-aminocyclohexyl)methyl)-6-((2R,6S)-2,6-diethylmorpholino)-2-methylpyridin-3-amine

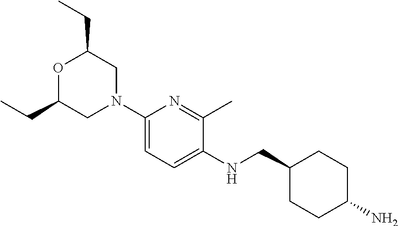 [SMILES: CC[C@@H]1CN(c2ccc(NC[C@H]3CC[C@H](N)CC3)c(C)n2)C[C@H](CC)O1]

The title compound 500 (30.8 mg) was prepared in a total yield of 53.1% as a light yellow solid according to the procedure for compound 24. ¹H NMR (400 MHZ, DMSO-d₆) δ 7.75 (s, 2H), 6.83 (d, J=8.7 Hz, 1H), 6.52 (d, J=8.7 Hz, 1H), 4.44 (s, 1H), 3.84 (d, J=11.9 Hz, 2H), 3.41-3.36 (m, 2H), 2.98-2.91 (m, 1H), 2.88-2.79 (m, 2H), 2.22 (s, 3H), 2.21-2.14 (m, 2H), 1.97-1.84 (m, 4H), 1.51-1.45 (m, 4H), 1.30-1.25 (m, 2H), 1.06-0.90 (m, 8H). Mass (m/z): 361.3 [M+H]⁺.

### Compound 501

### N²-(6-((2R,6S)-2,6-dimethylmorpholino)-4-methoxypyridin-3-yl)spiro[3.3]heptane-2,6-diamine

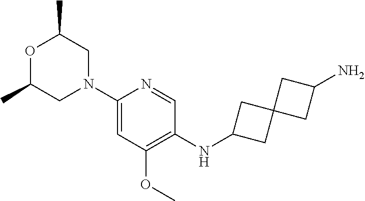 [SMILES: COc1cc(N2C[C@@H](C)O[C@@H](C)C2)ncc1NC1CC2(CC(N)C2)C1]

The title compound 501 (28.8 mg) was prepared in a total yield of 16.6% as a light yellow solid according to the procedure for compound 34. ¹H NMR (400 MHZ, DMSO-d₆) δ 8.26 (s, 3H), 7.06 (s, 1H), 6.51 (s, 1H), 4.04-3.97 (m, 2H), 3.90 (s, 3H), 3.69-3.58 (m, 3H), 3.56-3.50 (m, 1H), 3.41-3.29 (m, 2H), 2.50-2.45 (m, 2H), 2.42-2.30 (m, 4H), 2.23-2.13 (m, 3H), 1.96-1.86 (m, 2H). Mass (m/z): 347.2 [M+H]⁺.

### Compound 502

### (1R,3S)-3-amino-4-((4-(4-(trifluoromethyl)piperidin-1-yl)phenyl)amino)cyclohexane-1-carboxamide

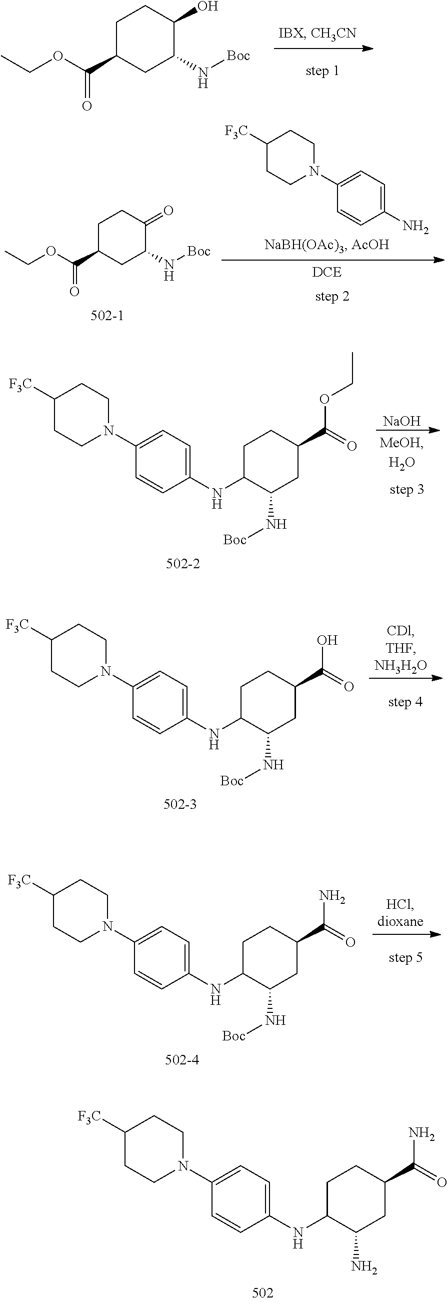

Step 1. Preparation of ethyl (1S,3R)-3-((tert-butoxycarbonyl)amino)-4-oxocyclohexane-1-carboxylate (502-1)

A mixture of ethyl (1S,3R,4R)-3-((tert-butoxycarbonyl)amino)-4-hydroxycyclohexane-1-carboxylate (3 g, 10.5 mmol) and IBX (6 g, 21.4 mmol) in acetonitrile (80 mL) was refluxed overnight. Additional IBX (3 g, 10.7 mmol) was added and stirred for another 5 h. The reaction was cooled and filtered. The filtrate was concentrated to obtain ethyl (1S,3R)-3-((tert-butoxycarbonyl)amino)-4-oxocyclohexane-1-carboxylate (3.2 g crude, quant) as a white solid. The crude product was used directly without further purification.

Step 2. Preparation of ethyl (1R,3S)-3-((tert-butoxycarbonyl)amino)-4-((4-(4-(trifluoromethyl)piperidin-1-yl)phenyl)amino) cyclohexane-1-carboxylate (502-2)

A solution of 4-(4-(trifluoromethyl)piperidin-1-yl)aniline (500 mg, 2.05 mmol), ethyl (1S,3R)-3-((tert-butoxycarbonyl)amino)-4-oxocyclohexane-1-carboxylate (800 mg, 2.81 mmol), and a drop of AcOH in DCE (10 mL) was stirred for 10 min before NaBH (OAc) 3 (600 mg, 2.84 mmol) was added and stirred at room temperature overnight. The reaction was quenched with sodium bicarbonate aqueous solution and extracted with DCM. The combined organic layers were washed with brine, dried over sodium sulfate and concentrated under vacuum. The residue was purified by silica gel Flash chromatography (PE/EA 90/10 to 70/30) to obtain 500 mg (47.5%) of the title compound as a grey solid. Mass (m/z): 514.5 [M+H]⁺.

Step 3 Preparation of (1R,3S)-3-((tert-butoxycarbonyl)amino)-4-((4-(4-(trifluoromethyl)piperidin-1-yl)phenyl)amino) cyclohexane-1-carboxylic acid (502-3)

A mixture of ethyl (1R,3S)-3-((tert-butoxycarbonyl)amino)-4-((4-(4-(trifluoromethyl)piperidin-1-yl)phenyl)amino) cyclohexane-1-carboxylate (300 mg. 0.584 mmol) and sodium hydroxide (300 mg, 7.5 mmol) in THF (1 mL), MeOH (1 mL) and water (0.5 mL) was stirred for 2 h. The reaction was concentrated under vacuum, adjusted pH to 5-6 with 2N HCl and extracted with ethyl acetate. The organic layer was dried over sodium sulfate and concentrated under vacuum to obtain the crude product (280 mg, 98.8%) as black syrup. Mass (m/z): 486.5 [M+H]⁺.

Step 4. Preparation of tert-butyl ((1S,5R)-5-carbamoyl-2-((4-(4-(trifluoromethyl)piperidin-1-yl)phenyl)amino)cyclohexyl)carba mate (502-4)

A mixture of (1R,3S)-3-((tert-butoxycarbonyl)amino)-4-((4-(4-(trifluoromethyl)piperidin-1-yl)phenyl)amino) cyclohexane-1-carboxylic acid (200 mg, 0.412 mmol) and CDI (150 mg, 2.42 mmol) in THF (2 mL) was refluxed overnight and added slowly to NH₃H₂O (5 mL). The resulting mixture was stirred for 10 min and then extracted with EtOAc twice. The combined organic layers were dried over sodium sulfate and concentrated under vacuum. The residue was purified by Prep-TLC (EtOAc) to obtain 180 mg (90.2%) of the product as a grey solid. Mass (m/z): 485.5 [M+H]⁺.

Step 5 Preparation of (1R,3S)-3-amino-4-((4-(4-(trifluoromethyl)piperidin-1-yl)phenyl)amino)cyclohexane-1-carbox amide (502)

A solution of tert-butyl ((1S,5R)-5-carbamoyl-2-((4-(4-(trifluoromethyl)piperidin-1-yl)phenyl)amino)cyclohexyl)carba mate (89 mg, 0.18 mmol) in 4N HCl in dioxane (1.5 mL) was stirred for 0.5 h at rt. The reaction was concentrated, basified with sodium bicarbonate and extracted with EA/EtOH (10/1) for 3 times. The combined organic layers were dried over sodium sulfate and concentrated under vacuum. The residue was purified by Prep-TLC (DCM/MeOH/NH₄OH 100/20/1, R_{f}-0.1) to obtain the title compound 502 (50.7 mg, 73.1%) as a white solid. ¹H NMR (400 MHZ, DMSO-d₆) δ 7.30-7.14 (m, 1H), 6.83-6.67 (m, 3H), 6.66-6.48 (m, 2H), 5.04-4.79 (m, 1H), 3.50-3.38 (m, 2H), 2.95-2.83 (m, 1H), 2.76-2.65 (m, 1H), 2.39-2.30 (m, 1H), 2.21-2.12 (m, 1H), 2.03-1.76 (m, 5H), 1.76-1.50 (m, 3H), 1.45-1.21 (m, 2H), 1.09-0.90 (m, 1H). Mass (m/z): 385.4 [M+H]⁺.

### Compound 503

### (2S, 4R)-4-(aminomethyl)-N1-(4-(4-(trifluoromethyl)piperidin-1-yl)phenyl)cyclohexane-1,2-dia mine bis(2,2,2-trifluoroacetate)

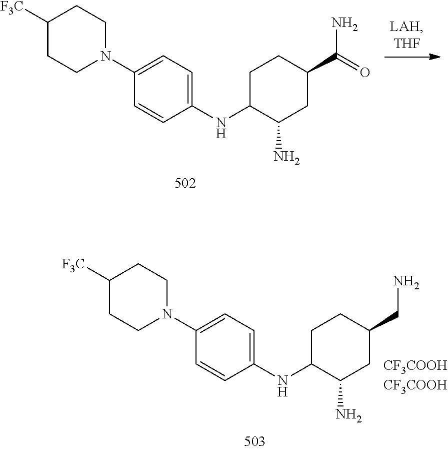 [SMILES: NC(=O)[C@@H]1CCC(Nc2ccc(N3CCC(C(F)(F)F)CC3)cc2)[C@@H](N)C1 ; NC[C@@H]1CCC(Nc2ccc(N3CCC(C(F)(F)F)CC3)cc2)[C@@H](N)C1]

A solution of (1R,3S)-3-amino-4-((4-(4-(trifluoromethyl)piperidin-1-yl)phenyl)amino)cyclohexane-1-carbox amide (30 mg, 0.08 mmol) and LiAlH₄ (1N in THF, 0.2 mL) in THF (2 mL) was refluxed for 2 h. The reaction was cooled and quenched with Na₂SO₄10H₂O, filtered and the filtrate was concentrated and purified by Prep-HPLC to obtain (2S,4R)-4-(aminomethyl)-N1-(4-(4-(trifluoromethyl)piperidin-1-yl)phenyl)cyclohexane-1,2-di amine bis(2,2,2-trifluoroacetate) 502 (5.2 mg, 10.8%) as a grey solid. ¹H NMR (400 MHZ, DMSO-d₆) δ 8.03-7.70 (m, 6H), 7.15-6.93 (m, 2H), 6.67 (d, J=8.5 Hz, 2H), 3.65-3.47 (m, 6H), 3.03-2.85 (m, 1H), 2.85-2.65 (m, 2H), 2.15-1.89 (m, 4H), 1.84-1.50 (m, 4H), 1.30-0.93 (m, 2H). Mass (m/z): 371.3 [M+H]⁺.

### Compound 504

### N2-(6-(2,6-dimethylmorpholino)-2-methylpyridin-3-yl)bicyclo[2.2.1]heptane-2,5-diamine

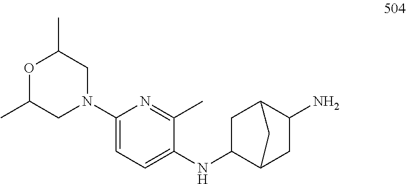 [SMILES: Cc1nc(N2CC(C)OC(C)C2)ccc1NC1CC2CC1CC2N]

The title compound 504 (14.8 mg) was prepared in a total yield of 44.8% as a white solid according to the procedure for compound 24. ¹H NMR (400 MHZ, DMSO-d₆) δ 6.94 (d, J=8.8 Hz, 1H), 6.55 (d, J=8.8 Hz, 1H), 3.95-3.50 (m, 5H), 3.17 (m, 1H), 2.46-2.10 (m, 7H), 2.02-1.76 (m, 3H), 1.67-1.45 (m, 2H), 1.36 (m, 1H), 1.12 (d, J=6.0 Hz, 6H). Mass (m/z): 331.2 [M+H]⁺.

### Compound 505

### N2-(4-(4-(2,2,2-trifluoroethyl) piperazin-1-yl)phenyl)spiro[3.3]heptane-2,6-diamine

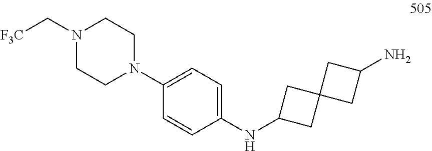 [SMILES: NC1CC2(C1)CC(Nc1ccc(N3CCN(CC(F)(F)F)CC3)cc1)C2]

The title compound 505 (25.1 mg) was prepared in a total yield of 68.0% as a gray solid according to the procedure for compound 24. ¹H NMR (400 MHZ, DMSO-d₆) δ 6.85-6.68 (m, 2H), 6.57-6.44 (m, 2H), 3.37 (s, 2H), 3.27-3.15 (m, 2H), 2.83-2.67 (m, 6H), 2.05-1.98 (m, 2H), 1.89-1.77 (m, 2H), 1.52-1.24 (m, 4H), 1.05-0.93 (m, 2H). Mass (m/z): 369.2 [M+H]⁺.

### Compound 506

### tert-butyl ((1r,4r)-4-(((4-(6-oxa-3-azabicyclo[3.1.1]heptan-3-yl)-3-fluorophenyl)amino)methyl)cyclohexyl) carbamate

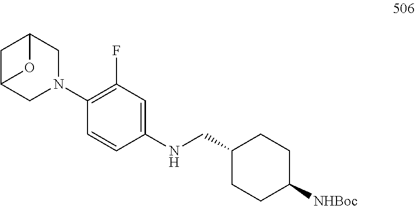 [SMILES: CC(C)(C)OC(=O)N[C@H]1CC[C@H](CNc2ccc(N3CC4CC(C3)O4)c(F)c2)CC1]

The title compound 506 (14.8 mg) was prepared in a total yield of 14.7% as a white solid from 4-(6-oxa-3-azabicyclo[3.1.1]heptan-3-yl)-3-fluoroaniline (50 mg, 0.240 mmol), tert-butyl ((1r,4r)-4-formylcyclohexyl)carbamate (82 mg, 0.360 mmol), AcOH (0.1 mL), NaBH (OAc) 3 (102 mg, 0.480 mmol) and DCE (10 mL) according to the procedure for 24-1. ¹H NMR (400 MHz, DMSO-d₆) δ 6.87 (dd, J=10.4, 8.8 Hz, 1H), 6.69 (d, J=8.0 Hz, 1H), 6.41-6.25 (m, 2H), 5.46 (t, J=5.6 Hz, 1H), 4.53 (d, J=6.0 Hz, 2H), 3.38 (d, J=11.2 Hz, 2H), 3.31 (s, 2H), 3.17 (d, J=8.0 Hz, 1H), 2.99 (dt, J=8.0, 6.4 Hz, 1H), 2.78 (t, J=6.0 Hz, 2H), 2.23 (d, J=8.0 Hz, 1H), 1.80 (d, J=11.6 Hz, 4H), 1.37 (s, 9H), 1.16-1.05 (m, 2H), 1.01-0.89 (m, 2H). Mass (m/z): 420.3 [M+H]⁺.

### Compound 507

### N2-(2-methyl-6-(8-oxa-2-azaspiro[4.5]decan-2-yl)pyridin-3-yl)spiro[3.3]heptane-2,6-diamine

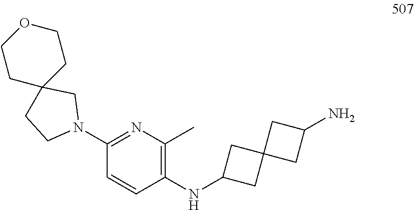 [SMILES: Cc1nc(N2CCC3(CCOCC3)C2)ccc1NC1CC2(CC(N)C2)C1]

The title compound 507 (12.7 mg) was prepared in a total yield of 29.5% as a yellow solid from tert-butyl (6-((2-methyl-6-(8-oxa-2-azaspiro[4.5]decan-2-yl)pyridin-3-yl)amino)spiro[3.3]heptan-2-yl)ca rbamate (55 mg, 0.121 mmol), TFA (1 mL), and DCM (10 mL) according to the procedure for 24. ¹H NMR (400 MHZ, DMSO-d₆) δ 8.52 (s, 1H), 8.42 (s, 1H), 3.71-3.49 (m, 4H), 3.43 (s, 1H), 3.32 (s, 2H), 3.21-3.14 (m, 1H), 3.12-3.07 (m, 1H), 2.45 (d, J=8.0 Hz, 2H), 2.42-2.30 (m, 2H), 2.19 (d, J=8.5 Hz, 1H), 1.94 (d, J=20.0 Hz, 1H), 1.86 (t, J=7.2 Hz, 1H), 1.53 (d, J=5.2 Hz, 2H). Mass (m/z): 357.3 [M+H]⁺.

### Compound 508

### N2-(6-((4aR,8aR)-hexahydro-2H-pyrano [3,2-c]pyridin-6 (5H)-yl)-2-methylpyridin-3-yl)spiro[3. 3]heptane-2,6-diamine

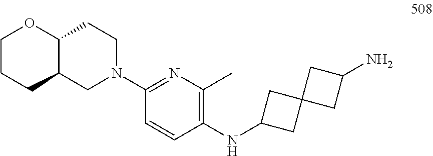 [SMILES: Cc1nc(N2CC[C@H]3OCCC[C@@H]3C2)ccc1NC1CC2(CC(N)C2)C1]

The title compound 508 (48.8 mg) was prepared in a total yield of 86.7% as a yellow solid from tert-butyl (6-((6-((4aR,8aR)-hexahydro-2H-pyrano [3,2-c]pyridin-6 (5H)-yl)-2-methylpyridin-3-yl)amino) spiro [3.3]heptan-2-yl)carbamate (72 mg, 0.158 mmol), TFA (1 mL), and DCM (10 mL) according to the procedure for 24. ¹H NMR (400 MHZ, DMSO-d₆) δ 8.44 (s, 3H), 6.82-6.68 (m, 1H), 6.54 (d, J=8.8 Hz, 1H), 4.60 (s, 1H), 4.07 (d, J=12.8 Hz, 1H), 3.88 (dd, J=16.8, 11.4 Hz, 2H), 3.71-3.46 (m, 3H), 3.00 (td, J=10.4, 4.2 Hz, 1H), 2.61 (t, J=13.2 Hz, 2H), 2.41-2.30 (m, 2H), 2.22 (td, J=15.6, 13.2, 8.0 Hz, 7H), 1.93 (d, J=11.2 Hz, 2H), 1.73 (dd, J=32.0, 12.0 Hz, 2H), 1.57 (qd, J=7.6, 5.2, 3.2 Hz, 2H), 1.50-1.28 (m, 2H), 1.26-1.11 (m, 1H). Mass (m/z): 357.3 [M+H]⁺.

### Compound 509

### N2-(2-methyl-6-(1-oxa-9-azaspiro [5.5]undecan-9-yl)pyridin-3-yl)spiro[3.3]heptane-2,6-diamine

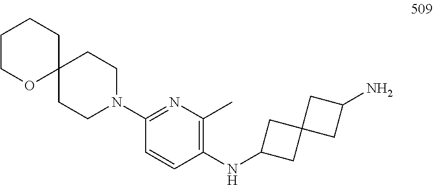 [SMILES: Cc1nc(N2CCC3(CCCCO3)CC2)ccc1NC1CC2(CC(N)C2)C1]

The title compound 509 (48.4 mg) was prepared in a total yield of 89.0% as a yellow solid from tert-butyl (6-((2-methyl-6-(1-oxa-9-azaspiro [5.5]undecan-9-yl)pyridin-3-yl)amino)spiro[3.3]heptan-2-yl) carbamate (69 mg, 0.147 mmol), TFA (1 mL), and DCM (10 mL) according to the procedure for 24. ¹H NMR (400 MHZ, DMSO-d₆) δ 8.47 (d, J=39.6 Hz, 6H), 7.11 (s, 1H), 6.43 (s, 1H), 3.71-3.50 (m, 8H), 3.43 (s, 2H), 3.32 (s, 1H), 3.19-3.14 (m, 2H), 3.11-3.06 (m, 2H), 2.45 (d, J=8.0 Hz, 5H), 2.40-2.30 (m, 4H), 2.22 (dd, J=21.6, 8.4 Hz, 3H), 1.96 (s, 1H), 1.86 (t, J=7.2 Hz, 2H), 1.53 (d, J=5.2 Hz, 4H). Mass (m/z): 371.3 [M+H]⁺.

### Compound 510

### N-(((1r,4r)-4-aminocyclohexyl)methyl)-6-(4-methoxy-4-(trifluoromethyl)piperidin-1-yl)-2-methylpyridin-3-amine

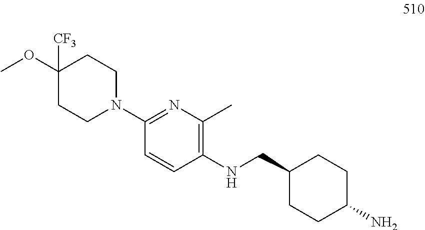 [SMILES: COC1(C(F)(F)F)CCN(c2ccc(NC[C@H]3CC[C@H](N)CC3)c(C)n2)CC1]

The title compound 510 (24.8 mg) was prepared in a total yield of 35.2% as a white solid from tert-butyl ((1r,4r)-4-(((6-(4-methoxy-4-(trifluoromethyl)piperidin-1-yl)-2-methylpyridin-3-yl)amino) met hyl)cyclohexyl)carbamate (88 mg, 0.176 mmol), TFA (1 mL) and DCM (10 mL) according to the procedure for 24. ¹H NMR (400 MHZ, DMSO-d₆) δ 8.45-8.18 (m, 3H), 7.20-6.71 (m, 2H), 4.83 (s, 1H), 3.52 (s, 2H), 3.12 (s, 3H), 2.88 (d, J=6.8 Hz, 3H), 2.32 (s, 3H), 2.05-1.95 (m, 2H), 1.87 (d, J=12.8 Hz, 2H), 1.74 (d, J=13.2 Hz, 2H), 1.53 (s, 2H), 1.42-1.27 (m, 2H), 1.00 (dd, J=13.6, 10.4 Hz, 2H). Mass (m/z): 401.3 [M+H]⁺.

### Compound 511

### N-(((1r,4R)-4-aminocyclohexyl)methyl)-6-((2S,6R)-2,6-dimethylmorpholino)-2-methoxypyridin-3-amine

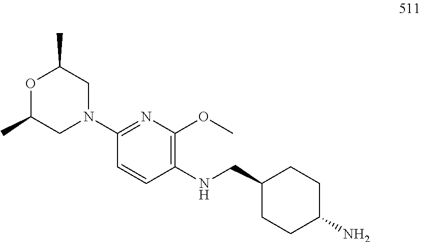 [SMILES: COc1nc(N2C[C@@H](C)O[C@@H](C)C2)ccc1NC[C@H]1CC[C@H](N)CC1]

The title compound 511 (14.5 mg) was prepared in a total yield of 28.3% as a purple solid from tert-butyl ((1R,4r)-4-(((6-((2S,6R)-2,6-dimethylmorpholino)-2-methoxypyridin-3-yl)amino)methyl)cyclo hexyl)carbamate (66 mg, 0.147 mmol), TFA (1 mL), and DCM (10 mL) according to the procedure for 24. ¹H NMR (400 MHZ, DMSO-d₆) δ 8.13 (s, 2H), 6.75 (d, J=8.0 Hz, 1H), 6.16 (d, J=8.0 Hz, 1H), 4.33 (s, 1H), 3.99 (td, J=6.4, 3.2 Hz, 1H), 3.79 (d, J=2.4 Hz, 1H), 3.63 (ddd, J=10.4, 6.4, 2.4 Hz, 2H), 3.27 (dd, J=12.0, 3.2 Hz, 1H), 2.96-2.79 (m, 4H), 2.23-2.12 (m, 3H), 1.96 (d, J=12.0 Hz, 2H), 1.81 (d, J=12.8 Hz, 2H), 1.47 (s, 1H), 1.29 (d, J=13.6 Hz, 2H), 1.17 (s, 1H), 1.17-1.12 (m, 6H), 0.97 (q, J=12.8, 12.4 Hz, 2H). Mass (m/z): 349.3 [M+H]⁺.

### Compound 512

### N-((6-aminospiro [3.3]heptan-2-yl)methyl)-6-(2,6-dimethylmorpholino)-2-methylpyridin-3-amine

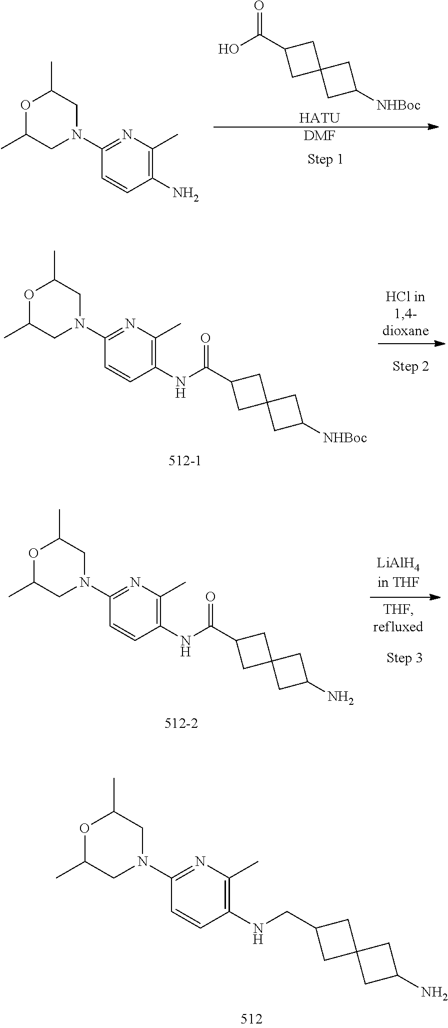

Step 1. Preparation of tert-butyl (6-((6-(2,6-dimethylmorpholino)-2-methylpyridin-3-yl) carbamoyl)spiro[3.3]heptan-2-yl)carbamate (512-1)

The title compound 512-1 (360 mg) was prepared in a total yield of 78.6% as a yellow solid from 6-((tert-butoxycarbonyl)amino)spiro[3.3]heptane-2-carboxylic acid (338 mg, 1.5 mmol), 6-(2,6-dimethylmorpholino)-2-methylpyridin-3-amine (219 mg, 1.0 mmol), DIEA (387 mg, mmol) and HATU (456 mg, 1.2 mmol) according to the procedure for 403. Mass (m/z): 381.3 [M+H]⁺.

Step 2. Preparation of 6-amino-N-(6-(2,6-dimethylmorpholino)-2-methylpyridin-3-yl)spiro[3.3]heptane-2-carboxamide (512-2)

The title compound 512-2 (260 mg) was prepared in a total yield of 92.5% as a light yellow solid from tert-butyl (6-((6-(2,6-dimethylmorpholino)-2-methylpyridin-3-yl) carbamoyl)spiro[3.3]heptan-2-yl) carba mate (360 mg, 0.79 mmol) and 5.0 mL of HCl in 1,4 dioxane according to the procedure for compound 37. Mass (m/z): 359.2 [M+H]⁺.

Step 3. Preparation of N-((6-aminospiro [3.3]heptan-2-yl)methyl)-6-(2,6-dimethylmorpholino)-2-methylpyridin-3-ami ne (512)

The title compound 512 (9.4 mg) was prepared in a total yield of 4.9% as a purple solid from 6-amino-N-(6-(2,6-dimethylmorpholino)-2-methylpyridin-3-yl)spiro[3.3]heptane-2-carboxami de (200 mg, 0.56 mmol) and 1.16 mL of LiAlH₄ in THF (2.4 M, 2.78 mmol) according to the procedure for compound 23. ¹H NMR (400 MHZ, DMSO-d₆) δ 7.87 (s, 3H), 6.82 (d, J=8.8 Hz, 1H), 6.50 (d, J=8.7 Hz, 1H), 4.28 (t, J=5.8 Hz, 1H), 3.87-3.79 (m, 2H), 3.67-3.47 (m, 4H), 2.97 (t, J=6.4 Hz, 2H), 2.39 (s, 1H), 2.22-2.01 (m, 9H), 1.81-1.68 (m, 2H), 1.14 (d, J=6.2 Hz, 6H). Mass (m/z): 345.2 [M+H]⁺.

### Compound 513

### N-(((1r,4r)-4-aminocyclohexyl)methyl)-3-fluoro-4-(2,2,6,6-tetramethylmorpholino)aniline

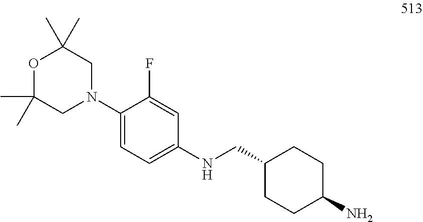 [SMILES: CC1(C)CN(c2ccc(NC[C@H]3CC[C@H](N)CC3)cc2F)CC(C)(C)O1]

The title compound 513 (22.0 mg) was prepared in a total yield of 40.2% as a white solid according to the procedure for compound 24. ¹H NMR (400 MHZ, DMSO-d₆) δ 8.04 (s, 3H), 6.79 (dd, J=10.0, 8.6 Hz, 1H), 6.35 (dd, J=14.7, 2.5 Hz, 1H), 6.29 (dd, J=8.6, 2.6 Hz, 1H), 2.96-2.85 (m, 1H), 2.80 (t, J=6.1 Hz, 2H), 2.01-1.93 (m, 2H), 1.88-1.78 (m, 2H), 1.49-1.39 (m, 1H), 1.34-1.24 (m, 2H), 1.21 (s, 12H), 1.04-0.91 (m, 2H). Mass (m/z): 364.2 [M+H]⁺.

### Compound 514

### N-(((1r,4r)-4-aminocyclohexyl)methyl)-2-methyl-6-(2,2,6,6-tetramethylmorpholino)pyridin-3-amine

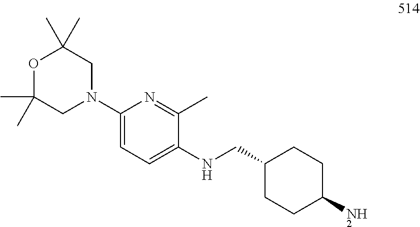 [SMILES: Cc1nc(N2CC(C)(C)OC(C)(C)C2)ccc1NC[C@H]1CC[C@H](N)CC1]

The title compound 514 (13.0 mg) was prepared in a total yield of 24.1% as a yellow solid according to the procedure for compound 24. ¹H NMR (400 MHZ, DMSO-d₆) δ 8.15 (s, 3H), 7.10-6.76 (m, 1H), 6.69-6.37 (m, 1H), 4.63-4.32 (m, 1H), 3.23-3.07 (m, 3H), 2.97-2.72 (m, 3H), 2.27 (s, 3H), 2.03-1.80 (m, 4H), 1.63-1.44 (m, 1H), 1.36-1.27 (m, 2H), 1.18 (s, 12H), 1.06-0.93 (m, 2H). Mass (m/z): 361.3 [M+H]⁺.

### Compound 515

### N2-(2-methyl-6-(2,2,6,6-tetramethylmorpholino)pyridin-3-yl)spiro[3.3]heptane-2,6-diamine

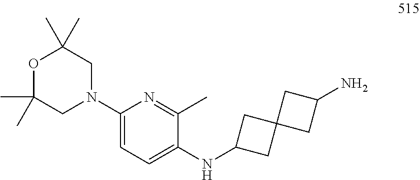 [SMILES: Cc1nc(N2CC(C)(C)OC(C)(C)C2)ccc1NC1CC2(CC(N)C2)C1]

The title compound 515 (15.2 mg) was prepared in a total yield of 28.3% as a white solid according to the procedure for compound 24. ¹H NMR (400 MHZ, DMSO-d₆) δ 8.13 (s, 3H), 6.76 (s, 1H), 6.53 (d, J=8.1 Hz, 1H), 4.61-4.45 (m, 1H), 3.61 (d, J=35.7 Hz, 2H), 3.14-3.06 (m, 4H), 2.45-2.34 (m, 2H), 2.27-2.05 (m, 7H), 1.98-1.87 (m, 7H), 1.18 (s, 12H). Mass (m/z): 359.2 [M+H]⁺.

### Compound 516

### N-(((1s,4r)-4-aminoadamantan-1-yl)methyl)-6-(2,6-dimethylmorpholino)-2-methylpyridin-3-amine

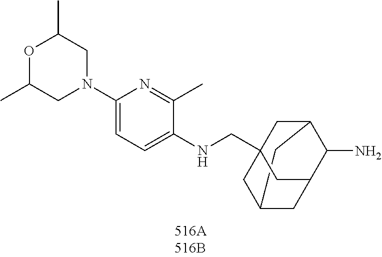 [SMILES: Cc1nc(N2CC(C)OC(C)C2)ccc1NCC12CC3CC(C1)C(N)C(C3)C2]

The title compound 516A (Rt=8.59 min; 5.4 mg) was prepared in a three-step total yield of 7.2% as a white solid and compound 516B (Rt=9.96 min; 1.4 mg) was prepared in a three-step total yield of 2.5% as a white solid ¹H NMR from 6-(2,6-dimethylmorpholino)-2-methylpyridin-3-amine (300 mg, 1.36 mmol) and 4-oxoadamantane-1-carboxylic acid (316 mg, 1.63 mmol) according to the procedure for 20. The target products were purified by Prep-HPLC (Column: X Select-CSH-Prep 5 μm OBD, 19*150 mm; ACN/water (0.5% TFA)=0%-25%-95%-95%-10%, 0 min-13 min-13.5 min-14.5 min-16.0 min). 516A: ¹H NMR (400 MHz, DMSO-d₆) δ 6.93 (d, J=8.8 Hz, 1H), 6.50 (d, J=8.7 Hz, 1H), 3.97 (t, J=6.5 Hz, 1H), 3.88-3.79 (m, 2H), 3.62 (ddd, J=10.3, 6.3, 2.4 Hz, 2H), 2.84 (s, 1H), 2.71 (d, J=6.0 Hz, 2H), 2.15 (dd, J=12.3, 10.3 Hz, 3H), 2.03 (d, J=12.6 Hz, 2H), 1.83 (d, J=10.4 Hz, 1H), 1.67 (s, 2H), 1.54 (dd, J=24.3, 11.6 Hz, 6H), 1.32-1.23 (m, 3H), 1.14 (d, J=6.2 Hz, 6H). Mass (m/z): 385.6 [M+H]⁺. 516B: ¹H NMR (400 MHZ, DMSO-d₆) δ 6.93 (d, J=8.8 Hz, 1H), 6.50 (d, J=8.5 Hz, 1H), 3.97 (t, J=6.5 Hz, 1H), 3.89-3.79 (m, 2H), 3.68 (ddd, J=10.3, 6.3, 2.4 Hz, 2H), 2.86 (s, 1H), 2.71 (d, J=6.0 Hz, 2H), 2.15 (dd, J=12.3, 10.3 Hz, 3H), 2.03 (d, J=12.7 Hz, 2H), 1.83 (d, J=10.9 Hz, 1H), 1.67 (s, 2H), 1.52 (dd, J=24.3, 11.6 Hz, 6H), 1.38-1.26 (m, 2H), 1.18 (d, J=6.2 Hz, 6H). Mass (m/z): 385.6 [M+H]⁺.

### Compound 517

### N4-(2-methyl-6-morpholinopyridin-3-yl)adamantane-1,4-diamine

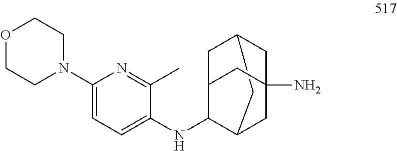 [SMILES: Cc1nc(N2CCOCC2)ccc1NC1C2CC3CC1CC(N)(C3)C2]

The title compound 517 (38.4 mg) was prepared in a yield of 30.5% as a white powder from 2-methyl-6-morpholinopyridin-3-amine (70 mg, 0.36 mmol) and tert-butyl (4-oxoadamantan-1-yl)carbamate (114 mg, 0.44 mmol) according to the procedure for 20. ¹H NMR (400 MHZ, DMSO-d₆) δ 8.26 (s, 2H), 8.18 (s, 1H), 6.99 (s, 1H), 6.59 (s, 1H), 4.00 (s, 1H), 3.69 (t, J=4.7 Hz, 5H), 3.23 (s, 2H), 2.34 (dt, J=4.0, 2.1 Hz, 2H), 2.22-2.00 (m, 5H), 1.99-1.86 (m, 4H), 1.86-1.73 (m, 3H), 1.62 (dd, J=43.0, 12.0 Hz, 1H), 1.36 (d, J=12.5 Hz, 2H). Mass (m/z): 343.4 [M+H]⁺.

### Compound 518

### N4-(2-methyl-6-(2-methylmorpholino)pyridin-3-yl)adamantane-1,4-diamine

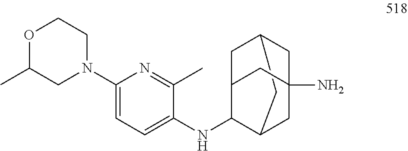 [SMILES: Cc1nc(N2CCOC(C)C2)ccc1NC1C2CC3CC1CC(N)(C3)C2]

The title compound 518 (31.0 mg) was prepared in a yield of 27.7% as a white powder from 2-methyl-6-(2-methylmorpholino)pyridin-3-amine (70 mg, 0.34 mmol) and tert-butyl (4-oxoadamantan-1-yl)carbamate (108 mg, 0.41 mmol) according to the procedure for 20. ¹H NMR (400 MHZ, DMSO-d₆) δ 8.27 (s, 2H), 8.18 (s, 1H), 7.03 (s, 1H), 6.61 (s, 1H), 3.95-3.83 (m, 2H), 3.76 (d, J=12.4 Hz, 1H), 3.54 (t, J=11.3 Hz, 2H), 3.36 (s, 3H), 2.35 (s, 2H), 2.22-1.70 (m, 11H), 1.60 (dd, J=41.7, 12.1 Hz, 1H), 1.34 (d, J=12.4 Hz, 1H), 1.13 (d, J=6.2 Hz, 3H). Mass (m/z): 357.4 [M+H]⁺.

### Compound 519

### N4-(6-(2,6-dimethylmorpholino)-5-fluoro-2-methylpyridin-3-yl)adamantane-1,4-diamine

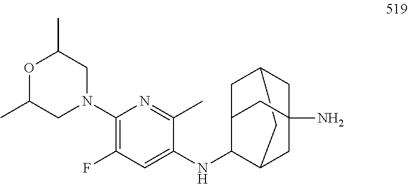 [SMILES: Cc1nc(N2CC(C)OC(C)C2)c(F)cc1NC1C2CC3CC1CC(N)(C3)C2]

The title compound 519 (15.0 mg) was prepared in a yield of 18.5% as a white powder from 6-(2,6-dimethylmorpholino)-5-fluoro-2-methylpyridin-3-amine (50 mg, 0.21 mmol) and tert-butyl (4-oxoadamantan-1-yl)carbamate (67 mg, 0.25 mmol) according to the procedure for 20. ¹H NMR (400 MHZ, DMSO-d₆) δ 8.13 (s, 3H), 6.87 (dd, J=14.6, 8.5 Hz, 1H), 4.31-4.19 (m, 1H), 3.68 (ddd, J=10.4, 6.2, 2.1 Hz, 2H), 3.38 (t, J=13.2 Hz, 3H), 2.26 (d, J=1.1 Hz, 2H), 2.19-2.05 (m, 3H), 2.04-1.92 (m, 3H), 1.87 (d, J=11.6 Hz, 1H), 1.79 (d, J=18.7 Hz, 3H), 1.65 (d, J=12.6 Hz, 1H), 1.56 (d, J=11.2 Hz, 1H), 1.35 (d, J=12.8 Hz, 1H), 1.09 (d, J=6.2 Hz, 6H). Mass (m/z): 389.5 [M+H]⁺.

### Compound 520

### N-(5-(aminomethyl) adamantan-2-yl)-2-methyl-6-(2-methylmorpholino)pyridin-3-amine

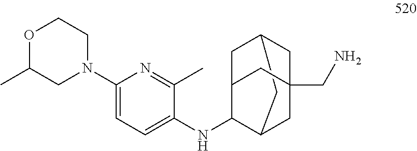 [SMILES: Cc1nc(N2CCOC(C)C2)ccc1NC1C2CC3CC1CC(CN)(C3)C2]

The title compound 520 (50.3 mg) was prepared in a two-step overall yield of 43.2% as a white powder from 2-methyl-6-(2-methylmorpholino)pyridin-3-amine (200 mg, 1.03 mmol) and 4-oxoadamantane-1-carboxylic acid (301 mg, 1.51 mmol) according to the procedure for 84. 1¹H NMR (400 MHZ, DMSO-d₆) δ 8.09 (s, 3H), 6.90 (s, 1H), 6.57 (s, 1H), 3.88 (d, J=11.5 Hz, 3H), 3.76 (d, J=12.3 Hz, 1H), 3.64-3.46 (m, 3H), 2.61 (d, J=13.9 Hz, 2H), 2.33 (d, J=10.8 Hz, 4H), 1.95 (d, J=29.9 Hz, 5H), 1.80-1.67 (m, 2H), 1.64 (d, J=2.9 Hz, 3H), 1.58-1.47 (m, 3H), 1.34 (dd, J=26.2, 12.2 Hz, 3H), 1.14 (d, J=6.2 Hz, 3H). Mass (m/z): 371.5 [M+H]⁺.

### Compound 521

### tert-butyl (6-((2-methyl-6-(tetrahydro-1H-furo[3,4-c]pyrrol-5 (3H)-yl)pyridin-3-yl)amino)spiro[3.3]hepta n-2-yl)carbamate

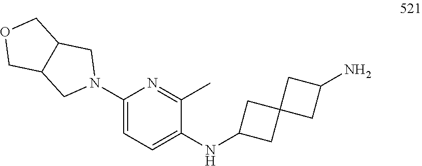 [SMILES: Cc1nc(N2CC3COCC3C2)ccc1NC1CC2(CC(N)C2)C1]

The title compound 521 (6.8 mg, 11.6% yield) as a light brown solid was prepared from tert-butyl (6-((2-methyl-6-(tetrahydro-1H-furo[3,4-c]pyrrol-5 (3H)-yl)pyridin-3-yl)amino)spiro[3.3]hepta n-2-yl)carbamate (70 mg, 0.163 mmol), DCM (3 mL) and TFA (1 mL) and purified by prep-HPLC (CH₃CN/0.05% FA in water) according to the procedure for 24. ¹H NMR (400 MHz, DMSO-d₆) δ 6.87 (d, J=8.7 Hz, 1H), 6.32 (d, J=8.7 Hz, 1H), 3.96-3.91 (m, 2H), 3.74-3.63 (m, 1H), 3.60 (dd, J=8.8, 3.9 Hz, 2H), 3.49-3.33 (m, 3H), 3.28-3.24 (m, 2H), 3.02-2.95 (m, 2H), 2.57-2.41 (m, 2H), 2.40-2.36 (m 1H), 2.31-2.23 (m, 4H), 2.02-1.86 (m, 4H). MS (m/z): 329.2 [M+H]⁺.

### Compound 522

### N-(((1r,4r)-4-aminocyclohexyl)methyl)-4-(3,5-dimethylpiperidin-1-yl)aniline

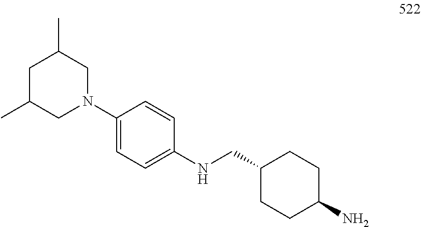 [SMILES: CC1CC(C)CN(c2ccc(NC[C@H]3CC[C@H](N)CC3)cc2)C1]

The title compound 522 (25.3 mg, 9.4%) as a white solid was prepared from tert-butyl ((1r,4r)-4-(((4-(3,5-dimethylpiperidin-1-yl)phenyl)amino)methyl)cyclohexyl)carbamate (350 mg, 0.8401 mmol), DCM (10 mL) and TFA (1 mL) according to the procedure for 24. ¹H NMR (400 MHz, CD₃OD) δ 6.91-6.84 (m, 2H), 6.62-6.58 (m, 2H), 3.29-3.26 (m, 1H), 2.88 (d, J=6.7 Hz, 2H), 2.55 (ddd, J=14.4, 7.3, 3.5 Hz, 1H), 2.06 (t, J=11.3 Hz, 2H), 1.90-1.76 (m, 7H), 1.52 (brs, 1H), 1.16-0.94 (m, 5H), 0.93 (d, J=6.4 Hz, 6H), 0.67-0.57 (m, 1H). Mass (m/z): 316.3 [M+H]⁺.

### Compound 523

### N2-(6-(3-oxa-6-azabicyclo[3.1.1]heptan-6-yl)-2-methylpyridin-3-yl)spiro[3.3]heptane-2, 6-diamine

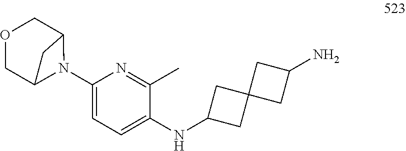 [SMILES: Cc1nc(N2C3COCC2C3)ccc1NC1CC2(CC(N)C2)C1]

The title compound 523 (1.5 mg, 1.12% yield) as a white solid was prepared from tert-butyl (6-((6-(3-oxa-6-azabicyclo[3.1.1]heptan-6-yl)-2-methylpyridin-3-yl)amino)spiro[3.3]heptan-2-yl)carbamate (170 mg, 0.4091 mmol), DCM (10 mL) and TFA (1 mL) according to the procedure for 24. ¹H NMR (400 MHZ, CD₃OD) δ 6.91 (d, J=8.5 Hz, 1H), 6.34 (d, J=8.5 Hz, 1H), 4.28 (m, 4H), 3.70 (m, 4H), 2.71 (d, J=6.7 Hz, 1H), 2.52-2.34 (m, 3H), 2.26 (s, 3H), 2.03-1.59 (m, 6H). Mass (m/z): 315.2 [M+H]⁺.

### Compound 524

### N2-(6-(6-oxa-3-azabicyclo[3.1.1]heptan-3-yl)-2-methylpyridin-3-yl)spiro[3.3]heptane-2,6-diamine

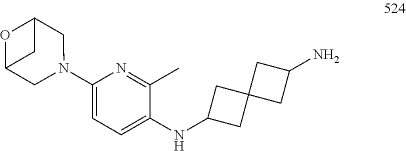 [SMILES: Cc1nc(N2CC3CC(C2)O3)ccc1NC1CC2(CC(N)C2)C1]

The title compound 524 (6.9 mg, 9.1% yield) as yellow oil was prepared from tert-butyl (6-((6-(6-oxa-3-azabicyclo[3.1.1]heptan-3-yl)-2-methylpyridin-3-yl)amino)spiro[3.3]heptan-2-yl)carbamate (0.1 g, 0.24 mmol), DCM (5 mL) and TFA (1 mL) according to the procedure for 24. ¹H NMR (400 MHZ, CD₃OD) δ 6.93 (d, J=8.8 Hz, 1H), 6.39 (d, J=8.8 Hz, 1H), 4.72 (d, J=6 Hz, 2H), 3.75-3.63 (m, 3H), 3.50 (d, J=12 Hz, 2H), 3.33 (t, J=7.4 Hz, 1H), 3.18-3.20 (m, 1H), 2.55-2.15 (m, 7H), 2.01-1.81 (m, 5H). Mass (m/z): 314.9 [M+H]⁺.

### Compound 525

### N-(((1r,4r)-4-aminocyclohexyl)methyl)-4-(3,4,5-trimethylpiperazin-1-yl)aniline

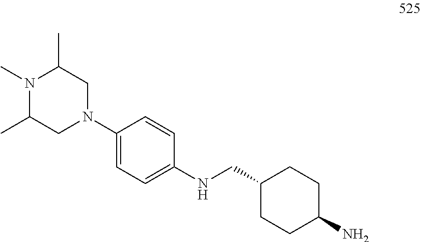 [SMILES: CC1CN(c2ccc(NC[C@H]3CC[C@H](N)CC3)cc2)CC(C)N1C]

The title compound 525 (40.8 mg, 44.3% yield) as a yellow solid was prepared from tert-butyl ((1r,4r)-4-(((4-(3,4,5-trimethylpiperazin-1-yl)phenyl)amino)methyl)cyclohexyl)carbamate (0.12 g, 0.28 mmol), DCM (5 mL) and TFA (1 mL) according to the procedure for 24. ¹H NMR (400 MHz, CD₃OD) δ 6.86-6.76 (m, 2H), 6.62-6.50 (m, 2H), 3.29-3.22 (m, 2H), 2.86 (d, J=6.6 Hz, 2H), 2.58-2.56 (m, 1H), 2.45-2.37 (m, 4H), 2.31 (s, 3H), 1.94-1.82 (m, 4H), 1.51 (s, 1H), 1.20-0.97 (m, 10H). Mass (m/z): 330.9 [M+H]⁺.

### Compound 526

### N-(3-((1r,4s)-4-aminocyclohexyl)propyl)-6-(2,6-dimethylmorpholino)-2-methyl pyridin-3-amine

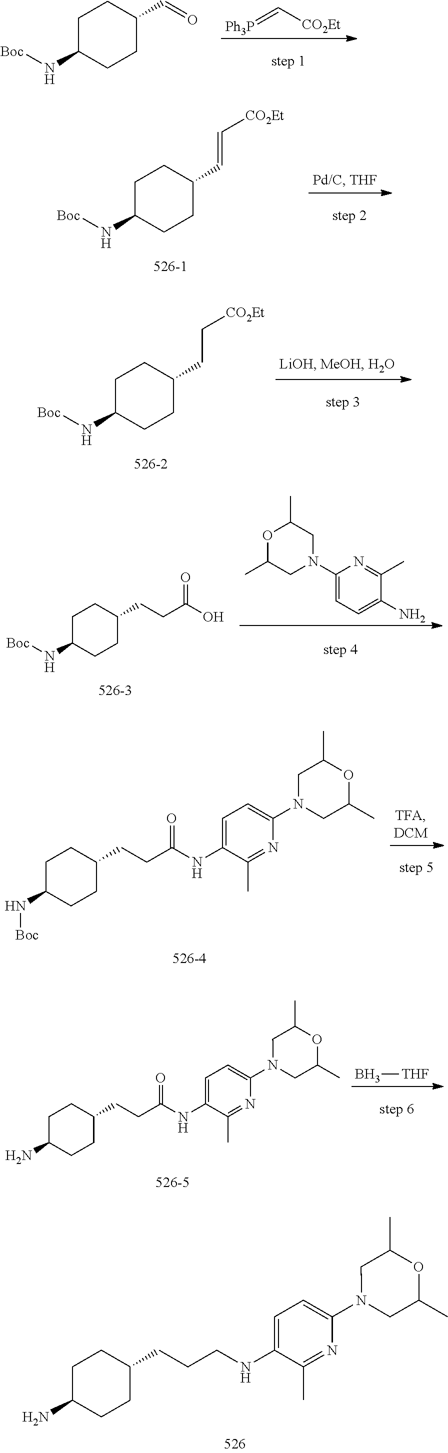

Step 1. Preparation of ethyl (E)-3-((1r,4r)-4-((tert-butoxycarbonyl)amino)cyclohexyl)acrylate (526-1)

A mixture of tert-butyl ((1r,4r)-4-formylcyclohexyl)carbamate (3 g, 13.1 mmol), ethyl 2-(triphenyl-15-phosphaneylidene) acetate (4.56 g, 13.1 mmol), NaH (0.38 g, 15.7 mmol) and THF (20 mL) was stirred at 25° C. for 2 hrs. The mixture was diluted with EA (20 mL) and washed with water (20 mL×3). The organic phase was concentrated to give 526-1 as a yellow solid (4 g, 72%). Mass (m/z): 319.8 [M+Na]⁺.

Step 2. Preparation of ethyl 3-((1r,4r)-4-((tert-butoxycarbonyl)amino)cyclohex yl)propanoate (526-2)

To a solution of 526-1 (4 g, 13.4 mmol) in THF (30 mL) was added wet Pd/C (1 g) under H₂ at 25° C. The reaction was stirred at 25° C. for 3 hrs. The mixture was filtered and concentrated to give 526-2 as pale oil (2 g, 45%). Mass (m/z): 199.9 [M+H]⁺.

Step 3. Preparation of 3-((1r,4r)-4-((tert-butoxycarbonyl)amino)cyclohexyl) propanoic acid (526-3)

To a solution of 526-2 (2 g, 6.7 mmol) in MeOH (20 mL) was added LiOH (0.84 g, 20.1 mmol) at 25° C. The reaction was stirred at 25° C. for 2 hrs. TLC showed SM was consumed. The mixture was poured into water and filtered to give 526-3 as a white solid (2 g, 100%).

Step 4. Preparation of tert-butyl ((1r,4r)-4-(3-((6-(2,6-dimethylmorpholino)-2-methylpyridin-3-yl)amino)-3-oxopropyl)cyclohe xyl)carbamate (526-4)

To a solution of 526-3 (0.2 g, 0.7 mmol), 6-(2,6-dimethylmorpholino)-2-methylpyridin-3-amine (0.15 g, 0.7 mmol) and HATU (0.27 g, 0.7 mmol) in DMF (5 mL) was added DIEA (0.27 g, 2.1 mmol). The reaction was stirred at 25° C. for 16 hrs. The mixture was poured into water and filtered to give 526-4 as a white solid (0.3 g, 86%). Mass (m/z): 474.8 [M+H]⁺.

Step 5. Preparation of 3-((1r,4r)-4-aminocyclohexyl)-N-(6-(2,6-dimethylmorpholi no)-2-methylpyridin-3-yl) propanamide (526-5)

To a solution of 526-4 (0.3 g, 0.6 mmol) in DCM (5 mL) was added TFA (1 mL). The reaction was stirred at 25° C. for 2 hrs. The pH of the mixture was adjusted to 8 by aq. Na₂CO₃ and extracted with DCM. The organic phase was concentrated to give 526-5 as a brown solid (0.2 g, 89.3%). Mass (m/z): 374.8 [M+H]⁺.

Step 6. N-(3-((1r,4s)-4-aminocyclohexyl)propyl)-6-(2,6-dimethylmorpholino)-2-methylpyridin-3-amine (526)

To a solution of 526-5 (0.2 g, 0.5 mmol) in THF (1 mL) was added BH₃-THF (5 mL) at 25° C. The reaction was stirred at 70° C. for 16 h. The mixture was diluted with EA (200 mL) and washed with water (200 mL×3). The organic phase was concentrated and purification by Prep-TLC (DCM:MeOH=10:1) to give 526 (12.5 mg, 6.5%) as a white solid. ¹H NMR (400 MHZ, CD₃OD) δ 6.96 (d, J=8.6 Hz, 1H), 6.54 (d, J=8.6 Hz, 1H), 3.78-3.67 (m, 4H), 3.02 (t, J=7.2 Hz, 2H), 2.56-2.54 (m, 1H), 2.32-2.22 (m, 5H), 1.80-1.78 (m, 4H), 1.60-1.68 (m, 2H), 1.37-0.91 (m, 13H). Mass (m/z): 360.9 [M+H]⁺.

Compound 527

### 6-(4-methoxypiperidin-1-yl)-2-methyl-3-nitropyridine

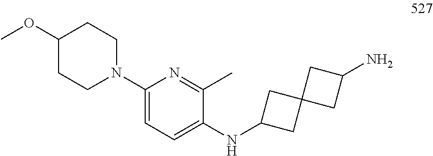 [SMILES: COC1CCN(c2ccc(NC3CC4(CC(N)C4)C3)c(C)n2)CC1]

The title compound 527 (43 mg, 27.8) as a white solid was prepared from tert-butyl (6-((6-(4-methoxypiperidin-1-yl)-2-methylpyridin-3-yl)amino)spiro[3.3]heptan-2-yl)carbamate (200 mg, 0.43 mmol), DCM (20 mL) and TFA (4 mL) according to the procedure for 24. H NMR (400 MHZ, CD₃OD) δ 6.83 (d, J=8.7 Hz, 1H), 6.54 (d, J=8.7 Hz, 1H), 3.68 (d, J=7.8 Hz, 3H), 3.34 (m, 4H), 2.90 (t, 2H), 2.53-2.29 (m, 3H), 2.24 (m, 4H), 1.99-1.91 (m, 2H), 1.91-1.74 (m, 4H), 1.57 (s, 2H), 1.28 (t, J=7.9 Hz, 1H). Mass (m/z): 330.8 [M+H]⁺.

### Compound 528

### N2-(6-(3-oxa-8-azabicyclo[3.2.1]octan-8-yl)-2-methylpyridin-3-yl)spiro[3.3]heptane-2,6-diamine

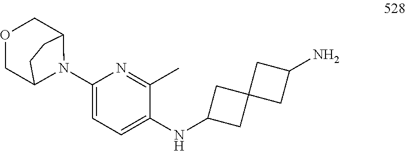 [SMILES: Cc1nc(N2C3CCC2COC3)ccc1NC1CC2(CC(N)C2)C1]

The desired product as a yellow solid (23.2 mg, 16.1% yield) was prepared from tert-butyl (6-((6-(3-oxa-8-azabicyclo[3.2.1]octan-8-yl)-2-methylpyridin-3-yl)amino)spiro[3.3]heptan-2-y 1) carbamate (150 mg, 0.38 mmol), 1,4-dioxane (3 mL) and HCl/1,4-dioxane (1 mL) according to the procedure for 37. ¹H NMR (400 MHZ, DMSO-d₆) δ 6.71 (dd, J=8.7, 4.0 Hz, 1H), 6.49 (d, J=8.6 Hz, 1H), 4.45 (d, J=7.3 Hz, 1H), 4.25 (s, 2H), 3.91-3.74 (m, 1H), 3.63 (m, 3H), 3.47 (d, J=9.8 Hz, 2H), 3.23-3.13 (m, 1H), 2.42-2.22 (m, 3H), 2.18 (s, 3H), 2.15-2.09 (m, 1H), 1.84 (m, 4H), 1.74-1.63 (m, 3H). Mass (m/z): 328.8 [M+H]⁺.

### Compound 529

### N2-(6-(4-(2-methoxypropan-2-yl)piperidin-1-yl)-2-methylpyridin-3-yl)spiro[3.3]-heptane-2,6-diamine

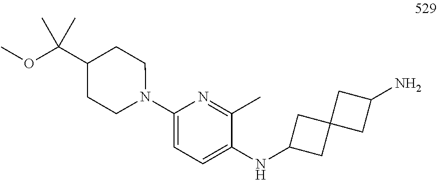 [SMILES: COC(C)(C)C1CCN(c2ccc(NC3CC4(CC(N)C4)C3)c(C)n2)CC1]

The title compound 529 (2.8 mg) as a yellow oil was prepared from tert-butyl (6-((6-(4-(2-methoxypropan-2-yl)piperidin-1-yl)-2-methylpyridin-3-yl)amino)spiro[3.3]heptan-2-yl)carbamate (40 mg, 0.08 mmol), DCM (10 mL) and TFA (1 mL) according to the procedure for 24. ¹H NMR (400 MHZ, CD₃OD) δ 7.94 (d, J=9.4 Hz, 1H), 7.56 (d, J=8.8 Hz, 1H), 7.20 (d, J=9.6 Hz, 1H), 7.15 (dd, J=8.8, 2.4 Hz, 1H), 6.93 (d, J=2.4 Hz, 1H), 4.00 (d, J=13.4 Hz, 2H), 3.52-3.40 (m, 2H), 3.37-3.27 (m, 3H), 1.93 (d, J=13.2 Hz, 2H), 1.72-1.59 (m, 2H), 1.22 (s, 3H). Mass (m/z): 373.3 [M+H]⁺.

### Compound 530

### 2-N-(2-methyl-6-{2-oxa-7-azaspiro[3.5]nonan-7-yl}pyridin-3-yl)spiro[3.3]heptane-2,6-diamine

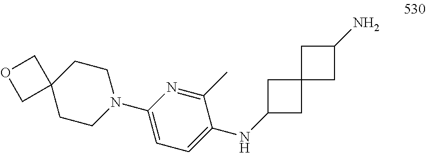 [SMILES: Cc1nc(N2CCC3(CC2)COC3)ccc1NC1CC2(CC(N)C2)C1]

The title compound 530 (6.5 mg, 4.2%) as a light yellow solid was prepared from tert-butyl {6-[(2-methyl-6-{2-oxa-7-azaspiro[3.5]nonan-7-yl}pyridin-3-yl)amino]spiro [3.3]heptan-2-yl}a mino formate (70 mg, 0.16 mmol), DCM (5 mL) and TFA (1 mL) according to the procedure for 24. ¹H NMR (400 MHZ, DMSO-d₆) δ 8.30 (s, 1H), 7.44 (d, J=8.6 Hz, 1H), 6.93 (d, J=8.7 Hz, 1H), 5.83 (d, J=6.0 Hz, 1H), 5.24 (d, J=66.0 Hz, 1H), 4.12 (s, 2H), 3.87-3.72 (m, 4H), 3.67 (s, 1H), 3.50 (d, J=7.6 Hz, 2H), 3.34 (s, 6H), 2.40 (d, J=4.3 Hz, 1H), 2.33 (s, 2H), 2.19 (d, J=3.9 Hz, 2H), 2.12-1.93 (m, 3H), 1.83 (s, 1H), 1.23 (s, 1H). Mass (m/z): 343.1 [M+H]⁺.

### Compound 531

### N2-(2-(4-isopropylpiperidin-1-yl)pyrimidin-5-yl)-N6-(2,2, 2-trifluoroethyl)spiro[3.3]heptane-2, 6-diamine

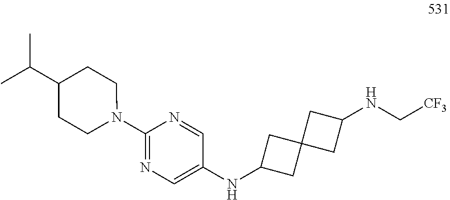 [SMILES: CC(C)C1CCN(c2ncc(NC3CC4(CC(NCC(F)(F)F)C4)C3)cn2)CC1]

The desired product (3.3 mg, 2.9% yield) as a yellow solid was prepared from DIEA (106 mg, 0.82 mmol), 2,2,2-trifluoroethyl mg, 0.55 triflate (127 mmol), 2-N-[2-(4-isopropylpiperidin-1-yl)pyrimidin-5-yl]spiro [3.3]heptane-2,6-diamine hydrochloride (100 mg, 0.27 mmol) and dioxane (20 mL) according to the procedure for 414. ¹H NMR (400 MHz, CD₃OD) δ 7.81 (d, J=0.7 Hz, 2H), 4.49-4.44 (m, 2H), 3.66 (t, J=7.5 Hz, 1H), 3.21 (d, J=7.5 Hz, 1H), 3.09 (d, J=9.9 Hz, 2H), 2.74-2.67 (m, 2H), 2.48 (ddd, J=11.3, 6.3, 2.8 Hz, 1H), 2.41-2.29 (m, 2H), 2.22-2.16 (m, 1H), 1.87-1.78 (m, 4H), 1.73-1.68 (m, 2H), 1.45-1.38 (m, 1H), 1.19 (m, 3H), 0.88 (d, J=6.8 Hz, 6H). Mass (m/z): 412.3 [M+H]⁺.

### Compound 532

### N-(((1r,4r)-4-aminocyclohexyl)methyl)-4-(4-ethyl-3,5-dimethylpiperazin-1-yl)aniline

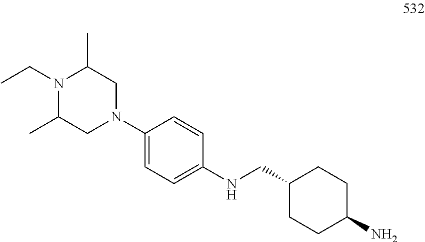 [SMILES: CCN1C(C)CN(c2ccc(NC[C@H]3CC[C@H](N)CC3)cc2)CC1C]

The title compound 532 (20 mg, 20%) as a yellow solid (21.1 mg, 18.5%) was prepared from tert-butyl ((1r,4r)-4-(((4-(4-ethyl-3,5-dimethylpiperazin-1-yl)phenyl)amino)methyl)cyclohexyl)carbamat e (0.12 g, 0.3 mmol), DCM (5 mL) and TFA (1 mL) according to the procedure for 24. ¹H NMR (400 MHZ, CD₃OD) δ 6.83 (d, J=8.8 Hz, 2H), 6.59 (d, J=8.8 Hz, 2H), 3.24 (d, J=11.0 Hz, 2H), 2.98 (q, J=7.2 Hz, 2H), 2.84-2.83 (m, 4H), 2.78-2.66 (m, 1H), 2.38 (t, J=11.0 Hz, 2H), 1.8 9-1.91 (m, 4H), 1.51-1.49 (m, 1H), 1.25-0.91 (m, 13H). Mass (m/z): 345.1 [M+H]⁺.

### Compound 533

### N2-(2-methyl-6-(2-oxa-9-azaspiro [5.5]undecan-9-yl)pyridin-3-yl)spiro[3.3]heptane-2,6-diamine

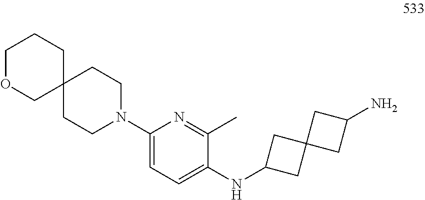 [SMILES: Cc1nc(N2CCC3(CCCOC3)CC2)ccc1NC1CC2(CC(N)C2)C1]

The title compound 533 (51.9 mg, 58.3% yield) as a yellow solid was prepared from tert-butyl (6-((2-methyl-6-(2-oxa-9-azaspiro [5.5]undecan-9-yl)pyridin-3-yl)amino)spiro[3.3]heptan-2-yl) carbamate (200 mg, 0.424 mmol), DCM (10 mL) and TFA (1 mL) according to the procedure for 24. ¹H NMR (400 MHZ, CD₃OD) δ 6.88 (d, J=8.7 Hz, 1H), 6.58 (d, J=8.7 Hz, 1H), 3.78-3.70 (m, 1H), 3.67 (dd, J=10.5, 5.7 Hz, 2H), 3.49 (s, 2H), 3.30 (d, J=1.1 Hz, 1H), 3.29-3.20 (m, 4H), 2.56-2.35 (m, 3H), 2.29 (s, 3H), 2.25 (m, 1H), 1.87-1.75 (m, 4H), 1.68-1.57 (m, 8H). Mass (m/z): 371.27 [M+H]⁺.

### Compound 534

### N-(4-(3-aminopropyl)cyclohexyl)-6-(2,6-dimethylmorpholino)-2-methylpyridin-3-amine

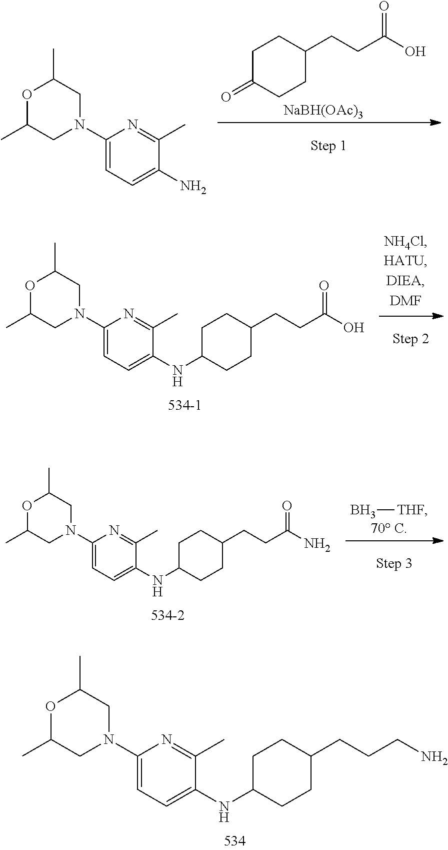

Step 1. Preparation of 3-(4-((6-(2,6-dimethylmorpholino)-2-methylpyridin-3-yl)amino)cyclohexyl) propanoic acid (534-1)

A mixture of 6-(2,6-dimethylmorpholino)-2-methylpyridin-3-amine (500 mg, 2.26 mmol), 3-(4-oxocyclohexyl) propanoic acid (384 mg, 2.26 mmol), and NaBH (OAc); (479 mg, 2.26 mmol) in DCE (10 mL) was stirred overnight at 25° C. After the reaction was completed, solvent was removed under vacuum and the crude was purified through silica gel chromatography to give of 3-(4-((6-(2,6-dimethylmorpholino)-2-methylpyridin-3-yl)amino)cyclohexyl) propanoic acid (300 mg, 35.3%) as a yellow solid. Mass (m/z): 376.1 [M+H]⁺.

Step 2. Preparation of 3-(4-((6-(2,6-dimethylmorpholino)-2-methylpyridin-3-yl)amino) cyclohexyl) propanamide (534-2)

To a solution of 3-(4-((6-(2,6-dimethylmorpholino)-2-methylpyridin-3-yl)amino)cyclohexyl) propanoic acid (300 mg, 1.1 mmol), NH₄Cl (70 mg, 1.3 mmol), HATU (621 mg, 1.6 mmol) and DIEA (281 mg, 2.2 mmol) in DMF (10 ml). The reaction was stirred at R.T. for 18 hours. The solids were filtered and solvent was removed under vacuum. The residue was purified by silica gel column to provide 200 mg of 3-(4-((6-(2,6-dimethylmorpholino)-2-methylpyridin-3-yl)amino) cyclohexyl) propanamide as a yellow solid. Mass (m/z): 375.1 [M+H]⁺.

Step 3. Preparation of N-(4-(3-aminopropyl)cyclohexyl)-6-(2,6-dimethylmorpholino)-2-methylpyridin-3-amine (534) To a solution of 3-(4-((6-(2,6-dimethylmorpholino)-2-methylpyridin-3-yl)amino)cyclohexyl) propanamide (200 mg, 0.53 mmol), BH₃-THF (10 ml) in THF (5 mL). The reaction was stirred at 70° C. for 18 hours. The solids were filtered and solvent was removed under vacuum. The residue was purified by prep-HPLC (column-Xbridge-C_{18 150×19} mm, 5 μm; Mobile phase: ACN-H₂O (0.1% FA), 25%-40%) to afford 534 (8.0 mg) as a white solid. ¹H NMR (400 MHZ, DMSO-d₆) δ 6.86 (d, J=8.8 Hz, 1H), 6.50-6.46 (m, 1H), 3.83-3.78 (m, 3H), 3.61-3.54 (m, 2H), 2.85 (d, J=5.8 Hz, 1H), 2.61 (dd, J=3.8, 1.8 Hz, 1H), 2.51-2.48 (m, 1H), 2.20 (s, 3H), 2.17-2.13 (m, 1H), 2.12 (d, J=1.6 Hz, 1H), 2.09 (d, J=3.2 Hz, OH), 1.56-1.28 (m, 10H), 1.25-1.18 (m, 3H), 1.10 (d, J=6.2 Hz, 6H). Mass (m/z): 361.3 [M+H]⁺.

### Compound 535

### N2-(4-(3,5-dimethyl-4-(2,2,2-trifluoroethyl) piperazin-1-yl)-3-fluorophenyl) spiro-[3.3]heptane-2.6-diamine

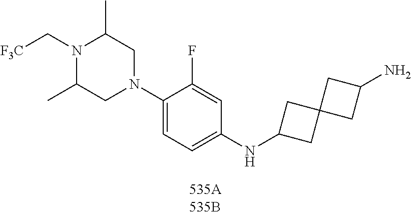 [SMILES: CC1CN(c2ccc(NC3CC4(CC(N)C4)C3)cc2F)CC(C)N1CC(F)(F)F]

The title compound 535A (5.4 mg) as a white solid and compound 535B (5.8 mg) as a white solid were prepared from tert-butyl (6-((4-(3,5-dimethyl-4-(2,2,2-trifluoroethyl) piperazin-1-yl)-3-fluoro-phenyl)amino)spiro[3.3]h eptan-2-yl)carbamate (150 mg, 0.29 mmol), DCM (10 mL) and TFA (1 mL) according to the procedure for 24. 535A: HNMR (400 MHZ, DMSO-d₆) δ 6.81 (t, J=8.8 Hz, 1H), 6.31-6.26 (m, 2H), 3.76-3.59 (m, 2H), 3.34 (q, J=9.8 Hz, 2H), 3.03 (dd, J=8.6, 2.2 Hz, 2H), 2.93 (d, J=6.2 Hz, 2H), 2.54-2.30 (m, 6H), 2.20-2.09 (m, 2H), 1.94-1.86 (m, 2H), 1.11 (d, J=6.2 Hz, 6H). Mass (m/z): 415.3 [M+H]⁺. HPLC: Rt: 3.919 min (Column: XBRIDGE 2.1*50 mm, 3.5 μm; Mobile Phase: H₂O (0.05% TFA) ACN (0.05% TFA)/ACN from 0% to 60% over 7 minutes, 7-8 min, ACN from 60% to 100%; 0.8 mL/min). 535B: ¹H NMR (400 MHZ, DMSO-d₆) δ 6.82 (d, J=8.6 Hz, 1H), 6.32-6.21 (m, 2H), 3.68 (d, J=32.8 Hz, 2H), 3.34 (d, J=10.0 Hz, 2H), 3.09-3.00 (m, 2H), 2.94 (dd, J=14.2, 13.6 Hz, 2H), 2.61-2.48 (m, 2H), 2.40 (t, J=10.8 Hz, 4H), 2.14 (s, 2H), 1.96-1.78 (m, 2H), 1.11 (d, J=6.2 Hz, 6H). HPLC: Rt: 3.921 min (Column: XBRIDGE 2.1*50 mm, 3.5 μm; Mobile Phase: H₂O (0.05% TFA)/ACN (0.05% TFA), ACN from 0% to 60% over 7 minutes, 7-8 min, ACN from 60% to 100%; 0.8 mL/min).

### Compound 536

N2-(6-(4-methoxy-4-methylpiperidin-1-yl)-2-methylpyridin-3-yl)spiro[3.3]heptane-2,6-diamine

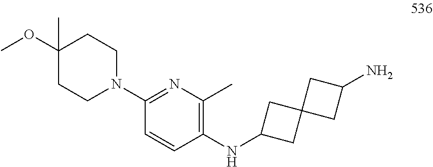 [SMILES: COC1(C)CCN(c2ccc(NC3CC4(CC(N)C4)C3)c(C)n2)CC1]

The title compound 536 (25.0 mg) was prepared in a total yield of 47.3% as a yellow solid from tert-butyl (6-((6-(4-methoxy-4-methylpiperidin-1-yl)-2-methylpyridin-3-yl)amino)spiro[3.3]heptan-2-yl) carbamate (68 mg, 0.153 mmol), TFA (1 mL) and DCM (10 mL) according to the procedure for 24. ¹H NMR (400 MHZ, DMSO-d₆) δ 8.41 (s, 3H), 7.20-6.71 (m, 2H), 4.86 (s, 1H), 3.75-3.42 (m, 5H), 3.12 (s, 5H), 2.43-2.15 (m, 8H), 1.97 (s, 2H), 1.73 (d, J=13.6 Hz, 2H), 1.53 (s, 2H), 1.12 (s, 3H). Mass (m/z): 345.3 [M+H]⁺

### Compound 537

### N2-(6-(4-((2S,6R)-2,6-dimethylmorpholino) piperidin-1-yl)-2-methylpyridin-3-yl)spiro[3.3]heptane-2,6-diamine

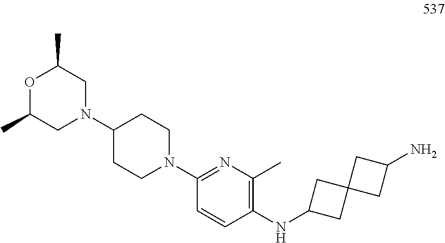 [SMILES: Cc1nc(N2CCC(N3C[C@@H](C)O[C@@H](C)C3)CC2)ccc1NC1CC2(CC(N)C2)C1]

The title compound 537 (17.6 mg) was prepared in a total yield of 43.7% as a yellow solid from tert-butyl (6-((6-(4-((2S,6R)-2,6-dimethylmorpholino) piperidin-1-yl)-2-methylpyridin-3-yl)amino)spiro[3.3]heptan-2-yl)carbamate (50 mg, 0.097 mmol), TFA (1 mL), and DCM (10 mL) according to the procedure for 24. ¹H NMR (400 MHZ, DMSO-d₆) δ 8.74-8.35 (m, 5H), 6.73 (d, J=8.8 Hz, 1H), 6.56 (d, J=8.8 Hz, 1H), 4.59 (s, 1H), 4.27-3.94 (m, 4H), 3.68-3.46 (m, 3H), 3.19-3.07 (m, 3H), 2.56 (d, J=12.0 Hz, 2H), 2.45 (ddt, J=9.2, 5.2, 1.6 Hz, 4H), 2.39-2.31 (m, 2H), 2.28-2.10 (m, 8H), 1.91 (td, J=11.2, 10.4, 5.2 Hz, 2H), 1.70 (s, 2H), 1.12 (d, J=6.4 Hz, 6H). Mass (m/z): 434.3 [M+H]⁺.

### Compound 538

### N-((2r,5s)-5-(aminomethyl) adamantan-2-yl)-2-methyl-6-morpholinopyridin-3-amine

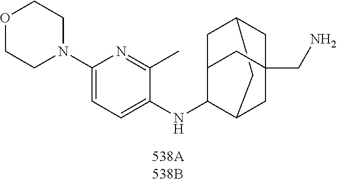 [SMILES: Cc1nc(N2CCOCC2)ccc1NC1C2CC3CC1CC(CN)(C3)C2]

The title compound 538A (Rt-9.41 min; 7.8 mg) was prepared in a two-step overall yield of 6.5% as a white powder and compound 538B (Rt=10.48 min; 3.1 mg) was prepared in a two-step overall yield of 2.5% as a white powder from 2-methyl-6-morpholinopyridin-3-amine (200 mg, 1.03 mmol) and 4-oxoadamantane-1-carboxylic acid (301 mg, 1.55 mmol) according to the procedure for 84, which were purified by Prep-HPLC (Column: X Select-CSH-Prep 5 μm OBD, 19*150 mm; ACN/water (0.5% TFA)=0%-0%-30%-95%-95%-0%, 0 min-2 min-12 min-12.5 min-13.5 min-15.0 min). 538A: ¹H NMR (400 MHZ, DMSO-d₆) δ 6.86 (d, J=8.8 Hz, 1H), 6.52 (d, J=8.7 Hz, 1H), 3.86 (d, J=6.3 Hz, 1H), 3.68 (t, J=4.8 Hz, 4H), 3.18 (t, J=4.8 Hz, 4H), 2.28 (s, 3H), 2.22 (s, 2H), 1.96 (d, J=13.4 Hz, 4H), 1.87 (s, 1H), 1.51 (s, 4H), 1.42 (d, J=3.1 Hz, 2H), 1.35 (d, J=12.1 Hz, 2H). Mass (m/z): 357.3 [M+H]⁺. 538B: ¹H NMR (400 MHz, DMSO-d₆) δ 6.91 (dd, J=8.8, 3.6 Hz, 1H), 6.53 (d, J=8.7 Hz, 1H), 3.76 (d, J=6.9 Hz, 1H), 3.72-3.63 (m, 4H), 3.19 (t, J=4.8 Hz, 4H), 2.27 (d, J=2.3 Hz, 3H), 2.20 (s, 2H), 1.95 (d, J=21.2 Hz, 3H), 1.72 (q, J=14.8, 13.7 Hz, 6H), 1.48-1.37 (m, 2H), 1.28-1.13 (m, 3H). Mass (m/z): 357.3 [M+H]⁺.

### Compound 539

### N-((2r,5s)-5-(aminomethyl) adamantan-2-yl)-6-(2,6-dimethylmorpholino)-5-fluoro-2-methylpyridin-3-amine

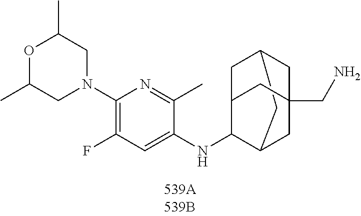 [SMILES: Cc1nc(N2CC(C)OC(C)C2)c(F)cc1NC1C2CC3CC1CC(CN)(C3)C2]

The title compound 539A (Rt-6.04 min; 2.1 mg) was prepared in a two-step overall yield of 2.7% as a white powder and compound 539B (Rt-6.82 min; 1.1 mg) was prepared in a two-step overall yield as of 1.4% a white powder from 6-(2,6-dimethylmorpholino)-5-fluoro-2-methylpyridin-3-amine (70 mg, 0.29 mmol) and 4-oxoadamantane-1-carboxylic acid (85 mg, 0.44 mmol) according to the procedure for 84. The target products were purified by Prep-HPLC (Column: X Select-CSH-Prep 5 μm OBD, 19*150 mm; ACN/water (0.5% TFA)=15%-45%-95%-95%-10%, 0 min-7 min-7.5 min-8.5 min-10.0 min). 539A: ¹H NMR (400 MHZ, DMSO-d₆) δ 6.79 (d, J=14.6 Hz, 1H), 4.21 (d, J=6.0 Hz, 1H), 3.74-3.63 (m, 2H), 2.36 (d, J=2.0 Hz, 2H), 2.29-2.24 (m, 5H), 2.02-1.85 (m, 5H), 1.55 (q, J=12.2 Hz, 4H), 1.45 (d, J=2.8 Hz, 2H), 1.38 (d, J=12.2 Hz, 2H), 1.11 (d, J=6.2 Hz, 6H). Mass (m/z): 403.5 [M+H]⁺. 539B: ¹H NMR (400 MHZ, DMSO-d₆) δ 6.82 (dd, J=14.6, 6.4 Hz, 1H), 6.66 (s, 1H), 4.06 (d, J=6.4 Hz, 1H), 3.73-3.61 (m, 2H), 3.37 (s, 3H), 2.38-2.33 (m, 2H), 2.25 (d, J=1.1 Hz, 3H), 2.18 (s, 1H), 2.02-1.94 (m, 5H), 1.91 (s, 1H), 1.78 (d, J=13.2 Hz, 2H), 1.71-1.61 (m, 4H), 1.44 (t, J=6.8 Hz, 2H), 1.39 (s, 2H), 1.17 (s, 1H), 1.09 (d, J=6.3 Hz, 6H), 0.86-0.80 (m, 2H). Mass (m/z): 403.5 [M+H]⁺.

### Compound 540

### N4-(6-((3R,5S)-3,5-dimethyl-4-(2,2,2-trifluoroethyl) piperazin-1-yl)-2-methylpyridin-3-yl)adamantane-1,4-diamine

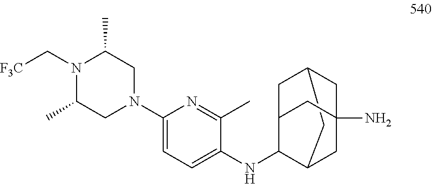 [SMILES: Cc1nc(N2C[C@@H](C)N(CC(F)(F)F)[C@@H](C)C2)ccc1NC1C2CC3CC1CC(N)(C3)C2]

The title compound 540 (33.0 mg) was prepared in a yield of 52.6% as a white powder from 6-((3R,5S)-3,5-dimethyl-4-(2,2,2-trifluoroethyl) piperazin-1-yl)-2-methylpyridin-3-amine (42 mg, 0.14 mmol) and tert-butyl (4-oxoadamantan-1-yl)carbamate (44 mg, 0.17 mmol) according to the procedure for 20. ¹H NMR (400 MHZ, DMSO-d₆) δ 8.08 (d, J=34.9 Hz, 3H), 6.98 (s, 1H), 6.62 (s, 1H), 3.86 (d, J=12.1 Hz, 2H), 3.39 (d, J=10.8 Hz, 3H), 2.76 (s, 2H), 2.36 (s, 4H), 2.12 (d, J=22.3 Hz, 3H), 2.01 (d, J=22.0 Hz, 2H), 1.96-1.84 (m, 3H), 1.79 (d, J=16.9 Hz, 3H), 1.68 (d, J=12.7 Hz, 1H), 1.55 (d, J=11.3 Hz, 1H), 1.36 (d, J=12.5 Hz, 1H), 1.10 (d, J=6.2 Hz, 6H). Mass (m/z): 451.7 [M+H]⁺.

### Compound 541

### N1-(6-((3R,5S)-3,5-dimethyl-4-(2,2,2-trifluoroethyl) piperazin-1-yl)-2-methylpyridin-3-yl)cyclopentane-1,3-diamine

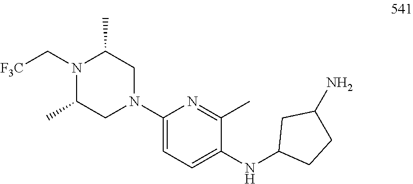 [SMILES: Cc1nc(N2C[C@@H](C)N(CC(F)(F)F)[C@@H](C)C2)ccc1NC1CCC(N)C1]

The title compound 541 (31.4 mg) was prepared in a yield of 57.89% as a white powder from 6-((3R,5S)-3,5-dimethyl-4-(2,2,2-trifluoroethyl) piperazin-1-yl)-2-methylpyridin-3-amine (42 mg, 0.14 mmol) and tert-butyl (3-oxocyclopentyl)carbamate (42 mg, 0.21 mmol) according to the procedure for 20. ¹H NMR (400 MHZ, DMSO-d₆) δ 8.20 (d, J=29.6 Hz, 3H), 7.01 (s, 1H), 6.67 (s, 1H), 4.47 (s, 1H), 3.88 (s, 3H), 3.64 (t, J=53.5 Hz, 4H), 2.76 (s, 2H), 2.43-2.23 (m, 4H), 2.10 (d, J=15.5 Hz, 1H), 2.02-1.71 (m, 3H), 1.69-1.45 (m, 1H), 1.10 (d, J=6.2 Hz, 6H). Mass (m/z): 386.3 [M+H]⁺.

### Compound 542

### N1-(6-(3R,5S)-3,5-dimethyl-4-(2,2,2-trifluoroethyl) piperazin-1-yl)-2-methylpyridin-3-yl)cyclohexane-1,4-diamine

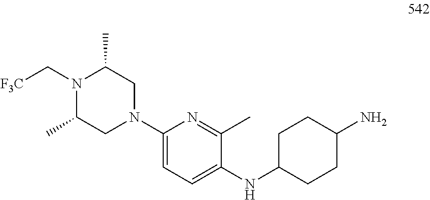 [SMILES: Cc1nc(N2C[C@@H](C)N(CC(F)(F)F)[C@@H](C)C2)ccc1NC1CCC(N)CC1]

The title compound 542 (38.4 mg) was prepared in a yield of 69.19% as a white powder from 6-((3R,5S)-3,5-dimethyl-4-(2,2,2-trifluoroethyl) piperazin-1-yl)-2-methylpyridin-3-amine (44 mg, 0.21 mmol) and tert-butyl (4-oxocyclohexyl)carbamate (42 mg, 0.21 mmol) according to the procedure for 20. ¹H NMR (400 MHZ, DMSO-d₆) δ 8.41-8.21 (m, 3H), 7.04 (s, 1H), 6.65 (s, 1H), 3.88 (d, J=12.9 Hz, 2H), 3.43 (d, J=24.3 Hz, 1H), 3.11 (s, 1H), 2.92 (s, 1H), 2.75 (s, 2H), 2.35 (s, 5H), 1.99 (s, 2H), 1.75 (d, J=26.4 Hz, 3H), 1.60 (s, 1H), 1.44 (d, J=11.9 Hz, 1H), 1.09 (d, J=6.2 Hz, 6H). Mass (m/z): 400.4 [M+H]⁺.

### Compound 543

### N-(((1r,4R)-4-aminocyclohexyl)methyl)-6-((3R,5S)-3,5-dimethyl-4-(2,2,2-trifluoroethyl) piperazin-1-yl)-2-methylpyridin-3-amine

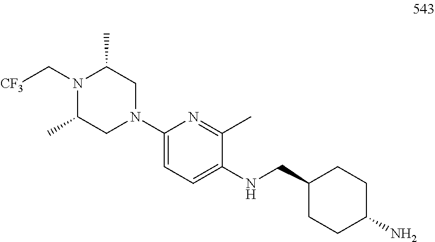 [SMILES: Cc1nc(N2C[C@@H](C)N(CC(F)(F)F)[C@@H](C)C2)ccc1NC[C@H]1CC[C@H](N)CC1]

The title compound 543 (32.3 mg) was prepared in a yield of 56.2% as a white powder from 6-((3R,5S)-3,5-dimethyl-4-(2,2,2-trifluoroethyl) piperazin-1-yl)-2-methylpyridin-3-amine (44 mg, 0.21 mmol) and tert-butyl ((1r,4r)-4-formylcyclohexyl)carbamate (47 mg, 0.21 mmol) according to the procedure for 24. ¹H NMR (400 MHZ, DMSO-d₆) δ 8.03 (s, 3H), 6.94 (s, 1H), 6.63 (s, 1H), 4.57 (s, 1H), 3.82 (s, 2H), 2.99-2.69 (m, 6H), 2.42-2.19 (m, 5H), 1.89 (dd, J=27.9, 15.3 Hz, 4H), 1.49 (s, 1H), 1.35-1.21 (m, 2H), 1.08 (d, J=6.0 Hz, 6H), 0.98 (d, J=12.9 Hz, 2H). Mass (m/z): 414.7 [M+H]⁺.

### Compound 544

### N-(((1r,4R)-4-aminocyclohexyl)methyl)-6-((2S,6R)-2,6-dimethylmorpholino)-2-ethylpyridin-3-amine

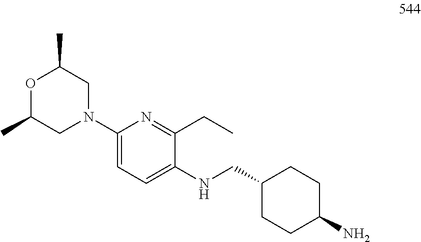 [SMILES: CCc1nc(N2C[C@@H](C)O[C@@H](C)C2)ccc1NC[C@H]1CC[C@H](N)CC1]

The title compound 544 (30.9 mg) was prepared in a total yield of 28.3% as a purple solid according to the procedure for compound 24. ¹H NMR (400 MHZ, DMSO-d₆) δ 8.10 (s, 3H), 6.88 (br, 1H), 6.53 (br, 1H), 4.66-4.29 (m, 1H), 3.98-3.72 (m, 2H), 3.71-3.55 (m, 2H), 2.96-2.82 (m, 2H), 2.26-2.10 (m, 2H), 2.03-1.91 (m, 2H), 1.89-1.80 (m, 2H), 1.57-1.43 (m, 1H), 1.35-1.25 (m, 2H), 1.18-1.10 (m, 9H), 1.05-0.92 (m, 2H). Mass (m/z): 347.2 [M+H]⁺.

### Compound 545

### N²-(6-((2S,6R)-2,6-dimethylmorpholino)-2-ethylpyridin-3-yl)spiro[3.3]heptane-2,6-diamine

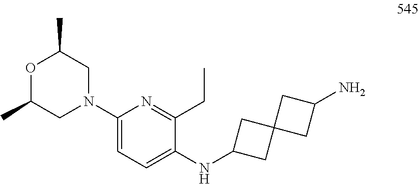 [SMILES: CCc1nc(N2C[C@@H](C)O[C@@H](C)C2)ccc1NC1CC2(CC(N)C2)C1]

The title compound 545 (16.4 mg) was prepared in a total yield of 31.8% as a yellow solid according to the procedure for compound 24. ¹H NMR (400 MHZ, DMSO-d₆) δ 8.14 (d, J=5.2 Hz, 3H), 6.76 (br, 1H), 6.51 (br, 1H), 4.72-4.43 (m, 1H), 3.94-3.78 (m, 2H), 3.70-3.48 (m, 5H), 2.62-2.53 (m, 2H), 2.43-2.33 (m, 2H), 2.22-2.13 (m, 4H), 1.97-1.85 (m, 2H), 1.17-1.12 (m, 9H). Mass (m/z): 345.2 [M+H]⁺.

### Compound 546

### N2-(6-((2R,6S)-2-ethyl-6-methylmorpholino)-2-methylpyridin-3-yl)spiro[3.3]heptane-2,6-diamine

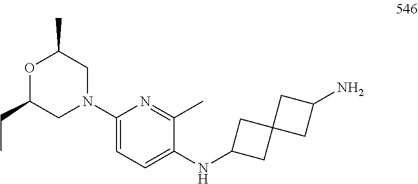 [SMILES: CC[C@@H]1CN(c2ccc(NC3CC4(CC(N)C4)C3)c(C)n2)C[C@H](C)O1]

The title compound 546 (12.3 mg) was prepared in a total yield of 52.8% as a light yellow solid according to the procedure for compound 24. ¹H NMR (400 MHZ, DMSO-d₆) δ 7.98 (s, 3H), 6.80-6.66 (m, 1H), 6.56-6.46 (m, 1H), 4.61-4.49 (m, 1H), 4.41-4.31 (m, 1H), 3.88-3.78 (m, 2H), 3.67-3.52 (m, 4H), 2.29-2.07 (m, 8H), 2.01-1.83 (m, 2H), 1.53-1.43 (m, 2H), 1.14 (d, J=6.1 Hz, 3H), 0.94 (d, J=7.4 Hz, 3H). Mass (m/z): 345.3 [M+H]⁺.

### Compound 547

### N²-(6-((2R,6R)-2-ethyl-6-methylmorpholino)-2-methylpyridin-3-yl)spiro[3.3]heptane-2,6-diamine

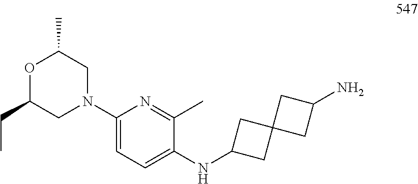 [SMILES: CC[C@@H]1CN(c2ccc(NC3CC4(CC(N)C4)C3)c(C)n2)C[C@@H](C)O1]

The title compound 547 (13.9 mg) was prepared in a total yield of 63.5% as a light yellow solid according to the procedure for compound 24. ¹H NMR (400 MHZ, DMSO-d₆) δ 7.86 (s, 3H), 6.72 (d, J=8.7 Hz, 1H), 6.48 (d, J=8.7 Hz, 1H), 3.97-3.87 (m, 1H), 3.72-3.67 (m, 1H), 3.66-3.60 (m, 1H), 3.58-3.52 (m, 1H), 3.40-3.37 (m, 1H), 3.21 (dd, J=12.2, 3.5 Hz, 1H), 3.05 (dd, J=12.2, 5.5 Hz, 1H), 2.82 (dd, J=12.1, 6.6 Hz, 1H), 2.45-2.34 (m, 2H), 2.26-2.04 (m, 7H), 1.94-1.85 (m, 2H), 1.69-1.59 (m, 2H), 1.53-1.43 (m, 1H), 1.16 (d, J=6.4 Hz, 3H), 0.88 (t, J=7.4 Hz, 3H). Mass (m/z): 345.3 [M+H]⁺.

### Compound 548

### N-(((1r,4R)-4-aminocyclohexyl)methyl)-6-((2R,6R)-2,6-diethylmorpholino)-2-methylpyridin-3-amine

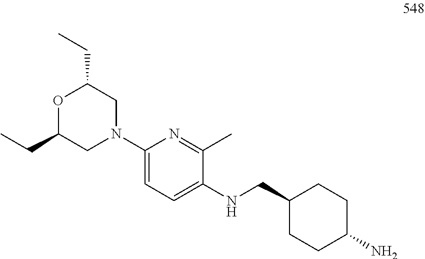 [SMILES: CC[C@@H]1CN(c2ccc(NC[C@H]3CC[C@H](N)CC3)c(C)n2)C[C@@H](CC)O1]

The title compound 548 (13.6 mg) was prepared in a total yield of 37.8% as a light yellow solid according to the procedure for compound 24. ¹H NMR (400 MHZ, DMSO-d₆) δ 7.92 (s, 3H), 6.82 (d, J=8.8 Hz, 1H), 6.50 (d, J=8.7 Hz, 1H), 4.48-4.37 (m, 1H), 3.67-3.61 (m, 1H), 3.28 (dd, J=12.0, 3.3 Hz, 2H), 2.96 (dd, J=12.2, 6.0 Hz, 2H), 2.87-2.79 (m, 2H), 2.21 (s, 3H), 1.98-1.92 (m, 2H), 1.89-1.81 (m, 2H), 1.68-1.60 (m, 2H), 1.51-1.44 (m, 2H), 1.28-1.22 (m, 2H), 1.04-0.96 (m, 2H), 0.90 (t, J=7.4 Hz, 6H). Mass (m/z): 361.3 [M+H]⁺.

### Compound 549

### N²-(6-(2S, 6R)-2,6-diethylmorpholino)-2-methylpyridin-3-yl)spiro[3.3]heptane-2,6-diamine

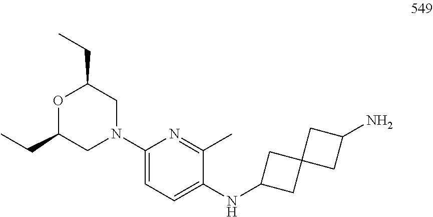 [SMILES: CC[C@@H]1CN(c2ccc(NC3CC4(CC(N)C4)C3)c(C)n2)C[C@H](CC)O1]

The title compound 549 (13.6 mg) was prepared in a total yield of 66.3% as a light yellow solid according to the procedure for compound 24. ¹H NMR (400 MHZ, Methanol-d₄) δ 6.90 (d, J=8.8 Hz, 1H), 6.56 (d, J=8.5 Hz, 1H), 3.81-3.75 (m, 2H), 3.51-3.43 (m, 2H), 2.65-2.45 (m, 4H), 2.43-2.25 (m, 7H), 2.21-2.14 (m, 2H), 2.05-1.97 (m, 2H), 1.62-1.52 (m, 4H), 1.02 (t, J=7.4 Hz, 6H). Mass (m/z): 359.2 [M+H]⁺.

### Compound 550

### N2-(6-(2R,6R)-2,6-diethylmorpholino)-2-methylpyridin-3-yl)spiro[3.3]heptane-2,6-diamine

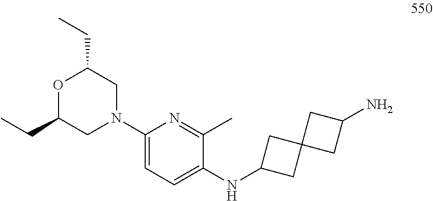 [SMILES: CC[C@@H]1CN(c2ccc(NC3CC4(CC(N)C4)C3)c(C)n2)C[C@@H](CC)O1]

The title compound 550 (13.8 mg) was prepared in a total yield of 38.5% as a light yellow solid according to the procedure for compound 24. ¹H NMR (400 MHZ, Methanol-d₄) δ 6.92 (d, J=6.8 Hz, 1H), 6.55 (d, J=14.7 Hz, 1H), 3.83-3.72 (m, 2H), 3.41-3.33 (m, 2H), 3.07-2.96 (m, 2H), 2.68-2.52 (m, 2H), 2.50-2.13 (m, 9H), 2.05-1.95 (m, 2H), 1.83-1.69 (m, 2H), 1.58-1.48 (m, 2H), 0.98 (t, J=7.5 Hz, 6H). Mass (m/z): 359.2 [M+H]⁺.

### Compound 551

### N1-(6-((2R,6S)-2-ethyl-6-methylmorpholino)-2-methylpyridin-3-yl)cyclohexane-1,4-diamine

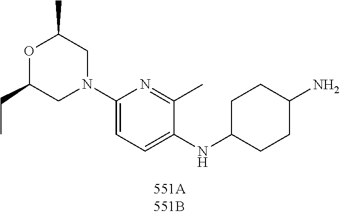 [SMILES: CC[C@@H]1CN(c2ccc(NC3CCC(N)CC3)c(C)n2)C[C@H](C)O1]

The title compound 551A and 551B were prepared according to the procedure for compound 24. The crude residue was purified by preparative TLC (H₂O: MeOH: Dichloromethane=0.1:1:5) to afford compound 551A in 26.1% yield as a light yellow solid and 551B in 37.0% yield as a light yellow solid. 551A: ¹H NMR (400 MHZ, Methanol-d₄) δ 7.04 (d, J=8.7 Hz, 1H), 6.57 (d, J=8.8 Hz, 1H), 3.83-3.78 (m, 2H), 3.73-3.69 (m, 2H), 3.57-3.53 (m, 2H), 2.34 (s, 3H), 2.31-2.27 (m, 2H), 2.06-1.98 (m, 4H), 1.81-1.75 (m, 4H), 1.61-1.58 (m, 2H), 1.22 (d, J=6.2 Hz, 3H), 1.01 (t, J=7.4 Hz, 3H). Mass (m/z): 333.3 [M+H]⁺. HPLC: Rt=2.846 mins (Agilent, poroshell 120, SB—C18 2.7 μm, 4.6×50 mm, ACN/Water (0.1% FA)=5%-5%-95%-95%-95%-5%, 0 min-0.5 min-10 min-10.5 min-12.0 min) 551B: ¹H NMR (400 MHz, Methanol-d₄) δ 7.06 (d, J=10.3 Hz, 1H), 6.59 (d, J=9.3 Hz, 2H), 3.86-3.67 (m, 3H), 3.64-3.56 (m, 1H), 3.53-3.48 (m, 1H), 3.24-3.07 (m, 3H), 2.29 (s, 3H), 2.17-2.01 (m, 4H), 1.62-1.49 (m, 4H), 1.37-1.31 (m, 2H), 1.22 (d, J=3.6 Hz, 3H), 1.07-0.95 (m, 3H). Mass (m/z): 333.3 [M+H]⁺. HPLC: Rt=2.607 mins (Agilent, poroshell 120, SB—C18 2.7 μm, 4.6×50 mm, ACN/Water (0.1% FA)=5%-5%-95%-95%-95%-5%, 0 min-0.5 min-10 min-10.5 min-12.0 min).

### Compound 552

### N1-(6-((2R,6R)-2,6-diethylmorpholino)-2-methylpyridin-3-yl)cyclohexane-1,4-diamine

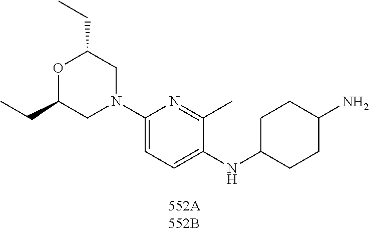 [SMILES: CC[C@@H]1CN(c2ccc(NC3CCC(N)CC3)c(C)n2)C[C@@H](CC)O1]

The title compound 552A, 552B was prepared according to the procedure for compound 24. The crude residue was purified by preparative TLC (H₂O: MeOH: Dichloromethane=0.1:1:5) to afford compound 552A in a 14.4% yield as a light yellow solid and 552B in a 23.3% yield as a light yellow solid. 552A: ¹H NMR (400 MHZ, Methanol-d₄) δ 7.04 (d, J=8.7 Hz, 1H), 6.57 (d, J=8.6 Hz, 1H), 4.22-4.11 (m, 1H), 3.84-3.77 (m, 2H), 3.57-3.52 (m, 1H), 3.51-3.44 (m, 2H), 3.27-3.19 (m, 2H), 2.34 (s, 3H), 1.90-1.71 (m, 8H), 1.61-1.51 (m, 4H), 1.06-0.98 (m, 6H). Mass (m/z): 333.3 [M+H]⁺. HPLC: Rt=3.329 mins (Agilent, poroshell 120, SB—C18 2.7 μm, 4.6×50 mm, ACN/Water (0.1% FA)=5%-5%-95%-95%-95%-5%, 0 min-0.5 min-10 min-10.5 min-12.0 min). 552B: ¹H NMR (400 MHZ, Methanol-d₄) δ 7.03 (br, 1H), 6.56 (br, 1H), 4.26-4.10 (m, 1H), 3.90-3.72 (m, 2H), 3.53-3.41 (m, 2H), 3.24-3.07 (m, 3H), 2.29 (s, 3H), 2.18-2.02 (m, 4H), 1.64-1.47 (m, 6H), 1.41-1.30 (m, 2H), 1.09-0.94 (m, 6H). Mass (m/z): 333.3 [M+H]⁺. HPLC: Rt=3.195 mins (Agilent, poroshell 120, SB—C18 2.7 μm, 4.6×50 mm, ACN/Water (0.1% FA)=5%-5%-95%-95%-95%-5%, 0 min-0.5 min-10 min-10.5 min-12.0 min).

### Compound 553

### N2-(6-(2,6-dimethylmorpholino)-2-methylpyridin-3-yl)bicyclo[2.2.2]octane-2,5-diamine

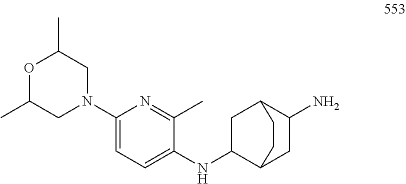 [SMILES: Cc1nc(N2CC(C)OC(C)C2)ccc1NC1CC2CCC1CC2N]

The title compound 553 (44.0 mg) was prepared in a total yield of 51.2% as a white solid according to the procedure for compound 24. ¹H NMR (400 MHz, DMSO-d₆) δ 6.84 (d, J=8.8 Hz, 1H), 6.51 (d, J=8.8 Hz, 1H), 4.16 (m, 1H), 3.95-3.77 (m, 2H), 3.67-3.54 (m, 2H), 3.25 (m, 1H), 2.40-2.30 (m, 2H), 2.25 (s, 3H), 2.21-2.13 (m, 2H), 2.08-1.95 (m, 2H), 1.86-1.63 (m, 3H), 1.52-1.27 (m, 3H), 1.12 (d, J=6.0 Hz, 6H). Mass (m/z): 345.3 [M+H]⁺.

### Compound 554

### 1-(4-(5-((6-aminospiro[3.3]heptan-2-yl)amino)-6-methylpyridin-2-yl)-2,6-dimethylpiperazin-1-yl)-2,2,2-trifluoroethan-1-one

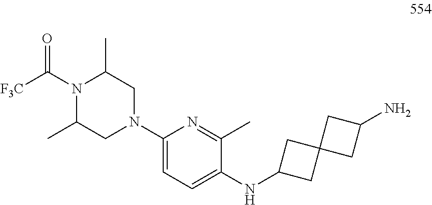 [SMILES: Cc1nc(N2CC(C)N(C(=O)C(F)(F)F)C(C)C2)ccc1NC1CC2(CC(N)C2)C1]

The title compound 554 as a yellow solid (23 mg, 28.5% yield) was prepared from tert-butyl (6-((6-(3,5-dimethyl-4-(2,2,2-trifluoroacetyl) piperazin-1-yl)-2-methylpyridin-3-yl)amino)spiro [3.3]heptan-2-yl)carbamate (0.1 g, 0.2 mmol), DCM (5 mL) and TFA (1 mL) according to the procedure for 24. ¹H NMR (400 MHZ, CD₃OD) δ 6.86 (d, J=8.6 Hz, 1H), 6.60 (d, J=8.6 Hz, 1H), 4.59 (s, 1H), 4.27 (s, 1H), 3.92 (d, J=11.6 Hz, 2H), 3.71-3.73 (m, 1H), 3.47-3.49 (m, 1H), 2.82-2.84 (m, 2H), 2.59-2.35 (m, 3H), 2.33-2.25 (m, 4H), 1.97-1.99 (m, 4H), 1.43 (d, J=22.2 Hz, 6H). Mass (m/z): 425.7 [M+H]⁺.

### Compound 555

### 1-(4-(5-((6-aminospiro[3.3]heptan-2-yl)amino)-6-methylpyridin-2-yl)-2,6-dimethylpiperazin-1-yl)ethan-1-one

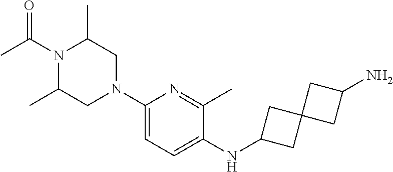 [SMILES: CC(=O)N1C(C)CN(c2ccc(NC3CC4(CC(N)C4)C3)c(C)n2)CC1C]

The title compound 555 (34.8 mg) was prepared in a total yield of 77.9% as a yellow solid from tert-butyl (6-((6-(4-acetyl-3,5-dimethylpiperazin-1-yl)-2-methylpyridin-3-yl)amino)spiro[3.3]heptan-2-yl) carbamate (56.6 mg, 0.12 mmol), TFA according to the procedure for compound 24. ¹H NMR (400 MHZ, DMSO-d₆) δ 6.77 (d, J=8.4 Hz, 1H), 6.60 (d, J=8.4 Hz, 1H), 4.78-4.26 (m, 2H), 3.95-3.81 (m, 2H), 3.65 (m, 1H), 3.54 (m, 1H), 2.75-2.60 (m, 2H), 2.44-2.28 (m, 2H), 2.22 (s, 3H), 2.20-2.13 (m, 4H), 2.04 (s, 3H), 1.97-1.85 (m, 2H), 1.22 (d, J=6.4 Hz, 6H). Mass (m/z): 372.2 [M+H]⁺

### Compound 556

### N2-(6-(4,4-dimethyl-1,4-azasilinan-1-yl)-2-methylpyridin-3-yl)spiro[3.3]heptane-2,6-diamine

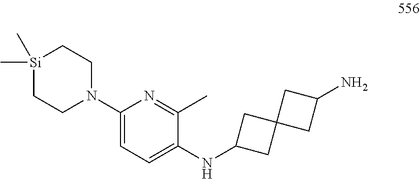 [SMILES: Cc1nc(N2CC[Si](C)(C)CC2)ccc1NC1CC2(CC(N)C2)C1]

The title compound 556 (17.4 mg, 24%) as a yellow solid was prepared from tert-butyl (6-((6-(4,4-dimethyl-1,4-azasilinan-1-yl)-2-methylpyridin-3-yl)amino)spiro[3.3]heptan-2-yl)carbamate (95 mg, 0.21 mmol), HCl in dioxane (5 mL) and DCM (5 mL) according to the procedure for 37. ¹H NMR (400 MHZ, CD₃OD) δ 6.88 (d, J=8.8 Hz, 1H), 6.53 (d, J=8.7 Hz, 1H), 3.82-3.64 (m, 5H), 3.55-3.45 (m, 1H), 2.60-2.46 (m, 2H), 2.43-2.37 (m, 1H), 2.33-2.22 (m, 4H), 2.07-1.91 (m, 4H), 0.79-0.71 (m, 4H), 0.07 (s, 6H). Mass (m/z): 345.3 [M+H]⁺.

### Compound 557

### N3-(6-aminospiro [3.3]heptan-2-yl)-6-methyl-N2-(2,2,2-trifluoroethyl)pyridine-2,5-diamine

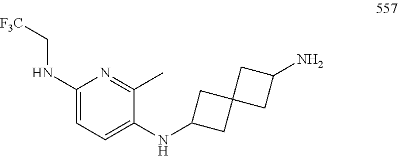 [SMILES: Cc1nc(NCC(F)(F)F)ccc1NC1CC2(CC(N)C2)C1]

The title compound 557 (22.2 mg, 28.2%) as a white solid was prepared from tert-butyl (5-((6-((tert-butoxycarbonyl)amino)spiro[3.3]heptan-2-yl)amino)-6-methylpyridin-2-yl) (2,2,2-t rifluoroethyl)carbamate (129 mg, 0.2502 mmol), DCM (10 mL) and TFA (1 mL) according to the procedure for 24. ¹H NMR (400 MHZ, CD₃OD) δ 6.88 (d, J=8.7 Hz, 1H), 6.41 (d, J=8.5 Hz, 1H), 3.99 (q, J=9.5 Hz, 2H), 3.70 (t, J=7.6 Hz, 1H), 3.29 (d, J=8.4 Hz, 1H), 2.56-2.42 (m, 2H), 2.36 (ddd, J=11.6, 6.8, 5.1 Hz, 1H), 2.27 (s, 3H), 2.27-2.23 (m, 1H), 1.96-1.78 (m, 4H). Mass (m/z): 315.1 [M+H]⁺.

### Compound 558

### N⁵-(6-aminospiro [3.3]heptan-2-yl)-N2,6-dimethyl-N2-(2,2,2-trifluoroethyl)pyridine-2,5-diamine

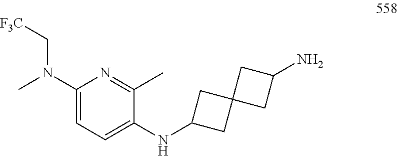 [SMILES: Cc1nc(N(C)CC(F)(F)F)ccc1NC1CC2(CC(N)C2)C1]

The title compound 558 (13.1 mg, 9.8%) as a yellow solid was prepared from tert-butyl (6-((2-methyl-6-(methyl (2,2,2-trifluoroethyl)amino)pyridin-3-yl)amino)spiro[3.3]heptan-2-yl)carbamate (165 mg, 0.3851 mmol), DCM (10 mL) and TFA (1 mL) according to the procedure for 24. ¹H NMR (400 MHZ, CD₃OD) δ 6.92 (d, J=8.7 Hz, 1H), 6.47 (d, J=8.7 Hz, 1H), 4.29 (q, J=9.5 Hz, 2H), 3.72 (p, J=7.6 Hz, 1H), 3.32-3.28 (m, 1H), 3.03 (s, 3H), 2.58-2.42 (m, 2H), 2.37 (ddd, J=11.7, 6.9, 5.0 Hz, 1H), 2.29 (s, 3H), 2.28-2.23 (m, 1H), 1.97-1.78 (m, 4H). Mass (m/z): 329.3 [M+H]⁺.

### Compound 559

### N-(((1r, 4r)-4-aminocyclohexyl)methyl)-4-(1-(2,2,2-trifluoroethyl)piperidin-4-yl)aniline

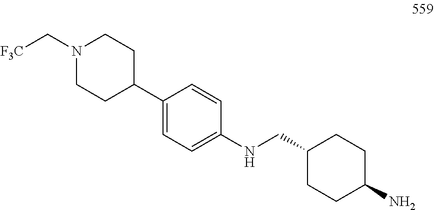 [SMILES: N[C@H]1CC[C@H](CNc2ccc(C3CCN(CC(F)(F)F)CC3)cc2)CC1]

The title compound 559 (75.1 mg, 75%) as a white solid was prepared from tert-butyl ((1r,4r)-4-(((4-(1-(2,2,2-trifluoroethyl)piperidin-4-yl)phenyl)amino)methyl)cyclohexyl)carbam ate (127 mg, 0.27 mmol) and HCl in dioxane (5 mL) and DCM (5 mL) according to the procedure for 37. ¹H NMR (400 MHZ, CD₃OD) δ 6.98 (d, J=8.5 Hz, 2H), 6.57 (d, J=8.6 Hz, 2H), 3.13-3.01 (m, 4H), 2.93 (d, J=6.7 Hz, 2H), 2.90-2.81 (m, 1H), 2.50-2.40 (m, 2H), 2.40-2.31 (m, 1H), 2.06-1.87 (m, 4H), 1.78-1.68 (m, 4H), 1.63-1.53 (m, 1H), 1.32-1.22 (m, 2H), 1.15-1.02 (m, 2H). Mass (m/z): 370.2 [M+H]⁺.

### Compound 560

### N2-(4-(1-(2,2, 2-trifluoroethyl)piperidin-4-yl)phenyl)spiro[3.3]heptane-2,6-diamine

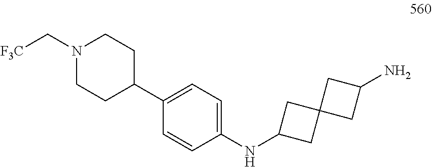 [SMILES: NC1CC2(C1)CC(Nc1ccc(C3CCN(CC(F)(F)F)CC3)cc1)C2]

The title compound 560 (103.3 mg, 98%) as a yellow solid was prepared from tert-butyl (6-((4-(1-(2,2,2-trifluoroethyl)piperidin-4-yl)phenyl)amino)spiro[3.3]heptan-2-yl)carbamate (122 mg, 0.26 mmol), HCl in dioxane (5 mL) and DCM (5 mL) according to the procedure for 37. ¹H NMR (400 MHZ, CD₃OD) δ 6.98 (d, J=8.5 Hz, 2H), 6.53 (d, J=8.5 Hz, 2H), 3.82-3.71 (m, 1H), 3.55-3.41 (m, 1H), 3.13-3.00 (m, 4H), 2.58-2.24 (m, 7H), 2.08-1.82 (m, 4H), 1.77-1.65 (m, 4H). Mass (m/z): 368.2 [M+H]⁺.

### Compound 561

### N1-(6-(4-(trifluoromethyl)piperidin-1-yl)naphthalen-2-yl)cyclohexane-1,4-diamine

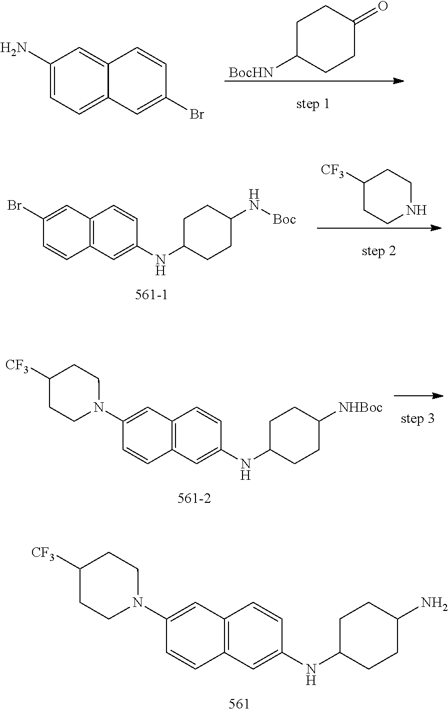 [SMILES: Nc1ccc2cc(Br)ccc2c1 ; CC(C)(C)OC(=O)NC1CCC(=O)CC1 ; CC(C)(C)OC(=O)NC1CCC(Nc2ccc3cc(Br)ccc3c2)CC1 ; FC(F)(F)C1CCNCC1 ; CC(C)(C)OC(=O)NC1CCC(Nc2ccc3cc(N4CCC(C(F)(F)F)CC4)ccc3c2)CC1 ; NC1CCC(Nc2ccc3cc(N4CCC(C(F)(F)F)CC4)ccc3c2)CC1]

Step 1. Preparation of tert-butyl (4-((6-bromonaphthalen-2-yl)amino)cyclohexyl)carbamate (561-1)

A mixture of 6-bromonaphthalen-2-amine (300 mg, 1.35 mmol), tert-butyl (4-oxocyclohexyl)carbamate (345 mg, 1.62 mmol), NaBH₃CN (169 mg, 2.7 mmol) in MeOH (20 mL), CH₃COOH (0.1 mL) was stirred at 50° C. for 2h. It was quenched with H₂O (30 mL) and extracted with EA (3×30 mL), and the organic layers were concentrated to afford product (0.4 g, yield: 70.4%) as a yellow solid. Mass (m/z): 419.2 [M+H]⁺.

Step 2. Preparation of tert-butyl (4-((6-(4-(trifluoromethyl)piperidin-1-yl) naphthalen-2-yl)amino)cyclohexyl)carbamate (561-2)

A mixture of tert-butyl (4-((6-bromonaphthalen-2-yl)amino)cyclohexyl)carbamate (200 mg, 0.47 mmol), 4-(trifluoromethyl)piperidine (88 mg, 0.57 mmol), RuPhos (44 mg, 0.09 mmol), Pd₂(dba)₃ (87 mg, 0.095 mmol), and Cs₂CO₃ (465 mg, 1.42 mmol) in toluene (20 mL) was stirred at 110° C. for 3h. It was quenched with H₂O (30 mL) and extracted with EA (3×30 mL), the organic layers were concentrated to afford product (0.2 g, 85%) as a yellow solid. Mass (m/z): 491.7 [M+H]⁺.

Step 3. Preparation of N1-(6-(4-(trifluoromethyl)piperidin-1-yl)naphthalen-2-yl)cyclohexane-1,4-diamine (561)

To a solution of 8 (200 mg, 0.21 mmol) in 1,4-dioxane (3 mL) was added HCl/1,4-dioxane (1 ml). Then the mixture was stirred overnight at 25° C. Solvent was removed under vacuum and the residue was purified by prep-HPLC (column-Gemini-C18 150×21.2 mm, 5 μm; Mobile phase: ACN-H₂O (0.1% FA), 5%-20%) to afford the desired product (33.5 mg, 21.0%) as a yellow solid. ¹H NMR (400 MHZ, CD₃OD) δ 7.87-7.67 (m, 3H), 7.46 (m, 1H), 7.41-7.10 (m, 2H), 3.86 (t, J=14.7 Hz, 10H), 3.86 (m, 2H), 3.70s (m, 1H), 3.1 (m, 1H), 2.37-1.75 (m, 12H), 2.04 (m, 2H). Mass (m/z): 491.7 [M−H].

### Compound 562

### N-(2-(4-(trifluoromethyl)piperidin-1-yl)quinolin-6-yl)cyclohexane-1,4-diamine

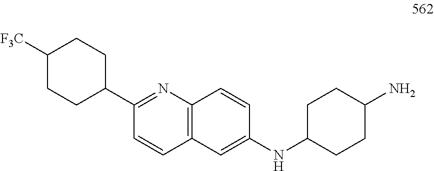 [SMILES: NC1CCC(Nc2ccc3nc(C4CCC(C(F)(F)F)CC4)ccc3c2)CC1]

The title compound 562A (41.9 mg, 32.5%) as a yellow solid and 562B as a yellow solid (36.4 mg, 28.2%) were prepared from tert-butyl (4-((2-(4-(trifluoromethyl)piperidin-1-yl) quinolin-6-yl)amino)cyclohexyl)carbamate (305 mg, 0.6179 mmol), 1.4-dioxane (10 mL) and HCl/1,4-dioxane (10 mL) according to the procedure for 24. 562A: ¹H NMR (400 MHZ, DMSO-d₆) δ 7.82 (d, J=9.1 Hz, 1H), 7.36 (d, J=9.0 Hz, 1H), 7.10 (d, J-9.2 Hz, 1H), 7.01 (dd, J-9.0, 2.5 Hz, 1H), 6.66 (d, J-2.4 Hz, 1H), 4.47 (d, J=13.2 Hz, 2H), 3.19 (dd, J=15.1, 6.5 Hz, 1H), 2.98 (t, J=11.2 Hz, 1H), 2.84 (t, J=11.9 Hz, 2H), 2.66-2.54 (m, 1H), 2.15-1.82 (m, 6H), 1.54-1.38 (m, 4H), 1.31-1.13 (m, 2H). Mass (m/z): 392.8 [M+H]⁺. HPLC: Rt: 3.067 min (Column: XBRIDGE 2.1*50 mm, 3.5 μm; Mobile Phase: H₂O (0.05% TFA)/ACN (0.05% TFA), ACN from 0% to 60% over 7 minutes, 7-8 min, ACN from 60% to 100%; 0.8 mL/min). 562B: ¹H NMR (400 MHz, DMSO-d₆) δ 7.80 (d, J=9.1 Hz, 1H), 7.37 (d, J=9.0 Hz, 1H), 7.25-6.96 (m, 2H), 6.64 (d, J=2.2 Hz, 1H), 5.65 (s, 1H), 4.47 (d, J=13.0 Hz, 2H), 3.51 (s, 1H), 3.05 (s, 1H), 2.84 (t, J=11.9 Hz, 2H), 1.83 (dd, J=34.8, 16.2 Hz, 6H), 1.75-1.54 (m, 4H), 1.45 (qd, J=12.5, 3.9 Hz, 2H). Mass (m/z): 392.8 [M+H]⁺. HPLC: Rt: 3.391 min (Column: XBRIDGE 2.1*50 mm, 3.5 μm; Mobile Phase: H₂O (0.05% TFA)/ACN (0.05% TFA), ACN from 0% to 60% over 7 minutes, 7-8 min, ACN from 60% to 100%; 0.8 mL/min).

### Compound 563

### 4-((6-(4-(trifluoromethyl)piperidin-1-yl)naphthalen-2-yl)amino)cyclohexane-1-carboxamide

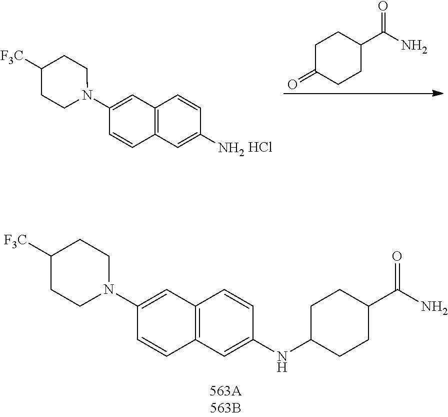 [SMILES: Nc1ccc2cc(N3CCC(C(F)(F)F)CC3)ccc2c1 ; Cl ; NC(=O)C1CCC(=O)CC1 ; NC(=O)C1CCC(Nc2ccc3cc(N4CCC(C(F)(F)F)CC4)ccc3c2)CC1]

A mixture of 6-(4-(trifluoromethyl)piperidin-1-yl) naphthalen-2-amine hydrochloride (100 mg, 0.3014 mmol), 4-oxocyclohexane-1-carboxamide (42.55 mg, 0.3014 mmol) and sodium triacetoxyborohydride (191.64 mg, 0.9042 mmol) in DCE (20 mL) was stirred overnight at 25° C. After cooling, the excess DCE was removed under vacuum and the residue was extracted three times with DCM (30 mL) and water (30 mL). Organic layer was combined, solvent was removed under vacuum and the crude was purified through prep-HPLC (column-Gemini-C18 150×21.2 mm, 5 μm; Mobile phase: ACN-H₂O (0.1% FA), 20%-50%) to afford the desired product 563A as a gray solid (9.5 mg, 7.1%) and 563B as a purple solid (9.9 mg, 7.4%). 563A: ¹H NMR (400 MHZ, CD₃OD) δ 7.39 (dd, J=8.9, 2.5 Hz, 2H), 7.07 (dd, J=9.0, 2.4 Hz, 1H), 7.00 (d, J=2.3 Hz, 1H), 6.81 (dd, J=8.8, 2.3 Hz, 1H), 6.70 (d, J=2.1 Hz, 1H), 3.63 (d, J=12.3 Hz, 2H), 3.28-3.22 (m, 1H), 2.59 (td, J=12.2, 2.0 Hz, 2H), 2.26-2.07 (m, 4H), 1.94-1.79 (m, 4H), 1.66 (tt, J-12.5, 6.4 Hz, 2H), 1.59-1.47 (m, 2H), 1.14 (dt, J-13.1, 10.5 Hz, 2H). HPLC: Rt: 4.428 min (Column: XBRIDGE 2.1*50 mm, 3.5 μm; Mobile Phase: H₂O (0.05% TFA) ACN (0.05% TFA), ACN from 0% to 60% over 7 minutes, 7-8 min, ACN from 60% to 100%; 0.8 mL/min). 563B ¹H NMR (400 MHZ, CD₃OD) δ 7.39 (dd, J-8.8, 2.1 Hz, 2H), 7.08 (dd, J=9.0, 2.4 Hz, 1H), 7.01 (d, J=2.3 Hz, 1H), 6.88 (dd, J=8.8, 2.3 Hz, 1H), 6.69 (d, J=2.1 Hz, 1H), 3.64 (d, J=11.7 Hz, 2H), 3.59 (s, 1H), 2.61 (dd, J=12.3, 10.0 Hz, 2H), 2.32-2.17 (m, 2H), 1.91 (d, J=11.7 Hz, 2H), 1.85-1.75 (m, 4H), 1.73-1.54 (m, 6H).Mass (m/z): 419.7 [M+H]⁺. HPLC: Rt: 4.598 min (Column: XBRIDGE 2.1*50 mm, 3.5 μm; Mobile Phase: H₂O (0.05% TFA) ACN (0.05% TFA), ACN from 0% to 60% over 7 minutes, 7-8 min, ACN from 60% to 100%; 0.8 mL/min).

### Compound 564

### N²-(6-(2,6-dimethylmorpholino)naphthalen-2-yl)spiro[3.3]heptane-2,6-diamine

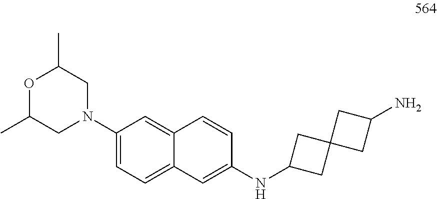 [SMILES: CC1CN(c2ccc3cc(NC4CC5(CC(N)C5)C4)ccc3c2)CC(C)O1]

The title compound N²-(6-(2,6-dimethylmorpholino) naphthalen-2-yl)spiro[3.3]heptane-2,6-diamine 564 (41.8 mg, 53%) as an off-white solid was prepared from tert-butyl (6-((6-(2,6-dimethylmorpholino) naphthalen-2-yl)amino)spiro[3.3]heptan-2-yl)carbamate (100 mg, 0.21 mmol), DCM (3 mL) and TFA (1 mL) according to the procedure for 24. ¹H NMR (400 MHZ, DMSO-d₆) δ 7.41 (t, J=8.3 Hz, 2H), 7.14 (dd, J=9.0, 2.5 Hz, 1H), 6.93 (d, J=2.4 Hz, 1H), 6.79 (dd, J=8.8, 2.3 Hz, 1H), 6.47 (d, J=2.3 Hz, 1H), 5.76 (d, J=6.6 Hz, 1H), 3.77-3.64 (m, 3H), 3.55-3.47 (m, 2H), 3.22-3.09 (m, 1H), 2.50-2.49 (m, 1H), 2.43-2.41 (m, 1H), 2.37-2.27 (m, 2H), 2.24-2.16 (m, 2H), 2.14-2.05 (m, 1H), 2.03-1.88 (m, 1H), 1.85-1.72 (m, 2H), 1.71-1.57 (m, 2H), 1.13 (d, J=6.3 Hz, 6H). MS (m/z): 366.3 [M+H]⁺.

### Compound 565

### N2-(2-((2S,6R)-2,6-dimethylmorpholino) quinolin-6-yl)spiro[3.3]heptane-2,6-diamine

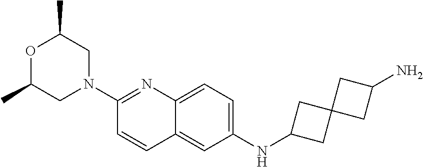 [SMILES: C[C@@H]1CN(c2ccc3cc(NC4CC5(CC(N)C5)C4)ccc3n2)C[C@H](C)O1]

The title compound 565 (20.2 mg) was prepared in a total yield of 84.6% as a light yellow solid from tert-butyl (6-((2-((2S,6R)-2,6-dimethylmorpholino) quinolin-6-yl)amino)spiro[3.3]heptan-2-yl)carbamate (30.0 mg, 0.065 mmol), TFA according to the procedure for compound 24. ¹H NMR (400 MHZ, DMSO-d₆) δ 7.81 (d, J=9.2 Hz, 1H), 7.36 (d, J=8.8 Hz, 1H), 7.09 (d, J=9.2 Hz, 1H), 6.95 (dd, J=9.2, 2.4 Hz, 1H), 6.49 (d, J=2.4 Hz, 1H), 4.27-4.11 (m, 2H), 3.73 (m, 1H), 3.67-3.56 (m, 2H), 3.24 (m, 1H), 2.42-2.31 (m, 5H), 2.17 (m, 1H), 1.92-1.75 (m, 4H), 1.17 (d, J=6.4 Hz, 6H). Mass (m/z): 367.2 [M+H]⁺

### Compound 566

### 1-(4-(5-((6-aminospiro[3.3]heptan-2-yl)amino)-6-methylpyridin-2-yl)-2,6-dimethylpiperazin-1-yl)-2, 2, 2-trifluoroethan-1-one

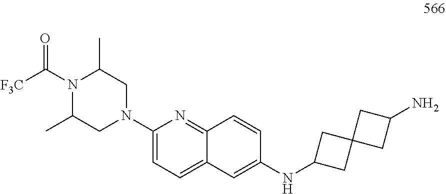 [SMILES: CC1CN(c2ccc3cc(NC4CC5(CC(N)C5)C4)ccc3n2)CC(C)N1C(=O)C(F)(F)F]

The title compound 566 (15 mg, 18.3%) as a yellow solid was prepared from tert-butyl (6-((2-(3,5-dimethyl-4-(2,2,2-trifluoroacetyl) piperazin-1-yl) quinolin-6-yl)amino)spiro[3.3]heptan-2-yl)carbamate (0.1 g, 0.2 mmol), DCM (5 mL) and TFA (1 mL) according to the procedure for 24. ¹H NMR (400 MHZ, CD₃OD) δ 7.82 (d, J=9 Hz, 1H), 7.45 (d, J=9.0 Hz, 1H), 7.08 (d, J=9.0 Hz, 1H), 7.00 (dd, J=9.0, 2.5 Hz, 1H), 6.60 (d, J=2.4 Hz, 1H), 4.63 (s, 1H), 4.32-4.31 (m, 3H), 3.83-3.81 (m, 1H), 3.46-3.45 (m, 1H), 3.11 (d, J=11.8 Hz, 2H), 2.64-2.41 (m, 3H), 2.36-2.24 (m, 1H), 2.07-1.87 (m, 4H), 1.42 (d, J=19.8 Hz, 6H). Mass (m/z): 461.7 [M+H]⁺.

### Compound 567

### N2-(2-(3,5-dimethyl-4-(2,2,2-trifluoroethyl)piperazin-1-yl) quinolin-6-yl)spiro-[3.3]heptane-2,6-diamine

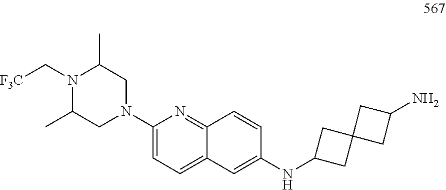 [SMILES: CC1CN(c2ccc3cc(NC4CC5(CC(N)C5)C4)ccc3n2)CC(C)N1CC(F)(F)F]

The title compound 567 (31.6 mg) as a white solid was prepared from tert-butyl (6-((2-(3,5-dimethyl-4-(2,2,2-trifluoroethyl) piperazin-1-yl) quinolin-6-yl)amino)spiro[3.3]hepta n-2-yl)carbamate (100 mg, 0.18 mmol), DCM (10 mL) and TFA (1 mL) according to the procedure for 24. ¹HNMR (400 MHZ, DMSO-d₆) δ 7.76 (d, J=9.2 Hz, 1H), 7.31 (d, J=9.0 Hz, 1H), 7.07 (d, J=9.2 Hz, 1H), 6.91 (dd, J=9.0, 2.6 Hz, 1H), 6.45 (d, J=2.6 Hz, 1H), 5.79 (d, J=6.4 Hz, 1H), 4.13 (d, J=12.0 Hz, 2H), 3.69 (d, J=7.0 Hz, 1H), 3.33 (t, J=8.0 Hz, 3H), 3.27-3.22 (m, 1H), 2.76-2.69 (m, 2H), 2.56-2.48 (m, 3H), 2.37-2.29 (m, 2H), 2.16-2.07 (m, 1H), 1.76 (ddd, J=14.2, 10.8, 2.6 Hz, 3H), 1.09 (d, J=6.2 Hz, 6H). Mass (m/z): 448.2 [M+H]⁺.

### Compound 568

### N2-(6-(2,6-dimethylthiomorpholino)-2-methylpyridin-3-yl)spiro[3.3]heptane-2,6-diamine

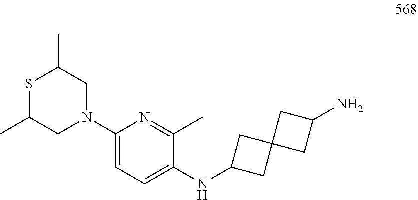 [SMILES: Cc1nc(N2CC(C)SC(C)C2)ccc1NC1CC2(CC(N)C2)C1]

The title compound 568 (8.1 mg, 98.4%) as a white solid was prepared from tert-butyl (6-((6-(2,6-dimethylthiomorpholino)-2-methylpyridin-3-yl)amino)-spiro [3.3]heptan-2-yl)carba mate (100 mg, 0.22 mmol), DCM (10 mL) and TFA (1 mL) according to the procedure for 24. ¹HNMR (400 MHZ, CD₃OD) δ 6.89 (d, J=8.8 Hz, 1H), 6.55 (d, J=8.8 Hz, 1H), 4.24 (dd, J=13.0, 2.6 Hz, 2H), 3.77-3.69 (m, 1H), 3.29 (dd, J=8.6, 7.4 Hz, 1H), 3.06 (ddd, J=10.6, 6.8, 2.8 Hz, 2H), 2.60-2.50 (m, 3H), 2.48-2.34 (m, 2H), 2.29 (s, 3H), 2.28-2.18 (m, 1H), 1.97-1.78 (m, 4H), 1.16 (d, J=6.8 Hz, 6H). Mass (m/z): 347.2 [M+H]⁺.

### Compound 569

### N²-(2-methyl-6-thiomorpholinopyridin-3-yl) spiro[3.3]heptane-2,6-diamine

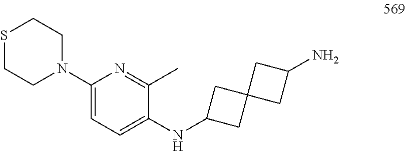 [SMILES: Cc1nc(N2CCSCC2)ccc1NC1CC2(CC(N)C2)C1]

The desired product 569 (10.3 mg, 12.85%) as an oil was prepared from tert-butyl (6-((2-methyl-6-thiomorpholinopyridin-3-yl)amino)spiro[3.3]heptan-2-yl)carbamate (100 mg, 0.2389 mmol), DCM (10 mL), and TFA (1 mL) according to the procedure for 568. ¹H NMR (400 MHZ, CD₃OD) δ 6.77 (d, J=8.7 Hz, 1H), 6.44 (d, J=8.7 Hz, 1H), 3.61 (p, J=7.6 Hz, 1H), 3.55-3.47 (m, 4H), 3.26-3.15 (m, 1H), 2.60-2.53 (m, 4H), 2.46-2.23 (m, 3H), 2.17 (s, 3H), 2.14 (dd, J=6.7, 5.4 Hz, 1H), 1.86-1.67 (m, 4H). Mass (m/z): 318.9 [M+H]⁺.

### Compound 570

### N2-(7-(2,6-dimethylmorpholino) isoquinolin-3-yl)spiro[3.3]heptane-2,6-diamine

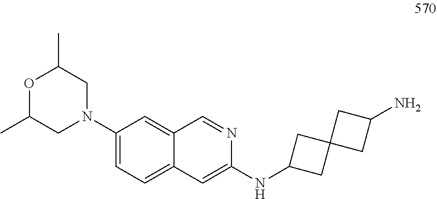 [SMILES: CC1CN(c2ccc3cc(NC4CC5(CC(N)C5)C4)ncc3c2)CC(C)O1]

The title compound 570 (9.3 mg) as a white solid was prepared from tert-butyl (6-((7-(2,6-dimethylmorpholino) isoquinolin-3-yl)amino)spiro[3.3]heptan-2-yl)carbamate (150 mg, 0.21 mmol), TFA (2 mL) and DCM (10 mL) according to the procedure for 24. H NMR (400 MHZ, DMSO-d₆) δ 8.67 (s, 1H), 7.48 (d, J=9.2 Hz, 1H), 7.41 (dd, J=9.2, 2.2 Hz, 1H), 7.06 (s, 1H), 6.95 (d, J=7.6 Hz, 1H), 6.39 (d, J=4.4 Hz, 1H), 6.27 (t, J=8.4 Hz, 1H), 3.96 (dd, J=14.8, 7.2 Hz, 1H), 3.83 (dd, J=16.0, 8.0 Hz, 1H), 3.76-3.70 (m, 2H), 3.58 (d, J=10.8 Hz, 2H), 3.24-3.13 (m, 1H), 2.38-2.30 (m, 2H), 2.28-2.22 (m, 2H), 2.14 (s, 1H), 1.97-1.92 (m, 1H), 1.90-1.85 (m, 2H), 1.74-1.61 (m, 1H), 1.17 (d, J=6.2 Hz, 6H). Mass (m/z): 367.3 [M+H]⁺.

### Compound 571

### N²-(2-(2,6-dimethylmorpholino) quinazolin-6-yl)spiro[3.3]heptane-2,6-diamine

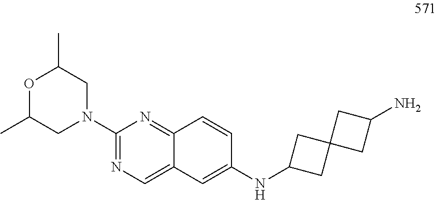 [SMILES: CC1CN(c2ncc3cc(NC4CC5(CC(N)C5)C4)ccc3n2)CC(C)O1]

The title compound 571 (47.3 mg, 83.6%) was prepared from tert-butyl (6-((2-(2,6-dimethylmorpholino) quinazolin-6-yl)amino)spiro[3.3]heptan-2-yl)carbamate (72 mg, 0.15 mmol), 5 mL of a solution of HCl in 1,4-dioxane according to the procedure for 37. 1¹H NMR (400 MHZ, DMSO-d₆) δ 8.97 (s, 1H), 8.10 (s, 2H), 7.41-7.31 (m, 2H), 7.26-7.15 (m, 1H), 6.57 (s, 1H), 6.17 (s, 1H), 4.53 (dd, J=13.3, 2.4 Hz, 2H), 3.76 (t, J=7.6 Hz, 1H), 3.63-3.52 (m, 3H), 2.62-2.56 (m, 1H), 2.53-2.52 (m, 2H), 2.48-2.40 (m, 2H), 2.29-2.10 (m, 3H), 1.94-1.83 (m, 2H), 1.17 (d, J=6.2 Hz, 6H). Mass (m/z): 368.2 [M+H]⁺.

### Compound 572

### N1-(3-(4-(trifluoromethyl)piperidin-1-yl)quinolin-7-yl)cyclohexane-1,4-diamine

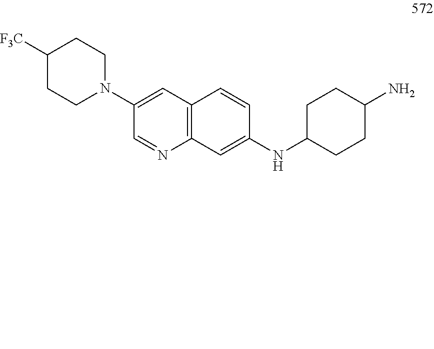 [SMILES: NC1CCC(Nc2ccc3cc(N4CCC(C(F)(F)F)CC4)cnc3c2)CC1]

The title compound 572 (10.1 mg) as a white solid was prepared from tert-butyl (4-((3-(4-(trifluoromethyl)piperidin-1-yl) quinolin-7-yl)amino)cyclohexyl)carbamate (100 mg, 0.21 mmol) and HCl in 1,4-Dio. (10 mL, 4N) according to the procedure for 37. ¹H NMR (400 MHz, DMSO-d₆) δ 8.58 (d, J=2.8 Hz, 1H), 7.51 (d, J=8.8 Hz, 1H), 7.42 (d, J=2.8 Hz, 1H), 7.08 (d, J=8.8 Hz, 1H), 6.76 (s, 1H), 5.96 (s, 1H), 3.76 (d, J=12.2 Hz, 2H), 3.56 (s, 1H), 3.10 (s, 1H), 2.72 (t, J=11.4 Hz, 2H), 2.54 (s, 1H), 1.99-1.55 (m, 13H). Mass (m/z): 393.2 [M+H]⁺.

### Compound 573

### N1-(2-(4-(trifluoromethyl)piperidin-1-yl)quinoxalin-6-yl)cyclohexane-1,4-diamine

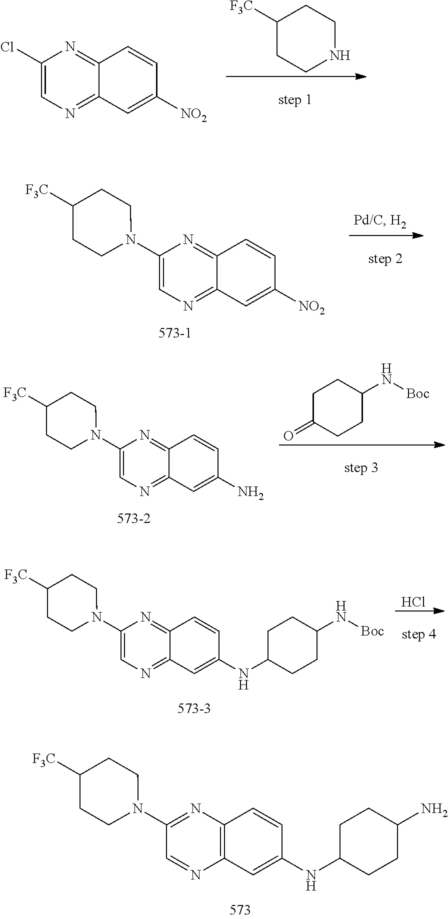

Step 1. Preparation of 1-(3-nitrophenyl)-4-(trifluoromethyl)piperidine (573-1)

A mixture of 2-chloro-6-nitroquinoxaline (0.2 g, 0.95 mmol), 4-(trifluoromethyl)piperidine (0.15 g. 0.95 mmol), Cs₂CO₃ (617 mg, 1.9 mmol) and DMF (10 mL) was stirred at 100° C. for 3 h. The mixture was diluted with EA (400 mL) and washed with water (300 mL×3). The organic phase was concentrated and purified by flash, eluted with PE: EA=10:1 to 1:1 to give the desired product (0.33 g, 92.0%) as a yellow solid. Mass (m/z): 326.8 [M+H]⁺.

Step 2. Preparation of 2-(4-(trifluoromethyl)piperidin-1-yl) quinoxalin-6-amine (573-2)

To a solution of 1-(3-nitrophenyl)-4-(trifluoromethyl)piperidine (0.33 g, 1 mmol) and Pd/C (80 mg) in THF (10 mL) was stirred under H₂ at 25° C. for 3 h. The organic phase was concentrated and purified by flash, eluted with DCM: MeOH=10:1 to 5:1 to give the desired product as a yellow oil (0.3 g, 99%). Mass (m/z): 297.2 [M+H]⁺.

Step 3. Preparation of tert-butyl (4-((3-(4-(trifluoromethyl)piperidin-1-yl)phenyl)amino)cyclohexyl)carbamate (573-3)

A mixture of 2-(4-(trifluoromethyl)piperidin-1-yl)quinoxalin-6-amine (0.3 g, 1 mmol), tert-butyl (4-oxocyclohexyl)carbamate (0.2 g, 1 mmol), NaBH(OAc)₃ (0.63 g, 3 mmol) and DCE (10 mL). The reaction was stirred for 3 h at R.T. Quenched with water (10 mL), the reaction was extracted by EA (10 mL) for 3 times. The combined organic layers were dried over sodium sulfate, concentrated and purified by flash, eluted with PE/EA=10:1 to 1:1 to give the desired product as a yellow oil (0.3 g, 60.8%). Mass (m/z): 493.7 [M-tBu+H]⁺.

Step 4. Preparation of N1-(2-(4-(trifluoromethyl)piperidin-1-yl) quinoxalin-6-yl)cyclohexane-1,4-diamine (573):

To a solution of tert-butyl tert-butyl (4-((3-(4-(trifluoromethyl)piperidin-1-yl)phenyl)amino)cyclohexyl)carbamate (300 mg, 0.61 mmol) in DCM (5 ml) was added TFA (1 mL) and the mixture was stirred for 2 h. Quenched with NaHCO₃ (10 mL), the reaction was extracted by EA (10 mL) for 3 times. The combined organic layers were dried over sodium sulfate, removed under vacuum and the residue was purified by preparative (column-Gemini-C18 150×21.2 mm, 5 μm; Mobile phase: ACN-H₂O (0.1% FA), 10%-30%) to afford 573A (102.9 mg) as a yellow solid and 573B (107.6 mg) as a yellow solid. 573A: ¹H NMR (400 MHZ, CD₃OD) δ 8.75 (s, 1H), 7.71 (d, J-9.2, 1H), 7.50-7.40 (m, 1H), 7.25 (d, J-2.4, 1H), 4.66 (d, J-13.6, 2H), 3.60-3.44 (m, 1H), 3.25-3.09 (m, 3H), 2.65-2.55 (m, 1H), 2.29-2.14 (m, 4H), 2.07 (d, J=10.9, 2H), 1.77-1.41 (m, 6H). Mass (m/z): 394.2 [M+H]⁺. HPLC: Rt=3.886 min (Column: XBRIDGE 3.5 μm, 2.1*50 mm; Mobile phase: H₂O (0.05% TFA)-ACN (0.05% TFA), ACN from 0 to 60% over 7 minutes, Flow rate: 0.8 mL/min). 573B: ¹H NMR (400 MHZ, CD₃OD) δ 8.72 (s, 1H), 7.68 (d, J=9.2, 1H), 7.44 (dd, J=9.2, 2.4, 1H), 7.02 (d, J=2.4, 1H), 4.62 (d, J=13.6, 2H), 3.73 (s, 1H), 3.21-3.15 (m, 2H), 2.65-2.55 (m, 1H), 2.12-1.82 (m, 11H), 1.76-1.65 (m, 2H). Mass (m/z): 394.2 [M+H]⁺. HPLC: Rt=4.058 min (Column: XBRIDGE 3.5 μm, 2.1*50 mm; Mobile phase: H₂O (0.05% TFA)-ACN (0.05% TFA), ACN from 0 to 60% over 7 minutes, Flow rate: 0.8 mL/min).

### Compound 574

### N²-(3-methyl-4-(4-(2,2,2-trifluoroethyl)piperazin-1-yl)phenyl)spiro/3.3/heptane-2.6-diamine

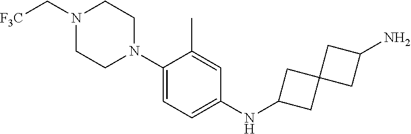 [SMILES: Cc1cc(NC2CC3(CC(N)C3)C2)ccc1N1CCN(CC(F)(F)F)CC1]

The titled compound 574 (26.1 mg, 51.3%) as a white solid was prepared according to the procedure outlined for compound 24. ¹H NMR (400 MHZ, DMSO-d₆) δ 8.38 (s, 3H), 6.81 (d, J=8.4 Hz, 1H), 6.33 (d, J=2.8 Hz, 1H), 6.27 (d, J=8.4 Hz, 1H), 5.68-5.26 (m, 1H), 3.64 (d, J=7.2 Hz, 1H), 3.57-3.47 (m, 1H), 3.21 (q, J=10.3 Hz, 2H), 2.71 (s, 7H), 2.39-2.30 (m, 2H), 2.23-2.15 (m, 3H), 2.13 (s, 3H), 1.86-1.76 (m, 2H). Mass (m/z): 383.2 [M+H]⁺.

### Compound 575

### 6-((6-((2S,6R)-2,6-diethylmorpholino)-2-methylpyridin-3-yl)amino)spiro[3.3]heptane-2-carboxamide

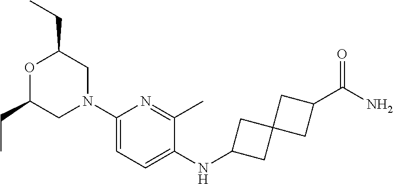 [SMILES: CC[C@@H]1CN(c2ccc(NC3CC4(C3)CC(C(N)=O)C4)c(C)n2)C[C@H](CC)O1]

The titled compound 575 (9.3 mg, 23.5%) as a light-yellow solid was prepared according to the procedure outlined for compound 5. ¹H NMR (400 MHZ, DMSO-d₆) δ 7.10 (s, 2H), 6.73 (d, J=8.7 Hz, 1H), 6.64 (s, 1H), 6.50 (d, J=8.7 Hz, 1H), 4.58-4.41 (m, 1H), 3.84 (d, J=11.9 Hz, 2H), 3.65-3.56 (m, 1H), 3.44-3.37 (m, 3H), 2.91-2.81 (m, 1H), 2.35-2.07 (m, 9H), 2.01-1.95 (m, 1H), 1.94-1.86 (m, 1H), 1.83-1.75 (m, 1H), 1.54-1.43 (m, 4H), 0.94 (t, J=7.4 Hz, 6H). Mass (m/z): 387.3 [M+H]⁺.

### Compound 576

### N1-(6-((2S,6R)-2,6-diethylmorpholino)-2-methylpyridin-3-yl)cyclobutane-1,3-diamine

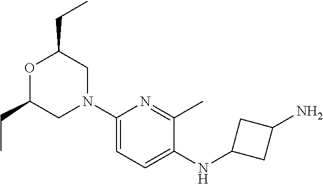 [SMILES: CC[C@@H]1CN(c2ccc(NC3CC(N)C3)c(C)n2)C[C@H](CC)O1]

The titled compound 576 (4.2 mg, 15.5%) as a light-yellow solid was prepared according to the procedure outlined for compound 24. ¹H NMR (400 MHZ, Methanol-d₄) δ 6.79 (d, J=8.7 Hz, 1H), 6.55 (d, J=8.9 Hz, 1H), 4.16-4.03 (m, 1H), 3.98-3.90 (m, 1H), 3.85-3.75 (m, 2H), 3.54-3.44 (m, 2H), 2.61-2.49 (m, 2H), 2.33 (s, 3H), 1.67-1.50 (m, 4H), 1.39-1.26 (m, 4H), 1.02 (t, J=7.5 Hz, 6H). Mass (m/z): 319.2 [M+H]⁺.

### Compound 577

### (3aR,6aS)-N2-(6-((3R,5S)-3,5-dimethyl-4-(2.2,2-trifluoroethyl)piperazin-1-yl)-2-methylpyridin-3-yl)octahydropentalene-2,5-diamine

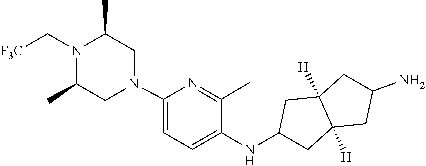 [SMILES: Cc1nc(N2C[C@@H](C)N(CC(F)(F)F)[C@@H](C)C2)ccc1NC1C[C@H]2CC(N)C[C@H]2C1]

The title compound 577 (13.5 mg) was prepared in a two steps total yield of 17.5% as a white solid with 1:0.1 mixture by ¹H NMR from 6-((3R,5S)-3,5-dimethyl-4-(2,2,2-trifluoroethyl) piperazin-1-yl)-2-methylpyridin-3-amine and (3as,6as)-tetrahydropentalene-2,5 (1H,3H)-dione according to the procedure for 24. ¹H NMR (400 MHZ, DMSO-d₆) δ 8.06 (s, 3H), 7.92 (s, 1H), 6.58 (s, 1H), 4.02 (s, 1H), 3.84 (s, 3H), 2.74 (s, 5H), 2.23 (s, 4H), 1.73 (d, J=24.9 Hz, 4H), 1.39 (s, 3H), 1.10 (d, J=6.3 Hz, 9H). Mass (m/z): 426.4 [M+H]⁺.

### Compound 578

### N-(3-((1s,3R)-3-aminocyclobutyl)propyl)-6-((2S,6R)-2,6-dimethylmorpholino)-2-methylpyridin-3-amine

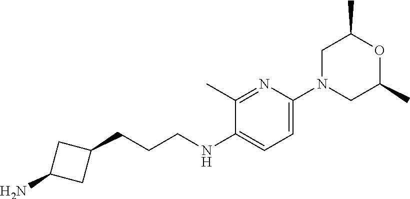 [SMILES: Cc1nc(N2C[C@@H](C)O[C@@H](C)C2)ccc1NCCC[C@H]1C[C@@H](N)C1]

The titled compound 578 (61.9 mg, 63.7%) as a white solid was prepared according to the procedure outlined for compound 23. ¹H NMR (400 MHZ, DMSO-d₆) δ 6.82 (d, J=8.8 Hz, 1H), 6.51 (d, J=8.8 Hz, 1H), 4.32 (s, 1H), 3.83 (d, J=11.2 Hz, 2H), 3.64-3.57 (m, 2H), 2.92 (s, 2H), 2.26 (d, J=7.6 Hz, 1H), 2.20 (s, 3H), 2.18-2.11 (m, 2H), 1.82-1.66 (m, 1H), 1.46-1.37 (m, 4H), 1.23 (d, J=6.4 Hz, 2H), 1.14 (d, J=6.4 Hz, 6H). Mass (m/z): 332.9 [M+H]⁺.

### Compound 579

### N2-(6-(3,5-dimethyl-4-(2,2,2-trifluoroethyl) piperazin-1-yl)-2-fluoropyridin-3-yl)spiro[3.3]heptane-2,6-diamine

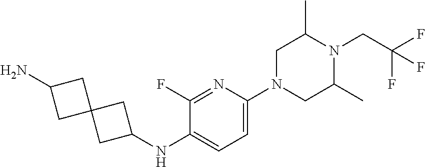 [SMILES: CC1CN(c2ccc(NC3CC4(CC(N)C4)C3)c(F)n2)CC(C)N1CC(F)(F)F]

The titled compound 579 (37.6 mg, 50%) as brown oil was prepared according to the procedure outlined for compound 24. ¹H NMR (400 MHZ, DMSO-d₆) δ 6.99-6.94 (m, 1H), 6.53 (d, J=8.4, 1H), 4.96 (d, J=6.0, 1H), 3.74 (d, J=11.2, 2H), 3.66-3.61 (m, 1H), 3.38-3.30 (m, 2H), 3.20-3.13 (m, 1H), 2.74-2.72 (m, 2H), 2.39-2.28 (m, 5H), 2.26-2.17 (m, 2H), 2.16-2.04 (m, 1H), 1.87-1.80 (m, 2H), 1.69-1.57 (m, 2H), 1.09 (d, J=6.4, 6H). Mass (m/z): 416.2 [M+H]⁺.

### Compound 580

N²-(6-(3.5-dimethyl-4-(2,2,2-trifluoroethyl) piperazin-1-yl)-5-fluoropyridin-3-yl)spiro[3.3]hept ane-2,6-diamine

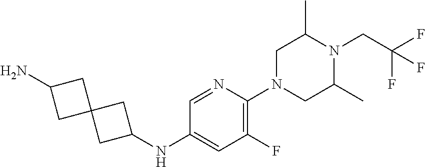 [SMILES: CC1CN(c2ncc(NC3CC4(CC(N)C4)C3)cc2F)CC(C)N1CC(F)(F)F]

The titled compound 580 (105.6 mg, 43.68%) as a yellow solid was prepared according to the procedure outlined for compound 24. ¹H NMR (400 MHZ, CD₃OD) δ 7.37 (d, J=2.0 Hz, 1H), 6.76 (dd, J=14.0, 2.4 Hz, 1H), 3.73 (dt, J=15.6, 7.6 Hz, 1H), 3.50-3.30 (m, 4H), 2.95 (br, 2H), 2.63-2.32 (m, 7H), 1.94-1.80 (m, 4H), 1.16 (d, J=6.4 Hz, 6H). Mass (m/z): 415.8 [M+H]⁺.

### Compound 581

### N-(((1r,4r)-4-aminocyclohexyl)methyl)-2-methyl-6-(2-oxa-9-azaspiro [5.5]undecan-9-yl)pyridin-3-amine

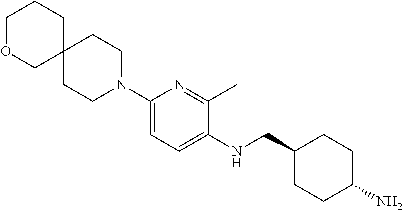 [SMILES: Cc1nc(N2CCC3(CCCOC3)CC2)ccc1NC[C@H]1CC[C@H](N)CC1]

The titled compound 581 (39.1 mg, 12.7%) as a white solid was prepared according to the procedure outlined for compound 24. ¹H NMR (400 MHZ, DMSO-d₆) δ 6.79 (d, J=8.8 Hz, 1H), 6.49 (d, J=8.8 Hz, 1H), 4.30 (s, 1H), 3.53 (t, J=4.8 Hz, 2H), 3.34 (s, 2H), 3.22 (t, J=5.2 Hz, 4H), 2.81 (s, 2H), 2.46 (d, J=3.2 Hz, 1H), 2.20 (s, 3H), 1.79-1.73 (m, 4H), 1.51-1.37 (m, 9H), 1.01-0.85 (m, 4H). Mass (m/z): 373.3 [M+H]⁺.

### Compound 582

### N-(((1r,4r)-4-aminocyclohexyl)methyl)-2-methyl-6-(3-oxa-9-azaspiro [5.5]undecan-9-yl)pyridin-3-amine

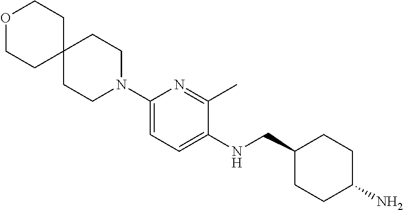 [SMILES: Cc1nc(N2CCC3(CCOCC3)CC2)ccc1NC[C@H]1CC[C@H](N)CC1]

The titled compound 582 (33.60 mg, 28.42%) as a white solid was prepared according to the procedure outlined for compound 24. ¹H NMR (400 MHZ, CD₃OD) δ 6.84 (d, J=8.8 Hz, 1H), 6.49 (d, J=8.8 Hz, 1H), 3.61-3.57 (m, 4H), 3.18-3.11 (m, 4H), 2.83 (d, J=6.8 Hz, 2H), 2.53-2.44 (m, 1H), 2.19 (s, 3H), 1.84-1.75 (m, 4H), 1.59-1.54 (m, 4H), 1.48-1.41 (m, 5H), 1.12-1.04 (m, 4H). Mass (m/z): 372.9 [M+H]⁺.

### Compound 583

### N-(2-((1r,3S)-3-aminocyclobutyl)ethyl)-6-((2S,6R)-2,6-dimethylmorpholino) naphthalen-2-amine

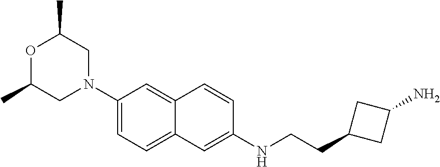 [SMILES: C[C@@H]1CN(c2ccc3cc(NCC[C@H]4C[C@H](N)C4)ccc3c2)C[C@H](C)O1]

The titled compound 583 as a yellow solid was prepared according to the procedure outlined for compound 78. ¹H NMR (400 MHZ, CD₃OD) δ 7.50 (dd, J=8.8, 5.6 Hz, 2H), 7.18 (dd, J=9.0, 2.4 Hz, 1H), 7.05 (d, J=2.3 Hz, 1H), 6.91 (dd, J=8.8, 2.3 Hz, 1H), 6.74 (d, J=2.2 Hz, 1H), 3.89-2.79 (m, 2H), 3.60-3.55 (m, 2H), 3.51 (d, J=10.8 Hz, 2H), 3.14-3.04 (m, 2H), 2.47-2.28 (m, 3H), 2.12-1.96 (m, 4H), 1.83 (dd, J=14.6, 7.6 Hz, 2H), 1.62-1.57 (m, 1H), 1.24 (d, J=6.3 Hz, 6H). Mass (m/z): 353.8 [M+H]⁺.

### Compound 584

### 6-((6-(4-(trifluoromethyl)piperidin-1-yl)naphthalen-2-yl)amino)spiro[3.3]heptane-2-carboxamide

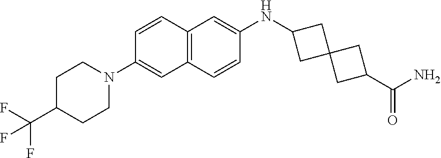 [SMILES: NC(=O)C1CC2(CC(Nc3ccc4cc(N5CCC(C(F)(F)F)CC5)ccc4c3)C2)C1]

The title compound 584 as white solid (27.4 mg, 20%) was prepared according to the procedure outlined for compound 10. ¹H NMR (400 MHZ, CD₃OD) δ 7.49 (dd, J=8.8, 4.7 Hz, 2H), 7.17 (dd, J=9.0, 2.4 Hz, 1H), 7.09 (d, J=2.2 Hz, 1H), 6.88 (dd, J=8.8, 2.3 Hz, 1H), 6.66 (d, J=2.0 Hz, 1H), 3.90-3.79 (m, 1H), 3.72 (d, J=12.3 Hz, 2H), 3.08-2.97 (m, 1H), 2.71-2.60 (m, 3H), 2.51-2.41 (m, 1H), 2.33 (d, J=8.6 Hz, 2H), 2.31-2.19 (m, 2H), 2.17-2.09 (m, 1H), 2.03-1.94 (m, 3H), 1.86 (dd, J=11.4, 8.0 Hz, 1H), 1.80-1.66 (m, 2H). Mass (m/z): 432.2 [M+H]⁺.

### Compound 585

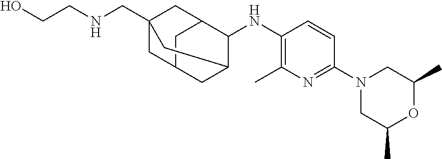 [SMILES: Cc1nc(N2C[C@@H](C)O[C@@H](C)C2)ccc1NC1C2CC3CC1CC(CNCCO)(C3)C2]

The titled compound 585 (51.2 mg, 55.6%) as a light-yellow solid was prepared according to the procedure outlined for compound 24. ¹H NMR (400 MHZ, DMSO-d₆) δ 9.42 (s, 1H), 8.51 (s, 2H), 7.01-6.74 (m, 1H), 6.67-6.34 (m, 1H), 5.35-5.20 (m, 1H), 4.42-4.28 (m, 1H), 3.91-3.82 (m, 3H), 3.76-3.68 (m, 2H), 3.64-3.55 (m, 2H), 3.47-3.37 (m, 2H), 3.15 (d, J=12.5 Hz, 1H), 3.04-2.94 (m, 2H), 2.69 (d, J=13.4 Hz, 2H), 2.37-2.28 (m, 3H), 2.24-2.14 (m, 2H), 2.06-1.90 (m, 4H), 1.77-1.66 (m, 3H), 1.62-1.53 (m, 2H), 1.44-1.34 (m, 3H), 1.16-1.06 (m, 9H). Mass (m/z): 429.3 [M+H]⁺.

### Compound 586

### N-(((1r,4r)-4-aminocyclohexyl)methyl)-2-fluoro-4-(4-(2,2,2-trifluoroethyl) piperazin-1-yl)aniline

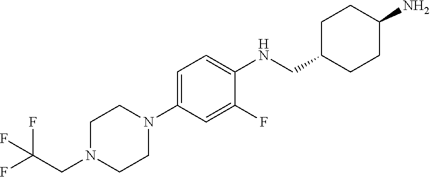 [SMILES: N[C@H]1CC[C@H](CNc2ccc(N3CCN(CC(F)(F)F)CC3)cc2F)CC1]

The titled compound 586 (5.2 mg, 19.8%) as a white solid was prepared according to the procedure outlined for compound 24. ¹H NMR (400 MHZ, DMSO-d₆) δ 6.71 (d, J=14.4 Hz, 1H), 6.57 (d, J=5.2 Hz, 2H), 4.77 (d, J=6.4 Hz, 1H), 3.25-3.17 (m, 2H), 2.95 (t, J=4.8 Hz, 4H), 2.83 (t, J=6.8 Hz, 2H), 2.72 (t, J=4.8 Hz, 4H), 1.74 (d, J=9.2 Hz, 4H), 1.52-1.39 (m, 2H), 1.00-0.87 (m, 4H). Mass (m/z): 389.2 [M+H]⁺.

### Compound 587

### N2-(6-(7-azabicyclo[2.2.1]heptan-7-yl)-2-methylpyridin-3-yl)spiro[3.3]heptane-2,6-diamine

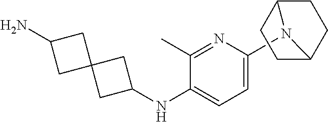 [SMILES: Cc1nc(N2C3CCC2CC3)ccc1NC1CC2(CC(N)C2)C1]

The titled compound 587 (28.5 mg, 54.4%) as a yellow solid was prepared according to the procedure outlined for compound 24. ¹H NMR (400 MHZ, DMSO-d₆) δ 8.38 (s, 3H), 6.80 (d, J=8.4 Hz, 2H), 4.89 (s, 1H), 4.45 (s, 2H), 3.72-3.46 (m, 2H), 2.43-2.28 (m, 4H), 2.26-2.14 (m, 3H), 1.96 (d, J=10.4 Hz, 2H), 1.68-1.60 (m, 3H), 1.40 (d, J=7.6 Hz, 3H). Mass (m/z): 313.3 [M+H]⁺.

### Compound 588

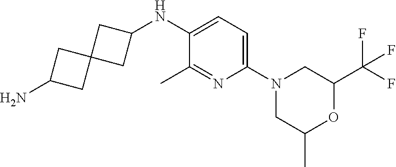 [SMILES: Cc1nc(N2CC(C)OC(C(F)(F)F)C2)ccc1NC1CC2(CC(N)C2)C1]

The titled compound 588 (16.2 mg, 52.3%) as a light-yellow solid was prepared according to the procedure outlined for compound 24. ¹H NMR (400 MHZ, DMSO-d₆) δ 8.07 (br s, 2H), 6.76 (d, J=8.4 Hz, 1H), 6.56 (d, J=8.4 Hz, 1H), 4.61 (br s, 1H), 4.53-4.41 (m, 1H), 4.21-4.10 (m, 1H), 3.72-3.62 (m, 1H), 3.60-3.47 (m, 2H), 3.45-3.37 (m, 2H), 3.00-2.89 (m, 1H), 2.44-2.30 (m, 2H), 2.22 (s, 3H), 2.20-2.07 (m, 4H), 1.98-1.86 (m, 2H), 1.22 (d, J=6.4 Hz, 3H). Mass (m/z): 385.2 [M+H]⁺.

### Compound 589

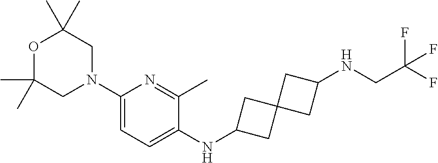 [SMILES: Cc1nc(N2CC(C)(C)OC(C)(C)C2)ccc1NC1CC2(CC(NCC(F)(F)F)C2)C1]

The titled compound 589 (17.6 mg, 34.1%) as a light-yellow solid was prepared according to the procedure outlined for compound 23. ¹H NMR (400 MHZ, Chloroform-d) δ 6.76 (d, J=8.4 Hz, 1H), 6.42 (d, J=8.4 Hz, 1H), 3.74 (p, J=7.6 Hz, 1H), 3.29 (p, J=7.6 Hz, 1H), 3.19 (s, 4H), 3.10 (q, J=9.6 Hz, 2H), 2.59-2.49 (m, 1H), 2.47-2.38 (m, 2H), 2.29 (s, 3H), 2.28-2.22 (m, 1H), 1.89-1.73 (m, 4H), 1.29 (s, 12H). Mass (m/z): 441.3 [M+H]⁺.

### Compound 590

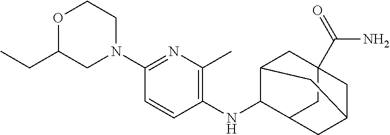 [SMILES: CCC1CN(c2ccc(NC3C4CC5CC3CC(C(N)=O)(C5)C4)c(C)n2)CCO1]

The titled compound 590 (10.0 mg, 31.2%) as a white solid was prepared according to the procedure outlined for compound 10.1¹H NMR (400 MHZ, DMSO-d₆) δ 6.99 (s, 1H), 6.88 (d, J=8.7 Hz, 1H), 6.73 (s, 1H), 6.54 (d, J=8.7 Hz, 1H), 3.94-3.83 (m, 3H), 3.75 (d, J=12.3 Hz, 1H), 3.54 (t, J=11.4 Hz, 1H), 3.44-3.35 (m, 2H), 2.36-2.21 (m, 5H), 2.01-1.93 (m, 4H), 1.91-1.80 (m, 5H), 1.78-1.71 (m, 2H), 1.53-1.36 (m, 4H), 0.93 (t, J=7.5 Hz, 3H). Mass (m/z): 399.3 [M+H]⁺.

### Compound 591

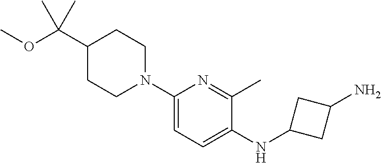 [SMILES: COC(C)(C)C1CCN(c2ccc(NC3CC(N)C3)c(C)n2)CC1]

The titled compound 591 (20.8 mg, 46.2%) as a white solid was prepared according to the procedure outlined for compound 24. ¹H NMR (400 MHZ,) δ 6.83-6.74 (m, 1H), 6.64-6.56 (m, 1H), 4.18-3.84 (m, 4H), 3.21 (s, 3H), 2.67-2.48 (m, 4H), 2.47-2.26 (m, 5H), 1.82-1.71 (m, 2H), 1.68-1.56 (m, 1H), 1.51-1.37 (m, 2H), 1.15 (s, 6H). Mass (m/z): 333.4 [M+H]⁺.

### Compound 592

### N1-(2-(4-(trifluoromethyl)piperidin-1-yl)quinolin-6-yl)cyclobutane-1,3-diamine

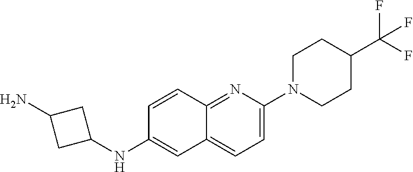 [SMILES: NC1CC(Nc2ccc3nc(N4CCC(C(F)(F)F)CC4)ccc3c2)C1]

The titled compound 592 (15.9 mg, 36.1%) as a yellow solid was prepared according to the procedure outlined for compound 24. ¹H NMR (400 MHZ, DMSO-d₆) δ 7.80 (d, J=9.2 Hz, 1H), 7.37 (d, J=8.4 Hz, 1H), 7.10 (d, J=9.2 Hz, 1H), 6.98 (d, J=9.2 Hz, 1H), 6.41 (s, 1H), 5.98-5.74 (m, 1H), 4.47 (d, J=13.2 Hz, 2H), 3.94-3.73 (m, 1H), 3.60-3.46 (m, 1H), 2.84 (t, J=12.8 Hz, 2H), 2.73-2.52 (m, 3H), 2.06 (q, J=8.4 Hz, 3H), 1.93-1.73 (m, 3H), 1.47 (t, J=12.8 Hz, 2H). Mass (m/z): 365.2 [M+H]⁺.

### Compound 593

### N¹-(6-(4-(2,2,2-trifluoroethyl)piperazin-1-yl)naphthalen-2-yl)cyclobutane-1,3-diamine

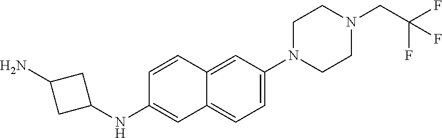 [SMILES: NC1CC(Nc2ccc3cc(N4CCN(CC(F)(F)F)CC4)ccc3c2)C1]

The titled compound 593 (6.5 mg, 15.9%) as a white solid was prepared according to the procedure outlined for compound 24. ¹H NMR (400 MHZ, DMSO-d₆) δ 8.50 (s, 3H), 7.67-7.52 (m, 1H), 7.47 (d, J=8.0 Hz, 1H), 7.15 (d, J=8.8 Hz, 1H), 7.00 (s, 1H), 6.88 (d, J=8.8 Hz, 1H), 6.51 (s, 1H), 6.18-6.02 (m, 1H), 4.17 (s, 1H), 3.77 (s, 1H), 3.24 (t, J=10.4 Hz, 2H), 3.13 (s, 2H), 2.79 (s, 3H), 2.23 (s, 2H), 2.12-1.97 (m, 1H). Mass (m/z): 379.3 [M+H]⁺.

### Compound 594

### N²-(6-(4-(2,6-dimethyltetrahydro-2H-pyran-4-yl)piperidin-1-yl)-2-methylpyridin-3-yl)spiro[3.3]heptane-2,6-diamine

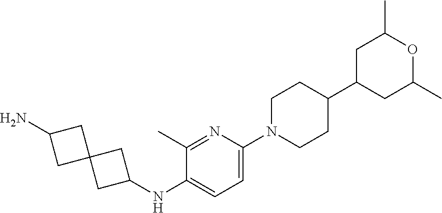 [SMILES: Cc1nc(N2CCC(C3CC(C)OC(C)C3)CC2)ccc1NC1CC2(CC(N)C2)C1]

The titled compound 594 (3.1 mg, 19.25%) as a white solid was prepared according to the procedure outlined for compound 24. ¹H NMR (400 MHZ, CD₃OD) δ 6.88 (d, J=8.8 Hz, 1H), 6.58 (d, J=8.8 Hz, 1H), 3.97 (d, J=12.4 Hz, 2H), 3.80-3.67 (m, 2H), 3.50 (dd, J=9.6, 6.4 Hz, 2H), 2.65-2.56 (m, 4H), 2.50-2.44 (m, 1H), 2.44-2.35 (m, 1H), 2.29 (s, 3H), 2.23-2.17 (m, 3H), 2.02 (d, J=10.8 Hz, 2H), 1.83 (d, J=12.6 Hz, 2H), 1.75-1.71 (m, 2H), 1.32 (s, 3H), 1.19 (d, J=6.2 Hz, 6H), 0.90-0.86 (m, 3H). Mass (m/z): 413.4 [M+H]⁺.

### Compound 595

### N⁵-(((1r,4r)-4-aminocyclohexyl)methyl)-N²-(tert-butyl)-6-methylpyridine-2,5-diamine

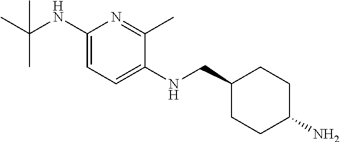 [SMILES: Cc1nc(NC(C)(C)C)ccc1NC[C@H]1CC[C@H](N)CC1]

The titled compound 595 (65.2 mg, 33.3%) as a black solid was prepared according to the procedure outlined for compound 24. ¹H NMR (400 MHZ, DMSO-d₆) δ 6.71 (d, J=8.8, 1H), 6.27 (d, J=8.8, 1H), 4.95 (s, 1H), 3.98 (s, 1H), 2.76 (d, J=6.4, 2H), 2.49-2.40 (m, 1H), 2.17 (s, 3H), 1.79-1.75 (m, 4H), 1.43-1.31 (m, 1H), 1.31 (s, 9H), 1.04-0.83 (m, 4H). Mass (m/z): 290.9 [M+H]⁺.

### Compound 596

### N⁵-(6-aminospiro [3.3]heptan-2-yl)-N²-(tert-butyl)-6-methylpyridine-2,5-diamine

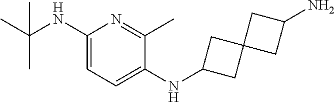 [SMILES: Cc1nc(NC(C)(C)C)ccc1NC1CC2(CC(N)C2)C1]

The titled compound 596 (101.7 mg, 58%) as a black solid was prepared according to the procedure outlined for compound 24. ¹H NMR (400 MHZ, DMSO-d₆) δ 6.60 (d, J=8.8, 1H), 6.23 (d, J=8.4, 1H), 5.00 (s, 1H), 4.12 (s, 1H), 3.56-3.52 (m, 1H), 3.19-3.12 (m, 1H), 2.39-2.35 (m, 1H), 2.33-2.27 (m, 1H), 2.26-2.19 (m, 1H), 2.15 (s, 3H), 2.12-2.08 (m, 1H), 1.83-1.77 (m, 2H), 1.67-1.59 (m, 2H), 1.30 (s, 9H). Mass (m/z): 288.9 [M+H]⁺.

### Compound 597

### N¹-(((1r,4r)-4-aminocyclohexyl)methyl)-N⁴-(tert-butyl)-2-methylbenzene-1,4-diamine

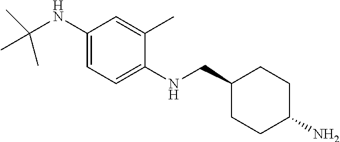 [SMILES: Cc1cc(NC(C)(C)C)ccc1NC[C@H]1CC[C@H](N)CC1]

The titled compound 597 (92.3 mg, 82.82%) as a white solid was prepared according to the procedure outlined for compound 24. ¹H NMR (400 MHZ, DMSO-d₆) δ 6.55 (dd, J=6.4, 2.4 Hz, 2H), 6.33-6.28 (m, 1H), 4.15 (s, 1H), 2.81 (d, J=6.4 Hz, 2H), 2.46 (dd, J=8.8, 5.2 Hz, 1H), 2.01 (s, 3H), 1.83-1.73 (m, 4H), 1.48 (dd, J=6.8, 3.6 Hz, 1H), 1.10 (s, 9H), 0.95 (q, J=10.4 Hz, 4H). Mass (m/z): 289.3 [M+H]⁺.

### Compound 598

### N-(((1s,4s)-4-aminocyclohexyl)methyl)-2-methyl-6-(2-methyl-6-(trifluoromethyl)morpholino)pyridin-3-amine

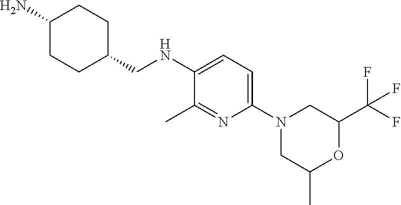 [SMILES: Cc1nc(N2CC(C)OC(C(F)(F)F)C2)ccc1NC[C@H]1CC[C@@H](N)CC1]

The titled compound 598 (15.3 mg, 42.5%) as a light-yellow solid was prepared according to the procedure outlined for compound 24. ¹H NMR (400 MHZ, DMSO-d₆) δ 7.92 (br s, 2H), 6.86 (d, J=8.4 Hz, 1H), 6.58 (d, J=8.4 Hz, 1H), 4.62-4.42 (m, 2H), 4.23-4.10 (m, 1H), 3.56-3.46 (m, 1H), 3.46-3.37 (m, 2H), 3.17 (br s, 1H), 2.97-2.86 (m, 3H), 2.23 (d, J=3.5 Hz, 3H), 1.78-1.58 (m, 5H), 1.57-1.46 (m, 4H), 1.23 (d, J=6.4 Hz, 4H). Mass (m/z): 387.2 [M+H]⁺.

### Compound 599

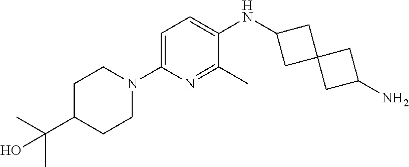 [SMILES: Cc1nc(N2CCC(C(C)(C)O)CC2)ccc1NC1CC2(CC(N)C2)C1]

The titled compound 599 (13.1 mg, 22.5%) as a light-yellow solid was prepared according to the procedure outlined for compound 24. ¹H NMR (400 MHZ, DMSO-d₆) δ 8.15 (br s, 3H), 6.85 (d, J=8.4 Hz, 1H), 6.63 (d, J=8.4 Hz, 1H), 4.71 (br s, 1H), 4.26-3.88 (m, 3H), 3.74-3.48 (m, 2H), 2.44-2.31 (m, 3H), 2.25 (s, 3H), 2.21-2.09 (m, 3H), 2.03-1.86 (m, 2H), 1.83-1.66 (m, 2H), 1.40-1.15 (m, 4H), 1.04 (s, 6H). Mass (m/z): 359.3 [M+H]⁺.

### Compound 600

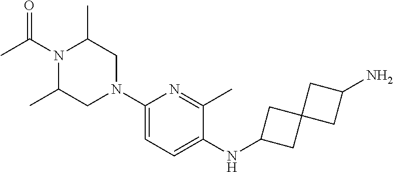 [SMILES: CC(=O)N1C(C)CN(c2ccc(NC3CC4(CC(N)C4)C3)c(C)n2)CC1C]

The titled compound 600 (25.7 mg, 42.5%) as a light-yellow solid was prepared according to the procedure outlined for compound 24. ¹H NMR (400 MHZ, DMSO-d₆) δ 8.28 (br s, 3H), 6.77 (d, J=8.4 Hz, 1H), 6.60 (d, J=8.4 Hz, 1H), 4.75-4.33 (m, 1H), 4.26-3.99 (m, 1H), 3.88 (d, J=12.3 Hz, 2H), 3.72-3.59 (m, 1H), 3.57-3.46 (m, 1H), 2.78-2.54 (m, 2H), 2.40-2.28 (m, 2H), 2.28-2.10 (m, 6H), 2.04 (s, 3H), 2.00-1.74 (m, 3H), 1.22 (d, J=6.4 Hz, 4H). Mass (m/z): 372.3 [M+H]⁺.

### Compound 601

### N2-(6-aminospiro[3.3]heptan-2-yl)-N⁶,N⁶-dimethylnaphthalene-2,6-diamine

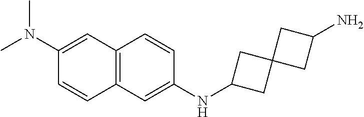 [SMILES: CN(C)c1ccc2cc(NC3CC4(CC(N)C4)C3)ccc2c1]

The titled compound 601 (38.7 mg, 43.5%) as yellow oil was prepared according to the procedure outlined for compound 24. ¹H NMR (400 MHZ, DMSO-d₆) δ 7.42 (dd, J=14.8, 9.2 Hz, 2H), 7.07 (dd, J=9.2, 2.4 Hz, 1H), 6.82-6.78 (m, 2H), 6.48 (d, J=2.0 Hz, 1H), 5.68 (d, J=6.8 Hz, 1H), 3.73 (dd, J=14.4, 7.2 Hz, 1H), 3.19 (dd, J=15.2, 7.6 Hz, 1H), 2.88 (s, 6H), 2.50-2.45 (m, 1H), 2.34 (dt, J=11.6, 6.0 Hz, 2H), 2.15-2.11 (m, 1H), 1.83-1.77 (m, 2H), 1.69-1.63 (m, 2H). Mass (m/z): 296.2 [M+H]⁺.

### Compound 602

### N2-(6-(4,4-dimethylpiperidin-1-yl)-2-methylpyridin-3-yl)spiro[3.3]heptane-2,6-diamine

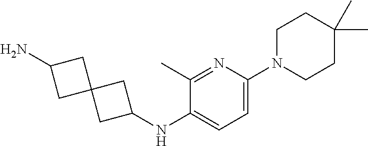 [SMILES: Cc1nc(N2CCC(C)(C)CC2)ccc1NC1CC2(CC(N)C2)C1]

The titled compound 602 (33.1 mg, 58.3%) as a yellow solid was prepared according to the procedure outlined for compound 24. ¹H NMR (400 MHZ, DMSO-d₆) δ 8.46 (s, 3H), 6.99 (s, 1H), 6.75 (s, 1H), 4.96 (s, 1H), 3.70 (s, 1H), 3.52 (s, 1H), 3.38-3.23 (m, 5H), 2.43-2.13 (m, 8H), 2.00 (s, 2H), 1.41 (s, 4H), 0.95 (s, 6H). Mass (m/z): 329.3 [M+H]⁺.

### Compound 603

### 1-(6-(2-methyl-6-(3-methyl-4-(2,2,2-trifluoroethyl) piperazin-1-yl)pyridin-3-yl)amino)spiro[3. 3]heptan-2-yl)urea

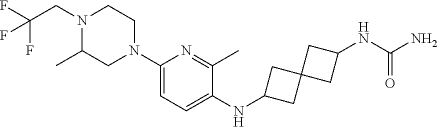 [SMILES: Cc1nc(N2CCN(CC(F)(F)F)C(C)C2)ccc1NC1CC2(CC(NC(N)=O)C2)C1]

The titled compound 603 (15.1 mg, 33.6%) as a white solid was prepared according to the procedure outlined for compound 24. ¹H NMR (400 MHZ, DMSO-d₆) δ 6.82-6.66 (m, 1H), 6.57-6.43 (m, 1H), 6.15 (d, J=8.2 Hz, 1H), 5.34 (s, 2H), 4.64-4.40 (m, 1H), 3.96-3.84 (m, 1H), 3.71-3.53 (m, 3H), 3.51-3.35 (m, 2H), 3.14-2.94 (m, 2H), 2.86-2.79 (m, 1H), 2.70-2.62 (m, 3H), 2.44-2.09 (m, 7H), 2.00-1.70 (m, 4H), 1.07 (d, J=6.2 Hz, 3H). Mass (m/z): 441.2 [M+H]⁺.

### Compound 604

### N1-(4-(3,5-dimethyl-4-(2,2,2-trifluoroethyl) piperazin-1-yl)-3-fluorophenyl)cyclobutane-1,3-diamine

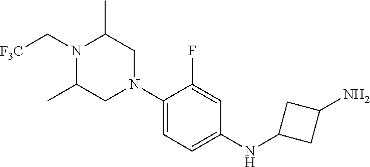 [SMILES: CC1CN(c2ccc(NC3CC(N)C3)cc2F)CC(C)N1CC(F)(F)F]

The titled compound 604 (214.9 mg, 83.3%) as a white solid was prepared according to the procedure outlined for compound 24. ¹H NMR (400 MHZ, DMSO-d₆) δ 6.81-6.77 (m, 1H), 6.25-6.18 (m, 2H), 5.85-5.76 (m, 1H), 3.79 (d, J=5.6, 1H), 3.53-3.50 (m, 1H), 3.39-3.31 (m, 2H), 3.19-3.18 (m, 1H), 2.96 (d, J=10.8, 2H), 2.84 (s, 2H), 2.62-2.58 (m, 1H), 2.35 (t, J=10.8, 2H), 2.03 (t, J=6.0, 3H), 1.52-1.44 (m, 1H), 1.05 (d, J=6.0, 6H). Mass (m/z): 374.8 [M+H]⁺

### Compound 605

### N²-(6-((2R,6S)-2,6-dimethylmorpholino)-2-methylpyridin-3-yl)-N⁶-methylspiro [3.3]heptane-2, 6-diamine

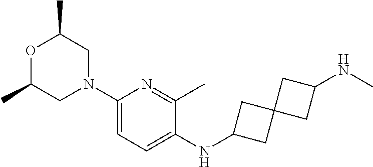

The title compound 605 (20 mg, 29%) was prepared according to the procedure outlined for compound 23. ¹H NMR (400 MHZ, CD₃OD) δ 7.47 (d, J=9.6 Hz, 1H), 7.10 (d, J=9.6 Hz, 1H), 3.93-3.58 (m, 6H), 2.71-2.45 (m, 11H), 2.39-2.37 (m, 1H), 2.23-2.21 (m, 2H), 2.13-2.03 (m, 2H), 1.23 (d, J=6.4 Hz, 6H). Mass (m/z): 344.9 [M+H]⁺.

### Compound 606

### N2-(2-methyl-6-(4-methyl-3-(trifluoromethyl) piperazin-1-yl)pyridin-3-yl)spiro[3.3]heptane-2,6-diamine

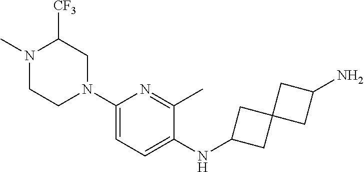 [SMILES: Cc1nc(N2CCN(C)C(C(F)(F)F)C2)ccc1NC1CC2(CC(N)C2)C1]

The titled compound 606 (20 mg, 26.1%) as a white solid was prepared according to the procedure outlined for compound 24. ¹H NMR (400 MHZ, CD₃OD) δ 6.77 (d, J=8.8 Hz, 1H), 6.46 (d, J=8.8 Hz, 1H), 3.89 (d, J=10.0 Hz, 1H), 3.64-3.61 (m, 1H), 3.52-3.35 (m, 2H), 2.95-2.81 (m, 4H), 2.50-2.17 (m, 11H), 2.00-1.78 (m, 4H). Mass (m/z): 383.8 [M+H]⁺.

### Compound 607

### 1-(4-(2-((6-(2,6-dimethylmorpholino)-2-methylpyridin-3-yl)amino)ethyl)piperazin-1-yl)ethan-1-one

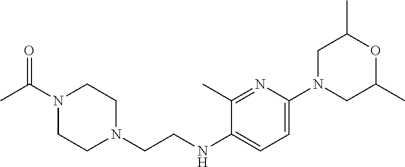 [SMILES: CC(=O)N1CCN(CCNc2ccc(N3CC(C)OC(C)C3)nc2C)CC1]

The title compound 607 (20.1 mg, 17.85% yield) as a yellow solid was prepared according to the procedure outlined for compound 24. ¹H NMR (400 MHZ, CD₃OD) δ 7.05 (d, J=8.8 Hz, 1H), 6.60 (d, J=8.8 Hz, 1H), 3.82-3.72 (m, 4H), 3.67-3.54 (m, 4H), 3.23 (t, J=6.4 Hz, 2H), 2.68 (t, J=6.4 Hz, 2H), 2.59-2.54 (m, 2H), 2.54-2.47 (m, 2H), 2.33 (s, 3H), 2.32-2.25 (m, 2H), 2.12 (s, 3H), 1.24 (d, J=6.4 Hz, 6H). Mass (m/z): 375.8 [M+H]⁺.

### Compound 608

### N1-(6-(4,4-dimethyl-1,4-azasilinan-1-yl)-2-methylpyridin-3-yl)cyclobutane-1,3-diamine

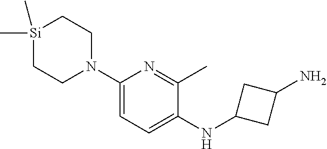 [SMILES: Cc1nc(N2CC[Si](C)(C)CC2)ccc1NC1CC(N)C1]

The title compound 608 (50.8 mg, 70%) as a yellow solid was prepared according to the procedure outlined for compound 24. ¹H NMR (400 MHZ, CD₃OD) δ 6.92 (d, J=8.8 Hz, 0.16H), 6.81 (d, J=8.8 Hz, 0.79H), 6.53 (d, J=8.7 Hz, 1H), 4.01-3.89 (m, 0.78H), 3.79-3.61 (m, 5H), 3.25-3.15 (m, 0.28H), 2.84-2.73 (m, 0.32H), 2.30 (s, 3H), 2.26-2.16 (m, 3.27H), 1.69-1.60 (m, 0.38H), 0.78-0.72 (m, 4.24H), 0.07 (s, 6H). Mass (m/z): 304.9 [M+H]⁺.

### Compound 609

### N2-(6-((2S, 6R)-2,6-dimethylmorpholino)-2-propylpyridin-3-yl)spiro[3.3]heptane-2,6-diamine

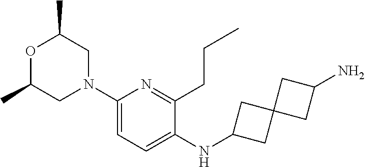 [SMILES: CCCc1nc(N2C[C@@H](C)O[C@@H](C)C2)ccc1NC1CC2(CC(N)C2)C1]

The titled compound 609 (106 mg, 59.1%) as a white solid was prepared according to the procedure outlined for compound 24. ¹H NMR (400 MHZ, CD₃OD) δ 6.83 (d, J=8.8 Hz, 1H), 6.49 (t, J=7.6 Hz, 1H), 3.79 (d, J=11.6 Hz, 2H), 3.73-3.64 (m, 3H), 3.29-3.25 (m, 1H), 2.59-2.51 (m, 2H), 2.46-2.40 (m, 2H), 2.32-2.21 (m, 4H), 1.82-1.73 (m, 6H), 1.18 (t, J=5.2 Hz, 6H), 0.96 (t, J=7.6 Hz, 3H). Mass (m/z): 359.3 [M+H]⁺.

### Compound 610

### 6-(6-(3,5-dimethyl-4-(2,2,2-trifluoroethyl) piperazin-1-yl) naphthalen-2-yl)amino)spiro[3.3]heptan-2-ol

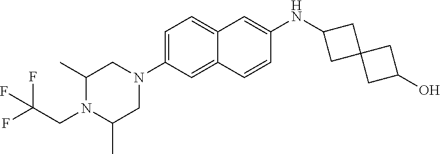 [SMILES: CC1CN(c2ccc3cc(NC4CC5(CC(O)C5)C4)ccc3c2)CC(C)N1CC(F)(F)F]

The titled compound 610 (22.5 mg, 31.2%) as a light-yellow solid was prepared according to the procedure outlined for compound 5. ¹H NMR (400 MHZ, DMSO-d₆) δ 7.44 (dd, J=8.8, 5.2 Hz, 2H), 7.17 (dd, J=8.8, 2.4 Hz, 1H), 6.96 (d, J=2.4 Hz, 1H), 6.82 (dd, J=8.8, 2.4 Hz, 1H), 6.50 (d, J=2.4 Hz, 1H), 5.79 (d, J=6.4 Hz, 1H), 4.89 (d, J=6.4 Hz, 1H), 4.06-3.92 (m, 1H), 3.81-3.67 (m, 1H), 3.52 (d, J=11.5 Hz, 2H), 3.39 (q, J=10.4 Hz, 2H), 2.96-2.78 (m, 2H), 2.48-2.29 (m, 5H), 2.26-2.12 (m, 1H), 1.92-1.75 (m, 4H), 1.12 (d, J=6.2 Hz, 6H). Mass (m/z): 448.3 [M+H]⁺.

### Compound 611

### 1-(4-(5-((6-aminospiro[3.3]heptan-2-yl)amino)-6-methylpyridin-2-yl)-2-methylpiperazin-1-yl)-2,2,2-trifluoroethan-1-one

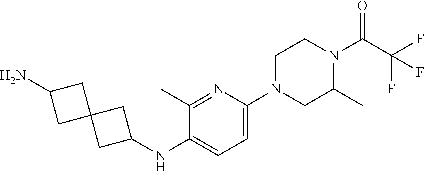 [SMILES: Cc1nc(N2CCN(C(=O)C(F)(F)F)C(C)C2)ccc1NC1CC2(CC(N)C2)C1]

The titled compound 611 (25.1 mg, 34.1%) as a light-yellow solid was prepared according to the procedure outlined for compound 24. ¹H NMR (400 MHZ, DMSO-d₆) δ 8.09 (s, 3H), 6.74 (d, J=8.7 Hz, 1H), 6.59-6.48 (m, 1H), 4.66-4.55 (m, 1H), 4.31-4.18 (m, 1H), 4.16-3.73 (m, 3H), 3.70-3.45 (m, 3H), 3.28-3.16 (m, 1H), 2.88-2.72 (m, 1H), 2.71-2.60 (m, 1H), 2.45-2.29 (m, 2H), 2.21 (s, 3H), 2.19-2.08 (m, 2H), 1.96-1.85 (m, 2H), 1.34 (d, J=6.5 Hz, 1H), 1.24 (d, J=6.5 Hz, 2H). Mass (m/z): 412.2 [M+H]⁺.

### Compound 612

### N1-(4-(3,5-dimethyl-4-(2,2,2-trifluoroethyl) piperazin-1-yl)-3-methylphenyl)cyclobutane-1,3-diamine

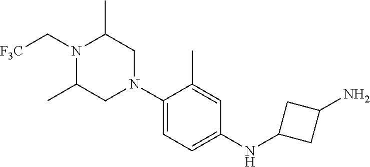 [SMILES: Cc1cc(NC2CC(N)C2)ccc1N1CC(C)N(CC(F)(F)F)C(C)C1]

The titled compound 612 (23.1 mg, 14.3%) as a white solid was prepared according to the procedure outlined for compound 24. ¹H NMR (400 MHZ, DMSO-d₆) δ=6.77 (d, J=8.4, 1H), 6.29 (d, J=2.0, 1H), 6.23-6.21 (m, 1H), 5.50-5.42 (m, 1H), 3.77 (d, J=5.2, 1H), 3.48-3.45 (m, 1H), 3.40-3.33 (m, 3H), 2.82 (s, 2H), 2.74 (d, J=11.2, 2H), 2.32 (t, J=10.8, 2H), 2.14 (s, 4H), 2.02-1.94 (m, 4H), 1.04 (d, J=6.0, 6H). Mass (m/z): 370.8 [M+H]⁺.

### Compound 613

### N²-(6-((2S, 6R)-2,6-dimethylmorpholino)-2-isopropylpyridin-3-yl)spiro[3.3]heptane-2,6-diamine

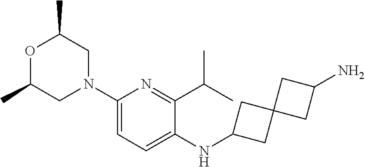 [SMILES: CC(C)c1nc(N2C[C@@H](C)O[C@@H](C)C2)ccc1NC1CC2(CC(N)C2)C1]

The titled compound 613 (1.5 mg, 2.36%) as a white solid was prepared according to the procedure outlined for compound 24. ¹H NMR (400 MHZ, CD₃OD) 6.85 (d, J=8.8 Hz, 1H), 6.48 (d, J=8.8 Hz, 1H), 3.91-3.86 (m, 2H), 3.75-3.63 (m, 3H), 3.12-3.04 (m, 1H), 2.52-2.35 (m, 2H), 2.34-2.13 (m, 5H), 2.02-1.98 (m, 1H), 1.91-1.72 (m, 4H), 1.57 (dd, J=6.8, 0.8 Hz, 1H), 1.18 (dd, J=12.4, 6.4 Hz, 12H). Mass (m/z): 358.9 [M+H]⁺.

### Compound 614

### N2-(6-morpholinonaphthalen-2-yl)spiro[3.3]heptane-2,6-diamine

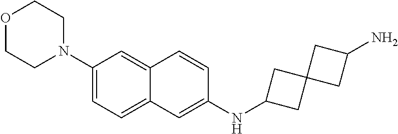 [SMILES: NC1CC2(C1)CC(Nc1ccc3cc(N4CCOCC4)ccc3c1)C2]

The titled compound 614 (8.6 mg, 5.75%) as a white solid was prepared according to the procedure outlined for compound 24. ¹HNMR (400 MHZ, CD₃OD) δ 7.47 (dd, J=8.8, 6.4 Hz, 2H), 7.15 (dd, J=9.2, 2.8 Hz, 1H), 7.04 (d, J-2.4 Hz, 1H), 6.85 (dd, J=8.8, 2.4 Hz, 1H), 6.64 (d, J=2.4 Hz, 1H), 3.86-3.82 (m, 5H), 3.32 (dd, J=11.6, 4.4 Hz, 1H), 3.14-3.11 (m, 4H), 2.60-2.54 (m, 1H), 2.49-2.38 (m, 2H), 2.28-2.21 (m, 1H), 1.86 (m, 4H). Mass (m/z): 337.9 [M+H]⁺.

### Compound 615

### 6-((6-(4,4-dimethyl-1,4-azasilinan-1-yl)-2-methylpyridin-3-yl)amino)spiro[3.3]heptane-2-carboxamide

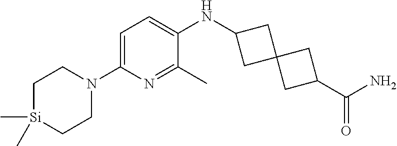 [SMILES: Cc1nc(N2CC[Si](C)(C)CC2)ccc1NC1CC2(C1)CC(C(N)=O)C2]

The title compound 615 (29.1 mg, 19%) a yellow solid was prepared according to the procedure outlined for compound 10. ¹H NMR (400 MHZ, CD₃OD) δ 6.99 (s, 1H), 6.65 (s, 1H), 3.84-3.61 (m, 3H), 3.27-3.18 (m, 0.60H), 3.08-2.96 (m, 1H), 2.64-2.55 (m, 1H), 2.45-2.36 (m, 1.51H), 2.31 (d, J=8.6 Hz, 2.51H), 2.28-2.21 (m, 1.54H), 2.17-2.08 (m, 1.40H), 2.01-1.93 (m, 1H), 1.91-1.82 (m, 1H), 1.37 (d, J=6.6 Hz, 3.50H), 0.83-0.74 (m, 4H), 0.09 (s, 6H). Mass (m/z): 373.3 [M+H]⁺.

### Compound 616

N⁵-(6-aminospiro [3.3]heptan-2-yl)-N²,6-dimethyl-N2-(2-((2,2,2-trifluoroethyl)amino)ethyl) pyri dine-2,5-diamine

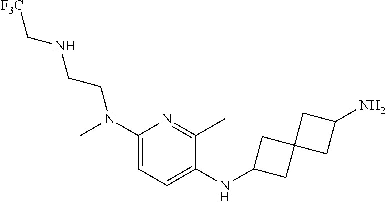 [SMILES: Cc1nc(N(C)CCNCC(F)(F)F)ccc1NC1CC2(CC(N)C2)C1]

The titled compound 616 (12.7 mg, 15.9%) as a white solid was prepared according to the procedure outlined for compound 24. ¹H NMR (400 MHZ, CDCl₃) δ 6.75 (d, J=8.8 Hz, 1H), 6.31 (d, J=8.8 Hz, 1H), 3.75-3.68 (m, 1H), 3.59 (t, J=6.0 Hz, 2H), 3.45-3.37 (m, 1H), 3.24 (q, J=9.6 Hz, 2H), 2.95-2.90 (m, 5H), 2.56-2.37 (m, 4H), 2.27 (s, 3H), 1.88-1.77 (m, 4H). Mass (m/z): 371.9 [M+H]⁺.

### Compound 617

### N-(((1r,4r)-4-aminocyclohexyl)methyl)-6-(4,4-dimethylpiperidin-1-yl)-2-methylpyridin-3-amine

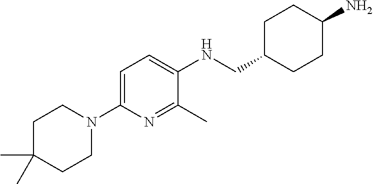 [SMILES: Cc1nc(N2CCC(C)(C)CC2)ccc1NC[C@H]1CC[C@H](N)CC1]

The titled compound 617 (28.0 mg, 29.4%) as a yellow solid was prepared according to the procedure outlined for compound 24. ¹H NMR (400 MHZ, DMSO-d₆) δ 8.33 (s, 3H), 7.08 (s, 1H), 6.77 (s, 1H), 4.89 (s, 1H), 3.35-3.26 (m, 2H), 2.89 (s, 3H), 2.34 (s, 3H), 2.00 (s, 2H), 1.86 (d, J=12.8 Hz, 2H), 1.54-1.26 (m, 7H), 1.02 (d, J=16.4 Hz, 2H), 0.95 (s, 6H). Mass (m/z): 331.3 [M+H]⁺.

### Compound 618

### (1s,3S)-N1-(6-((2R,6S)-2-ethyl-6-methylmorpholino)-2-methylpyridin-3-yl)cyclobutane-1,3-diamine

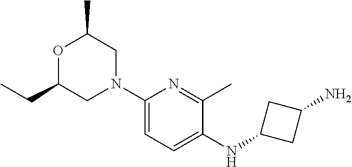 [SMILES: CC[C@@H]1CN(c2ccc(N[C@H]3C[C@@H](N)C3)c(C)n2)C[C@H](C)O1]

The titled compound 618 (1.7 mg, 4.2%) as a yellow solid was prepared according to the procedure outlined for compound 24. ¹H NMR (400 MHZ, DMSO-d₆) § 6.62 (d, J=8.8 Hz, 1H), 6.50 (d, J=8.8 Hz, 1H), 3.82 (t, J=12.8 Hz, 3H), 3.59 (d, J=10.0 Hz, 1H), 3.44 (d, J=7.6 Hz, 2H), 2.26-2.20 (m, 3H), 2.16 (t, J=11.2 Hz, 2H), 2.10-2.02 (m, 2H), 2.01-1.90 (m, 2H), 1.47 (q, J=7.6 Hz, 2H), 1.14 (d, J=6.0 Hz, 3H), 0.98-0.90 (m, 3H). Mass (m/z): 305.3 [M+H]⁺.

### Compound 619

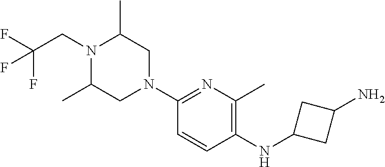 [SMILES: Cc1nc(N2CC(C)N(CC(F)(F)F)C(C)C2)ccc1NC1CC(N)C1]

The titled compound 619 (35.6 mg, 56.2%) as a light-yellow solid was prepared according to the procedure outlined for compound 24. ¹H NMR (400 MHZ, DMSO-d₆) δ 8.48-8.15 (m, 3H), 6.73-6.49 (m, 2H), 4.96-4.65 (m, 1H), 4.12-3.99 (m, 1H), 3.89-3.67 (m, 3H), 2.82-2.70 (m, 2H), 2.46-2.40 (m, 2H), 2.37-2.17 (m, 7H), 1.09 (d, J=6.2 Hz, 6H). Mass (m/z): 372.2 [M+H]⁺.

### Compound 620

### 1-(6-((6-aminospiro[3.3]heptan-2-yl)amino)naphthalen-2-yl)-4-(trifluoromethyl)piperidin-4-ol

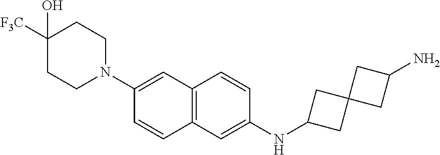 [SMILES: NC1CC2(C1)CC(Nc1ccc3cc(N4CCC(O)(C(F)(F)F)CC4)ccc3c1)C2]

The titled compound 620 (3 mg, 4.03%) as a white solid was prepared according to the procedure outlined for compound 24. ¹H NMR (400 MHZ, CD₃OD) δ 7.47 (dd, J=8.8, 5.6 Hz, 2H), 7.17 (dd, J=9.2, 2.4 Hz, 1H), 7.11 (d, J=1.6 Hz, 1H), 6.85 (dd, J=8.8, 2.4 Hz, 1H), 6.64 (d, J=1.6 Hz, 1H), 3.84 (p, J=7.6 Hz, 1H), 3.53 (d, J=12.0 Hz, 2H), 2.99 (dd, J=12.0, 10.4 Hz, 2H), 2.59-2.52 (m, 1H), 2.48-2.37 (m, 2H), 2.26-2.19 (m, 1H), 2.03-1.75 (m, 9H). Mass (m/z): 420.2 [M+H]⁺.

### Compound 621

### N-((4-(aminomethyl)cuban-1-yl)methyl)-6-((2S,6R)-2,6-dimethylmorpholino)-2-methylpyridin-3-amine

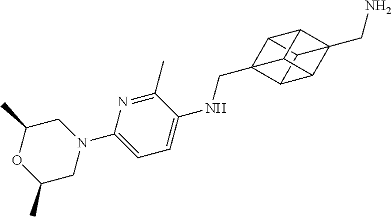 [SMILES: Cc1nc(N2C[C@@H](C)O[C@@H](C)C2)ccc1NCC12C3C4C1C1C2C3C41CN]

The titled compound 621 (11 mg, 12.5%) as a white solid was prepared according to the procedure outlined for compound 24. ¹H NMR (400 MHZ, DMSO-d₆) δ 8.49 (s, 1H), 6.97 (d, J=8.8, 1H), 6.51 (d, J=8.8, 1H), 3.85 (d, J=10.8, 2H), 3.63-3.59 (m, 8H), 3.20 (s, 2H), 3.12 (s, 2H), 2.22 (s, 2H), 2.20-2.11 (m, 2H), 1.64 (s, 1H), 1.13 (d, J=6.4, 6H). Mass (m/z): 367.3 [M+H]⁺.

### Compound 622

### N⁴-(((1r,4r)-4-aminocyclohexyl)methyl)-N′-(tert-butyl)-2-methylbenzene-1,4-diamine

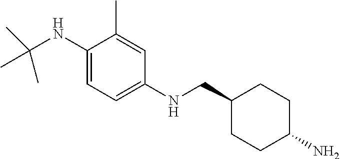 [SMILES: Cc1cc(NC[C@H]2CC[C@H](N)CC2)ccc1NC(C)(C)C]

The titled compound 622 (43 mg, 32.15%) as a white solid was prepared according to the procedure outlined for compound 24. ¹H NMR (400 MHZ, CD₃OD) δ 6.86 (d, J=8.4 Hz, 1H), 6.52-6.44 (m, 2H), 2.90 (d, J=6.8 Hz, 2H), 2.59 (m, J=10.8, 3.6 Hz, 1H), 2.21 (s, 3H), 1.91 (d, J=9.6 Hz, 4H), 1.59-1.51 (m, 1H), 1.21 (s, 9H), 1.17-1.01 (m, 4H). Mass (m/z): 290.0 [M+H]⁺.

### Compound 623

### N²-(2-methyl-6-(4-oxa-7-azaspiro[2.5]octan-7-yl)pyridin-3-yl)spiro[3.3]heptane-2,6-diamine

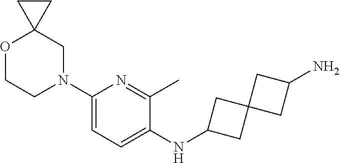 [SMILES: Cc1nc(N2CCOC3(CC3)C2)ccc1NC1CC2(CC(N)C2)C1]

The titled compound 623 (15.3 mg, 12.10%) as a white solid was prepared according to the procedure outlined for compound 24. ¹H NMR (400 MHZ, CD₃OD) δ 6.89 (d, J=8.8 Hz, 1H), 6.55 (d, J=8.8 Hz, 1H), 3.90-3.85 (m, 2H), 3.73 (t, J=7.6 Hz, 1H), 3.37 (s, 1H), 3.31 (dd, J=3.2, 1.6 Hz, 2H), 3.23 (s, 2H), 2.56-2.35 (m, 3H), 2.29 (s, 3H), 2.28-2.23 (m, 1H), 1.96-1.77 (m, 4H), 0.79 (d, J=5.2 Hz, 2H), 0.72-0.64 (m, 2H). Mass (m/z): 328.9 [M+H]⁺.

### Compound 624

### N⁷-(6-(2,6-dimethylmorpholino)-2-methylpyridin-3-yl)spiro[3.5]nonane-2,7-diamine

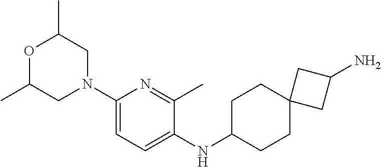 [SMILES: Cc1nc(N2CC(C)OC(C)C2)ccc1NC1CCC2(CC1)CC(N)C2]

The titled compound 624 (57.4 mg, 50.8%) as a yellow solid was prepared according to the procedure outlined for compound 24. ¹H NMR (400 MHZ, DMSO-d₆) δ 8.48 (s, 3H), 7.02 (s, 1H), 6.56 (s, 1H), 4.03-3.81 (m, 2H), 3.60 (t, J=8.4 Hz, 3H), 3.06 (s, 1H), 2.39-2.08 (m, 6H), 1.98 (d, J=8.0 Hz, 2H), 1.89 (t, J=10.0 Hz, 1H), 1.74 (t, J=14.4 Hz, 4H), 1.46-1.26 (m, 3H), 1.14 (d, J=6.4 Hz, 6H). Mass (m/z): 359.2 [M+H]⁺.

### Compound 625

### 6-((6-(4,4-dimethyl-1,4-azasilinan-1-yl)-2-methylpyridin-3-yl)amino)spiro[3.3]heptan-2-ol

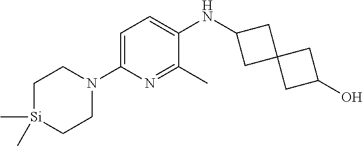 [SMILES: Cc1nc(N2CC[Si](C)(C)CC2)ccc1NC1CC2(CC(O)C2)C1]

The title compound 625 (24.7 mg, 27%) as a yellow oil was prepared according to the procedure outlined for compound 5. ¹H NMR (400 MHZ, CD₃OD) δ 6.88 (d, J=8.8 Hz, 1H), 6.52 (d, J=8.8 Hz, 1H), 4.16-4.05 (m, 1H), 3.78-3.65 (m, 5H), 2.51-2.42 (m, 2H), 2.40-2.33 (m, 1H), 2.31-2.24 (m, 4H), 2.00-1.88 (m, 4H), 0.78-0.73 (m, 4H), 0.07 (s, 6H). Mass (m/z): 346.2 [M+H]⁺.

### Compound 626

### N²-(2-methyl-6-(5-oxa-8-azaspiro[3.5]nonan-8-yl)pyridin-3-yl)spiro[3.3]heptane-2,6-diamine

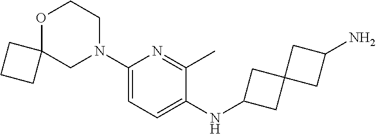 [SMILES: Cc1nc(N2CCOC3(CCC3)C2)ccc1NC1CC2(CC(N)C2)C1]

The titled compound 626 (4.0 mg, 6.2%) as a white solid was prepared according to the procedure outlined for compound 24. ¹HNMR (400 MHZ, CD₃OD) δ 6.78 (d, J=8.8 Hz, 1H), 6.46 (d, J=8.8 Hz, 1H), 3.64-3.60 (m, 3H), 3.22-3.19 (m, 1H), 3.07-3.04 (m, 2H), 2.44-2.39 (m, 1H), 2.35 (dd, J=11.2, 5.2 Hz, 1H), 2.29-2.24 (m, 1H), 2.19 (s, 3H), 2.15 (dd, J=8.8, 3.6 Hz, 1H), 2.04-1.88 (m, 5H), 1.85-1.61 (m, 7H). Mass (m/z): 343.3 [M+H]⁺

### Compound 627

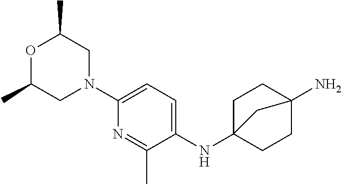 [SMILES: Cc1nc(N2C[C@@H](C)O[C@@H](C)C2)ccc1NC12CCC(N)(CC1)C2]

The titled compound 627 (21.1 mg, 32.2%) as a light-yellow solid was prepared according to the procedure outlined for compound 24. ¹H NMR (400 MHZ, DMSO-d₆) δ 8.38 (br s, 3H), 6.74 (d, J=8.4 Hz, 1H), 6.49 (d, J=8.4 Hz, 1H), 4.75-4.42 (m, 1H), 3.83 (d, J=12.0 Hz, 2H), 3.72-3.41 (m, 5H), 2.49-2.43 (m, 1H), 2.42-2.28 (m, 2H), 2.23 (s, 3H), 2.20-2.06 (m, 5H), 1.98-1.84 (m, 2H), 1.12 (d, J=6.2 Hz, 6H). Mass (m/z): 331.3 [M+H]⁺.

### Compound 628

### N²-(2-methyl-4-(2,2,6,6-tetramethylmorpholino)phenyl)spiro[3.3]heptane-2,6-diamine

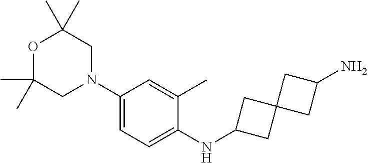 [SMILES: Cc1cc(N2CC(C)(C)OC(C)(C)C2)ccc1NC1CC2(CC(N)C2)C1]

The titled compound 628 (24.6 mg, 36.5%) as a yellow solid was prepared according to the procedure outlined for compound 24. ¹H NMR (400 MHZ, DMSO-d₆) δ 8.39 (s, 3H), 6.73-6.52 (m, 2H), 6.34 (s, 1H), 4.59 (s, 1H), 3.68 (s, 1H), 3.58-3.47 (m, 1H), 2.75-2.62 (m, 3H), 2.42-2.31 (m, 2H), 2.28-2.15 (m, 3H), 2.13-2.01 (m, 3H), 1.99-1.86 (m, 2H), 1.20 (s, 12H). Mass (m/z): 358.3 [M+H]⁺.

### Compound 629

### N¹-(6-(2-methylmorpholino) naphthalen-2-yl)cyclobutane-1,3-diamine

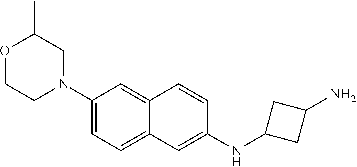 [SMILES: CC1CN(c2ccc3cc(NC4CC(N)C4)ccc3c2)CCO1]

The titled compound 629 (1.2 mg, 1.09%) as a white solid was prepared according to the procedure outlined for compound 24. ¹H NMR (400 MHZ, DMSO-d₆) δ 7.46 (d, J=8.8 Hz, 2H), 7.17 (dd, J=9.2, 2.4 Hz, 1H), 6.98 (d, J=2.4 Hz, 1H), 6.86 (dd, J=8.8, 2.4 Hz, 1H), 6.43 (d, J=2.0 Hz, 1H), 5.87 (d, J=5.6 Hz, 1H), 3.91 (dd, J=11.2, 1.6 Hz, 2H), 3.71-3.63 (m, 2H), 3.57-3.44 (m, 3H), 2.65-2.60 (m, 1H), 2.31 (dd, J=11.6, 10.4 Hz, 1H), 2.11-2.01 (m, 4H), 1.16 (d, J=6.4 Hz, 3H). Mass (m/z): 311.9 [M+H]⁺.

### Compound 630

### N¹-(6-(2-methylmorpholino)naphthalen-2-yl)cyclobutane-1,3-diamine

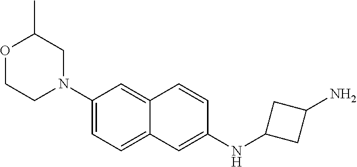 [SMILES: CC1CN(c2ccc3cc(NC4CC(N)C4)ccc3c2)CCO1]

The titled compound 630 (20.3 mg, 18.37%) as a white solid was prepared according to the procedure outlined for compound 24. ¹H NMR (400 MHZ, DMSO-d₆) δ 7.45 (dd, J=8.8, 4.4 Hz, 2H), 7.17 (dd, J=8.8, 2.4 Hz, 1H), 6.98 (d, J=2.4 Hz, 1H), 6.85 (dd, J=8.8, 2.2 Hz, 1H), 6.53 (d, J=2.0 Hz, 1H), 5.77 (d, J=6.4 Hz, 1H), 3.91 (dd, J=11.2, 2.0 Hz, 1H), 3.71-3.63 (m, 2H), 3.55 (d, J=11.6 Hz, 1H), 3.46 (d, J=11.6 Hz, 1H), 3.39 (d, J=6.8 Hz, 1H), 3.11-3.03 (m, 1H), 2.74-2.66 (m, 2H), 2.62 (td, J=11.6, 3.2 Hz, 1H), 2.31 (dd, J=11.6, 10.0 Hz, 1H), 1.49 (J=8.8, 2.8 Hz, 2H), 1.16 (d, J=6.2 Hz, 3H). Mass (m/z): 311.9 [M+H]⁺.

### Compound 631

### N1-(6-((2R,6S)-2,6-dimethylmorpholino)-2-methylpyridin-3-yl)cyclooctane-1,5-diamine

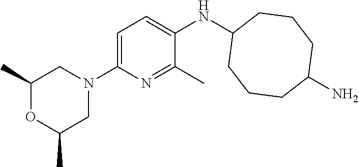 [SMILES: Cc1nc(N2C[C@@H](C)O[C@@H](C)C2)ccc1NC1CCCC(N)CCC1]

The title compound 631 (8.1 mg, 13%) as a yellow oil was prepared according to the procedure outlined for compound 24. ¹HNMR (400 MHZ, CD₃OD) δ 7.57 (d, J=9.4 Hz, 1H), 7.07 (d, J=9.5 Hz, 1H), 3.75 (d, J=10.8 Hz, 4H), 3.59 (d, J=2.8 Hz, 1H), 3.38 (t, J=9.2 Hz, 1H), 2.64 (dd, J=16.2, 7.2 Hz, 2H), 2.47 (s, 3H), 1.81 (m, 12H), 1.21 (d, J=6.2 Hz, 6H). Mass (m/z): 346.9 [M+H]⁺.

### Compound 632

### (1s,4s)-4-((6-(2,6-dimethylmorpholino) naphthalen-2-yl)amino)cyclohexane-1-carboxamide

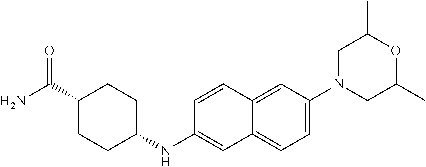 [SMILES: CC1CN(c2ccc3cc(N[C@H]4CC[C@@H](C(N)=O)CC4)ccc3c2)CC(C)O1]

The titled compound 632 (12.7 mg, 19.0%) as a purple solid was prepared according to the procedure outlined for compound 24. ¹H NMR (400 MHZ, DMSO-d₆) δ 7.44 (d, J=8.8 Hz, 2H), 7.20-7.14 (m, 2H), 7.00-6.94 (m, 2H), 6.71 (s, 1H), 6.63 (d, J=2.0 Hz, 1H), 5.49 (d, J=7.2 Hz, 1H), 3.78-3.68 (m, 2H), 3.58-3.49 (m, 3H), 2.27-2.15 (m, 3H), 1.89-1.69 (m, 4H), 1.67-1.48 (m, 4H), 1.16 (d, J=6.0 Hz, 6H). Mass (m/z): 382.3 [M+H]⁺.

### Compound 633

### 4-((6-(4-(trifluoromethyl)piperidin-1-yl) naphthalen-2-yl)amino)piperidine-1-carboxamide

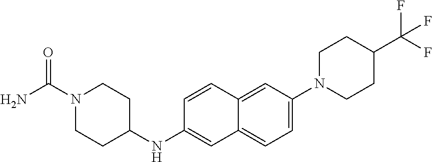 [SMILES: NC(=O)N1CCC(Nc2ccc3cc(N4CCC(C(F)(F)F)CC4)ccc3c2)CC1]

The titled compound 633 (2.8 mg, 4.9%) as a orang solid was prepared according to the procedure outlined for compound 24. ¹H NMR (400 MHZ, DMSO-d₆) δ 7.46 (d, J=8.8 Hz, 2H), 7.17 (dd, J=8.8, 2.4 Hz, 1H), 7.02 (d, J=2.4 Hz, 1H), 6.89 (dd, J=8.8, 2.4 Hz, 1H), 6.71 (d, J=2.4 Hz, 1H), 5.92 (s, 2H), 5.51 (d, J=8.0 Hz, 1H), 3.88 (d, J=13.6 Hz, 2H), 3.74 (d, J=12.0 Hz, 2H), 3.54-3.42 (m, 1H), 2.96-2.83 (m, 2H), 2.75-2.62 (m, 3H), 1.97-1.84 (m, 4H), 1.67-1.53 (m, 2H), 1.32-1.24 (m, 2H). Mass (m/z): 421.2 [M+H]⁺.

### Compound 634

### N2-(4-(4.4-dimethyl-1,4-azasilinan-1-yl)-2-methylphenyl)spiro[3.3]heptane-2,6-diamine

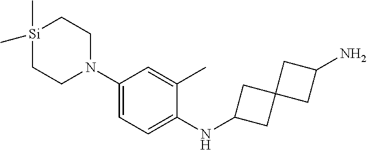 [SMILES: Cc1cc(N2CC[Si](C)(C)CC2)ccc1NC1CC2(CC(N)C2)C1]

The titled compound 634 (32.4 mg, 38.9%) as a white solid was prepared according to the procedure outlined for compound 24. ¹H NMR (400 MHZ, CDCl₃) δ 6.63 (d, J=8.8 Hz, 2H), 6.36 (d, J=8.1 Hz, 1H), 3.74-3.68 (m, 1H), 3.41 (d, J=5.6 Hz, 4H), 2.49-2.22 (m, 7H), 2.04 (s, 3H), 1.74 (ddd, J=22.2, 10.8, 5.3 Hz, 5H), 0.77-0.68 (m, 6H). Mass (m/z): 344.2 [M+H]⁺.

### Compound 635

### N1-(6-(3,5-dimethyl-4-(2,2,2-trifluoroethyl) piperazin-1-yl)-2-ethyl-5-fluoropyridin-3-yl)cyclobutane-1,3-diamine

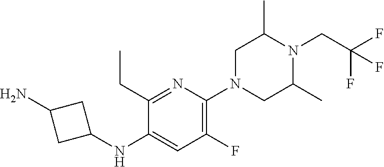 [SMILES: CCc1nc(N2CC(C)N(CC(F)(F)F)C(C)C2)c(F)cc1NC1CC(N)C1]

The titled compound 635 (26.2, 50%) as an oil was prepared according to the procedure outlined for compound 24. ¹H NMR (400 MHZ, CD₃OD) δ 6.60 (dd, J=47.5, 14.1 Hz, 1H), 3.97-3.87 (m, 0.5H), 3.65 (dd, J=13.8, 6.3 Hz, 0.5H), 3.50 (d, J=12.2 Hz, 2H), 3.37 (s, 2H), 3.19 (dd, J=15.3, 7.9 Hz, 1H), 2.95 (s, 2H), 2.84-2.72 (m, 1H), 2.59 (s, 4H), 2.30-2.14 (m, 2H), 1.65 (ddd, J=17.4, 8.7, 2.8 Hz, 1H), 1.23 (td, J=7.5, 2.0 Hz, 3H), 1.14 (d, J=6.3 Hz, 6H). Mass (m/z): 404.3 [M+H]⁺.

### Compound 636

### N2-(4-(3,5-dimethyl-4-(2,2,2-trifluoroethyl) piperazin-1-yl)-2-methylphenyl)spiro[3.3]heptane-2,6-diamine

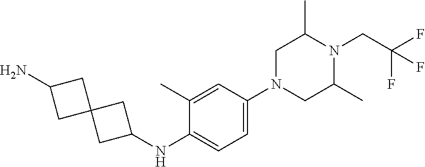 [SMILES: Cc1cc(N2CC(C)N(CC(F)(F)F)C(C)C2)ccc1NC1CC2(CC(N)C2)C1]

The titled compound 636 (8 mg, 10%) as an oil was prepared according to the procedure outlined for compound 24. ¹H NMR (400 MHZ, CD₃OD) δ 6.73 (m, 2H), 6.64 (m, 3H), 6.47 (m, 1H), 3.77 (m, 1H), 3.37 (m, 3H), 3.25 (d, J=11.4 Hz, 2H), 2.93 (m, 2H), 2.55 (td, J=11.4, 5.8 Hz, 1H), 2.47 (m, 1H), 2.37 (dt, J=11.4, 9.2 Hz, 3H), 2.27 (m, 1H), 2.12 (s, 3H), 1.89 (m, 4H), 1.16 (d, J=6.3 Hz, 7H). Mass (m/z): 411.3 [M+H]⁺.

### Compound 637

### N²-(2-(difluoromethoxy)-6-((25,6R)-2,6-dimethylmorpholino)pyridin-3-yl)spiro[3.3]heptane-2, 6-diamine

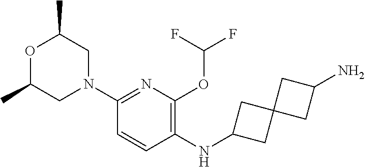 [SMILES: C[C@@H]1CN(c2ccc(NC3CC4(CC(N)C4)C3)c(OC(F)F)n2)C[C@H](C)O1]

The titled compound 637 (18.6 mg, 48.9%) as a purple solid was prepared according to the procedure outlined for compound 24. ¹H NMR (400 MHZ, DMSO-d₆) δ 8.18 (s, 2H), 7.70 (t, J=73.5 Hz, 2H), 7.00-6.88 (m, 1H), 6.47 (d, J=8.4 Hz, 1H), 4.68 (s, 1H), 3.89-3.77 (m, 2H), 3.73-3.52 (m, 4H), 2.49-2.43 (m, 2H), 2.40-2.28 (m, 2H), 2.24-2.13 (m, 4H), 2.00-1.86 (m, 2H). Mass (m/z): 383.2 [M+H]⁺.

### Compound 638

### N²-(6-(4,4-dimethyl-1,4-azasilinan-1-yl)-5-fluoro-2-methylpyridin-3-yl)spiro[3.3]heptane-2,6-diamine

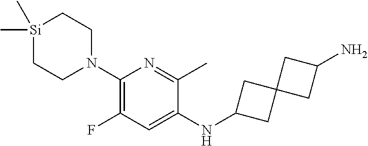 [SMILES: Cc1nc(N2CC[Si](C)(C)CC2)c(F)cc1NC1CC2(CC(N)C2)C1]

The titled compound 638 (59.1 mg, 75.42%) as a white solid was prepared according to the procedure outlined for compound 24. ¹HNMR (400 MHZ, CD₃OD) δ 6.61 (d, J=14.0 Hz, 1H), 3.74-3.66 (m, 1H), 3.51-3.44 (m, 4H), 3.28 (dd, J=8.8, 7.2 Hz, 1H), 2.57-2.36 (m, 3H), 2.31-2.24 (m, 1H), 2.24 (d, J=1.2 Hz, 3H), 1.97-1.77 (m, 4H), 0.90-0.84 (m, 4H), 0.13-0.08 (m, 6H). Mass (m/z): 363.3 [M+H]⁺.

### Compound 639

### N⁶-(6-aminospiro[3.3]heptan-2-yl)-N²-(2,2,2-trifluoroethyl) quinoline-2,6-diamine

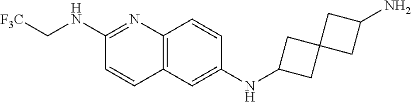 [SMILES: NC1CC2(C1)CC(Nc1ccc3nc(NCC(F)(F)F)ccc3c1)C2]

The titled compound 639 (10.3 mg, 13.24%) as a white solid was prepared according to the procedure outlined for compound 24. ¹HNMR (400 MHZ, CD₃OD) δ 7.75 (d, J=8.8 Hz, 1H), 7.47 (d, J=9.2 Hz, 1H), 7.01 (dd, J=9.2, 2.4 Hz, 1H), 6.74 (d, J=8.8 Hz, 1H), 6.64 (d, J=2.4 Hz, 1H), 4.23 (q, J=9.6 Hz, 2H), 3.90-3.82 (m, 1H), 3.36 (dd, J=14.4, 7.2 Hz, 1H), 2.63-2.56 (m, 1H), 2.53-2. 41 (m, 2H), 2.32-2.24 (m, 1H), 1.99-1.79 (m, 4H). Mass (m/z): 351.2 [M+H]⁺.

### Compound 640

### N2-(4-((25,6R)-2,6-dimethylmorpholino)-2-ethylphenyl)spiro[3.3]heptane-2,6-diamine

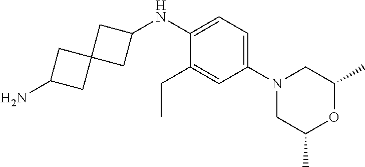 [SMILES: CCc1cc(N2C[C@@H](C)O[C@@H](C)C2)ccc1NC1CC2(CC(N)C2)C1]

The title compound 640 (41.6 mg, 73%) as a yellow solid was prepared according to the procedure outlined for compound 24. ¹H NMR (400 MHZ, CD₃OD_SPE) δ 6.76 (d, J=2.4 Hz, 1H), 6.72 (d, J=8.6 Hz, 1H), 6.50 (d, J=8.6 Hz, 1H), 3.85-3.73 (m, 3H), 3.42-3.33 (m, 1H), 3.27 (d, J=10.9 Hz, 2H), 2.59-2.43 (m, 4H), 2.42-2.36 (m, 1H), 2.31-2.22 (m, 3H), 1.99-1.82 (m, 4H), 1.22-1.16 (m, 9H). Mass (m/z): 344.2 [M+H]⁺.

### Compound 641

### N²-(6-(3,5-dimethyl-4-(2, 2,2-trifluoroethyl)piperazin-1-yl)-5-fluoro-2-methylpyridin-3-yl)spiro [3.3]heptane-2,6-diamine

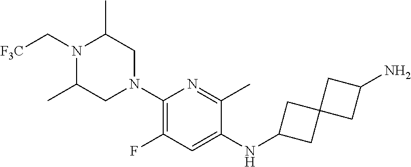 [SMILES: Cc1nc(N2CC(C)N(CC(F)(F)F)C(C)C2)c(F)cc1NC1CC2(CC(N)C2)C1]

The titled compound 641 (5 mg, 31.2%) as a white solid was prepared according to the procedure outlined for compound 24. ¹H NMR (400 MHZ, CD₃OD) δ 6.59 (d, J=13.6 Hz, 1H), 3.74-3.63 (m, 1H), 3.47-3.30 (m, 5H), 2.90 (d, J=6.4 Hz, 2H), 2.60-2.35 (m, 5H), 2.32-2.18 (m, 4H), 1.93-1.90 (m, 4H), 1.11 (d, J=6.4 Hz, 6H). Mass (m/z): 430.1 [M+H]⁺.

### Compound 642

### 1-(4-(5-((8-azabicyclo[3.2.1]octan-3-yl)amino)-6-methylpyridin-2-yl)-2,6-dimethylpiperazin-1-yl)-2,2,2-trifluoroethan-1-one

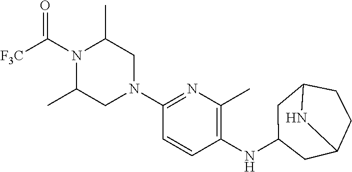 [SMILES: Cc1nc(N2CC(C)N(C(=O)C(F)(F)F)C(C)C2)ccc1NC1CC2CCC(C1)N2]

The titled compound 642 (27.9 mg, 28.76%) as a white solid was prepared according to the procedure outlined for compound 24. ¹H NMR (400 MHZ, CDCl₃) δ 6.78 (d, J=8.8 Hz, 1H), 6.51 (d, J=8.8 Hz, 1H), 4.67 (s, 1H), 4.26 (s, 1H), 3.97 (s, 2H), 3.68 (d, J=4.4 Hz, 3H), 3.28 (s, 1H), 2.90 (s, 2H), 2.33 (s, 3H), 2.29-2.23 (m, 2H), 2.11 (s, 1H), 2.01-1.95 (m, 2H), 1.81 (d, J=14.4 Hz, 2H), 1.46 (d, J=17.2 Hz, 6H). Mass (m/z): 426.2 [M+H]⁺.

### Compound 643

### N-(6-(3,5-dimethyl-4-(2,2,2-trifluoroethyl) piperazin-1-yl)-2-methylpyridin-3-yl)-8-azabicyclo[3.2.1]octan-3-amine

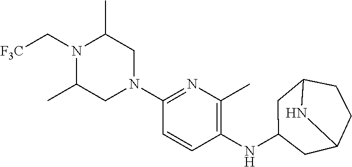 [SMILES: Cc1nc(N2CC(C)N(CC(F)(F)F)C(C)C2)ccc1NC1CC2CCC(C1)N2]

The titled compound 643 (4.5 mg, 4.65%) as a white solid was prepared according to the procedure outlined for compound 24. ¹H NMR (400 MHz, CDCl₃) δ 6.75 (d, J=8.8 Hz, 1H), 6.44 (d, J=8.8 Hz, 1H), 3.85 (d, J=11.2 Hz, 2H), 3.65 (s, 3H), 3.33-3.26 (m, 2H), 2.97 (s, 2H), 2.48-2.42 (m, 2H), 2.32 (s, 3H), 2.21 (d, J=11.2 Hz, 2H), 2.09 (d, J=7.6 Hz, 2H), 1.95 (s, 2H), 1.79 (d, J=14.4 Hz, 2H), 1.19 (d, J=6.4 Hz, 6H). Mass (m/z): 412.1 [M+H]⁺.

### Compound 644

### 1-(4-(4-((6-aminospiro[3.3]heptan-2-yl)amino)-3-methylphenyl)-2,6-dimethylpiperazin-1-yl)-2, 2,2-trifluoroethan-1-one

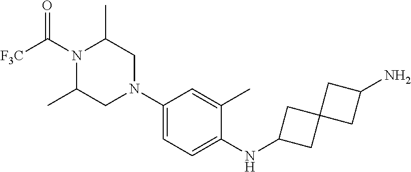 [SMILES: Cc1cc(N2CC(C)N(C(=O)C(F)(F)F)C(C)C2)ccc1NC1CC2(CC(N)C2)C1]

The titled compound 644 (12.5 mg, 10.30%) as a white solid was prepared according to the procedure outlined for compound 24. ¹H NMR (400 MHZ, CDCl₃) δ 6.75-6.70 (m, 2H), 6.43 (d, J=8.4 Hz, 1H), 4.63 (s, 1H), 4.21 (s, 1H), 3.84-3.76 (m, 1H), 3.42 (s, 1H), 3.21 (d, J=11.2 Hz, 2H), 2.84 (d, J=12.0 Hz, 2H), 2.58-2.43 (m, 3H), 2.30 (dd, J=11.2, 5.6 Hz, 1H), 2.12 (s, 3H), 1.89-1.81 (m, 4H), 1.51 (d, J=24.4 Hz, 6H). Mass (m/z): 425.1 [M+H]⁺.

### Compound 645

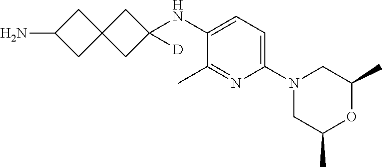 [SMILES: [2H]C1(Nc2ccc(N3C[C@@H](C)O[C@@H](C)C3)nc2C)CC2(CC(N)C2)C1]

The titled compound 645 (15.2 mg, 21.1%) as a light-yellow solid was prepared according to the procedure outlined for compound 24. ¹H NMR (400 MHZ, DMSO-d₆) δ 8.11 (br s, 2H), 6.81-6.67 (m, 1H), 6.50 (d, J=8.4 Hz, 1H), 4.54 (br s, 1H), 3.83 (d, J=12.0 Hz, 2H), 3.68-3.52 (m, 3H), 2.48-2.27 (m, 4H), 2.21 (s, 3H), 2.19-2.07 (m, 4H), 1.96-1.82 (m, 2H), 1.13 (d, J=6.2 Hz, 6H). Mass (m/z): 332.3 [M+H]⁺.

### Compound 646

### N2-(2-(2,2,6,6-tetramethylmorpholino)quinolin-6-yl)spiro[3.3]heptane-2,6-diamine

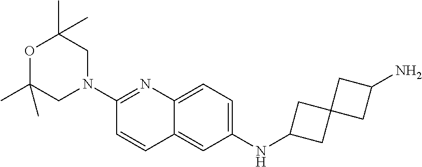 [SMILES: CC1(C)CN(c2ccc3cc(NC4CC5(CC(N)C5)C4)ccc3n2)CC(C)(C)O1]

The titled compound 646 (10 mg, 41.6%) as a white solid was prepared according to the procedure outlined for compound 24. ¹H NMR (400 MHz, CD₃OD) δ 7.79 (d, J=9.2 Hz, 1H), 7.44 (d, J=9.2 Hz, 1H), 6.99 (dd, J=9.2, 2.4 Hz, 2H), 6.59 (d, J=2.4 Hz, 1H), 3.89-3.75 (m, 1H), 3.47 (s, 4H), 2.61-2.35 (m, 3H), 2.25-2.23 (m, 1H), 1.95-1.74 (m, 4H), 1.26 (s, 12H). Mass (m/z): 395.1 [M+H]⁺.

### Compound 647

### 1-(4-(4-((6-aminospiro[3.3]heptan-2-yl)amino)-2-methylphenyl)-2,6-dimethylpiperazin-1-yl)-2, 2,2-trifluoroethan-1-one

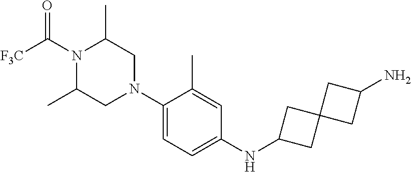 [SMILES: Cc1cc(NC2CC3(CC(N)C3)C2)ccc1N1CC(C)N(C(=O)C(F)(F)F)C(C)C1]

The titled compound 647 (15.4 mg, 23.79%) as a white solid was prepared according to the procedure outlined for compound 24. ¹H NMR (400 MHZ, CDCl₃) δ 6.86 (d, J=8.4 Hz, 1H), 6.39 (d, J=2.4 Hz, 1H), 6.35 (dd, J=8.4, 2.8 Hz, 1H), 4.62 (s, 1H), 4.20 (s, 1H), 3.80-3.73 (m, 1H), 3.46-3.39 (m, 1H), 2.87 (s, 4H), 2.55-2.41 (m, 3H), 2.32 (d, J=12.0 Hz, 4H), 1.85 (m, J=11.6, 8.8 Hz, 4H), 1.60-1.51 (m, 6H). Mass (m/z): 429.2 [M+H]⁺.

### Compound 648

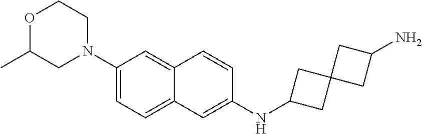 [SMILES: CC1CN(c2ccc3cc(NC4CC5(CC(N)C5)C4)ccc3c2)CCO1]

The titled compound 648 (48.8 mg, 83.1%) as a white solid was prepared according to the procedure outlined for compound 24. ¹H NMR (400 MHZ, DMSO-d₆) δ 7.97 (s, 2H), 7.46 (dd, J=9.0, 2.2 Hz, 2H), 7.18 (dd, J=9.0, 2.5 Hz, 1H), 6.98 (d, J=2.5 Hz, 1H), 6.83 (dd, J=8.8, 2.3 Hz, 1H), 6.52 (d, J=2.3 Hz, 1H), 5.85 (d, J=6.4 Hz, 1H), 3.95-3.89 (m, 1H), 3.82-3.74 (m, 1H), 3.71-3.63 (m, 2H), 3.61-3.52 (m, 2H), 3.50-3.43 (m, 1H), 2.70-2.55 (m, 2H), 2.47-2.40 (m, 2H), 2.37-2.28 (m, 1H), 2.26-2.10 (m, 3H), 1.95-1.82 (m, 2H), 1.16 (d, J=6.2 Hz, 3H). Mass (m/z): 352.4 [M+H]⁺.

Compound 649

### N-(3-(aminomethyl)cyclobutyl)-6-((2S,6R)-2,6-dimethylmorpholino) naphthalen-2-amine

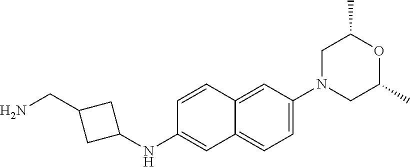 [SMILES: C[C@@H]1CN(c2ccc3cc(NC4CC(CN)C4)ccc3c2)C[C@H](C)O1]

The titled compound 649 (7.6 mg, 10.2%) as a yellow solid was prepared according to the procedure outlined for compound 24. ¹H NMR (400 MHZ, DMSO-d₆) δ 7.45 (d, J=8.8 Hz, 2H), 7.18 (dd, J=9.2, 2.4 Hz, 1H), 6.97 (d, J=2.4 Hz, 1H), 6.88-6.82 (m, 1H), 6.56-6.44 (m, 1H), 5.82 (d, J=6.4 Hz, 1H), 3.91 (h, J=6.8 Hz, 1H), 3.78-3.66 (m, 2H), 3.59-3.51 (m, 2H), 3.30 (s, 1H), 2.24 (t, J=10.4 Hz, 2H), 2.11-2.19 (m, 2H), 1.96-1.85 (m, 1H), 1.29 (s, 1H), 1.16 (d, J=6.0 Hz, 6H). Mass (m/z): 340.2 [M+H]⁺.

### Compound 650

### N2-(6-aminospiro [3.3]heptan-2-yl)-N6-(2-methoxyethyl) naphthalene-2,6-diamine

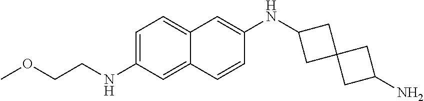 [SMILES: COCCNc1ccc2cc(NC3CC4(CC(N)C4)C3)ccc2c1]

The titled compound 650 (18.4 mg, 24.6%) as a light-yellow solid was prepared according to the procedure outlined for compound 24. ¹H NMR (400 MHZ, DMSO-d₆) δ 8.18 (br s, 2H), 7.49-7.12 (m, 3H), 6.88 (d, J=8.8 Hz, 1H), 6.79 (d, J=8.8 Hz, 1H), 6.63 (s, 1H), 6.51 (s, 1H), 5.69 (br s, 1H), 3.76 (s, 1H), 3.66-3.48 (m, 3H), 3.29 (s, 3H), 3.26-3.16 (m, 2H), 2.63-2.53 (m, 1H), 2.46-2.34 (m, 2H), 2.27-2.10 (m, 3H), 1.98-1.79 (m, 2H). Mass (m/z): 326.3 [M+H]⁺.

### Compound 651

### N²-(6-aminospiro [3.3]heptan-2-yl)-N⁶-propylnaphthalene-2,6-diamine

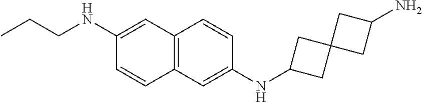 [SMILES: CCCNc1ccc2cc(NC3CC4(CC(N)C4)C3)ccc2c1]

The titled compound 651 (13.5 mg, 8.81%) as a yellow solid was prepared according to the procedure outlined for compound 24. ¹H NMR (400 MHZ, CD₃OD) δ 7.41 (d, J=7.6 Hz, 2H), 6.90 (dd, J=8.8, 2.4 Hz, 1H), 6.85 (dd, J=8.8, 2.4 Hz, 1H), 6.77 (d, J=1.6 Hz, 1H), 6.69 (d, J=1.6 Hz, 1H), 3.90-3.82 (m, 1H), 3.34 (d, J=4.4 Hz, 1H), 3.11 (t, J=7.2 Hz, 2H), 2.62-2.55 (m, 1H), 2.52-2.40 (m, 2H), 2.31-2.24 (m, 1H), 1.96-1.81 (m, 4H), 1.70 (dd, J=14.4, 7.2 Hz, 2H), 1.05 (t, J=7.2 Hz, 3H). Mass (m/z): 310.1 [M+H]⁺.

### Compound 652

### N²-(2-(2, 2-dimethylmorpholino) quinazolin-6-yl)spiro[3.3]heptane-2,6-diamine

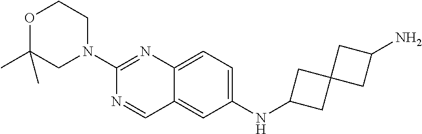 [SMILES: CC1(C)CN(c2ncc3cc(NC4CC5(CC(N)C5)C4)ccc3n2)CCO1]

The titled compound 652 (4.9 mg, 4.10%) as a yellow solid was prepared according to the procedure outlined for compound 24. ¹H NMR (400 MHZ, DMSO-d₆) δ 8.96 (s, 1H), 7.31 (d, J=9.2 Hz, 1H), 7.17 (dd, J=9.2, 2.4 Hz, 1H), 6.53 (d, J=2.4 Hz, 1H), 6.05 (d, J=6.4 Hz, 1H), 3.72 (dd, J=14.4, 7.2 Hz, 1H), 3.63 (s, 4H), 3.20 (d, J=7.6 Hz, 2H), 2.39-2.33 (m, 2H), 2.13 (dd, J=10.4, 5.6 Hz, 1H), 2.04-1.87 (m, 1H), 1.81 (d, J=8.0 Hz, 3H), 1.70 (s, 2H), 1.18 (s, 12H). Mass (m/z): 396.1 [M+H]⁺.

### Compound 653

### N2-(6-(2-(trifluoromethyl)morpholino)naphthalen-2-yl)spiro[3.3]heptane-2,6-diamine

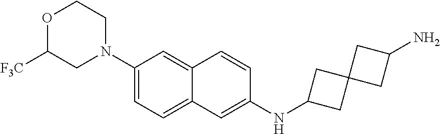 [SMILES: NC1CC2(C1)CC(Nc1ccc3cc(N4CCOC(C(F)(F)F)C4)ccc3c1)C2]

The titled compound 653 (10.6 mg, 14.58%) as a white solid was prepared according to the procedure outlined for compound 24. ¹H NMR (400 MHz, DMSO-d₆) δ 7.49 (dd, J=8.8, 5.2 Hz, 2H), 7.22 (dd, J=9.2, 2.4 Hz, 1H), 7.09 (s, 1H), 6.85 (dd, J=8.8, 2.4 Hz, 1H), 6.52 (s, 1H), 5.87 (d, J=6.4 Hz, 1H), 4.37 (s, 1H), 4.09 (d, J=9.2 Hz, 1H), 3.86-3.64 (m, 4H), 3.55 (d, J=12.0 Hz, 1H), 3.24-3.18 (m, 1H), 2.79-2.66 (m, 3H), 2.49-2.46 (m, 2H), 2.40-2.32 (m, 2H), 2.13 (dd, J=11.2, 5.2 Hz, 1H), 1.88-1.63 (m, 4H). Mass (m/z): 406.2 [M+H]⁺.

### Compound 654

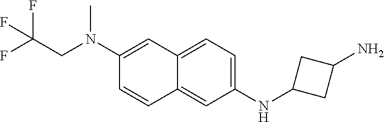 [SMILES: CN(CC(F)(F)F)c1ccc2cc(NC3CC(N)C3)ccc2c1]

The titled compound 654 (49.6 mg, 78.3%) as a white solid was prepared according to the procedure outlined for compound 24. ¹H NMR (400 MHZ, DMSO-d₆) δ 8.25 (s, 2H), 7.48 (d, J=8.8 Hz, 2H), 7.17 (dd, J=9.0, 2.6 Hz, 1H), 6.95 (d, J=2.7 Hz, 1H), 6.90-6.81 (m, 1H), 6.58-6.43 (m, 1H), 6.06-5.89 (m, 1H), 4.34-4.19 (m, 2H), 4.15-4.05 (m, 1H), 3.88-3.75 (m, 1H), 3.03 (s, 3H), 2.57-2.51 (m, 1H), 2.31-2.17 (m, 2H). Mass (m/z): 324.2 [M+H]⁺.

### Compound 655

### N⁶-(3-aminocyclobutyl)-N²-methyl-N²-(2,2,2-trifluoroethyl) quinoline-2,6-diamine

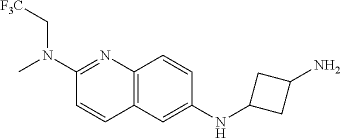 [SMILES: CN(CC(F)(F)F)c1ccc2cc(NC3CC(N)C3)ccc2n1]

The titled compound 655 (6.6 mg, 5.96%) as a yellow solid was prepared according to the procedure outlined for compound 24. ¹H NMR (400 MHZ, CD₃OD) δ 7.75 (d, J=9.2 Hz, 1H), 7.39 (d, J=9.2 Hz, 1H), 6.95-6.88 (m, 2H), 6.56 (d, J=2.8 Hz, 1H), 4.34 (q, J=9.2 Hz, 2H), 3.47 (dd, J=11.2, 4.0 Hz, 1H), 3.14-3.08 (m, 4H), 2.76-2.70 (m, 2H), 1.58-1.45 (m, 2H). Mass (m/z): 325.0 [M+H]⁺.

### Compound 656

### N⁵-(8-azabicyclo[3.2.1]octan-3-yl)-N²,6-dimethyl-N²-(2,2,2-trifluoroethyl)pyridine-2,5-diamine

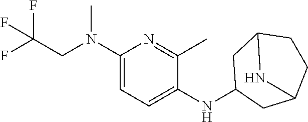 [SMILES: Cc1nc(N(C)CC(F)(F)F)ccc1NC1CC2CCC(C1)N2]

The titled compound 656 (37.2 mg, 50.6%) as a white solid was prepared according to the procedure outlined for compound 24. ¹H NMR (400 MHZ, DMSO-d₆) δ 9.40 (s, 2H), 6.88 (d, J=8.8 Hz, 1H), 6.48 (d, J=8.8 Hz, 1H), 4.45-4.27 (m, 3H), 3.87 (q, J=3.2 Hz, 2H), 3.58 (t, J=5.6 Hz, 1H), 2.98 (s, 3H), 2.27 (d, J=12.0 Hz, 7H), 2.01-1.89 (m, 4H). Mass (m/z): 329.2 [M+H]⁺.

### Compound 657

### N-(6-((2S,6R)-2,6-diethylmorpholino)-2-methylpyridin-3-yl)-8-azabicyclo[3.2.1]octan-3-amine

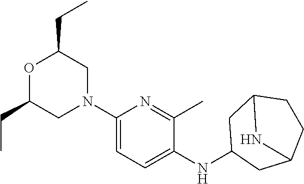 [SMILES: CC[C@@H]1CN(c2ccc(NC3CC4CCC(C3)N4)c(C)n2)C[C@H](CC)O1]

The titled compound 657 (25.9 mg, 35.7%) as a yellow solid was prepared according to the procedure outlined for compound 24. ¹H NMR (400 MHZ, DMSO-d₆) δ 9.43 (s, 1H), 9.31 (s, 1H), 6.86 (d, J=8.8 Hz, 1H), 6.57 (d, J=8.4 Hz, 1H), 4.43 (s, 1H), 3.96-3.83 (m, 4H), 3.59 (t, J=5.6 Hz, 1H), 3.96-3.83 (m, 1H), 2.33-2.16 (m, 9H), 2.00-1.88 (m, 4H), 1.56-1.40 (m, 4H), 0.95 (t, J=7.6 Hz, 6H). Mass (m/z): 359.3 [M+H]⁺.

### Compound 658

### N2-(6-aminospiro[3.3]heptan-2-yl)-N⁶-ethylnaphthalene-2,6-diamine

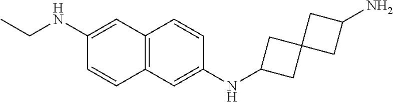 [SMILES: CCNc1ccc2cc(NC3CC4(CC(N)C4)C3)ccc2c1]

The titled compound 658 (17.1 mg, 18.66%) as a purple solid was prepared according to the procedure outlined for compound 24. ¹H NMR (400 MHz, CD₃OD) δ 7.29 (d, J=8.8 Hz, 2H), 6.82-6.73 (, 2H), 6.66 (d, J=2.0 Hz, 1H), 6.56 (d, J=2.0 Hz, 1H), 3.73 (p, J=7.6 Hz, 1H), 3.16 (dd, J=8.4, 7.6 Hz, 1H), 3.05 (q, J=7.2 Hz, 2H), 2.48-2.40 (m, 1H), 2.38-2.25 (m, 2H), 2.16-2.09 (m, 1H), 1.82-1.64 (m, 4H), 1.16 (t, J=7.2 Hz, 3H). Mass (m/z): 296.1 [M+H]⁺.

### Compound 659

### N2-(2-ethyl-6-(2-methyl-6-(trifluoromethyl) morpholino)pyridin-3-yl)spiro[3.3]heptane-2,6-diamine

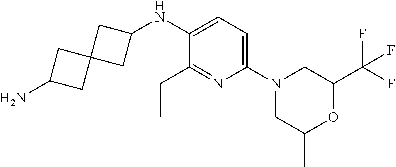 [SMILES: CCc1nc(N2CC(C)OC(C(F)(F)F)C2)ccc1NC1CC2(CC(N)C2)C1]

The titled compound 659 (4 mg, 15.5%) as a yellow solid was prepared according to the procedure outlined for compound 24. ¹H NMR (400 MHz, CD₃OD) δ 6.86 (d, J=8.6 Hz, 1H), 6.54 (d, J=8.6 Hz, 1H), 4.16-4.08 (m, 2H), 3.88-3.66 (m, 3H), 3.49-3.36 (m, 1H), 2.66-2.21 (m, 8H), 1.95-1.83 (m, 4H), 1.24-1.09 (m, 6H). MS (ESI) m/z 399.1 [M+H]⁺

### Compound 660

### N⁵-(6-aminospiro[3.3]heptan-2-yl)-N²-(2-methoxyethyl)-6-methyl-N2-(2,2,2-trifluoroethyl)pyridine-2,5-diamine

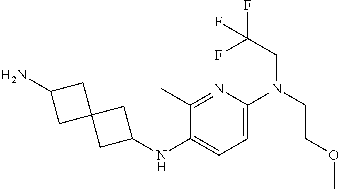 [SMILES: COCCN(CC(F)(F)F)c1ccc(NC2CC3(CC(N)C3)C2)c(C)n1]

The titled compound 660 (21.2 mg, 26.0%) as a yellow solid was prepared according to the procedure outlined for compound 24. ¹H NMR (400 MHZ, DMSO-d₆) δ 6.73 (d, J=8.8, 1H), 6.50 (d, J=8.8 Hz, 1H), 4.33 (q, J=9.6 Hz, 2H), 3.64-3.54 (m, 3H), 3.46 (t, J=6.0 Hz, 2H), 3.23 (s, 3H), 3.14 (q, J=7.6 Hz, 1H), 2.43-2.35 (m, 1H), 2.35-2.29 (m, 1H), 2.27-2.21 (m, 1H), 2.19 (s, 3H), 2.15-2.07 (m, 1H), 1.88-1.78 (m, 2H), 1.70-1.58 (m, 2H). Mass (m/z): 346.3 [M+H]⁺.

### Compound 661

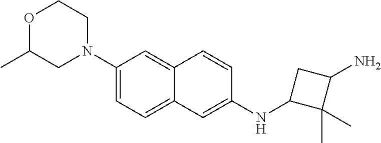 [SMILES: CC1CN(c2ccc3cc(NC4CC(N)C4(C)C)ccc3c2)CCO1]

The titled compound 661 (51.7 mg, 77.8%) as a white solid was prepared according to the procedure outlined for compound 24. ¹H NMR (400 MHZ, DMSO-d₆) δ 7.64-7.28 (m, 4H), 7.22-7.13 (m, 1H), 7.02-6.88 (m, 2H), 6.64-6.49 (m, 1H), 5.98-5.79 (m, 1H), 3.96-3.80 (m, 2H), 3.74-3.62 (m, 2H), 3.59-3.43 (m, 3H), 3.26-3.17 (m, 1H), 2.68-2.54 (m, 1H), 2.39-2.27 (m, 2H), 2.26-2.16 (m, 1H), 1.28 (d, J=7.7 Hz, 3H), 1.17 (d, J=6.2 Hz, 3H), 0.97 (d, J=10.2 Hz, 3H). Mass (m/z): 340.2 [M+H]⁺.

### Example 2. Determination of EC50 Values in HT-1080 Cell Viability Assay

HT-1080 (ATCC, CCL-121) cells (6000 in 100 μl) were seeded in 96-well plates (Corning) and cultured in a 37° C. incubator with a humidified atmosphere of 5% CO₂ for overnight. Cells were then treated with ferroptosis inducer RSL3 (TargetMol) and a 3-fold, 10-point serial dilution series of compounds with 1.1 μM as the highest concentration, for 20 hours. Cell viability was assessed using the Cell Titer-Glo Kit (Promega). The luminescence intensity was measured with a microplate reader (Envision, PerkinElmer), and cell viability was calculated by normalizing the data to untreated controls. The EC50 values of the compounds (e.g., Compounds 1-573 and 574-661) were calculated in GraphPad Prism (GraphPad Software, Inc., San Diego, CA, USA). The curves were fitted using a non-linear regression model with a sigmoidal dose response.

Ferroptosis inhibitory activity of compounds 1-573 is summarized in Table 2. In Table 2, activity is provided as follows: +++=0.1 nM≤EC50<100 nM; ++=100 nM≤EC50<1000 nM; +=1000 nM≤EC50<10000 nM.

TABLE 2
EC50 Values of Compounds 1 to 573

| Cmpd No. | EC₅₀ (nM) |
|---|---|
| 1 | +++ |
| 2 | +++ |
| 3 | +++ |
| 4A/B | +++/+++ |
| 5 | +++ |
| 6A/B | +++/+++ |
| 7 | +++ |
| 8 | +++ |
| 9 | + |
| 10 | +++ |
| 11 | ++ |
| 12 | +++ |
| 13 | +++ |
| 14 | +++ |
| 15 | +++ |
| 16 | +++ |
| 17 | +++ |
| 18 | +++ |
| 19 | +++ |
| 20 | +++ |
| 21 | +++ |
| 22 | ++ |
| 23A/B | +++/+++ |
| 24A/B | +++/+++ |
| 25 | +++ |
| 26 | +++ |
| 27 | ++ |
| 28 | +++ |
| 29 | +++ |
| 30 | +++ |
| 31 | +++ |
| 32A/B | +++/+++ |
| 33A/B | +/+ |
| 34A/B | +++/+++ |
| 35 | +++ |
| 36 | +++ |
| 37A/B | +/+ |
| 38 | +++ |
| 39 | +++ |
| 40 | +++ |
| 41A/B | ++/++ |
| 42 | +++ |
| 43 | +++ |
| 44 | +++ |
| 45 | +++ |
| 46 | ++ |
| 47 | ++ |
| 48 | +++ |
| 49 | +++ |
| 50 | +++ |
| 51 | +++ |
| 52 | +++ |
| 53A/B | +++/+++ |
| 54A/B | +++/+++ |
| 55 | +++ |
| 56 | +++ |
| 57 | +++ |
| 58 | +++ |
| 59 | +++ |
| 60 | +++ |
| 61 | +++ |
| 62 | +++ |
| 63 | +++ |
| 64A/B | +/+ |
| 65A/B | +++/+++ |
| 66 | +++ |
| 67 | +++ |
| 68 | +++ |
| 69 | +++ |
| 70 | +++ |
| 71 | +++ |
| 72 | +++ |
| 73 | +++ |
| 74 | +++ |
| 75 | ++ |
| 76A/B | +++/+++ |
| 77 | +++ |
| 78 | +++ |
| 79 | ++ |
| 80 | ++ |
| 81A/B | ++/++ |
| 82A/B | +++/+++ |
| 83 | +++ |
| 84 | +++ |
| 85 | +++ |
| 86 | +++ |
| 87 | +++ |
| 88 | + |
| 89 | +++ |
| 90 | +++ |
| 91A/B | +++/+++ |
| 92A/B | +/++ |
| 93 | +++ |
| 94 | +++ |
| 95 | +++ |
| 96 | +++ |
| 97A/B | +++/+++ |
| 98 | +++ |
| 99 | +++ |
| 100 | ++ |
| 101 | +++ |
| 102 | +++ |
| 103 | ++ |
| 104 | ++ |
| 105 | +++ |
| 106A/B | +++/+++ |
| 107A/B | +++/+++ |
| 108 | ++ |
| 109 | ++ |
| 110 | ++ |
| 111 | ++ |
| 112 | ++ |
| 113 | ++ |
| 114A/B | ++/++ |
| 115A/B | ++/++ |
| 116 | +++ |
| 117A/B | +++/+++ |
| 118 | +++ |
| 119 | +++ |
| 120A/B | +++/+++ |
| 121A/B | +++/+++ |
| 122 | +++ |
| 123 | ++ |
| 124 | ++ |
| 125 | ++ |
| 126 | + |
| 127 | ++ |
| 128 | ++ |
| 129 | ++ |
| 130 | ++ |
| 131 | +++ |
| 132 | +++ |
| 133 | +++ |
| 134 | + |
| 135A/B | ++/++ |
| 136 | +++ |
| 137 | +++ |
| 138 | ++ |
| 139A/B | +++/+++ |
| 140 | +++ |
| 141 | ++ |
| 142A/B | +++/+++ |
| 143A/B | ++/++ |
| 144A/B | ++/++ |
| 145 | +++ |
| 146A/B | +++/+++ |
| 147 | +++ |
| 148A/B | +++/+++ |
| 149A/B | ++/++ |
| 150 | ++ |
| 151 | +++ |
| 152 | ++ |
| 153A/B | ++/++ |
| 154 | ++ |
| 155 | +++ |
| 156A/B/C | +++ |
| 157A/B | +++/+++ |
| 158 | ++ |
| 159A/B | +++/++ |
| 160 | + |
| 161 | + |
| 162 | ++ |
| 163 | + |
| 164 | + |
| 165 | + |
| 166 | + |
| 167 | ++ |
| 168A/B | +/+ |
| 169 | ++ |
| 170 | + |
| 171 | +++ |
| 172 | +++ |
| 173 | ++ |
| 174 | +++ |
| 175 | + |
| 176 | ++ |
| 177 | + |
| 178 | + |
| 179 | + |
| 180 | ++ |
| 181 | ++ |
| 182A/B | ++/++ |
| 183A/B | +/+ |
| 184 | ++ |
| 185 | + |
| 186 | ++ |
| 187 | + |
| 188 | ++ |
| 189 | + |
| 190 | ++ |
| 191A/B | +/+ |
| 192 | ++ |
| 193A/B | ++/++ |
| 194 | ++ |
| 195 | ++ |
| 196 | +++ |
| 197 | +++ |
| 198 | ++ |
| 199 | + |
| 200A/B | +++/+++ |
| 201 | ++ |
| 202 | + |
| 203 | ++ |
| 204 | ++ |
| 205A/B | +++/+++ |
| 206 | + |
| 207 | + |
| 208 | + |
| 209 | +++ |
| 210 | + |
| 211 | + |
| 212 | + |
| 213A/B | ++/++ |
| 214 | +++ |
| 215 | +++ |
| 216A/B | ++/++ |
| 217 | +++ |
| 218 | ++ |
| 219 | + |
| 220 | +++ |
| 221 | +++ |
| 222 | + |
| 223 | + |
| 224 | ++ |
| 225 | +++ |
| 226 | +++ |
| 227 | + |
| 228A/B | +++/+++ |
| 229 | + |
| 230 | +++ |
| 231A/B | +++/+++ |
| 232A/B | +++/+++ |
| 233A/B | ++/++ |
| 234 | + |
| 235 | +++ |
| 236 | +++ |
| 237 | + |
| 238 | +++ |
| 239 | +++ |
| 240 | ++ |
| 241 | +++ |
| 242A/B | +++/+++ |
| 243 | +++ |
| 244A/B | +++/+++ |
| 245 | + |
| 246 | ++ |
| 247 | +++ |
| 248 | ++ |
| 249 | +++ |
| 250A/B | +++/+++ |
| 251A/B | +++/+++ |
| 252A/B/C/D | ++/+++/+++/+++ |
| 253A/B | +++/+++ |
| 254 | +++ |
| 255 | + |
| 256 | + |
| 257 | +++ |
| 258 | + |
| 259A/B | ++/++ |
| 260 | +++ |
| 261 | ++ |
| 262 | ++ |
| 263 | ++ |
| 264 | +++ |
| 265 | +++ |
| 266A/B | ++/++ |
| 267 | + |
| 268 | +++ |
| 269 | ++ |
| 270 | ++ |
| 271 | +++ |
| 272 | +++ |
| 273 | +++ |
| 274 | +++ |
| 275A/B | +++/+++ |
| 276 | +++ |
| 277 | +++ |
| 278 | +++ |
| 279 | +++ |
| 280 | +++ |
| 281 | +++ |
| 282 | ++ |
| 283 | +++ |
| 284 | +++ |
| 285 | +++ |
| 286 | +++ |
| 287 | +++ |
| 288 | +++ |
| 289A/B | +++/++ |
| 290 | +++ |
| 291 | +++ |
| 292 | +++ |
| 293 | ++ |
| 294A/B | +++/+++ |
| 295A/B | ++/+++ |
| 296A/B | +++/+++ |
| 297 | +++ |
| 298 | ++ |
| 299A/B | +++/+++ |
| 300A/B | +++/+++ |
| 301A/B | +++/+++ |
| 302 | +++ |
| 303 | +++ |
| 304 | ++ |
| 305A/B | +++/+++ |
| 306A/B | ++/++ |
| 307 | +++ |
| 308 | +++ |
| 309 | +++ |
| 310 | +++ |
| 311 | + |
| 312 | +++ |
| 313A/B | +++/+++ |
| 314 | +++ |
| 315 | +++ |
| 316 | +++ |
| 317 | +++ |
| 318A/B | +++/+++ |
| 319A/B | ++/++ |
| 320A/B | ++/++ |
| 321 | ++ |
| 322 | +++ |
| 323 | +++ |
| 324 | +++ |
| 325A/B | +++/+++ |
| 326 | +++ |
| 327 | ++ |
| 328 | +++ |
| 329 | +++ |
| 330A/B | +++/+++ |
| 331 | ++ |
| 332 | +++ |
| 333 | +++ |
| 334 | ++ |
| 335A/B | ++/+++ |
| 336A/B | +++/+++ |
| 337A/B | +++/+++ |
| 338 | +++ |
| 339 | +++ |
| 340 | +++ |
| 341 | +++ |
| 342 | +++ |
| 343 | +++ |
| 344 | +++ |
| 345 | ++ |
| 346 | +++ |
| 347 | +++ |
| 348 | +++ |
| 349A/B | +++/+++ |
| 350 | +++ |
| 351 | +++ |
| 352 | + |
| 353 | +++ |
| 354 | +++ |
| 355 | +++ |
| 356 | +++ |
| 357 | +++ |
| 358 | +++ |
| 359 | +++ |
| 360 | +++ |
| 361 | +++ |
| 362 | +++ |
| 363 | +++ |
| 364 | +++ |
| 365 | +++ |
| 366 | +++ |
| 367 | +++ |
| 368 | +++ |
| 369 | +++ |
| 370 | +++ |
| 371 | +++ |
| 372 | +++ |
| 373 | +++ |
| 374 | +++ |
| 375 | +++ |
| 376 | +++ |
| 377 | +++ |
| 378 | +++ |
| 379 | +++ |
| 380 | +++ |
| 381 | +++ |
| 382 | +++ |
| 383 | +++ |
| 384 | ++ |
| 385 | ++ |
| 386 | +++ |
| 387 | +++ |
| 388 | +++ |
| 389 | +++ |
| 390 | +++ |
| 391 | +++ |
| 392 | +++ |
| 393 | +++ |
| 394 | +++ |
| 395 | +++ |
| 396 | +++ |
| 397 | +++ |
| 398 | +++ |
| 399 | +++ |
| 400 | + |
| 401 | +++ |
| 402 | +++ |
| 403 | +++ |
| 404 | +++ |
| 405 | +++ |
| 406 | +++ |
| 407 | +++ |
| 408 | +++ |
| 409 | +++ |
| 410 | +++ |
| 411 | +++ |
| 412 | +++ |
| 413 | +++ |
| 414 | +++ |
| 415 | +++ |
| 416 | +++ |
| 417A/B | +++/++ |
| 418 | +++ |
| 419 | +++ |
| 420 | +++ |
| 421A/B | +++/+++ |
| 422 | +++ |
| 423 | +++ |
| 424 | +++ |
| 425 | +++ |
| 426 | +++ |
| 427 | +++ |
| 428 | +++ |
| 429 | +++ |
| 430 | +++ |
| 431 | +++ |
| 432 | +++ |
| 433 | +++ |
| 434 | + |
| 435 | +++ |
| 436 | +++ |
| 437 | +++ |
| 438 | +++ |
| 439 | ++ |
| 440 | ++ |
| 441 | ++ |
| 442 | ++ |
| 443 | ++ |
| 444 | +++ |
| 445 | +++ |
| 446A/B | +++/+++ |
| 447 | +++ |
| 448 | +++ |
| 449 | + |
| 450 | ++ |
| 451 | +++ |
| 452 | +++ |
| 453 | +++ |
| 454 | +++ |
| 455A/B | ++/++ |
| 456 | ++ |
| 457 | ++ |
| 458 | ++ |
| 459 | +++ |
| 460 | +++ |
| 461 | +++ |
| 462 | + |
| 463 | +++ |
| 464 | +++ |
| 465 | +++ |
| 466 | ++ |
| 467 | +++ |
| 468 | +++ |
| 469 | ++ |
| 470 | +++ |
| 471 | +++ |
| 472 | +++ |
| 473 | +++ |
| 474A/B | +++/+++ |
| 475A/B | +++/+++ |
| 476 | +++ |
| 477 | ++ |
| 478 | +++ |
| 479 | +++ |
| 480 | ++ |
| 481 | ++ |
| 482 | +++ |
| 483 | +++ |
| 484 | + |
| 485 | +++ |
| 486 | +++ |
| 487 | ++ |
| 488A/B | +++/+++ |
| 489 | +++ |
| 490 | +++ |
| 491 | +++ |
| 492 | +++ |
| 493 | +++ |
| 494 | ++ |
| 495 | ++ |
| 496 | ++ |
| 497 | +++ |
| 498 | +++ |
| 499 | ++ |
| 500 | +++ |
| 501 | + |
| 502 | +++ |
| 503 | +++ |
| 504 | +++ |
| 505 | +++ |
| 506 | +++ |
| 507 | ++ |
| 508 | +++ |
| 509 | +++ |
| 510 | +++ |
| 511 | +++ |
| 512 | +++ |
| 513 | +++ |
| 514 | +++ |
| 515 | +++ |
| 516A/B | +++/+++ |
| 517 | ++ |
| 518 | +++ |
| 519 | +++ |
| 520 | +++ |
| 521 | +++ |
| 522 | +++ |
| 523 | + |
| 524 | ++ |
| 525 | ++ |
| 526 | +++ |
| 527 | +++ |
| 528 | ++ |
| 529 | +++ |
| 530 | + |
| 531 | +++ |
| 532 | ++ |
| 533 | +++ |
| 534 | +++ |
| 535 | +++ |
| 536 | +++ |
| 537 | ++ |
| 538A/B | +++/+++ |
| 539A/B | +++/++ |
| 540 | +++ |
| 541 | +++ |
| 542 | +++ |
| 543 | +++ |
| 544 | +++ |
| 545 | +++ |
| 546 | +++ |
| 547 | +++ |
| 548 | +++ |
| 549 | +++ |
| 550 | +++ |
| 551 | ++ |
| 552 | +++ |
| 553 | ++ |
| 554 | +++ |
| 555 | +++ |
| 556 | +++ |
| 557 | +++ |
| 558 | +++ |
| 559 | +++ |
| 560 | +++ |
| 561 | +++ |
| 562 | +++ |
| 563 | +++ |
| 564 | +++ |
| 565 | +++ |
| 566 | +++ |
| 567 | +++ |
| 568 | +++ |
| 569 | +++ |
| 570 | +++ |
| 571 | +++ |
| 572 | +++ |
| 573 | +++ |

Ferroptosis inhibitory activity of compounds 574-661 is summarized in Table 3. In Table 3, activity is provided as follows: +++=0.1 nM≤EC50<100 nM; ++=100 nM≤EC50<1000 nM; +=1000 nM≤FC50<10000 nM.

TABLE 3
EC₅₀ Values of Compounds 574 to 661

| Cmpd No. | EC₅₀ (nM) |
|---|---|
| 574 | +++ |
| 575 | +++ |
| 576 | +++ |
| 577 | +++ |
| 578 | +++ |
| 579 | +++ |
| 580 | +++ |
| 581 | +++ |
| 582 | +++ |
| 583 | +++ |
| 584 | +++ |
| 585 | +++ |
| 586 | +++ |
| 587 | ++ |
| 588 | +++ |
| 589 | +++ |
| 590 | ++ |
| 591 | ++ |
| 592 | +++ |
| 593 | +++ |
| 594 | +++ |
| 595 | +++ |
| 596 | +++ |
| 597 | +++ |
| 598 | +++ |
| 599 | ++ |
| 600 | ++ |
| 601 | +++ |
| 602 | +++ |
| 603 | +++ |
| 604 | +++ |
| 605 | +++ |
| 606 | +++ |
| 607 | ++ |
| 608 | +++ |
| 609 | +++ |
| 610 | +++ |
| 611 | +++ |
| 612 | +++ |
| 613 | +++ |
| 614 | +++ |
| 615 | +++ |
| 616 | +++ |
| 617 | +++ |
| 618 | ++ |
| 619 | +++ |
| 620 | +++ |
| 621 | +++ |
| 622 | +++ |
| 623 | +++ |
| 624 | +++ |
| 625 | +++ |
| 626 | +++ |
| 627 | ++ |
| 628 | +++ |
| 629 | +++ |
| 630 | +++ |
| 631 | +++ |
| 632 | +++ |
| 633 | +++ |
| 634 | +++ |
| 635 | +++ |
| 636 | +++ |
| 637 | +++ |
| 638 | +++ |
| 639 | +++ |
| 640 | +++ |
| 641 | +++ |
| 642 | +++ |
| 643 | +++ |
| 644 | +++ |
| 645 | +++ |
| 646 | +++ |
| 647 | +++ |
| 648 | +++ |
| 649 | +++ |
| 650 | +++ |
| 651 | +++ |
| 652 | +++ |
| 653 | +++ |
| 654 | +++ |
| 655 | +++ |
| 656 | +++ |
| 657 | +++ |
| 658 | +++ |
| 659 | +++ |
| 660 | +++ |
| 661 | +++ |

The present disclosure involves the following technical solutions:

1. A compound of the following structural Formula I:

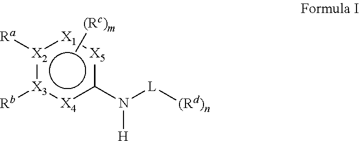

a tautomer thereof, a solvate or stereoisomer of the compound or the tautomer, or a pharmaceutically acceptable salt of the foregoing, wherein:
L is cycloalkyl, heterocyclyl, or C;
X₁, X₂, X₃, X₄, and X₅ are each independently C or N;
R^{a} is absent or selected from H, halogen, CN, optionally substituted heteroatom, optionally substituted alkyl, optionally substituted alkenyl, optionally substituted alkynyl, optionally substituted acyl, optionally substituted cycloalkyl, optionally substituted heterocyclyl, optionally substituted aryl, and optionally substituted heteroaryl;
R^{b} is absent or selected from H, halogen, CN, optionally substituted heteroatom, optionally substituted alkyl, optionally substituted cycloalkyl, and optionally substituted heterocyclyl;
R^{a} and R^{b} may join together to form an optionally substituted 3- to 10-membered carbocyclic, heterocyclic, aromatic, or heteroaromatic ring;
R^{c}, for each occurrence, is independently selected from halogen, optionally substituted heteroatom, and optionally substituted alkyl;
R^{b} and one of R^{c} may join together to form an optionally substituted 3- to 10-membered carbocyclic, heterocyclic, aromatic, or heteroaromatic ring;
R^{d}, for each occurrence, is independently selected from halogen, CN, optionally substituted heteroatom, optionally substituted alkyl, optionally substituted alkenyl, optionally substituted alkynyl, optionally substituted acyl, optionally substituted cycloalkyl, optionally substituted heterocyclyl, optionally substituted aryl, and optionally substituted heteroaryl; and m and n are each an integer independently selected from 0, 1, 2, and 3.

2. The compound of technical solution 1, wherein the compound has the following structural Formula IIa:

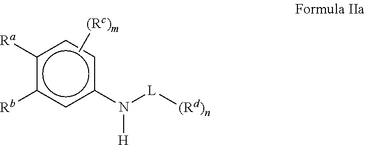

a tautomer thereof, a solvate or stereoisomer of the compound or the tautomer, or a pharmaceutically acceptable salt of the foregoing.

3. The compound of technical solution 1, wherein the compound has the following structural Formula IIb:

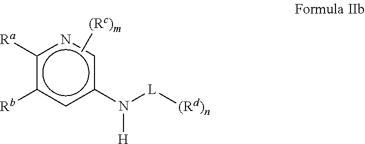

a tautomer thereof, a solvate or stereoisomer of the compound or the tautomer, or a pharmaceutically acceptable salt of the foregoing.

4. The compound of technical solution 1, wherein the compound has the following structural Formula IIc:

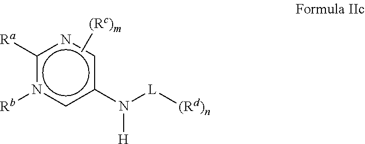

a tautomer thereof, a solvate or stereoisomer of the compound or the tautomer, or a pharmaceutically acceptable salt of the foregoing.

5. The compound of technical solution 1, wherein the compound has one of the following structural Formulae IId:

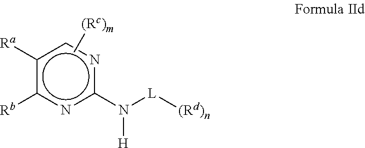

a tautomer thereof, a solvate or stereoisomer of the compound or the tautomer, or a pharmaceutically acceptable salt of the foregoing.

6. The compound of technical solution 1, wherein the compound has one of the following structural Formulae IIe:

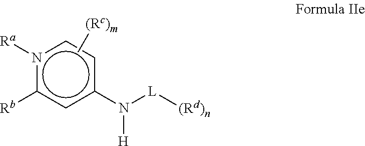

a tautomer thereof, a solvate or stereoisomer of the compound or the tautomer, or a pharmaceutically acceptable salt of the foregoing.

7. The compound of technical solution 1, wherein the compound has one of the following structural Formulae IIf:

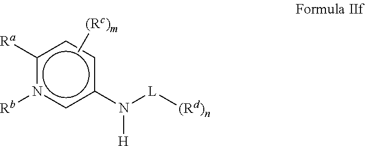

a tautomer thereof, a solvate or stereoisomer of the compound or the tautomer, or a pharmaceutically acceptable salt of the foregoing.

8. The compound of technical solution 1, wherein the compound has one of the following structural Formulae IIg:

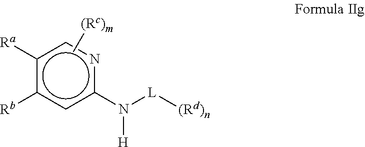

a tautomer thereof, a solvate or stereoisomer of the compound or the tautomer, or a pharmaceutically acceptable salt of the foregoing.

9. The compound of technical solution 1, wherein the compound has one of the following structural Formulae IIh:

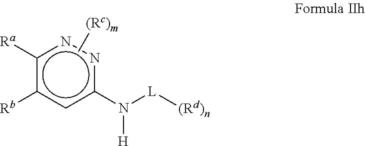

a tautomer thereof, a solvate or stereoisomer of the compound or the tautomer, or a pharmaceutically acceptable salt of the foregoing.

10. The compound of technical solution 1, wherein the compound has one of the following structural Formulae IIi:

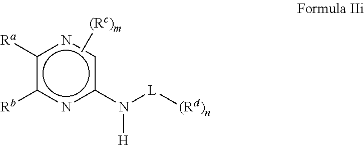

a tautomer thereof, a solvate or stereoisomer of the compound or the tautomer, or a pharmaceutically acceptable salt of the foregoing.

11. The compound of technical solution 1, wherein the compound has one of the following structural Formulae IIj:

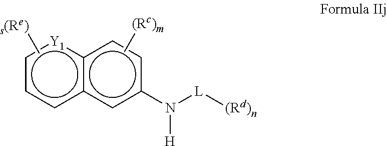

a tautomer thereof, a solvate or stereoisomer of the compound or the tautomer, or a pharmaceutically acceptable salt of the foregoing, wherein Y1 is N or C; R^{e}, for each occurrence, is independently selected from optionally substituted cycloalkyl, optionally substituted heterocyclyl, optionally substituted aryl, and optionally substituted heteroaryl; and s is an integer selected from 0, 1, 2, and 3.

12. The compound of technical solution 1, wherein the compound has the following structural Formula IIIa:

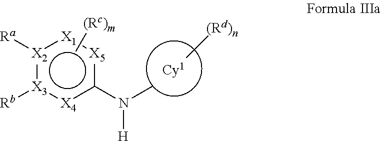

a tautomer thereof, a solvate or stereoisomer of the compound or the tautomer, or a pharmaceutically acceptable salt of the foregoing, wherein Cy¹ is 3- to 10-membered cycloalkyl or 3- to 10-membered heterocyclyl.

13. The compound of technical solution 1, wherein the compound has the following structural Formula IIIb:

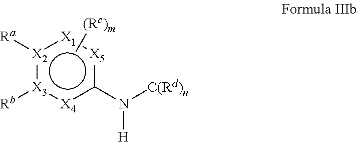

a tautomer thereof, a solvate or stereoisomer of the compound or the tautomer, or a pharmaceutically acceptable salt of the foregoing.

14. The compound of any one of technical solutions 1 and 12, wherein the compound has the following structural Formula IVa:

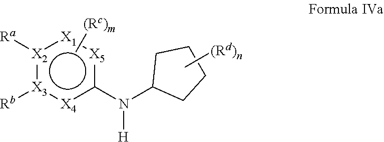

a tautomer thereof, a solvate or stereoisomer of the compound or the tautomer, or a pharmaceutically acceptable salt of the foregoing.

15. The compound of any one of technical solutions 1 and 12, wherein the compound has one of the following structural Formula IVb:

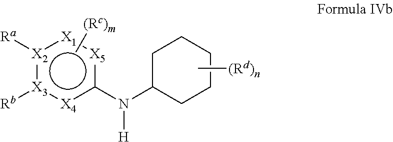

a tautomer thereof, a solvate or stereoisomer of the compound or the tautomer, or a pharmaceutically acceptable salt of the foregoing.

16. The compound of any one of technical solutions 1 and 12, wherein the compound has one of the following structural Formula IVc:

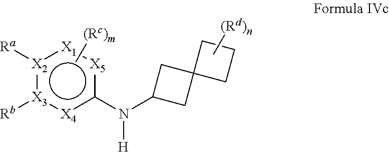

a tautomer thereof, a solvate or stereoisomer of the compound or the tautomer, or a pharmaceutically acceptable salt of the foregoing.

17. The compound of any one of technical solutions 1 and 12, wherein the compound has one of the following structural Formula IVd:

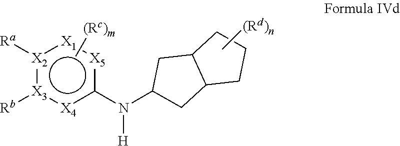

a tautomer thereof, a solvate or stereoisomer of the compound or the tautomer, or a pharmaceutically acceptable salt of the foregoing.

18. The compound of any one of technical solutions 1 and 12, wherein the compound has one of the following structural Formula IVe:

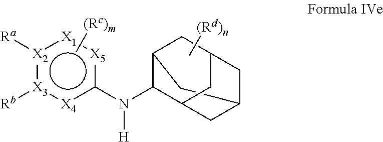

a tautomer thereof, a solvate or stereoisomer of the compound or the tautomer, or a pharmaceutically acceptable salt of the foregoing.

19. The compound of any one of technical solutions 1 and 12, wherein the compound has one of the following structural Formula IVf:

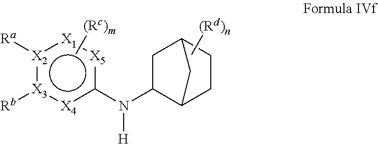

a tautomer thereof, a solvate or stereoisomer of the compound or the tautomer, or a pharmaceutically acceptable salt of the foregoing.

20. The compound of any one of technical solutions 1 and 13, wherein the compound has one of the following structural Formula Va:

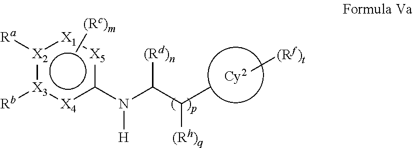

a tautomer thereof, a solvate or stereoisomer of the compound or the tautomer, or a pharmaceutically acceptable salt of the foregoing, wherein Cy² is 3- to 10-membered cycloalkyl or 3- to 10-membered heterocyclyl; R^{f}, for each occurrence, is independently selected from halogen, CN, optionally substituted heteroatom, optionally substituted alkyl, optionally substituted alkenyl, optionally substituted alkynyl, optionally substituted acyl, optionally substituted cycloalkyl, optionally substituted heterocyclyl, optionally substituted aryl, and optionally substituted heteroaryl; R^{h}, for each occurrence, is independently selected from halogen, CN, and optionally substituted C₁ to C₆ alkyl; n is an integer selected from 0, 1, and 2; p is an integer selected from 0, 1, 2, 3, 4, and 5; q is an integer selected from 0, 1, and 2; and t is an integer independently selected from 0, 1, 2, and 3.

21. The compound, tautomer, solvate, stereoisomer, or pharmaceutically acceptable salt of technical solution 20, wherein n is 0, q is 0, and p is 0, 1, 2, and 3.

22. The compound of any one of technical solutions 1, 13, and 20, wherein the compound has one of the following structural Formula VIa:

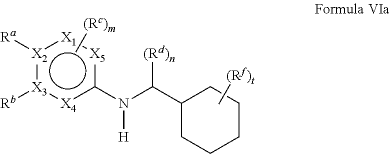

a tautomer thereof, a solvate or stereoisomer of the compound or the tautomer, or a pharmaceutically acceptable salt of the foregoing.

23. The compound of technical solution 1, wherein the compound has one of the following structural Formulae VIIa-VIIe:

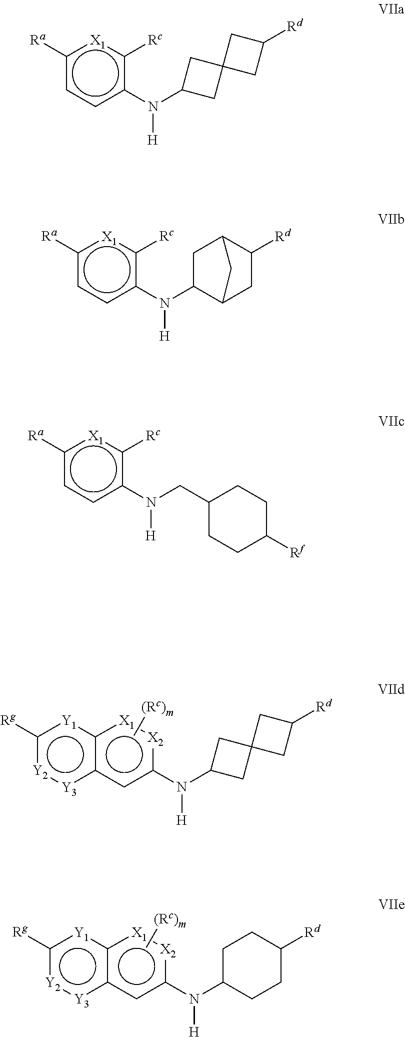

a tautomer thereof, a solvate or stereoisomer of the compound or the tautomer, or a pharmaceutically acceptable salt of the foregoing, wherein:
X₁ and X₂ are independently C or N;
Y₁, Y₂, and Y₃ are independently C or N;
R^{a} is selected from

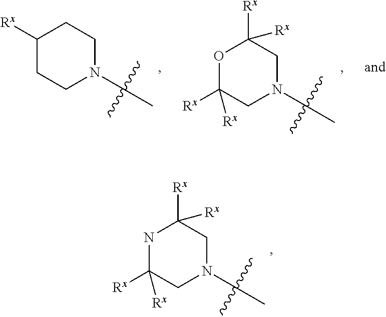

wherein R^{x}, for each occurrence, is independently selected from H, CF₃, —CH₃, and —CH₂CH₃;
R^{c} is selected from halogen, C₁ to C₂ alkyl, and C₁ to C₂ alkoxy;
R^{d} is selected from NH₂ and —C(═O)NH₂;
R^{f} is selected from NH₂ and —C(═O)NH₂; and
m is 0 or 1.

24. The compound, tautomer, solvate, stereoisomer, or pharmaceutically acceptable salt of technical solution 11, wherein R^{e}, for each occurrent, is independently selected from 5- to 6-membered cycloalkyl and 5- to 6-membered heterocyclyl, each independently substituted with 0, 1, 2, or 3 groups selected from C₁-C₆ alkyl optionally substituted with 1 to 4 groups selected from ═O, and halogen.

25. The compound, tautomer, solvate, stereoisomer, or pharmaceutically acceptable salt of any one of technical solutions 11 and 24, wherein R^{e}, for each occurrence, is independently selected from

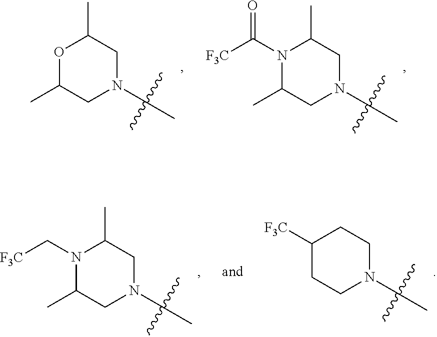

26. The compound, tautomer, solvate, stereoisomer, or pharmaceutically acceptable salt of any one of technical solutions 20-22, wherein R^{f}, for each occurrence, is independently selected from H, halogen, cyano, C₁-C₁₀ alkyl, phenyl, 5- to 7-membered heteroaryl, phenyl, 3- to 10-membered heterocyclyl, C₃-C₁₀ cycloalkyl, —C(—O) R^{s}, C₂-C₁₀ alkenyl, ═O, ═NR^{p}; —C(═O)OR^{s}, —C(═O)NR^{p}R^{q}, —NR^{p}R^{q}, and —NR^{p}C(═O)R^{s}, wherein
the C₁-C₁₀ alkyl and C₂-C₁₀ alkenyl of R^{f} are each optionally substituted with 1 to 3 groups selected from 5- to 7-membered heteroaryl, C₃-C₁₀ cycloalkyl, 3- to 10-membered heterocyclyl, —OR^{s}, —C(═O)NR^{p}R^{q}, —NR^{p}C(═O)NR^{q}R^{r}, and —NR^{p}R^{q};
the 3- to 10-membered heterocyclyl and C₃-C₁₀ cycloalkyl of R^{f} and the 3- to 10-membered heterocyclyl and C₃-C₆ cycloalkyl of the C₁-C₁₀ alkyl and C₂-C₁₀ alkenyl of R^{f}, are each optionally substituted with 1 to 3 groups selected from ═O and C₁-C₆ alkyl optionally substituted with 1 to 3 groups selected from halogen and —OR^{s};
the phenyl and 5- to 7-membered heteroaryl of R^{f} and the 5- to 7-membered heteroaryl of the C₁-C₁₀ alkyl and C₂-C₁₀ alkenyl of R^{f} are each optionally substituted with 1 to 3 groups selected from C₁-C₆ alkyl optionally substituted with 1 to 3 groups selected from halogen and —OR^{s};
R^{p}, R^{q}, and R^{r}, for each occurrence, are independently selected from hydrogen, C₃-C₁₀ cycloalkyl, and C₁-C₁₀ alkyl, wherein the C₃-C₁₀ cycloalkyl and C₁-C₁₀ alkyl of R^{p}, R^{q}, and R^{r} are each optionally substituted with 1 to 3 groups selected from halogen, —OR^{s} and —C(═O)OR^{s};
R^{s}, for each occurrence, is independently selected from H, NH₂, C₃-C₁₀ cycloalkyl, 3- to 10-membered heterocyclyl, and C₁-C₁₀ alkyl optionally substituted with 1 to 3 groups selected from halogen, C₃-C₁₀ cycloalkyl, 3- to 10-membered heterocyclyl, and C₁-C₆ alkoxy, wherein
the C₃-C₁₀ cycloalkyl and 3- to 10-membered heterocyclyl of R^{s} and the C₃-C₁₀ cycloalkyl and 3- to 10-membered heterocyclyl of the C₁-C₁₀ alkyl of R^{s} are each optionally substituted with 1 to 3 groups of C₁-C₆ alkyl optionally substituted by ═O, and halogen, and the C₁-C₆ alkoxy of the C₁-C₁₀ alkyl of R^{s} is optionally substituted with 1 to 3 groups of halogen.

27. The compound, tautomer, solvate, stereoisomer, or pharmaceutically acceptable salt of any one of 20-22 and 26, wherein R^{f}, for each occurrence, is selected from H, methyl, NH₂, —NH (CH₂)₂OH, —CH₂NH₂,

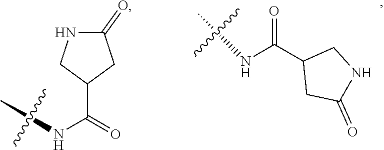

—NHC(═O)NH₂, —NHCH₃, —C(═O)NH₂, and —NHBoc.

28. The compound, tautomer, solvate, stereoisomer, or pharmaceutically acceptable salt of any one of technical solutions 1-10, 12-22, and 26-27, wherein R^{a} is absent or selected from hydrogen, halogen, cyano, C₁-C₁₀ alkyl, C₂-C₁₀ alkenyl, C₂-C₁₀ alkynyl, C₃-C₁₀ cycloalkyl, 3- to 10-membered heterocyclyl, phenyl, 5- to 7-membered heteroaryl, —C(═O)R^{s}, —C(—O)OR^{s}, —NR^{p}R^{q}, —OR^{s}, wherein the C₃-C₁₀ cycloalkyl, 3- to 10-membered heterocyclyl, phenyl, and 5- to 7-membered heteroaryl of R^{a} are each optionally substituted with 1-3 groups selected from halogen, —O, OR^{s}, and C₁-C₁₀ alkyl optionally substituted with 1 to 3 groups selected from ═O, OH, —OR^{s}, and halogen;
the C₁-C₁₀ alkyl, C₂-C₁₀ alkenyl, and C₂-C₁₀ alkynyl of R^{a} are each optionally substituted with 1 to 3 groups selected from halogen, 3- to 10-membered heterocyclyl, and —OR^{s};
R^{p} and R^{q}, for each occurrence, are independently selected from hydrogen, C₃-C₁₀ cycloalkyl, and C₁-C₁₀ alkyl, wherein the C₃-C₁₀ cycloalkyl and C₁-C₁₀ alkyl of R^{p} and R^{q} are each optionally substituted with 1 to 3 groups selected from halogen, —OR^{s} and —C(═O) OR^{s};
R^{s}, for each occurrence, is selected from H, NH₂, C₃-C₁₀ cycloalkyl, 3- to 10-membered heterocyclyl, 5- to 6-membered aryl, and C₁-C₁₀ alkyl optionally substituted with 1 to 3 groups selected from halogen, C₃-C₁₀ cycloalkyl, 3- to 10-membered heterocyclyl, and C₁-C₆ alkoxy, wherein the C₃-C₁₀ cycloalkyl and 3- to 10-membered heterocyclyl of R^{s} and the C₃-C₁₀ cycloalkyl and 3- to 10-membered heterocyclyl of the C₁-C₁₀ alkyl of R^{s} are each optionally substituted with 1 to 3 groups of C₁-C₆ alkyl optionally substituted with ═O, and halogen, and the C₁-C₆ alkoxy of the C₁-C₁₀ alkyl of R^{s} is optionally substituted with 1 to 3 groups of halogen.

29. The compound, tautomer, solvate, stereoisomer, or pharmaceutically acceptable salt of any one of technical solutions 1-10, 12-22, and 26-28, wherein R^{a} is absent or selected from H, F, Cl, Br, methyl,

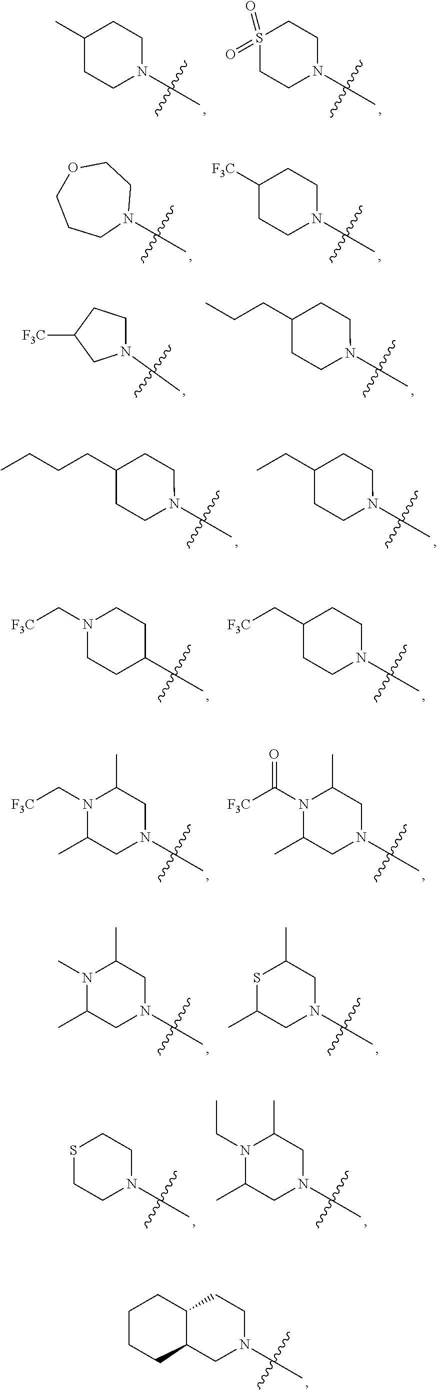

—CH(CH₂CH₃)₂, —CH(CH₃)₂, —OCH(CH₃)₂, —CH₂CH₃, —CH₂CH₂CH₃, —NHCH₂CF₃, —N(CH₃)CH₂CF₃, —O(CH₂)₃CH₃, —O(CH₂)₂CH₃, —(CH₂)₂CH₃, —(CH₂)₄CH₃, —(CH₂)₃CH₃,

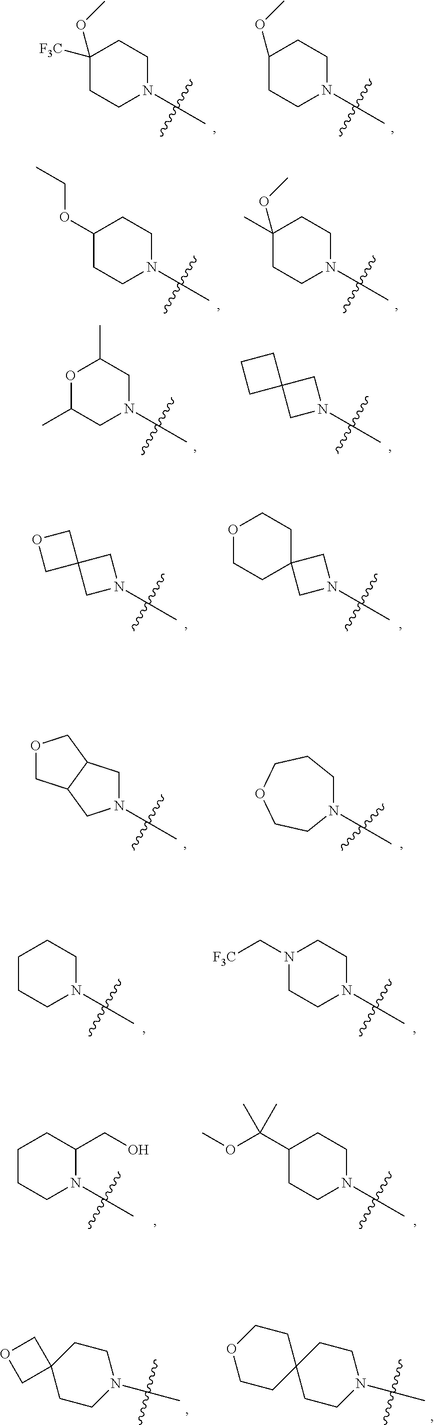

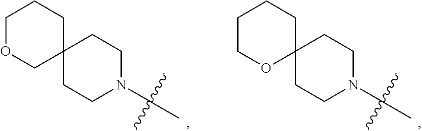 [SMILES: *N1CCC2(CCCOC2)CC1 ; *N1CCC2(CCCCO2)CC1]

—N(CH₂CH₃)(CH₂)₄CH₃, —N(CH₂CH₃)(CH₂)₃OCH₃,

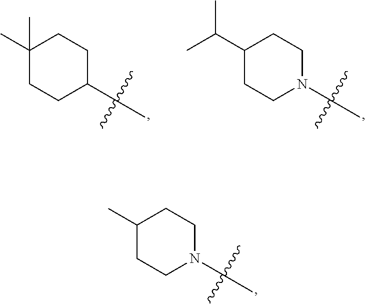

—C(CH₃)₂CH₂CH₃, —C(CH₃)₂CH₂CH₂CH₃, —C(CH₃)₂CH₂OCH₂CH₃, —C(CH₃)₃,

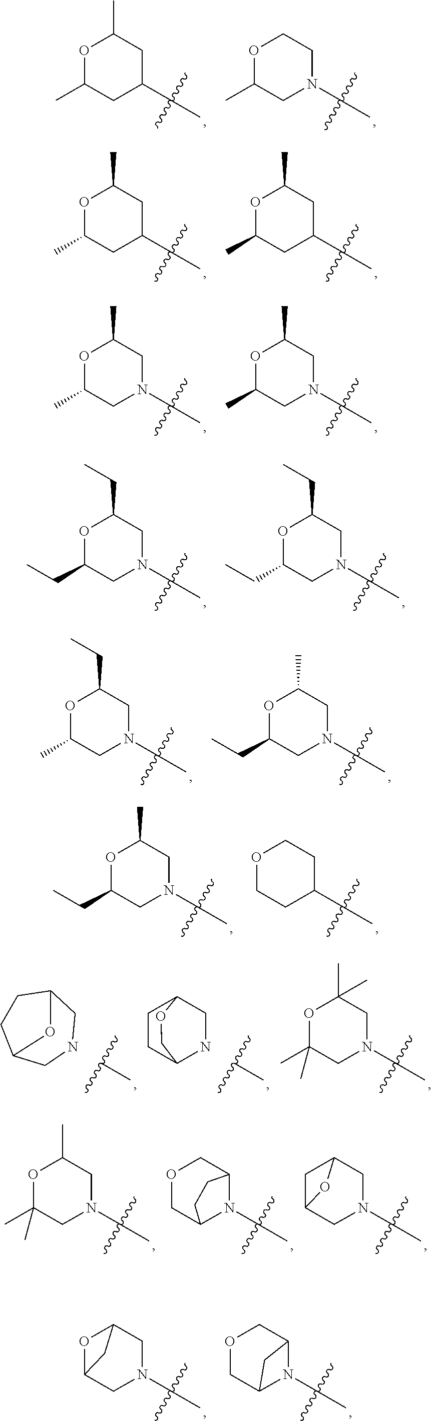

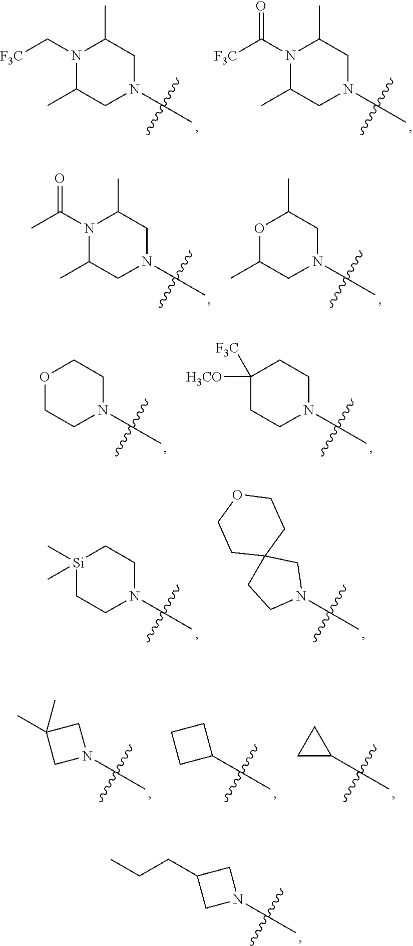

—N(CH₃)₂, —O(CH₂)₂OCH₂CH₃, —OCH₂CH₃, —(CH₂)₂OCH₃, —(CH₂)₃OCH₂CH₃,

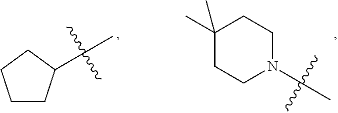

—CF₃, —CH₂CF₃, —N(CH₂CH₃)₂,

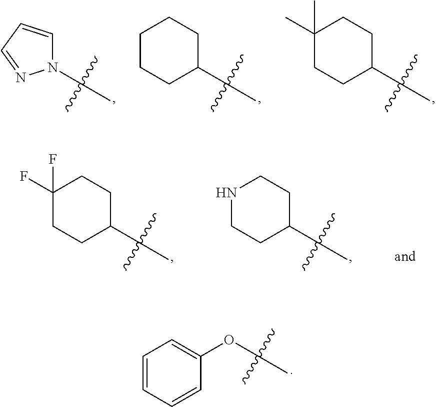

30. The compound, tautomer, solvate, stereoisomer, or pharmaceutically acceptable salt of any one of technical solutions 1-10, 12-22, and 26-28, wherein R^{a} is selected from

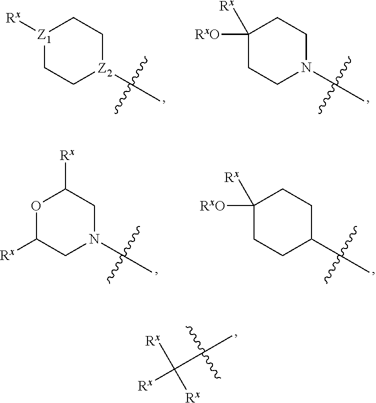

—OR^{x}, and —CH₂R^{x}, wherein Z₁ and Z₂ are independently selected from C and N, R^{x}, for each occurrence, is independently selected from C₁ to C₄ alkyl optionally substituted with 1 to 3 groups of halogen.

31. The compound, tautomer, solvate, stereoisomer, or pharmaceutically acceptable salt of any one of technical solutions 1-10, 12-22, and 26-30, wherein R^{b} is absent or selected from is H, halogen, C₁ to C₆ alkyl, —NR^{p}R^{q}, CN, C₁-C₆ alkoxy, and 5- to 6-membered heterocyclyl optionally substituted with C₁-C₃ alkyl optionally substituted with 1 to 3 groups selected from halogen, wherein R^{p} and R^{q} are each selected from C₁ to C₆ alkyl, and wherein the C₁-C₆ alkyl and C₁-C₆ alkoxy of R^{b} and the C₁-C₆ alkyl of R^{p} and R^{q} are each optionally substituted with 1 to 3 groups selected from halogen, —OR^{s}, and —NH₂.

32. The compound, tautomer, solvate, stereoisomer, or pharmaceutically acceptable salt of any one of technical solutions 1-10, 12-22, and 26-31, wherein R^{b} absent or is H, methyl, ethyl, —O(CH₂)₄OCH₃, —O(CH₂)₃OCH₃, —O(CH₂)₂OCH₃, —O(CH₂) 2NH₂, —(CH₂)₅CH₃, —O(CH₂)₅CH₃, —(CH₂)₂OCH₃, —O(CH₂)₂OH, F, —OCH₃, —CF₃, Cl, CN, —C(CH₃)₃, and

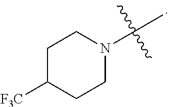 [SMILES: *N1CCC(C(F)(F)F)CC1]

33. The compound, tautomer, solvate, stereoisomer, or pharmaceutically acceptable salt of any one of technical solutions 1-10, 12-22, and 26-31, wherein R^{b} is absent or selected from halogen, C₁-C₂ alkyl, and C₁-C₂ alkoxy.

34. The compound, tautomer, solvate, stereoisomer, or pharmaceutically acceptable salt of any one of technical solutions 1-22 and 24-33, wherein R^{c}, for each occurrence, is independently selected from halogen, C₁-C₆ alkyl, —NR^{p}R^{q}, CN, and C₁-C₆ alkoxy, wherein R^{p} and R^{q} are each selected from C₁ to C₆ alkyl, and wherein the C₁-C₆ alkyl and C₁-C₆ alkoxy of R^{b} and the C₁-C₆ alkyl of R^{p} and R^{q} are each optionally substituted with 1 to 3 groups selected from halogen, —OR^{s}, and —NH₂.

35. The compound, tautomer, solvate, stereoisomer, or pharmaceutically acceptable salt of any one of technical solutions 1-22 and 24-34, wherein R^{c} is selected from methyl, ethyl, —O(CH₂)₄OCH₃, —O(CH₂)₃OCH₃, —O(CH₂)₂OCH₃, —O(CH₂)₂NH₂, —(CH₂)₅CH₃, —O(CH₂)₅CH₃, —(CH₂)₂OCH₃, —O(CH₂)₂OH, F, —OCH₃, —CF₃, Cl, CN,

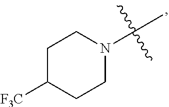 [SMILES: *N1CCC(C(F)(F)F)CC1]

and —C(CH₃)₃.

36. The compound, tautomer, solvate, stereoisomer, or pharmaceutically acceptable salt of any one of technical solutions 1-22 and 24-34, wherein R^{c} is selected from halogen, C₁-C₂ alkyl, and C₁-C₂ alkoxy.

37. The compound, tautomer, solvate, stereoisomer, or pharmaceutically acceptable salt of any one of technical solutions 1-22 and 24-36, wherein R^{d}, for each occurrence, is absent or independently selected from halogen, —OH, cyano, C₁-C₁₀ alkyl, phenyl, 5- to 7-membered heteroaryl, phenyl, 3- to 10-membered heterocyclyl, C₃-C₁₀ cycloalkyl, —C(═O) R^{s}, —SO₂R^{s}, C₂-C₁₀ alkenyl, ═O, ═NR^{p}; —C(═O)OR^{s}, —C(═O)NR^{p}R^{q}, —NR^{p}R^{q}, —NR^{p}C(═O)R^{s}, and —NR^{p}SO₂R^{s}, wherein
the C₁-C₁₀ alkyl and C₂-C₁₀ alkenyl of R^{a} are each optionally substituted with 1 to 3 groups selected from 5- to 7-membered heteroaryl, C₃-C₁₀ cycloalkyl, 3- to 10-membered heterocyclyl, —OR^{s}, —C(—O) NR^{p}R^{q}, NR^{p}C(═O)R^{s}, —NR^{p}C(═NH)R^{s}, —NR^{p}C(═O)NR^{q}R^{r}, and —NR^{p}R^{q};
the 3- to 10-membered heterocyclyl and C₃-C₁₀ cycloalkyl of R^{d} and the 3- to 10-membered heterocyclyl and C₃-C₆ cycloalkyl of the C₁-C₁₀ alkyl and C₂-C₁₀ alkenyl of R^{d}, are each optionally substituted with 1 to 3 groups selected from ═O, —NR^{p}R^{q}, NR^{p}C(═O) R^{s}, and C₁-C₆ alkyl optionally substituted with 1 to 3 groups selected from halogen and —OR^{s};
the phenyl and 5- to 7-membered heteroaryl of R^{d} and the 5- to 7-membered heteroaryl of the C₁-C₁₀ alkyl and C₂-C₁₀ alkenyl of R^{d} are each optionally substituted with 1 to 3 groups selected from C₁-C₆ alkyl optionally substituted with 1 to 3 groups selected from halogen and —OR^{s},
R^{p}, R^{q}, and R^{r}, for each occurrence, are independently selected from hydrogen, —OH, C₃-C₁₀ cycloalkyl, 5- to 7-membered heteroaryl, 5- to 6-membered aryl, and C₁-C₁₀ alkyl, wherein the C₃-C₁₀ cycloalkyl, 5- to 7-membered heteroaryl, 5- to 6-membered aryl, and C₁-C₁₀ alkyl of R^{p}, R^{q}, and R^{r} are each optionally substituted with 1 to 3 groups selected from halogen, C₁-C₆ alkyl, —OR^{s} and —C(═O)OR^{s};
R^{s}, for each occurrence, is independently selected from H, —NH₂, C₃-C₁₀ cycloalkyl, 3- to 10-membered heterocyclyl, and C₁-C₁₀ alkyl optionally substituted with 1 to 3 groups selected from halogen, ═O, NH₂, C₃-C₁₀ cycloalkyl, 3- to 10-membered heterocyclyl, and C₁-C₆ alkoxy, wherein
the C₃-C₁₀ cycloalkyl and 3- to 10-membered heterocyclyl of R^{s} and the C₃-C₁₀ cycloalkyl and 3- to 10-membered heterocyclyl of the C₁-C₁₀ alkyl of R^{s} are each optionally substituted with 1 to 3 groups selected from ═O and C₁-C₆ alkyl optionally substituted by halogen, and the C₁-C₆ alkoxy of the C₁-C₁₀ alkyl of R^{s} is optionally substituted with 1 to 3 groups of halogen.

38. The compound, tautomer, solvate, stereoisomer, or pharmaceutically acceptable salt of any one of 1-22 and 24-37, wherein R^{d}, for each occurrence, is absent or selected from

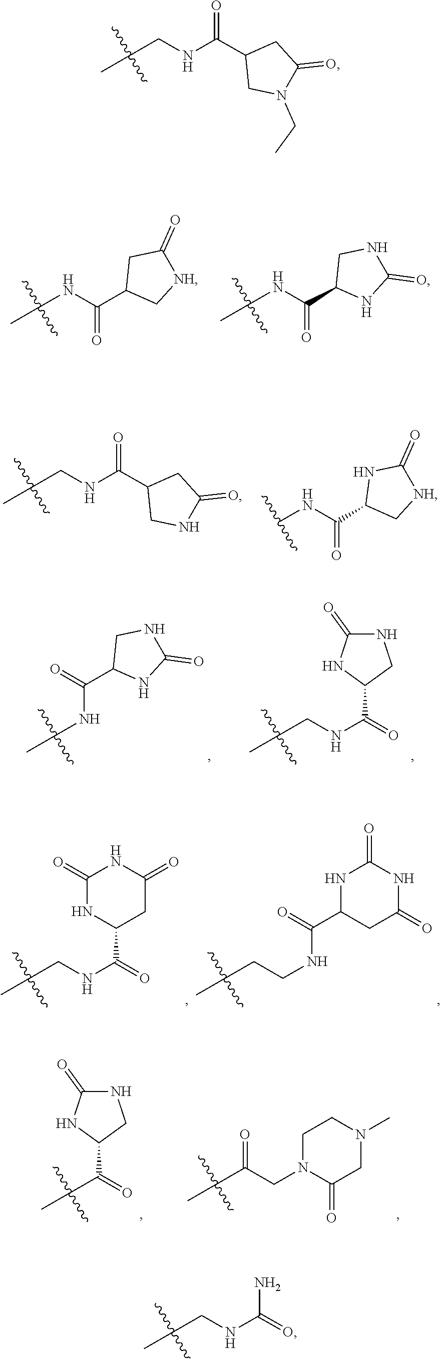

F, methyl, ethyl, propyl, butyl, —(CH₂)₃NHCH₃, —CH₂N(CH₃)₂, —CH₂N⁺(CH₃)₃, —(CH₂)₃N(CH₃)₂, —(CH₂)₃NH₂, —(CH₂)₅NH₂, —(CH₂)₄NH₂, —CH₂CH₂NH₂, —CH₂NH₂, —CH₂NHCH₃, —CHCH₃NH₂, —C(CH₃)₂NH₂, —CH₂NH(CH₂)₂OH, —(CH₂)₂NHC(═O)NH₂, —CH₂OCH₂CH₂NH₂, —CH₂CH₂OH, —CH₂OH, —CH₂OCH₃, —CH₂CH₂CH₂OCH₃, —CH₂OCH₂CH₂OCH₃, —CH₂OCH₂CH₂OH, —C(═O)N(CH₃)₂, —C(═O)NHCH₃, —C(═O)NHNH₂, —C(—O)NHOH, —CH₂OH, OH, —O(CH₂)₂N(CH₃)₂, ═O, —C(═O)OCH₃, —C(═O)OH, —C(═O)NH₂, —NH₂, —NHC(═O)CH₃, —NHC(═O)NH₂, —NH(CH₂)₂OH, —NH(CH₂)₃OH, —NH(CH₂)₄OH, —NH(CH₂)₃NH₂, —NH(CH₂)₂NH₂, —NHCH₂CH₂N(CH₃)₂, —NHCH₂CF₃, —NHCH₂CH₂F, —NHCH₂CHF₂, —NHCH₂CH₃, —NHCH₂CH₂OCH₃, —N(CH₃)₂, —NHCH₃, —NHOH, —NHSO₂NH₂, —SO₂NH₂,

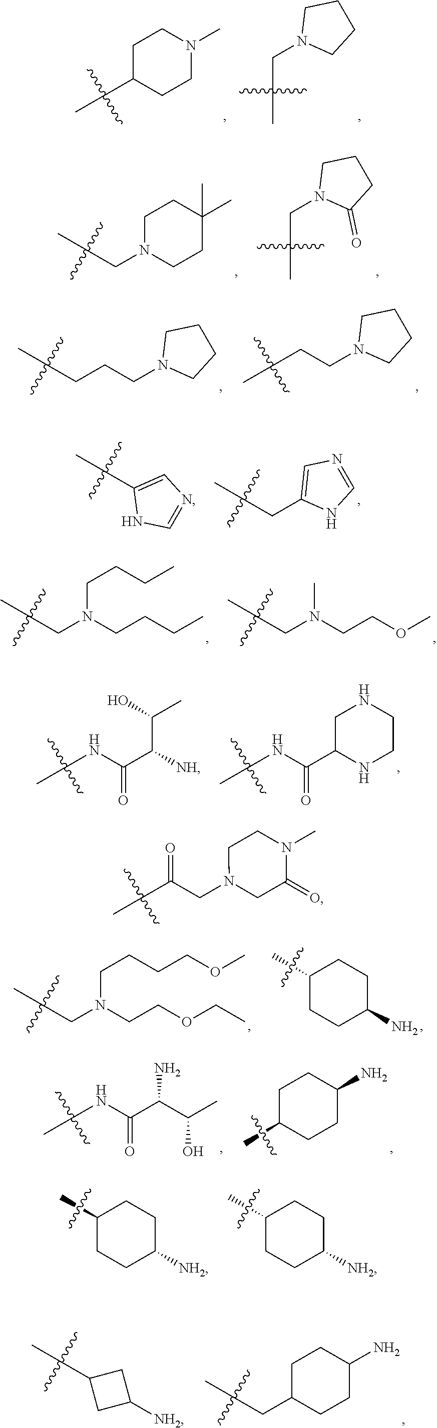

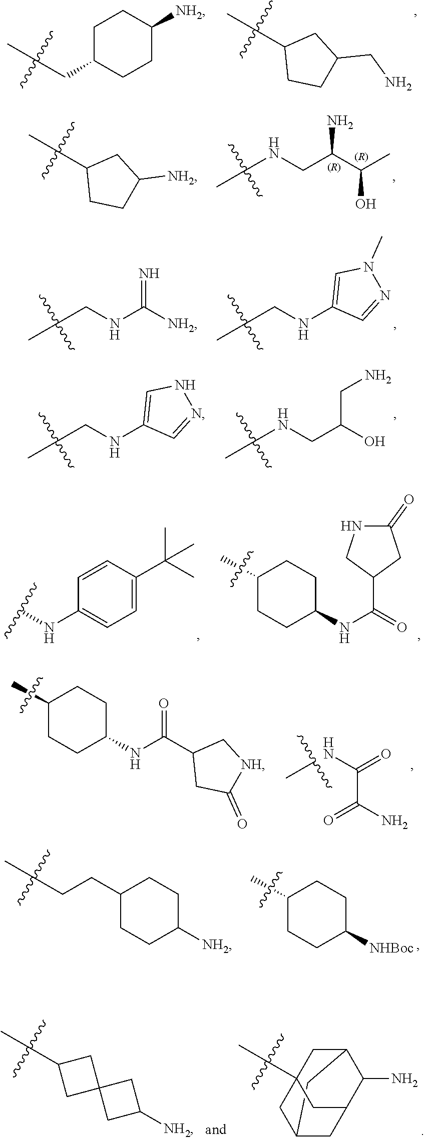

39. The compound, tautomer, solvate, stereoisomer, or pharmaceutically acceptable salt of any one of 1-22 and 24-37, wherein R^{d} is selected from —C(═O)NR^{p}R^{q}, and C₁-C₆ alkyl, wherein the C₁-C₆ alkyl is optionally substituted with 1 to 3 groups selected from C₁-C₃ alkoxy, —C(═O)NR^{p}R^{q}, and —NR^{p}R^{q}, wherein R^{p} and R^{q} are each selected from H and C₁-C₃ alkyl.

40. The compound, tautomer, solvate, stereoisomer, or pharmaceutically acceptable salt of any one of technical solutions 1-10 and 12-22, wherein R^{a} and R^{b} join to form a 5- to 6-membered carbocyclic, heterocyclic, aromatic, or heteroaromatic ring optionally substituted with 1 to 4 groups selected from optionally substituted C₁-C₆ alkyl.

41. The compound, tautomer, solvate, stereoisomer, or pharmaceutically acceptable salt of any one of technical solutions 1-10, 12-22, and 40, wherein R^{a} and R^{b} join to form a structure selected from:

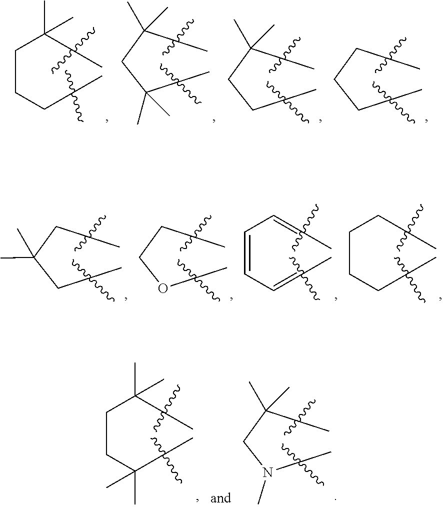

42. The compound, tautomer, solvate, stereoisomer, or pharmaceutically acceptable salt of any one of 1-10 and 12-22, wherein R^{b} and R^{c} join to form a 5- to 6-membered carbocyclic, heterocyclic, aromatic, or heteroaromatic ring optionally substituted with 1 to 4 groups selected from optionally substituted C₁-C₆ alkyl.

43. The compound, tautomer, solvate, stereoisomer, or pharmaceutically acceptable salt of any one of 1-10, 12-22, and 42, wherein R^{b} and R^{c} join to form a structure selected from:

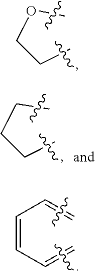

44. A compound selected from:

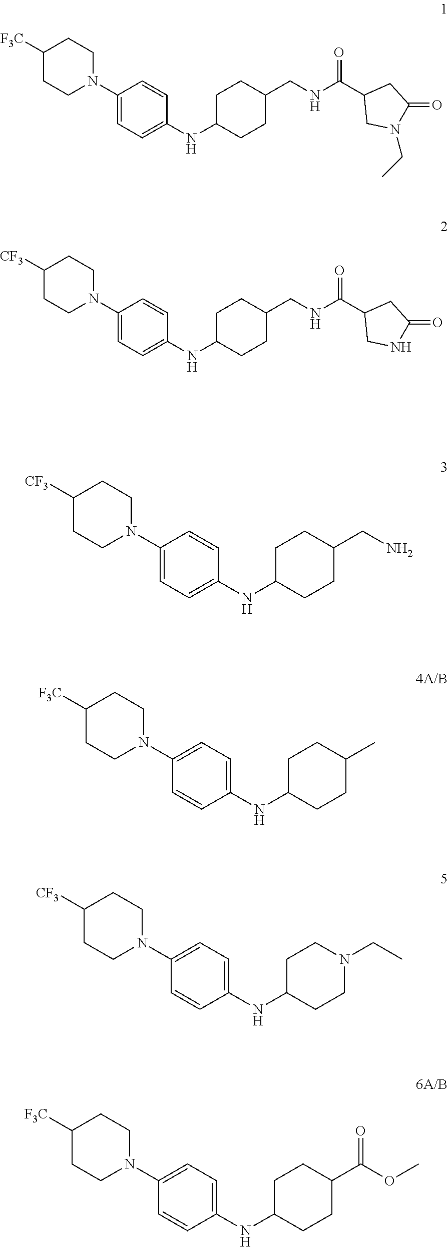 [SMILES: CCN1CC(C(=O)NCC2CCC(Nc3ccc(N4CCC(C(F)(F)F)CC4)cc3)CC2)CC1=O ; O=C1CC(C(=O)NCC2CCC(Nc3ccc(N4CCC(C(F)(F)F)CC4)cc3)CC2)CN1 ; NCC1CCC(Nc2ccc(N3CCC(C(F)(F)F)CC3)cc2)CC1 ; CC1CCC(Nc2ccc(N3CCC(C(F)(F)F)CC3)cc2)CC1 ; CCN1CCC(Nc2ccc(N3CCC(C(F)(F)F)CC3)cc2)CC1 ; COC(=O)C1CCC(Nc2ccc(N3CCC(C(F)(F)F)CC3)cc2)CC1]

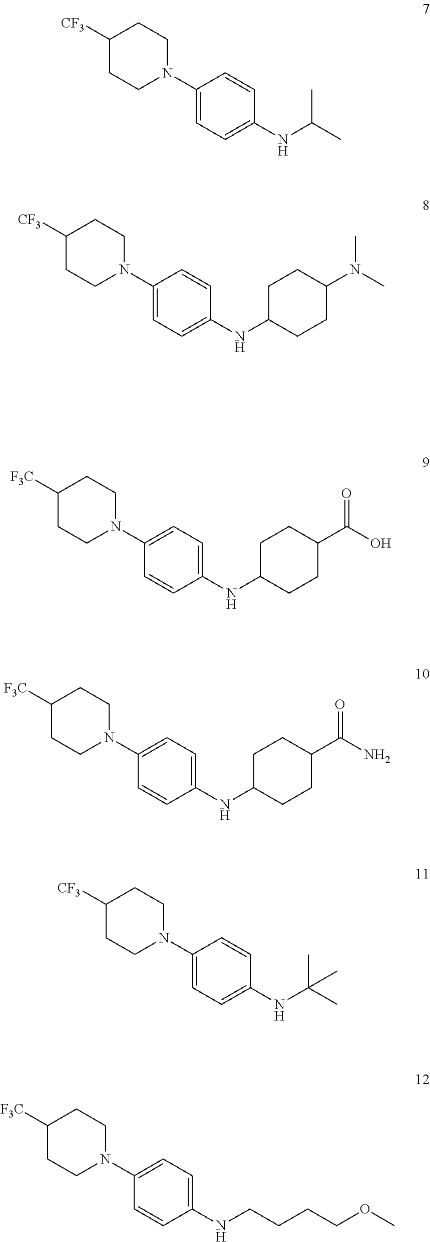 [SMILES: CC(C)Nc1ccc(N2CCC(C(F)(F)F)CC2)cc1 ; CN(C)C1CCC(Nc2ccc(N3CCC(C(F)(F)F)CC3)cc2)CC1 ; O=C(O)C1CCC(Nc2ccc(N3CCC(C(F)(F)F)CC3)cc2)CC1 ; NC(=O)C1CCC(Nc2ccc(N3CCC(C(F)(F)F)CC3)cc2)CC1 ; CC(C)(C)Nc1ccc(N2CCC(C(F)(F)F)CC2)cc1 ; COCCCCNc1ccc(N2CCC(C(F)(F)F)CC2)cc1]

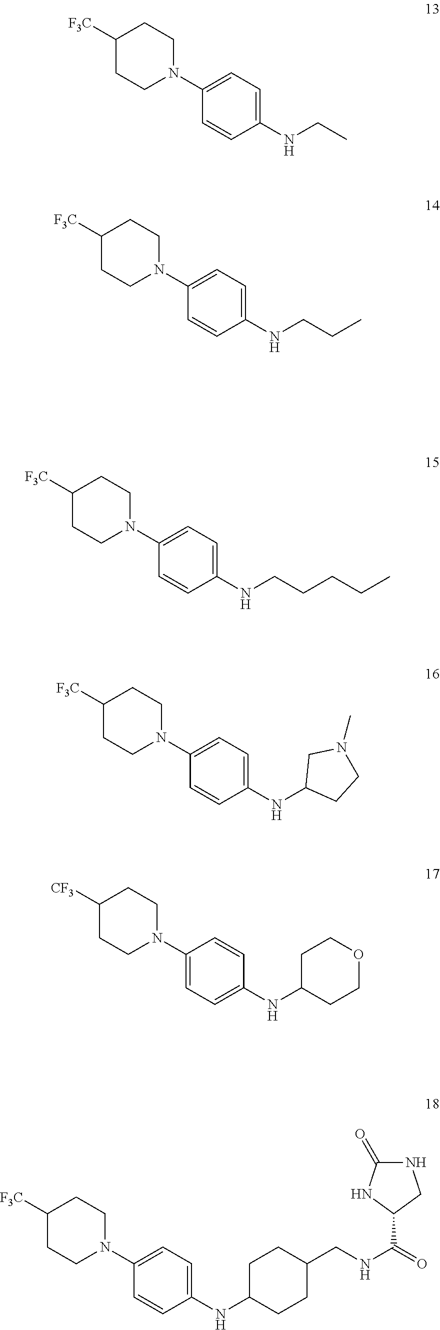 [SMILES: CCNc1ccc(N2CCC(C(F)(F)F)CC2)cc1 ; CCCNc1ccc(N2CCC(C(F)(F)F)CC2)cc1 ; CCCCCNc1ccc(N2CCC(C(F)(F)F)CC2)cc1 ; CN1CCC(Nc2ccc(N3CCC(C(F)(F)F)CC3)cc2)C1 ; FC(F)(F)C1CCN(c2ccc(NC3CCOCC3)cc2)CC1 ; O=C1NC[C@H](C(=O)NCC2CCC(Nc3ccc(N4CCC(C(F)(F)F)CC4)cc3)CC2)N1]

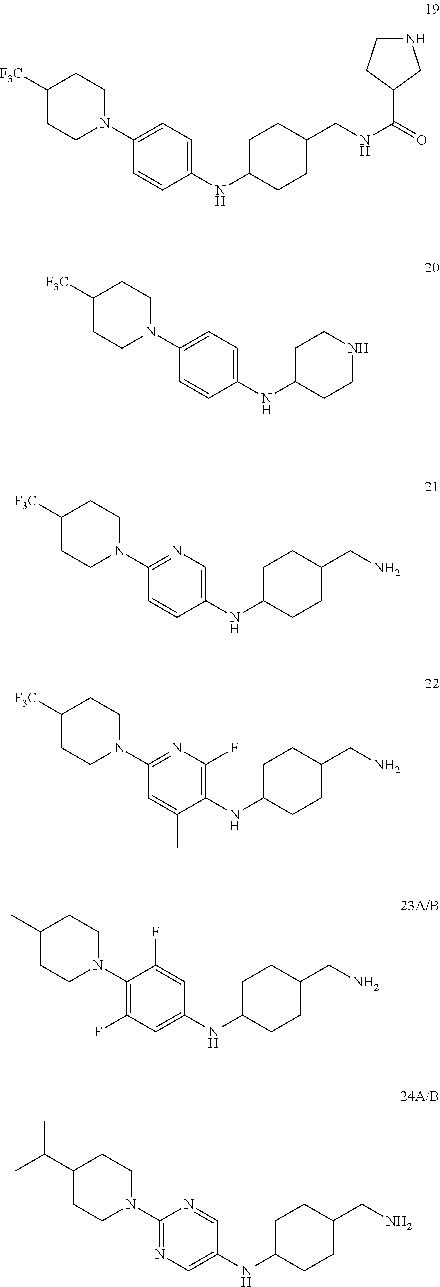 [SMILES: O=C(NCC1CCC(Nc2ccc(N3CCC(C(F)(F)F)CC3)cc2)CC1)C1CCNC1 ; FC(F)(F)C1CCN(c2ccc(NC3CCNCC3)cc2)CC1 ; NCC1CCC(Nc2ccc(N3CCC(C(F)(F)F)CC3)nc2)CC1 ; Cc1cc(N2CCC(C(F)(F)F)CC2)nc(F)c1NC1CCC(CN)CC1 ; CC1CCN(c2c(F)cc(NC3CCC(CN)CC3)cc2F)CC1 ; CC(C)C1CCN(c2ncc(NC3CCC(CN)CC3)cn2)CC1]

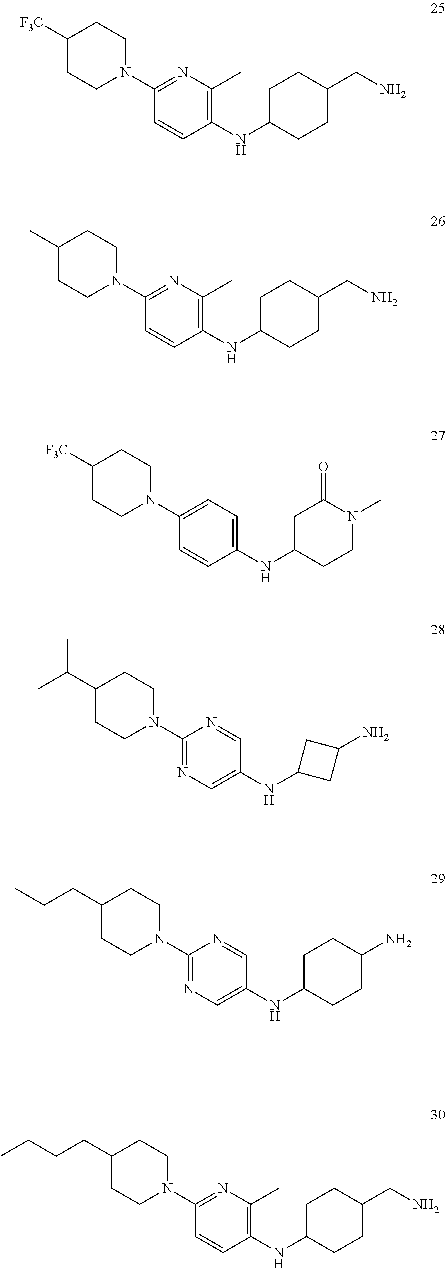

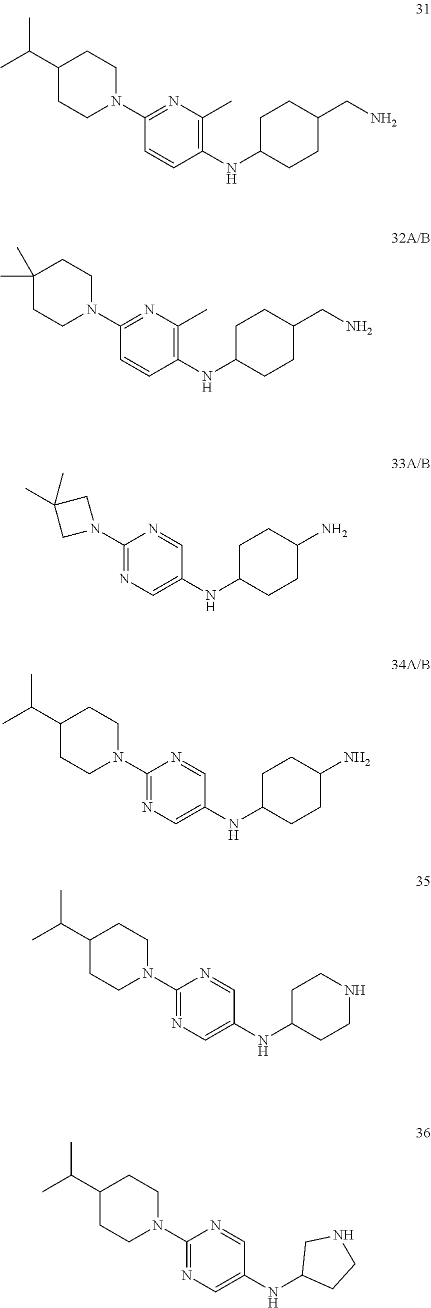 [SMILES: Cc1nc(N2CCC(C(C)C)CC2)ccc1NC1CCC(CN)CC1 ; Cc1nc(N2CCC(C)(C)CC2)ccc1NC1CCC(CN)CC1 ; CC1(C)CN(c2ncc(NC3CCC(N)CC3)cn2)C1 ; CC(C)C1CCN(c2ncc(NC3CCC(N)CC3)cn2)CC1 ; CC(C)C1CCN(c2ncc(NC3CCNCC3)cn2)CC1 ; CC(C)C1CCN(c2ncc(NC3CCNC3)cn2)CC1]

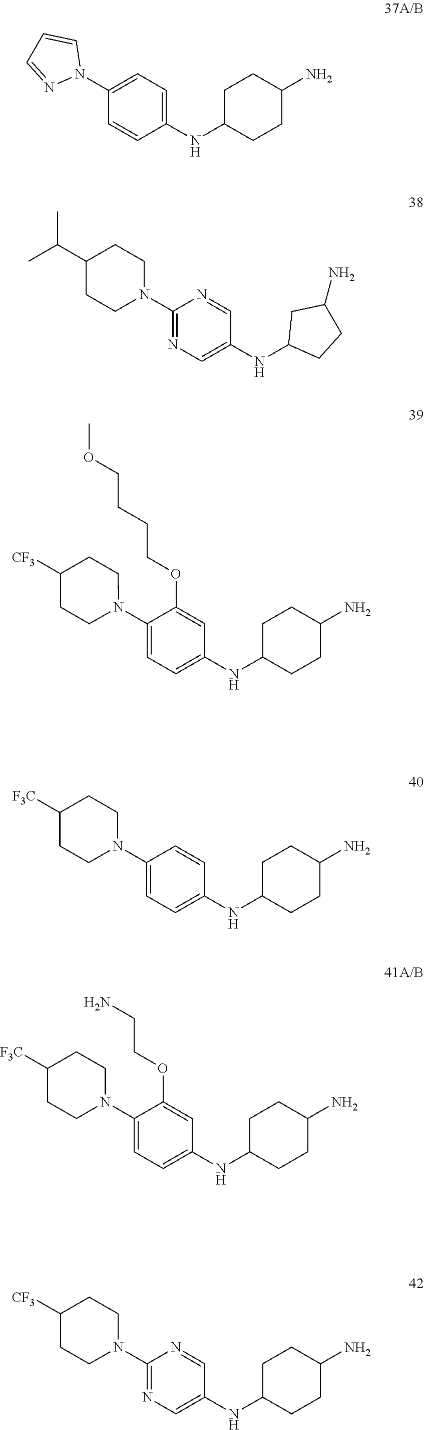 [SMILES: NC1CCC(Nc2ccc(-n3cccn3)cc2)CC1 ; CC(C)C1CCN(c2ncc(NC3CCC(N)C3)cn2)CC1 ; COCCCCOc1cc(NC2CCC(N)CC2)ccc1N1CCC(C(F)(F)F)CC1 ; NC1CCC(Nc2ccc(N3CCC(C(F)(F)F)CC3)cc2)CC1 ; NCCOc1cc(NC2CCC(N)CC2)ccc1N1CCC(C(F)(F)F)CC1 ; NC1CCC(Nc2cnc(N3CCC(C(F)(F)F)CC3)nc2)CC1]

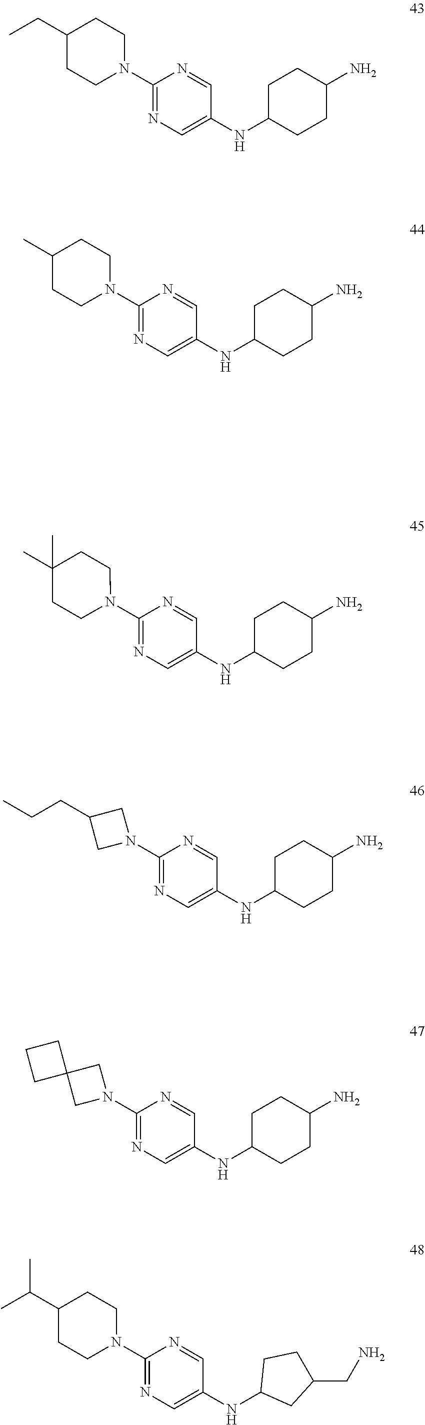 [SMILES: CCC1CCN(c2ncc(NC3CCC(N)CC3)cn2)CC1 ; CC1CCN(c2ncc(NC3CCC(N)CC3)cn2)CC1 ; CC1(C)CCN(c2ncc(NC3CCC(N)CC3)cn2)CC1 ; CCCC1CN(c2ncc(NC3CCC(N)CC3)cn2)C1 ; NC1CCC(Nc2cnc(N3CC4(CCC4)C3)nc2)CC1 ; CC(C)C1CCN(c2ncc(NC3CCC(CN)C3)cn2)CC1]

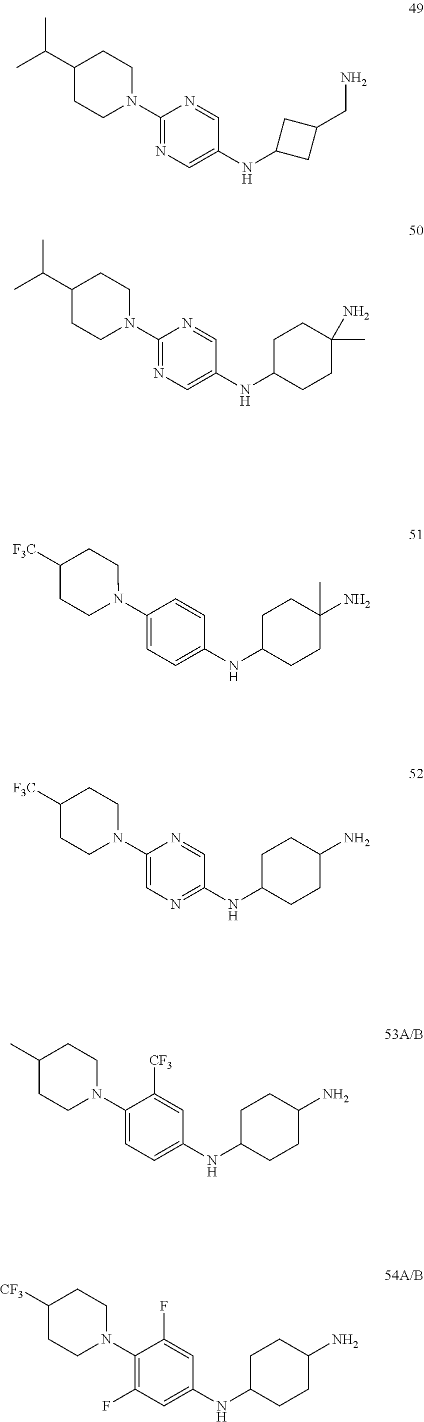

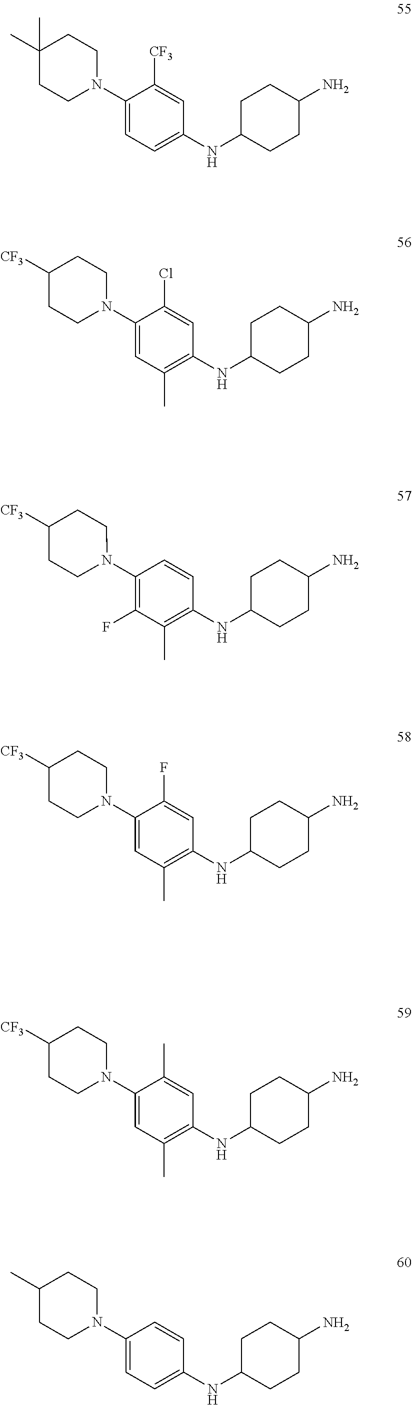 [SMILES: CC1(C)CCN(c2ccc(NC3CCC(N)CC3)cc2C(F)(F)F)CC1 ; Cc1cc(N2CCC(C(F)(F)F)CC2)c(Cl)cc1NC1CCC(N)CC1 ; Cc1c(NC2CCC(N)CC2)ccc(N2CCC(C(F)(F)F)CC2)c1F ; Cc1cc(N2CCC(C(F)(F)F)CC2)c(F)cc1NC1CCC(N)CC1 ; Cc1cc(N2CCC(C(F)(F)F)CC2)c(C)cc1NC1CCC(N)CC1 ; CC1CCN(c2ccc(NC3CCC(N)CC3)cc2)CC1]

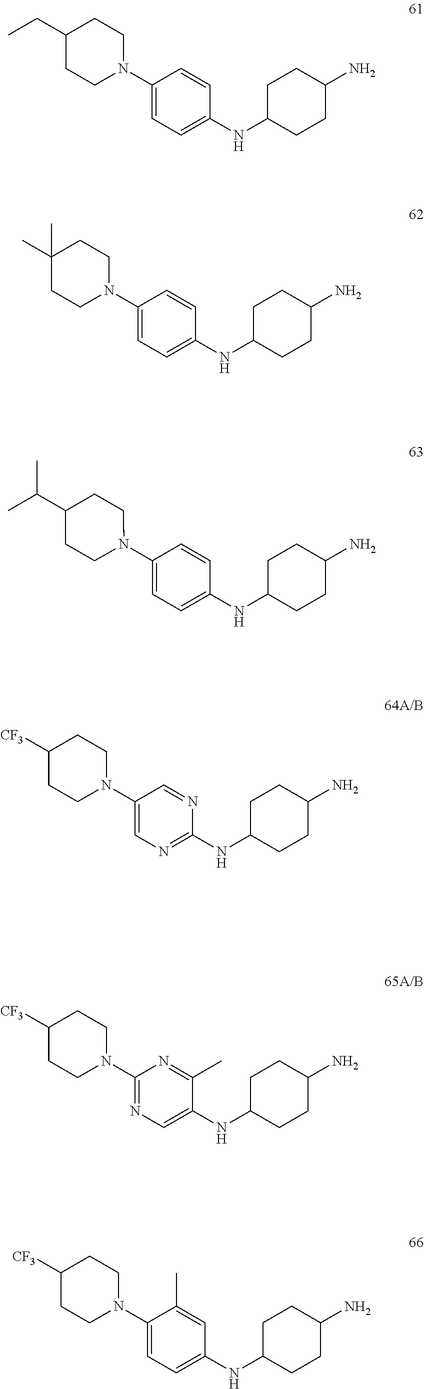 [SMILES: CCC1CCN(c2ccc(NC3CCC(N)CC3)cc2)CC1 ; CC1(C)CCN(c2ccc(NC3CCC(N)CC3)cc2)CC1 ; CC(C)C1CCN(c2ccc(NC3CCC(N)CC3)cc2)CC1 ; NC1CCC(Nc2ncc(N3CCC(C(F)(F)F)CC3)cn2)CC1 ; Cc1nc(N2CCC(C(F)(F)F)CC2)ncc1NC1CCC(N)CC1 ; Cc1cc(NC2CCC(N)CC2)ccc1N1CCC(C(F)(F)F)CC1]

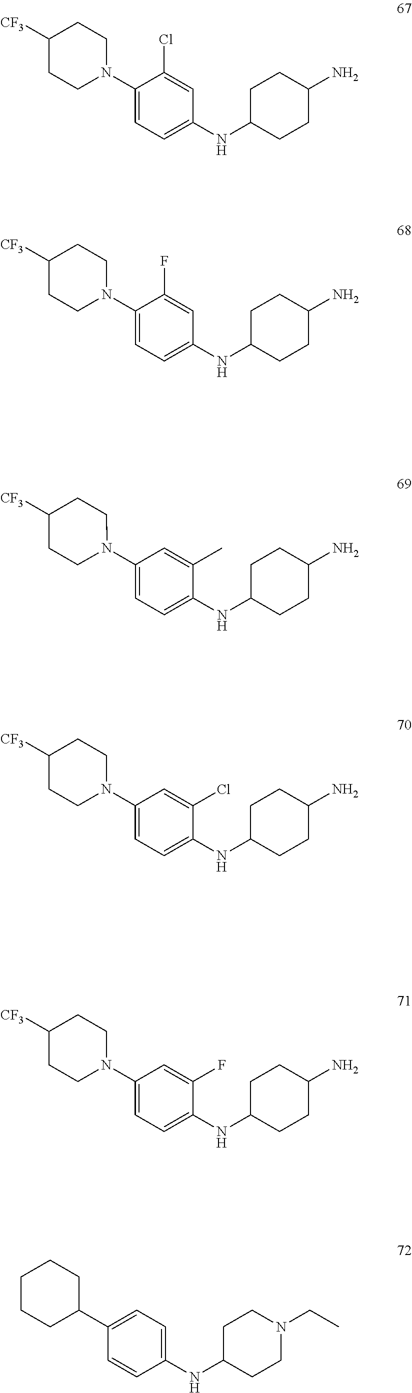 [SMILES: NC1CCC(Nc2ccc(N3CCC(C(F)(F)F)CC3)c(Cl)c2)CC1 ; NC1CCC(Nc2ccc(N3CCC(C(F)(F)F)CC3)c(F)c2)CC1 ; Cc1cc(N2CCC(C(F)(F)F)CC2)ccc1NC1CCC(N)CC1 ; NC1CCC(Nc2ccc(N3CCC(C(F)(F)F)CC3)cc2Cl)CC1 ; NC1CCC(Nc2ccc(N3CCC(C(F)(F)F)CC3)cc2F)CC1 ; CCN1CCC(Nc2ccc(C3CCCCC3)cc2)CC1]

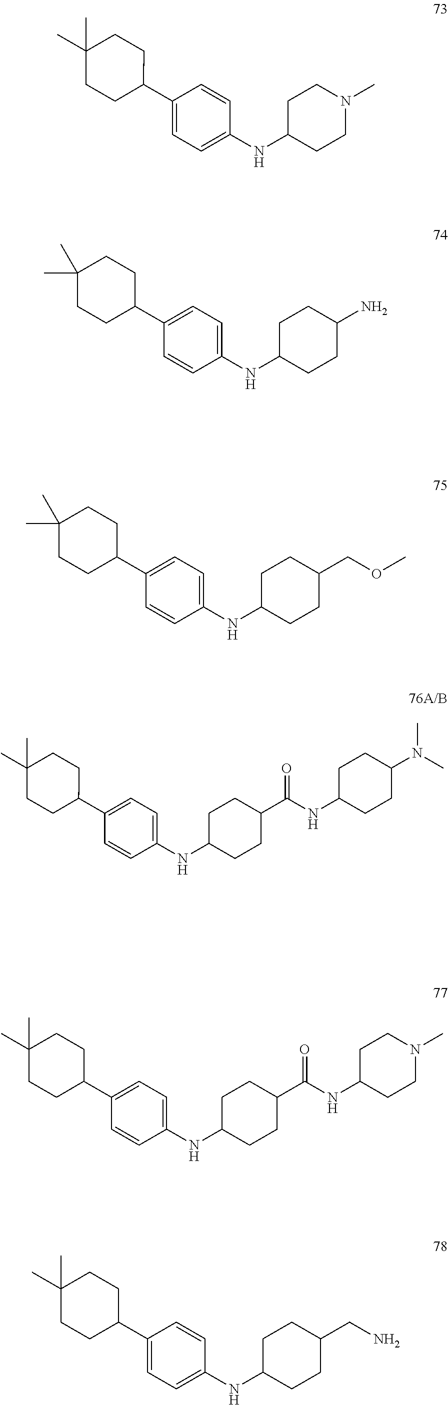

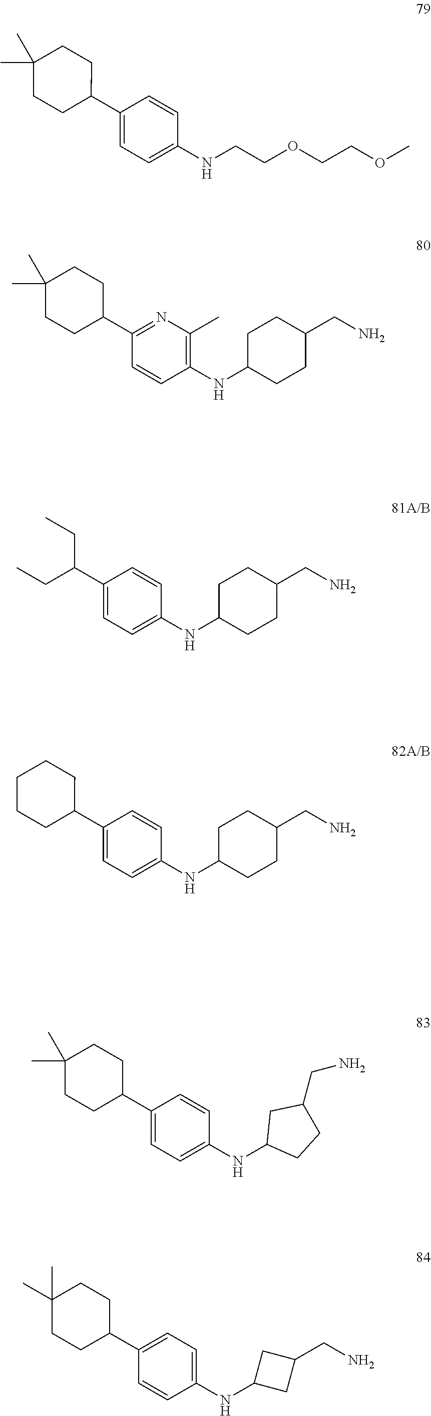

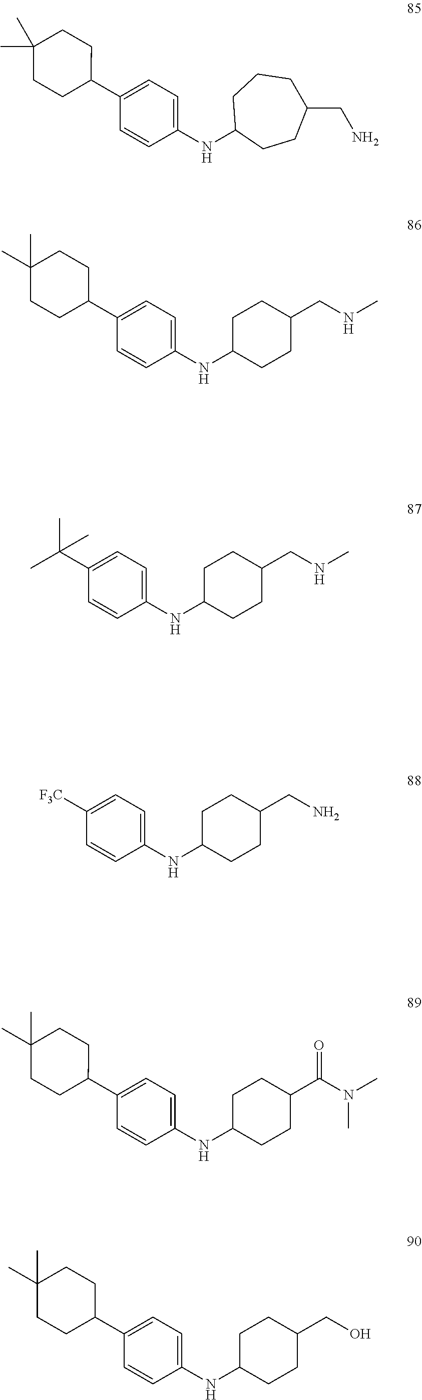

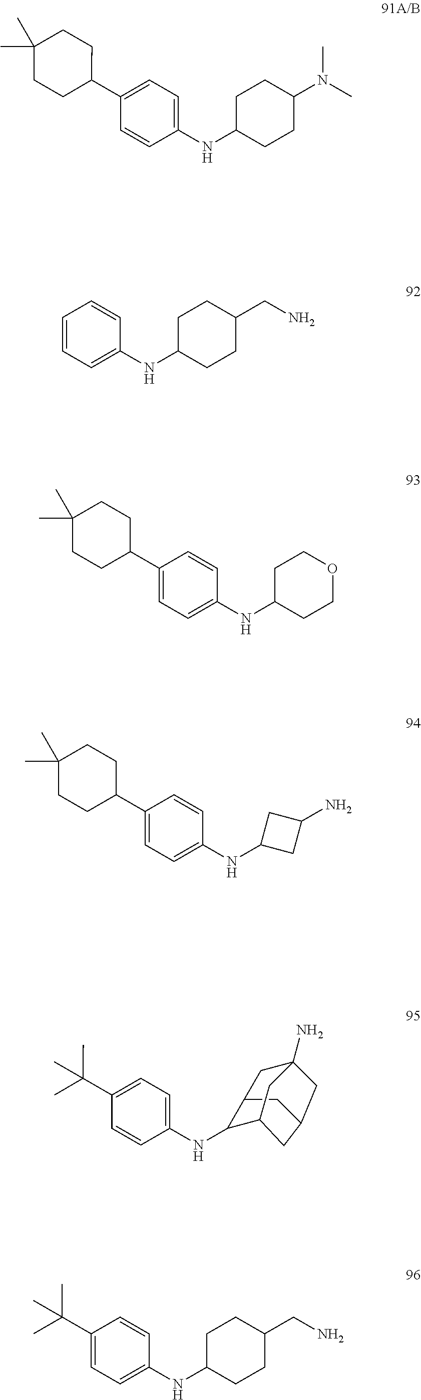

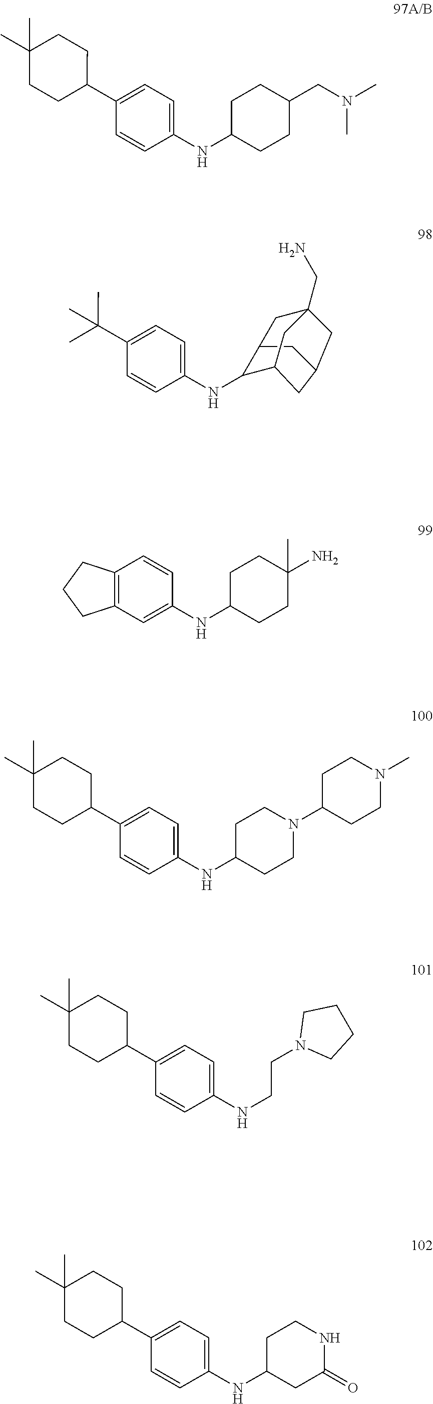

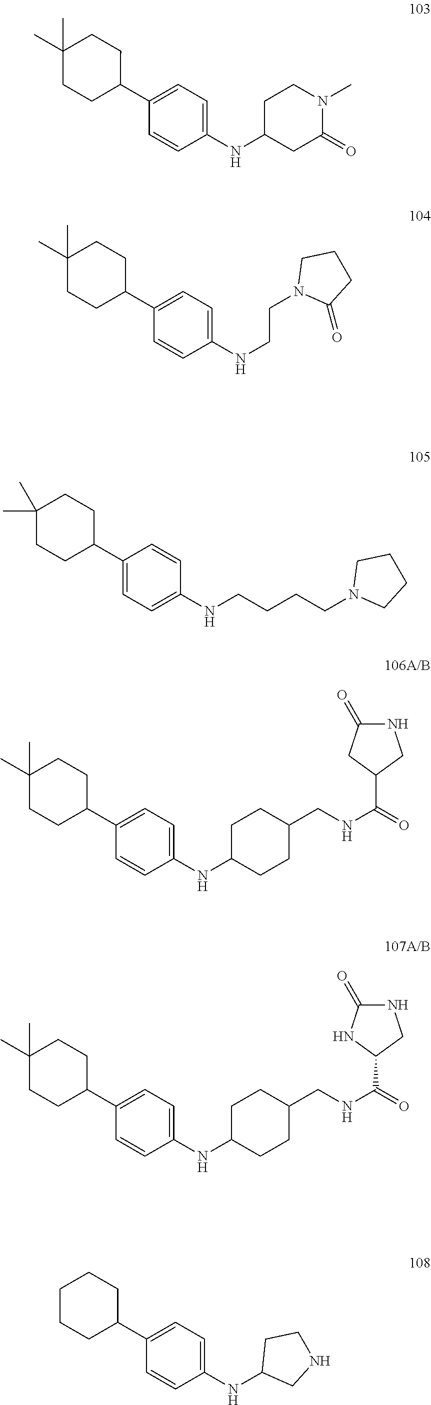 [SMILES: CN1CCC(Nc2ccc(C3CCC(C)(C)CC3)cc2)CC1=O ; CC1(C)CCC(c2ccc(NCCN3CCCC3=O)cc2)CC1 ; CC1(C)CCC(c2ccc(NCCCCN3CCCC3)cc2)CC1 ; CC1(C)CCC(c2ccc(NC3CCC(CNC(=O)C4CNC(=O)C4)CC3)cc2)CC1 ; CC1(C)CCC(c2ccc(NC3CCC(CNC(=O)[C@H]4CNC(=O)N4)CC3)cc2)CC1 ; c1cc(C2CCCCC2)ccc1NC1CCNC1]

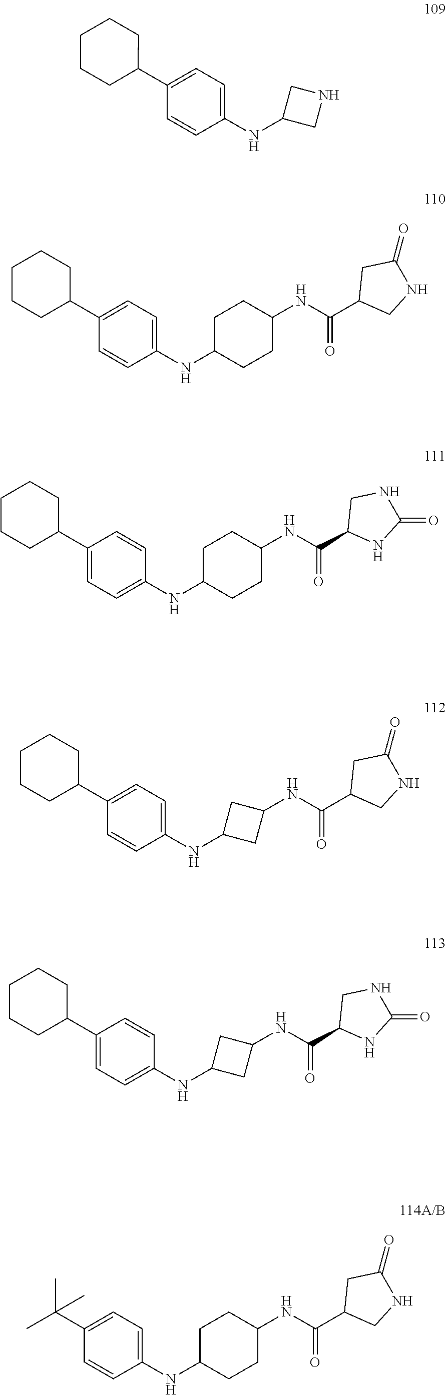 [SMILES: c1cc(C2CCCCC2)ccc1NC1CNC1 ; O=C1CC(C(=O)NC2CCC(Nc3ccc(C4CCCCC4)cc3)CC2)CN1 ; O=C1NC[C@H](C(=O)NC2CCC(Nc3ccc(C4CCCCC4)cc3)CC2)N1 ; O=C1CC(C(=O)NC2CC(Nc3ccc(C4CCCCC4)cc3)C2)CN1 ; O=C1NC[C@H](C(=O)NC2CC(Nc3ccc(C4CCCCC4)cc3)C2)N1 ; CC(C)(C)c1ccc(NC2CCC(NC(=O)C3CNC(=O)C3)CC2)cc1]

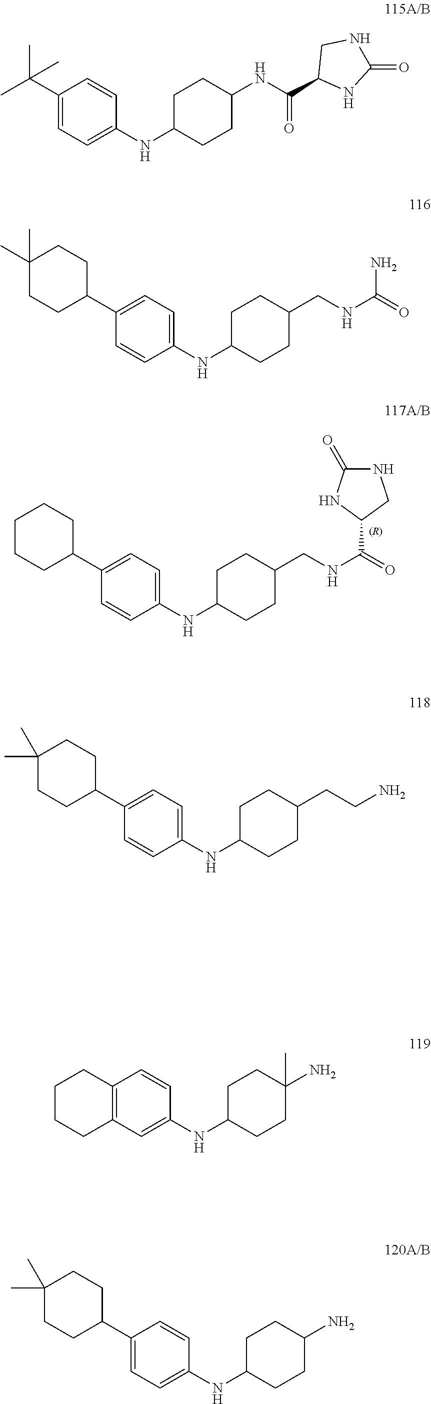

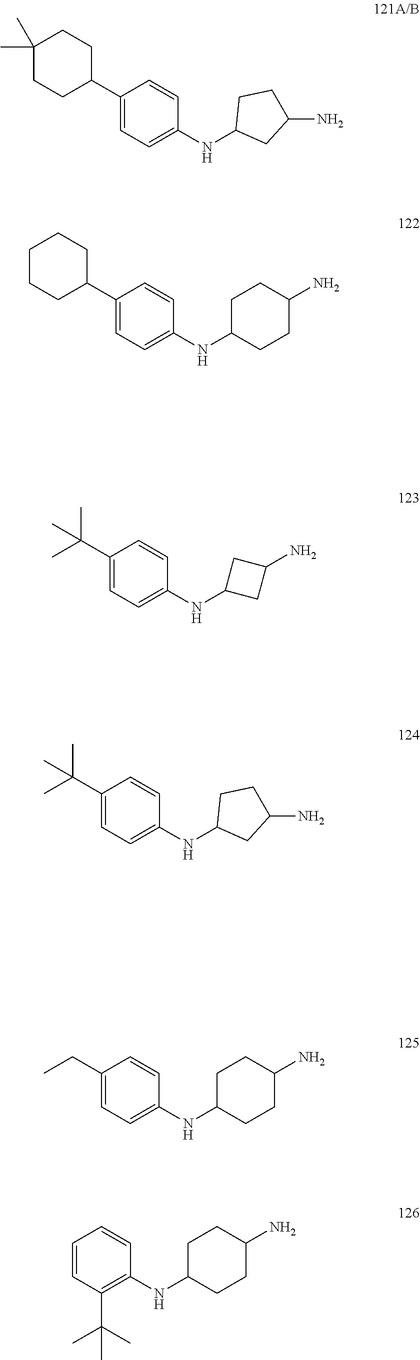 [SMILES: CC1(C)CCC(c2ccc(NC3CCC(N)C3)cc2)CC1 ; NC1CCC(Nc2ccc(C3CCCCC3)cc2)CC1 ; CC(C)(C)c1ccc(NC2CC(N)C2)cc1 ; CC(C)(C)c1ccc(NC2CCC(N)C2)cc1 ; CCc1ccc(NC2CCC(N)CC2)cc1 ; CC(C)(C)c1ccccc1NC1CCC(N)CC1]

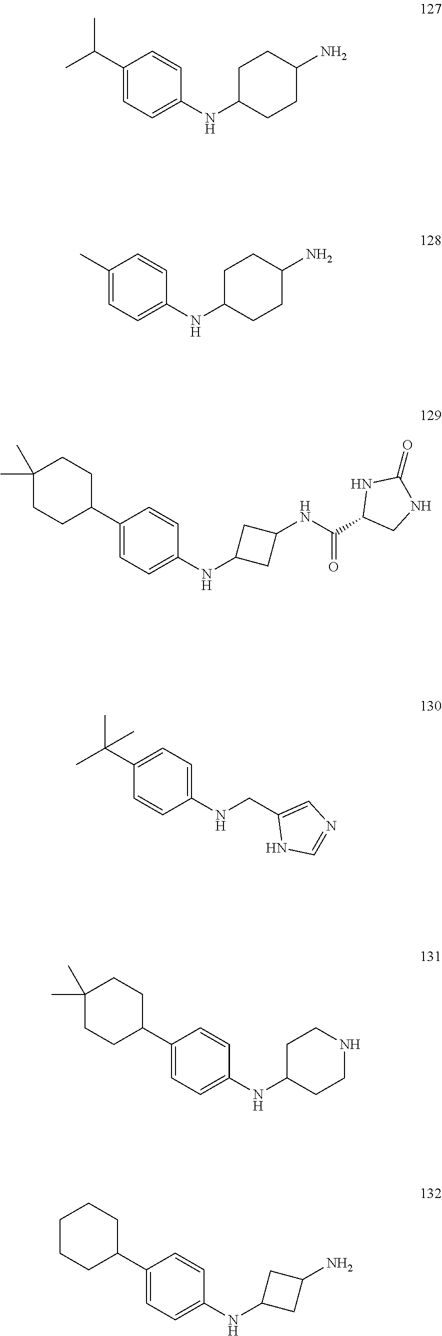 [SMILES: CC(C)c1ccc(NC2CCC(N)CC2)cc1 ; Cc1ccc(NC2CCC(N)CC2)cc1 ; CC1(C)CCC(c2ccc(NC3CC(NC(=O)[C@H]4CNC(=O)N4)C3)cc2)CC1 ; CC(C)(C)c1ccc(NCc2cnc[nH]2)cc1 ; CC1(C)CCC(c2ccc(NC3CCNCC3)cc2)CC1 ; NC1CC(Nc2ccc(C3CCCCC3)cc2)C1]

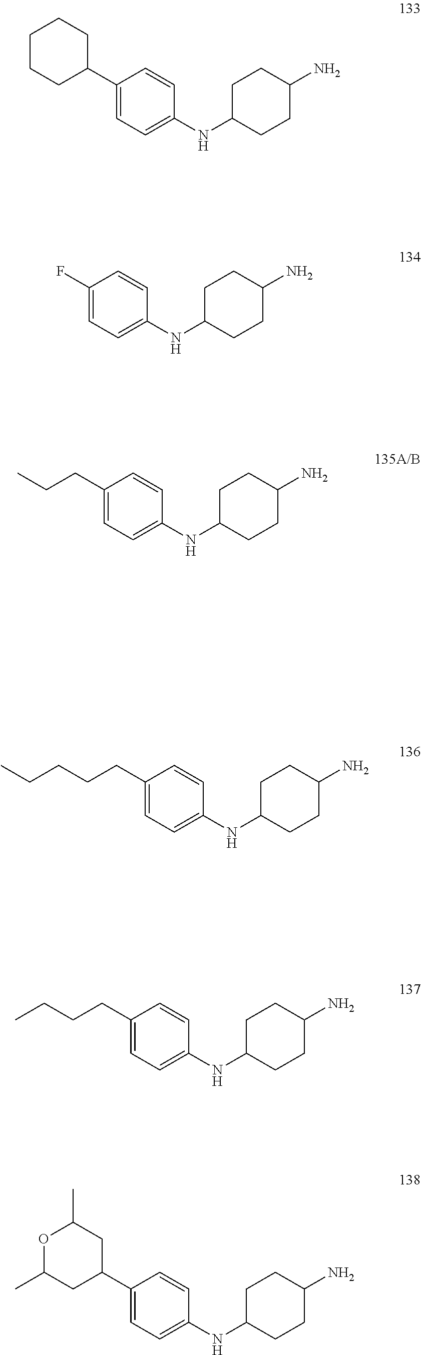 [SMILES: NC1CCC(Nc2ccc(C3CCCCC3)cc2)CC1 ; NC1CCC(Nc2ccc(F)cc2)CC1 ; CCCc1ccc(NC2CCC(N)CC2)cc1 ; CCCCCc1ccc(NC2CCC(N)CC2)cc1 ; CCCCc1ccc(NC2CCC(N)CC2)cc1 ; CC1CC(c2ccc(NC3CCC(N)CC3)cc2)CC(C)O1]

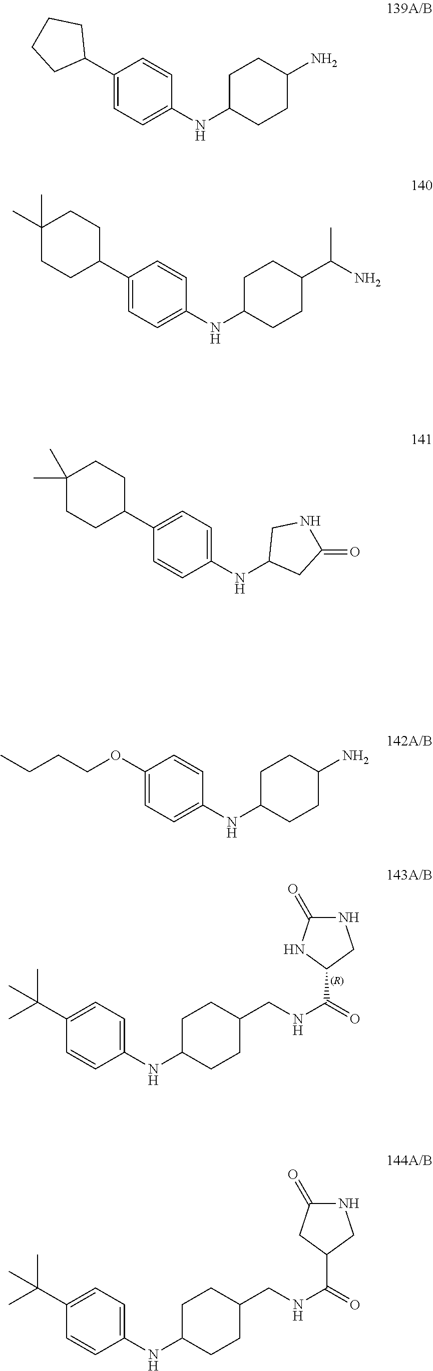

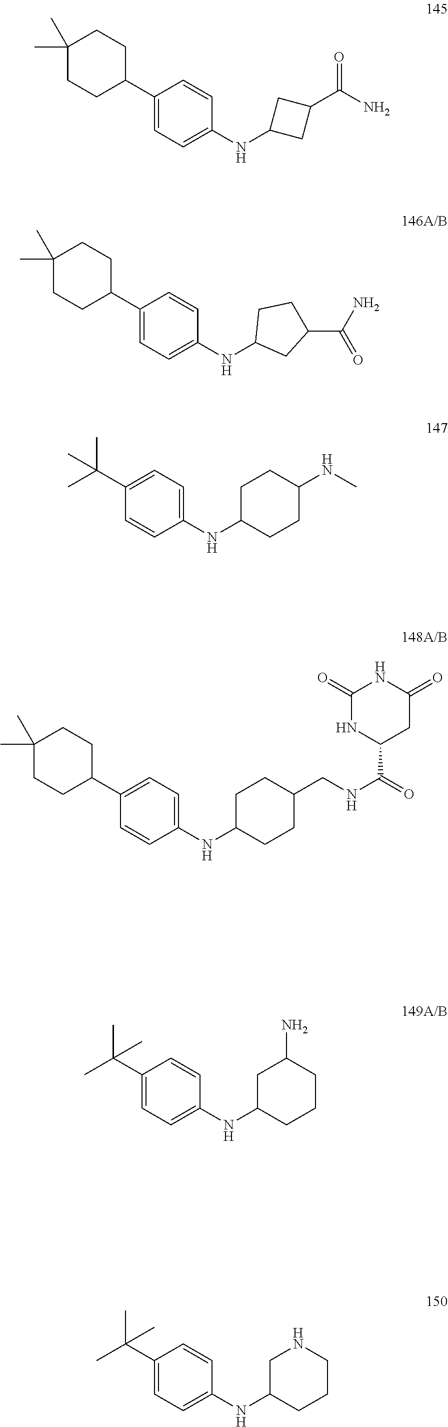

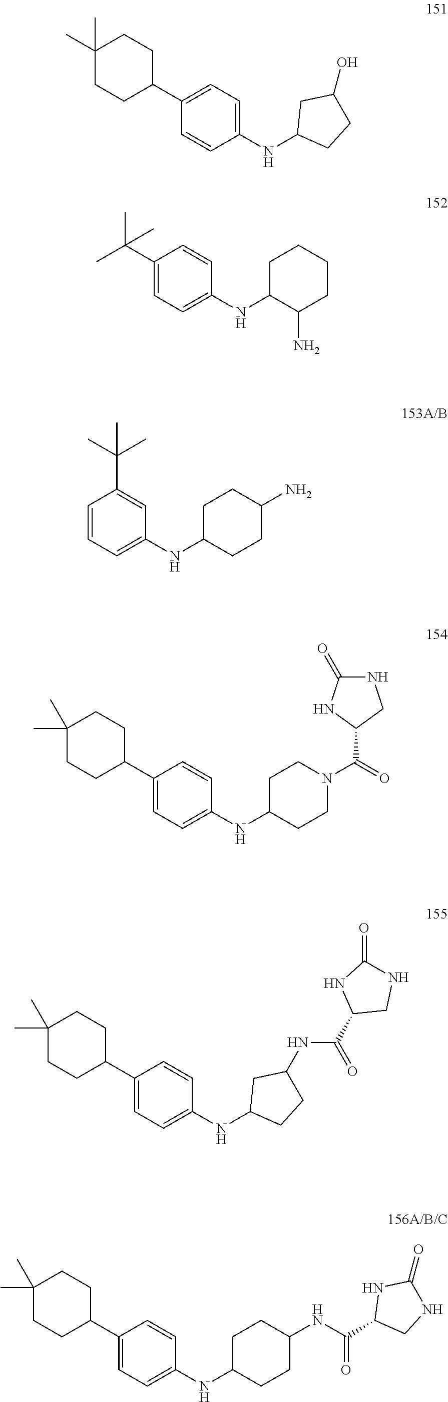 [SMILES: CC1(C)CCC(c2ccc(NC3CCC(O)C3)cc2)CC1 ; CC(C)(C)c1ccc(NC2CCCCC2N)cc1 ; CC(C)(C)c1cccc(NC2CCC(N)CC2)c1 ; CC1(C)CCC(c2ccc(NC3CCN(C(=O)[C@H]4CNC(=O)N4)CC3)cc2)CC1 ; CC1(C)CCC(c2ccc(NC3CCC(NC(=O)[C@H]4CNC(=O)N4)C3)cc2)CC1 ; CC1(C)CCC(c2ccc(NC3CCC(NC(=O)[C@H]4CNC(=O)N4)CC3)cc2)CC1]

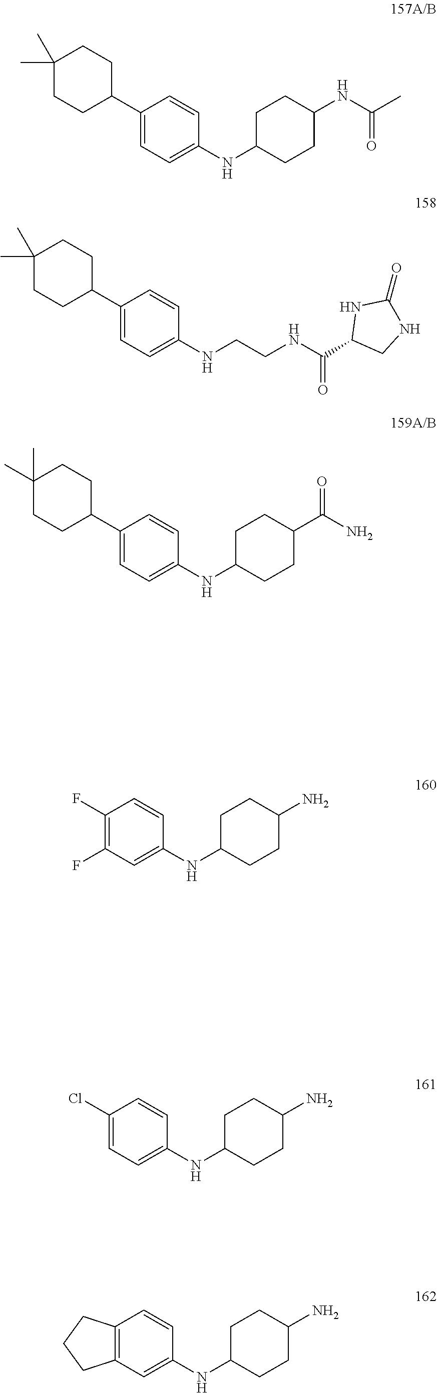

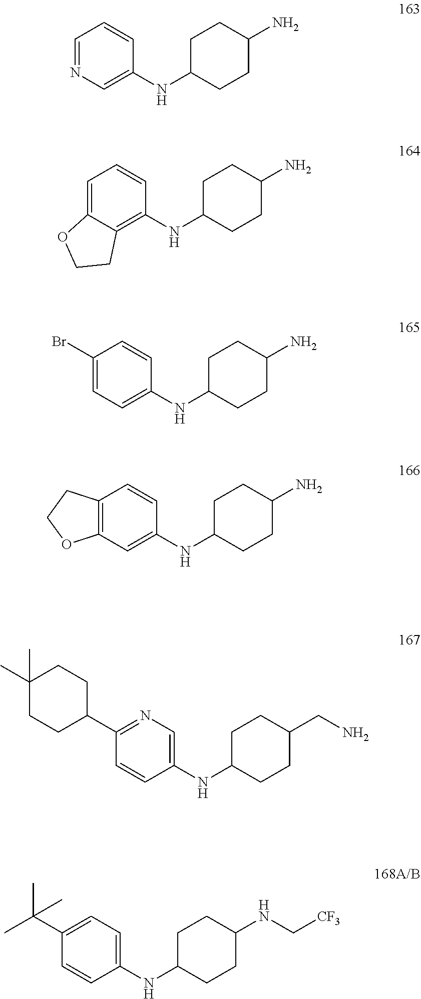 [SMILES: NC1CCC(Nc2cccnc2)CC1 ; NC1CCC(Nc2cccc3c2CCO3)CC1 ; NC1CCC(Nc2ccc(Br)cc2)CC1 ; NC1CCC(Nc2ccc3c(c2)OCC3)CC1 ; CC1(C)CCC(c2ccc(NC3CCC(CN)CC3)cn2)CC1 ; CC(C)(C)c1ccc(NC2CCC(NCC(F)(F)F)CC2)cc1]

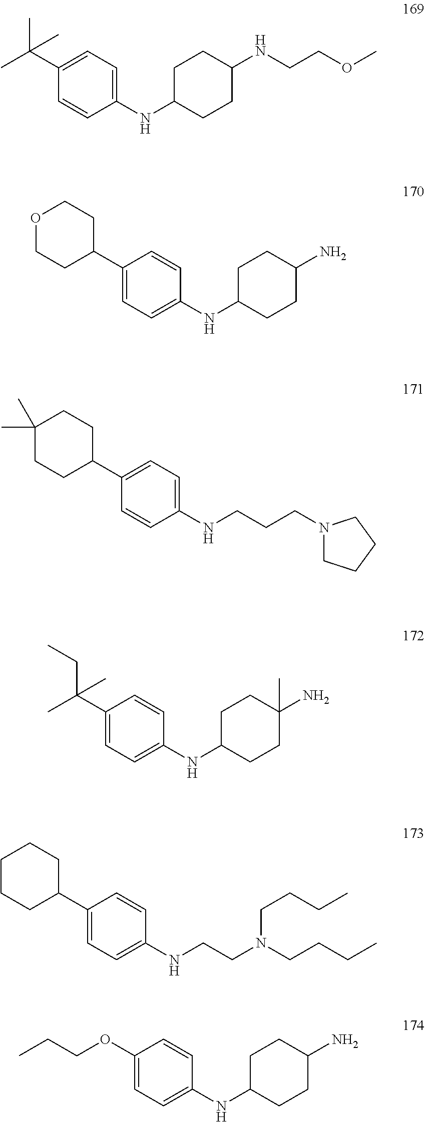

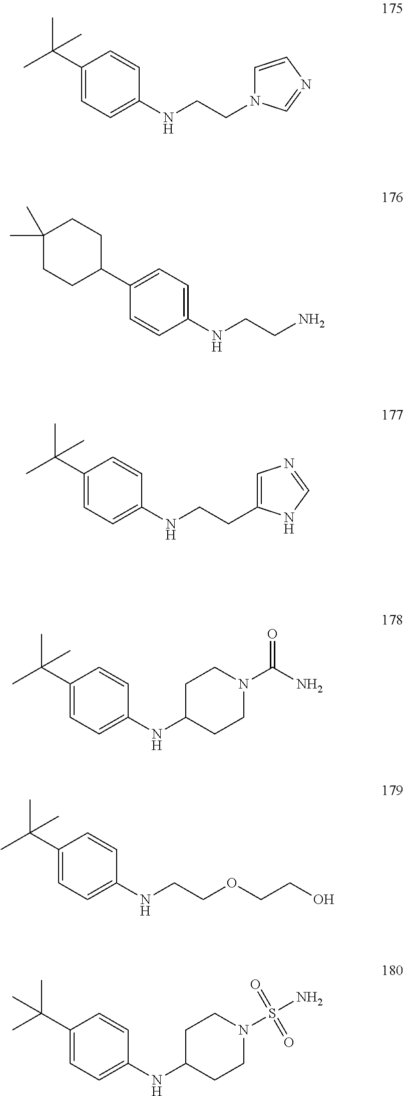 [SMILES: CC(C)(C)c1ccc(NCCn2ccnc2)cc1 ; CC1(C)CCC(c2ccc(NCCN)cc2)CC1 ; CC(C)(C)c1ccc(NCCc2cnc[nH]2)cc1 ; CC(C)(C)c1ccc(NC2CCN(C(N)=O)CC2)cc1 ; CC(C)(C)c1ccc(NCCOCCO)cc1 ; CC(C)(C)c1ccc(NC2CCN(S(N)(=O)=O)CC2)cc1]

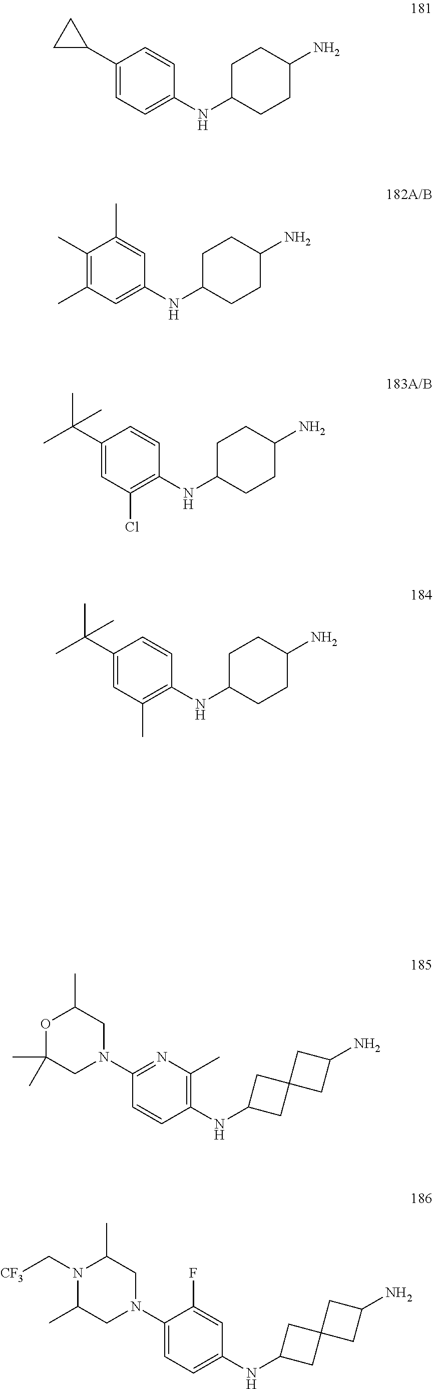 [SMILES: NC1CCC(Nc2ccc(C3CC3)cc2)CC1 ; Cc1cc(NC2CCC(N)CC2)cc(C)c1C ; CC(C)(C)c1ccc(NC2CCC(N)CC2)c(Cl)c1 ; Cc1cc(C(C)(C)C)ccc1NC1CCC(N)CC1 ; Cc1nc(N2CC(C)OC(C)(C)C2)ccc1NC1CC2(CC(N)C2)C1 ; CC1CN(c2ccc(NC3CC4(CC(N)C4)C3)cc2F)CC(C)N1CC(F)(F)F]

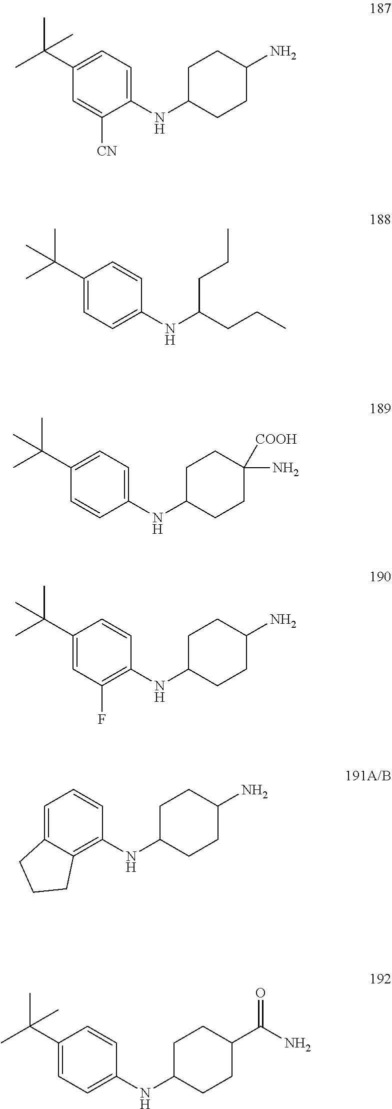

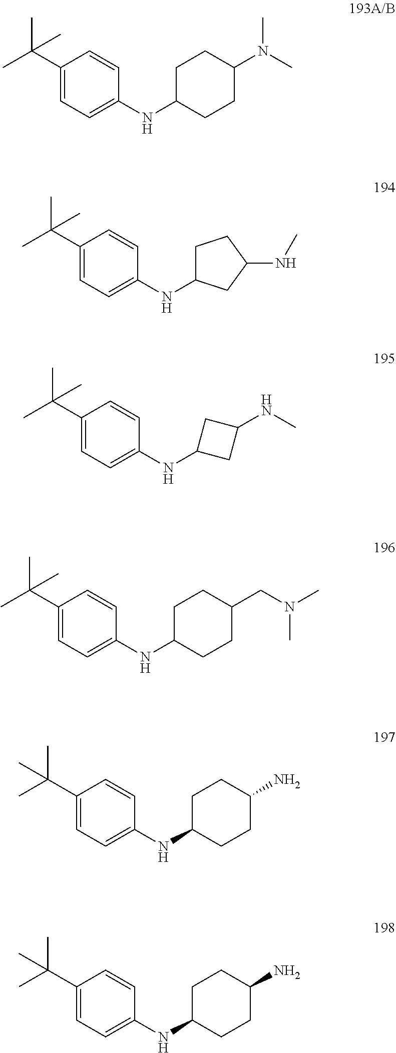

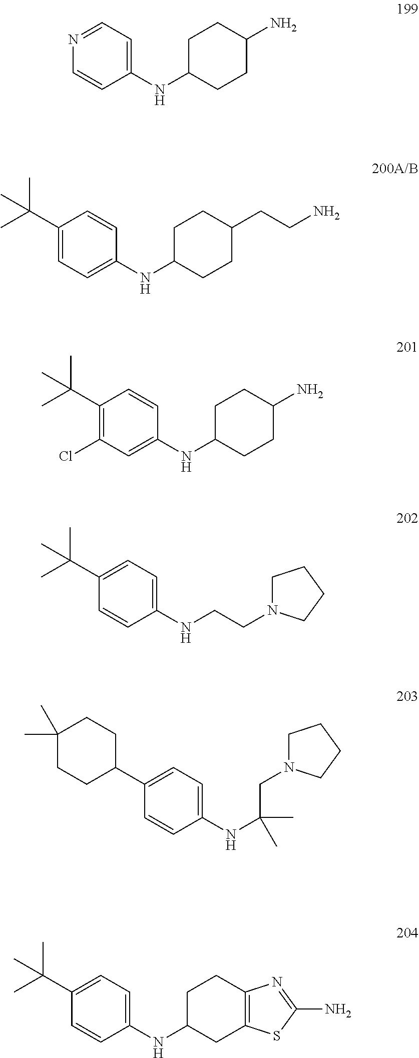 [SMILES: NC1CCC(Nc2ccncc2)CC1 ; CC(C)(C)c1ccc(NC2CCC(CCN)CC2)cc1 ; CC(C)(C)c1ccc(NC2CCC(N)CC2)cc1Cl ; CC(C)(C)c1ccc(NCCN2CCCC2)cc1 ; CC1(C)CCC(c2ccc(NC(C)(C)CN3CCCC3)cc2)CC1 ; CC(C)(C)c1ccc(NC2CCc3nc(N)sc3C2)cc1]

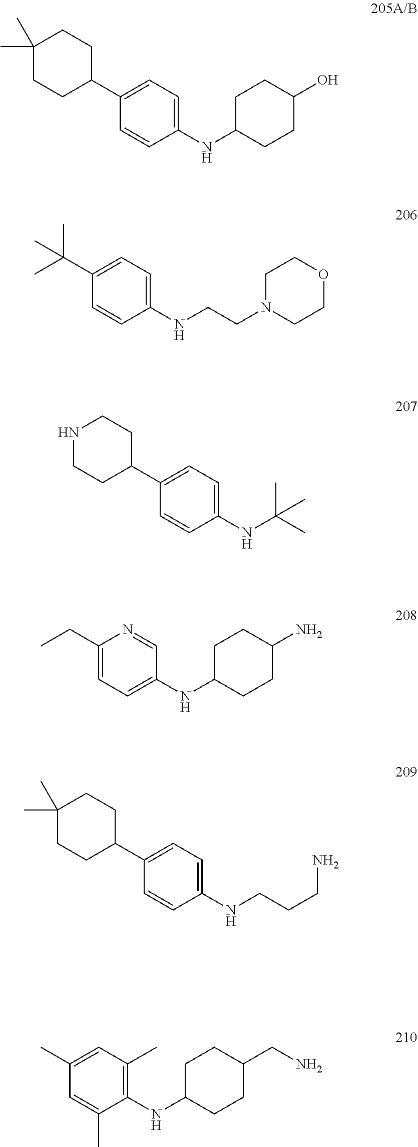

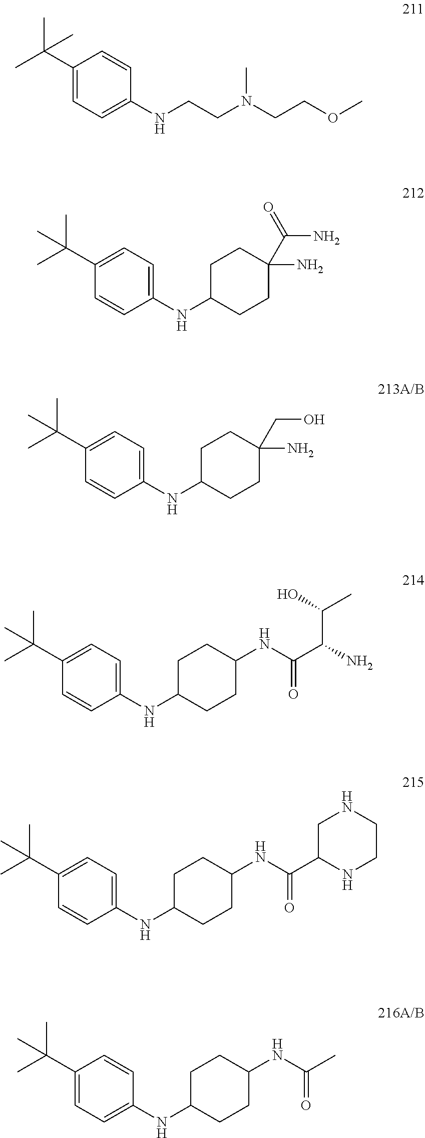

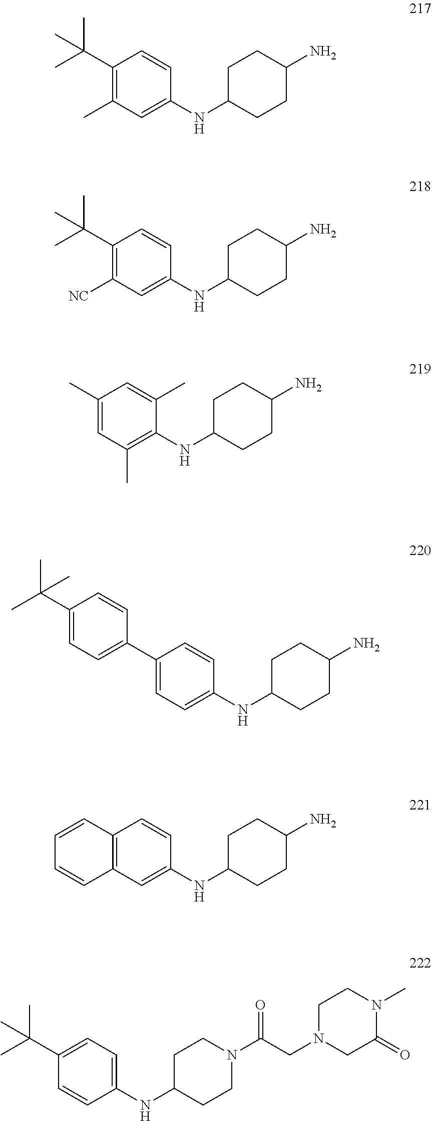 [SMILES: Cc1cc(NC2CCC(N)CC2)ccc1C(C)(C)C ; CC(C)(C)c1ccc(NC2CCC(N)CC2)cc1C#N ; Cc1cc(C)c(NC2CCC(N)CC2)c(C)c1 ; CC(C)(C)c1ccc(-c2ccc(NC3CCC(N)CC3)cc2)cc1 ; NC1CCC(Nc2ccc3ccccc3c2)CC1 ; CN1CCN(CC(=O)N2CCC(Nc3ccc(C(C)(C)C)cc3)CC2)CC1=O]

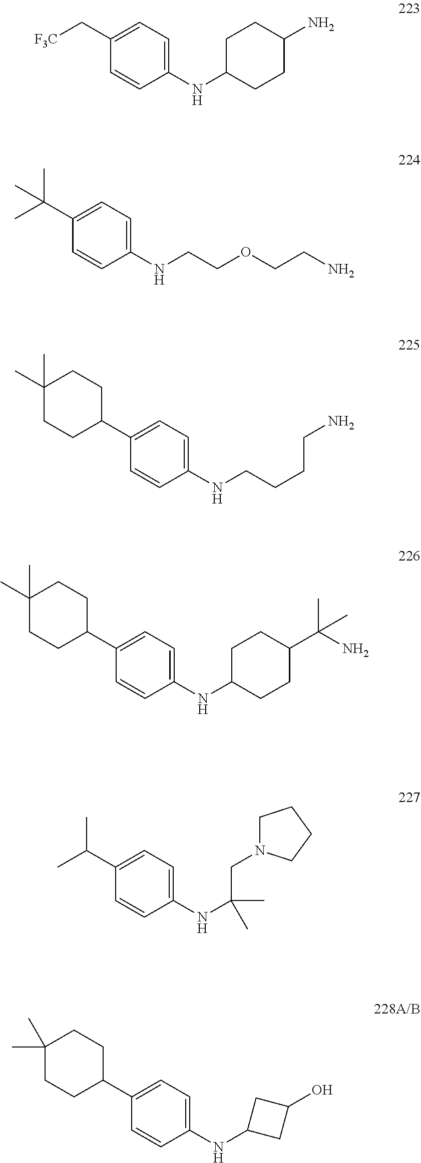

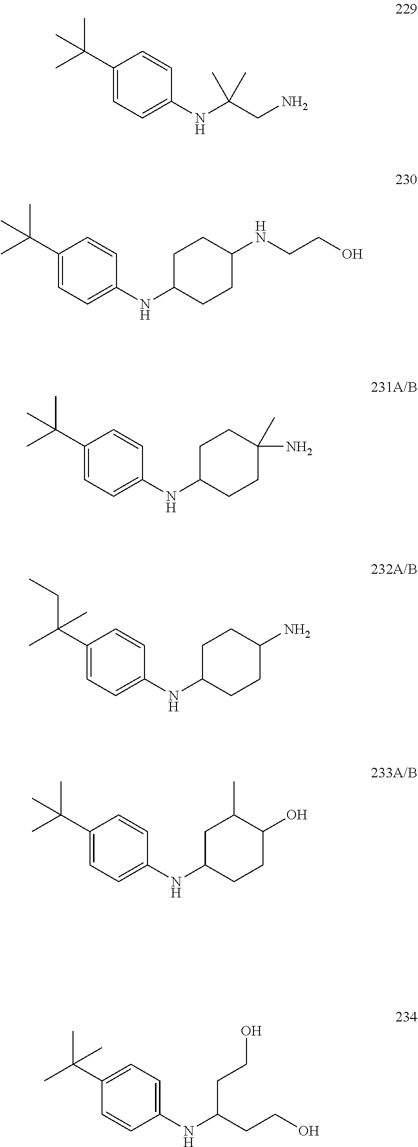 [SMILES: CC(C)(CN)Nc1ccc(C(C)(C)C)cc1 ; CC(C)(C)c1ccc(NC2CCC(NCCO)CC2)cc1 ; CC1(N)CCC(Nc2ccc(C(C)(C)C)cc2)CC1 ; CCC(C)(C)c1ccc(NC2CCC(N)CC2)cc1 ; CC1CC(Nc2ccc(C(C)(C)C)cc2)CCC1O ; CC(C)(C)c1ccc(NC(CCO)CCO)cc1]

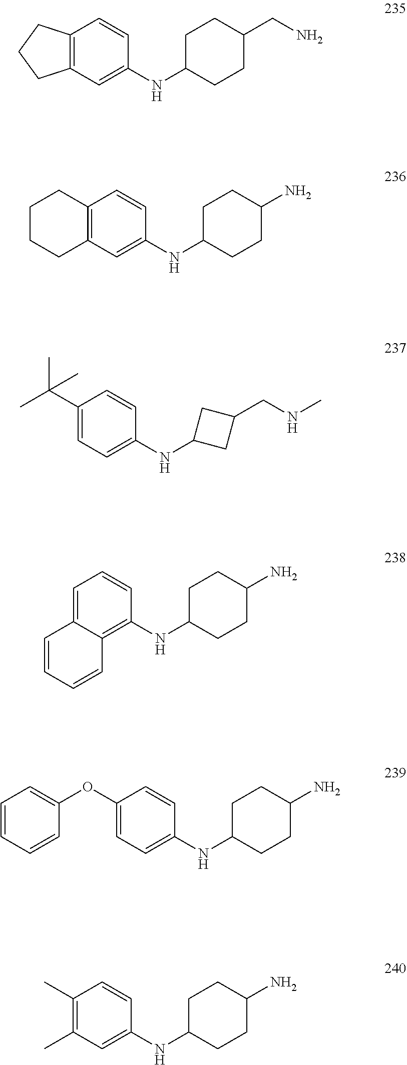

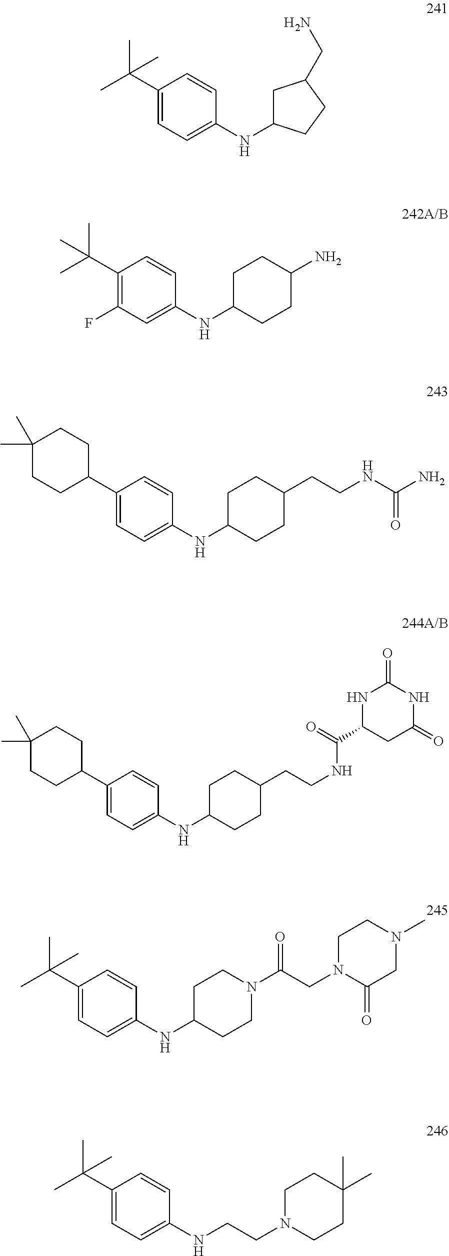

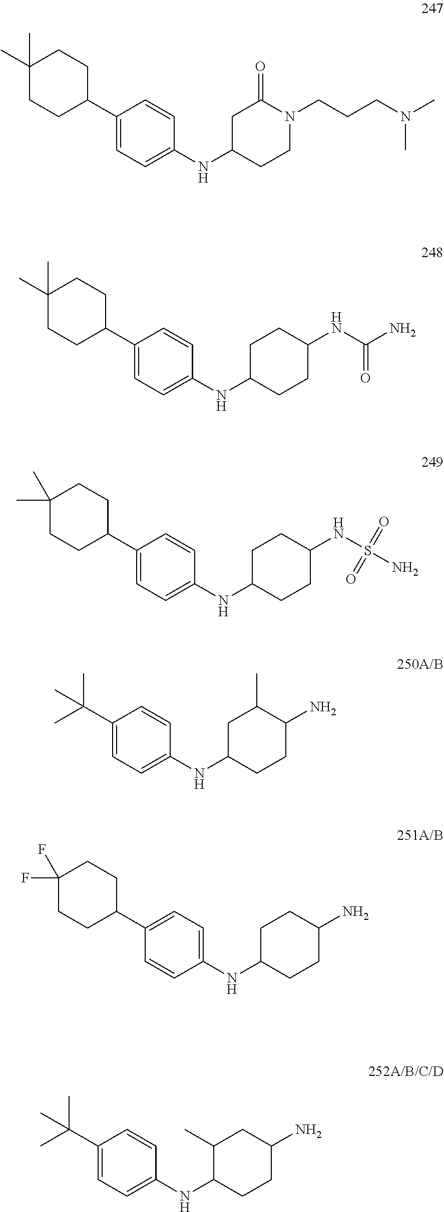

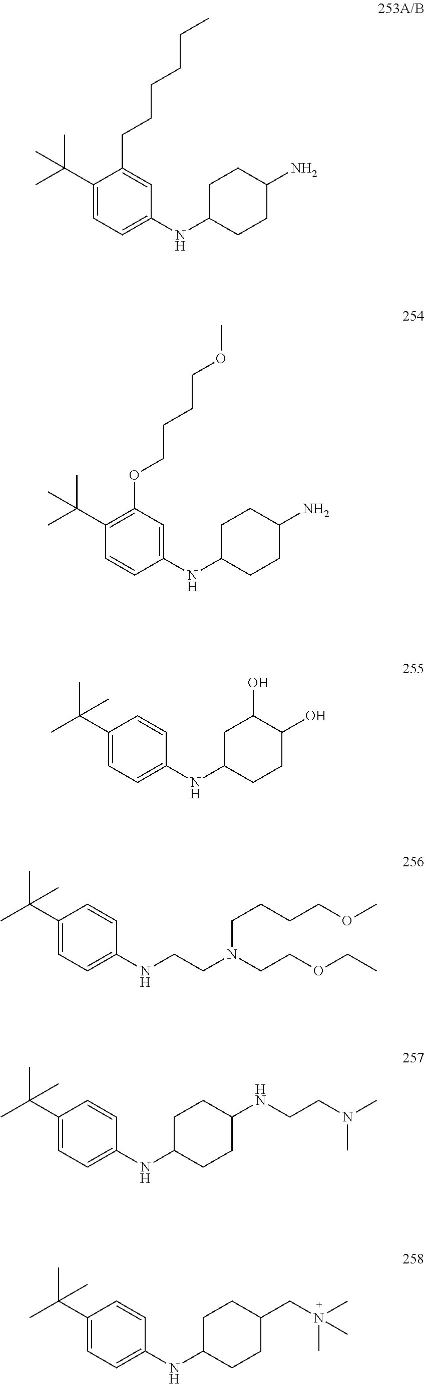

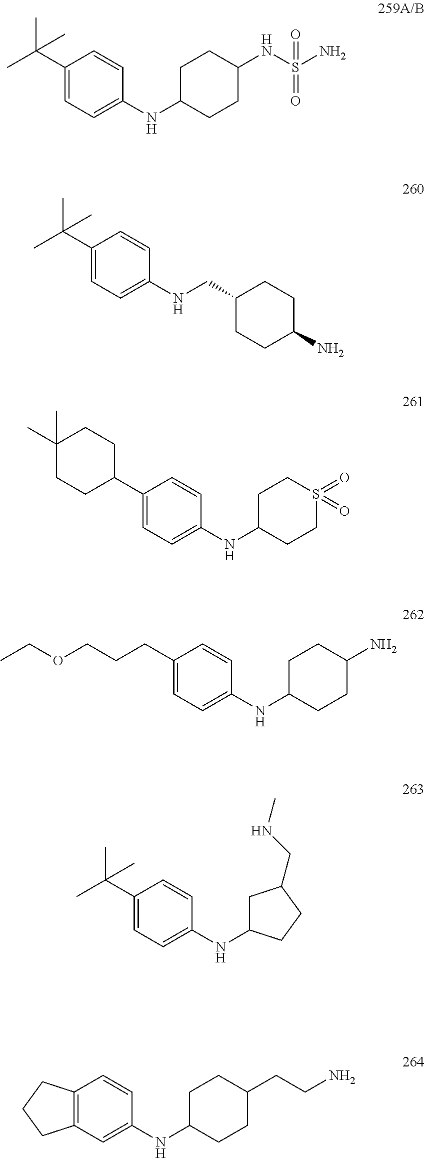 [SMILES: CC(C)(C)c1ccc(NC2CCC(NS(N)(=O)=O)CC2)cc1 ; CC(C)(C)c1ccc(NC[C@H]2CC[C@H](N)CC2)cc1 ; CC1(C)CCC(c2ccc(NC3CCS(=O)(=O)CC3)cc2)CC1 ; CCOCCCc1ccc(NC2CCC(N)CC2)cc1 ; CNCC1CCC(Nc2ccc(C(C)(C)C)cc2)C1 ; NCCC1CCC(Nc2ccc3c(c2)CCC3)CC1]

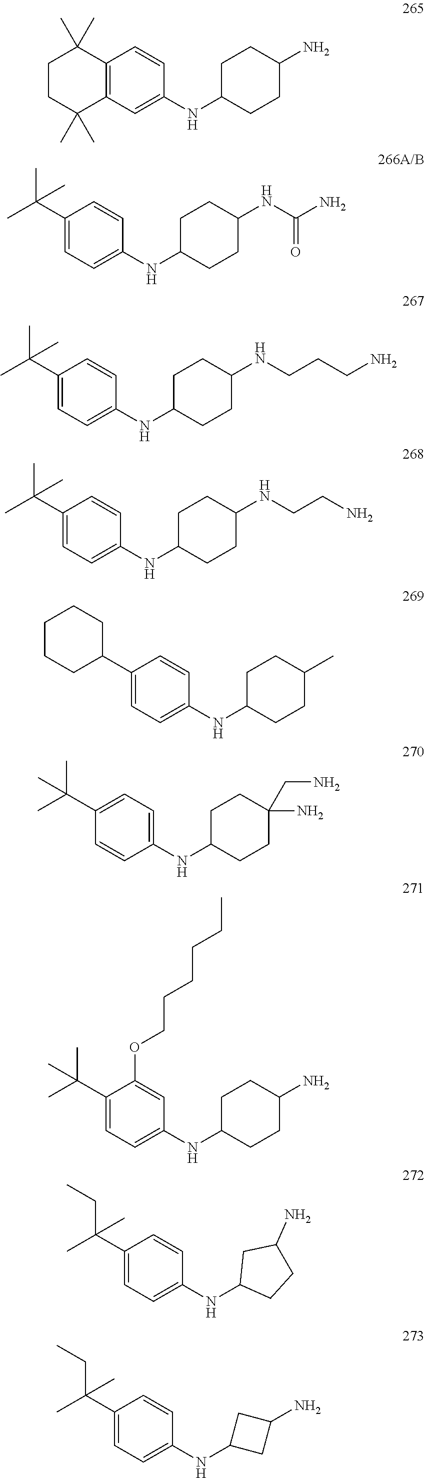

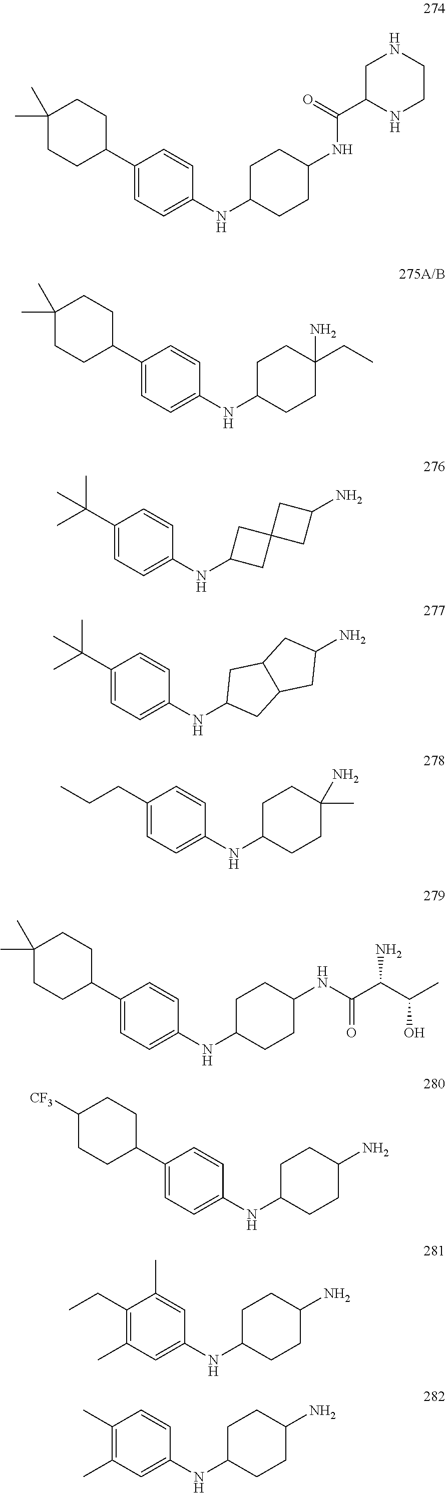

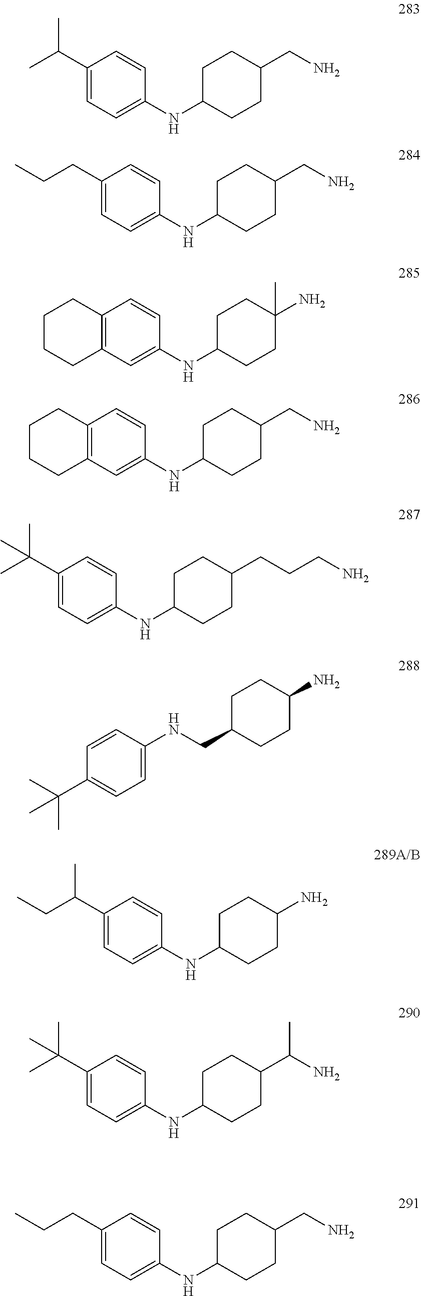

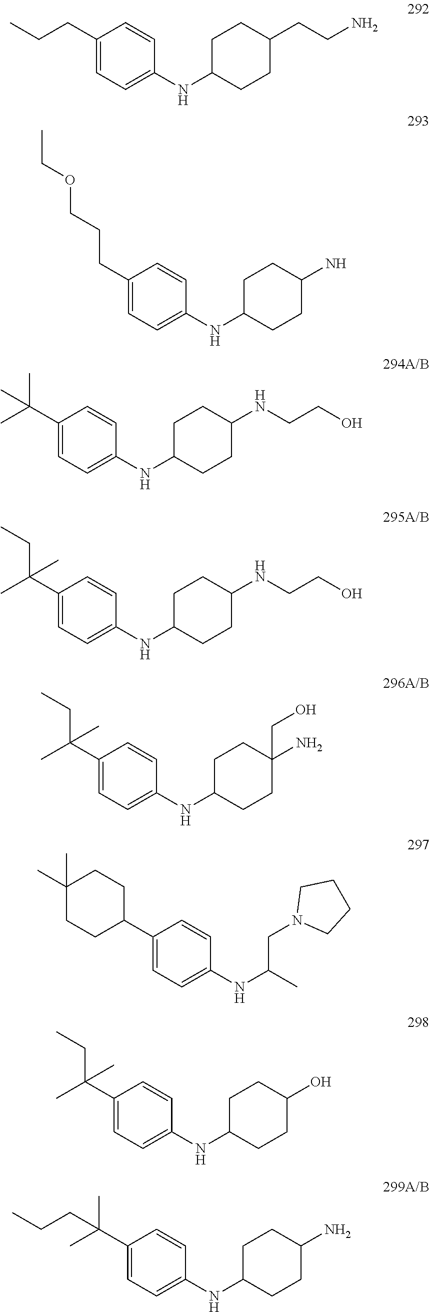

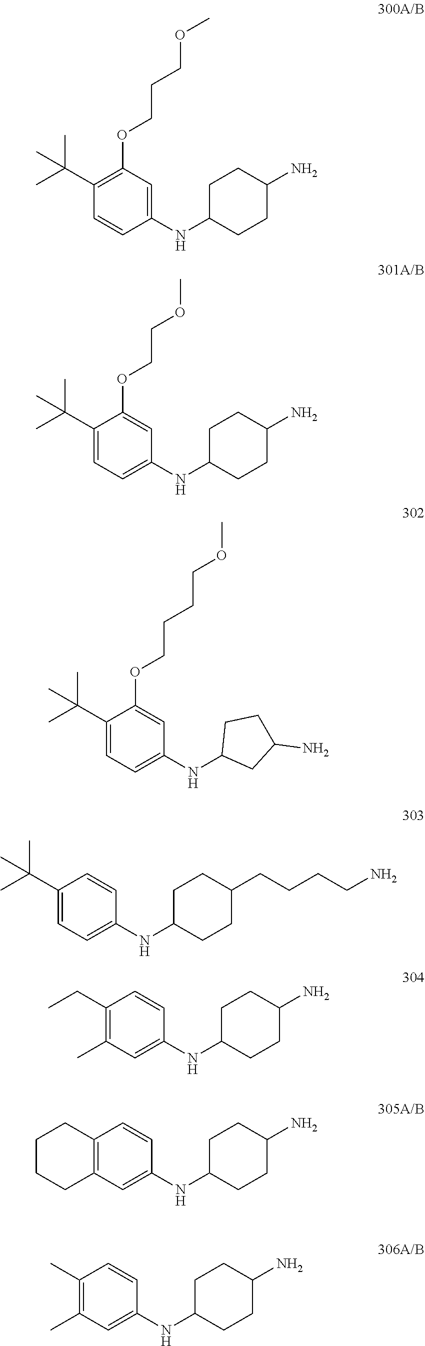

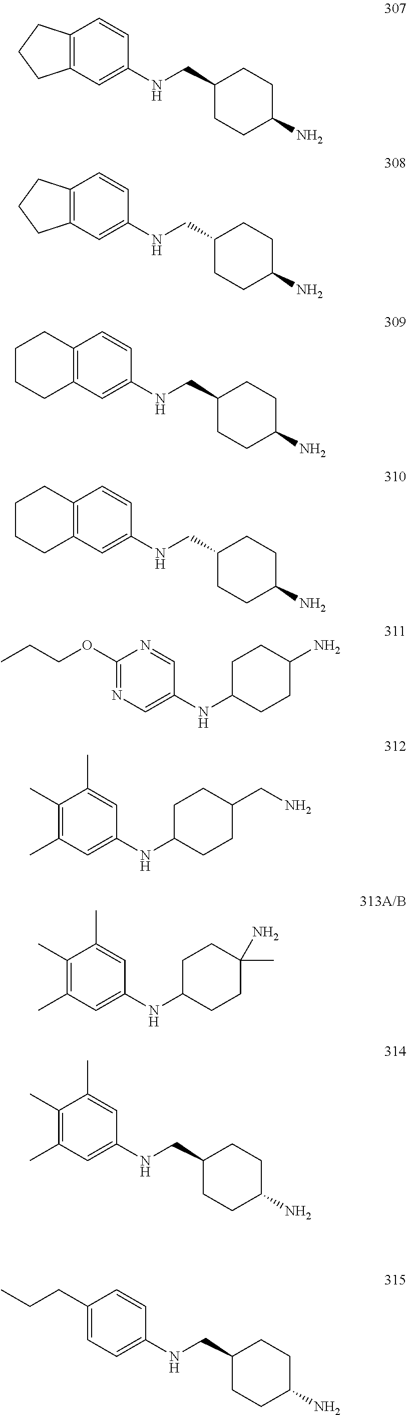

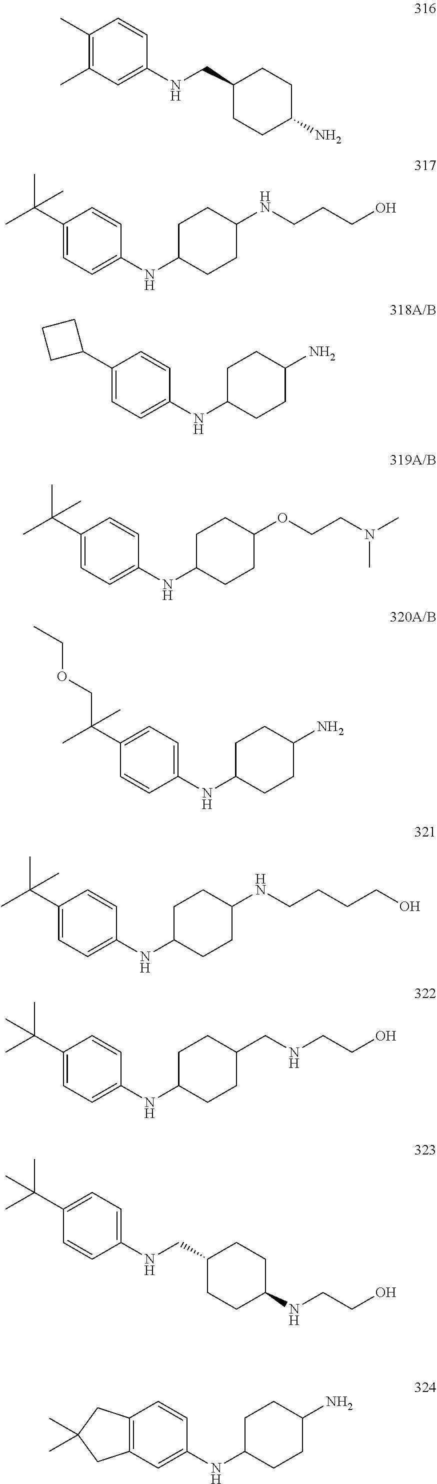

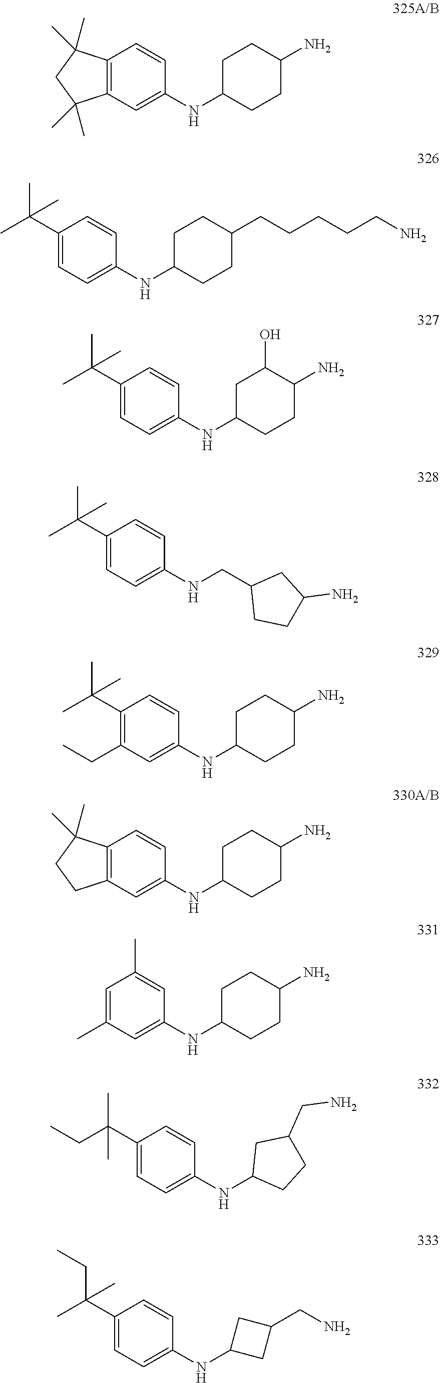

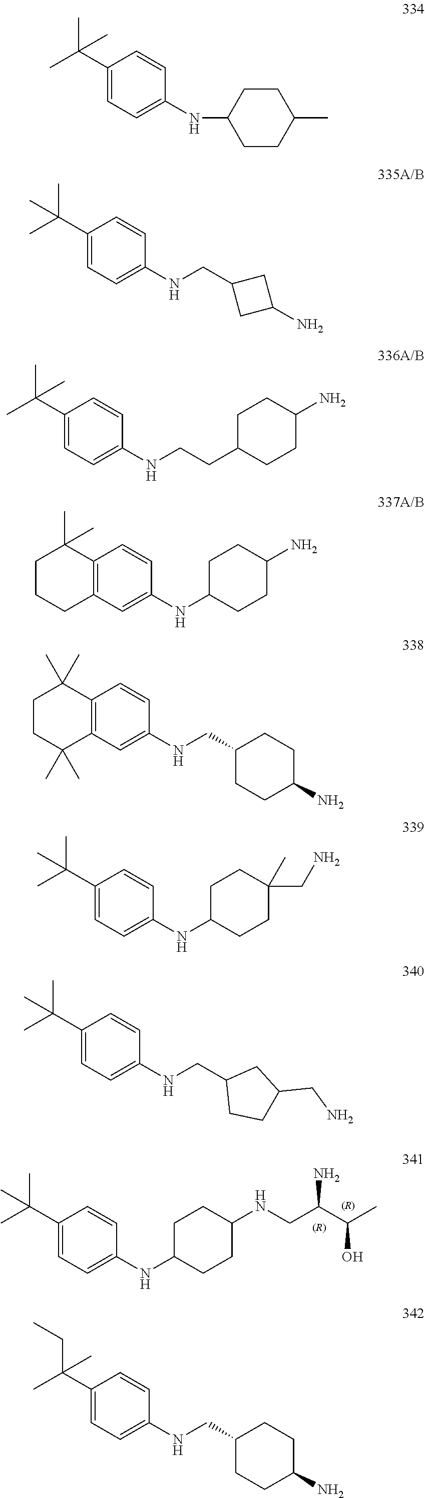

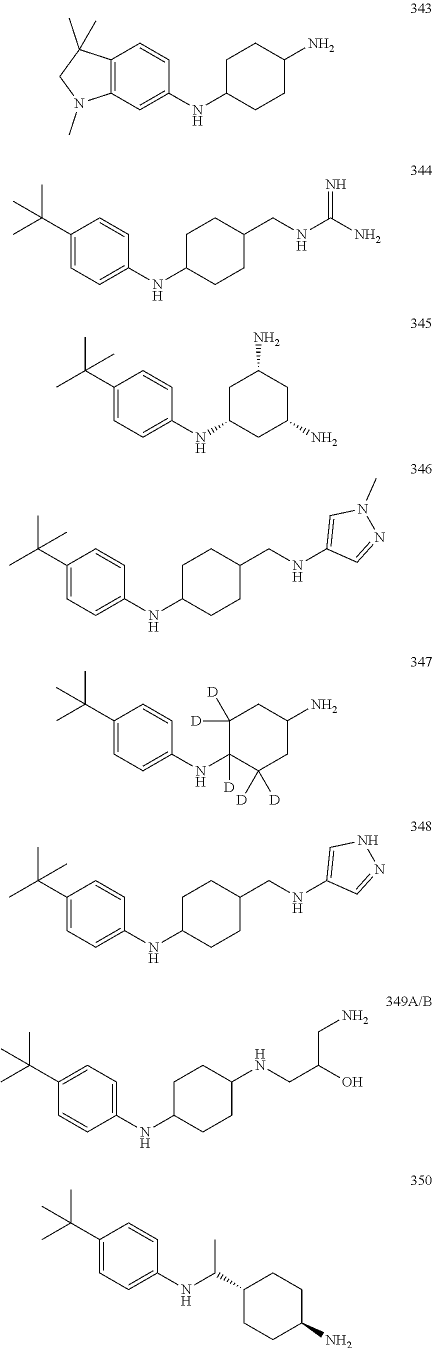

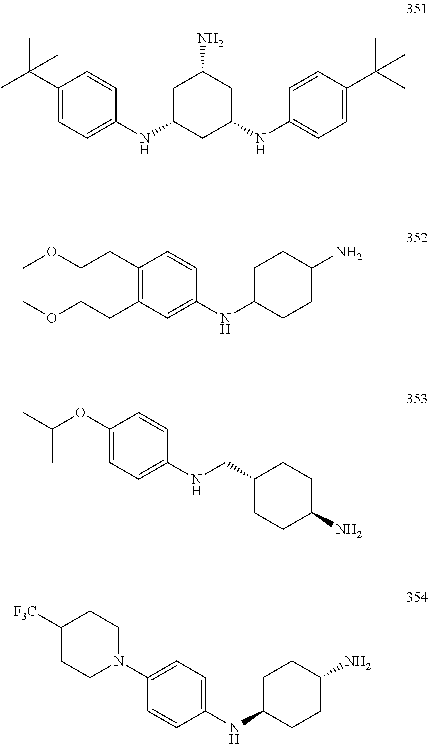

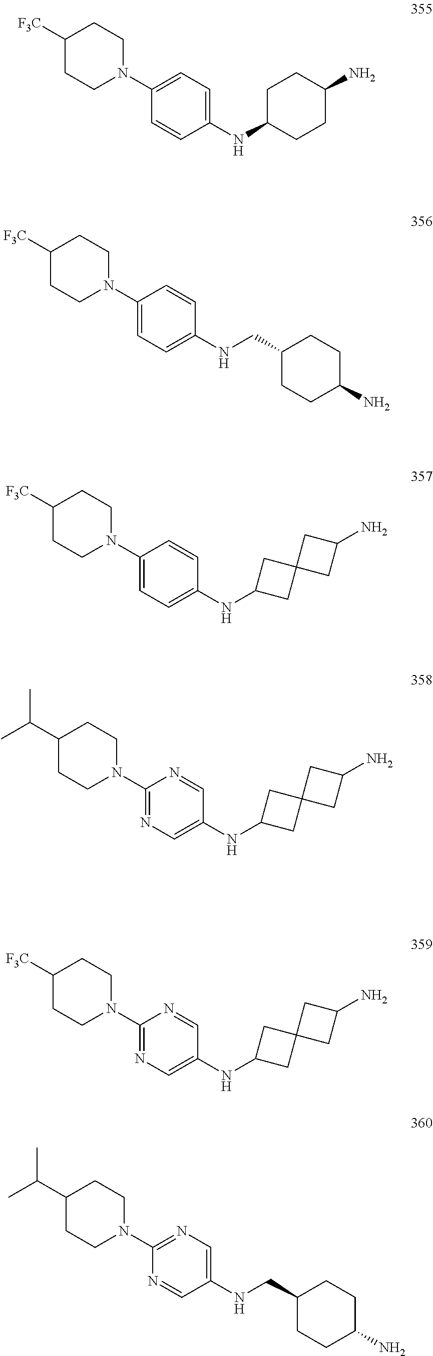 [SMILES: N[C@H]1CC[C@@H](Nc2ccc(N3CCC(C(F)(F)F)CC3)cc2)CC1 ; N[C@H]1CC[C@H](CNc2ccc(N3CCC(C(F)(F)F)CC3)cc2)CC1 ; NC1CC2(C1)CC(Nc1ccc(N3CCC(C(F)(F)F)CC3)cc1)C2 ; CC(C)C1CCN(c2ncc(NC3CC4(CC(N)C4)C3)cn2)CC1 ; NC1CC2(C1)CC(Nc1cnc(N3CCC(C(F)(F)F)CC3)nc1)C2 ; CC(C)C1CCN(c2ncc(NC[C@H]3CC[C@H](N)CC3)cn2)CC1]

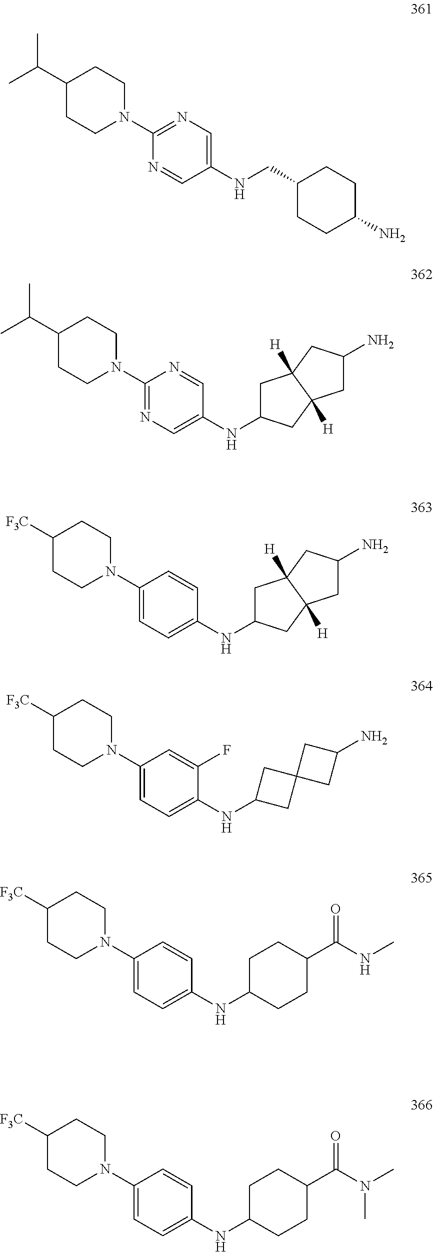 [SMILES: CC(C)C1CCN(c2ncc(NC[C@H]3CC[C@@H](N)CC3)cn2)CC1 ; CC(C)C1CCN(c2ncc(NC3C[C@H]4CC(N)C[C@H]4C3)cn2)CC1 ; NC1C[C@@H]2CC(Nc3ccc(N4CCC(C(F)(F)F)CC4)cc3)C[C@@H]2C1 ; NC1CC2(C1)CC(Nc1ccc(N3CCC(C(F)(F)F)CC3)cc1F)C2 ; CNC(=O)C1CCC(Nc2ccc(N3CCC(C(F)(F)F)CC3)cc2)CC1 ; CN(C)C(=O)C1CCC(Nc2ccc(N3CCC(C(F)(F)F)CC3)cc2)CC1]

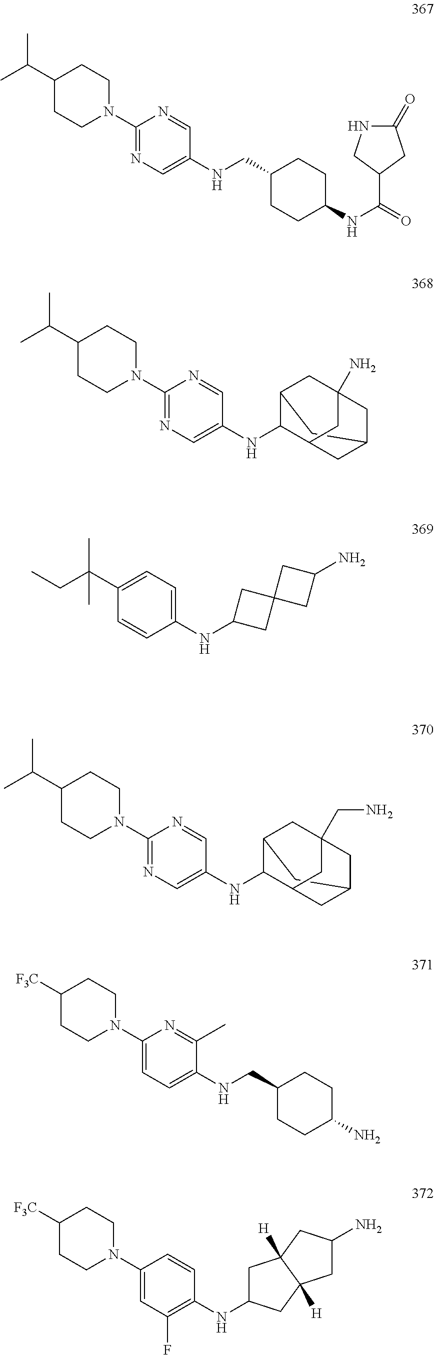 [SMILES: CC(C)C1CCN(c2ncc(NC[C@H]3CC[C@H](NC(=O)C4CNC(=O)C4)CC3)cn2)CC1 ; CC(C)C1CCN(c2ncc(NC3C4CC5CC3CC(N)(C5)C4)cn2)CC1 ; CCC(C)(C)c1ccc(NC2CC3(CC(N)C3)C2)cc1 ; CC(C)C1CCN(c2ncc(NC3C4CC5CC3CC(CN)(C5)C4)cn2)CC1 ; Cc1nc(N2CCC(C(F)(F)F)CC2)ccc1NC[C@H]1CC[C@H](N)CC1 ; NC1C[C@@H]2CC(Nc3ccc(N4CCC(C(F)(F)F)CC4)cc3F)C[C@@H]2C1]

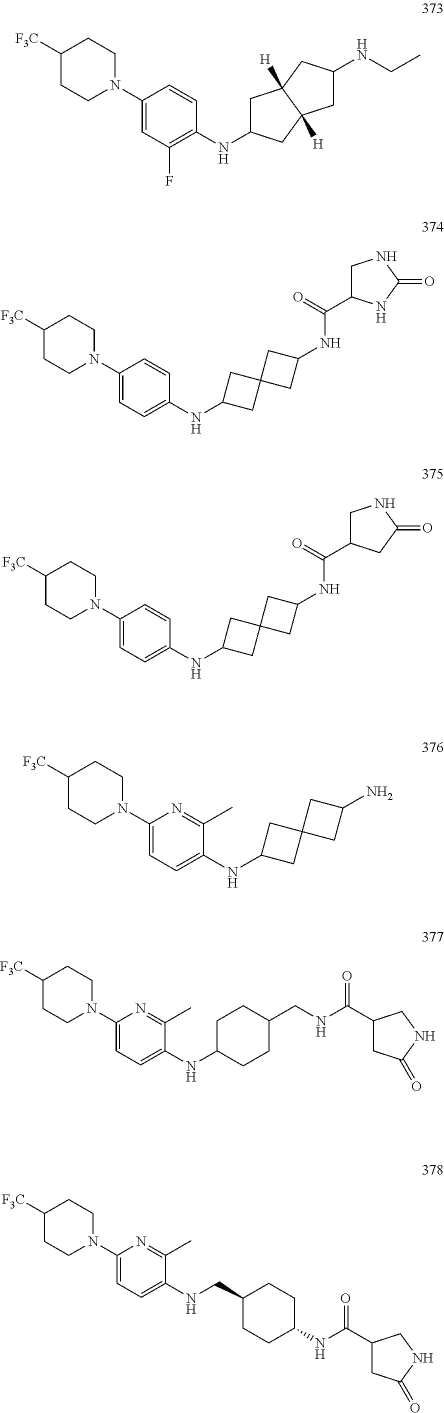 [SMILES: CCNC1C[C@@H]2CC(Nc3ccc(N4CCC(C(F)(F)F)CC4)cc3F)C[C@@H]2C1 ; O=C1NCC(C(=O)NC2CC3(C2)CC(Nc2ccc(N4CCC(C(F)(F)F)CC4)cc2)C3)N1 ; O=C1CC(C(=O)NC2CC3(C2)CC(Nc2ccc(N4CCC(C(F)(F)F)CC4)cc2)C3)CN1 ; Cc1nc(N2CCC(C(F)(F)F)CC2)ccc1NC1CC2(CC(N)C2)C1 ; Cc1nc(N2CCC(C(F)(F)F)CC2)ccc1NC1CCC(CNC(=O)C2CNC(=O)C2)CC1 ; Cc1nc(N2CCC(C(F)(F)F)CC2)ccc1NC[C@H]1CC[C@H](NC(=O)C2CNC(=O)C2)CC1]

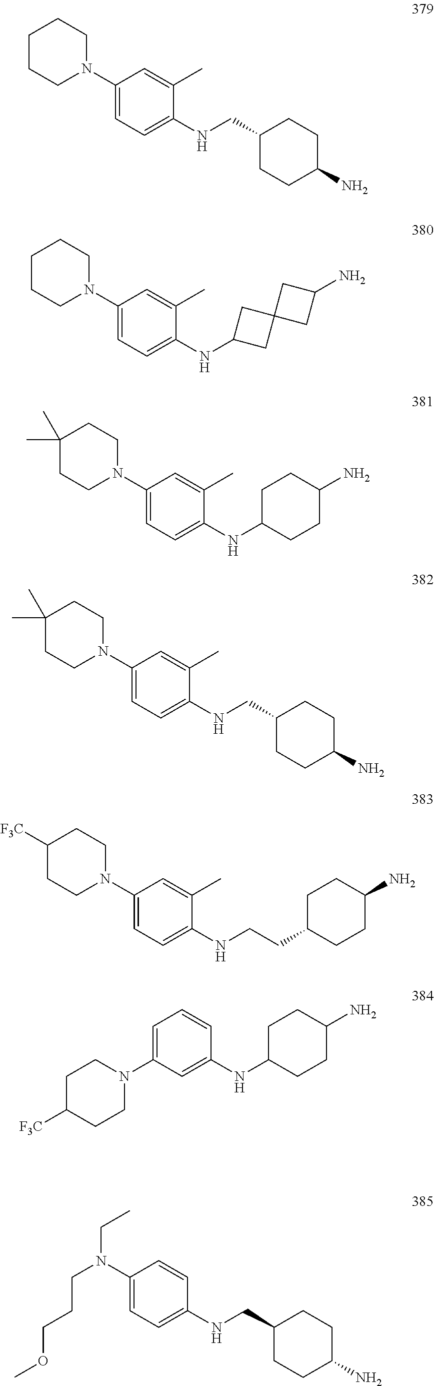

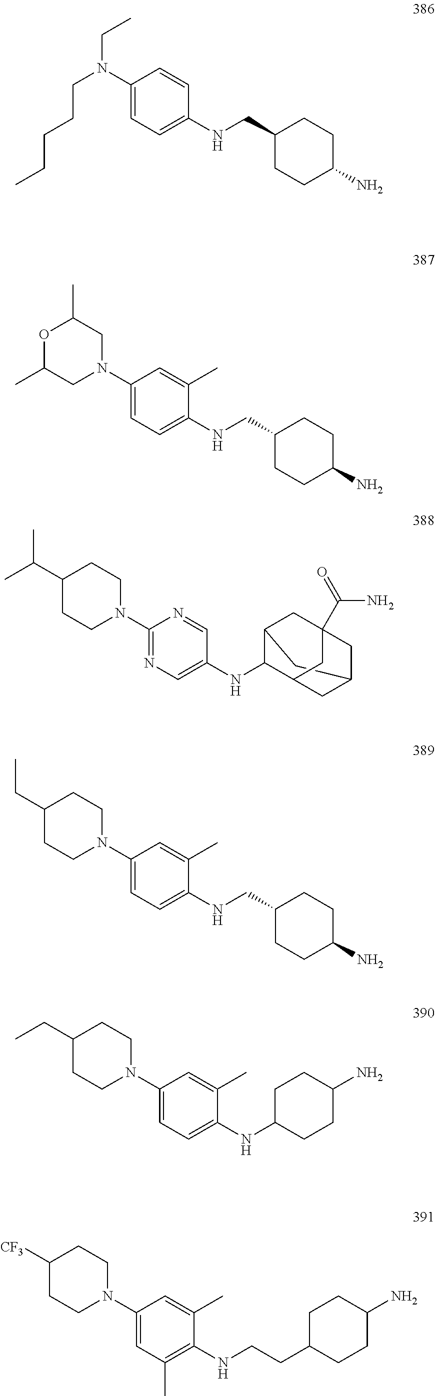 [SMILES: CCCCCN(CC)c1ccc(NC[C@H]2CC[C@H](N)CC2)cc1 ; Cc1cc(N2CC(C)OC(C)C2)ccc1NC[C@H]1CC[C@H](N)CC1 ; CC(C)C1CCN(c2ncc(NC3C4CC5CC3CC(C(N)=O)(C5)C4)cn2)CC1 ; CCC1CCN(c2ccc(NC[C@H]3CC[C@H](N)CC3)c(C)c2)CC1 ; CCC1CCN(c2ccc(NC3CCC(N)CC3)c(C)c2)CC1 ; Cc1cc(N2CCC(C(F)(F)F)CC2)cc(C)c1NCCC1CCC(N)CC1]

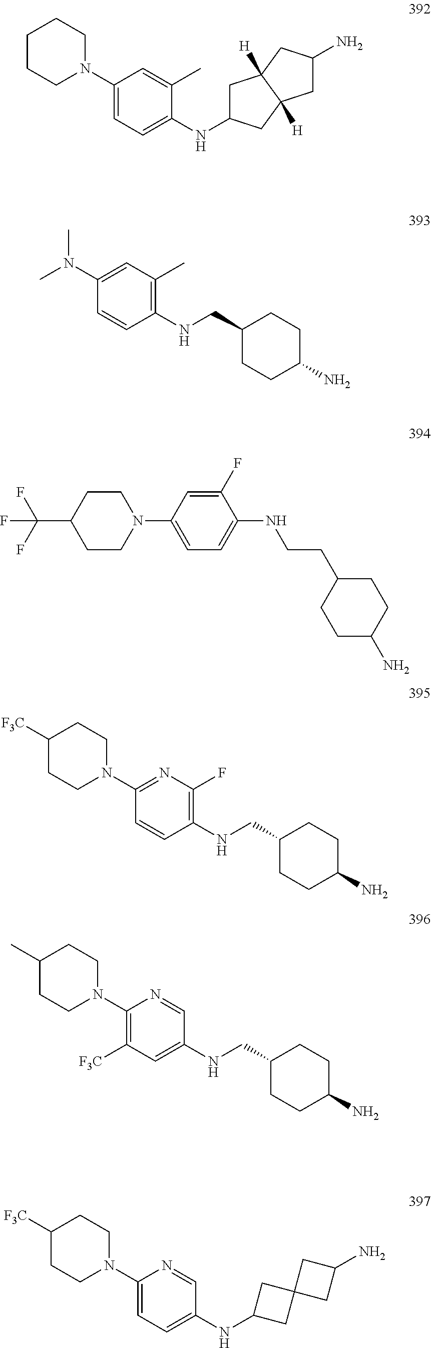 [SMILES: Cc1cc(N2CCCCC2)ccc1NC1C[C@H]2CC(N)C[C@H]2C1 ; Cc1cc(N(C)C)ccc1NC[C@H]1CC[C@H](N)CC1 ; NC1CCC(CCNc2ccc(N3CCC(C(F)(F)F)CC3)cc2F)CC1 ; N[C@H]1CC[C@H](CNc2ccc(N3CCC(C(F)(F)F)CC3)nc2F)CC1 ; CC1CCN(c2ncc(NC[C@H]3CC[C@H](N)CC3)cc2C(F)(F)F)CC1 ; NC1CC2(C1)CC(Nc1ccc(N3CCC(C(F)(F)F)CC3)nc1)C2]

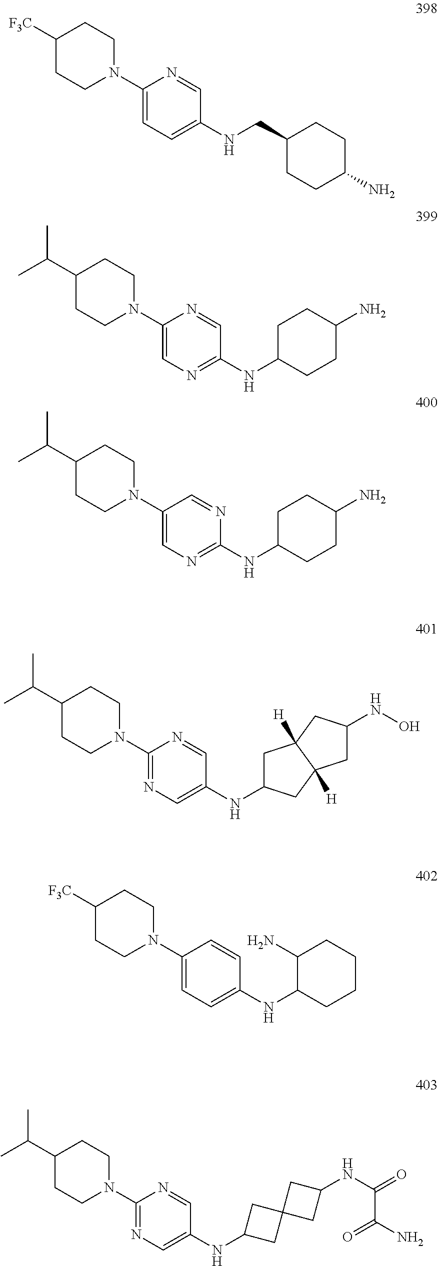 [SMILES: N[C@H]1CC[C@H](CNc2ccc(N3CCC(C(F)(F)F)CC3)nc2)CC1 ; CC(C)C1CCN(c2cnc(NC3CCC(N)CC3)cn2)CC1 ; CC(C)C1CCN(c2cnc(NC3CCC(N)CC3)nc2)CC1 ; CC(C)C1CCN(c2ncc(NC3C[C@H]4CC(NO)C[C@H]4C3)cn2)CC1 ; NC1CCCCC1Nc1ccc(N2CCC(C(F)(F)F)CC2)cc1 ; CC(C)C1CCN(c2ncc(NC3CC4(CC(NC(=O)C(N)=O)C4)C3)cn2)CC1]

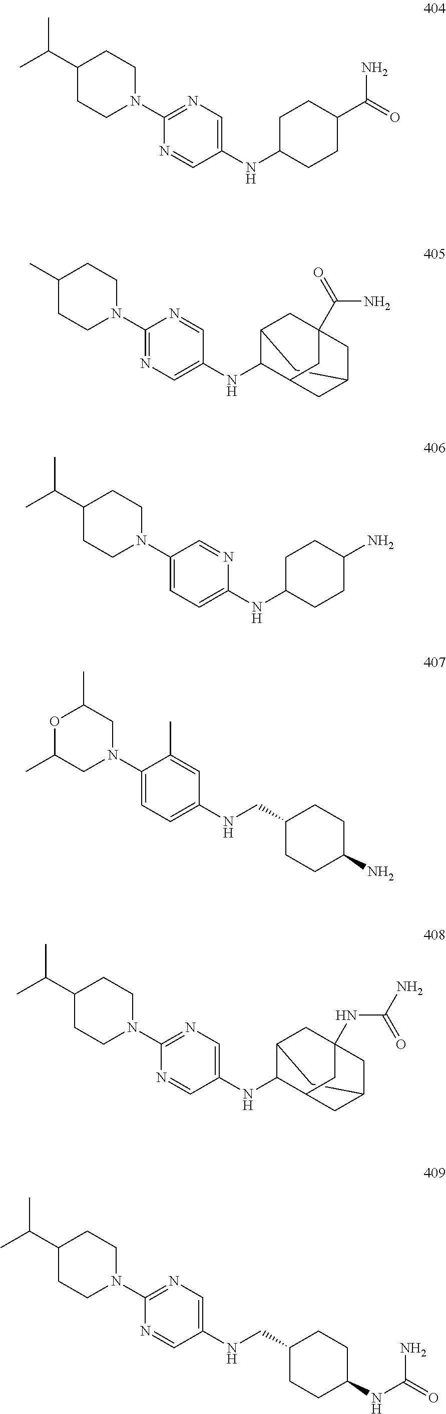 [SMILES: CC(C)C1CCN(c2ncc(NC3CCC(C(N)=O)CC3)cn2)CC1 ; CC1CCN(c2ncc(NC3C4CC5CC3CC(C(N)=O)(C5)C4)cn2)CC1 ; CC(C)C1CCN(c2ccc(NC3CCC(N)CC3)nc2)CC1 ; Cc1cc(NC[C@H]2CC[C@H](N)CC2)ccc1N1CC(C)OC(C)C1 ; CC(C)C1CCN(c2ncc(NC3C4CC5CC3CC(NC(N)=O)(C5)C4)cn2)CC1 ; CC(C)C1CCN(c2ncc(NC[C@H]3CC[C@H](NC(N)=O)CC3)cn2)CC1]

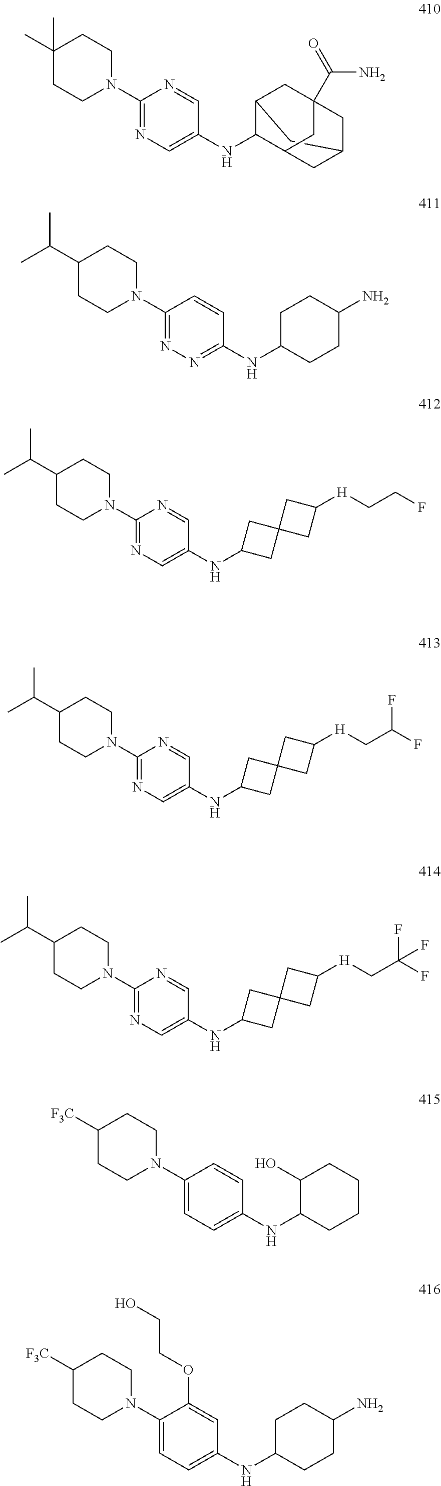

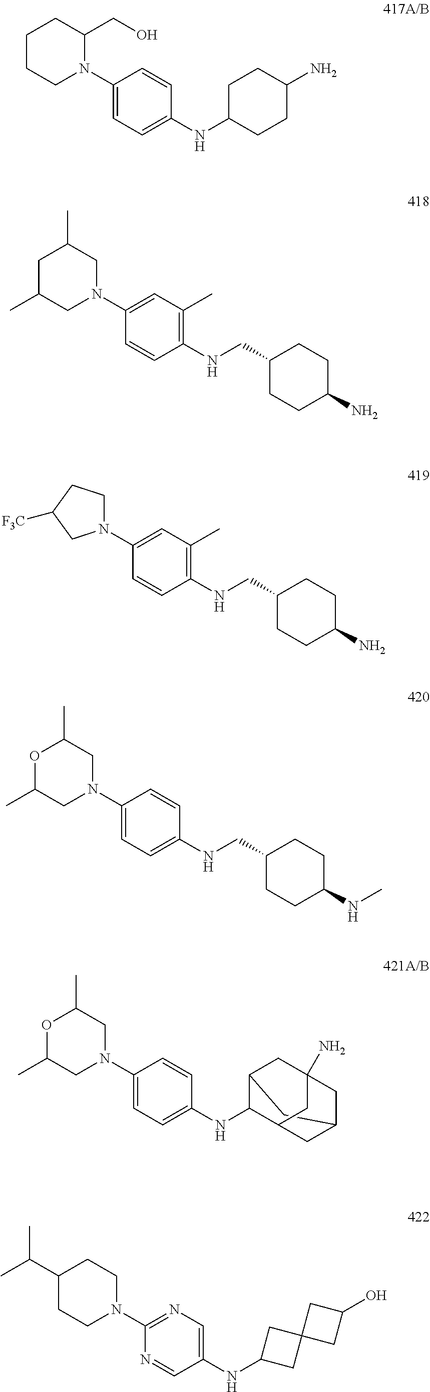

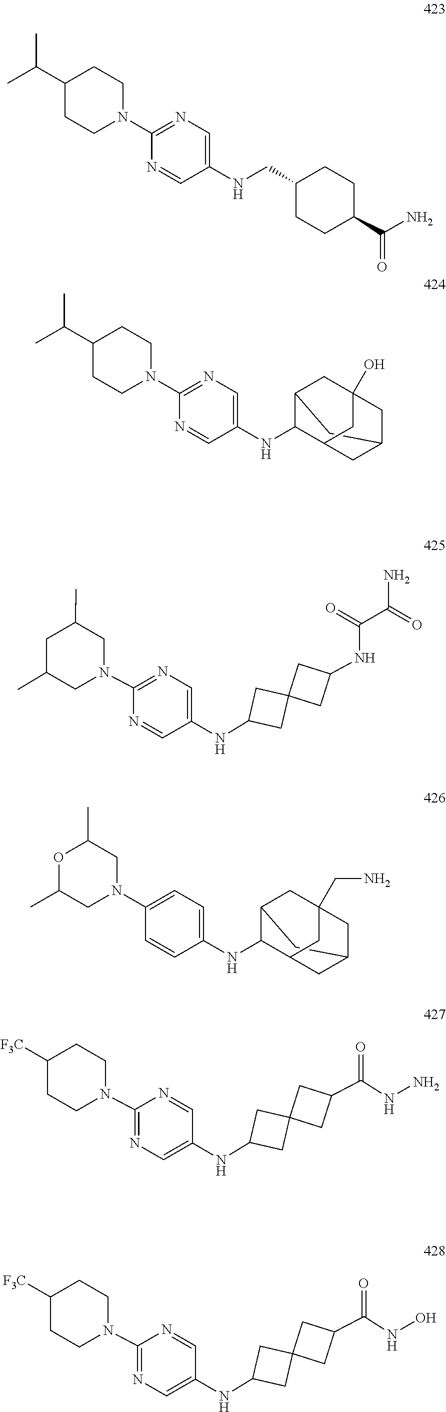 [SMILES: CC(C)C1CCN(c2ncc(NC[C@H]3CC[C@H](C(N)=O)CC3)cn2)CC1 ; CC(C)C1CCN(c2ncc(NC3C4CC5CC3CC(O)(C5)C4)cn2)CC1 ; CC1CC(C)CN(c2ncc(NC3CC4(CC(NC(=O)C(N)=O)C4)C3)cn2)C1 ; CC1CN(c2ccc(NC3C4CC5CC3CC(CN)(C5)C4)cc2)CC(C)O1 ; NNC(=O)C1CC2(CC(Nc3cnc(N4CCC(C(F)(F)F)CC4)nc3)C2)C1 ; O=C(NO)C1CC2(CC(Nc3cnc(N4CCC(C(F)(F)F)CC4)nc3)C2)C1]

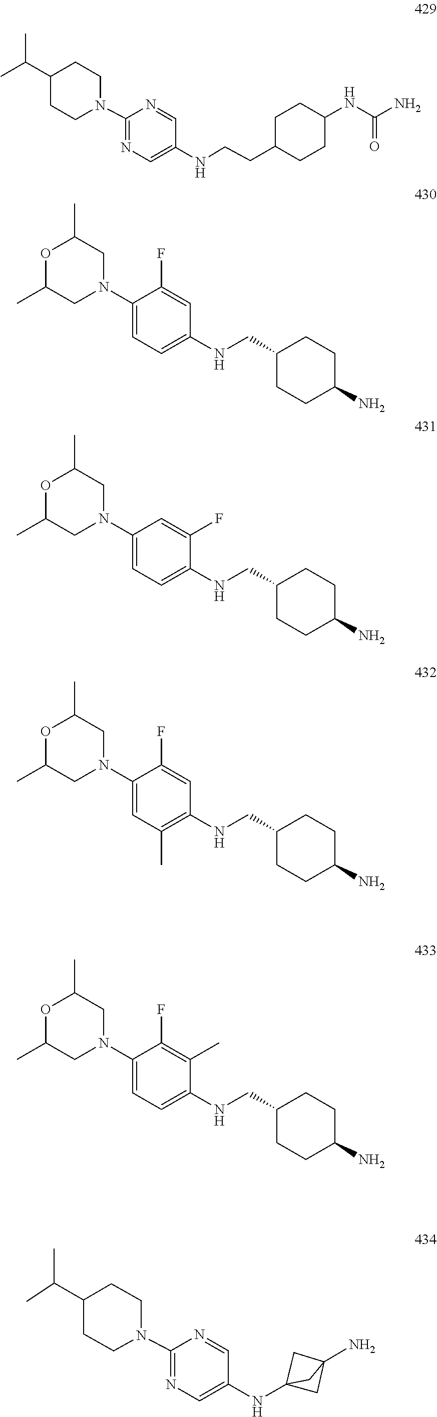 [SMILES: CC(C)C1CCN(c2ncc(NCCC3CCC(NC(N)=O)CC3)cn2)CC1 ; CC1CN(c2ccc(NC[C@H]3CC[C@H](N)CC3)cc2F)CC(C)O1 ; CC1CN(c2ccc(NC[C@H]3CC[C@H](N)CC3)c(F)c2)CC(C)O1 ; Cc1cc(N2CC(C)OC(C)C2)c(F)cc1NC[C@H]1CC[C@H](N)CC1 ; Cc1c(NC[C@H]2CC[C@H](N)CC2)ccc(N2CC(C)OC(C)C2)c1F ; CC(C)C1CCN(c2ncc(NC34CC(N)(C3)C4)cn2)CC1]

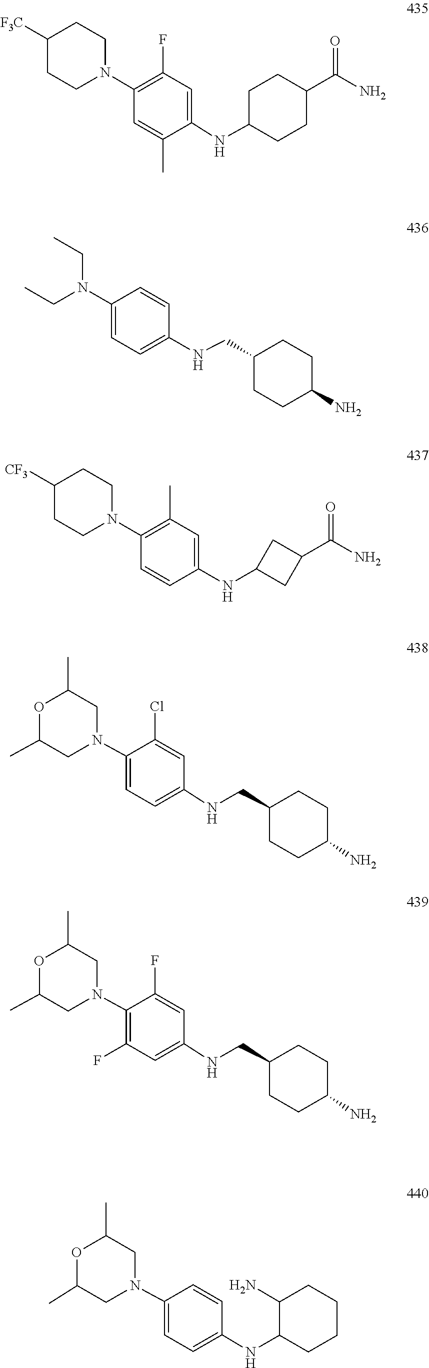 [SMILES: Cc1cc(N2CCC(C(F)(F)F)CC2)c(F)cc1NC1CCC(C(N)=O)CC1 ; CCN(CC)c1ccc(NC[C@H]2CC[C@H](N)CC2)cc1 ; Cc1cc(NC2CC(C(N)=O)C2)ccc1N1CCC(C(F)(F)F)CC1 ; CC1CN(c2ccc(NC[C@H]3CC[C@H](N)CC3)cc2Cl)CC(C)O1 ; CC1CN(c2c(F)cc(NC[C@H]3CC[C@H](N)CC3)cc2F)CC(C)O1 ; CC1CN(c2ccc(NC3CCCCC3N)cc2)CC(C)O1]

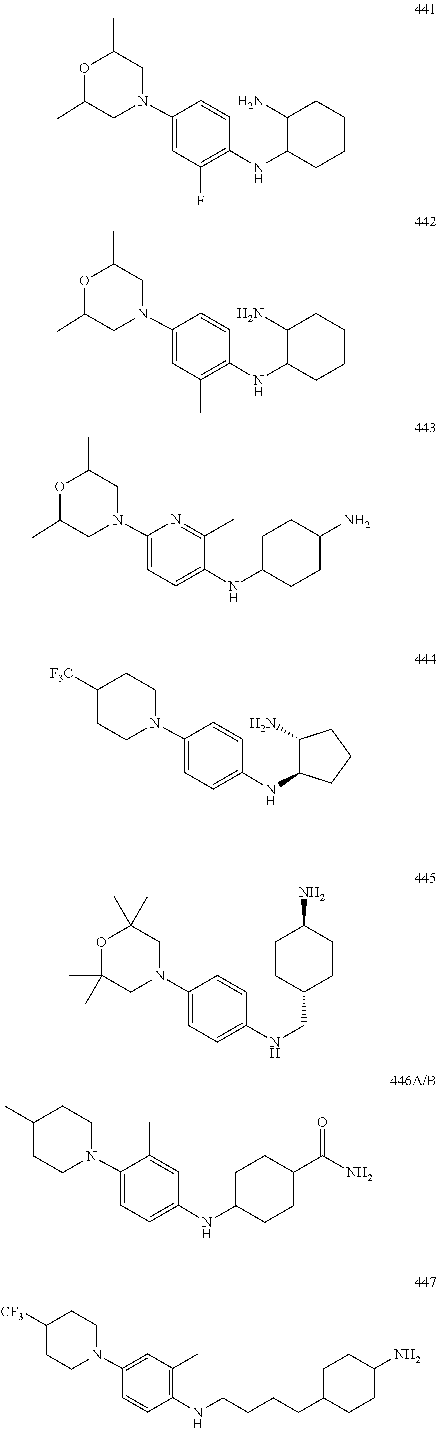

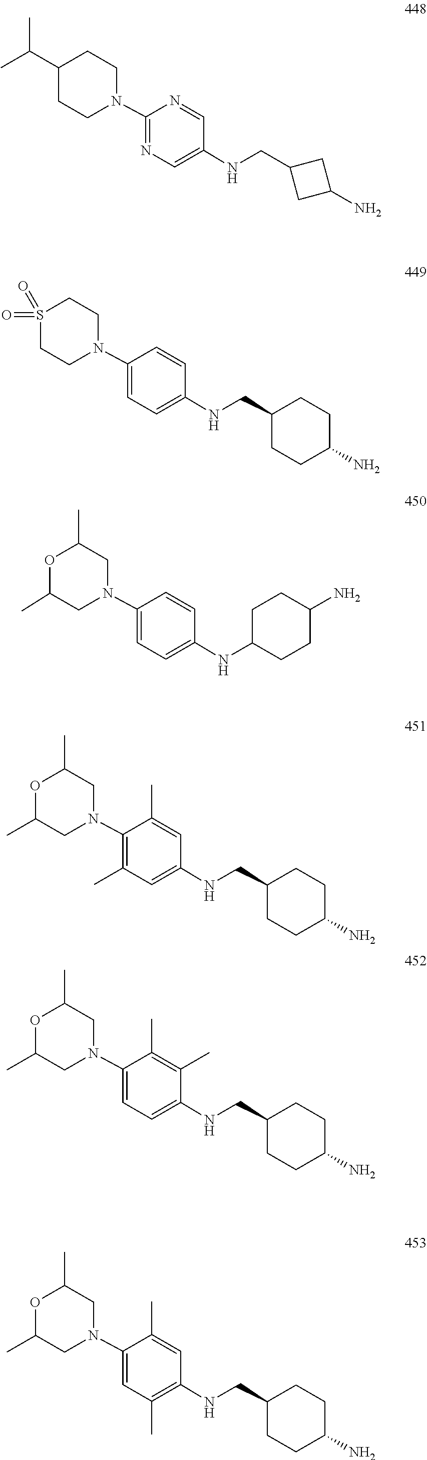 [SMILES: CC(C)C1CCN(c2ncc(NCC3CC(N)C3)cn2)CC1 ; N[C@H]1CC[C@H](CNc2ccc(N3CCS(=O)(=O)CC3)cc2)CC1 ; CC1CN(c2ccc(NC3CCC(N)CC3)cc2)CC(C)O1 ; Cc1cc(NC[C@H]2CC[C@H](N)CC2)cc(C)c1N1CC(C)OC(C)C1 ; Cc1c(NC[C@H]2CC[C@H](N)CC2)ccc(N2CC(C)OC(C)C2)c1C ; Cc1cc(N2CC(C)OC(C)C2)c(C)cc1NC[C@H]1CC[C@H](N)CC1]

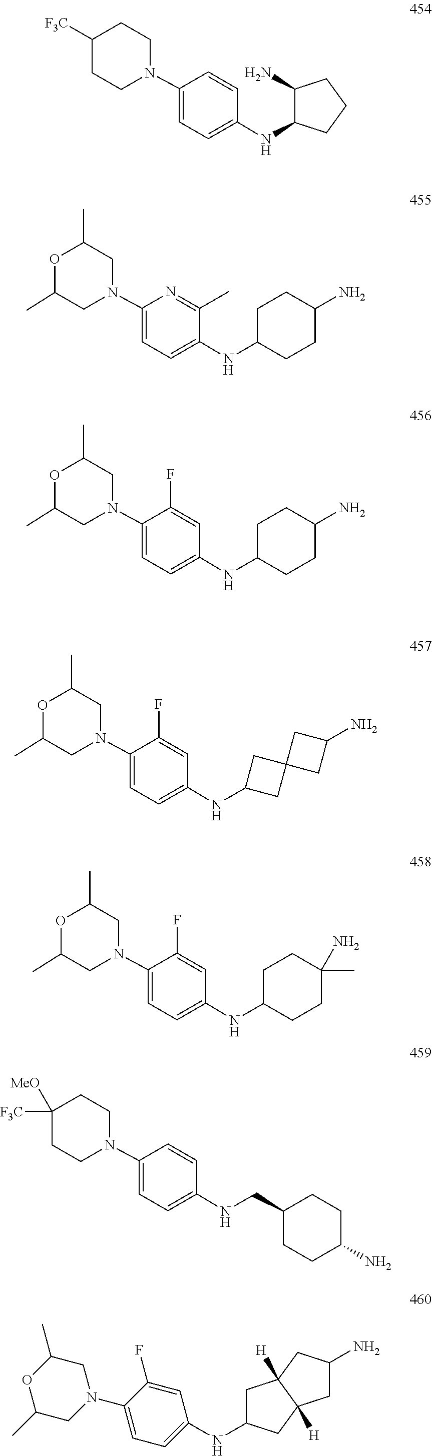

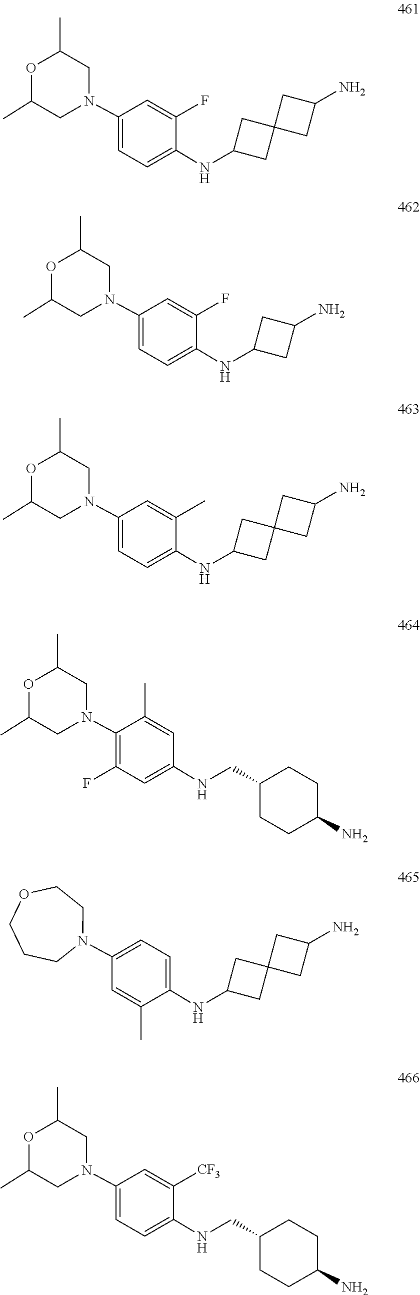 [SMILES: CC1CN(c2ccc(NC3CC4(CC(N)C4)C3)c(F)c2)CC(C)O1 ; CC1CN(c2ccc(NC3CC(N)C3)c(F)c2)CC(C)O1 ; Cc1cc(N2CC(C)OC(C)C2)ccc1NC1CC2(CC(N)C2)C1 ; Cc1cc(NC[C@H]2CC[C@H](N)CC2)cc(F)c1N1CC(C)OC(C)C1 ; Cc1cc(N2CCCOCC2)ccc1NC1CC2(CC(N)C2)C1 ; CC1CN(c2ccc(NC[C@H]3CC[C@H](N)CC3)c(C(F)(F)F)c2)CC(C)O1]

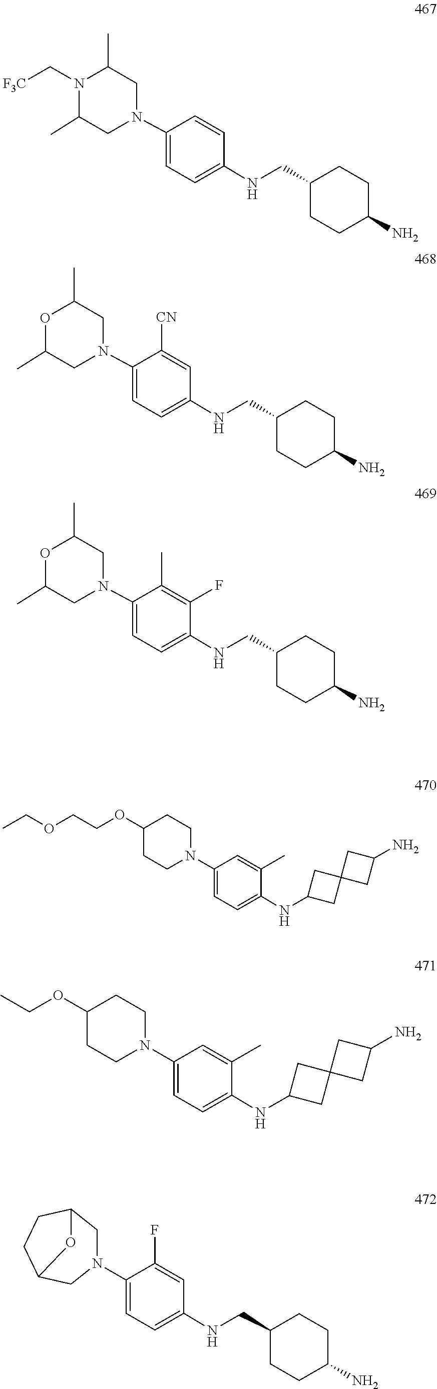 [SMILES: CC1CN(c2ccc(NC[C@H]3CC[C@H](N)CC3)cc2)CC(C)N1CC(F)(F)F ; CC1CN(c2ccc(NC[C@H]3CC[C@H](N)CC3)cc2C#N)CC(C)O1 ; Cc1c(N2CC(C)OC(C)C2)ccc(NC[C@H]2CC[C@H](N)CC2)c1F ; CCOCCOC1CCN(c2ccc(NC3CC4(CC(N)C4)C3)c(C)c2)CC1 ; CCOC1CCN(c2ccc(NC3CC4(CC(N)C4)C3)c(C)c2)CC1 ; N[C@H]1CC[C@H](CNc2ccc(N3CC4CCC(C3)O4)c(F)c2)CC1]

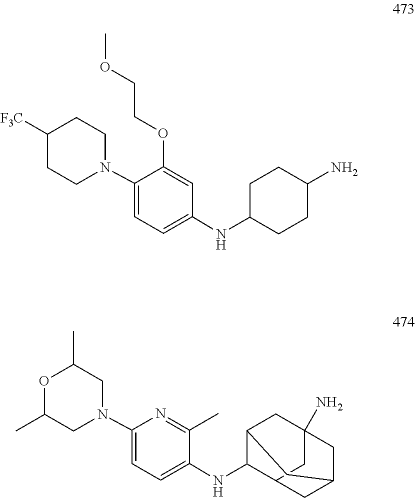 [SMILES: COCCOc1cc(NC2CCC(N)CC2)ccc1N1CCC(C(F)(F)F)CC1 ; Cc1nc(N2CC(C)OC(C)C2)ccc1NC1C2CC3CC1CC(N)(C3)C2]

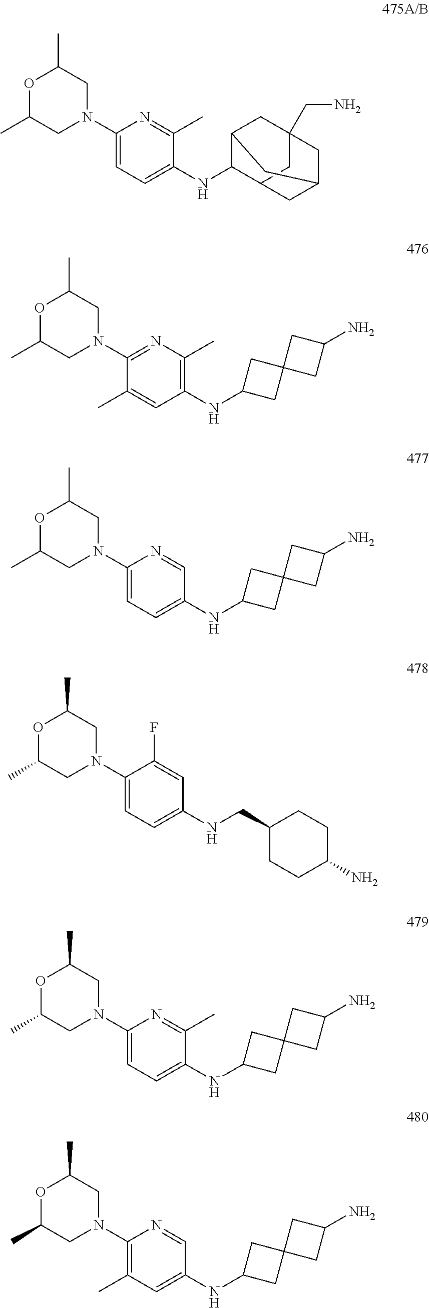 [SMILES: Cc1nc(N2CC(C)OC(C)C2)ccc1NC1C2CC3CC1CC(CN)(C3)C2 ; Cc1cc(NC2CC3(CC(N)C3)C2)c(C)nc1N1CC(C)OC(C)C1 ; CC1CN(c2ccc(NC3CC4(CC(N)C4)C3)cn2)CC(C)O1 ; C[C@H]1CN(c2ccc(NC[C@H]3CC[C@H](N)CC3)cc2F)C[C@H](C)O1 ; Cc1nc(N2C[C@H](C)O[C@@H](C)C2)ccc1NC1CC2(CC(N)C2)C1 ; Cc1cc(NC2CC3(CC(N)C3)C2)cnc1N1C[C@@H](C)O[C@@H](C)C1]

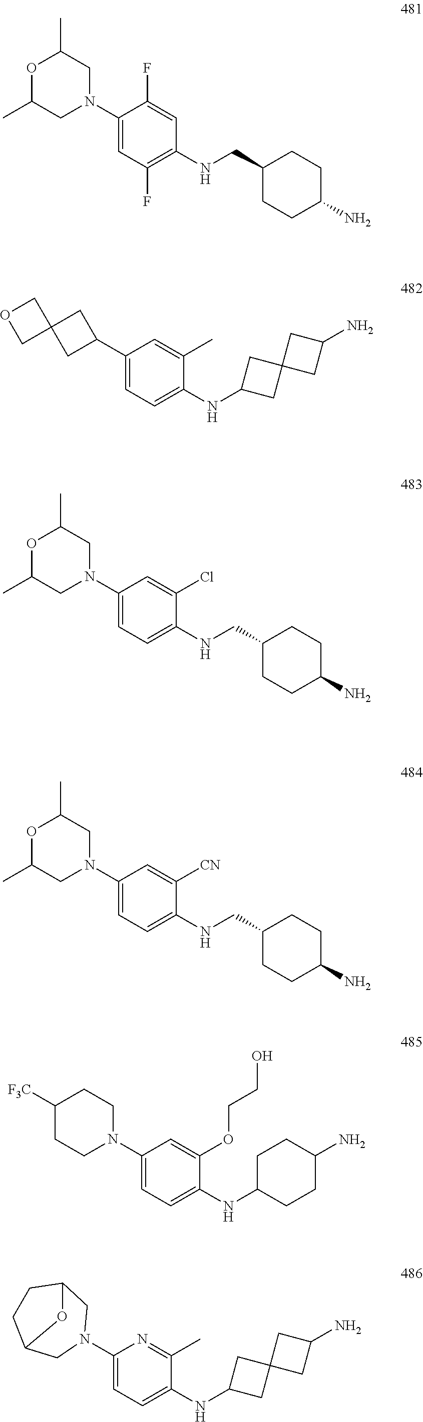 [SMILES: CC1CN(c2cc(F)c(NC[C@H]3CC[C@H](N)CC3)cc2F)CC(C)O1 ; Cc1cc(C2CC3(COC3)C2)ccc1NC1CC2(CC(N)C2)C1 ; CC1CN(c2ccc(NC[C@H]3CC[C@H](N)CC3)c(Cl)c2)CC(C)O1 ; CC1CN(c2ccc(NC[C@H]3CC[C@H](N)CC3)c(C#N)c2)CC(C)O1 ; NC1CCC(Nc2ccc(N3CCC(C(F)(F)F)CC3)cc2OCCO)CC1 ; Cc1nc(N2CC3CCC(C2)O3)ccc1NC1CC2(CC(N)C2)C1]

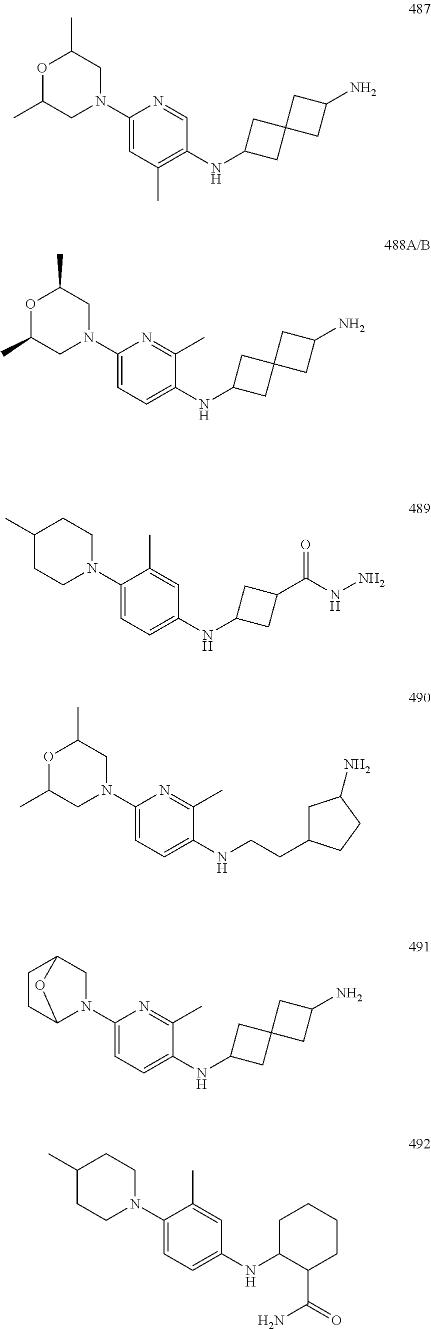 [SMILES: Cc1cc(N2CC(C)OC(C)C2)ncc1NC1CC2(CC(N)C2)C1 ; Cc1nc(N2C[C@@H](C)O[C@@H](C)C2)ccc1NC1CC2(CC(N)C2)C1 ; Cc1cc(NC2CC(C(=O)NN)C2)ccc1N1CCC(C)CC1 ; Cc1nc(N2CC(C)OC(C)C2)ccc1NCCC1CCC(N)C1 ; Cc1nc(N2CC3CCC2O3)ccc1NC1CC2(CC(N)C2)C1 ; Cc1cc(NC2CCCCC2C(N)=O)ccc1N1CCC(C)CC1]

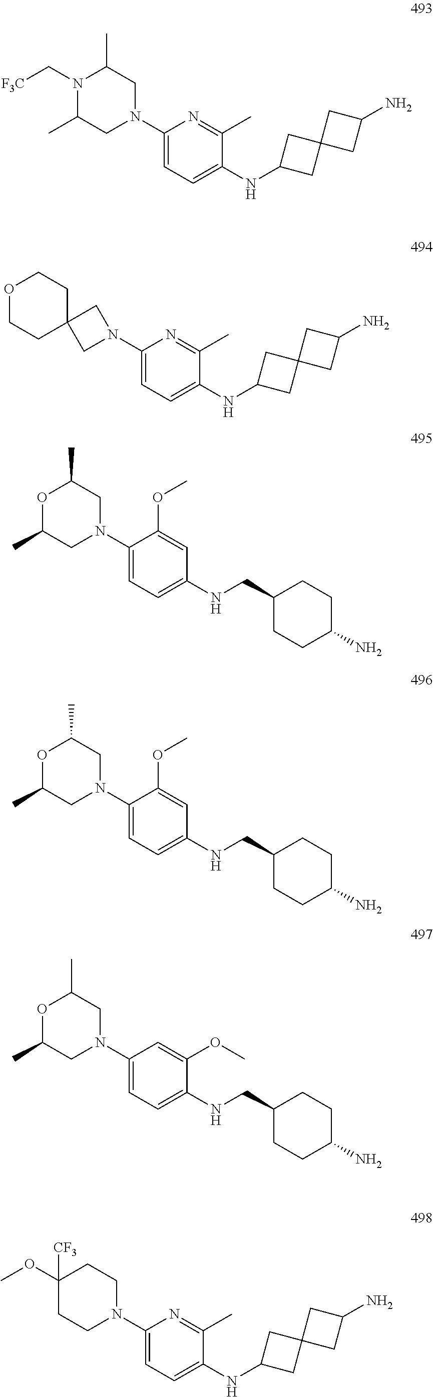 [SMILES: Cc1nc(N2CC(C)N(CC(F)(F)F)C(C)C2)ccc1NC1CC2(CC(N)C2)C1 ; Cc1nc(N2CC3(CCOCC3)C2)ccc1NC1CC2(CC(N)C2)C1 ; COc1cc(NC[C@H]2CC[C@H](N)CC2)ccc1N1C[C@@H](C)O[C@@H](C)C1 ; COc1cc(NC[C@H]2CC[C@H](N)CC2)ccc1N1C[C@@H](C)O[C@H](C)C1 ; COc1cc(N2CC(C)O[C@H](C)C2)ccc1NC[C@H]1CC[C@H](N)CC1 ; COC1(C(F)(F)F)CCN(c2ccc(NC3CC4(CC(N)C4)C3)c(C)n2)CC1]

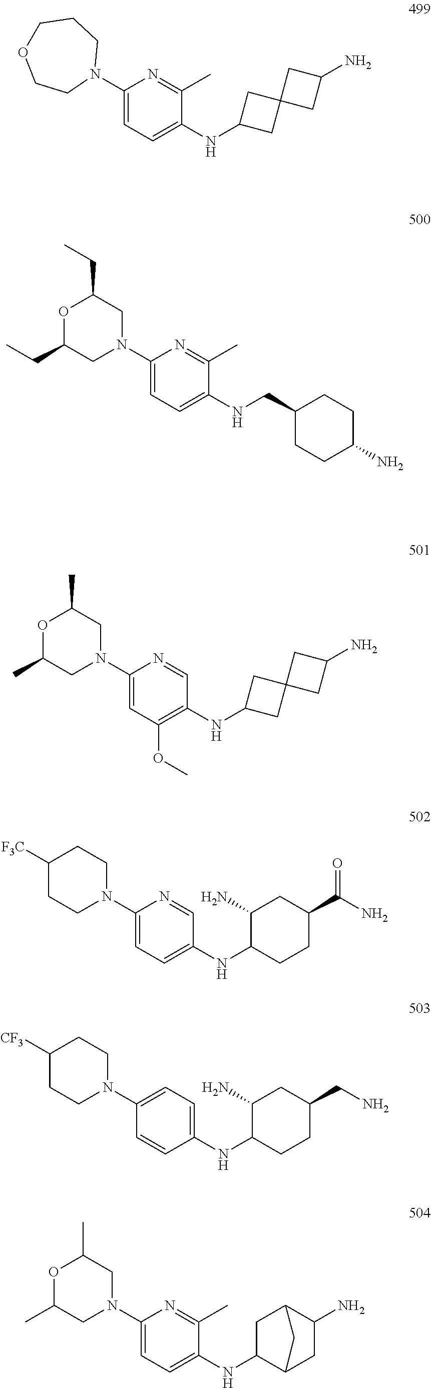 [SMILES: Cc1nc(N2CCCOCC2)ccc1NC1CC2(CC(N)C2)C1 ; CC[C@@H]1CN(c2ccc(NC[C@H]3CC[C@H](N)CC3)c(C)n2)C[C@H](CC)O1 ; COc1cc(N2C[C@@H](C)O[C@@H](C)C2)ncc1NC1CC2(CC(N)C2)C1 ; NC(=O)[C@H]1CCC(Nc2ccc(N3CCC(C(F)(F)F)CC3)nc2)[C@H](N)C1 ; NC[C@H]1CCC(Nc2ccc(N3CCC(C(F)(F)F)CC3)cc2)[C@H](N)C1 ; Cc1nc(N2CC(C)OC(C)C2)ccc1NC1CC2CC1CC2N]

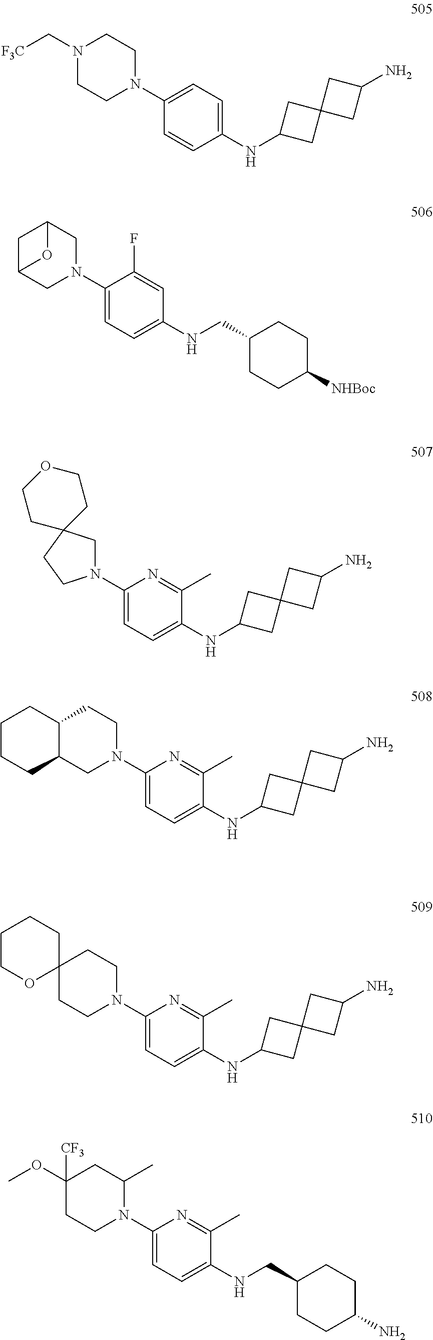 [SMILES: NC1CC2(C1)CC(Nc1ccc(N3CCN(CC(F)(F)F)CC3)cc1)C2 ; CC(C)(C)OC(=O)N[C@H]1CC[C@H](CNc2ccc(N3CC4CC(C3)O4)c(F)c2)CC1 ; Cc1nc(N2CCC3(CCOCC3)C2)ccc1NC1CC2(CC(N)C2)C1 ; Cc1nc(N2CC[C@H]3CCCC[C@@H]3C2)ccc1NC1CC2(CC(N)C2)C1 ; Cc1nc(N2CCC3(CCCCO3)CC2)ccc1NC1CC2(CC(N)C2)C1 ; COC1(C(F)(F)F)CCN(c2ccc(NC[C@H]3CC[C@H](N)CC3)c(C)n2)C(C)C1]

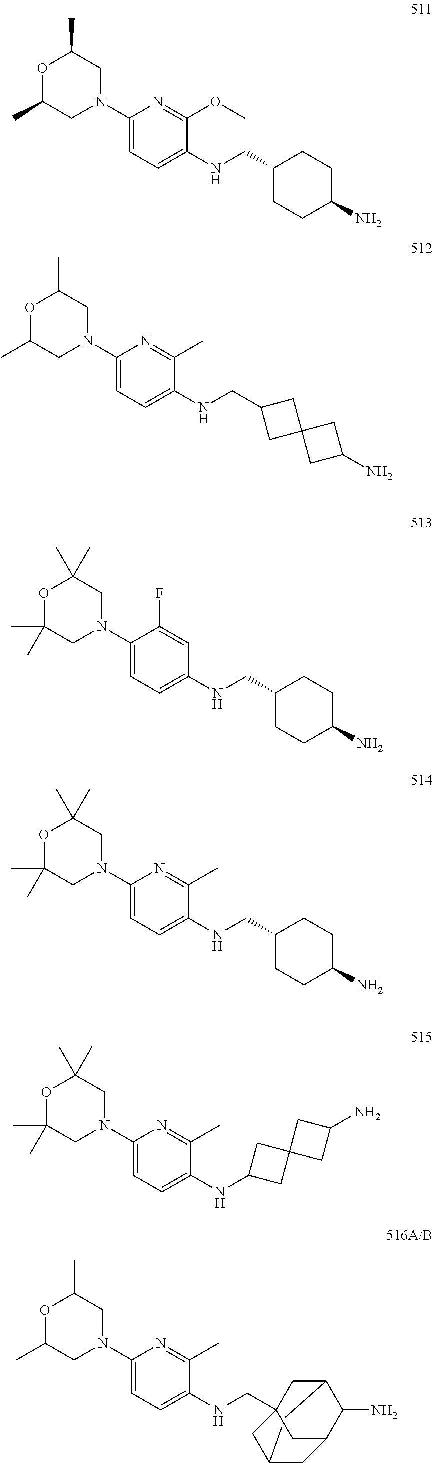 [SMILES: COc1nc(N2C[C@@H](C)O[C@@H](C)C2)ccc1NC[C@H]1CC[C@H](N)CC1 ; Cc1nc(N2CC(C)OC(C)C2)ccc1NCC1CC2(CC(N)C2)C1 ; CC1(C)CN(c2ccc(NC[C@H]3CC[C@H](N)CC3)cc2F)CC(C)(C)O1 ; Cc1nc(N2CC(C)(C)OC(C)(C)C2)ccc1NC[C@H]1CC[C@H](N)CC1 ; Cc1nc(N2CC(C)(C)OC(C)(C)C2)ccc1NC1CC2(CC(N)C2)C1 ; Cc1nc(N2CC(C)OC(C)C2)ccc1NCC12CC3CC(C1)C(N)C(C3)C2]

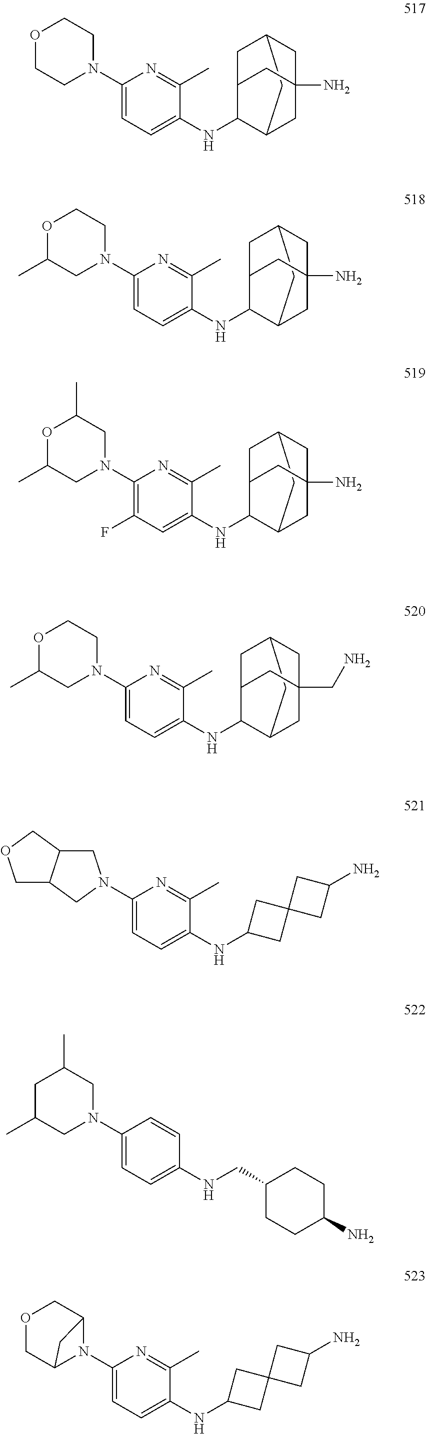

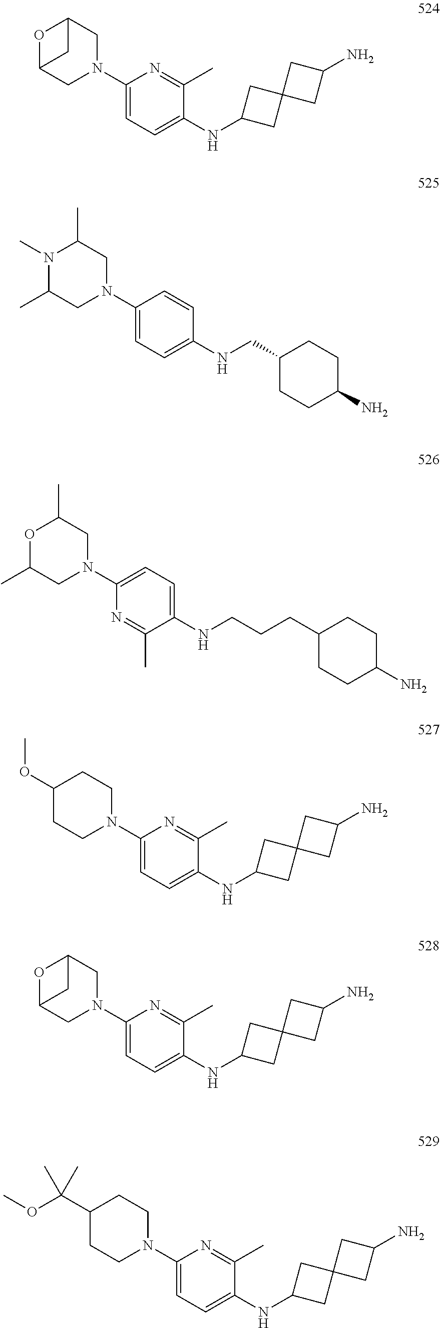 [SMILES: Cc1nc(N2CC3CC(C2)O3)ccc1NC1CC2(CC(N)C2)C1 ; CC1CN(c2ccc(NC[C@H]3CC[C@H](N)CC3)cc2)CC(C)N1C ; Cc1nc(N2CC(C)OC(C)C2)ccc1NCCCC1CCC(N)CC1 ; COC1CCN(c2ccc(NC3CC4(CC(N)C4)C3)c(C)n2)CC1 ; Cc1nc(N2CC3CC(C2)O3)ccc1NC1CC2(CC(N)C2)C1 ; COC(C)(C)C1CCN(c2ccc(NC3CC4(CC(N)C4)C3)c(C)n2)CC1]

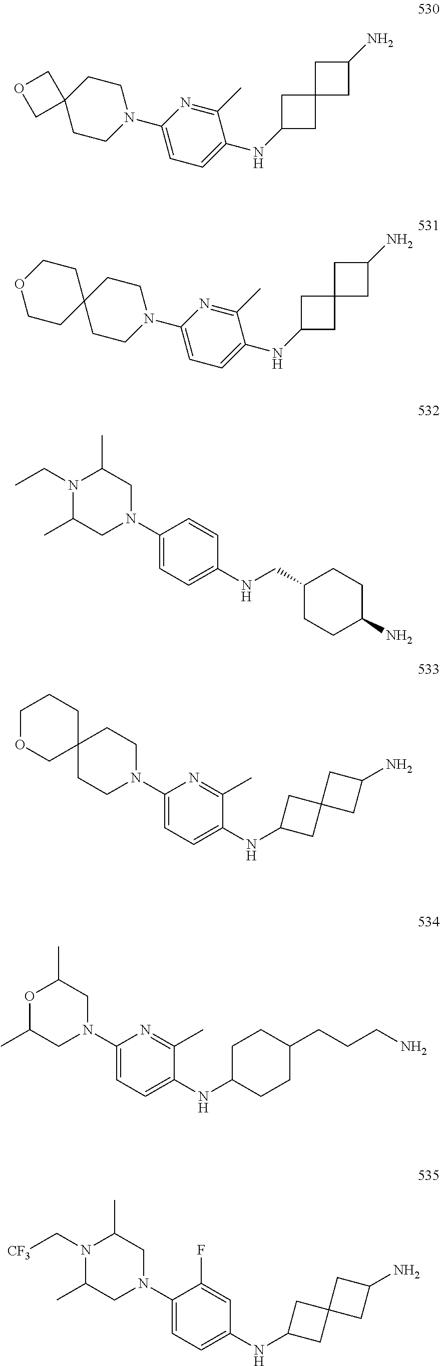 [SMILES: Cc1nc(N2CCC3(CC2)COC3)ccc1NC1CC2(CC(N)C2)C1 ; Cc1nc(N2CCC3(CCOCC3)CC2)ccc1NC1CC2(CC(N)C2)C1 ; CCN1C(C)CN(c2ccc(NC[C@H]3CC[C@H](N)CC3)cc2)CC1C ; Cc1nc(N2CCC3(CCCOC3)CC2)ccc1NC1CC2(CC(N)C2)C1 ; Cc1nc(N2CC(C)OC(C)C2)ccc1NC1CCC(CCCN)CC1 ; CC1CN(c2ccc(NC3CC4(CC(N)C4)C3)cc2F)CC(C)N1CC(F)(F)F]

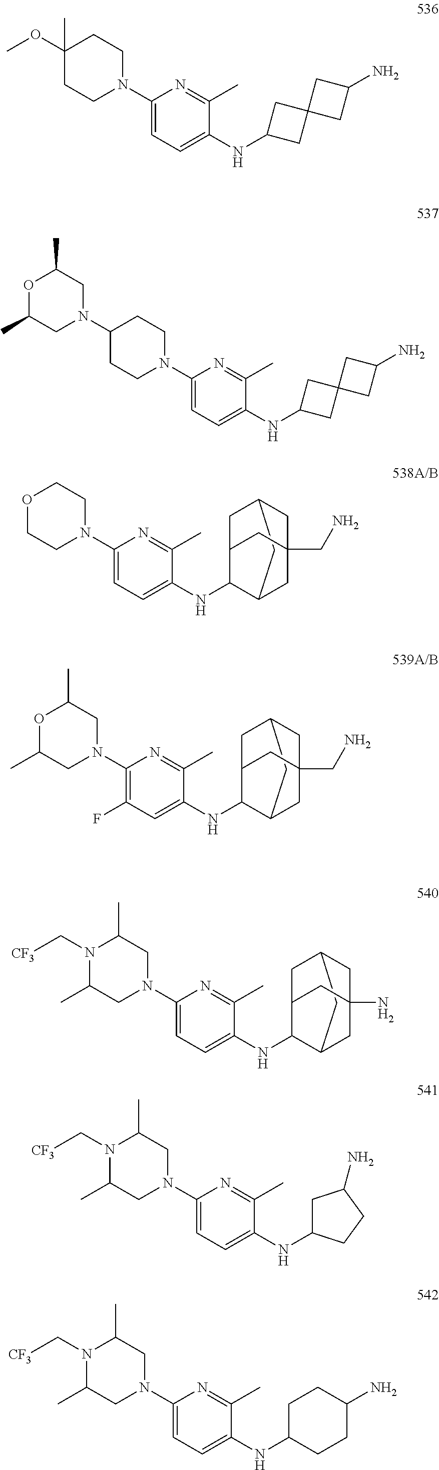

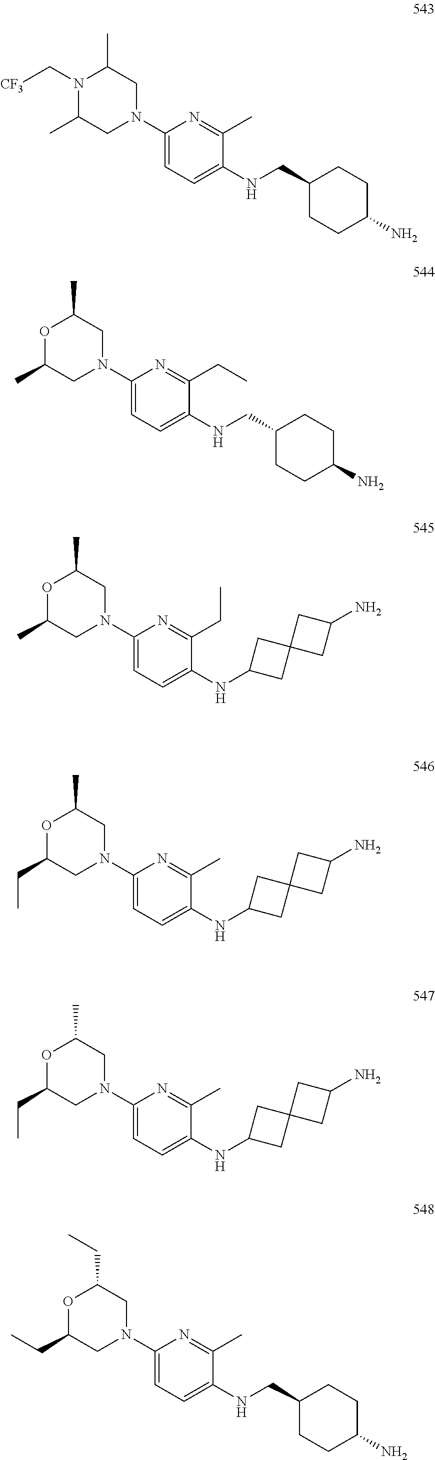 [SMILES: Cc1nc(N2CC(C)N(CC(F)(F)F)C(C)C2)ccc1NC[C@H]1CC[C@H](N)CC1 ; CCc1nc(N2C[C@@H](C)O[C@@H](C)C2)ccc1NC[C@H]1CC[C@H](N)CC1 ; CCc1nc(N2C[C@@H](C)O[C@@H](C)C2)ccc1NC1CC2(CC(N)C2)C1 ; CC[C@@H]1CN(c2ccc(NC3CC4(CC(N)C4)C3)c(C)n2)C[C@H](C)O1 ; CC[C@@H]1CN(c2ccc(NC3CC4(CC(N)C4)C3)c(C)n2)C[C@@H](C)O1 ; CC[C@@H]1CN(c2ccc(NC[C@H]3CC[C@H](N)CC3)c(C)n2)C[C@@H](CC)O1]

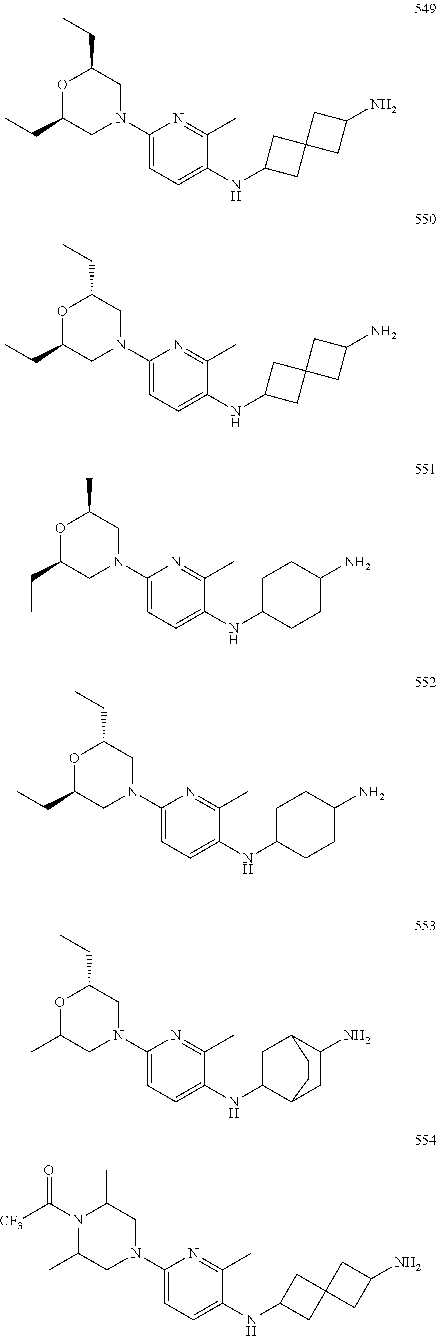 [SMILES: CC[C@@H]1CN(c2ccc(NC3CC4(CC(N)C4)C3)c(C)n2)C[C@H](CC)O1 ; CC[C@@H]1CN(c2ccc(NC3CC4(CC(N)C4)C3)c(C)n2)C[C@@H](CC)O1 ; CC[C@@H]1CN(c2ccc(NC3CCC(N)CC3)c(C)n2)C[C@H](C)O1 ; CC[C@@H]1CN(c2ccc(NC3CCC(N)CC3)c(C)n2)C[C@@H](CC)O1 ; CC[C@@H]1CN(c2ccc(NC3CC4CCC3CC4N)c(C)n2)CC(C)O1 ; Cc1nc(N2CC(C)N(C(=O)C(F)(F)F)C(C)C2)ccc1NC1CC2(CC(N)C2)C1]

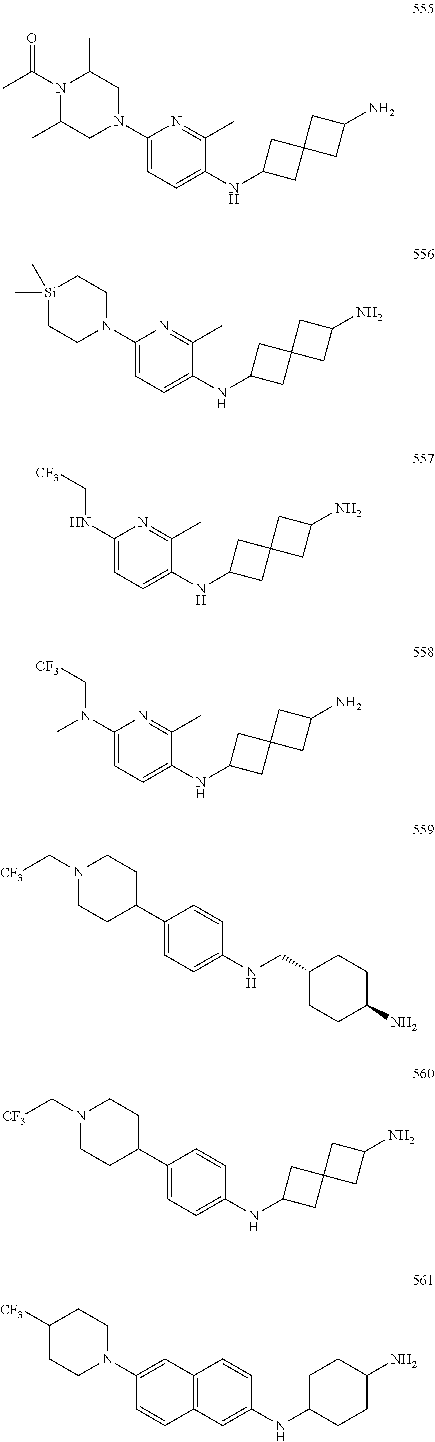

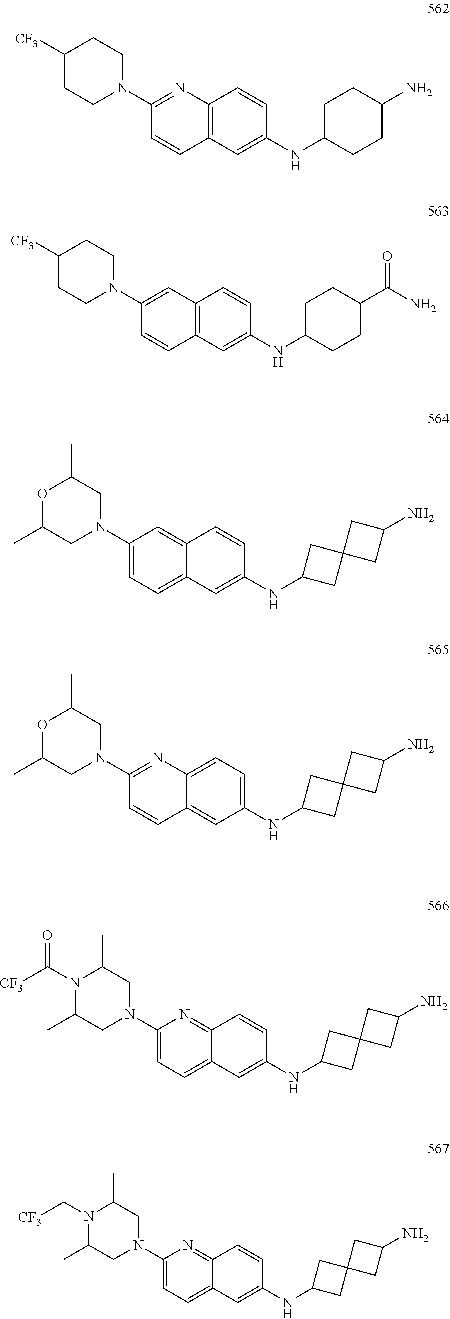 [SMILES: NC1CCC(Nc2ccc3nc(N4CCC(C(F)(F)F)CC4)ccc3c2)CC1 ; NC(=O)C1CCC(Nc2ccc3cc(N4CCC(C(F)(F)F)CC4)ccc3c2)CC1 ; CC1CN(c2ccc3cc(NC4CC5(CC(N)C5)C4)ccc3c2)CC(C)O1 ; CC1CN(c2ccc3cc(NC4CC5(CC(N)C5)C4)ccc3n2)CC(C)O1 ; CC1CN(c2ccc3cc(NC4CC5(CC(N)C5)C4)ccc3n2)CC(C)N1C(=O)C(F)(F)F ; CC1CN(c2ccc3cc(NC4CC5(CC(N)C5)C4)ccc3n2)CC(C)N1CC(F)(F)F]

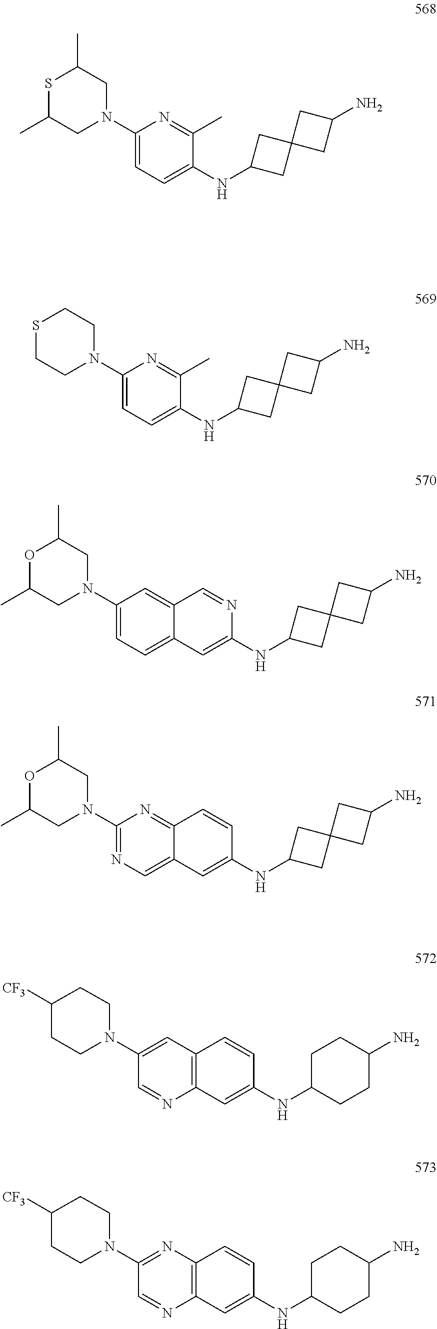 [SMILES: Cc1nc(N2CC(C)SC(C)C2)ccc1NC1CC2(CC(N)C2)C1 ; Cc1nc(N2CCSCC2)ccc1NC1CC2(CC(N)C2)C1 ; CC1CN(c2ccc3cc(NC4CC5(CC(N)C5)C4)ncc3c2)CC(C)O1 ; CC1CN(c2ncc3cc(NC4CC5(CC(N)C5)C4)ccc3n2)CC(C)O1 ; NC1CCC(Nc2ccc3cc(N4CCC(C(F)(F)F)CC4)cnc3c2)CC1 ; NC1CCC(Nc2ccc3nc(N4CCC(C(F)(F)F)CC4)cnc3c2)CC1]

a tautomer thereof, a solvate or stereoisomer of the compound or the tautomer, or a pharmaceutically acceptable salt of the foregoing.

45. A pharmaceutical composition comprising a compound according to any one of technical solutions 1 to 44, a tautomer thereof, a solvate or stereoisomer of the compound or the tautomer, or a pharmaceutically acceptable salt of the foregoing and at least one pharmaceutically acceptable carrier.

46. A method of treating a disease or condition, comprising administering to a subject in need thereof, a therapeutically effective amount of a compound according to any one of technical solutions 1 to 44, a tautomer thereof, a solvate or stereoisomer of the compound or the tautomer, or a pharmaceutically acceptable salt of the foregoing or the pharmaceutical composition according to technical solution 45; wherein the disease or condition is selected from neuropathy, stroke, neurodegenerative disease, Parkinson's Disease, amyotrophic lateral sclerosis (AML), multiple sclerosis, Huntington's Disease, dementia with Lewy bodies, Friedreich's ataxia, hair follicle morphogenesis, diabetes, sepsis, transplant rejection, periventricular leukomalacia, ischemia reperfusion injury, blood coagulation, myocardial infarction, and kidney dysfunction.

47. A method of treating a disease or condition involving ferroptosis, comprising administering to a subject in need thereof, a therapeutically effective amount of a compound according to any one of technical solutions 1 to 44, a tautomer thereof, a solvate or stereoisomer of the compound or the tautomer, or a pharmaceutically acceptable salt of the foregoing or the pharmaceutical composition according to technical solution 45.

48. A method of modulating ferroptosis, comprising contacting a subject in need thereof with a compound according to any one of technical solutions 1 to 44, a tautomer thereof, a solvate or stereoisomer of the compound or the tautomer, or a pharmaceutically acceptable salt of the foregoing or the pharmaceutical composition according to technical solution 45.

49. A compound selected from:

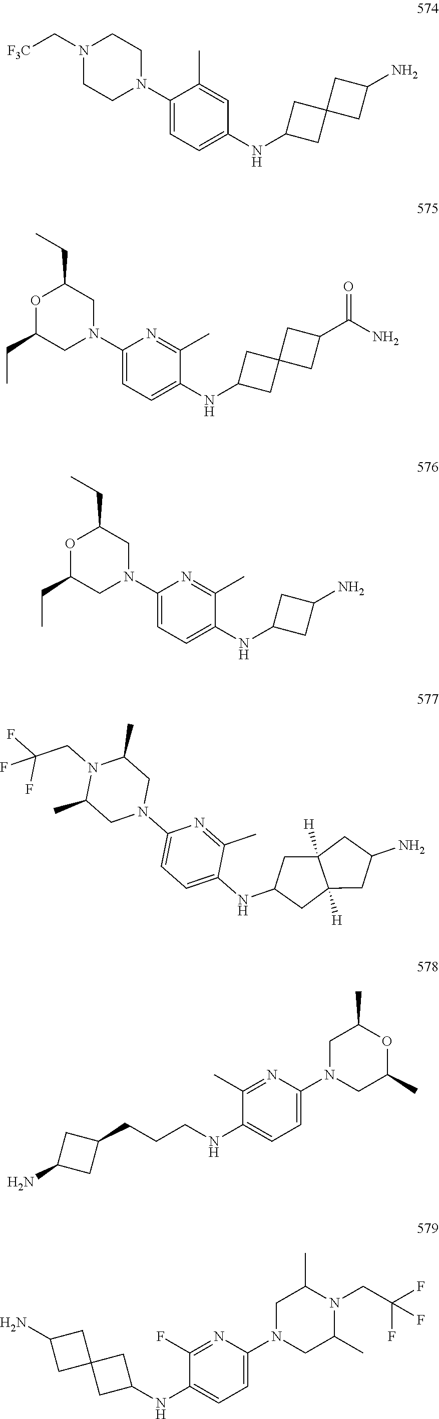 [SMILES: Cc1cc(NC2CC3(CC(N)C3)C2)ccc1N1CCN(CC(F)(F)F)CC1 ; CC[C@@H]1CN(c2ccc(NC3CC4(C3)CC(C(N)=O)C4)c(C)n2)C[C@H](CC)O1 ; CC[C@@H]1CN(c2ccc(NC3CC(N)C3)c(C)n2)C[C@H](CC)O1 ; Cc1nc(N2C[C@@H](C)N(CC(F)(F)F)[C@@H](C)C2)ccc1NC1C[C@H]2CC(N)C[C@H]2C1 ; Cc1nc(N2C[C@@H](C)O[C@@H](C)C2)ccc1NCCC[C@H]1C[C@@H](N)C1 ; CC1CN(c2ccc(NC3CC4(CC(N)C4)C3)c(F)n2)CC(C)N1CC(F)(F)F]

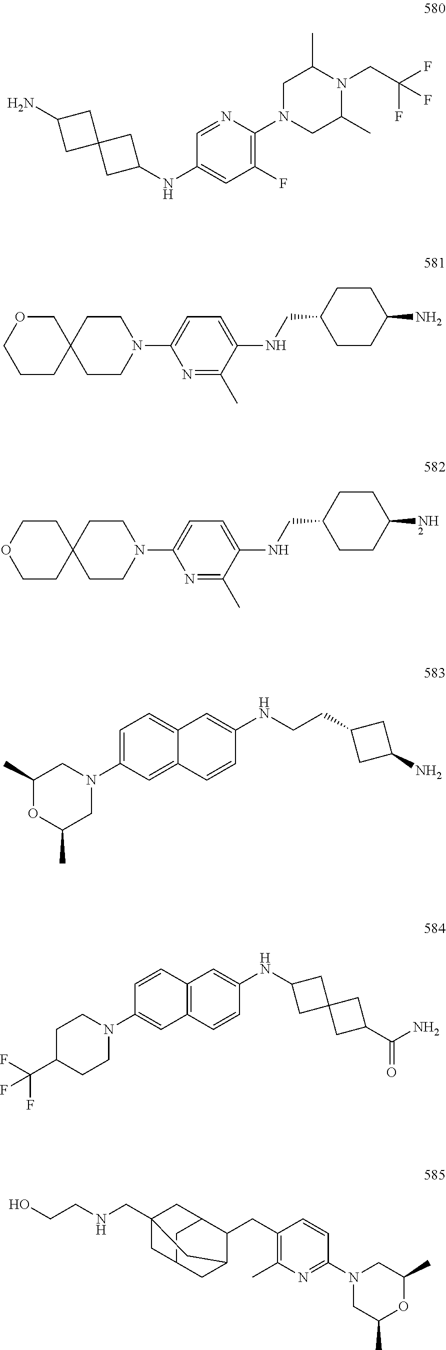 [SMILES: CC1CN(c2ncc(NC3CC4(CC(N)C4)C3)cc2F)CC(C)N1CC(F)(F)F ; Cc1nc(N2CCC3(CCCOC3)CC2)ccc1NC[C@H]1CC[C@H](N)CC1 ; Cc1nc(N2CCC3(CCOCC3)CC2)ccc1NC[C@H]1CC[C@H](N)CC1 ; C[C@@H]1CN(c2ccc3cc(NCC[C@H]4C[C@H](N)C4)ccc3c2)C[C@H](C)O1 ; NC(=O)C1CC2(CC(Nc3ccc4cc(N5CCC(C(F)(F)F)CC5)ccc4c3)C2)C1 ; Cc1nc(N2C[C@@H](C)O[C@@H](C)C2)ccc1CC1C2CC3CC1CC(CNCCO)(C3)C2]

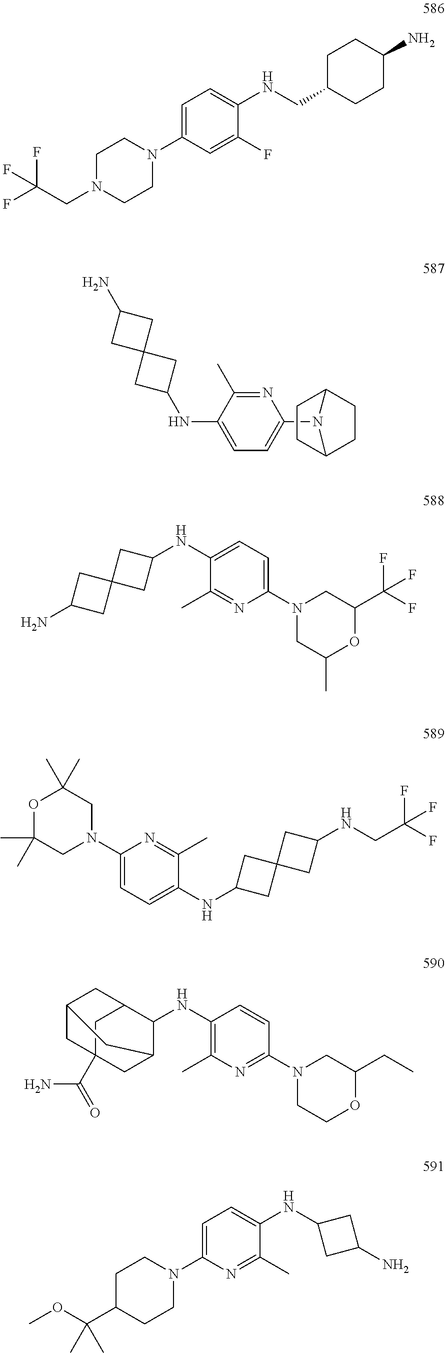

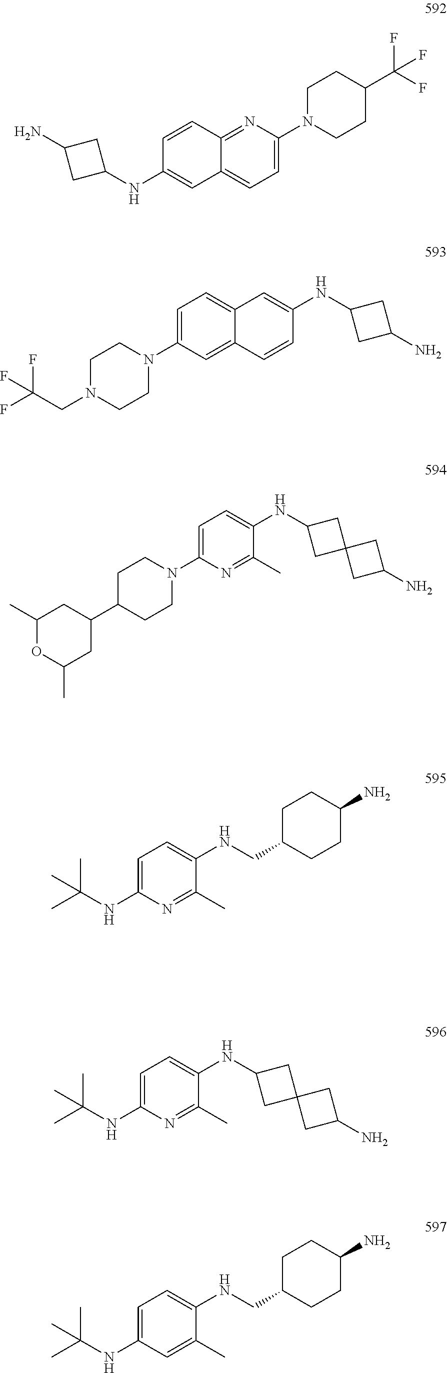 [SMILES: NC1CC(Nc2ccc3nc(N4CCC(C(F)(F)F)CC4)ccc3c2)C1 ; NC1CC(Nc2ccc3cc(N4CCN(CC(F)(F)F)CC4)ccc3c2)C1 ; Cc1nc(N2CCC(C3CC(C)OC(C)C3)CC2)ccc1NC1CC2(CC(N)C2)C1 ; Cc1nc(NC(C)(C)C)ccc1NC[C@H]1CC[C@H](N)CC1 ; Cc1nc(NC(C)(C)C)ccc1NC1CC2(CC(N)C2)C1 ; Cc1cc(NC(C)(C)C)ccc1NC[C@H]1CC[C@H](N)CC1]

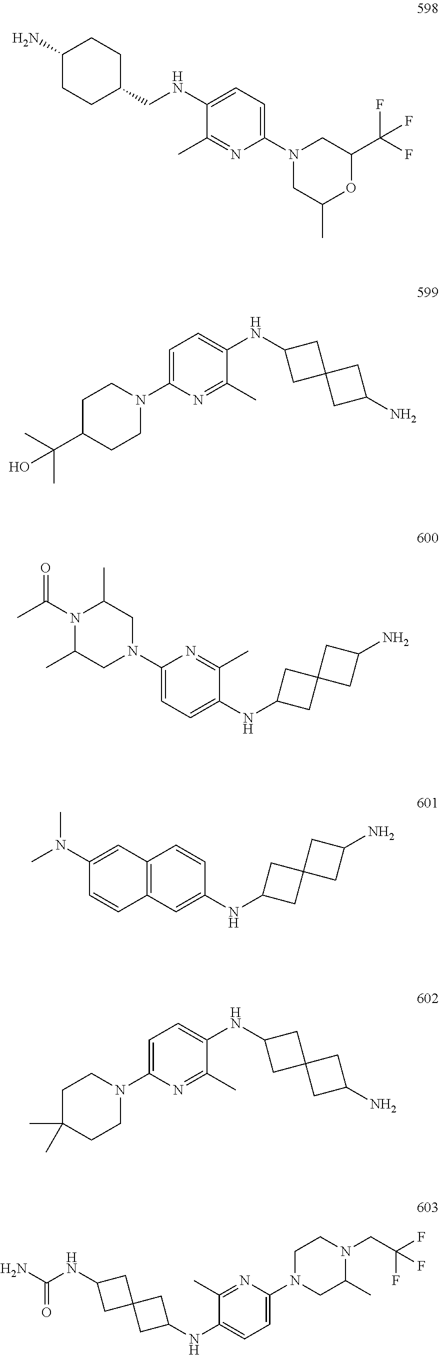 [SMILES: Cc1nc(N2CC(C)OC(C(F)(F)F)C2)ccc1NC[C@H]1CC[C@@H](N)CC1 ; Cc1nc(N2CCC(C(C)(C)O)CC2)ccc1NC1CC2(CC(N)C2)C1 ; CC(=O)N1C(C)CN(c2ccc(NC3CC4(CC(N)C4)C3)c(C)n2)CC1C ; CN(C)c1ccc2cc(NC3CC4(CC(N)C4)C3)ccc2c1 ; Cc1nc(N2CCC(C)(C)CC2)ccc1NC1CC2(CC(N)C2)C1 ; Cc1nc(N2CCN(CC(F)(F)F)C(C)C2)ccc1NC1CC2(CC(NC(N)=O)C2)C1]

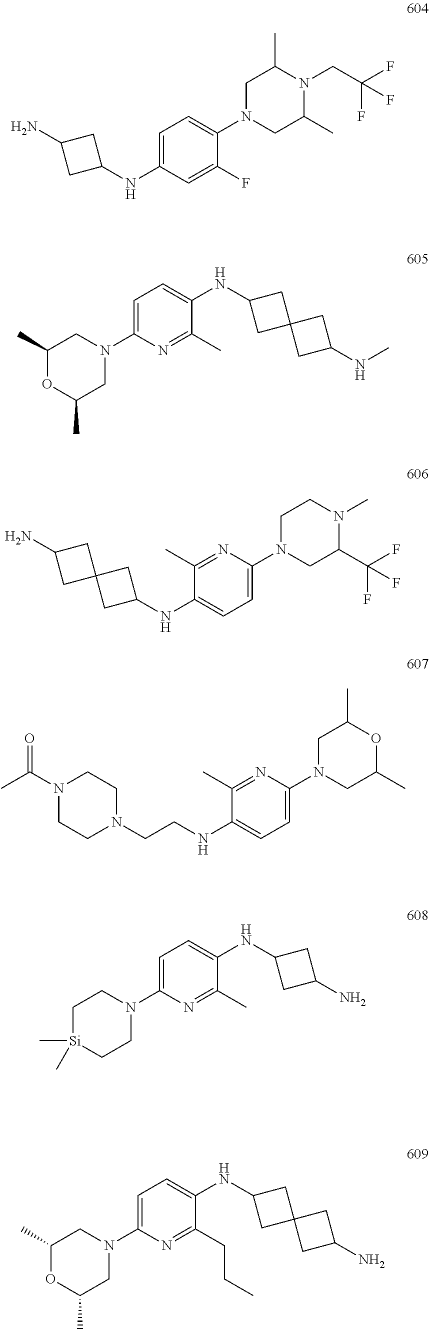

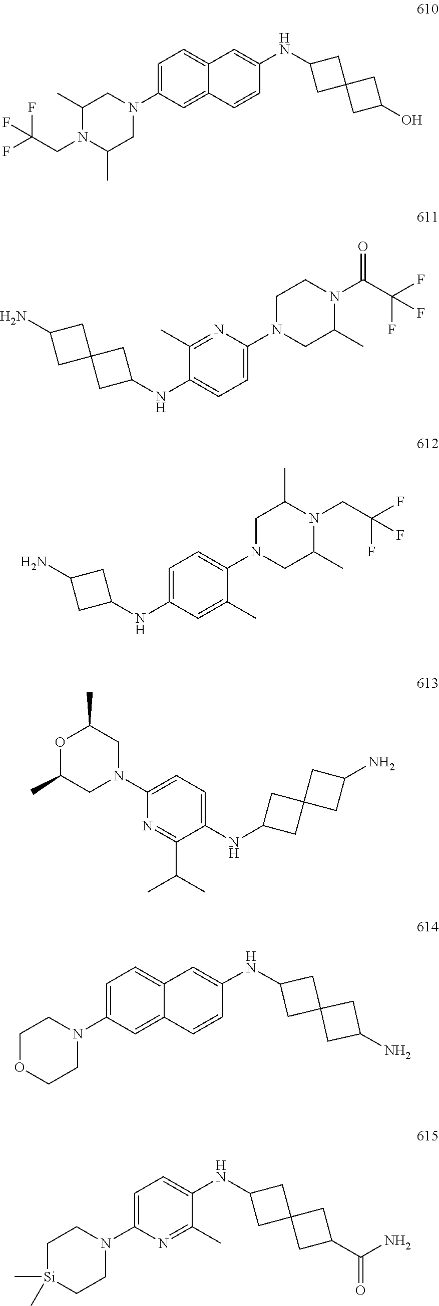 [SMILES: CC1CN(c2ccc3cc(NC4CC5(CC(O)C5)C4)ccc3c2)CC(C)N1CC(F)(F)F ; Cc1nc(N2CCN(C(=O)C(F)(F)F)C(C)C2)ccc1NC1CC2(CC(N)C2)C1 ; Cc1cc(NC2CC(N)C2)ccc1N1CC(C)N(CC(F)(F)F)C(C)C1 ; CC(C)c1nc(N2C[C@@H](C)O[C@@H](C)C2)ccc1NC1CC2(CC(N)C2)C1 ; NC1CC2(C1)CC(Nc1ccc3cc(N4CCOCC4)ccc3c1)C2 ; Cc1nc(N2CC[Si](C)(C)CC2)ccc1NC1CC2(C1)CC(C(N)=O)C2]

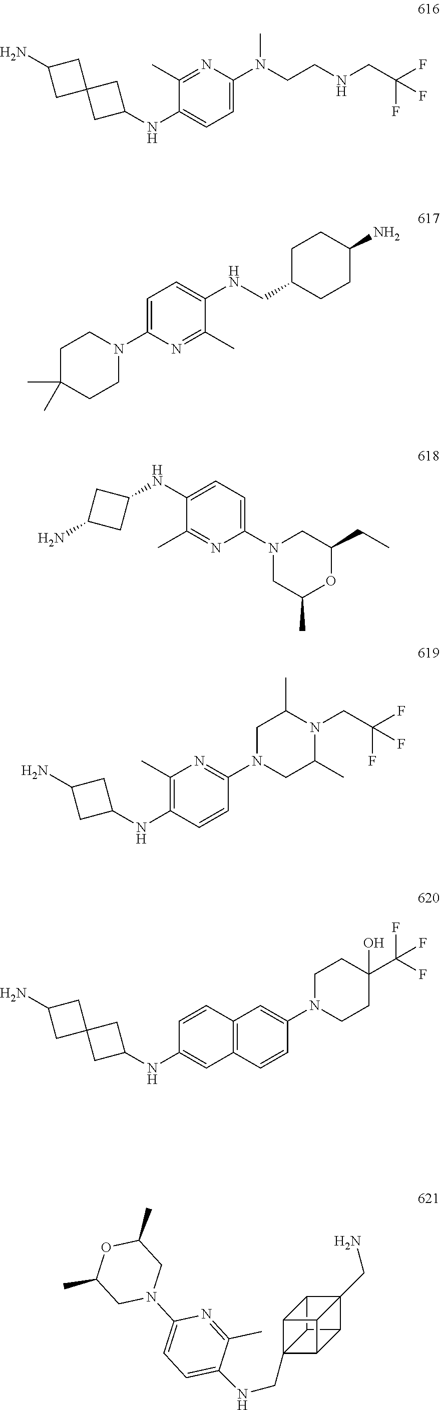

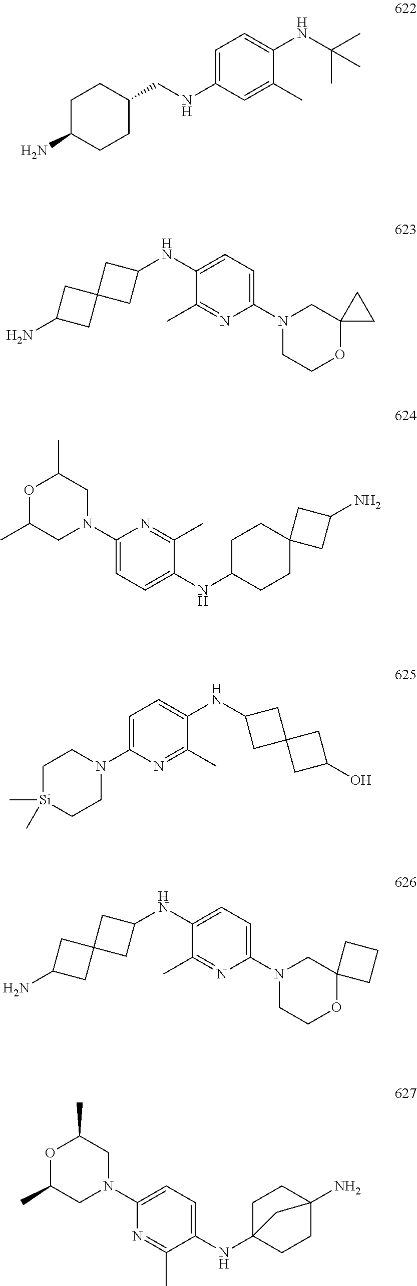

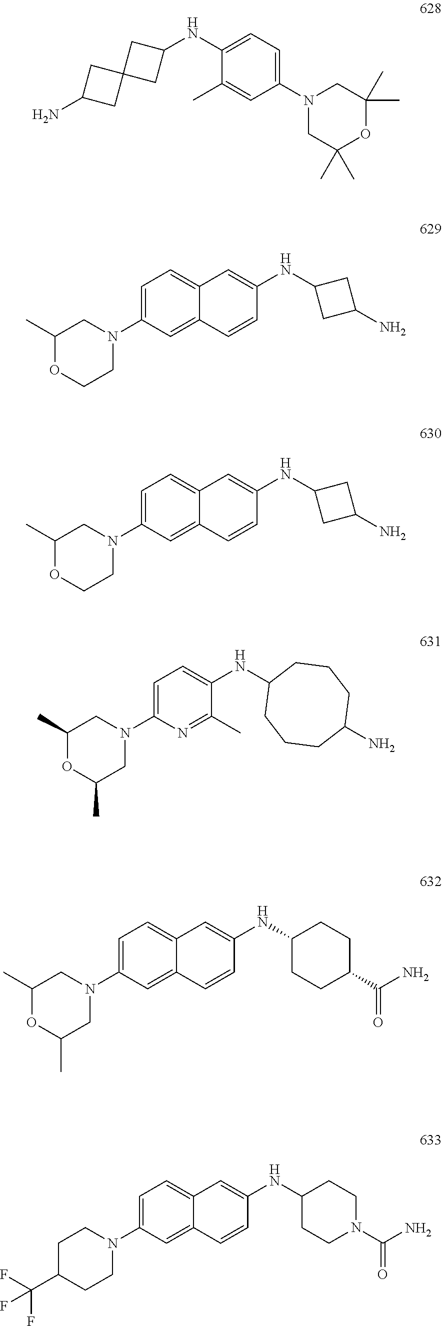

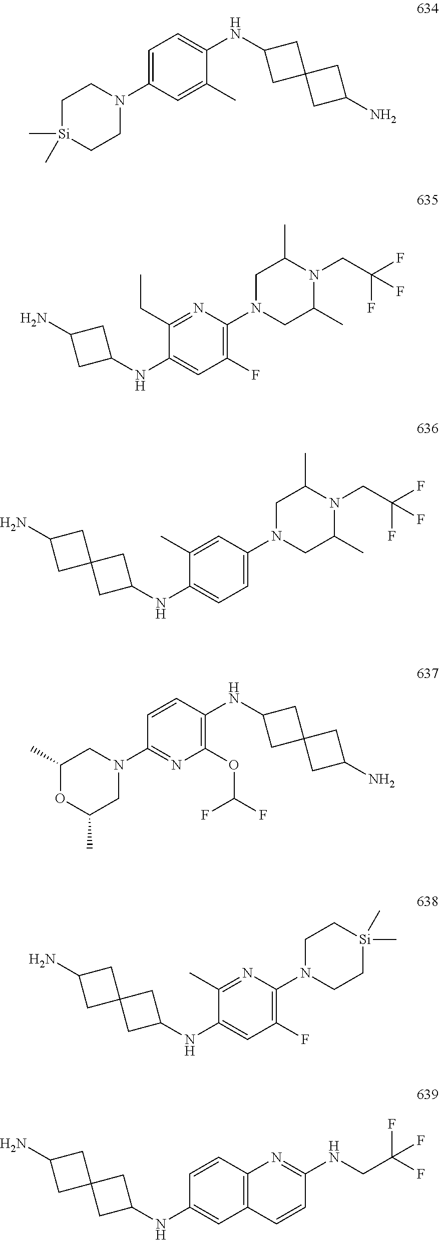 [SMILES: Cc1cc(N2CC[Si](C)(C)CC2)ccc1NC1CC2(CC(N)C2)C1 ; CCc1nc(N2CC(C)N(CC(F)(F)F)C(C)C2)c(F)cc1NC1CC(N)C1 ; Cc1cc(N2CC(C)N(CC(F)(F)F)C(C)C2)ccc1NC1CC2(CC(N)C2)C1 ; C[C@@H]1CN(c2ccc(NC3CC4(CC(N)C4)C3)c(OC(F)F)n2)C[C@H](C)O1 ; Cc1nc(N2CC[Si](C)(C)CC2)c(F)cc1NC1CC2(CC(N)C2)C1 ; NC1CC2(C1)CC(Nc1ccc3nc(NCC(F)(F)F)ccc3c1)C2]

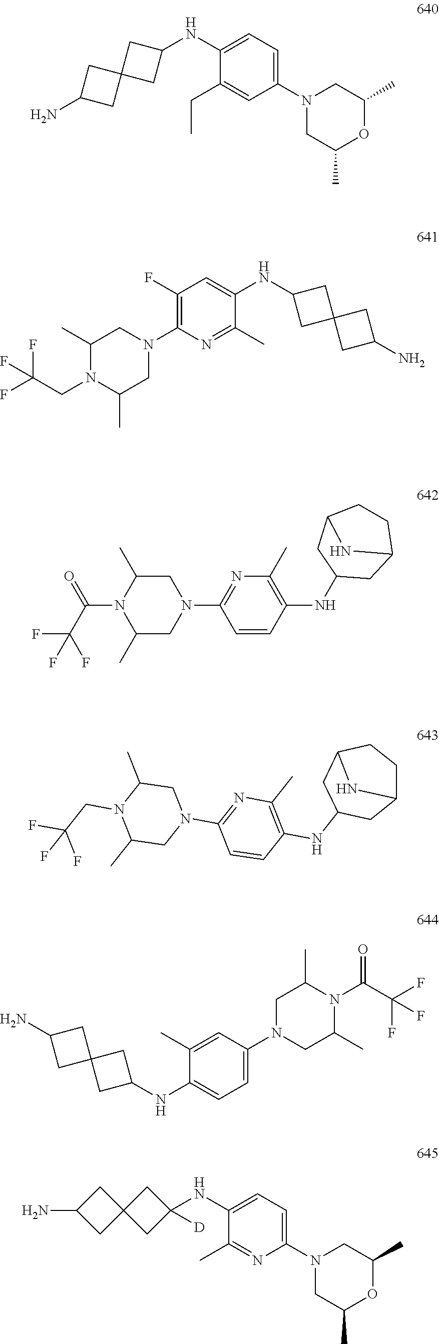 [SMILES: CCc1cc(N2C[C@@H](C)O[C@@H](C)C2)ccc1NC1CC2(CC(N)C2)C1 ; Cc1nc(N2CC(C)N(CC(F)(F)F)C(C)C2)c(F)cc1NC1CC2(CC(N)C2)C1 ; Cc1nc(N2CC(C)N(C(=O)C(F)(F)F)C(C)C2)ccc1NC1CC2CCC(C1)N2 ; Cc1nc(N2CC(C)N(CC(F)(F)F)C(C)C2)ccc1NC1CC2CCC(C1)N2 ; Cc1cc(N2CC(C)N(C(=O)C(F)(F)F)C(C)C2)ccc1NC1CC2(CC(N)C2)C1 ; [2H]C1(Nc2ccc(N3C[C@@H](C)O[C@@H](C)C3)nc2C)CC2(CC(N)C2)C1]

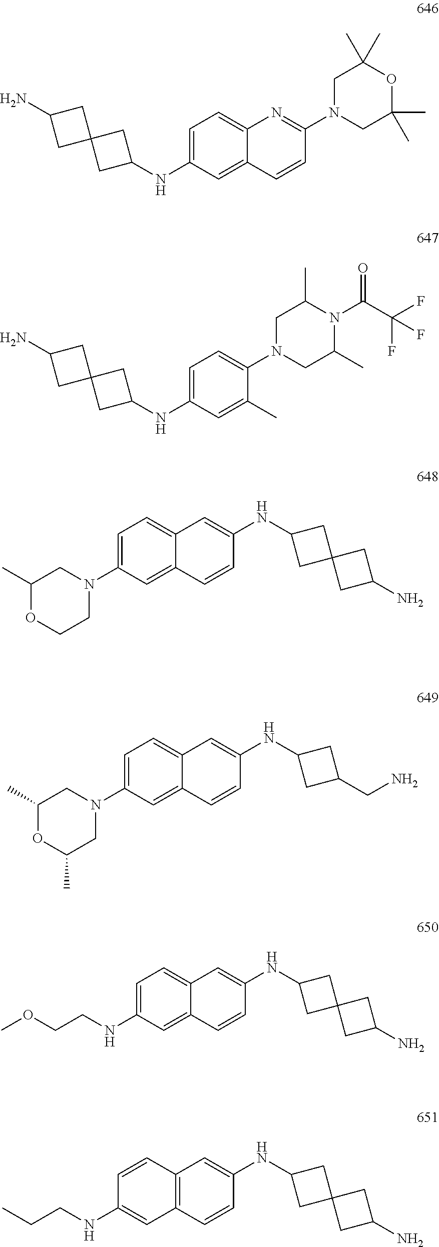

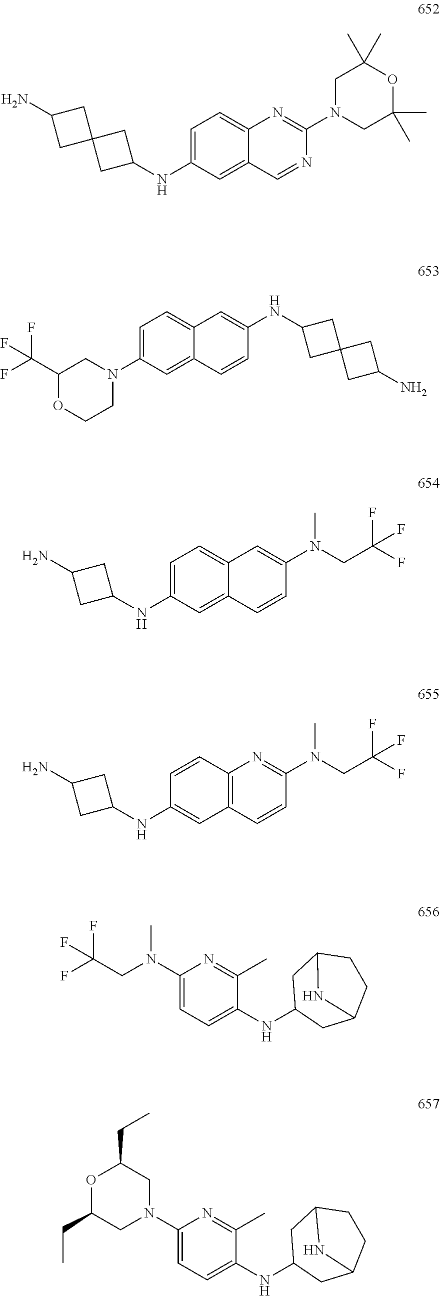 [SMILES: CC1(C)CN(c2ncc3cc(NC4CC5(CC(N)C5)C4)ccc3n2)CC(C)(C)O1 ; NC1CC2(C1)CC(Nc1ccc3cc(N4CCOC(C(F)(F)F)C4)ccc3c1)C2 ; CN(CC(F)(F)F)c1ccc2cc(NC3CC(N)C3)ccc2c1 ; CN(CC(F)(F)F)c1ccc2cc(NC3CC(N)C3)ccc2n1 ; Cc1nc(N(C)CC(F)(F)F)ccc1NC1CC2CCC(C1)N2 ; CC[C@@H]1CN(c2ccc(NC3CC4CCC(C3)N4)c(C)n2)C[C@H](CC)O1]

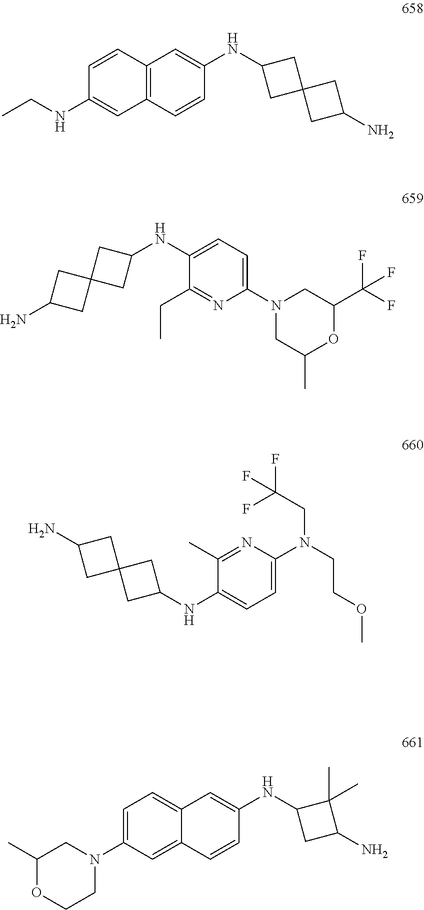

a tautomer thereof, a solvate or stereoisomer of the compound or the tautomer, or a pharmaceutically acceptable salt of the foregoing.

50. A pharmaceutical composition comprising a compound according to technical solution 49, a tautomer thereof, a solvate or stereoisomer of the compound or the tautomer, or a pharmaceutically acceptable salt of the foregoing and at least one pharmaceutically acceptable carrier.

51. A method of treating a disease or condition, comprising administering to a subject in need thereof, a therapeutically effective amount of a compound according to technical solution 49, a tautomer thereof, a solvate or stereoisomer of the compound or the tautomer, or a pharmaceutically acceptable salt of the foregoing or the pharmaceutical composition according to technical solution 50; wherein the disease or condition is selected from neuropathy, stroke, neurodegenerative disease, Parkinson's Disease, amyotrophic lateral sclerosis (AML), multiple sclerosis, Huntington's Disease, dementia with Lewy bodies, Friedreich's ataxia, hair follicle morphogenesis, diabetes, sepsis, transplant rejection, periventricular leukomalacia, ischemia reperfusion injury, blood coagulation, myocardial infarction, and kidney dysfunction.

52. A method of treating a disease or condition involving ferroptosis, comprising administering to a subject in need thereof, a therapeutically effective amount of a compound according to technical solution 49, a tautomer thereof, a solvate or stereoisomer of the compound or the tautomer, or a pharmaceutically acceptable salt of the foregoing or the pharmaceutical composition according to technical solution 50.

53. A method of modulating ferroptosis, comprising contacting a subject in need thereof with a compound according to technical solution 49, a tautomer thereof, a solvate or stereoisomer of the compound or the tautomer, or a pharmaceutically acceptable salt of the foregoing or the pharmaceutical composition according to technical solution 50.

All publications, including but not limited to disclosures and disclosure applications, cited in this specification are herein incorporated by reference as though fully set forth. If certain content of a publication cited herein contradicts or is inconsistent with the present disclosure, the present disclosure controls.

One skilled in the art will readily recognize from the disclosure and claims that various changes, modifications, and variations can be made therein without departing from the spirit and scope of the disclosure as defined in the following claims.

1. A compound of the following structural Formula I: 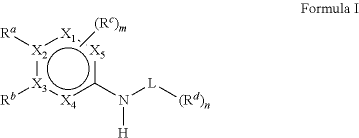
a tautomer thereof, a solvate or stereoisomer of the compound or the tautomer, or a pharmaceutically acceptable salt of the foregoing, wherein:
L is cycloalkyl, heterocyclyl, or C;
X₁, X₂, X₃, X₄, and X₅ are each independently C or N;
R^{a} is absent or selected from H, halogen, CN, optionally substituted heteroatom, optionally substituted alkyl, optionally substituted alkenyl, optionally substituted alkynyl, optionally substituted acyl, optionally substituted cycloalkyl, optionally substituted heterocyclyl, optionally substituted aryl, and optionally substituted heteroaryl;
R^{b} is absent or selected from H, halogen, CN, optionally substituted heteroatom, optionally substituted alkyl, optionally substituted cycloalkyl, and optionally substituted heterocyclyl;
R^{a} and R^{b} may join together to form an optionally substituted 3- to 10-membered carbocyclic, heterocyclic, aromatic, or heteroaromatic ring;
R^{c}, for each occurrence, is independently selected from halogen, optionally substituted heteroatom, and optionally substituted alkyl;
R^{b} and one of R^{c} may join together to form an optionally substituted 3- to 10-membered carbocyclic, heterocyclic, aromatic, or heteroaromatic ring;
R^{d}, for each occurrence, is independently selected from halogen, CN, optionally substituted heteroatom, optionally substituted alkyl, optionally substituted alkenyl, optionally substituted alkynyl, optionally substituted acyl, optionally substituted cycloalkyl, optionally substituted heterocyclyl, optionally substituted aryl, and optionally substituted heteroaryl; and
m and n are each an integer independently selected from 0, 1, 2, and 3.

2. The compound of claim 1, wherein the compound has one of the following structural Formulae IIa to IIh: 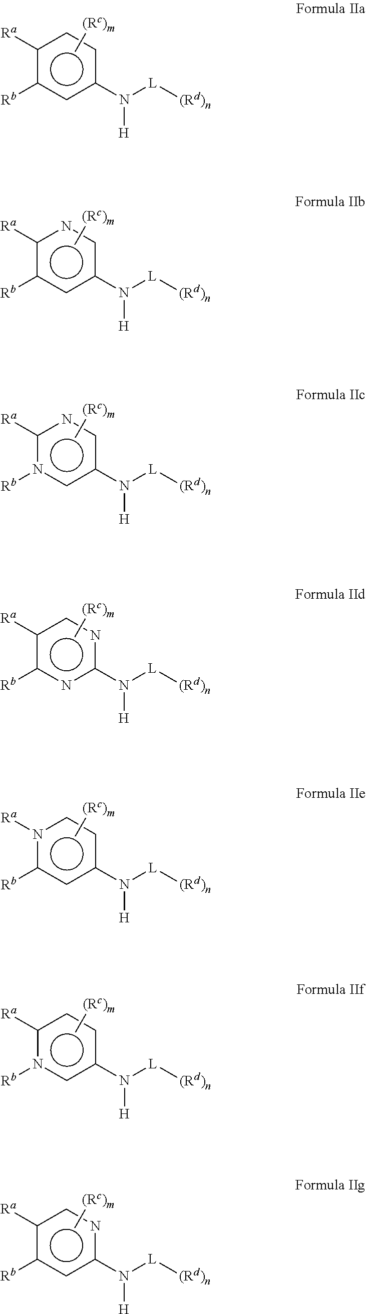 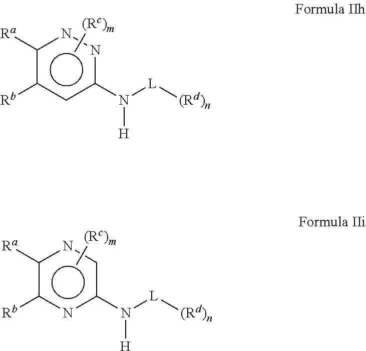
a tautomer thereof, a solvate or stereoisomer of the compound or the tautomer, or a pharmaceutically acceptable salt of the foregoing.

3. The compound of claim 1, wherein the compound has one of the following structural Formulae IIj: 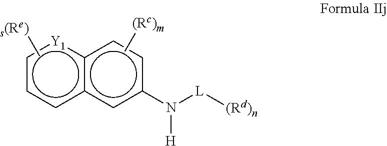
a tautomer thereof, a solvate or stereoisomer of the compound or the tautomer, or a pharmaceutically acceptable salt of the foregoing, wherein Y₁ is N or C; R^{e}, for each occurrence, is independently selected from optionally substituted cycloalkyl, optionally substituted heterocyclyl, optionally substituted aryl, and optionally substituted heteroaryl; and s is an integer selected from 0, 1, 2, and 3.

4. The compound of claim 1, wherein the compound has one of the following structural Formulae IIIa and IIIb: 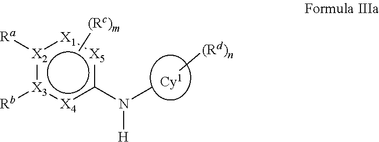
wherein Cy¹ is 3- to 10-membered cycloalkyl or 3- to 10-membered heterocyclyl; 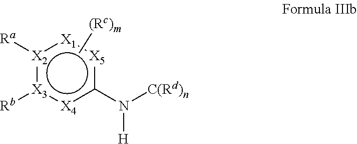
a tautomer thereof, a solvate or stereoisomer of the compound or the tautomer, or a pharmaceutically acceptable salt of the foregoing.

5. The compound of claim 1, wherein
the compound has one of the following structural Formulae IVa to IVf: 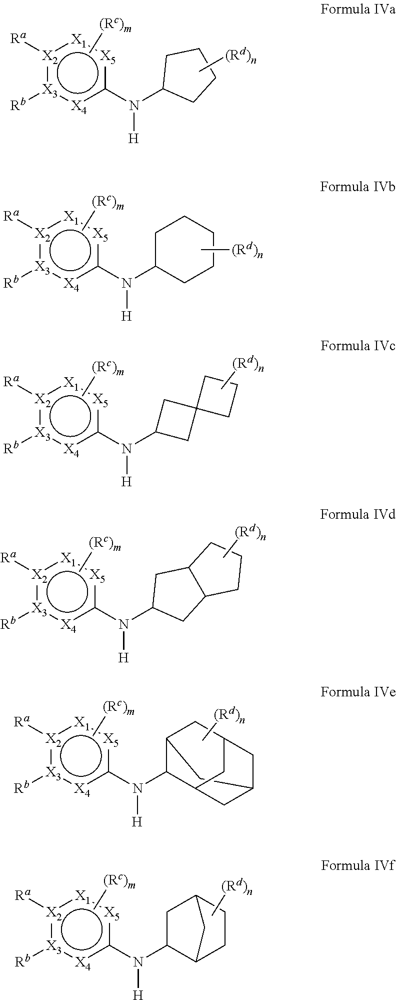
a tautomer thereof, a solvate or stereoisomer of the compound or the tautomer, or a pharmaceutically acceptable salt of the foregoing.

6. The compound of claim 1, wherein
the compound has one of the following structural Formula Va and Formula VIa: 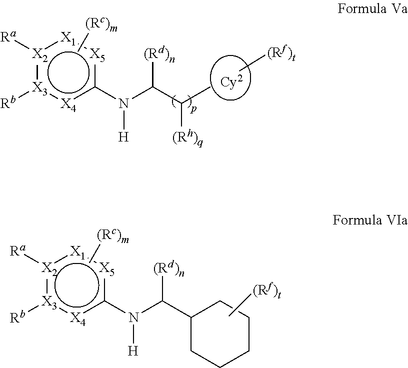
a tautomer thereof, a solvate or stereoisomer of the compound or the tautomer, or a pharmaceutically acceptable salt of the foregoing, wherein Cy² is 3- to 10-membered cycloalkyl or 3- to 10-membered heterocyclyl; R^{f}, for each occurrence, is independently selected from halogen, CN, optionally substituted heteroatom, optionally substituted alkyl, optionally substituted alkenyl, optionally substituted alkynyl, optionally substituted acyl, optionally substituted cycloalkyl, optionally substituted heterocyclyl, optionally substituted aryl, and optionally substituted heteroaryl; R^{h}, for each occurrence, is independently selected from halogen, CN, and optionally substituted C₁ to C₆ alkyl; n is an integer selected from 0, 1, and 2; p is an integer selected from 0, 1, 2, 3, 4, and 5; q is an integer selected from 0, 1, and 2; and t is an integer independently selected from 0, 1, 2, and 3.

7. The compound of claim 1, wherein the compound has one of the following structural Formulae VIIa-VIIe: 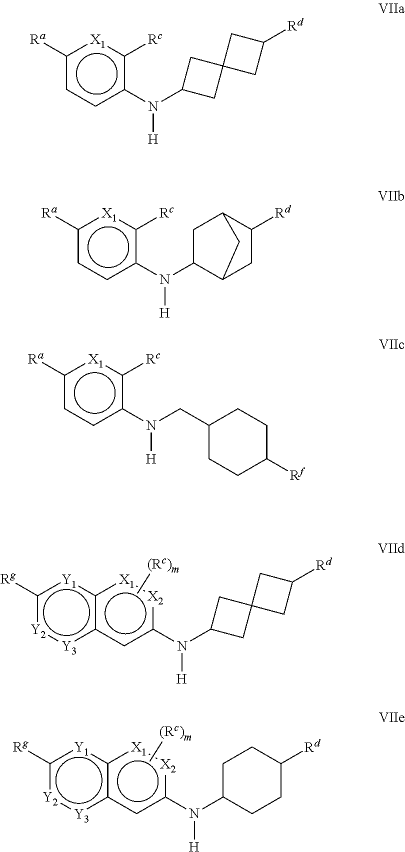
a tautomer thereof, a solvate or stereoisomer of the compound or the tautomer, or a pharmaceutically acceptable salt of the foregoing, wherein:
X₁ and X₂ are independently C or N;
Y₁, Y₂, and Y₃ are independently C or N;
R^{a} is selected from 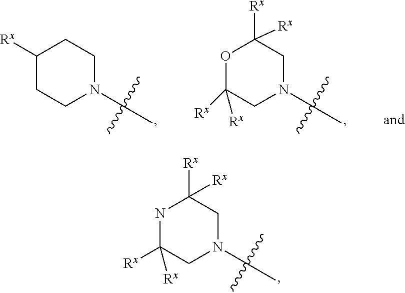
wherein R^{x}, for each occurrence, is independently selected from H, CF₃, —CH₃, and —CH₂CH₃;
R^{c} is selected from halogen, C₁ to C₂ alkyl, and C₁ to C₂ alkoxy;
R^{d} is selected from NH₂ and —C(═O) NH₂;
R^{f} is selected from NH₂ and —C(═O)NH₂; and
m is 0 or 1.

8. A compound selected from: 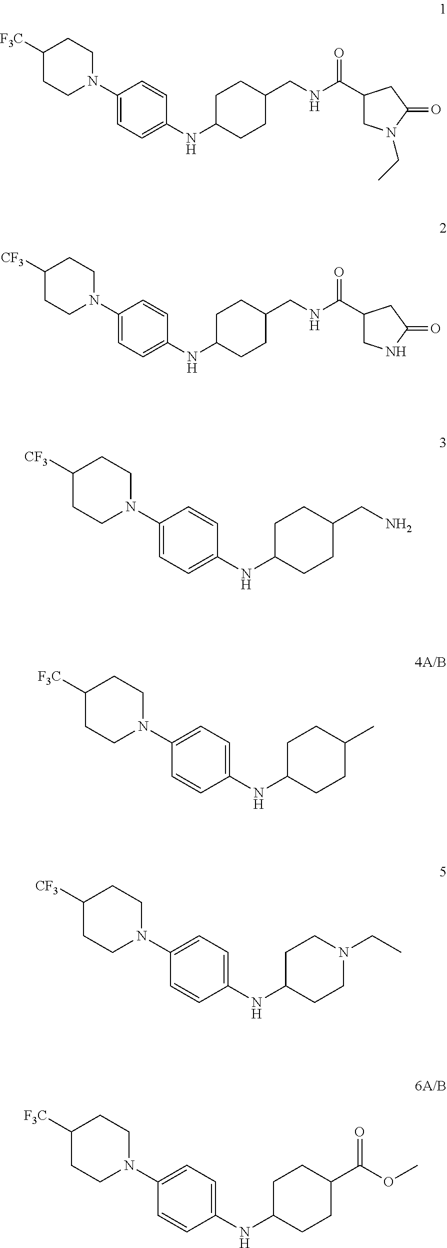 [SMILES: CCN1CC(C(=O)NCC2CCC(Nc3ccc(N4CCC(C(F)(F)F)CC4)cc3)CC2)CC1=O ; O=C1CC(C(=O)NCC2CCC(Nc3ccc(N4CCC(C(F)(F)F)CC4)cc3)CC2)CN1 ; NCC1CCC(Nc2ccc(N3CCC(C(F)(F)F)CC3)cc2)CC1 ; CC1CCC(Nc2ccc(N3CCC(C(F)(F)F)CC3)cc2)CC1 ; CCN1CCC(Nc2ccc(N3CCC(C(F)(F)F)CC3)cc2)CC1 ; COC(=O)C1CCC(Nc2ccc(N3CCC(C(F)(F)F)CC3)cc2)CC1] 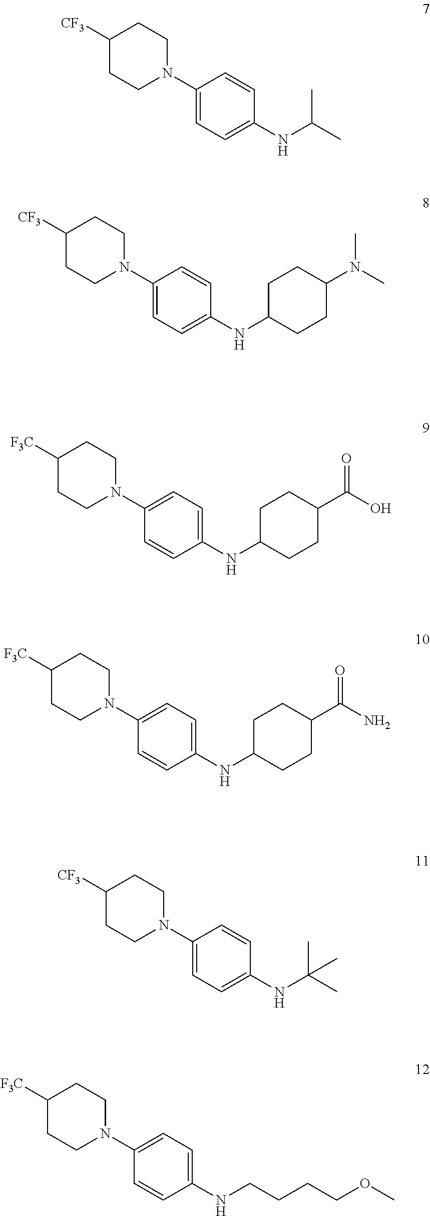 [SMILES: CC(C)Nc1ccc(N2CCC(C(F)(F)F)CC2)cc1 ; CN(C)C1CCC(Nc2ccc(N3CCC(C(F)(F)F)CC3)cc2)CC1 ; O=C(O)C1CCC(Nc2ccc(N3CCC(C(F)(F)F)CC3)cc2)CC1 ; NC(=O)C1CCC(Nc2ccc(N3CCC(C(F)(F)F)CC3)cc2)CC1 ; CC(C)(C)Nc1ccc(N2CCC(C(F)(F)F)CC2)cc1 ; COCCCCNc1ccc(N2CCC(C(F)(F)F)CC2)cc1] 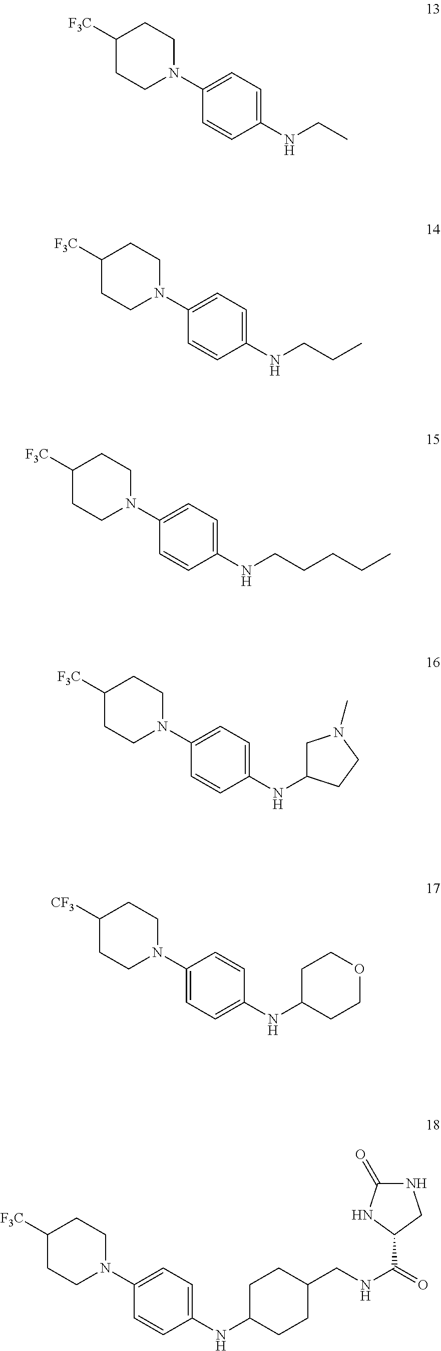 [SMILES: CCNc1ccc(N2CCC(C(F)(F)F)CC2)cc1 ; CCCNc1ccc(N2CCC(C(F)(F)F)CC2)cc1 ; CCCCCNc1ccc(N2CCC(C(F)(F)F)CC2)cc1 ; CN1CCC(Nc2ccc(N3CCC(C(F)(F)F)CC3)cc2)C1 ; FC(F)(F)C1CCN(c2ccc(NC3CCOCC3)cc2)CC1 ; O=C1NC[C@H](C(=O)NCC2CCC(Nc3ccc(N4CCC(C(F)(F)F)CC4)cc3)CC2)N1] 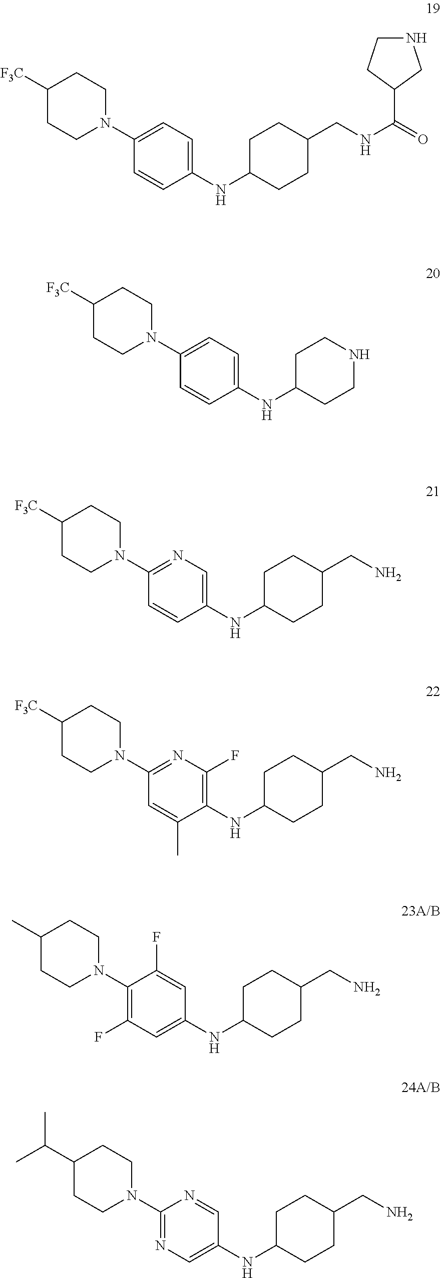 [SMILES: O=C(NCC1CCC(Nc2ccc(N3CCC(C(F)(F)F)CC3)cc2)CC1)C1CCNC1 ; FC(F)(F)C1CCN(c2ccc(NC3CCNCC3)cc2)CC1 ; NCC1CCC(Nc2ccc(N3CCC(C(F)(F)F)CC3)nc2)CC1 ; Cc1cc(N2CCC(C(F)(F)F)CC2)nc(F)c1NC1CCC(CN)CC1 ; CC1CCN(c2c(F)cc(NC3CCC(CN)CC3)cc2F)CC1 ; CC(C)C1CCN(c2ncc(NC3CCC(CN)CC3)cn2)CC1] 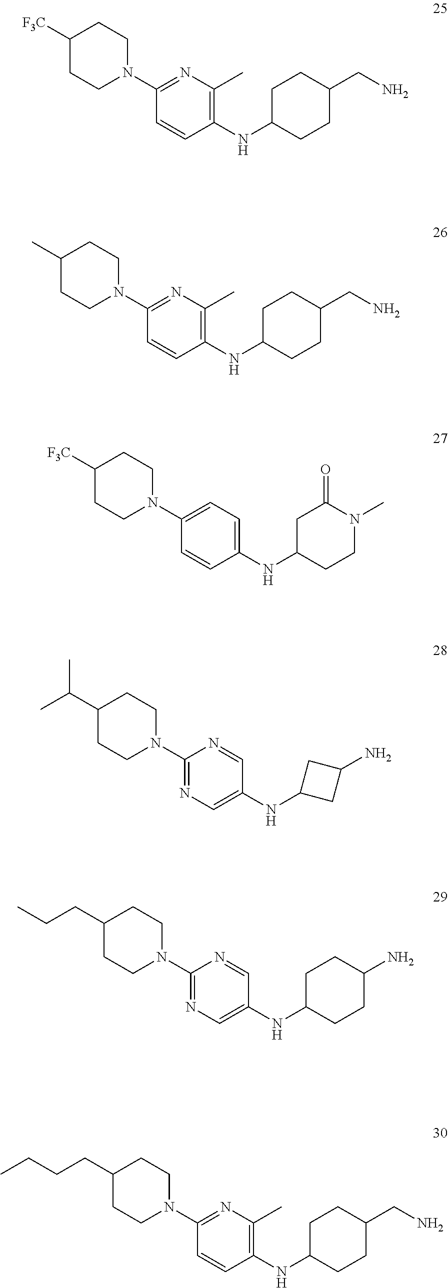 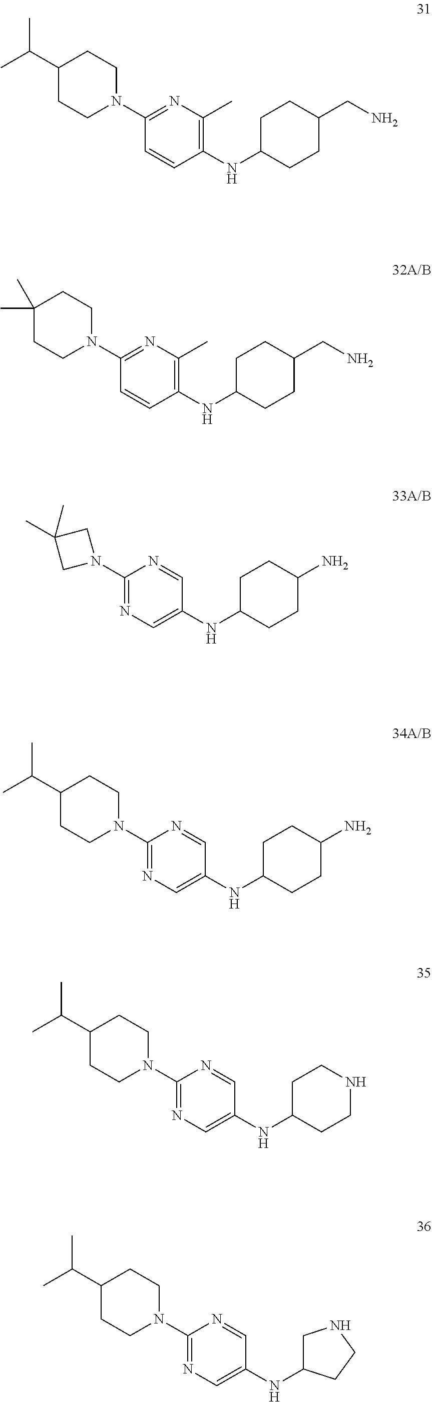 [SMILES: Cc1nc(N2CCC(C(C)C)CC2)ccc1NC1CCC(CN)CC1 ; Cc1nc(N2CCC(C)(C)CC2)ccc1NC1CCC(CN)CC1 ; CC1(C)CN(c2ncc(NC3CCC(N)CC3)cn2)C1 ; CC(C)C1CCN(c2ncc(NC3CCC(N)CC3)cn2)CC1 ; CC(C)C1CCN(c2ncc(NC3CCNCC3)cn2)CC1 ; CC(C)C1CCN(c2ncc(NC3CCNC3)cn2)CC1] 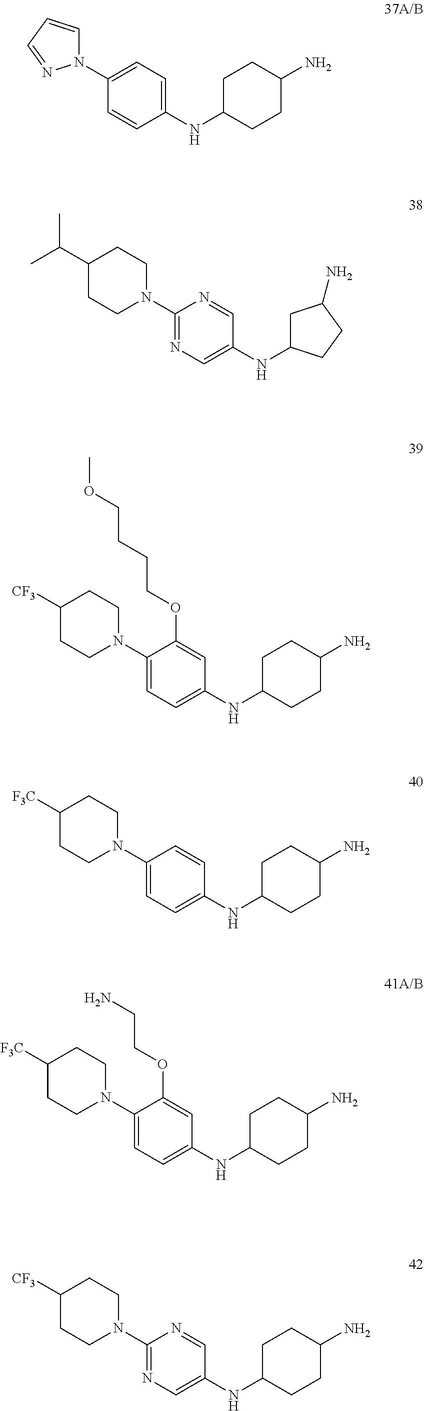 [SMILES: NC1CCC(Nc2ccc(-n3cccn3)cc2)CC1 ; CC(C)C1CCN(c2ncc(NC3CCC(N)C3)cn2)CC1 ; COCCCCOc1cc(NC2CCC(N)CC2)ccc1N1CCC(C(F)(F)F)CC1 ; NC1CCC(Nc2ccc(N3CCC(C(F)(F)F)CC3)cc2)CC1 ; NCCOc1cc(NC2CCC(N)CC2)ccc1N1CCC(C(F)(F)F)CC1 ; NC1CCC(Nc2cnc(N3CCC(C(F)(F)F)CC3)nc2)CC1] 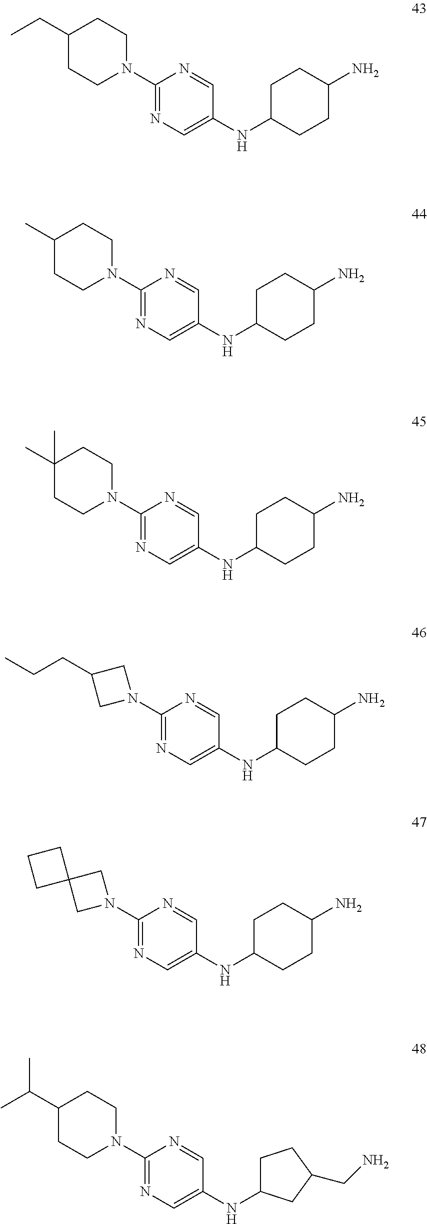 [SMILES: CCC1CCN(c2ncc(NC3CCC(N)CC3)cn2)CC1 ; CC1CCN(c2ncc(NC3CCC(N)CC3)cn2)CC1 ; CC1(C)CCN(c2ncc(NC3CCC(N)CC3)cn2)CC1 ; CCCC1CN(c2ncc(NC3CCC(N)CC3)cn2)C1 ; NC1CCC(Nc2cnc(N3CC4(CCC4)C3)nc2)CC1 ; CC(C)C1CCN(c2ncc(NC3CCC(CN)C3)cn2)CC1] 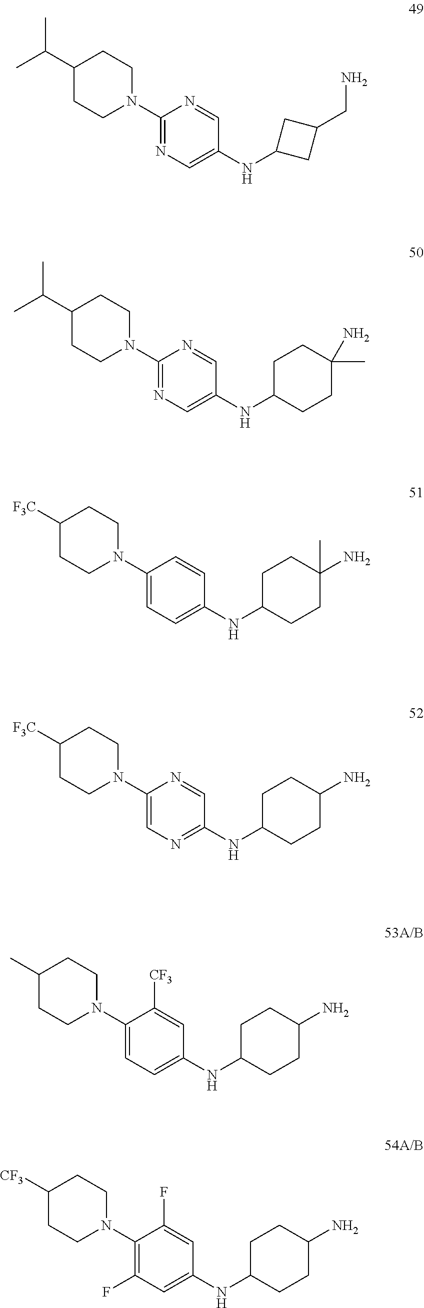 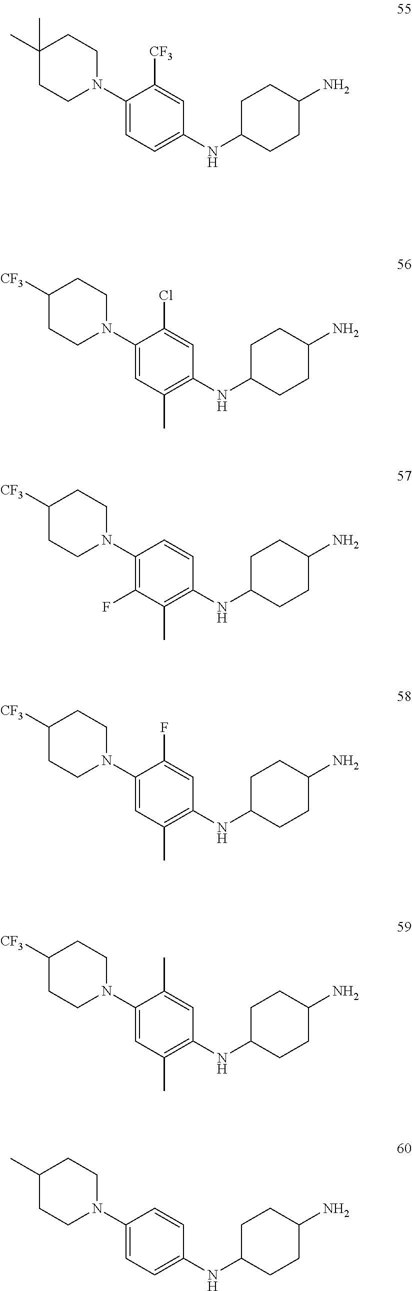 [SMILES: CC1(C)CCN(c2ccc(NC3CCC(N)CC3)cc2C(F)(F)F)CC1 ; Cc1cc(N2CCC(C(F)(F)F)CC2)c(Cl)cc1NC1CCC(N)CC1 ; Cc1c(NC2CCC(N)CC2)ccc(N2CCC(C(F)(F)F)CC2)c1F ; Cc1cc(N2CCC(C(F)(F)F)CC2)c(F)cc1NC1CCC(N)CC1 ; Cc1cc(N2CCC(C(F)(F)F)CC2)c(C)cc1NC1CCC(N)CC1 ; CC1CCN(c2ccc(NC3CCC(N)CC3)cc2)CC1] 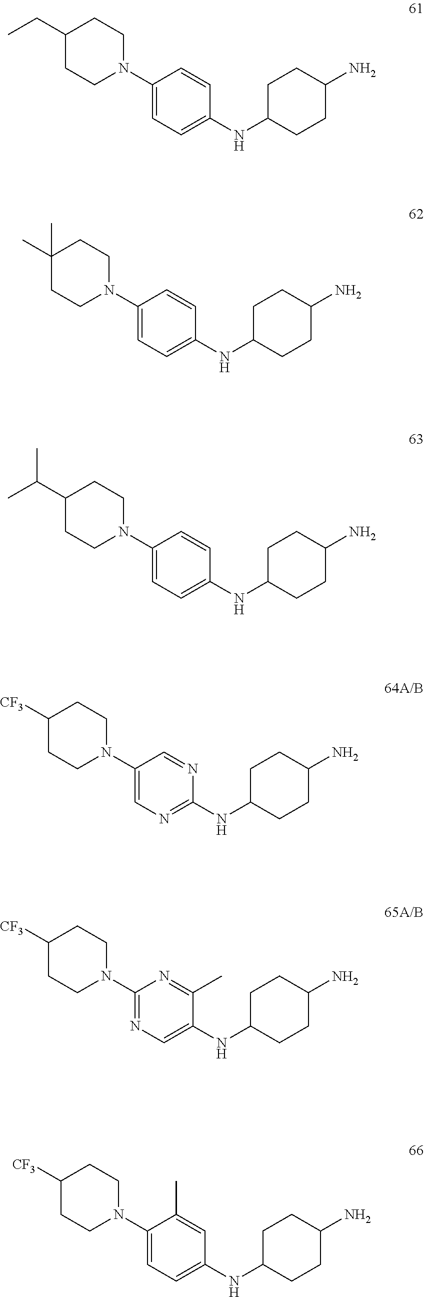 [SMILES: CCC1CCN(c2ccc(NC3CCC(N)CC3)cc2)CC1 ; CC1(C)CCN(c2ccc(NC3CCC(N)CC3)cc2)CC1 ; CC(C)C1CCN(c2ccc(NC3CCC(N)CC3)cc2)CC1 ; NC1CCC(Nc2ncc(N3CCC(C(F)(F)F)CC3)cn2)CC1 ; Cc1nc(N2CCC(C(F)(F)F)CC2)ncc1NC1CCC(N)CC1 ; Cc1cc(NC2CCC(N)CC2)ccc1N1CCC(C(F)(F)F)CC1] 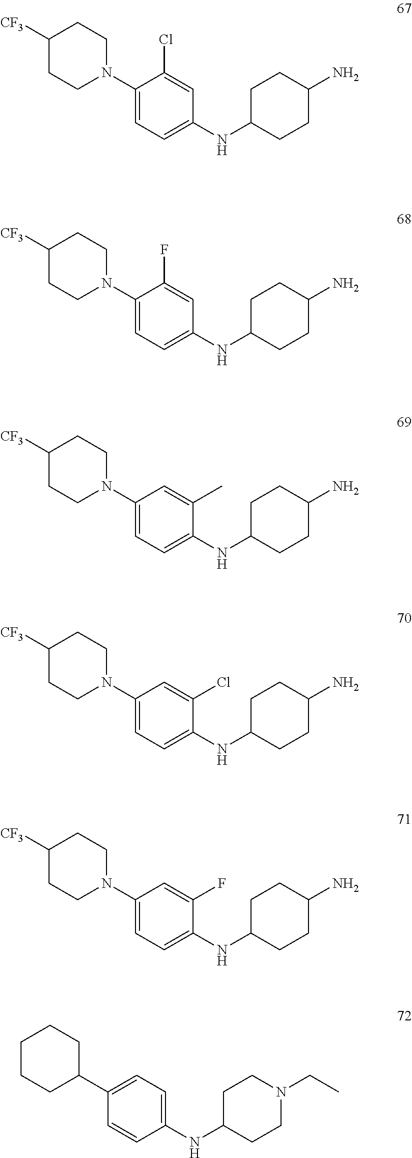 [SMILES: NC1CCC(Nc2ccc(N3CCC(C(F)(F)F)CC3)c(Cl)c2)CC1 ; NC1CCC(Nc2ccc(N3CCC(C(F)(F)F)CC3)c(F)c2)CC1 ; Cc1cc(N2CCC(C(F)(F)F)CC2)ccc1NC1CCC(N)CC1 ; NC1CCC(Nc2ccc(N3CCC(C(F)(F)F)CC3)cc2Cl)CC1 ; NC1CCC(Nc2ccc(N3CCC(C(F)(F)F)CC3)cc2F)CC1 ; CCN1CCC(Nc2ccc(C3CCCCC3)cc2)CC1] 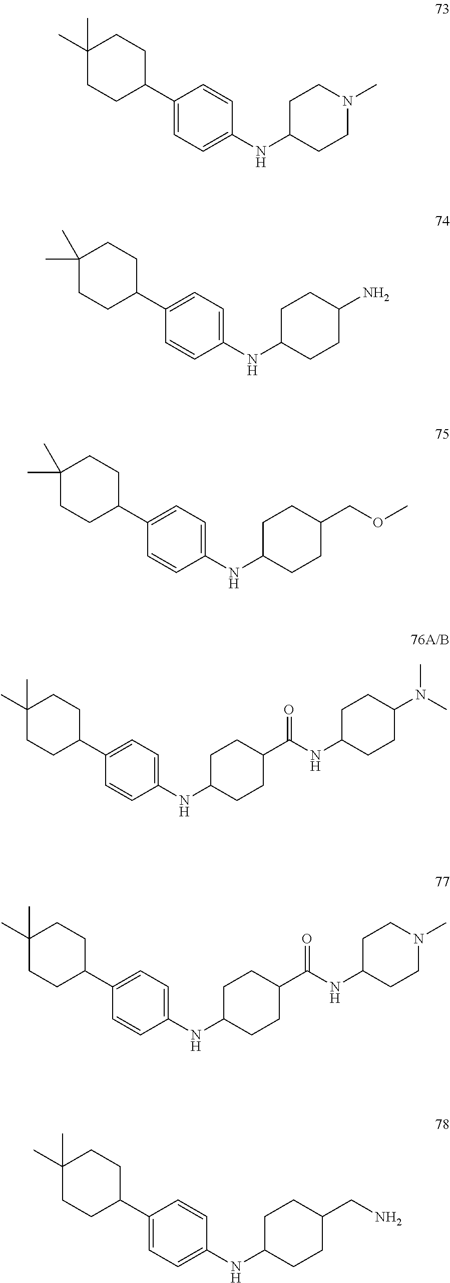 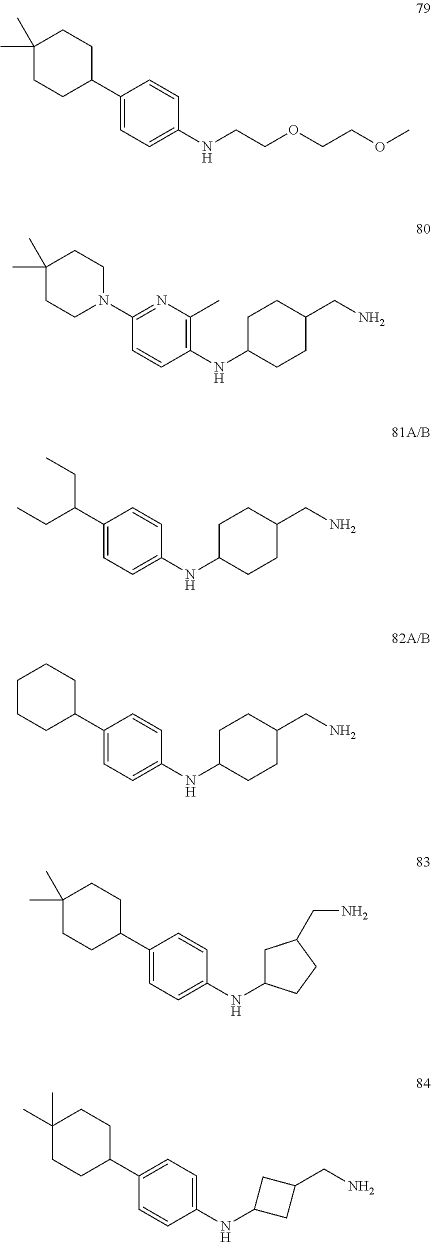 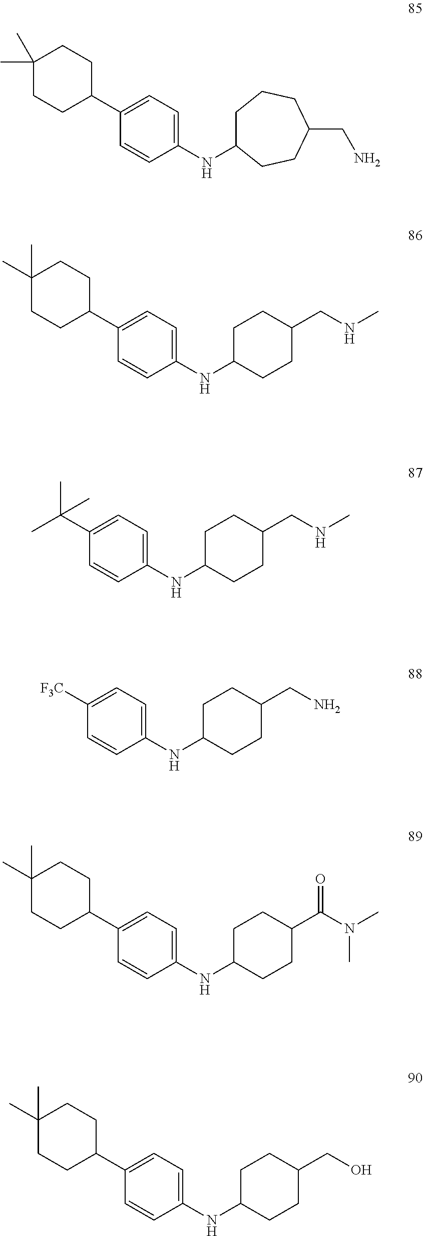 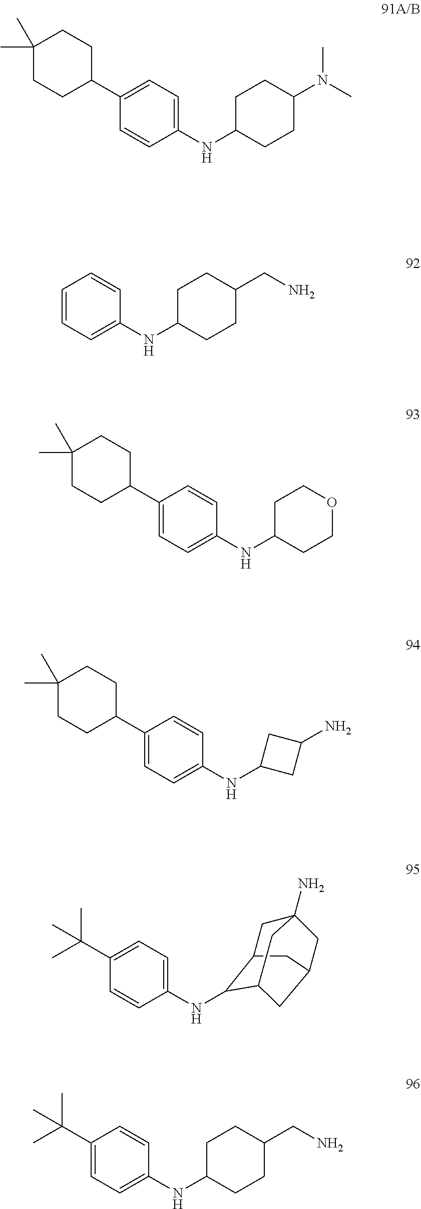 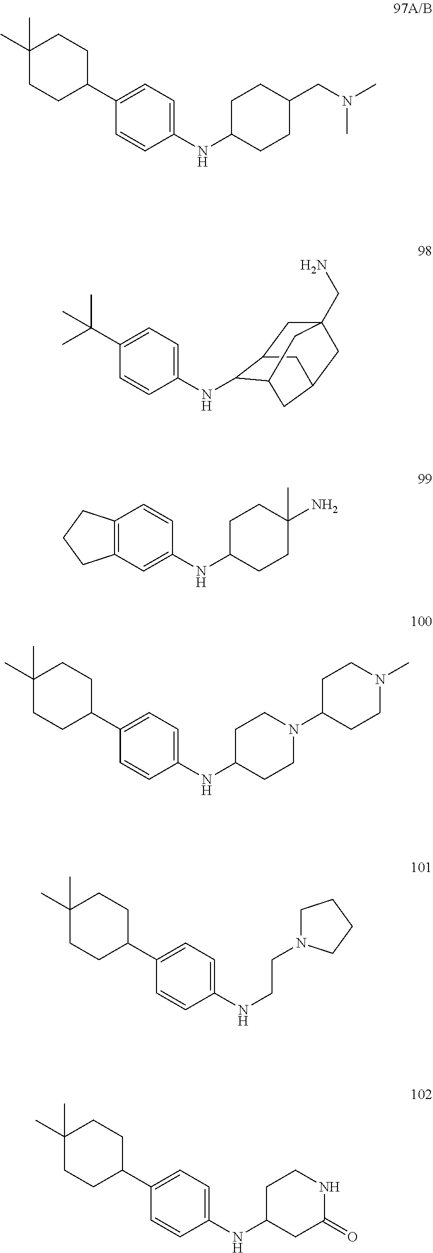 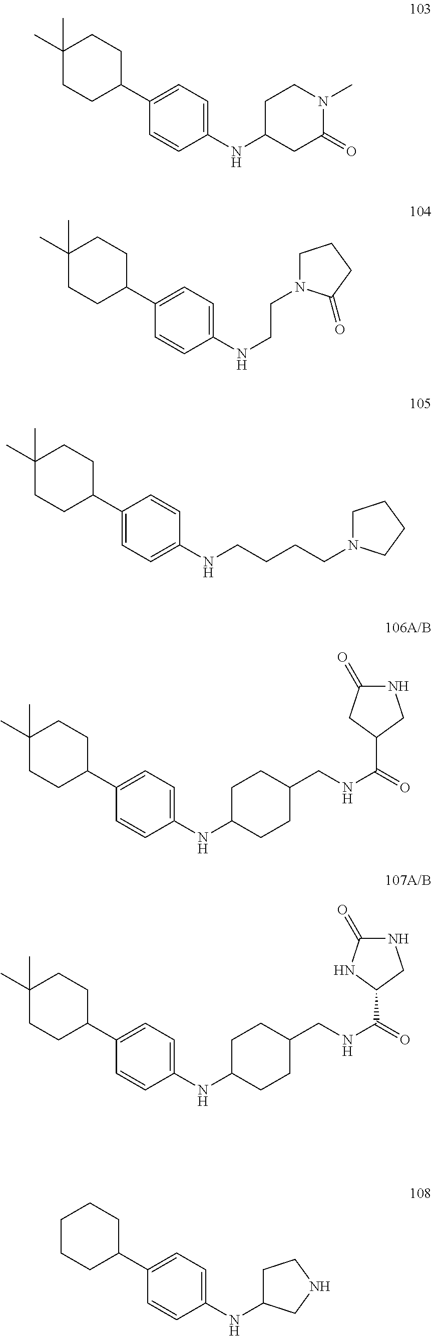 [SMILES: CN1CCC(Nc2ccc(C3CCC(C)(C)CC3)cc2)CC1=O ; CC1(C)CCC(c2ccc(NCCN3CCCC3=O)cc2)CC1 ; CC1(C)CCC(c2ccc(NCCCCN3CCCC3)cc2)CC1 ; CC1(C)CCC(c2ccc(NC3CCC(CNC(=O)C4CNC(=O)C4)CC3)cc2)CC1 ; CC1(C)CCC(c2ccc(NC3CCC(CNC(=O)[C@H]4CNC(=O)N4)CC3)cc2)CC1 ; c1cc(C2CCCCC2)ccc1NC1CCNC1] 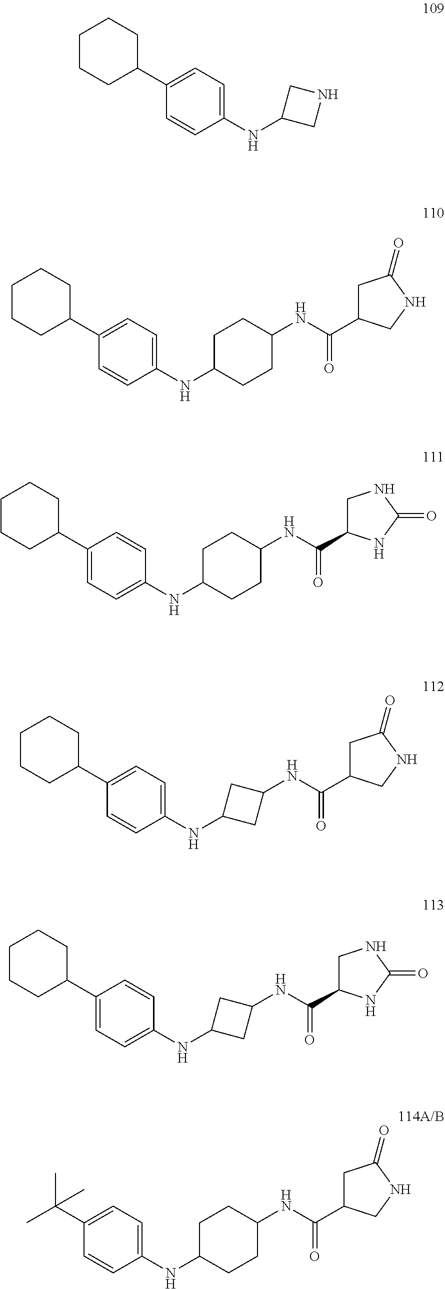 [SMILES: c1cc(C2CCCCC2)ccc1NC1CNC1 ; O=C1CC(C(=O)NC2CCC(Nc3ccc(C4CCCCC4)cc3)CC2)CN1 ; O=C1NC[C@H](C(=O)NC2CCC(Nc3ccc(C4CCCCC4)cc3)CC2)N1 ; O=C1CC(C(=O)NC2CC(Nc3ccc(C4CCCCC4)cc3)C2)CN1 ; O=C1NC[C@H](C(=O)NC2CC(Nc3ccc(C4CCCCC4)cc3)C2)N1 ; CC(C)(C)c1ccc(NC2CCC(NC(=O)C3CNC(=O)C3)CC2)cc1] 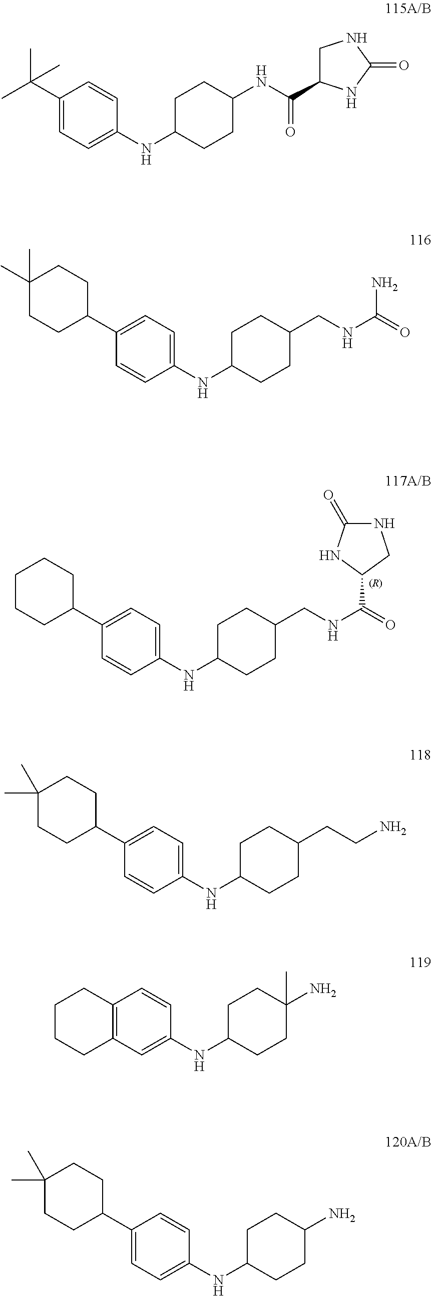 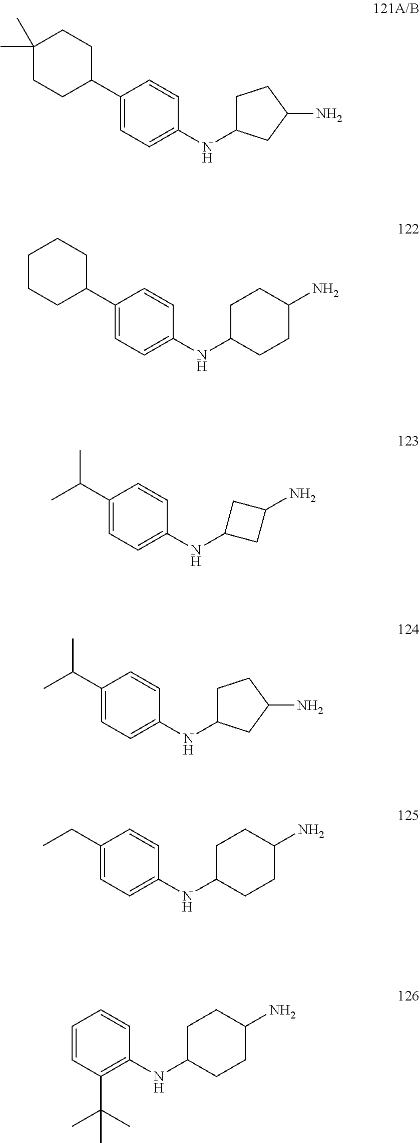 [SMILES: CC1(C)CCC(c2ccc(NC3CCC(N)C3)cc2)CC1 ; NC1CCC(Nc2ccc(C3CCCCC3)cc2)CC1 ; CC(C)c1ccc(NC2CC(N)C2)cc1 ; CC(C)c1ccc(NC2CCC(N)C2)cc1 ; CCc1ccc(NC2CCC(N)CC2)cc1 ; CC(C)(C)c1ccccc1NC1CCC(N)CC1] 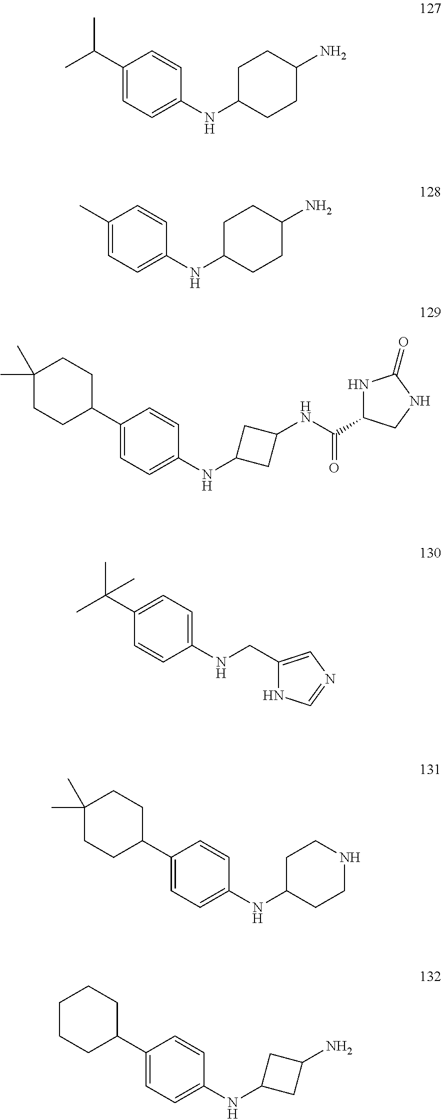 [SMILES: CC(C)c1ccc(NC2CCC(N)CC2)cc1 ; Cc1ccc(NC2CCC(N)CC2)cc1 ; CC1(C)CCC(c2ccc(NC3CC(NC(=O)[C@H]4CNC(=O)N4)C3)cc2)CC1 ; CC(C)(C)c1ccc(NCc2cnc[nH]2)cc1 ; CC1(C)CCC(c2ccc(NC3CCNCC3)cc2)CC1 ; NC1CC(Nc2ccc(C3CCCCC3)cc2)C1] 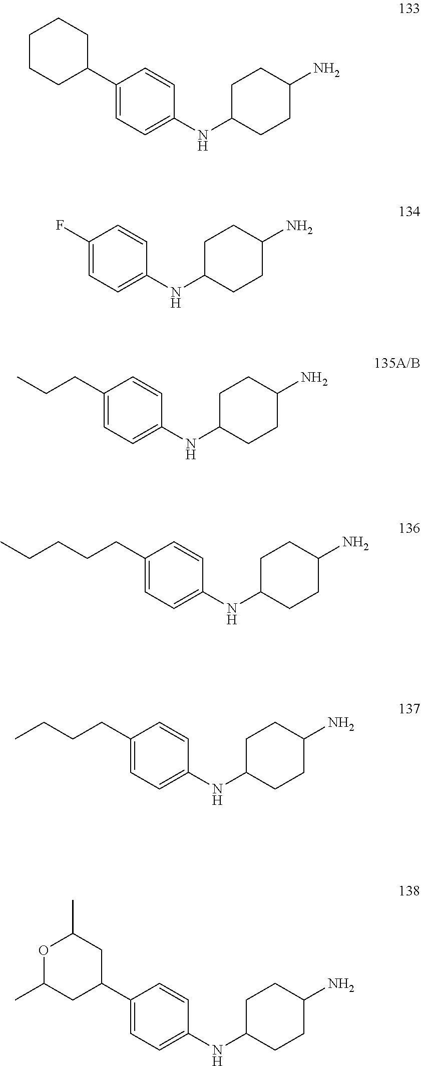 [SMILES: NC1CCC(Nc2ccc(C3CCCCC3)cc2)CC1 ; NC1CCC(Nc2ccc(F)cc2)CC1 ; CCCc1ccc(NC2CCC(N)CC2)cc1 ; CCCCCc1ccc(NC2CCC(N)CC2)cc1 ; CCCCc1ccc(NC2CCC(N)CC2)cc1 ; CC1CC(c2ccc(NC3CCC(N)CC3)cc2)CC(C)O1] 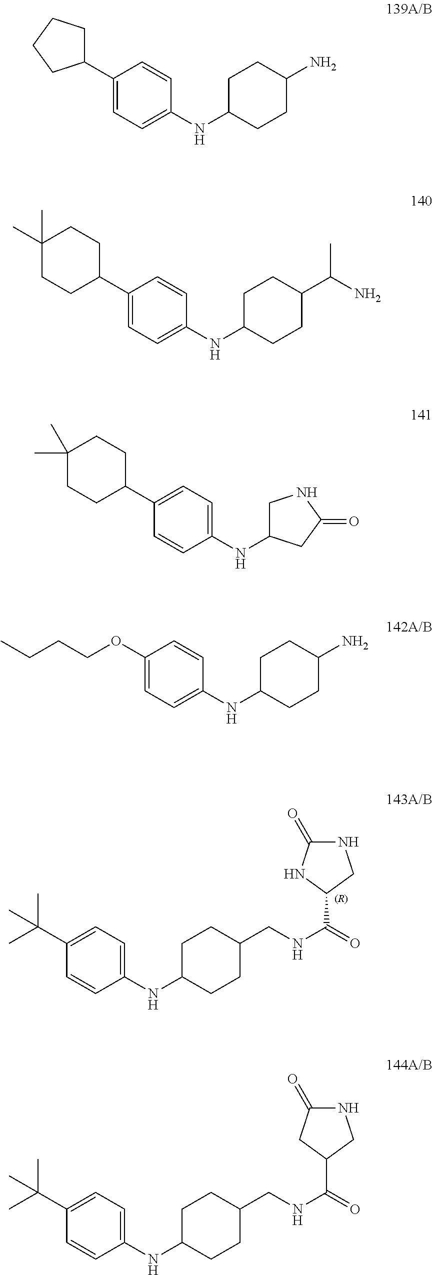 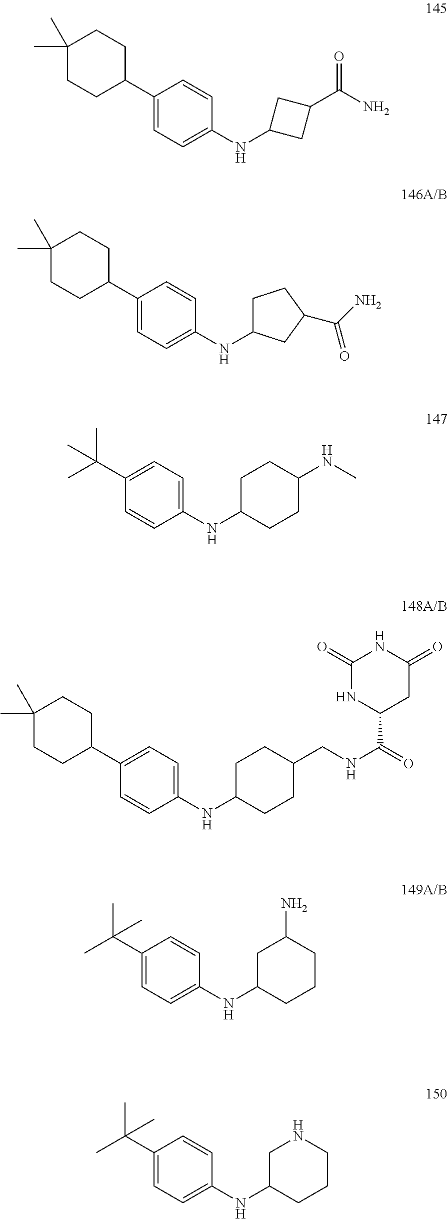 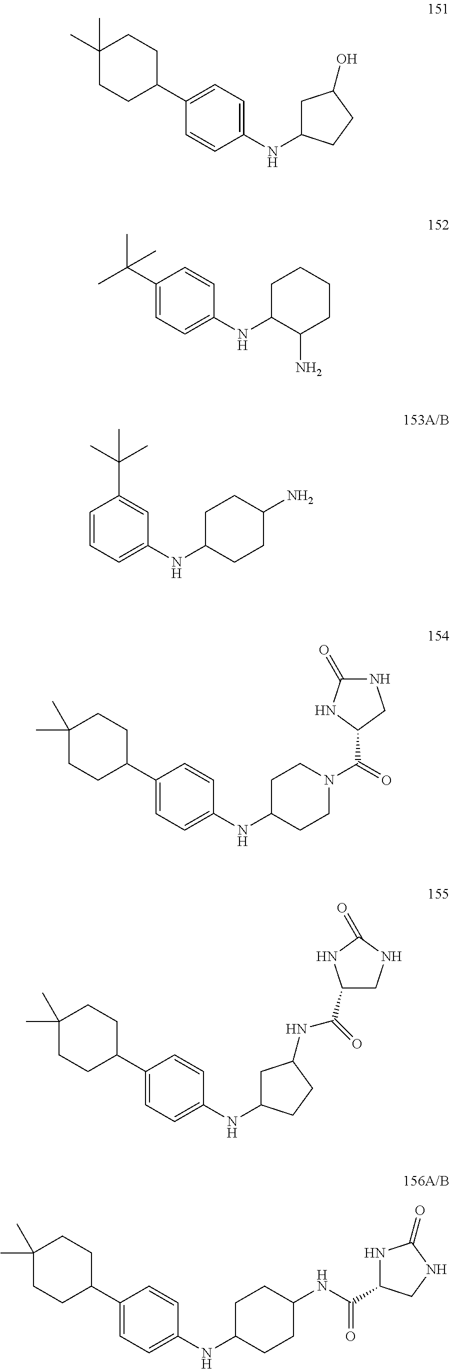 [SMILES: CC1(C)CCC(c2ccc(NC3CCC(O)C3)cc2)CC1 ; CC(C)(C)c1ccc(NC2CCCCC2N)cc1 ; CC(C)(C)c1cccc(NC2CCC(N)CC2)c1 ; CC1(C)CCC(c2ccc(NC3CCN(C(=O)[C@H]4CNC(=O)N4)CC3)cc2)CC1 ; CC1(C)CCC(c2ccc(NC3CCC(NC(=O)[C@H]4CNC(=O)N4)C3)cc2)CC1 ; CC1(C)CCC(c2ccc(NC3CCC(NC(=O)[C@H]4CNC(=O)N4)CC3)cc2)CC1] 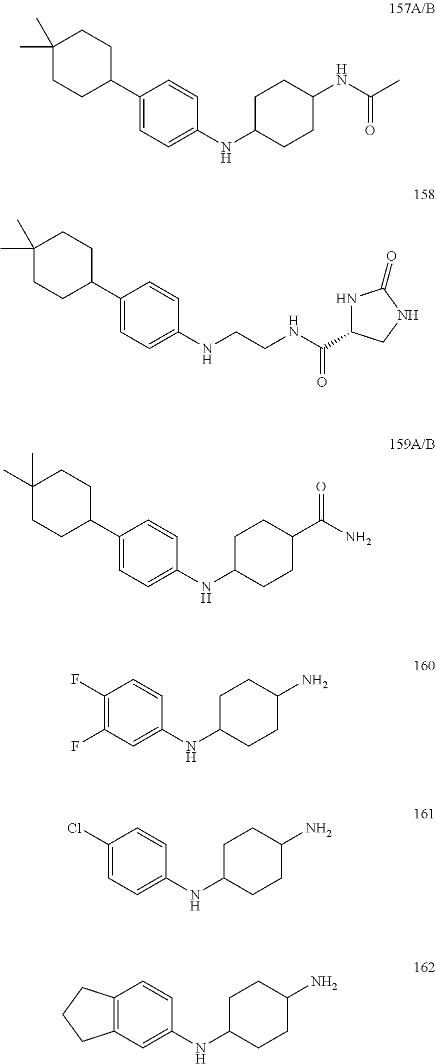 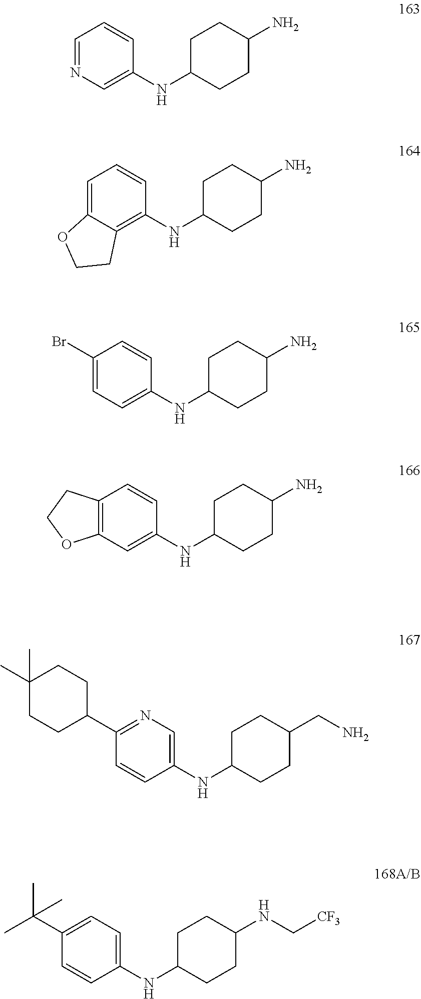 [SMILES: NC1CCC(Nc2cccnc2)CC1 ; NC1CCC(Nc2cccc3c2CCO3)CC1 ; NC1CCC(Nc2ccc(Br)cc2)CC1 ; NC1CCC(Nc2ccc3c(c2)OCC3)CC1 ; CC1(C)CCC(c2ccc(NC3CCC(CN)CC3)cn2)CC1 ; CC(C)(C)c1ccc(NC2CCC(NCC(F)(F)F)CC2)cc1] 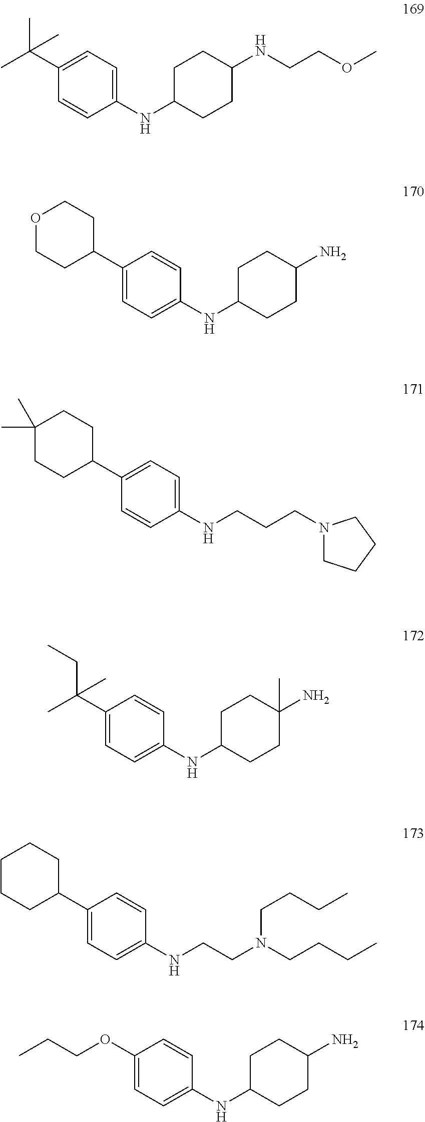 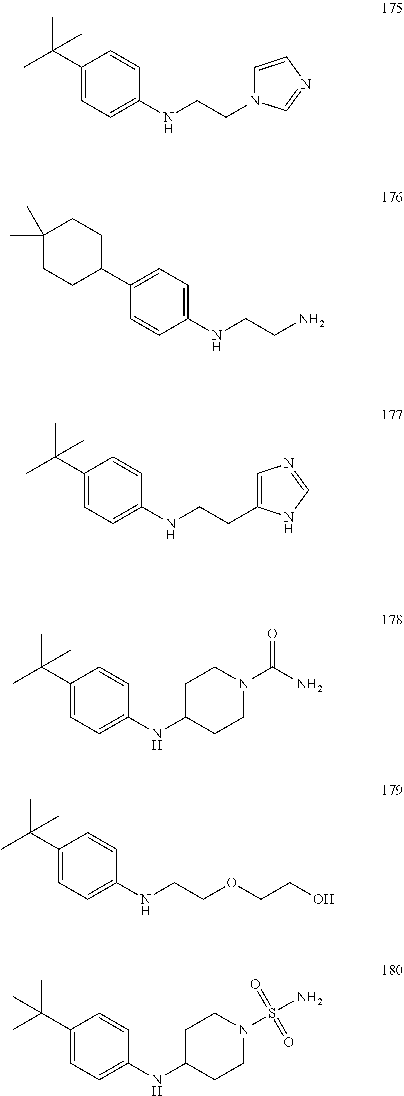 [SMILES: CC(C)(C)c1ccc(NCCn2ccnc2)cc1 ; CC1(C)CCC(c2ccc(NCCN)cc2)CC1 ; CC(C)(C)c1ccc(NCCc2cnc[nH]2)cc1 ; CC(C)(C)c1ccc(NC2CCN(C(N)=O)CC2)cc1 ; CC(C)(C)c1ccc(NCCOCCO)cc1 ; CC(C)(C)c1ccc(NC2CCN(S(N)(=O)=O)CC2)cc1] 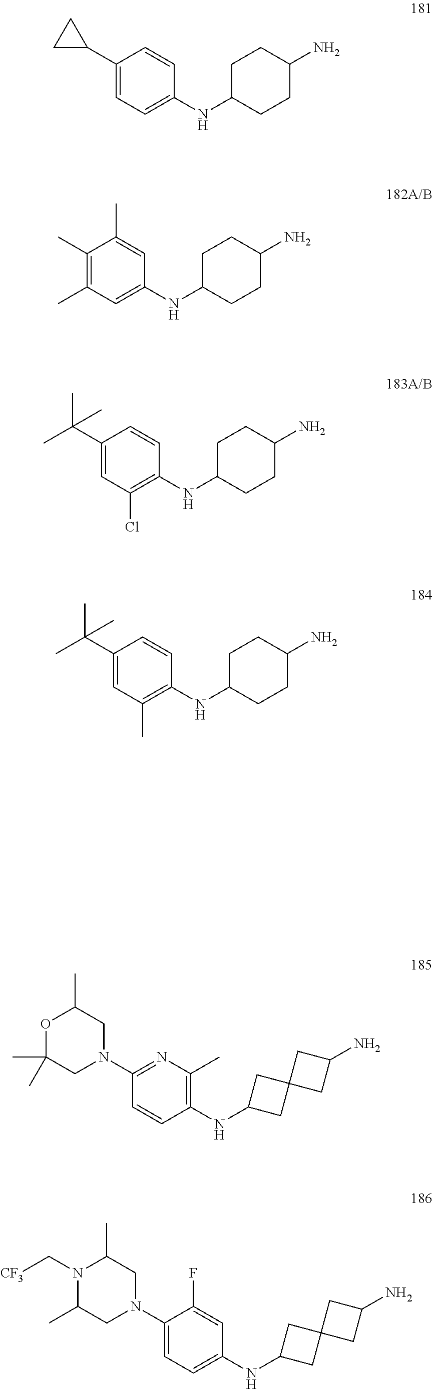 [SMILES: NC1CCC(Nc2ccc(C3CC3)cc2)CC1 ; Cc1cc(NC2CCC(N)CC2)cc(C)c1C ; CC(C)(C)c1ccc(NC2CCC(N)CC2)c(Cl)c1 ; Cc1cc(C(C)(C)C)ccc1NC1CCC(N)CC1 ; Cc1nc(N2CC(C)OC(C)(C)C2)ccc1NC1CC2(CC(N)C2)C1 ; CC1CN(c2ccc(NC3CC4(CC(N)C4)C3)cc2F)CC(C)N1CC(F)(F)F] 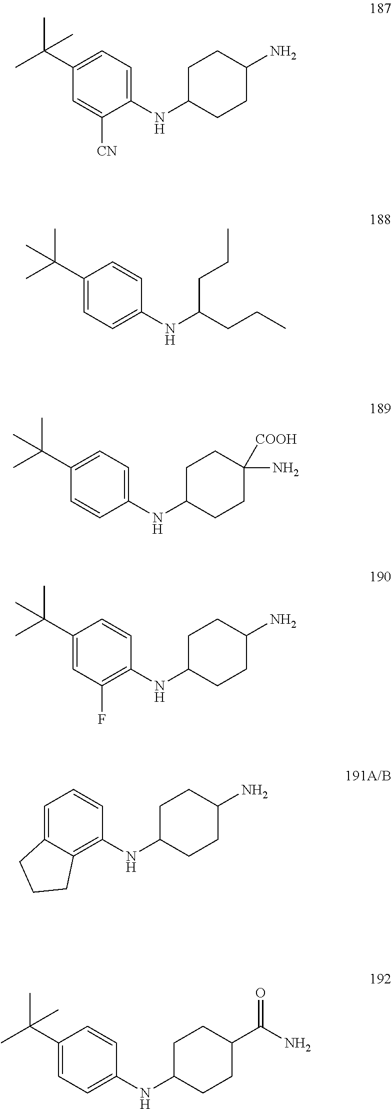 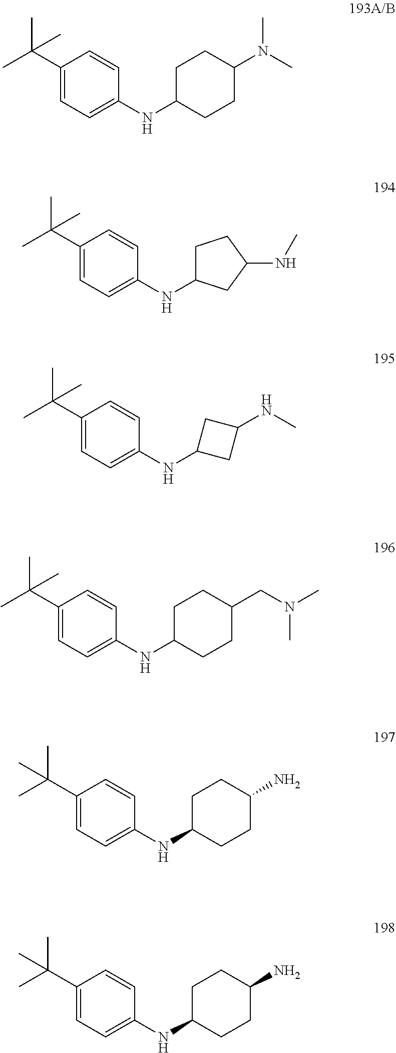 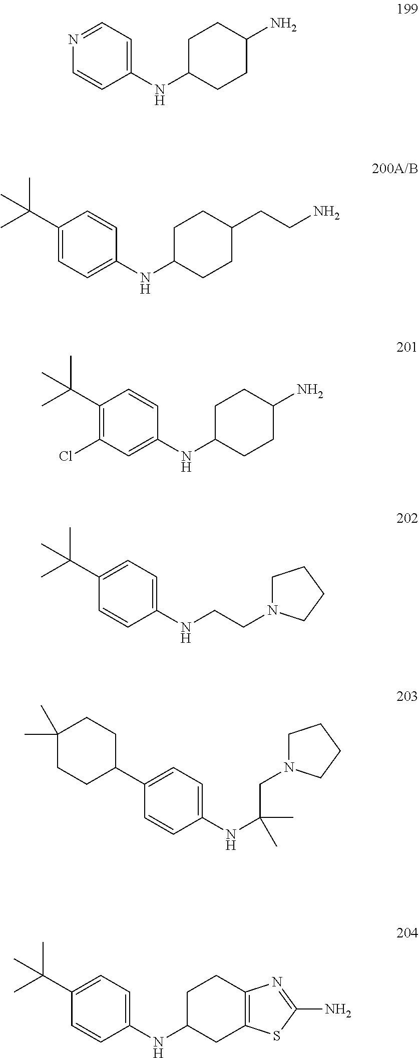 [SMILES: NC1CCC(Nc2ccncc2)CC1 ; CC(C)(C)c1ccc(NC2CCC(CCN)CC2)cc1 ; CC(C)(C)c1ccc(NC2CCC(N)CC2)cc1Cl ; CC(C)(C)c1ccc(NCCN2CCCC2)cc1 ; CC1(C)CCC(c2ccc(NC(C)(C)CN3CCCC3)cc2)CC1 ; CC(C)(C)c1ccc(NC2CCc3nc(N)sc3C2)cc1] 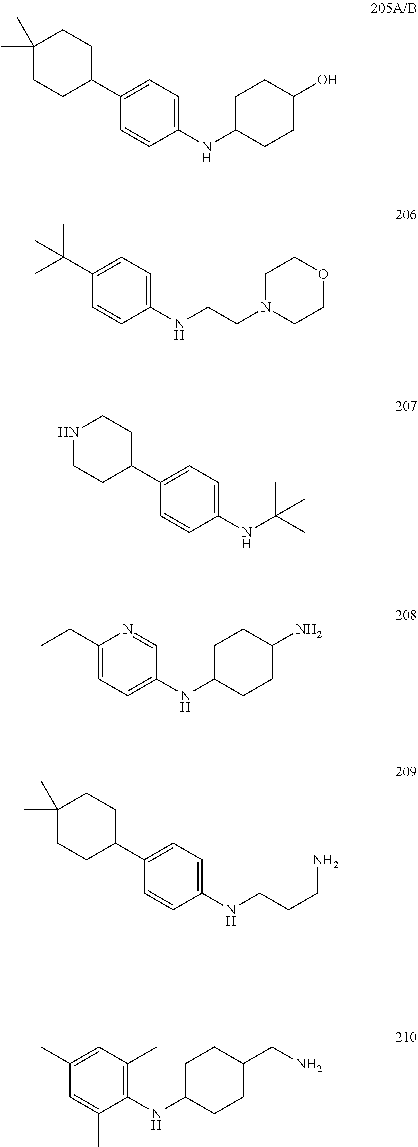 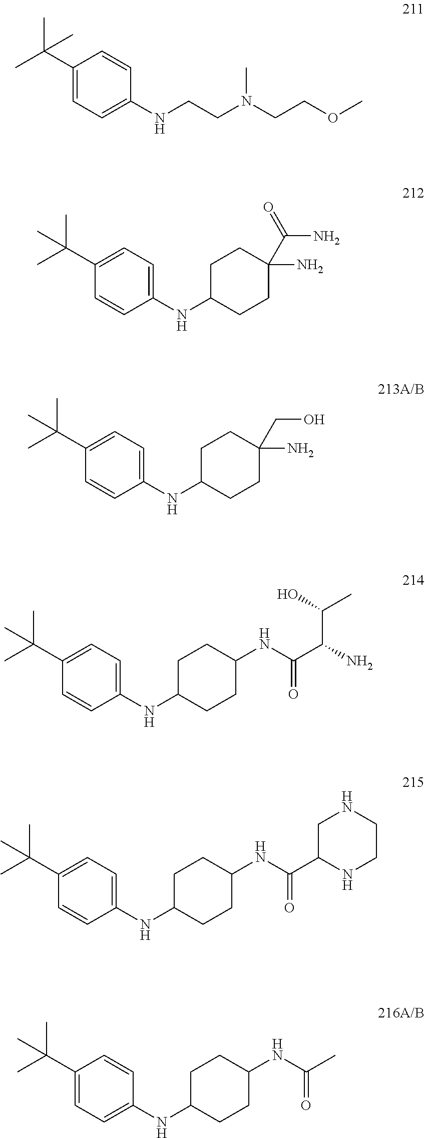 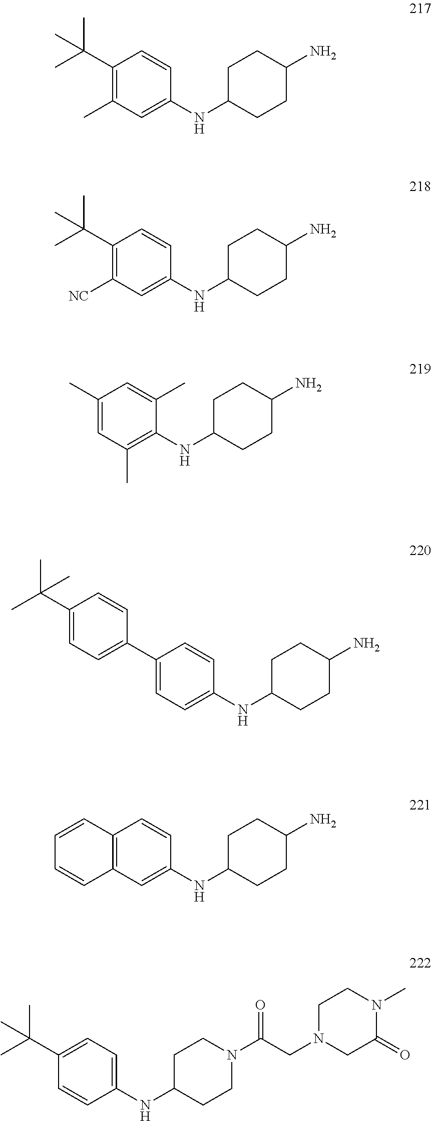 [SMILES: Cc1cc(NC2CCC(N)CC2)ccc1C(C)(C)C ; CC(C)(C)c1ccc(NC2CCC(N)CC2)cc1C#N ; Cc1cc(C)c(NC2CCC(N)CC2)c(C)c1 ; CC(C)(C)c1ccc(-c2ccc(NC3CCC(N)CC3)cc2)cc1 ; NC1CCC(Nc2ccc3ccccc3c2)CC1 ; CN1CCN(CC(=O)N2CCC(Nc3ccc(C(C)(C)C)cc3)CC2)CC1=O] 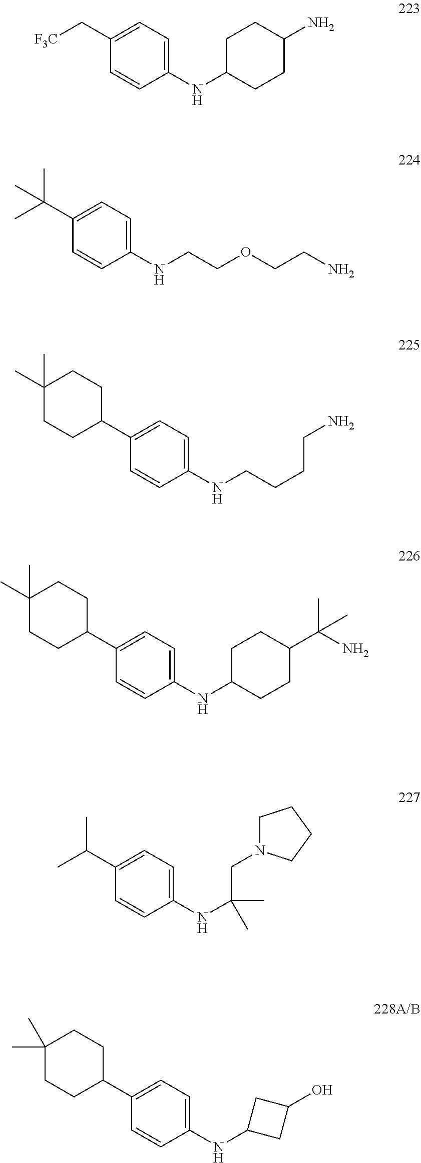 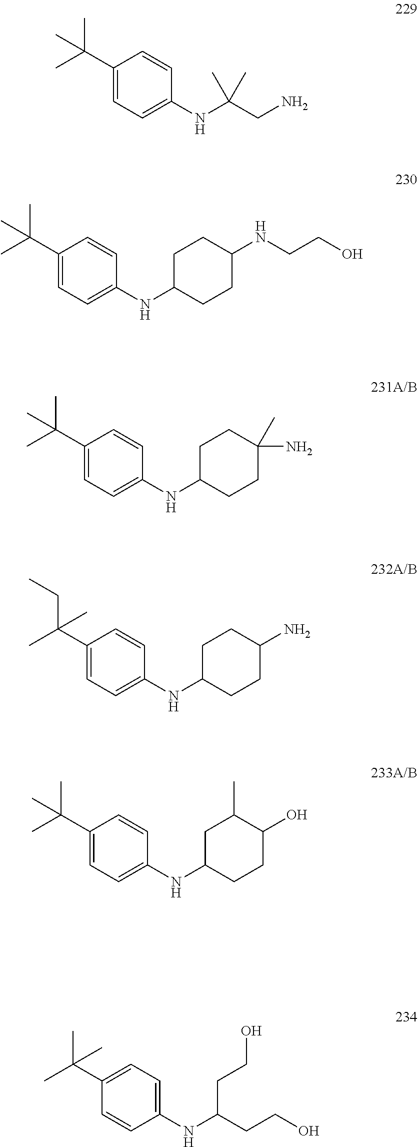 [SMILES: CC(C)(CN)Nc1ccc(C(C)(C)C)cc1 ; CC(C)(C)c1ccc(NC2CCC(NCCO)CC2)cc1 ; CC1(N)CCC(Nc2ccc(C(C)(C)C)cc2)CC1 ; CCC(C)(C)c1ccc(NC2CCC(N)CC2)cc1 ; CC1CC(Nc2ccc(C(C)(C)C)cc2)CCC1O ; CC(C)(C)c1ccc(NC(CCO)CCO)cc1] 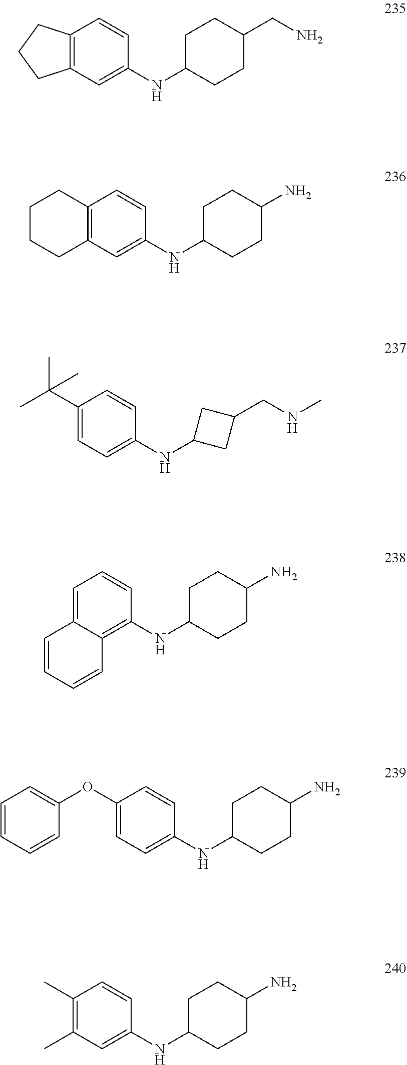 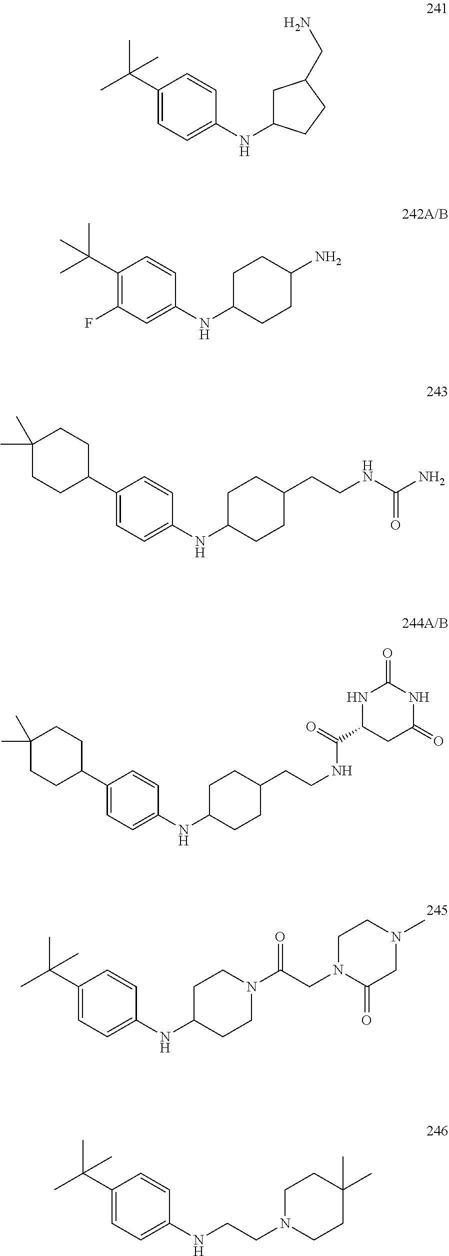 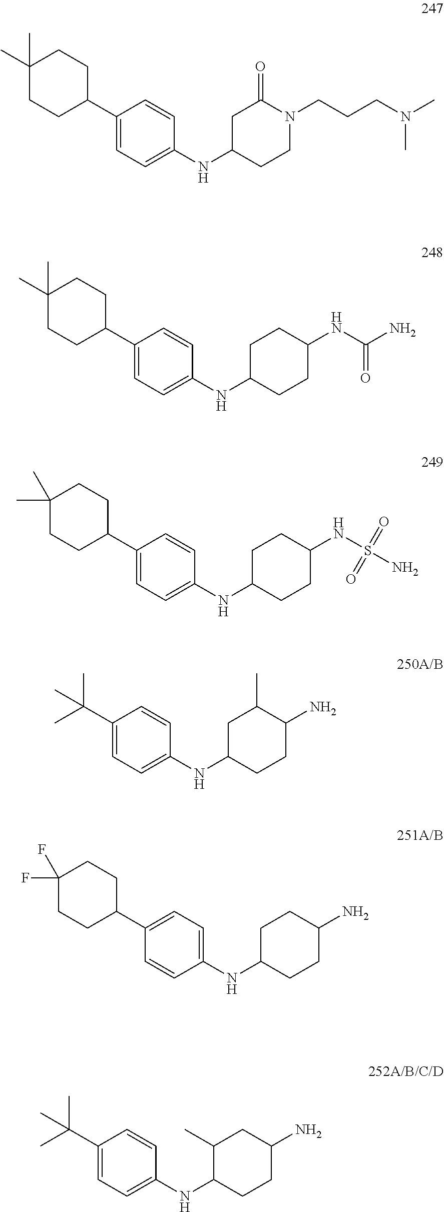 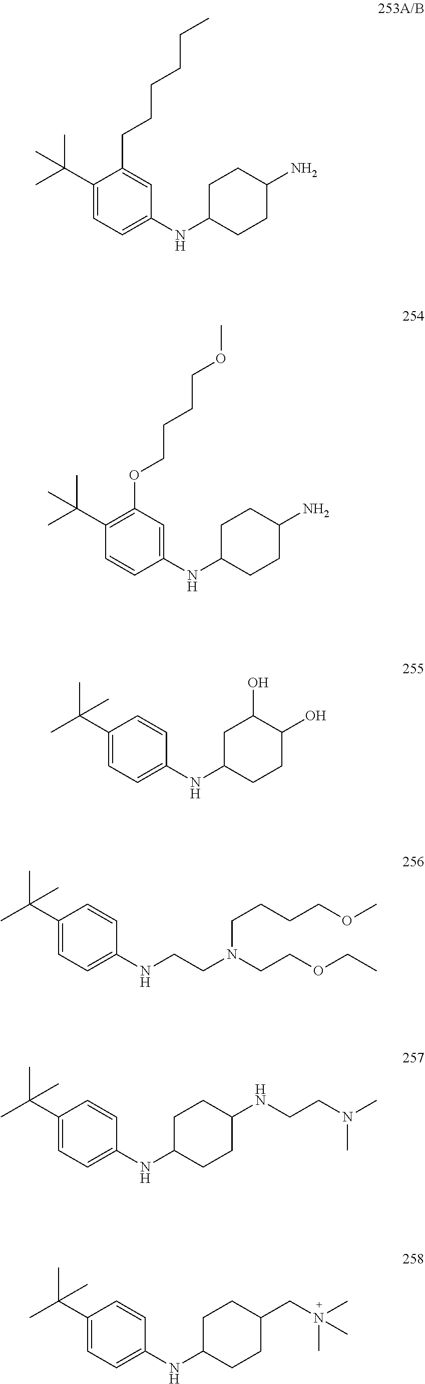 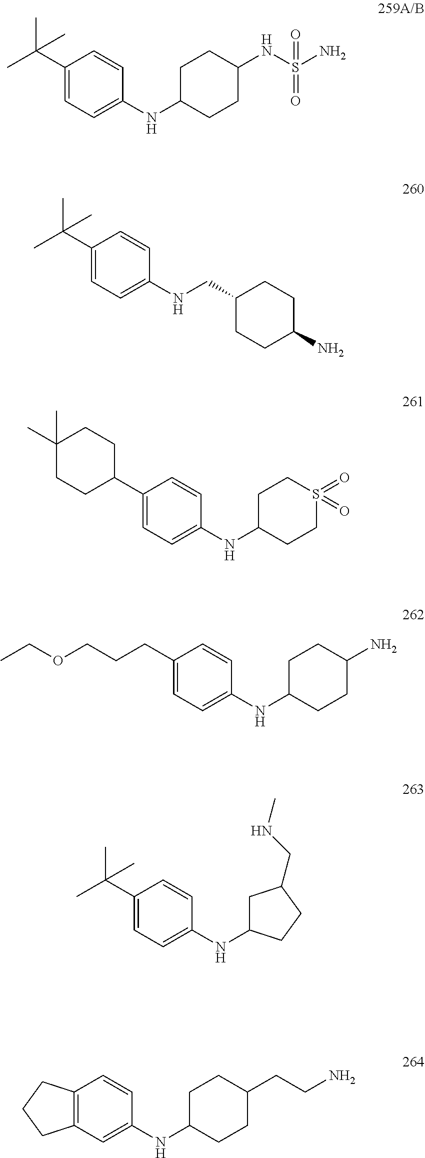 [SMILES: CC(C)(C)c1ccc(NC2CCC(NS(N)(=O)=O)CC2)cc1 ; CC(C)(C)c1ccc(NC[C@H]2CC[C@H](N)CC2)cc1 ; CC1(C)CCC(c2ccc(NC3CCS(=O)(=O)CC3)cc2)CC1 ; CCOCCCc1ccc(NC2CCC(N)CC2)cc1 ; CNCC1CCC(Nc2ccc(C(C)(C)C)cc2)C1 ; NCCC1CCC(Nc2ccc3c(c2)CCC3)CC1] 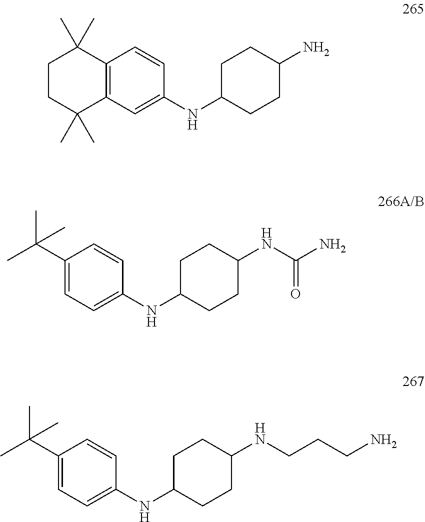 [SMILES: CC1(C)CCC(C)(C)c2cc(NC3CCC(N)CC3)ccc21 ; CC(C)(C)c1ccc(NC2CCC(NC(N)=O)CC2)cc1 ; CC(C)(C)c1ccc(NC2CCC(NCCCN)CC2)cc1] 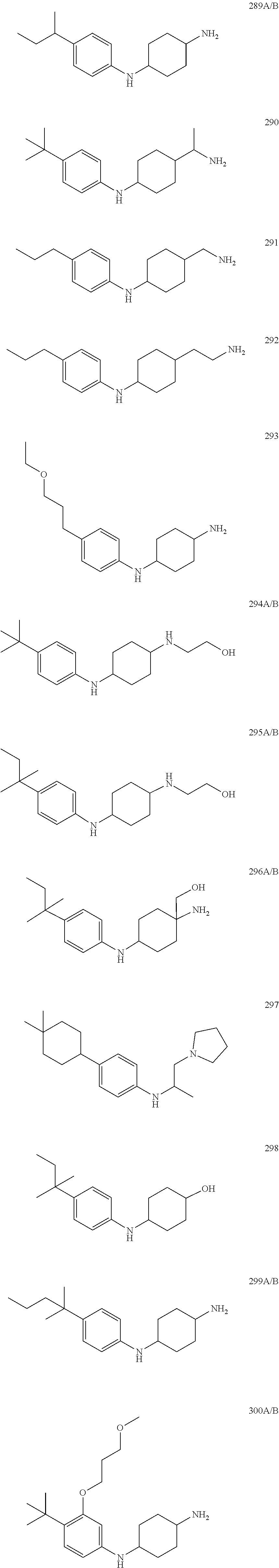 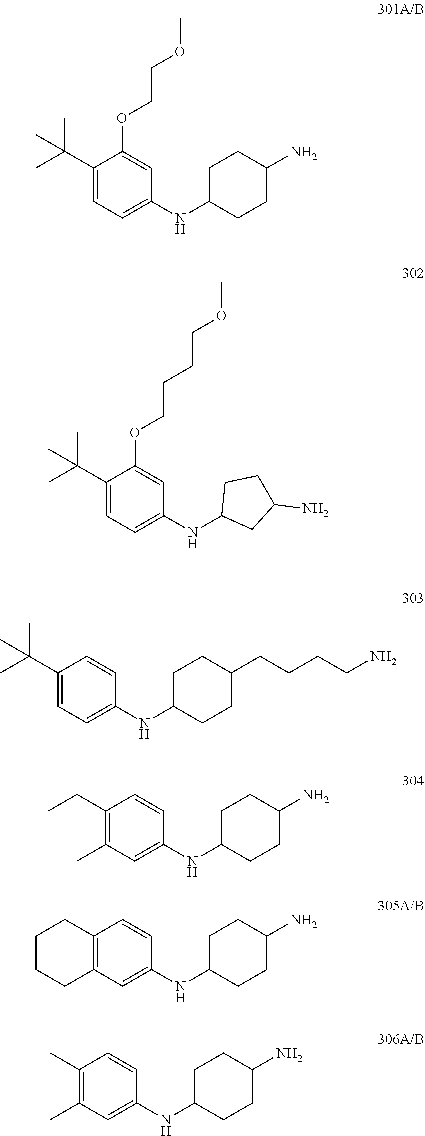 [SMILES: COCCOc1cc(NC2CCC(N)CC2)ccc1C(C)(C)C ; COCCCCOc1cc(NC2CCC(N)C2)ccc1C(C)(C)C ; CC(C)(C)c1ccc(NC2CCC(CCCCN)CC2)cc1 ; CCc1ccc(NC2CCC(N)CC2)cc1C ; NC1CCC(Nc2ccc3c(c2)CCCC3)CC1 ; Cc1ccc(NC2CCC(N)CC2)cc1C] 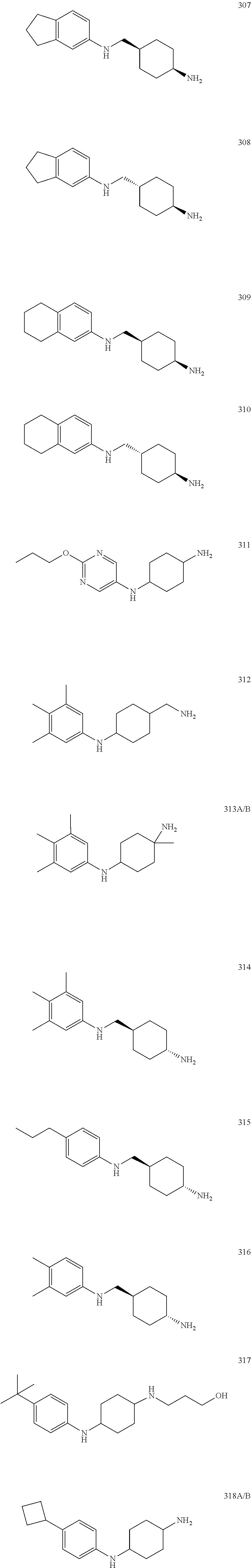 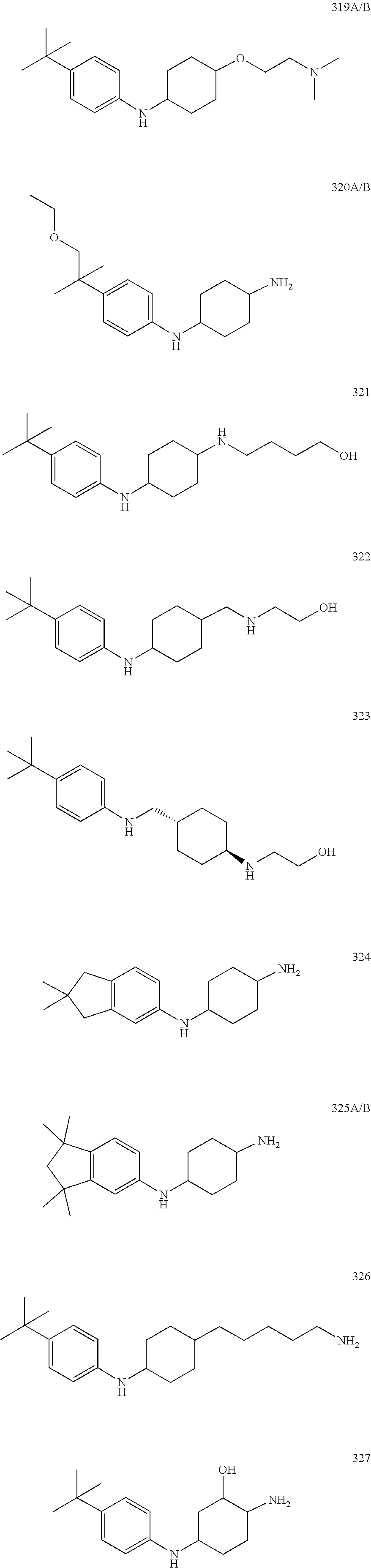 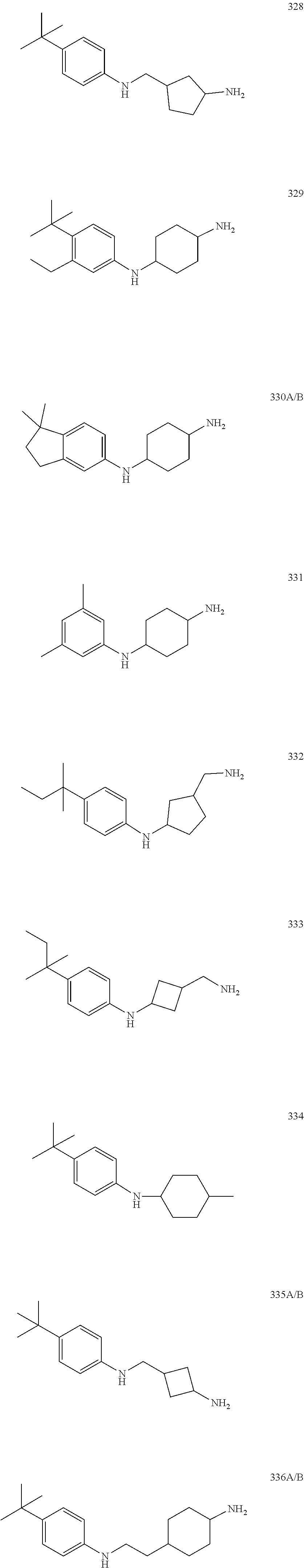 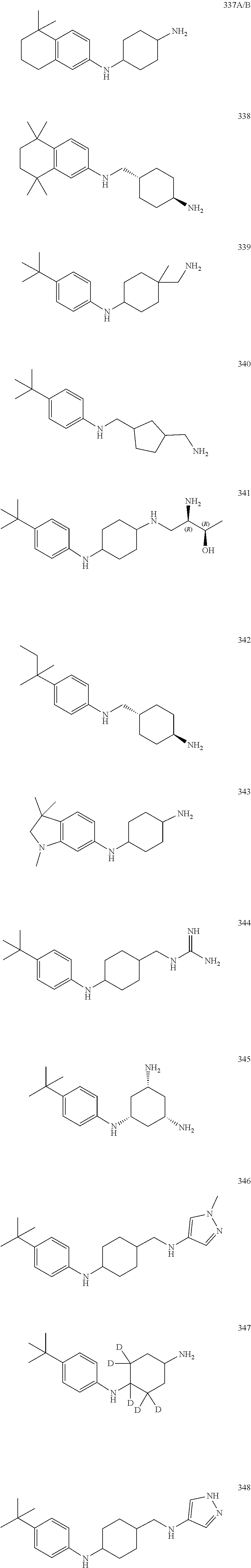 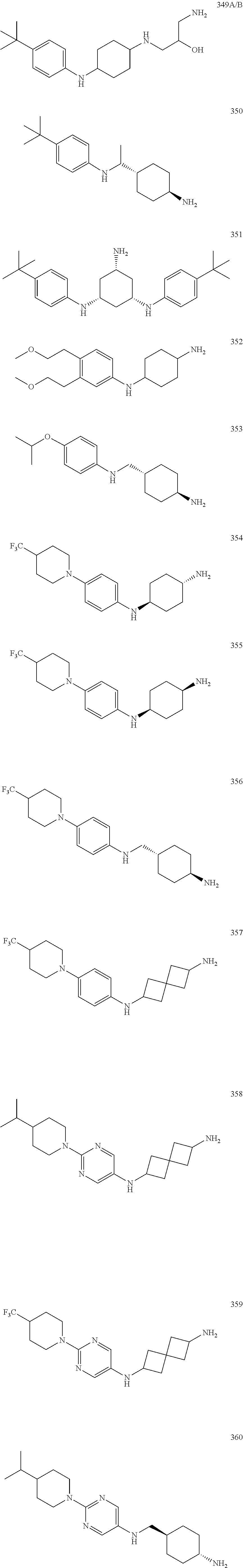 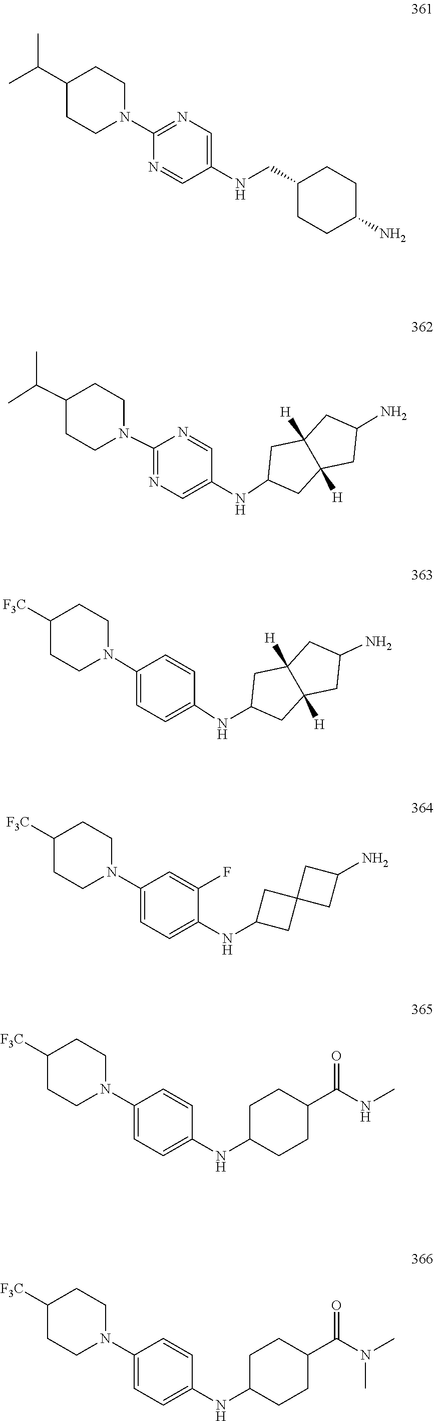 [SMILES: CC(C)C1CCN(c2ncc(NC[C@H]3CC[C@@H](N)CC3)cn2)CC1 ; CC(C)C1CCN(c2ncc(NC3C[C@H]4CC(N)C[C@H]4C3)cn2)CC1 ; NC1C[C@@H]2CC(Nc3ccc(N4CCC(C(F)(F)F)CC4)cc3)C[C@@H]2C1 ; NC1CC2(C1)CC(Nc1ccc(N3CCC(C(F)(F)F)CC3)cc1F)C2 ; CNC(=O)C1CCC(Nc2ccc(N3CCC(C(F)(F)F)CC3)cc2)CC1 ; CN(C)C(=O)C1CCC(Nc2ccc(N3CCC(C(F)(F)F)CC3)cc2)CC1] 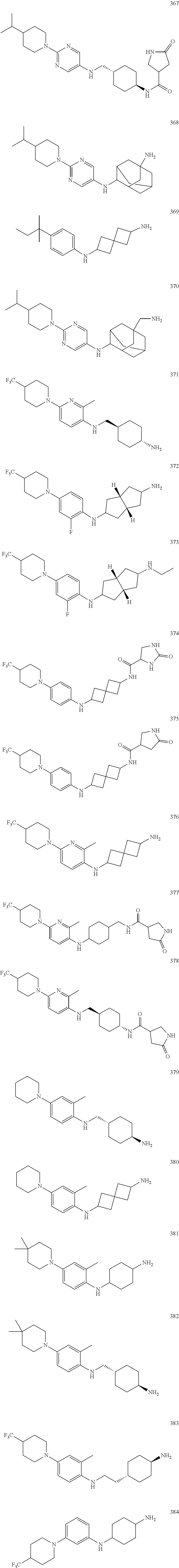 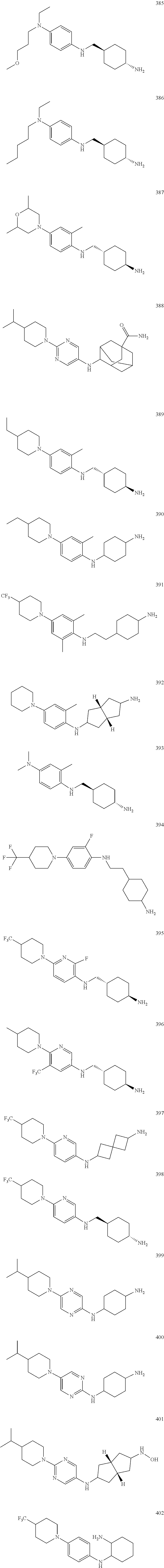 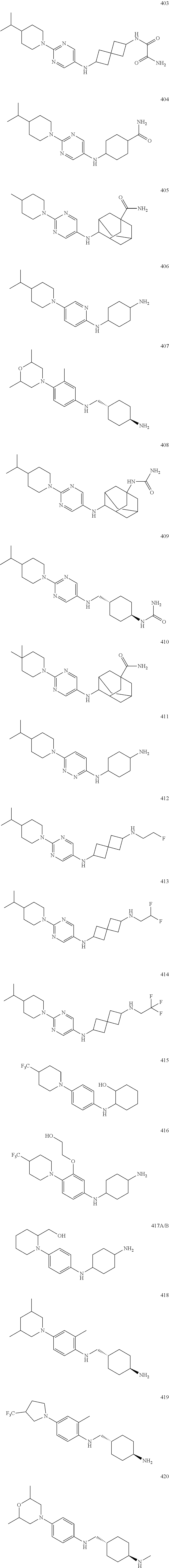 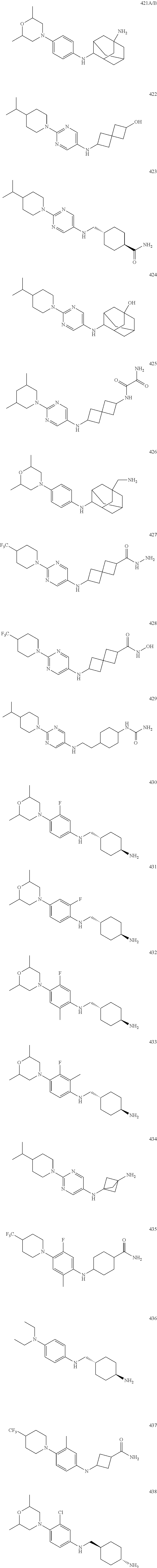 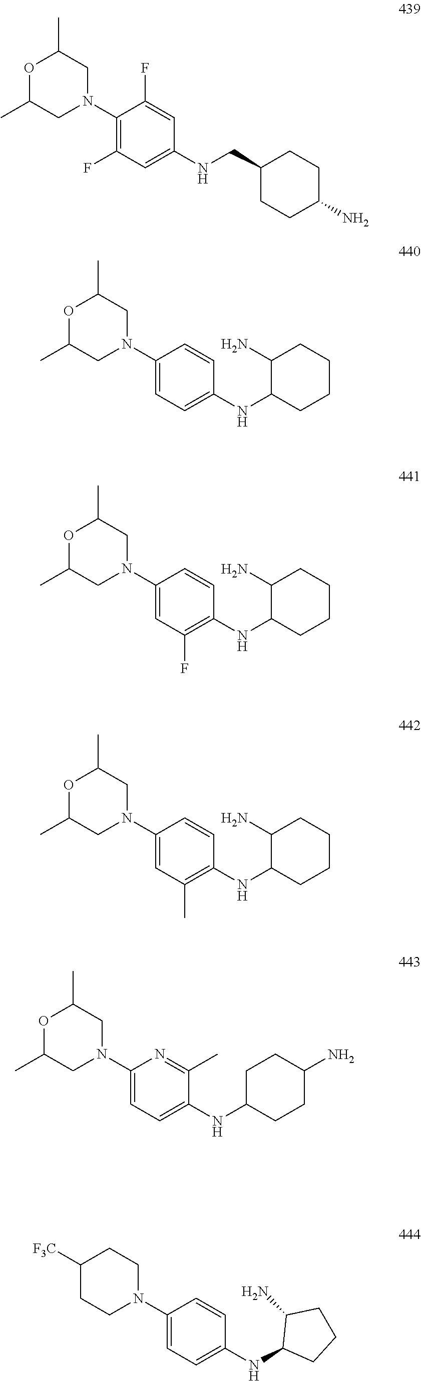 [SMILES: CC1CN(c2c(F)cc(NC[C@H]3CC[C@H](N)CC3)cc2F)CC(C)O1 ; CC1CN(c2ccc(NC3CCCCC3N)cc2)CC(C)O1 ; CC1CN(c2ccc(NC3CCCCC3N)c(F)c2)CC(C)O1 ; Cc1cc(N2CC(C)OC(C)C2)ccc1NC1CCCCC1N ; Cc1nc(N2CC(C)OC(C)C2)ccc1NC1CCC(N)CC1 ; N[C@@H]1CCC[C@H]1Nc1ccc(N2CCC(C(F)(F)F)CC2)cc1] 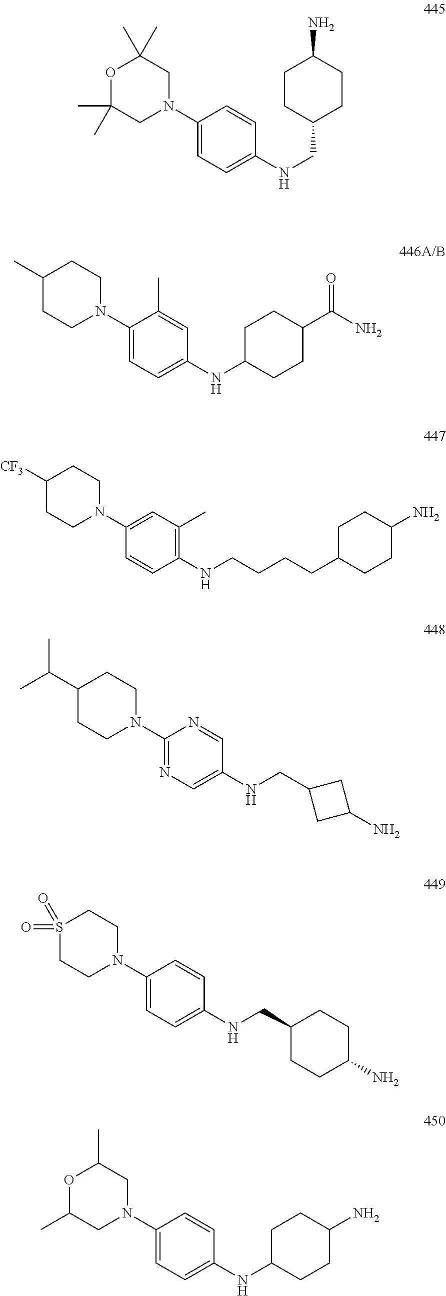 [SMILES: CC1(C)CN(c2ccc(NC[C@H]3CC[C@H](N)CC3)cc2)CC(C)(C)O1 ; Cc1cc(NC2CCC(C(N)=O)CC2)ccc1N1CCC(C)CC1 ; Cc1cc(N2CCC(C(F)(F)F)CC2)ccc1NCCCCC1CCC(N)CC1 ; CC(C)C1CCN(c2ncc(NCC3CC(N)C3)cn2)CC1 ; N[C@H]1CC[C@H](CNc2ccc(N3CCS(=O)(=O)CC3)cc2)CC1 ; CC1CN(c2ccc(NC3CCC(N)CC3)cc2)CC(C)O1] 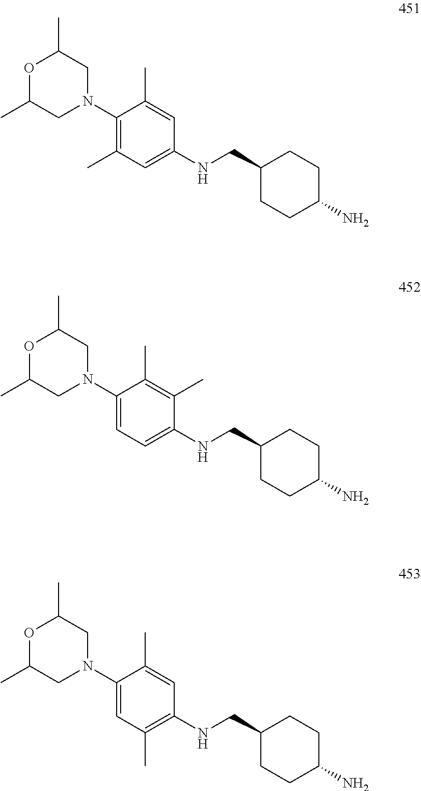 [SMILES: Cc1cc(NC[C@H]2CC[C@H](N)CC2)cc(C)c1N1CC(C)OC(C)C1 ; Cc1c(NC[C@H]2CC[C@H](N)CC2)ccc(N2CC(C)OC(C)C2)c1C ; Cc1cc(N2CC(C)OC(C)C2)c(C)cc1NC[C@H]1CC[C@H](N)CC1] 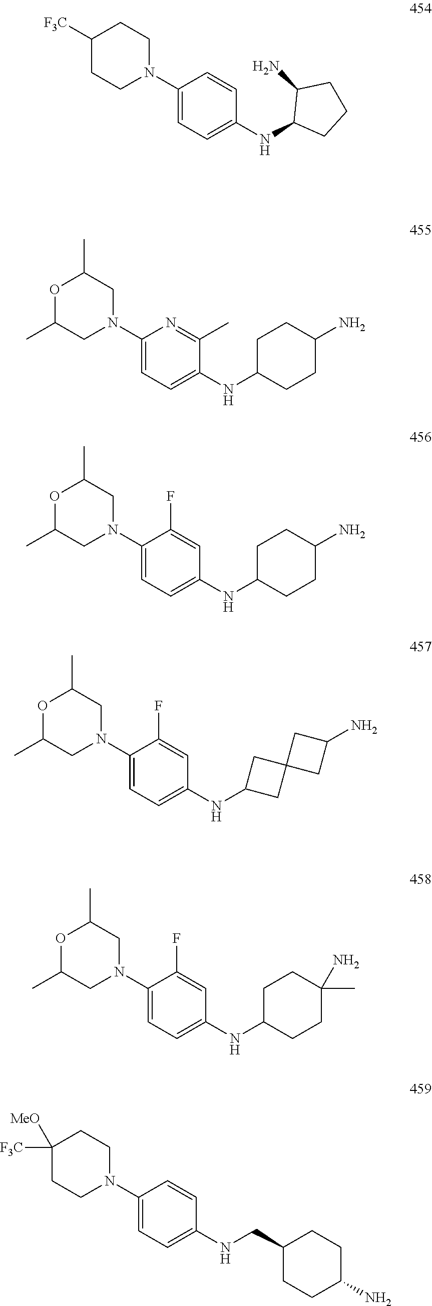 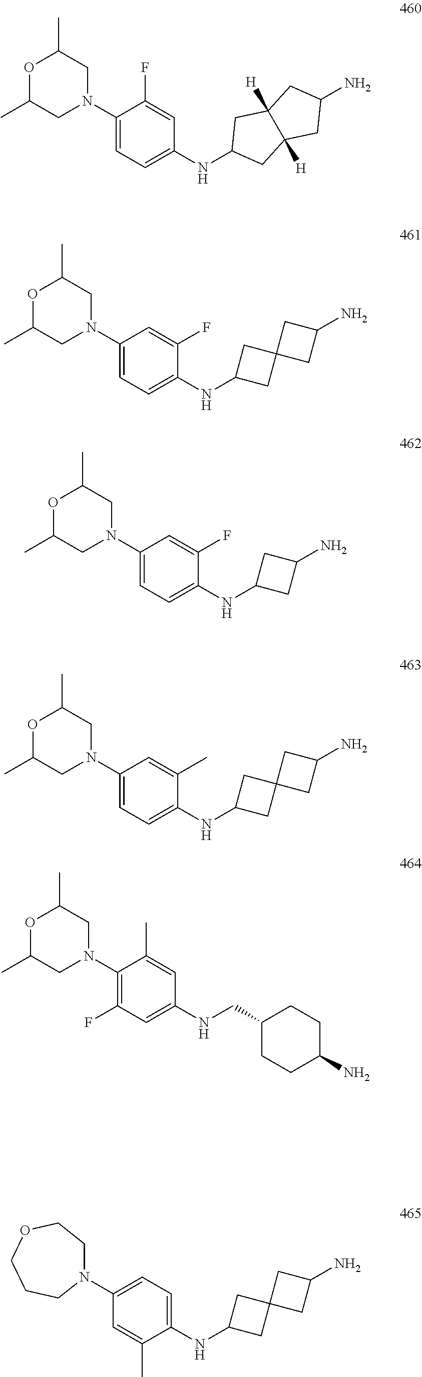 [SMILES: CC1CN(c2ccc(NC3C[C@H]4CC(N)C[C@H]4C3)cc2F)CC(C)O1 ; CC1CN(c2ccc(NC3CC4(CC(N)C4)C3)c(F)c2)CC(C)O1 ; CC1CN(c2ccc(NC3CC(N)C3)c(F)c2)CC(C)O1 ; Cc1cc(N2CC(C)OC(C)C2)ccc1NC1CC2(CC(N)C2)C1 ; Cc1cc(NC[C@H]2CC[C@H](N)CC2)cc(F)c1N1CC(C)OC(C)C1 ; Cc1cc(N2CCCOCC2)ccc1NC1CC2(CC(N)C2)C1] 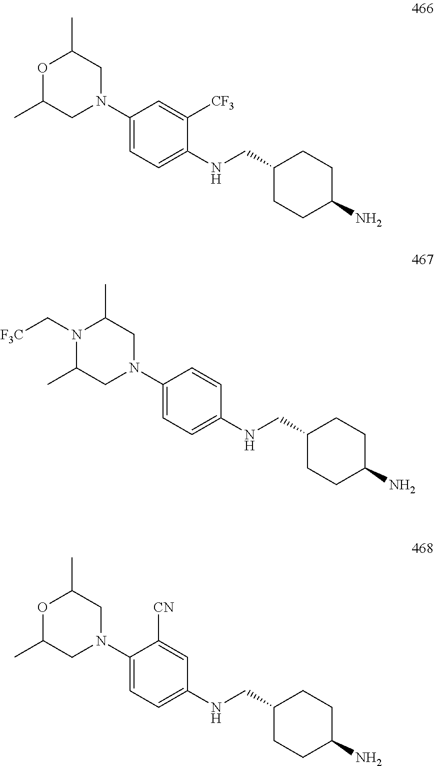 [SMILES: CC1CN(c2ccc(NC[C@H]3CC[C@H](N)CC3)c(C(F)(F)F)c2)CC(C)O1 ; CC1CN(c2ccc(NC[C@H]3CC[C@H](N)CC3)cc2)CC(C)N1CC(F)(F)F ; CC1CN(c2ccc(NC[C@H]3CC[C@H](N)CC3)cc2C#N)CC(C)O1] 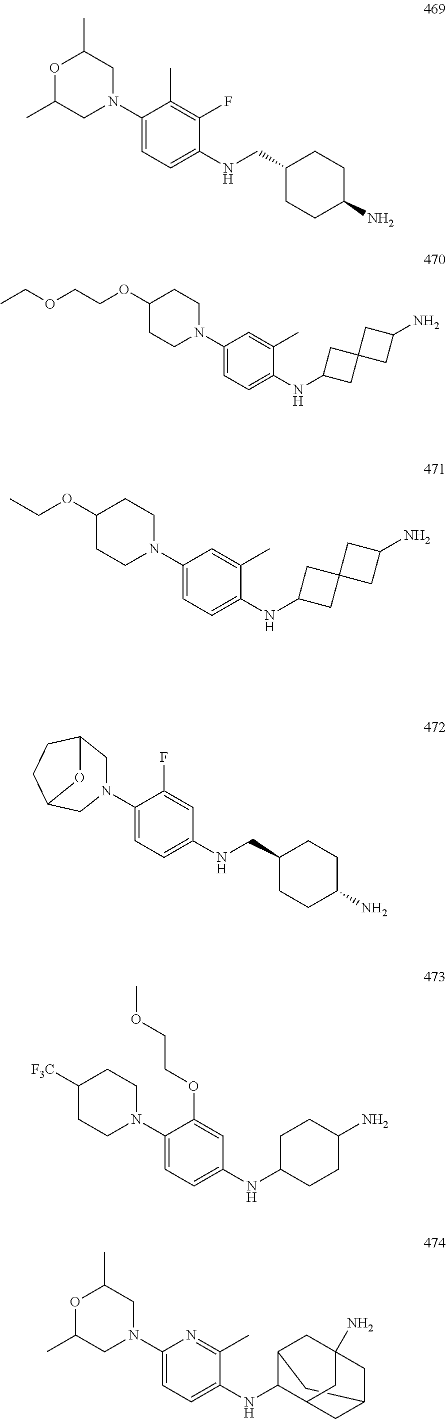 [SMILES: Cc1c(N2CC(C)OC(C)C2)ccc(NC[C@H]2CC[C@H](N)CC2)c1F ; CCOCCOC1CCN(c2ccc(NC3CC4(CC(N)C4)C3)c(C)c2)CC1 ; CCOC1CCN(c2ccc(NC3CC4(CC(N)C4)C3)c(C)c2)CC1 ; N[C@H]1CC[C@H](CNc2ccc(N3CC4CCC(C3)O4)c(F)c2)CC1 ; COCCOc1cc(NC2CCC(N)CC2)ccc1N1CCC(C(F)(F)F)CC1 ; Cc1nc(N2CC(C)OC(C)C2)ccc1NC1C2CC3CC1CC(N)(C3)C2] 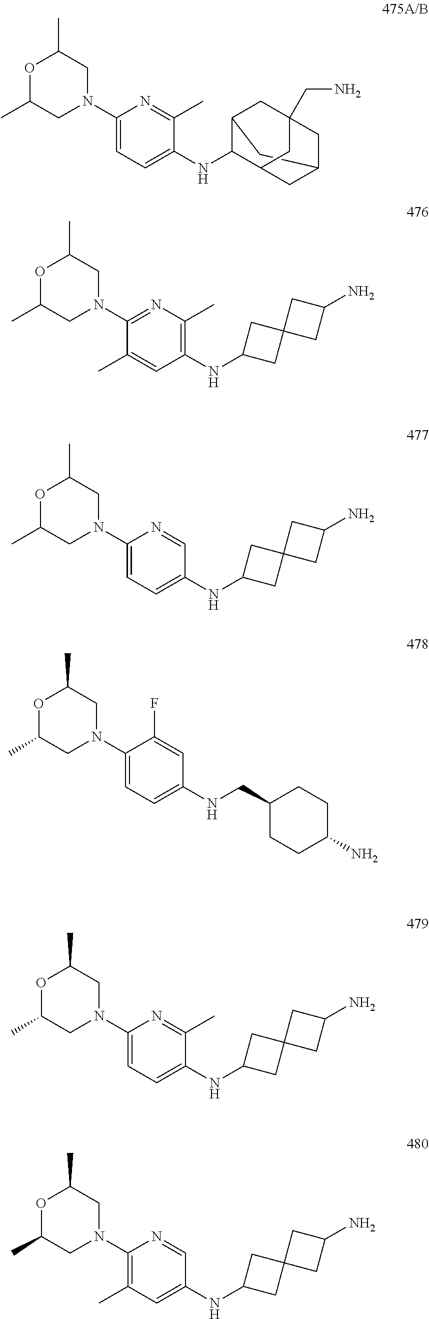 [SMILES: Cc1nc(N2CC(C)OC(C)C2)ccc1NC1C2CC3CC1CC(CN)(C3)C2 ; Cc1cc(NC2CC3(CC(N)C3)C2)c(C)nc1N1CC(C)OC(C)C1 ; CC1CN(c2ccc(NC3CC4(CC(N)C4)C3)cn2)CC(C)O1 ; C[C@H]1CN(c2ccc(NC[C@H]3CC[C@H](N)CC3)cc2F)C[C@H](C)O1 ; Cc1nc(N2C[C@H](C)O[C@@H](C)C2)ccc1NC1CC2(CC(N)C2)C1 ; Cc1cc(NC2CC3(CC(N)C3)C2)cnc1N1C[C@@H](C)O[C@@H](C)C1] 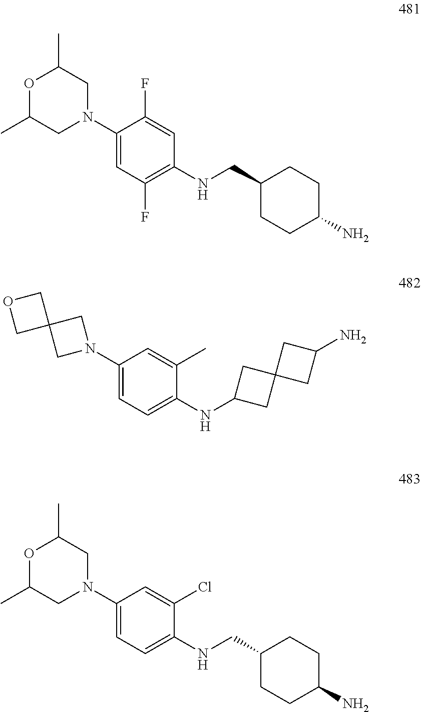 [SMILES: CC1CN(c2cc(F)c(NC[C@H]3CC[C@H](N)CC3)cc2F)CC(C)O1 ; Cc1cc(N2CC3(COC3)C2)ccc1NC1CC2(CC(N)C2)C1 ; CC1CN(c2ccc(NC[C@H]3CC[C@H](N)CC3)c(Cl)c2)CC(C)O1] 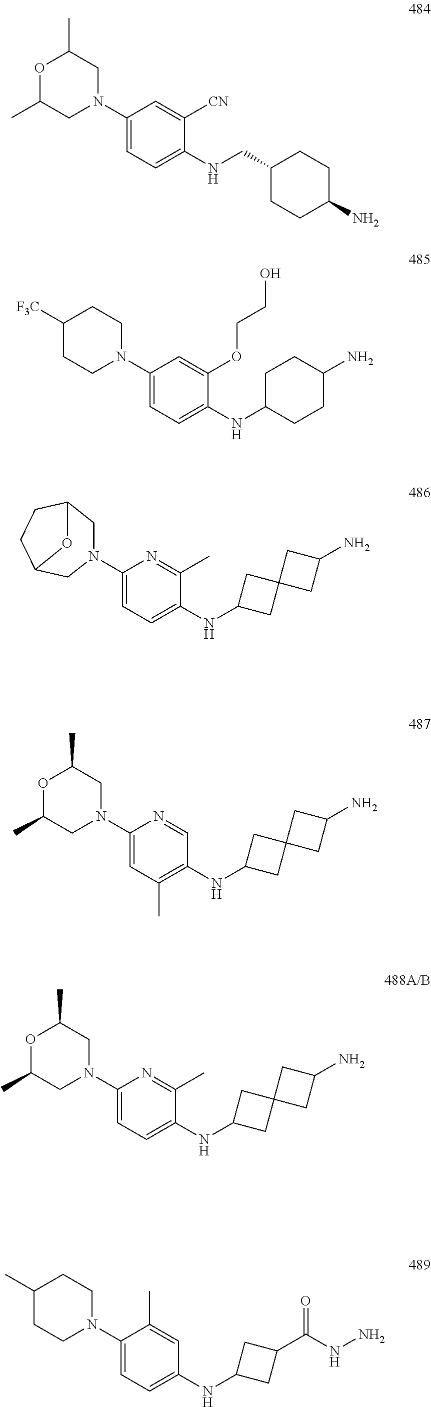 [SMILES: CC1CN(c2ccc(NC[C@H]3CC[C@H](N)CC3)c(C#N)c2)CC(C)O1 ; NC1CCC(Nc2ccc(N3CCC(C(F)(F)F)CC3)cc2OCCO)CC1 ; Cc1nc(N2CC3CCC(C2)O3)ccc1NC1CC2(CC(N)C2)C1 ; Cc1cc(N2C[C@@H](C)O[C@@H](C)C2)ncc1NC1CC2(CC(N)C2)C1 ; Cc1nc(N2C[C@@H](C)O[C@@H](C)C2)ccc1NC1CC2(CC(N)C2)C1 ; Cc1cc(NC2CC(C(=O)NN)C2)ccc1N1CCC(C)CC1] 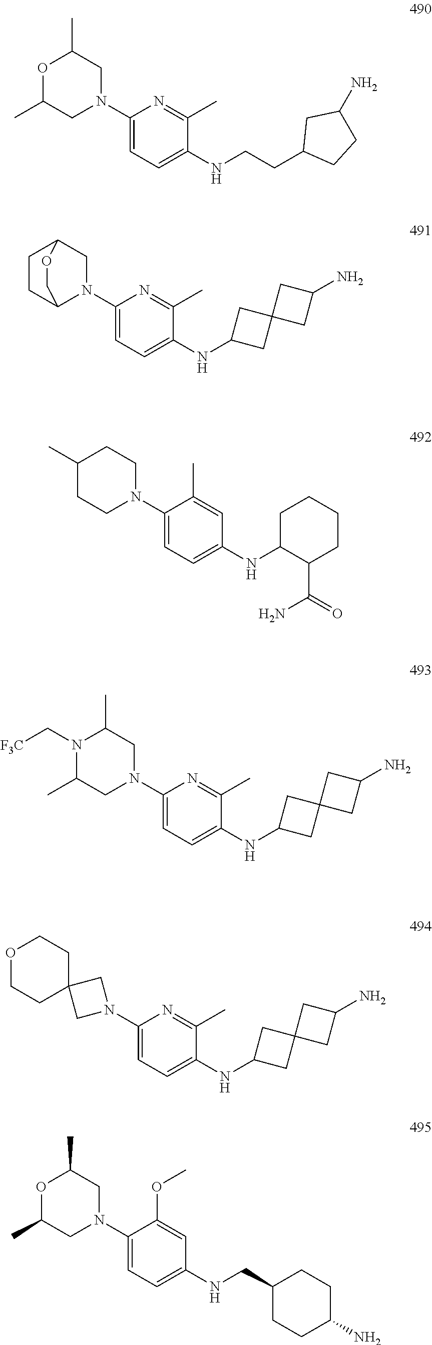 [SMILES: Cc1nc(N2CC(C)OC(C)C2)ccc1NCCC1CCC(N)C1 ; Cc1nc(N2CC3CCC2CO3)ccc1NC1CC2(CC(N)C2)C1 ; Cc1cc(NC2CCCCC2C(N)=O)ccc1N1CCC(C)CC1 ; Cc1nc(N2CC(C)N(CC(F)(F)F)C(C)C2)ccc1NC1CC2(CC(N)C2)C1 ; Cc1nc(N2CC3(CCOCC3)C2)ccc1NC1CC2(CC(N)C2)C1 ; COc1cc(NC[C@H]2CC[C@H](N)CC2)ccc1N1C[C@@H](C)O[C@@H](C)C1] 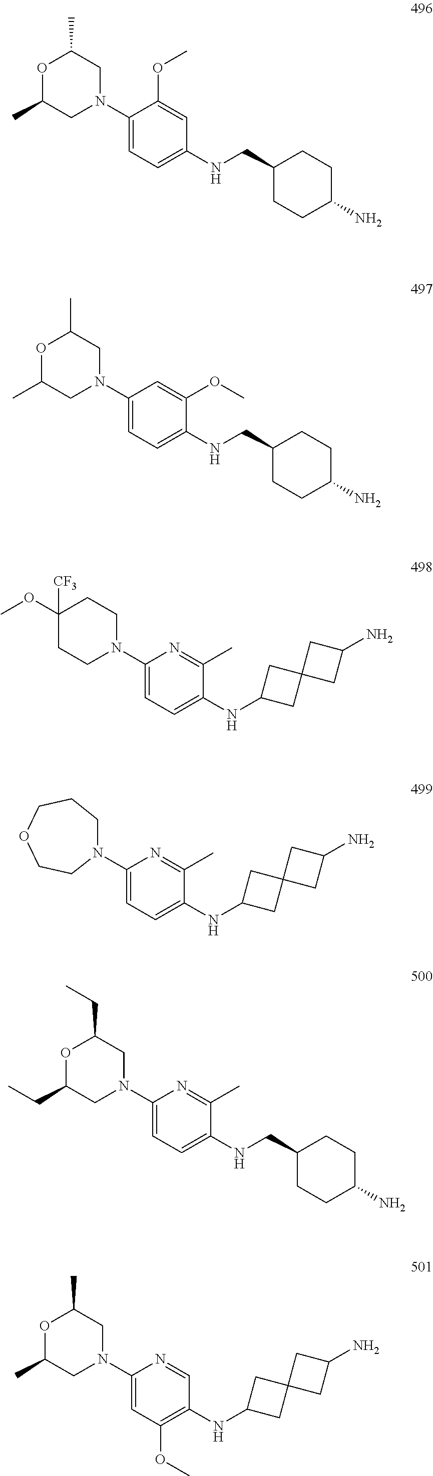 [SMILES: COc1cc(NC[C@H]2CC[C@H](N)CC2)ccc1N1C[C@@H](C)O[C@H](C)C1 ; COc1cc(N2CC(C)OC(C)C2)ccc1NC[C@H]1CC[C@H](N)CC1 ; COC1(C(F)(F)F)CCN(c2ccc(NC3CC4(CC(N)C4)C3)c(C)n2)CC1 ; Cc1nc(N2CCCOCC2)ccc1NC1CC2(CC(N)C2)C1 ; CC[C@@H]1CN(c2ccc(NC[C@H]3CC[C@H](N)CC3)c(C)n2)C[C@H](CC)O1 ; COc1cc(N2C[C@@H](C)O[C@@H](C)C2)ncc1NC1CC2(CC(N)C2)C1] 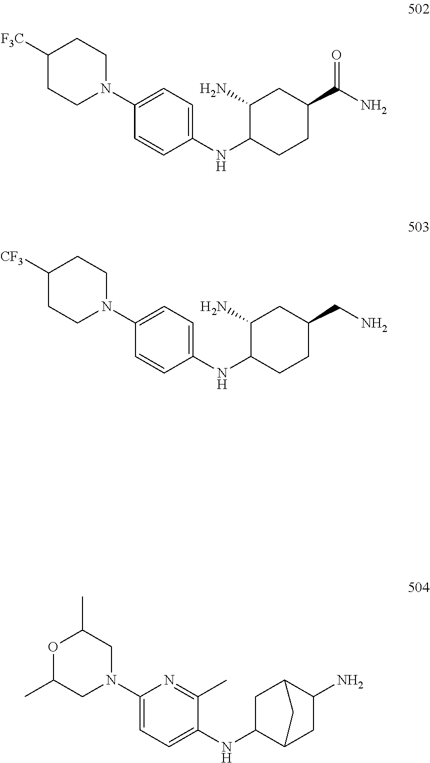 [SMILES: NC(=O)[C@H]1CCC(Nc2ccc(N3CCC(C(F)(F)F)CC3)cc2)[C@H](N)C1 ; NC[C@H]1CCC(Nc2ccc(N3CCC(C(F)(F)F)CC3)cc2)[C@H](N)C1 ; Cc1nc(N2CC(C)OC(C)C2)ccc1NC1CC2CC1CC2N] 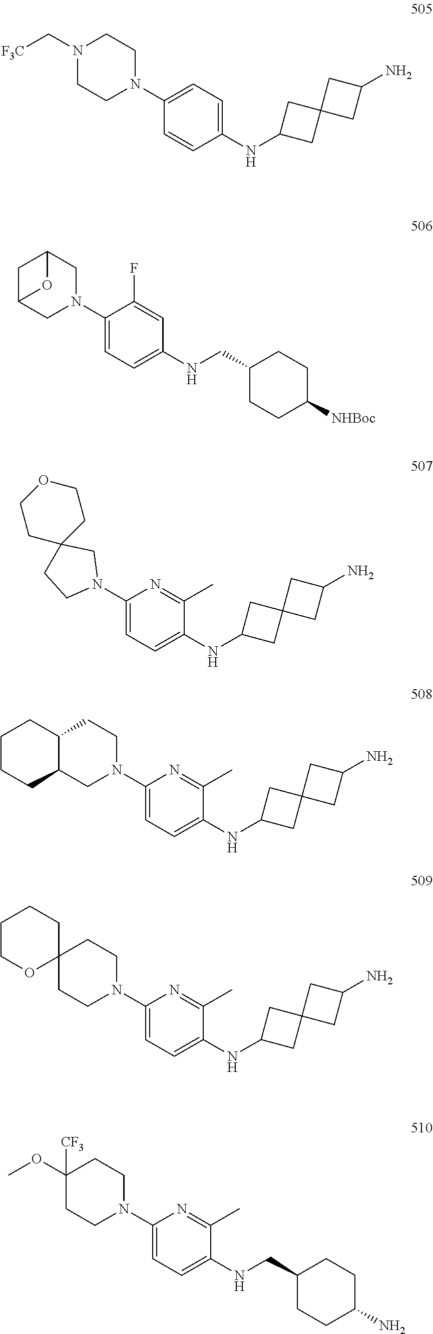 [SMILES: NC1CC2(C1)CC(Nc1ccc(N3CCN(CC(F)(F)F)CC3)cc1)C2 ; CC(C)(C)OC(=O)N[C@H]1CC[C@H](CNc2ccc(N3CC4CC(C3)O4)c(F)c2)CC1 ; Cc1nc(N2CCC3(CCOCC3)C2)ccc1NC1CC2(CC(N)C2)C1 ; Cc1nc(N2CC[C@H]3CCCC[C@@H]3C2)ccc1NC1CC2(CC(N)C2)C1 ; Cc1nc(N2CCC3(CCCCO3)CC2)ccc1NC1CC2(CC(N)C2)C1 ; COC1(C(F)(F)F)CCN(c2ccc(NC[C@H]3CC[C@H](N)CC3)c(C)n2)CC1] 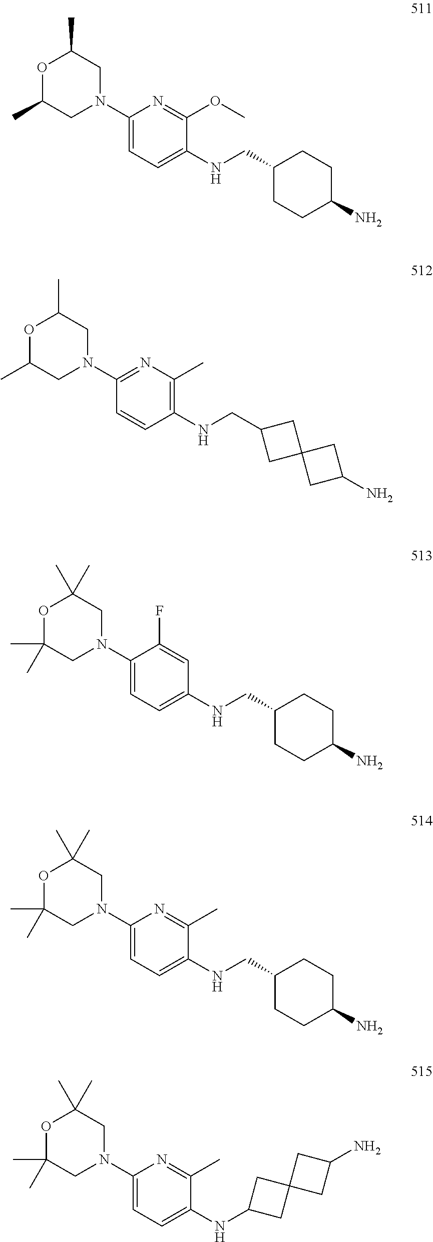 [SMILES: COc1nc(N2C[C@@H](C)O[C@@H](C)C2)ccc1NC[C@H]1CC[C@H](N)CC1 ; Cc1nc(N2CC(C)OC(C)C2)ccc1NCC1CC2(CC(N)C2)C1 ; CC1(C)CN(c2ccc(NC[C@H]3CC[C@H](N)CC3)cc2F)CC(C)(C)O1 ; Cc1nc(N2CC(C)(C)OC(C)(C)C2)ccc1NC[C@H]1CC[C@H](N)CC1 ; Cc1nc(N2CC(C)(C)OC(C)(C)C2)ccc1NC1CC2(CC(N)C2)C1] 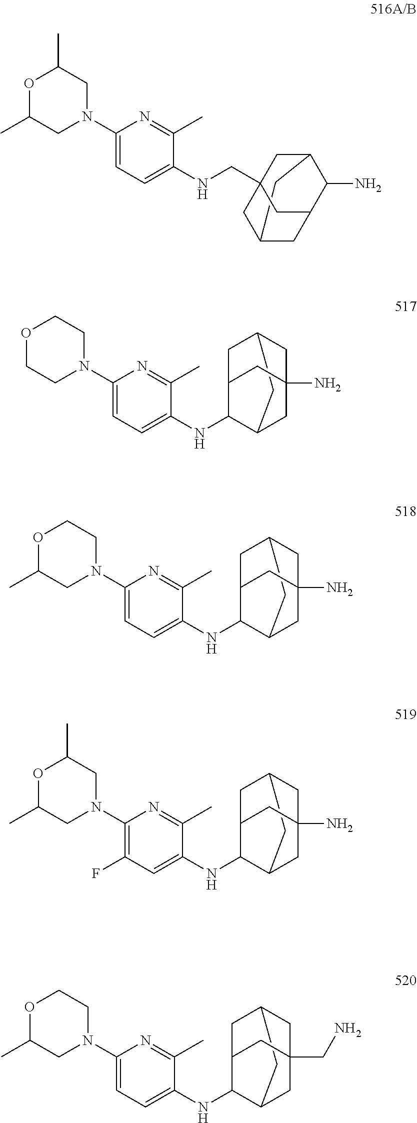 [SMILES: Cc1nc(N2CC(C)OC(C)C2)ccc1NCC12CC3CC(C1)C(N)C(C3)C2 ; Cc1nc(N2CCOCC2)ccc1NC1C2CC3CC1CC(N)(C3)C2 ; Cc1nc(N2CCOC(C)C2)ccc1NC1C2CC3CC1CC(N)(C3)C2 ; Cc1nc(N2CC(C)OC(C)C2)c(F)cc1NC1C2CC3CC1CC(N)(C3)C2 ; Cc1nc(N2CCOC(C)C2)ccc1NC1C2CC3CC1CC(CN)(C3)C2] 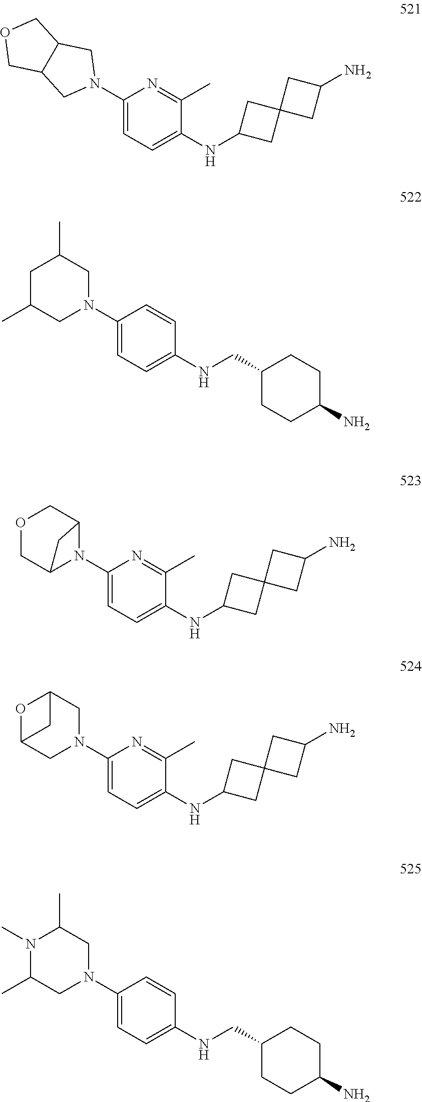 [SMILES: Cc1nc(N2CC3COCC3C2)ccc1NC1CC2(CC(N)C2)C1 ; CC1CC(C)CN(c2ccc(NC[C@H]3CC[C@H](N)CC3)cc2)C1 ; Cc1nc(N2C3COCC2C3)ccc1NC1CC2(CC(N)C2)C1 ; Cc1nc(N2CC3CC(C2)O3)ccc1NC1CC2(CC(N)C2)C1 ; CC1CN(c2ccc(NC[C@H]3CC[C@H](N)CC3)cc2)CC(C)N1C] 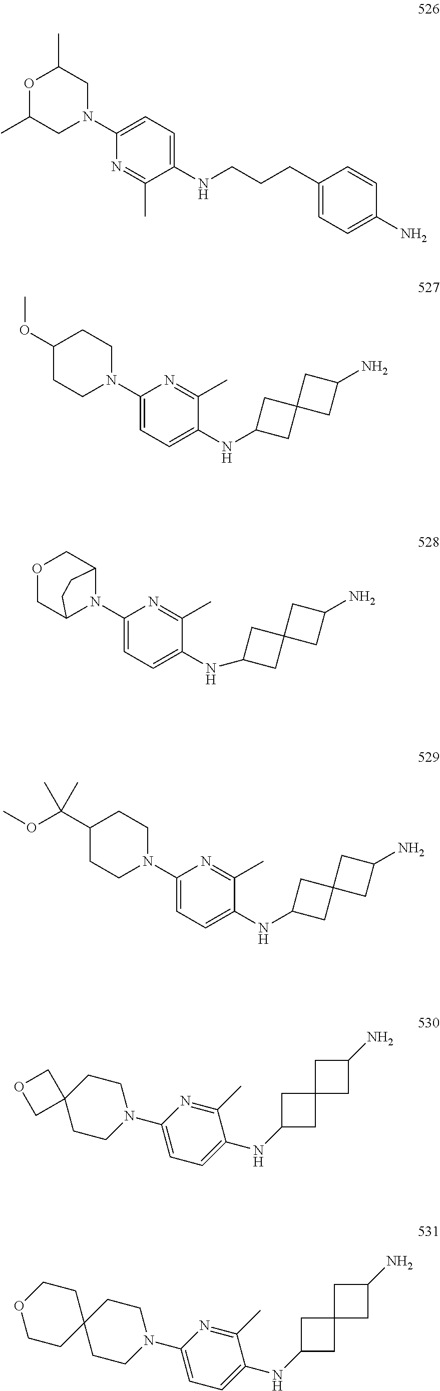 [SMILES: Cc1nc(N2CC(C)OC(C)C2)ccc1NCCCc1ccc(N)cc1 ; COC1CCN(c2ccc(NC3CC4(CC(N)C4)C3)c(C)n2)CC1 ; Cc1nc(N2C3CCC2COC3)ccc1NC1CC2(CC(N)C2)C1 ; COC(C)(C)C1CCN(c2ccc(NC3CC4(CC(N)C4)C3)c(C)n2)CC1 ; Cc1nc(N2CCC3(CC2)COC3)ccc1NC1CC2(CC(N)C2)C1 ; Cc1nc(N2CCC3(CCOCC3)CC2)ccc1NC1CC2(CC(N)C2)C1] 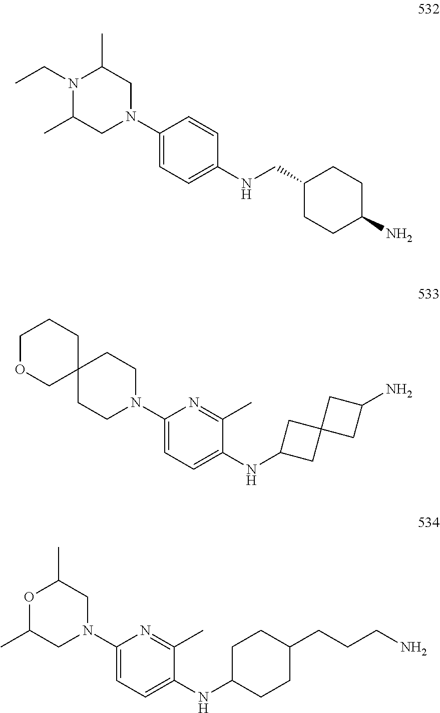 [SMILES: CCN1C(C)CN(c2ccc(NC[C@H]3CC[C@H](N)CC3)cc2)CC1C ; Cc1nc(N2CCC3(CCCOC3)CC2)ccc1NC1CC2(CC(N)C2)C1 ; Cc1nc(N2CC(C)OC(C)C2)ccc1NC1CCC(CCCN)CC1] 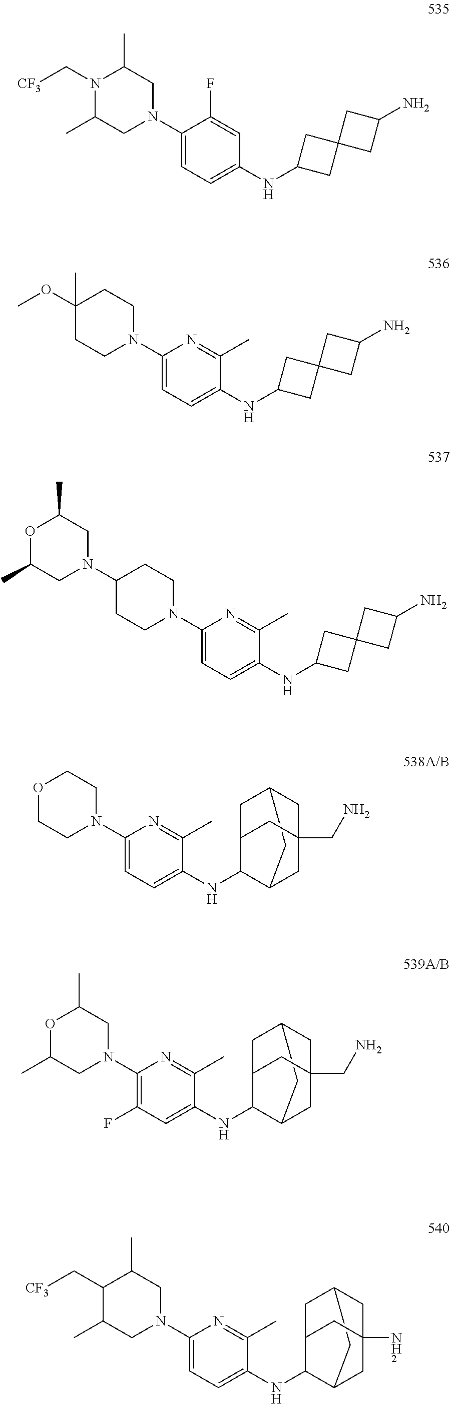 [SMILES: CC1CN(c2ccc(NC3CC4(CC(N)C4)C3)cc2F)CC(C)N1CC(F)(F)F ; COC1(C)CCN(c2ccc(NC3CC4(CC(N)C4)C3)c(C)n2)CC1 ; Cc1nc(N2CCC(N3C[C@@H](C)O[C@@H](C)C3)CC2)ccc1NC1CC2(CC(N)C2)C1 ; Cc1nc(N2CCOCC2)ccc1NC1C2CC3CC1CC(CN)(C3)C2 ; Cc1nc(N2CC(C)OC(C)C2)c(F)cc1NC1C2CC3CC1CC(CN)(C3)C2 ; Cc1nc(N2CC(C)C(CC(F)(F)F)C(C)C2)ccc1NC1C2CC3CC1CC(N)(C3)C2] 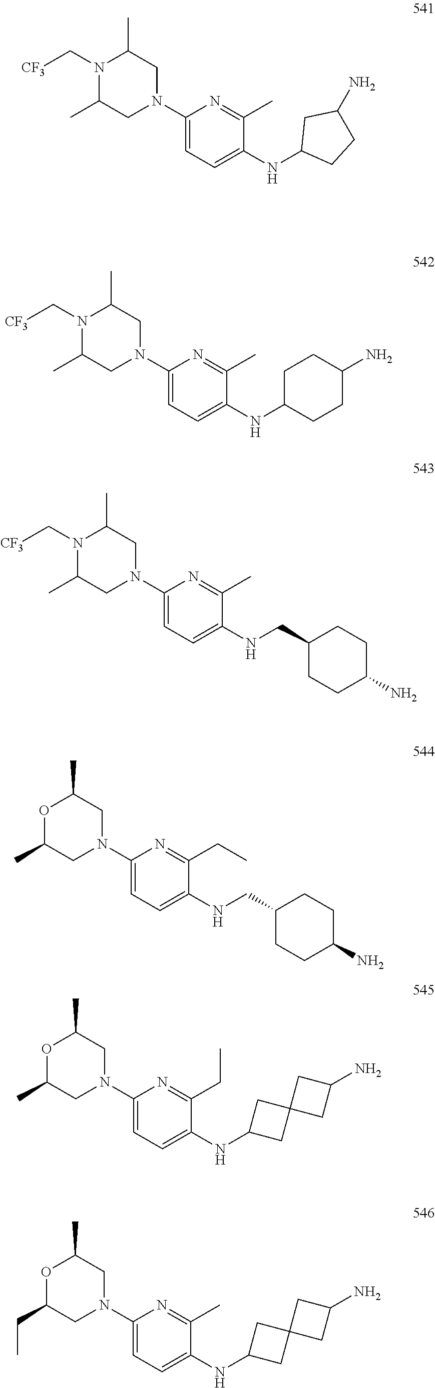 [SMILES: Cc1nc(N2CC(C)N(CC(F)(F)F)C(C)C2)ccc1NC1CCC(N)C1 ; Cc1nc(N2CC(C)N(CC(F)(F)F)C(C)C2)ccc1NC1CCC(N)CC1 ; Cc1nc(N2CC(C)N(CC(F)(F)F)C(C)C2)ccc1NC[C@H]1CC[C@H](N)CC1 ; CCc1nc(N2C[C@@H](C)O[C@@H](C)C2)ccc1NC[C@H]1CC[C@H](N)CC1 ; CCc1nc(N2C[C@@H](C)O[C@@H](C)C2)ccc1NC1CC2(CC(N)C2)C1 ; CC[C@@H]1CN(c2ccc(NC3CC4(CC(N)C4)C3)c(C)n2)C[C@H](C)O1] 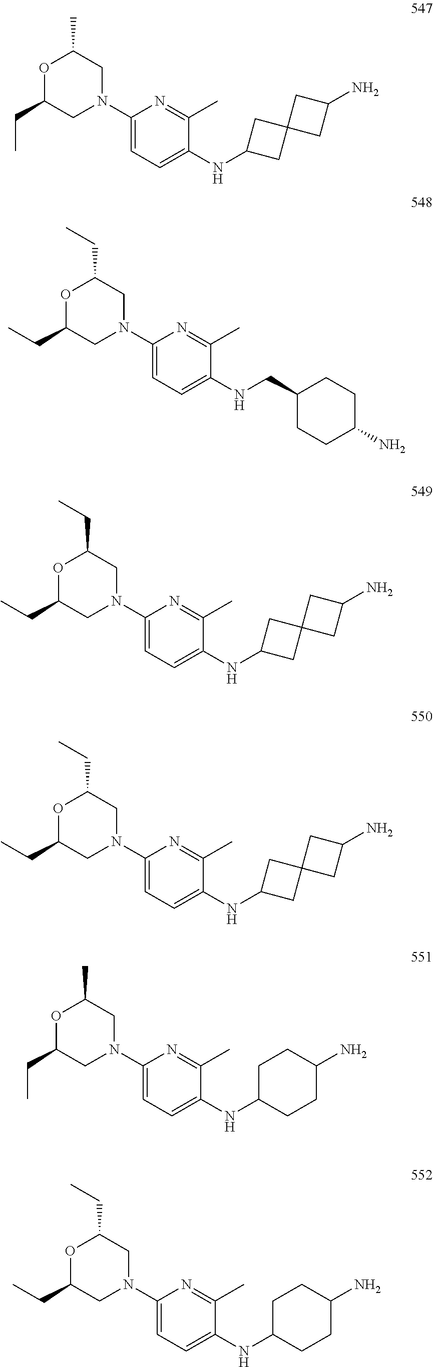 [SMILES: CC[C@@H]1CN(c2ccc(NC3CC4(CC(N)C4)C3)c(C)n2)C[C@@H](C)O1 ; CC[C@@H]1CN(c2ccc(NC[C@H]3CC[C@H](N)CC3)c(C)n2)C[C@@H](CC)O1 ; CC[C@@H]1CN(c2ccc(NC3CC4(CC(N)C4)C3)c(C)n2)C[C@H](CC)O1 ; CC[C@@H]1CN(c2ccc(NC3CC4(CC(N)C4)C3)c(C)n2)C[C@@H](CC)O1 ; CC[C@@H]1CN(c2ccc(NC3CCC(N)CC3)c(C)n2)C[C@H](C)O1 ; CC[C@@H]1CN(c2ccc(NC3CCC(N)CC3)c(C)n2)C[C@@H](CC)O1] 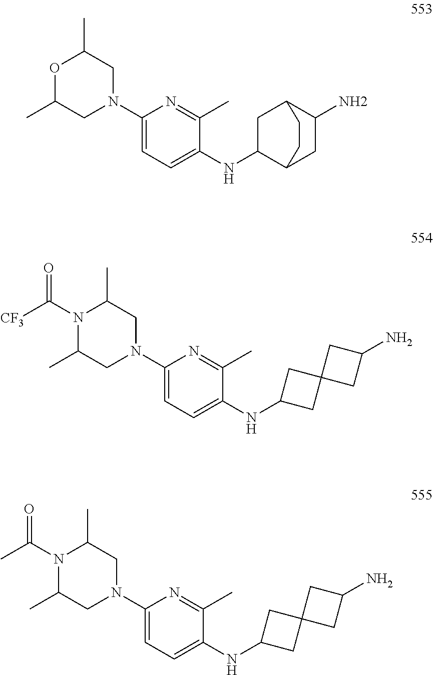 [SMILES: Cc1nc(N2CC(C)OC(C)C2)ccc1NC1CC2CCC1CC2N ; Cc1nc(N2CC(C)N(C(=O)C(F)(F)F)C(C)C2)ccc1NC1CC2(CC(N)C2)C1 ; CC(=O)N1C(C)CN(c2ccc(NC3CC4(CC(N)C4)C3)c(C)n2)CC1C] 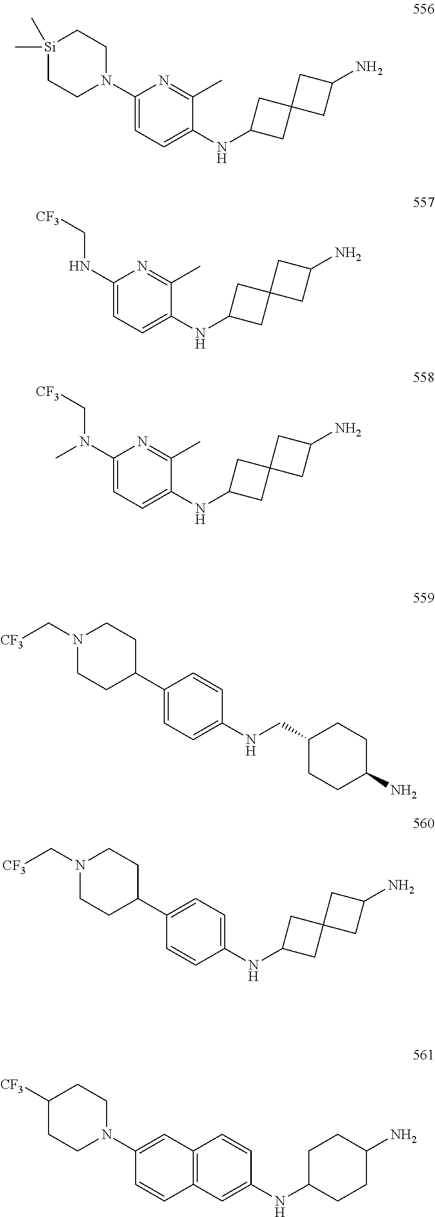 [SMILES: Cc1nc(N2CC[Si](C)(C)CC2)ccc1NC1CC2(CC(N)C2)C1 ; Cc1nc(NCC(F)(F)F)ccc1NC1CC2(CC(N)C2)C1 ; Cc1nc(N(C)CC(F)(F)F)ccc1NC1CC2(CC(N)C2)C1 ; N[C@H]1CC[C@H](CNc2ccc(C3CCN(CC(F)(F)F)CC3)cc2)CC1 ; NC1CC2(C1)CC(Nc1ccc(C3CCN(CC(F)(F)F)CC3)cc1)C2 ; NC1CCC(Nc2ccc3cc(N4CCC(C(F)(F)F)CC4)ccc3c2)CC1] 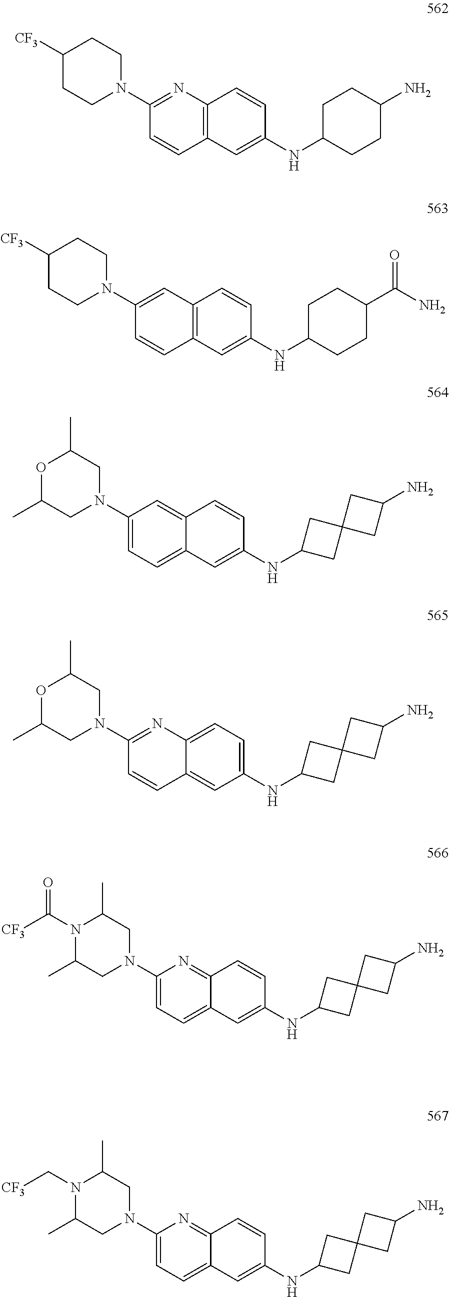 [SMILES: NC1CCC(Nc2ccc3nc(N4CCC(C(F)(F)F)CC4)ccc3c2)CC1 ; NC(=O)C1CCC(Nc2ccc3cc(N4CCC(C(F)(F)F)CC4)ccc3c2)CC1 ; CC1CN(c2ccc3cc(NC4CC5(CC(N)C5)C4)ccc3c2)CC(C)O1 ; CC1CN(c2ccc3cc(NC4CC5(CC(N)C5)C4)ccc3n2)CC(C)O1 ; CC1CN(c2ccc3cc(NC4CC5(CC(N)C5)C4)ccc3n2)CC(C)N1C(=O)C(F)(F)F ; CC1CN(c2ccc3cc(NC4CC5(CC(N)C5)C4)ccc3n2)CC(C)N1CC(F)(F)F] 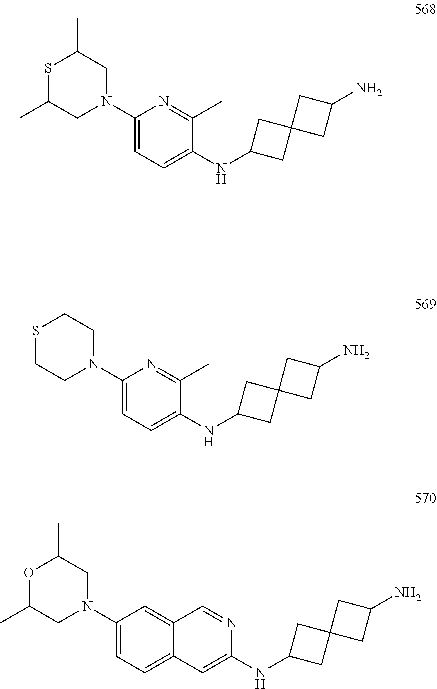 [SMILES: Cc1nc(N2CC(C)SC(C)C2)ccc1NC1CC2(CC(N)C2)C1 ; Cc1nc(N2CCSCC2)ccc1NC1CC2(CC(N)C2)C1 ; CC1CN(c2ccc3cc(NC4CC5(CC(N)C5)C4)ncc3c2)CC(C)O1] 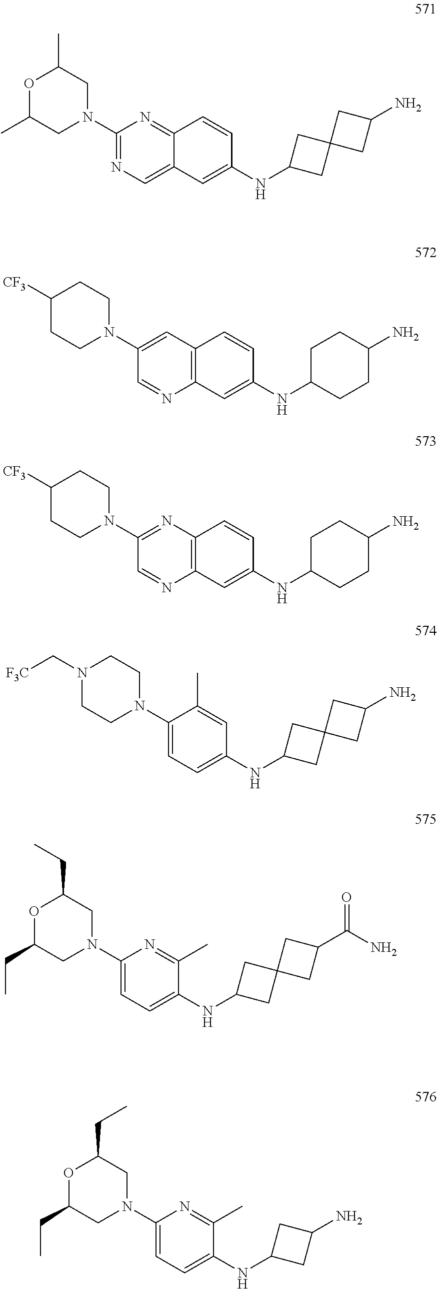 [SMILES: CC1CN(c2ncc3cc(NC4CC5(CC(N)C5)C4)ccc3n2)CC(C)O1 ; NC1CCC(Nc2ccc3cc(N4CCC(C(F)(F)F)CC4)cnc3c2)CC1 ; NC1CCC(Nc2ccc3nc(N4CCC(C(F)(F)F)CC4)cnc3c2)CC1 ; Cc1cc(NC2CC3(CC(N)C3)C2)ccc1N1CCN(CC(F)(F)F)CC1 ; CC[C@@H]1CN(c2ccc(NC3CC4(C3)CC(C(N)=O)C4)c(C)n2)C[C@H](CC)O1 ; CC[C@@H]1CN(c2ccc(NC3CC(N)C3)c(C)n2)C[C@H](CC)O1] 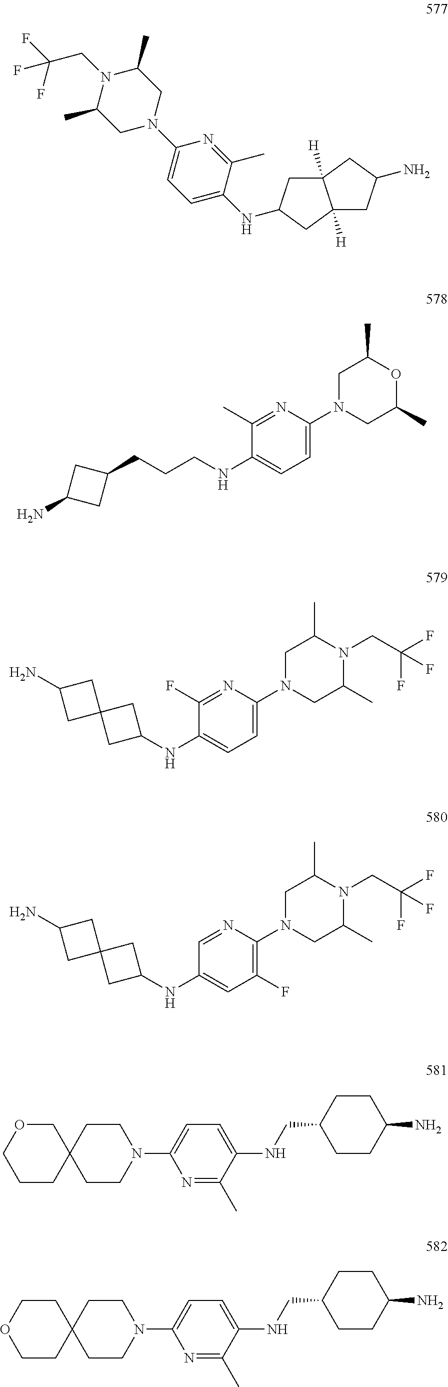 [SMILES: Cc1nc(N2C[C@@H](C)N(CC(F)(F)F)[C@@H](C)C2)ccc1NC1C[C@H]2CC(N)C[C@H]2C1 ; Cc1nc(N2C[C@@H](C)O[C@@H](C)C2)ccc1NCCC[C@H]1C[C@@H](N)C1 ; CC1CN(c2ccc(NC3CC4(CC(N)C4)C3)c(F)n2)CC(C)N1CC(F)(F)F ; CC1CN(c2ncc(NC3CC4(CC(N)C4)C3)cc2F)CC(C)N1CC(F)(F)F ; Cc1nc(N2CCC3(CCCOC3)CC2)ccc1NC[C@H]1CC[C@H](N)CC1 ; Cc1nc(N2CCC3(CCOCC3)CC2)ccc1NC[C@H]1CC[C@H](N)CC1] 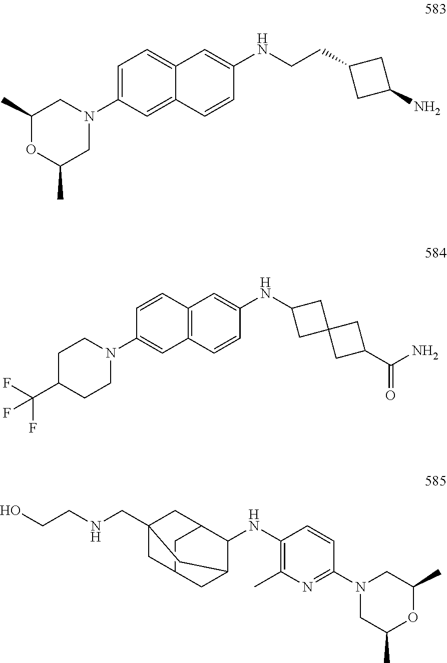 [SMILES: C[C@@H]1CN(c2ccc3cc(NCC[C@H]4C[C@H](N)C4)ccc3c2)C[C@H](C)O1 ; NC(=O)C1CC2(CC(Nc3ccc4cc(N5CCC(C(F)(F)F)CC5)ccc4c3)C2)C1 ; Cc1nc(N2C[C@@H](C)O[C@@H](C)C2)ccc1NC1C2CC3CC1CC(CNCCO)(C3)C2] 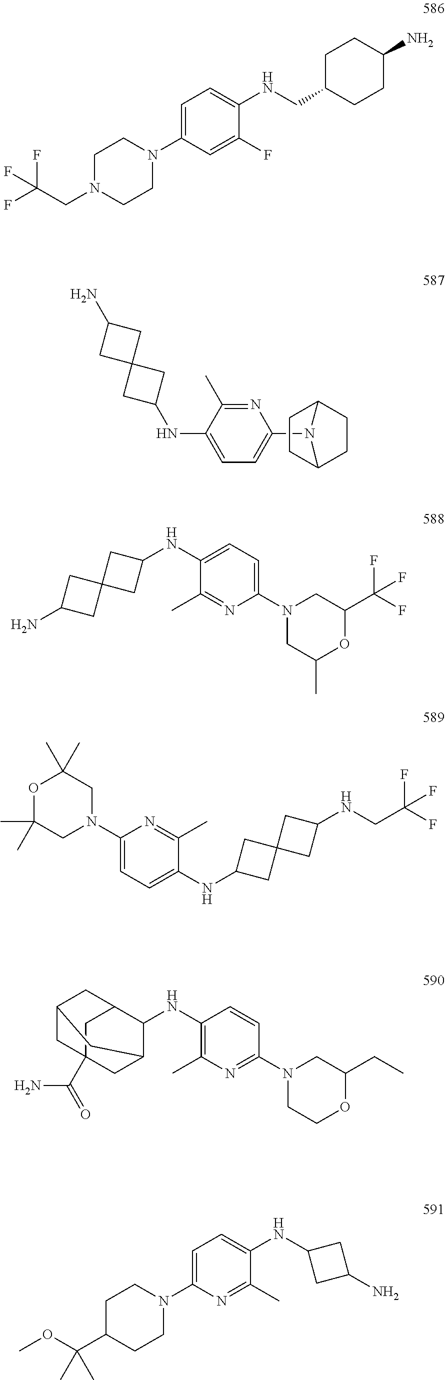 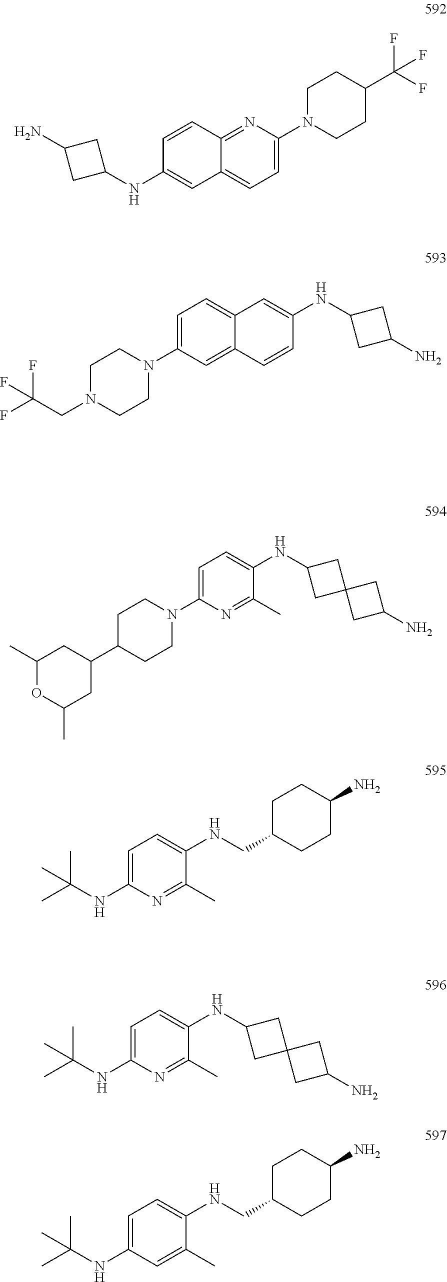 [SMILES: NC1CC(Nc2ccc3nc(N4CCC(C(F)(F)F)CC4)ccc3c2)C1 ; NC1CC(Nc2ccc3cc(N4CCN(CC(F)(F)F)CC4)ccc3c2)C1 ; Cc1nc(N2CCC(C3CC(C)OC(C)C3)CC2)ccc1NC1CC2(CC(N)C2)C1 ; Cc1nc(NC(C)(C)C)ccc1NC[C@H]1CC[C@H](N)CC1 ; Cc1nc(NC(C)(C)C)ccc1NC1CC2(CC(N)C2)C1 ; Cc1cc(NC(C)(C)C)ccc1NC[C@H]1CC[C@H](N)CC1] 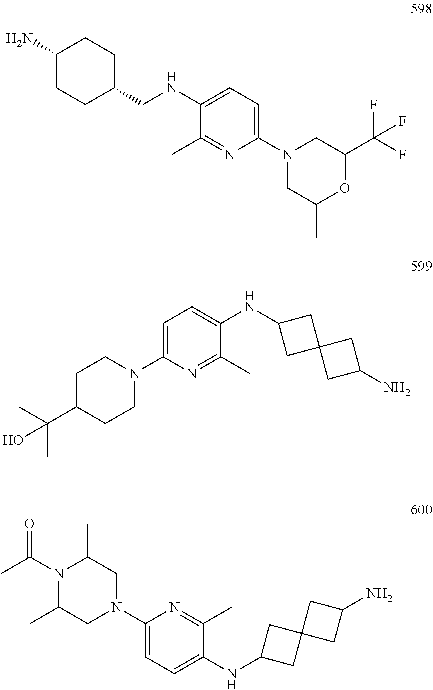 [SMILES: Cc1nc(N2CC(C)OC(C(F)(F)F)C2)ccc1NC[C@H]1CC[C@@H](N)CC1 ; Cc1nc(N2CCC(C(C)(C)O)CC2)ccc1NC1CC2(CC(N)C2)C1 ; CC(=O)N1C(C)CN(c2ccc(NC3CC4(CC(N)C4)C3)c(C)n2)CC1C] 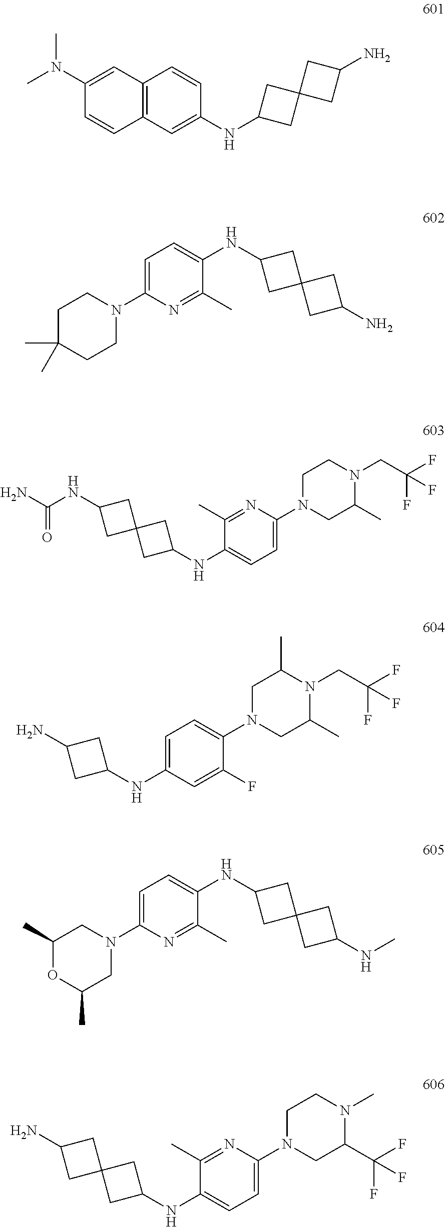 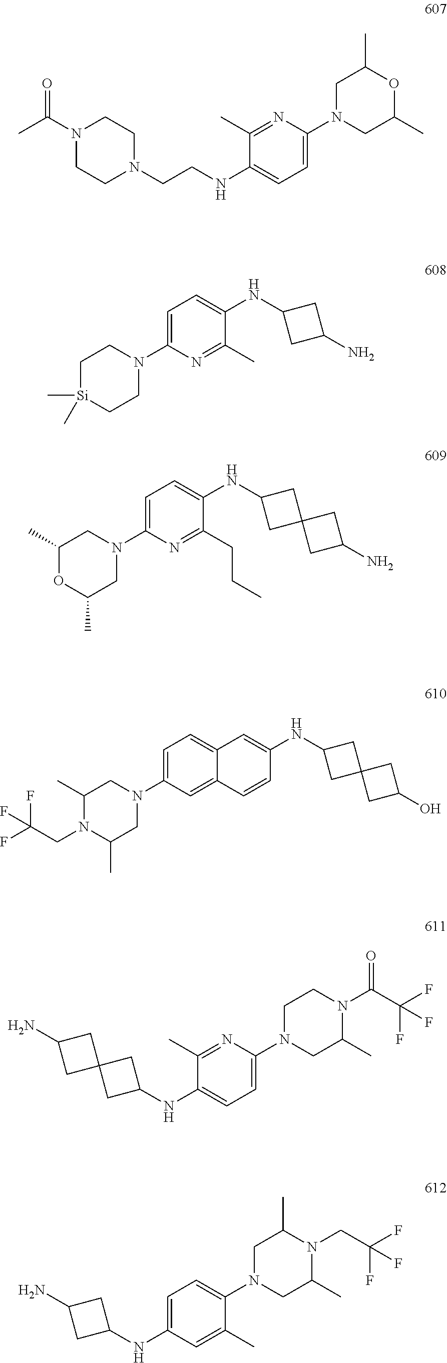 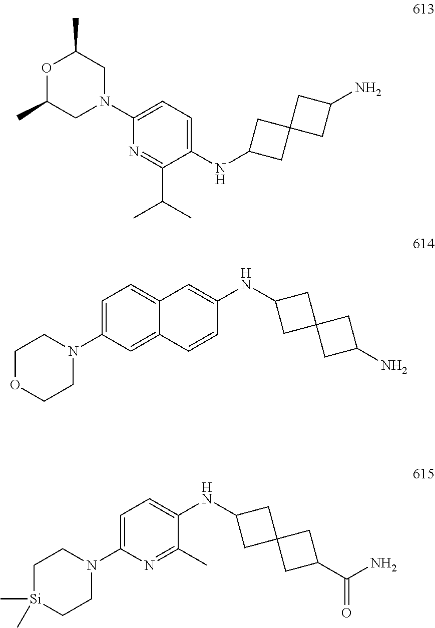 [SMILES: CC(C)c1nc(N2C[C@@H](C)O[C@@H](C)C2)ccc1NC1CC2(CC(N)C2)C1 ; NC1CC2(C1)CC(Nc1ccc3cc(N4CCOCC4)ccc3c1)C2 ; Cc1nc(N2CC[Si](C)(C)CC2)ccc1NC1CC2(C1)CC(C(N)=O)C2] 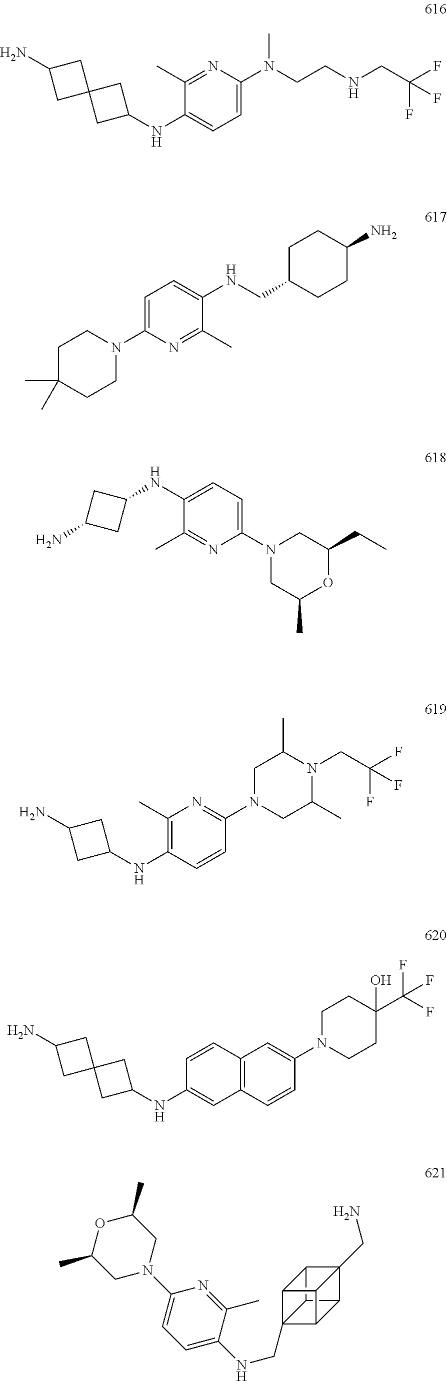 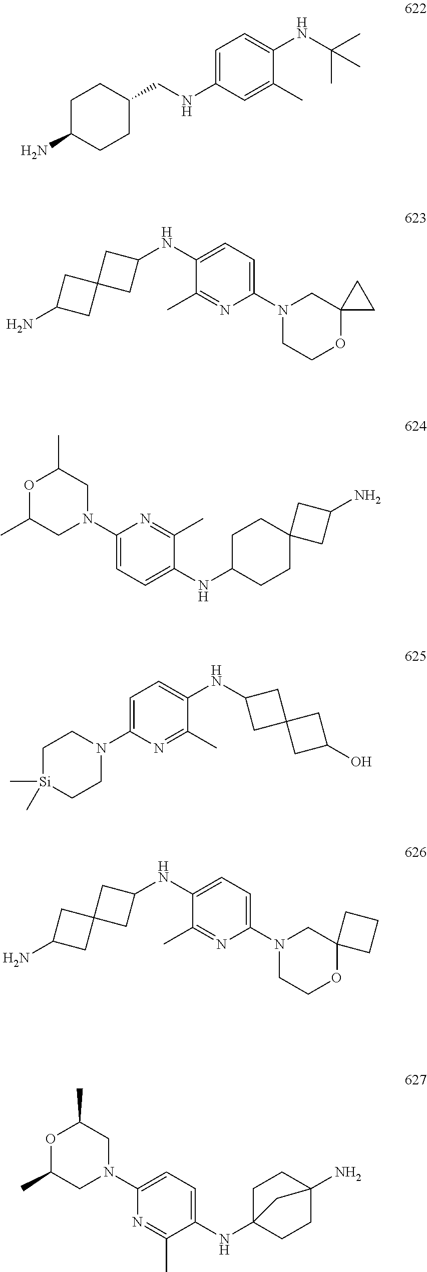 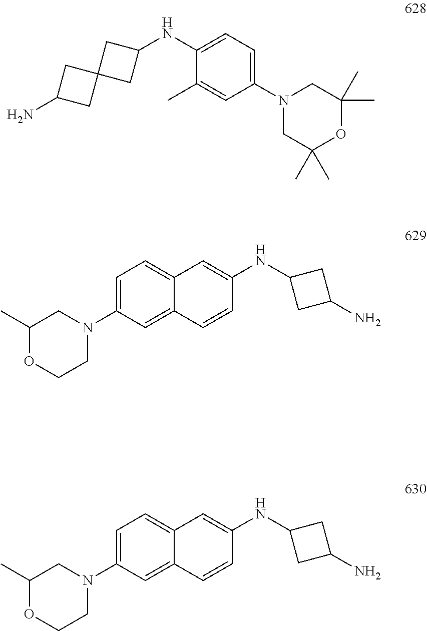 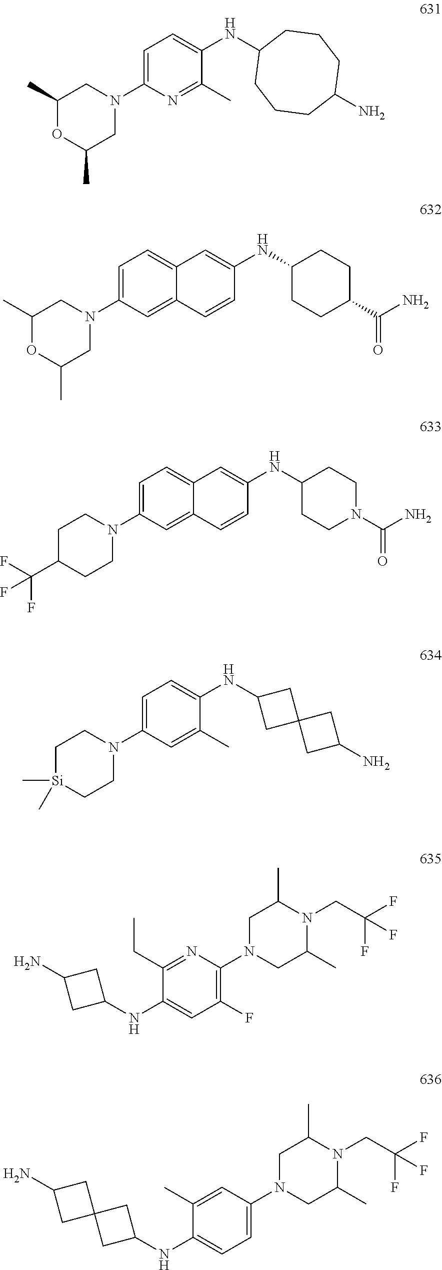 [SMILES: Cc1nc(N2C[C@@H](C)O[C@@H](C)C2)ccc1NC1CCCC(N)CCC1 ; CC1CN(c2ccc3cc(N[C@H]4CC[C@@H](C(N)=O)CC4)ccc3c2)CC(C)O1 ; NC(=O)N1CCC(Nc2ccc3cc(N4CCC(C(F)(F)F)CC4)ccc3c2)CC1 ; Cc1cc(N2CC[Si](C)(C)CC2)ccc1NC1CC2(CC(N)C2)C1 ; CCc1nc(N2CC(C)N(CC(F)(F)F)C(C)C2)c(F)cc1NC1CC(N)C1 ; Cc1cc(N2CC(C)N(CC(F)(F)F)C(C)C2)ccc1NC1CC2(CC(N)C2)C1] 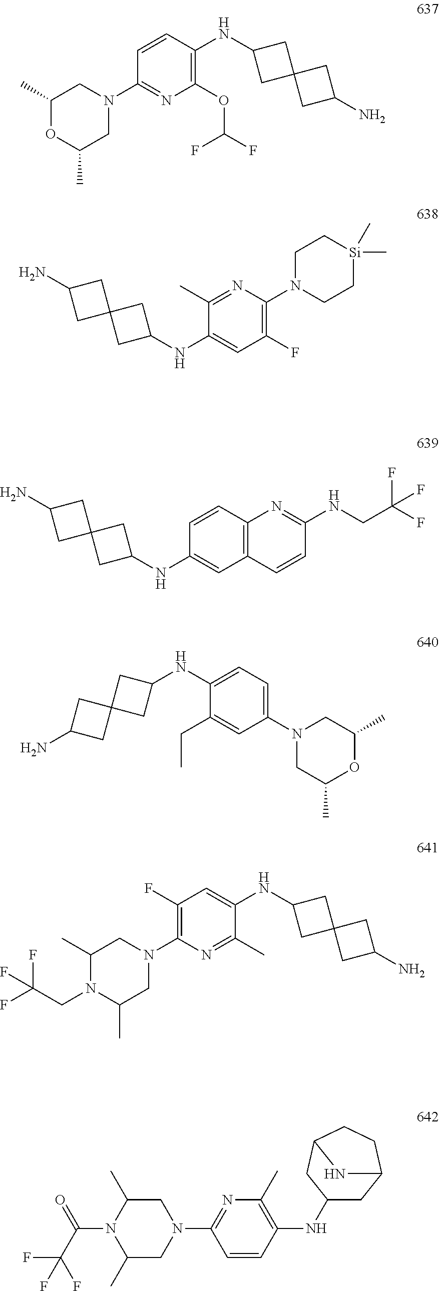 [SMILES: C[C@@H]1CN(c2ccc(NC3CC4(CC(N)C4)C3)c(OC(F)F)n2)C[C@H](C)O1 ; Cc1nc(N2CC[Si](C)(C)CC2)c(F)cc1NC1CC2(CC(N)C2)C1 ; NC1CC2(C1)CC(Nc1ccc3nc(NCC(F)(F)F)ccc3c1)C2 ; CCc1cc(N2C[C@@H](C)O[C@@H](C)C2)ccc1NC1CC2(CC(N)C2)C1 ; Cc1nc(N2CC(C)N(CC(F)(F)F)C(C)C2)c(F)cc1NC1CC2(CC(N)C2)C1 ; Cc1nc(N2CC(C)N(C(=O)C(F)(F)F)C(C)C2)ccc1NC1CC2CCC(C1)N2] 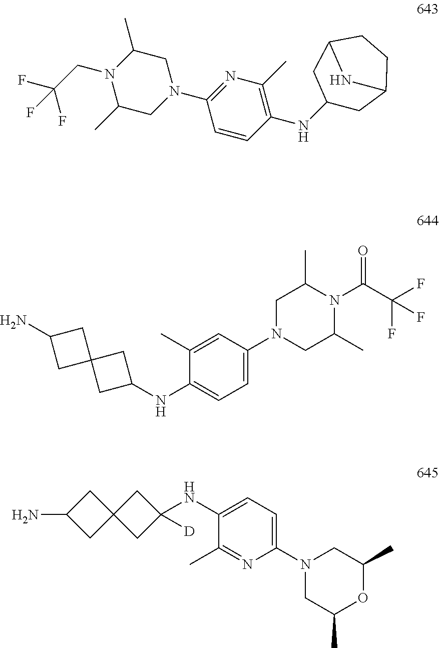 [SMILES: Cc1nc(N2CC(C)N(CC(F)(F)F)C(C)C2)ccc1NC1CC2CCC(C1)N2 ; Cc1cc(N2CC(C)N(C(=O)C(F)(F)F)C(C)C2)ccc1NC1CC2(CC(N)C2)C1 ; [2H]C1(Nc2ccc(N3C[C@@H](C)O[C@@H](C)C3)nc2C)CC2(CC(N)C2)C1] 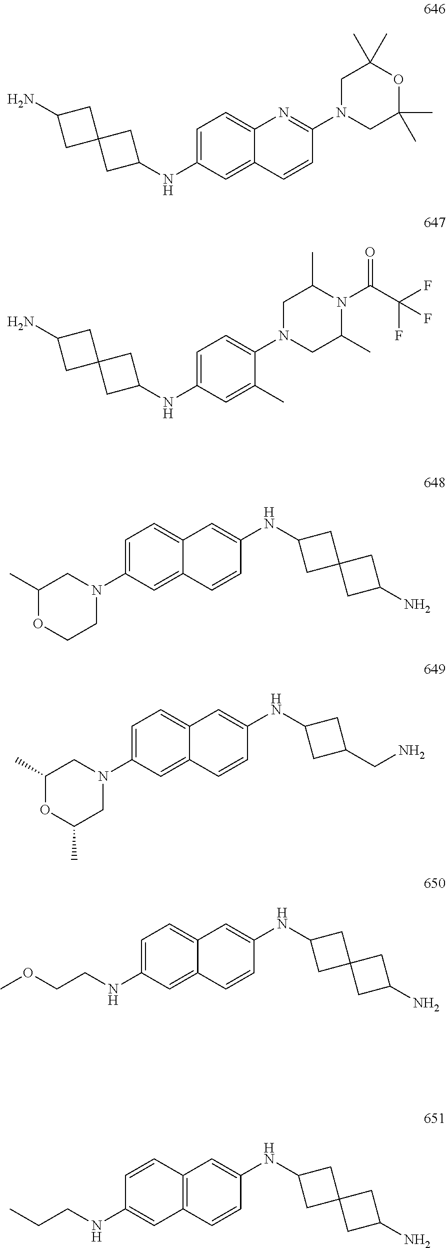 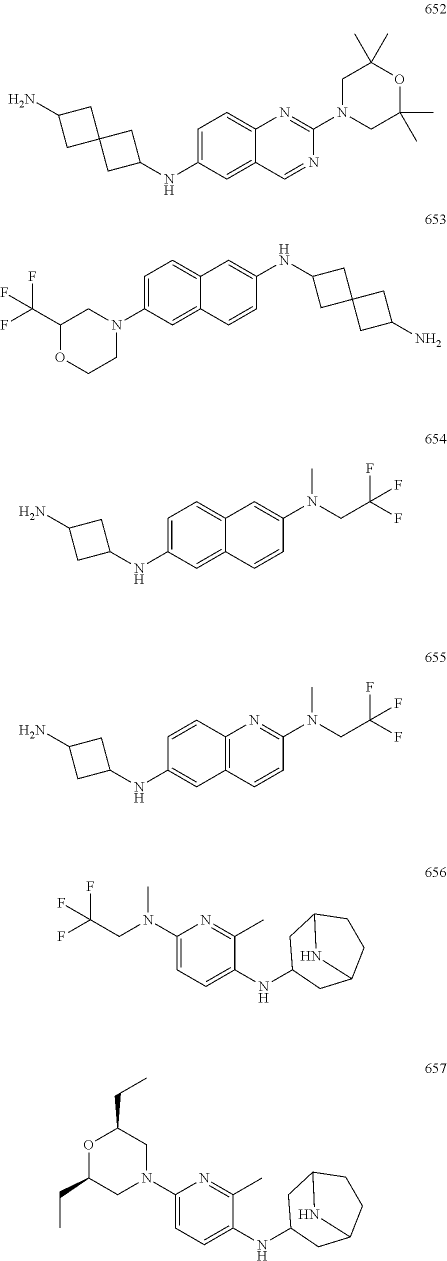 [SMILES: CC1(C)CN(c2ncc3cc(NC4CC5(CC(N)C5)C4)ccc3n2)CC(C)(C)O1 ; NC1CC2(C1)CC(Nc1ccc3cc(N4CCOC(C(F)(F)F)C4)ccc3c1)C2 ; CN(CC(F)(F)F)c1ccc2cc(NC3CC(N)C3)ccc2c1 ; CN(CC(F)(F)F)c1ccc2cc(NC3CC(N)C3)ccc2n1 ; Cc1nc(N(C)CC(F)(F)F)ccc1NC1CC2CCC(C1)N2 ; CC[C@@H]1CN(c2ccc(NC3CC4CCC(C3)N4)c(C)n2)C[C@H](CC)O1] 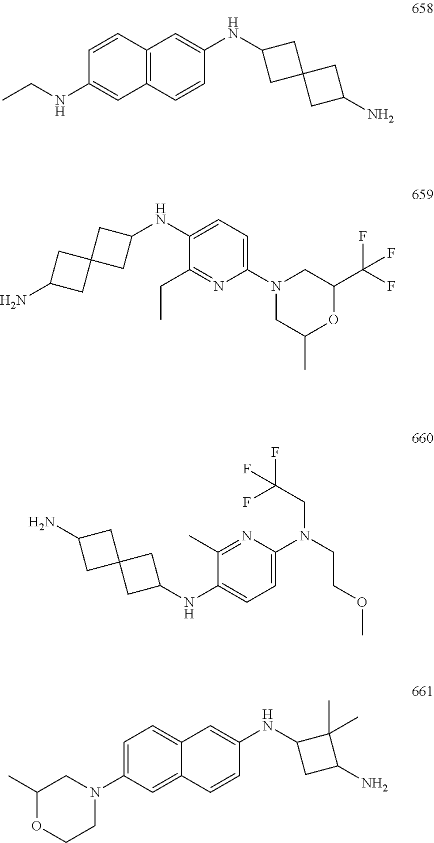
a tautomer thereof, a solvate or stereoisomer of the compound or the tautomer, or a pharmaceutically acceptable salt of the foregoing.

9. A pharmaceutical composition comprising a compound according to claim 1, a tautomer thereof, a solvate or stereoisomer of the compound or the tautomer, or a pharmaceutically acceptable salt of the foregoing and at least one pharmaceutically acceptable carrier.

10. A method of treating a disease or condition involving ferroptosis, or modulating ferroptosis,
comprising administering to a subject in need thereof, a therapeutically effective amount of a compound according to claim 1, a tautomer thereof, a solvate or stereoisomer of the compound or the tautomer, or a pharmaceutically acceptable salt of the foregoing or a pharmaceutical composition thereof; or
contacting a subject in need thereof with a compound according to claim 1, a tautomer thereof, a solvate or stereoisomer of the compound or the tautomer, or a pharmaceutically acceptable salt of the foregoing or a pharmaceutical composition thereof.

11. The compound of claim 6, wherein n is 0, q is 0, and p is 0, 1, 2, or 3.

12. The method of claim 10, wherein the disease or condition involving ferroptosis is selected from neuropathy, stroke, neurodegenerative disease, Parkinson's Disease, amyotrophic lateral sclerosis (AML), multiple sclerosis, Huntington's Disease, dementia with Lewy bodies, Friedreich's ataxia, hair follicle morphogenesis, diabetes, sepsis, transplant rejection, periventricular leukomalacia, ischemia reperfusion injury, blood coagulation, myocardial infarction, and kidney dysfunction.
